SEQUENCE LISTING (49 sequences)

SEQ ID NO: 1 - protein, 120 aa, synthetic construct [sequence omitted]

SEQ ID NO: 2 - protein, 107 aa, synthetic construct [sequence omitted]

SEQ ID NO: 3 - protein, 119 aa, synthetic construct [sequence omitted]

SEQ ID NO: 4 - protein, 107 aa, synthetic construct [sequence omitted]

SEQ ID NO: 5 - protein, 7 aa, synthetic construct
  source 1..7: note = Trastuzumab Chothia CDR H1
GFNIKDT

SEQ ID NO: 6 - protein, 6 aa, synthetic construct
  source 1..6: note = Trastuzumab Chothia CDR H2
YPTNGY

SEQ ID NO: 7 - protein, 11 aa, synthetic construct
  source 1..11: note = Trastuzumab Chothia/kabat/AbM CDR H3
WGGDGFYAMDY

SEQ ID NO: 8 - protein, 11 aa, synthetic construct
  source 1..11: note = Trastuzumab Chothia/Kabat/AbM CDR L1
RASQDVNTAVA

SEQ ID NO: 9 - protein, 7 aa, synthetic construct
  source 1..7: note = Trastuzumab Chothia/Kabat/AbM CDR L2
SASFLYS

SEQ ID NO: 10 - protein, 9 aa, synthetic construct
  source 1..9: note = Trastuzumab Chothia/Kabat/IMGT/AbM CDR L3
QQHYTTPPT

SEQ ID NO: 11 - protein, 5 aa, synthetic construct
  source 1..5: note = Trastuzumab Kabat CDR H1
DTYIH

SEQ ID NO: 12 - protein, 17 aa, synthetic construct
  source 1..17: note = Trastuzumab Kabat CDR H2
RIYPTNGYTRYADSVKG

SEQ ID NO: 13 - protein, 8 aa, synthetic construct
  source 1..8: note = Trastuzumab IMGT CDR H1
GFNIKDTY

SEQ ID NO: 14 - protein, 8 aa, synthetic construct
  source 1..8: note = Trastuzumab IMGT CDR H2
IYPTNGYT

SEQ ID NO: 15 - protein, 13 aa, synthetic construct
  source 1..13: note = Trastuzumab IMGT CDR H3
SRWGGDGFYAMDY

SEQ ID NO: 16 - protein, 6 aa, synthetic construct
  source 1..6: note = Trastuzumab IMGT CDR L1
QDVNTA

SEQ ID NO: 17 - skipped

SEQ ID NO: 18 - protein, 10 aa, synthetic construct
  source 1..10: note = Trastuzumab AbM CDR H1
GFNIKDTYIH

SEQ ID NO: 19 - protein, 10 aa, synthetic construct
  source 1..10: note = Trastuzumab AbM CDR H2
RIYPTNGYTR

SEQ ID NO: 20 - protein, 7 aa, synthetic construct
  source 1..7: note = Pertuzumab Chothia CDR H1
GFTFTDY

SEQ ID NO: 21 - protein, 6 aa, synthetic construct
  source 1..6: note = Pertuzumab Chothia CDR H2
NPNSGG

SEQ ID NO: 22 - protein, 10 aa, synthetic construct
  source 1..10: note = Pertuzumab Chothia/Kabat/AbM CDR H3
NLGPSFYFDY

SEQ ID NO: 23 - protein, 11 aa, synthetic construct
  source 1..11: note = Pertuzumab Chothia/Kabat/AbM CDR L1
KASQDVSIGVA

SEQ ID NO: 24 - protein, 7 aa, synthetic construct
  source 1..7: note = Pertuzumab Chothia/Kabat/AbM CDR L2
SASYRYT

SEQ ID NO: 25 - protein, 9 aa, synthetic construct
  source 1..9: note = Pertuzumab Chothia/Kabat/IMGT/AbM CDR L3
QQYYIYPYT

SEQ ID NO: 26 - protein, 5 aa, synthetic construct
  source 1..5: note = Pertuzumab Kabat CDR H1
DYTMD

SEQ ID NO: 27 - protein, 17 aa, synthetic construct
  source 1..17: note = Pertuzumab Kabat CDR H2
DVNPNSGGSIYNQRFKG

SEQ ID NO: 28 - protein, 8 aa, synthetic construct
  source 1..8: note = Pertuzumab IMGT CDR H1
GFTFTDYT

SEQ ID NO: 29 - protein, 8 aa, synthetic construct
  source 1..8: note = Pertuzumab IMGT CDR H2
VNPNSGGS

SEQ ID NO: 30 - protein, 12 aa, synthetic construct
  source 1..12: note = Pertuzumab IMGT CDR H3
ARNLGPSFYFDY

SEQ ID NO: 31 - protein, 6 aa, synthetic construct
  source 1..6: note = Pertuzumab IMGT CDR L1
QDVSIG

SEQ ID NO: 32 - skipped

SEQ ID NO: 33 - protein, 10 aa, synthetic construct
  source 1..10: note = Pertuzumab AbM CDR H1
GFTFTDYTMD

SEQ ID NO: 34 - protein, 10 aa, synthetic construct
  source 1..10: note = Pertuzumab AbM CDR H2
DVNPNSGGSI

SEQ ID NO: 35 - protein, 330 aa, synthetic construct
  source 1..330: note = CH of Trastuzumab/Pertuzumab
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 107 aa, synthetic construct [sequence omitted]

SEQ ID NO: 37 - protein, 450 aa, synthetic construct
  source 1..450: note = heavy chain of Trastuzumab
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
  source 1..214: note = light chain of Trastuzumab
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 449 aa, synthetic construct
  source 1..449: note = heavy chain of Pertuzumab
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, synthetic construct
  source 1..214: note = light chain of Pertuzumab
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 4 aa, synthetic construct
GGFG

SEQ ID NO: 42 - protein, 4 aa, synthetic construct
GGVA

SEQ ID NO: 43 - protein, 4 aa, synthetic construct
GFLG

SEQ ID NO: 44 - protein, 4 aa, synthetic construct
EAAA

SEQ ID NO: 45 - protein, 5 aa, synthetic construct
GGGGG

SEQ ID NO: 46 - protein, 5 aa, synthetic construct
DGGFG

SEQ ID NO: 47 - protein, 5 aa, synthetic construct
EGGFG

SEQ ID NO: 48 - protein, 5 aa, synthetic construct
GGGGS

SEQ ID NO: 49 - protein, 20 aa, synthetic construct
GGGGSGGGGSGGGGSGGGGS

(19) United States
(12) Patent Application Publication
(10) Pub. No.: US 2026/0263615 A1
(43) Pub. Date: Sep. 10, 2026

(54) COMPOUND HAVING BIOLOGICAL ACTIVITY, AND PREPARATION METHOD THEREFOR AND USE THEREOF

(71) Applicant: Sichuan Kelun-Biotech Biopharmaceutical Co., Ltd., Chengdu, Sichuan (CN)

(72) Inventors: Qiang Tian, Chengdu, Sichuan (CN); Yitao Zhang, Chengdu, Sichuan (CN); Jian Ye, Chengdu, Sichuan (CN); Dong Long, Chengdu, Sichuan (CN); Qin Zhou, Chengdu, Sichuan (CN); Hu Long, Chengdu, Sichuan (CN); Yu Yang, Chengdu, Sichuan (CN); Xiaoxi Yuan, Chengdu, Sichuan (CN); Hongmei Song, Chengdu, Sichuan (CN); Junyou Ge, Chengdu, Sichuan (CN)

(21) Appl. No.: 19/469,628

(22) PCT Filed: May 10, 2024
(86) PCT No.: PCT/CN2024/092433

§ 371 Date: Sep. 26, 2025

(30) Foreign Application Priority Data
May 12, 2023 (CN) 202310538792.7
Sep. 27, 2023 (CN) 202311265967.8
Feb. 6, 2024 (CN) 202410172424.X

(51) Int. Cl. A61K 47/68; A61P 35/00; C07K 16/32

(52) U.S. Cl. CPC A61K 47/68037; A61K 47/6855; A61K 47/6889; A61P 35/00; C07K 16/32

(57) ABSTRACT

A compound having a biological activity, and a preparation method therefor and the use thereof. The compound has a structure as shown in formula M′-L-E-D, the compound can be used for preparing an antibody-drug conjugate. The conjugate has a better drug-to-antibody conjugation ratio, and also has a good targeted killing effect on solid tumors such as gastric cancer, breast cancer, lung cancer and urothelial carcinoma.

The present application is based on and claims priority to the Chinese application with application number CN 202310538792.7 (filed on May 12, 2023), the Chinese application with application number CN 202311265967.8 (filed on Sep. 27, 2023), and the Chinese application with application number CN 202410172424.X (filed on Feb. 6, 2024). All the contents of these Chinese applications are hereby incorporated into the present application in their entirety.

### TECHNICAL FIELD

The present application relates to the pharmaceutical field, and specifically to a compound having biological activity, and preparation method therefor and use thereof.

### BACKGROUND ART

Antibody-drug conjugate (ADC) is typically composed of a monoclonal antibody, a bioactive molecule (primarily a tumor-killing cytotoxin) and a linker. The bioactive molecule is covalently coupled to the antibody via the linker. The antibody recognizes a specific target on the surface of tumor cells, directing the ADC to the tumor microenvironment and cancer cell surfaces. Through endocytosis, the ADC enters the cancer cells, where the bioactive molecule is released. By inhibiting the tubulin of the cancer cells or damaging the DNA of the cancer cells, the bioactive molecule kills the cancer cells while minimizing damage to normal tissue cells.

As an important component of antibody-drug conjugates, the drug-linker has an important impact on the overall efficacy and safety of the ADC drug. For example, both Disitamab vedotin and Trastuzumab deruxtecan utilize the maleimide joint MC in their linker moieties. This joint has been reported to be susceptible to reversible Michael addition and thiol exchange reactions under physiological conditions, resulting in reduced efficacy and increased toxicity (Nat Biotechnol. 2012, 32, 184-189; Bioconjugate Chem. 2015, 26, 145-152). Therefore, there are still needs in the art to develop a new drug linker structure for preparing antibody-drug conjugates with good efficacy and safety.

### CONTENTS OF THE INVENTION

The present application relates to a condensed ring compound, which has a structure represented by the general formula D-E-L-M′. The compound can be used to prepare an antibody-drug conjugate, and the conjugate has a better drug-to-antibody conjugation ratio (for example, 6.5 to 8.5). Therefore, the present application provides a condensed ring compound, which can be used to prepare an antibody-drug conjugate, and the conjugate has a good conjugation ratio, excellent efficacy, safety and pharmacokinetic properties.

### Antibody-Drug Conjugate

In one aspect, the present application provides a compound or pharmaceutically acceptable salt thereof, which has a structure represented by the formula D-E-L-M′, wherein:
M′ represents -M-Lg, wherein Lg is a leaving group for a nucleophilic substitution reaction, and M is a structural fragment capable of binding to a target moiety;
L represents a structural fragment connecting M and E;
E represents a structural fragment connecting L and D;
D represents a cytotoxic drug fragment.

In some embodiments, M is selected from the following substituted or unsubstituted structural fragments:

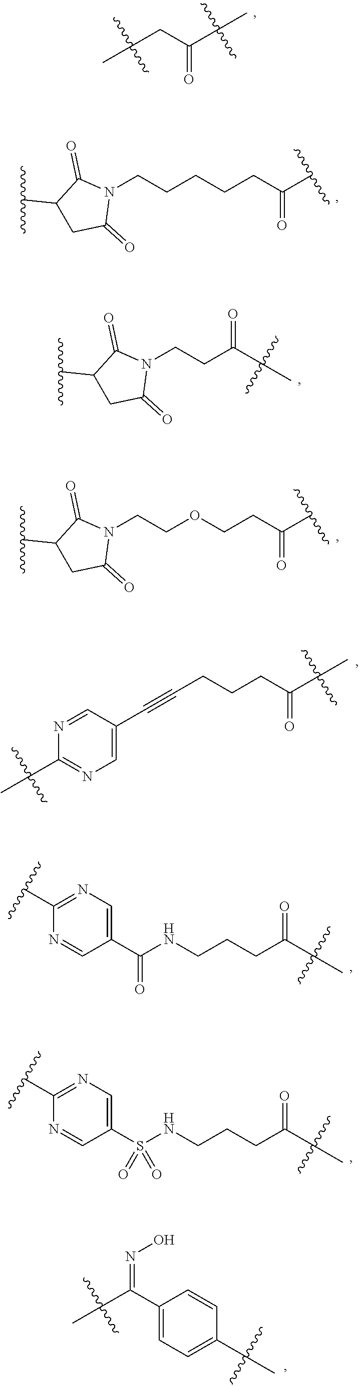

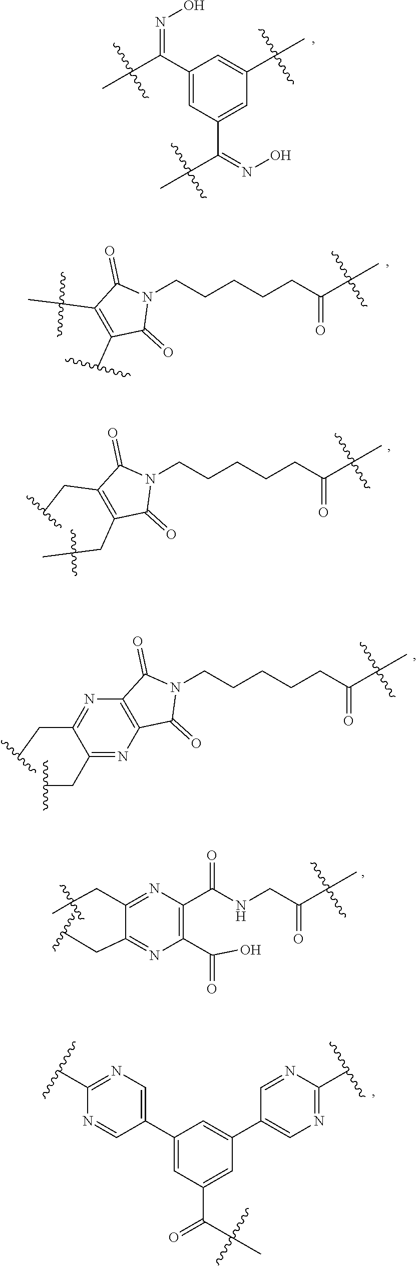 [SMILES: */C(=N/O)c1cc(*)cc(/C(*)=N\O)c1 ; *C(=O)CCCCCN1C(=O)C(*)=C(*)C1=O ; *CC1=C(C*)C(=O)N(CCCCCC(*)=O)C1=O ; *Cc1nc2c(nc1C*)C(=O)N(CCCCCC(*)=O)C2=O ; *Cc1nc(C(=O)O)c(C(=O)NCC(*)=O)nc1C* ; *C(=O)c1cc(-c2cnc(*)nc2)cc(-c2cnc(*)nc2)c1]

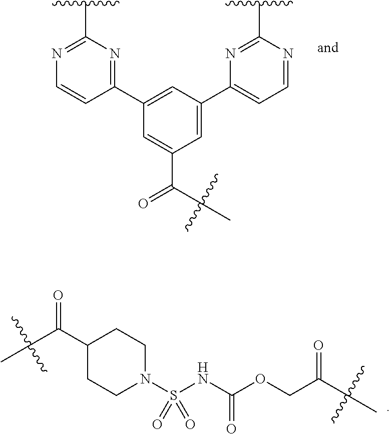 [SMILES: *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the following substituted or unsubstituted structural fragments:

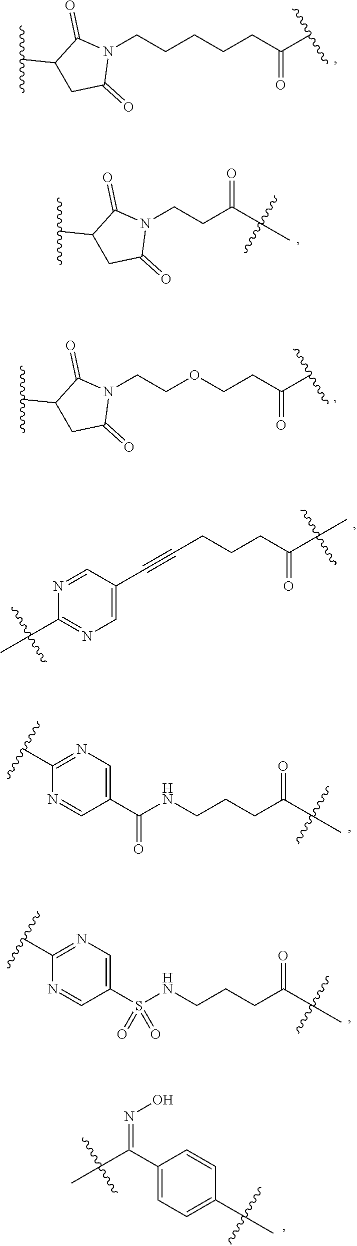

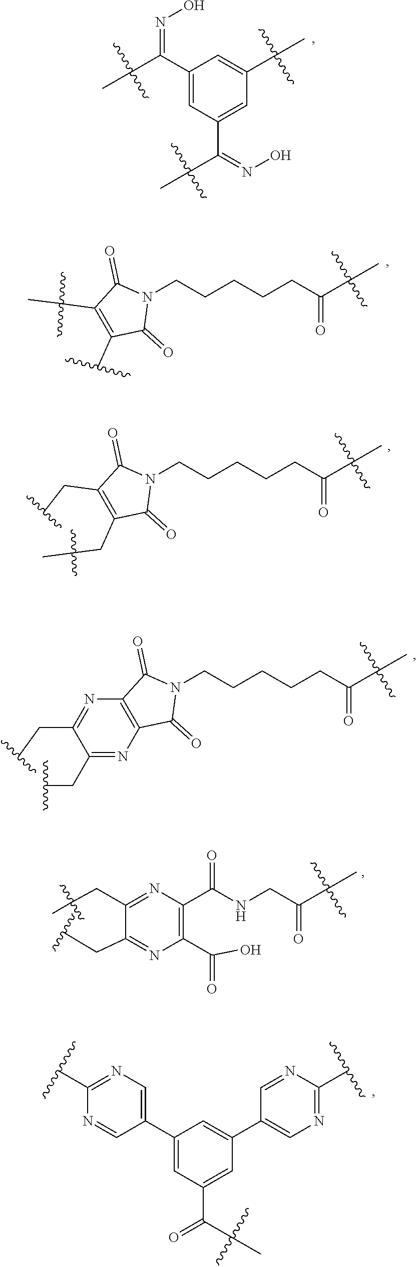 [SMILES: */C(=N/O)c1cc(*)cc(/C(*)=N\O)c1 ; *C(=O)CCCCCN1C(=O)C(*)=C(*)C1=O ; *CC1=C(C*)C(=O)N(CCCCCC(*)=O)C1=O ; *Cc1nc2c(nc1C*)C(=O)N(CCCCCC(*)=O)C2=O ; *Cc1nc(C(=O)O)c(C(=O)NCC(*)=O)nc1C* ; *C(=O)c1cc(-c2cnc(*)nc2)cc(-c2cnc(*)nc2)c1]

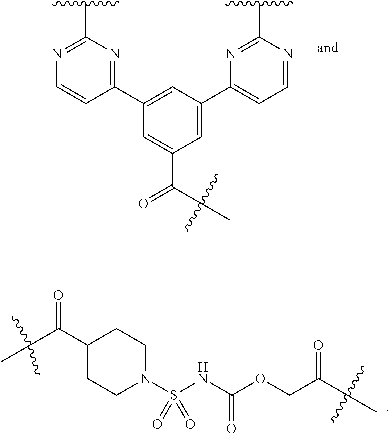 [SMILES: *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the following substituted or unsubstituted structural fragments:

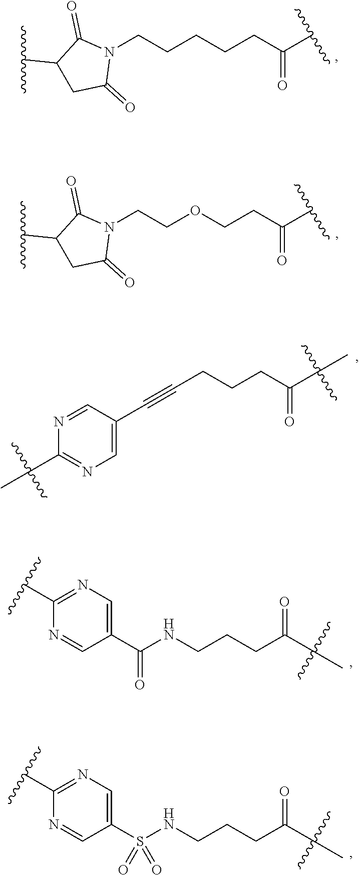 [SMILES: *C(=O)CCCCCN1C(=O)CC(*)C1=O ; *C(=O)CCOCCN1C(=O)CC(*)C1=O ; *C(=O)CCCC#Cc1cnc(*)nc1 ; *C(=O)CCCNC(=O)c1cnc(*)nc1 ; *C(=O)CCCNS(=O)(=O)c1cnc(*)nc1]

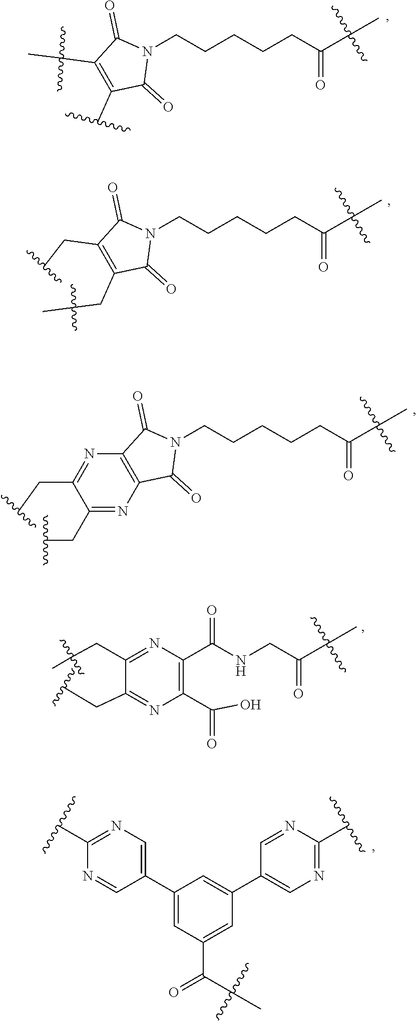 [SMILES: *C(=O)CCCCCN1C(=O)C(*)=C(*)C1=O ; *CC1=C(C*)C(=O)N(CCCCCC(*)=O)C1=O ; *Cc1nc2c(nc1C*)C(=O)N(CCCCCC(*)=O)C2=O ; *Cc1nc(C(=O)O)c(C(=O)NCC(*)=O)nc1C* ; *C(=O)c1cc(-c2cnc(*)nc2)cc(-c2cnc(*)nc2)c1]

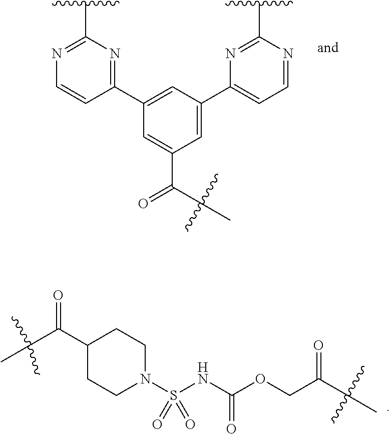 [SMILES: *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the following substituted or unsubstituted structural fragments:

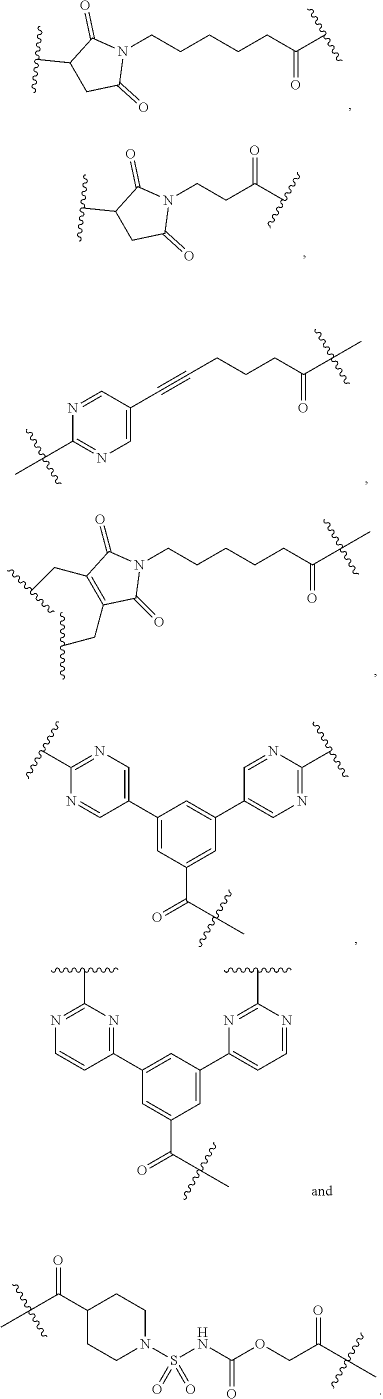

In some embodiments, M is selected from the following substituted or unsubstituted structural fragments:

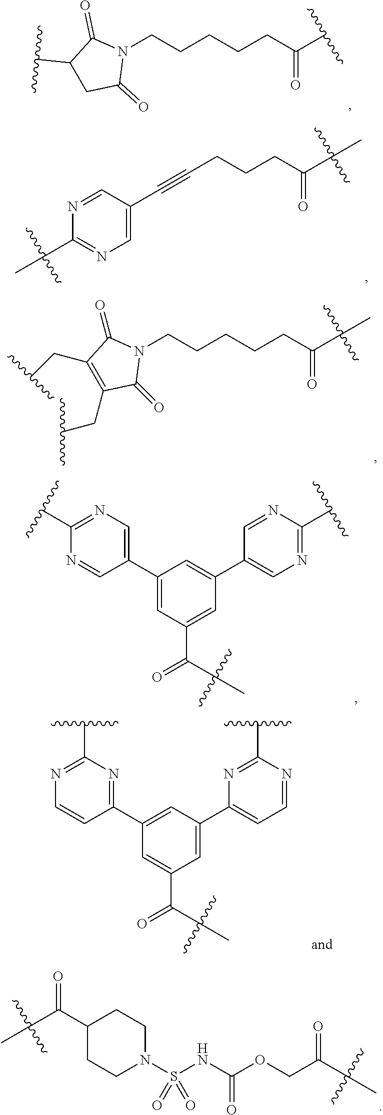 [SMILES: *C(=O)CCCCCN1C(=O)CC(*)C1=O ; *C(=O)CCCC#Cc1cnc(*)nc1 ; *CC1=C(C*)C(=O)N(CCCCCC(*)=O)C1=O ; *C(=O)c1cc(-c2cnc(*)nc2)cc(-c2cnc(*)nc2)c1 ; *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the following substituted or unsubstituted structural fragments:

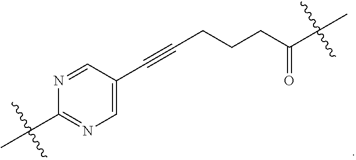 [SMILES: *C(=O)CCCC#Cc1cnc(*)nc1]

In some embodiments, Lg is selected from the group consisting of halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkyl sulfonate group, halogenated C₁₋₆ alkyl sulfonate group, C₁₋₆ alkyl sulfinate group, C₁₋₆ alkylsulfoxide group, halogenated phenoxy, hydroxyl (—OH), sulfhydryl group (—SH), amino (—NH₂), nitro, azido, cyano, alkenyl, alkynyl and alkynyl-containing structural fragment, the halogenated C₁₋₆ alkyl, C₁₋₆ alkylsulfonyl, halogenated C₁₋₆ alkylsulfonyl, halogenated sulfonyl, C₁₋₆ alkyl sulfonate group, halogenated C₁₋₆ alkyl sulfonate group, C₁₋₆ alkyl sulfinate group, C₁₋₆ alkylsulfoxide group, halogenated phenoxy, alkenyl, alkynyl and alkynyl-containing structural fragment are optionally substituted with one or more suitable substituents.

In some embodiments, Lg is selected from the group consisting of halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, hydroxyl (—OH), sulfhydryl group (—SH), amino (—NH₂), nitro, azido, cyano, alkenyl, alkynyl, and alkynyl-containing structural fragment.

In some embodiments, Lg is selected from the group consisting of halogen, substituted or unsubstituted C₁₋₆ alkylsulfonyl, halogenated phenoxy, hydroxyl (—OH), sulfhydryl group (—SH), or amino (—NH₂).

In some embodiments, Lg is selected from the group consisting of halogen, substituted or unsubstituted methylsulfonyl, halogenated phenoxy, hydroxyl (—OH), sulfhydryl group (—SH), or amino (—NH₂).

In some embodiments, Lg is selected from the group consisting of C₁₋₆ alkylsulfonyl.

In some embodiments, Lg is selected from the group consisting of methylsulfonyl.

In some embodiments, M′ is selected from the following structures:

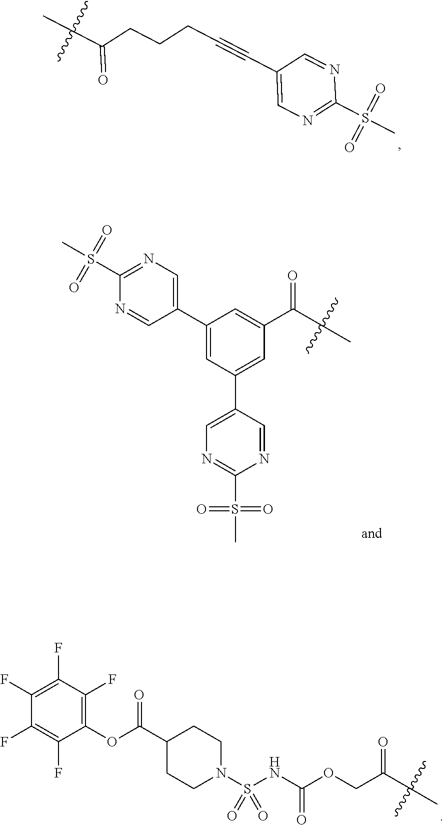

In some embodiments, L is selected from the group consisting of substituted or unsubstituted structural fragments composed of one or more of the following groups: C₁₋₆ alkylene, 6- to 10-membered aryl, 5- to 6-membered heteroaryl, substituted or unsubstituted 9- to 12-membered nitrogen-containing heterocyclyl (e.g., substituted with one or more R′), —N(R′)—, —NH(R′), —N(R′)₂, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Lys(R′), Glu(R′), short peptides composed of amino acids (e.g., Gly-Lys, Asp-Gly-Gly-Phe-Gly (DGGFG, SEQ ID NO: 46), Glu-Gly-Gly-Phe-Gly (EGGFG, SEQ ID NO: 47), Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly (GGFG, SEQ ID NO: 41), Gly-Gly-Val-Ala (GGVA, SEQ ID NO: 42), Gly-Phe-Leu-Gly (GFLG, SEQ ID NO: 43), Glu-Ala-Ala-Ala (EAAA, SEQ ID NO: 44), Gly-Gly-Gly-Gly-Gly (GGGGG, SEQ ID NO: 45)),

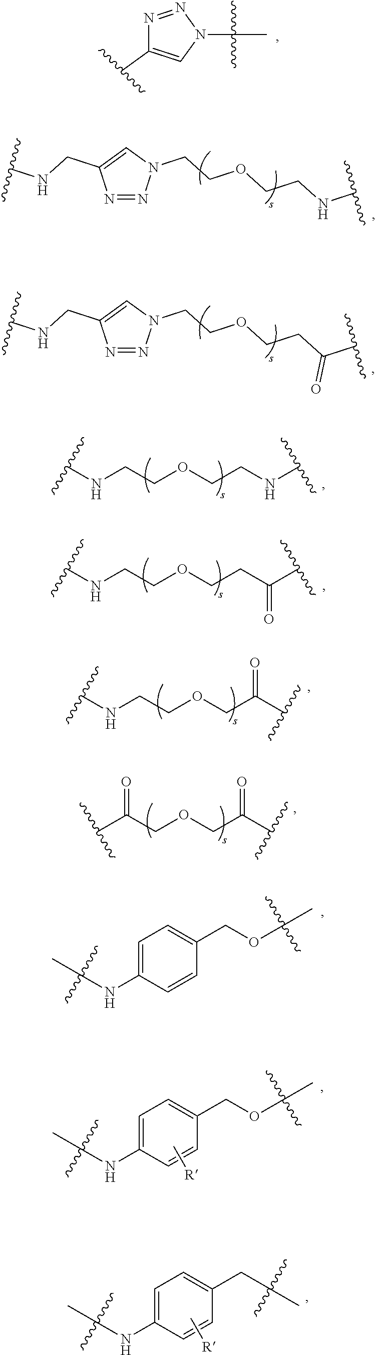

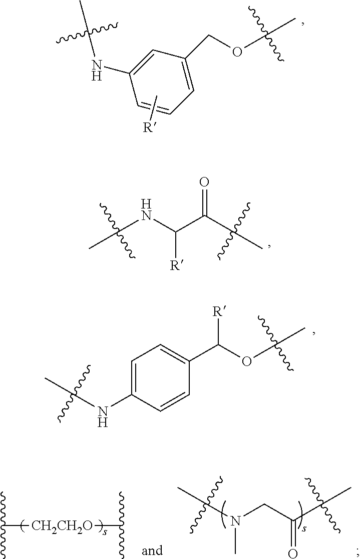

wherein R′ is composed of one or more of the following groups, including but not limited to hydrogen, C₁₋₆ alkyl, C₁₋₆ alkylene, amino, hydroxyl, carboxyl, acyl, —O—, —C₁₋₆ alkylene-CO₂H, —C₁₋₆ alkylene-SO₃H, —SO₃H, —PO₃H₂, —C₁₋₆ alkylene-NHC₁₋₆ alkyl, —C₁₋₆ alkylene-N(C₁₋₆ alkyl)₂, —CH₂N(C₁₋₆ alkyl)-C(═O)C₁₋₆ alkylene-heterocycle, —C₁₋₆ alkylene-heterocycle, —NHC₁₋₆ alkylene-SO₃H,

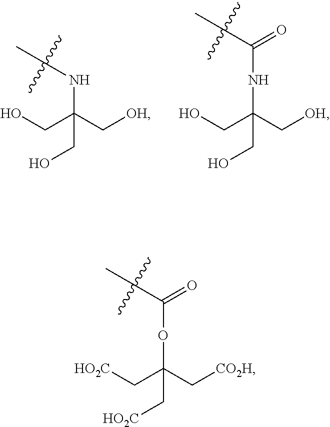 [SMILES: *NC(CO)(CO)CO ; *C(=O)NC(CO)(CO)CO ; *C(=O)OC(CC(=O)O)(CC(=O)O)CC(=O)O]

—CH₂NH—SO₃H, —CH₂N(C₁₋₆ alkyl)-SO₃H, —CH₂NHC₁₋₆ alkylene-SO₃H, —CH₂N(C₁₋₆ alkyl)C₁₋₆ alkylene-SO₃H, —CH₂N(C₁₋₆ alkylene-SO₃H)₂, —CH₂N⁺(C₁₋₆ alkylene-SO₃H)₃, —CH₂N⁺(C₁₋₆ alkyl)₂-C₁₋₆ alkylene-SO₃H, —CH₂N(C₁₋₆ alkyl)-C(═O)C₁₋₆ alkylene-N⁺(C₁₋₆ alkylene-SO₃H)₃, —CH₂NH—C(═O)C₁₋₆ alkylene-N⁺(C₁₋₆ alkylene-SO₃H)₃, —CH₂N(C₁₋₆ alkyl)-C(═O)C₁₋₆ alkylene-N⁺(C₁₋₆ alkyl)₃, —CH₂NH—C(═O)C₁₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₂-CH₂CO₂H, —CH₂N(C₁₋₆ alkyl)-C₁₋₆ alkylene-CO₂H, —CH₂N⁺(C₁₋₆ alkyl)₂-C₁₋₆ alkylene-CO₂H, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—(OCH₂CH₂)ᵣ—OC₁₋₆ alkyl, —(CH₂N(Me)-C(═O))ᵣ—C₁₋₆ alkyl, polyethylene glycol fragment containing 1 to 10 EO units (i.e., —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), NOTA (1,4,7-triazacyclononane-N,N′,N″-triacetic acid residue), —C₁₋₆ alkylene-N(C₁₋₆ alkyl)-DOTA, —C₁₋₆ alkyl-N(C₁₋₆ alkyl)-DOTAGA, or —C₁₋₆ alkyl-N(C₁₋₆ alkyl)-NOTA, wherein r is an integer selected from 1 to 20; s is an integer selected from 1 to 20.

In some embodiments, “—NOTA” refers to

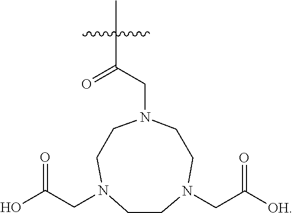 [SMILES: *C(=O)CN1CCN(CC(=O)O)CCN(CC(=O)O)CC1]

In some embodiments, “-DOTA” refers to

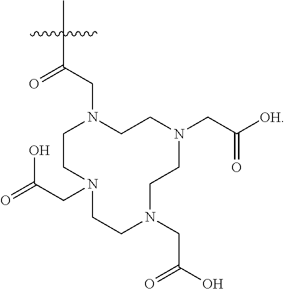 [SMILES: *C(=O)CN1CCN(CC(=O)O)CCN(CC(=O)O)CCN(CC(=O)O)CC1]

In some embodiments, “-DOTAGA” refers to

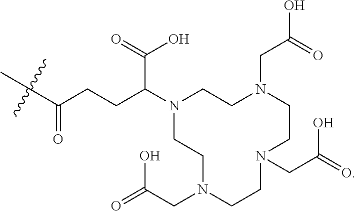 [SMILES: *C(=O)CCC(C(=O)O)N1CCN(CC(=O)O)CCN(CC(=O)O)CCN(CC(=O)O)CC1]

In some embodiments, Lys(R′) refers to the following structure

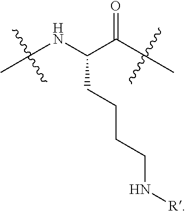

In some embodiments, Glu(R′) refers to the following structure

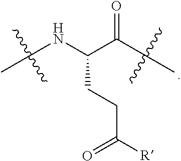

In some embodiments, r is an integer selected from 1 to 15, such as an integer of 1 to 12, 3 to 12, 1 to 10, 1 to 8, 3 to 8, 1 to 6, 1 to 4, 1 to 2, such as 1, 2, 3, 4, 5, 6, 7, 8, 9, 10, 11, 12, 13, 14, 15, 16, 17, 18, 19 or 20.

In some embodiments, s is an integer selected from 1 to 15, such as an integer of 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.

In some embodiments, s is denoted as n.

In some embodiments, L is selected from the group consisting of substituted or unsubstituted structural fragments composed of one or more (e.g., 1, 2, 3, 4, 5, 6, or 7) of the following groups: C₁₋₆ alkylene, —N(R′)—, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Lys(R′), Glu(R′), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly Gly-Gly-Val-Ala, Gly-Phe-Leu-Gly, Glu-Ala-Ala-Ala, Gly-Gly-Gly-Gly-Gly),

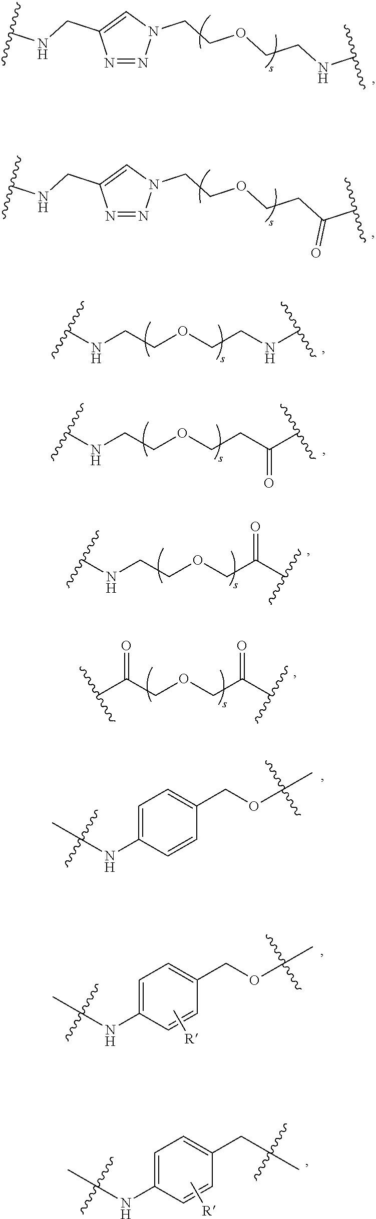

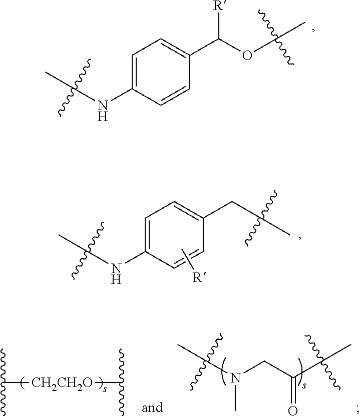

wherein R′ is composed of one or more (e.g., 1, 2, 3, 4, 5, 6 or 7) of the following groups, including but not limited to hydrogen, —C₁₋₆ alkylene-NHC₁₋₆ alkyl, —C₁₋₆ alkylene-N(C₁₋₆ alkyl)₂, DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), NOTA (1,4,7-triazacyclononane-N,N′,N″-triacetic acid residue), —NHC₁₋₆ alkylene-SO₃H, —C₁₋₆ alkylene-N(C₁₋₆ alkyl)-DOTA, —C₁₋₆ alkyl-N(C₁₋₆ alkyl)-DOTAGA, or —C₁₋₆ alkyl-N(C₁₋₆ alkyl)-NOTA, wherein s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.

In some embodiments, L is selected from the group consisting of substituted or unsubstituted structural fragments composed of one or more (e.g., 1, 2, 3, 4, 5, 6, or 7) of the following groups: C₁₋₆ alkylene, —N(R′)—, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Lys(R′), short peptides consisting of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala Gly-Phe-Leu-Gly, Glu-Ala-Ala-Ala, Gly-Gly-Gly-Gly-Gly),

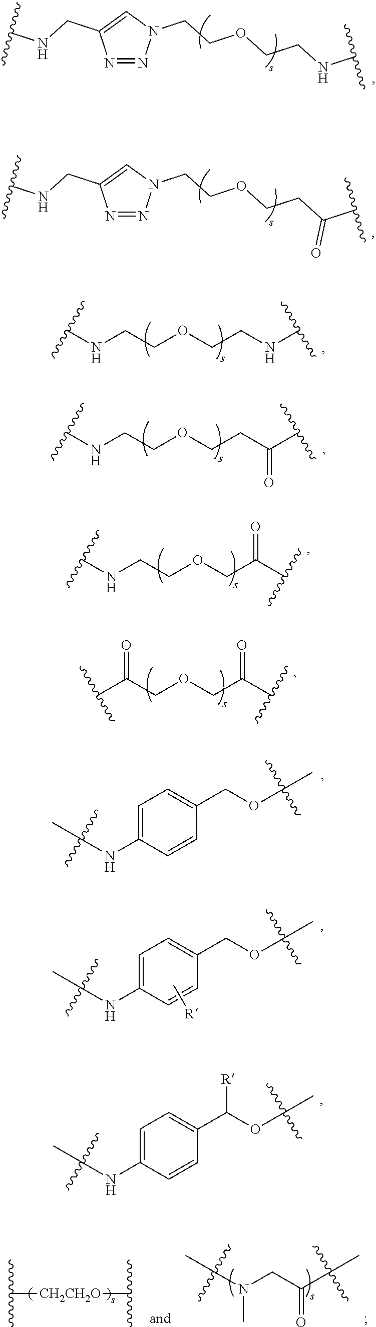

wherein R′ is composed of one or more (e.g., 1, 2, 3, 4, 5, 6 or 7) of the following groups, including but not limited to hydrogen, —C₁₋₆ alkylene-NHC₁₋₆ alkyl, —C₁₋₆ alkylene-N(C₁₋₆ alkyl)₂, DOTA (1,4,7,10-tetraazacyclododecane-1,4,7,10-tetraacetic acid residue), DOTAGA (1,4,7,10-tetraazacyclododecane-1,4, 7,10-tetraacetic acid-α-propionyl), or NOTA (1,4,7-triazacyclononane-N,N′,N″-triacetic acid residue), wherein s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.

In some embodiments, L is selected from the group consisting of substituted or unsubstituted structural fragments composed of one or more of the following: C₁₋₆ alkylene, 6- to 10-membered aryl, 5- to 6-membered heteroaryl, 9- to 12-membered nitrogen-containing heterocyclyl, —N(R′)—, —NH(R′), —N(R′)₂, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Lys(R′), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

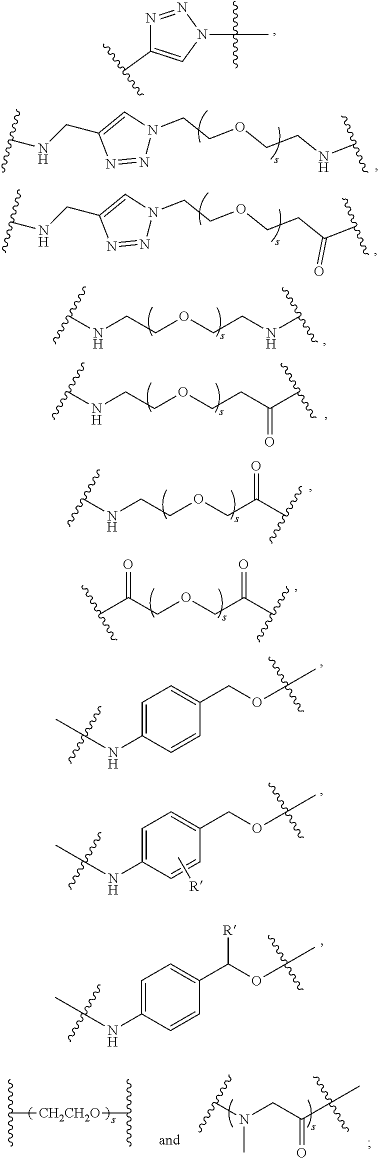

wherein R′ is composed of one or more of the following groups, including but not limited to hydrogen, C₁₋₆ alkyl, C₁₋₆ alkylene, amino, hydroxyl, carboxyl, acyl, —O—, —C₁₋₆ alkylene-CO₂H, —C₁₋₆ alkylene-SO₃H, —SO₃H, —PO₃H₂, —C₁₋₆ alkylene-NHC₁₋₆ alkyl, —C₁₋₆ alkylene-N(C₁₋₆ alkyl)₂, —CH₂N(C₁₋₆ alkyl)-C(═O)C₁₋₆ alkylene-heterocycle, —CH₂NH—SO₃H, —CH₂N(C₁₋₆ alkyl)-SO₃H, —CH₂NHC₁₋₆ alkylene-SO₃H, —CH₂N(C₁₋₆ alkyl)C₁₋₆ alkylene-SO₃H, —CH₂N(C₁₋₆ alkylene-SO₃H)₂, —CH₂N⁺(C₁₋₆ alkylene-SO₃H)₃, —CH₂N⁺(C₁₋₆ alkyl)₂-C₁₋₆ alkylene-SO₃H, —CH₂N(C₁₋₆ alkyl)-C(═O)C₁₋₆ alkylene-N⁺(C₁₋₆ alkylene-SO₃H)₃, —CH₂NH—C(═O)C₁₋₆ alkylene-N⁺(C₁₋₆ alkylene-SO₃H)₃, —CH₂N(C₁₋₆ alkyl)-C(═O)C₁₋₆ alkylene-N⁺(C₁₋₆ alkyl)₃, —CH₂NH—C(═O)C₁₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₂-CH₂CO₂H, —CH₂N(C₁₋₆ alkyl)-C₁₋₆ alkylene-CO₂H, —CH₂N⁺(C₁₋₆ alkyl)₂-C₁₋₆ alkylene-CO₂H, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—(OCH₂CH₂)ᵣ—OC₁₋₆ alkyl, —(CH₂N(Me)-C(═O))ᵣ—C₁₋₆ alkyl, polyethylene glycol fragment containing 1 to 10 EO units (i.e. —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), or NOTA (1,4,7-triazacyclononane-N,N′,N″-triacetic acid residue), wherein r is an integer selected from 1 to 20; s is an integer selected from 1 to 20.

In some embodiments, L is selected from the group consisting of substituted or unsubstituted structural fragments composed of one or more of the following groups: C₁₋₆ alkylene, 6- to 10-membered aryl, 5- to 6-membered heteroaryl, 9- to 12-membered nitrogen-containing heterocyclyl, —N(R′)—, —NH(R′), —N(R′)₂, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

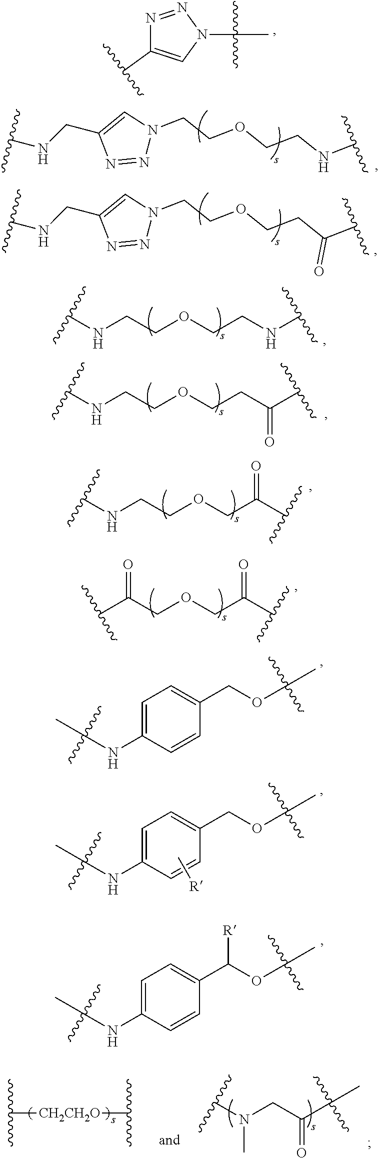

wherein R′ represents hydrogen, C₁₋₆ alkyl, —C₁₋₆ alkylene-CO₂H, —C₁₋₆ alkylene-SO₃H, —SO₃H, —PO₃H₂, —C₁₋₆ alkylene-NHC₁₋₆ alkyl, —C₁₋₆ alkylene-N(C₁₋₆ alkyl)₂, —CH₂N(C₁₋₆ alkyl)-C(═O)C₁₋₆ alkylene-heterocycle, —CH₂NH—SO₃H, —CH₂N(C₁₋₆ alkyl)-SO₃H, —CH₂NHC₁₋₆ alkylene-SO₃H, —CH₂N(C₁₋₆ alkyl)C₁₋₆ alkylene-SO₃H, —CH₂N(C₁₋₆ alkylene-SO₃H)₂, —CH₂N⁺(C₁₋₆ alkylene-SO₃H)₃, —CH₂N⁺(C₁₋₆ alkyl)₂-C₁₋₆ alkylene-SO₃H, —CH₂N(C₁₋₆ alkyl)-C(═O)C₁₋₆ alkylene-N⁺(C₁₋₆ alkylene-SO₃H)₃, —CH₂NH—C(═O)C₁₋₆ alkylene-N⁺(C₁₋₆ alkylene-SO₃H)₃, —CH₂N(C₁₋₆ alkyl)-C(═O)C₁₋₆ alkylene-N⁺(C₁₋₆ alkyl)₃, —CH₂NH—C(═O)C₁₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₂-CH₂CO₂H, —CH₂N(C₁₋₆ alkyl)-C₁₋₆ alkylene-CO₂H, —CH₂N⁺(C₁₋₆ alkyl)₂-C₁₋₆ alkylene-CO₂H, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—(OCH₂CH₂)ᵣ—OC₁₋₆ alkyl, —(CH₂N(Me)-C(═O))ᵣ—C₁₋₆ alkyl, or polyethylene glycol fragment containing 1 to 10 EO units (i.e. —(CH₂CH₂O)ᵣ—C₁₋₆ alkyl), wherein r is an integer selected from 1 to 20; s is an integer selected from 1 to 20.

In some embodiments, L is selected from the group consisting of substituted or unsubstituted structural fragments composed of one or more of the following groups: C₁₋₆ alkylene, 6- to 10-membered aryl, 5- to 6-membered heteroaryl, —N(R′)—, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Lys(R′), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

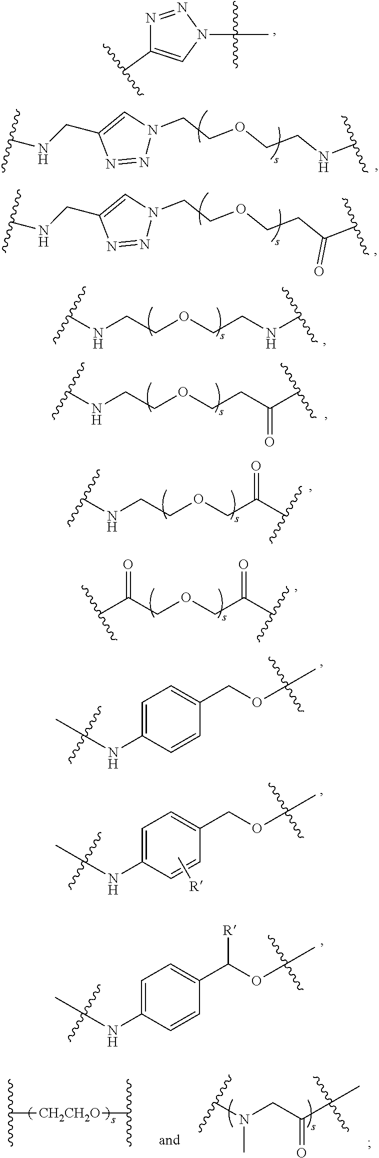

wherein R′ is composed of one or more of the following groups, including but not limited to hydrogen, C₁₋₆ alkyl, C₁₋₆ alkylene, amino, hydroxyl, carboxyl, acyl, —O—, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, polyethylene glycol fragment containing 1 to 10 EO units (e.g., —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), or NOTA (1,4,7-triazacyclononane-N,N′,N″-triacetic acid residue), wherein r is an integer selected from 1 to 20; s is an integer selected from 1 to 20.

In some embodiments, L is selected from the group consisting of substituted or unsubstituted structural fragments composed of one or more of the following groups: C₁₋₆ alkylene, 6- to 10-membered aryl, 5- to 6-membered heteroaryl, —N(R′)—, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

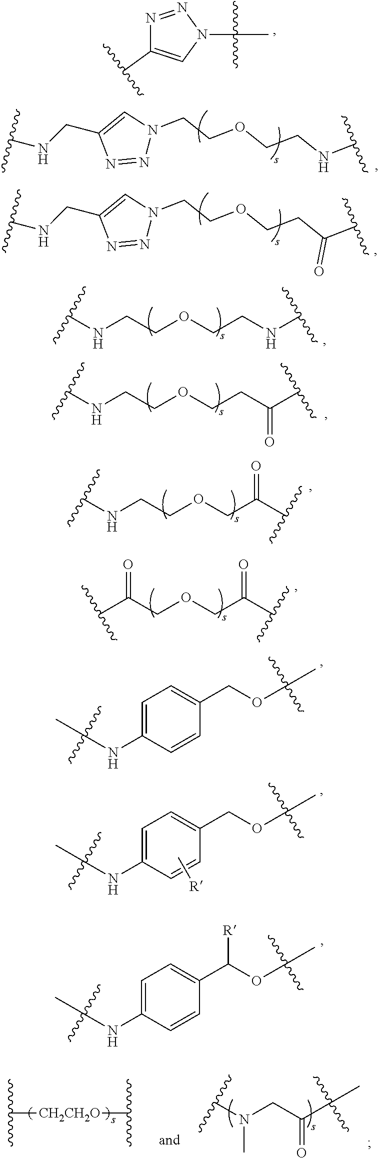

wherein R′ represents hydrogen, C₁₋₆ alkyl, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, or polyethylene glycol fragment containing 1 to 10 EO units (i.e. —(CH₂CH₂O)ᵣ—C₁₋₆ alkyl), wherein r is an integer selected from 1 to 20; s is an integer selected from 1 to 20.

In some embodiments, L is selected from the group consisting of substituted or unsubstituted structural fragments composed of one or more of the following groups: C₁₋₆ alkylene, carbonyl group, 9- to 12-membered nitrogen-containing heterocyclyl, —NH—, Ala-Ala, Ala-Lys, Ala-Pro, Gly-Glu, Gly-Gly, Phe-Lys, Val-Ala, Val-Cit, Val-Lys, Ala-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly,

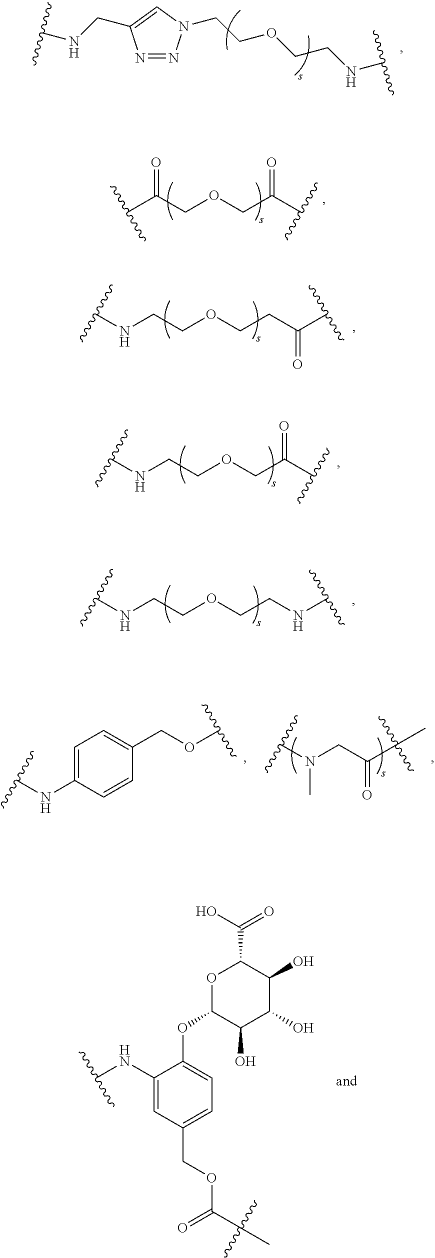

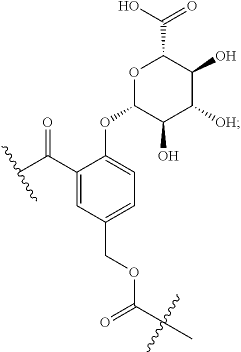

wherein s is an integer selected from 1 to 20.

In some embodiments, L is formed by connecting one or more substituted or unsubstituted structural fragments selected from the following Group I with one or more substituted or unsubstituted structural fragments selected from the following Group II:

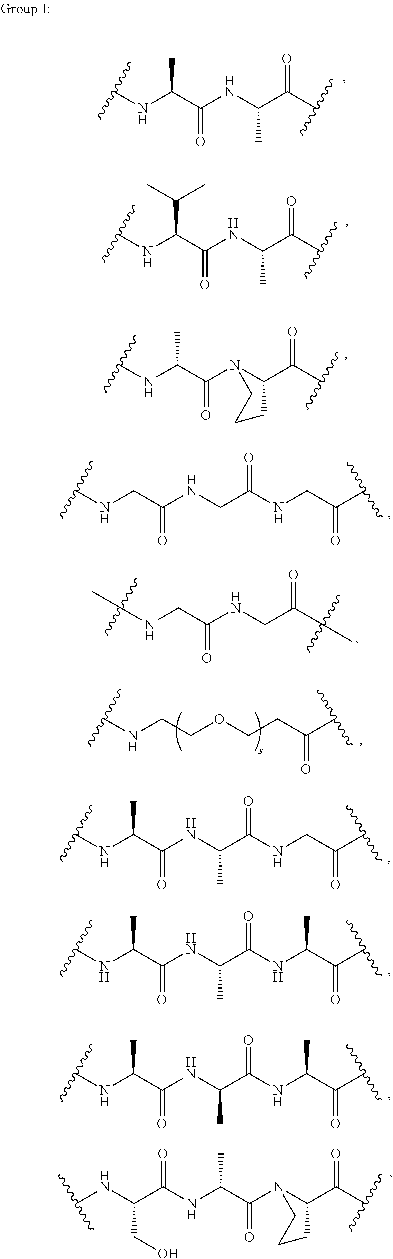

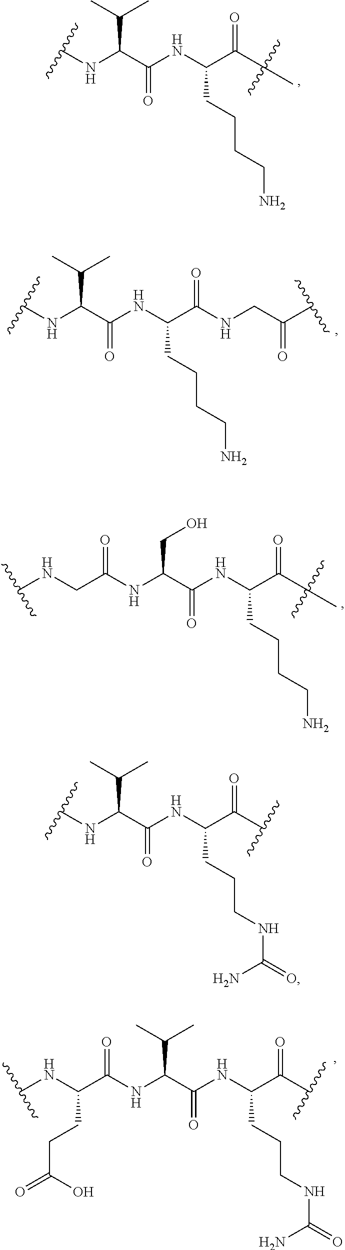

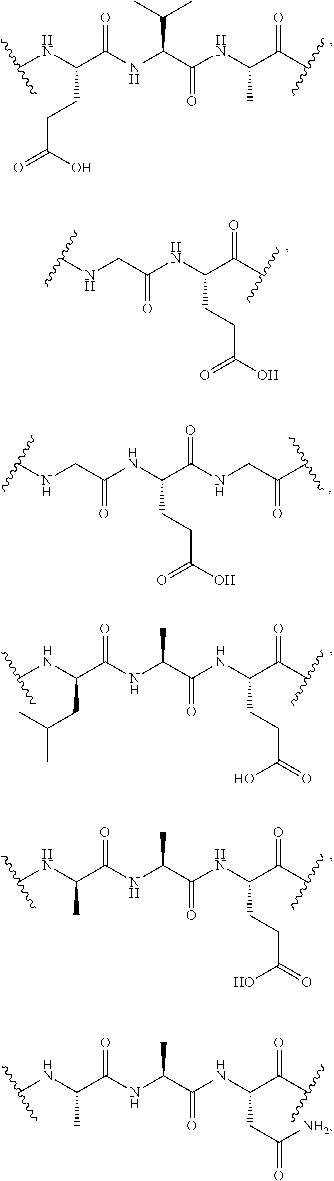

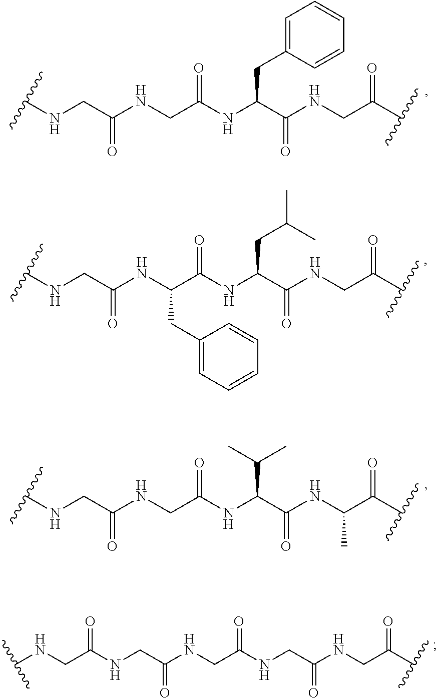

wherein s is an integer selected from 1 to 20;

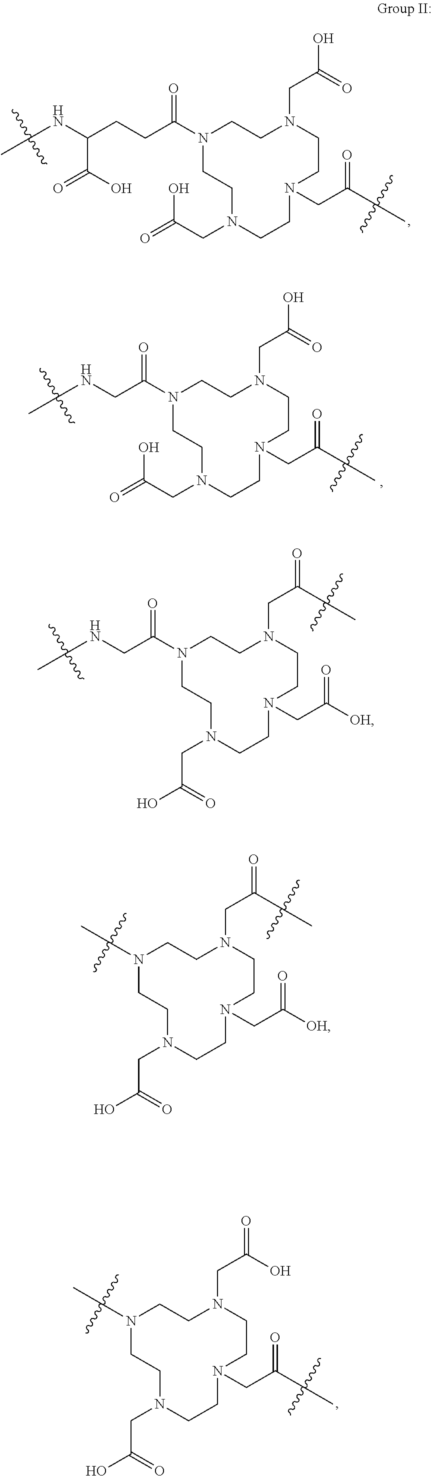 [SMILES: *NC(CCC(=O)N1CCN(CC(=O)O)CCN(CC(*)=O)CCN(CC(=O)O)CC1)C(=O)O ; *NCC(=O)N1CCN(CC(=O)O)CCN(CC(*)=O)CCN(CC(=O)O)CC1 ; *NCC(=O)N1CCN(CC(*)=O)CCN(CC(=O)O)CCN(CC(=O)O)CC1 ; *C(=O)CN1CCN(*)CCN(CC(=O)O)CCN(CC(=O)O)CC1 ; *C(=O)CN1CCN(CC(=O)O)CCN(*)CCN(CC(=O)O)CC1]

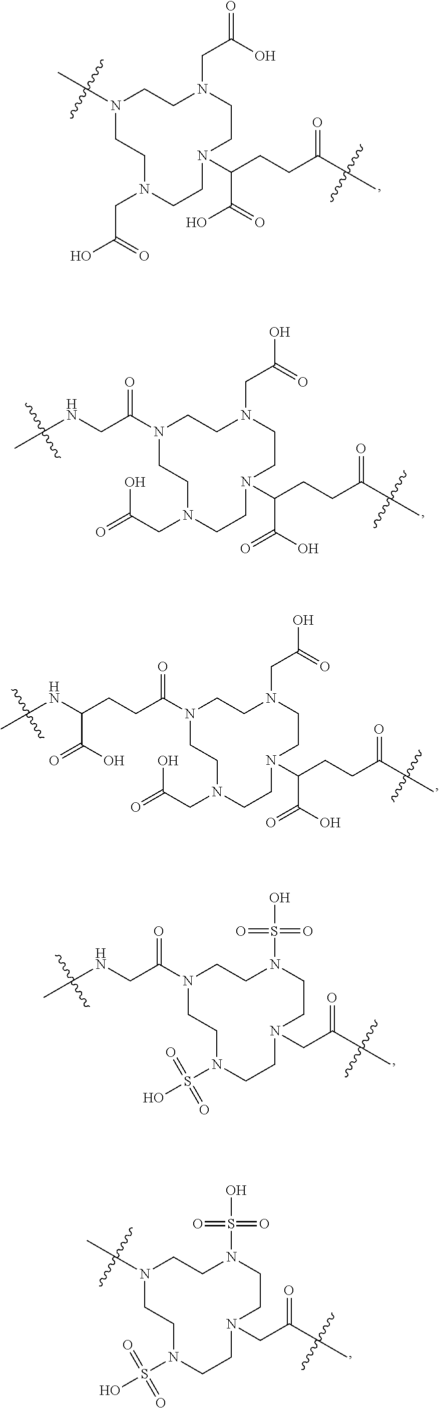 [SMILES: *C(=O)CCC(C(=O)O)N1CCN(CC(=O)O)CCN(*)CCN(CC(=O)O)CC1 ; *NCC(=O)N1CCN(CC(=O)O)CCN(C(CCC(*)=O)C(=O)O)CCN(CC(=O)O)CC1 ; *NC(CCC(=O)N1CCN(CC(=O)O)CCN(C(CCC(*)=O)C(=O)O)CCN(CC(=O)O)CC1)C(=O)O ; *NCC(=O)N1CCN(S(=O)(=O)O)CCN(CC(*)=O)CCN(S(=O)(=O)O)CC1 ; *C(=O)CN1CCN(S(=O)(=O)O)CCN(*)CCN(S(=O)(=O)O)CC1]

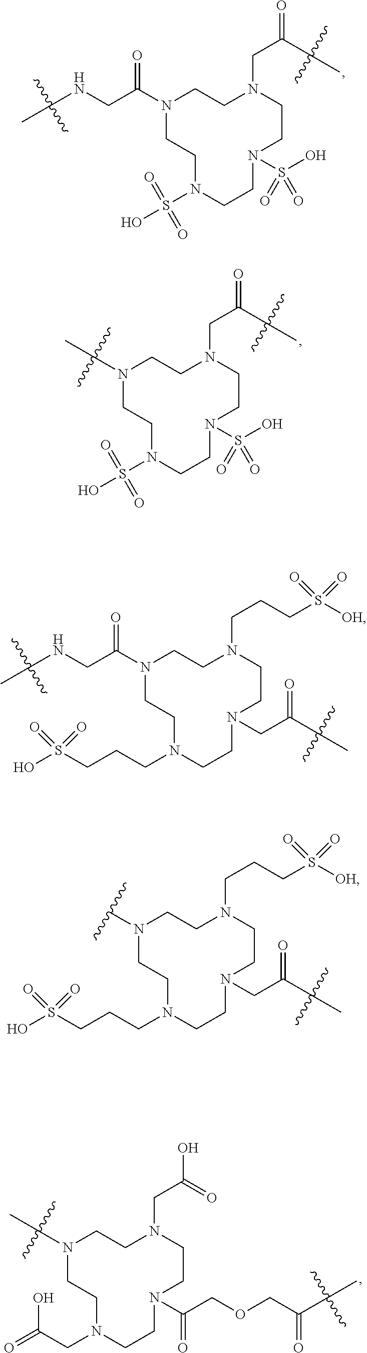 [SMILES: *NCC(=O)N1CCN(CC(*)=O)CCN(S(=O)(=O)O)CCN(S(=O)(=O)O)CC1 ; *C(=O)CN1CCN(*)CCN(S(=O)(=O)O)CCN(S(=O)(=O)O)CC1 ; *NCC(=O)N1CCN(CCCS(=O)(=O)O)CCN(CC(*)=O)CCN(CCCS(=O)(=O)O)CC1 ; *C(=O)CN1CCN(CCCS(=O)(=O)O)CCN(*)CCN(CCCS(=O)(=O)O)CC1 ; *C(=O)COCC(=O)N1CCN(CC(=O)O)CCN(*)CCN(CC(=O)O)CC1]

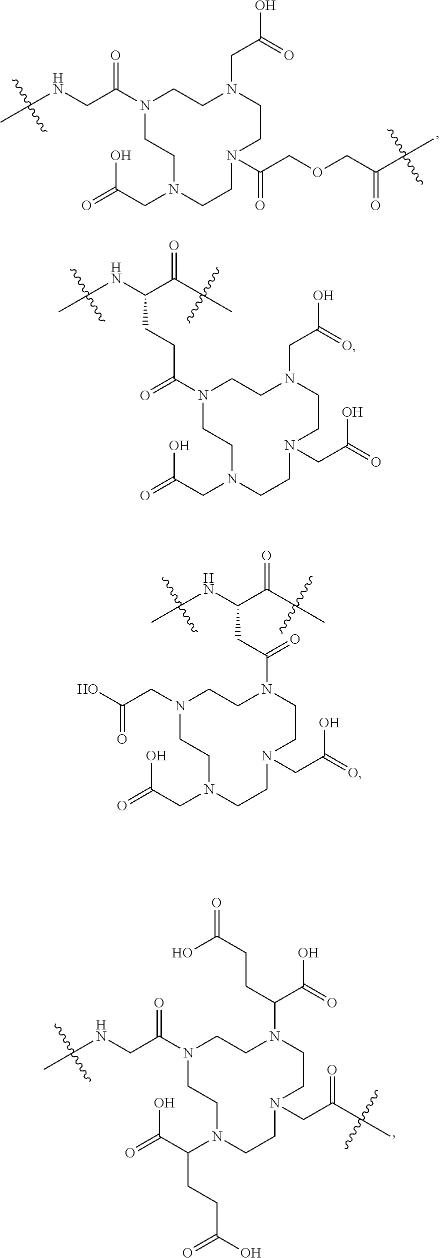 [SMILES: *NCC(=O)N1CCN(CC(=O)O)CCN(C(=O)COCC(*)=O)CCN(CC(=O)O)CC1 ; *N[C@@H](CCC(=O)N1CCN(CC(=O)O)CCN(CC(=O)O)CCN(CC(=O)O)CC1)C(*)=O ; *N[C@@H](CC(=O)N1CCN(CC(=O)O)CCN(CC(=O)O)CCN(CC(=O)O)CC1)C(*)=O ; *NCC(=O)N1CCN(C(CCC(=O)O)C(=O)O)CCN(CC(*)=O)CCN(C(CCC(=O)O)C(=O)O)CC1]

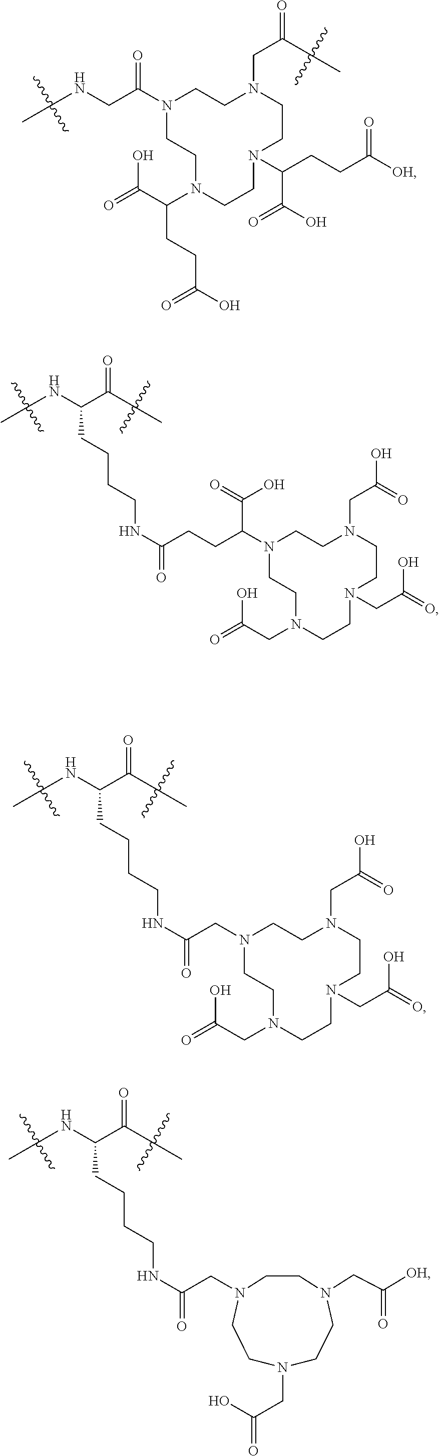 [SMILES: *NCC(=O)N1CCN(CC(*)=O)CCN(C(CCC(=O)O)C(=O)O)CCN(C(CCC(=O)O)C(=O)O)CC1 ; *N[C@@H](CCCCNC(=O)CCC(C(=O)O)N1CCN(CC(=O)O)CCN(CC(=O)O)CCN(CC(=O)O)CC1)C(*)=O ; *N[C@@H](CCCCNC(=O)CN1CCN(CC(=O)O)CCN(CC(=O)O)CCN(CC(=O)O)CC1)C(*)=O ; *N[C@@H](CCCCNC(=O)CN1CCN(CC(=O)O)CCN(CC(=O)O)CC1)C(*)=O]

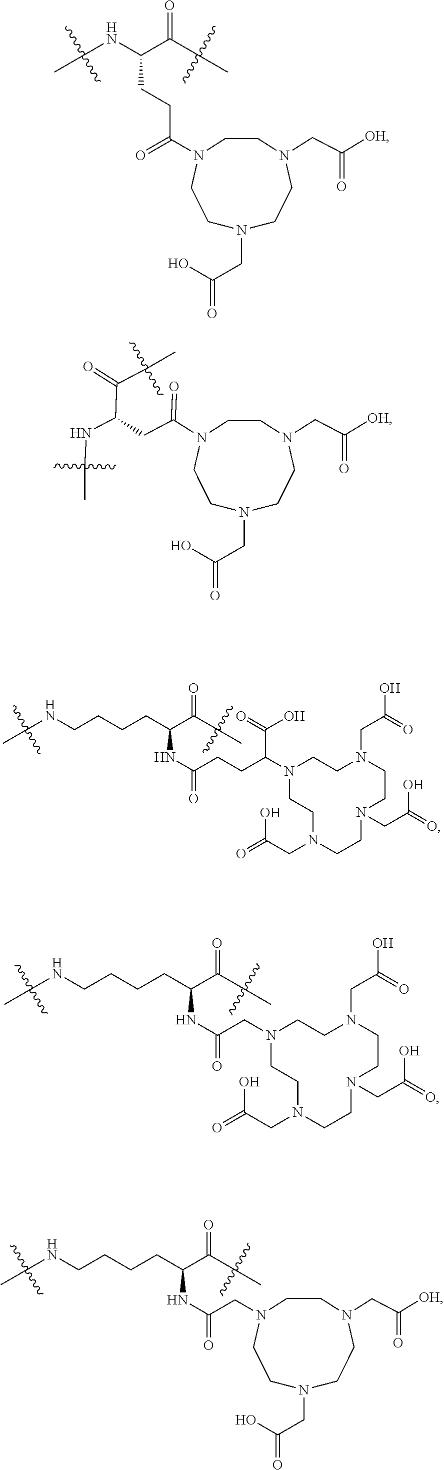 [SMILES: *N[C@@H](CCC(=O)N1CCN(CC(=O)O)CCN(CC(=O)O)CC1)C(*)=O ; *N[C@@H](CC(=O)N1CCN(CC(=O)O)CCN(CC(=O)O)CC1)C(*)=O ; *NCCCC[C@H](NC(=O)CCC(C(=O)O)N1CCN(CC(=O)O)CCN(CC(=O)O)CCN(CC(=O)O)CC1)C(*)=O ; *NCCCC[C@H](NC(=O)CN1CCN(CC(=O)O)CCN(CC(=O)O)CCN(CC(=O)O)CC1)C(*)=O ; *NCCCC[C@H](NC(=O)CN1CCN(CC(=O)O)CCN(CC(=O)O)CC1)C(*)=O]

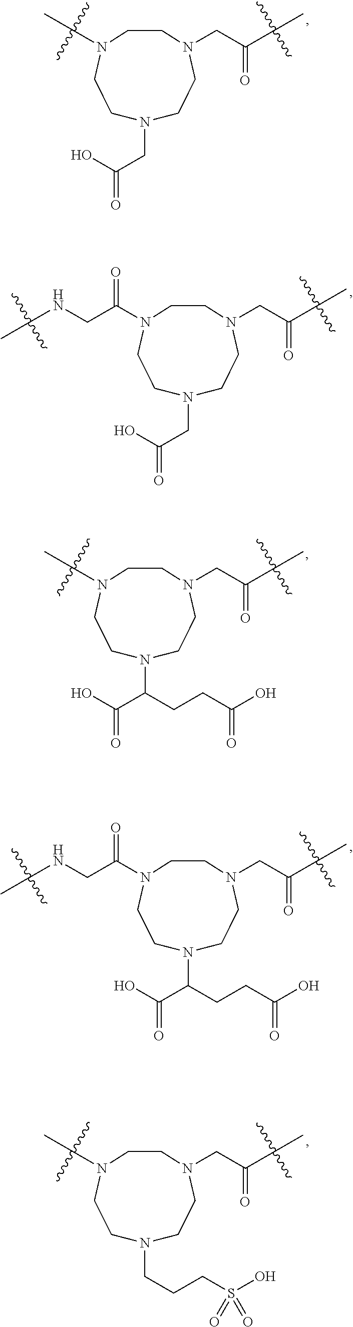 [SMILES: *C(=O)CN1CCN(*)CCN(CC(=O)O)CC1 ; *NCC(=O)N1CCN(CC(*)=O)CCN(CC(=O)O)CC1 ; *C(=O)CN1CCN(*)CCN(C(CCC(=O)O)C(=O)O)CC1 ; *NCC(=O)N1CCN(CC(*)=O)CCN(C(CCC(=O)O)C(=O)O)CC1 ; *C(=O)CN1CCN(*)CCN(CCCS(=O)(=O)O)CC1]

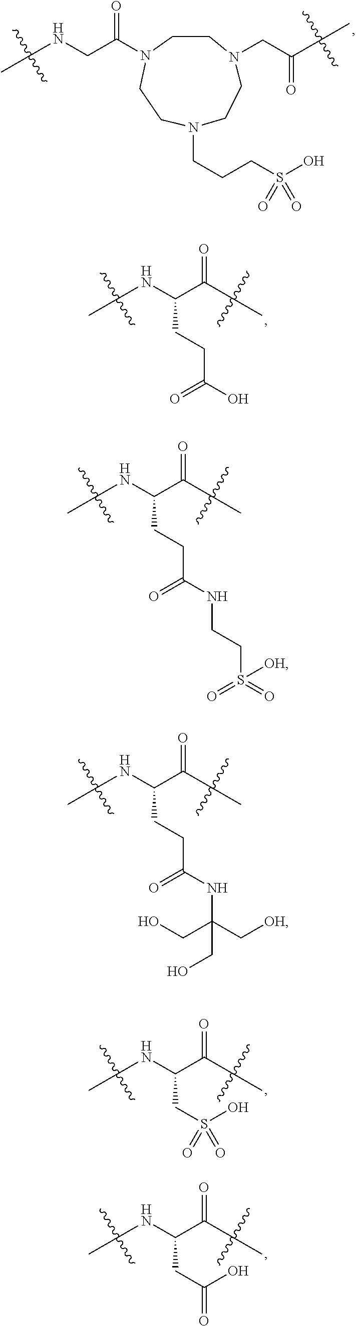

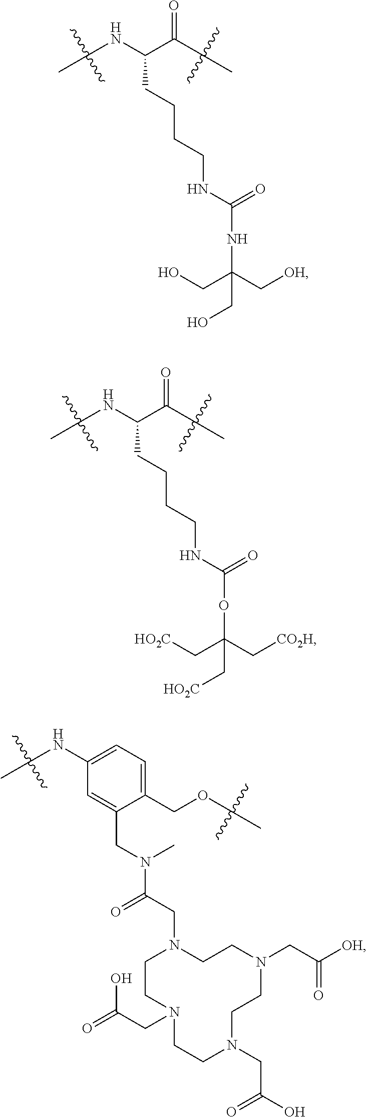 [SMILES: *N[C@@H](CCCCNC(=O)NC(CO)(CO)CO)C(*)=O ; *N[C@@H](CCCCNC(=O)OC(CC(=O)O)(CC(=O)O)CC(=O)O)C(*)=O ; *Nc1ccc(CO*)c(CN(C)C(=O)CN2CCN(CC(=O)O)CCN(CC(=O)O)CCN(CC(=O)O)CC2)c1]

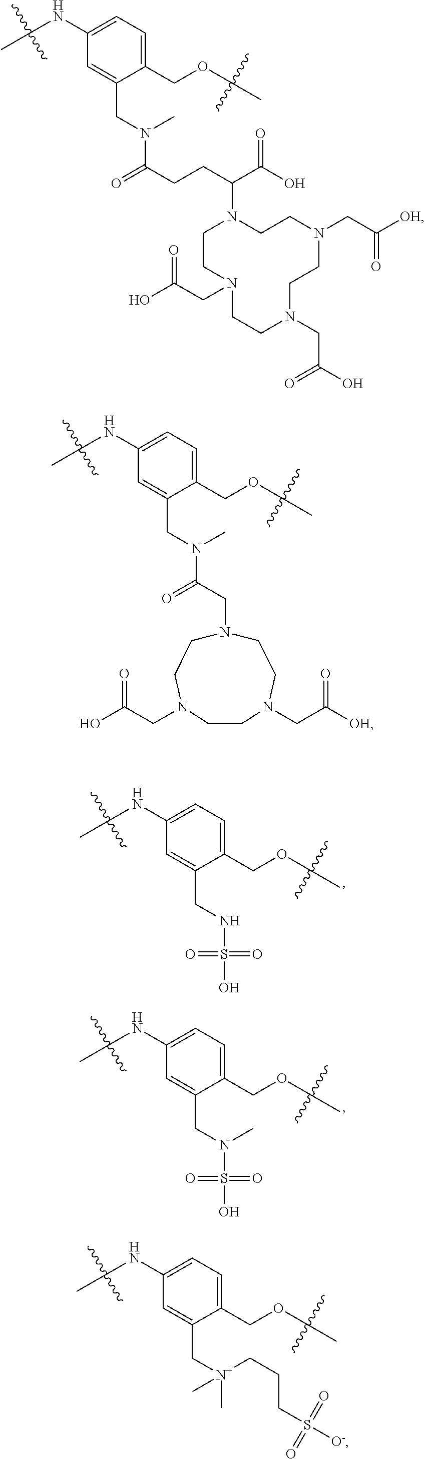

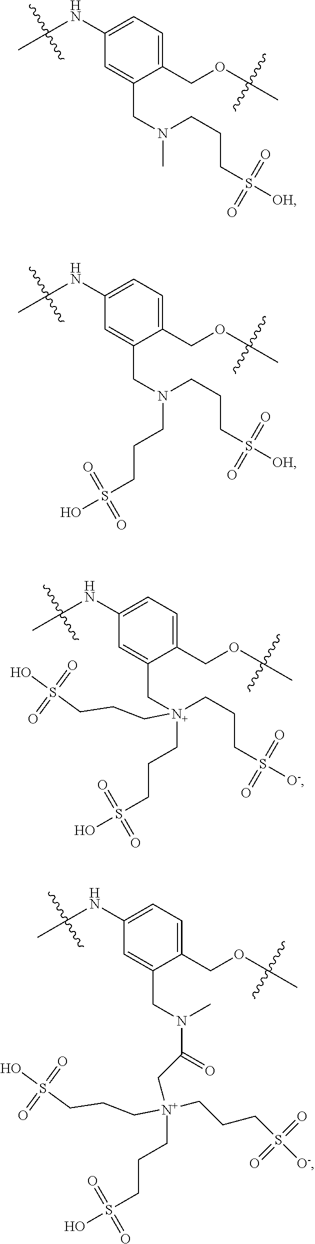

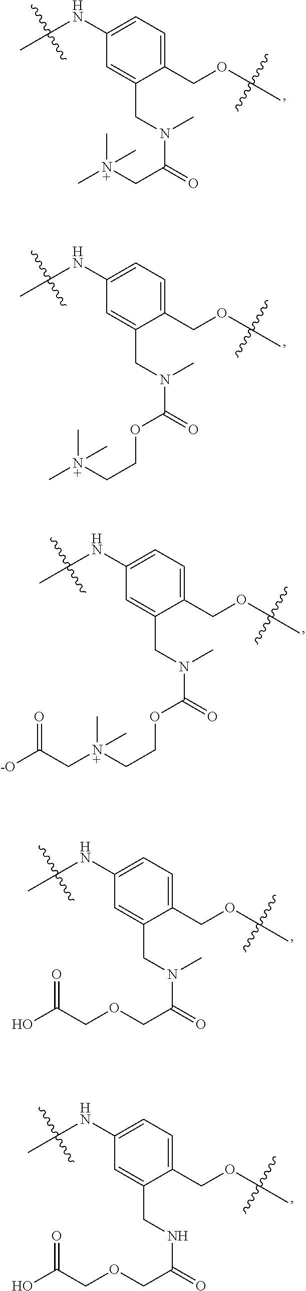

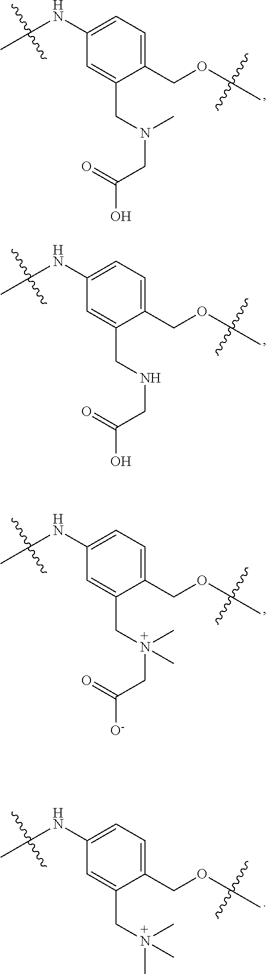

In some embodiments, L is formed by connecting one or more substituted or unsubstituted structural fragments selected from the following Group I with one or more substituted or unsubstituted structural fragments selected from the following Group II:

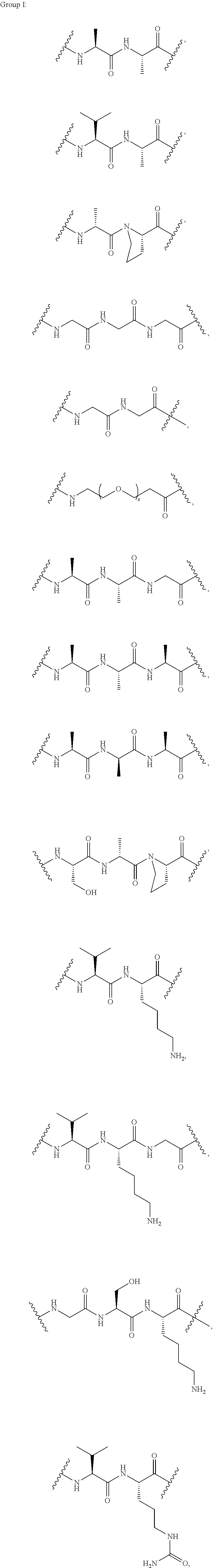

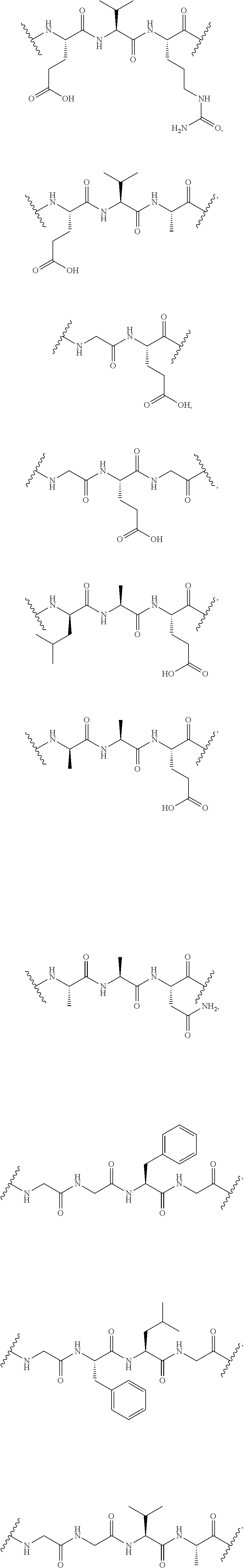

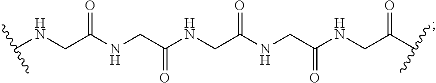

wherein s is an integer selected from 1 to 20;

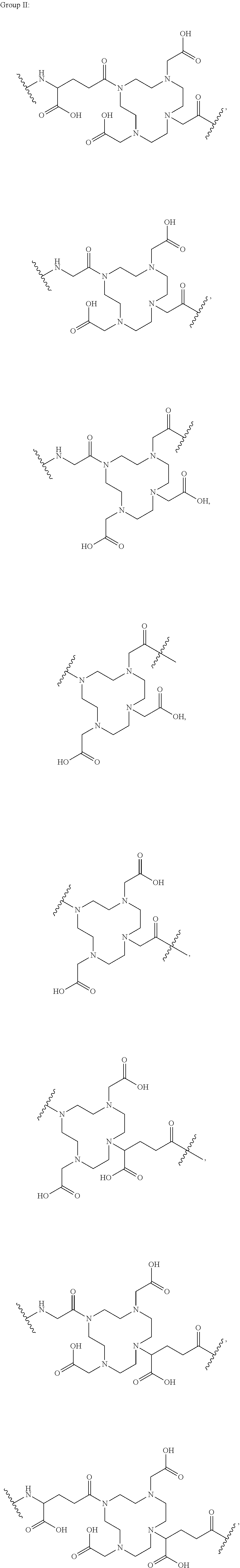

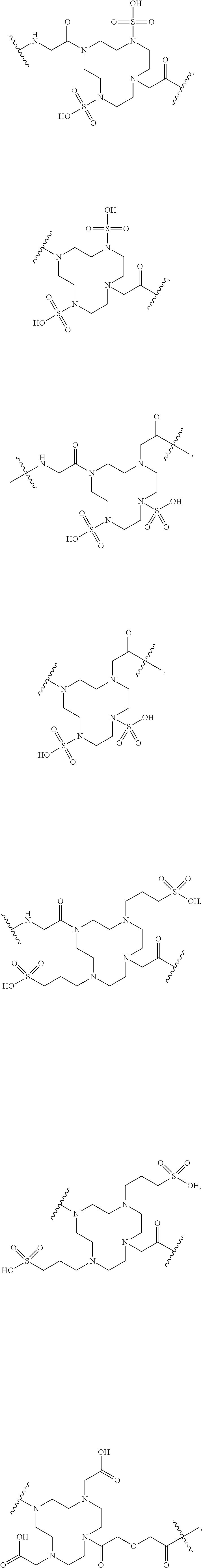

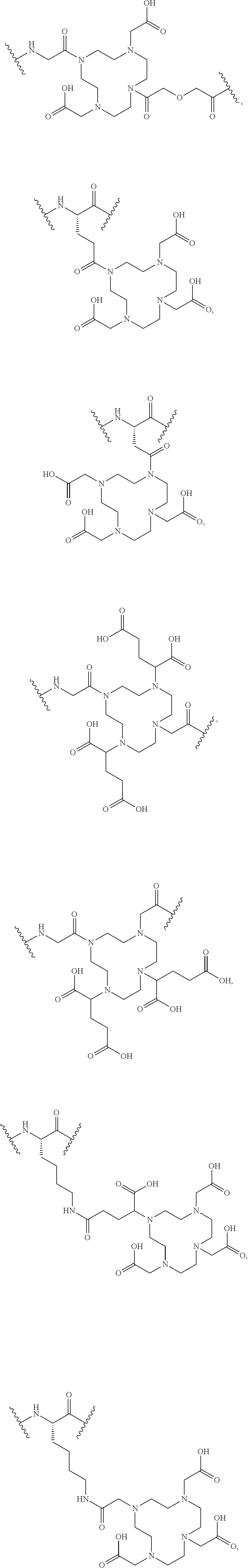

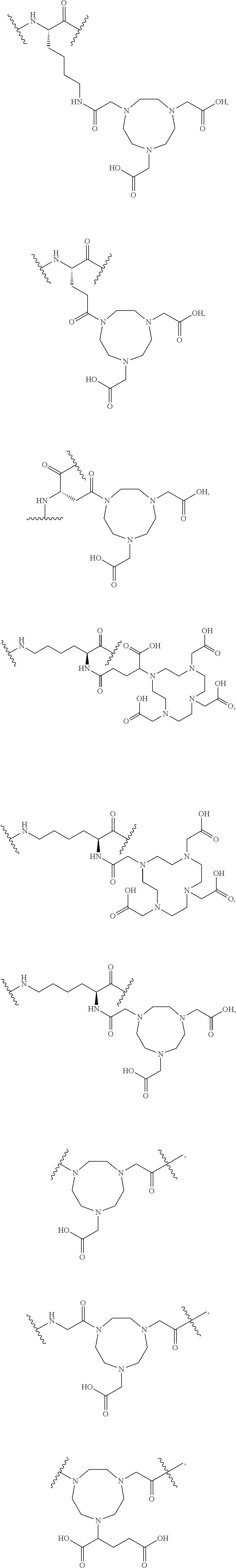

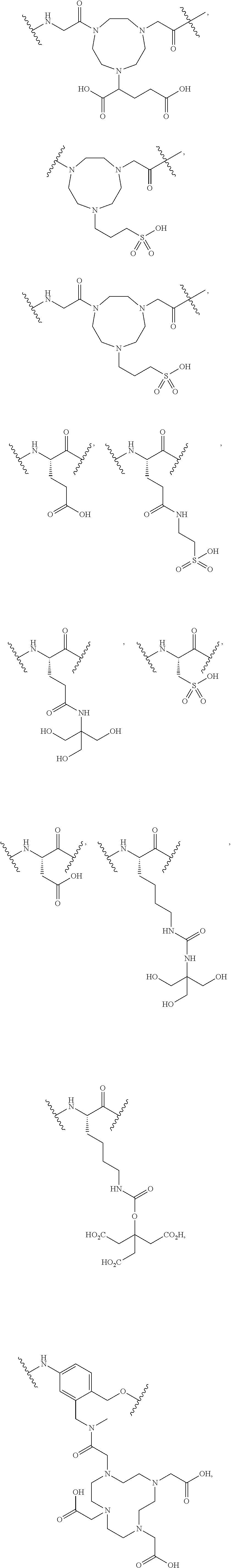

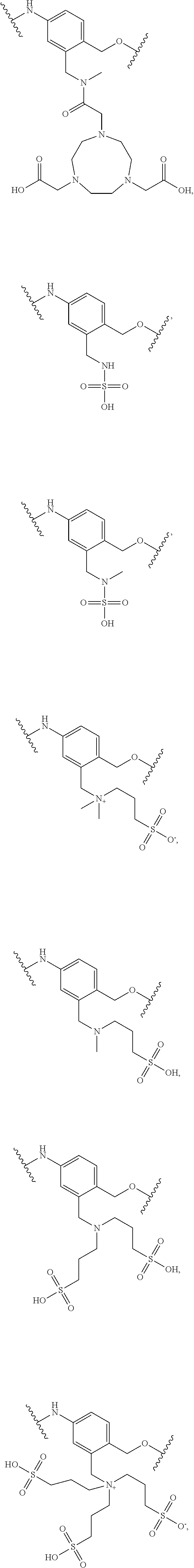

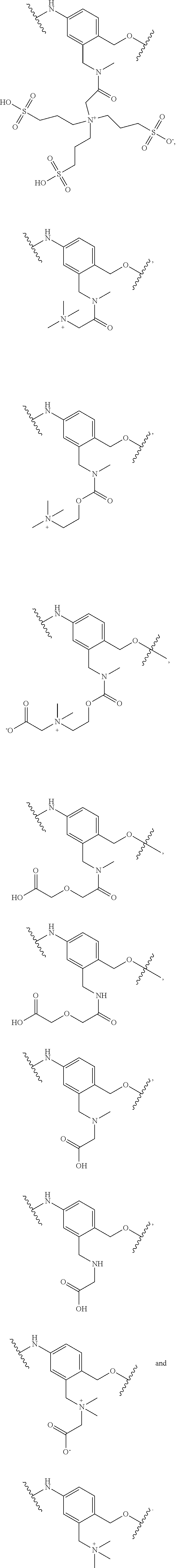

In some embodiments, L is formed by connecting one or more substituted or unsubstituted structural fragments selected from the Group I and one or more substituted or unsubstituted structural fragments selected from the Group II:

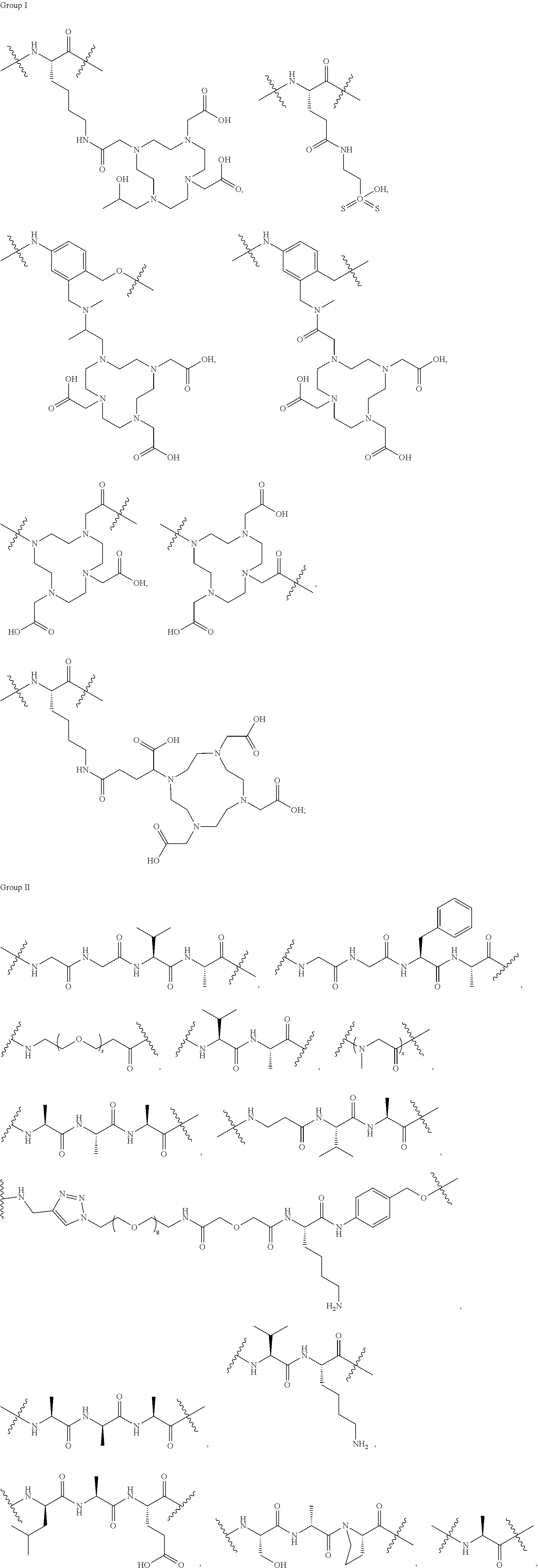

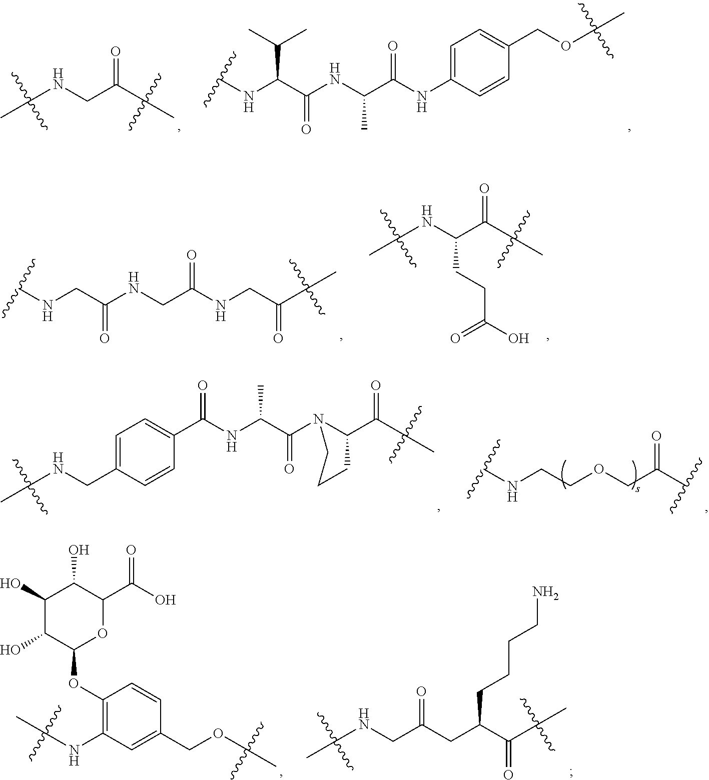

s is an integer selected from of 1 to 20, such as 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.

In some embodiments, Group I consists of

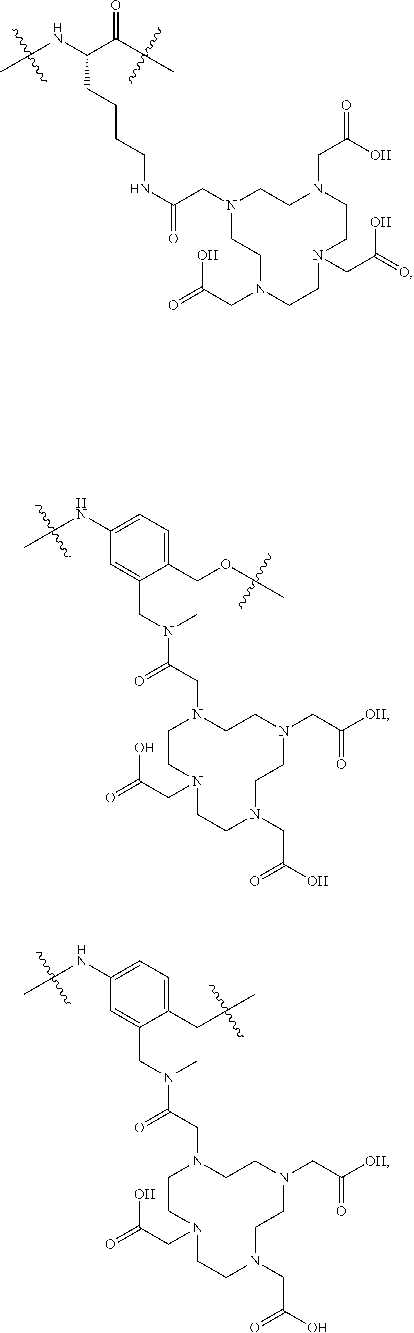 [SMILES: *N[C@@H](CCCCNC(=O)CN1CCN(CC(=O)O)CCN(CC(=O)O)CCN(CC(=O)O)CC1)C(*)=O ; *Nc1ccc(CO*)c(CN(C)C(=O)CN2CCN(CC(=O)O)CCN(CC(=O)O)CCN(CC(=O)O)CC2)c1 ; *Cc1ccc(N*)cc1CN(C)C(=O)CN1CCN(CC(=O)O)CCN(CC(=O)O)CCN(CC(=O)O)CC1]

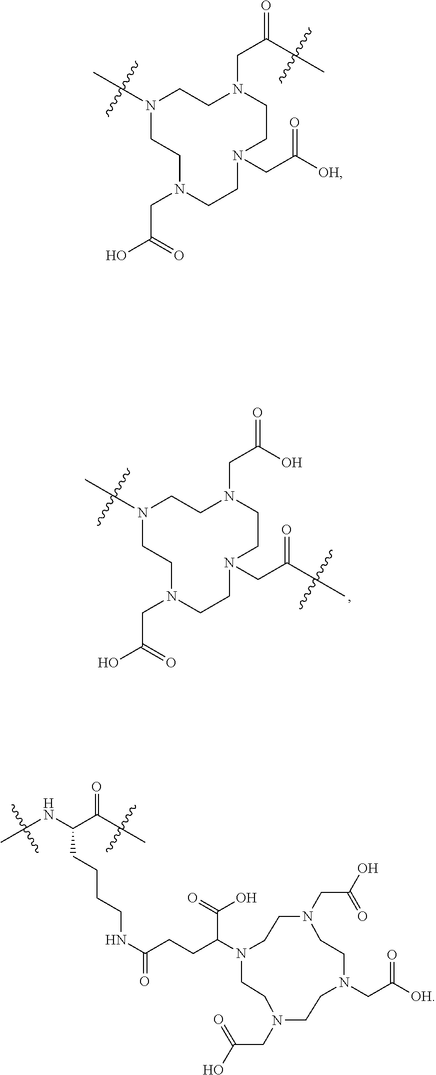 [SMILES: *C(=O)CN1CCN(*)CCN(CC(=O)O)CCN(CC(=O)O)CC1 ; *C(=O)CN1CCN(CC(=O)O)CCN(*)CCN(CC(=O)O)CC1 ; *N[C@@H](CCCCNC(=O)CCC(C(=O)O)N1CCN(CC(=O)O)CCN(CC(=O)O)CCN(CC(=O)O)CC1)C(*)=O]

In some embodiments, Group I consists of

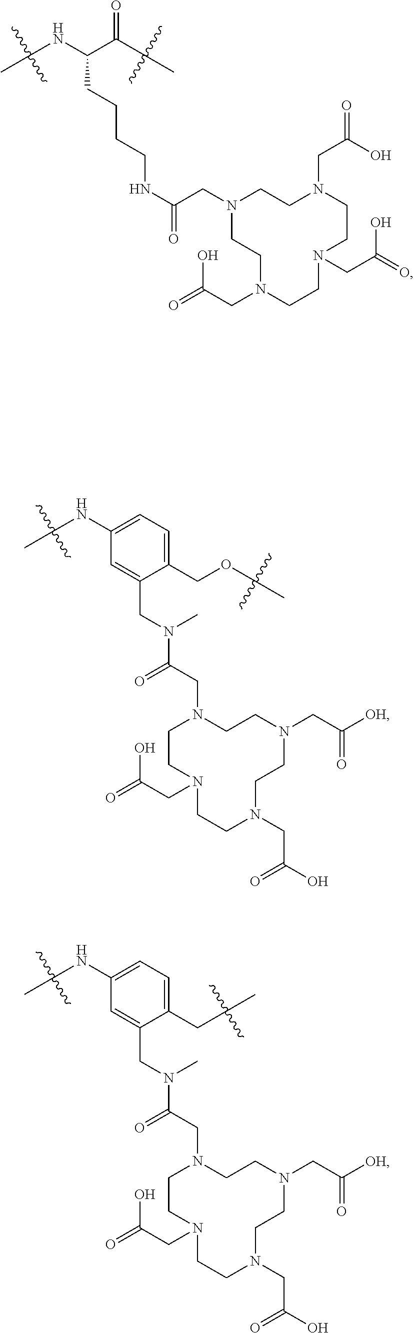 [SMILES: *N[C@@H](CCCCNC(=O)CN1CCN(CC(=O)O)CCN(CC(=O)O)CCN(CC(=O)O)CC1)C(*)=O ; *Nc1ccc(CO*)c(CN(C)C(=O)CN2CCN(CC(=O)O)CCN(CC(=O)O)CCN(CC(=O)O)CC2)c1 ; *Cc1ccc(N*)cc1CN(C)C(=O)CN1CCN(CC(=O)O)CCN(CC(=O)O)CCN(CC(=O)O)CC1]

In some embodiments, Group II consists of

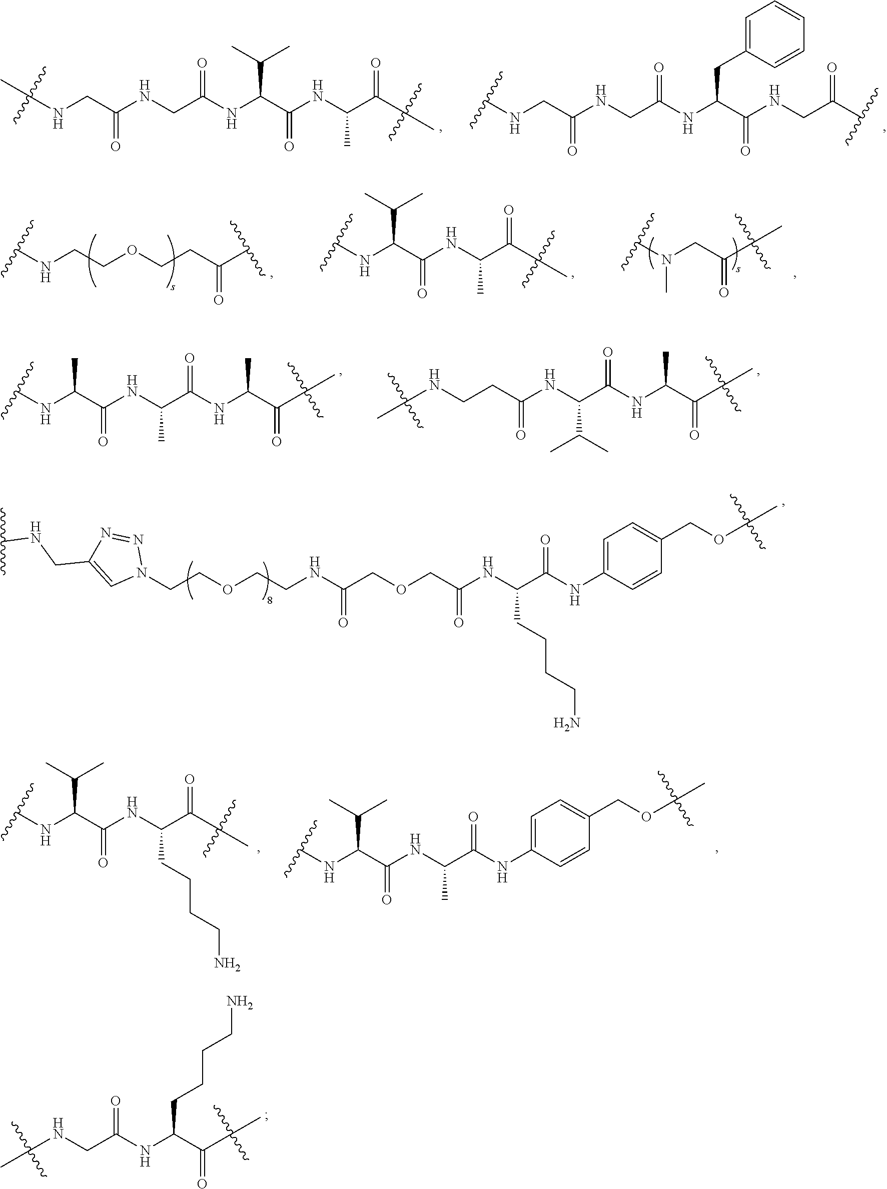

s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.

In some embodiments, L is selected from the group consisting of substituted or unsubstituted structural fragments composed of one or more of the following groups:

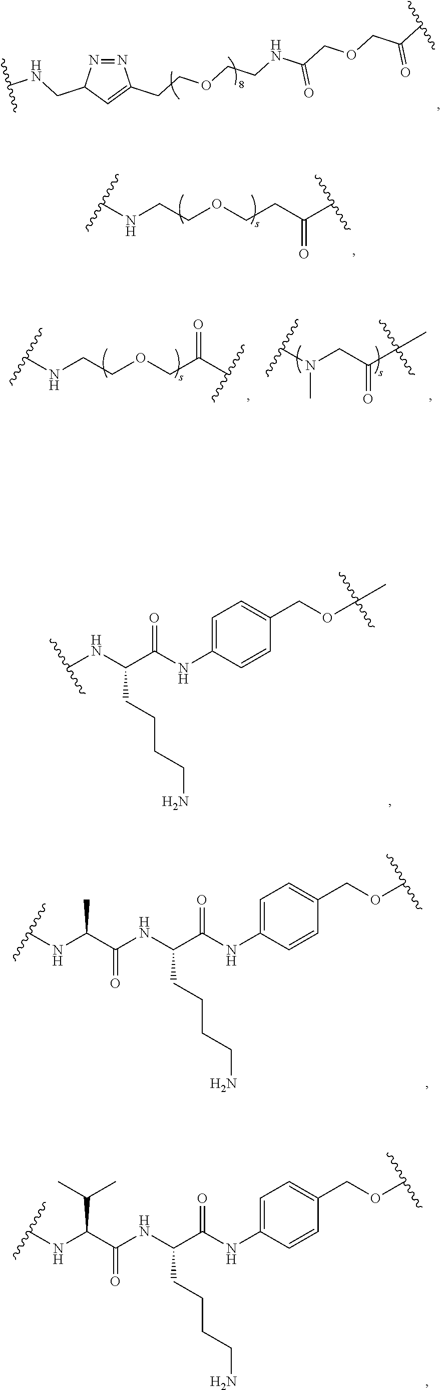

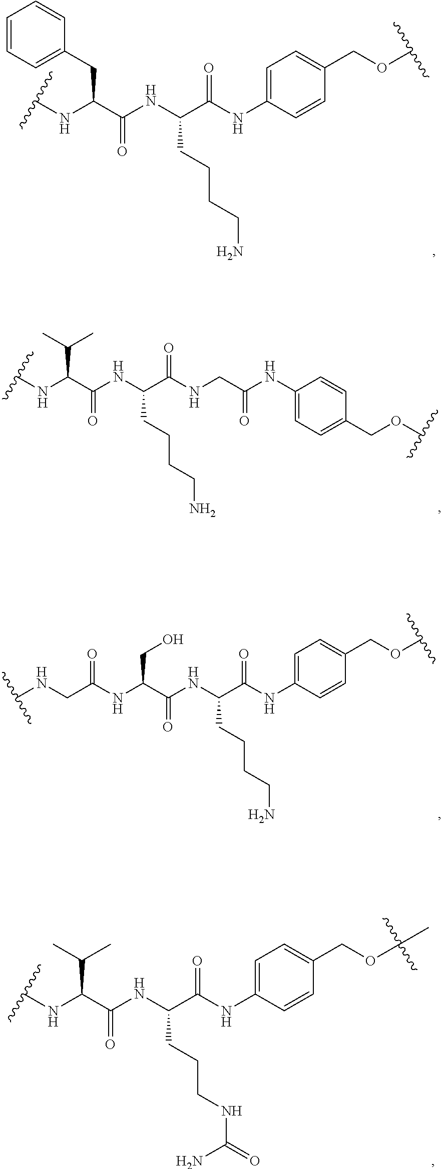 [SMILES: *N[C@@H](Cc1ccccc1)C(=O)N[C@@H](CCCCN)C(=O)Nc1ccc(CO*)cc1 ; *N[C@H](C(=O)N[C@@H](CCCCN)C(=O)NCC(=O)Nc1ccc(CO*)cc1)C(C)C ; *NCC(=O)N[C@@H](CO)C(=O)N[C@@H](CCCCN)C(=O)Nc1ccc(CO*)cc1 ; *N[C@H](C(=O)N[C@@H](CCCNC(N)=O)C(=O)Nc1ccc(CO*)cc1)C(C)C]

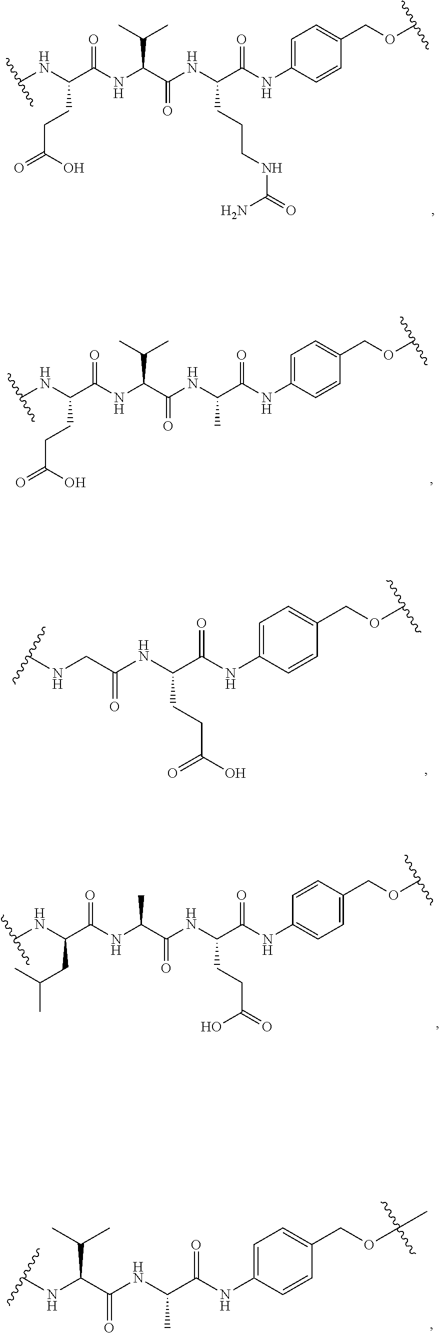 [SMILES: *N[C@@H](CCC(=O)O)C(=O)N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](C)C(=O)Nc1ccc(CO*)cc1)C(C)C ; *NCC(=O)N[C@@H](CCC(=O)O)C(=O)Nc1ccc(CO*)cc1 ; *N[C@H](CC(C)C)C(=O)N[C@@H](C)C(=O)N[C@@H](CCC(=O)O)C(=O)Nc1ccc(CO*)cc1 ; *N[C@H](C(=O)N[C@@H](C)C(=O)Nc1ccc(CO*)cc1)C(C)C]

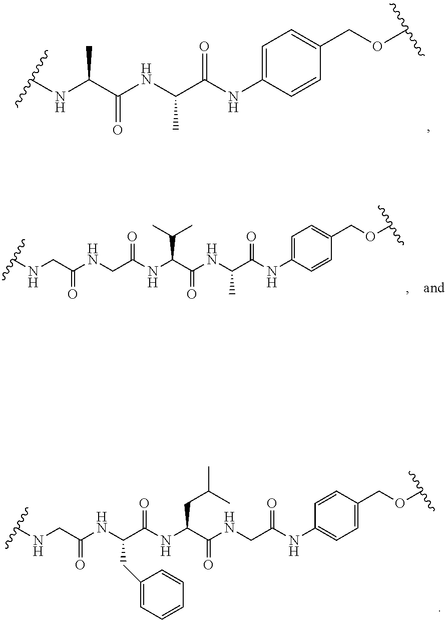 [SMILES: *N[C@@H](C)C(=O)N[C@@H](C)C(=O)Nc1ccc(CO*)cc1 ; *NCC(=O)NCC(=O)N[C@H](C(=O)N[C@@H](C)C(=O)Nc1ccc(CO*)cc1)C(C)C ; *NCC(=O)N[C@@H](Cc1ccccc1)C(=O)N[C@@H](CC(C)C)C(=O)NCC(=O)Nc1ccc(CO*)cc1]

In some embodiments, L comprises a structural fragment selected from the following:

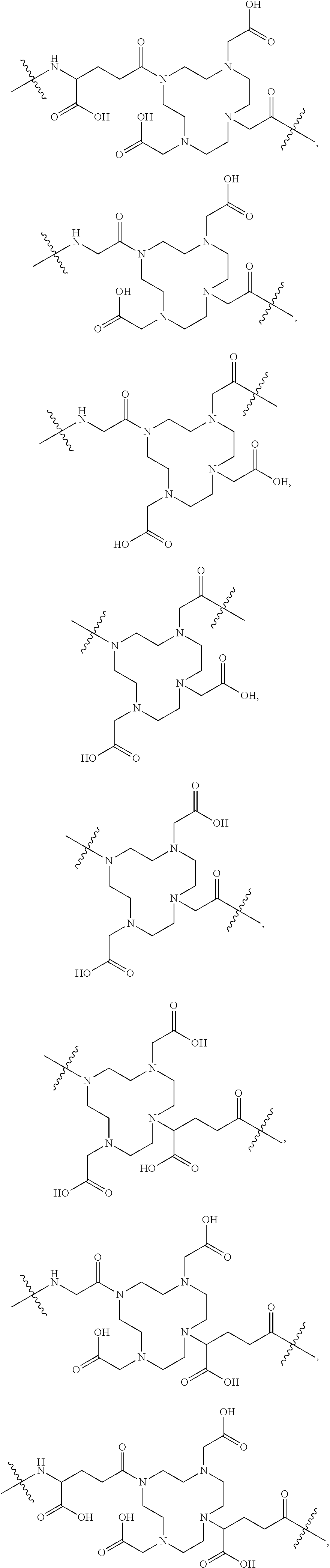

[chemical structure]

[chemical structure]

In some embodiments, L comprises a structural fragment selected from the following:

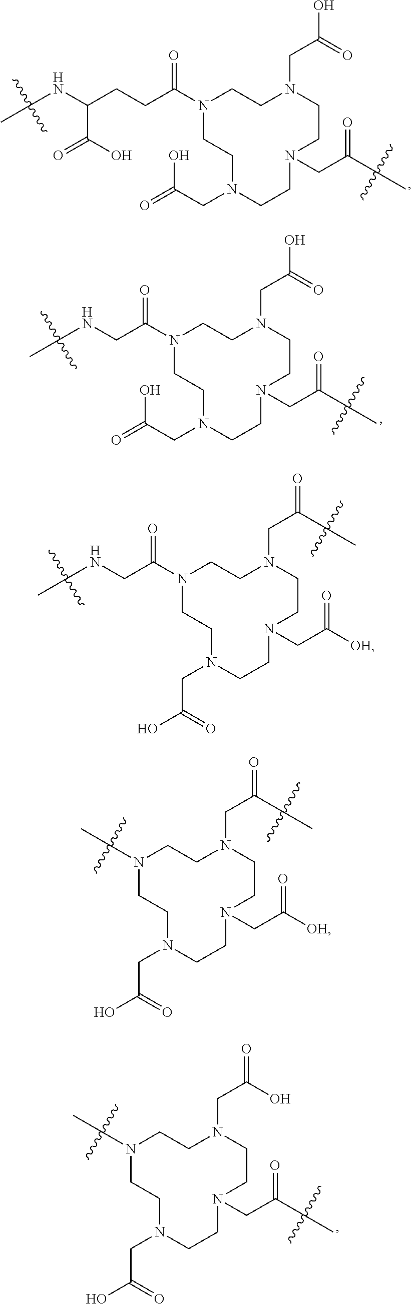

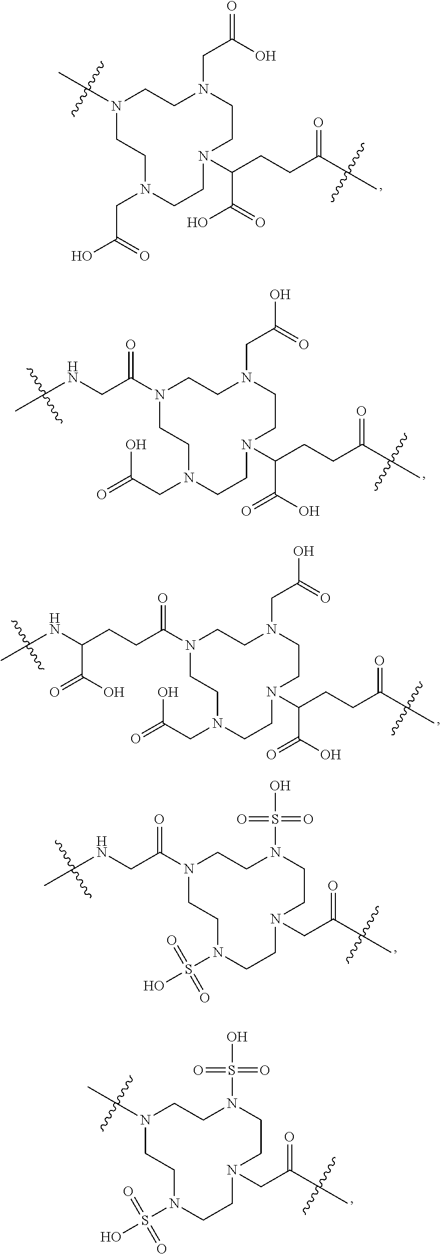

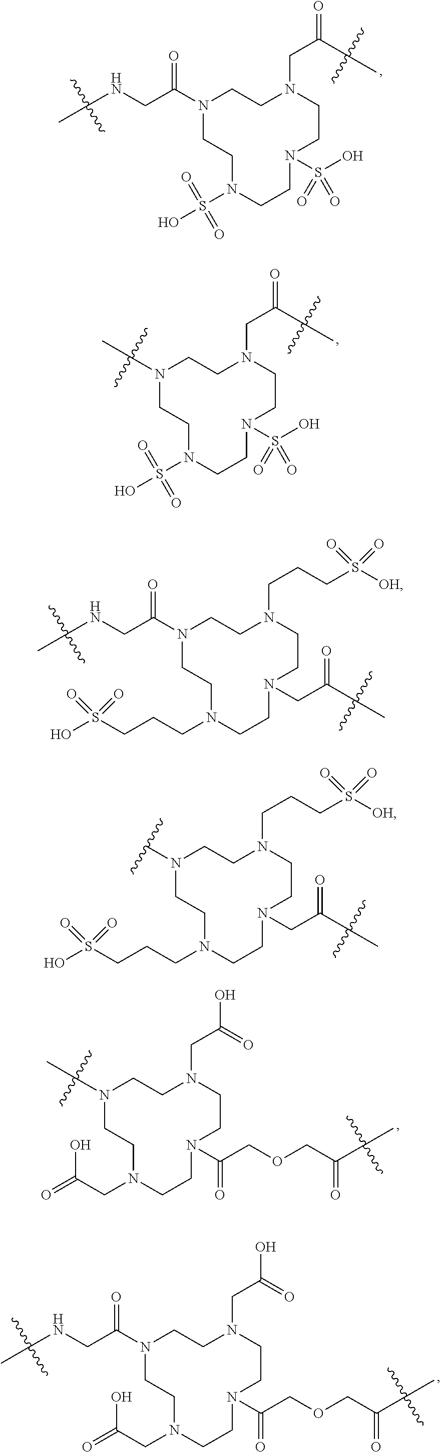

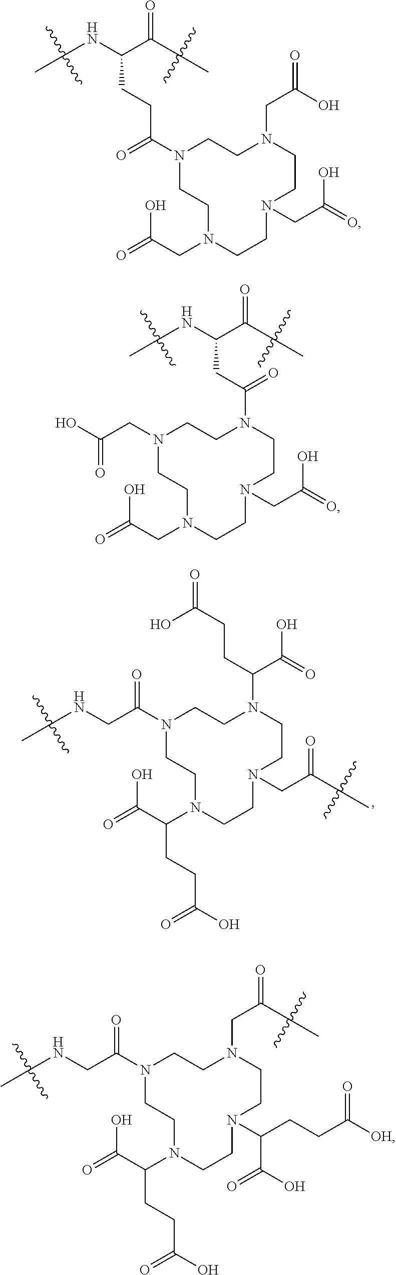

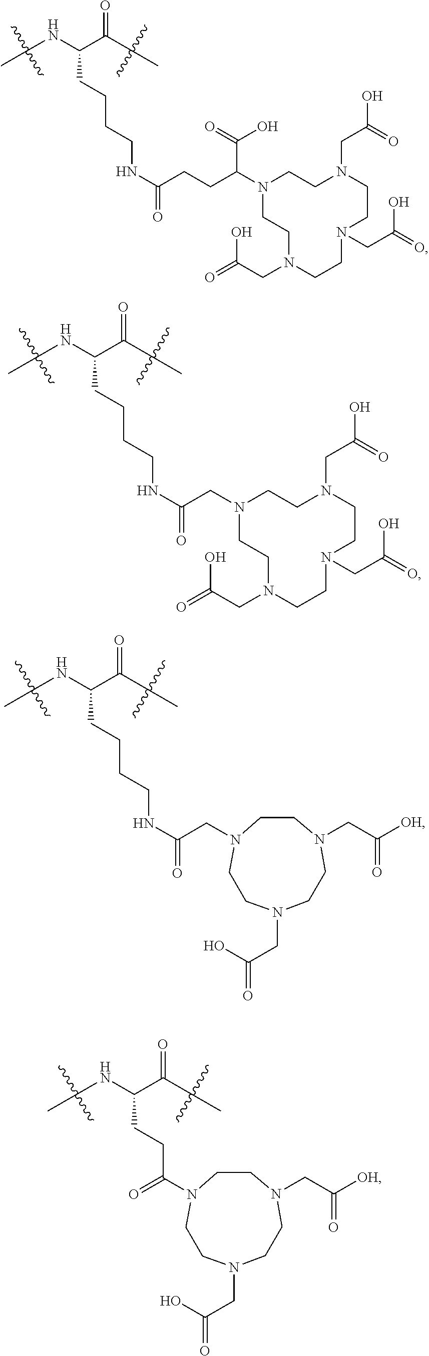 [SMILES: *N[C@@H](CCCCNC(=O)CCC(C(=O)O)N1CCN(CC(=O)O)CCN(CC(=O)O)CCN(CC(=O)O)CC1)C(*)=O ; *N[C@@H](CCCCNC(=O)CN1CCN(CC(=O)O)CCN(CC(=O)O)CCN(CC(=O)O)CC1)C(*)=O ; *N[C@@H](CCCCNC(=O)CN1CCN(CC(=O)O)CCN(CC(=O)O)CC1)C(*)=O ; *N[C@@H](CCC(=O)N1CCN(CC(=O)O)CCN(CC(=O)O)CC1)C(*)=O]

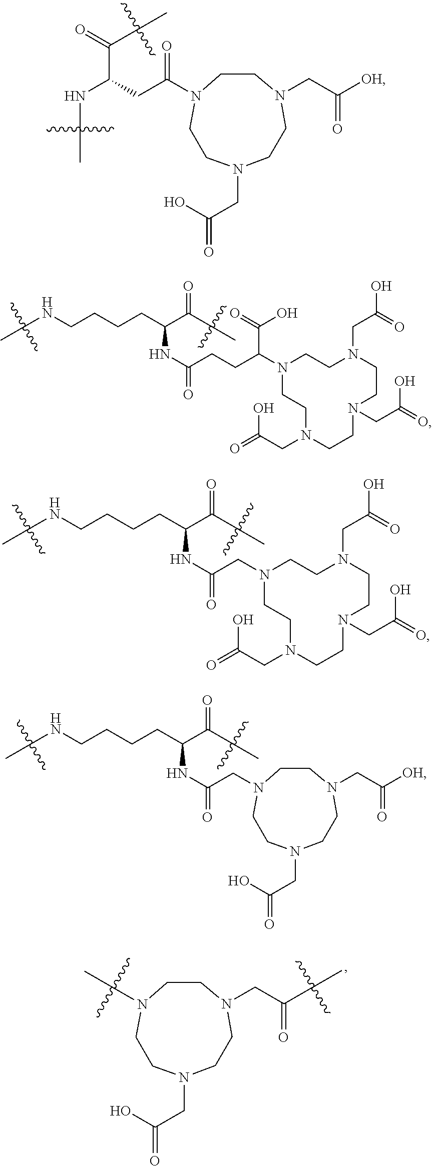

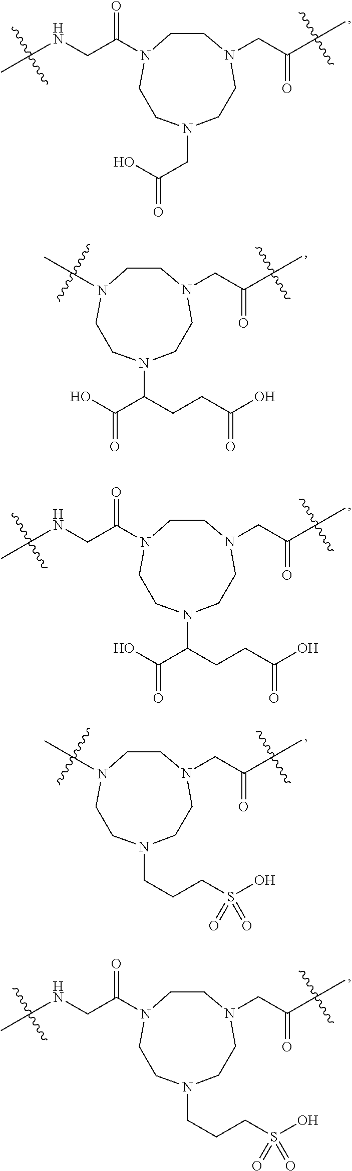

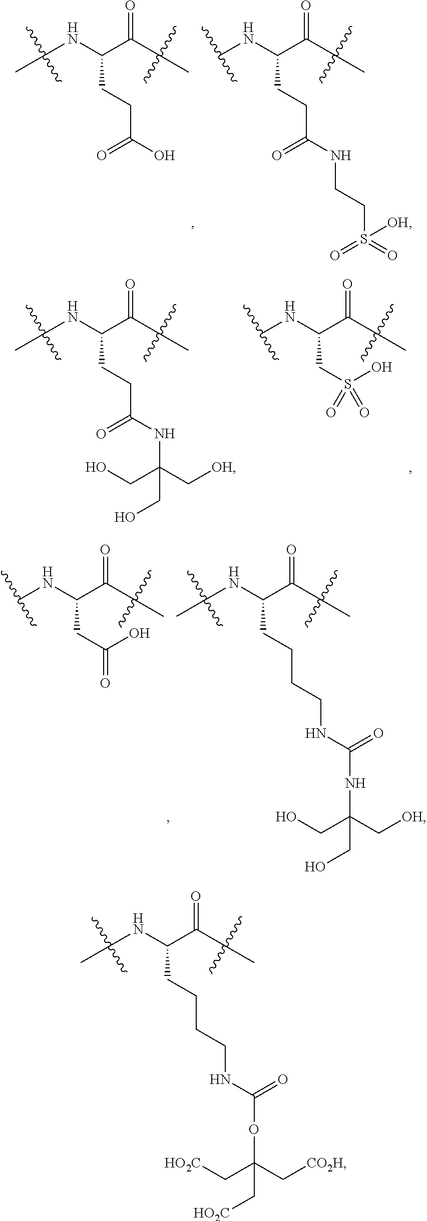

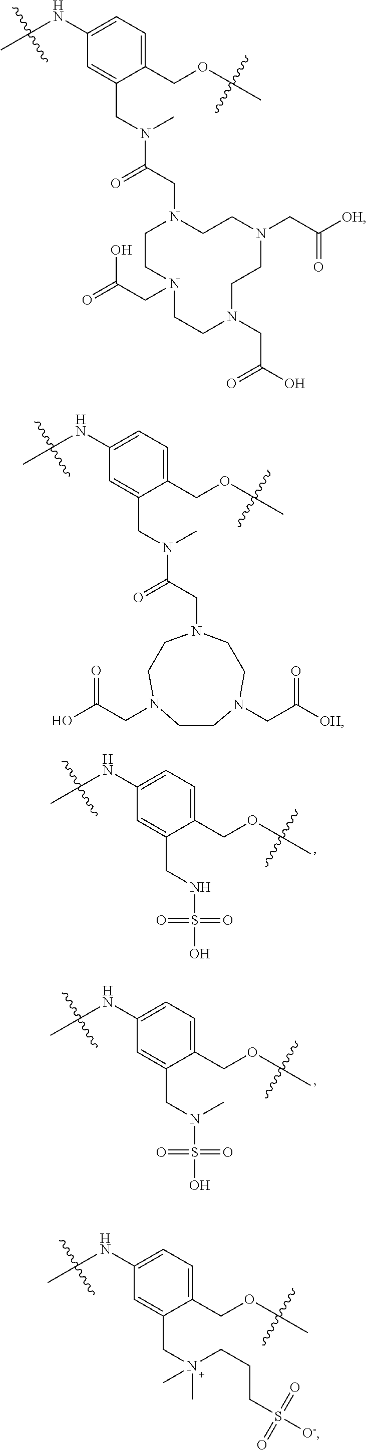

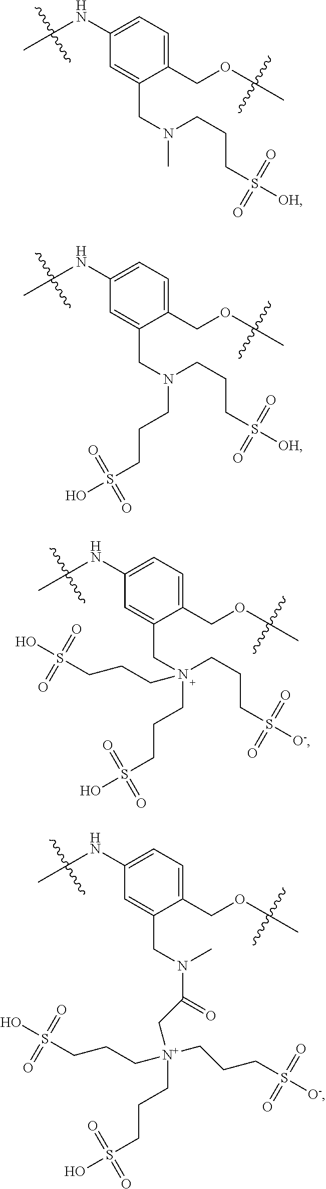

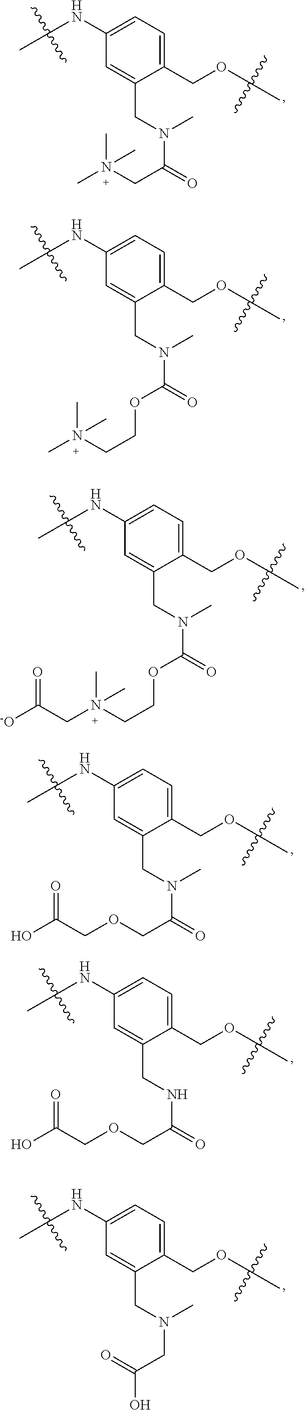

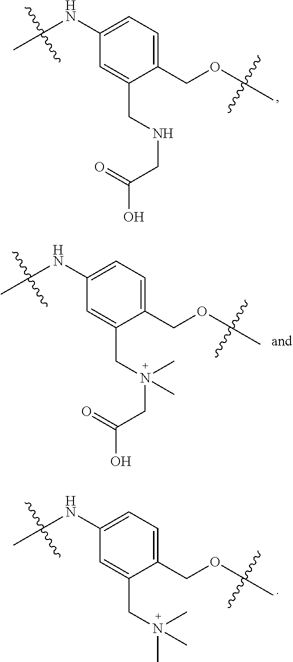

In some embodiments, L is selected from substituted or unsubstituted structural fragments composed of one or more of the following groups:

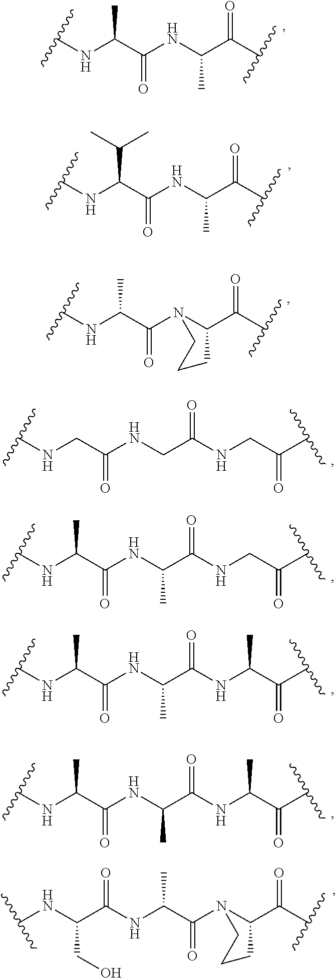

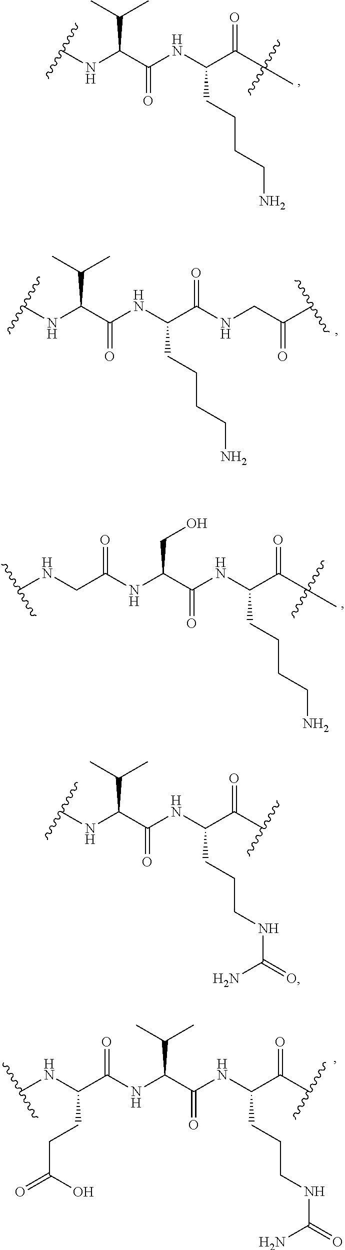 [SMILES: *N[C@H](C(=O)N[C@@H](CCCCN)C(*)=O)C(C)C ; *N[C@H](C(=O)N[C@@H](CCCCN)C(=O)NCC(*)=O)C(C)C ; *NCC(=O)N[C@@H](CO)C(=O)N[C@@H](CCCCN)C(*)=O ; *N[C@H](C(=O)N[C@@H](CCCNC(N)=O)C(*)=O)C(C)C ; *N[C@@H](CCC(=O)O)C(=O)N[C@H](C(=O)N[C@@H](CCCNC(N)=O)C(*)=O)C(C)C]

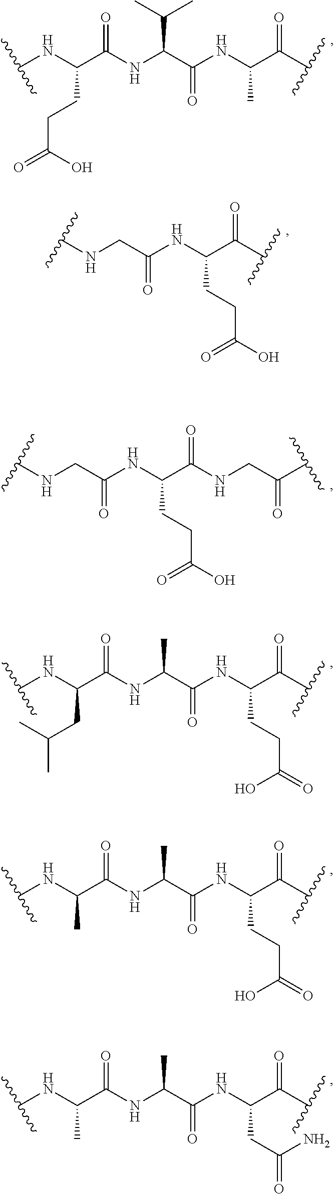 [SMILES: *N[C@@H](CCC(=O)O)C(=O)N[C@H](C(=O)N[C@@H](C)C(*)=O)C(C)C ; *NCC(=O)N[C@@H](CCC(=O)O)C(*)=O ; *NCC(=O)N[C@@H](CCC(=O)O)C(=O)NCC(*)=O ; *N[C@H](CC(C)C)C(=O)N[C@@H](C)C(=O)N[C@@H](CCC(=O)O)C(*)=O ; *N[C@H](C)C(=O)N[C@@H](C)C(=O)N[C@@H](CCC(=O)O)C(*)=O ; *N[C@@H](C)C(=O)N[C@@H](C)C(=O)N[C@@H](CC(N)=O)C(*)=O]

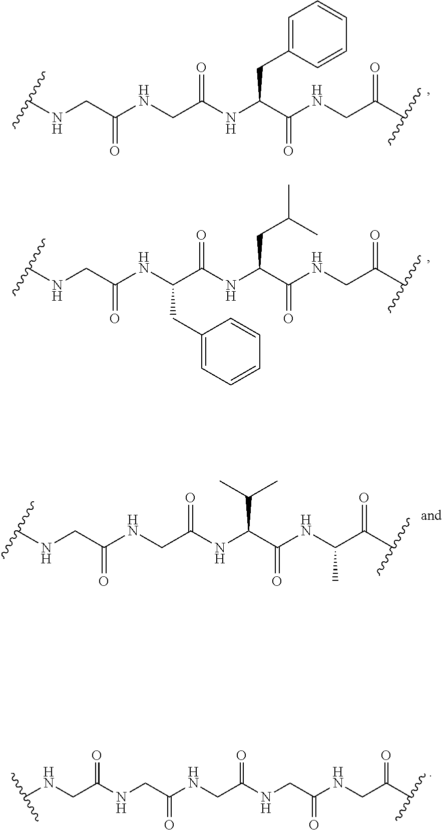

In some embodiments, L is selected from the group consisting of the following substituted or unsubstituted structural fragments:

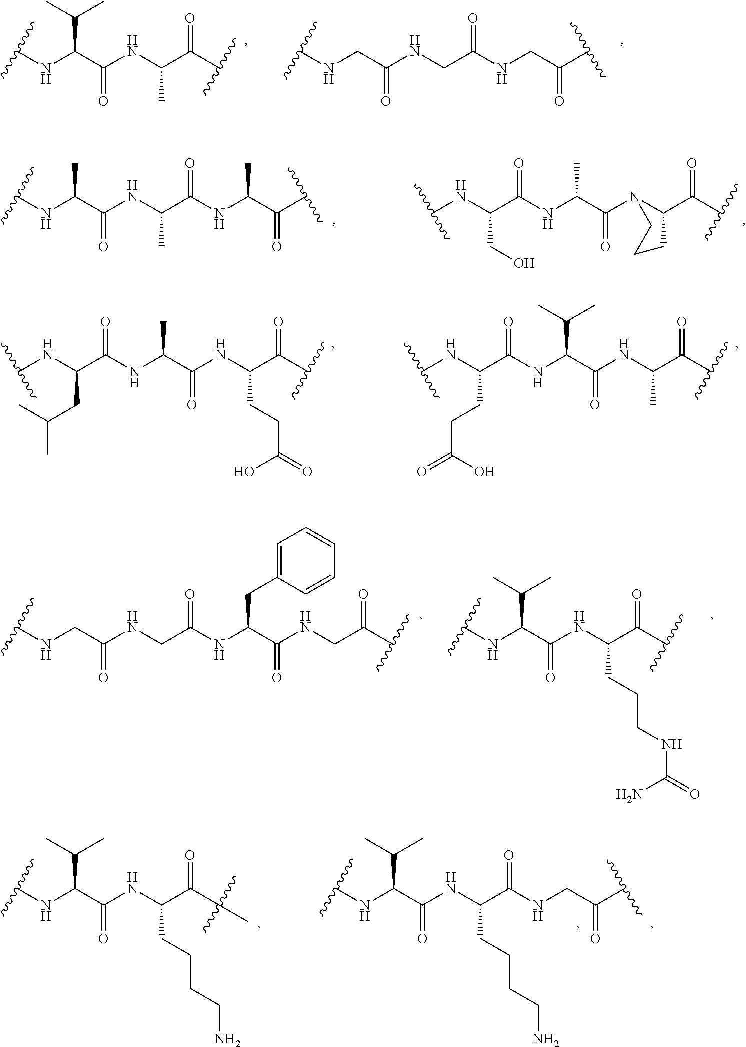

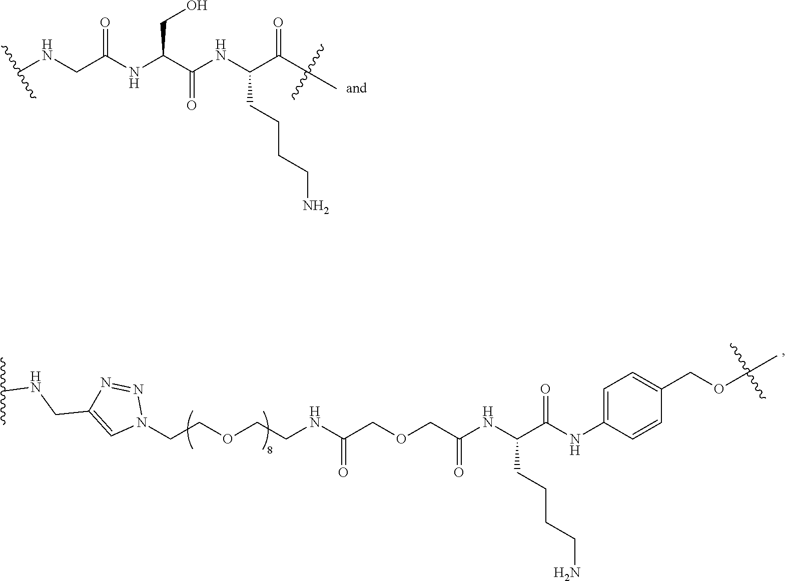

In some embodiments, L is selected from the group consisting of substituted or unsubstituted structural fragments composed of one or more of the following groups:

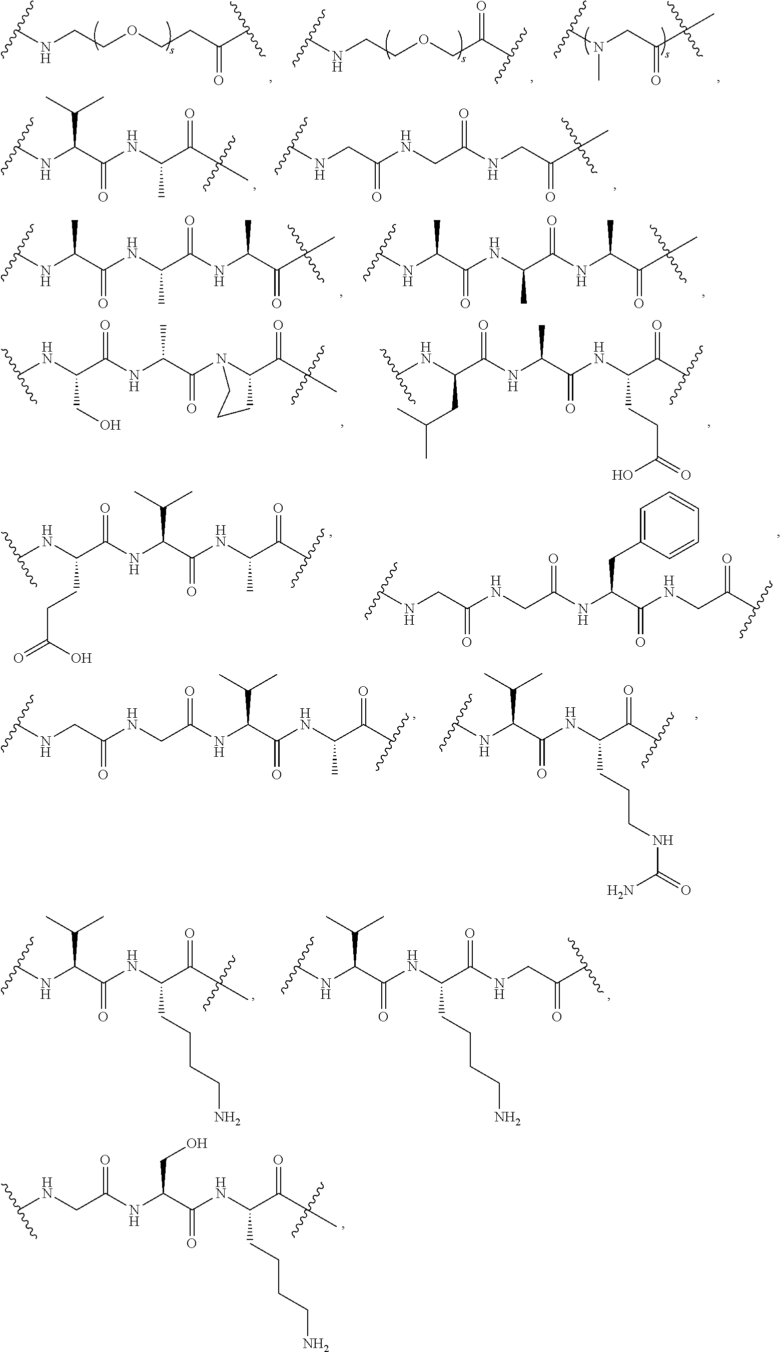

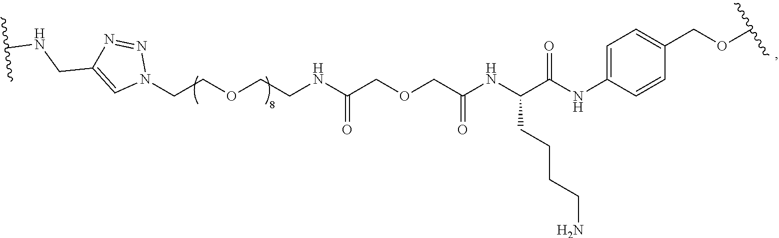

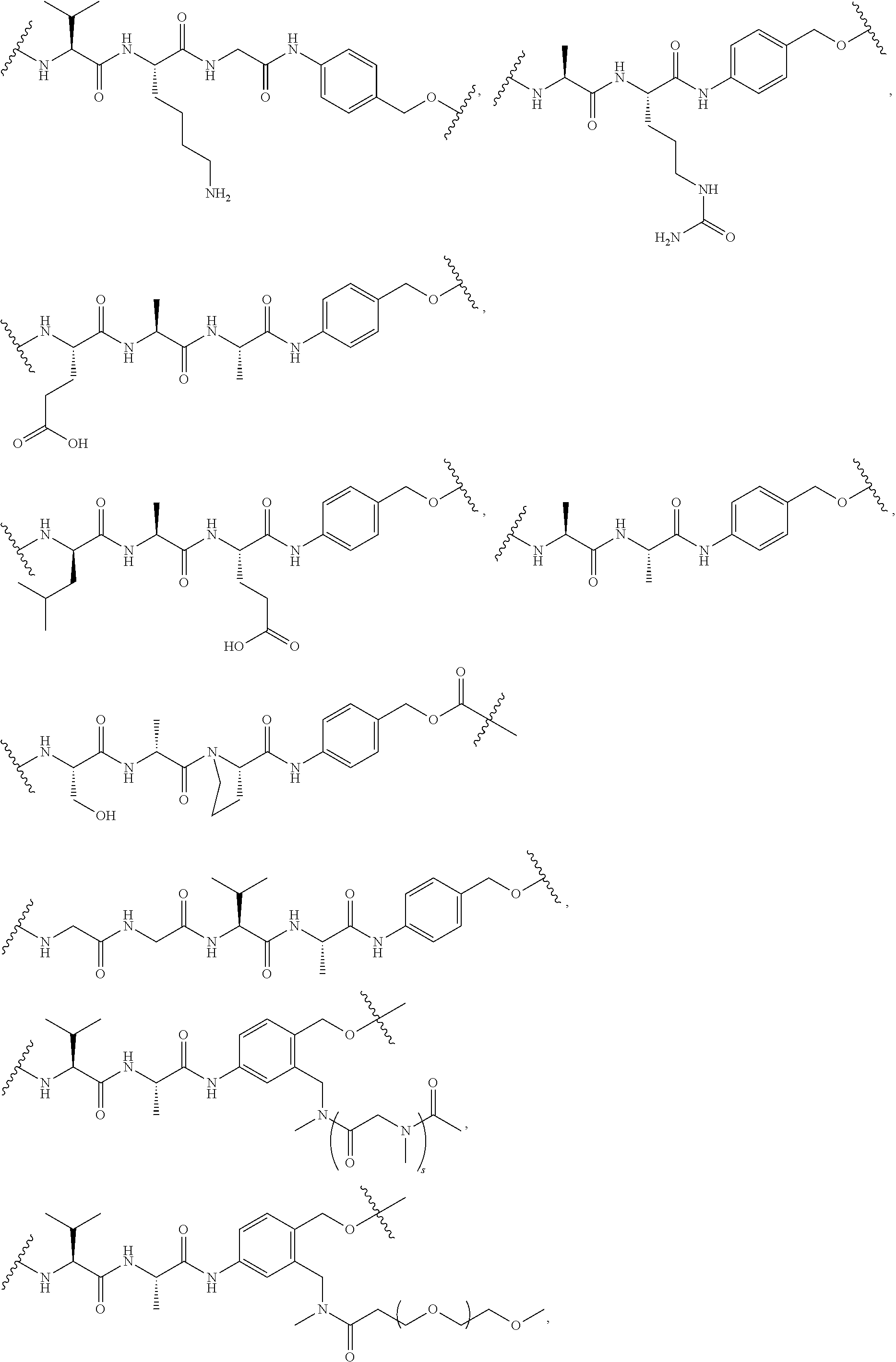

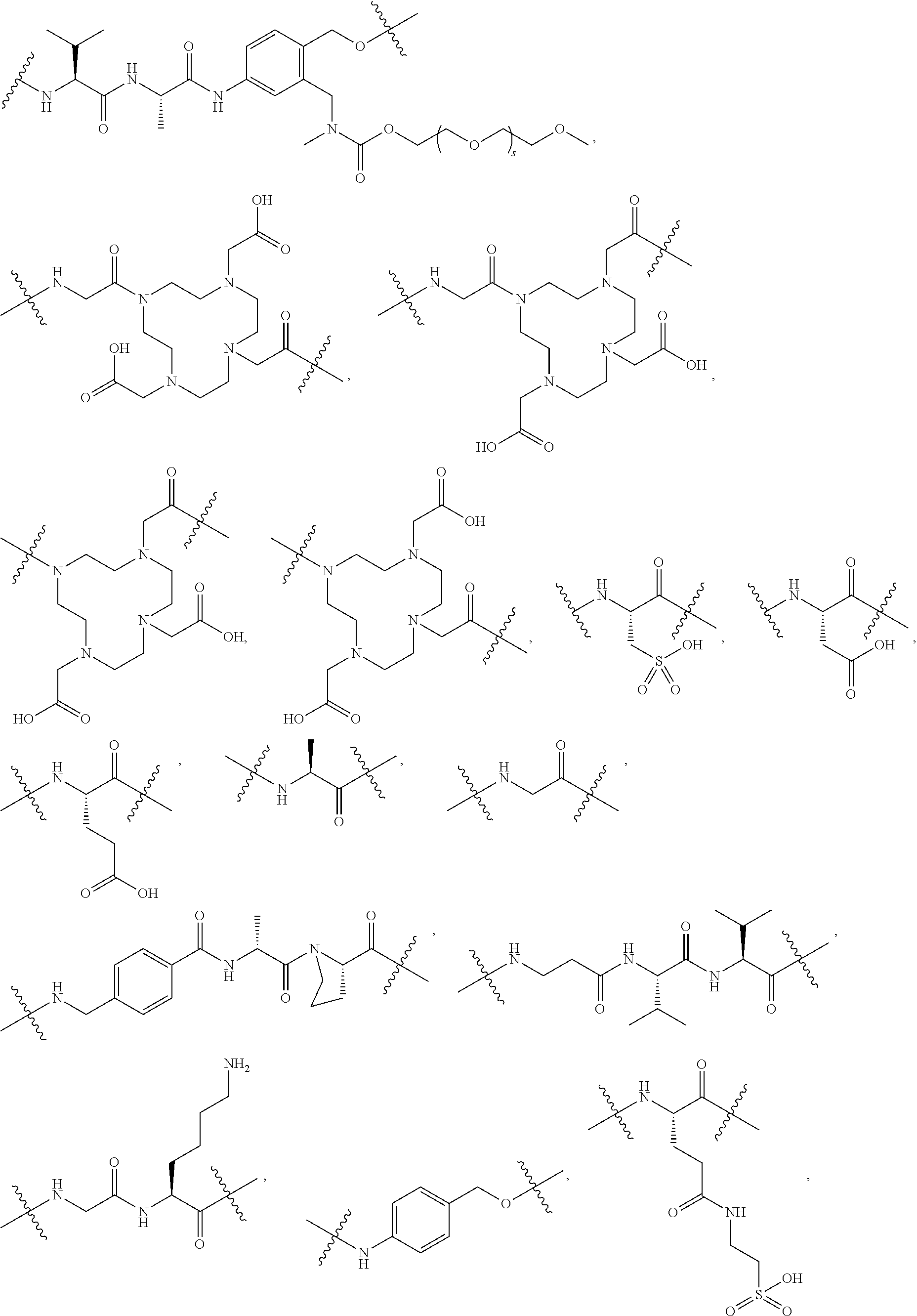

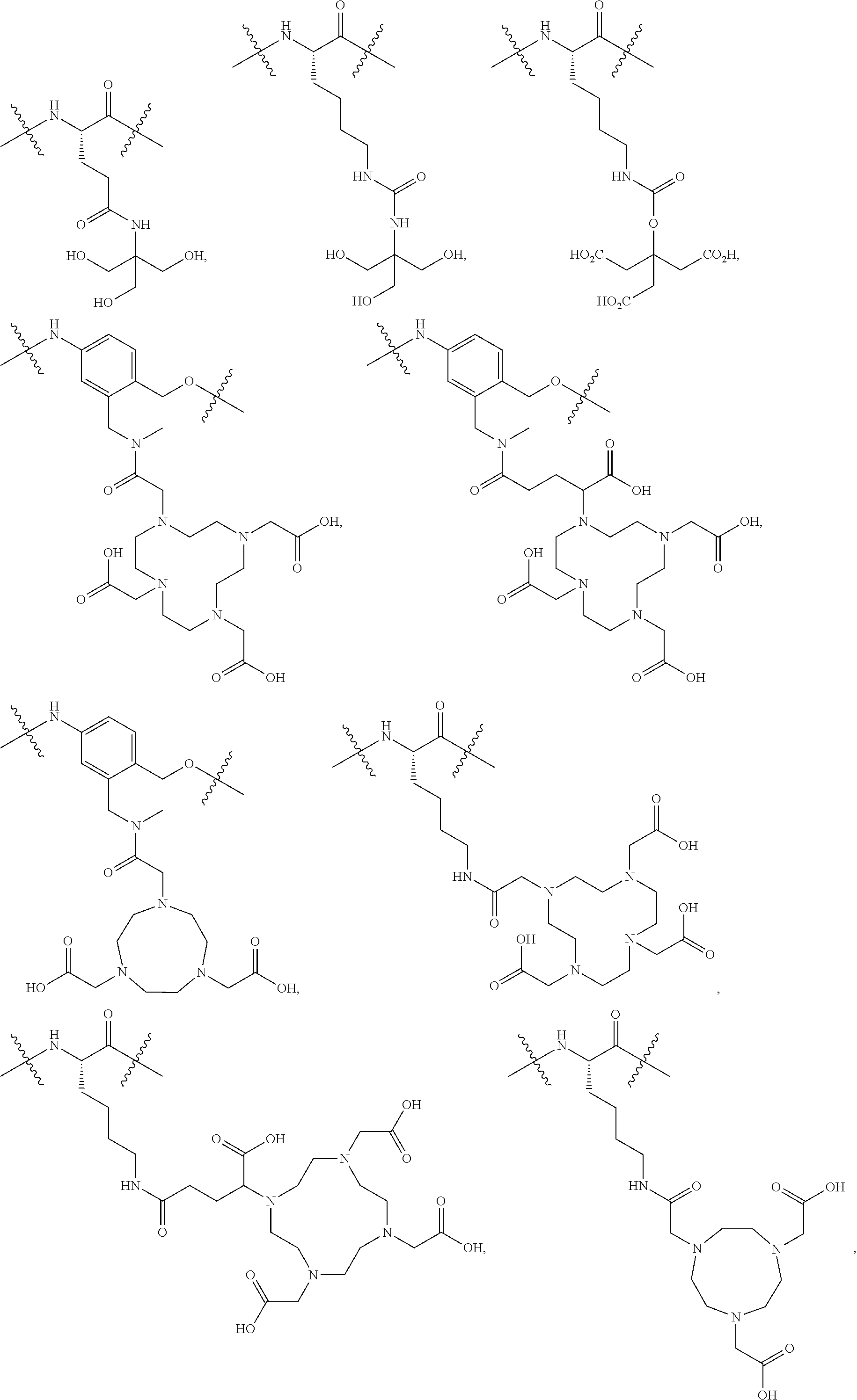

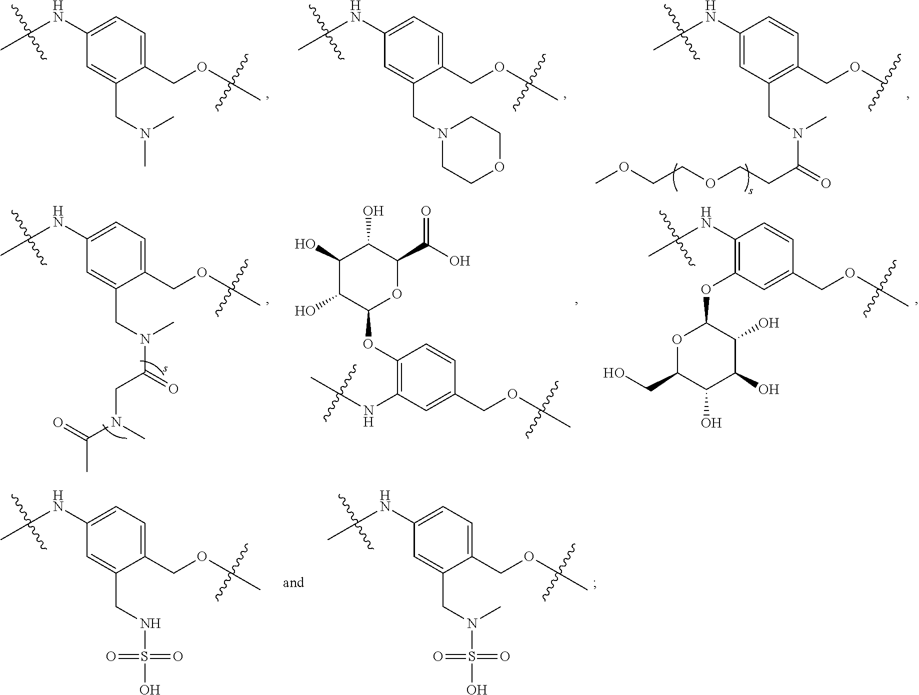

s is an integer selected from 1 to 20.

In some embodiments, L is selected from the group consisting of substituted or unsubstituted structural fragments composed of one or more of the following groups:

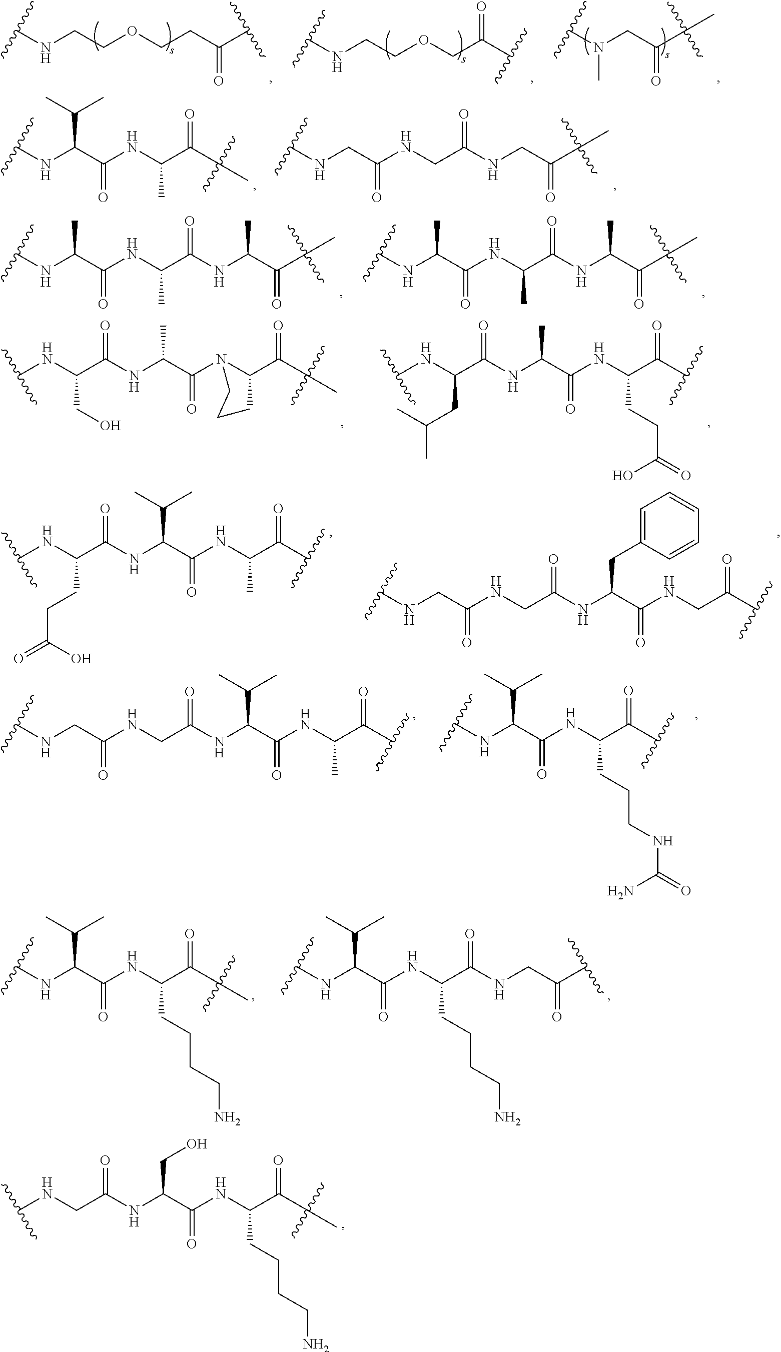

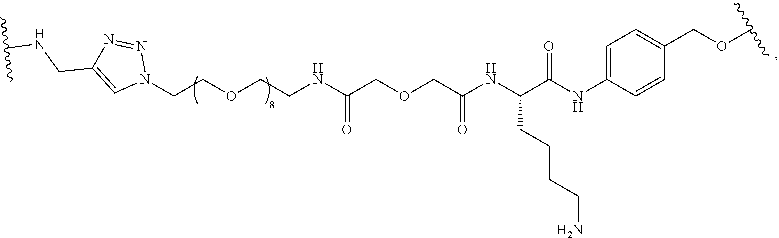

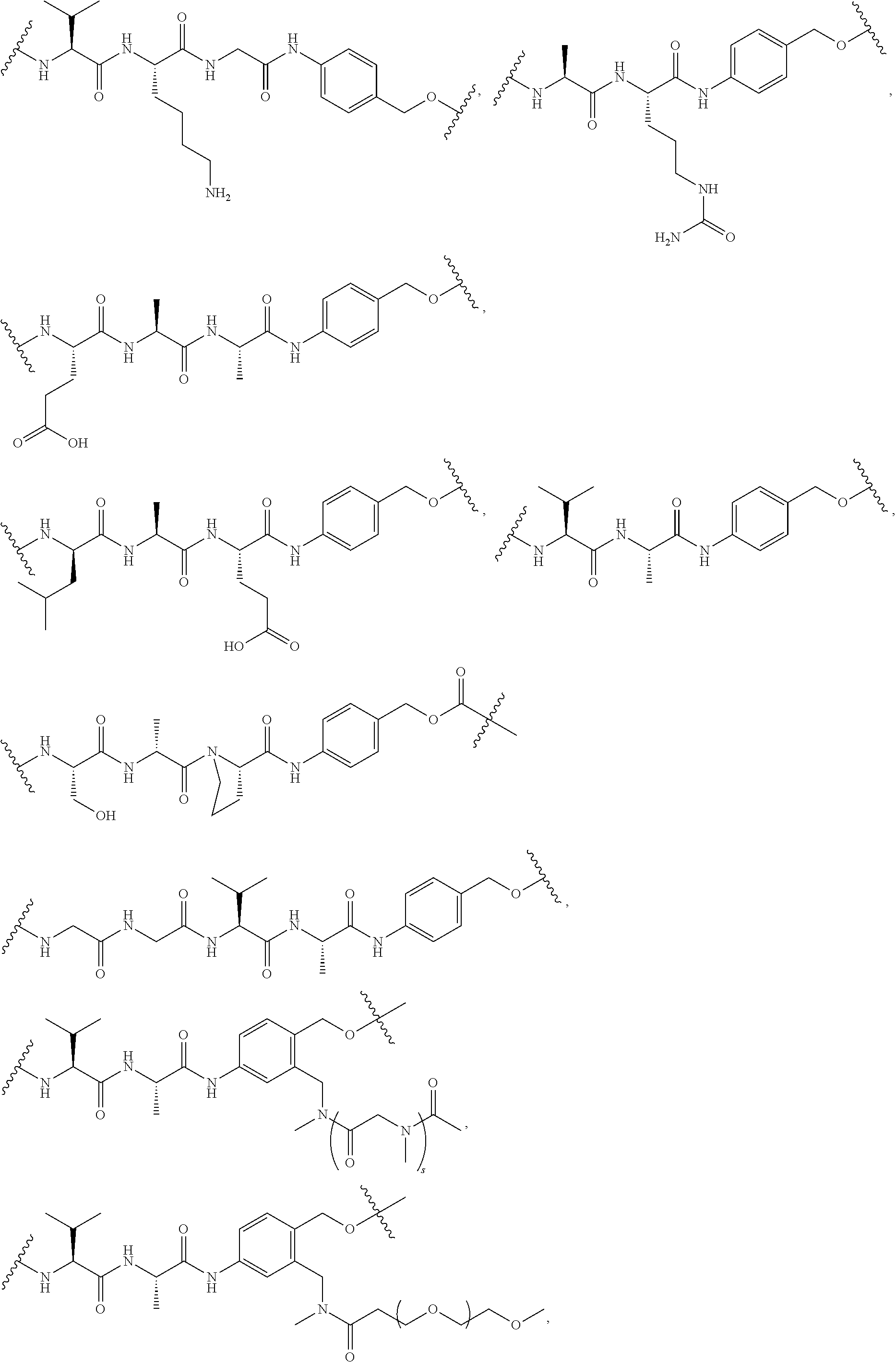

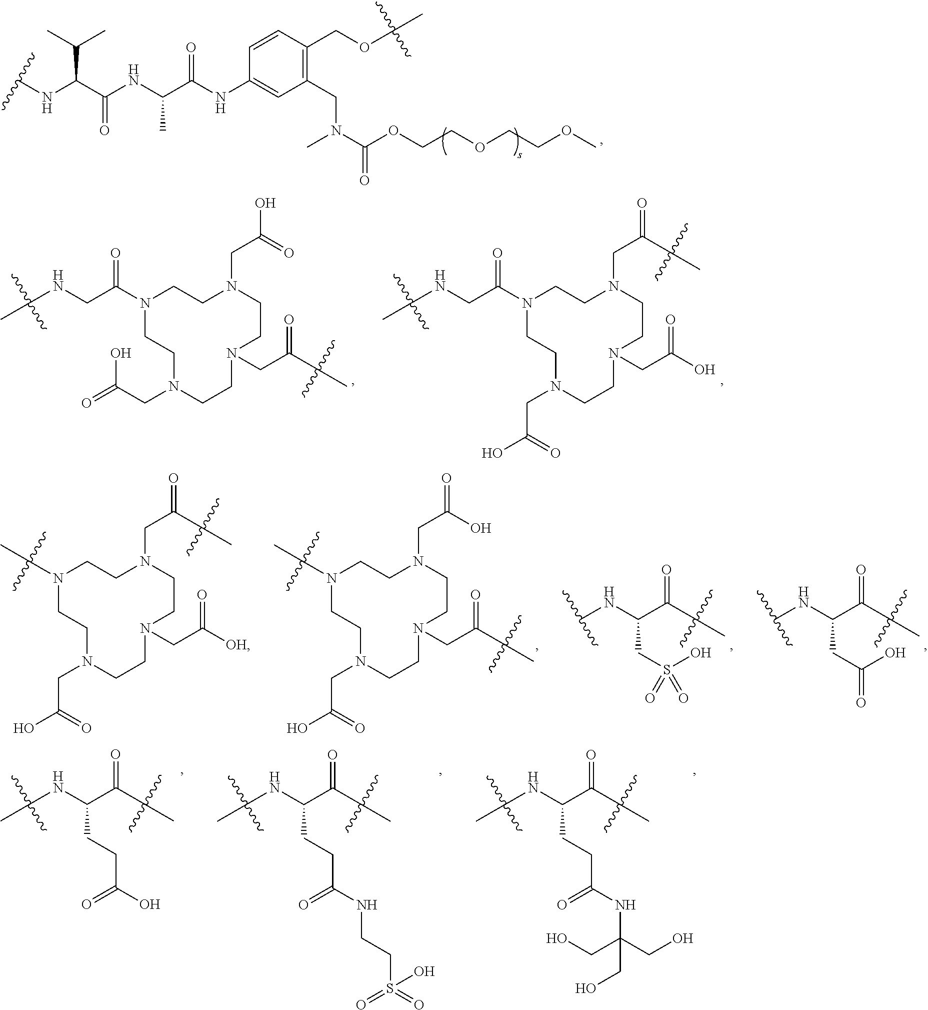

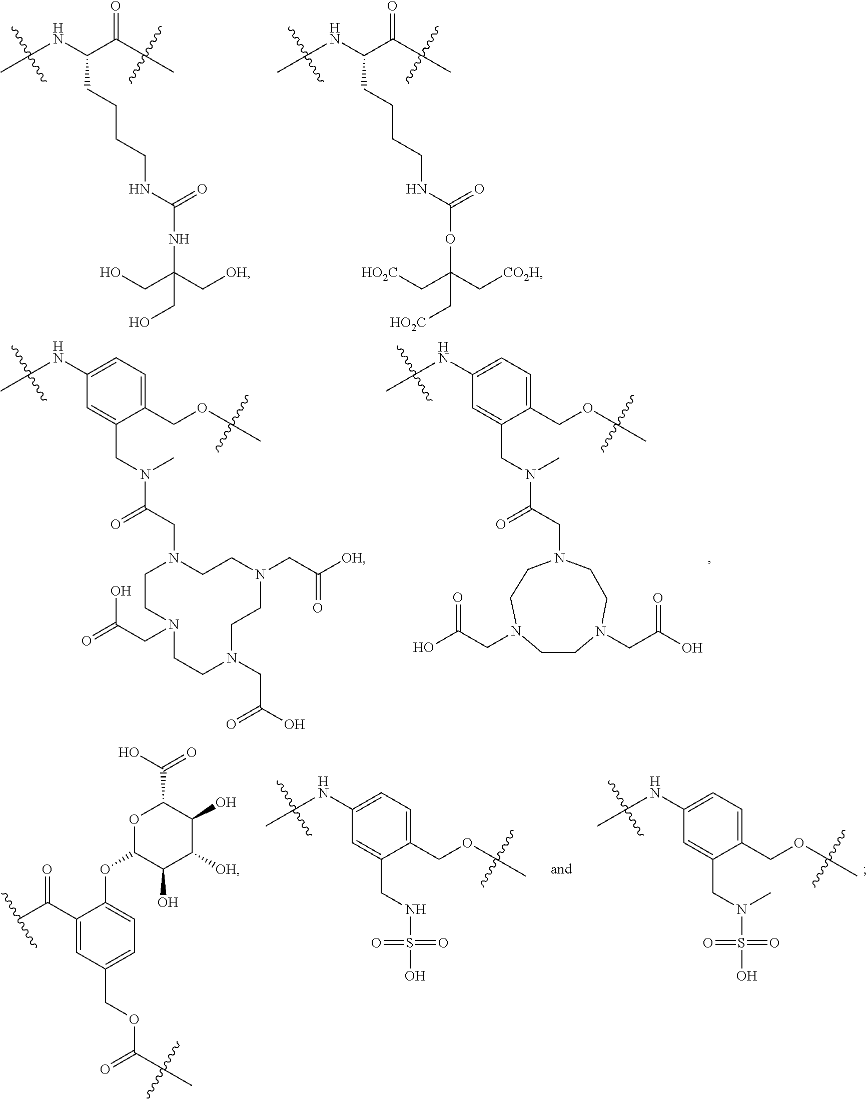

s is an integer selected from 1 to 20.

In some embodiments, L is selected from the group consisting of substituted or unsubstituted structural fragments composed of one or more of the following groups:

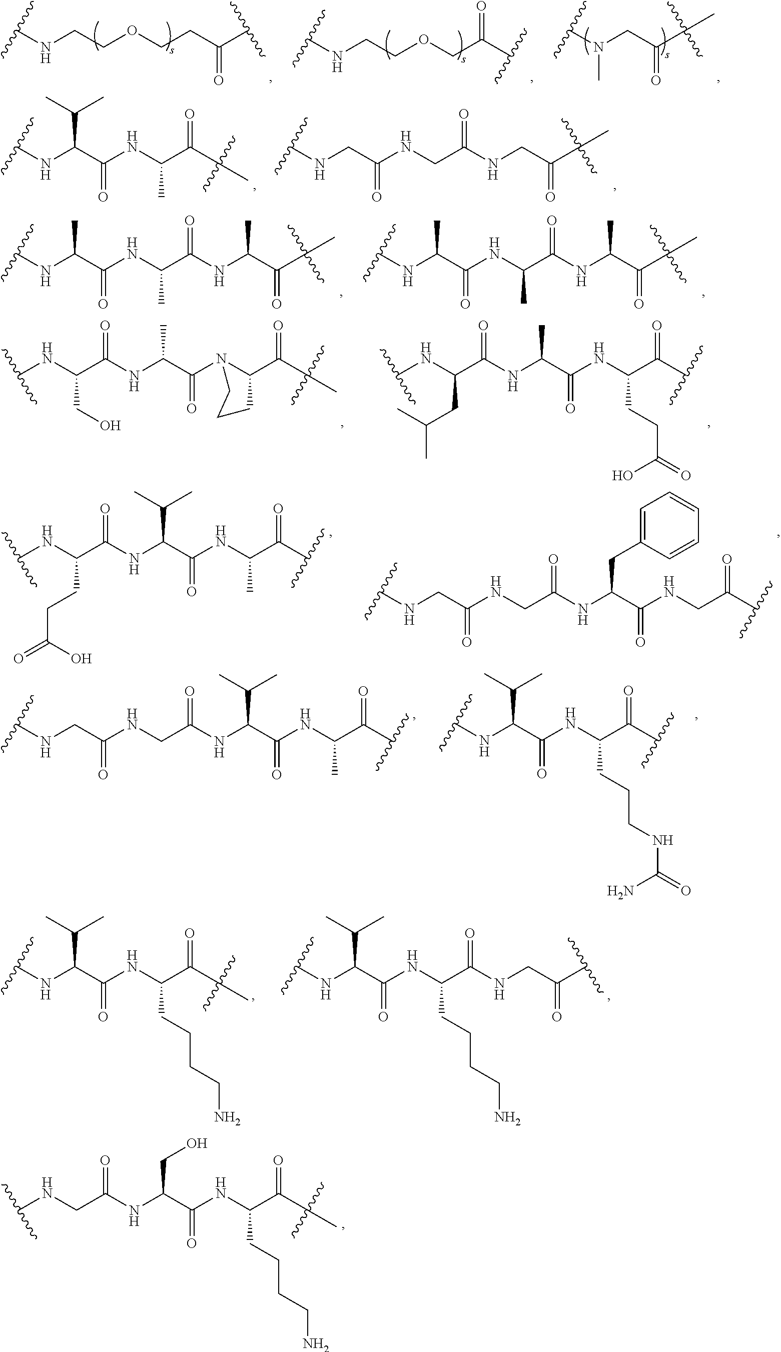

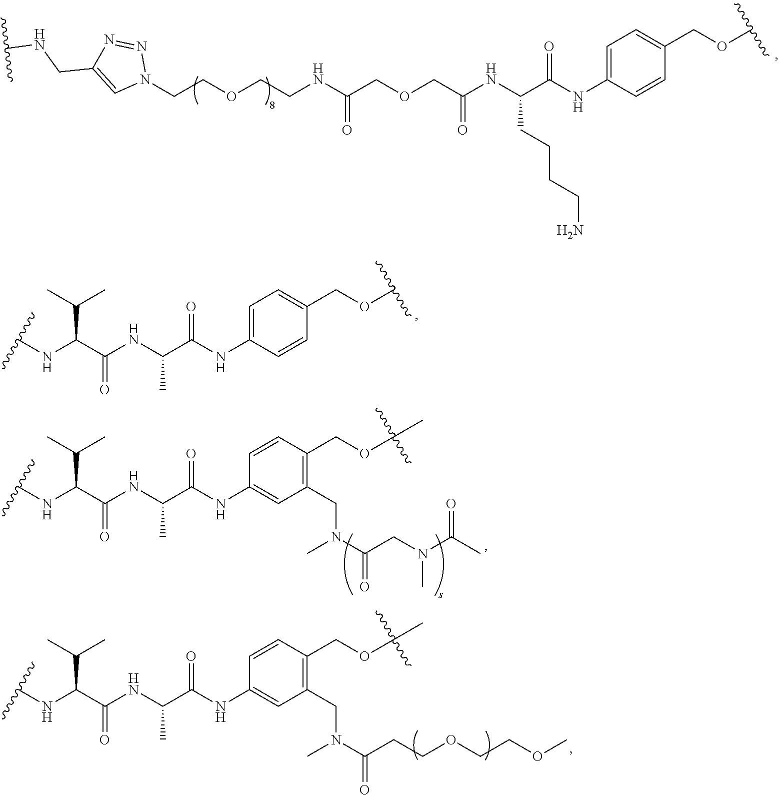

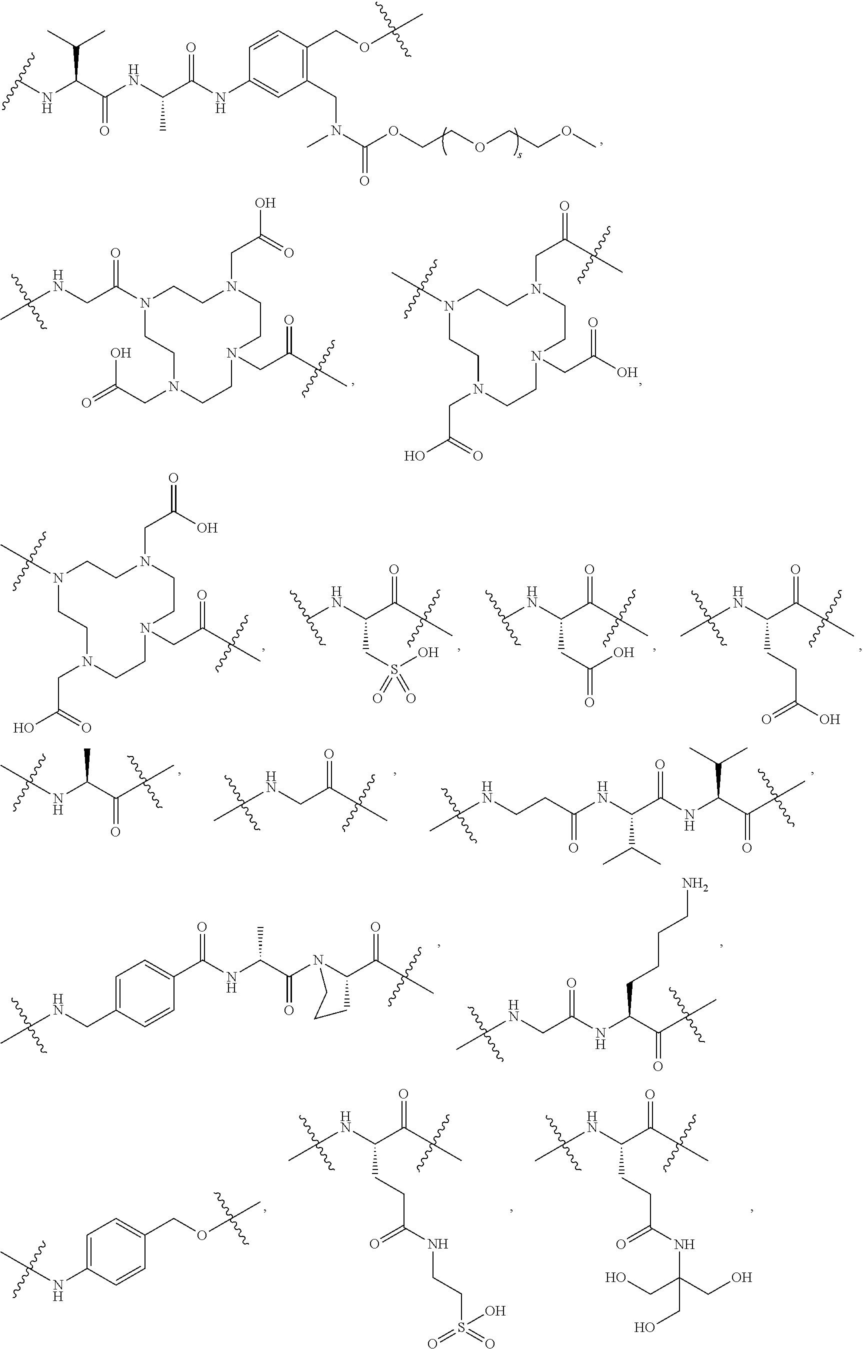

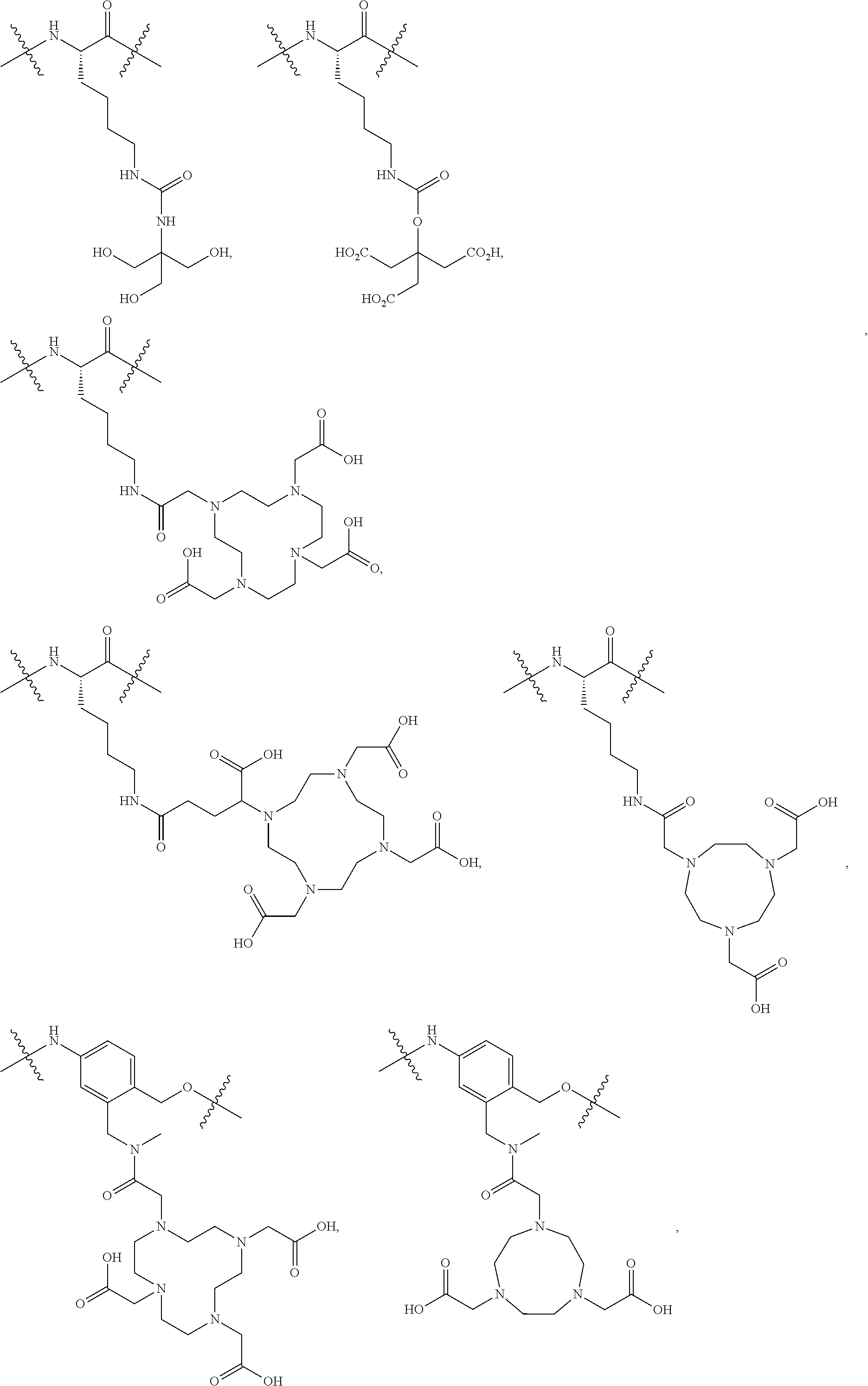

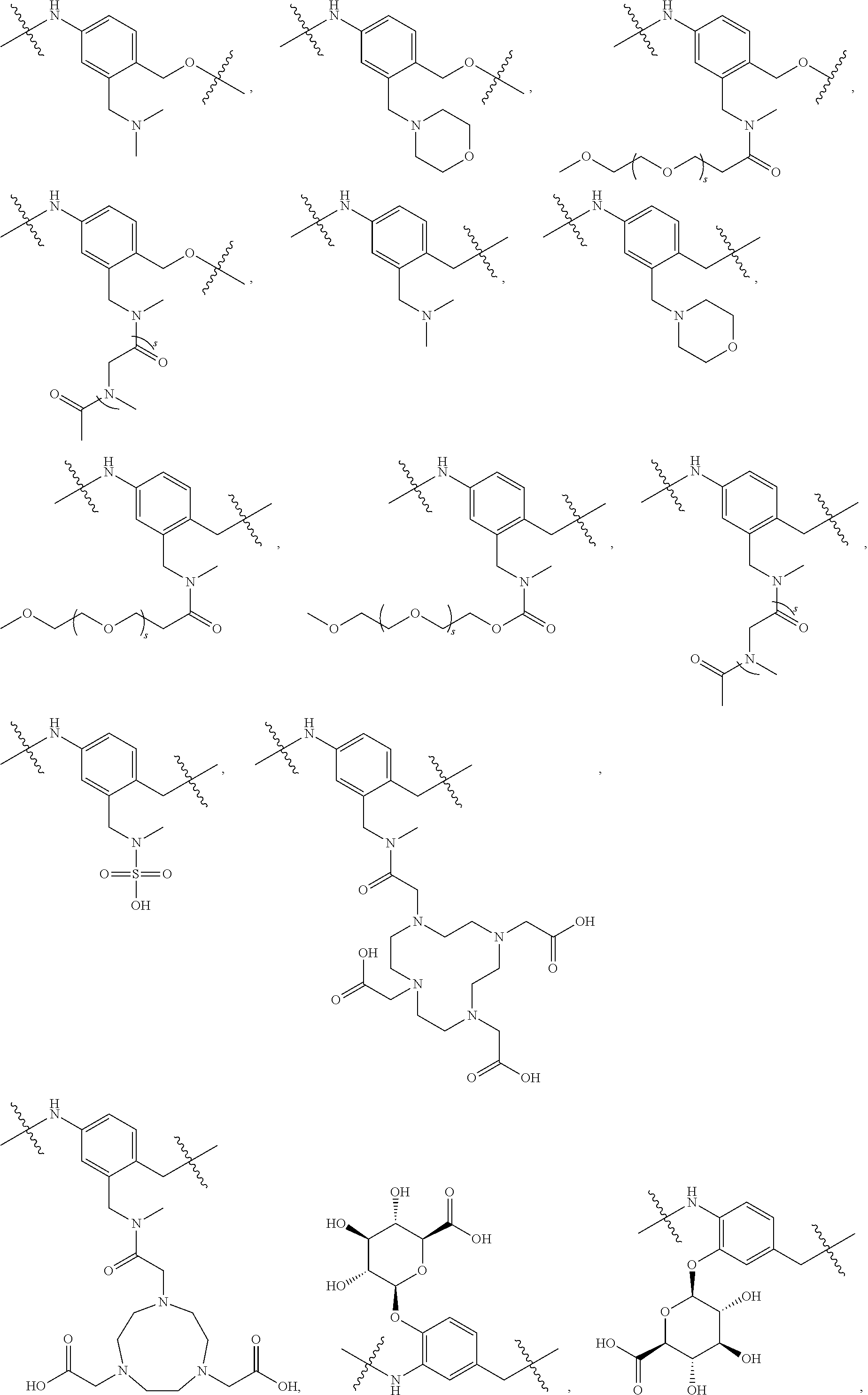

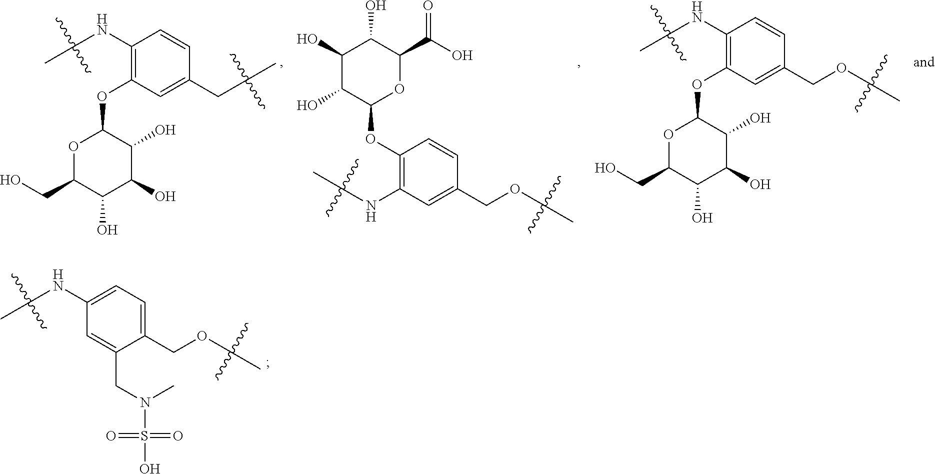 [SMILES: *Cc1ccc(N*)c(O[C@@H]2O[C@H](CO)[C@@H](O)[C@H](O)[C@H]2O)c1 ; *Nc1cc(CO*)ccc1O[C@@H]1O[C@H](C(=O)O)[C@@H](O)[C@H](O)[C@H]1O ; *Nc1ccc(CO*)cc1O[C@@H]1O[C@H](CO)[C@@H](O)[C@H](O)[C@H]1O ; *Nc1ccc(CO*)c(CN(C)S(=O)(=O)O)c1]

s is an integer Selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.

In some embodiments, L is selected from the group consisting of substituted or unsubstituted structural fragments composed of one or more of the following groups:

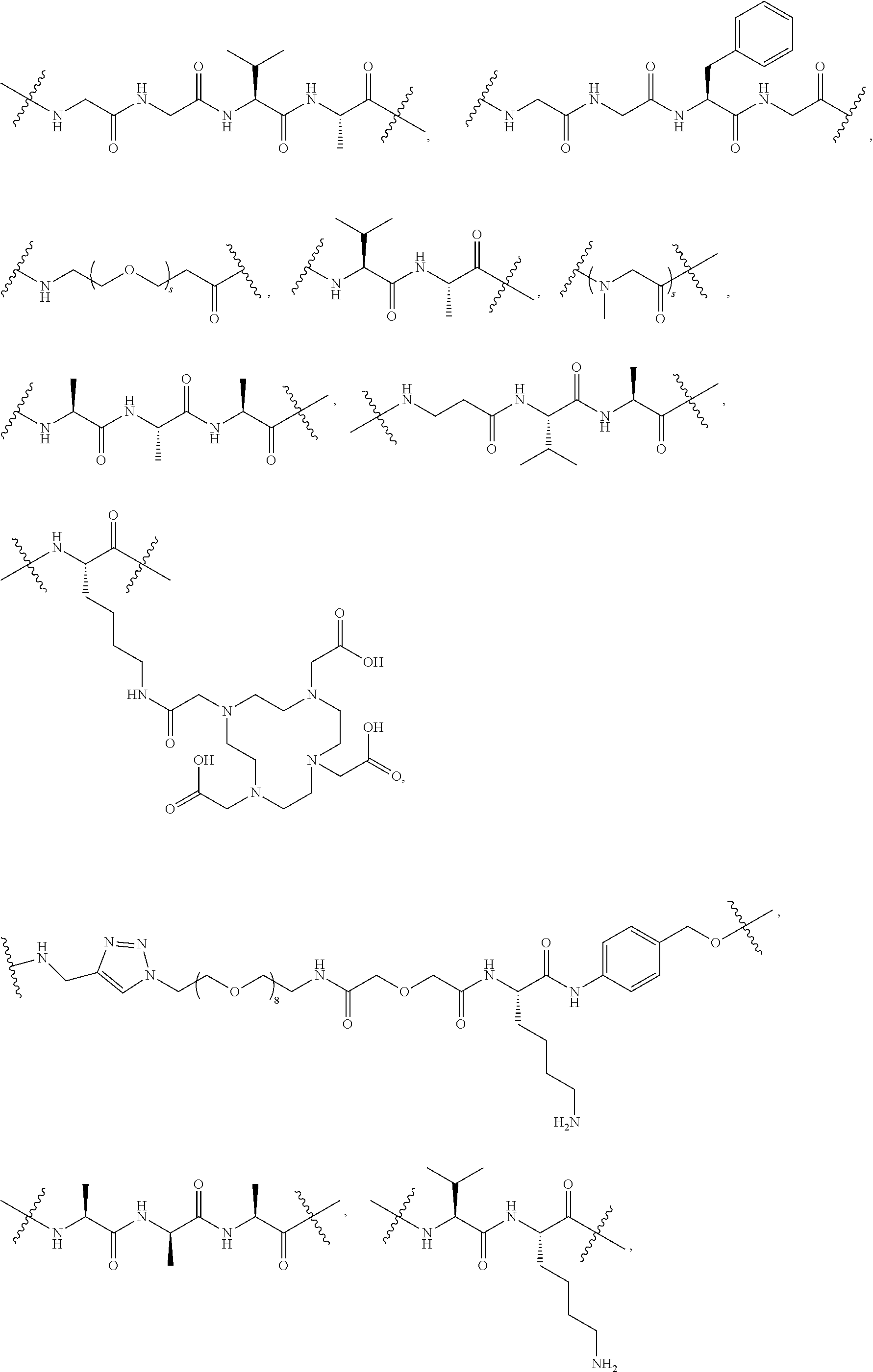

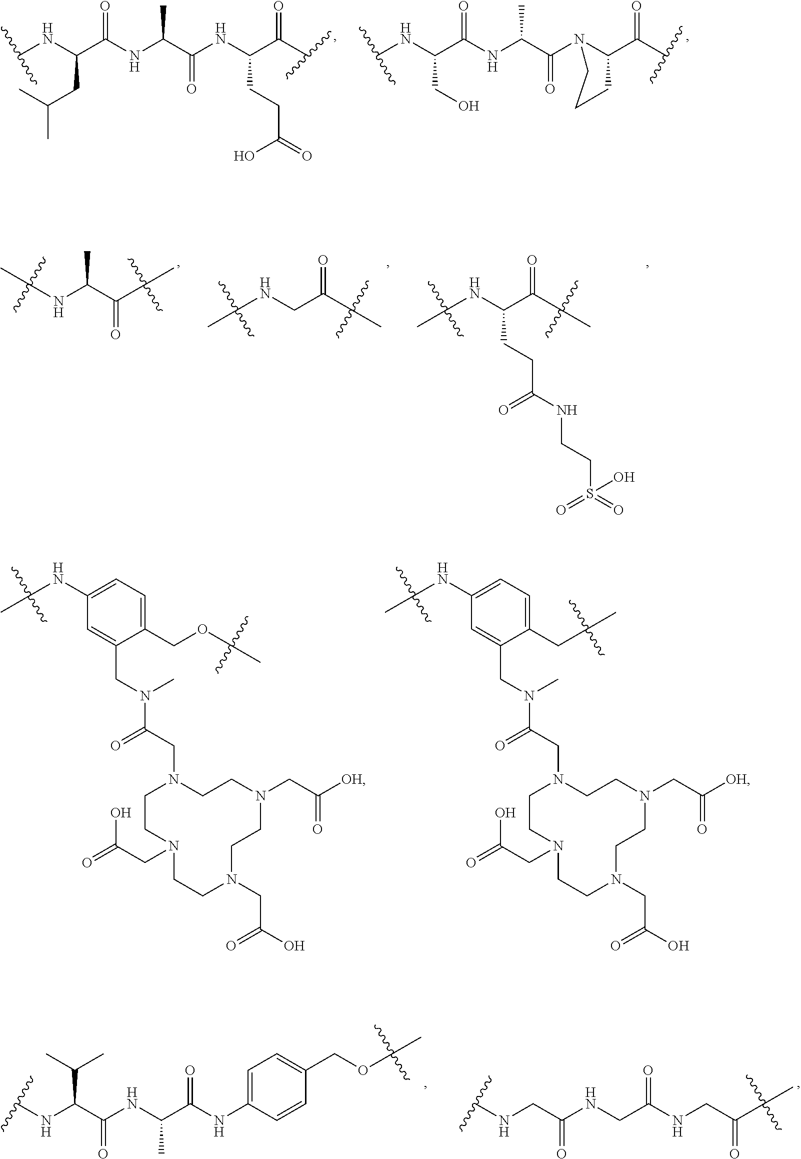

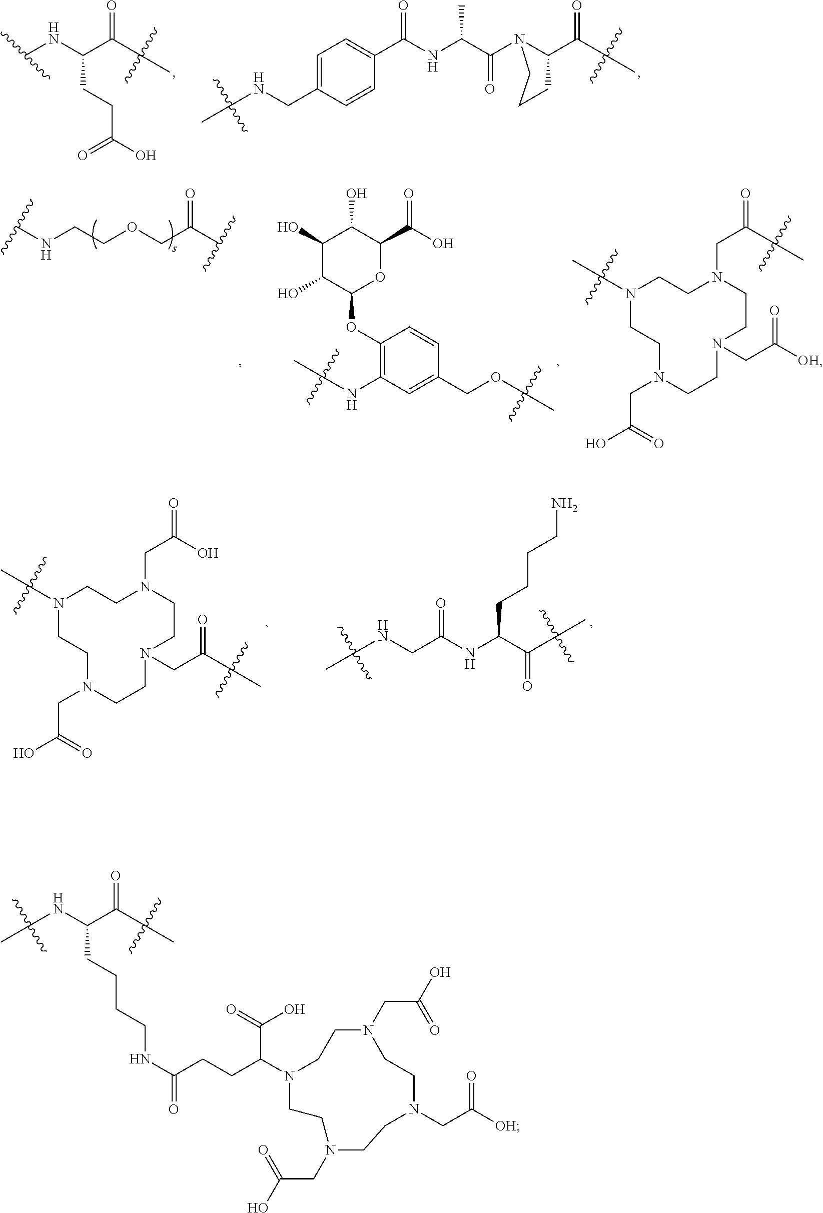

s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.

In some embodiments, L is selected from the group consisting of substituted or unsubstituted structural fragments composed of one or more of the following groups:

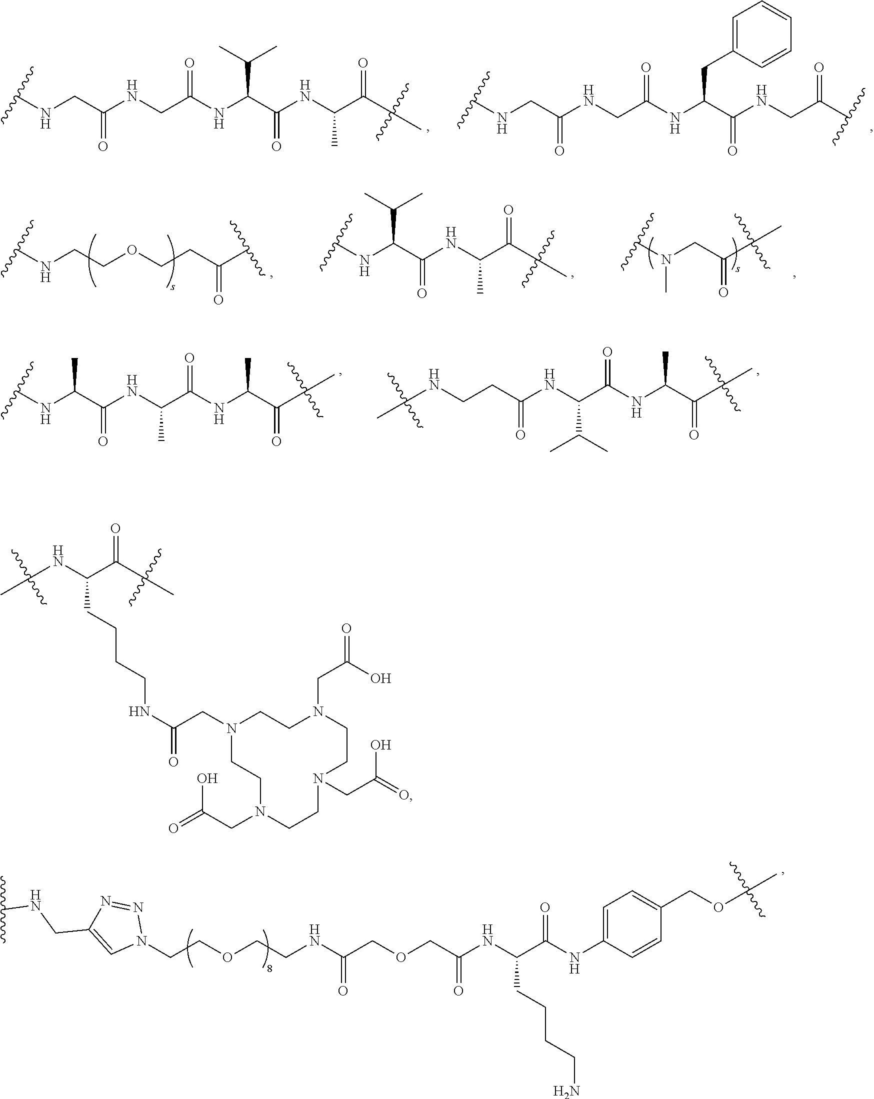

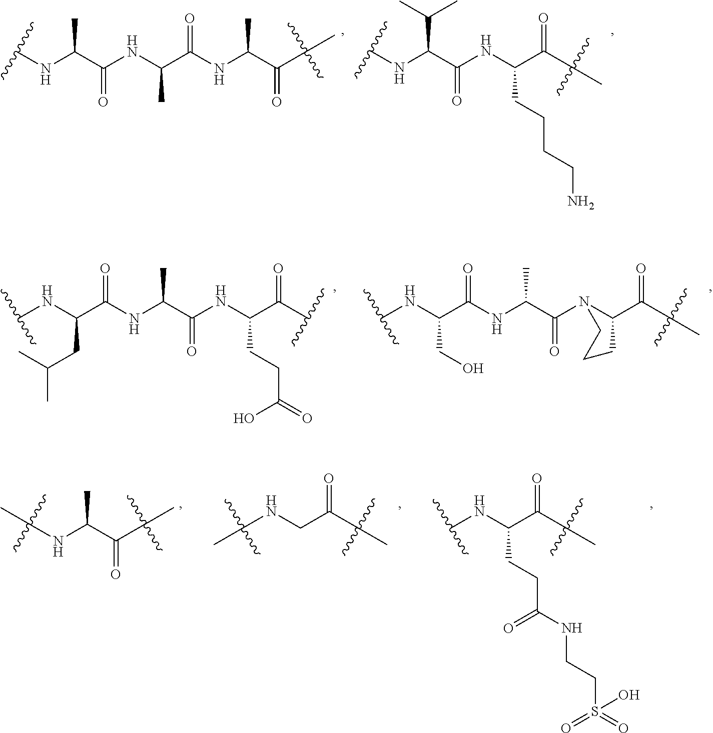

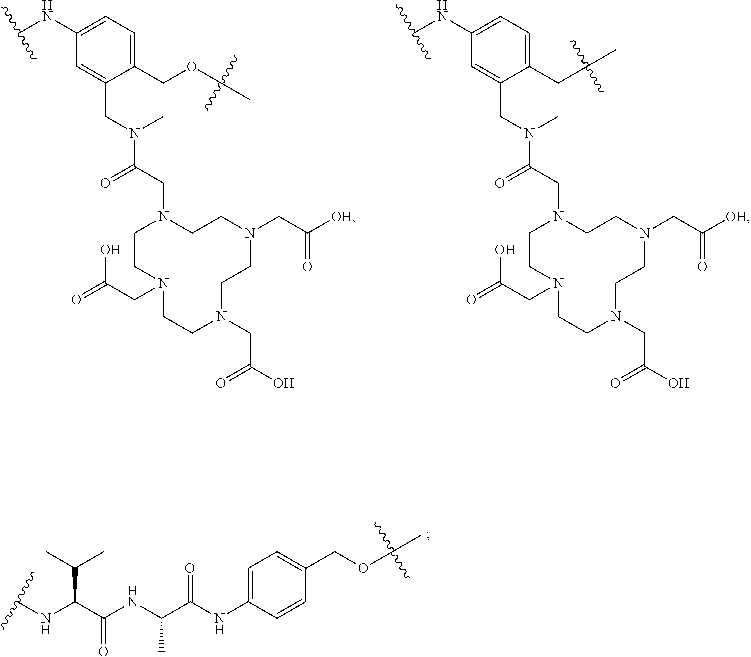 [SMILES: *Nc1ccc(CO*)c(CN(C)C(=O)CN2CCN(CC(=O)O)CCN(CC(=O)O)CCN(CC(=O)O)CC2)c1 ; *Cc1ccc(N*)cc1CN(C)C(=O)CN1CCN(CC(=O)O)CCN(CC(=O)O)CCN(CC(=O)O)CC1 ; *N[C@H](C(=O)N[C@@H](C)C(=O)Nc1ccc(CO*)cc1)C(C)C]

s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.

In some embodiments, L is selected from the group consisting of substituted or unsubstituted structural fragments composed of one or more of the following groups:

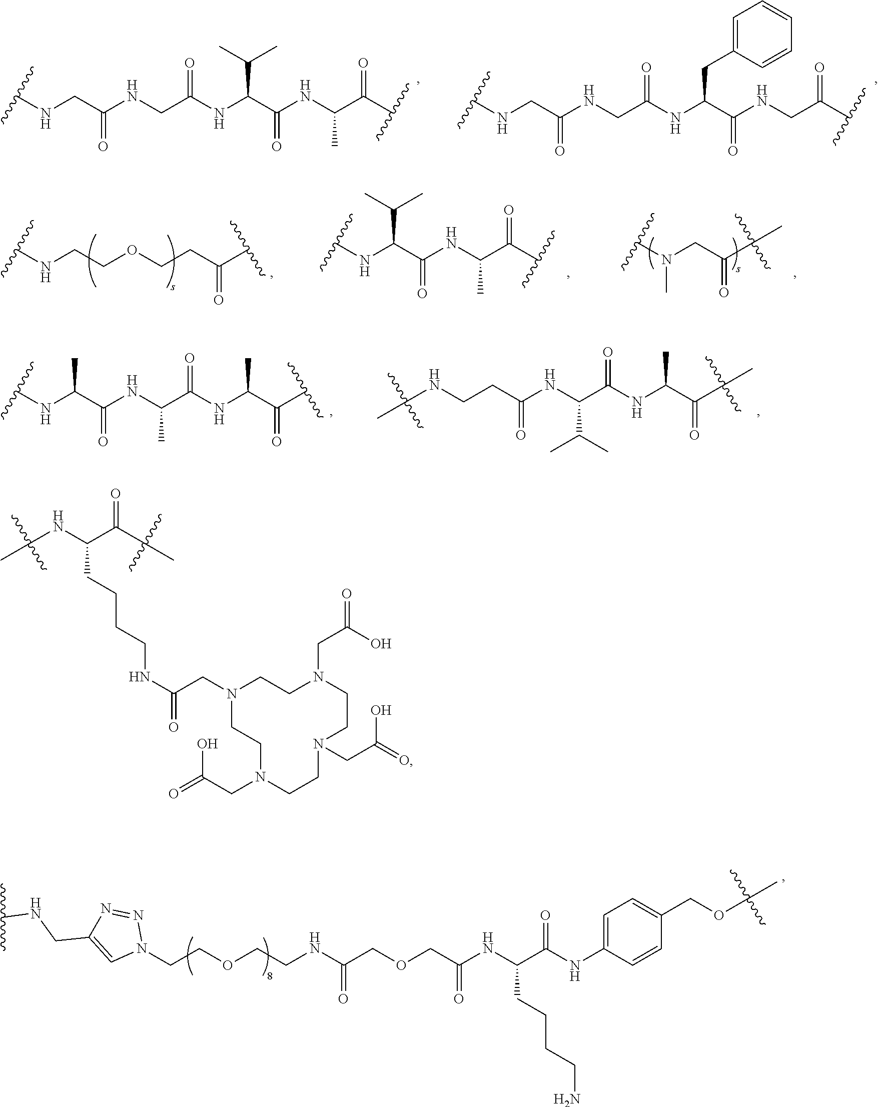

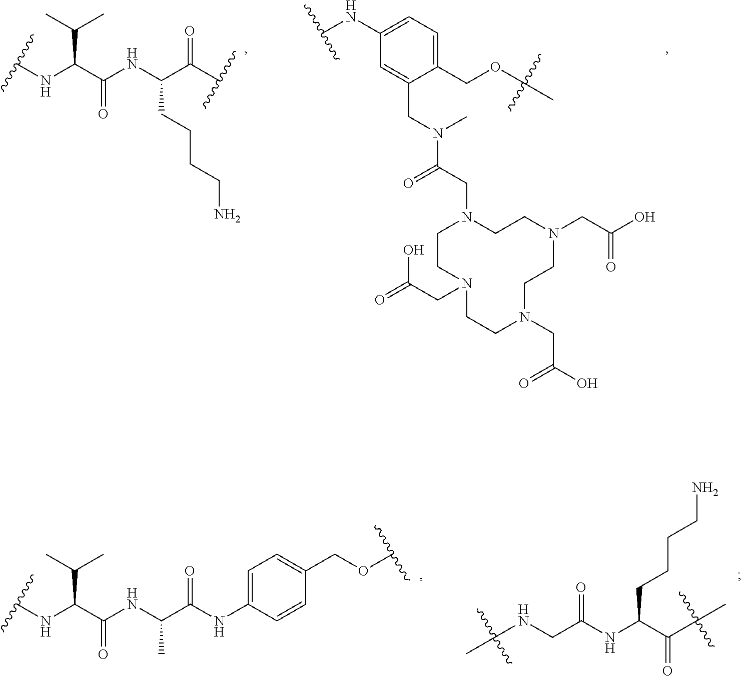 [SMILES: *N[C@H](C(=O)N[C@@H](CCCCN)C(*)=O)C(C)C ; *Nc1ccc(CO*)c(CN(C)C(=O)CN2CCN(CC(=O)O)CCN(CC(=O)O)CCN(CC(=O)O)CC2)c1 ; *N[C@H](C(=O)N[C@@H](C)C(=O)Nc1ccc(CO*)cc1)C(C)C ; *NCC(=O)N[C@@H](CCCCN)C(*)=O]

s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19, or 20.

In some embodiments, L is selected from the group consisting of substituted or unsubstituted structural fragments composed of one or more (e.g., 1, 2, 3, 4, or 5) of the following groups:

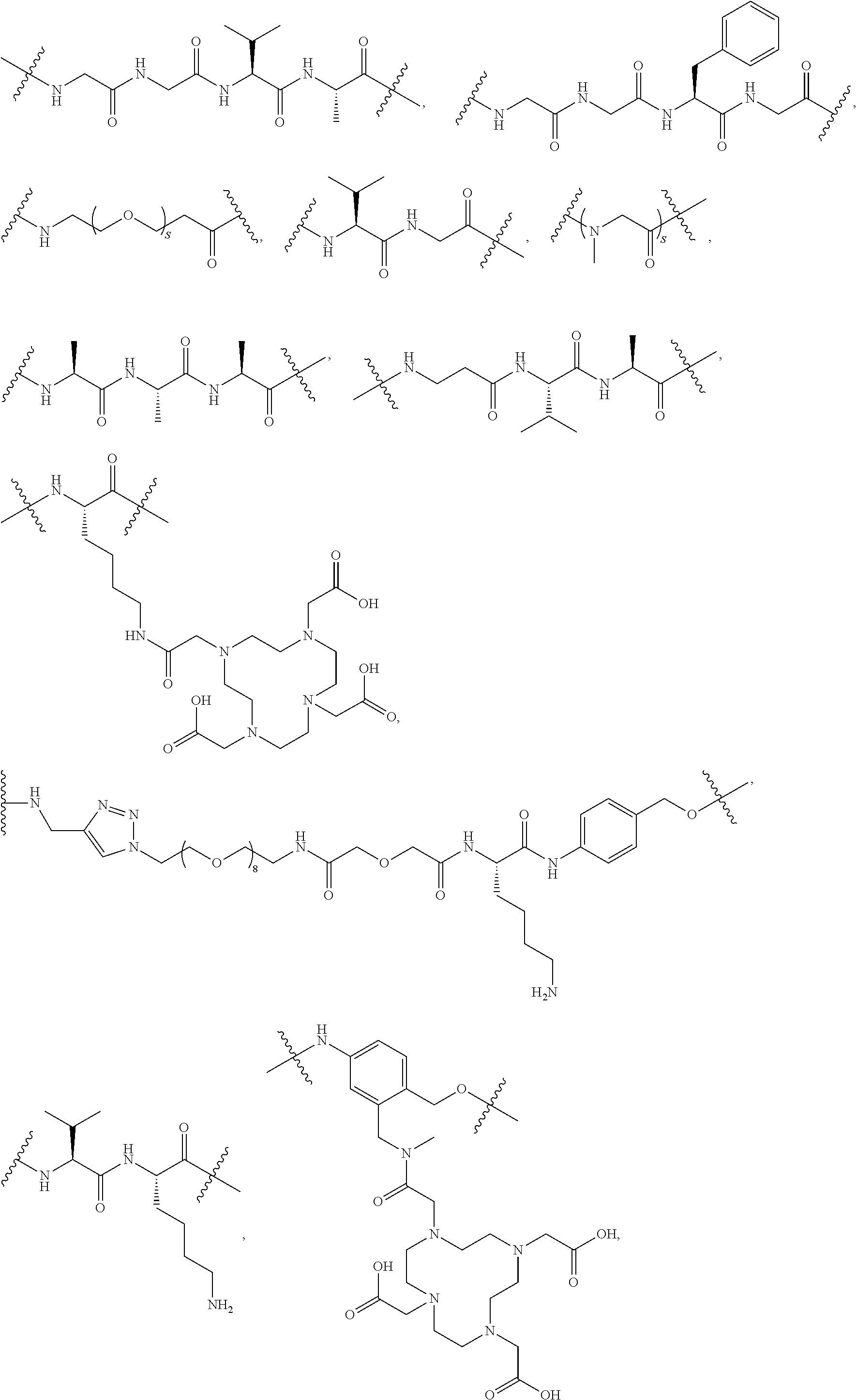

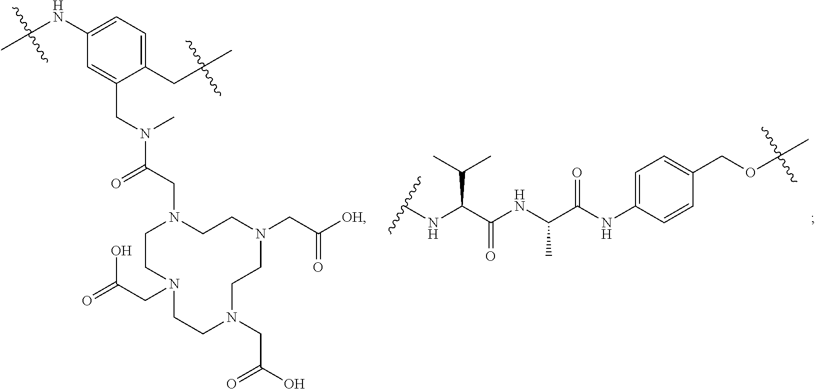 [SMILES: *Cc1ccc(N*)cc1CN(C)C(=O)CN1CCN(CC(=O)O)CCN(CC(=O)O)CCN(CC(=O)O)CC1 ; *N[C@H](C(=O)N[C@@H](C)C(=O)Nc1ccc(CO*)cc1)C(C)C]

s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19, or 20.

In some embodiments, L is selected from the group consisting of substituted or unsubstituted structural fragments composed of one or more (e.g., 1, 2, 3, 4, or 5) of the following groups:

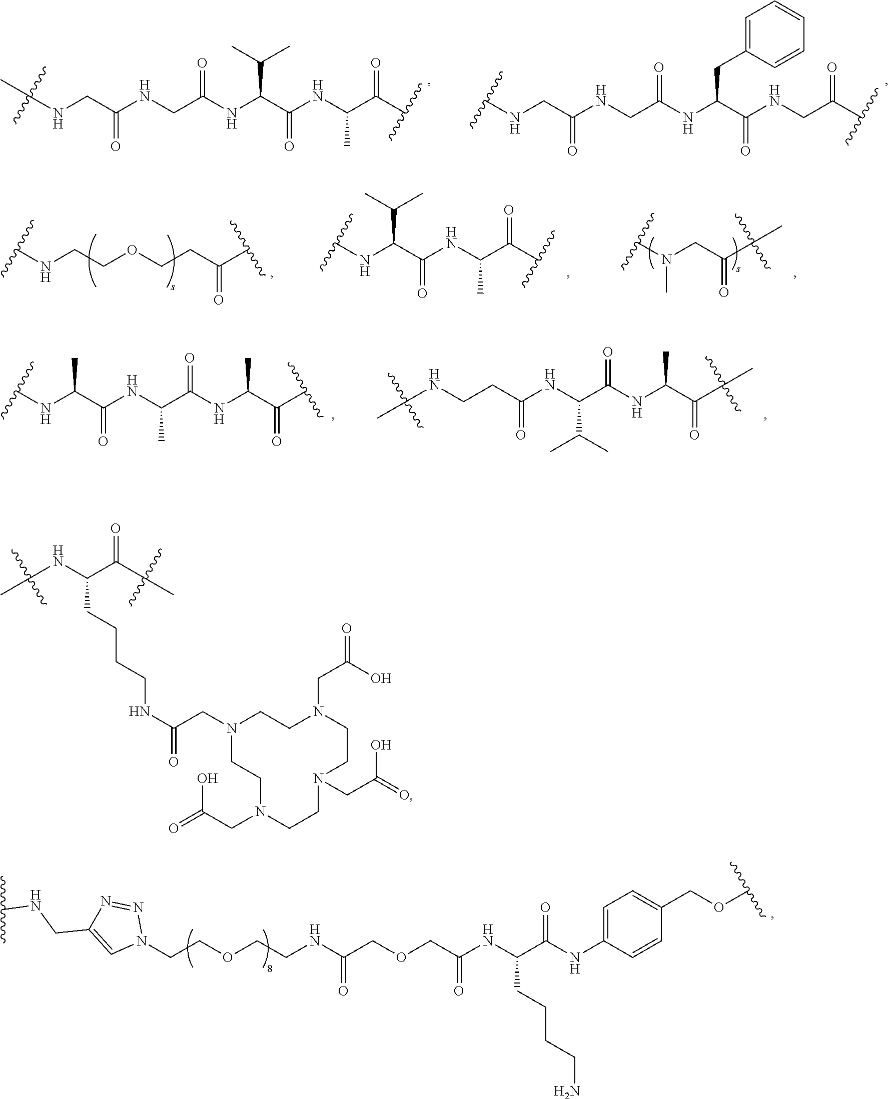

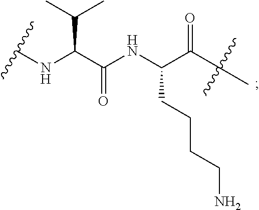 [SMILES: *N[C@H](C(=O)N[C@@H](CCCCN)C(*)=O)C(C)C]

s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19, or 20, preferably 8, 10.

In some embodiments, E is a single bond, substituted or unsubstituted —NH—CH₂—, or selected from the group consisting of the following substituted or unsubstituted structural fragments:

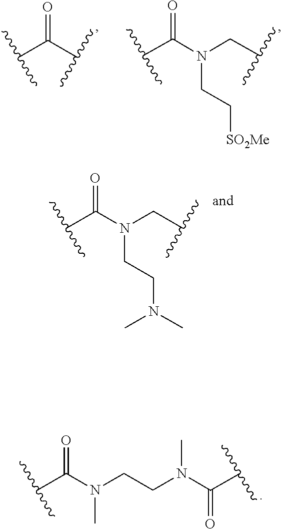

In some embodiments, E is a single bond, substituted or unsubstituted —NH—CH₂—,

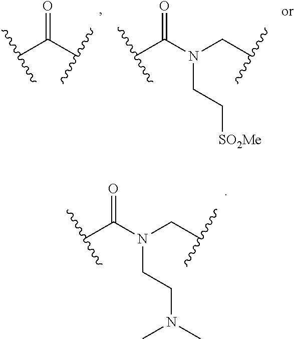

In some embodiments, E is a single bond, —NH—CH₂— or

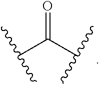 [SMILES: *C(*)=O ; "."]

In some embodiments, E is a single bond, substituted or unsubstituted —NH—CH₂— or

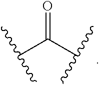 [SMILES: *C(*)=O ; "."]

In some embodiments, E is substituted or unsubstituted —NH—CH₂— or

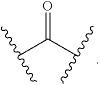 [SMILES: *C(*)=O ; "."]

In some embodiments,

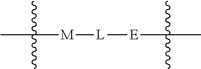

is selected from the group consisting of the following substituted or unsubstituted structural fragments:

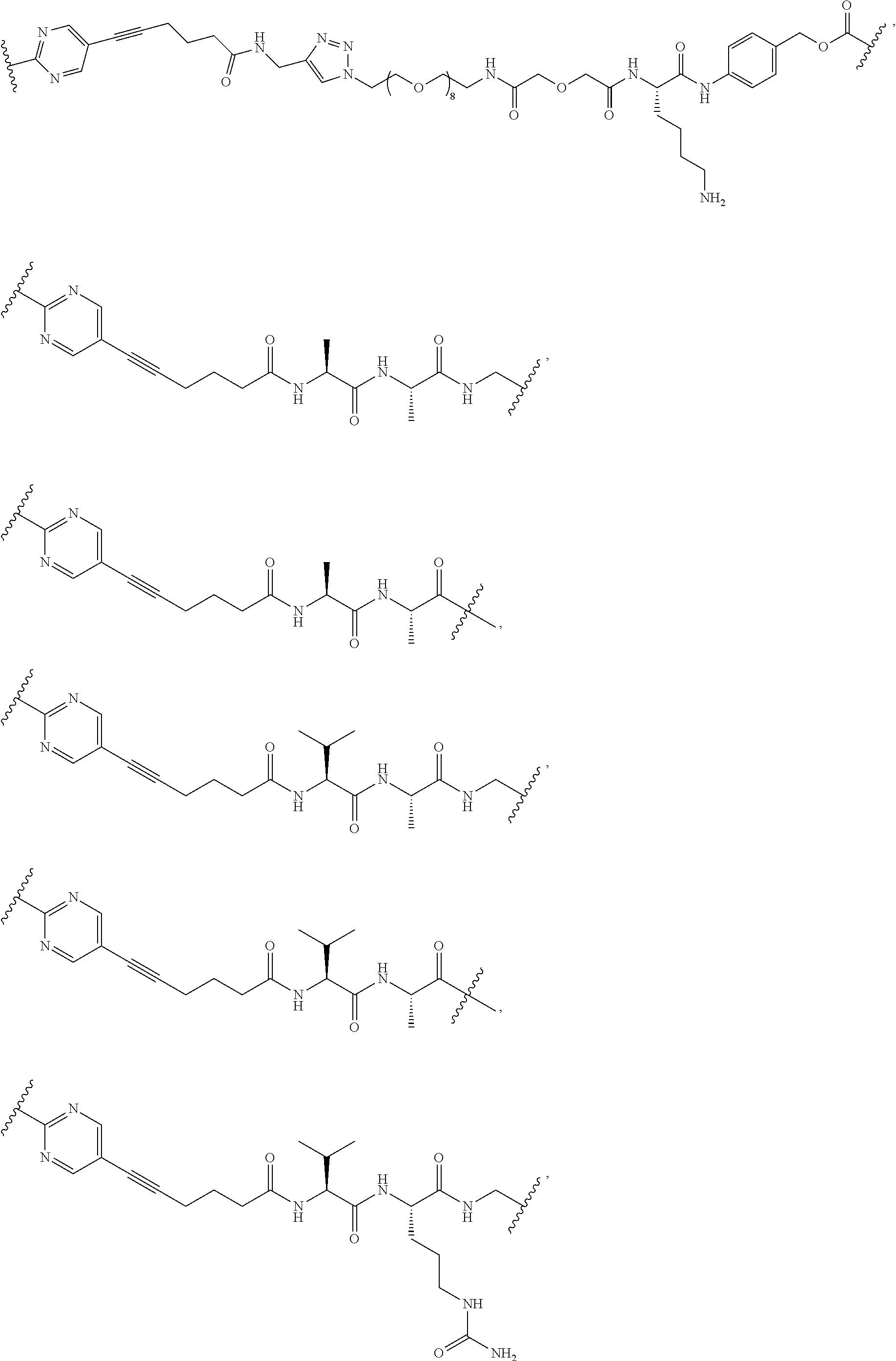

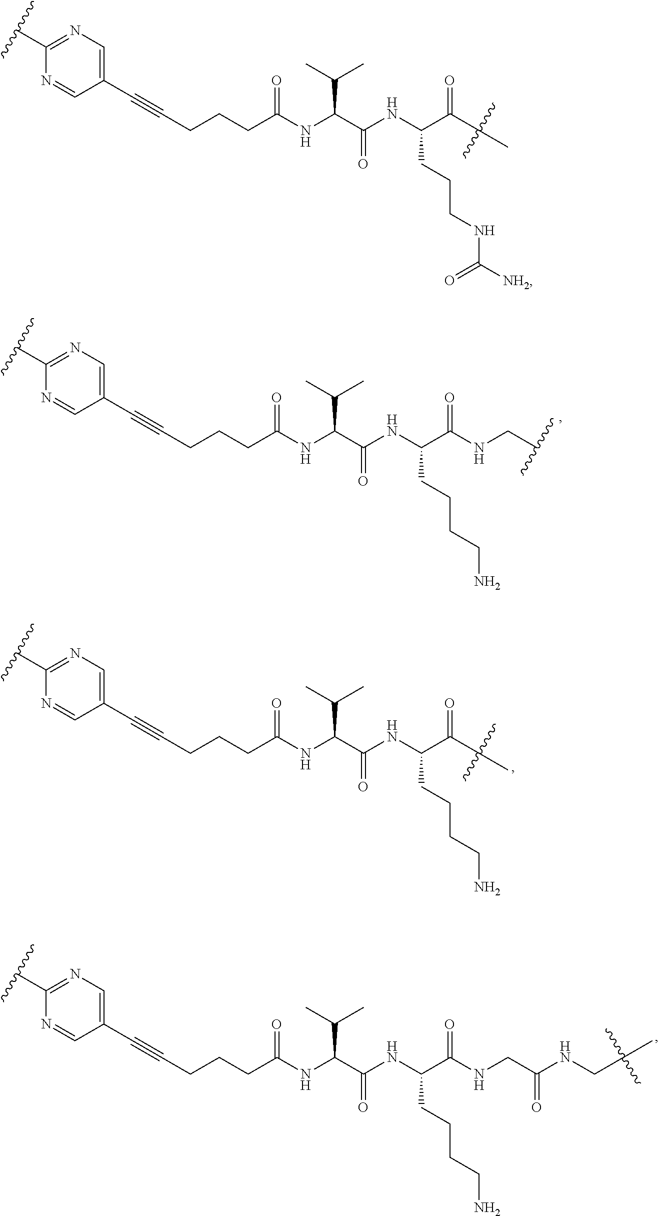 [SMILES: *C(=O)[C@H](CCCNC(N)=O)NC(=O)[C@@H](NC(=O)CCCC#Cc1cnc(*)nc1)C(C)C ; *CNC(=O)[C@H](CCCCN)NC(=O)[C@@H](NC(=O)CCCC#Cc1cnc(*)nc1)C(C)C ; *C(=O)[C@H](CCCCN)NC(=O)[C@@H](NC(=O)CCCC#Cc1cnc(*)nc1)C(C)C ; *CNC(=O)CNC(=O)[C@H](CCCCN)NC(=O)[C@@H](NC(=O)CCCC#Cc1cnc(*)nc1)C(C)C]

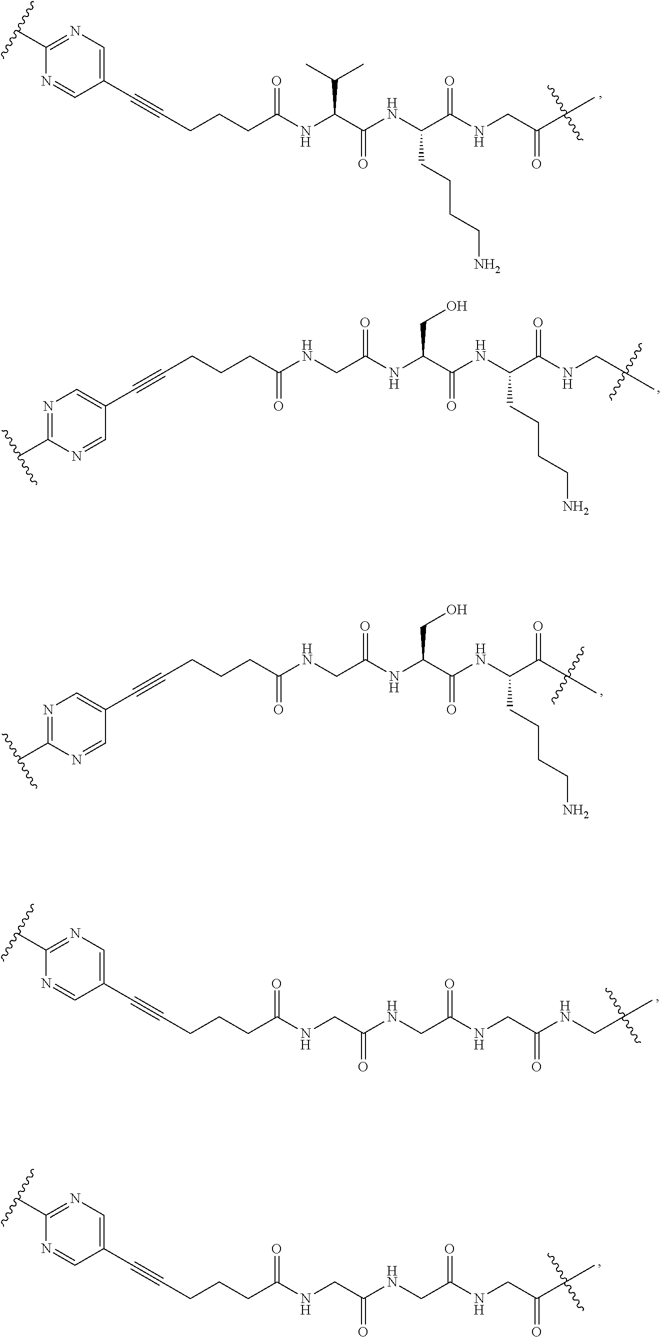 [SMILES: *C(=O)CNC(=O)[C@H](CCCCN)NC(=O)[C@@H](NC(=O)CCCC#Cc1cnc(*)nc1)C(C)C ; *CNC(=O)[C@H](CCCCN)NC(=O)[C@H](CO)NC(=O)CNC(=O)CCCC#Cc1cnc(*)nc1 ; *C(=O)[C@H](CCCCN)NC(=O)[C@H](CO)NC(=O)CNC(=O)CCCC#Cc1cnc(*)nc1 ; *CNC(=O)CNC(=O)CNC(=O)CNC(=O)CCCC#Cc1cnc(*)nc1 ; *C(=O)CNC(=O)CNC(=O)CNC(=O)CCCC#Cc1cnc(*)nc1]

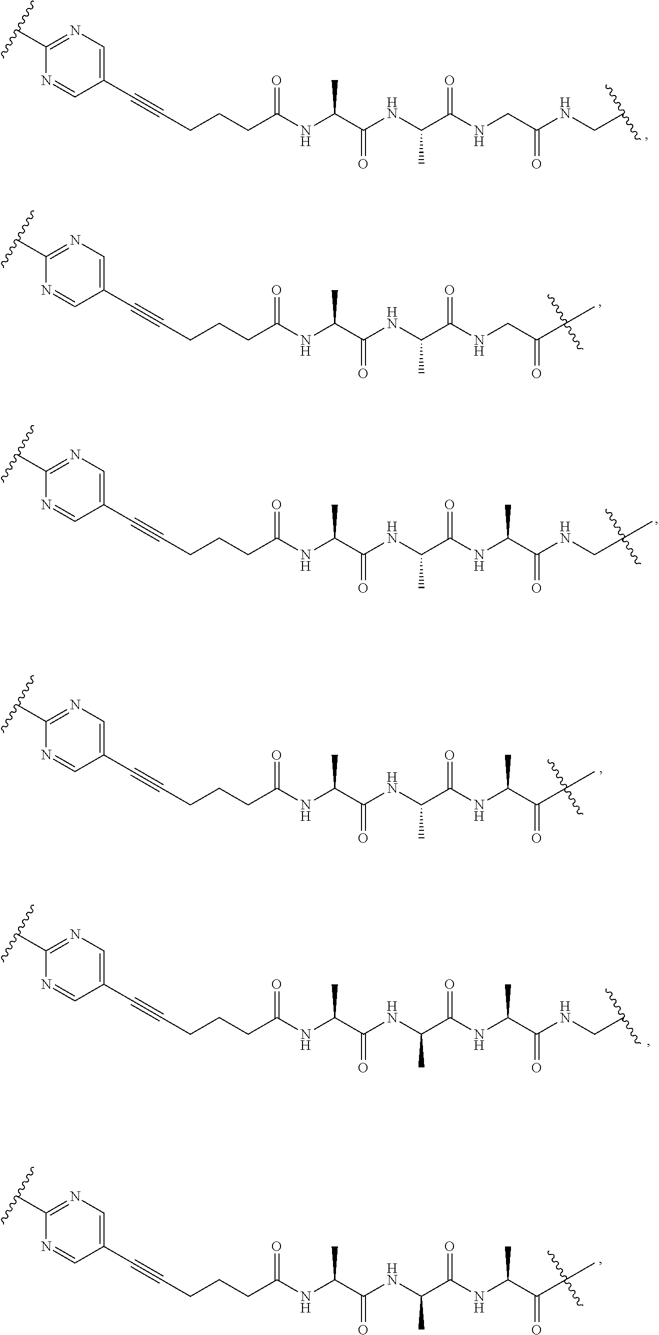 [SMILES: *CNC(=O)CNC(=O)[C@H](C)NC(=O)[C@H](C)NC(=O)CCCC#Cc1cnc(*)nc1 ; *C(=O)CNC(=O)[C@H](C)NC(=O)[C@H](C)NC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)[C@H](C)NC(=O)[C@H](C)NC(=O)[C@H](C)NC(=O)CCCC#Cc1cnc(*)nc1 ; *CNC(=O)[C@H](C)NC(=O)[C@@H](C)NC(=O)[C@H](C)NC(=O)CCCC#Cc1cnc(*)nc1 ; *C(=O)[C@H](C)NC(=O)[C@@H](C)NC(=O)[C@H](C)NC(=O)CCCC#Cc1cnc(*)nc1]

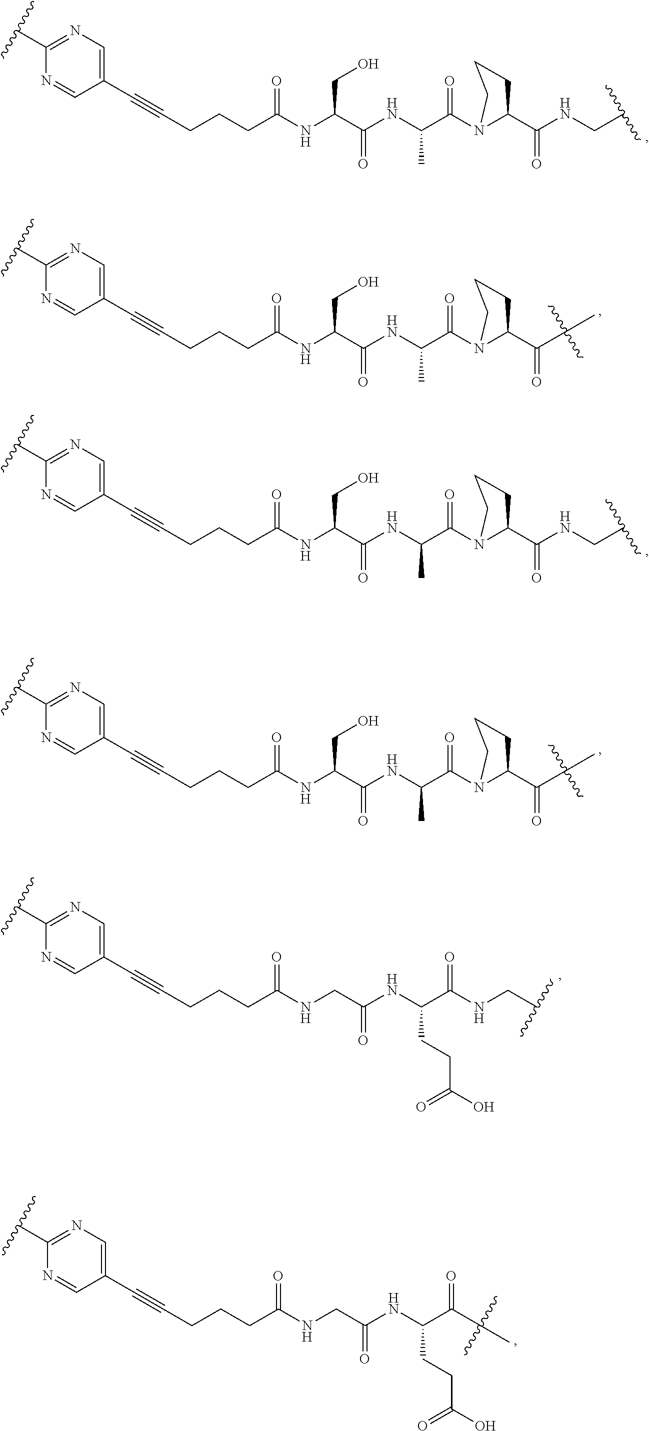

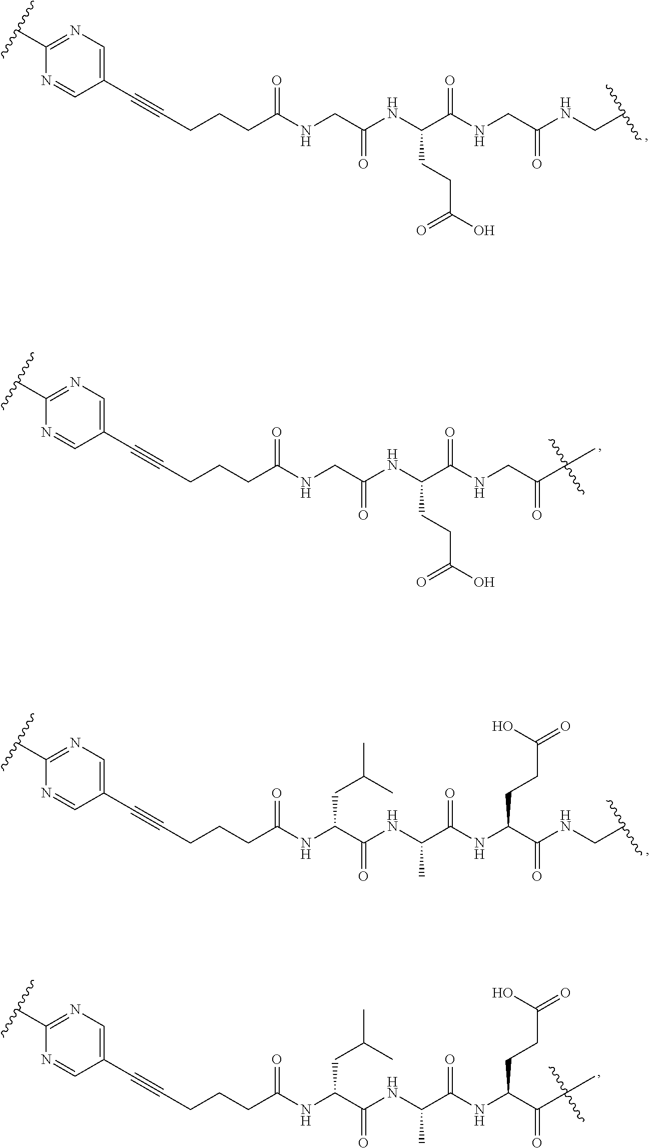 [SMILES: *CNC(=O)CNC(=O)[C@H](CCC(=O)O)NC(=O)CNC(=O)CCCC#Cc1cnc(*)nc1 ; *C(=O)CNC(=O)[C@H](CCC(=O)O)NC(=O)CNC(=O)CCCC#Cc1cnc(*)nc1 ; *CNC(=O)[C@H](CCC(=O)O)NC(=O)[C@H](C)NC(=O)[C@@H](CC(C)C)NC(=O)CCCC#Cc1cnc(*)nc1 ; *C(=O)[C@H](CCC(=O)O)NC(=O)[C@H](C)NC(=O)[C@@H](CC(C)C)NC(=O)CCCC#Cc1cnc(*)nc1]

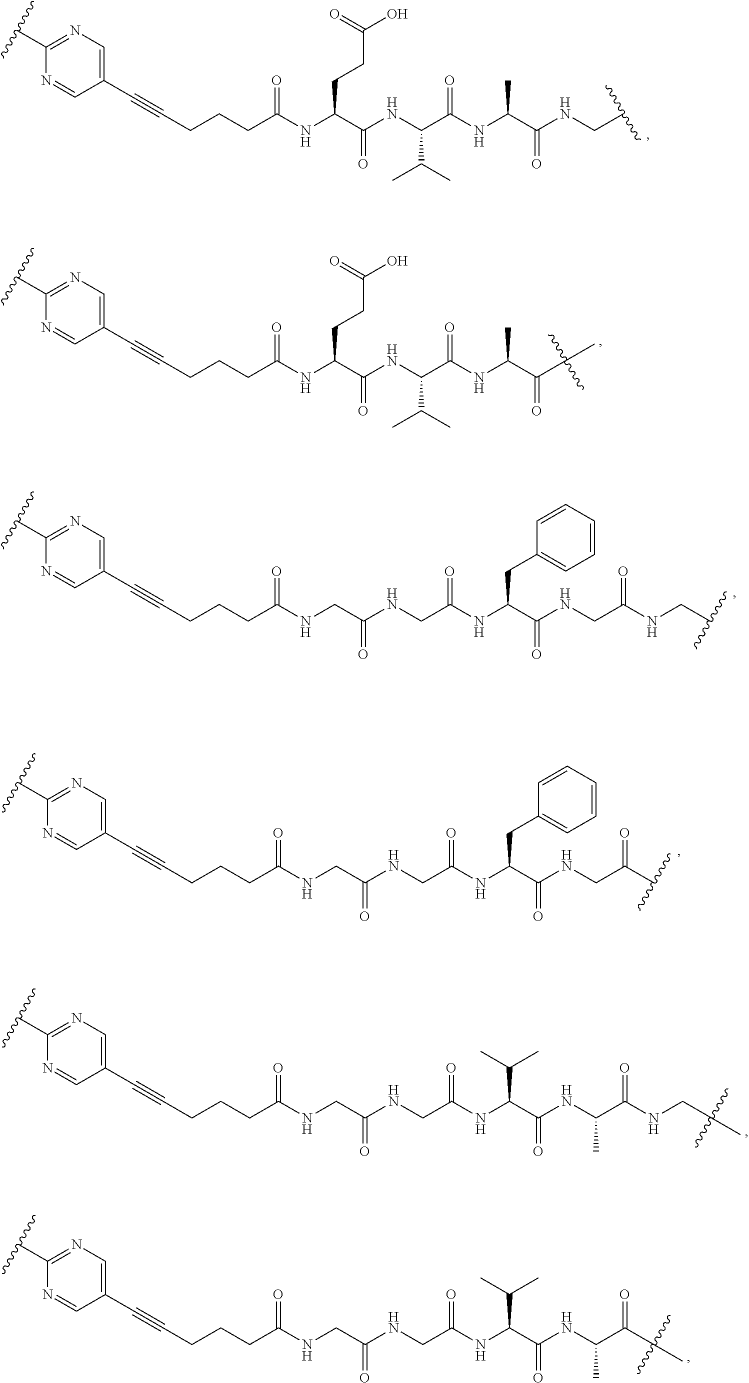 [SMILES: *CNC(=O)[C@H](C)NC(=O)[C@@H](NC(=O)[C@H](CCC(=O)O)NC(=O)CCCC#Cc1cnc(*)nc1)C(C)C ; *C(=O)[C@H](C)NC(=O)[C@@H](NC(=O)[C@H](CCC(=O)O)NC(=O)CCCC#Cc1cnc(*)nc1)C(C)C ; *CNC(=O)CNC(=O)[C@H](Cc1ccccc1)NC(=O)CNC(=O)CNC(=O)CCCC#Cc1cnc(*)nc1 ; *C(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@@H](NC(=O)CNC(=O)CNC(=O)CCCC#Cc1cnc(*)nc1)C(C)C ; *C(=O)[C@H](C)NC(=O)[C@@H](NC(=O)CNC(=O)CNC(=O)CCCC#Cc1cnc(*)nc1)C(C)C]

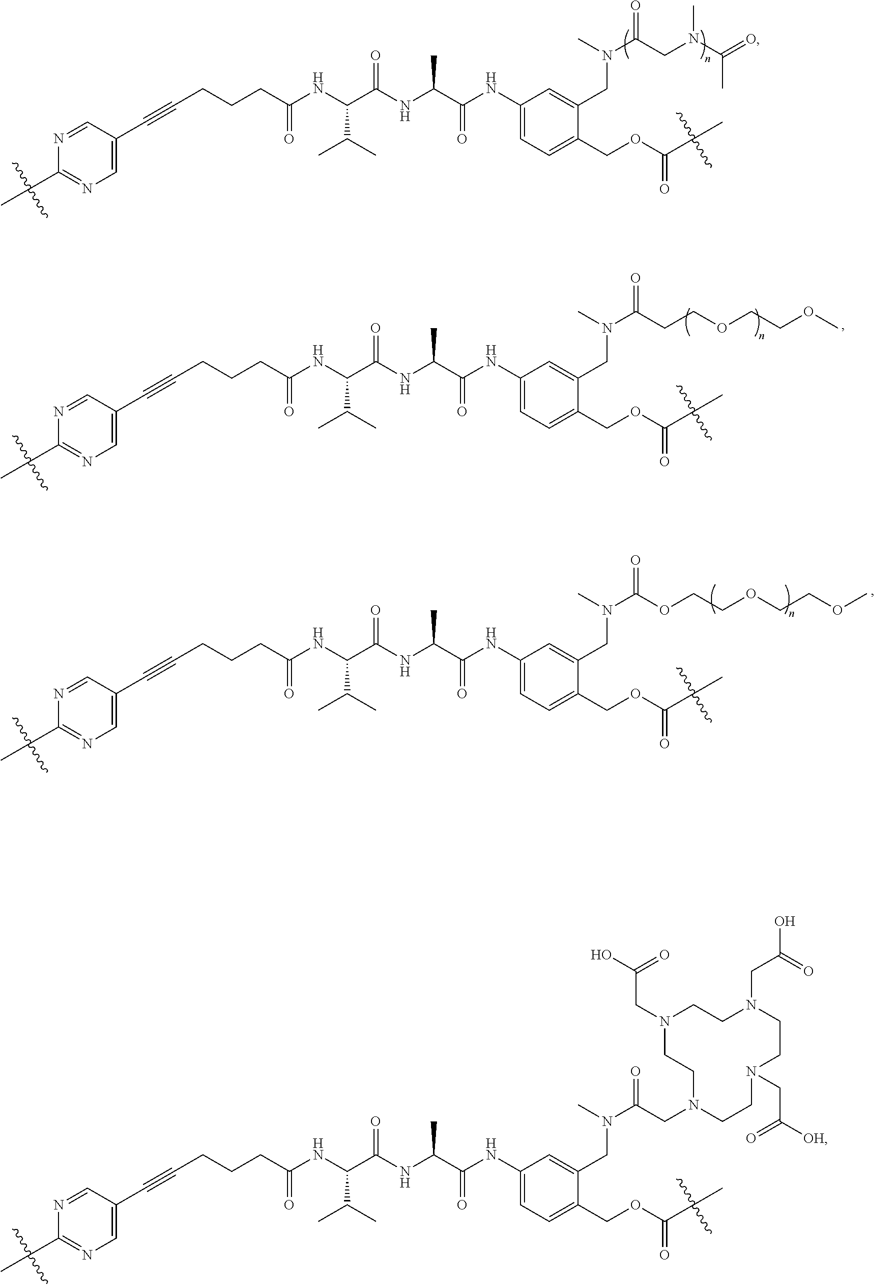

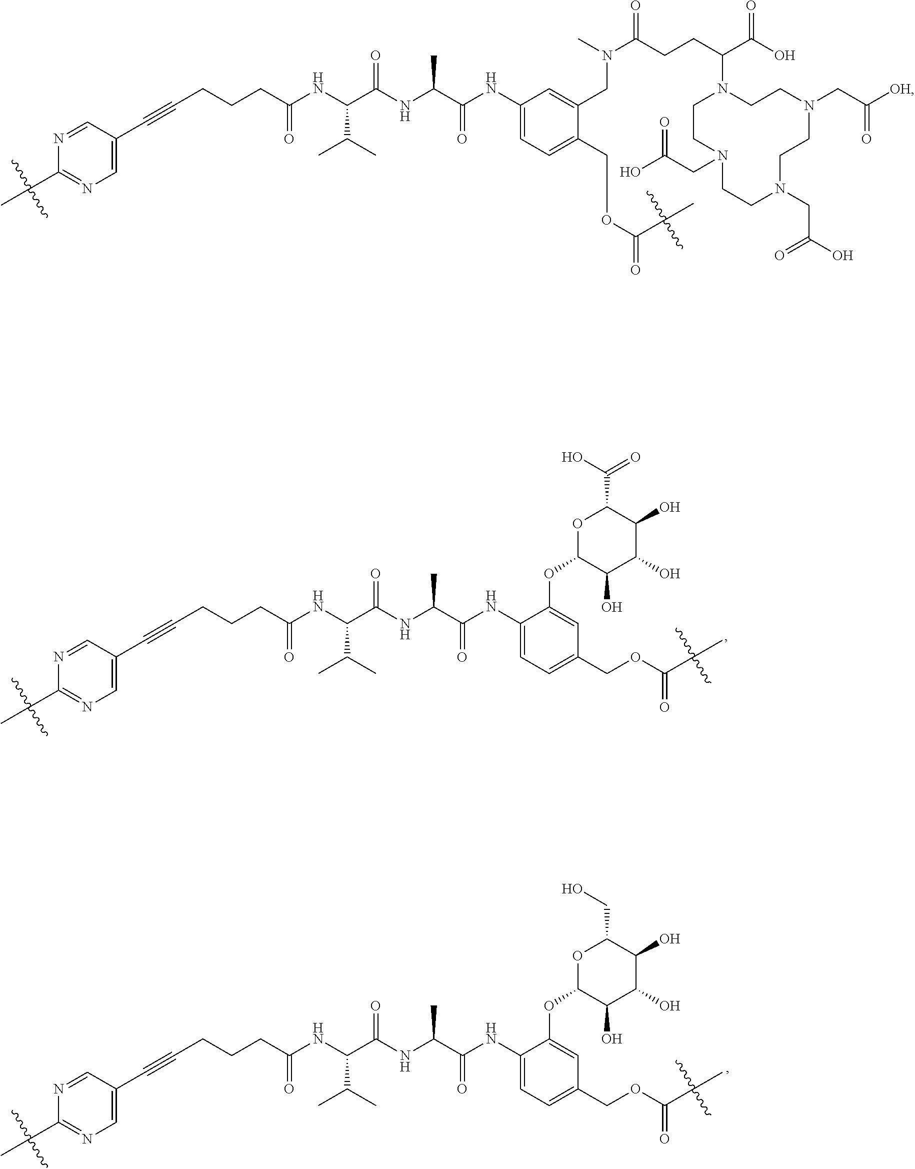 [SMILES: *C(=O)OCc1ccc(NC(=O)[C@H](C)NC(=O)[C@@H](NC(=O)CCCC#Cc2cnc(*)nc2)C(C)C)cc1CN(C)C(=O)CCC(C(=O)O)N1CCN(CC(=O)O)CCN(CC(=O)O)CCN(CC(=O)O)CC1 ; *C(=O)OCc1ccc(NC(=O)[C@H](C)NC(=O)[C@@H](NC(=O)CCCC#Cc2cnc(*)nc2)C(C)C)c(O[C@@H]2O[C@H](C(=O)O)[C@@H](O)[C@H](O)[C@H]2O)c1 ; *C(=O)OCc1ccc(NC(=O)[C@H](C)NC(=O)[C@@H](NC(=O)CCCC#Cc2cnc(*)nc2)C(C)C)c(O[C@@H]2O[C@H](CO)[C@@H](O)[C@H](O)[C@H]2O)c1]

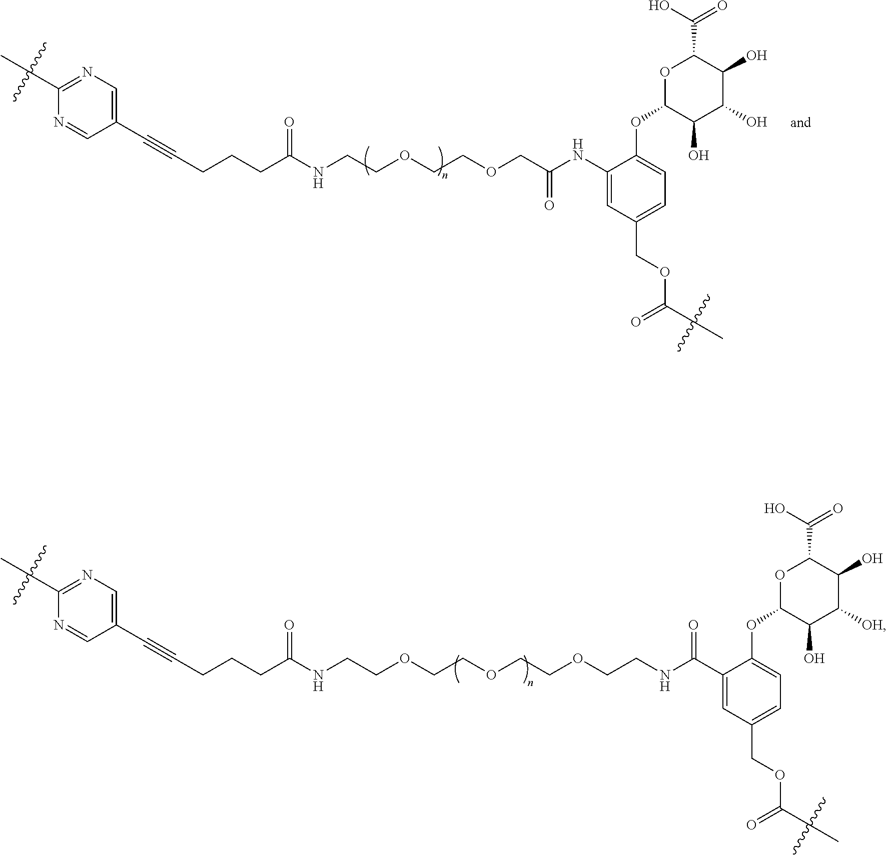

n is an integer selected from 1 to 20.

In some embodiments,

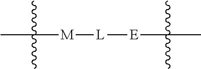

is selected from the group consisting of the following substituted or unsubstituted structural fragments:

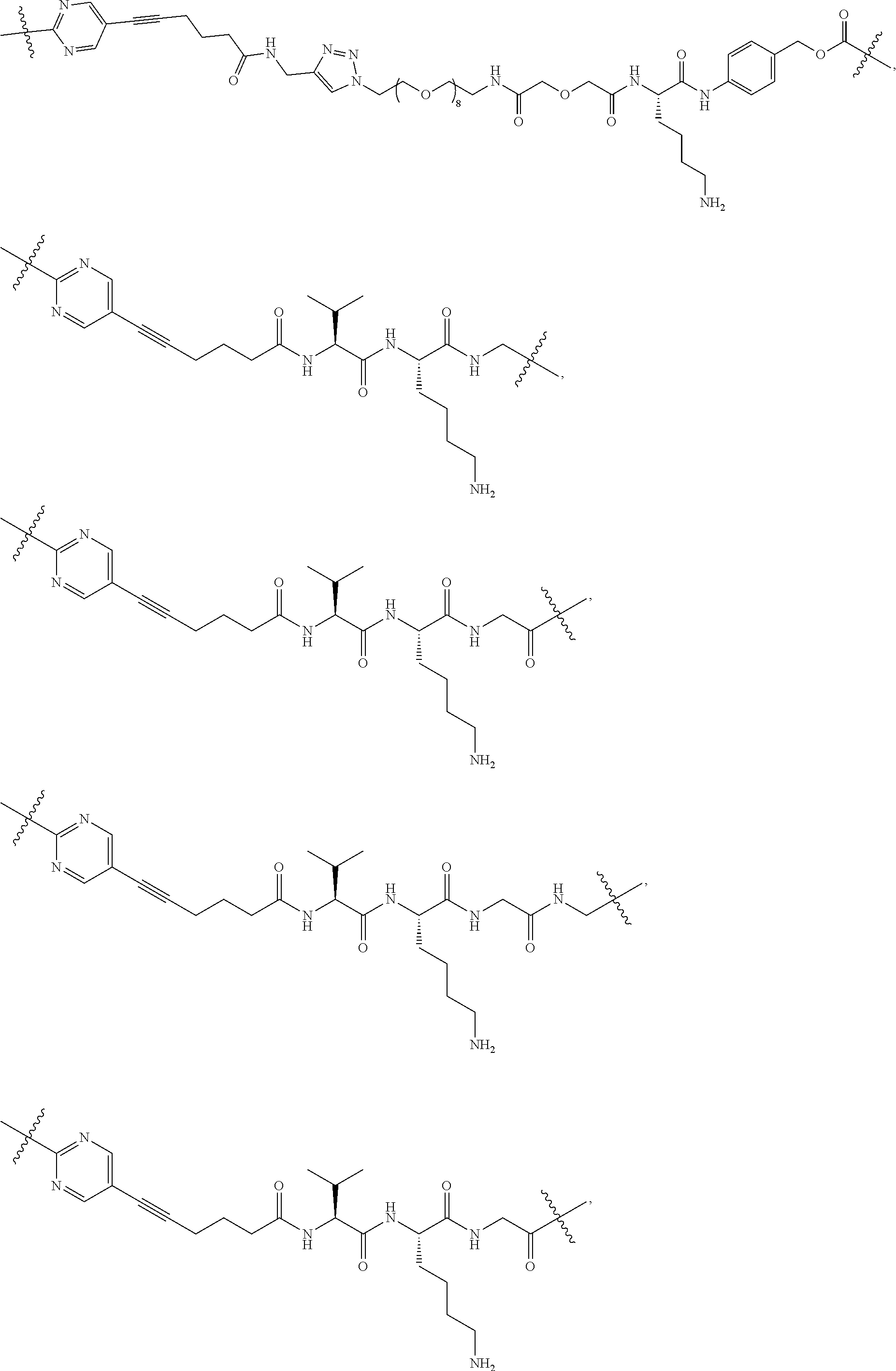

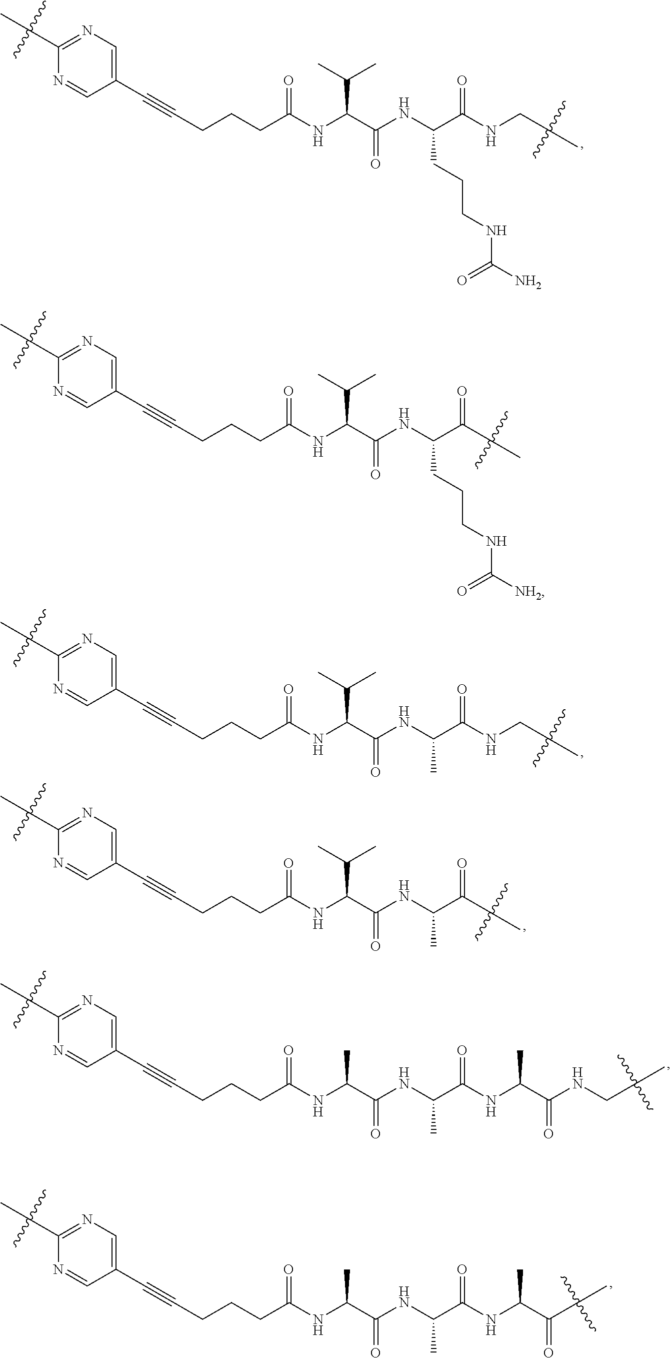 [SMILES: *CNC(=O)[C@H](CCCNC(N)=O)NC(=O)[C@@H](NC(=O)CCCC#Cc1cnc(*)nc1)C(C)C ; *C(=O)[C@H](CCCNC(N)=O)NC(=O)[C@@H](NC(=O)CCCC#Cc1cnc(*)nc1)C(C)C ; *CNC(=O)[C@H](C)NC(=O)[C@@H](NC(=O)CCCC#Cc1cnc(*)nc1)C(C)C ; *C(=O)[C@H](C)NC(=O)[C@@H](NC(=O)CCCC#Cc1cnc(*)nc1)C(C)C ; *CNC(=O)[C@H](C)NC(=O)[C@H](C)NC(=O)[C@H](C)NC(=O)CCCC#Cc1cnc(*)nc1 ; *C(=O)[C@H](C)NC(=O)[C@H](C)NC(=O)[C@H](C)NC(=O)CCCC#Cc1cnc(*)nc1]

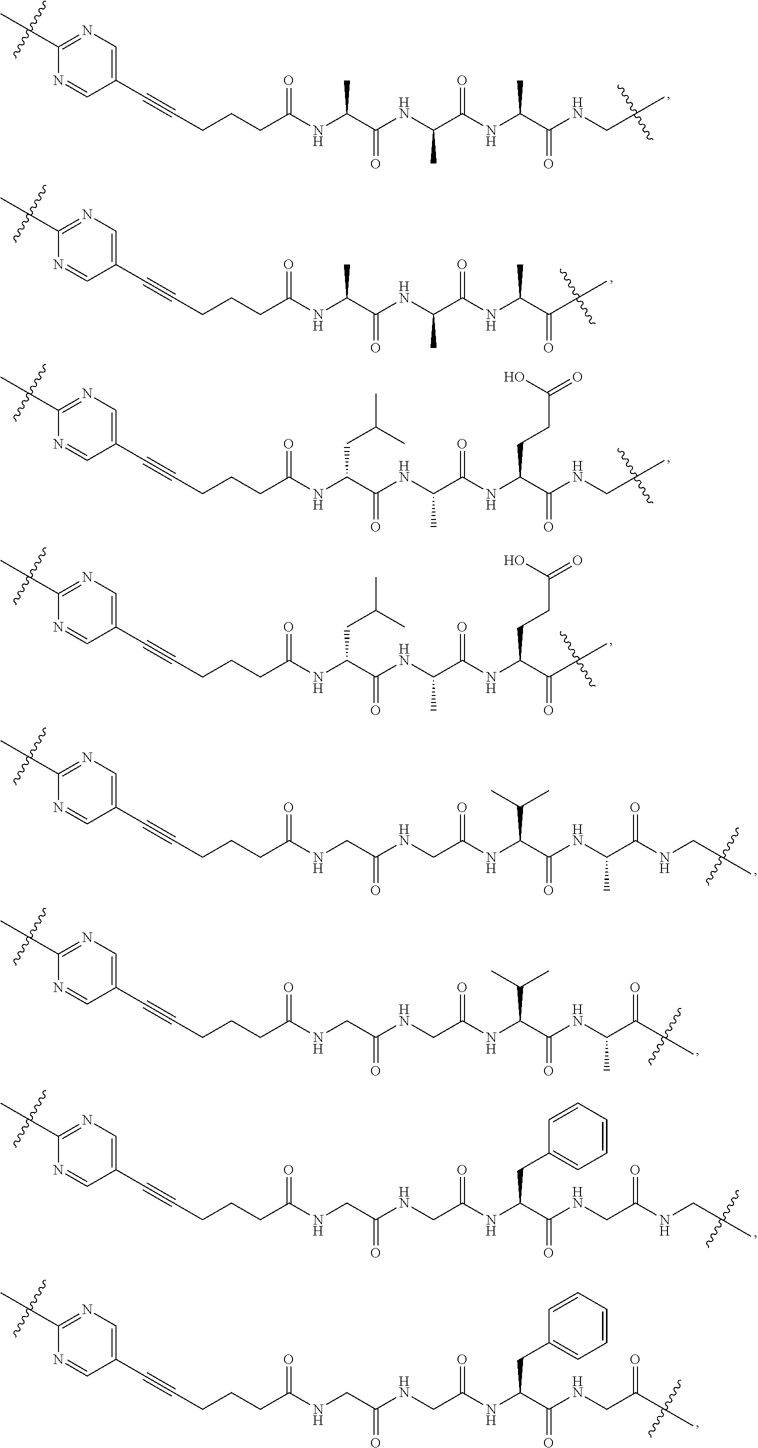

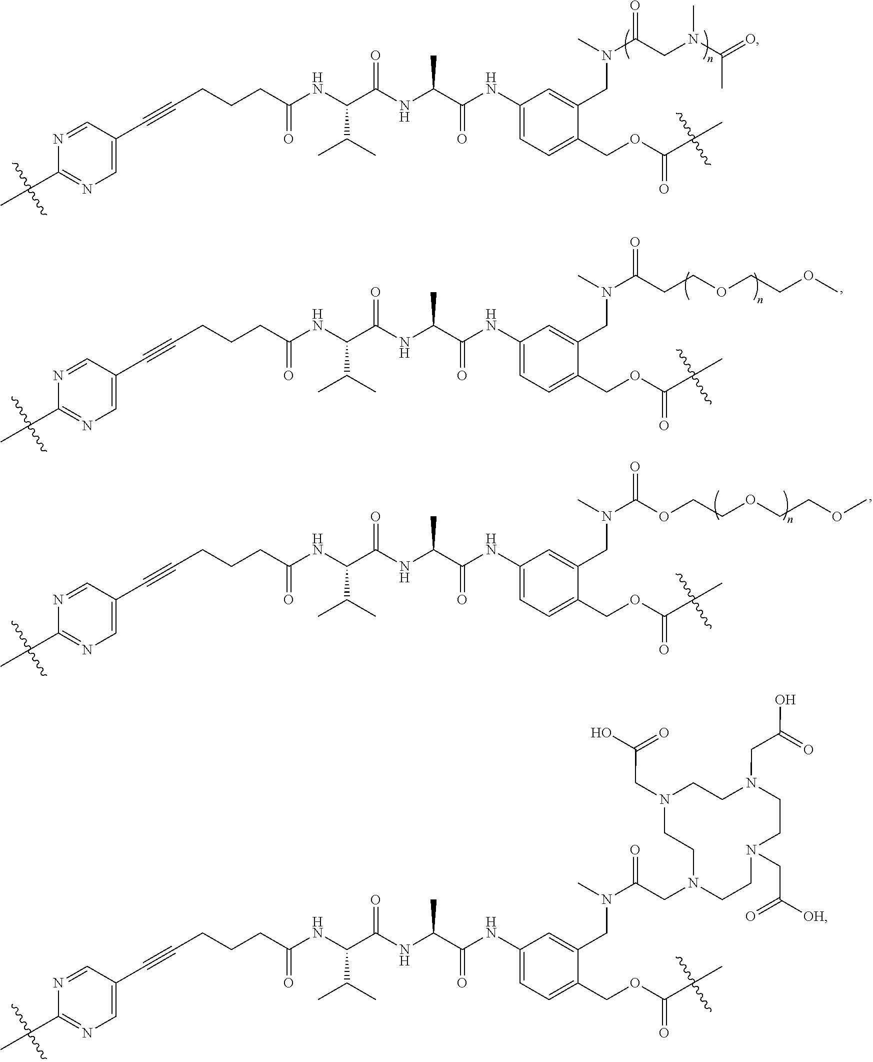

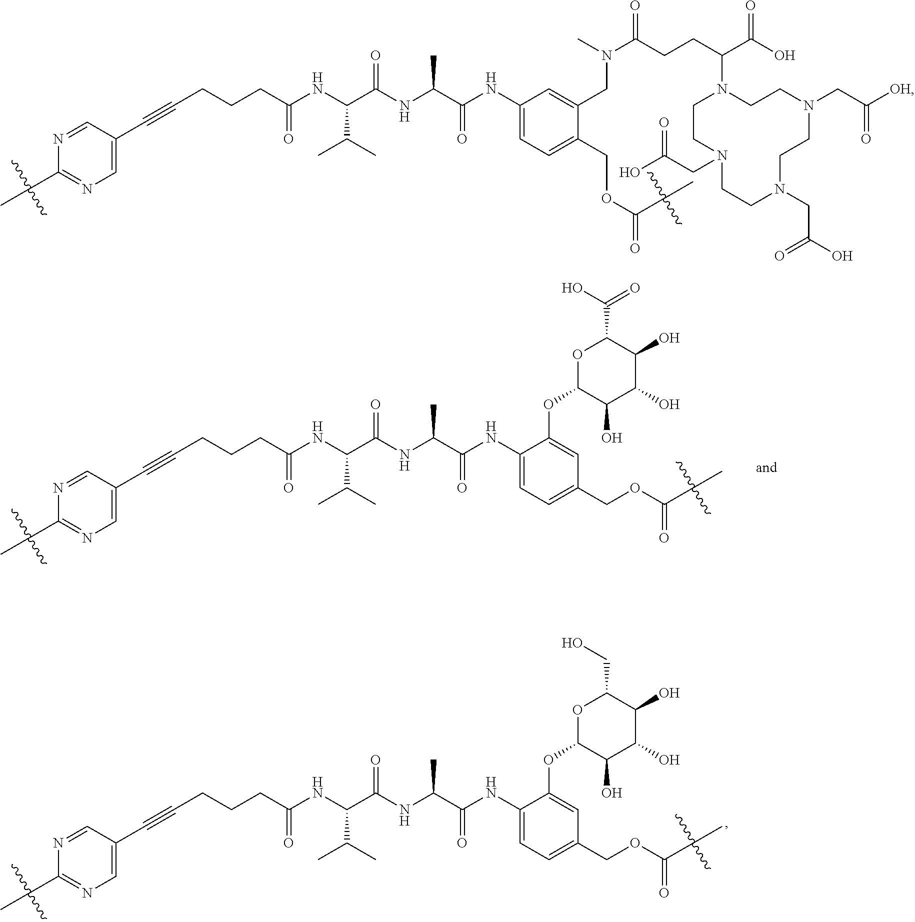

n is an integer selected from 1 to 20.

In some embodiments,

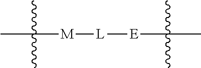

is selected from the group consisting of the following substituted or unsubstituted structural fragments:

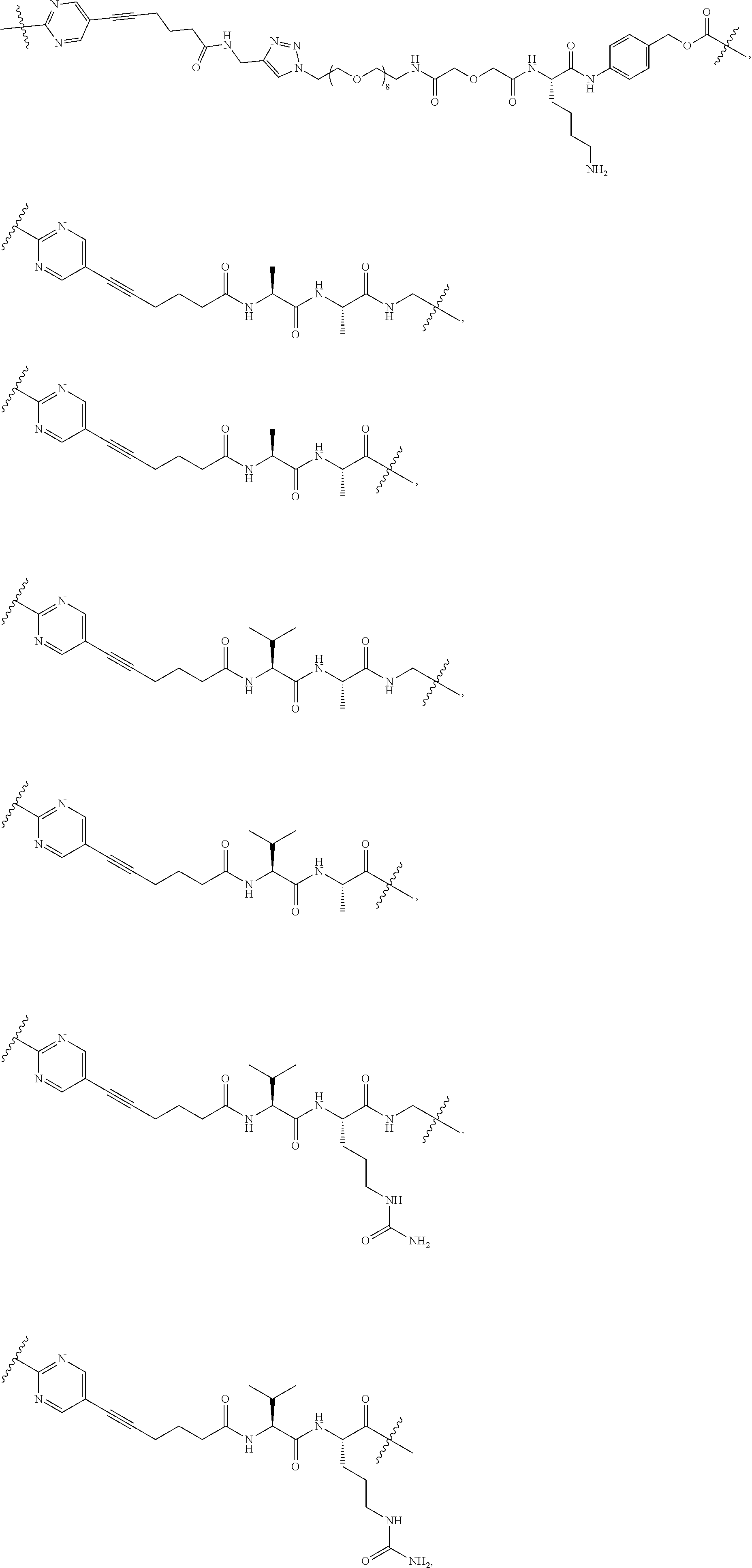

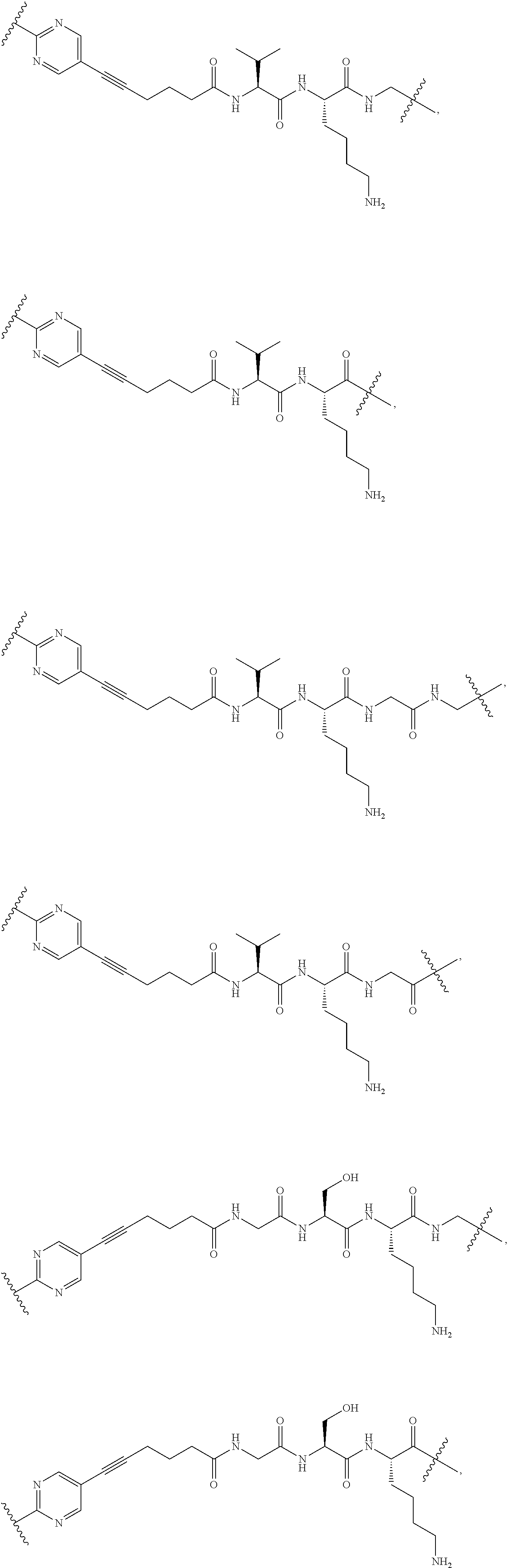 [SMILES: *CNC(=O)[C@H](CCCCN)NC(=O)[C@@H](NC(=O)CCCC#Cc1cnc(*)nc1)C(C)C ; *C(=O)[C@H](CCCCN)NC(=O)[C@@H](NC(=O)CCCC#Cc1cnc(*)nc1)C(C)C ; *CNC(=O)CNC(=O)[C@H](CCCCN)NC(=O)[C@@H](NC(=O)CCCC#Cc1cnc(*)nc1)C(C)C ; *C(=O)CNC(=O)[C@H](CCCCN)NC(=O)[C@@H](NC(=O)CCCC#Cc1cnc(*)nc1)C(C)C ; *CNC(=O)[C@H](CCCCN)NC(=O)[C@H](CO)NC(=O)CNC(=O)CCCC#Cc1cnc(*)nc1 ; *C(=O)[C@H](CCCCN)NC(=O)[C@H](CO)NC(=O)CNC(=O)CCCC#Cc1cnc(*)nc1]

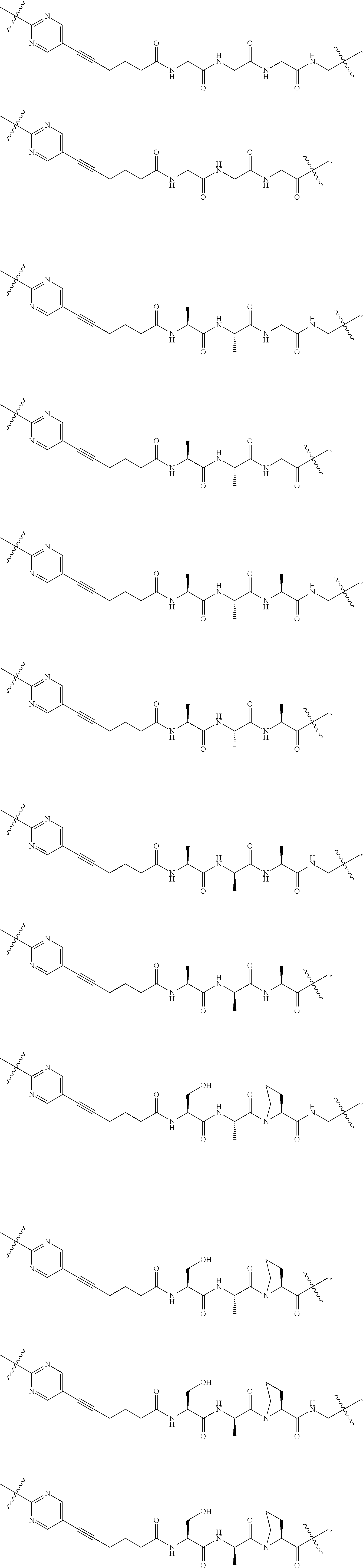

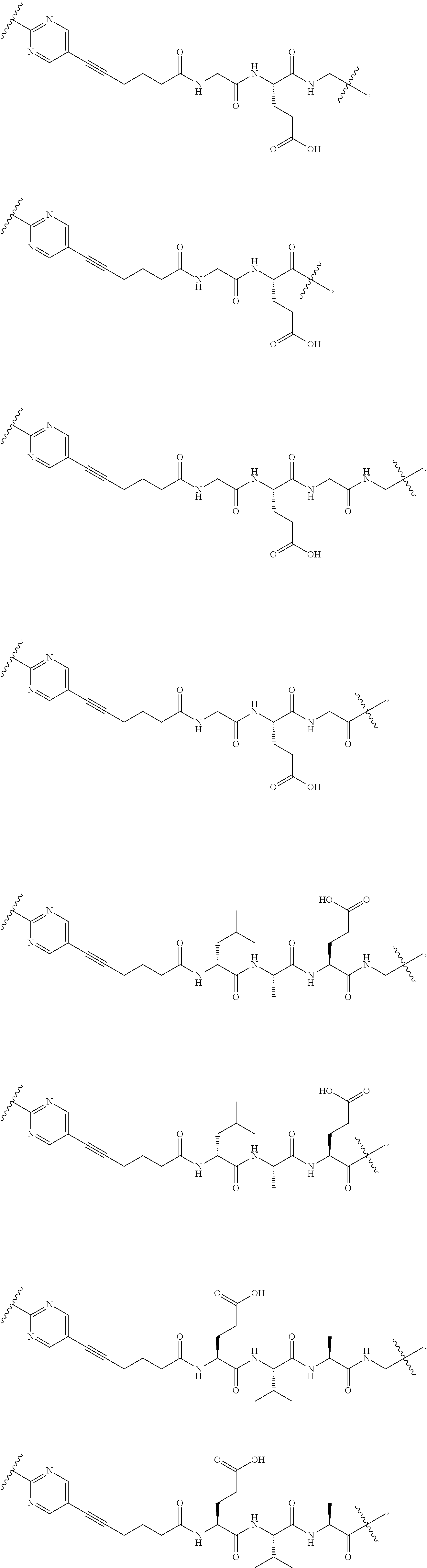

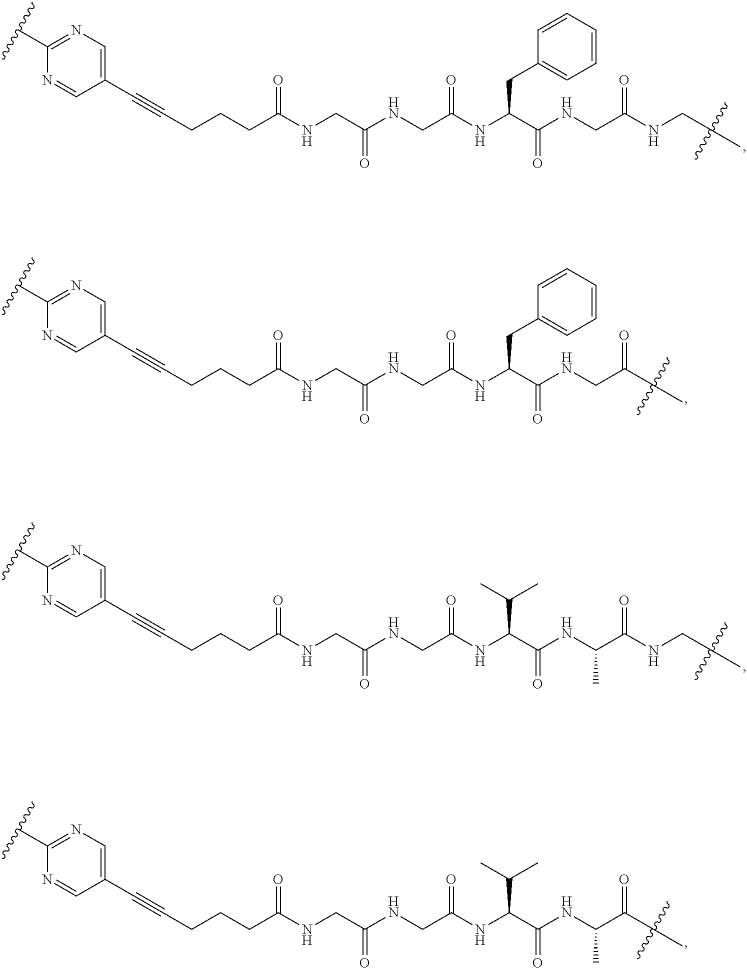 [SMILES: *CNC(=O)CNC(=O)[C@H](Cc1ccccc1)NC(=O)CNC(=O)CNC(=O)CCCC#Cc1cnc(*)nc1 ; *C(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@@H](NC(=O)CNC(=O)CNC(=O)CCCC#Cc1cnc(*)nc1)C(C)C ; *C(=O)[C@H](C)NC(=O)[C@@H](NC(=O)CNC(=O)CNC(=O)CCCC#Cc1cnc(*)nc1)C(C)C]

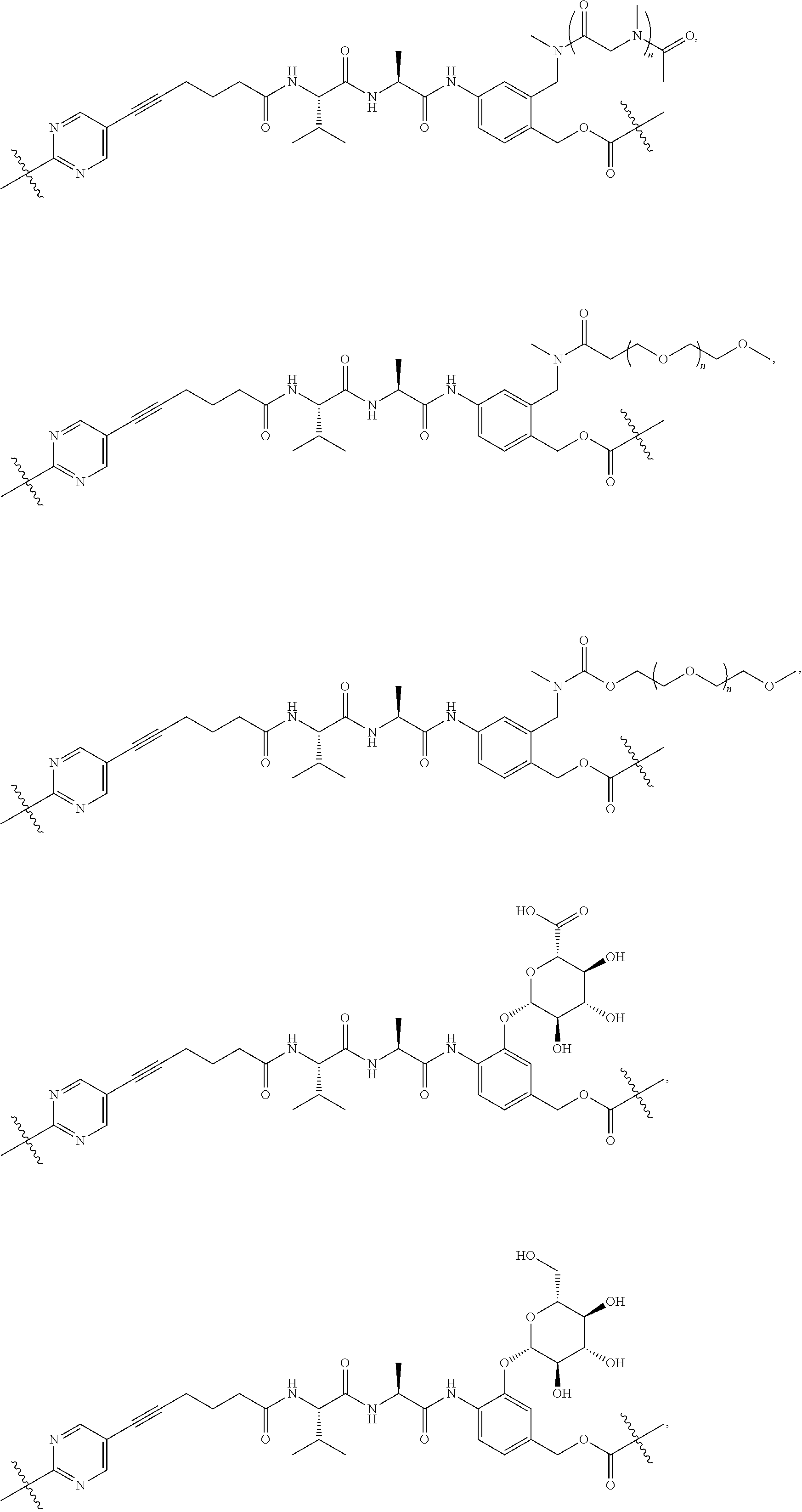

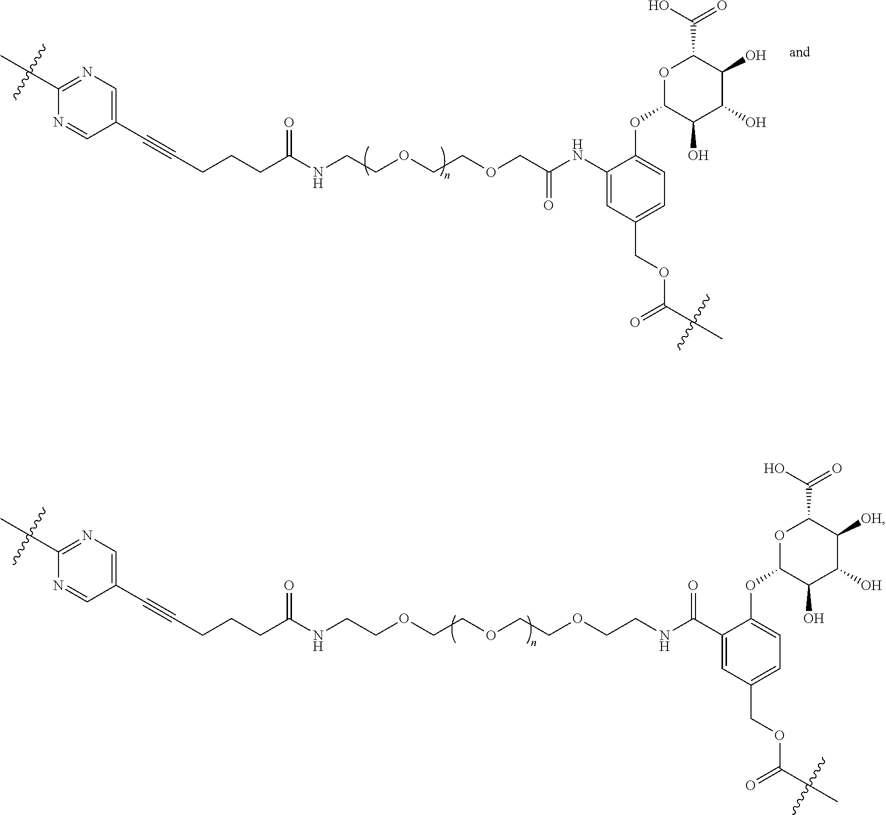

n is an integer selected from 1 to 20.

In some embodiments,

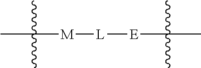

is selected from the group consisting of the following substituted or unsubstituted structural fragments:

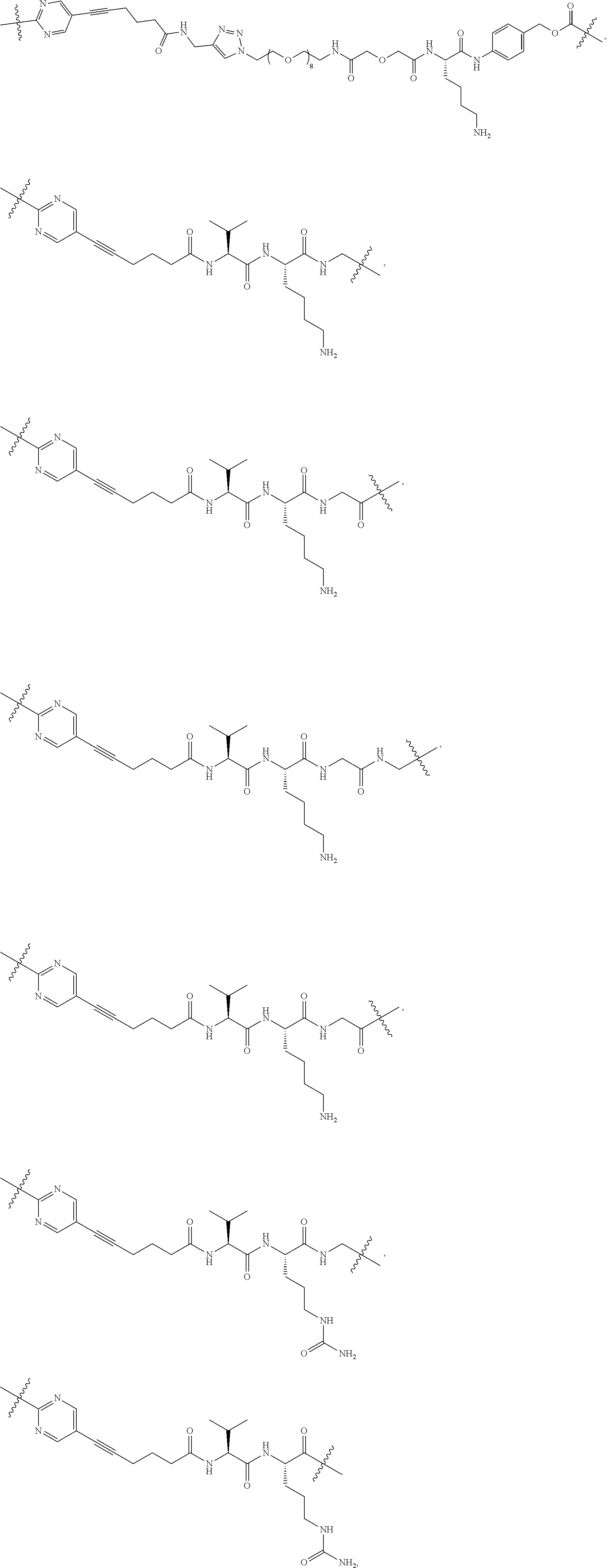

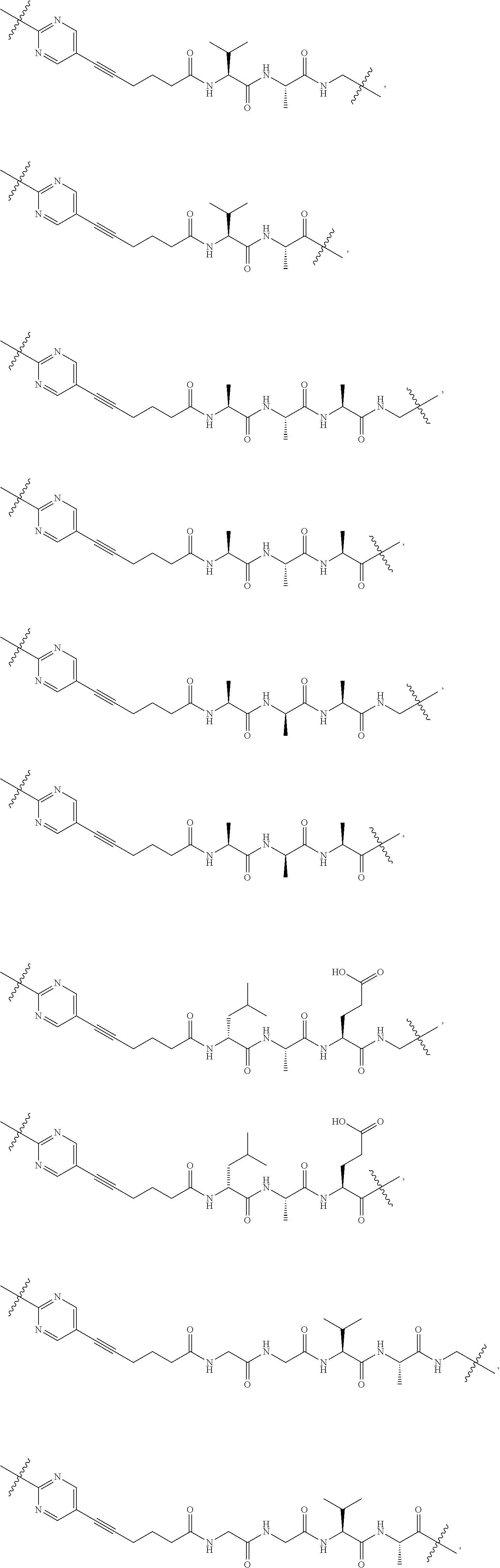

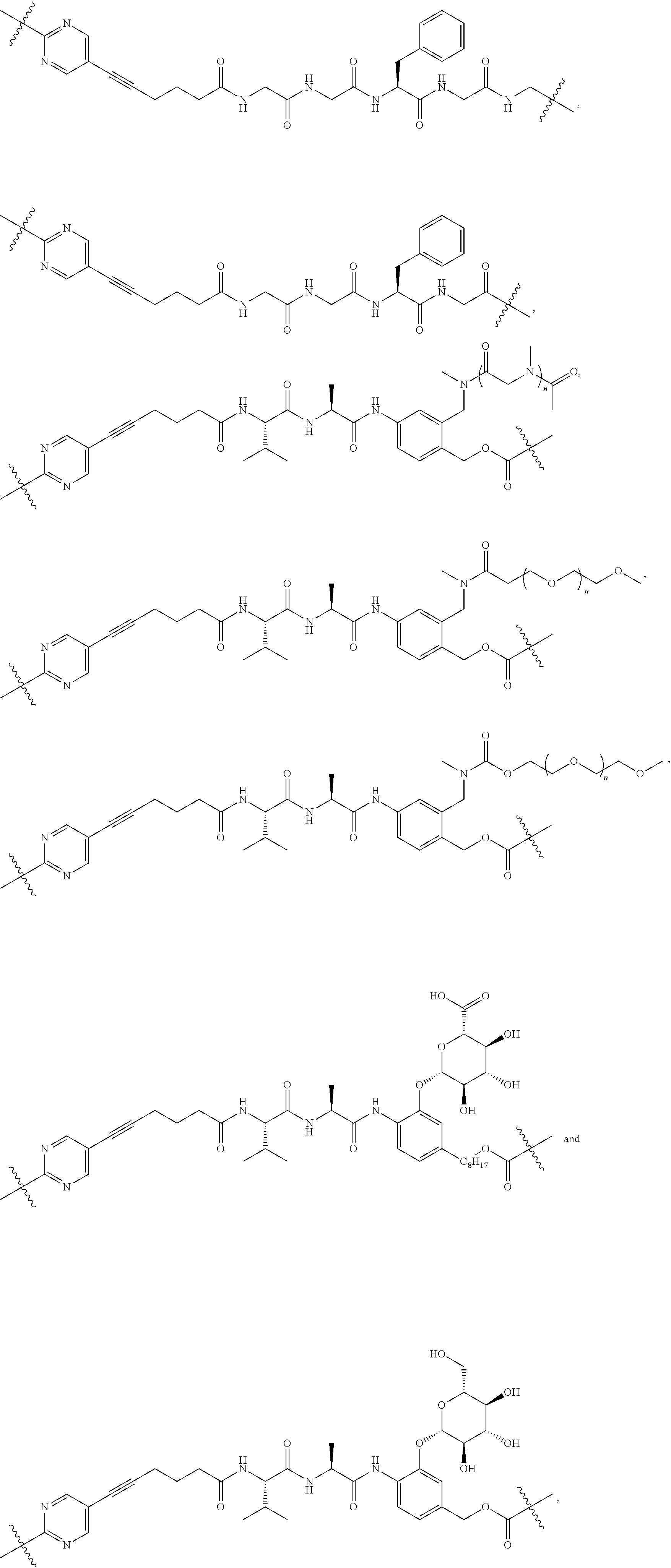

n is an integer selected from 1 to 20.

In some embodiments,

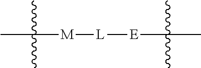

is selected from the group consisting of the following substituted or unsubstituted structural fragments:

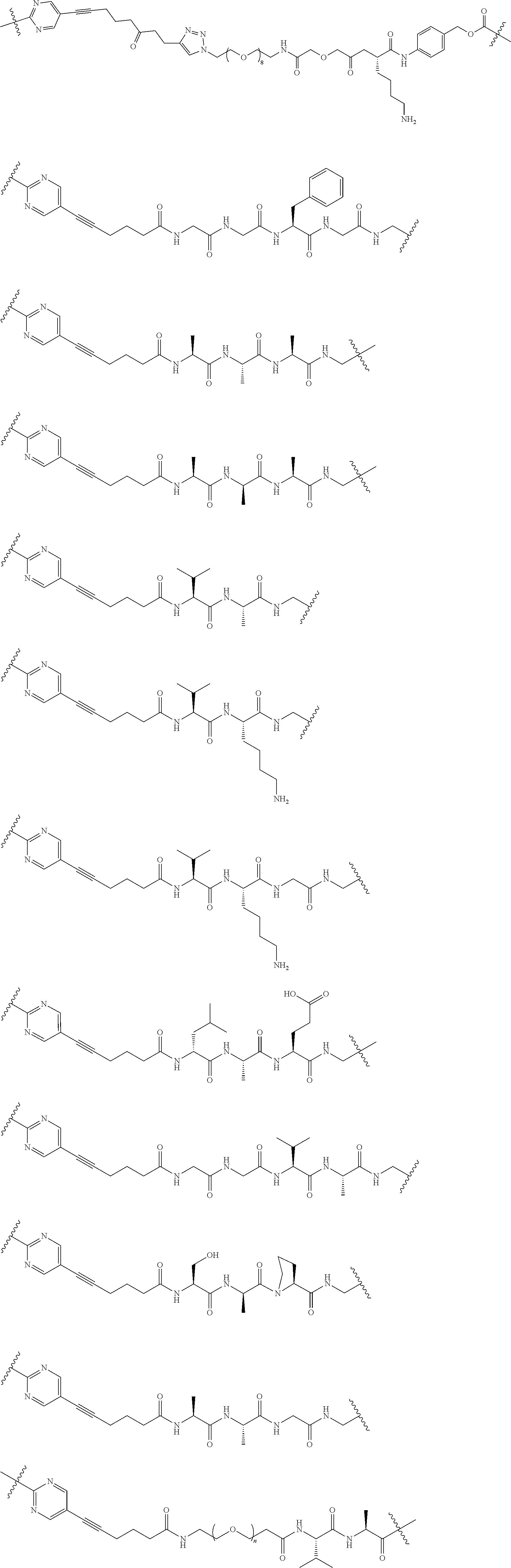

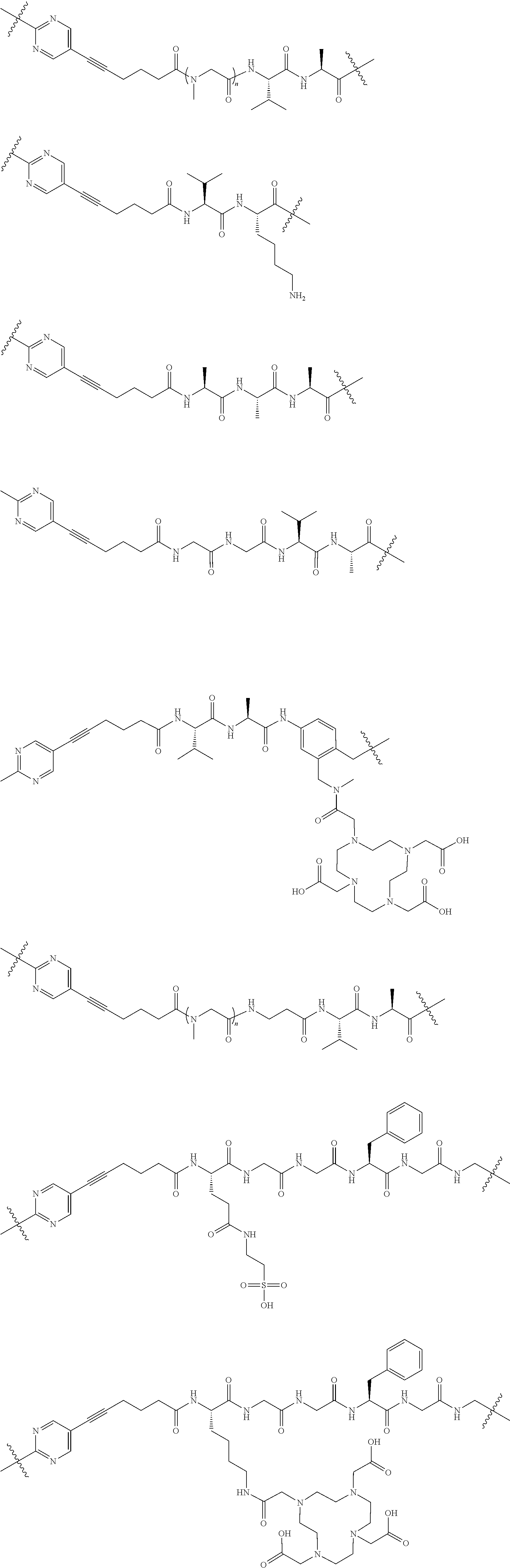

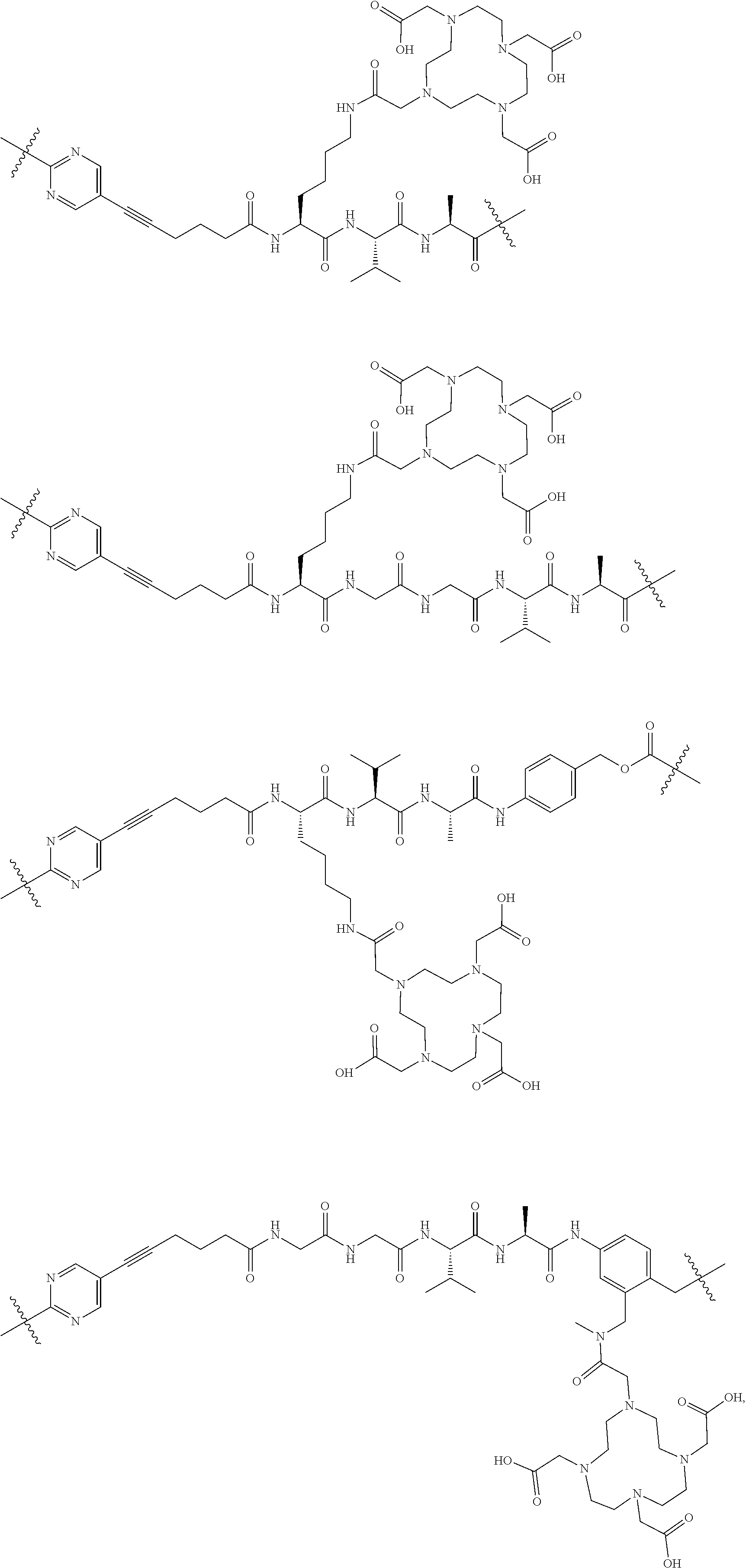

wherein, n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14 or 15, preferably or 10.

In some embodiments,

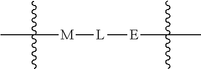

is selected from the group consisting of the following substituted or unsubstituted structural fragments:

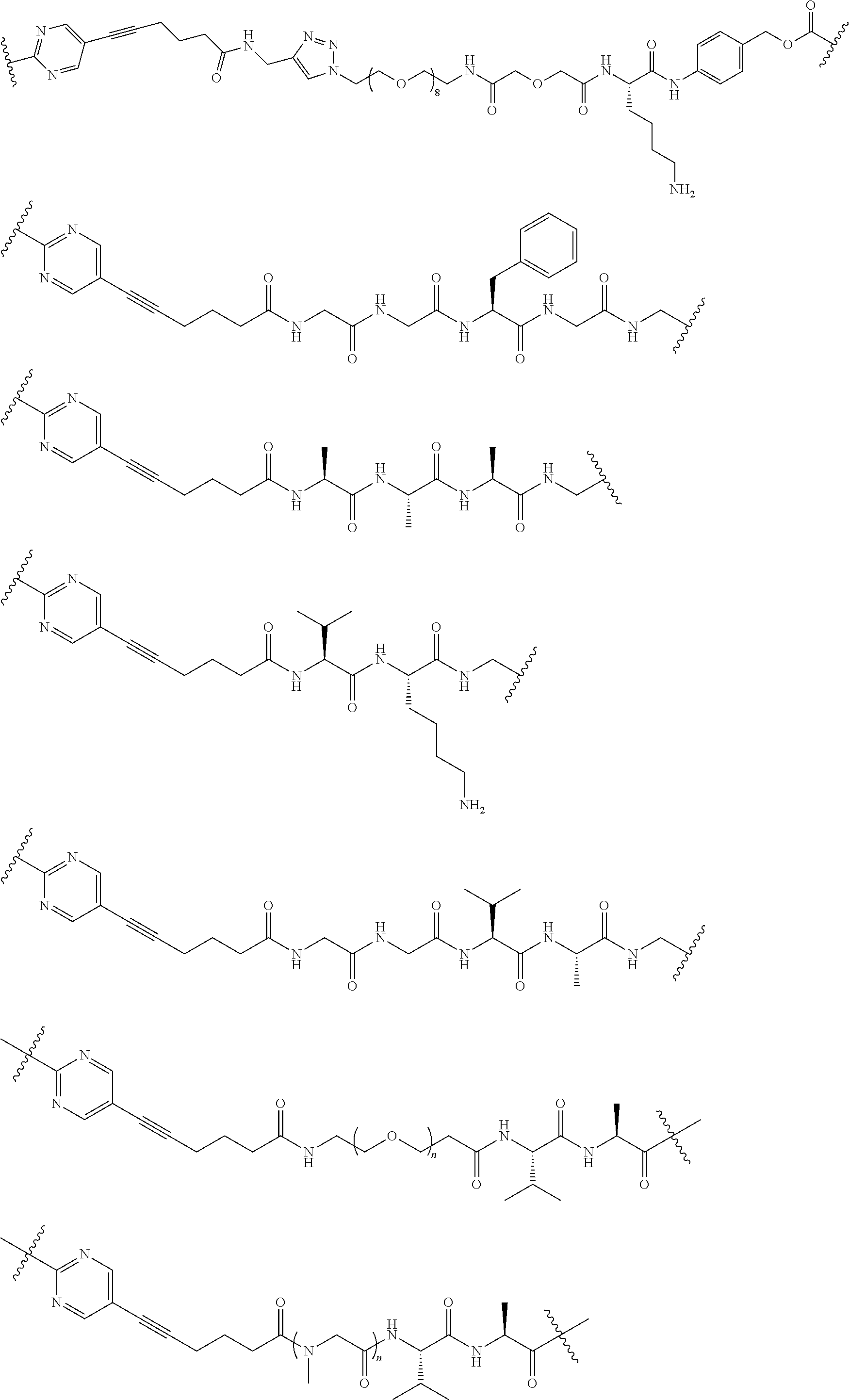

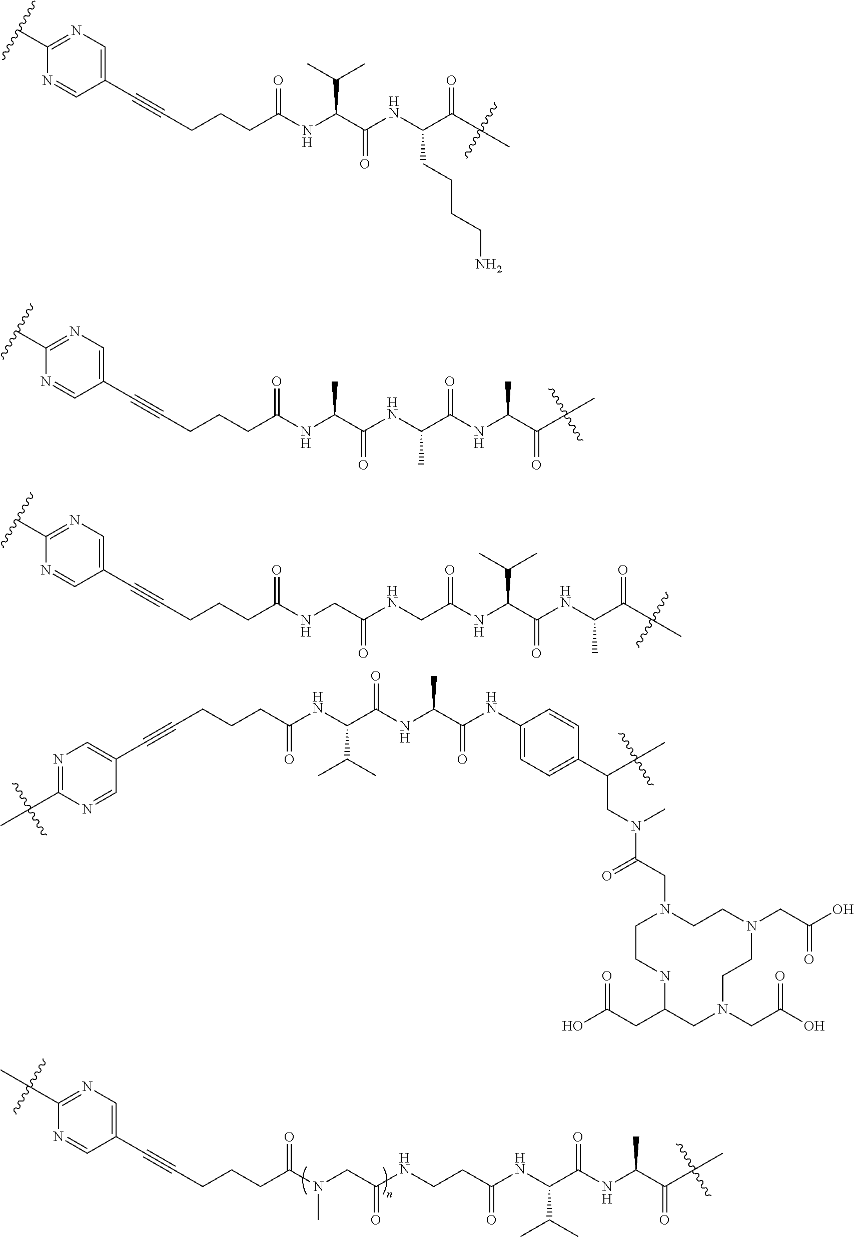

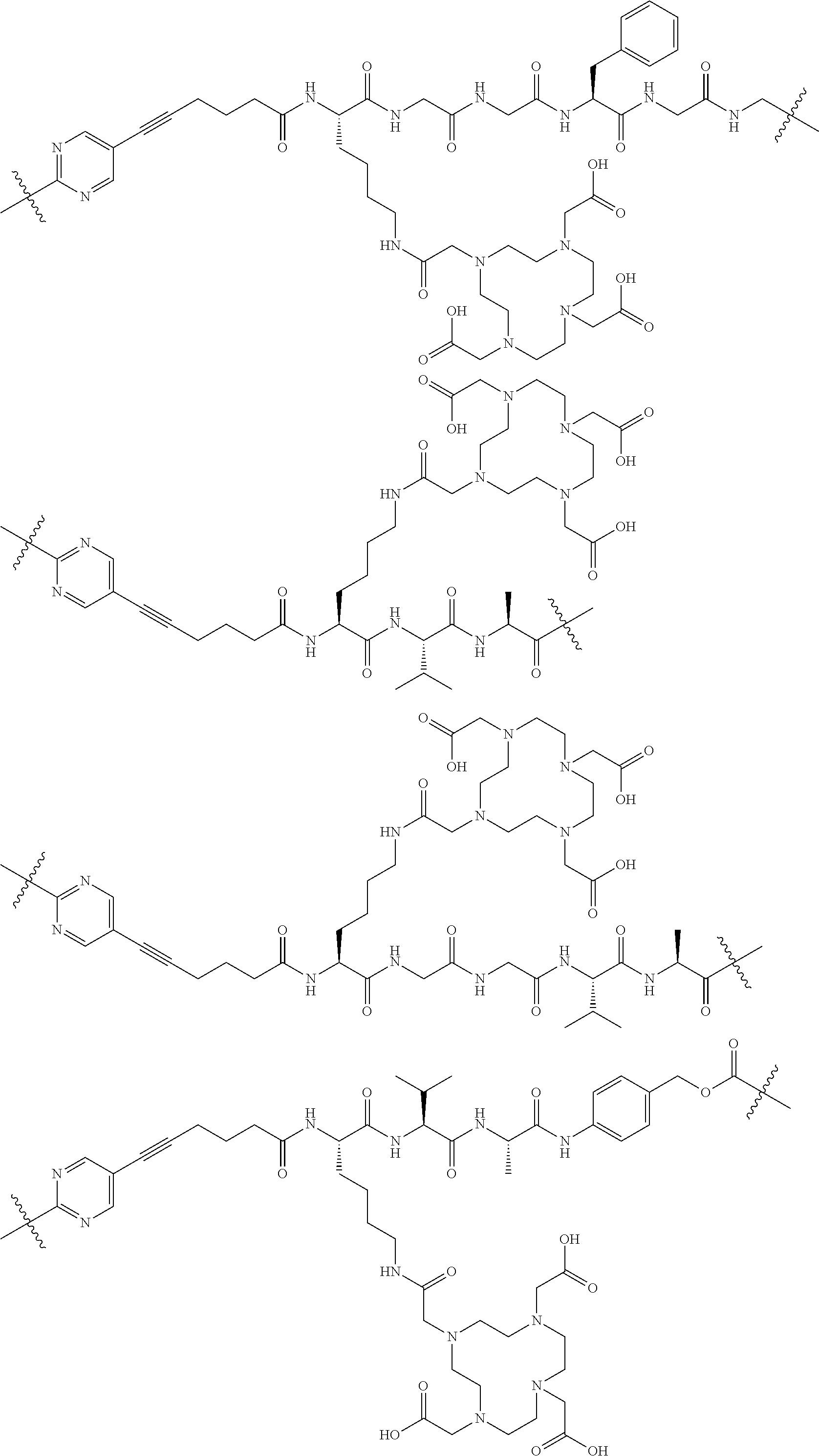

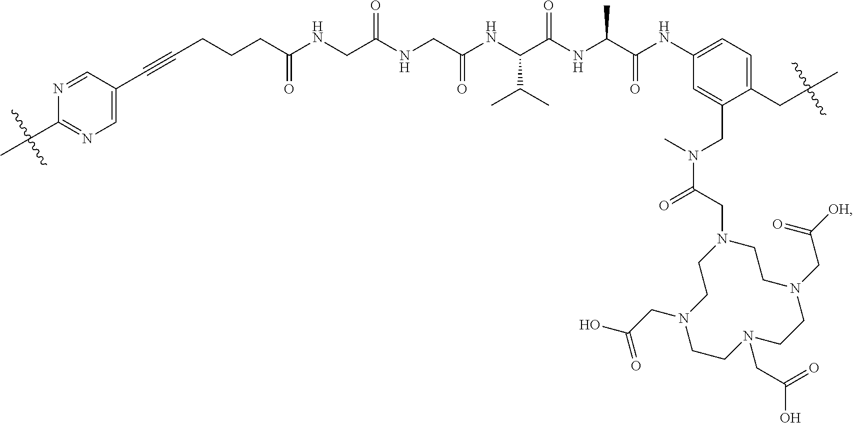

wherein n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14 or 15, preferably or 10.

In some embodiments

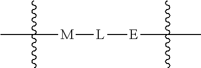

is selected from the group consisting of the following substituted or unsubstituted structural fragments:

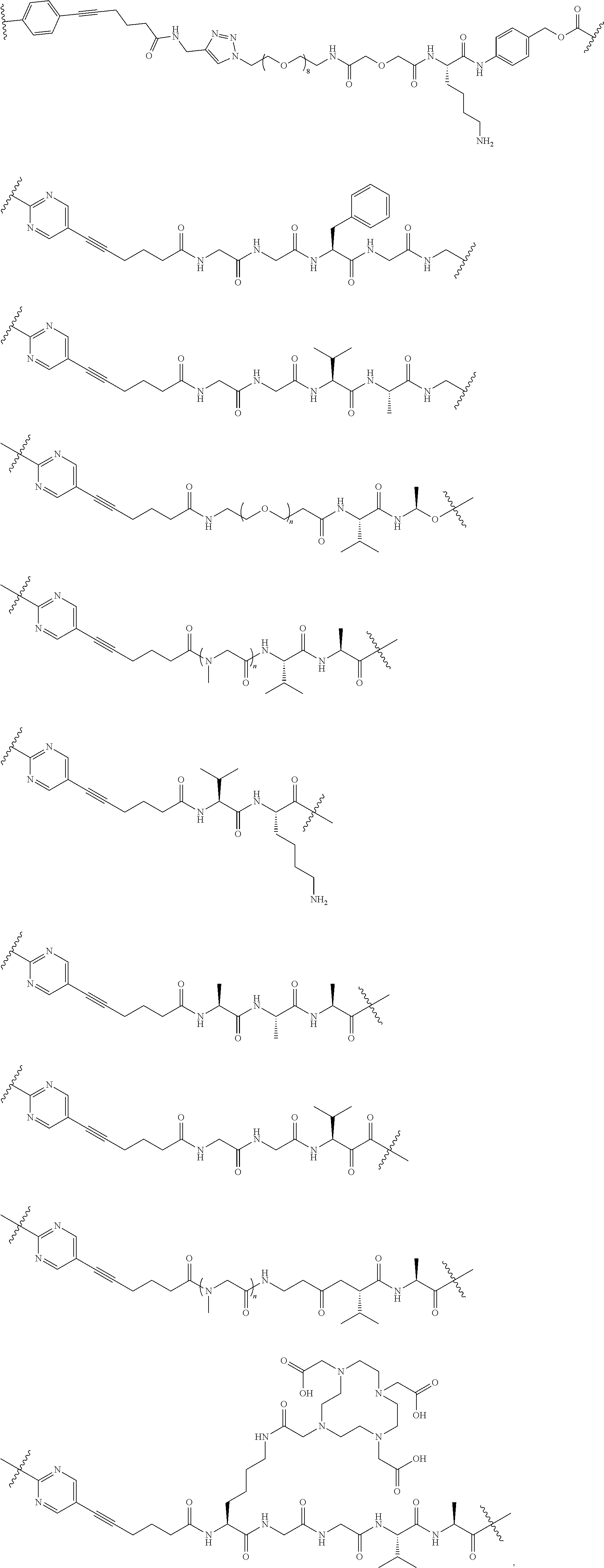

wherein n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14 or 15, preferably 8 or 10.

In some embodiments, the cytotoxic drug is selected from the group consisting of anti-tubulin agents, DNA intercalators, DNA topoisomerase inhibitors, RNA polymerase inhibitors and gene transcription inhibitors. In some embodiments, the tubulin inhibitor is an auristatin compound, a maytansine compound, or an eribulin compound. In some embodiments, the DNA intercalator is a pyrrolobenzodiazepine (PBD) compound, trabectedin, or lurbinectedin. In some embodiments, the DNA topoisomerase inhibitor is a topoisomerase I inhibitor (e.g., camptothecin, hydroxycamptothecin, 9-aminocamptothecin, SN-38, irinotecan, exatecan, topotecan, belotecan, rubitecan, diflomotecan, lurtotecan, karenitecin, gimatecan, namitecan, simmitecan, chimmitecan, silatecan, or elomotecan) or a topoisomerase II inhibitor (e.g., adriamycin, doxorubicin, PNU-159682 and analogs thereof, duocarmycin, daunorubicin, mitoxantrone, podophyllotoxin, or etoposide). In some embodiments, the RNA polymerase inhibitor is α-amanitin. In some embodiments, the gene transcription inhibitor is triptolide and pharmaceutically acceptable salts, esters, and analogs thereof.

In some embodiments, the cytotoxic drug is a topoisomerase I inhibitor (e.g., camptothecin, hydroxycamptothecin, 9-aminocamptothecin, SN-38, irinotecan, exatecan, topotecan, belotecan, rubitecan, diflomotecan, lurtotecan, karenitecin, gimatecan, namitecan, simmitecan, chimmitecan, silatecan, or elomotecan).

The cytotoxic drug disclosed in the present application typically comprises a variety of functional groups, such as hydroxyl (—OH), carboxyl (—COOH), primary amino (—NH₂), secondary amino (—NR₁H), tertiary amino (—NR₂R₃), wherein R₁, R₂, R₃ here represent only non-hydrogen substituents on N, or sulfhydryl group (—SH), and these functional groups can react with a suitable functional group in the rest of the conjugate to achieve connection.

In some embodiments, the cytotoxic drug is connected through —OH, primary amino, secondary amino or tertiary amino, or —SH thereon to E in the antibody-drug conjugate. In some embodiments, D is a monovalent structure obtained by losing one H from —OH, —NH₂ or secondary amino on the cytotoxic drug.

In some embodiments, the cytotoxic drug is selected from the group consisting of the following compounds or isotopically labeled compounds thereof:

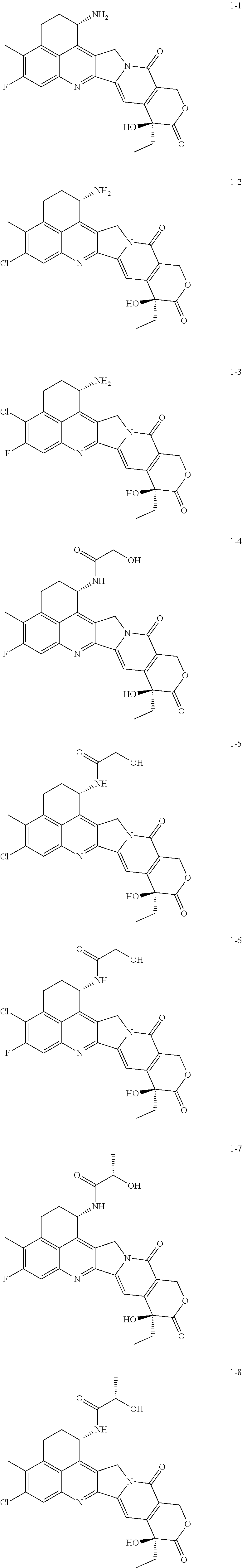

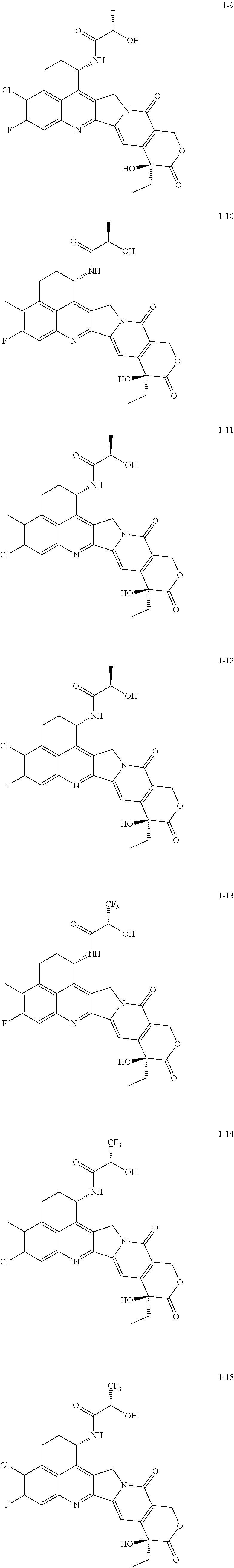

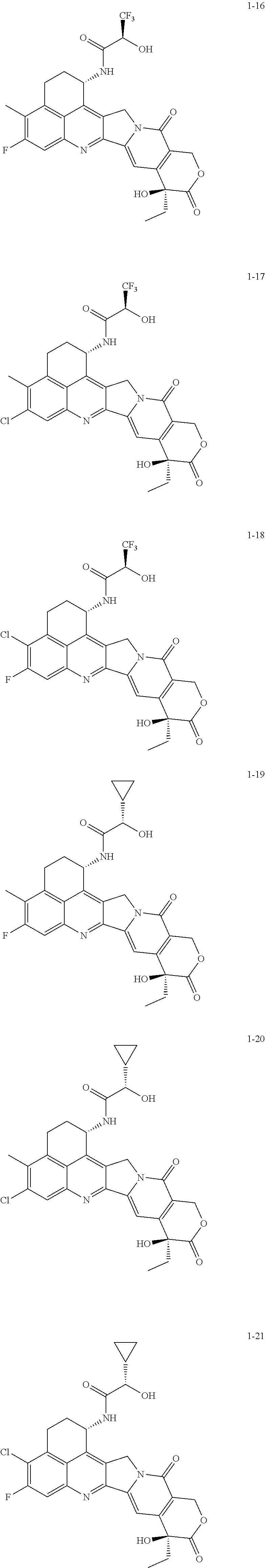

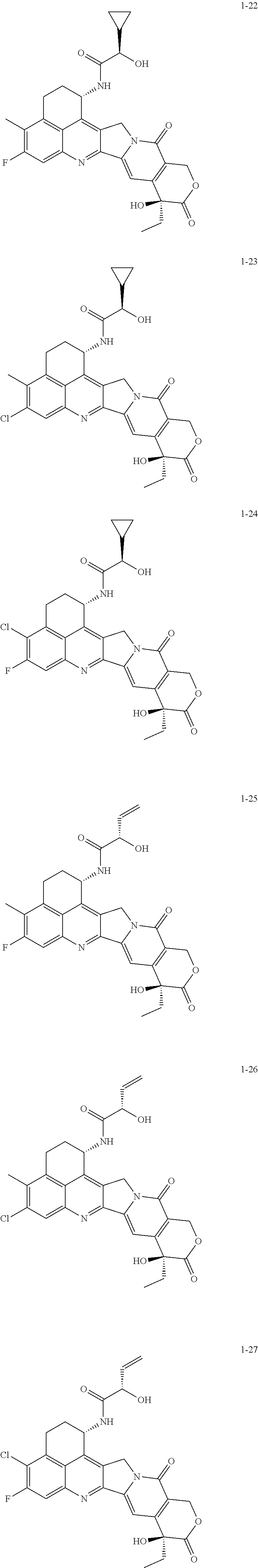

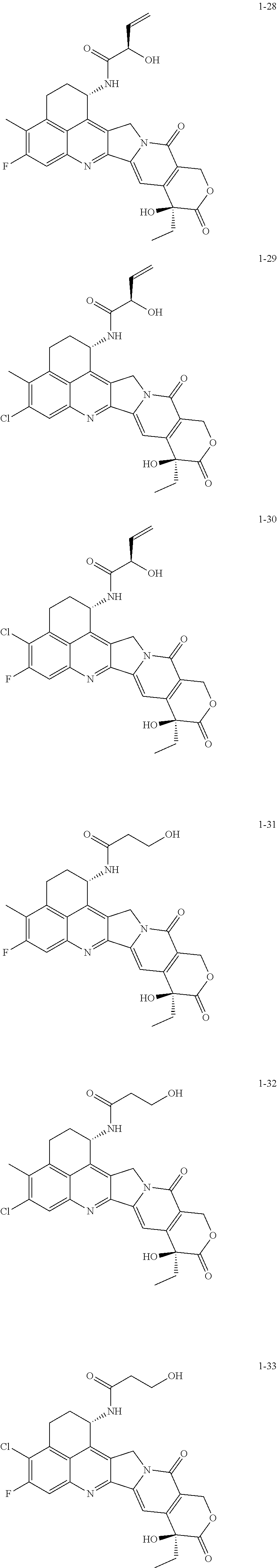

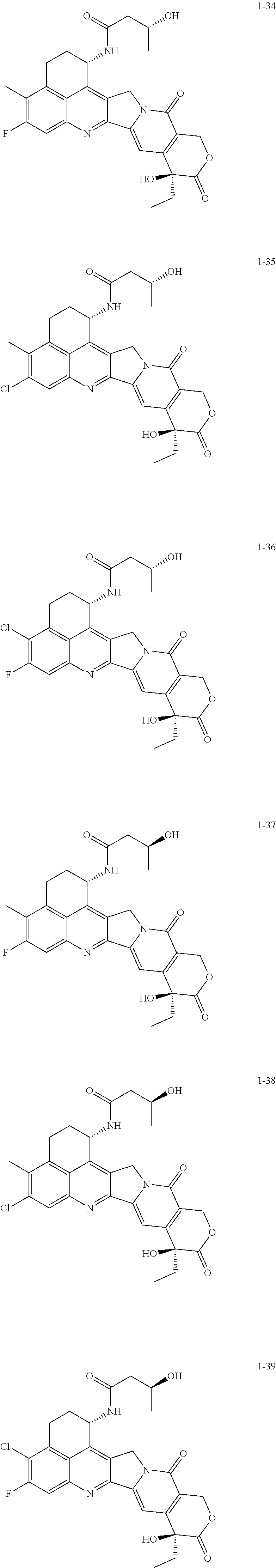

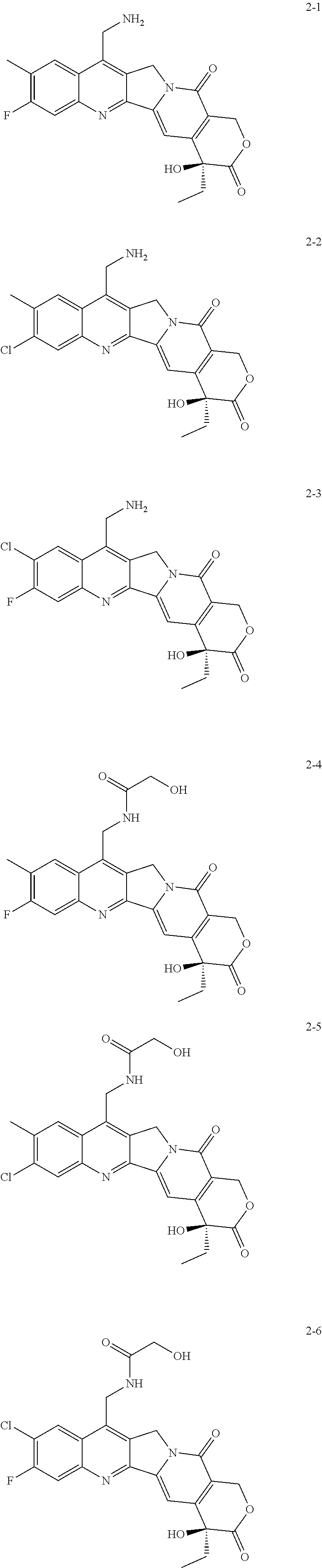 [SMILES: CC[C@@]1(O)C(=O)OCc2c1cc1n(c2=O)Cc2c-1nc1cc(F)c(C)cc1c2CN ; CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(F)c(C)cc1c2CNC(=O)CO ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO]

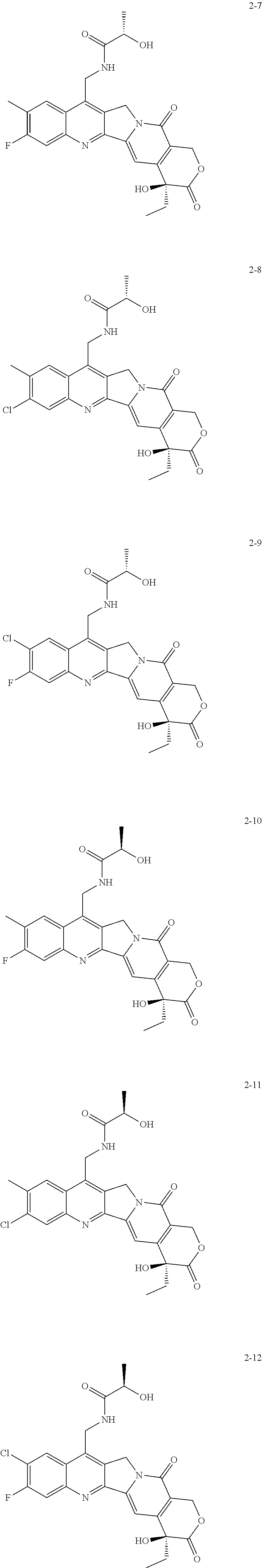 [SMILES: CC[C@@]1(O)C(=O)OCc2c1cc1n(c2=O)Cc2c-1nc1cc(F)c(C)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(F)c(C)cc1c2CNC(=O)[C@@H](C)O ; CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O]

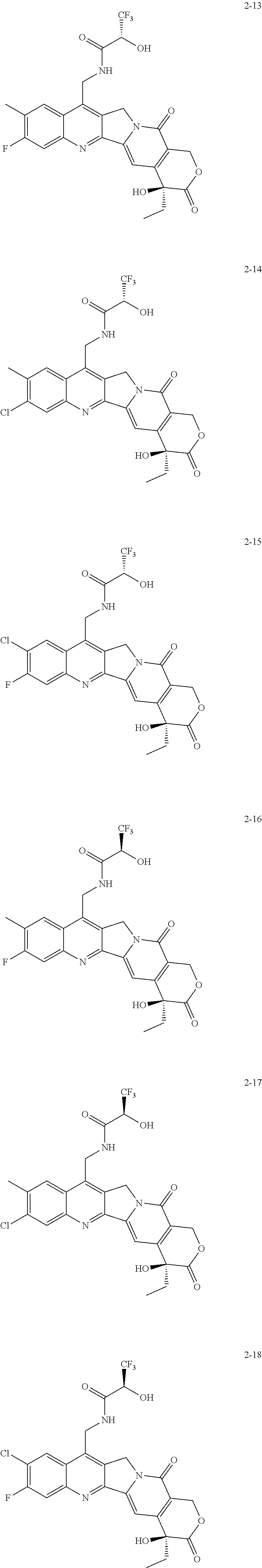 [SMILES: CC[C@@]1(O)C(=O)OCc2c1cc1n(c2=O)Cc2c-1nc1cc(F)c(C)cc1c2CNC(=O)[C@@H](O)C(F)(F)F ; CC[C@@]1(O)C(=O)OCc2c1cc1n(c2=O)Cc2c-1nc1cc(Cl)c(C)cc1c2CNC(=O)[C@@H](O)C(F)(F)F ; CC[C@@]1(O)C(=O)OCc2c1cc1n(c2=O)Cc2c-1nc1cc(F)c(Cl)cc1c2CNC(=O)[C@@H](O)C(F)(F)F ; CC[C@@]1(O)C(=O)OCc2c1cc1n(c2=O)Cc2c-1nc1cc(F)c(C)cc1c2CNC(=O)[C@H](O)C(F)(F)F ; CC[C@@]1(O)C(=O)OCc2c1cc1n(c2=O)Cc2c-1nc1cc(Cl)c(C)cc1c2CNC(=O)[C@H](O)C(F)(F)F ; CC[C@@]1(O)C(=O)OCc2c1cc1n(c2=O)Cc2c-1nc1cc(F)c(Cl)cc1c2CNC(=O)[C@H](O)C(F)(F)F]

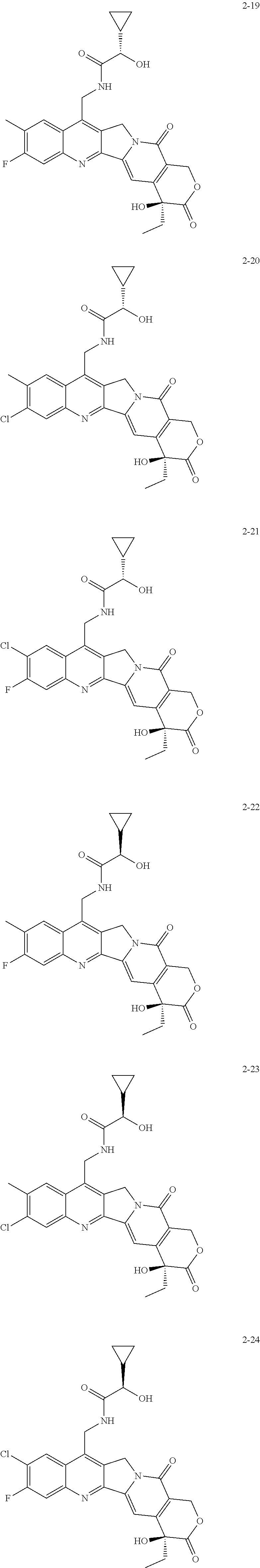 [SMILES: CC[C@@]1(O)C(=O)OCc2c1cc1n(c2=O)Cc2c-1nc1cc(F)c(C)cc1c2CNC(=O)[C@@H](O)C1CC1 ; CC[C@@]1(O)C(=O)OCc2c1cc1n(c2=O)Cc2c-1nc1cc(Cl)c(C)cc1c2CNC(=O)[C@@H](O)C1CC1 ; CC[C@@]1(O)C(=O)OCc2c1cc1n(c2=O)Cc2c-1nc1cc(F)c(Cl)cc1c2CNC(=O)[C@@H](O)C1CC1 ; CC[C@@]1(O)C(=O)OCc2c1cc1n(c2=O)Cc2c-1nc1cc(F)c(C)cc1c2CNC(=O)[C@H](O)C1CC1 ; CC[C@@]1(O)C(=O)OCc2c1cc1n(c2=O)Cc2c-1nc1cc(Cl)c(C)cc1c2CNC(=O)[C@H](O)C1CC1 ; CC[C@@]1(O)C(=O)OCc2c1cc1n(c2=O)Cc2c-1nc1cc(F)c(Cl)cc1c2CNC(=O)[C@H](O)C1CC1]

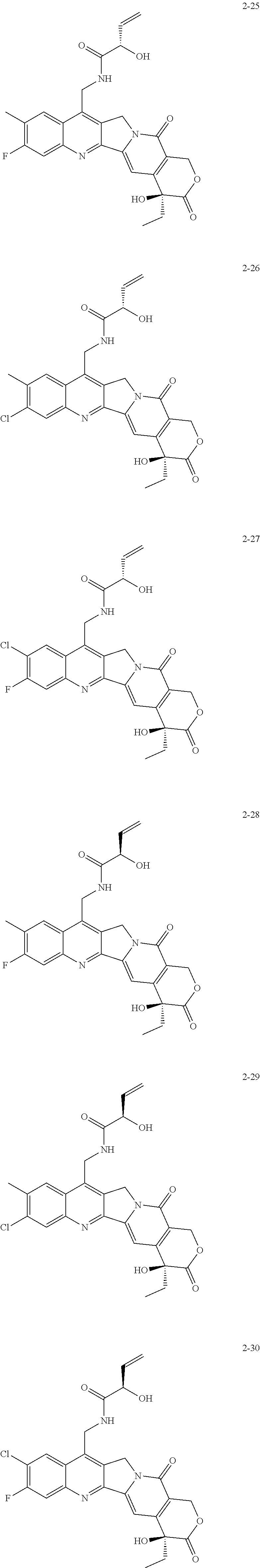 [SMILES: C=C[C@H](O)C(=O)NCc1c2c(nc3cc(F)c(C)cc13)-c1cc3c(c(=O)n1C2)COC(=O)[C@]3(O)CC ; C=C[C@H](O)C(=O)NCc1c2c(nc3cc(Cl)c(C)cc13)-c1cc3c(c(=O)n1C2)COC(=O)[C@]3(O)CC ; C=C[C@H](O)C(=O)NCc1c2c(nc3cc(F)c(Cl)cc13)-c1cc3c(c(=O)n1C2)COC(=O)[C@]3(O)CC ; C=C[C@@H](O)C(=O)NCc1c2c(nc3cc(F)c(C)cc13)-c1cc3c(c(=O)n1C2)COC(=O)[C@]3(O)CC ; C=C[C@@H](O)C(=O)NCc1c2c(nc3cc(Cl)c(C)cc13)-c1cc3c(c(=O)n1C2)COC(=O)[C@]3(O)CC ; C=C[C@@H](O)C(=O)NCc1c2c(nc3cc(F)c(Cl)cc13)-c1cc3c(c(=O)n1C2)COC(=O)[C@]3(O)CC]

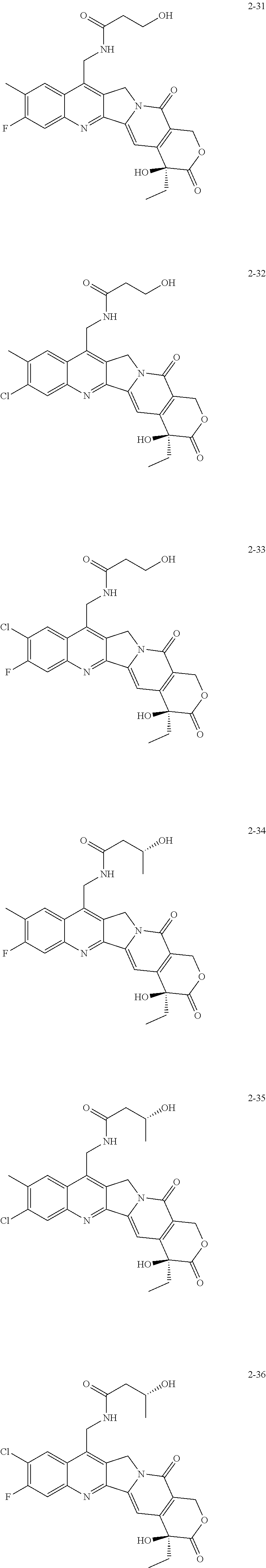 [SMILES: CC[C@@]1(O)C(=O)OCc2c1cc1n(c2=O)Cc2c-1nc1cc(F)c(C)cc1c2CNC(=O)CCO ; CC[C@@]1(O)C(=O)OCc2c1cc1n(c2=O)Cc2c-1nc1cc(Cl)c(C)cc1c2CNC(=O)CCO ; CC[C@@]1(O)C(=O)OCc2c1cc1n(c2=O)Cc2c-1nc1cc(F)c(Cl)cc1c2CNC(=O)CCO ; CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O]

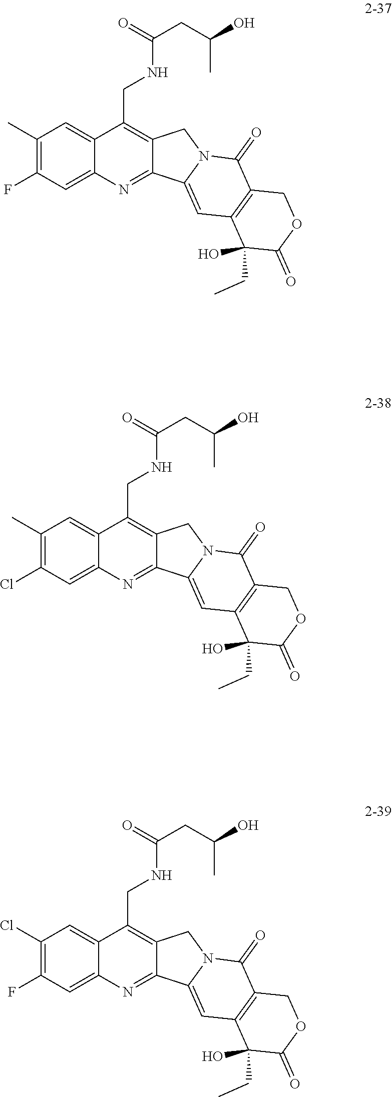 [SMILES: CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O]

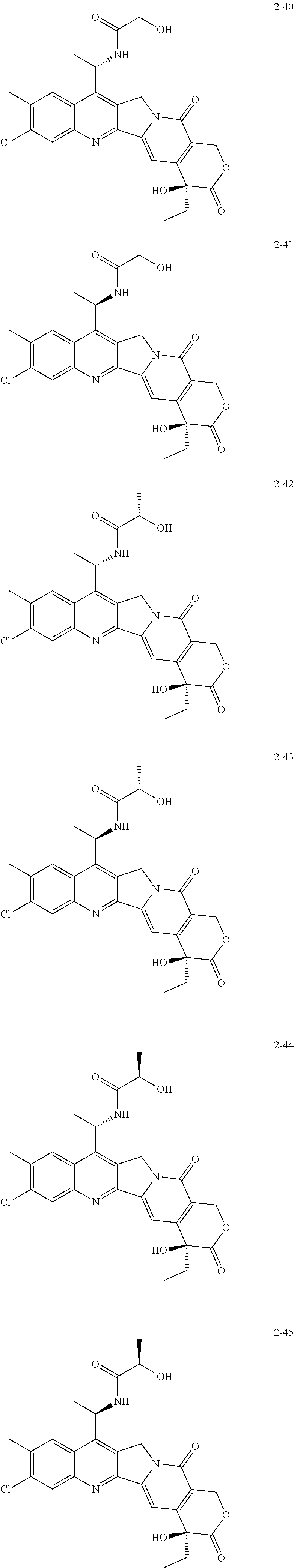 [SMILES: CC[C@@]1(O)C(=O)OCc2c1cc1n(c2=O)Cc2c-1nc1cc(Cl)c(C)cc1c2[C@H](C)NC(=O)CO ; CC[C@@]1(O)C(=O)OCc2c1cc1n(c2=O)Cc2c-1nc1cc(Cl)c(C)cc1c2[C@@H](C)NC(=O)CO ; CC[C@@]1(O)C(=O)OCc2c1cc1n(c2=O)Cc2c-1nc1cc(Cl)c(C)cc1c2[C@H](C)NC(=O)[C@H](C)O ; CC[C@@]1(O)C(=O)OCc2c1cc1n(c2=O)Cc2c-1nc1cc(Cl)c(C)cc1c2[C@@H](C)NC(=O)[C@H](C)O ; CC[C@@]1(O)C(=O)OCc2c1cc1n(c2=O)Cc2c-1nc1cc(Cl)c(C)cc1c2[C@H](C)NC(=O)[C@@H](C)O ; CC[C@@]1(O)C(=O)OCc2c1cc1n(c2=O)Cc2c-1nc1cc(Cl)c(C)cc1c2[C@@H](C)NC(=O)[C@@H](C)O]

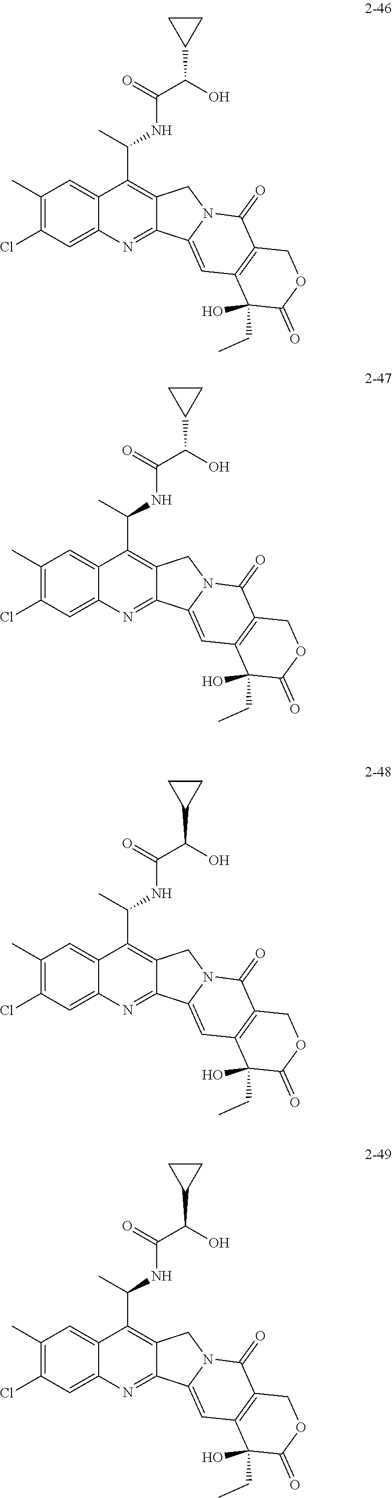

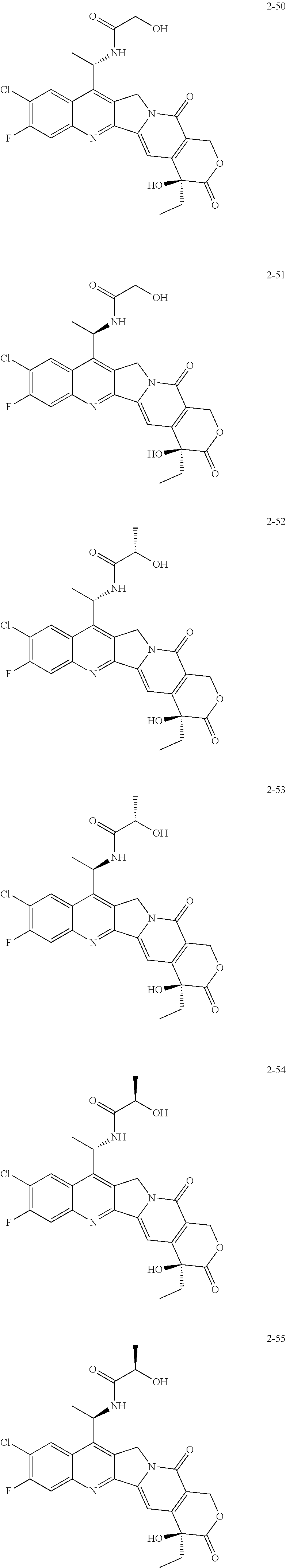 [SMILES: CC[C@@]1(O)C(=O)OCc2c1cc1n(c2=O)Cc2c-1nc1cc(F)c(Cl)cc1c2[C@H](C)NC(=O)CO ; CC[C@@]1(O)C(=O)OCc2c1cc1n(c2=O)Cc2c-1nc1cc(F)c(Cl)cc1c2[C@@H](C)NC(=O)CO ; CC[C@@]1(O)C(=O)OCc2c1cc1n(c2=O)Cc2c-1nc1cc(F)c(Cl)cc1c2[C@H](C)NC(=O)[C@H](C)O ; CC[C@@]1(O)C(=O)OCc2c1cc1n(c2=O)Cc2c-1nc1cc(F)c(Cl)cc1c2[C@@H](C)NC(=O)[C@H](C)O ; CC[C@@]1(O)C(=O)OCc2c1cc1n(c2=O)Cc2c-1nc1cc(F)c(Cl)cc1c2[C@H](C)NC(=O)[C@@H](C)O ; CC[C@@]1(O)C(=O)OCc2c1cc1n(c2=O)Cc2c-1nc1cc(F)c(Cl)cc1c2[C@@H](C)NC(=O)[C@@H](C)O]

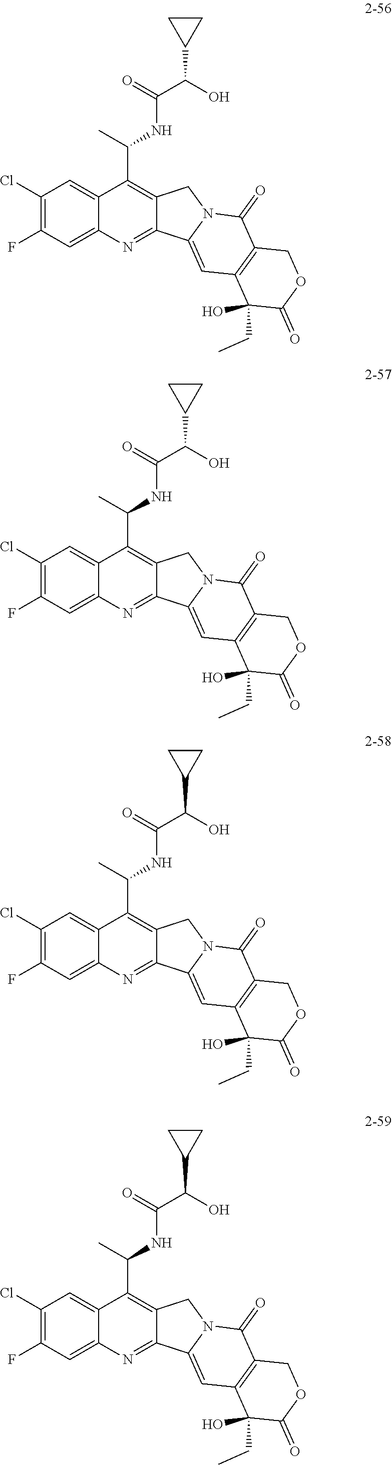

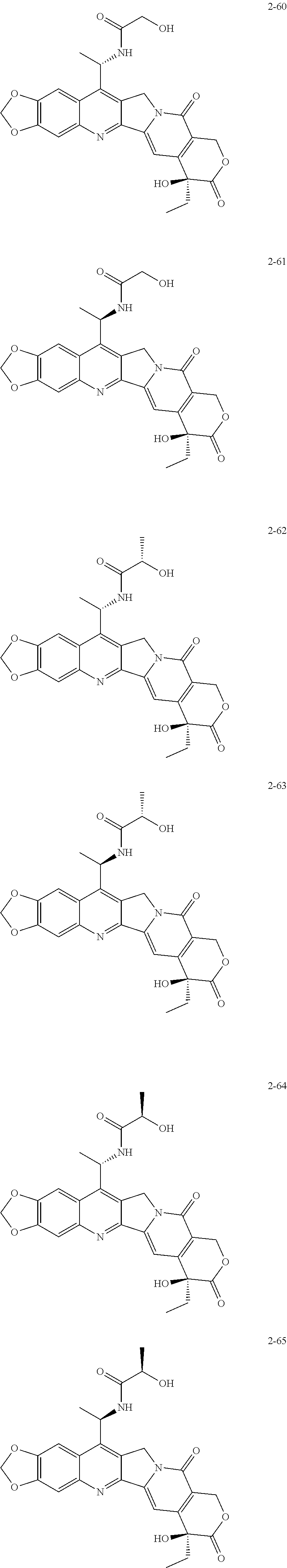 [SMILES: CC[C@@]1(O)C(=O)OCc2c1cc1n(c2=O)Cc2c-1nc1cc3c(cc1c2[C@H](C)NC(=O)CO)OCO3 ; CC[C@@]1(O)C(=O)OCc2c1cc1n(c2=O)Cc2c-1nc1cc3c(cc1c2[C@@H](C)NC(=O)CO)OCO3 ; CC[C@@]1(O)C(=O)OCc2c1cc1n(c2=O)Cc2c-1nc1cc3c(cc1c2[C@H](C)NC(=O)[C@H](C)O)OCO3 ; CC[C@@]1(O)C(=O)OCc2c1cc1n(c2=O)Cc2c-1nc1cc3c(cc1c2[C@@H](C)NC(=O)[C@H](C)O)OCO3 ; CC[C@@]1(O)C(=O)OCc2c1cc1n(c2=O)Cc2c-1nc1cc3c(cc1c2[C@H](C)NC(=O)[C@@H](C)O)OCO3 ; CC[C@@]1(O)C(=O)OCc2c1cc1n(c2=O)Cc2c-1nc1cc3c(cc1c2[C@@H](C)NC(=O)[C@@H](C)O)OCO3]

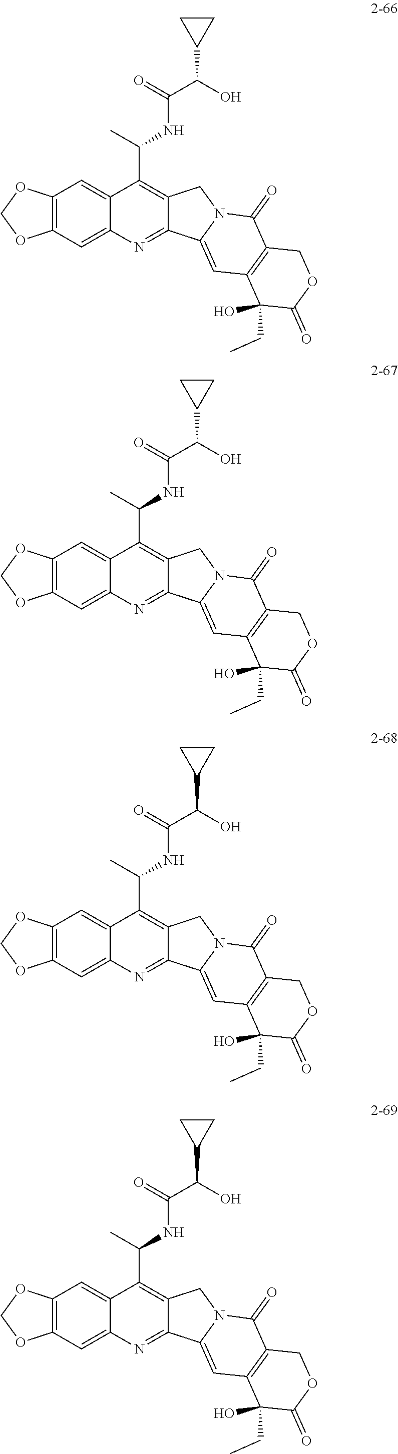

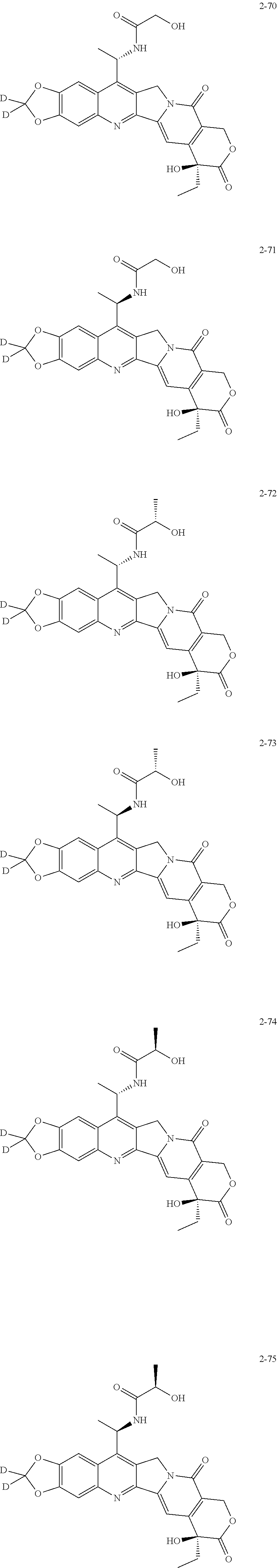

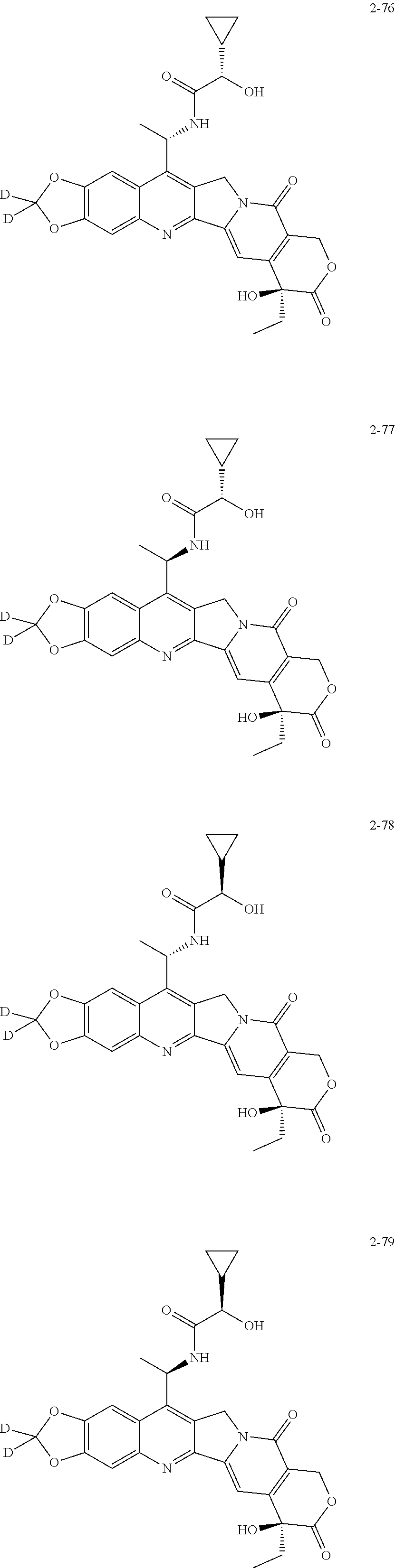 [SMILES: [2H]C1([2H])Oc2cc3nc4c(c([C@H](C)NC(=O)[C@@H](O)C5CC5)c3cc2O1)Cn1c-4cc2c(c1=O)COC(=O)[C@]2(O)CC ; [2H]C1([2H])Oc2cc3nc4c(c([C@@H](C)NC(=O)[C@@H](O)C5CC5)c3cc2O1)Cn1c-4cc2c(c1=O)COC(=O)[C@]2(O)CC ; [2H]C1([2H])Oc2cc3nc4c(c([C@H](C)NC(=O)[C@H](O)C5CC5)c3cc2O1)Cn1c-4cc2c(c1=O)COC(=O)[C@]2(O)CC ; [2H]C1([2H])Oc2cc3nc4c(c([C@@H](C)NC(=O)[C@H](O)C5CC5)c3cc2O1)Cn1c-4cc2c(c1=O)COC(=O)[C@]2(O)CC]

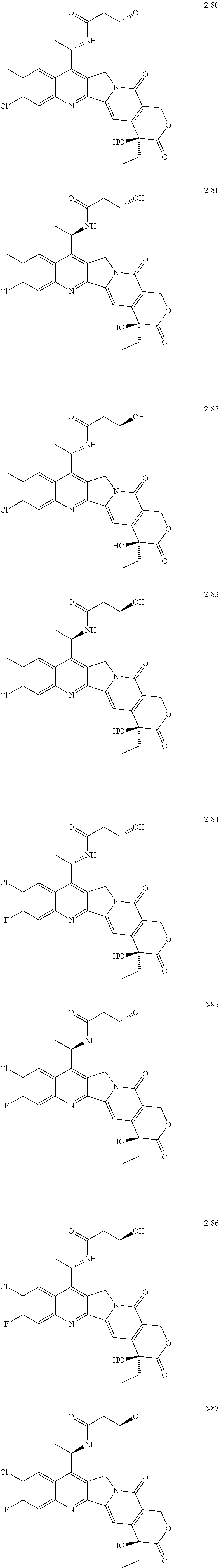

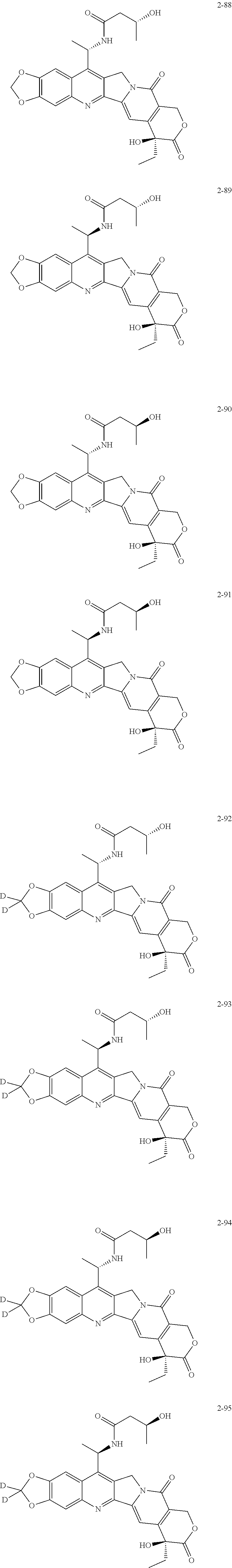

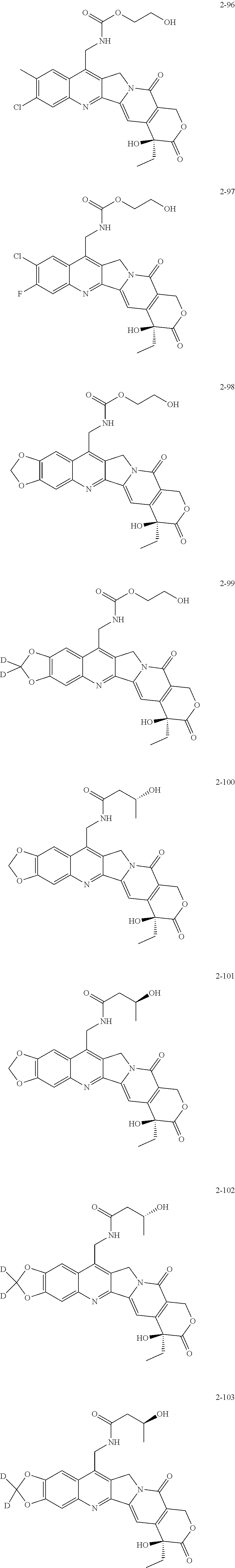

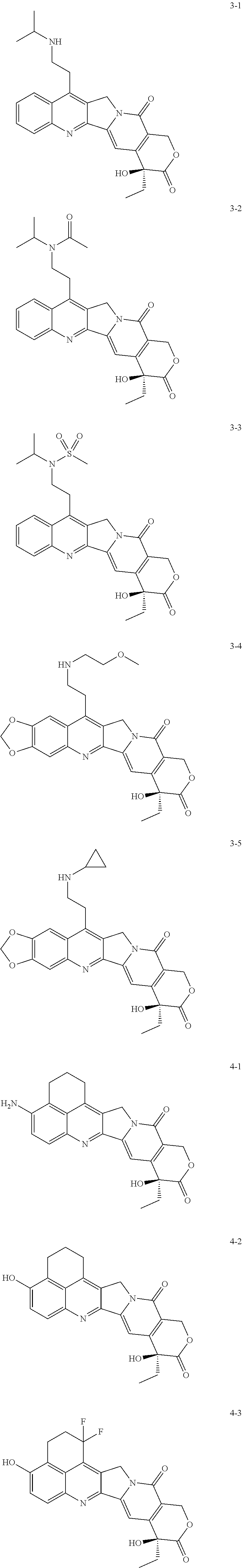

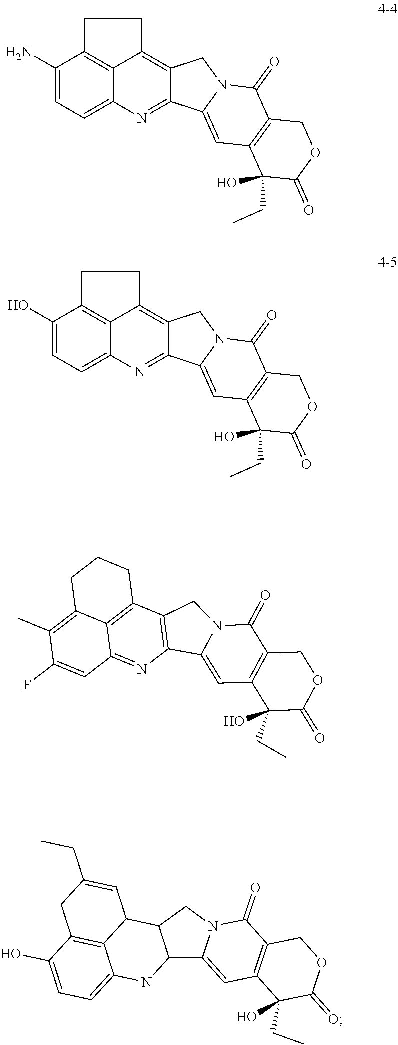 [SMILES: CC[C@@]1(O)C(=O)OCc2c1cc1n(c2=O)Cc2c-1nc1ccc(N)c3c1c2CC3 ; CC[C@@]1(O)C(=O)OCc2c1cc1n(c2=O)Cc2c-1nc1ccc(O)c3c1c2CC3 ; CC[C@@]1(O)C(=O)OCc2c1cc1n(c2=O)Cc2c-1nc1cc(F)c(C)c3c1c2CCC3 ; CCC1=CC2c3c(ccc(O)c3C1)NC1c3cc4c(c(=O)n3CC21)COC(=O)[C@]4(O)CC]

preferably, the cytotoxic drug is selected from the group consisting of the following compounds or isotopically labeled compounds thereof:

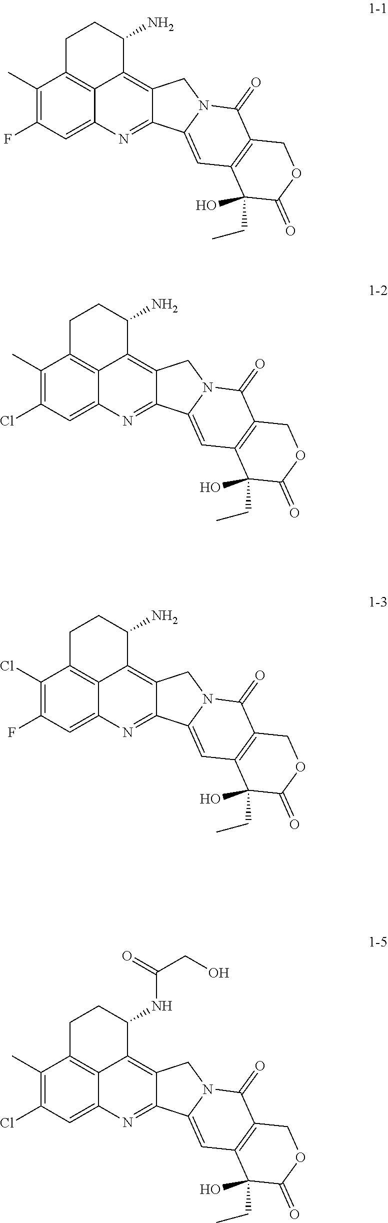 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3]

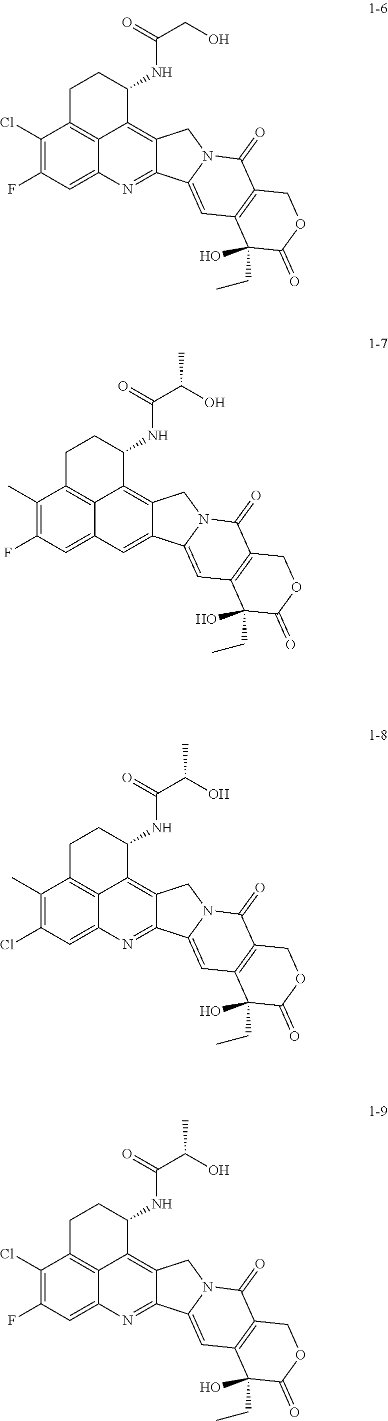 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1cc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3]

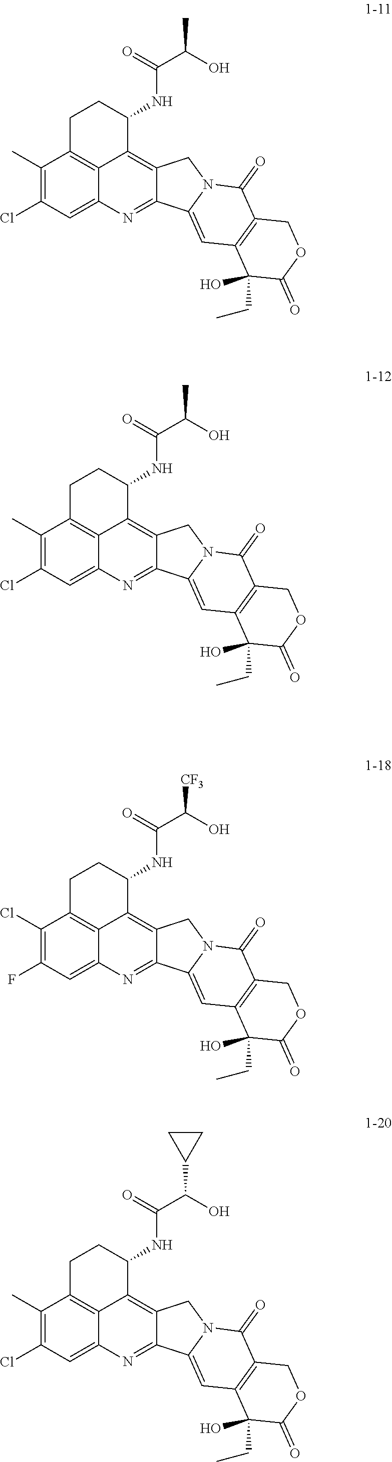 [SMILES: CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(Cl)c(C)c3c1c2[C@@H](NC(=O)[C@@H](O)C1CC1)CC3]

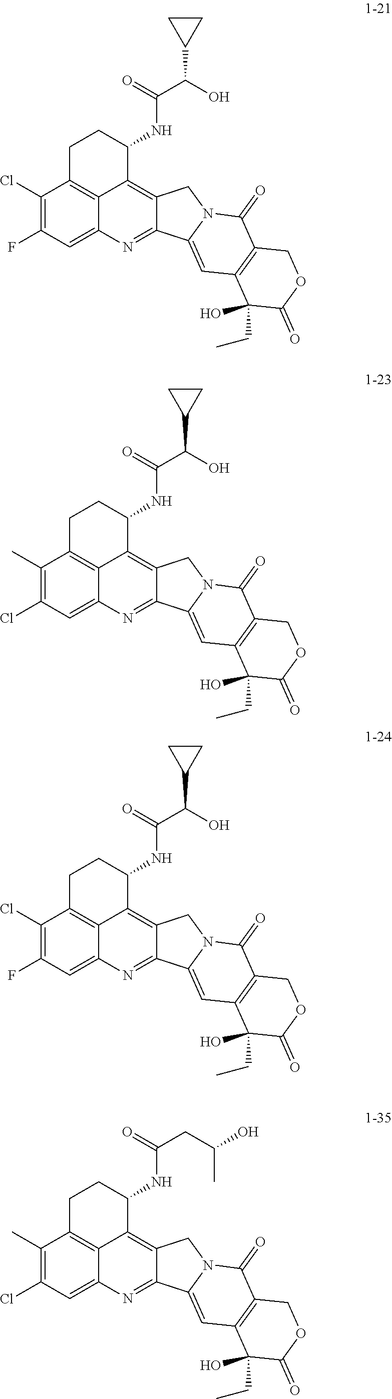

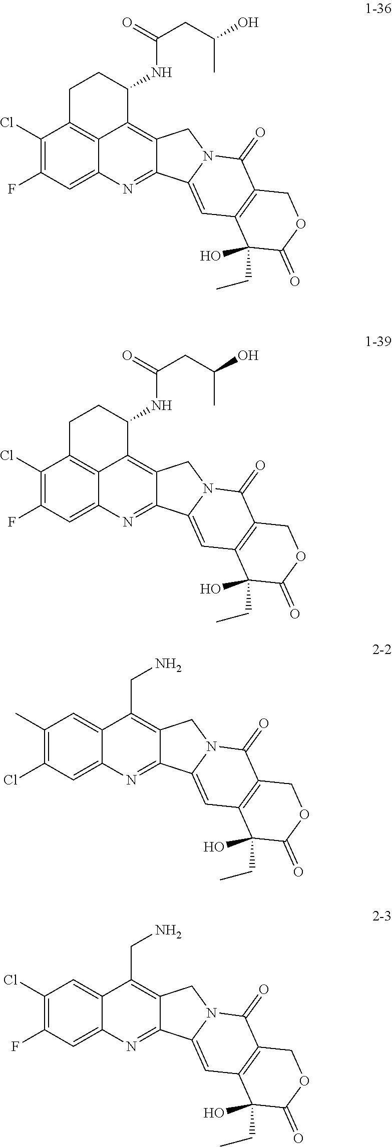 [SMILES: CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN]

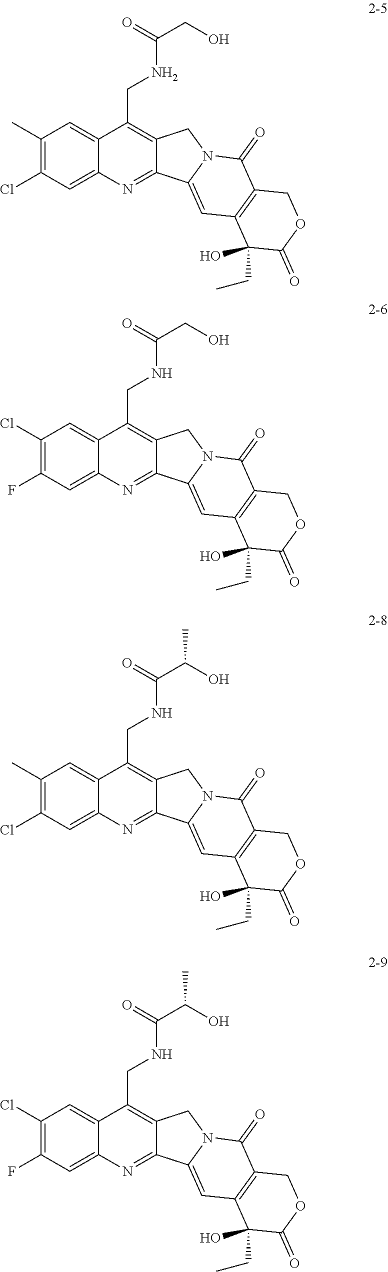

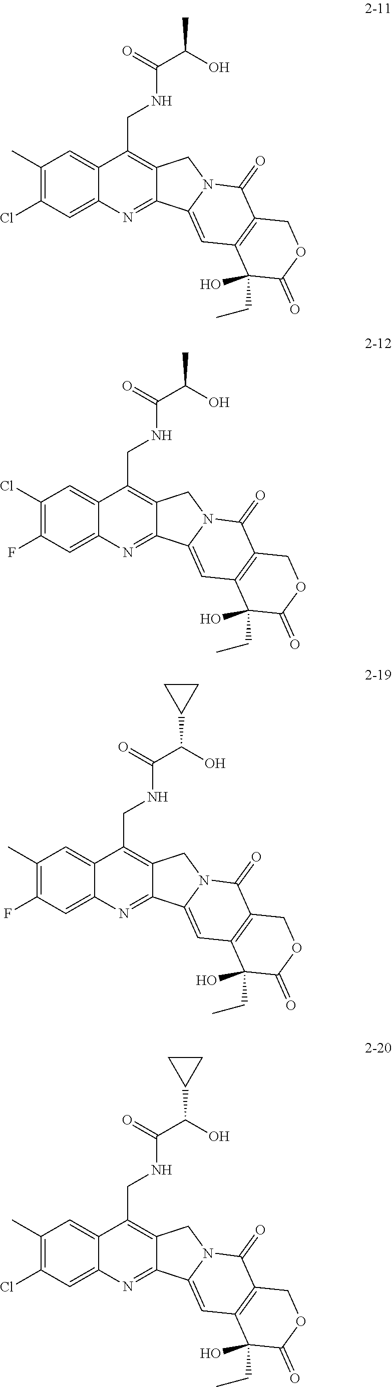

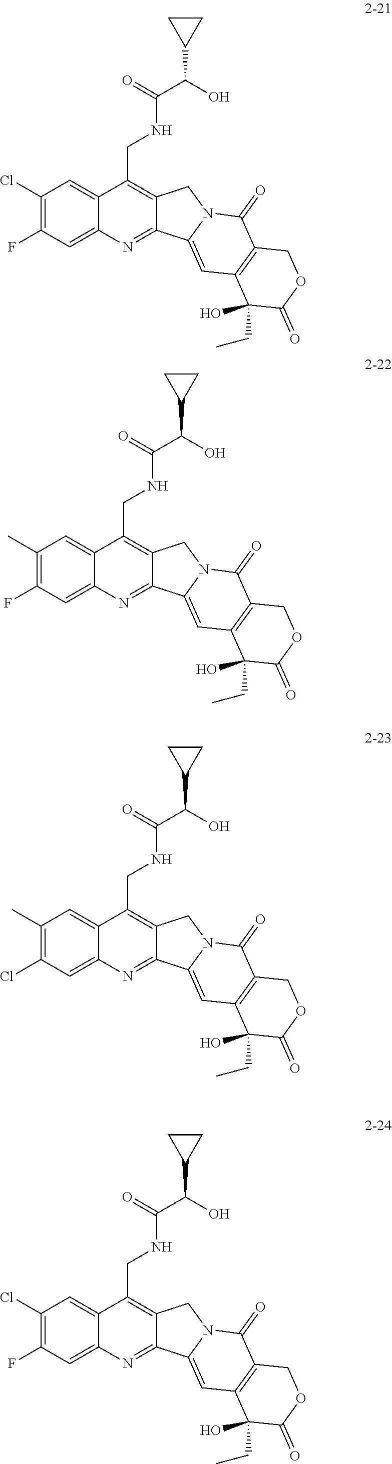

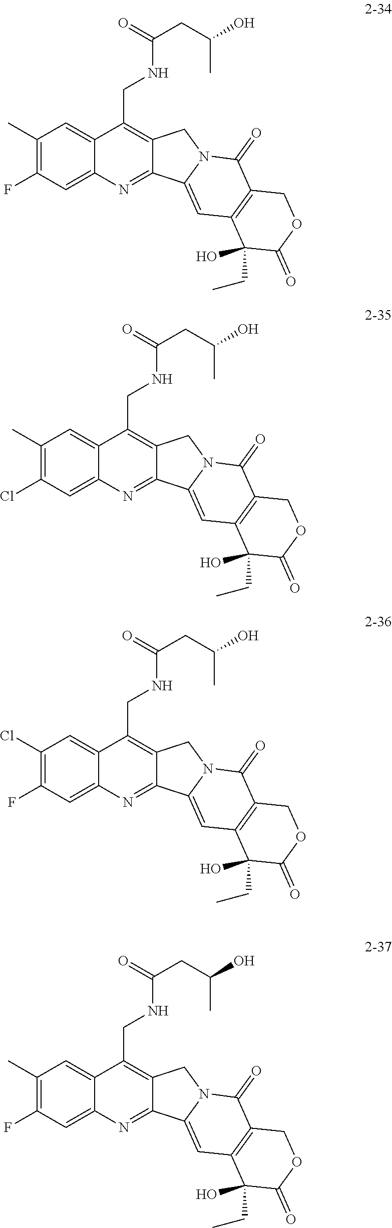 [SMILES: CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O]

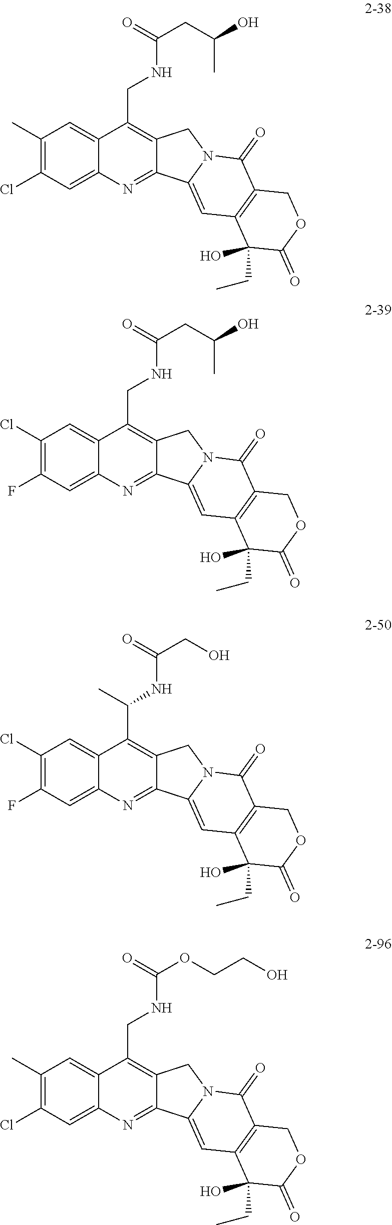

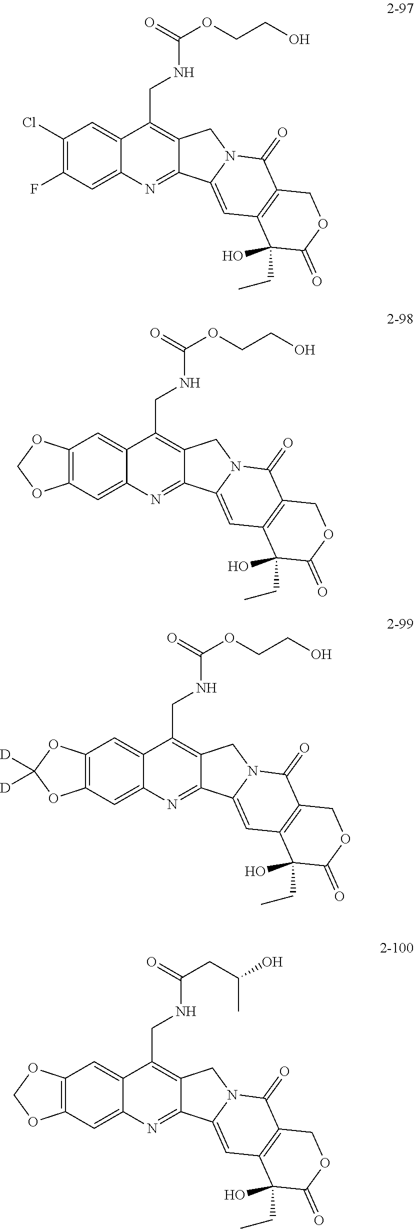

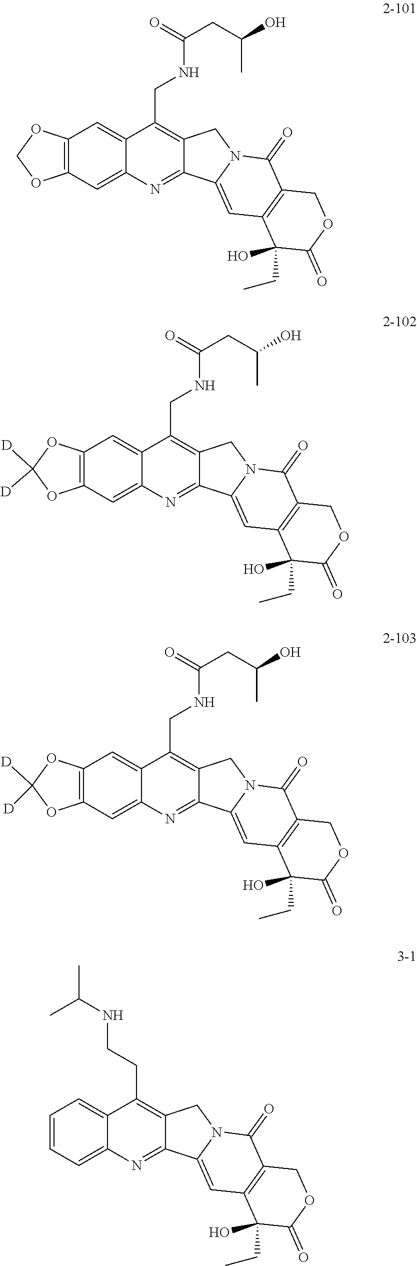 [SMILES: CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C]

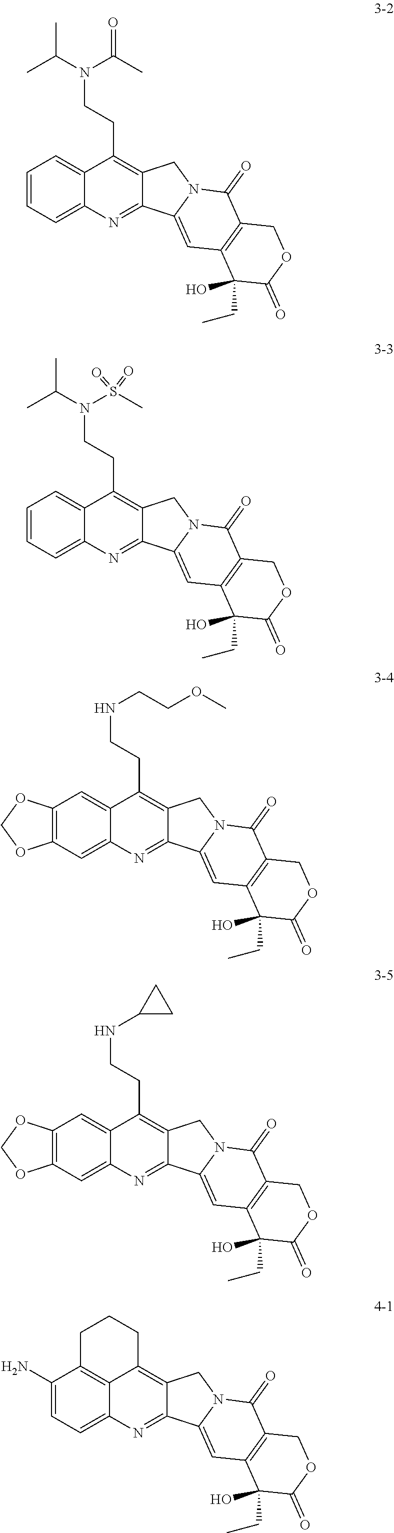

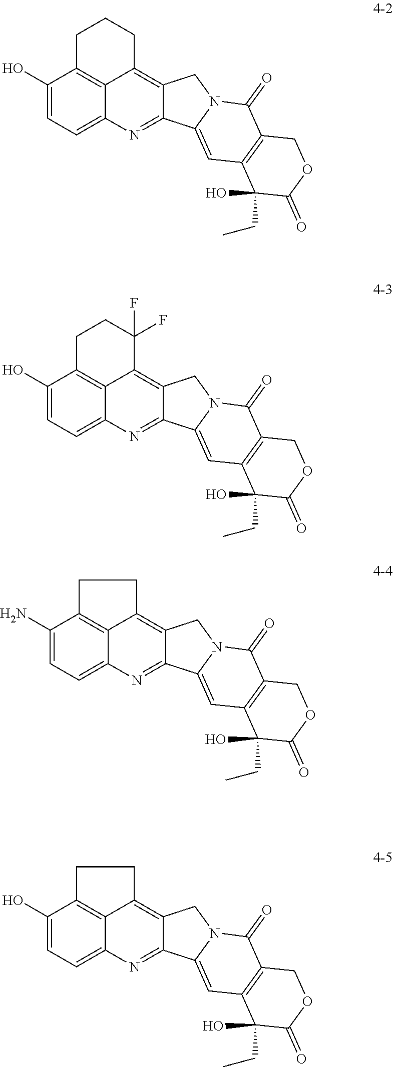 [SMILES: CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2C(F)(F)CC3 ; CC[C@@]1(O)C(=O)OCc2c1cc1n(c2=O)Cc2c-1nc1ccc(N)c3c1c2CC3 ; CC[C@@]1(O)C(=O)OCc2c1cc1n(c2=O)Cc2c-1nc1ccc(O)c3c1c2CC3]

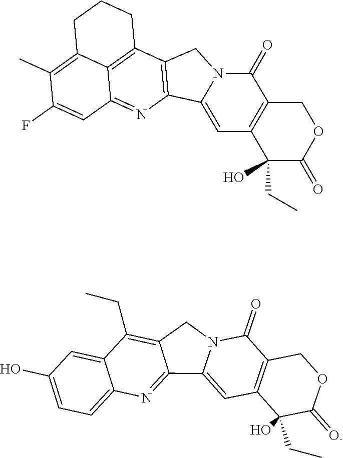 [SMILES: CC[C@@]1(O)C(=O)OCc2c1cc1n(c2=O)Cc2c-1nc1cc(F)c(C)c3c1c2CCC3 ; CCc1c2c(nc3ccc(O)cc13)-c1cc3c(c(=O)n1C2)COC(=O)[C@]3(O)CC]

In some embodiments, the cytotoxic drug is selected from the group consisting of the following compounds or isotopically labeled compounds thereof:

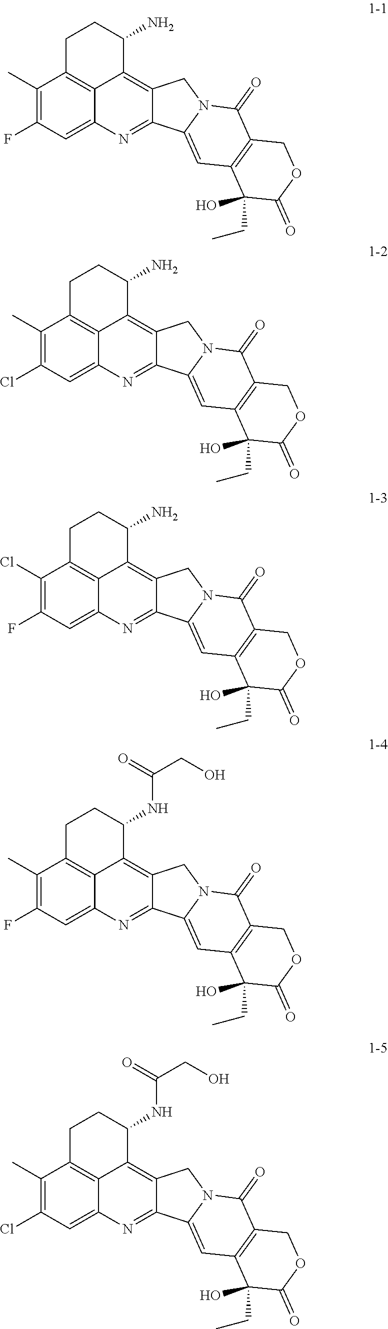

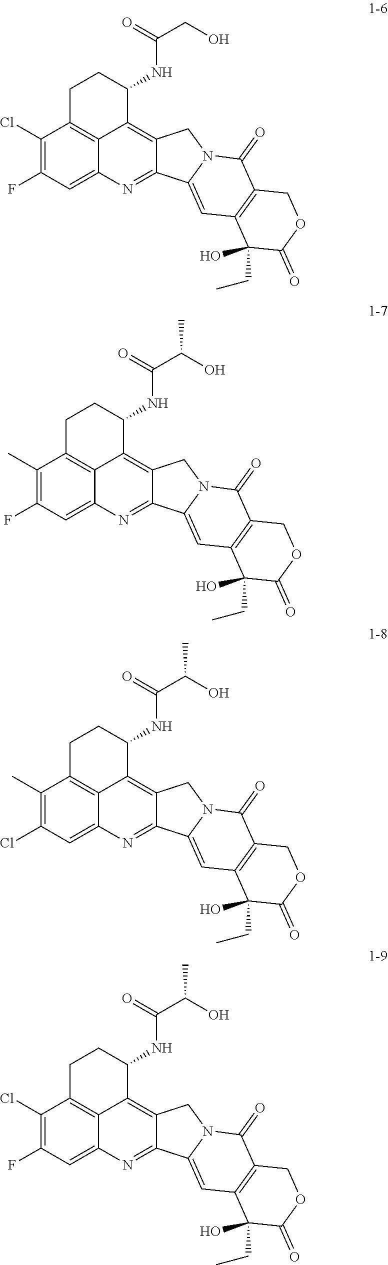

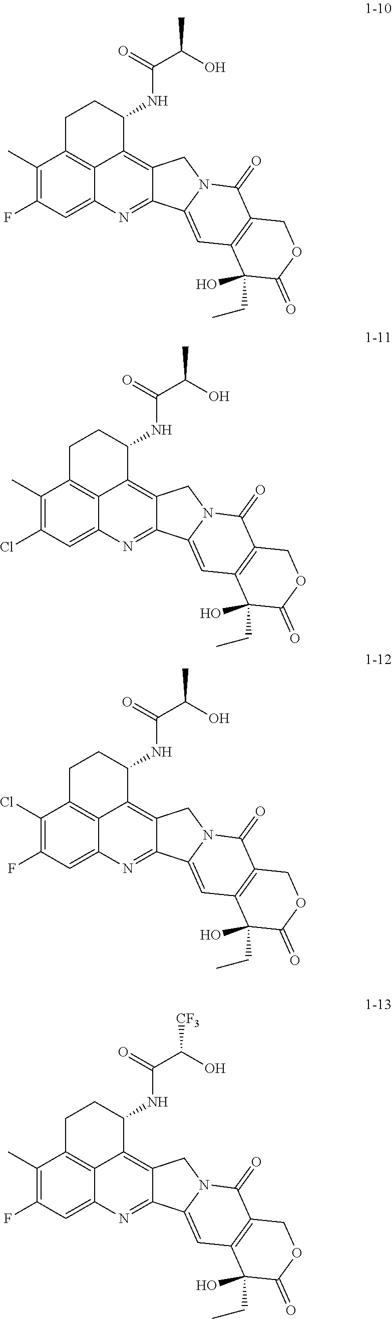

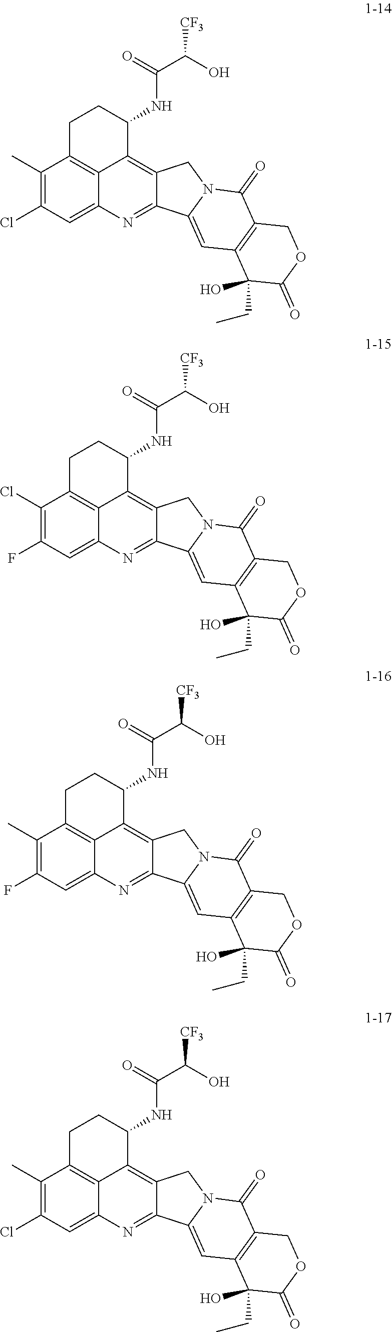 [SMILES: CC[C@@]1(O)C(=O)OCc2c1cc1n(c2=O)Cc2c-1nc1cc(Cl)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(C)c3c1c2[C@@H](NC(=O)[C@H](O)C(F)(F)F)CC3 ; CC[C@@]1(O)C(=O)OCc2c1cc1n(c2=O)Cc2c-1nc1cc(Cl)c(C)c3c1c2[C@@H](NC(=O)[C@H](O)C(F)(F)F)CC3]

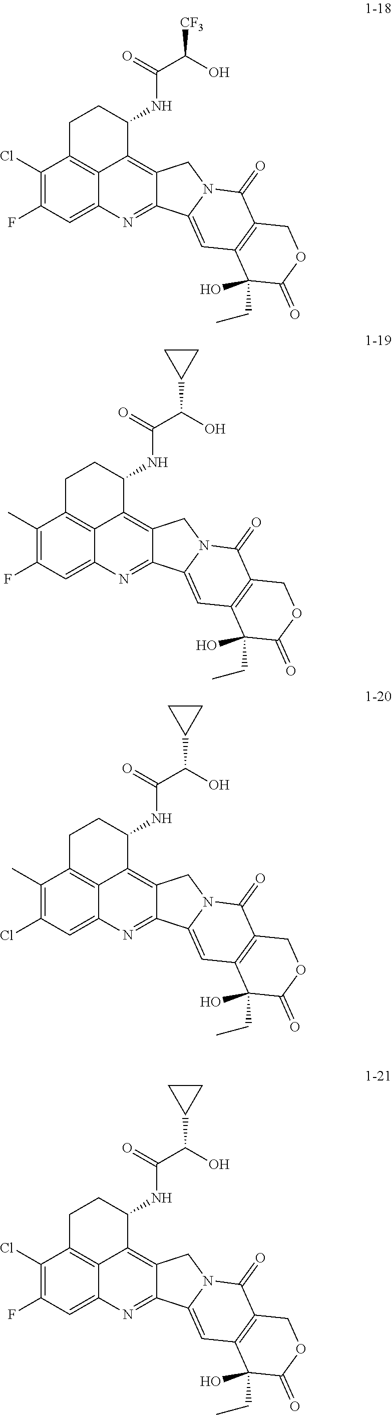

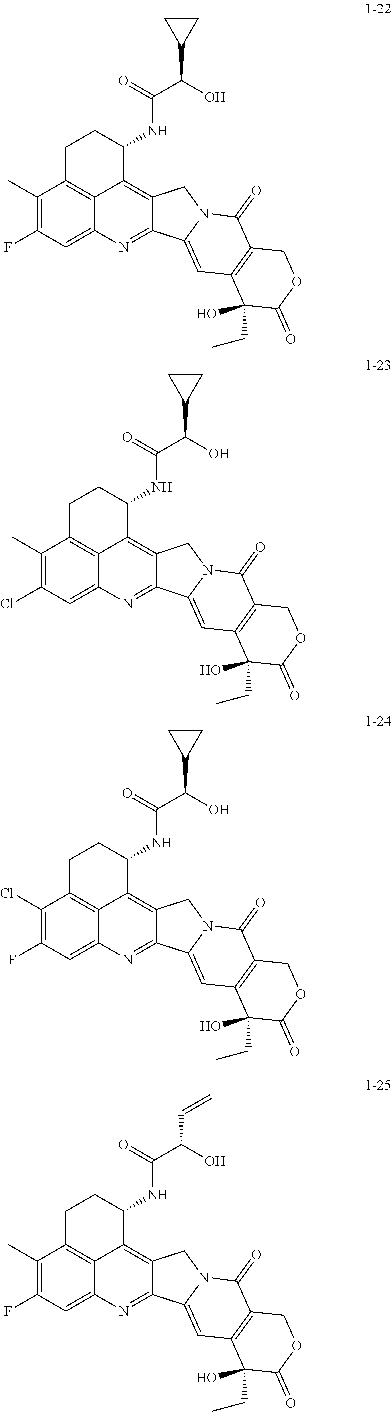

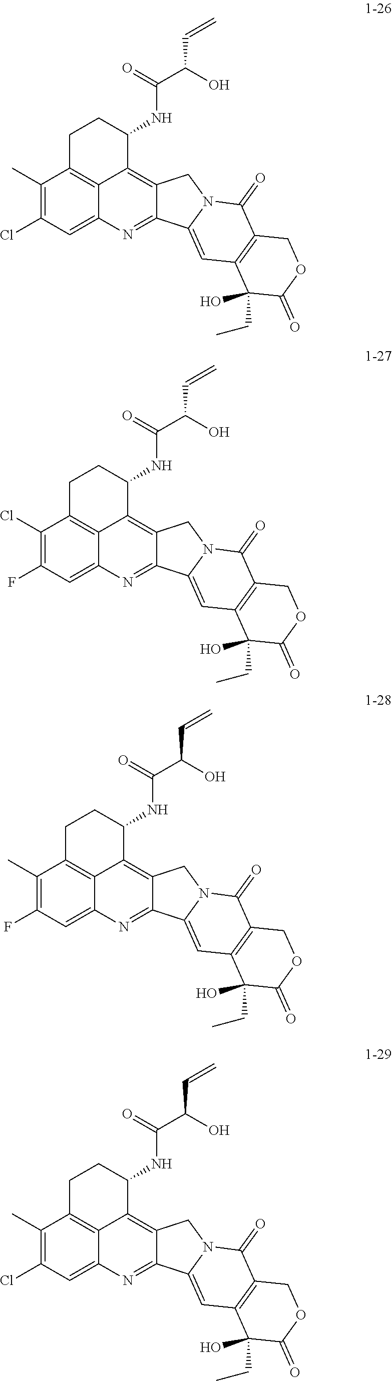

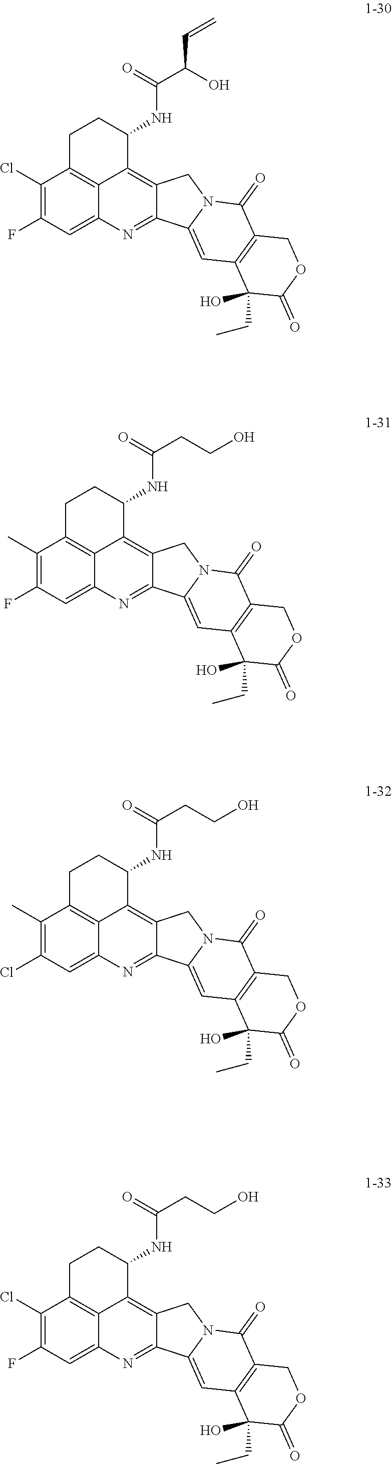 [SMILES: C=C[C@@H](O)C(=O)N[C@H]1CCc2c(Cl)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)CCO)CC3 ; CC[C@@]1(O)C(=O)OCc2c1cc1n(c2=O)Cc2c-1nc1cc(Cl)c(C)c3c1c2[C@@H](NC(=O)CCO)CC3 ; CC[C@@]1(O)C(=O)OCc2c1cc1n(c2=O)Cc2c-1nc1cc(F)c(Cl)c3c1c2[C@@H](NC(=O)CCO)CC3]

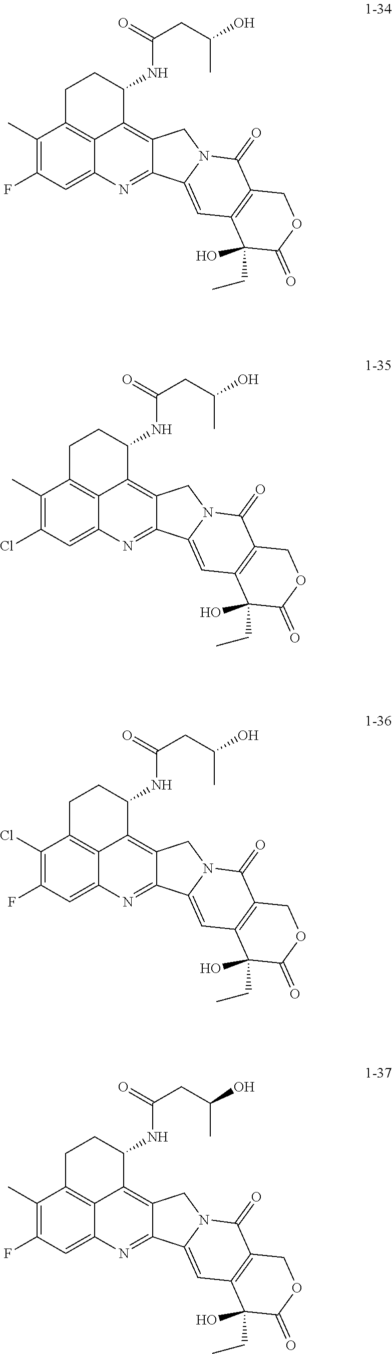 [SMILES: CC[C@@]1(O)C(=O)OCc2c1cc1n(c2=O)Cc2c-1nc1cc(F)c(C)c3c1c2[C@@H](NC(=O)C[C@@H](C)O)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(C)c3c1c2[C@@H](NC(=O)C[C@H](C)O)CC3]

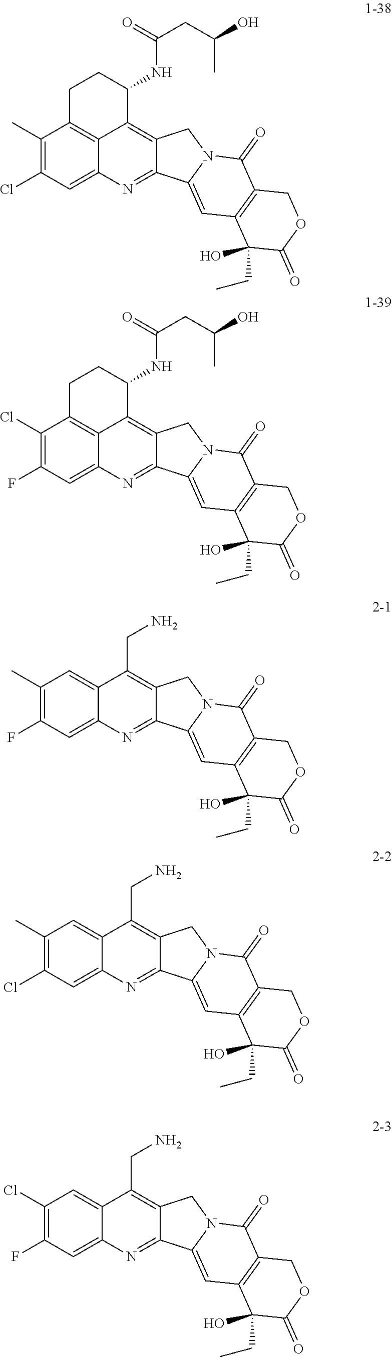

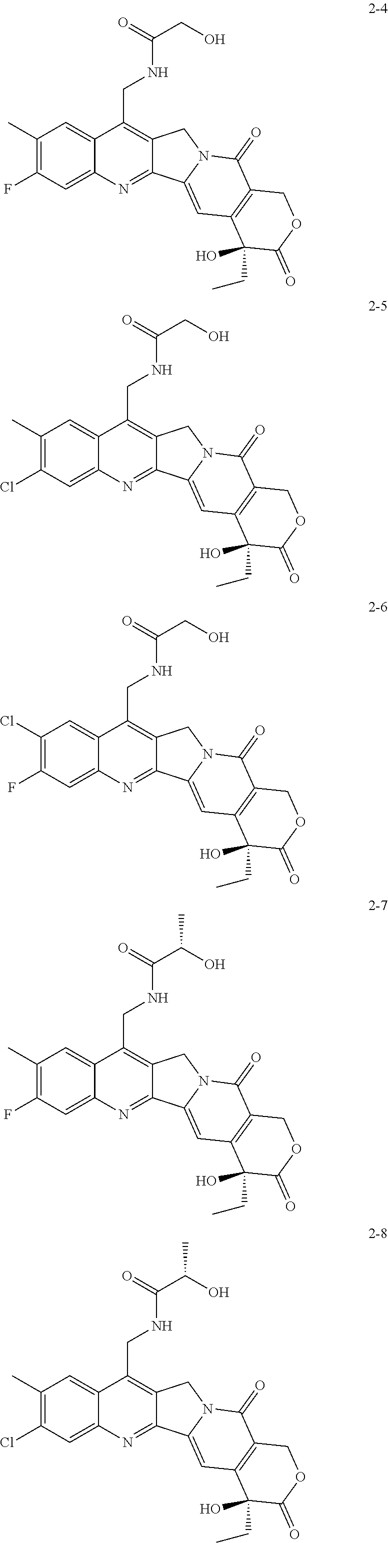

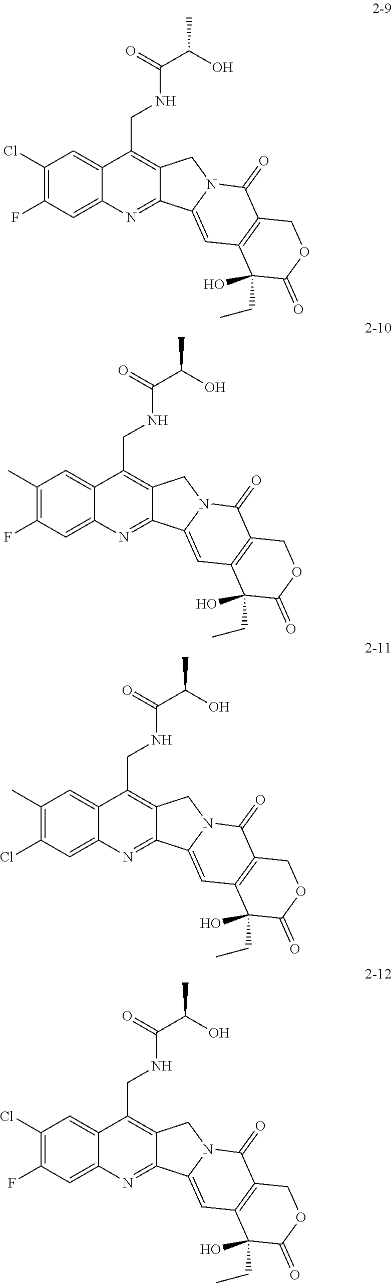

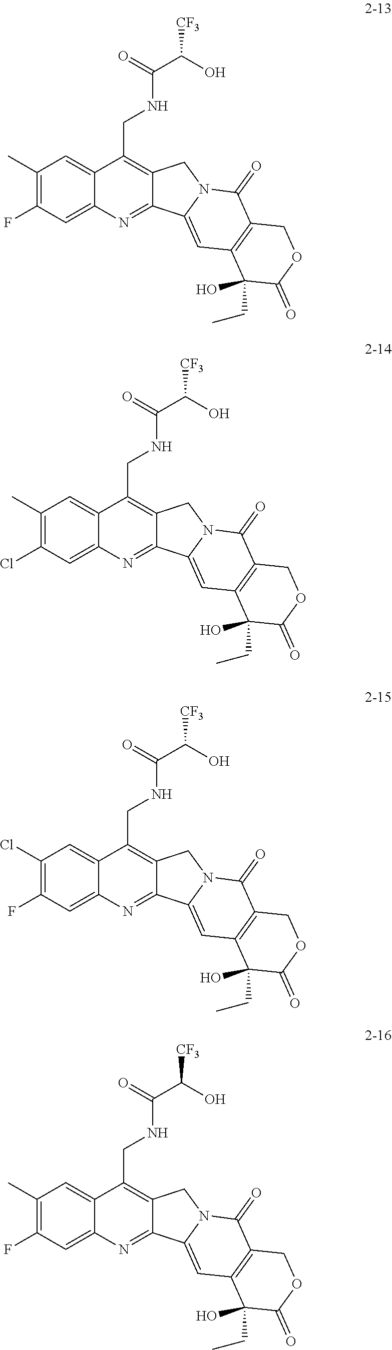 [SMILES: CC[C@@]1(O)C(=O)OCc2c1cc1n(c2=O)Cc2c-1nc1cc(F)c(C)cc1c2CNC(=O)[C@@H](O)C(F)(F)F ; CC[C@@]1(O)C(=O)OCc2c1cc1n(c2=O)Cc2c-1nc1cc(Cl)c(C)cc1c2CNC(=O)[C@@H](O)C(F)(F)F ; CC[C@@]1(O)C(=O)OCc2c1cc1n(c2=O)Cc2c-1nc1cc(F)c(Cl)cc1c2CNC(=O)[C@@H](O)C(F)(F)F ; CC[C@@]1(O)C(=O)OCc2c1cc1n(c2=O)Cc2c-1nc1cc(F)c(C)cc1c2CNC(=O)[C@H](O)C(F)(F)F]

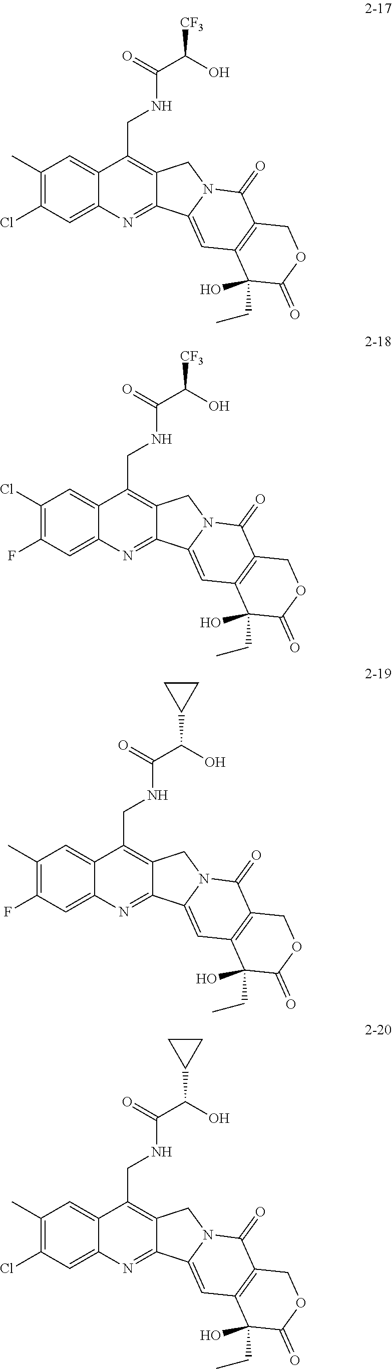

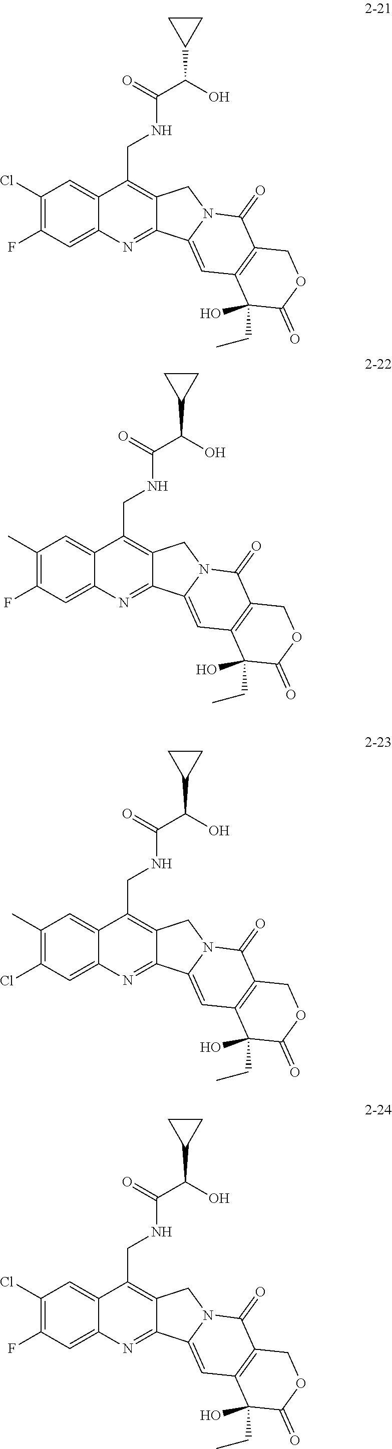

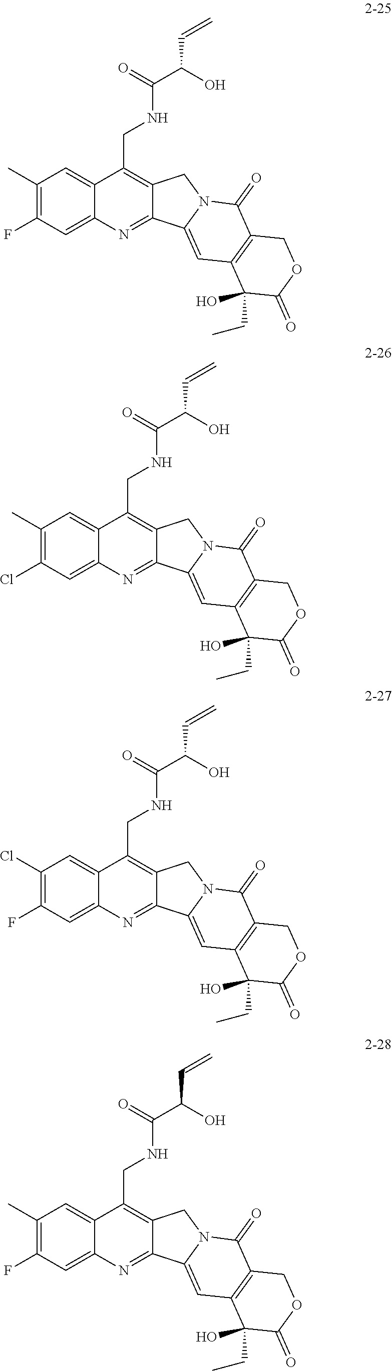

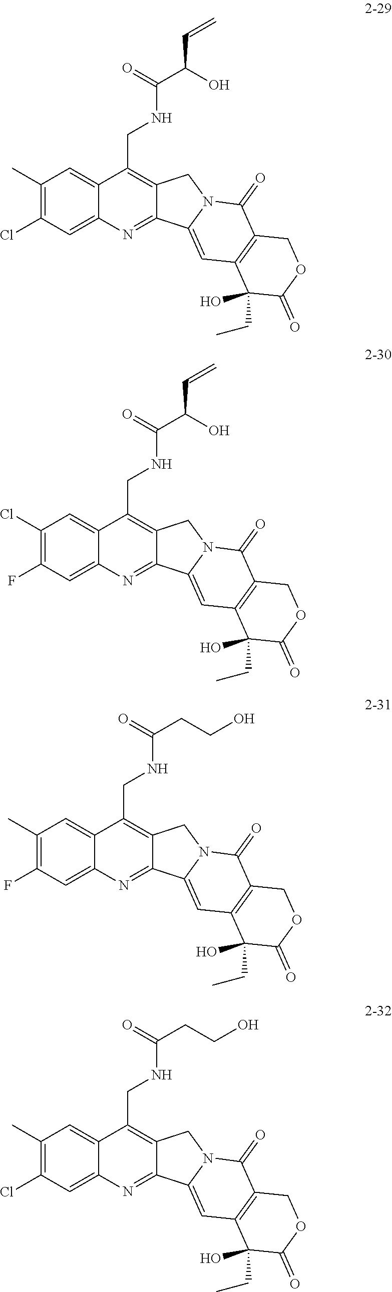

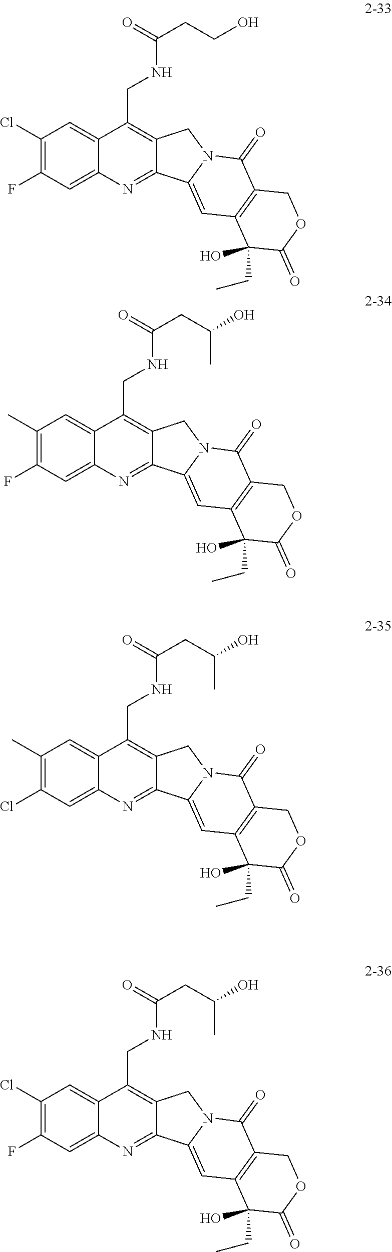

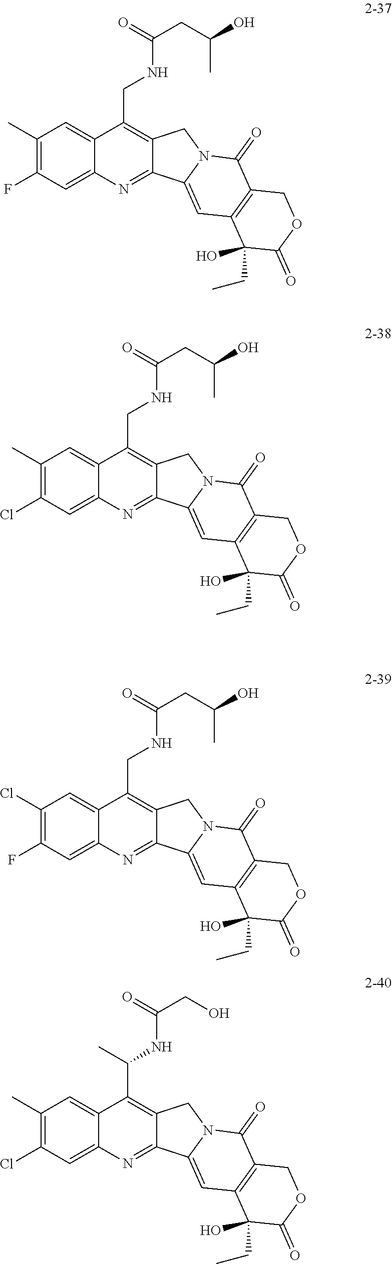 [SMILES: CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2[C@H](C)NC(=O)CO]

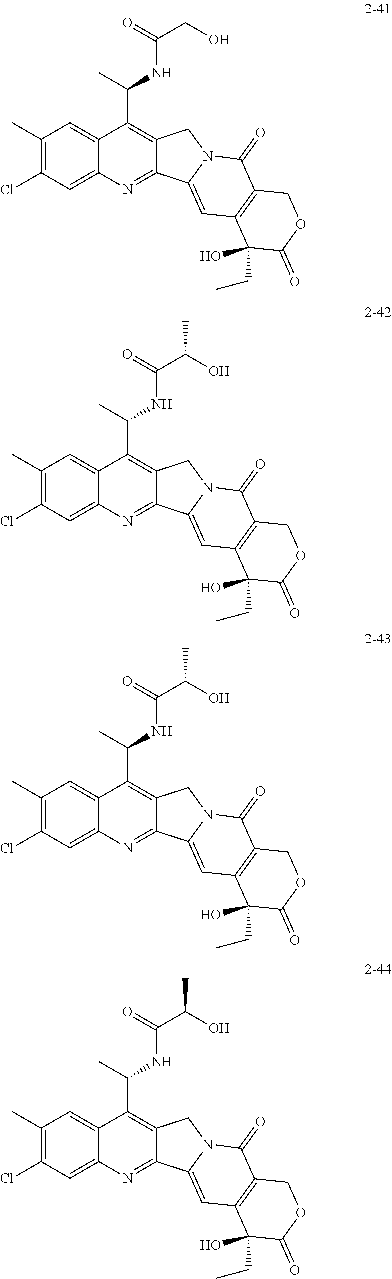

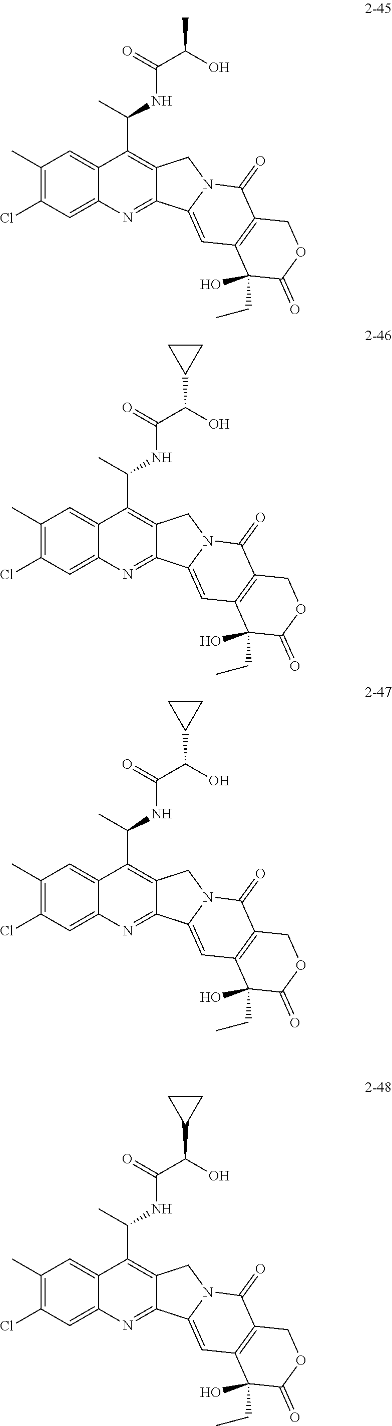

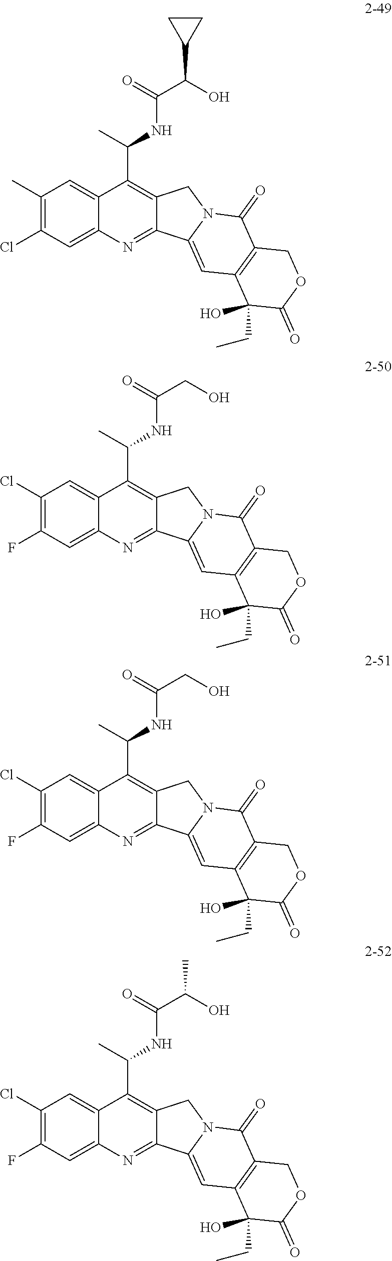

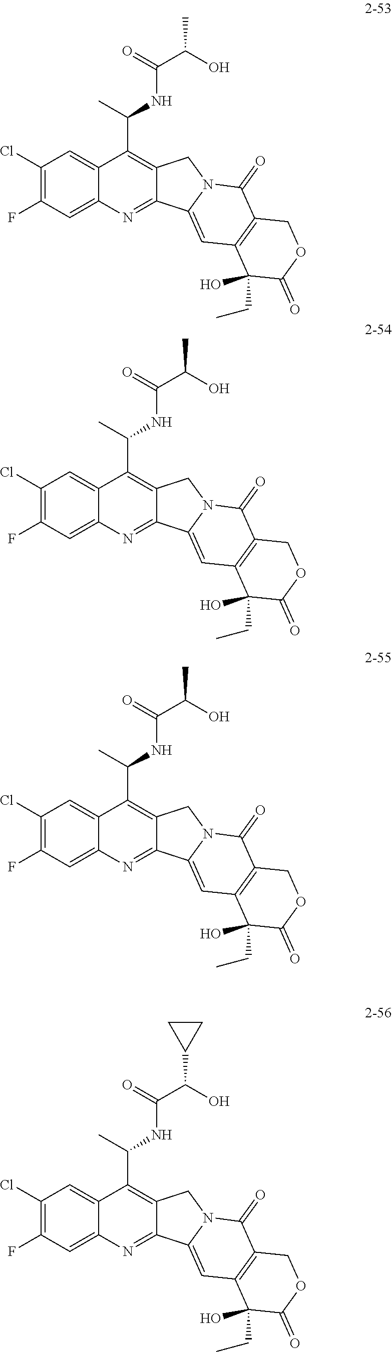

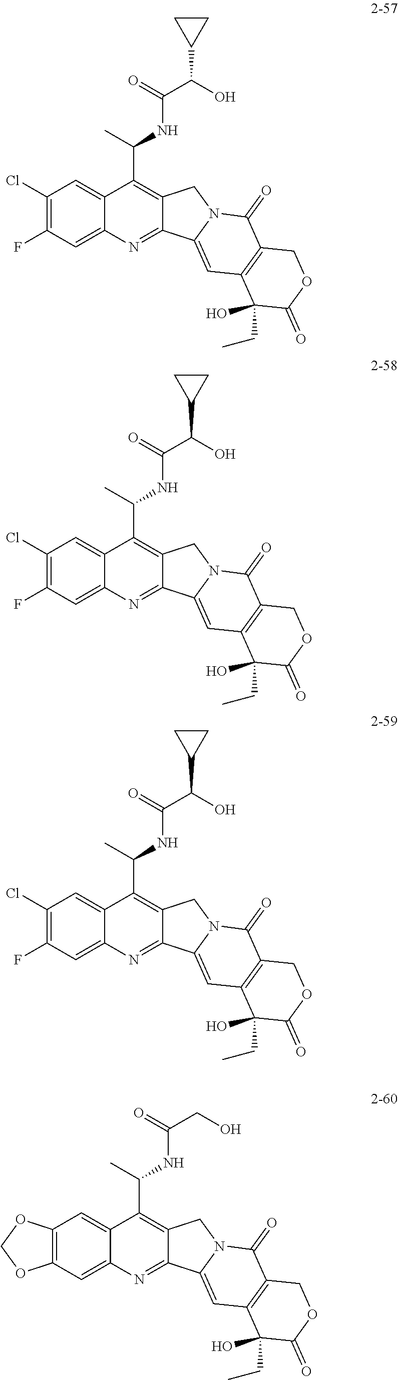

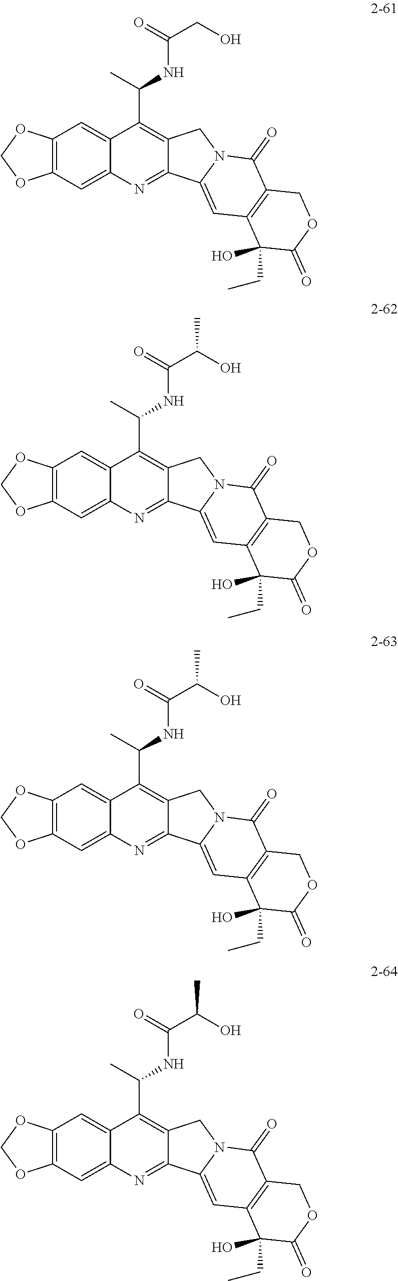

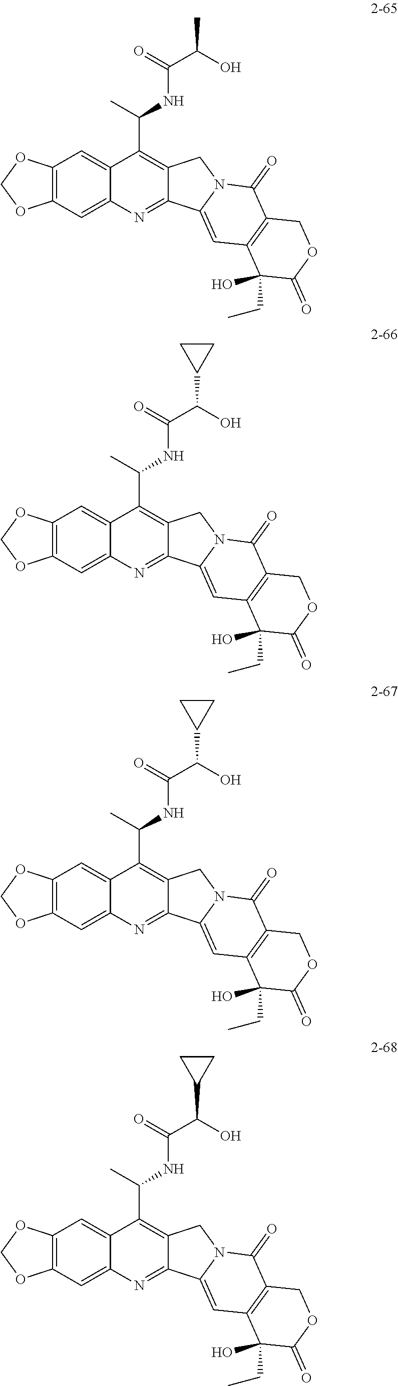

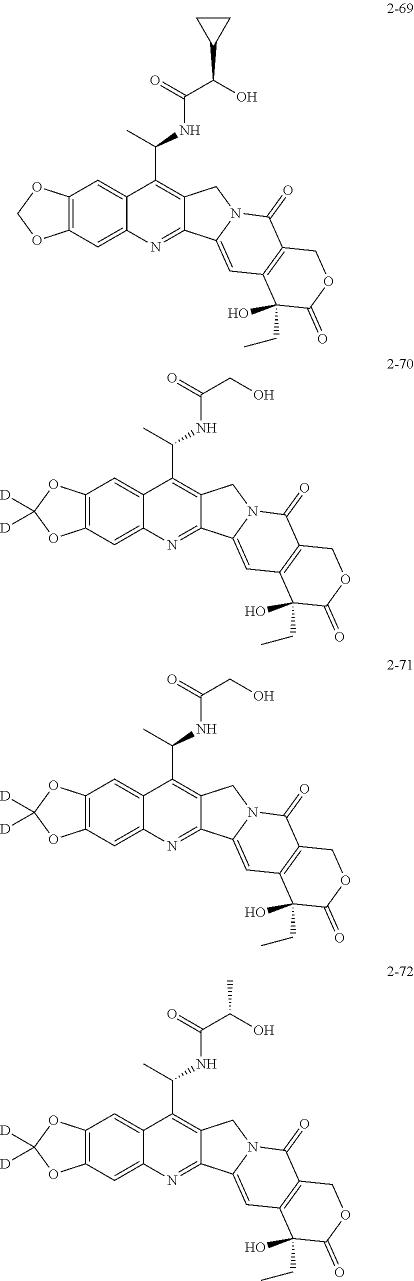

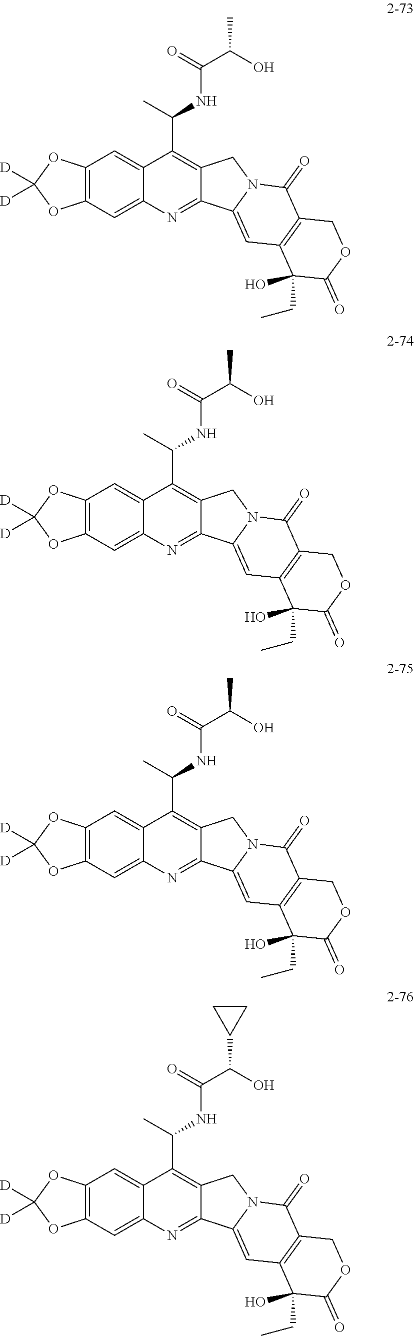

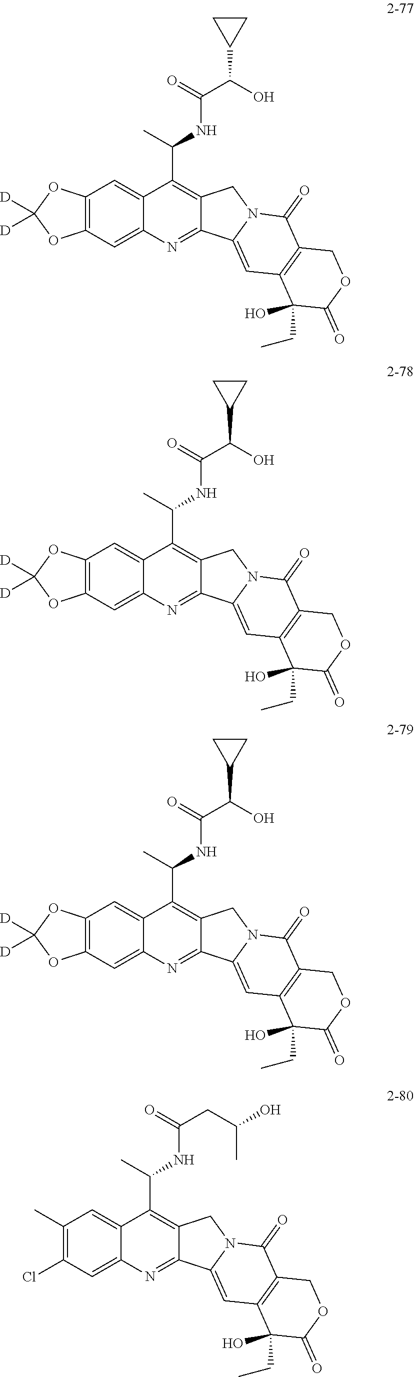

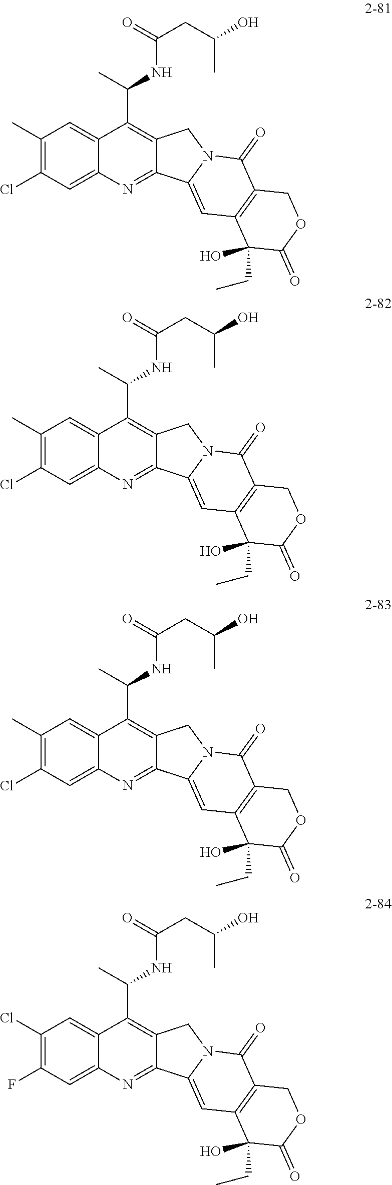

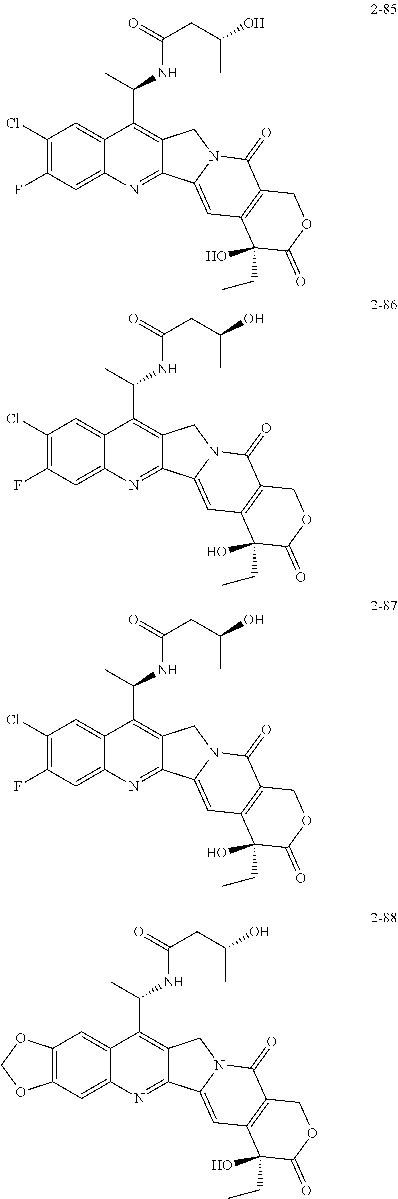

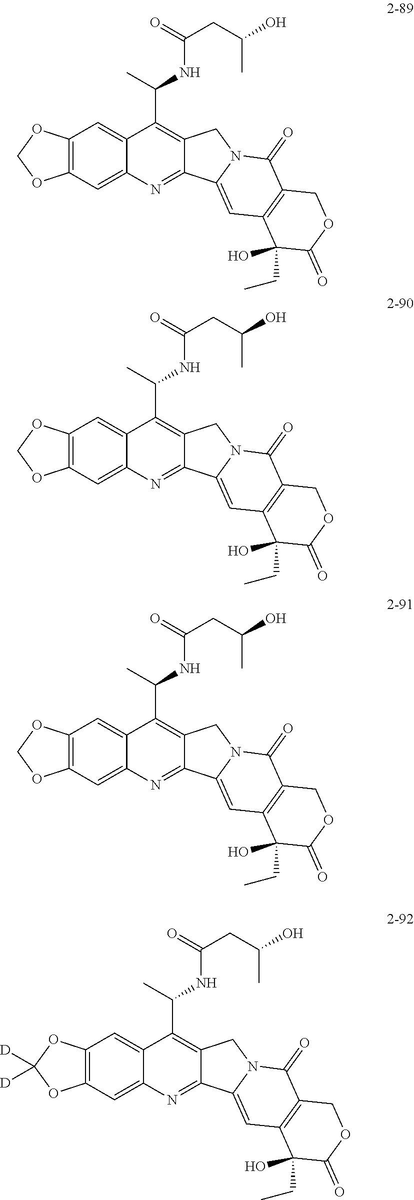

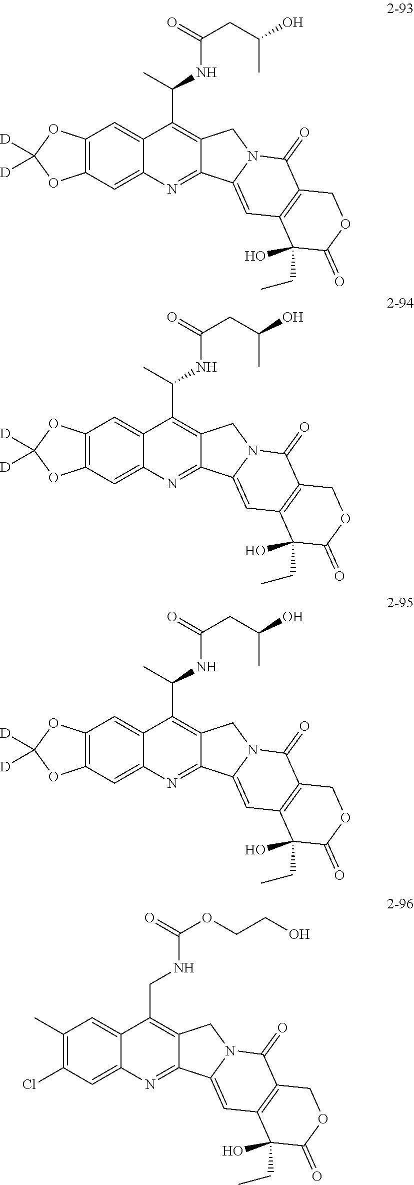

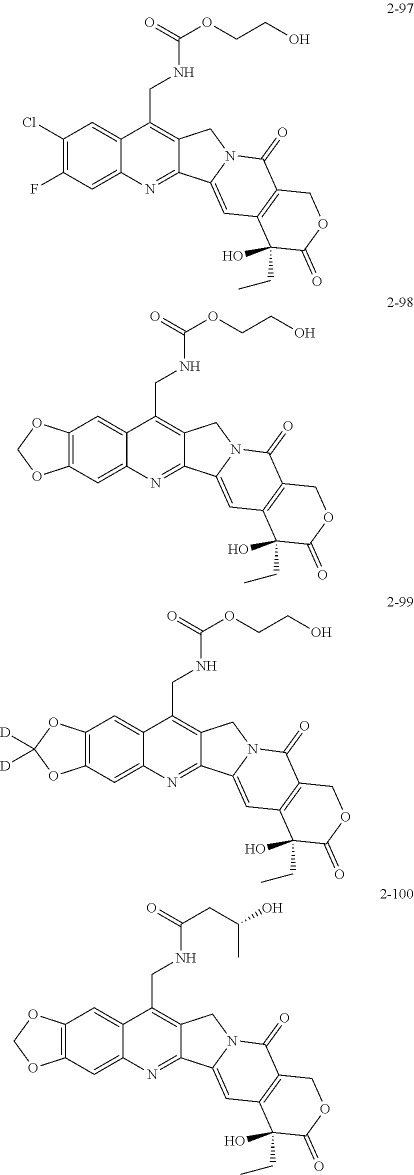

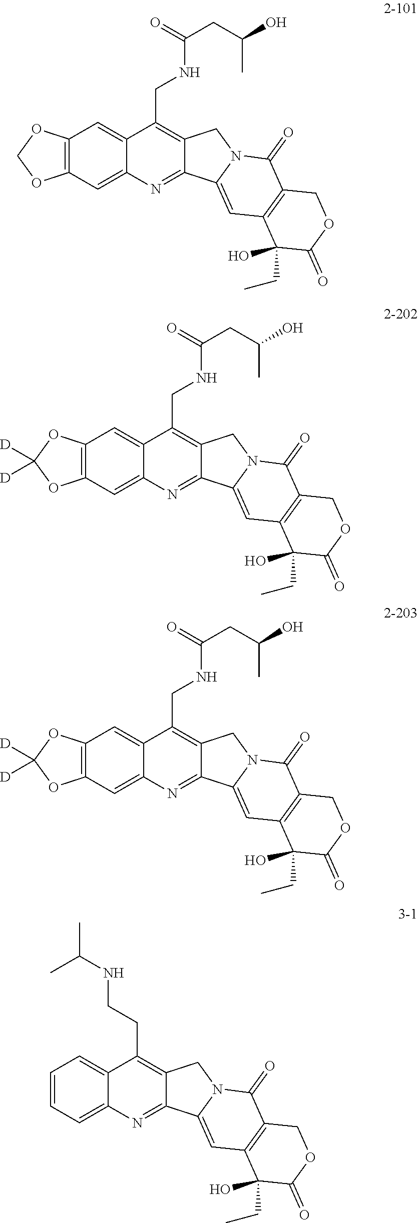

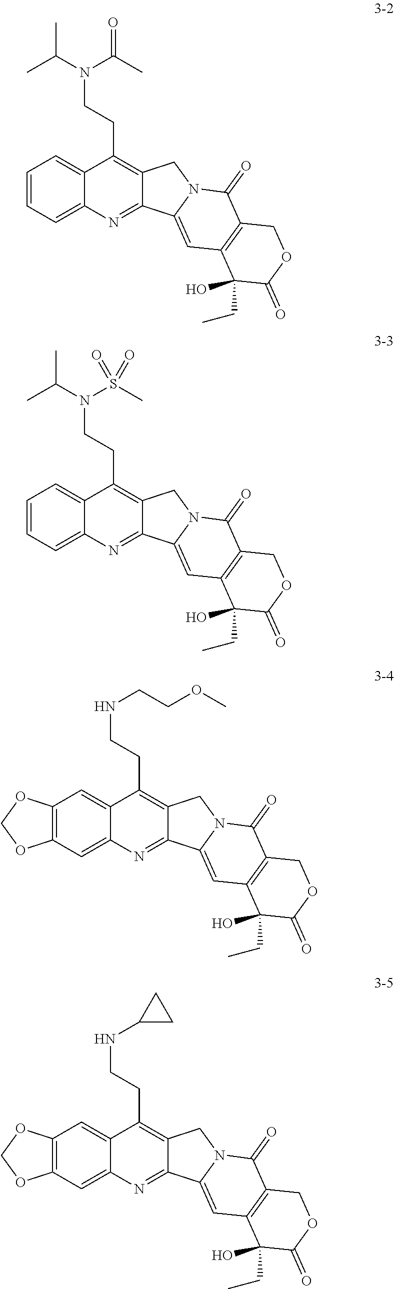

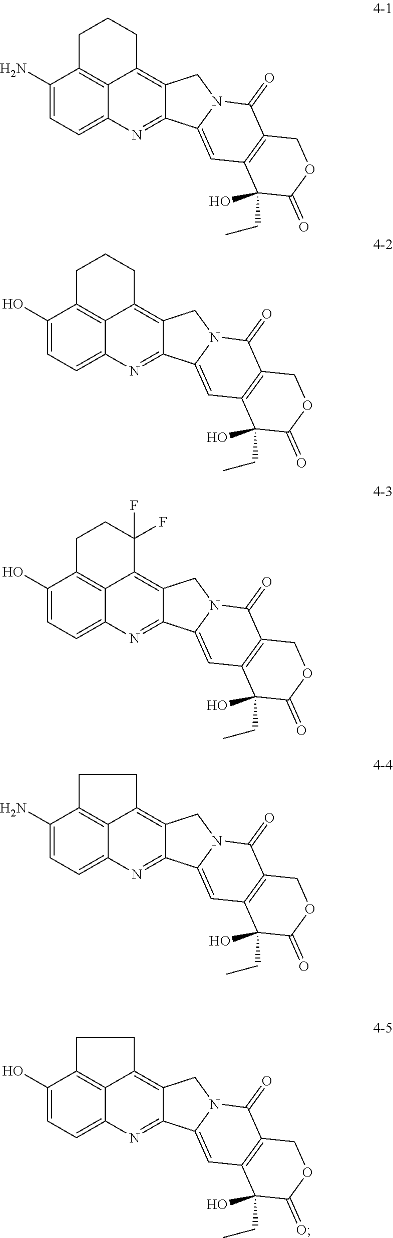 [SMILES: CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2C(F)(F)CC3 ; CC[C@@]1(O)C(=O)OCc2c1cc1n(c2=O)Cc2c-1nc1ccc(N)c3c1c2CC3 ; CC[C@@]1(O)C(=O)OCc2c1cc1n(c2=O)Cc2c-1nc1ccc(O)c3c1c2CC3]

preferably, the cytotoxic drug is selected from the group consisting of the following compounds or isotopically labeled compounds thereof:

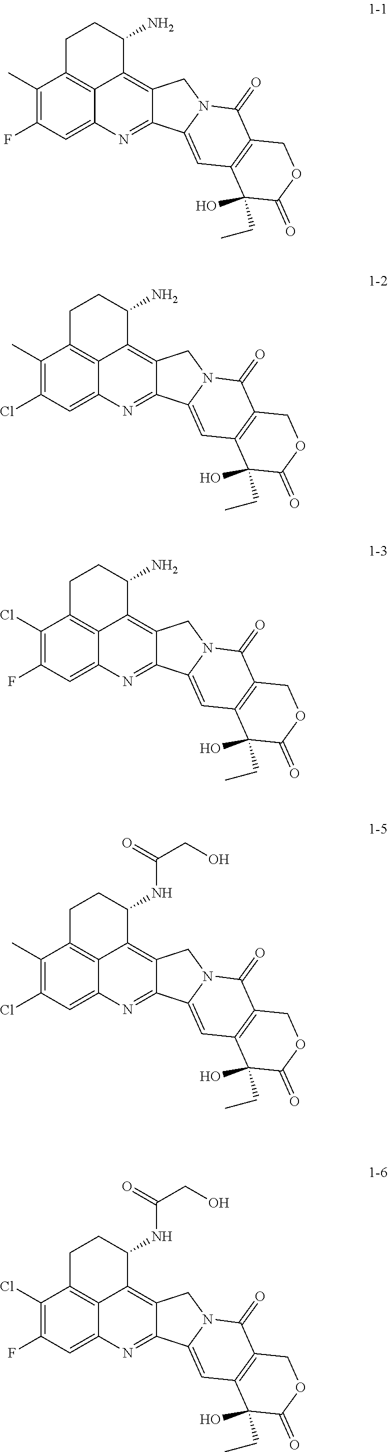 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3]

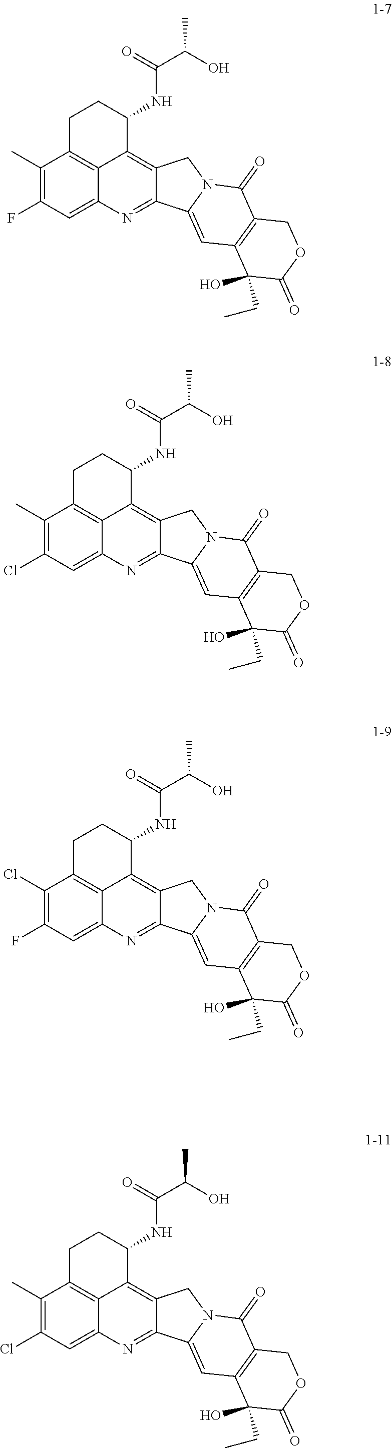 [SMILES: CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3]

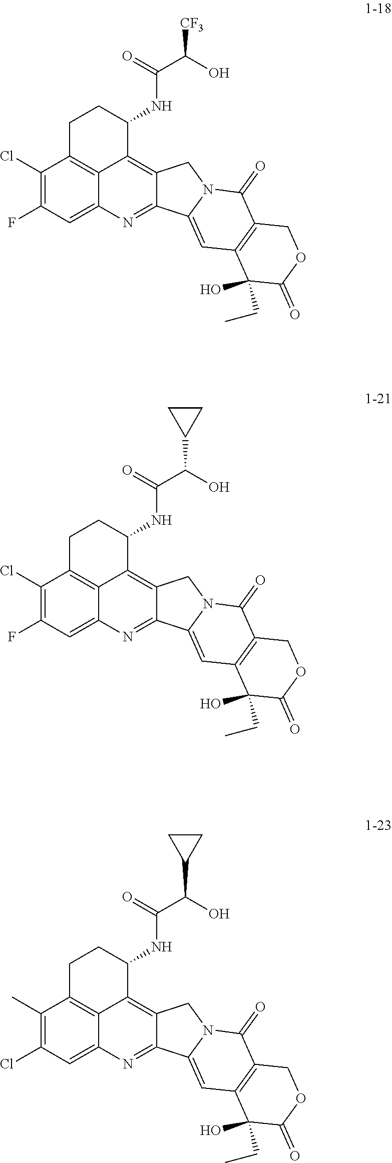 [SMILES: CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3]

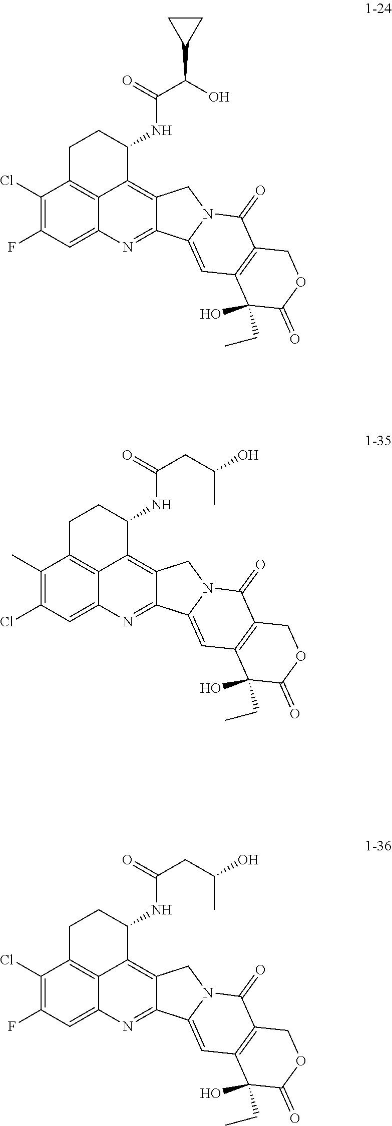 [SMILES: CC[C@@]1(O)C(=O)OCc2c1cc1n(c2=O)Cc2c-1nc1cc(F)c(Cl)c3c1c2[C@@H](NC(=O)[C@H](O)C1CC1)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3]

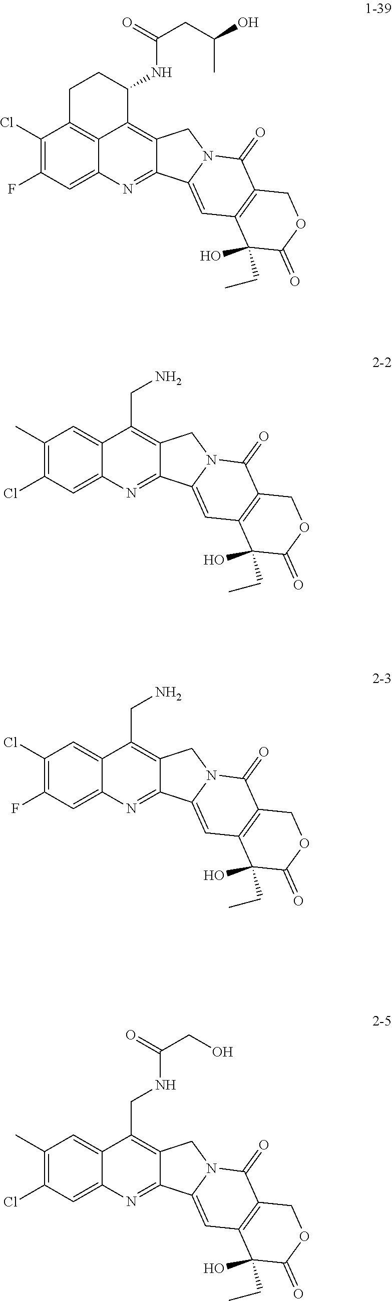 [SMILES: CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(Cl)c(C)cc1c2CNC(=O)CO]

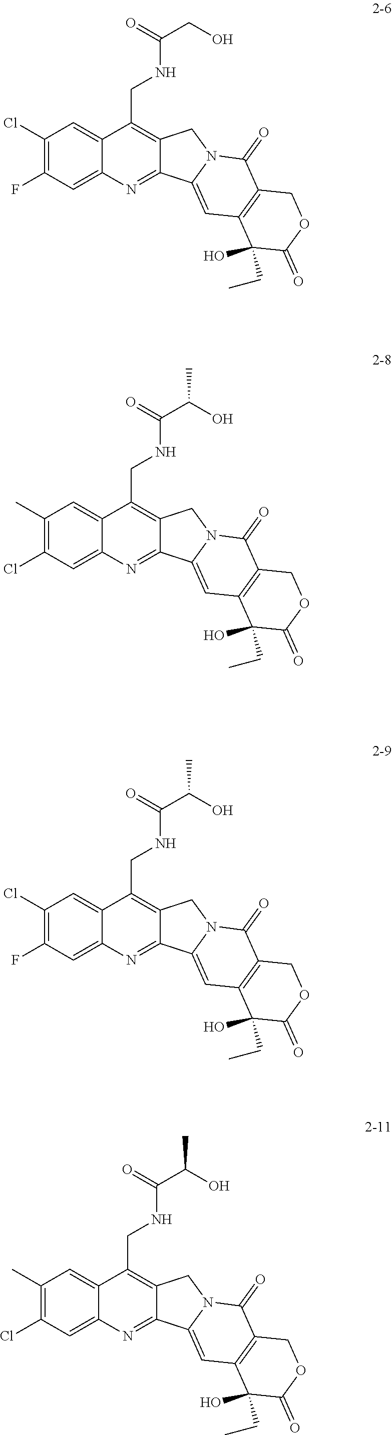 [SMILES: CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@@H](C)O]

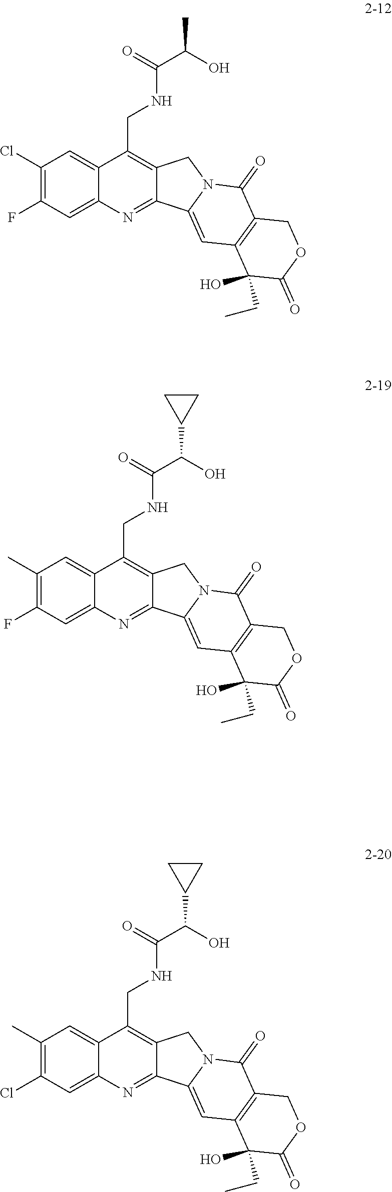 [SMILES: CC[C@@]1(O)C(=O)OCc2c1cc1n(c2=O)Cc2c-1nc1cc(F)c(Cl)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](O)C1CC1 ; CC[C@@]1(O)C(=O)OCc2c1cc1n(c2=O)Cc2c-1nc1cc(Cl)c(C)cc1c2CNC(=O)[C@@H](O)C1CC1]

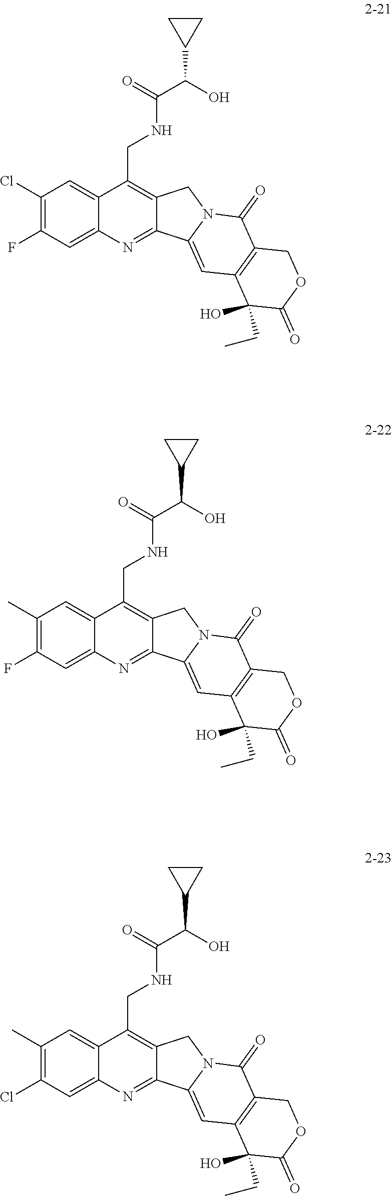 [SMILES: CC[C@@]1(O)C(=O)OCc2c1cc1n(c2=O)Cc2c-1nc1cc(F)c(Cl)cc1c2CNC(=O)[C@@H](O)C1CC1 ; CC[C@@]1(O)C(=O)OCc2c1cc1n(c2=O)Cc2c-1nc1cc(F)c(C)cc1c2CNC(=O)[C@H](O)C1CC1 ; CC[C@@]1(O)C(=O)OCc2c1cc1n(c2=O)Cc2c-1nc1cc(Cl)c(C)cc1c2CNC(=O)[C@H](O)C1CC1]

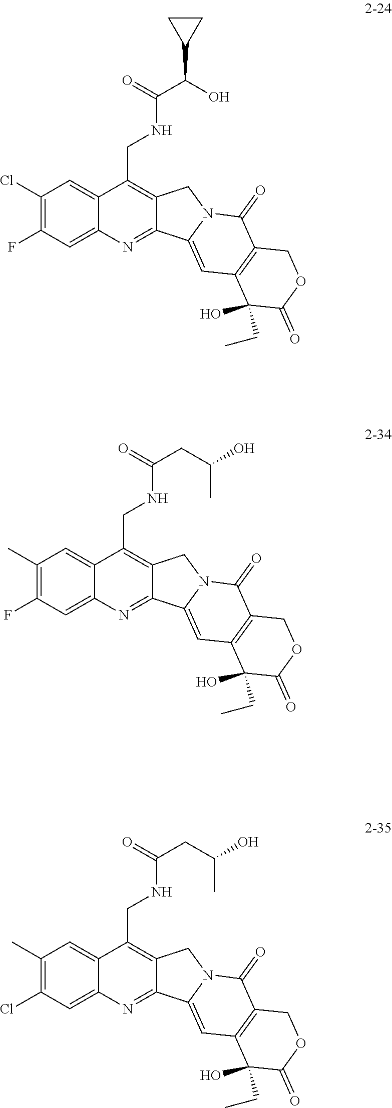 [SMILES: CC[C@@]1(O)C(=O)OCc2c1cc1n(c2=O)Cc2c-1nc1cc(F)c(Cl)cc1c2CNC(=O)[C@H](O)C1CC1 ; CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O]

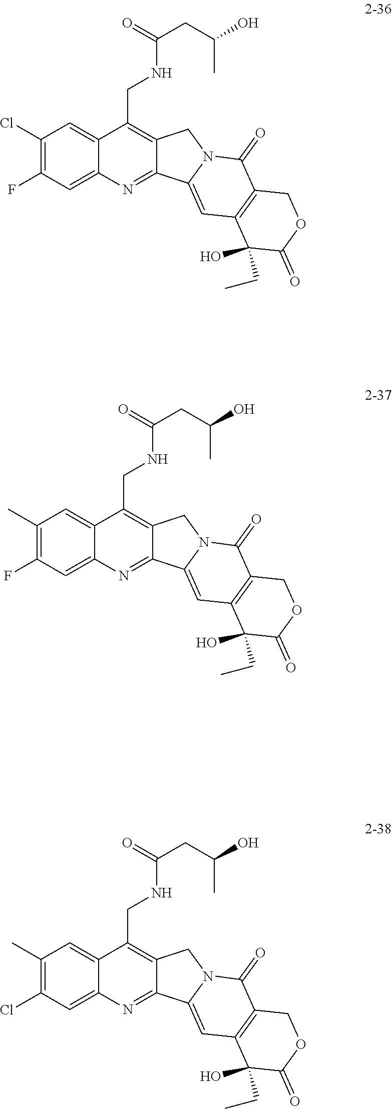 [SMILES: CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O]

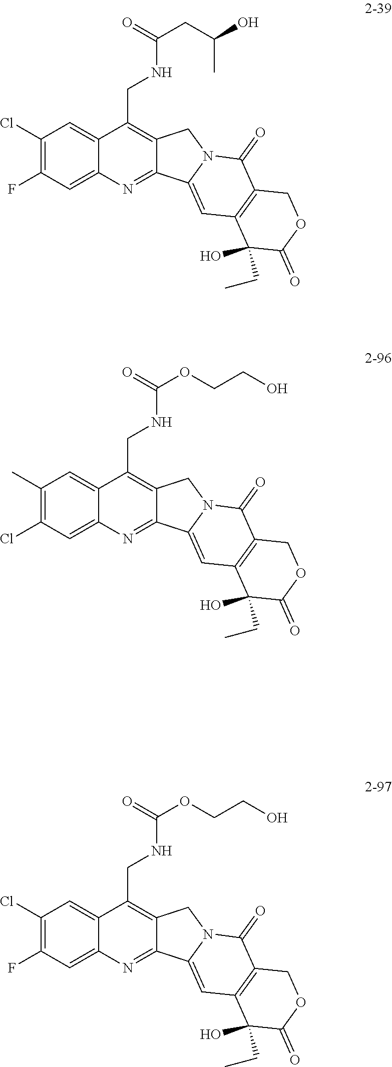 [SMILES: CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2CNC(=O)OCCO ; CC[C@@]1(O)C(=O)OCc2c1cc1n(c2=O)Cc2c-1nc1cc(F)c(Cl)cc1c2CNC(=O)OCCO]

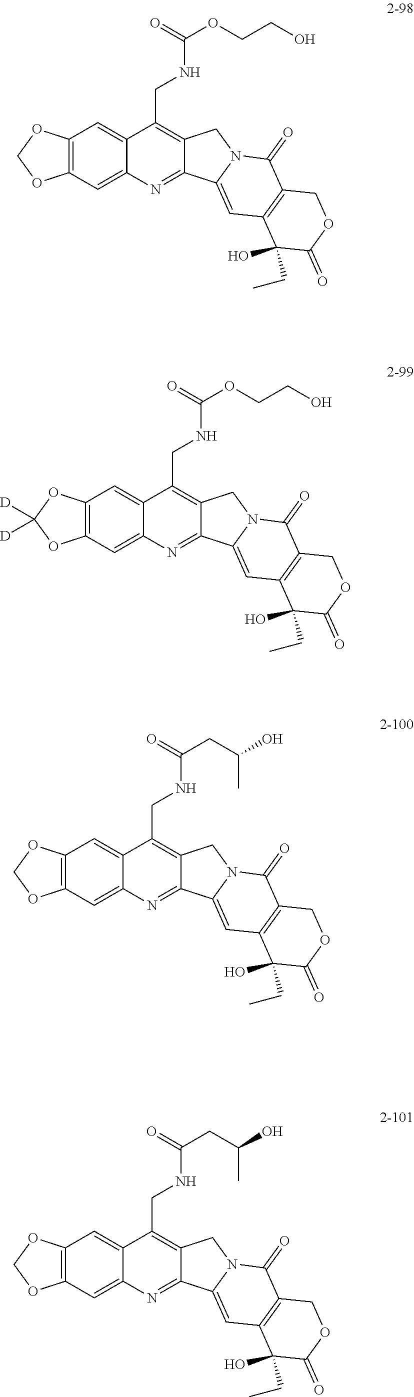 [SMILES: CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC ; CC[C@@]1(O)C(=O)OCc2c1cc1n(c2=O)Cc2c-1nc1cc3c(cc1c2CNC(=O)C[C@@H](C)O)OCO3 ; CC[C@@]1(O)C(=O)OCc2c1cc1n(c2=O)Cc2c-1nc1cc3c(cc1c2CNC(=O)C[C@H](C)O)OCO3]

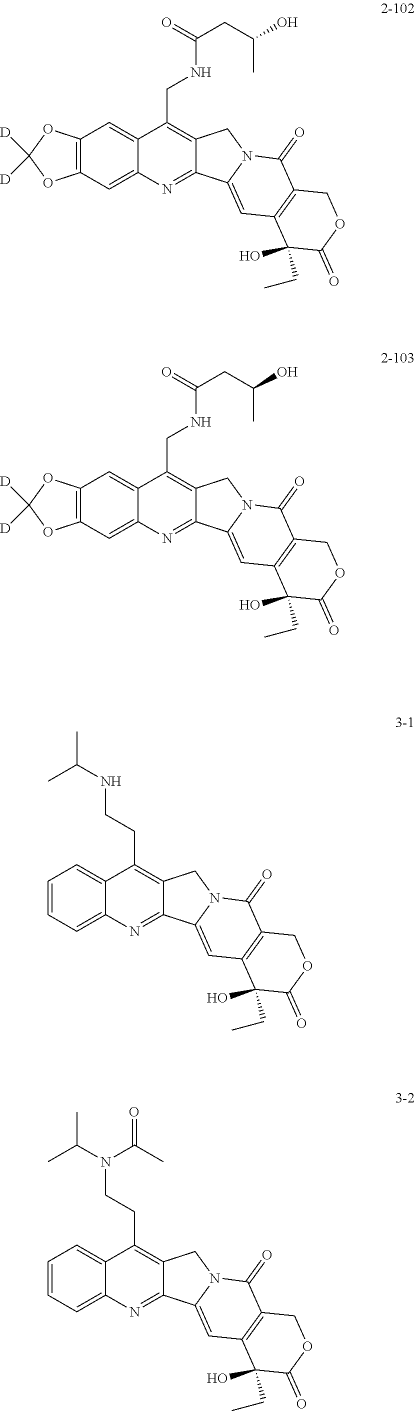 [SMILES: [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C]

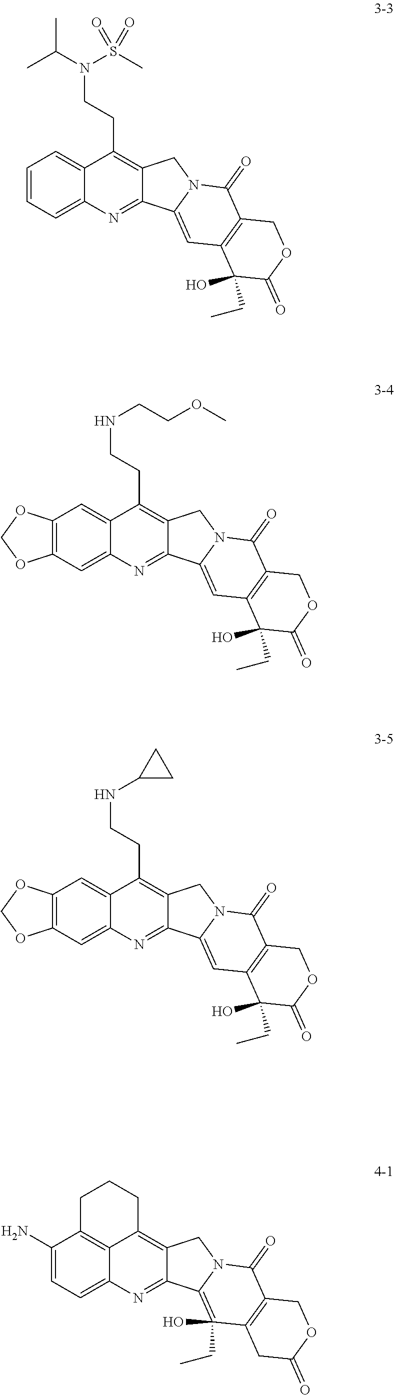

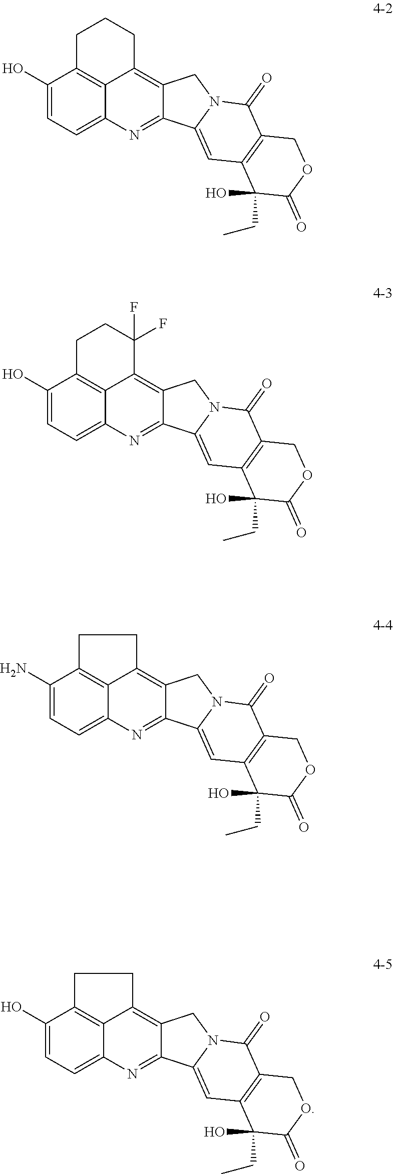 [SMILES: CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2C(F)(F)CC3 ; CC[C@@]1(O)C(=O)OCc2c1cc1n(c2=O)Cc2c-1nc1ccc(N)c3c1c2CC3 ; CC[C@@]1(O)C(=O)OCc2c1cc1n(c2=O)Cc2c-1nc1ccc(O)c3c1c2CC3]

In some embodiments, the cytotoxic drug is selected from the group consisting of the following compounds or isotopically labeled compounds thereof:

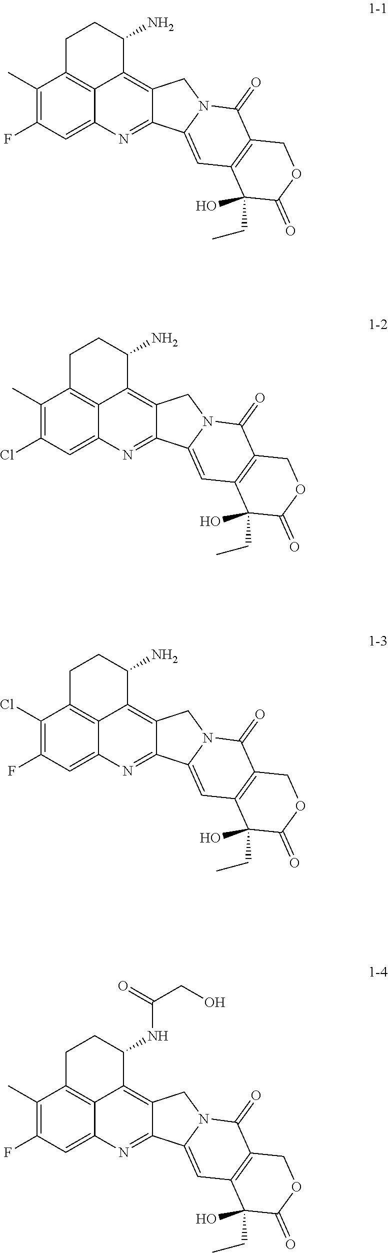 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3]

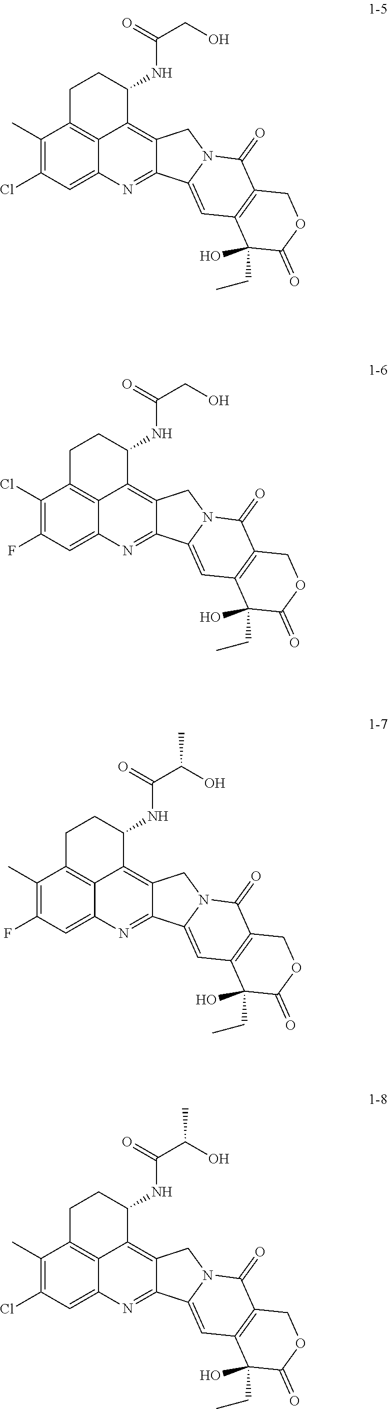 [SMILES: CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3]

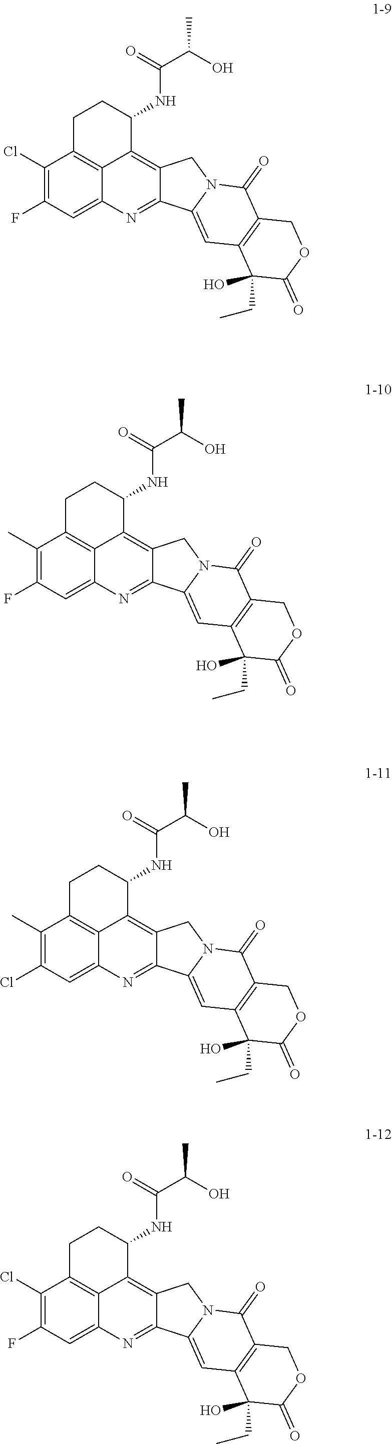 [SMILES: CC[C@@]1(O)C(=O)OCc2c1cc1n(c2=O)Cc2c-1nc1cc(F)c(Cl)c3c1c2[C@@H](NC(=O)[C@H](C)O)CC3 ; CC[C@@]1(O)C(=O)OCc2c1cc1n(c2=O)Cc2c-1nc1cc(F)c(C)c3c1c2[C@@H](NC(=O)[C@@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@@H](C)O)CC3]

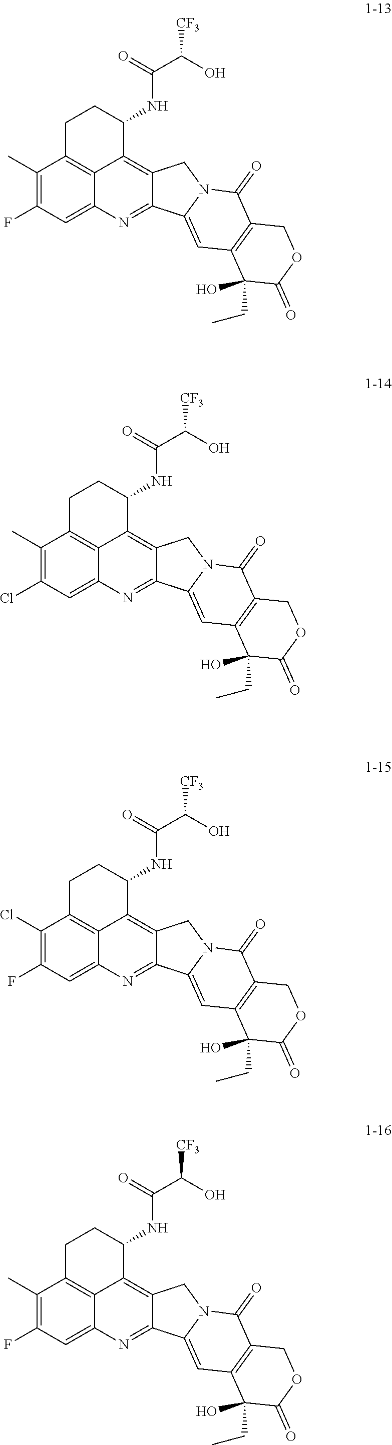 [SMILES: CC[C@@]1(O)C(=O)OCc2c1cc1n(c2=O)Cc2c-1nc1cc(F)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(Cl)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(C)c3c1c2[C@@H](NC(=O)[C@H](O)C(F)(F)F)CC3]

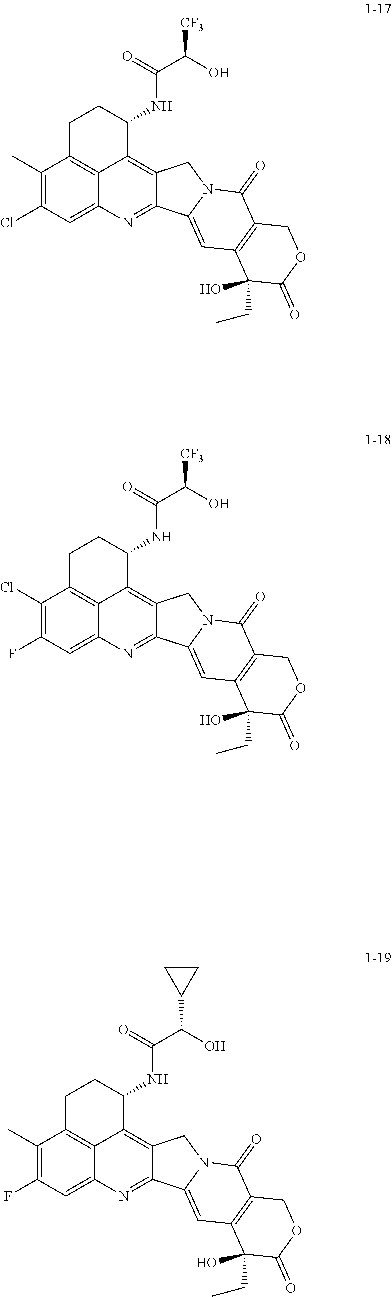 [SMILES: CC[C@@]1(O)C(=O)OCc2c1cc1n(c2=O)Cc2c-1nc1cc(Cl)c(C)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(C)c3c1c2[C@@H](NC(=O)[C@@H](O)C1CC1)CC3]

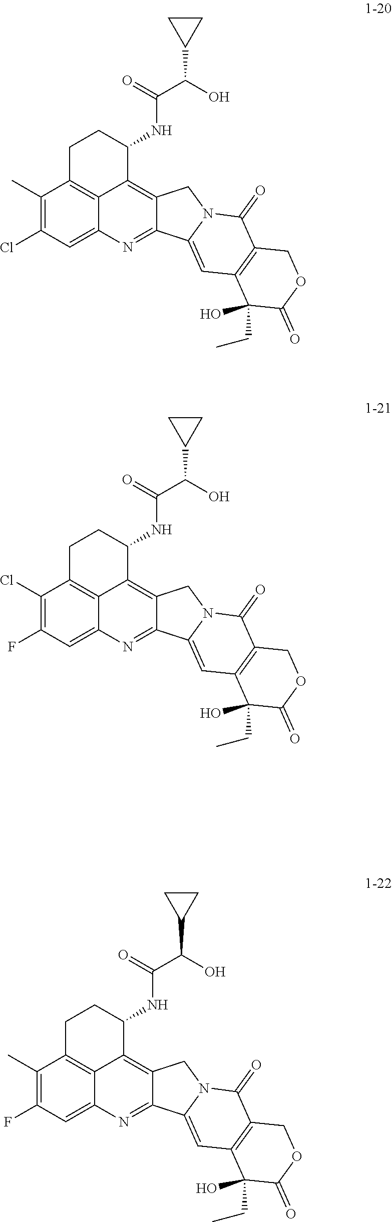 [SMILES: CC[C@@]1(O)C(=O)OCc2c1cc1n(c2=O)Cc2c-1nc1cc(Cl)c(C)c3c1c2[C@@H](NC(=O)[C@@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(F)c(C)c3c1c2[C@@H](NC(=O)[C@H](O)C1CC1)CC3]

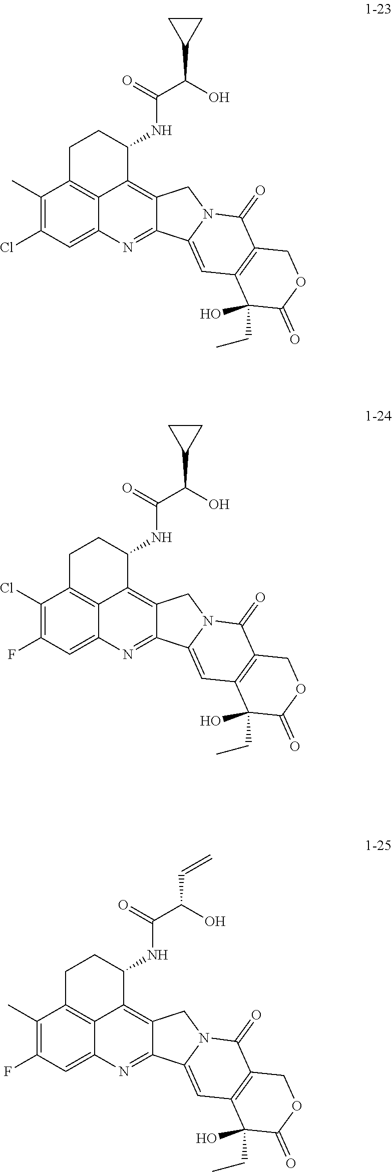 [SMILES: CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3 ; C=C[C@H](O)C(=O)N[C@H]1CCc2c(C)c(F)cc3nc4c(c1c23)Cn1c-4cc2c(c1=O)COC(=O)[C@]2(O)CC]

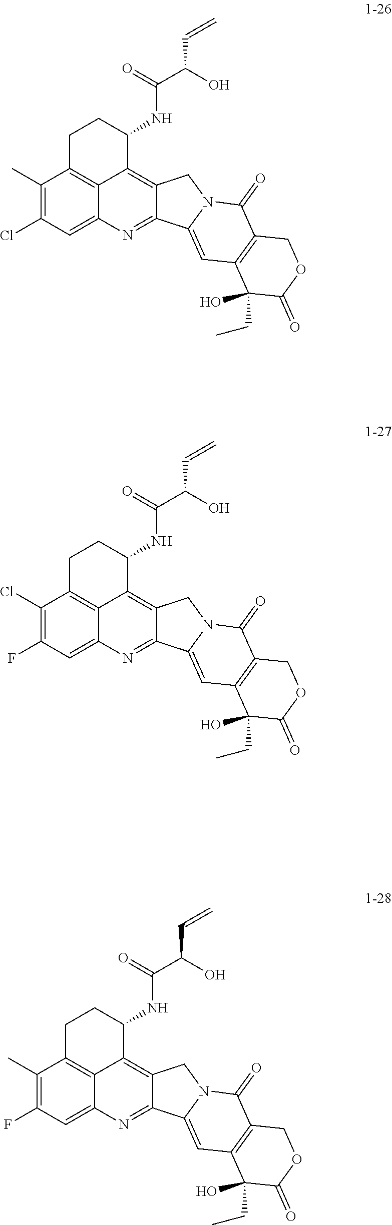 [SMILES: C=C[C@H](O)C(=O)N[C@H]1CCc2c(C)c(Cl)cc3nc4c(c1c23)Cn1c-4cc2c(c1=O)COC(=O)[C@]2(O)CC ; C=C[C@H](O)C(=O)N[C@H]1CCc2c(Cl)c(F)cc3nc4c(c1c23)Cn1c-4cc2c(c1=O)COC(=O)[C@]2(O)CC ; C=C[C@@H](O)C(=O)N[C@H]1CCc2c(C)c(F)cc3nc4c(c1c23)Cn1c-4cc2c(c1=O)COC(=O)[C@]2(O)CC]

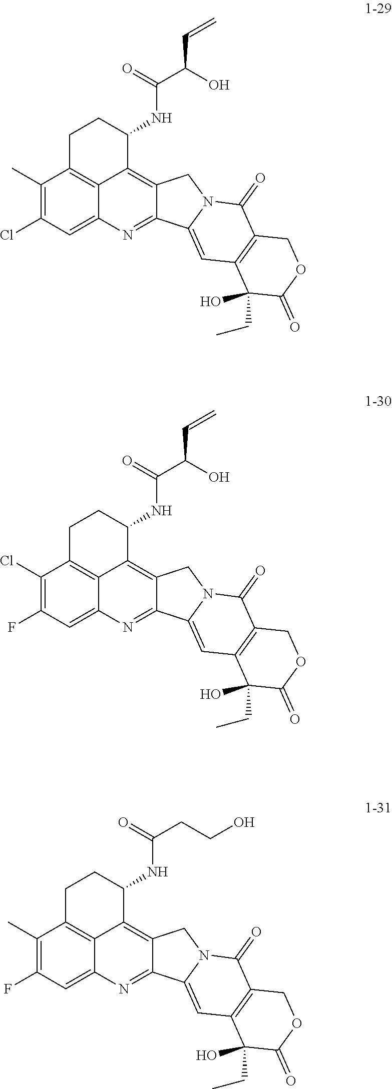 [SMILES: C=C[C@@H](O)C(=O)N[C@H]1CCc2c(C)c(Cl)cc3nc4c(c1c23)Cn1c-4cc2c(c1=O)COC(=O)[C@]2(O)CC ; C=C[C@@H](O)C(=O)N[C@H]1CCc2c(Cl)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)CCO)CC3]

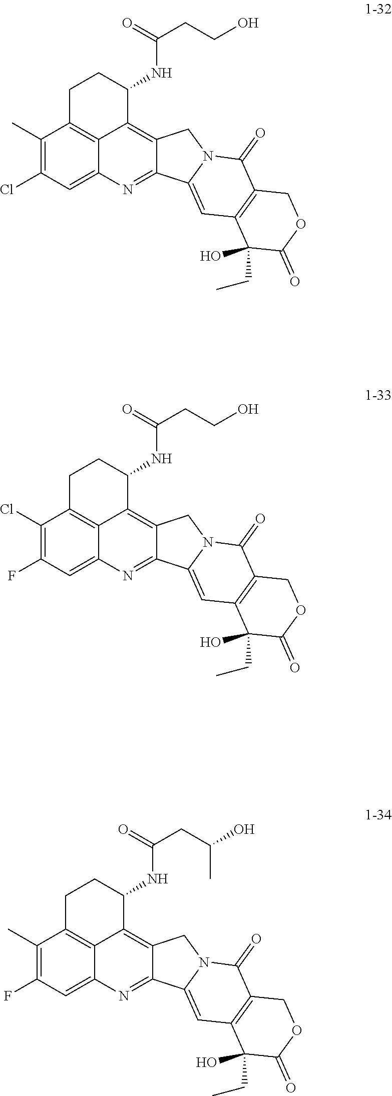 [SMILES: CC[C@@]1(O)C(=O)OCc2c1cc1n(c2=O)Cc2c-1nc1cc(Cl)c(C)c3c1c2[C@@H](NC(=O)CCO)CC3 ; CC[C@@]1(O)C(=O)OCc2c1cc1n(c2=O)Cc2c-1nc1cc(F)c(Cl)c3c1c2[C@@H](NC(=O)CCO)CC3 ; CC[C@@]1(O)C(=O)OCc2c1cc1n(c2=O)Cc2c-1nc1cc(F)c(C)c3c1c2[C@@H](NC(=O)C[C@@H](C)O)CC3]

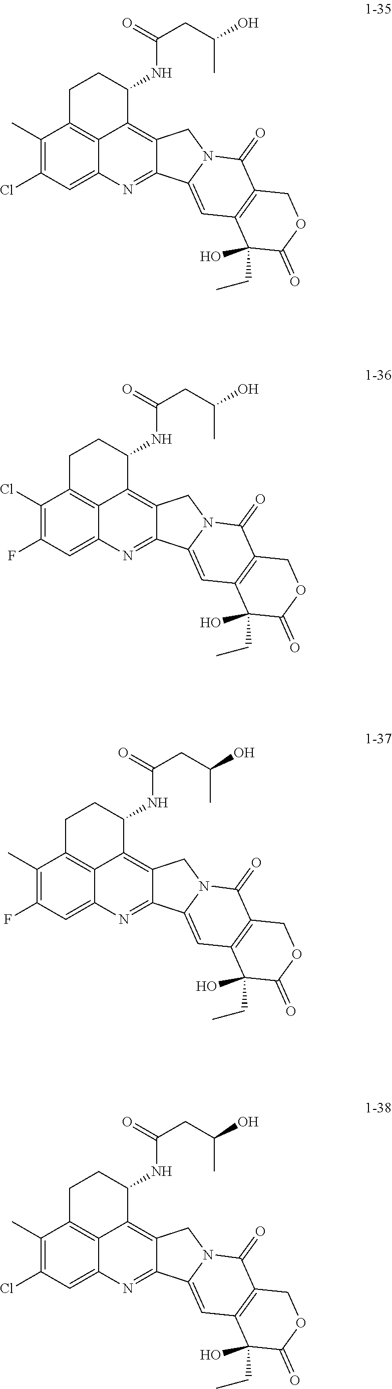 [SMILES: CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(C)c3c1c2[C@@H](NC(=O)C[C@H](C)O)CC3 ; CC[C@@]1(O)C(=O)OCc2c1cc1n(c2=O)Cc2c-1nc1cc(Cl)c(C)c3c1c2[C@@H](NC(=O)C[C@H](C)O)CC3]

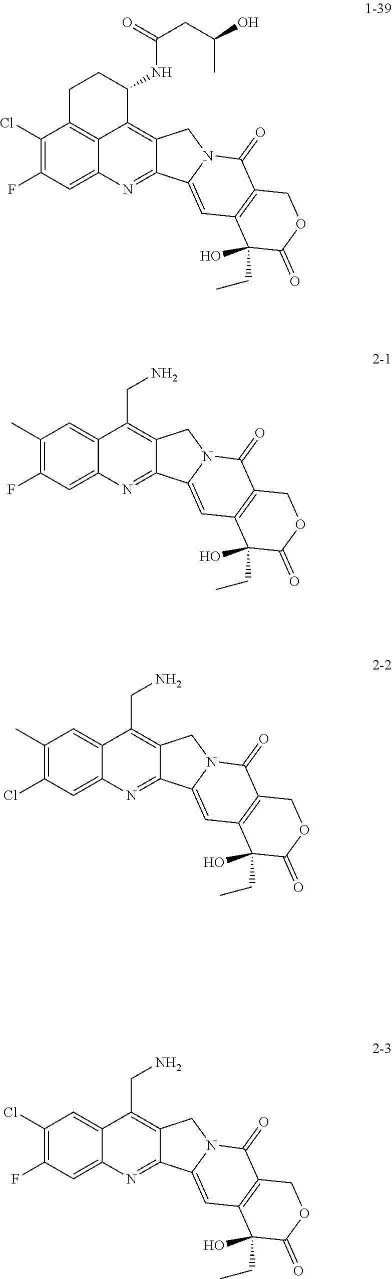 [SMILES: CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(F)c(C)cc1c2CN ; CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN]

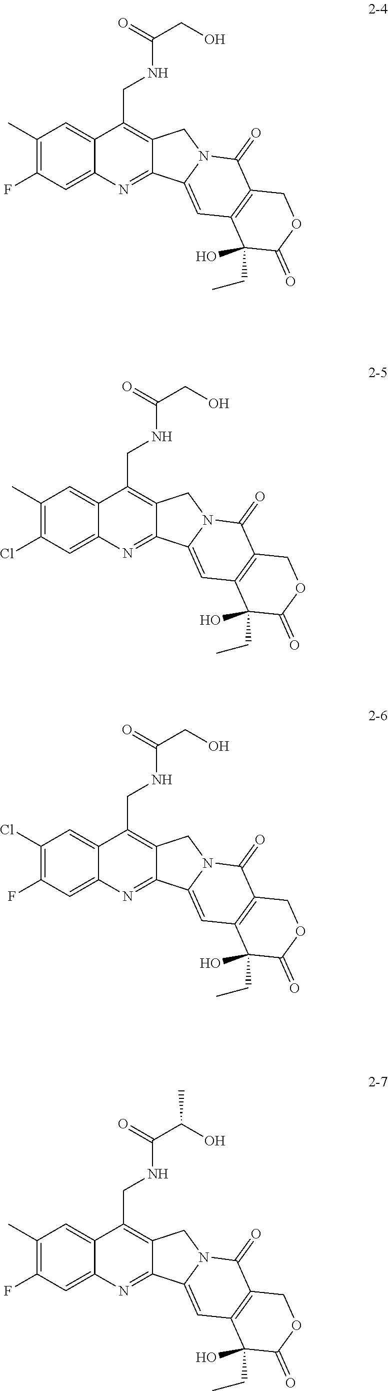 [SMILES: CC[C@@]1(O)C(=O)OCc2c1cc1n(c2=O)Cc2c-1nc1cc(F)c(C)cc1c2CNC(=O)CO ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(F)c(C)cc1c2CNC(=O)[C@H](C)O]

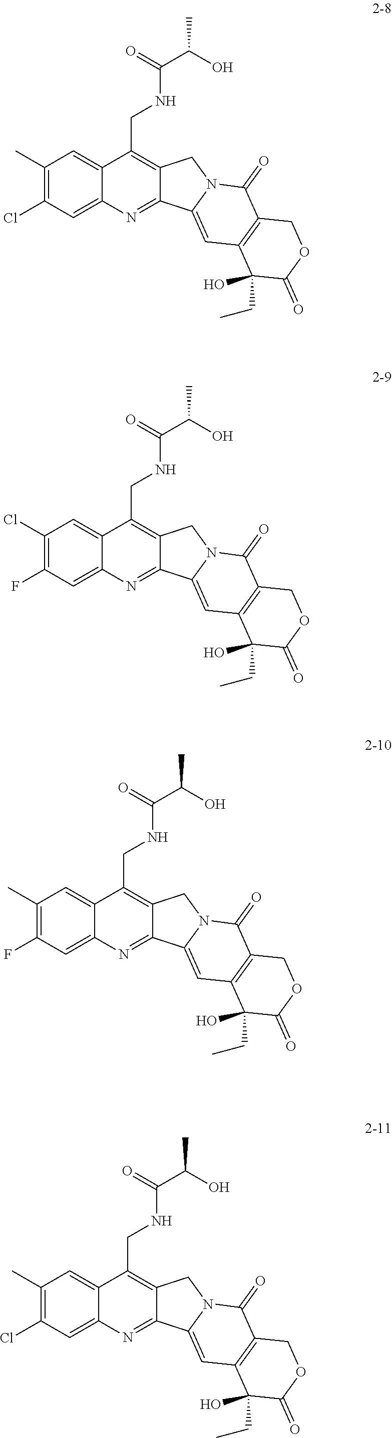 [SMILES: CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(F)c(C)cc1c2CNC(=O)[C@@H](C)O ; CC[C@@]1(O)C(=O)OCc2c1cc1n(c2=O)Cc2c-1nc1cc(Cl)c(C)cc1c2CNC(=O)[C@@H](C)O]

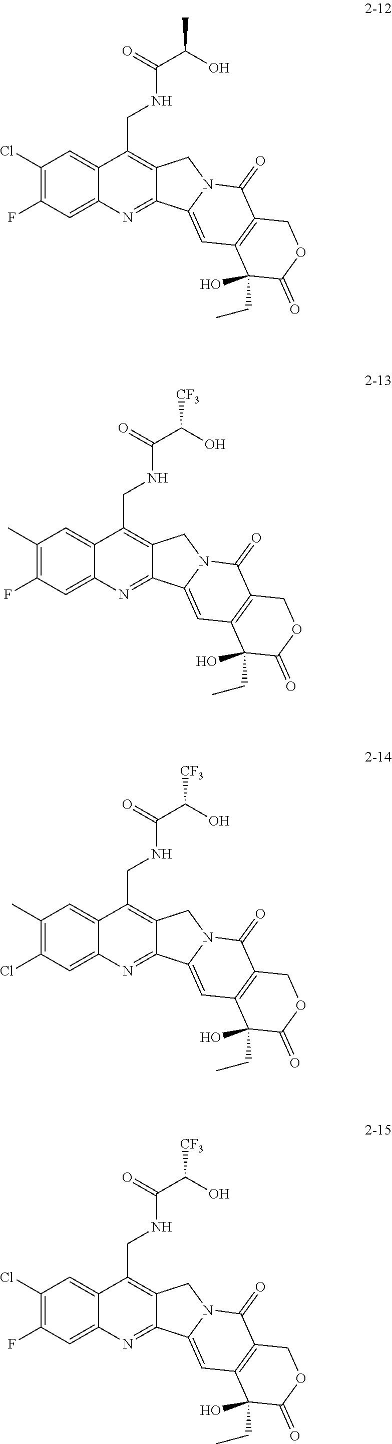 [SMILES: CC[C@@]1(O)C(=O)OCc2c1cc1n(c2=O)Cc2c-1nc1cc(F)c(Cl)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](O)C(F)(F)F ; CC[C@@]1(O)C(=O)OCc2c1cc1n(c2=O)Cc2c-1nc1cc(Cl)c(C)cc1c2CNC(=O)[C@@H](O)C(F)(F)F ; CC[C@@]1(O)C(=O)OCc2c1cc1n(c2=O)Cc2c-1nc1cc(F)c(Cl)cc1c2CNC(=O)[C@@H](O)C(F)(F)F]

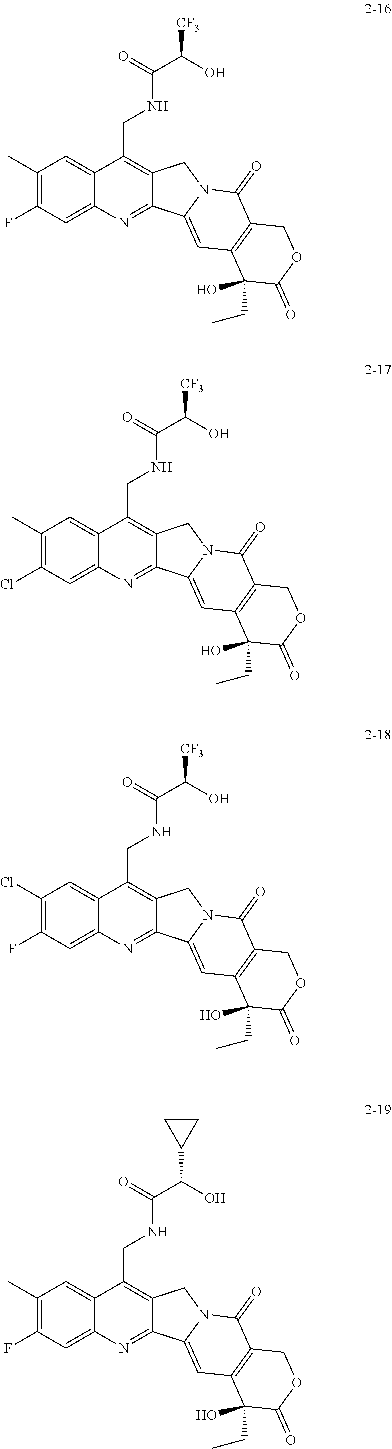 [SMILES: CC[C@@]1(O)C(=O)OCc2c1cc1n(c2=O)Cc2c-1nc1cc(F)c(C)cc1c2CNC(=O)[C@H](O)C(F)(F)F ; CC[C@@]1(O)C(=O)OCc2c1cc1n(c2=O)Cc2c-1nc1cc(Cl)c(C)cc1c2CNC(=O)[C@H](O)C(F)(F)F ; CC[C@@]1(O)C(=O)OCc2c1cc1n(c2=O)Cc2c-1nc1cc(F)c(Cl)cc1c2CNC(=O)[C@H](O)C(F)(F)F ; CC[C@@]1(O)C(=O)OCc2c1cc1n(c2=O)Cc2c-1nc1cc(F)c(C)cc1c2CNC(=O)[C@@H](O)C1CC1]

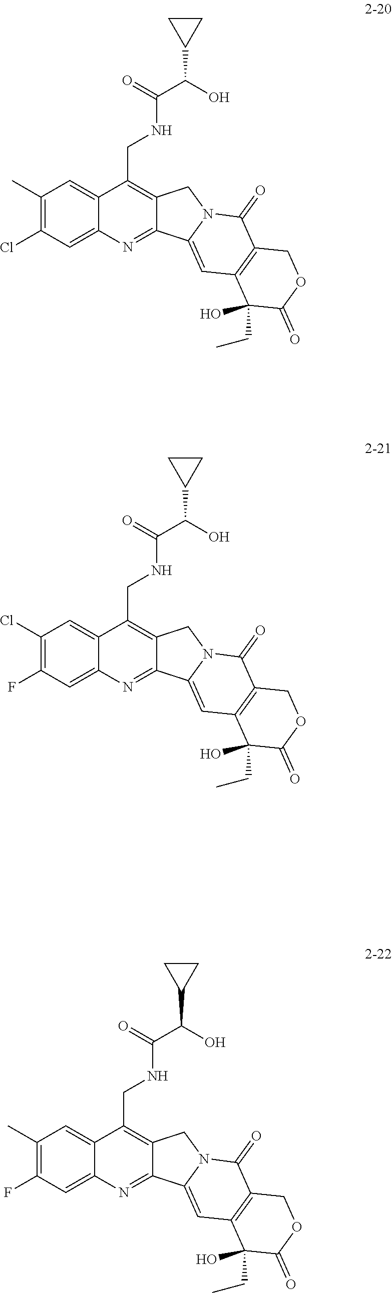 [SMILES: CC[C@@]1(O)C(=O)OCc2c1cc1n(c2=O)Cc2c-1nc1cc(Cl)c(C)cc1c2CNC(=O)[C@@H](O)C1CC1 ; CC[C@@]1(O)C(=O)OCc2c1cc1n(c2=O)Cc2c-1nc1cc(F)c(Cl)cc1c2CNC(=O)[C@@H](O)C1CC1 ; CC[C@@]1(O)C(=O)OCc2c1cc1n(c2=O)Cc2c-1nc1cc(F)c(C)cc1c2CNC(=O)[C@H](O)C1CC1]

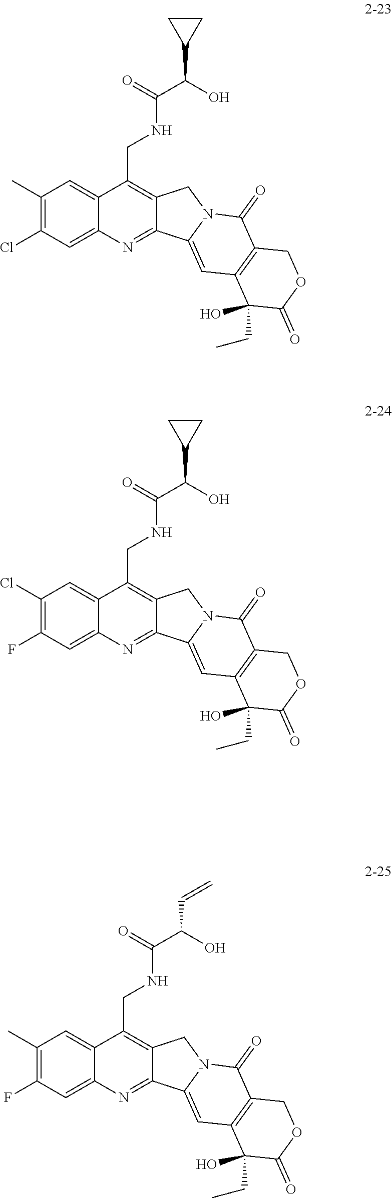 [SMILES: CC[C@@]1(O)C(=O)OCc2c1cc1n(c2=O)Cc2c-1nc1cc(Cl)c(C)cc1c2CNC(=O)[C@H](O)C1CC1 ; CC[C@@]1(O)C(=O)OCc2c1cc1n(c2=O)Cc2c-1nc1cc(F)c(Cl)cc1c2CNC(=O)[C@H](O)C1CC1 ; C=C[C@H](O)C(=O)NCc1c2c(nc3cc(F)c(C)cc13)-c1cc3c(c(=O)n1C2)COC(=O)[C@]3(O)CC]

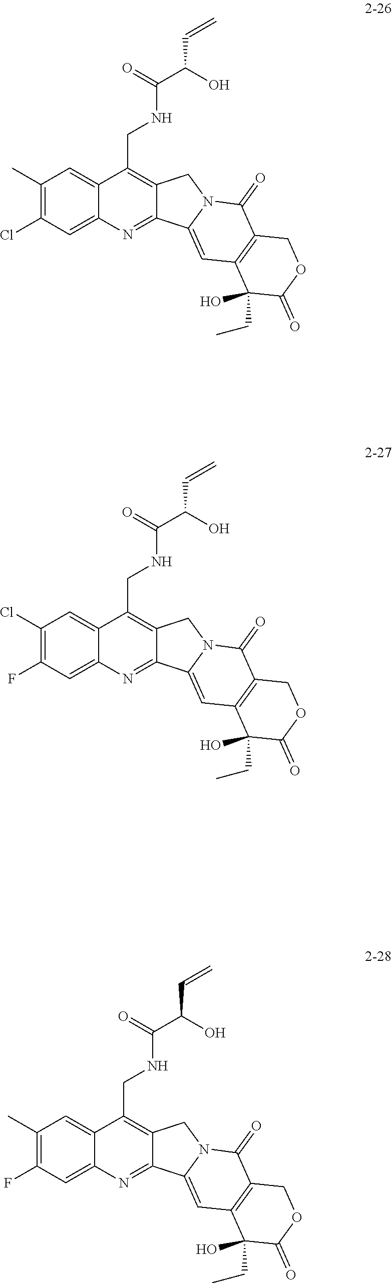 [SMILES: C=C[C@H](O)C(=O)NCc1c2c(nc3cc(Cl)c(C)cc13)-c1cc3c(c(=O)n1C2)COC(=O)[C@]3(O)CC ; C=C[C@H](O)C(=O)NCc1c2c(nc3cc(F)c(Cl)cc13)-c1cc3c(c(=O)n1C2)COC(=O)[C@]3(O)CC ; C=C[C@@H](O)C(=O)NCc1c2c(nc3cc(F)c(C)cc13)-c1cc3c(c(=O)n1C2)COC(=O)[C@]3(O)CC]

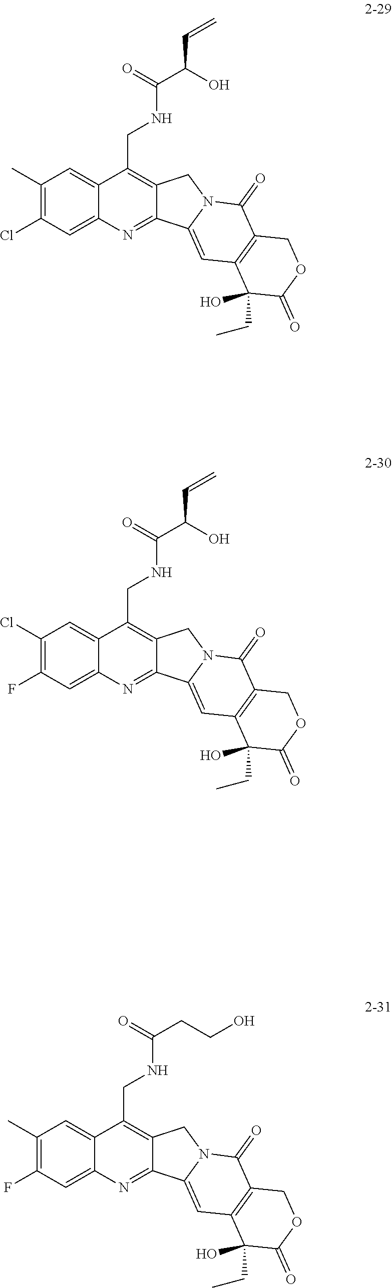 [SMILES: C=C[C@@H](O)C(=O)NCc1c2c(nc3cc(Cl)c(C)cc13)-c1cc3c(c(=O)n1C2)COC(=O)[C@]3(O)CC ; C=C[C@@H](O)C(=O)NCc1c2c(nc3cc(F)c(Cl)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2CNC(=O)CCO]

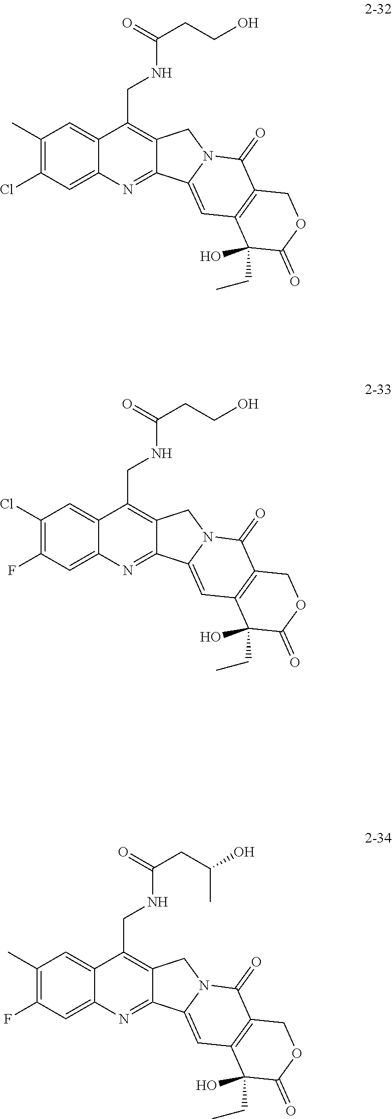 [SMILES: CC[C@@]1(O)C(=O)OCc2c1cc1n(c2=O)Cc2c-1nc1cc(Cl)c(C)cc1c2CNC(=O)CCO ; CC[C@@]1(O)C(=O)OCc2c1cc1n(c2=O)Cc2c-1nc1cc(F)c(Cl)cc1c2CNC(=O)CCO ; CC[C@@]1(O)C(=O)OCc2c1cc1n(c2=O)Cc2c-1nc1cc(F)c(C)cc1c2CNC(=O)C[C@@H](C)O]

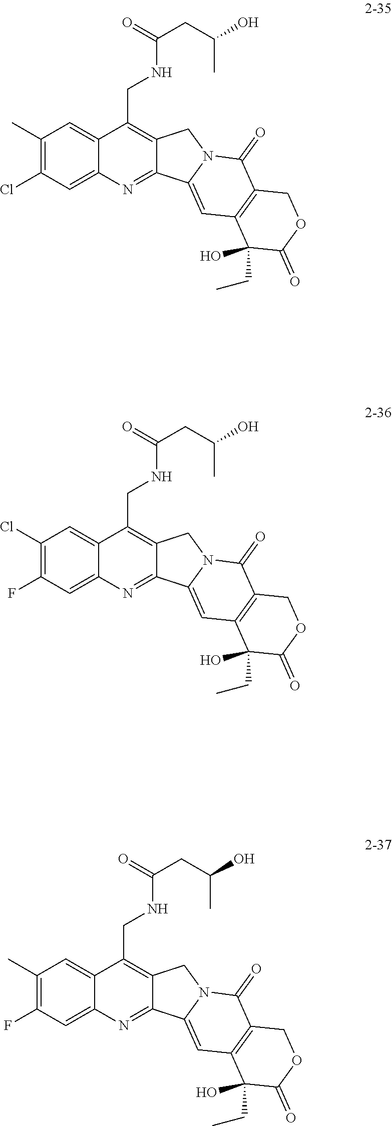 [SMILES: CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O]

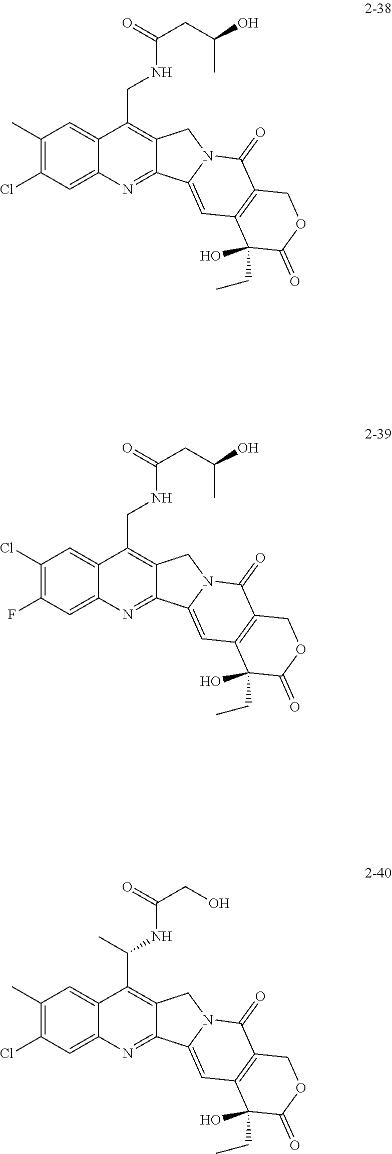 [SMILES: CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2[C@H](C)NC(=O)CO]

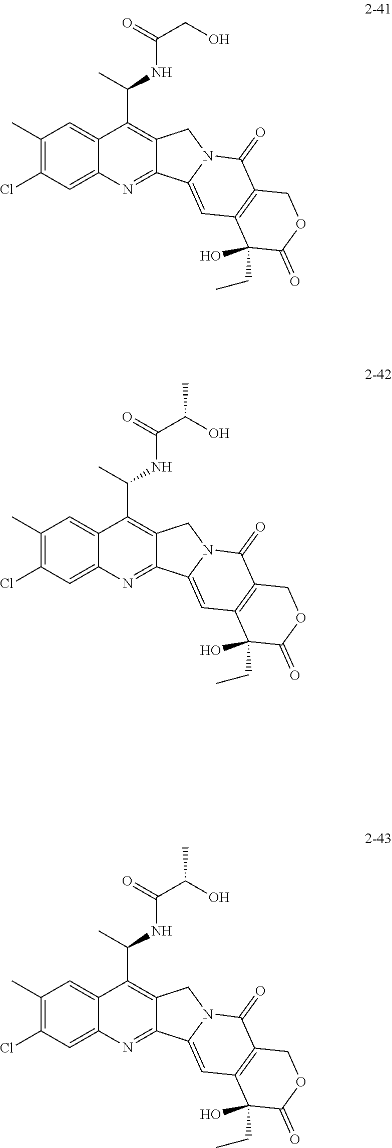 [SMILES: CC[C@@]1(O)C(=O)OCc2c1cc1n(c2=O)Cc2c-1nc1cc(Cl)c(C)cc1c2[C@@H](C)NC(=O)CO ; CC[C@@]1(O)C(=O)OCc2c1cc1n(c2=O)Cc2c-1nc1cc(Cl)c(C)cc1c2[C@H](C)NC(=O)[C@H](C)O ; CC[C@@]1(O)C(=O)OCc2c1cc1n(c2=O)Cc2c-1nc1cc(Cl)c(C)cc1c2[C@@H](C)NC(=O)[C@H](C)O]

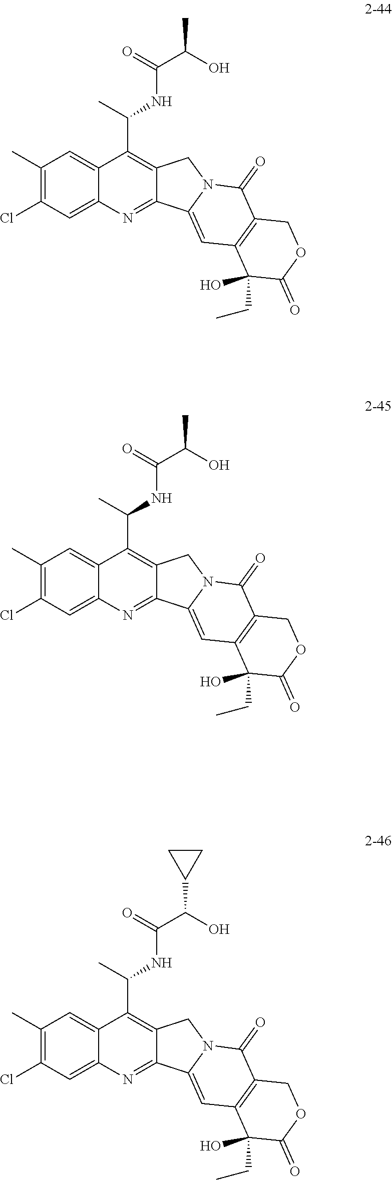 [SMILES: CC[C@@]1(O)C(=O)OCc2c1cc1n(c2=O)Cc2c-1nc1cc(Cl)c(C)cc1c2[C@H](C)NC(=O)[C@@H](C)O ; CC[C@@]1(O)C(=O)OCc2c1cc1n(c2=O)Cc2c-1nc1cc(Cl)c(C)cc1c2[C@@H](C)NC(=O)[C@@H](C)O ; CC[C@@]1(O)C(=O)OCc2c1cc1n(c2=O)Cc2c-1nc1cc(Cl)c(C)cc1c2[C@H](C)NC(=O)[C@@H](O)C1CC1]

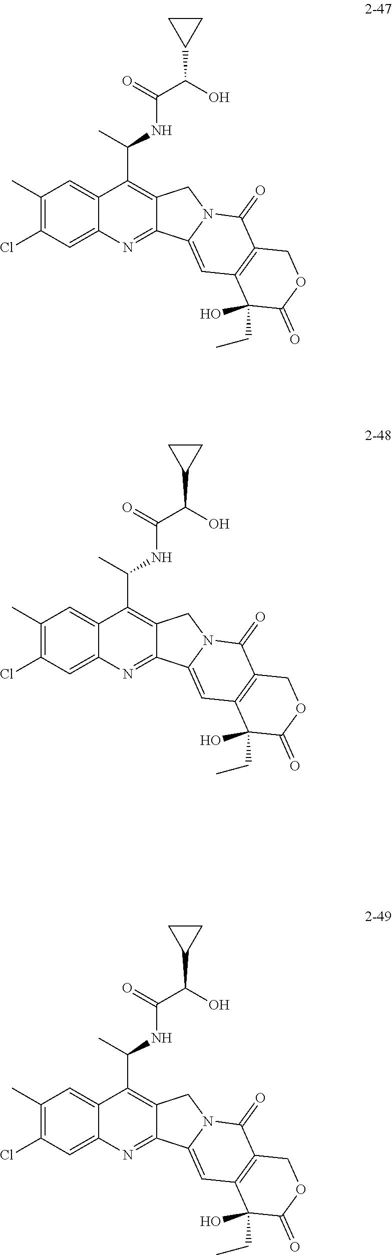 [SMILES: CC[C@@]1(O)C(=O)OCc2c1cc1n(c2=O)Cc2c-1nc1cc(Cl)c(C)cc1c2[C@@H](C)NC(=O)[C@@H](O)C1CC1 ; CC[C@@]1(O)C(=O)OCc2c1cc1n(c2=O)Cc2c-1nc1cc(Cl)c(C)cc1c2[C@H](C)NC(=O)[C@H](O)C1CC1 ; CC[C@@]1(O)C(=O)OCc2c1cc1n(c2=O)Cc2c-1nc1cc(Cl)c(C)cc1c2[C@@H](C)NC(=O)[C@H](O)C1CC1]

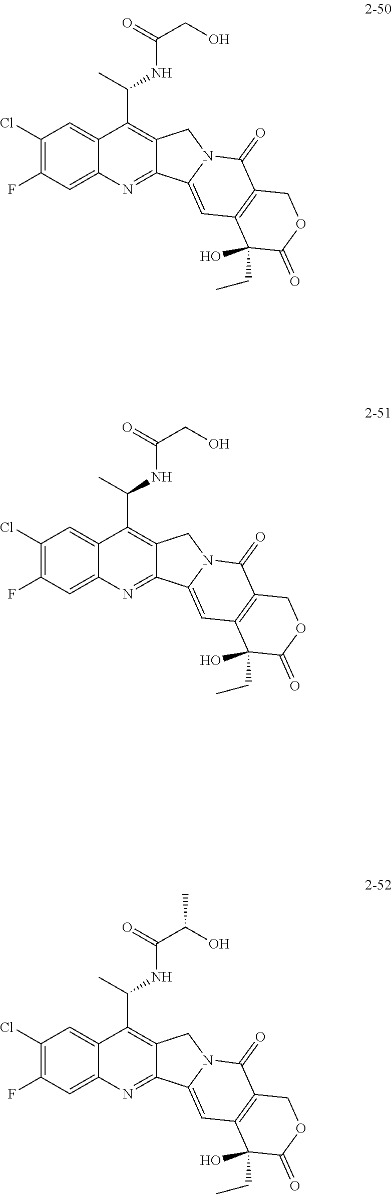 [SMILES: CC[C@@]1(O)C(=O)OCc2c1cc1n(c2=O)Cc2c-1nc1cc(F)c(Cl)cc1c2[C@H](C)NC(=O)CO ; CC[C@@]1(O)C(=O)OCc2c1cc1n(c2=O)Cc2c-1nc1cc(F)c(Cl)cc1c2[C@@H](C)NC(=O)CO ; CC[C@@]1(O)C(=O)OCc2c1cc1n(c2=O)Cc2c-1nc1cc(F)c(Cl)cc1c2[C@H](C)NC(=O)[C@H](C)O]

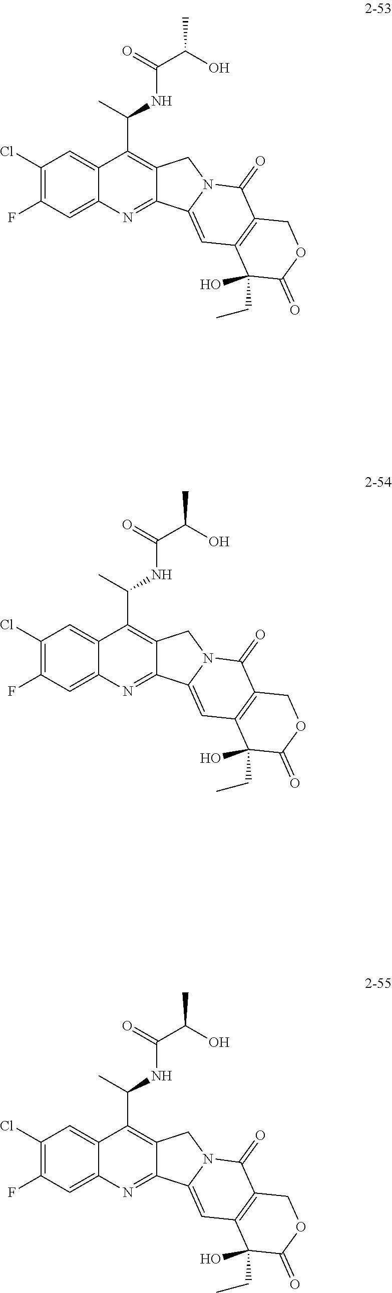 [SMILES: CC[C@@]1(O)C(=O)OCc2c1cc1n(c2=O)Cc2c-1nc1cc(F)c(Cl)cc1c2[C@@H](C)NC(=O)[C@H](C)O ; CC[C@@]1(O)C(=O)OCc2c1cc1n(c2=O)Cc2c-1nc1cc(F)c(Cl)cc1c2[C@H](C)NC(=O)[C@@H](C)O ; CC[C@@]1(O)C(=O)OCc2c1cc1n(c2=O)Cc2c-1nc1cc(F)c(Cl)cc1c2[C@@H](C)NC(=O)[C@@H](C)O]

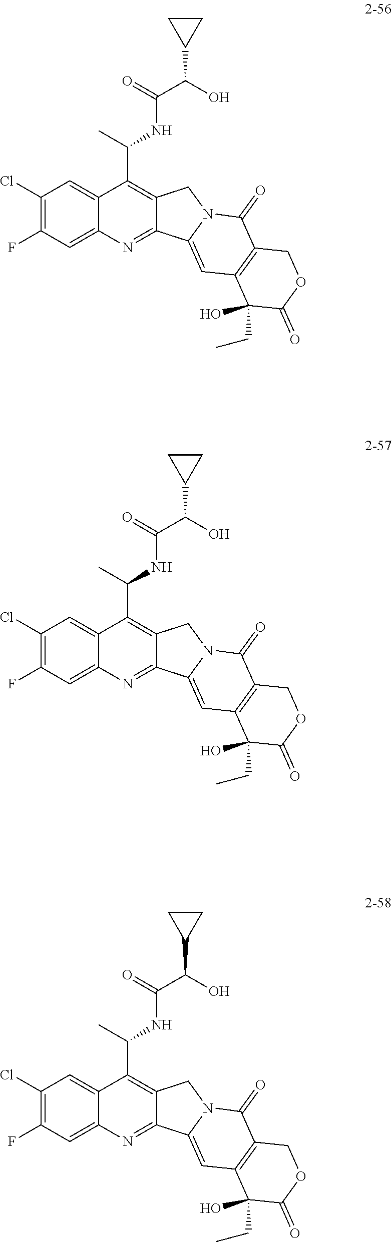 [SMILES: CC[C@@]1(O)C(=O)OCc2c1cc1n(c2=O)Cc2c-1nc1cc(F)c(Cl)cc1c2[C@H](C)NC(=O)[C@@H](O)C1CC1 ; CC[C@@]1(O)C(=O)OCc2c1cc1n(c2=O)Cc2c-1nc1cc(F)c(Cl)cc1c2[C@@H](C)NC(=O)[C@@H](O)C1CC1 ; CC[C@@]1(O)C(=O)OCc2c1cc1n(c2=O)Cc2c-1nc1cc(F)c(Cl)cc1c2[C@H](C)NC(=O)[C@H](O)C1CC1]

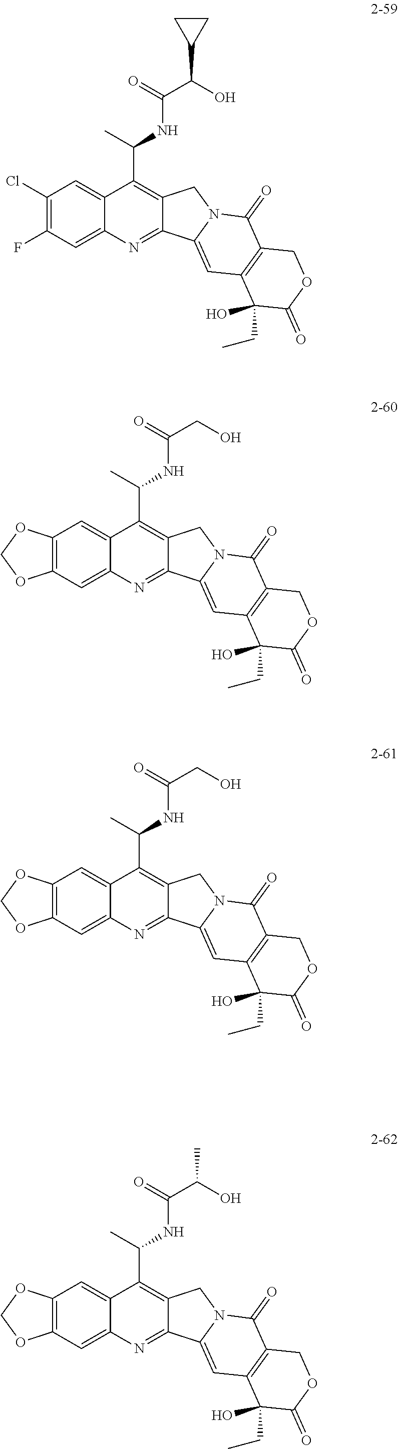 [SMILES: CC[C@@]1(O)C(=O)OCc2c1cc1n(c2=O)Cc2c-1nc1cc(F)c(Cl)cc1c2[C@@H](C)NC(=O)[C@H](O)C1CC1 ; CC[C@@]1(O)C(=O)OCc2c1cc1n(c2=O)Cc2c-1nc1cc3c(cc1c2[C@H](C)NC(=O)CO)OCO3 ; CC[C@@]1(O)C(=O)OCc2c1cc1n(c2=O)Cc2c-1nc1cc3c(cc1c2[C@@H](C)NC(=O)CO)OCO3 ; CC[C@@]1(O)C(=O)OCc2c1cc1n(c2=O)Cc2c-1nc1cc3c(cc1c2[C@H](C)NC(=O)[C@H](C)O)OCO3]

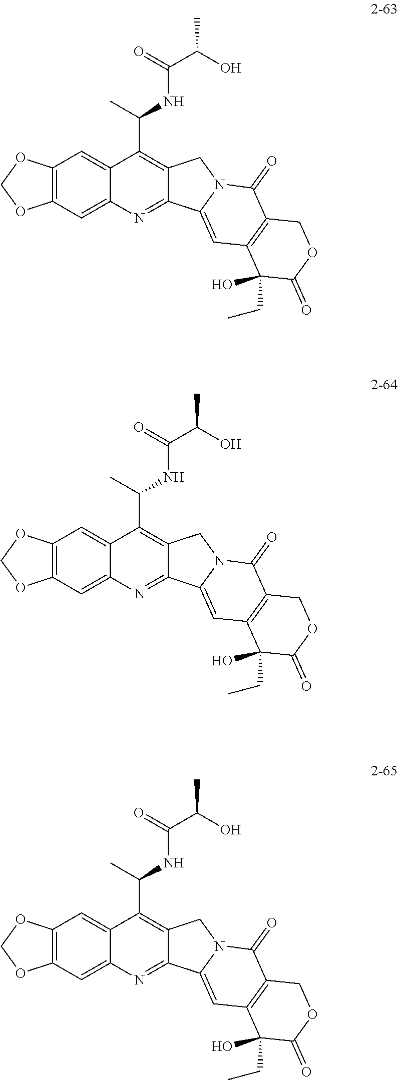 [SMILES: CC[C@@]1(O)C(=O)OCc2c1cc1n(c2=O)Cc2c-1nc1cc3c(cc1c2[C@@H](C)NC(=O)[C@H](C)O)OCO3 ; CC[C@@]1(O)C(=O)OCc2c1cc1n(c2=O)Cc2c-1nc1cc3c(cc1c2[C@H](C)NC(=O)[C@@H](C)O)OCO3 ; CC[C@@]1(O)C(=O)OCc2c1cc1n(c2=O)Cc2c-1nc1cc3c(cc1c2[C@@H](C)NC(=O)[C@@H](C)O)OCO3]

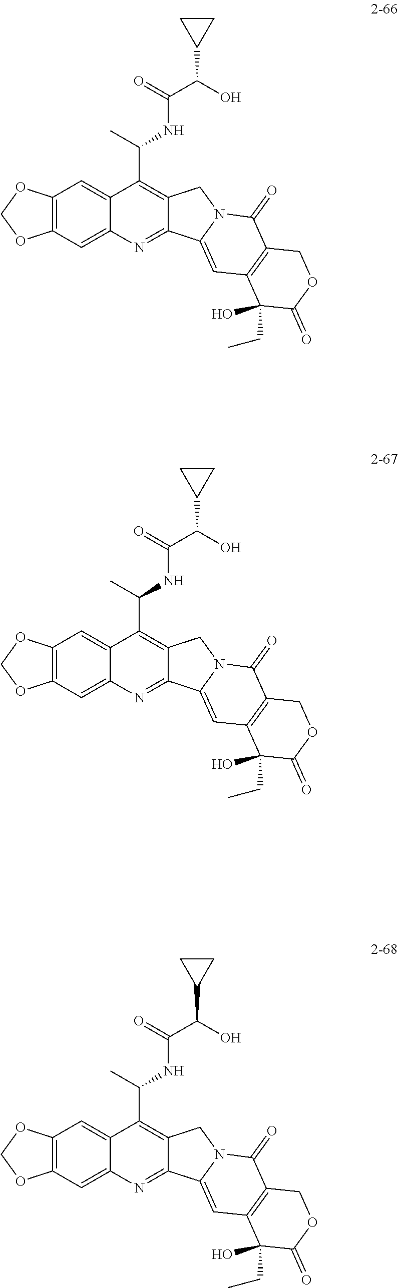 [SMILES: CC[C@@]1(O)C(=O)OCc2c1cc1n(c2=O)Cc2c-1nc1cc3c(cc1c2[C@H](C)NC(=O)[C@@H](O)C1CC1)OCO3 ; CC[C@@]1(O)C(=O)OCc2c1cc1n(c2=O)Cc2c-1nc1cc3c(cc1c2[C@@H](C)NC(=O)[C@@H](O)C1CC1)OCO3 ; CC[C@@]1(O)C(=O)OCc2c1cc1n(c2=O)Cc2c-1nc1cc3c(cc1c2[C@H](C)NC(=O)[C@H](O)C1CC1)OCO3]

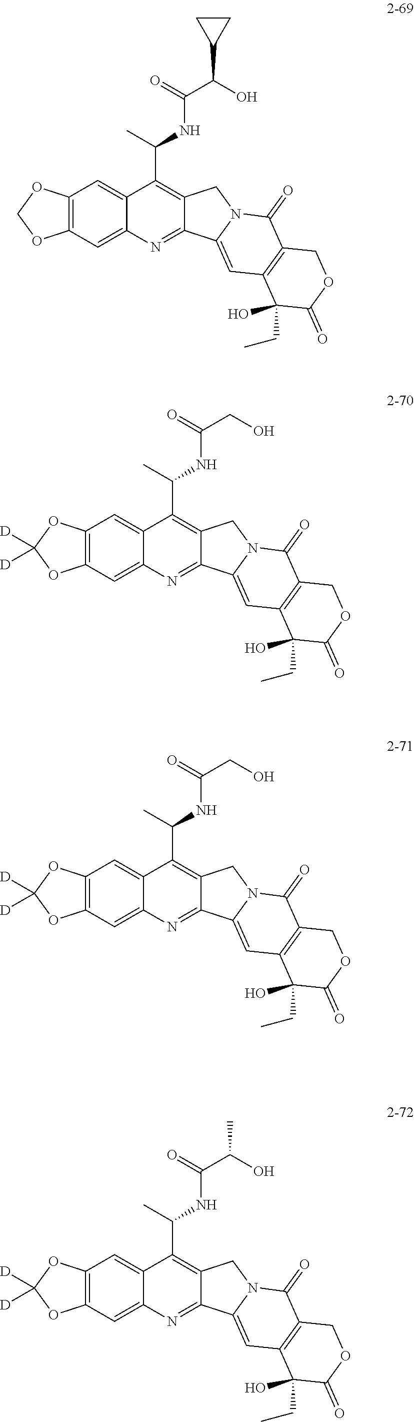 [SMILES: CC[C@@]1(O)C(=O)OCc2c1cc1n(c2=O)Cc2c-1nc1cc3c(cc1c2[C@@H](C)NC(=O)[C@H](O)C1CC1)OCO3 ; [2H]C1([2H])Oc2cc3nc4c(c([C@H](C)NC(=O)CO)c3cc2O1)Cn1c-4cc2c(c1=O)COC(=O)[C@]2(O)CC ; [2H]C1([2H])Oc2cc3nc4c(c([C@@H](C)NC(=O)CO)c3cc2O1)Cn1c-4cc2c(c1=O)COC(=O)[C@]2(O)CC ; [2H]C1([2H])Oc2cc3nc4c(c([C@H](C)NC(=O)[C@H](C)O)c3cc2O1)Cn1c-4cc2c(c1=O)COC(=O)[C@]2(O)CC]

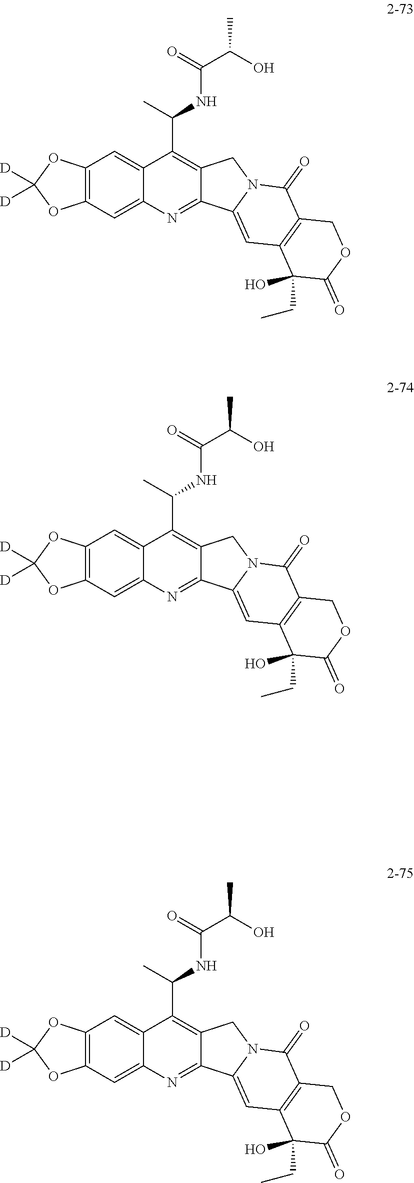 [SMILES: [2H]C1([2H])Oc2cc3nc4c(c([C@@H](C)NC(=O)[C@H](C)O)c3cc2O1)Cn1c-4cc2c(c1=O)COC(=O)[C@]2(O)CC ; [2H]C1([2H])Oc2cc3nc4c(c([C@H](C)NC(=O)[C@@H](C)O)c3cc2O1)Cn1c-4cc2c(c1=O)COC(=O)[C@]2(O)CC ; [2H]C1([2H])Oc2cc3nc4c(c([C@@H](C)NC(=O)[C@@H](C)O)c3cc2O1)Cn1c-4cc2c(c1=O)COC(=O)[C@]2(O)CC]

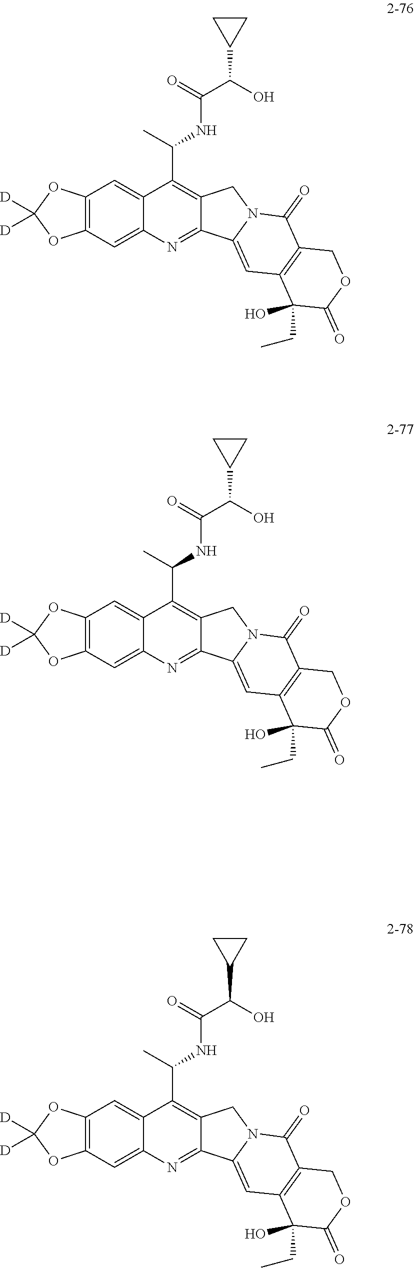 [SMILES: [2H]C1([2H])Oc2cc3nc4c(c([C@H](C)NC(=O)[C@@H](O)C5CC5)c3cc2O1)Cn1c-4cc2c(c1=O)COC(=O)[C@]2(O)CC ; [2H]C1([2H])Oc2cc3nc4c(c([C@@H](C)NC(=O)[C@@H](O)C5CC5)c3cc2O1)Cn1c-4cc2c(c1=O)COC(=O)[C@]2(O)CC ; [2H]C1([2H])Oc2cc3nc4c(c([C@H](C)NC(=O)[C@H](O)C5CC5)c3cc2O1)Cn1c-4cc2c(c1=O)COC(=O)[C@]2(O)CC]

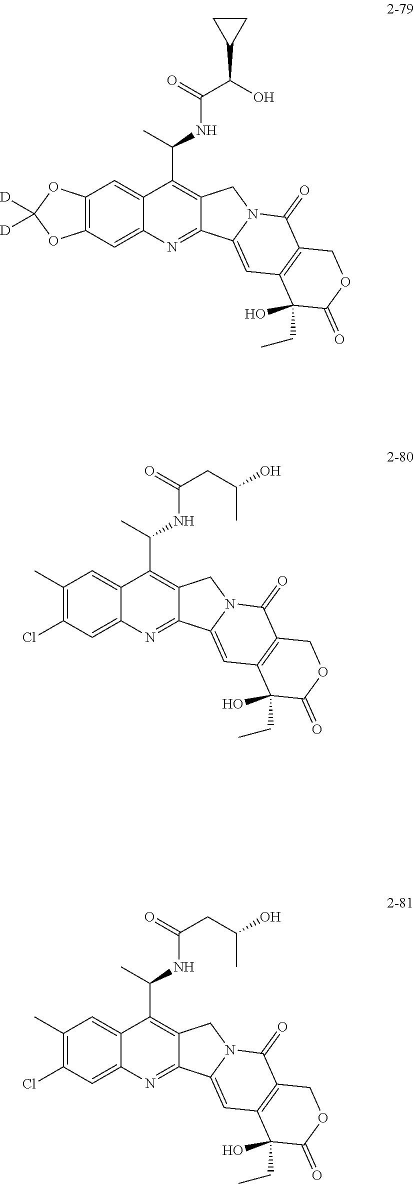 [SMILES: [2H]C1([2H])Oc2cc3nc4c(c([C@@H](C)NC(=O)[C@H](O)C5CC5)c3cc2O1)Cn1c-4cc2c(c1=O)COC(=O)[C@]2(O)CC ; CC[C@@]1(O)C(=O)OCc2c1cc1n(c2=O)Cc2c-1nc1cc(Cl)c(C)cc1c2[C@H](C)NC(=O)C[C@@H](C)O ; CC[C@@]1(O)C(=O)OCc2c1cc1n(c2=O)Cc2c-1nc1cc(Cl)c(C)cc1c2[C@@H](C)NC(=O)C[C@@H](C)O]

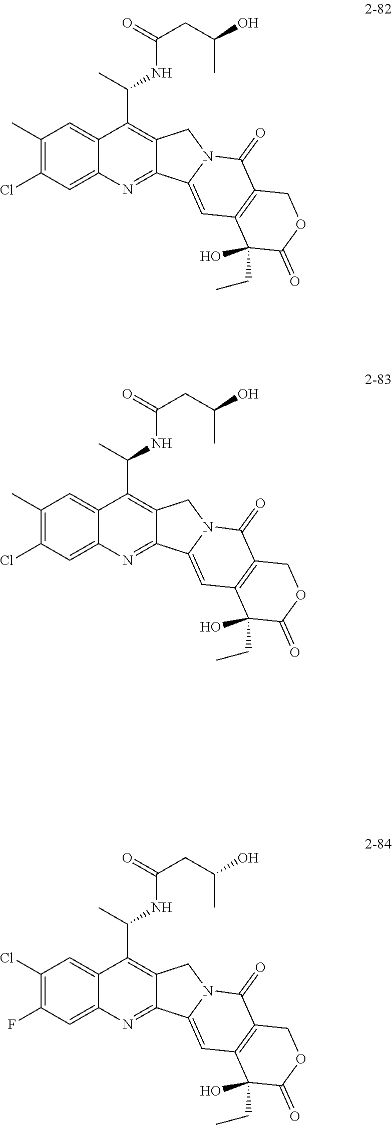 [SMILES: CC[C@@]1(O)C(=O)OCc2c1cc1n(c2=O)Cc2c-1nc1cc(Cl)c(C)cc1c2[C@H](C)NC(=O)C[C@H](C)O ; CC[C@@]1(O)C(=O)OCc2c1cc1n(c2=O)Cc2c-1nc1cc(Cl)c(C)cc1c2[C@@H](C)NC(=O)C[C@H](C)O ; CC[C@@]1(O)C(=O)OCc2c1cc1n(c2=O)Cc2c-1nc1cc(F)c(Cl)cc1c2[C@H](C)NC(=O)C[C@@H](C)O]

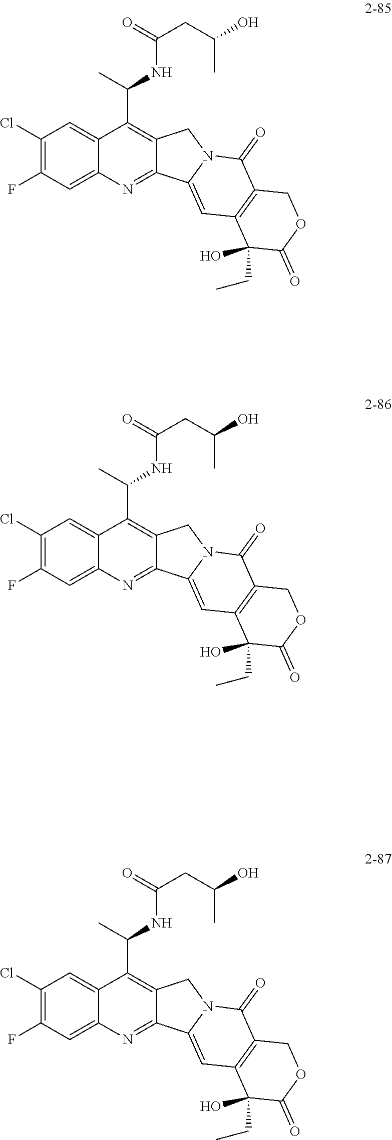 [SMILES: CC[C@@]1(O)C(=O)OCc2c1cc1n(c2=O)Cc2c-1nc1cc(F)c(Cl)cc1c2[C@@H](C)NC(=O)C[C@@H](C)O ; CC[C@@]1(O)C(=O)OCc2c1cc1n(c2=O)Cc2c-1nc1cc(F)c(Cl)cc1c2[C@H](C)NC(=O)C[C@H](C)O ; CC[C@@]1(O)C(=O)OCc2c1cc1n(c2=O)Cc2c-1nc1cc(F)c(Cl)cc1c2[C@@H](C)NC(=O)C[C@H](C)O]

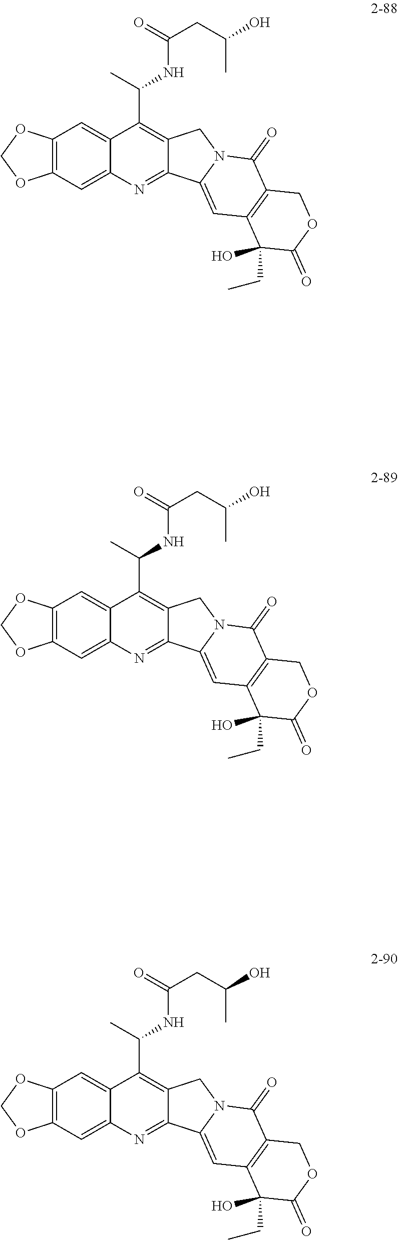 [SMILES: CC[C@@]1(O)C(=O)OCc2c1cc1n(c2=O)Cc2c-1nc1cc3c(cc1c2[C@H](C)NC(=O)C[C@@H](C)O)OCO3 ; CC[C@@]1(O)C(=O)OCc2c1cc1n(c2=O)Cc2c-1nc1cc3c(cc1c2[C@@H](C)NC(=O)C[C@@H](C)O)OCO3 ; CC[C@@]1(O)C(=O)OCc2c1cc1n(c2=O)Cc2c-1nc1cc3c(cc1c2[C@H](C)NC(=O)C[C@H](C)O)OCO3]

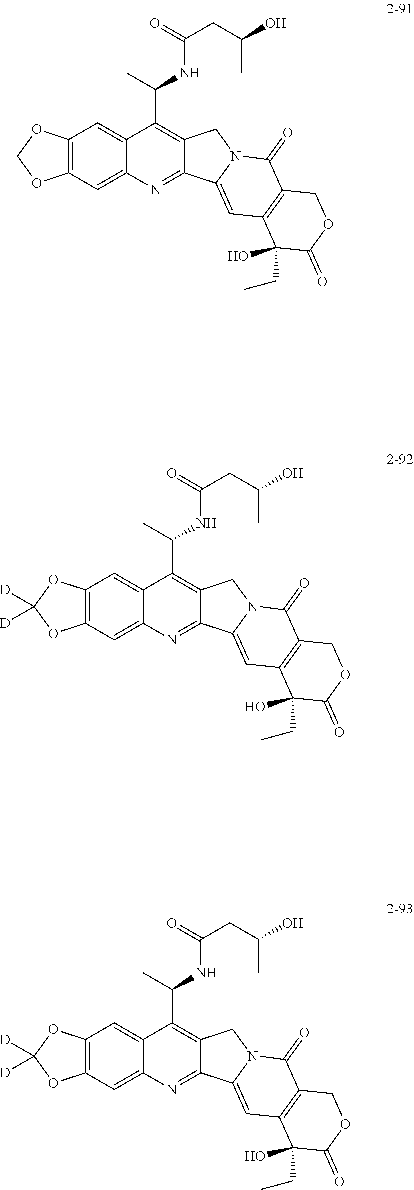 [SMILES: CC[C@@]1(O)C(=O)OCc2c1cc1n(c2=O)Cc2c-1nc1cc3c(cc1c2[C@@H](C)NC(=O)C[C@H](C)O)OCO3 ; [2H]C1([2H])Oc2cc3nc4c(c([C@H](C)NC(=O)C[C@@H](C)O)c3cc2O1)Cn1c-4cc2c(c1=O)COC(=O)[C@]2(O)CC ; [2H]C1([2H])Oc2cc3nc4c(c([C@@H](C)NC(=O)C[C@@H](C)O)c3cc2O1)Cn1c-4cc2c(c1=O)COC(=O)[C@]2(O)CC]

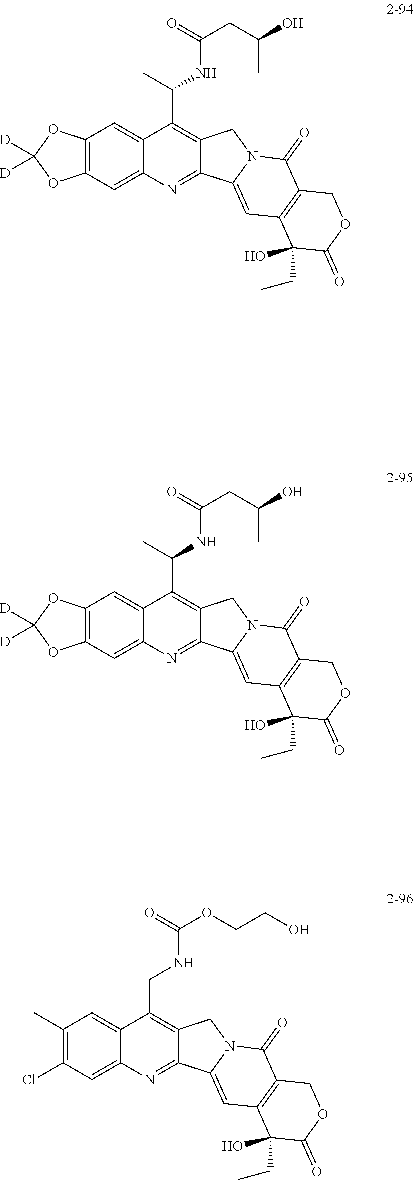 [SMILES: [2H]C1([2H])Oc2cc3nc4c(c([C@H](C)NC(=O)C[C@H](C)O)c3cc2O1)Cn1c-4cc2c(c1=O)COC(=O)[C@]2(O)CC ; [2H]C1([2H])Oc2cc3nc4c(c([C@@H](C)NC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1cc(Cl)c(C)cc1c2CNC(=O)OCCO]

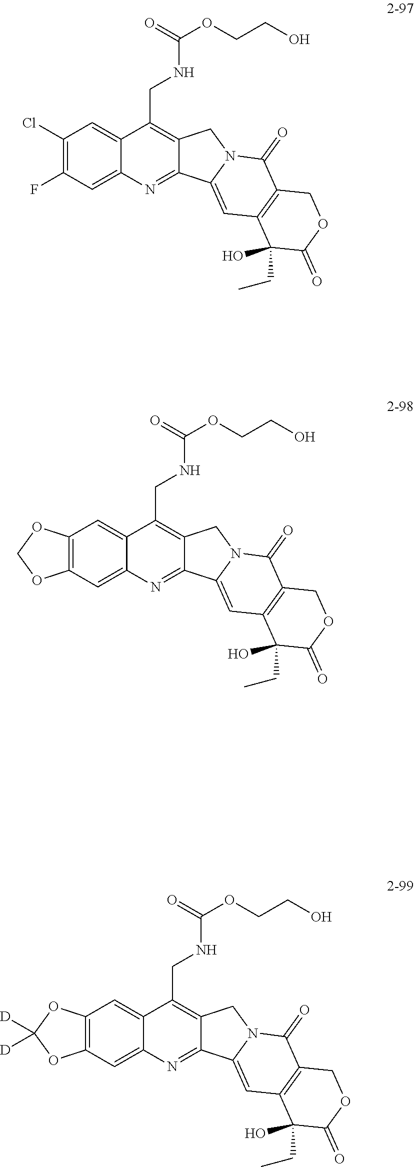 [SMILES: CC[C@@]1(O)C(=O)OCc2c1cc1n(c2=O)Cc2c-1nc1cc(F)c(Cl)cc1c2CNC(=O)OCCO ; CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC]

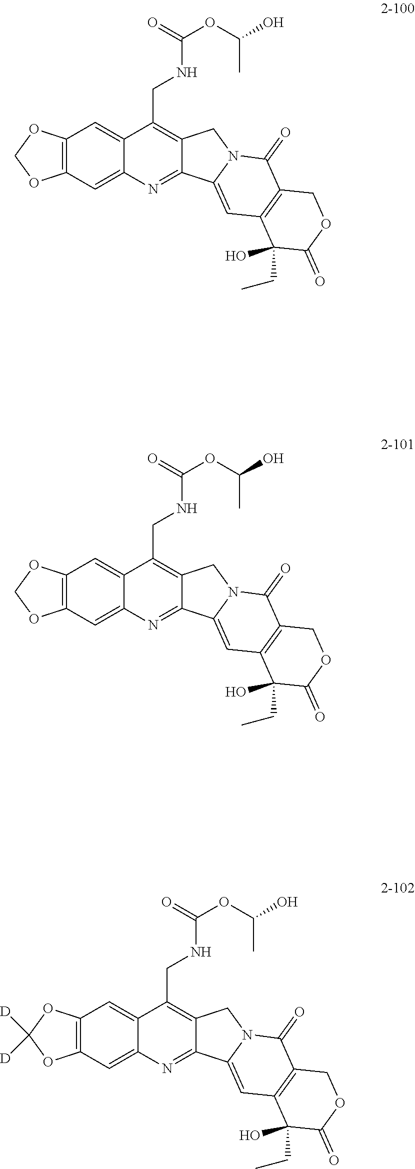 [SMILES: CC[C@@]1(O)C(=O)OCc2c1cc1n(c2=O)Cc2c-1nc1cc3c(cc1c2CNC(=O)O[C@@H](C)O)OCO3 ; CC[C@@]1(O)C(=O)OCc2c1cc1n(c2=O)Cc2c-1nc1cc3c(cc1c2CNC(=O)O[C@H](C)O)OCO3 ; [2H]C1([2H])Oc2cc3nc4c(c(CNC(=O)O[C@@H](C)O)c3cc2O1)Cn1c-4cc2c(c1=O)COC(=O)[C@]2(O)CC]

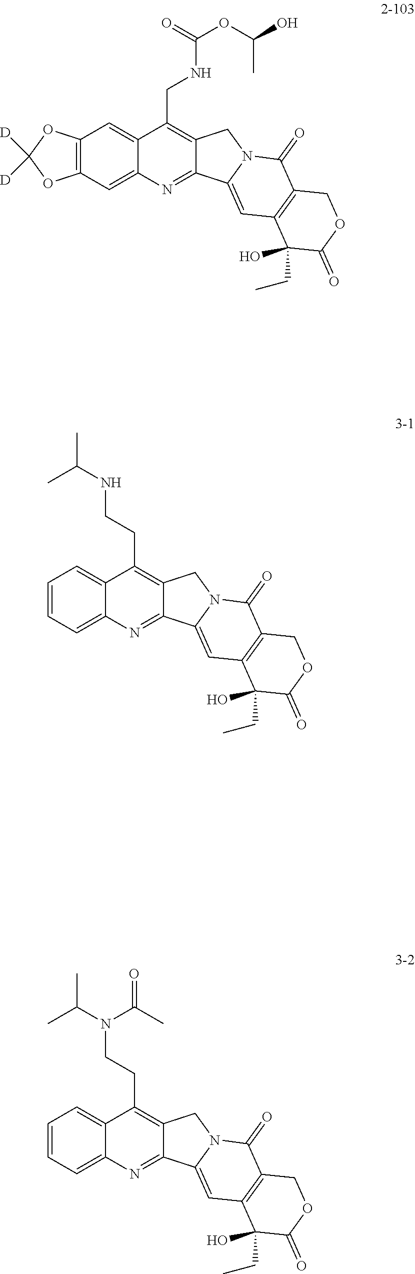 [SMILES: [2H]C1([2H])Oc2cc3nc4c(c(CNC(=O)O[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C]

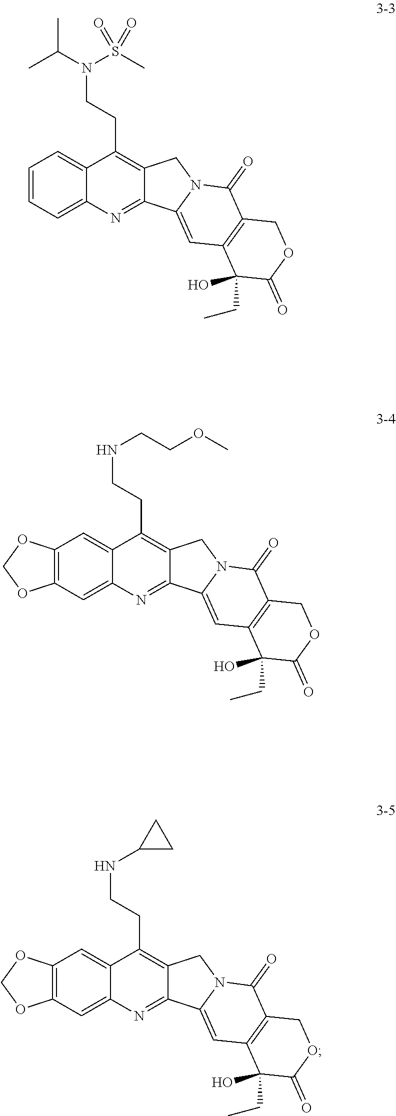 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1cc3c(cc1c2CCNC1CC1)OCO3]

preferably, the cytotoxic drug is selected from the group consisting of the following compounds or isotopically labeled compounds thereof:

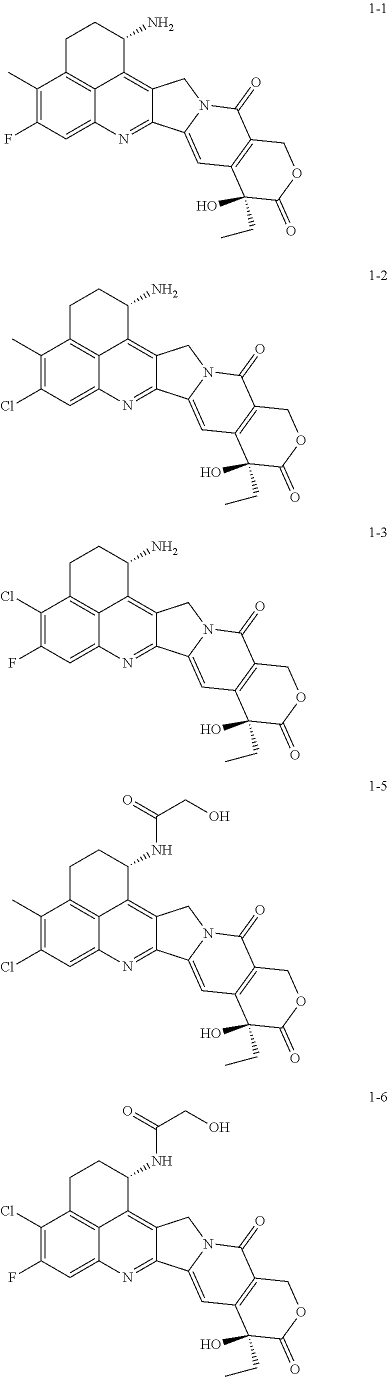 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3]

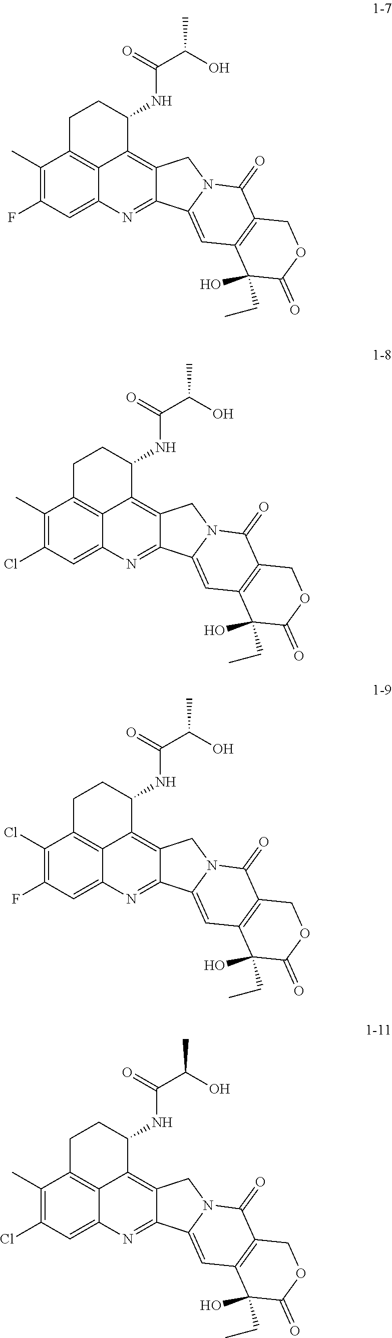 [SMILES: CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3]

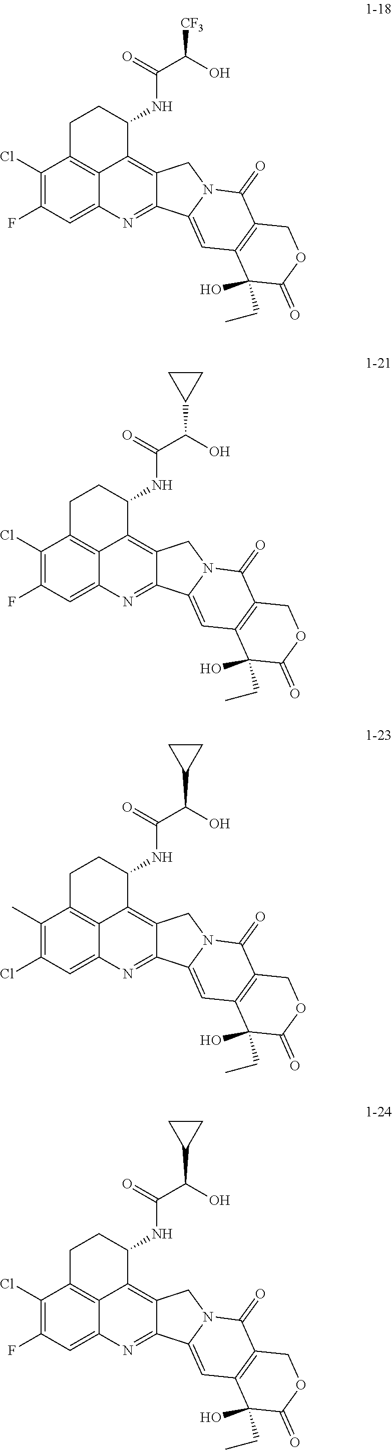 [SMILES: CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3]

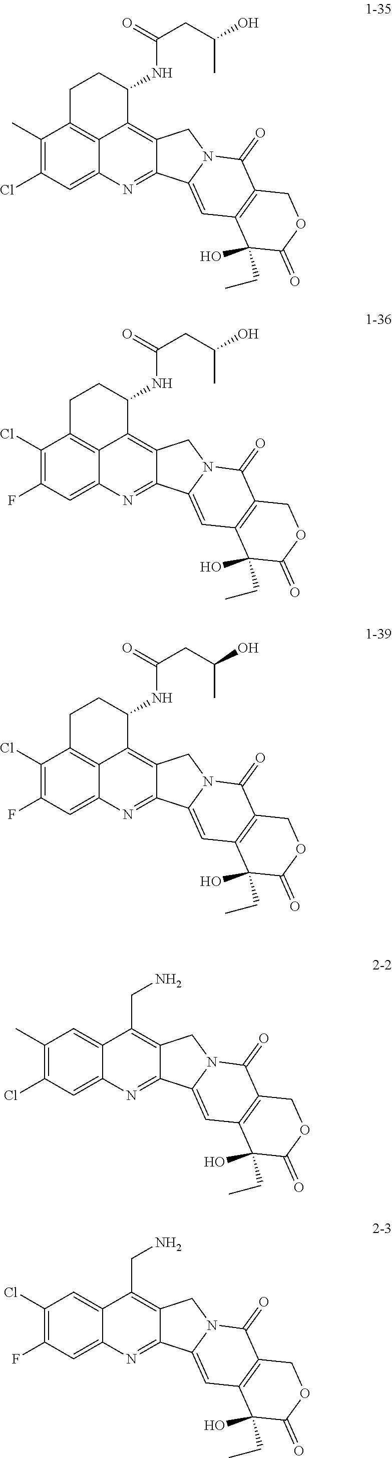 [SMILES: CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN]

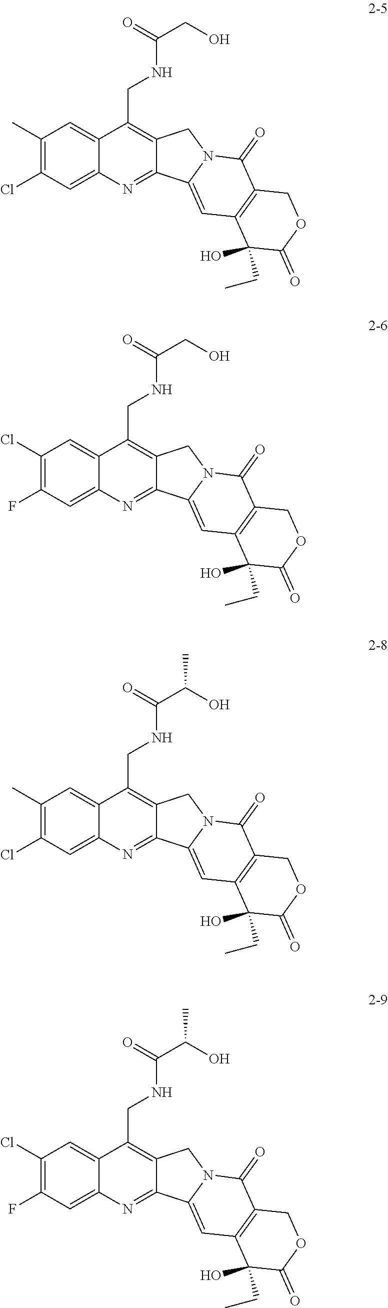 [SMILES: CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O]

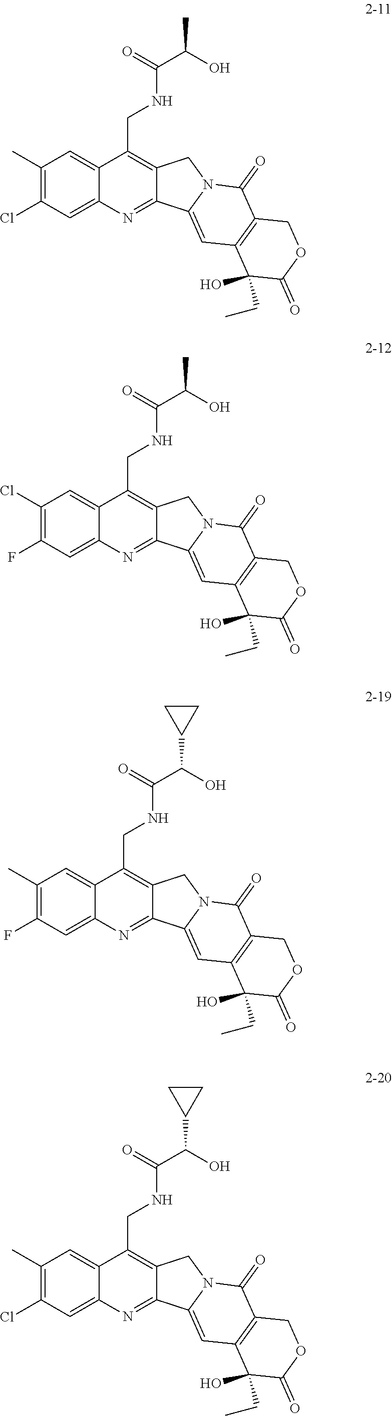 [SMILES: CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](O)C1CC1 ; CC[C@@]1(O)C(=O)OCc2c1cc1n(c2=O)Cc2c-1nc1cc(Cl)c(C)cc1c2CNC(=O)[C@@H](O)C1CC1]

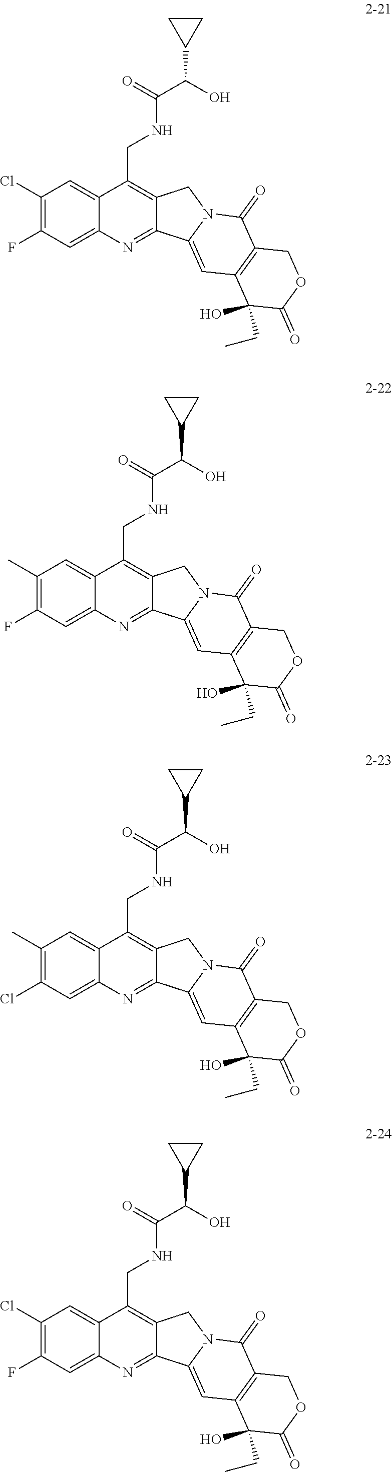 [SMILES: CC[C@@]1(O)C(=O)OCc2c1cc1n(c2=O)Cc2c-1nc1cc(F)c(Cl)cc1c2CNC(=O)[C@@H](O)C1CC1 ; CC[C@@]1(O)C(=O)OCc2c1cc1n(c2=O)Cc2c-1nc1cc(F)c(C)cc1c2CNC(=O)[C@H](O)C1CC1 ; CC[C@@]1(O)C(=O)OCc2c1cc1n(c2=O)Cc2c-1nc1cc(Cl)c(C)cc1c2CNC(=O)[C@H](O)C1CC1 ; CC[C@@]1(O)C(=O)OCc2c1cc1n(c2=O)Cc2c-1nc1cc(F)c(Cl)cc1c2CNC(=O)[C@H](O)C1CC1]

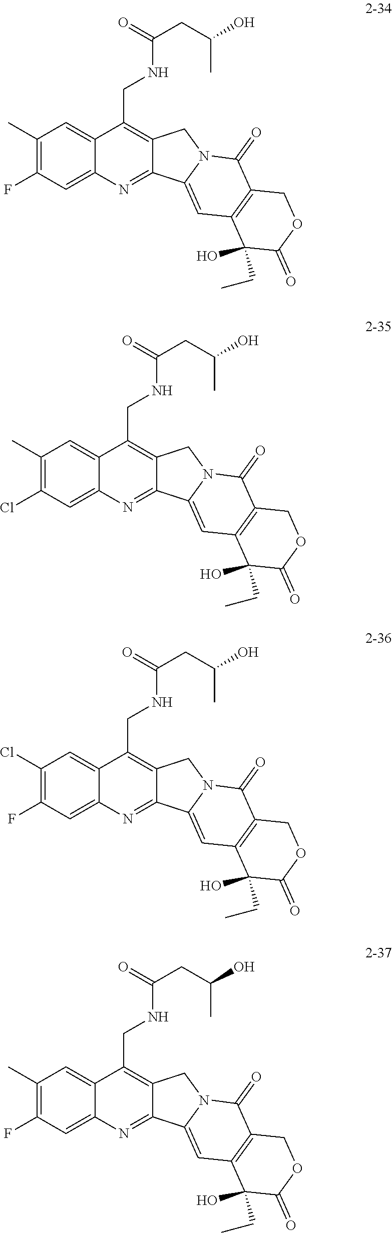 [SMILES: CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O]

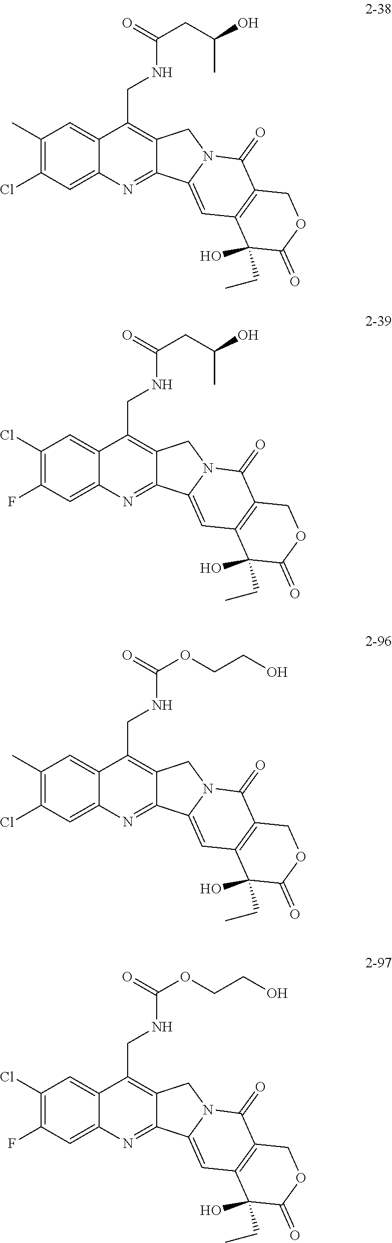 [SMILES: CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2CNC(=O)OCCO ; CC[C@@]1(O)C(=O)OCc2c1cc1n(c2=O)Cc2c-1nc1cc(F)c(Cl)cc1c2CNC(=O)OCCO]

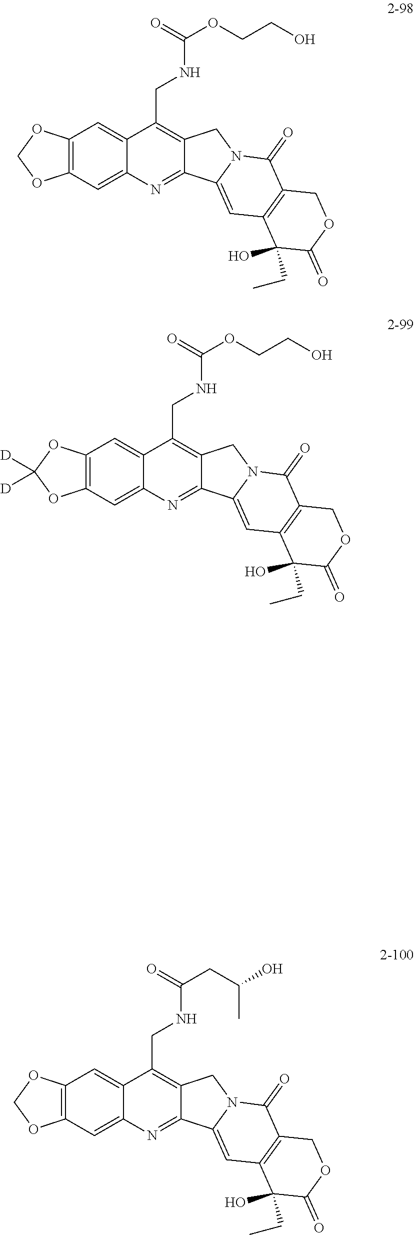 [SMILES: CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC ; CC[C@@]1(O)C(=O)OCc2c1cc1n(c2=O)Cc2c-1nc1cc3c(cc1c2CNC(=O)C[C@@H](C)O)OCO3]

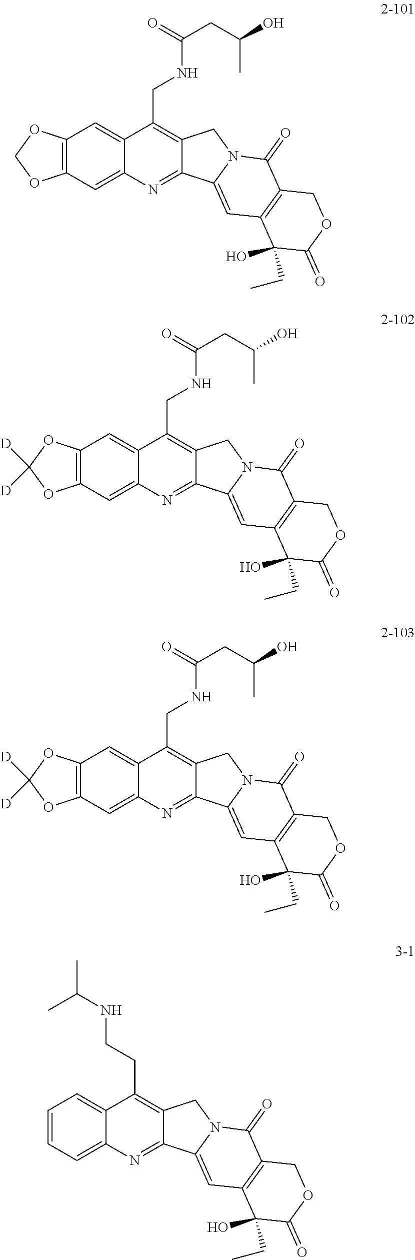 [SMILES: CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C]

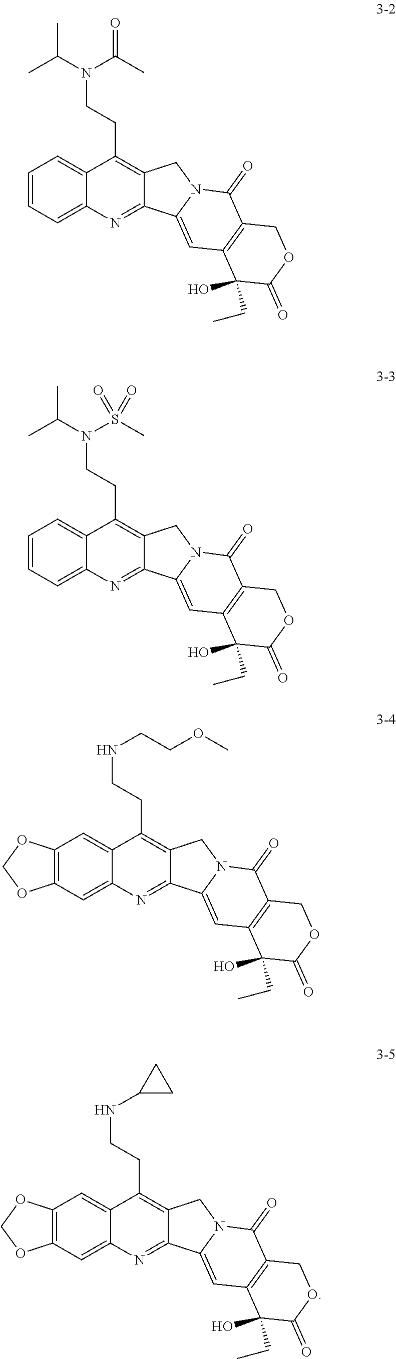 [SMILES: CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1cc3c(cc1c2CCNC1CC1)OCO3]

In some embodiments, the cytotoxic drug is selected from the group consisting of the following compounds or isotopically labeled compounds thereof:

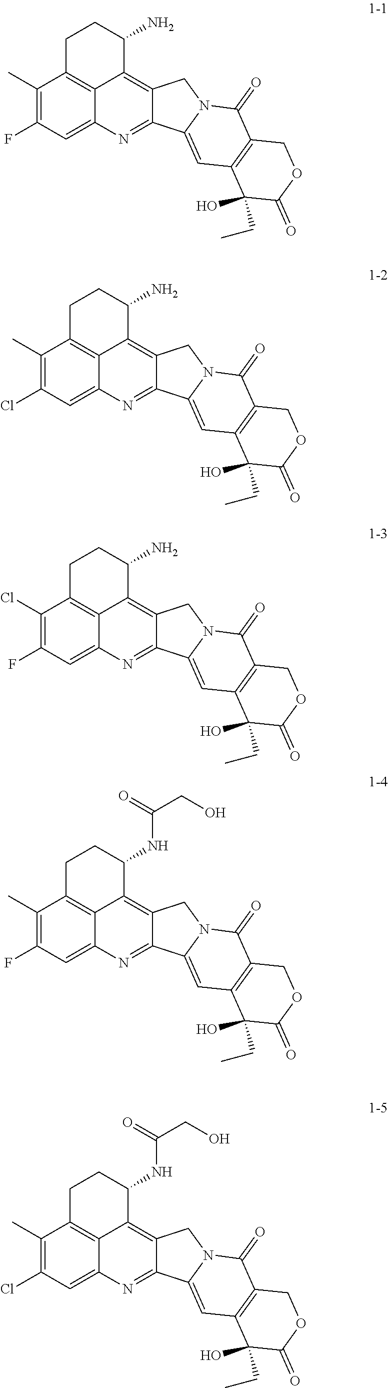 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

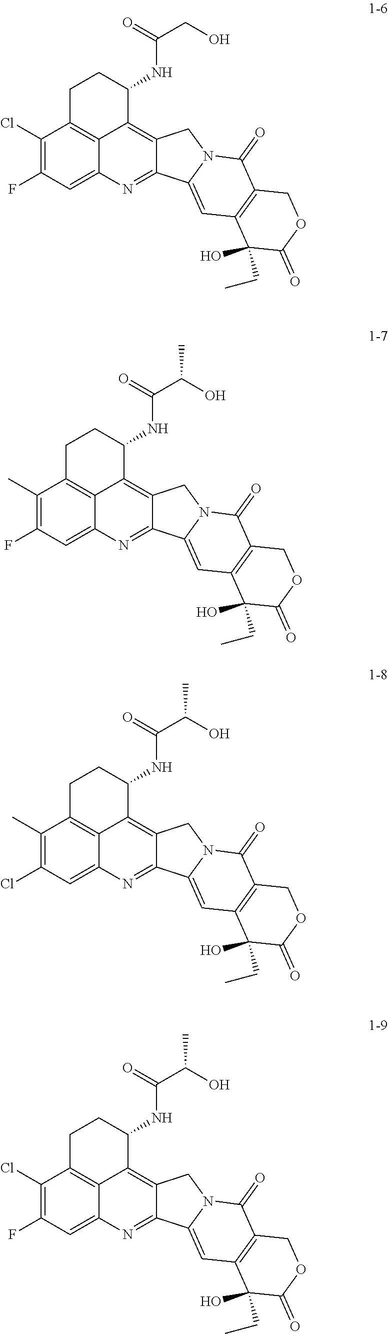 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3]

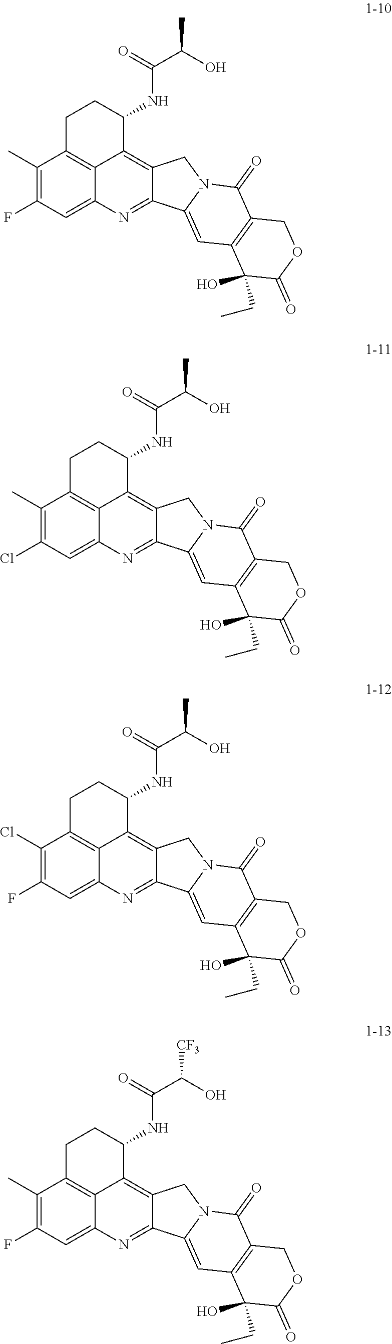 [SMILES: CC[C@@]1(O)C(=O)OCc2c1cc1n(c2=O)Cc2c-1nc1cc(F)c(C)c3c1c2[C@@H](NC(=O)[C@@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@@H](C)O)CC3 ; CC[C@@]1(O)C(=O)OCc2c1cc1n(c2=O)Cc2c-1nc1cc(F)c(C)c3c1c2[C@@H](NC(=O)[C@@H](O)C(F)(F)F)CC3]

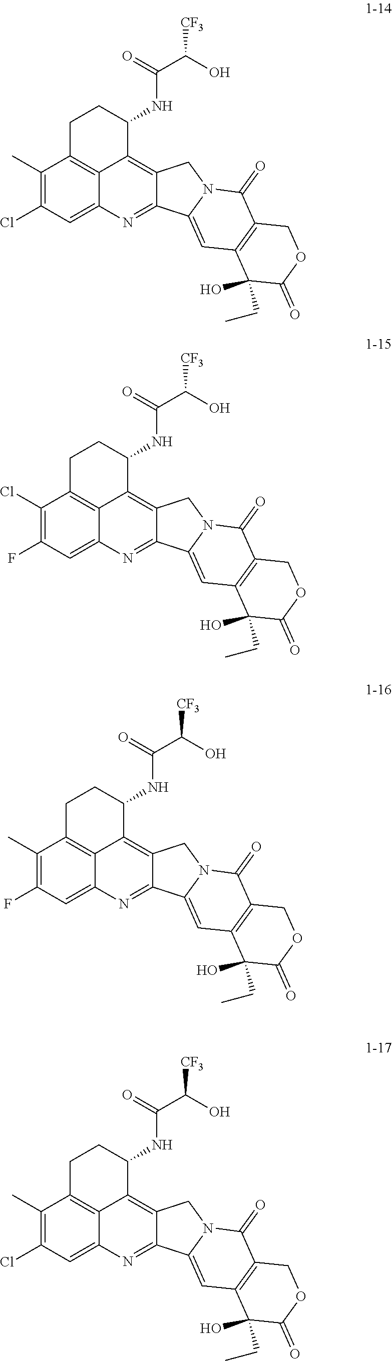 [SMILES: CC[C@@]1(O)C(=O)OCc2c1cc1n(c2=O)Cc2c-1nc1cc(Cl)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(C)c3c1c2[C@@H](NC(=O)[C@H](O)C(F)(F)F)CC3 ; CC[C@@]1(O)C(=O)OCc2c1cc1n(c2=O)Cc2c-1nc1cc(Cl)c(C)c3c1c2[C@@H](NC(=O)[C@H](O)C(F)(F)F)CC3]

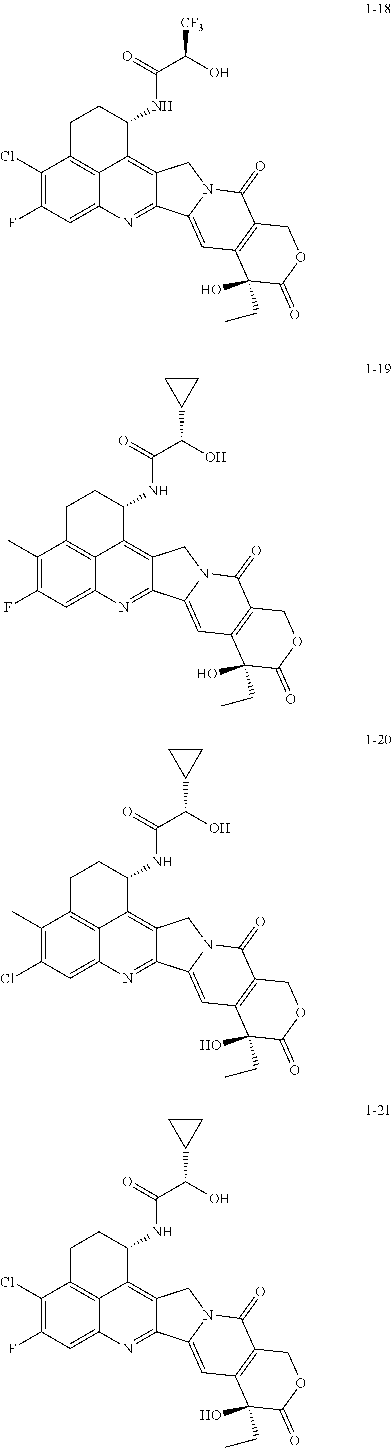 [SMILES: CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(C)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3]

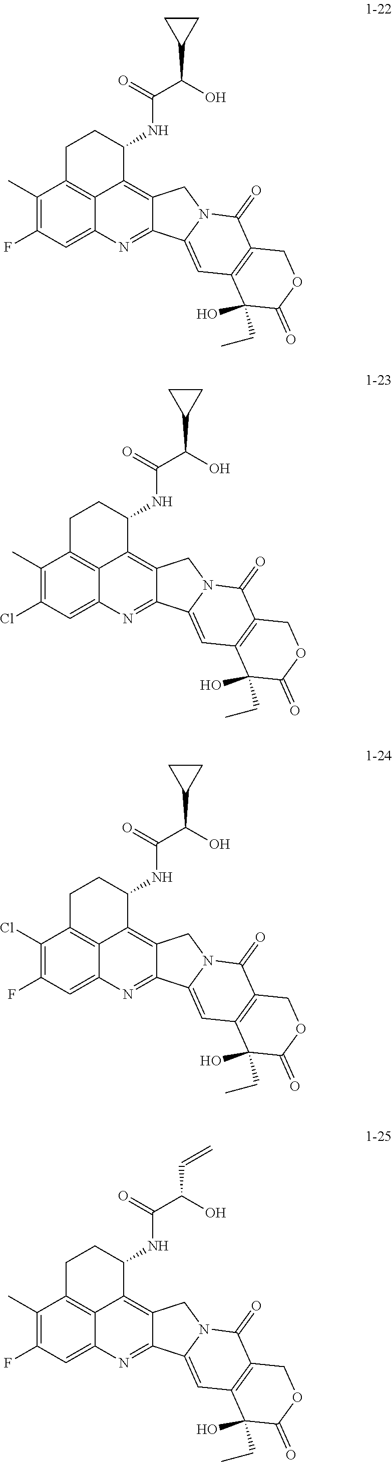 [SMILES: CC[C@@]1(O)C(=O)OCc2c1cc1n(c2=O)Cc2c-1nc1cc(F)c(C)c3c1c2[C@@H](NC(=O)[C@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3 ; C=C[C@H](O)C(=O)N[C@H]1CCc2c(C)c(F)cc3nc4c(c1c23)Cn1c-4cc2c(c1=O)COC(=O)[C@]2(O)CC]

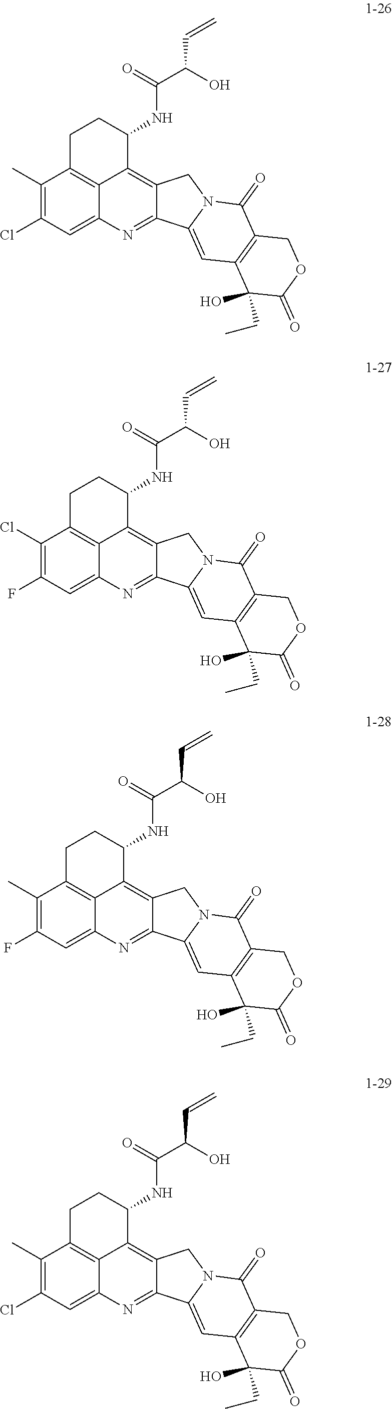

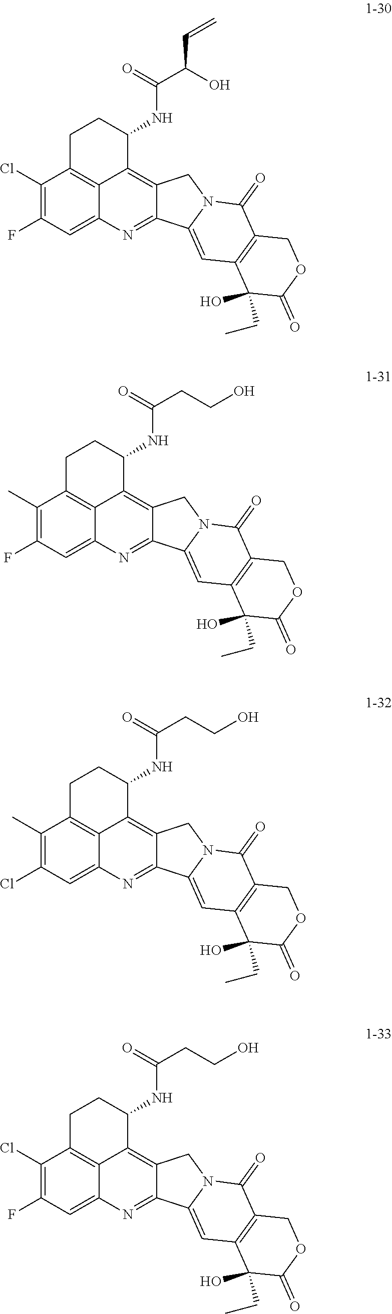 [SMILES: C=C[C@@H](O)C(=O)N[C@H]1CCc2c(Cl)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)CCO)CC3 ; CC[C@@]1(O)C(=O)OCc2c1cc1n(c2=O)Cc2c-1nc1cc(Cl)c(C)c3c1c2[C@@H](NC(=O)CCO)CC3 ; CC[C@@]1(O)C(=O)OCc2c1cc1n(c2=O)Cc2c-1nc1cc(F)c(Cl)c3c1c2[C@@H](NC(=O)CCO)CC3]

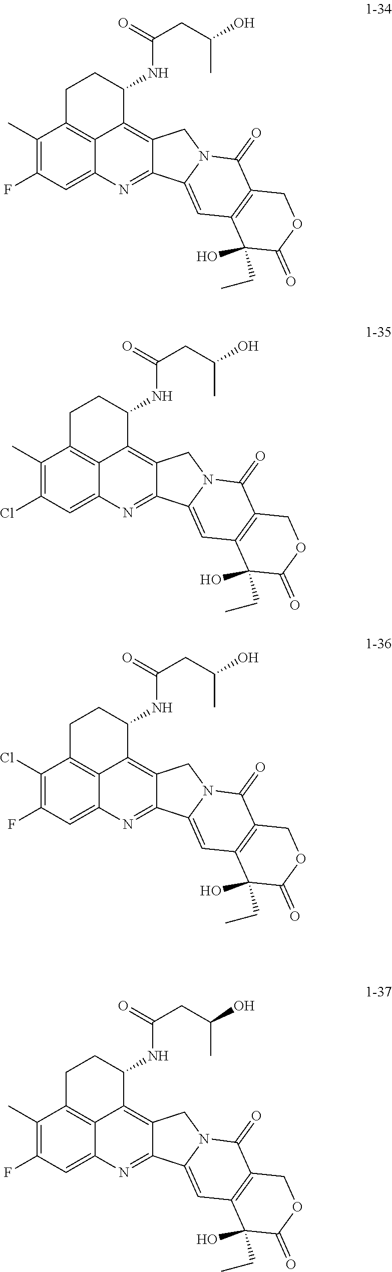 [SMILES: CC[C@@]1(O)C(=O)OCc2c1cc1n(c2=O)Cc2c-1nc1cc(F)c(C)c3c1c2[C@@H](NC(=O)C[C@@H](C)O)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(C)c3c1c2[C@@H](NC(=O)C[C@H](C)O)CC3]

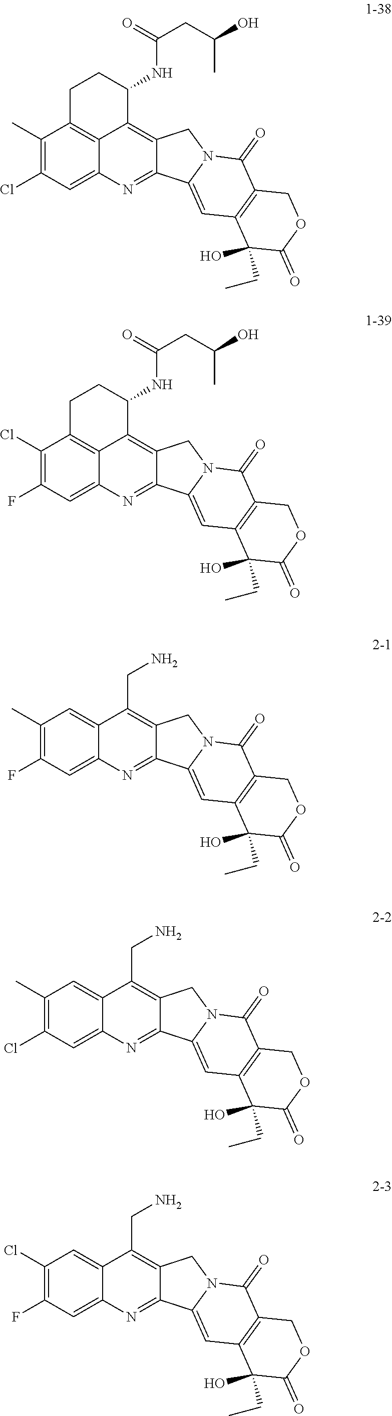 [SMILES: CC[C@@]1(O)C(=O)OCc2c1cc1n(c2=O)Cc2c-1nc1cc(Cl)c(C)c3c1c2[C@@H](NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(F)c(C)cc1c2CN ; CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN]

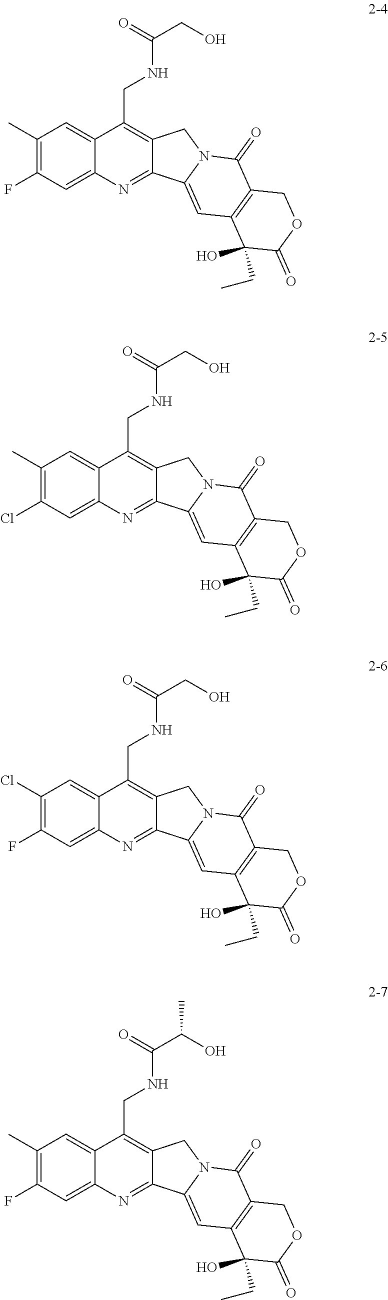 [SMILES: CC[C@@]1(O)C(=O)OCc2c1cc1n(c2=O)Cc2c-1nc1cc(F)c(C)cc1c2CNC(=O)CO ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(F)c(C)cc1c2CNC(=O)[C@H](C)O]

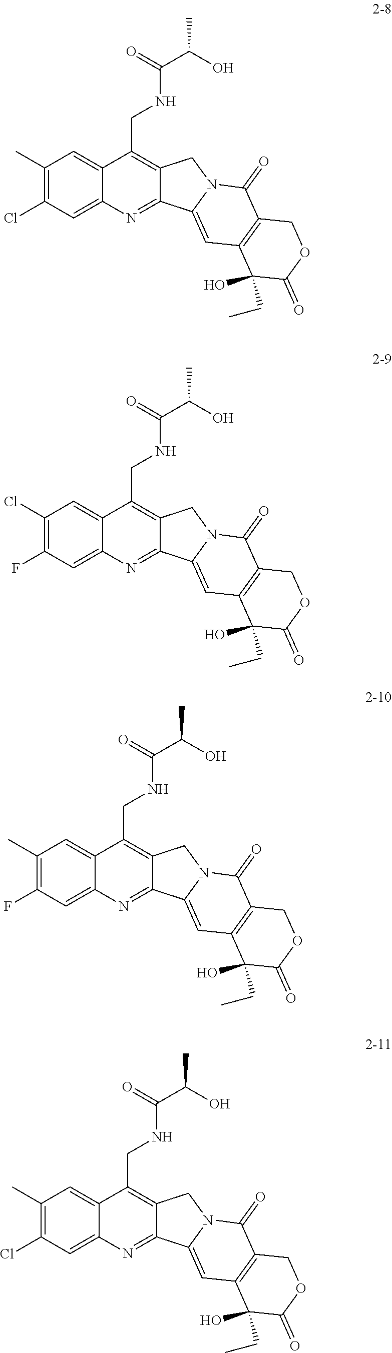 [SMILES: CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(F)c(C)cc1c2CNC(=O)[C@@H](C)O ; CC[C@@]1(O)C(=O)OCc2c1cc1n(c2=O)Cc2c-1nc1cc(Cl)c(C)cc1c2CNC(=O)[C@@H](C)O]

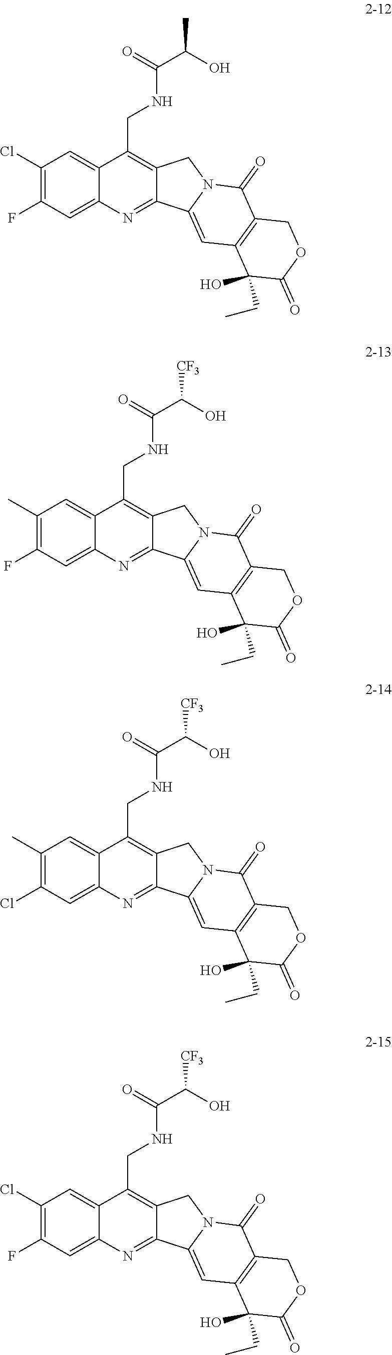 [SMILES: CC[C@@]1(O)C(=O)OCc2c1cc1n(c2=O)Cc2c-1nc1cc(F)c(Cl)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](O)C(F)(F)F ; CC[C@@]1(O)C(=O)OCc2c1cc1n(c2=O)Cc2c-1nc1cc(Cl)c(C)cc1c2CNC(=O)[C@@H](O)C(F)(F)F ; CC[C@@]1(O)C(=O)OCc2c1cc1n(c2=O)Cc2c-1nc1cc(F)c(Cl)cc1c2CNC(=O)[C@@H](O)C(F)(F)F]

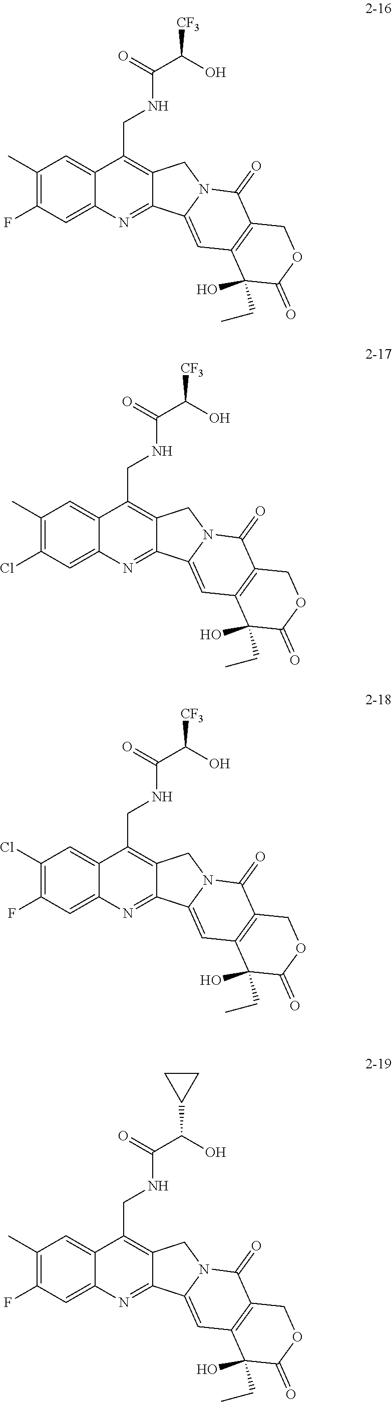 [SMILES: CC[C@@]1(O)C(=O)OCc2c1cc1n(c2=O)Cc2c-1nc1cc(F)c(C)cc1c2CNC(=O)[C@H](O)C(F)(F)F ; CC[C@@]1(O)C(=O)OCc2c1cc1n(c2=O)Cc2c-1nc1cc(Cl)c(C)cc1c2CNC(=O)[C@H](O)C(F)(F)F ; CC[C@@]1(O)C(=O)OCc2c1cc1n(c2=O)Cc2c-1nc1cc(F)c(Cl)cc1c2CNC(=O)[C@H](O)C(F)(F)F ; CC[C@@]1(O)C(=O)OCc2c1cc1n(c2=O)Cc2c-1nc1cc(F)c(C)cc1c2CNC(=O)[C@@H](O)C1CC1]

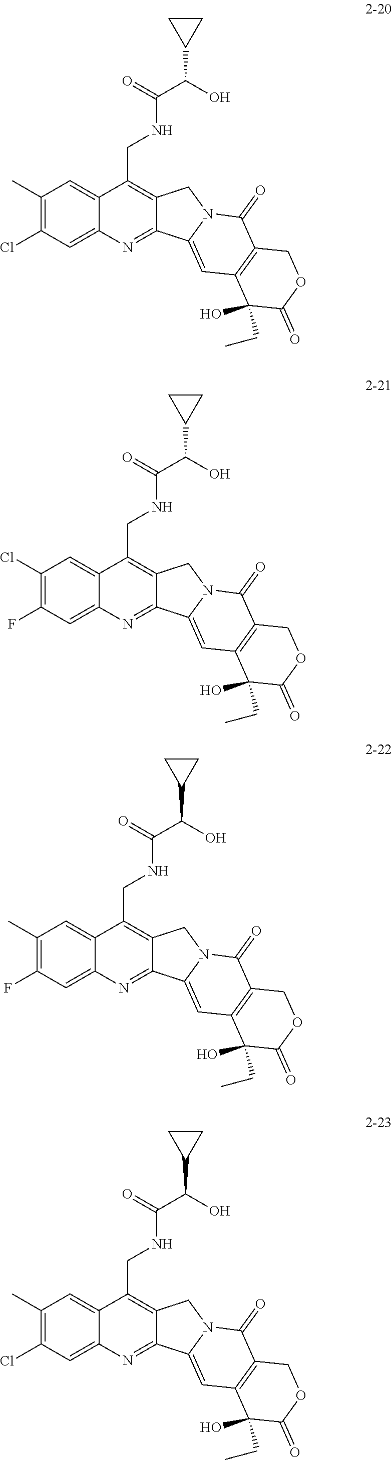

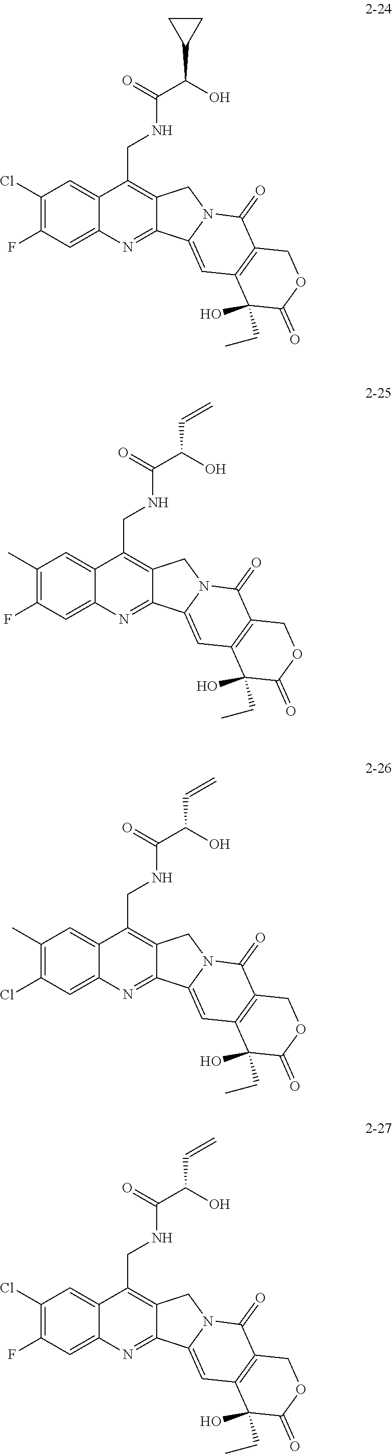

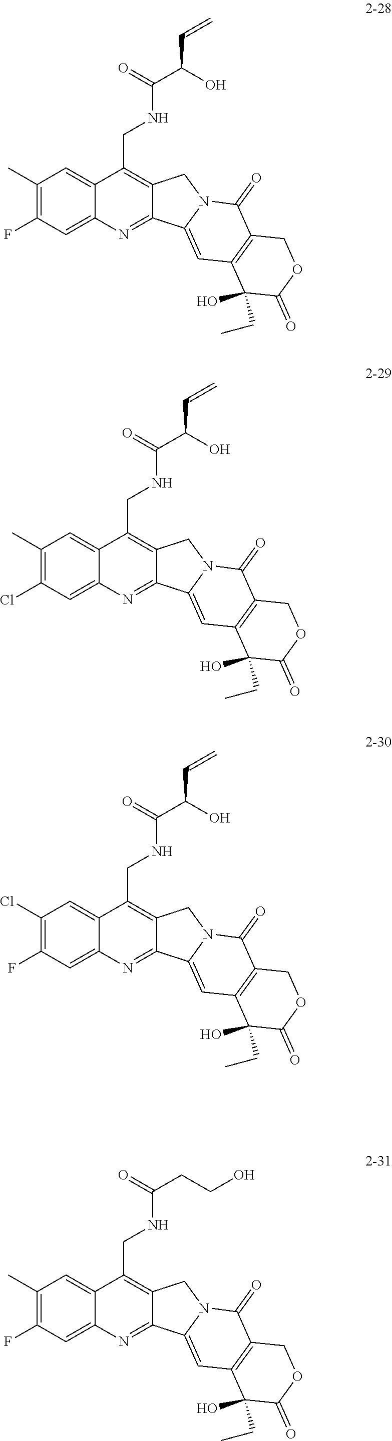 [SMILES: C=C[C@@H](O)C(=O)NCc1c2c(nc3cc(F)c(C)cc13)-c1cc3c(c(=O)n1C2)COC(=O)[C@]3(O)CC ; C=C[C@@H](O)C(=O)NCc1c2c(nc3cc(Cl)c(C)cc13)-c1cc3c(c(=O)n1C2)COC(=O)[C@]3(O)CC ; C=C[C@@H](O)C(=O)NCc1c2c(nc3cc(F)c(Cl)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2CNC(=O)CCO]

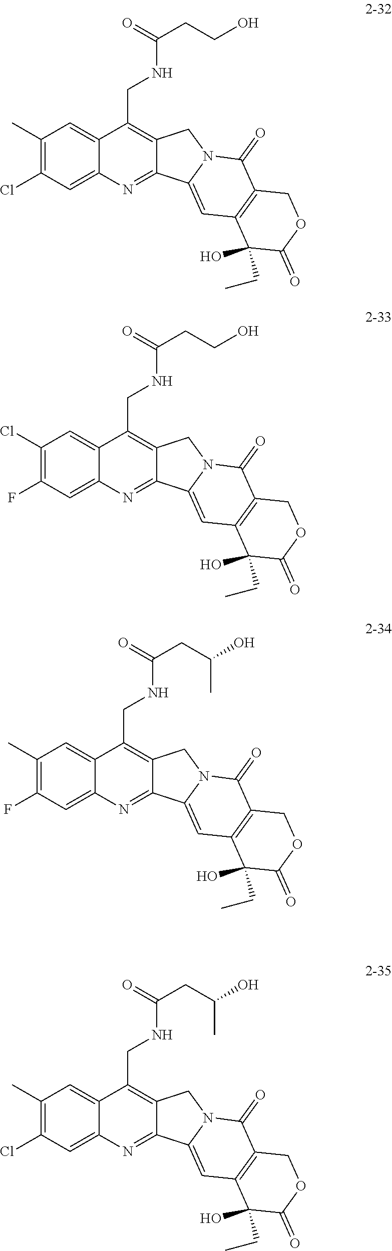

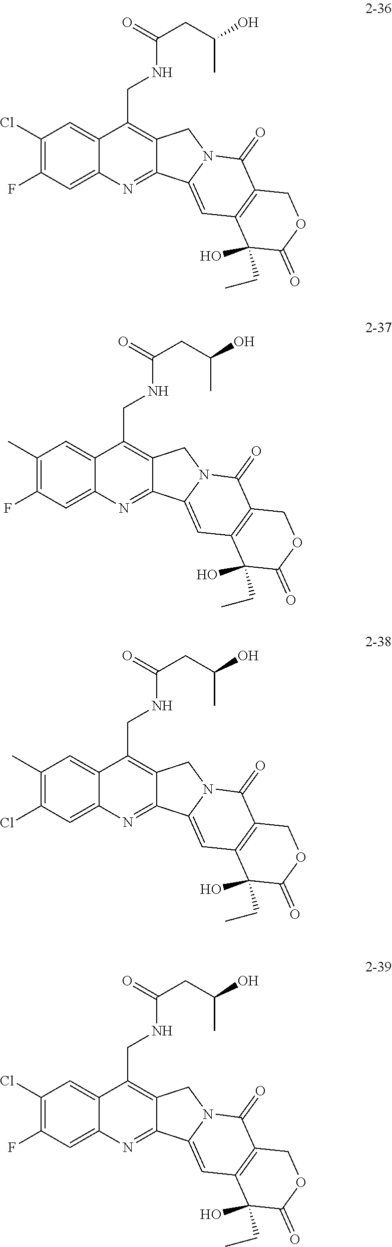 [SMILES: CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O]

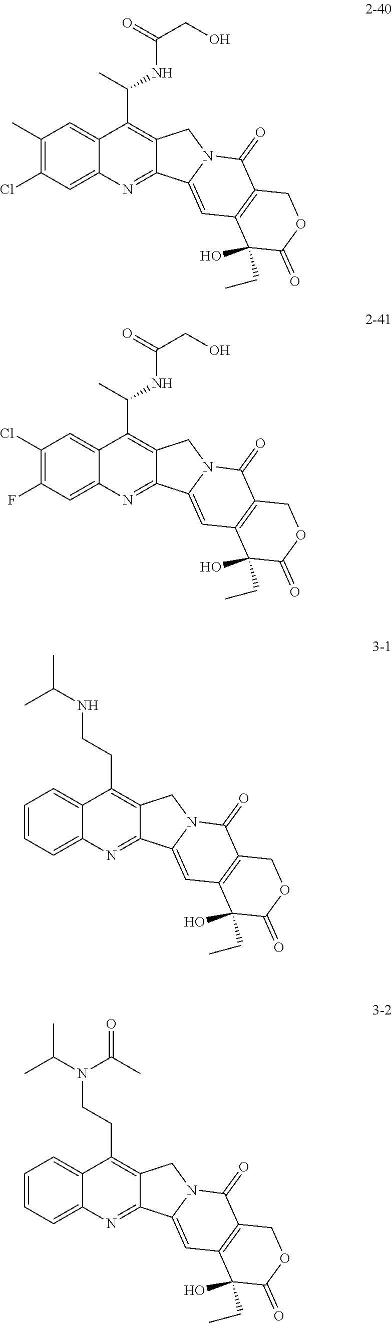 [SMILES: CC[C@@]1(O)C(=O)OCc2c1cc1n(c2=O)Cc2c-1nc1cc(Cl)c(C)cc1c2[C@H](C)NC(=O)CO ; CC[C@@]1(O)C(=O)OCc2c1cc1n(c2=O)Cc2c-1nc1cc(F)c(Cl)cc1c2[C@H](C)NC(=O)CO ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C]

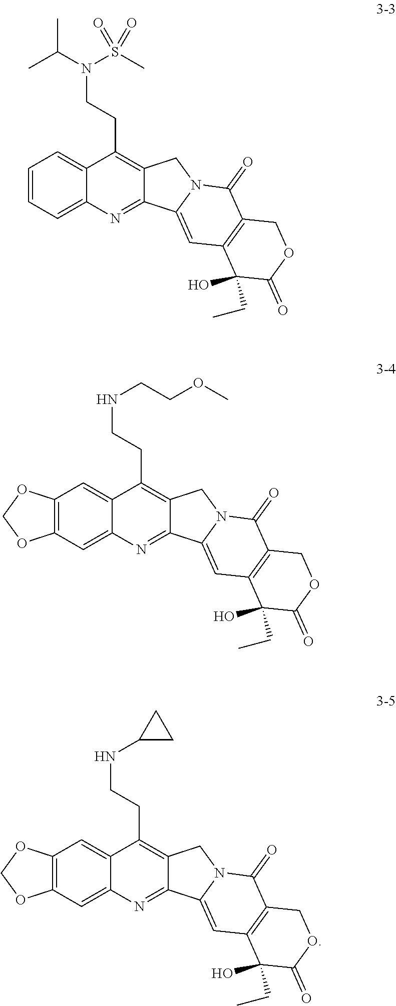 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1cc3c(cc1c2CCNC1CC1)OCO3]

In some embodiments, the cytotoxic drug is selected from the group consisting of the following Formula III and Formula IV

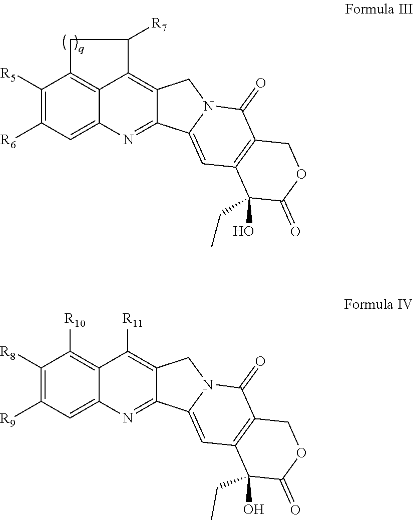

wherein, R₅, R₆ are each independently selected from the group consisting of H, OH, —NH₂, —NH(C₁₋₆ alkyl), C₁₋₆ alkyl and halogen; the C₁₋₆ alkyl is optionally further substituted with one or more substituents selected from the group consisting of halogen, hydroxyl, C₁₋₆ haloalkyl, C₃₋₆ cycloalkyl; or, R₅ and R₆ together with adjacent carbon atoms (ring-forming carbon atoms connecting R₅ and R₆, respectively) form a 5-membered oxygen-containing heterocycle

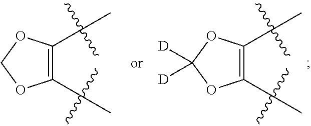 [SMILES: *C1=C(*)OCO1 ; *C1=C(*)OC([2H])([2H])O1]

R₇ is selected from the group consisting of H, —OH, —NH₂, —NH(C₁₋₆ alkyl), and —NH—CO—(C₁₋₆ alkylene)-OH; the C₁₋₆ alkyl and C₁₋₆ alkylene are optionally further substituted with one or more substituents selected from the group consisting of halogen, hydroxyl, C₁₋₆ haloalkyl, C₃₋₆ cycloalkyl;
q is 1 or 2;
R₈ and R₉ are independently selected from the group consisting of H, halogen and hydroxyl; or R₄ and R₅ are connected to the connected carbon atoms to form a 5- to 6-membered oxygen-containing heterocycle; the 5- to 6-membered oxygen-containing heterocycle is optionally substituted with one or more substituents selected from the group consisting of deuterium (D), halogen, hydroxyl, C₁₋₆ haloalkyl, C₃₋₆ cycloalkyl.
R₁₀ is selected from the group consisting of hydrogen or —C₁₋₄ alkylene-NRₐR_{b};
R₁₁ is selected from the group consisting of C₁₋₆ alkyl and —C₁₋₄ alkylene-NRₐR_{b}; and
wherein Rₐ, R_{b} are each independently selected at each occurrence from the group consisting of H, C₁₋₆ alkyl, C₃₋₆ cycloalkyl, —SO₂—C₁₋₆ alkyl, —(C═O)—OC₁₋₆ alkyl and —CO—C₁₋₆ alkyl; the C₁₋₆ alkyl and C₁₋₄ alkylene are optionally further substituted with one or more substituents selected from the group consisting of halogen, hydroxyl, C₁₋₆ haloalkyl, C₁₋₆ alkoxy, C₃₋₆ cycloalkyl.

In some embodiments, R₅, R₆ are each independently selected from the group consisting of OH, —NH₂, C₁₋₄ alkyl and halogen; the C₁₋₄ alkyl is optionally further substituted with one or more substituents selected from the group consisting of halogen, hydroxyl, C₁₋₄ haloalkyl, C₃₋₆ cycloalkyl. In some embodiments, R₅ and R₆ together with adjacent carbon atoms (ring-forming carbon atoms connecting R₅ and R₆, respectively) form a 5-membered oxygen-containing heterocycle

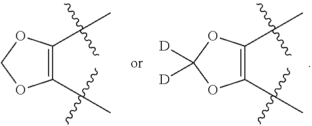 [SMILES: *C1=C(*)OCO1 ; *C1=C(*)OC([2H])([2H])O1]

In some embodiments, R₇ is selected from the group consisting of —H, —NH₂, —NH(C₁₋₄ alkyl), and —NH—CO—(C₁₋₄ alkylene)-OH; the C₁₋₄ alkyl and C₁₋₄ alkylene are optionally further substituted with one or more substituents selected from the group consisting of halogen, hydroxyl, C₁₋₄ haloalkyl, C₃₋₆ cycloalkyl.

In some embodiments, Rₐ, R_{b} are independently selected at each occurrence from the group consisting of H, C₁₋₄ alkyl, C₃₋₆ cycloalkyl, —SO₂—C₁₋₄ alkyl and —CO—C₁₋₄ alkyl; the C₁₋₄ alkyl is optionally further substituted with one or more substituents selected from the group consisting of halogen, hydroxyl, C₁₋₆ haloalkyl, C₃₋₆ cycloalkyl.

In some embodiments, R₅, R₆ are each independently selected from the group consisting of H, OH, —NH₂, C₁₋₆ alkyl and halogen;
R₇ is selected from the group consisting of —H, —NH₂, —NH(C₁₋₆ alkyl), and —NH—CO—(C₁₋₆ alkylene)-OH; the C₁₋₆ alkylene is optionally substituted with C₃₋₆ cycloalkyl; and
q is 2.

In some embodiments, R₅, R₆ are each independently selected from the group consisting of OH, —NH₂, C₁₋₄ alkyl and halogen. In some embodiments, R₅, R₆ are each independently selected from the group consisting of OH, —NH₂, methyl, ethyl, n-propyl, isopropyl and halogen. In some embodiments, R₅, R₆ are each independently selected from the group consisting of OH, —NH₂, methyl, fluorine and chlorine.

In some embodiments, R₇ is selected from the group consisting of —H, —NH₂, —NH(C₁₋₄ alkyl), and —NH—CO—(C₁₋₄ alkylene)-OH. In some embodiments, R₇ is selected from the group consisting of —H, —NH₂, —NHCH₃, —NHCH₂CH₃, —NHCH(CH₃)₂, —NH—CO—CH₂—OH, —NH—CO—CH₂CH₂—OH, —NH—CO—CH₂(CH₃)—OH, and —NH—CO—CH₂CH(CH₃)—OH. In some embodiments, R₇ is selected from the group consisting of —H, —NH₂, —NH—CO—CH₂—OH, and —NH—CO—CH₂CH(CH₃)—OH.

In some embodiments, R₈ and R₉ are each independently selected from the group consisting of H, halogen or hydroxyl, R₁₀ is selected from the group consisting of hydrogen; and
R₁₁ is selected from the group consisting of C₁₋₆ alkyl and —C₁₋₄ alkylene-NRₐR_{b};
wherein Rₐ, R_{b} are each independently selected at each occurrence from the group consisting of H, C₁₋₆ alkyl, C₃₋₆ cycloalkyl, —SO₂—C₁₋₆ alkyl, —(C═O)—OC₁₋₆ alkyl and —CO—C₁₋₆ alkyl; the C₁₋₆ alkyl and C₁₋₄ alkylene are optionally further substituted with one or more substituents selected from the group consisting of halogen, hydroxyl, C₁₋₆ haloalkyl, C₁₋₆ alkoxy, C₃₋₆ cycloalkyl.

In some embodiments, R₁₁ is selected from the group consisting of C₁₋₄ alkyl and —C₁₋₄ alkylene-NRₐR_{b};
wherein Rₐ, R_{b} are independently selected at each occurrence from the group consisting of H, C₁₋₄ alkyl, C₃₋₆ cycloalkyl, —SO₂—C₁₋₄ alkyl, —(C═O)—OC₁₋₄ alkyl and —CO—C₁₋₄ alkyl; the C₁₋₄ alkyl and C₁₋₄ alkylene are optionally further substituted with one or more substituents selected from the group consisting of halogen, hydroxyl, C₁₋₄ haloalkyl, C₁₋₄ alkoxy, C₃₋₆ cycloalkyl.

In some embodiments, R₁₁ is selected from the group consisting of ethyl, —CH₂NH—CO—CH(OH)-cyclopropyl, —CH₂NH—CO—CH₂CH(OH)—CH₃ and —CH₂CH₂—N(CH(CH₃)₂)(SO₂—CH₃).

In some embodiments, the cytotoxic drug is selected from the group consisting of the following compounds or isotopically labeled compounds thereof:

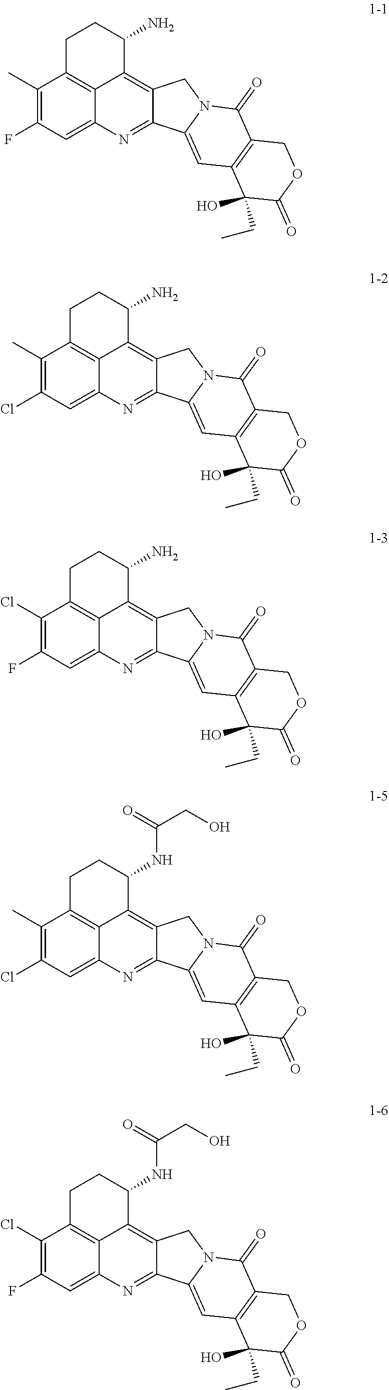 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3]

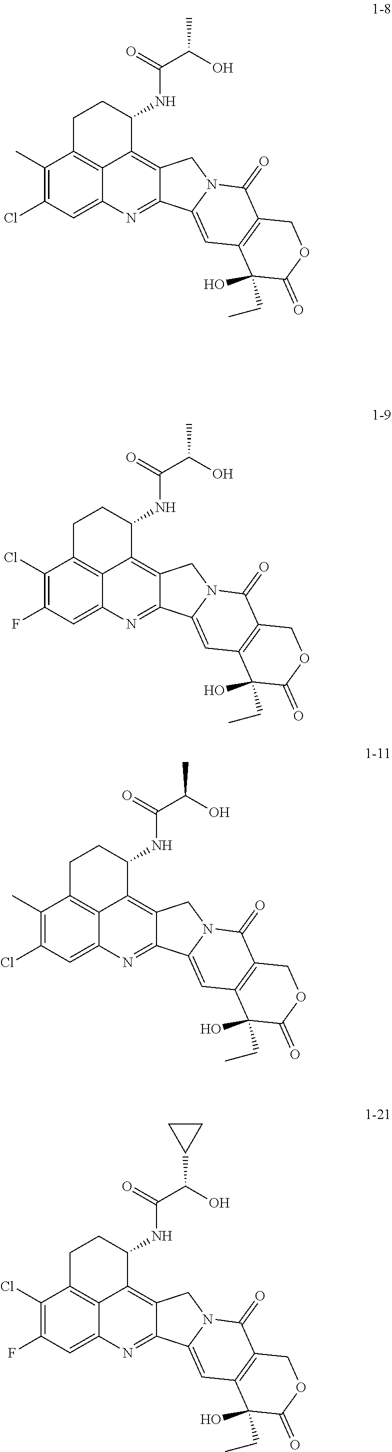 [SMILES: CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3]

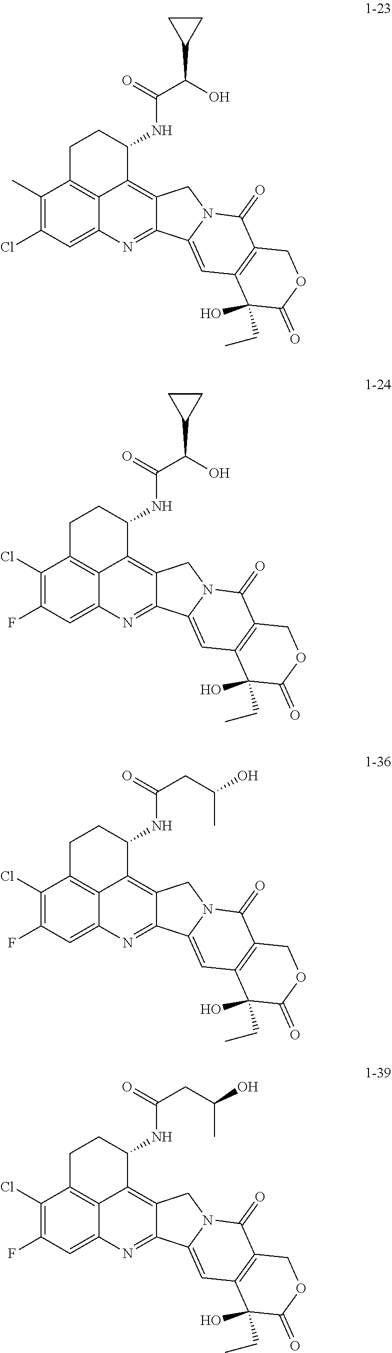 [SMILES: CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3]

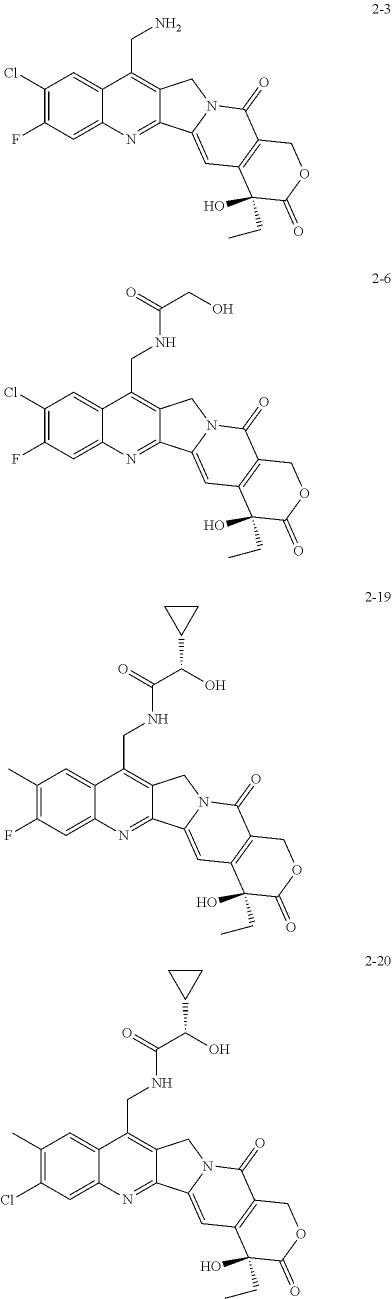

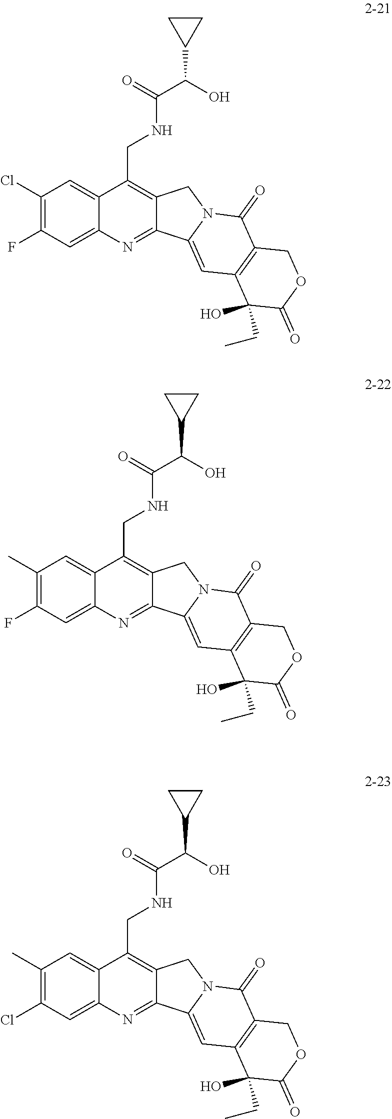 [SMILES: CC[C@@]1(O)C(=O)OCc2c1cc1n(c2=O)Cc2c-1nc1cc(F)c(Cl)cc1c2CNC(=O)[C@@H](O)C1CC1 ; CC[C@@]1(O)C(=O)OCc2c1cc1n(c2=O)Cc2c-1nc1cc(F)c(C)cc1c2CNC(=O)[C@H](O)C1CC1 ; CC[C@@]1(O)C(=O)OCc2c1cc1n(c2=O)Cc2c-1nc1cc(Cl)c(C)cc1c2CNC(=O)[C@H](O)C1CC1]

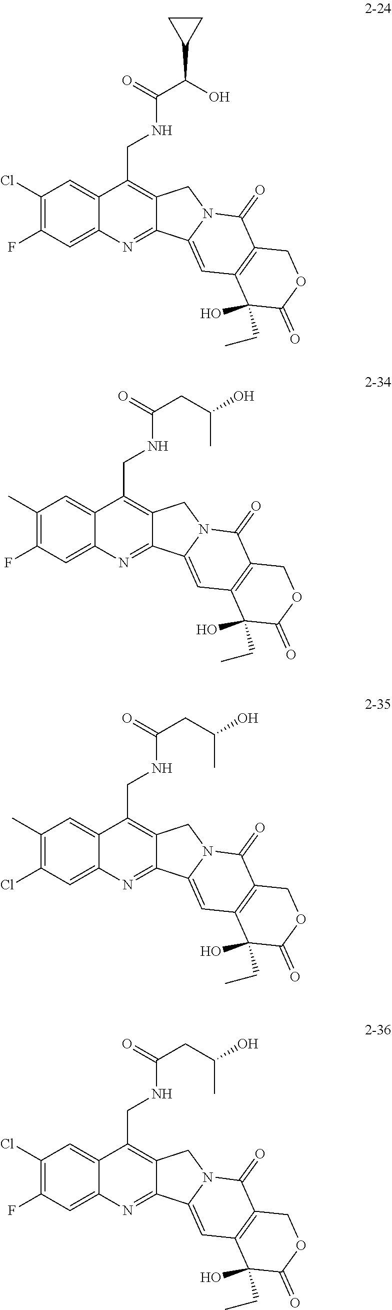 [SMILES: CC[C@@]1(O)C(=O)OCc2c1cc1n(c2=O)Cc2c-1nc1cc(F)c(Cl)cc1c2CNC(=O)[C@H](O)C1CC1 ; CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O]

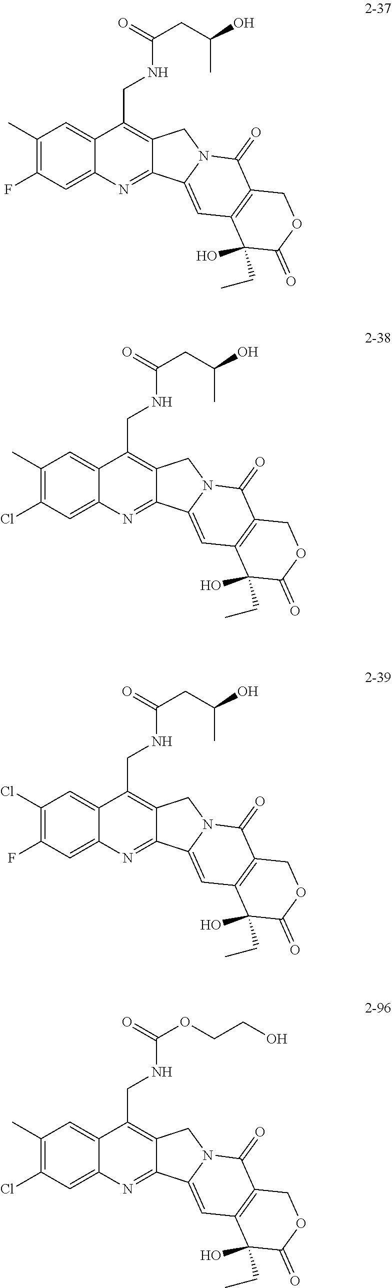 [SMILES: CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2CNC(=O)OCCO]

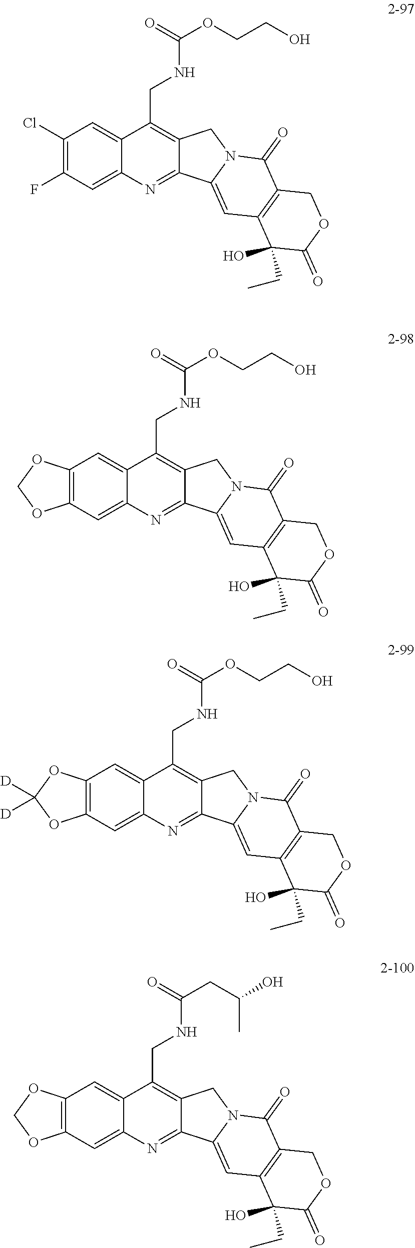 [SMILES: CC[C@@]1(O)C(=O)OCc2c1cc1n(c2=O)Cc2c-1nc1cc(F)c(Cl)cc1c2CNC(=O)OCCO ; CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC ; CC[C@@]1(O)C(=O)OCc2c1cc1n(c2=O)Cc2c-1nc1cc3c(cc1c2CNC(=O)C[C@@H](C)O)OCO3]

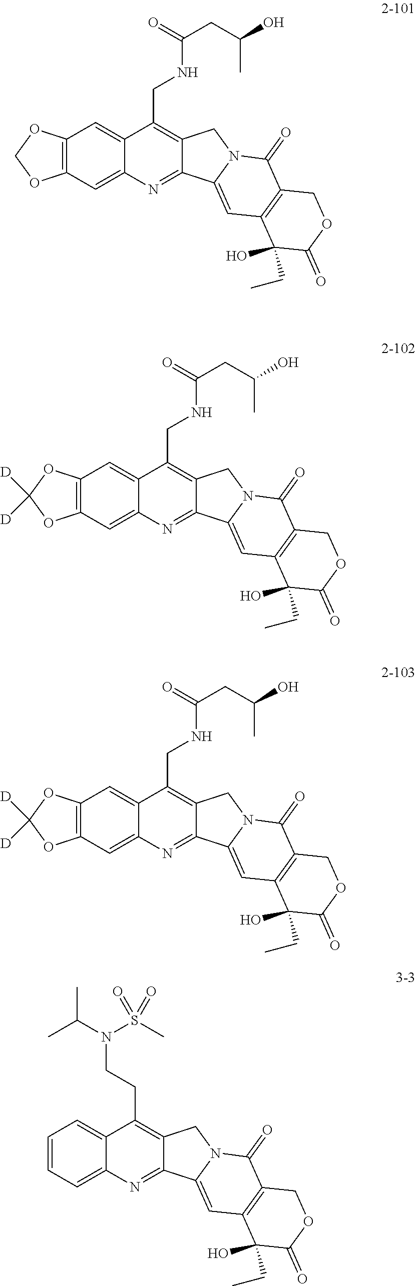 [SMILES: CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCN(C(C)C)S(C)(=O)=O]

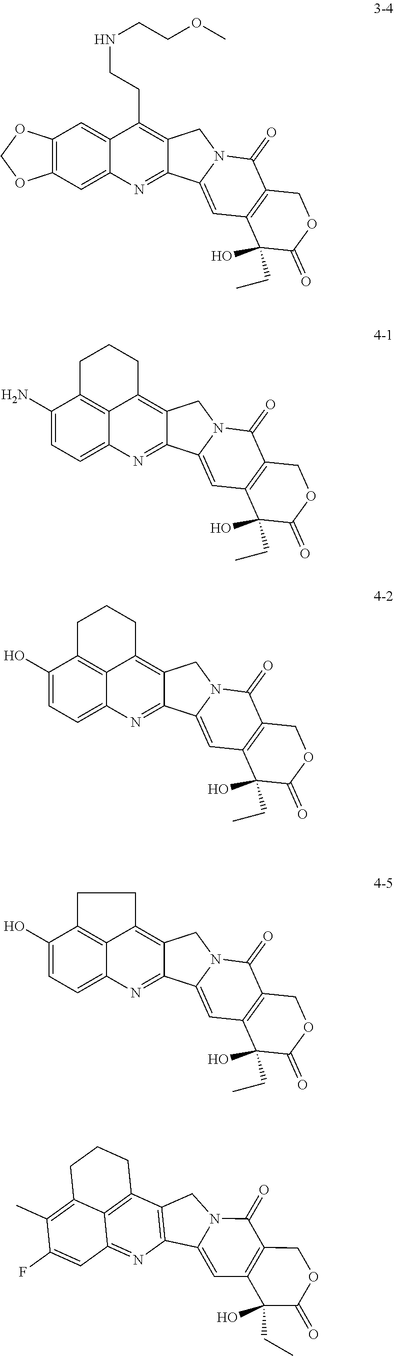 [SMILES: CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2CC3 ; CC[C@@]1(O)C(=O)OCc2c1cc1n(c2=O)Cc2c-1nc1cc(F)c(C)c3c1c2CCC3]

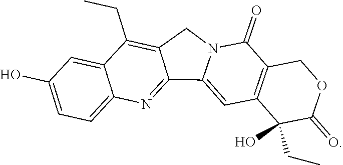 [SMILES: CCc1c2c(nc3ccc(O)cc13)-c1cc3c(c(=O)n1C2)COC(=O)[C@]3(O)CC]

In some embodiments, the cytotoxic drug is selected from the group consisting of the following compounds or isotopically labeled compounds thereof:

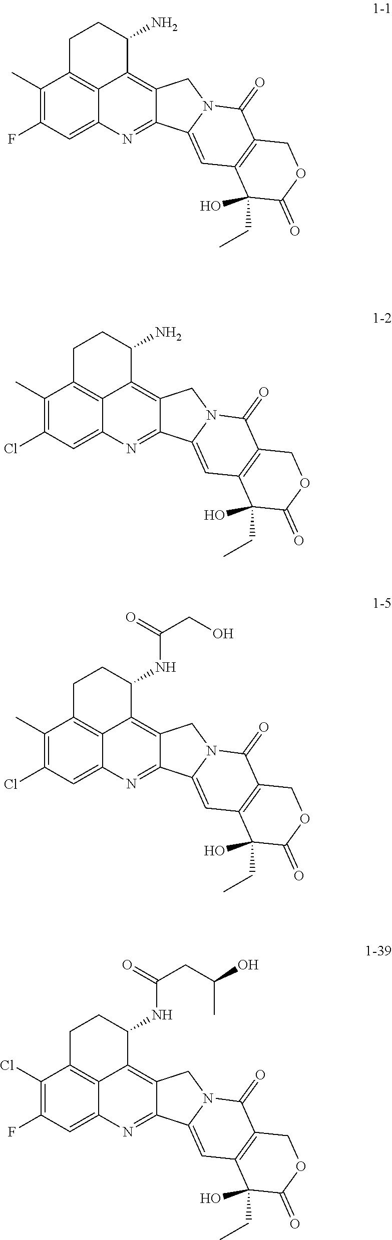 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3]

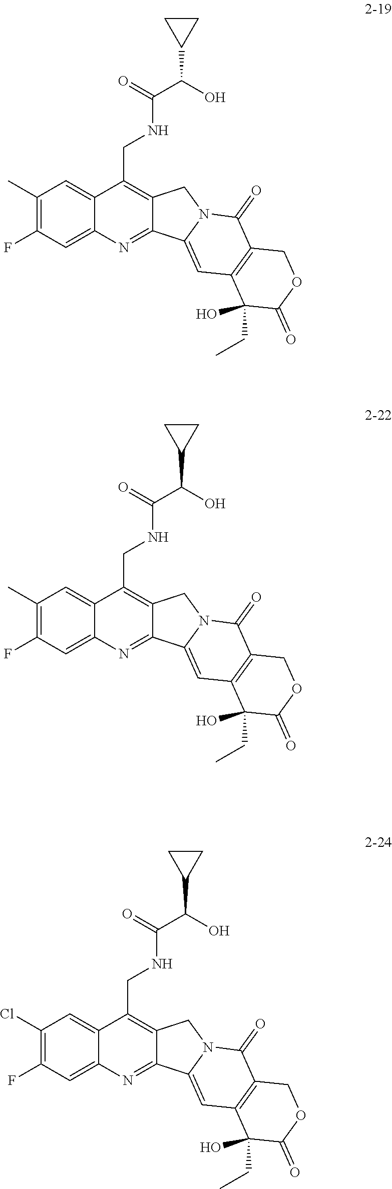 [SMILES: CC[C@@]1(O)C(=O)OCc2c1cc1n(c2=O)Cc2c-1nc1cc(F)c(C)cc1c2CNC(=O)[C@@H](O)C1CC1 ; CC[C@@]1(O)C(=O)OCc2c1cc1n(c2=O)Cc2c-1nc1cc(F)c(C)cc1c2CNC(=O)[C@H](O)C1CC1 ; CC[C@@]1(O)C(=O)OCc2c1cc1n(c2=O)Cc2c-1nc1cc(F)c(Cl)cc1c2CNC(=O)[C@H](O)C1CC1]

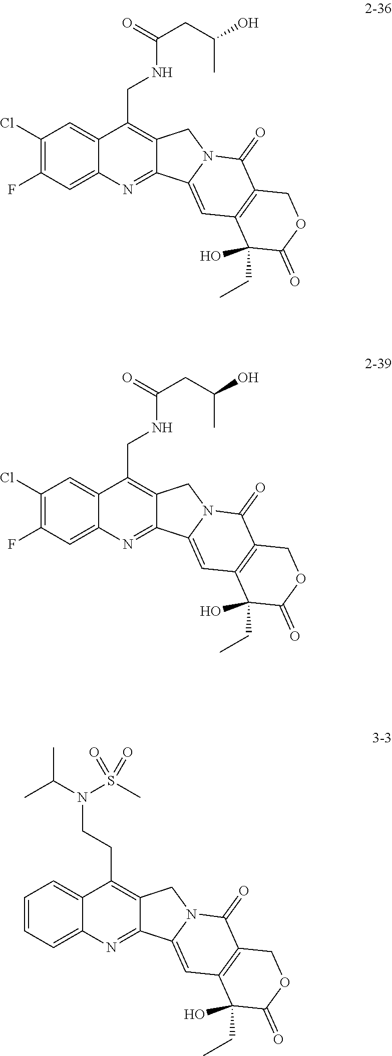 [SMILES: CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1ccccc1c2CCN(C(C)C)S(C)(=O)=O]

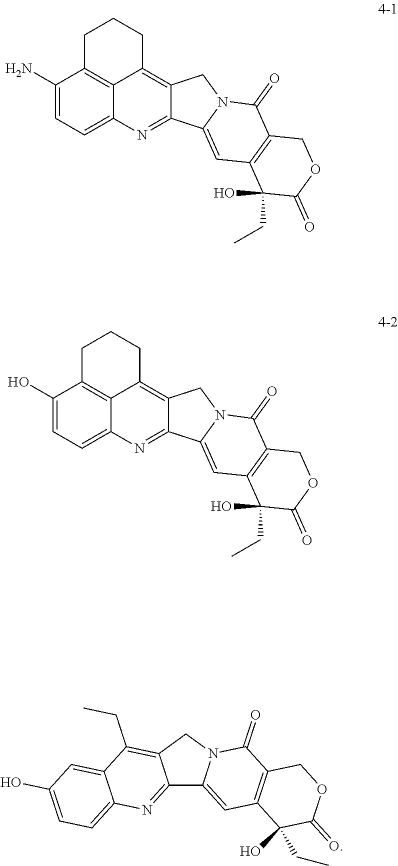 [SMILES: CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3 ; CCc1c2c(nc3ccc(O)cc13)-c1cc3c(c(=O)n1C2)COC(=O)[C@]3(O)CC]

In some embodiments, the cytotoxic drug is selected from the group consisting of the following compounds or isotopically labeled compounds thereof:

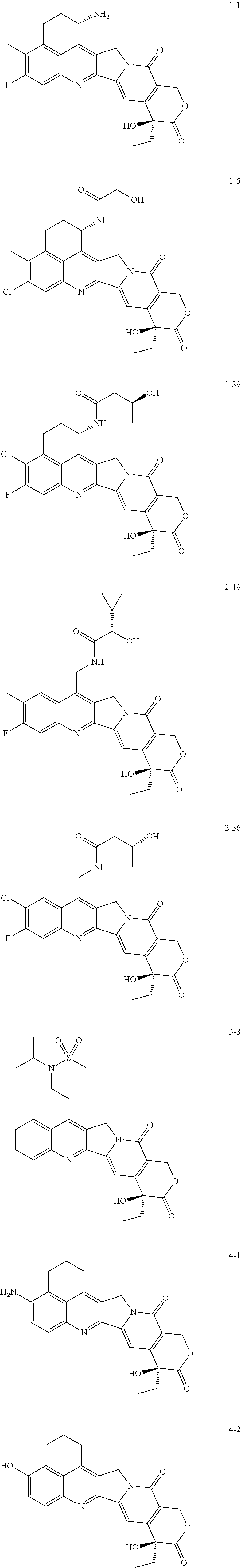

In some embodiments, the cytotoxic drug is selected from the group consisting of the following compounds or isotopically labeled compounds thereof:

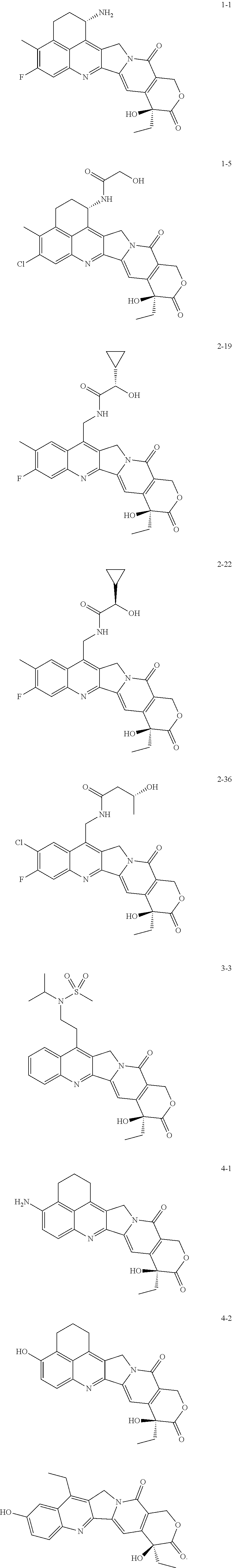

In some embodiments, the cytotoxic drug is selected from the group consisting of the following compounds or isotopically labeled compounds thereof:

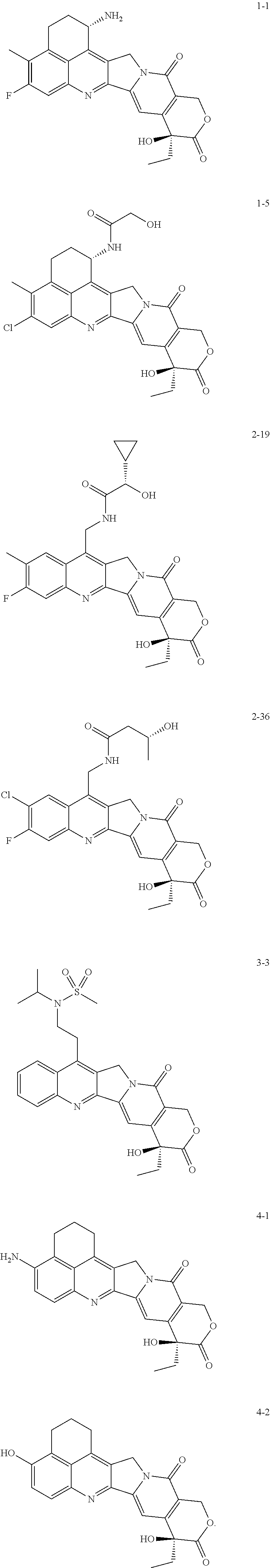

In some embodiments, the cytotoxic drug is selected from the group consisting of the following compounds or isotopically labeled compounds thereof:

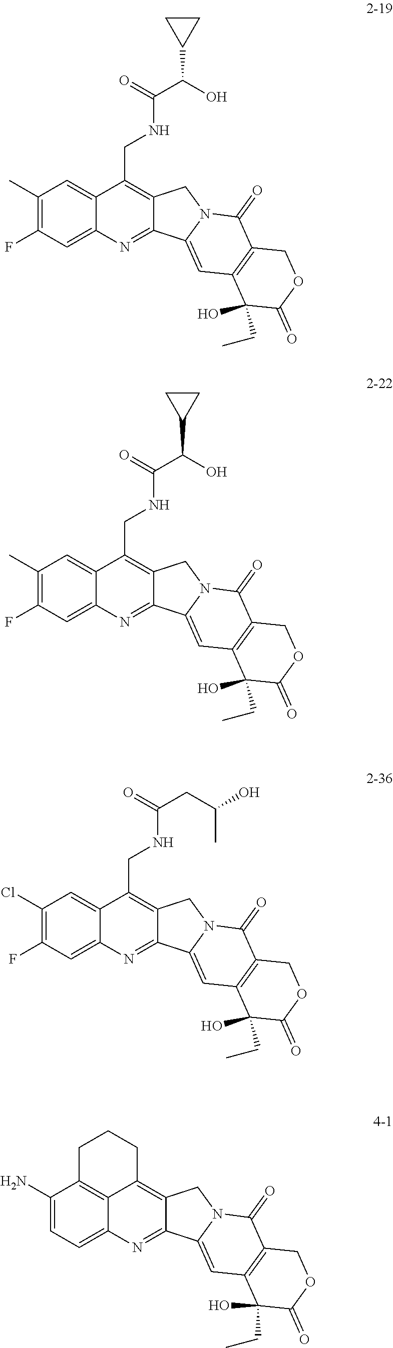 [SMILES: CC[C@@]1(O)C(=O)OCc2c1cc1n(c2=O)Cc2c-1nc1cc(F)c(C)cc1c2CNC(=O)[C@@H](O)C1CC1 ; CC[C@@]1(O)C(=O)OCc2c1cc1n(c2=O)Cc2c-1nc1cc(F)c(C)cc1c2CNC(=O)[C@H](O)C1CC1 ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1ccc(N)c3c1c2CCC3]

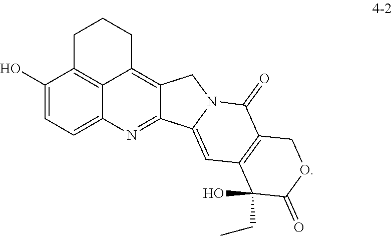 [SMILES: CC[C@@]1(O)C(=O)OCc2c1cc1n(c2=O)Cc2c-1nc1ccc(O)c3c1c2CCC3]

In some embodiments, the cytotoxic drug is selected from the group consisting of the following compounds or isotopically labeled compounds thereof:

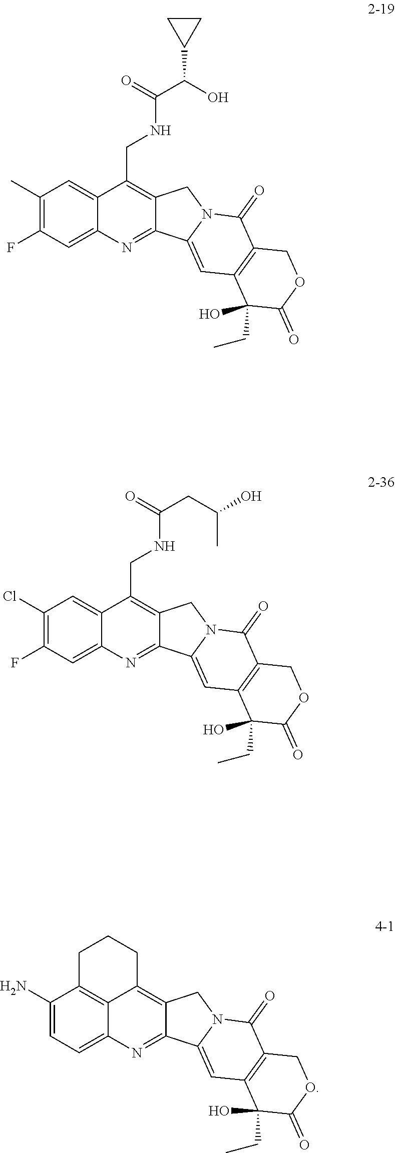 [SMILES: CC[C@@]1(O)C(=O)OCc2c1cc1n(c2=O)Cc2c-1nc1cc(F)c(C)cc1c2CNC(=O)[C@@H](O)C1CC1 ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1ccc(N)c3c1c2CCC3]

In some embodiments, the structure of the cytotoxic drug fragment D is represented by the following Formula III-A, Formula III-B or Formula III-C:

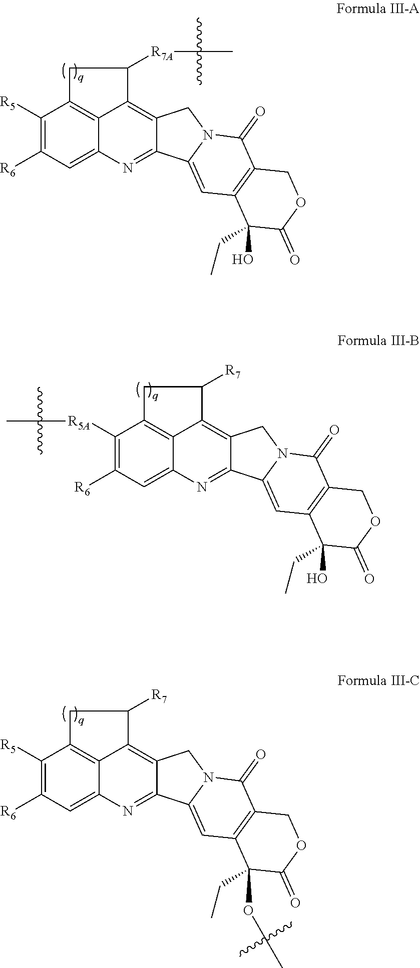

wherein, R_{5A} is selected from the group consisting of —O— or —N—; R_{7A} is selected from the group consisting of a chemical bond, —O—, —NH—, —N(C₁₋₆ alkyl)-, and —NH—CO—(C₁₋₆ alkylene)-O—; the C₁₋₆ alkyl is optionally further substituted with one or more substituents selected from the group consisting of halogen, hydroxyl, C₁₋₆ haloalkyl, and C₃₋₆ cycloalkyl;
R₅ to R₇, q are as described above.

In some embodiments, R_{7A} is selected from the group consisting of a chemical bond, —O—, —NH—, —N(C₁₋₄ alkyl)-, and —NH—CO—(C₁₋₄ alkylene)-O—; the C₁₋₄ alkyl is optionally further substituted with one or more substituents selected from the group consisting of halogen, hydroxyl, C₁₋₄ haloalkyl, and C₃₋₆ cycloalkyl. In some embodiments, R_{7A} is selected from the group consisting of a chemical bond, —O—, —NH—, —N(CH₃)—, —N(CH₂CH₃)—, —N(CH(CH₃)₂)—, —NH—CO—CH₂—O—, —NH—CO—CH₂CH₂—O—, —NH—CO—CH₂(CH₃)—O—, —NH—CO—CH₂CH(CH₃)—O—. In some embodiments, R_{7A} is selected from the group consisting of —NH—, —NH—CO—CH₂—O—, —NH—CO—CH₂CH(CH₃)—O—.

In some embodiments, the cytotoxic drug fragment D is selected from the group represented by the following Formula IV-A or Formula IV-B

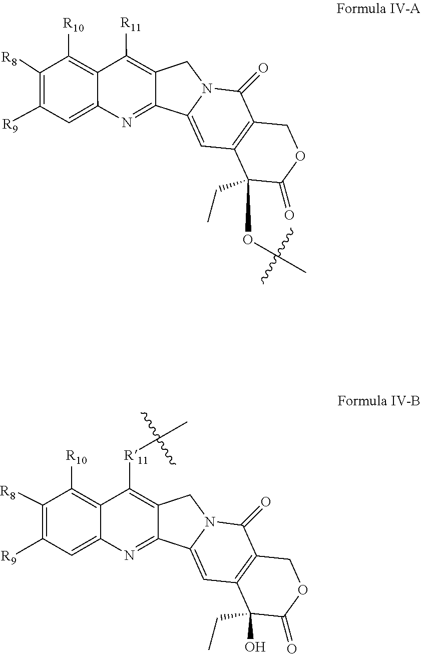

wherein, R₈ to R₁₁ are as described above; R′₁₁ is —C₁₋₄ alkylene-NH(C₁₋₆ alkylene)-O— or —C₁₋₄ alkylene-N(C₁₋₆ alkyl)(C₁₋₆ alkylene)-O—; the C₁₋₄ alkylene, C₁₋₆ alkylene or C₁₋₆ alkyl is optionally further substituted with one or more substituents selected from the group consisting of halogen, hydroxyl, C₁₋₆ haloalkyl, C₁₋₆ alkoxy, C₃₋₆ cycloalkyl.

In some embodiments, D is selected from the group consisting of:

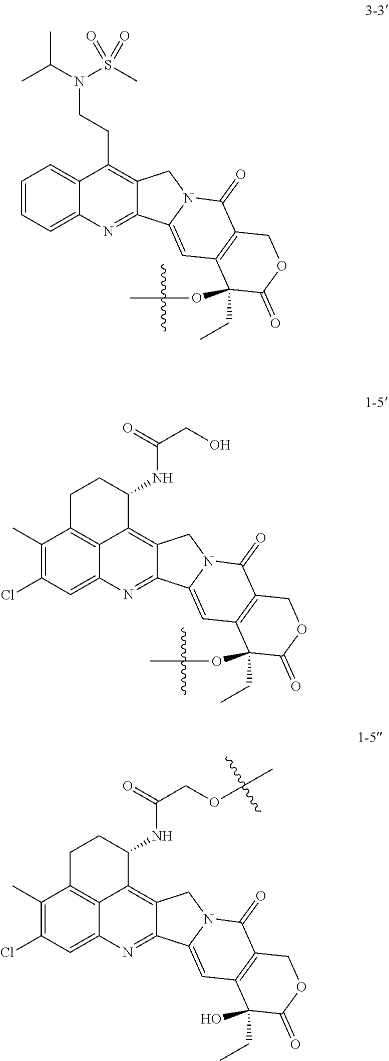 [SMILES: *O[C@]1(CC)C(=O)OCc2c1cc1n(c2=O)Cc2c-1nc1ccccc1c2CCN(C(C)C)S(C)(=O)=O ; *O[C@]1(CC)C(=O)OCc2c1cc1n(c2=O)Cc2c-1nc1cc(Cl)c(C)c3c1c2[C@@H](NC(=O)CO)CC3 ; *OCC(=O)N[C@H]1CCc2c(C)c(Cl)cc3nc4c(c1c23)Cn1c-4cc2c(c1=O)COC(=O)[C@]2(O)CC]

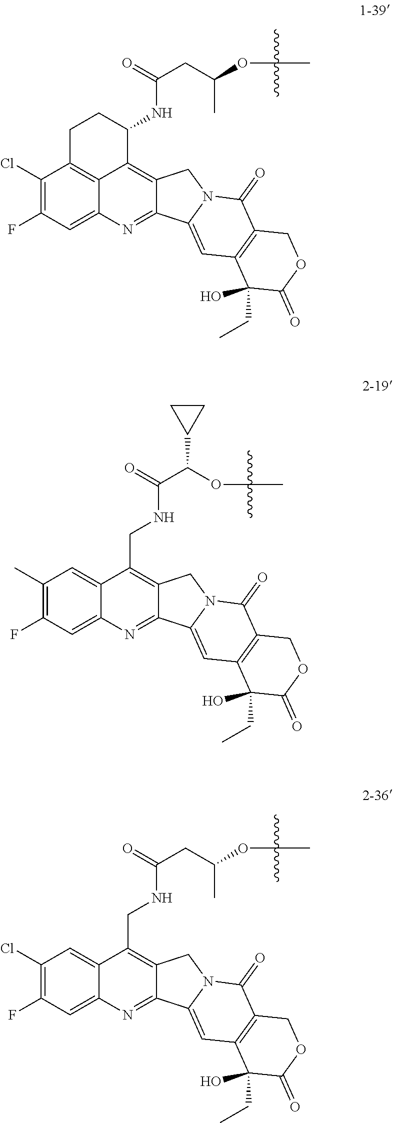 [SMILES: *O[C@@H](C)CC(=O)N[C@H]1CCc2c(Cl)c(F)cc3nc4c(c1c23)Cn1c-4cc2c(c1=O)COC(=O)[C@]2(O)CC ; *O[C@H](C(=O)NCc1c2c(nc3cc(F)c(C)cc13)-c1cc3c(c(=O)n1C2)COC(=O)[C@]3(O)CC)C1CC1 ; *O[C@H](C)CC(=O)NCc1c2c(nc3cc(F)c(Cl)cc13)-c1cc3c(c(=O)n1C2)COC(=O)[C@]3(O)CC]

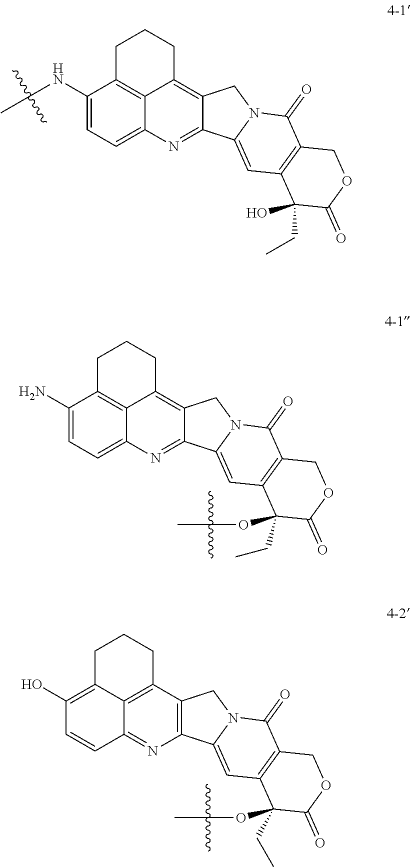 [SMILES: *Nc1ccc2nc3c(c4c2c1CCC4)Cn1c-3cc2c(c1=O)COC(=O)[C@]2(O)CC ; *O[C@]1(CC)C(=O)OCc2c1cc1n(c2=O)Cc2c-1nc1ccc(O)c3c1c2CCC3 ; *O[C@]1(CC)C(=O)OCc2c1cc1n(c2=O)Cc2c-1nc1ccc(O)c3c1c2CCC3]

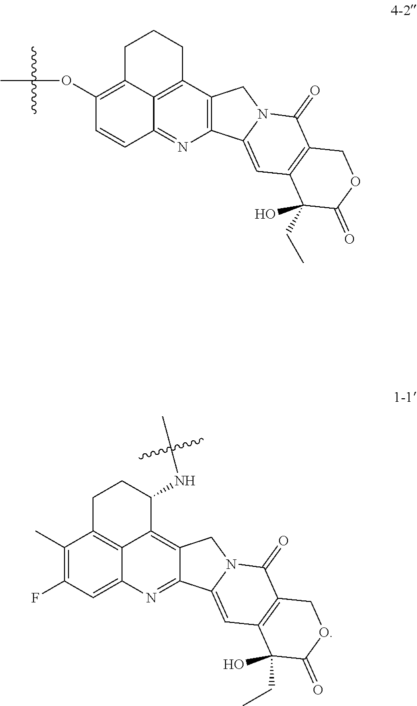 [SMILES: *Oc1ccc2nc3c(c4c2c1CCC4)Cn1c-3cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC]

In some embodiments, the compound or pharmaceutically acceptable salt thereof is selected from the group consisting of A-1 to A-204, B-1 to B-24 as shown below:

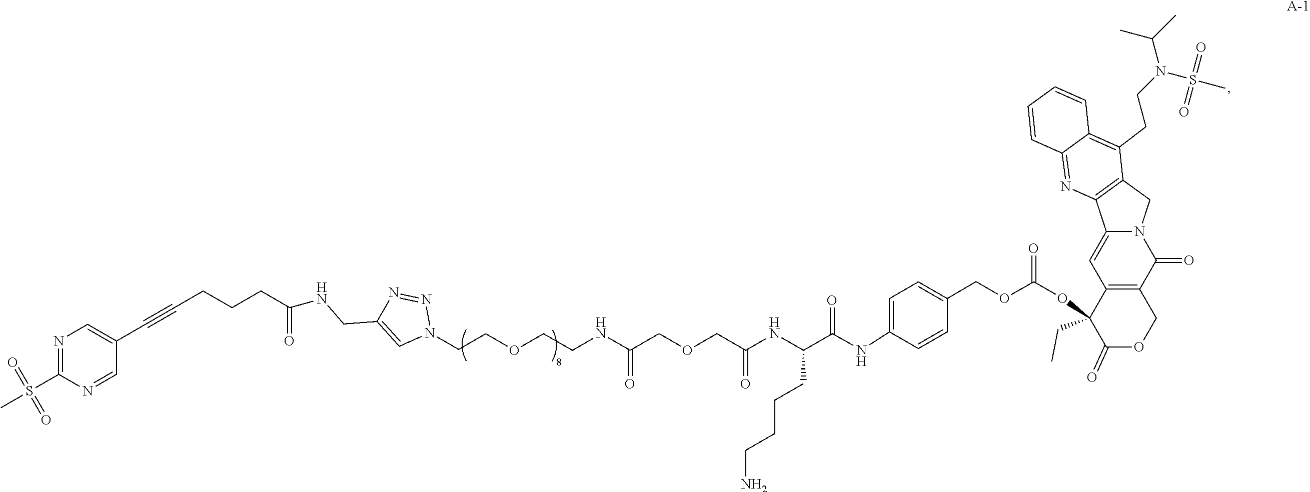

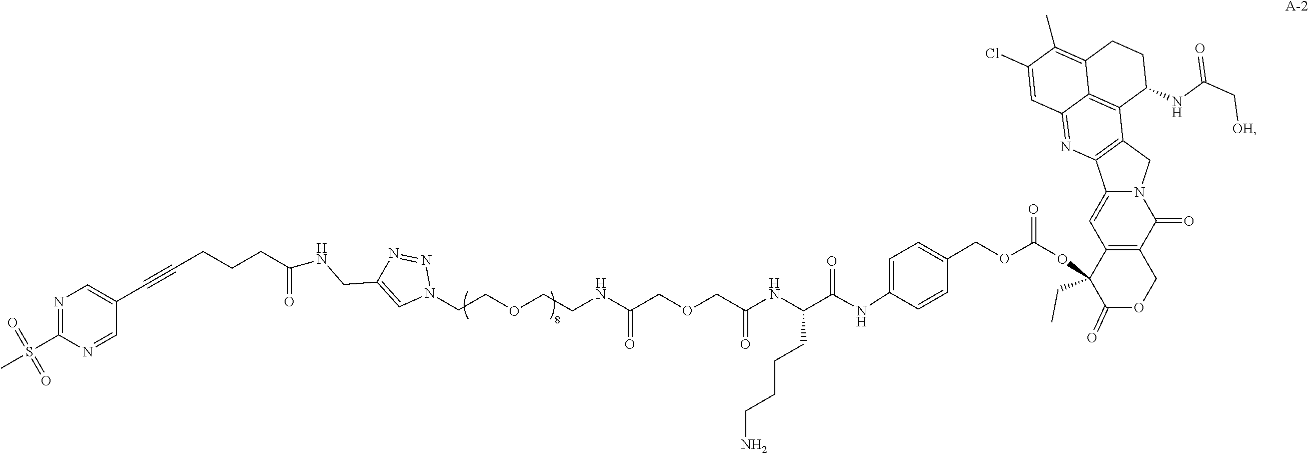

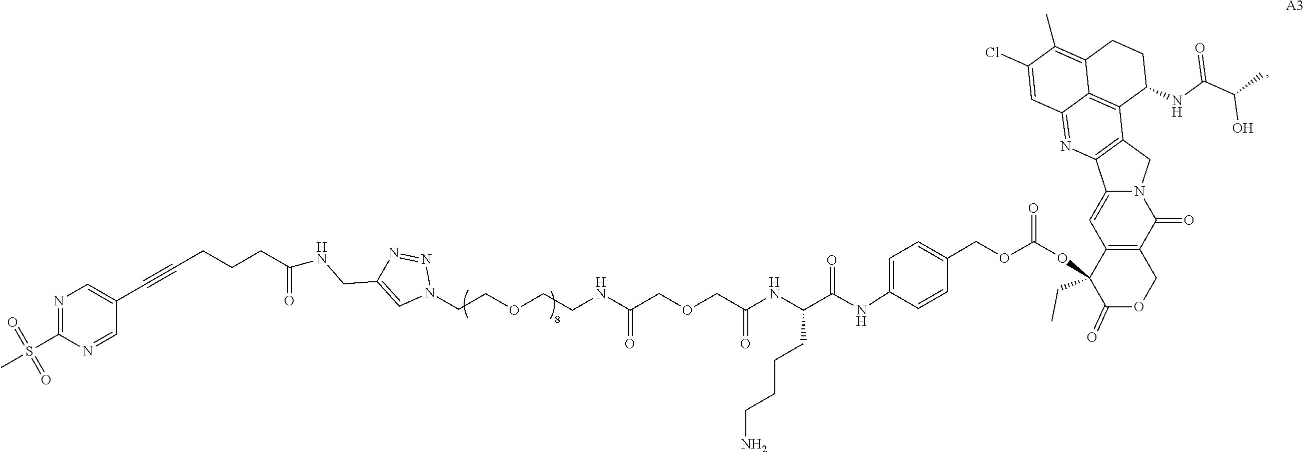

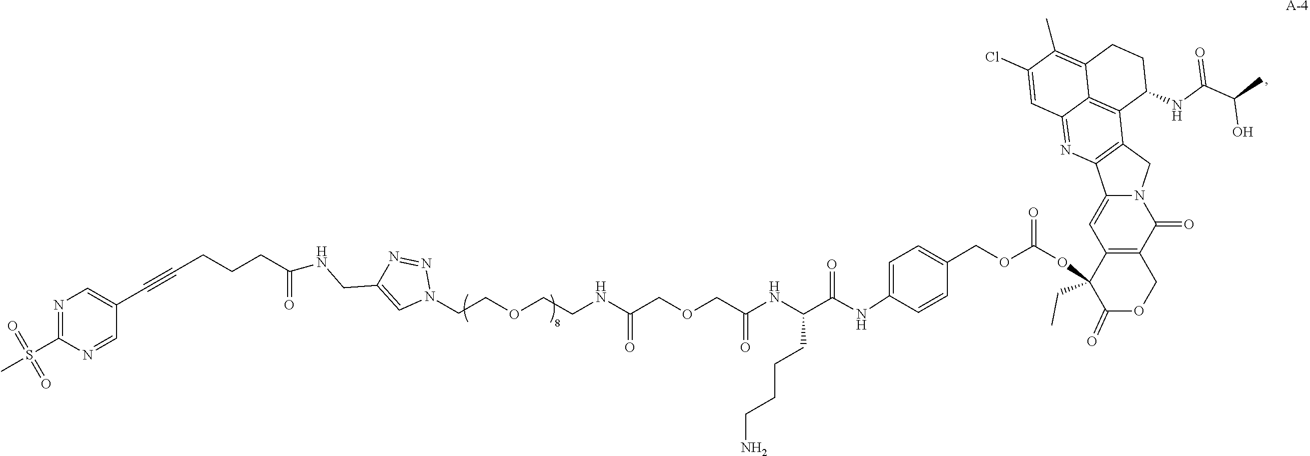

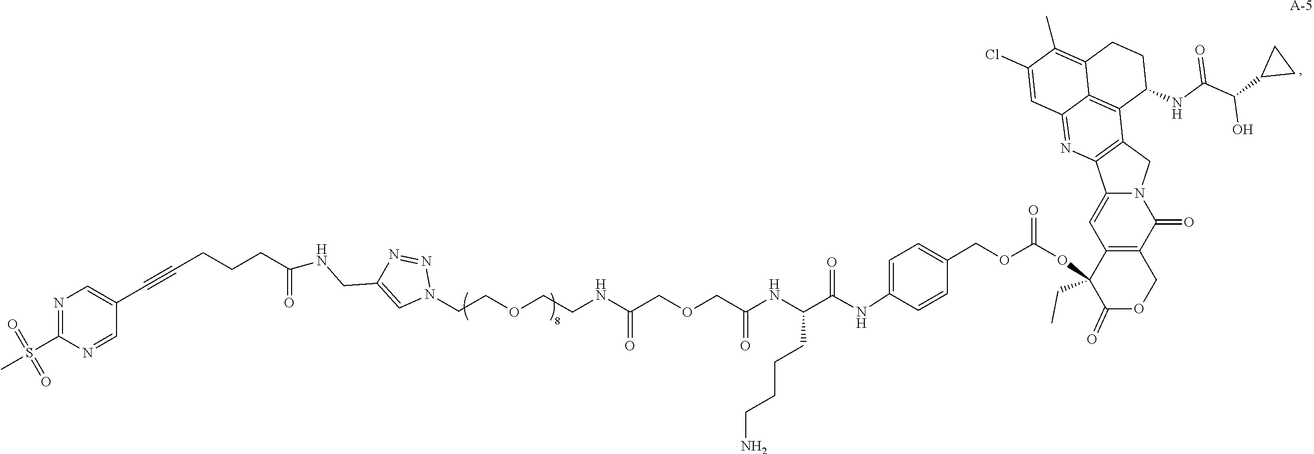

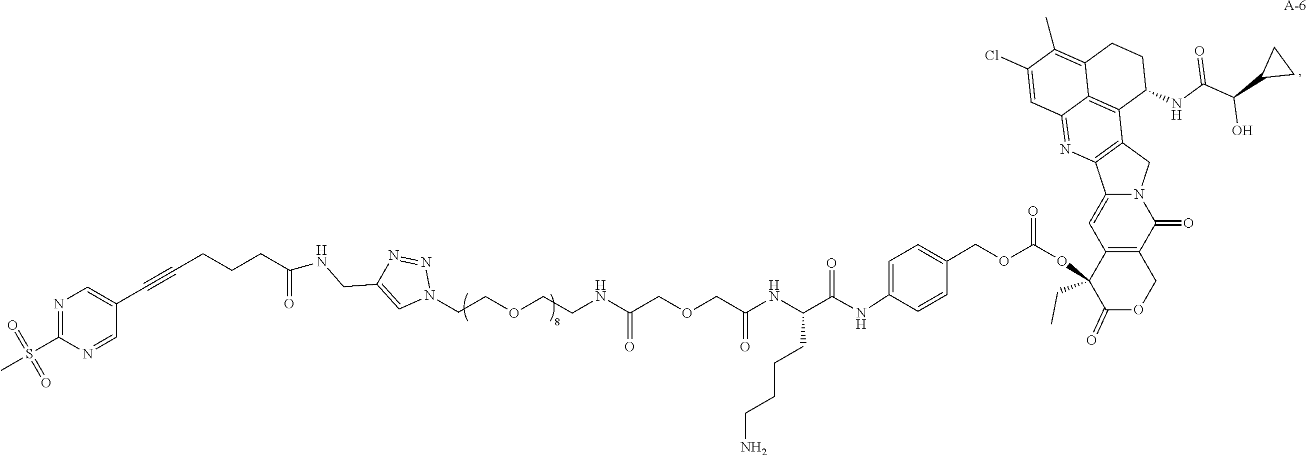

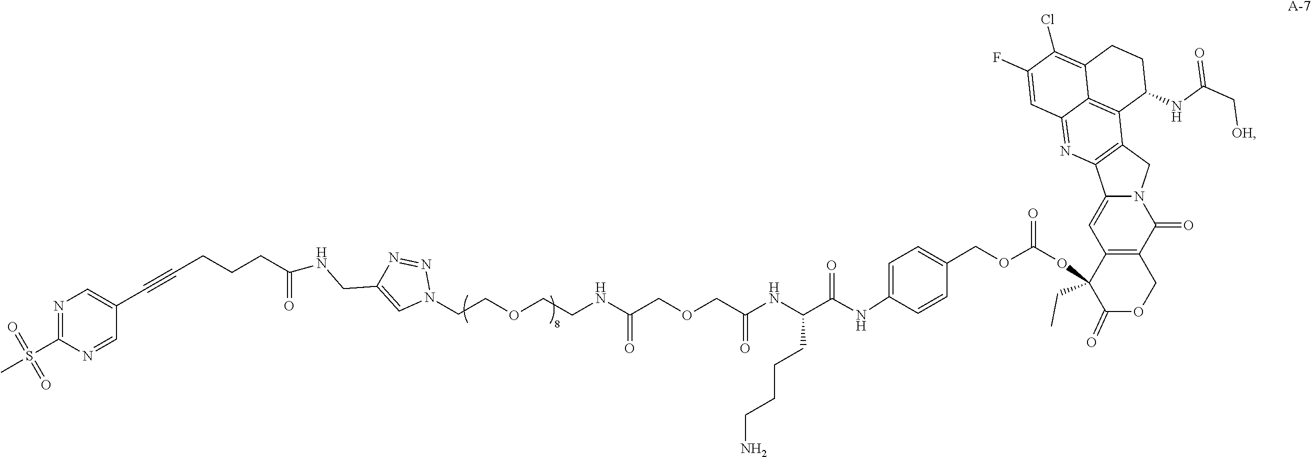

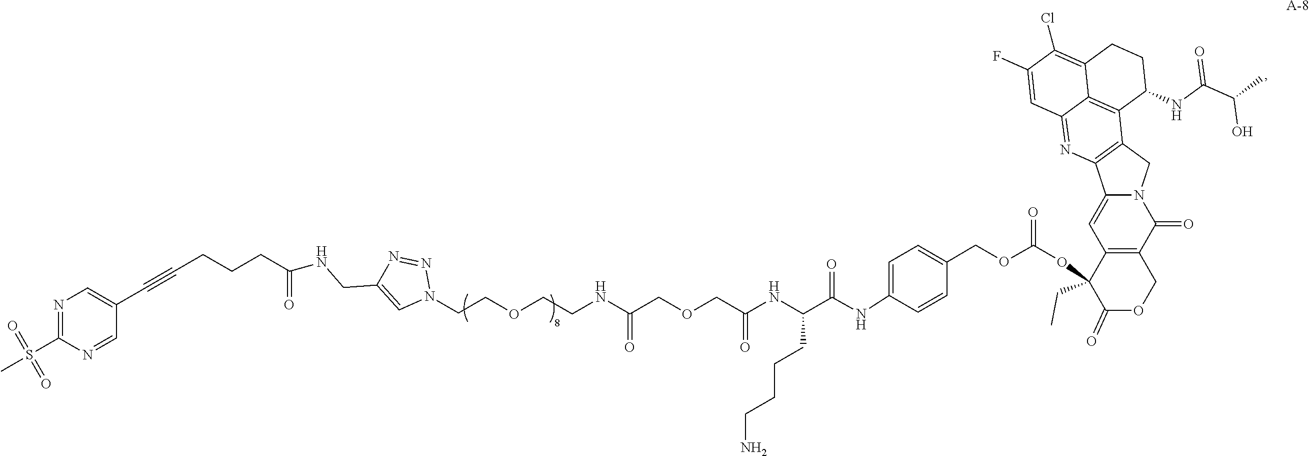

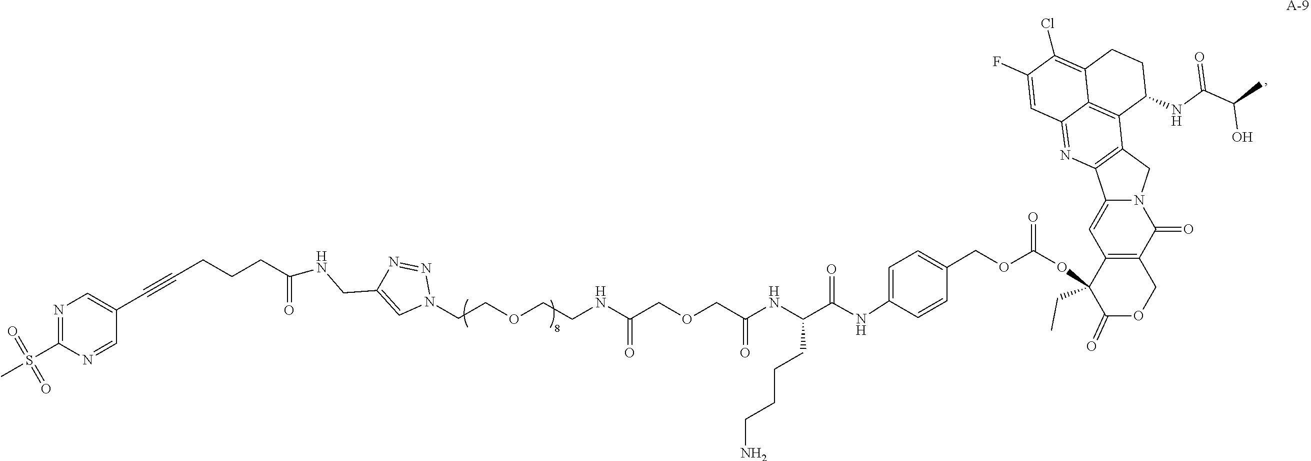

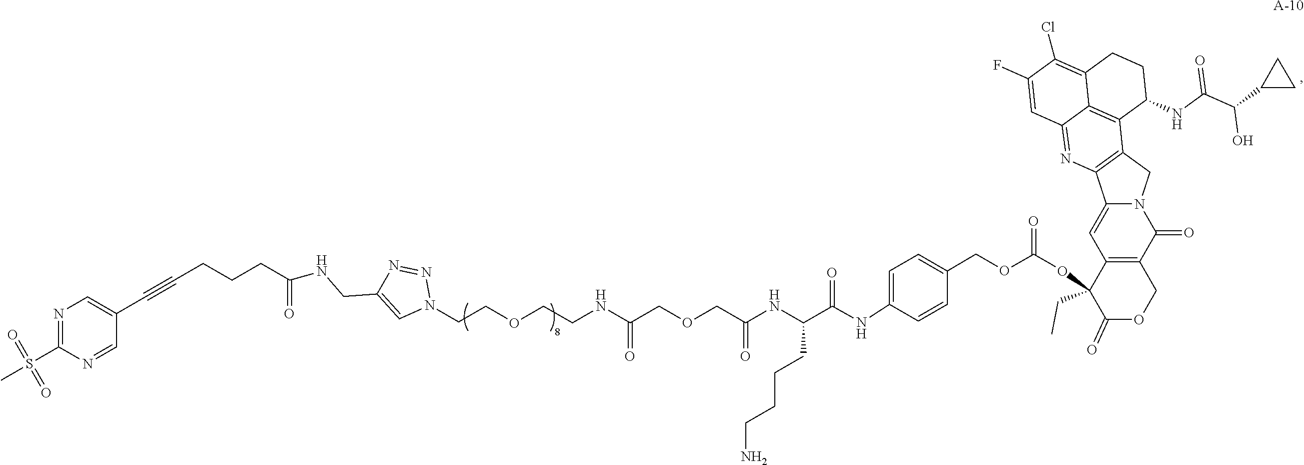

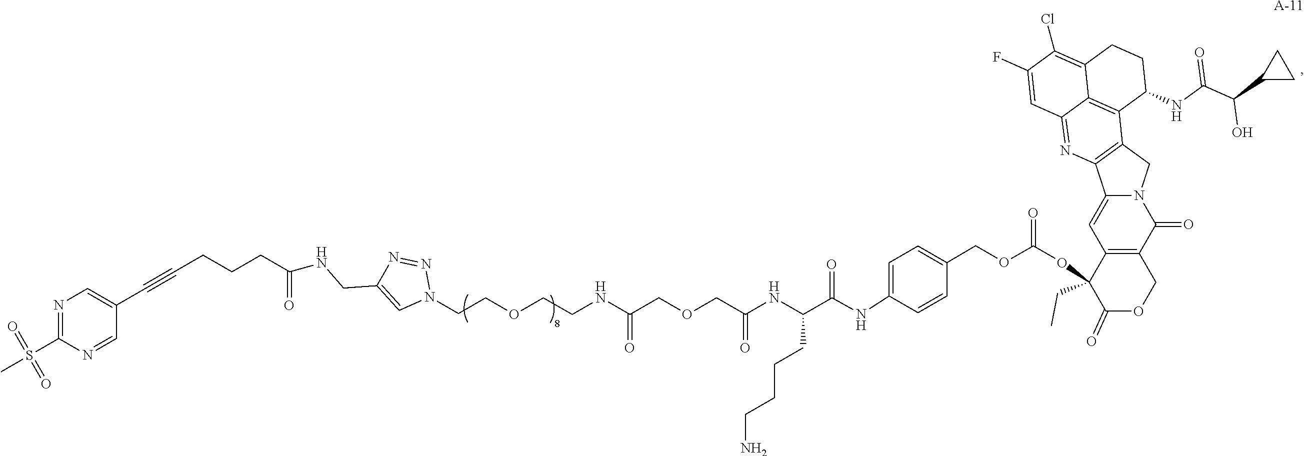

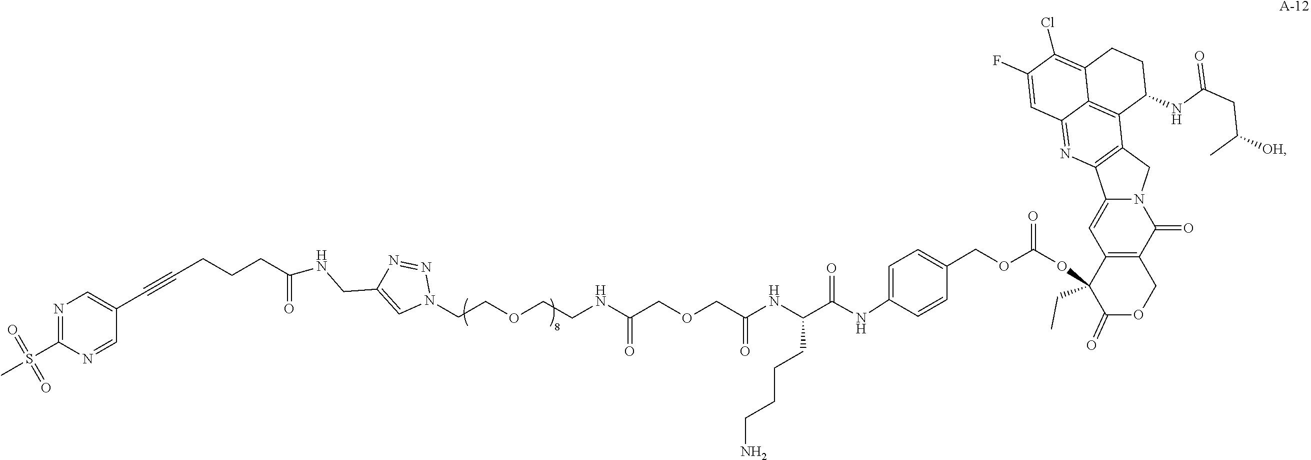

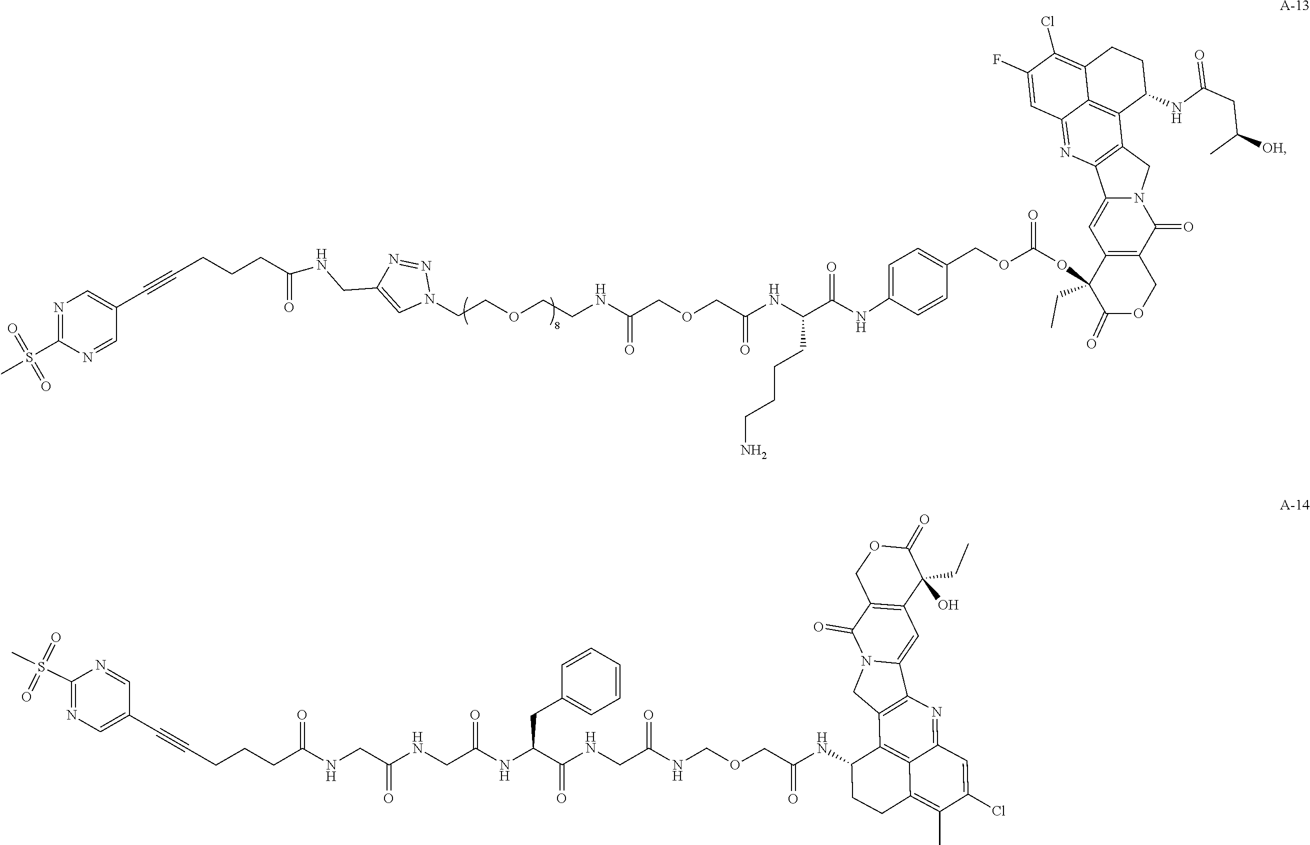

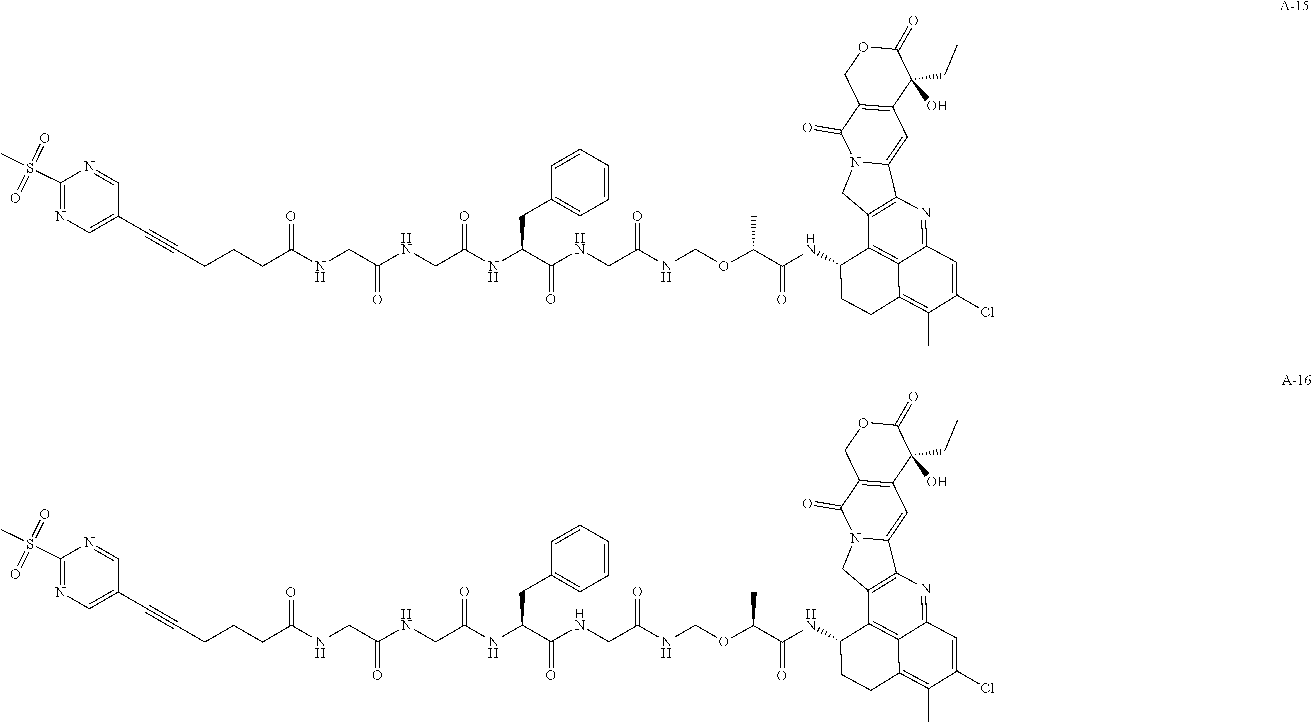

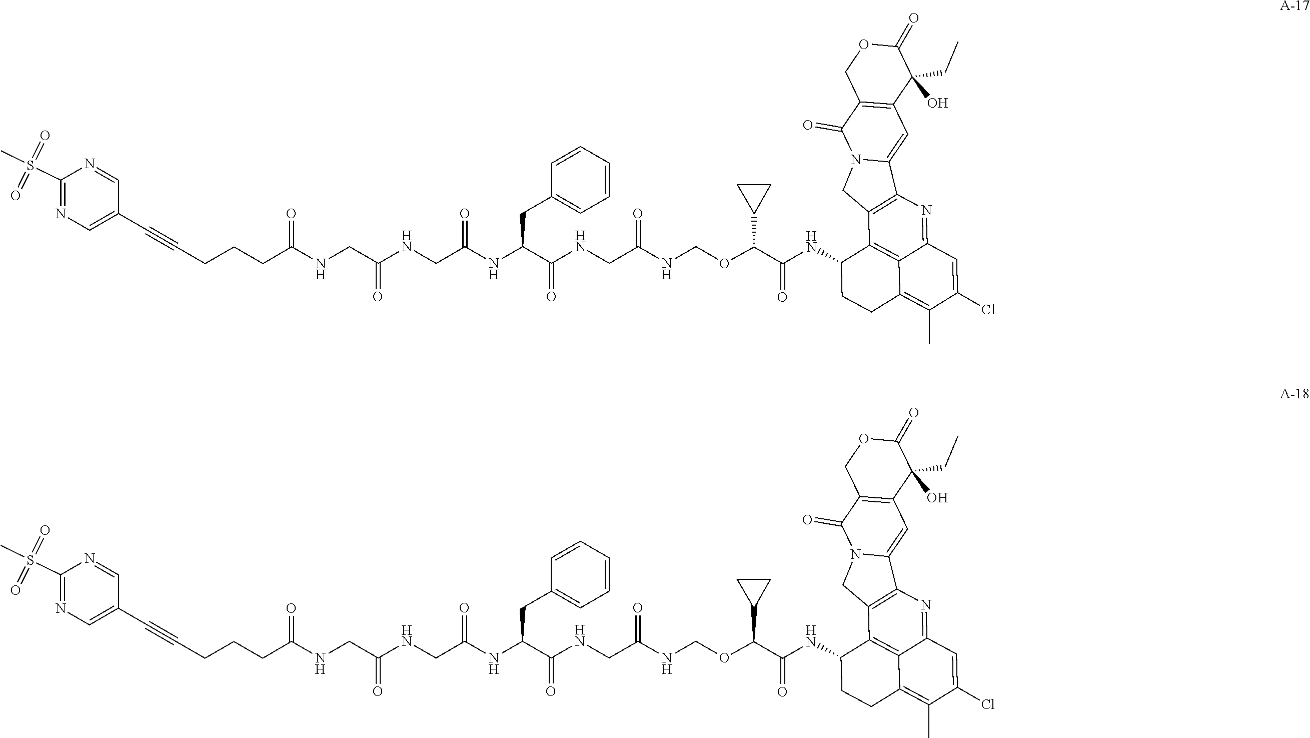

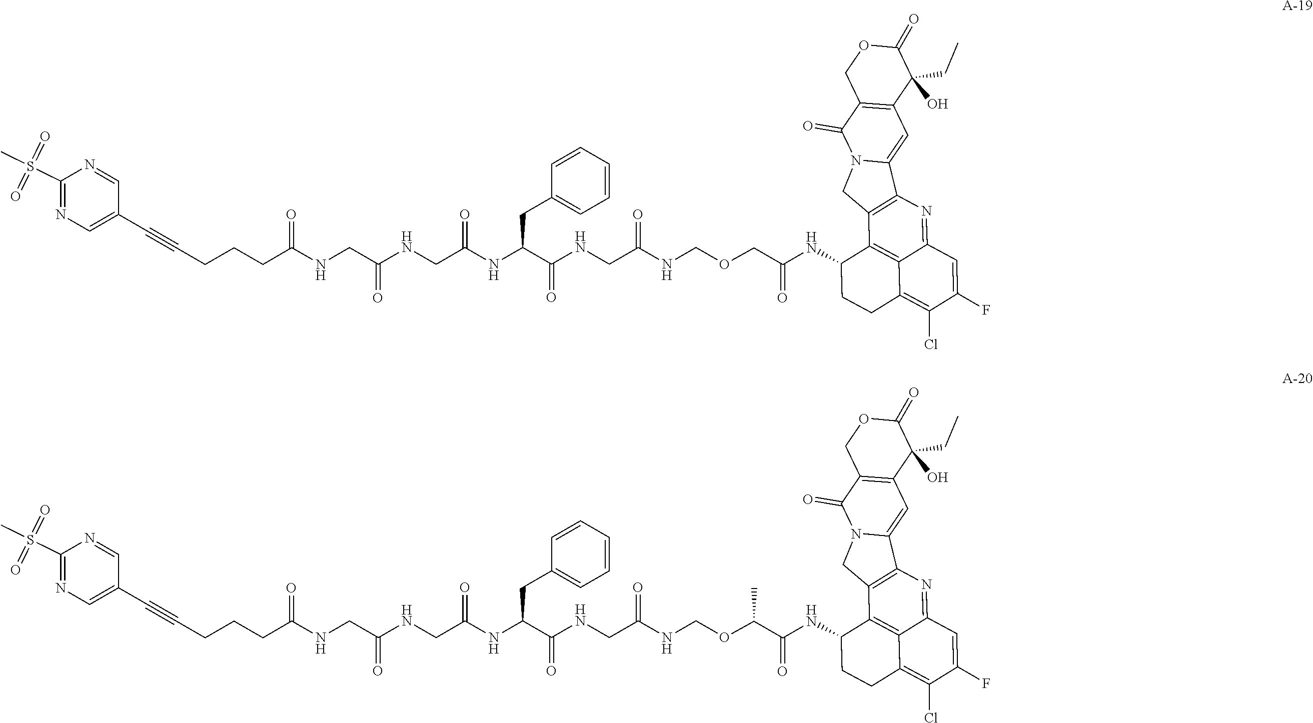

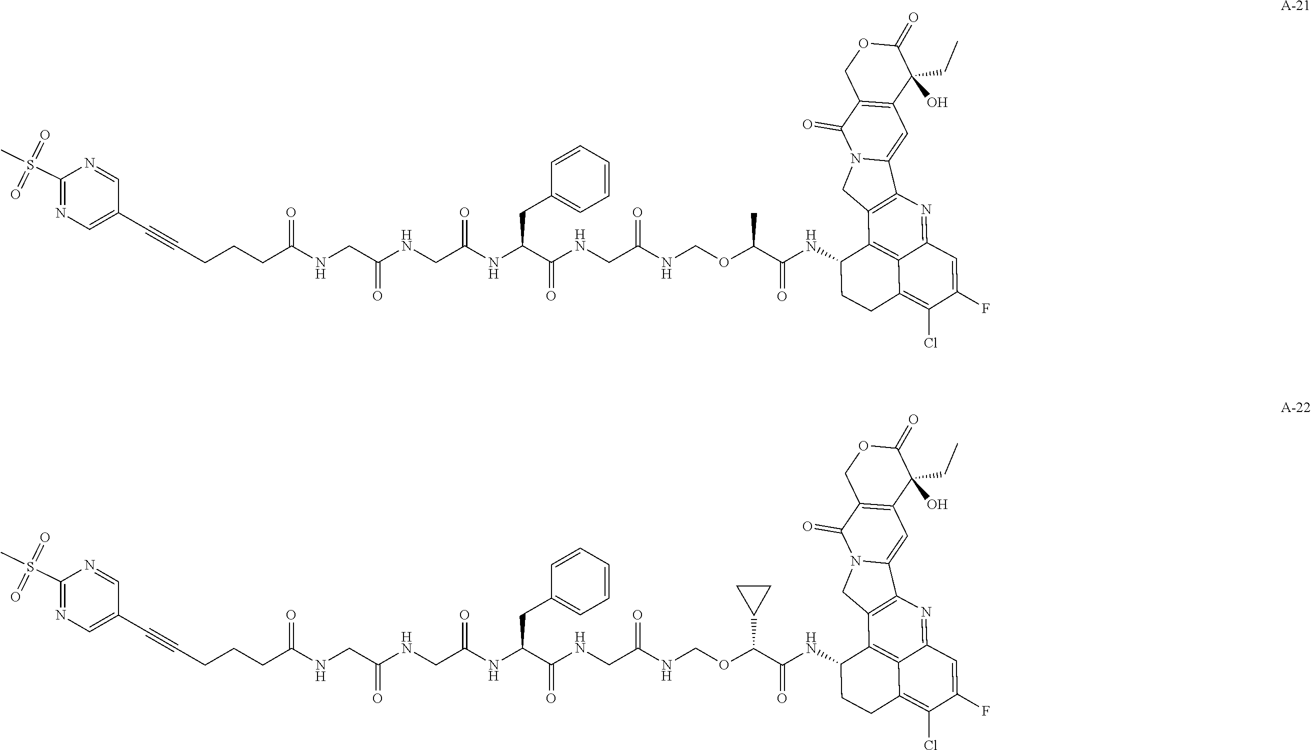

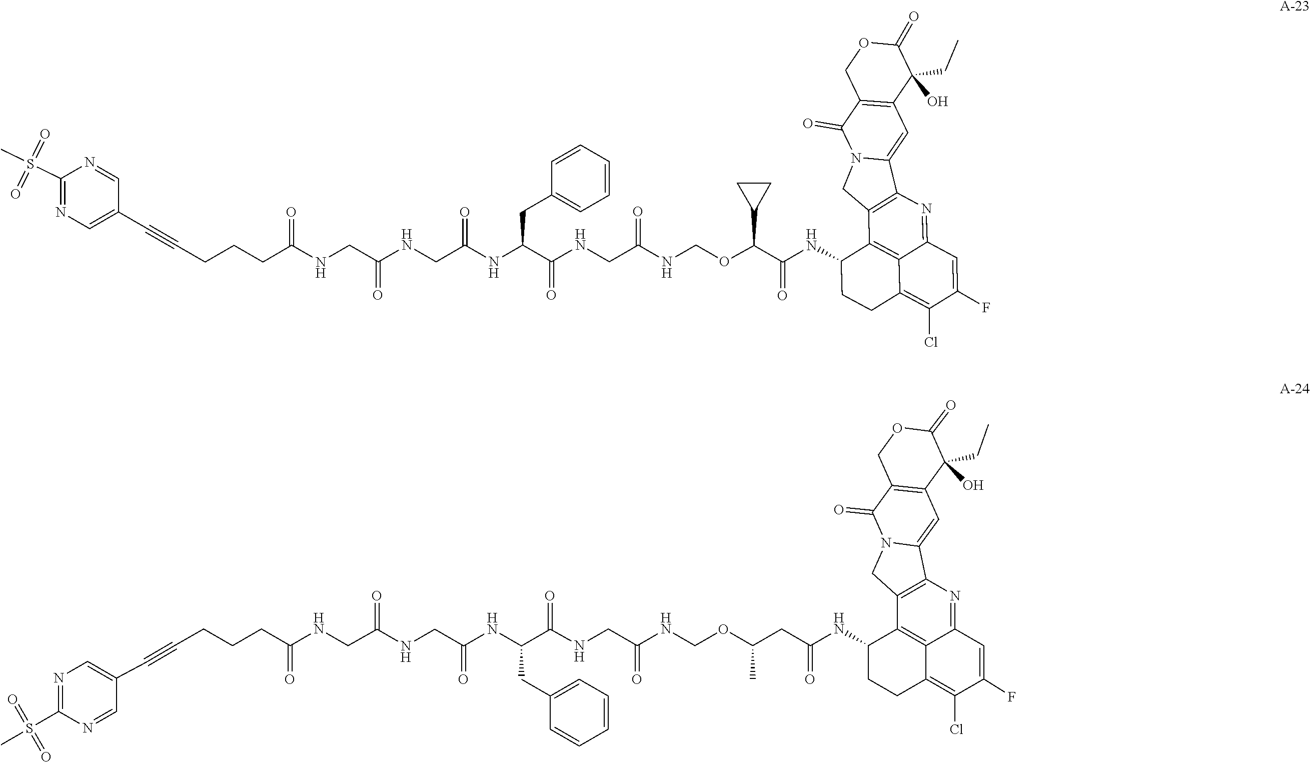

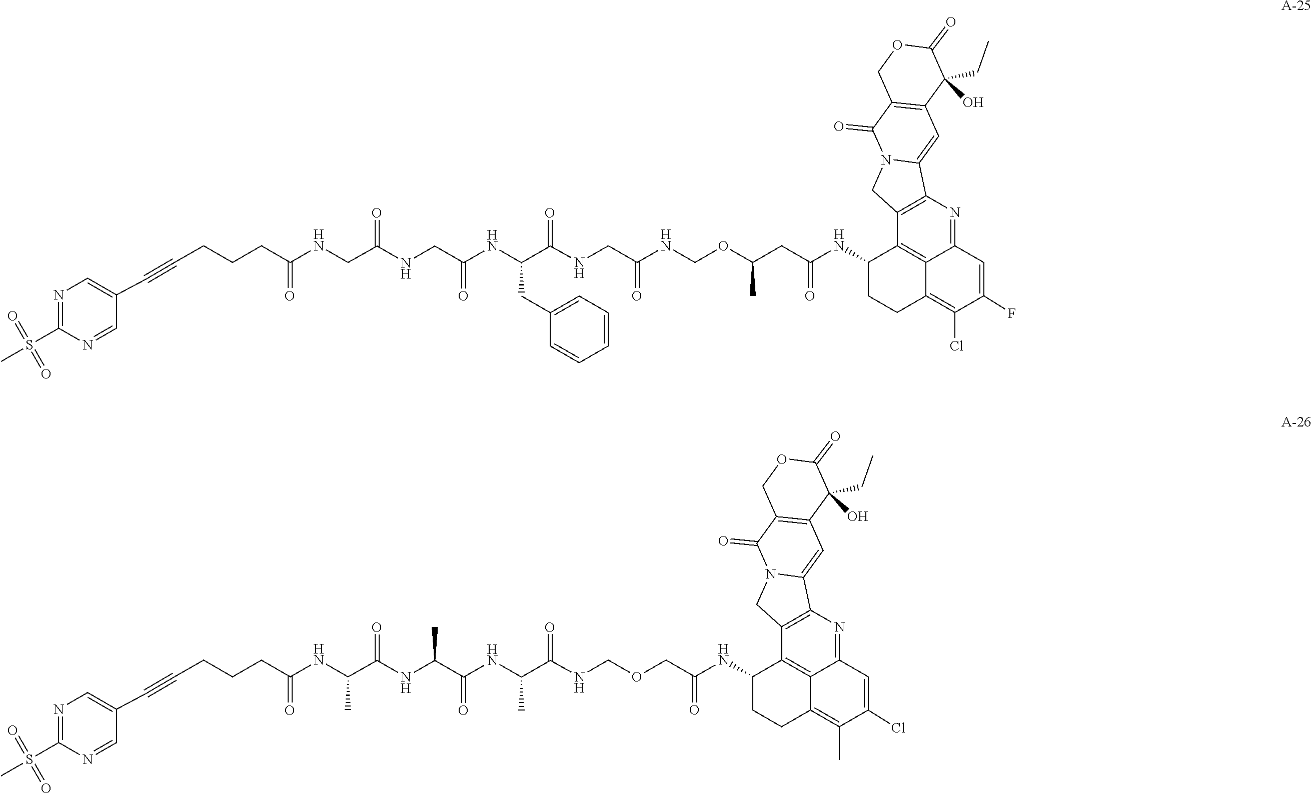

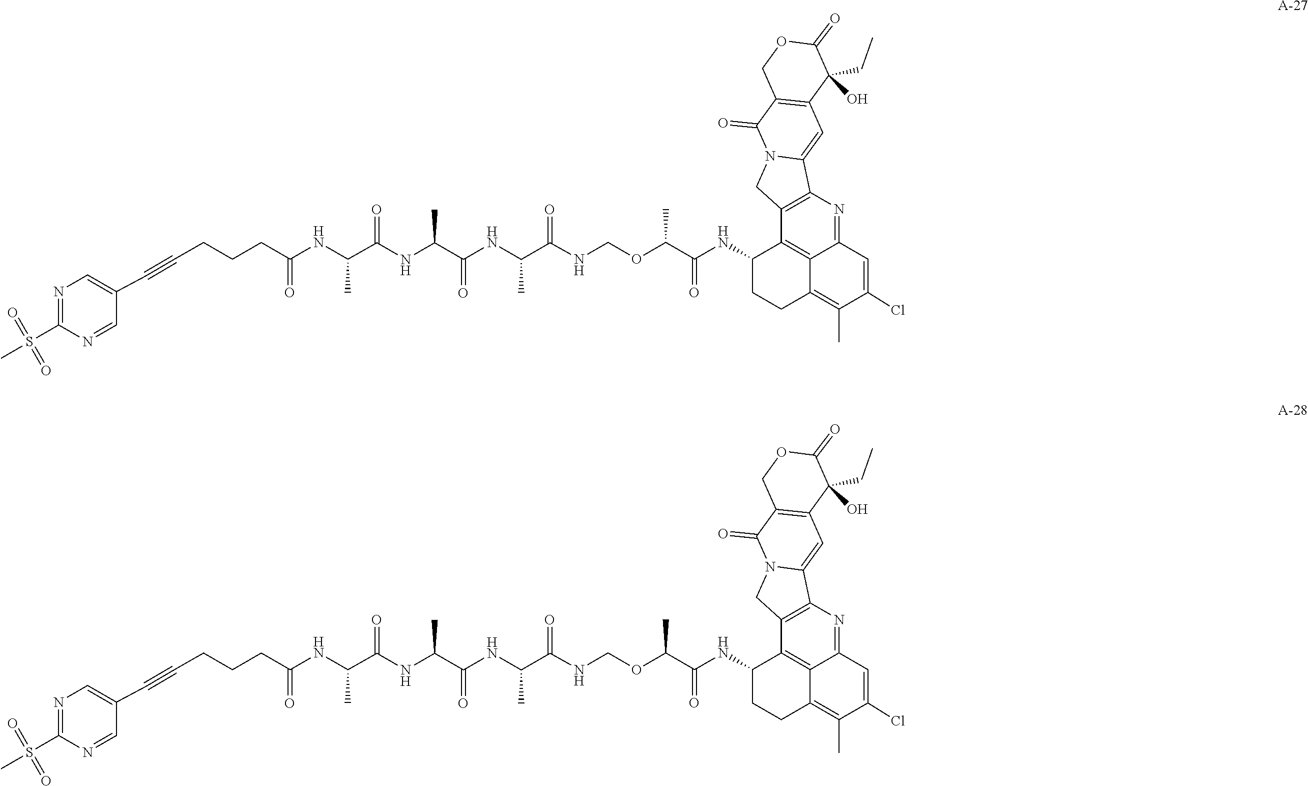

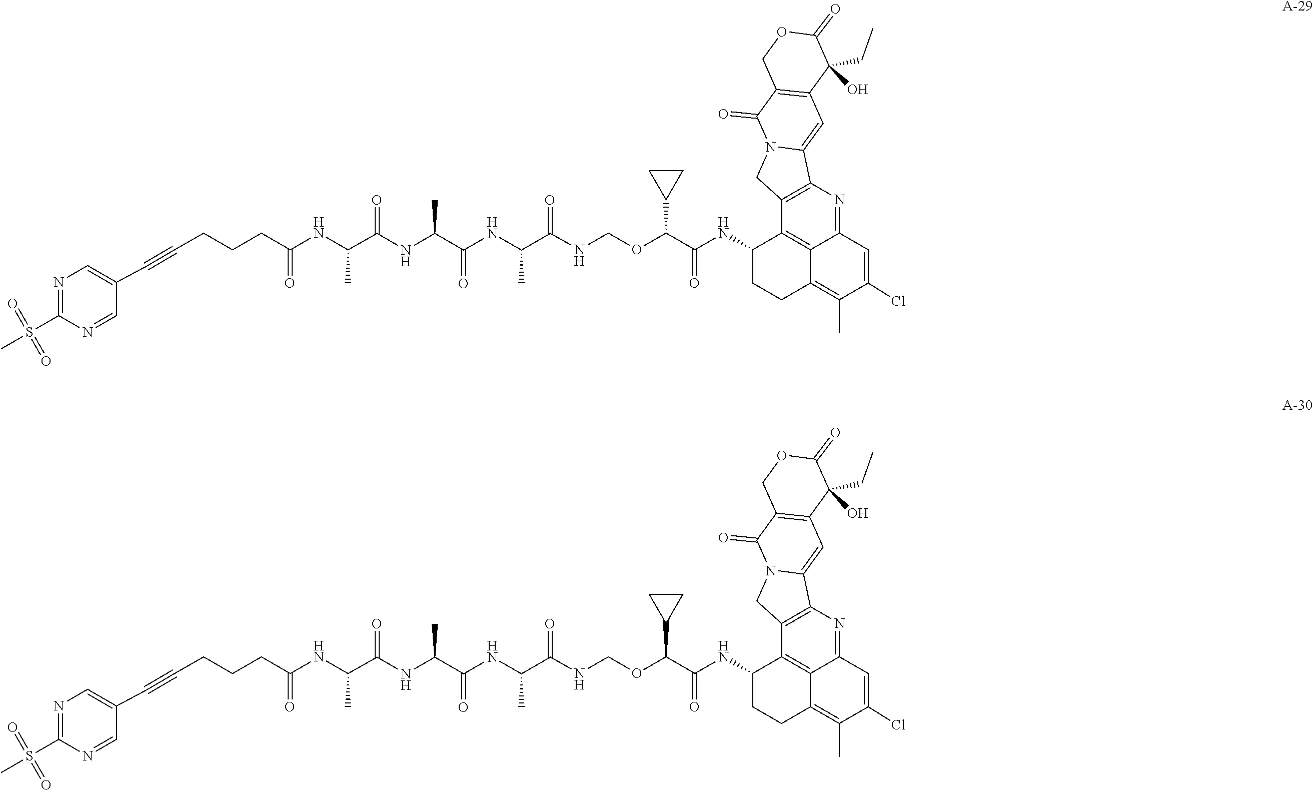

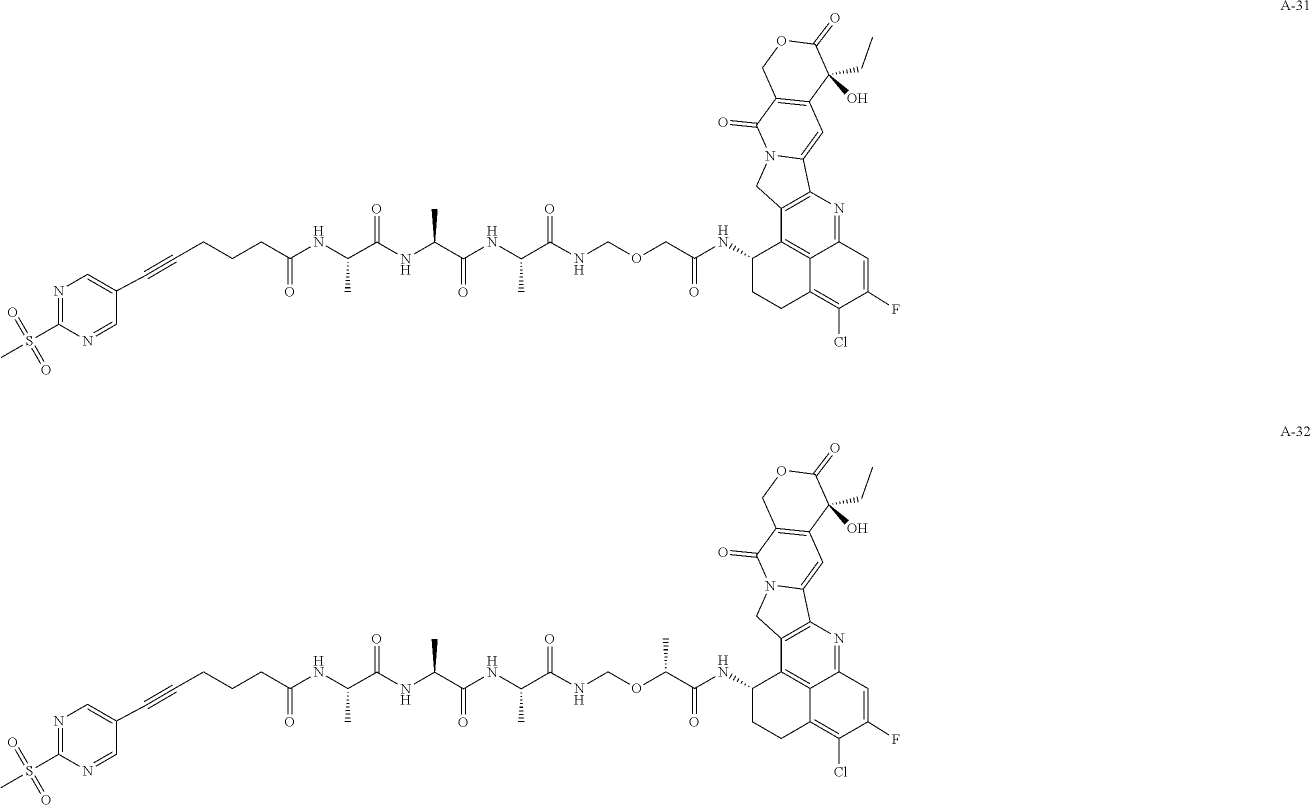

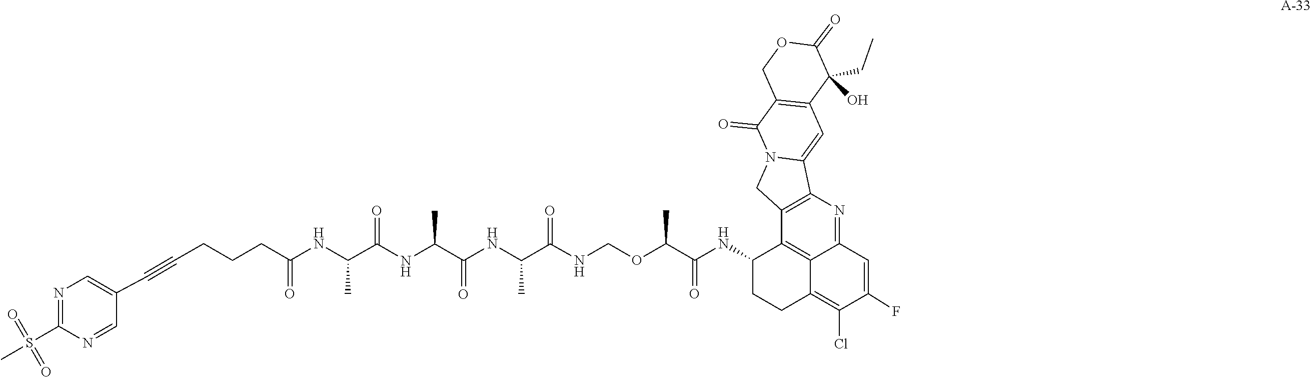

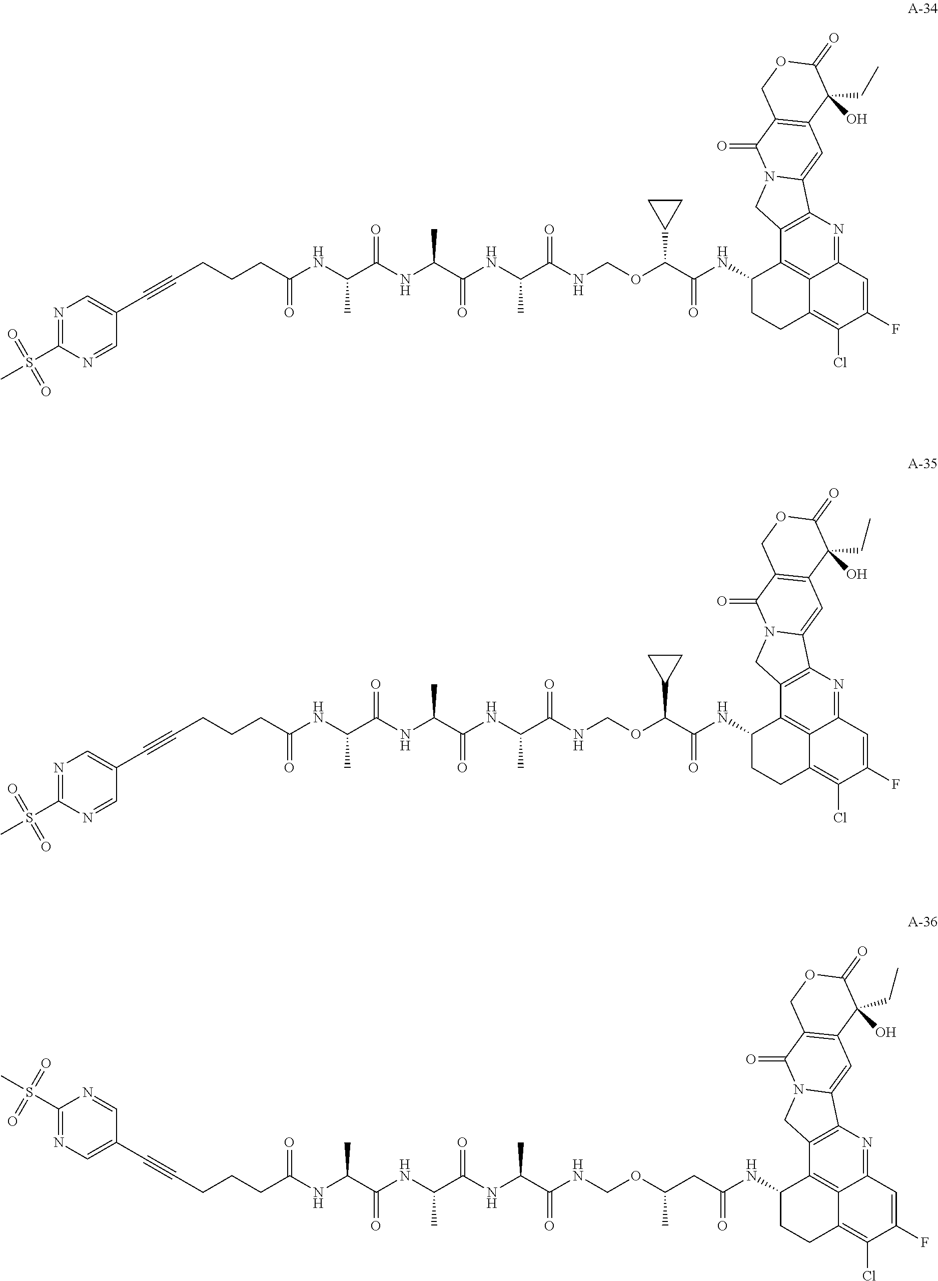

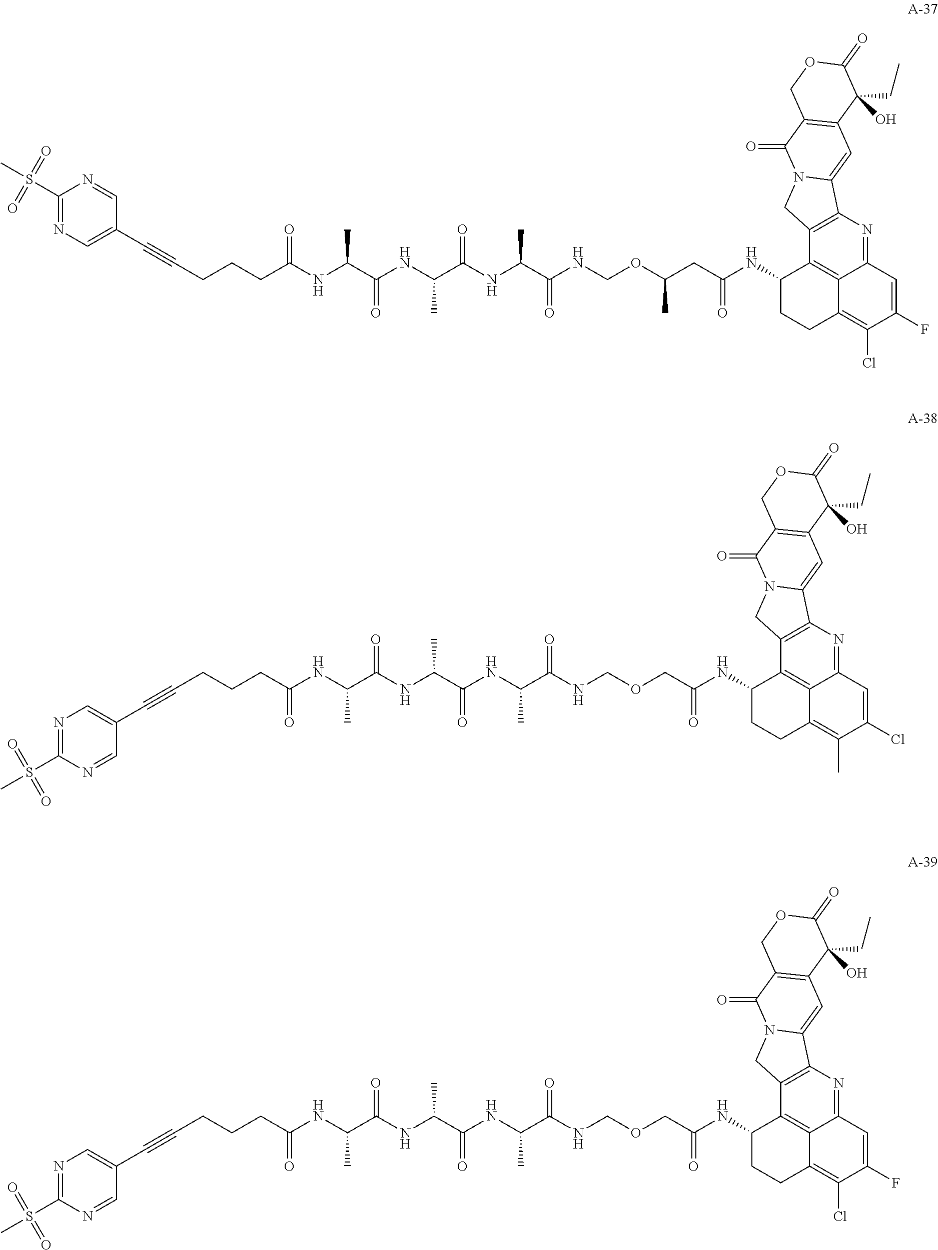

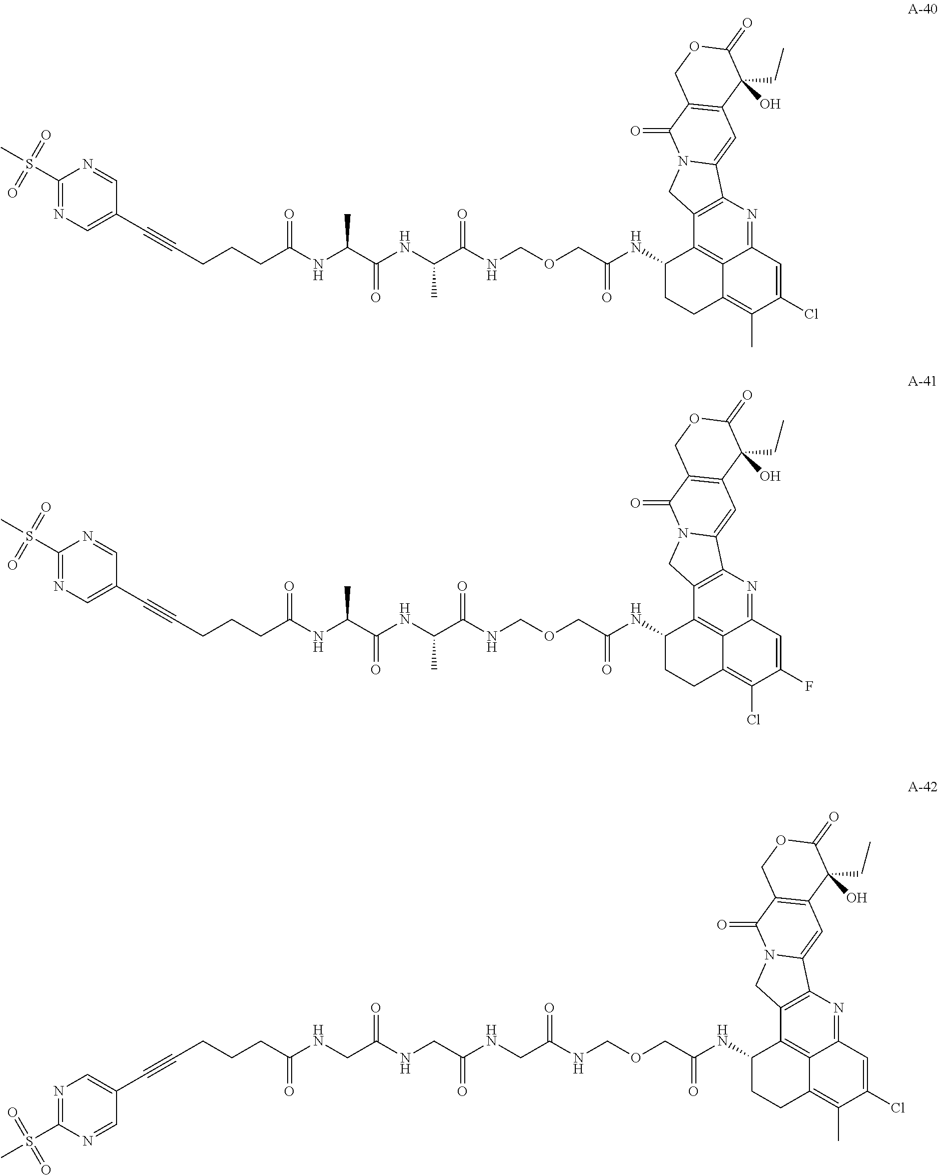

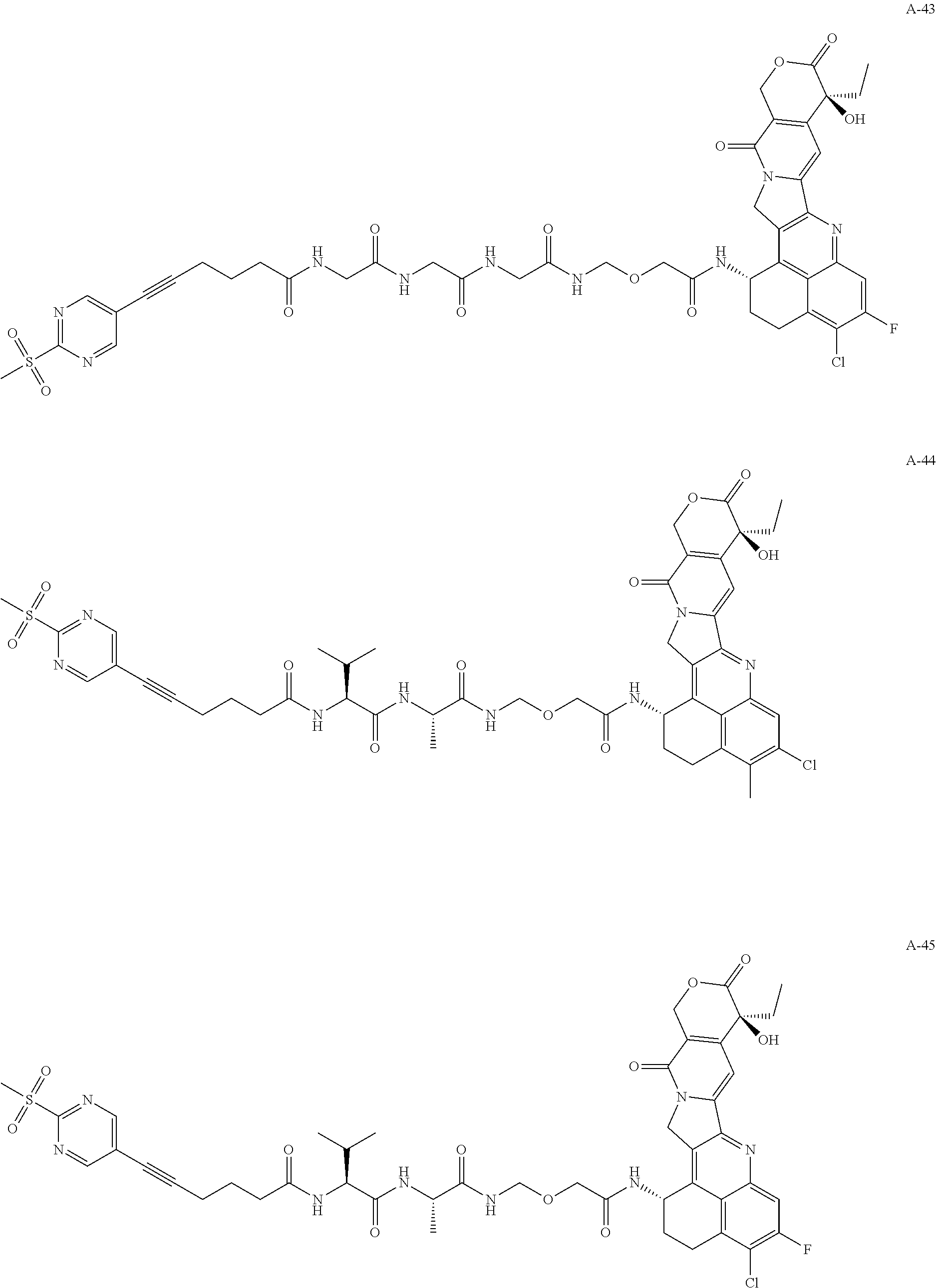

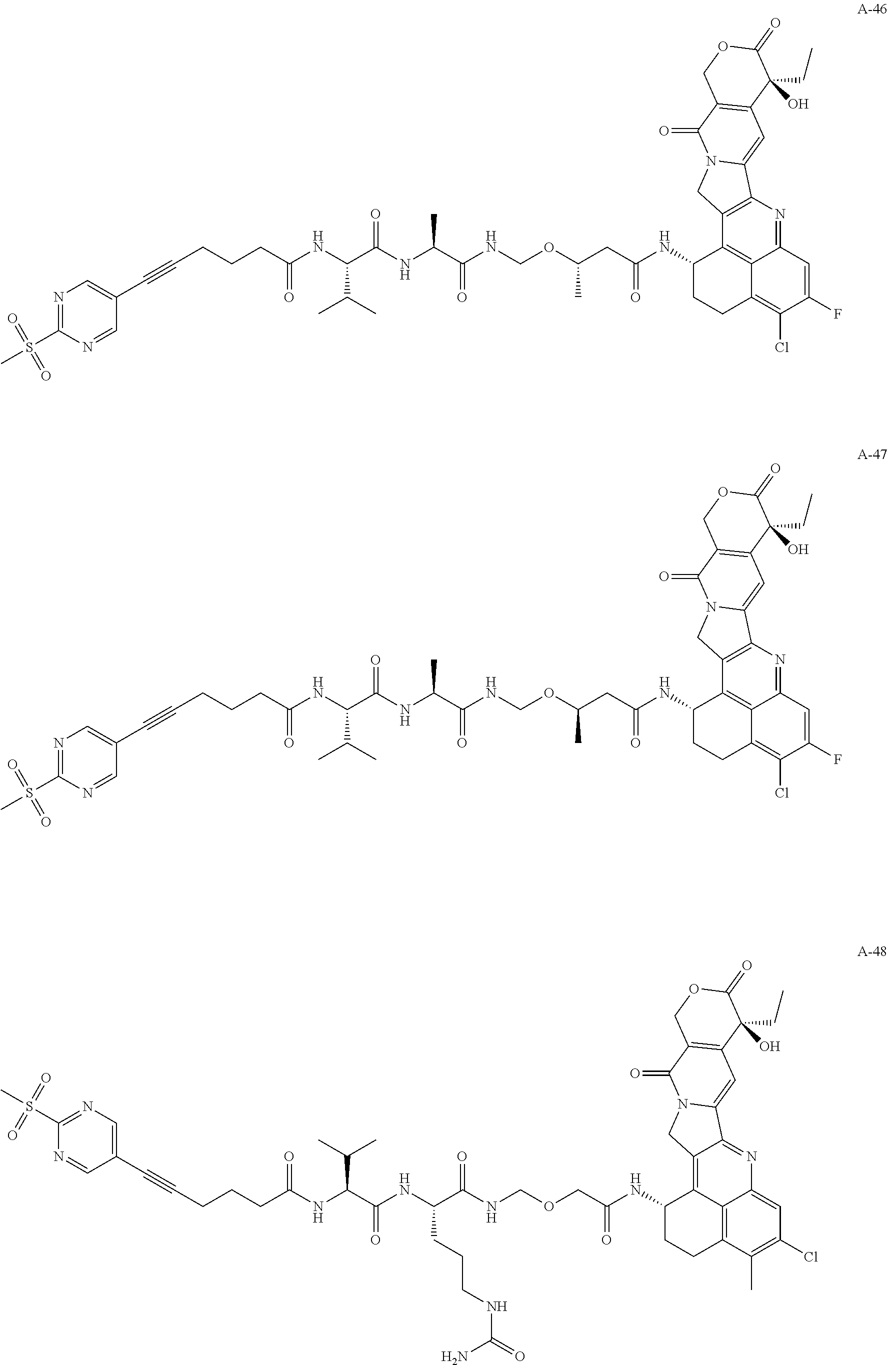

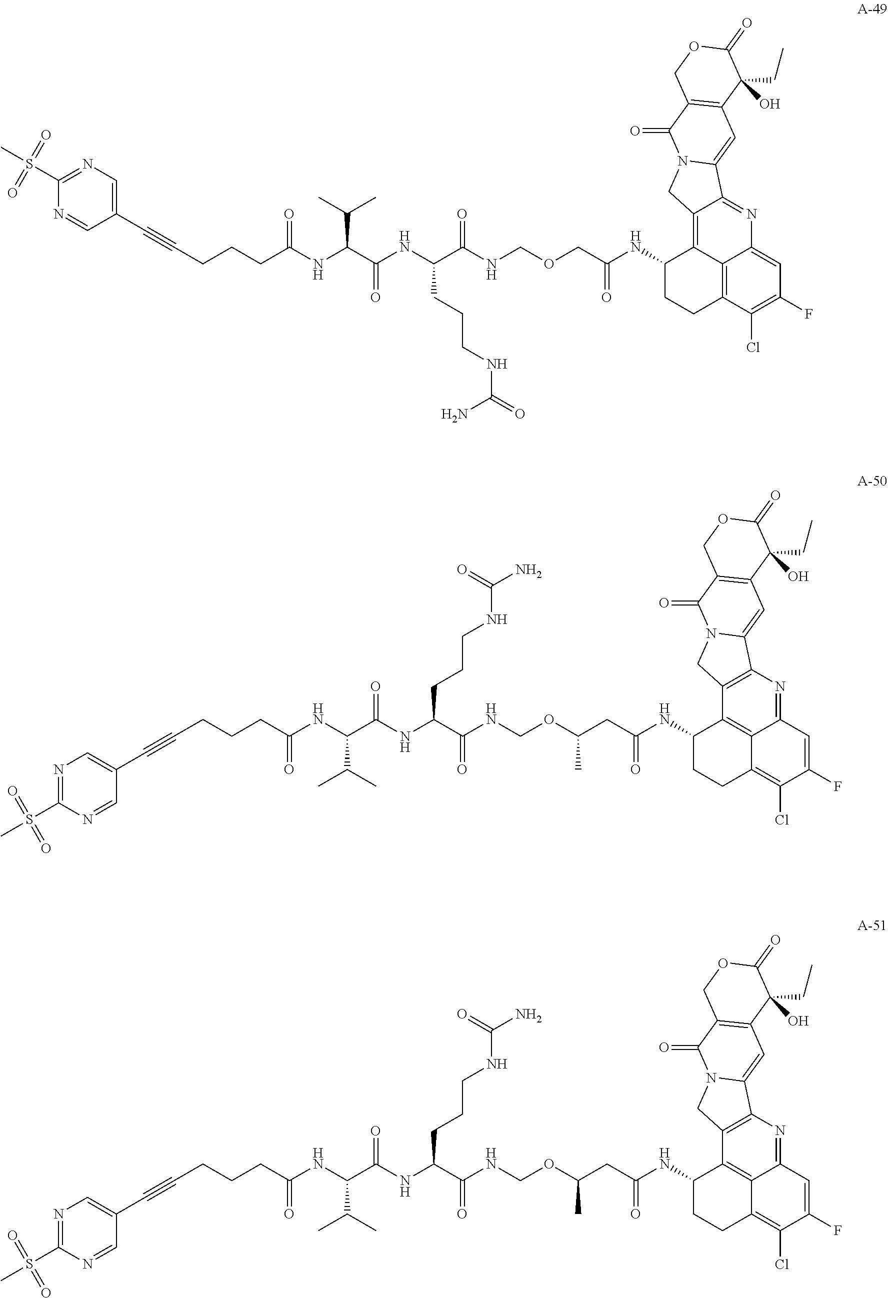

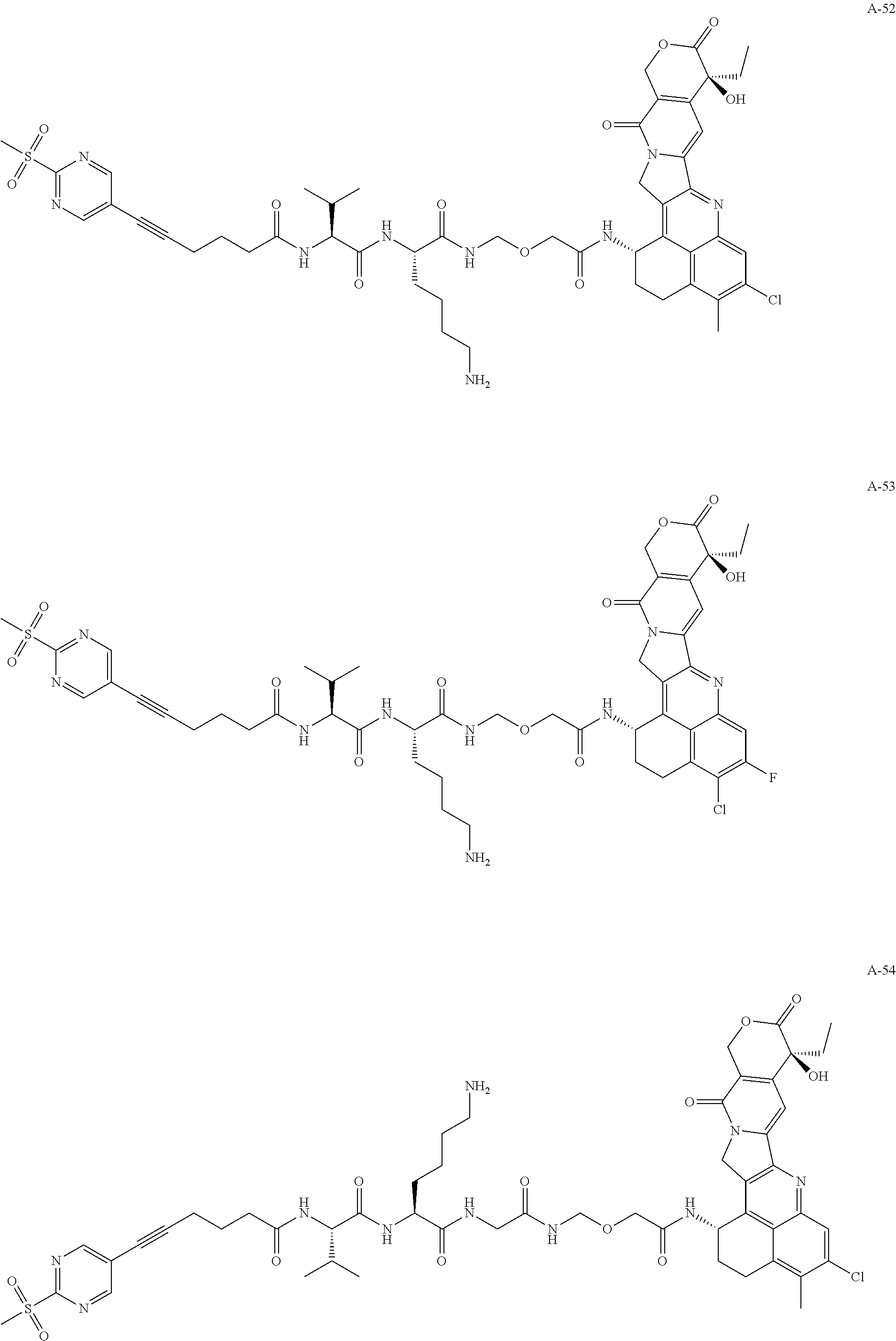

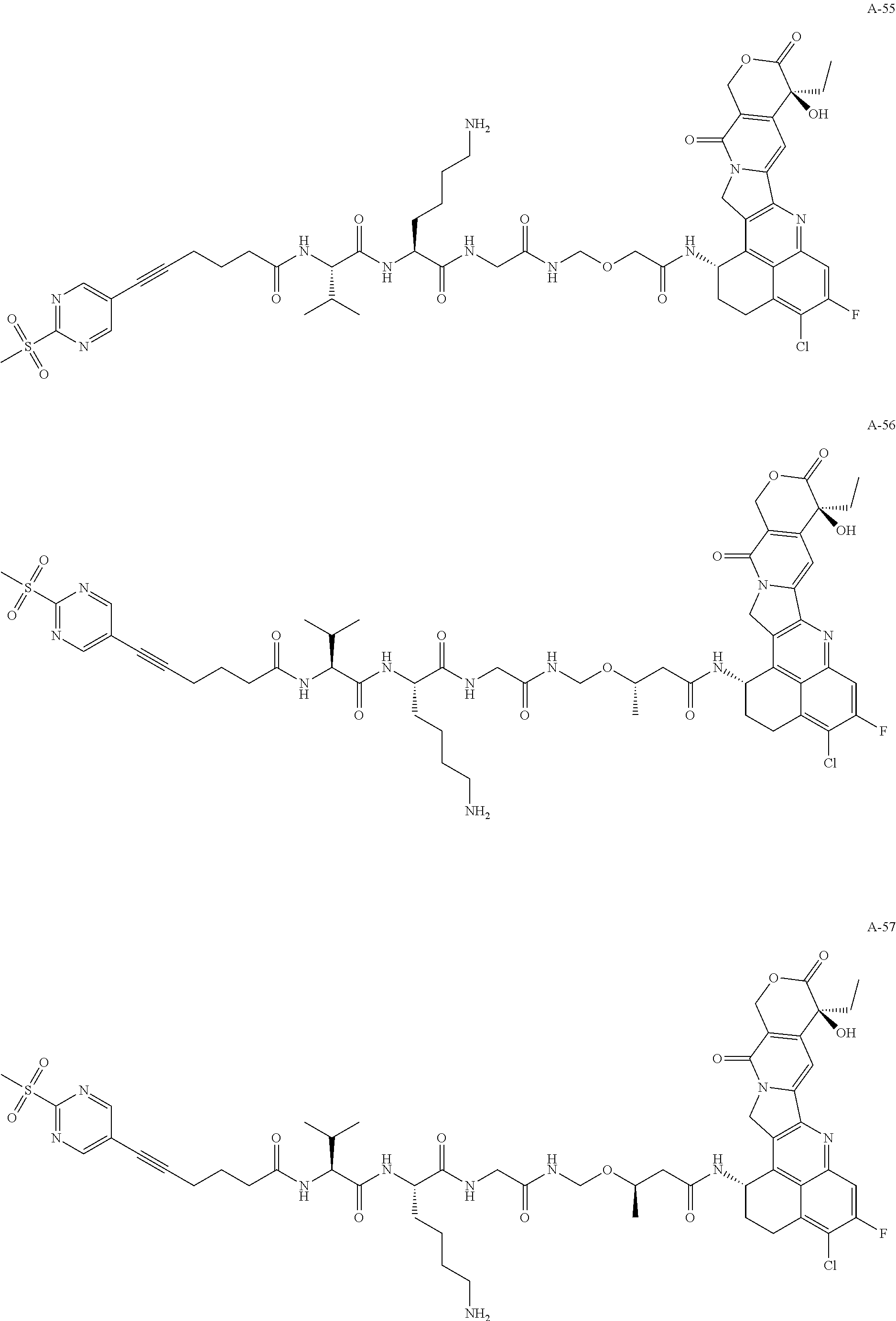

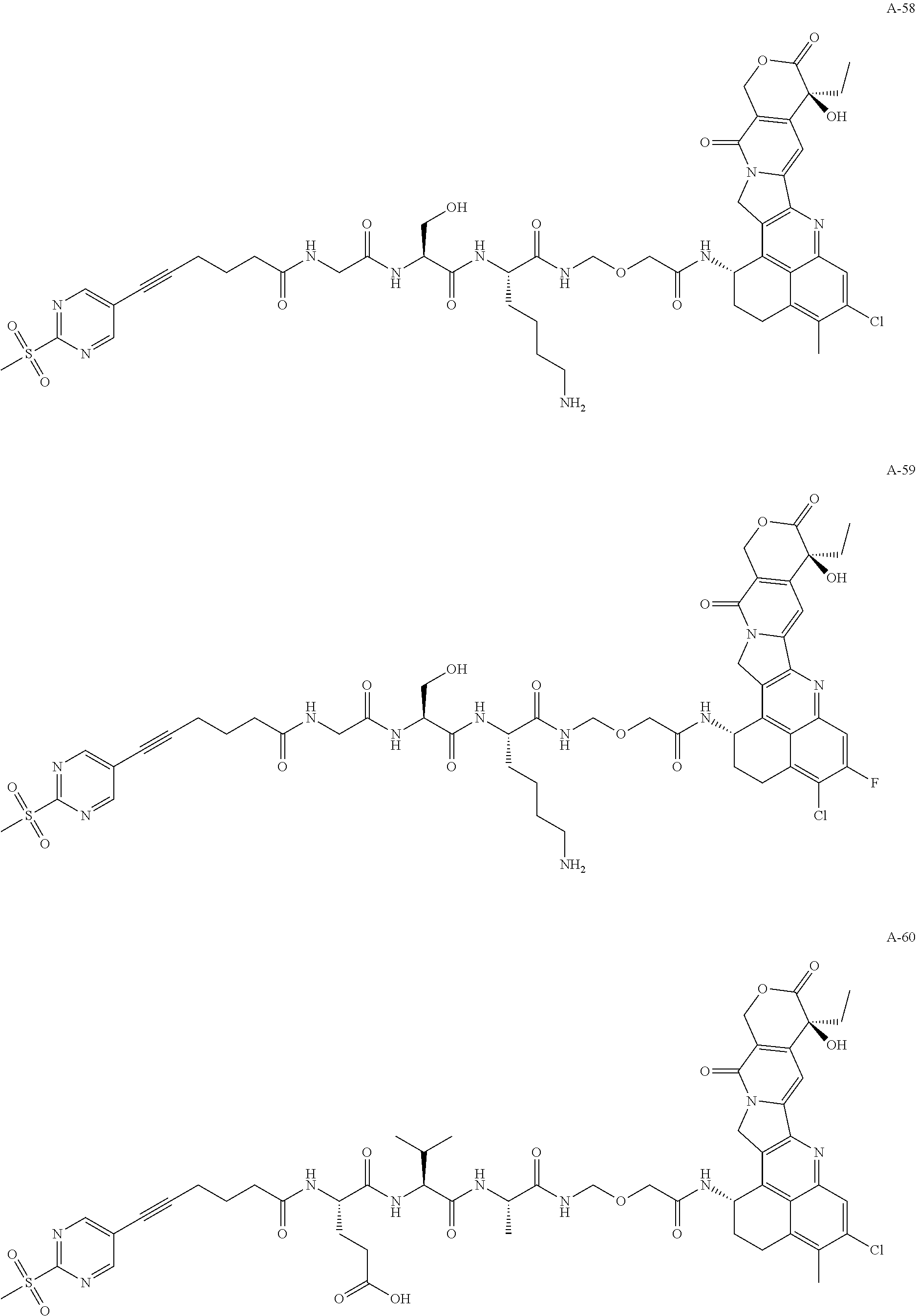

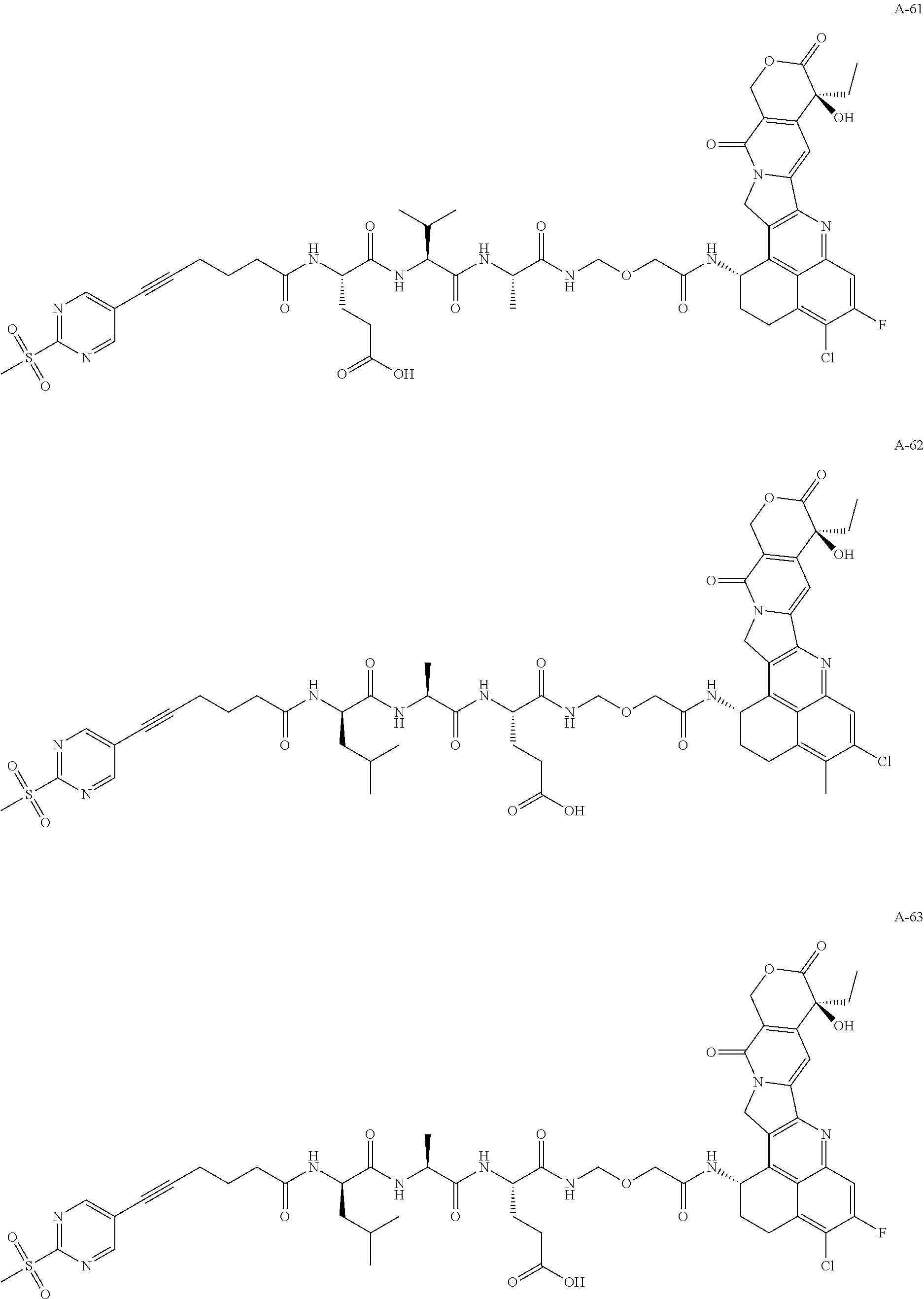

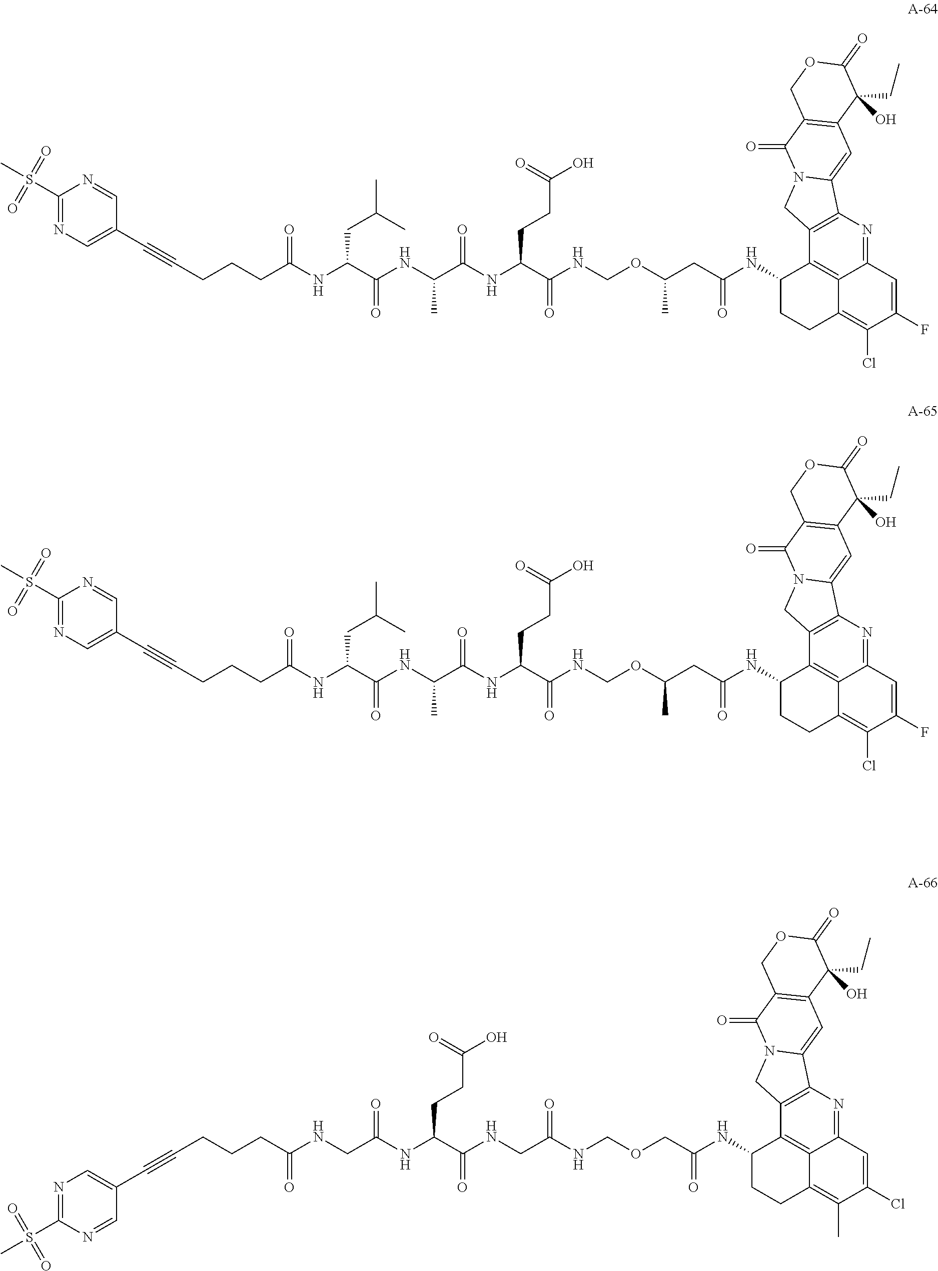

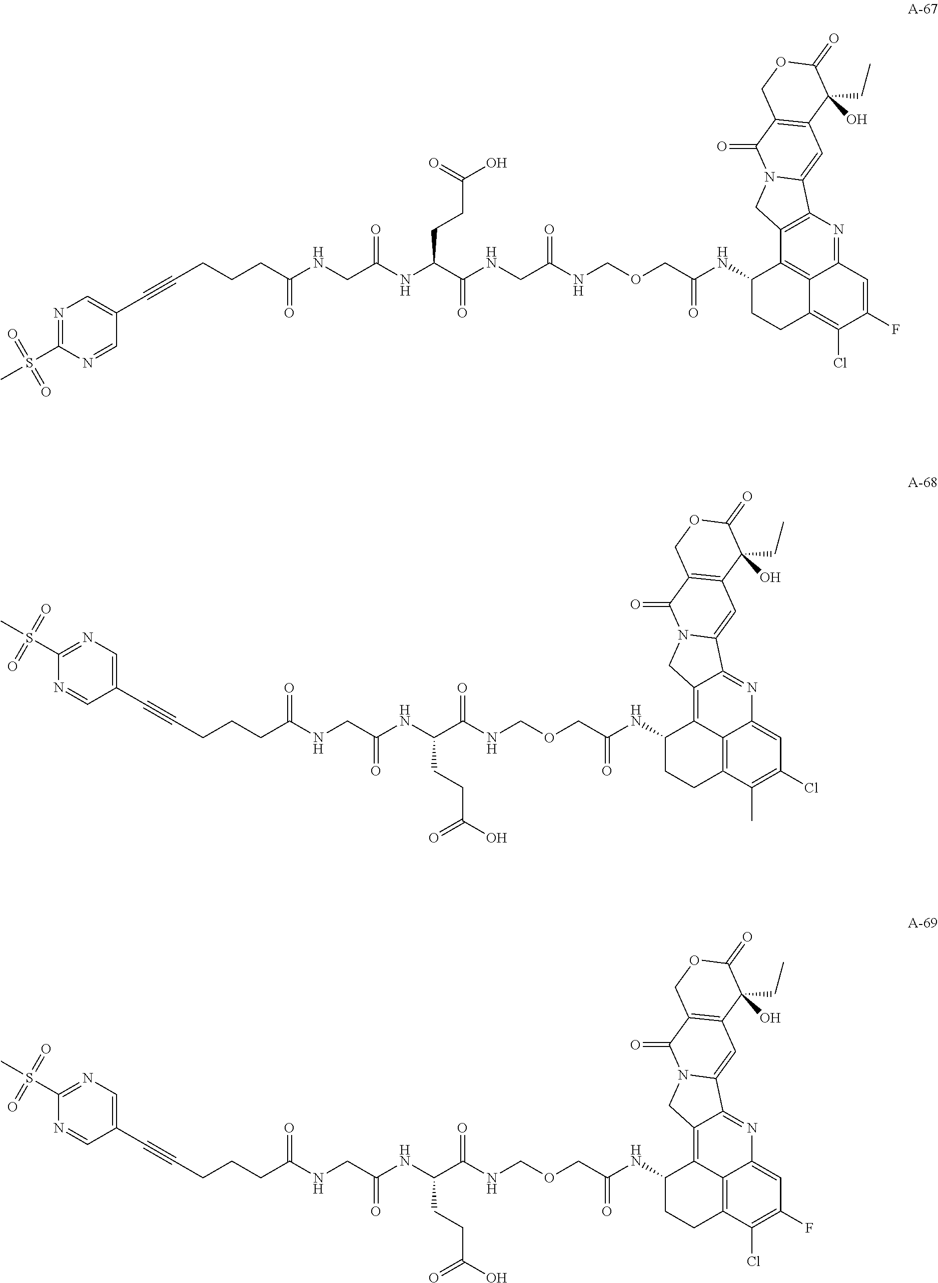

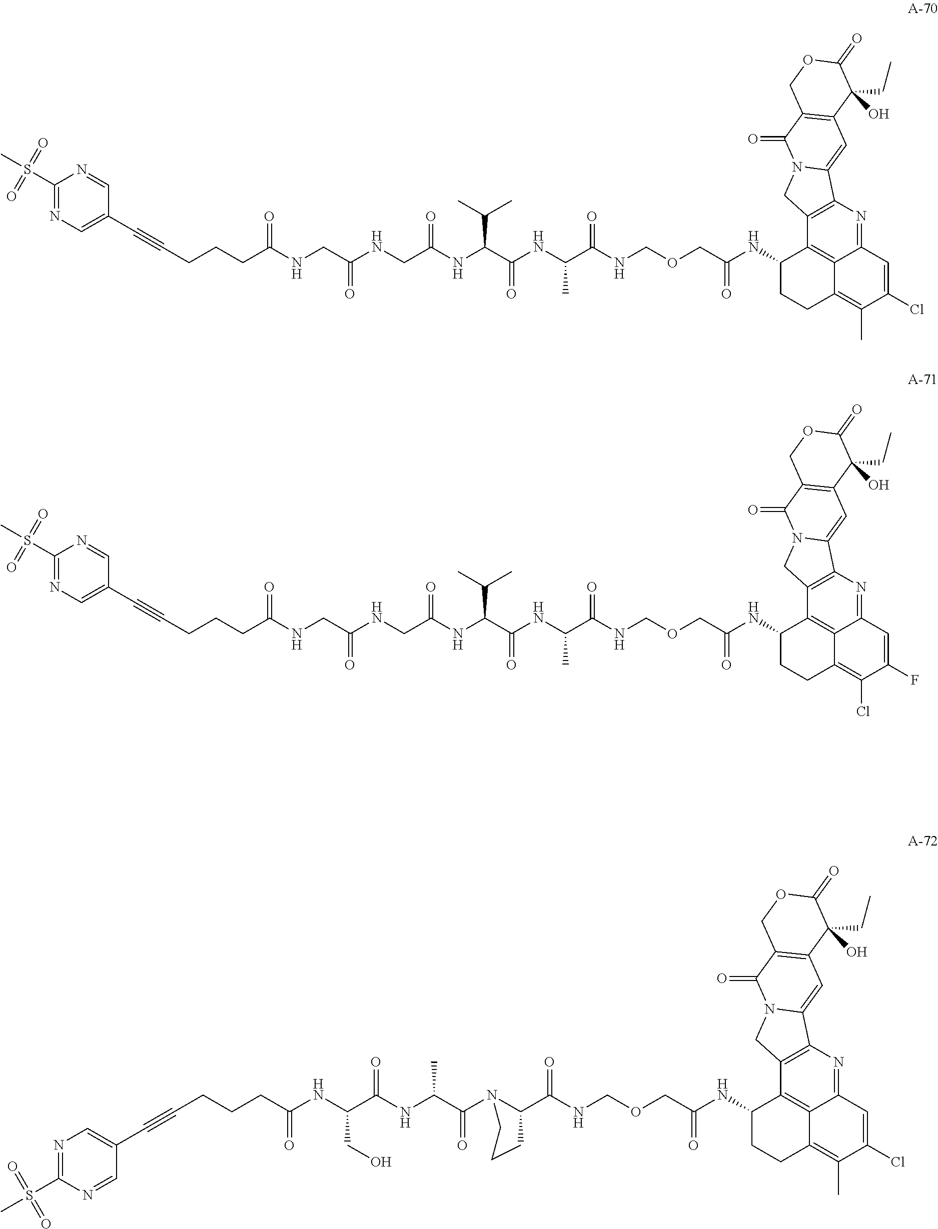

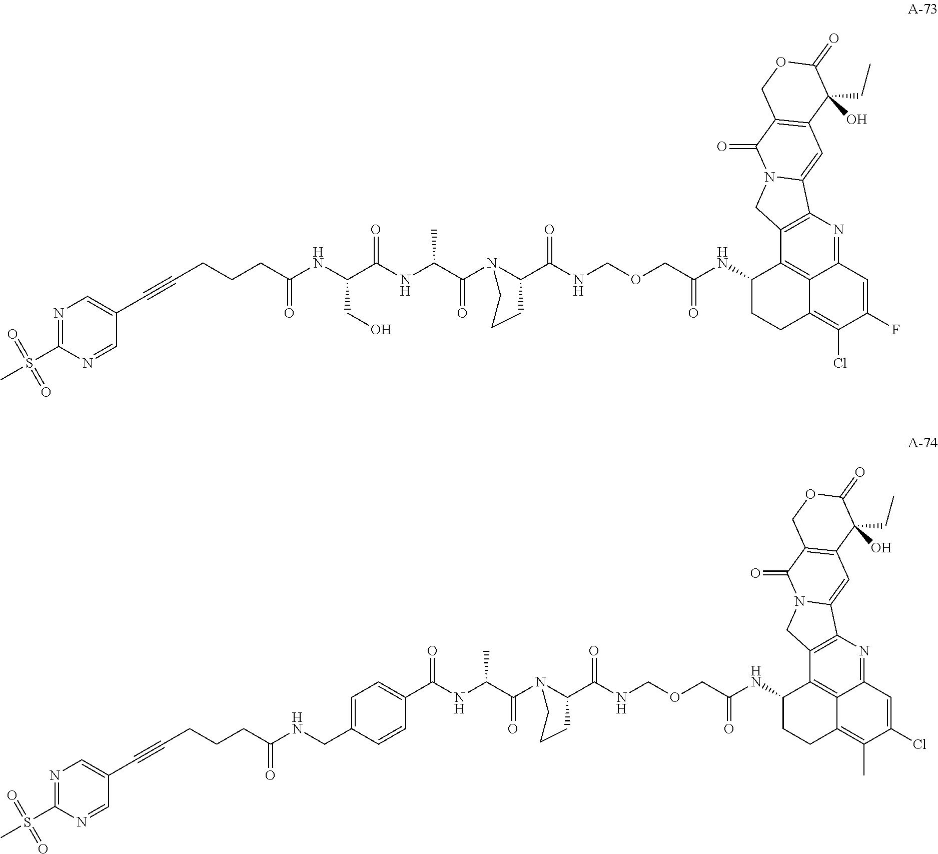

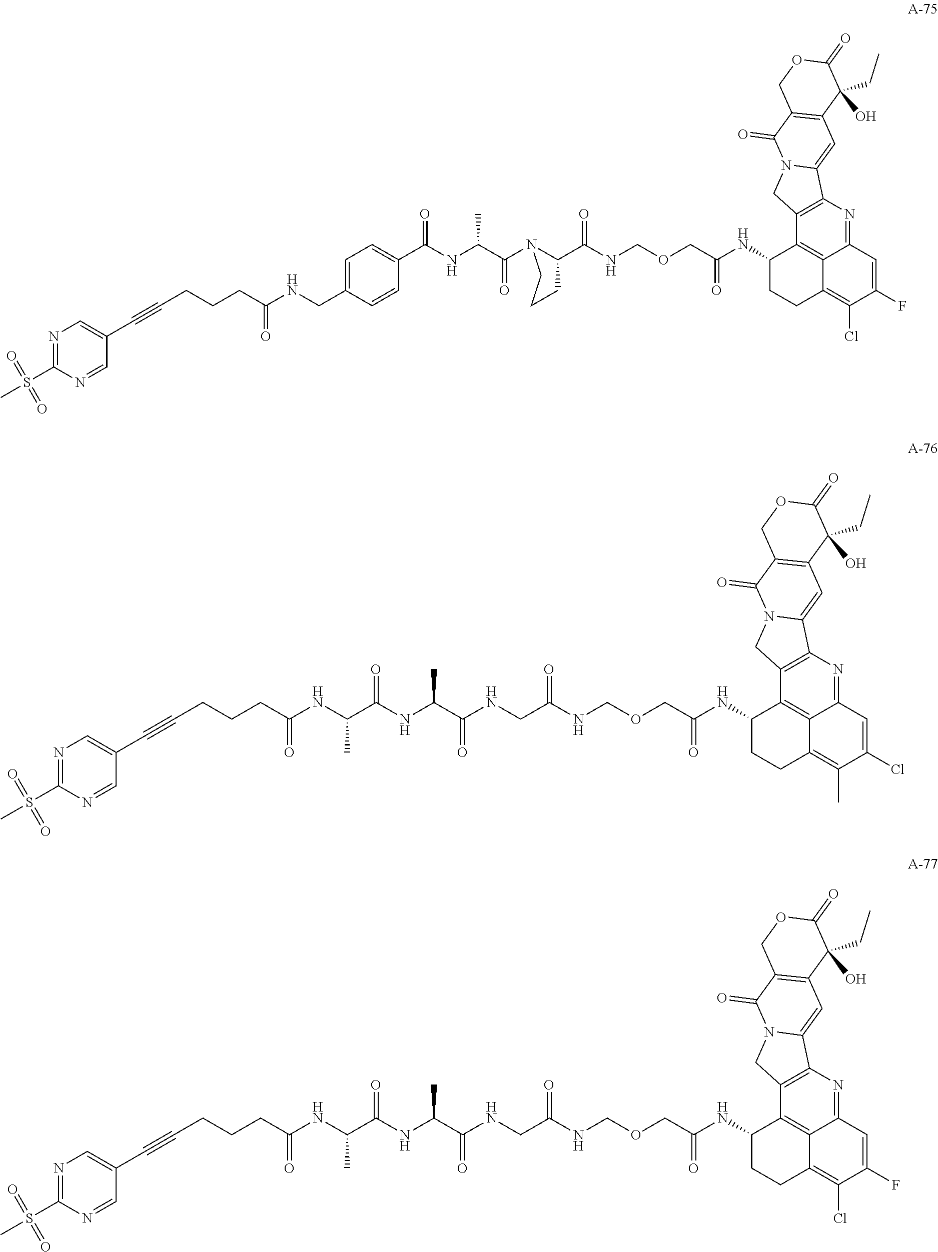

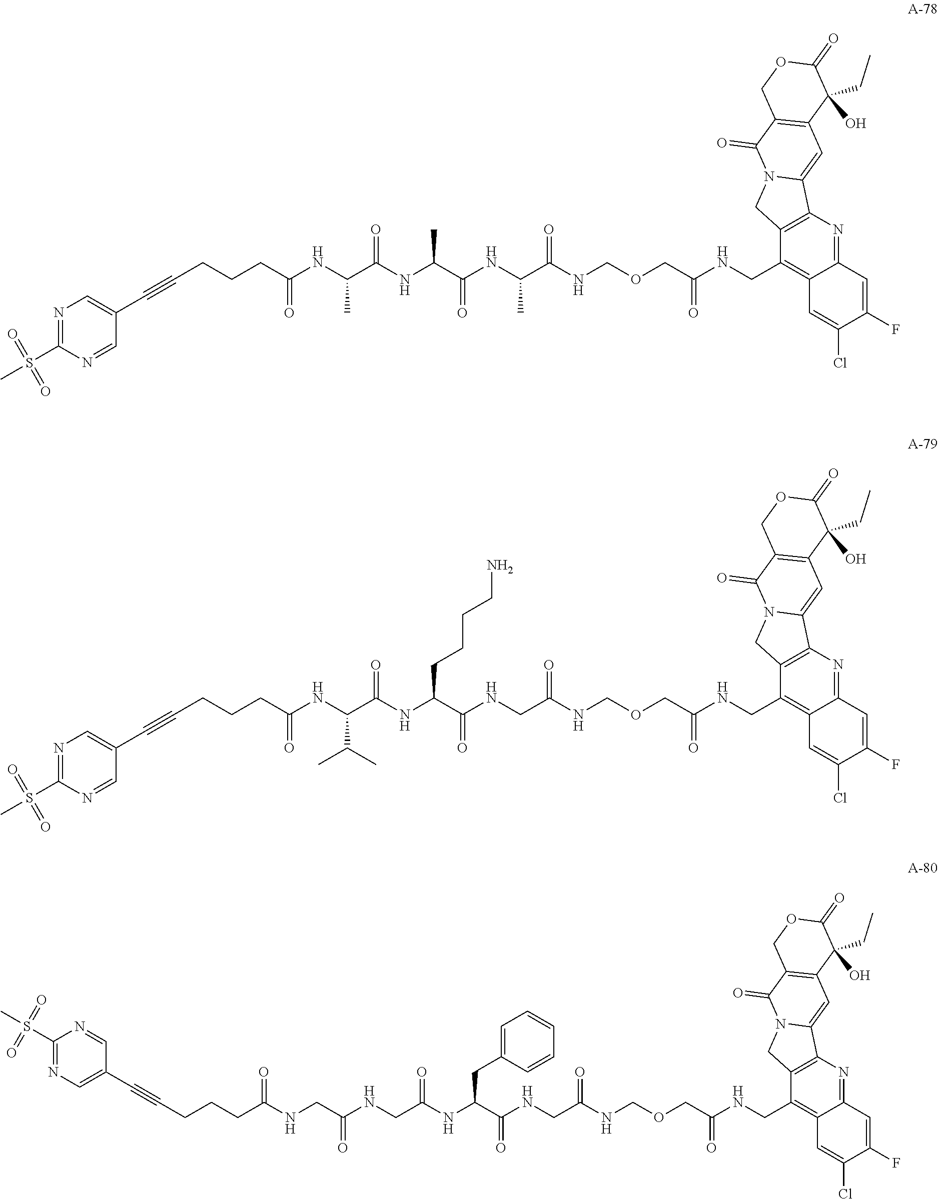

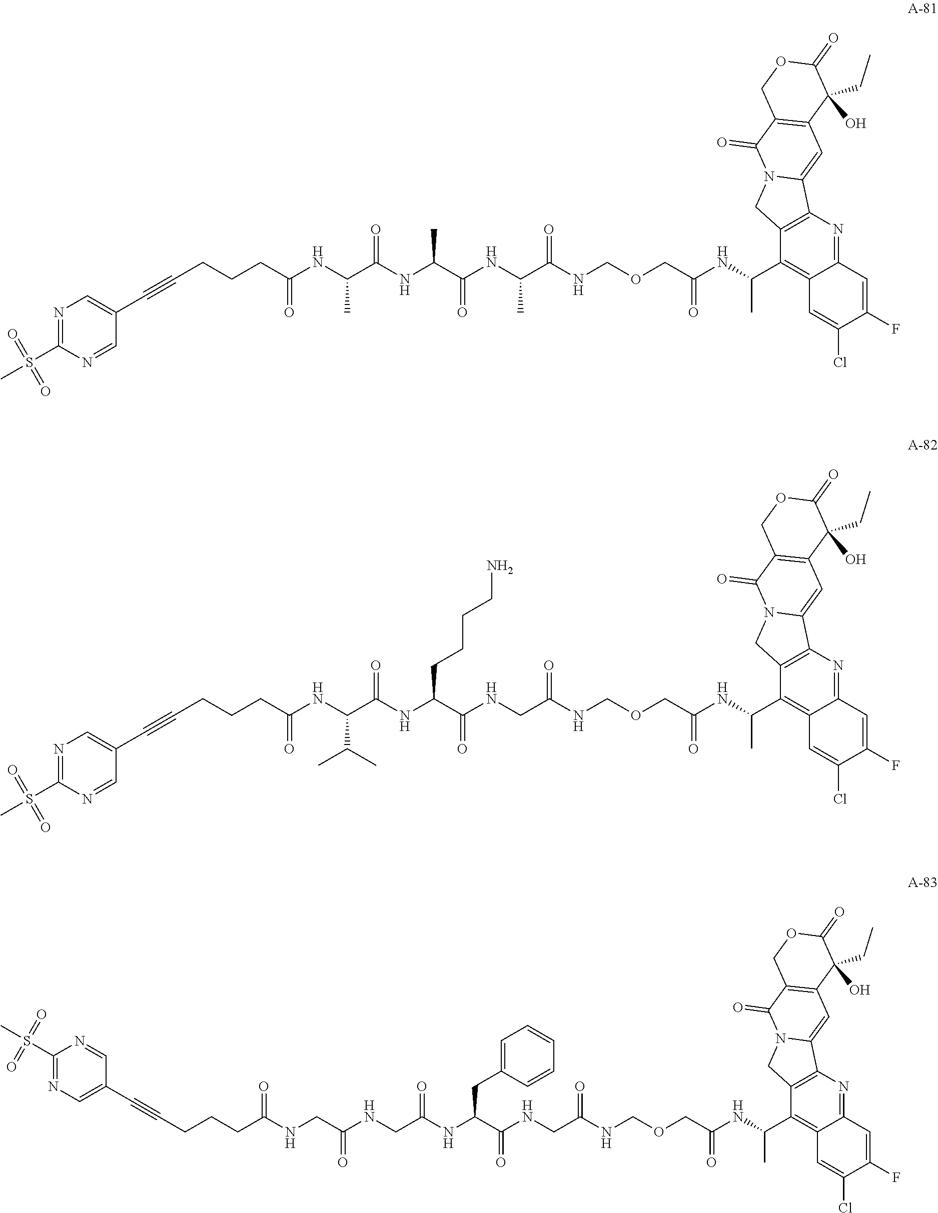

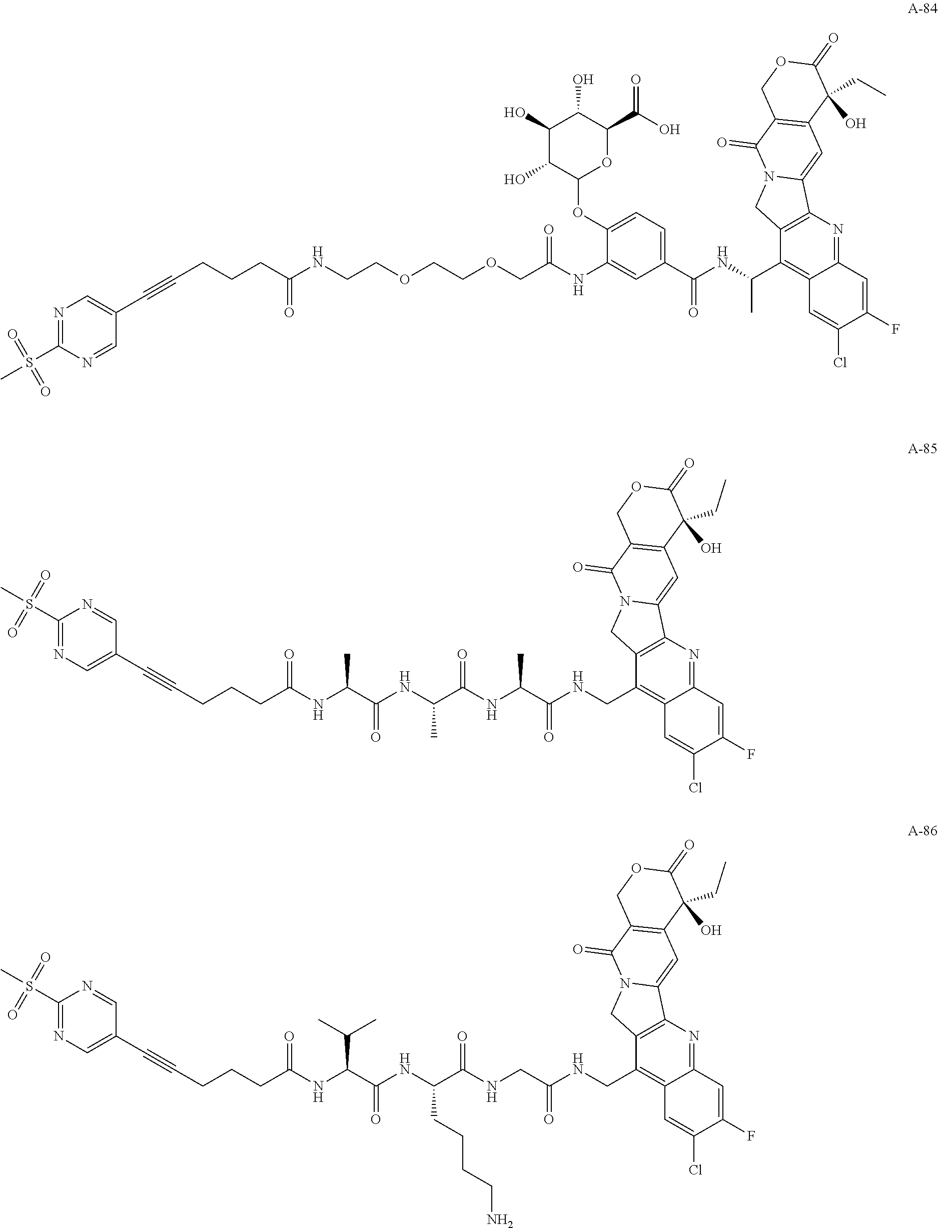

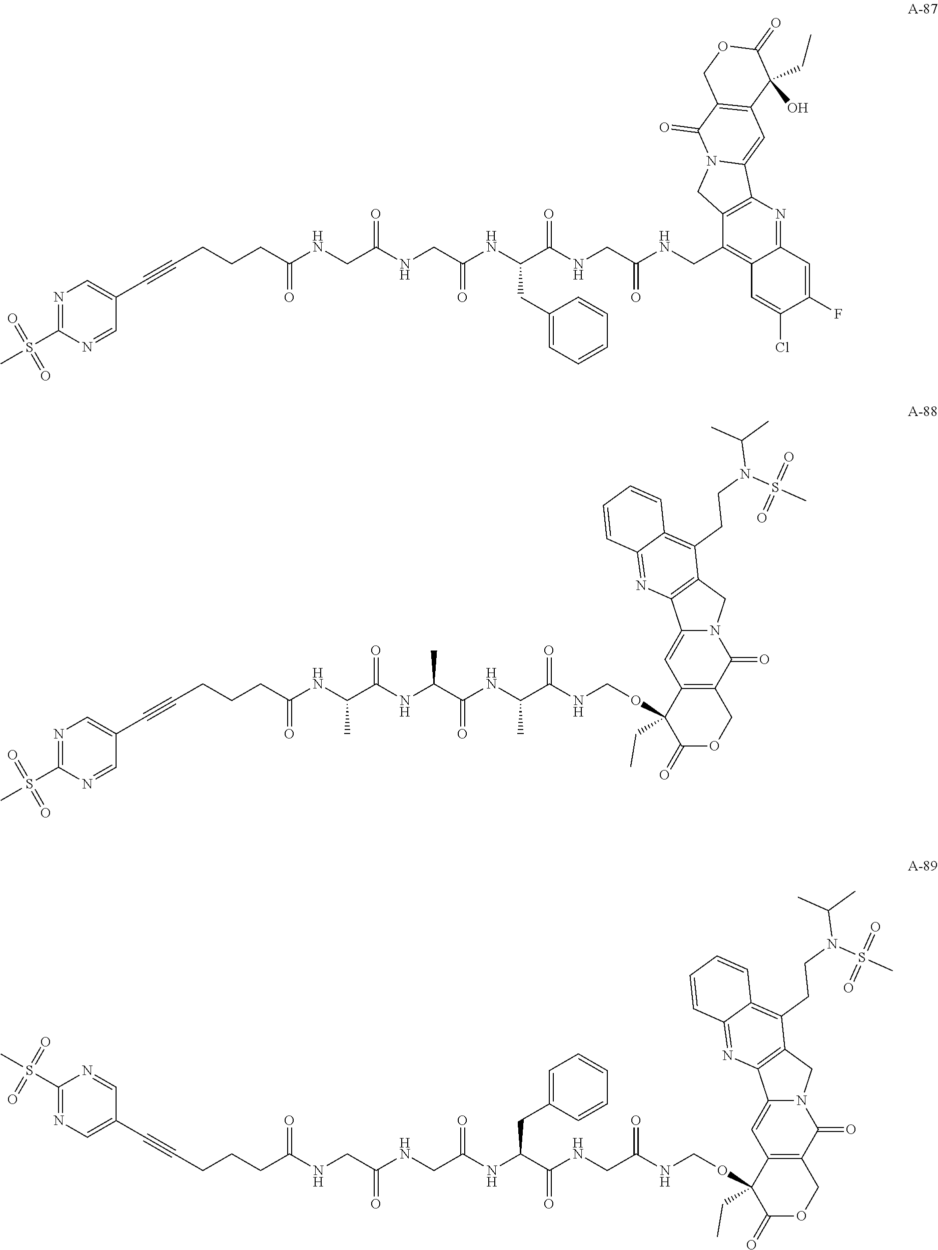

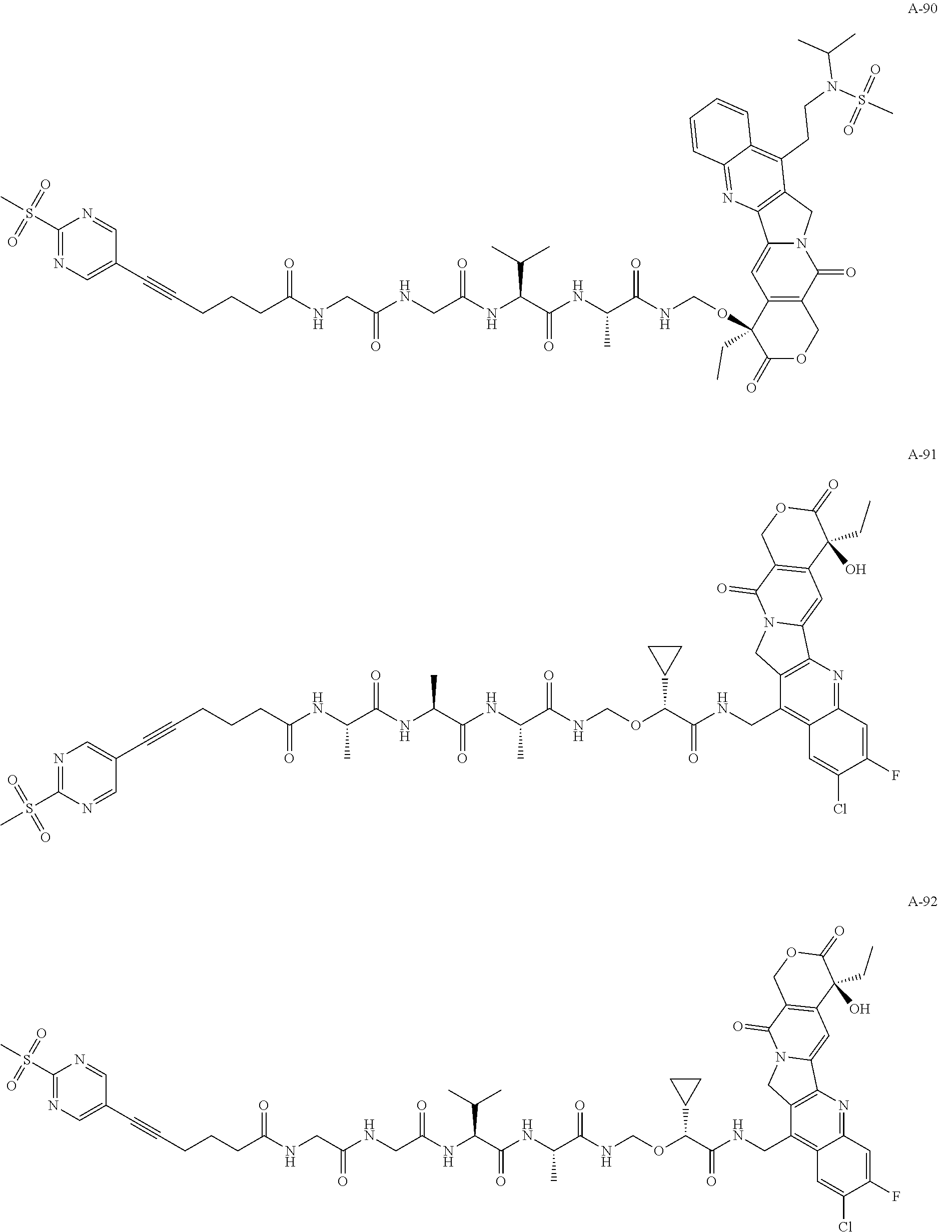

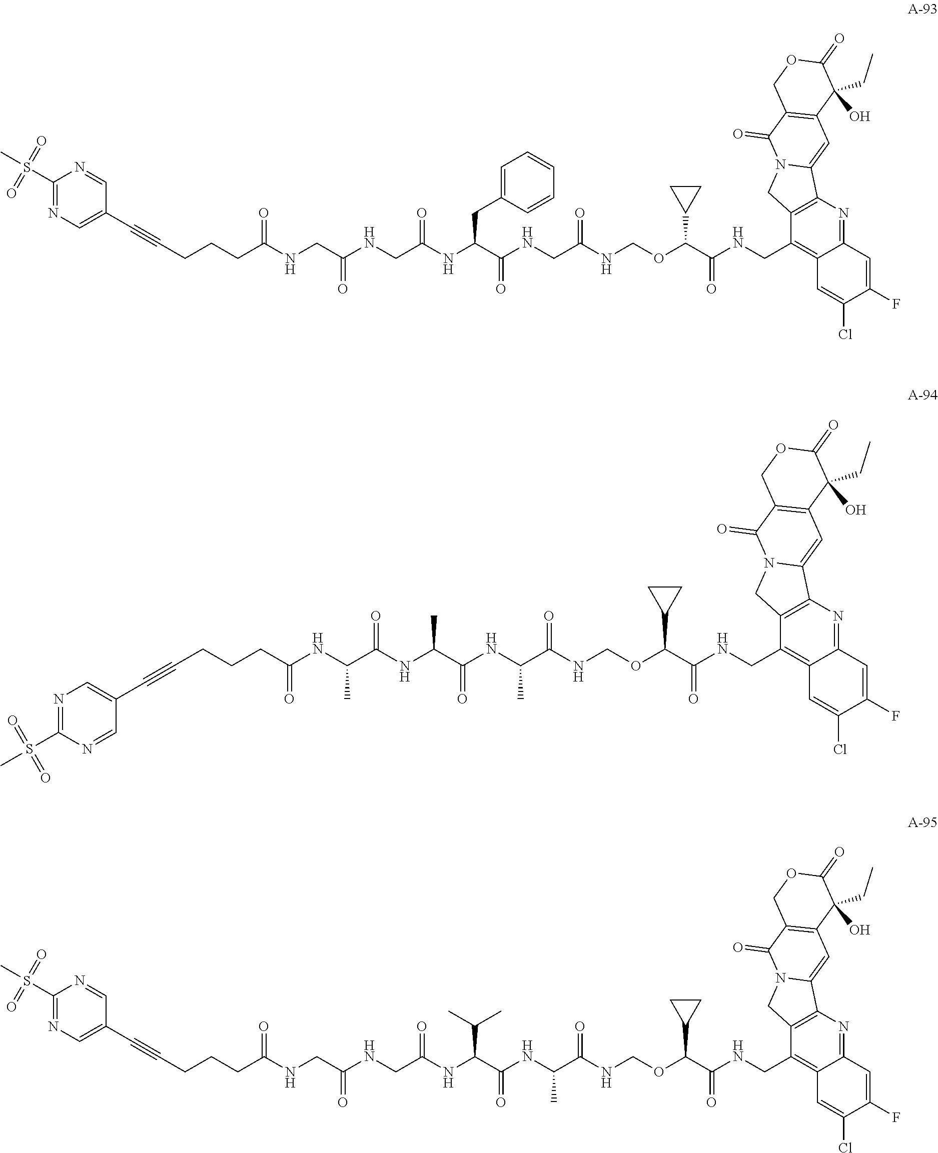

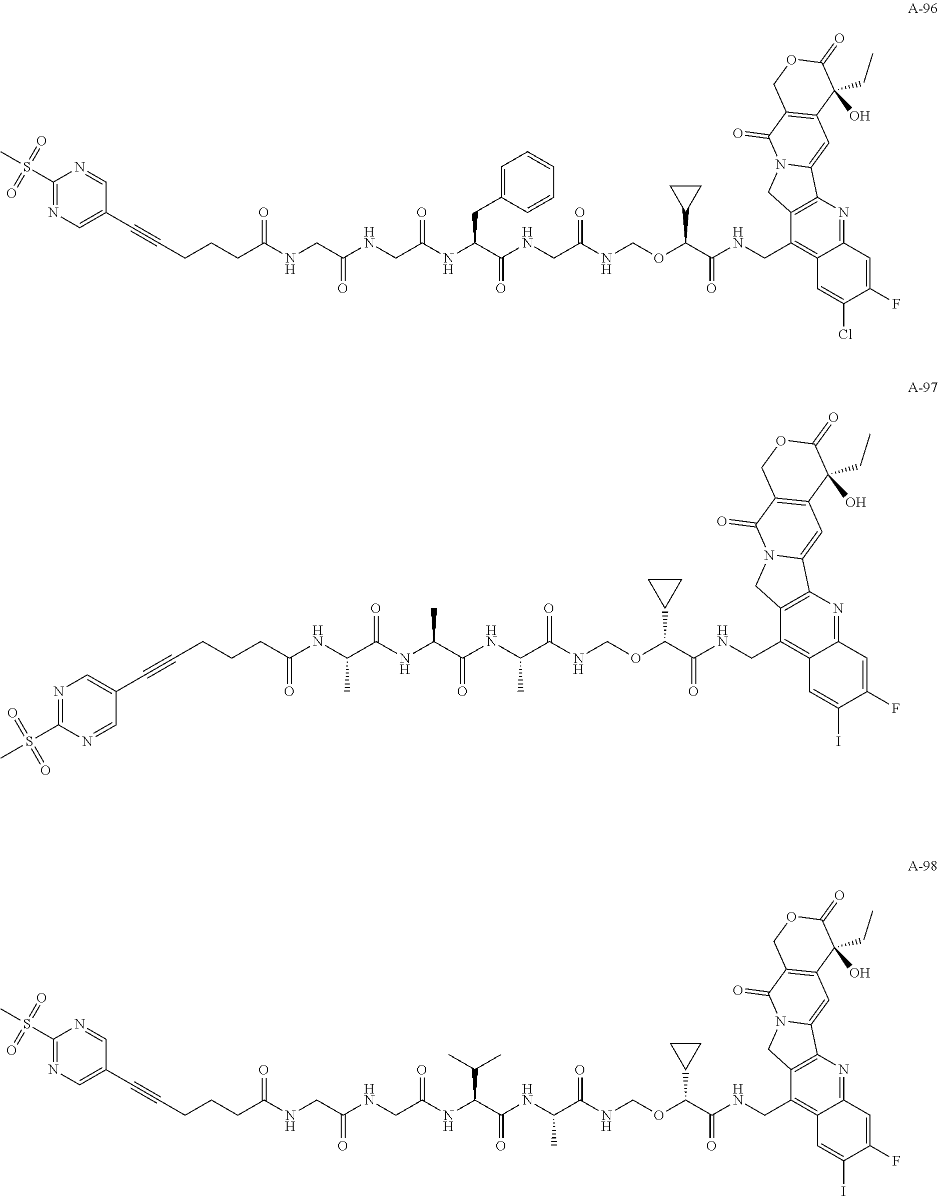

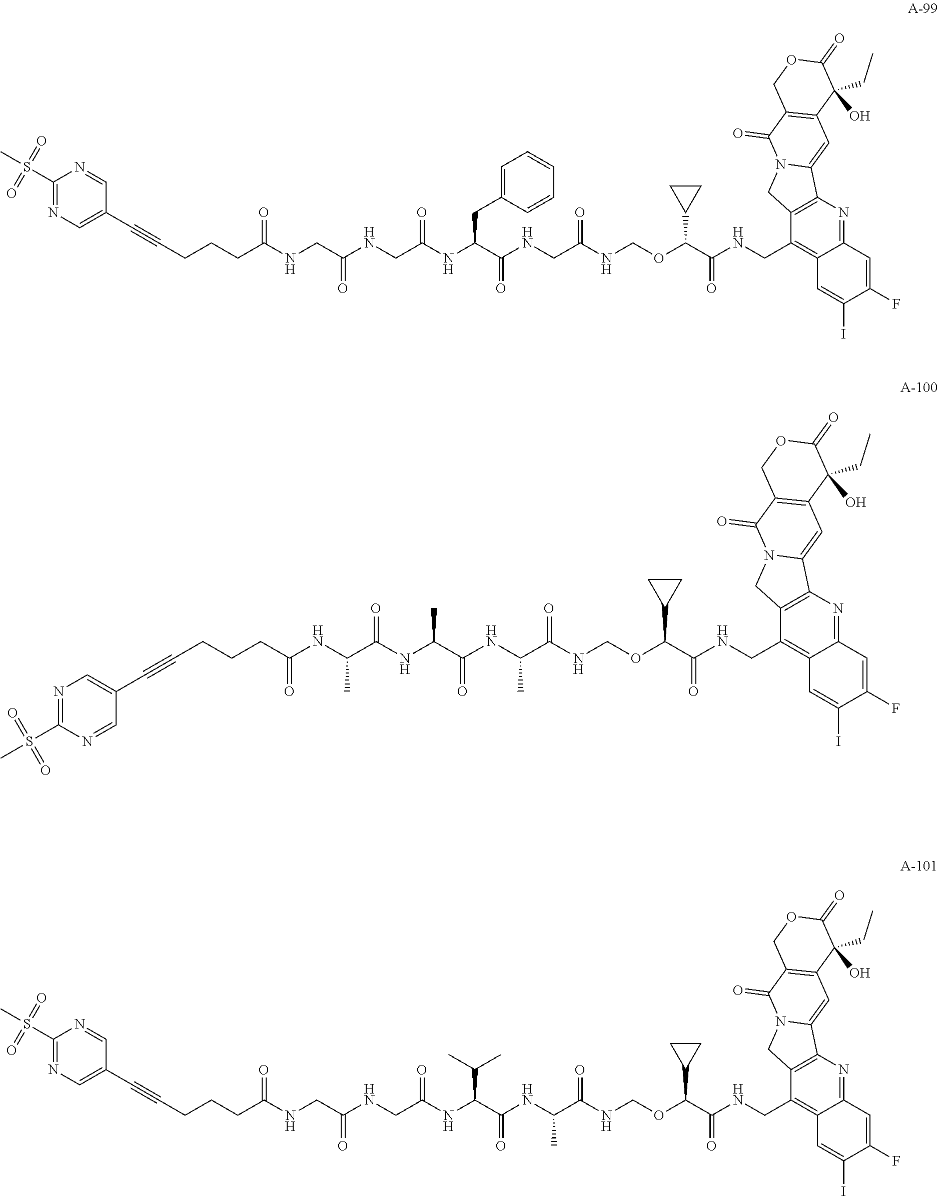

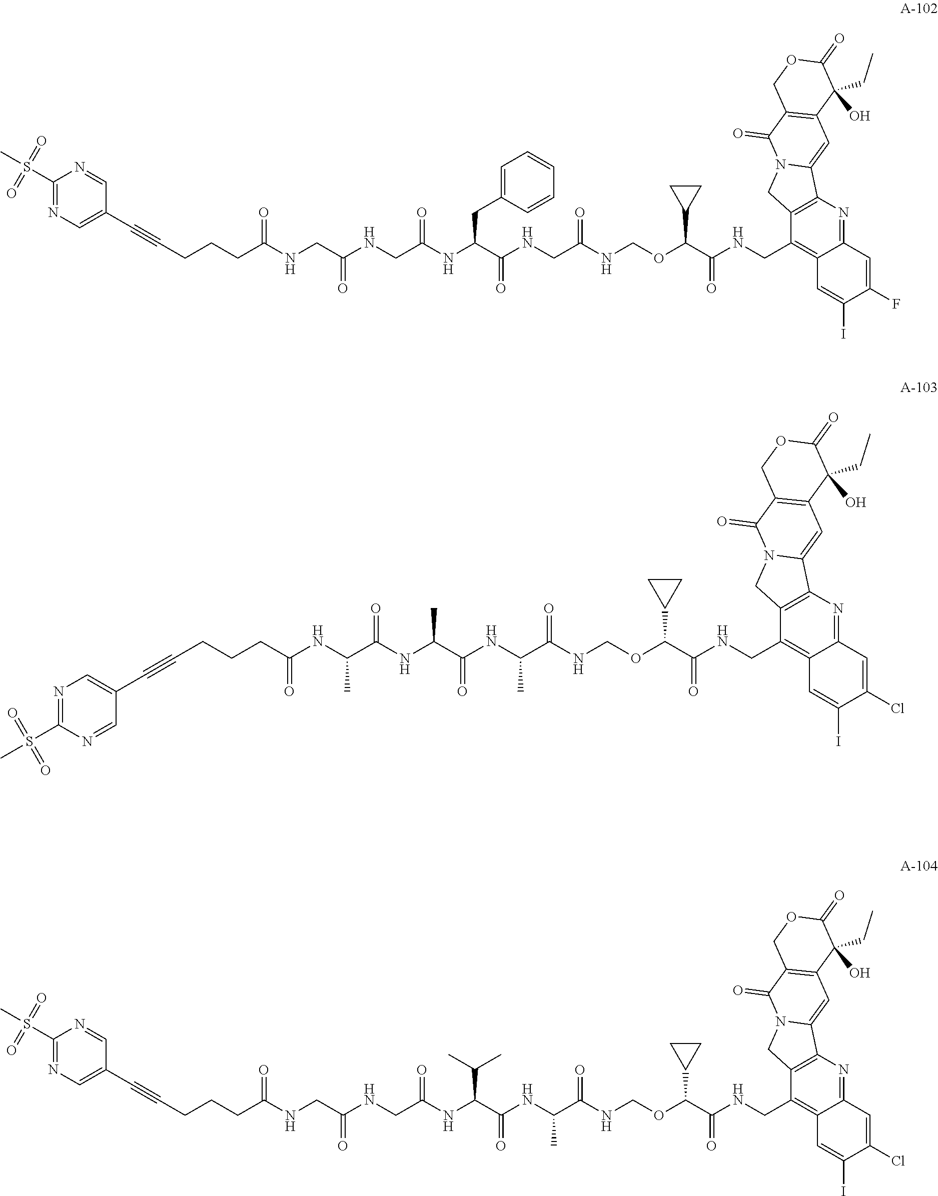

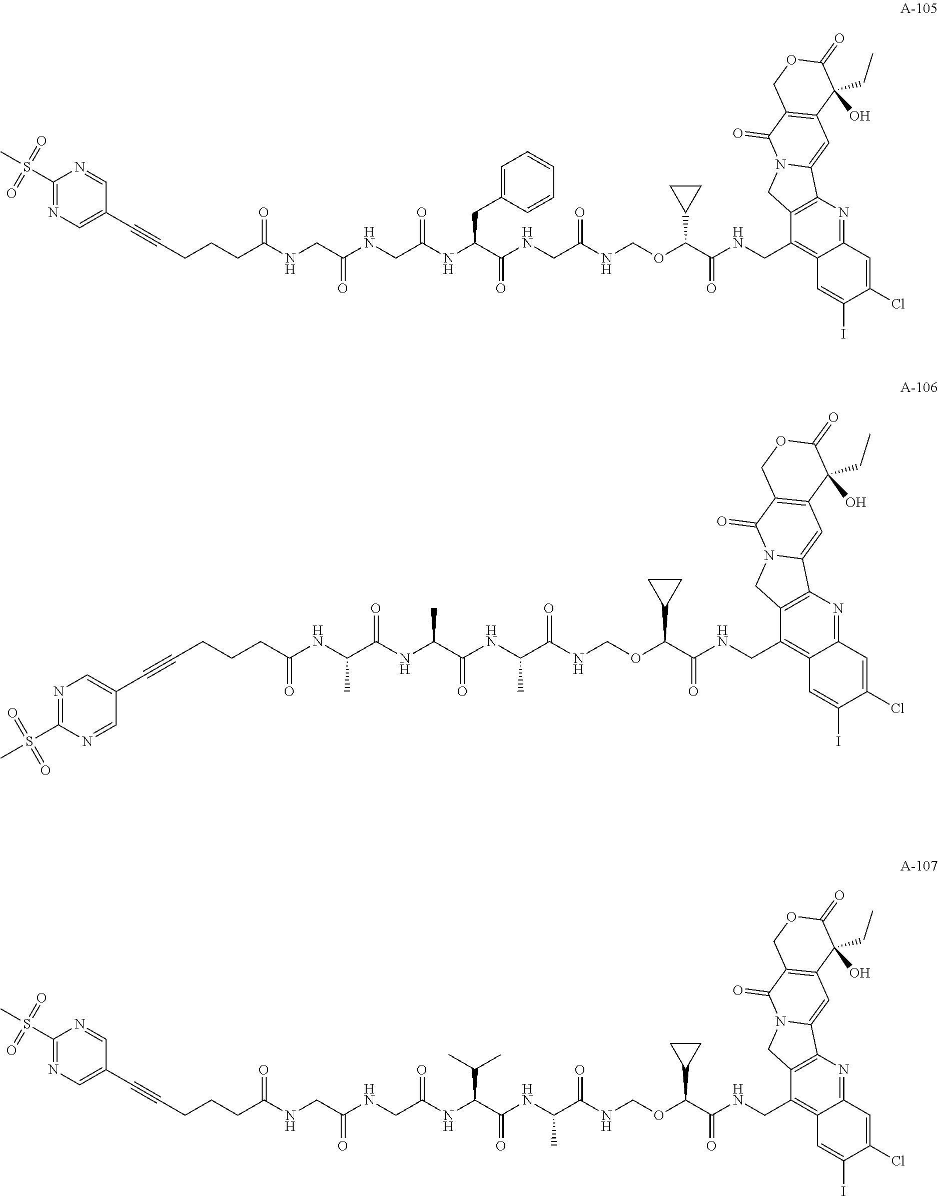

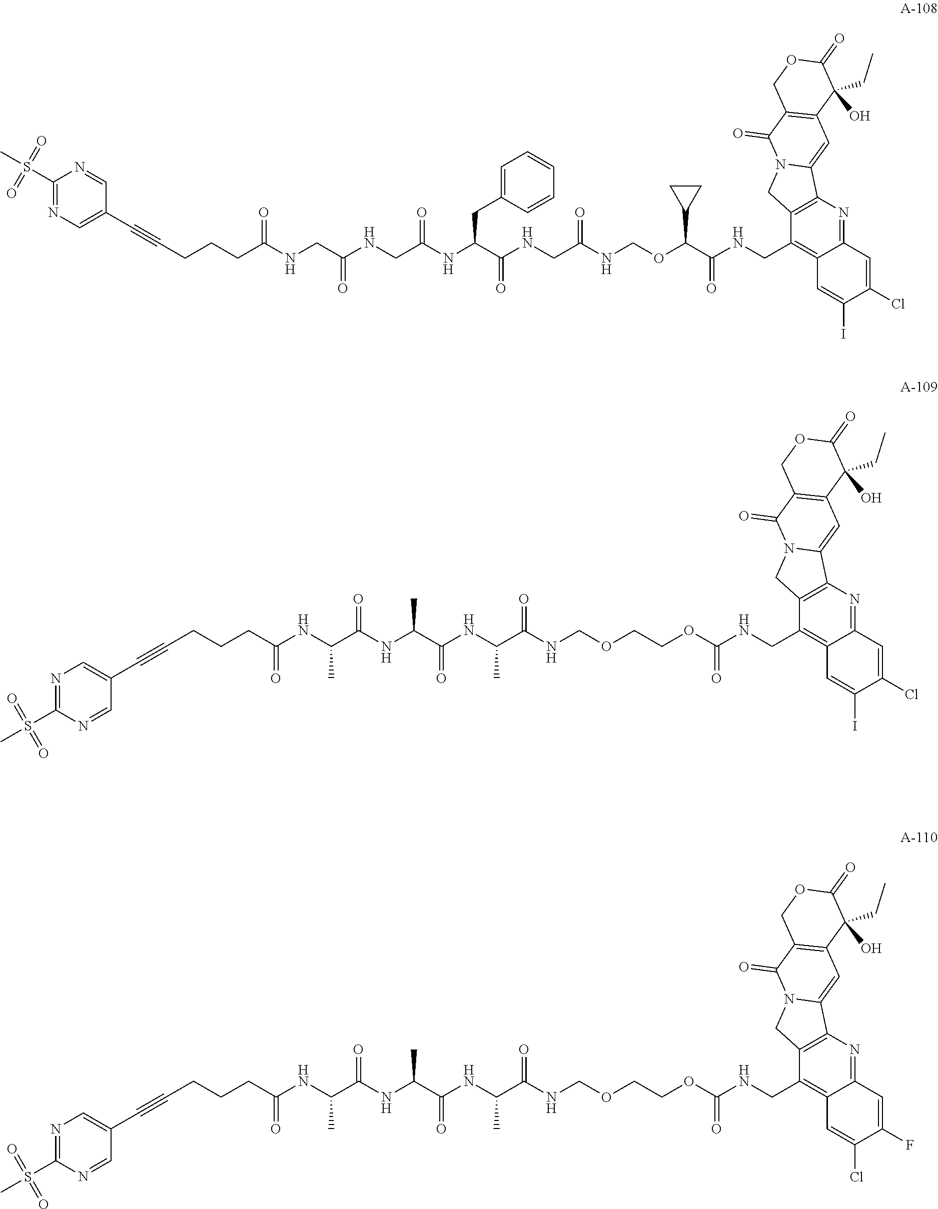

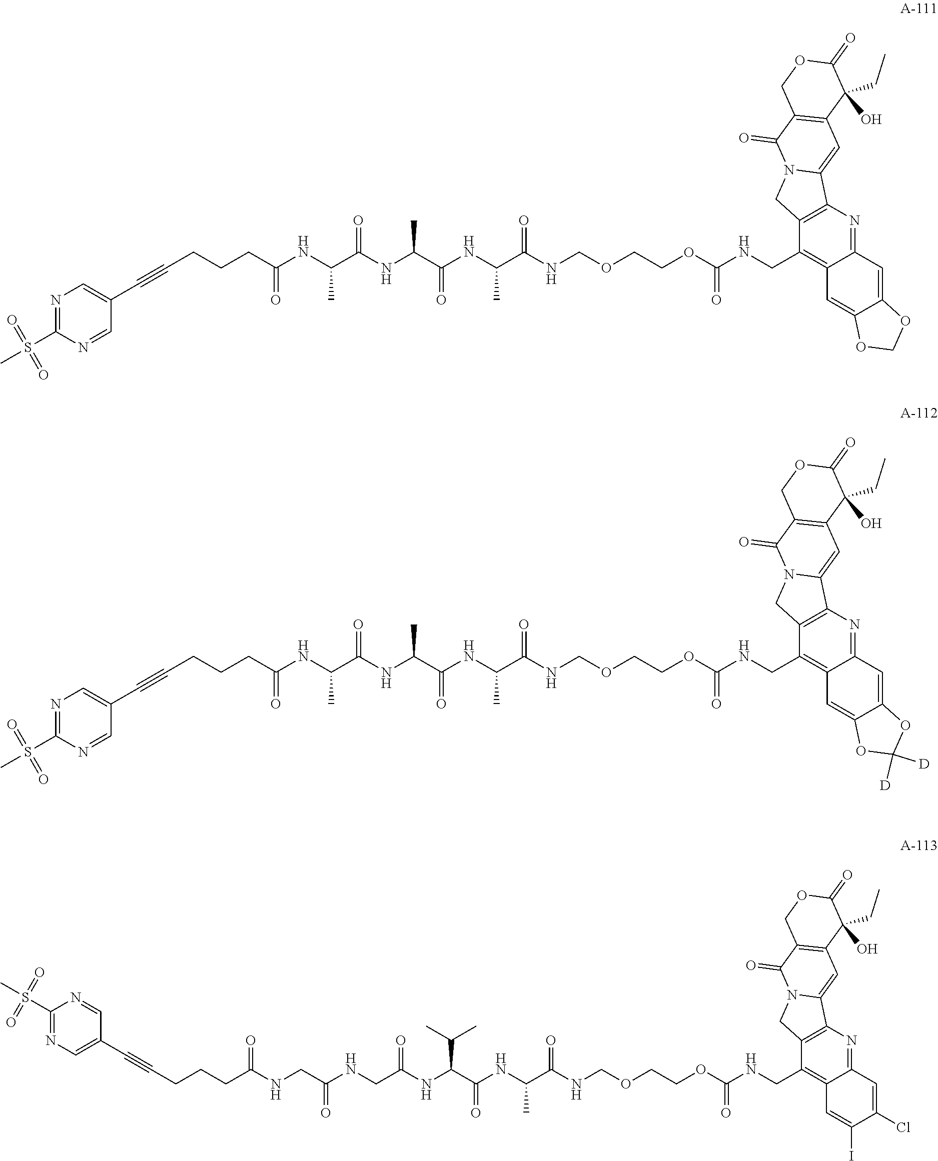

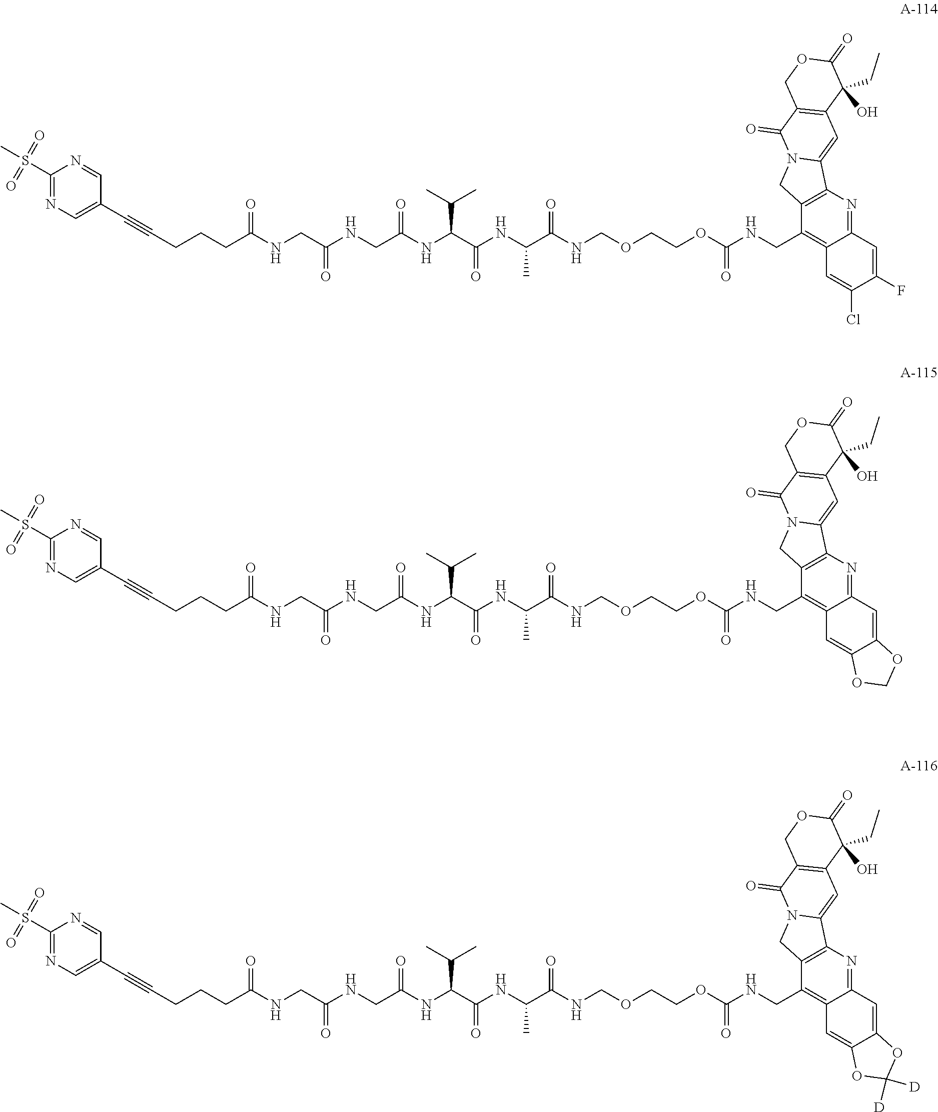

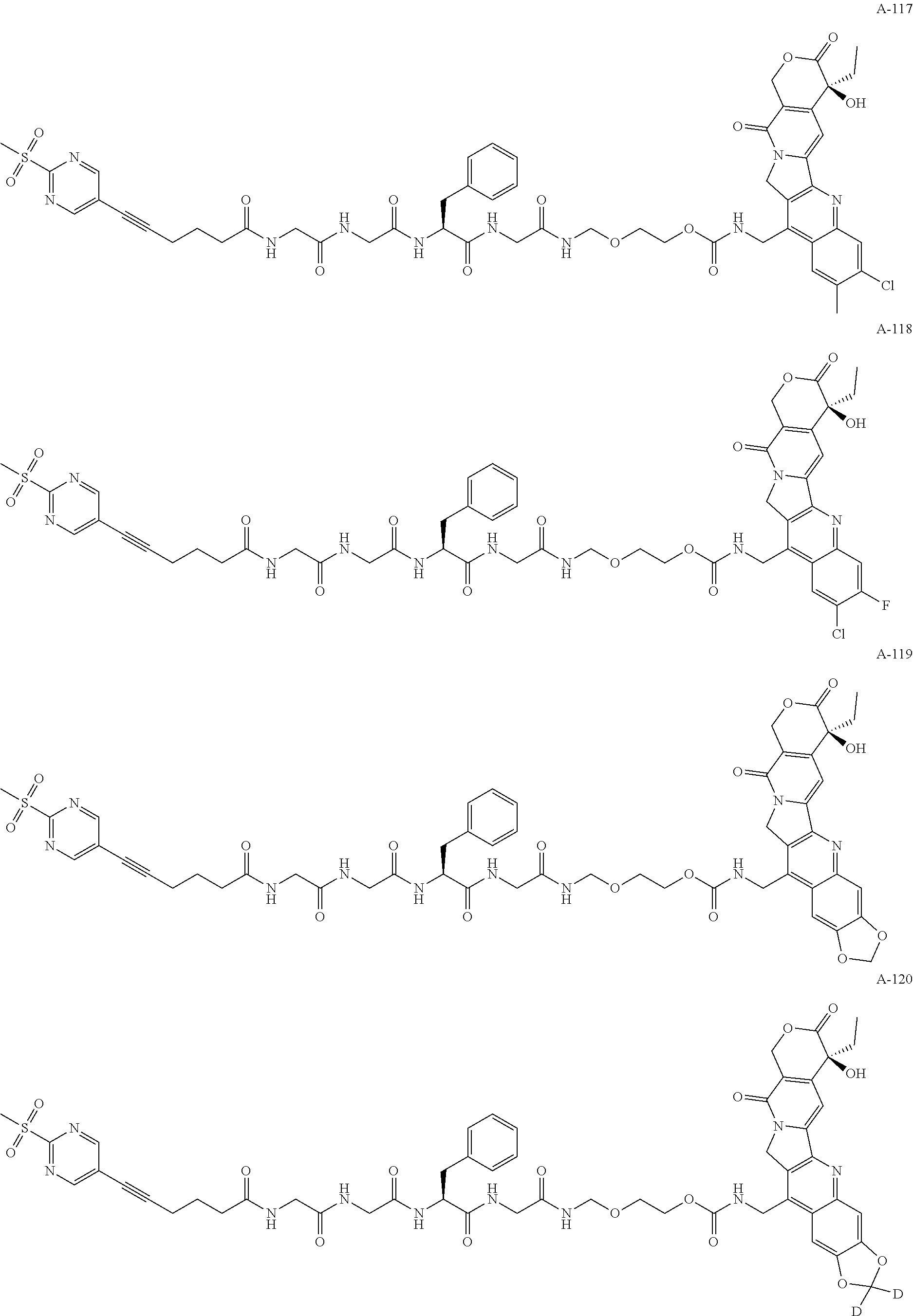

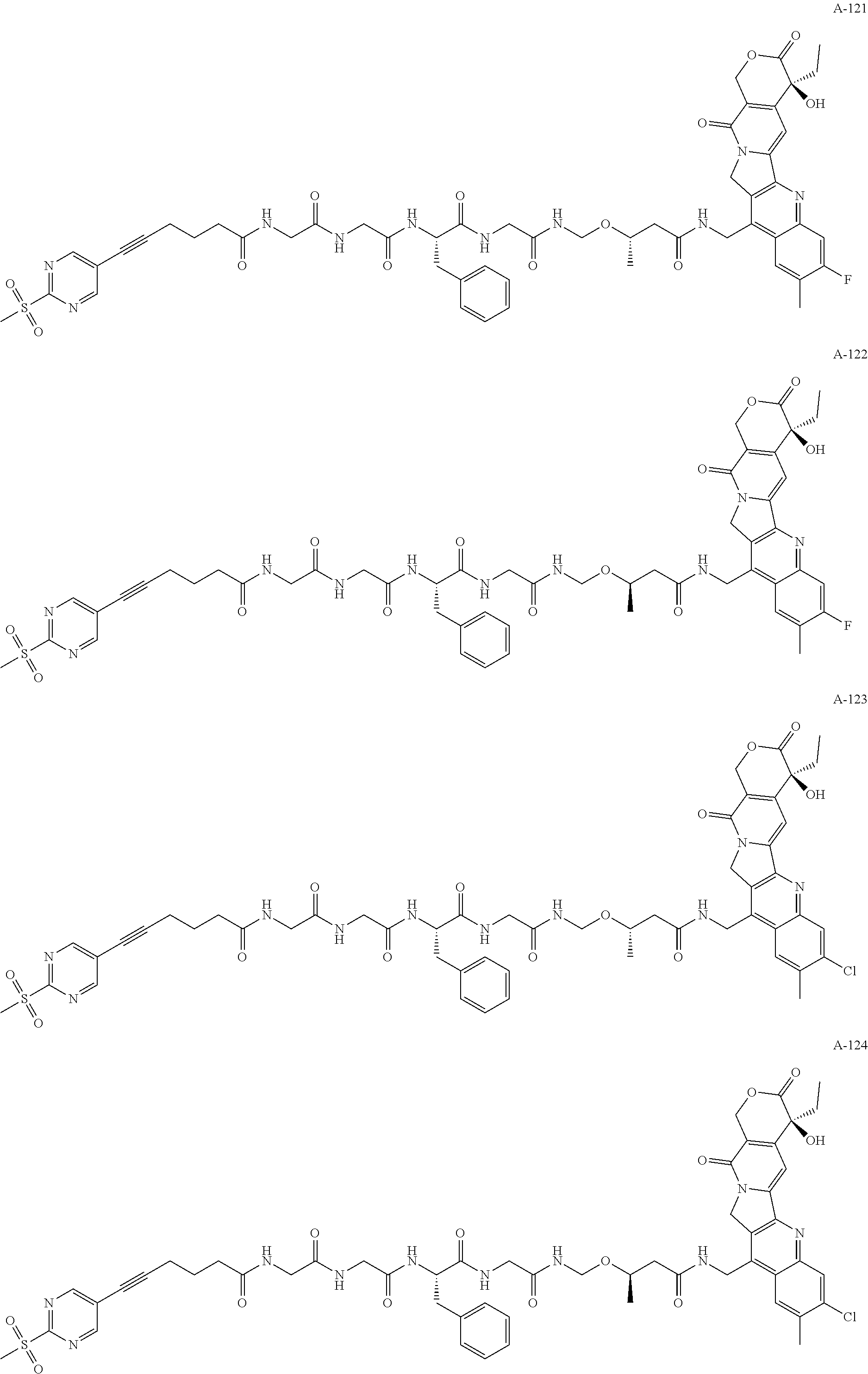

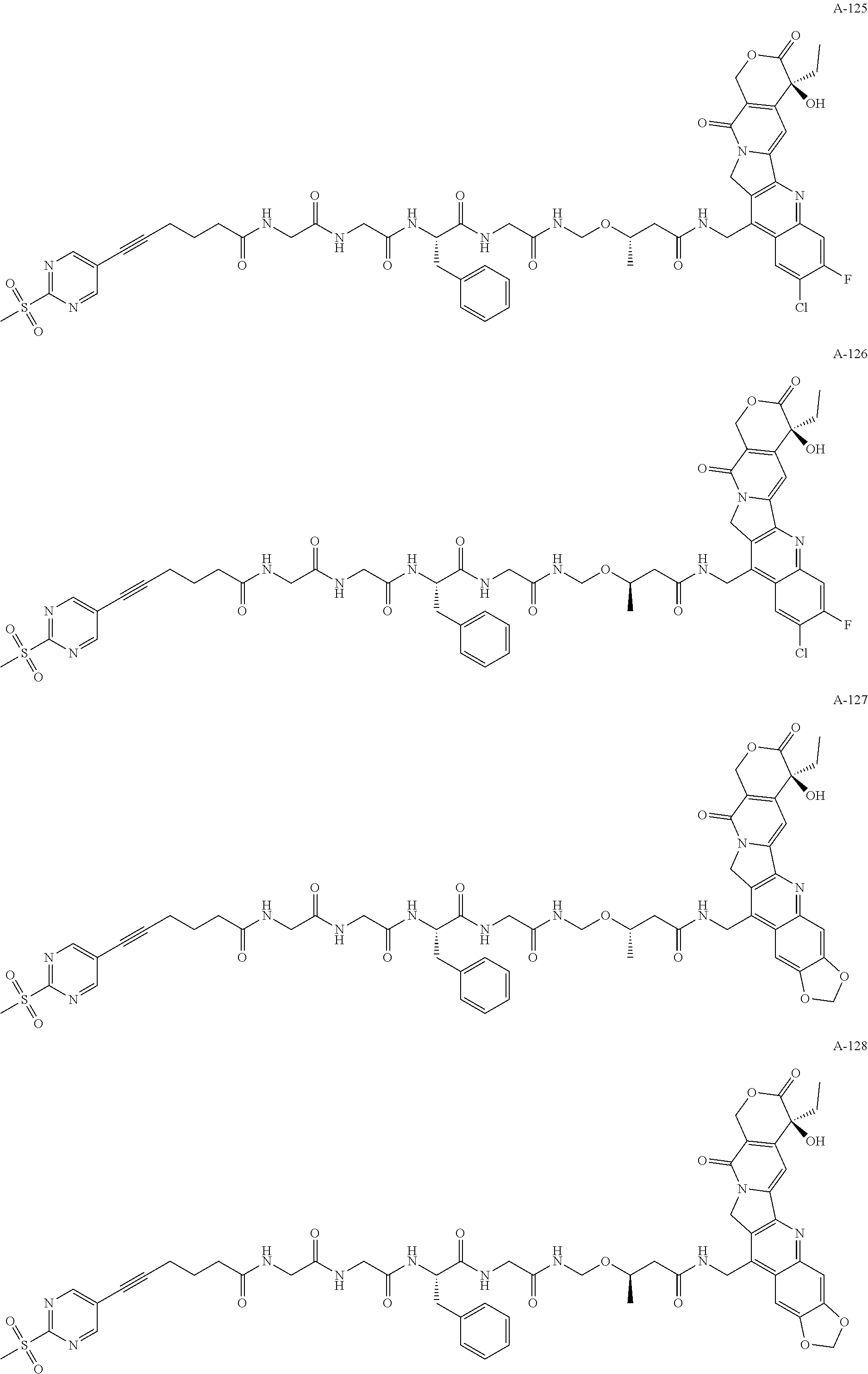

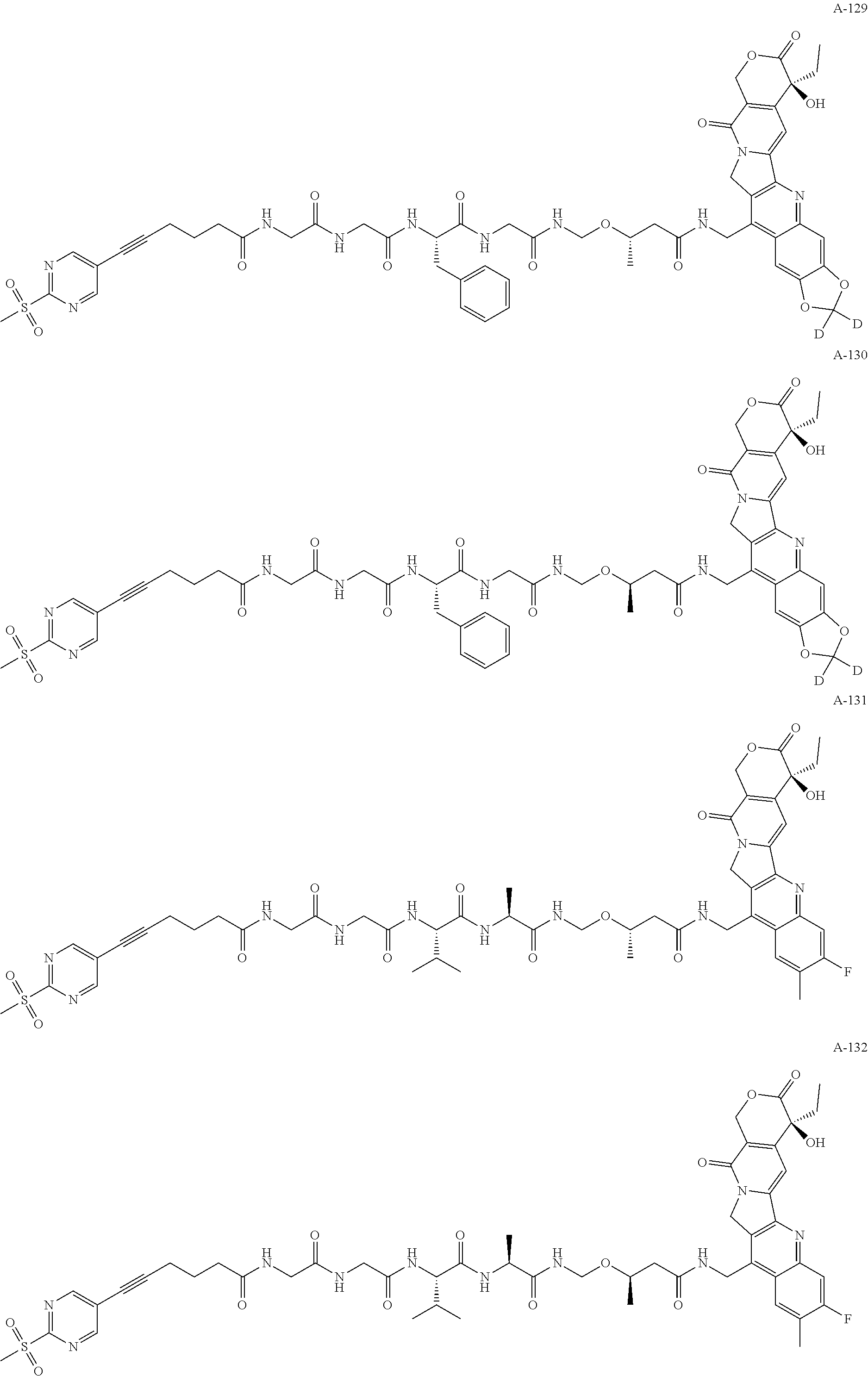

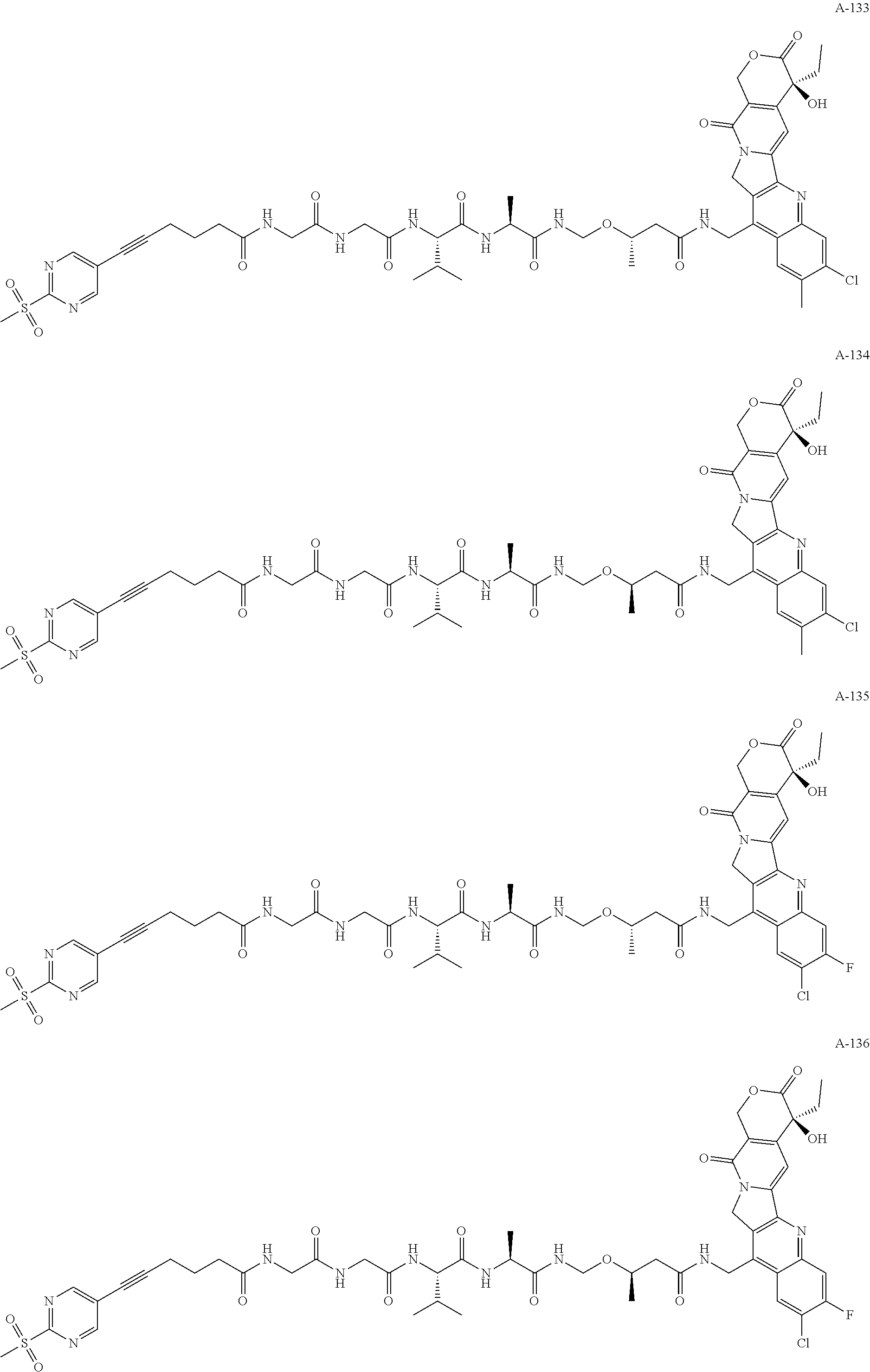

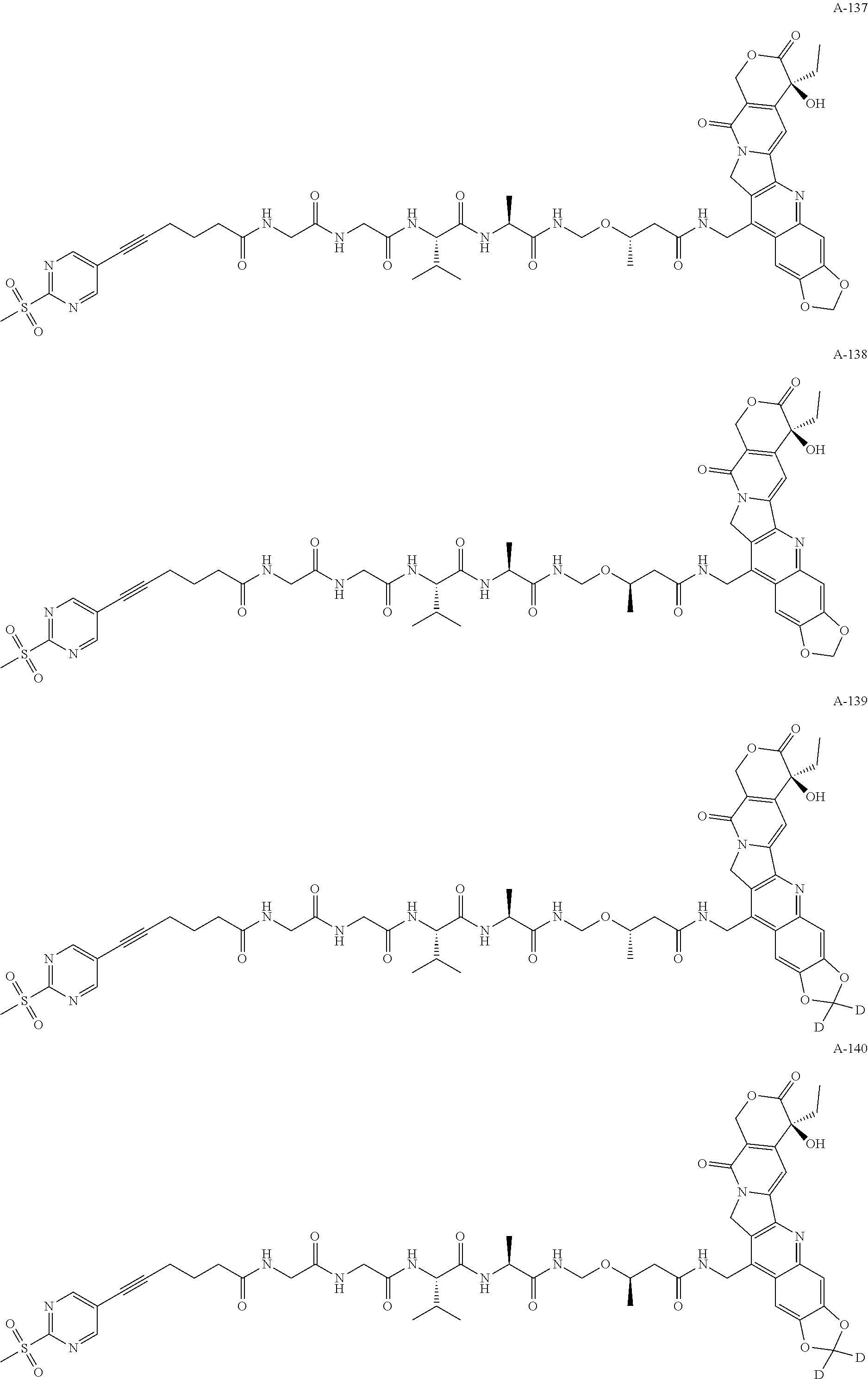

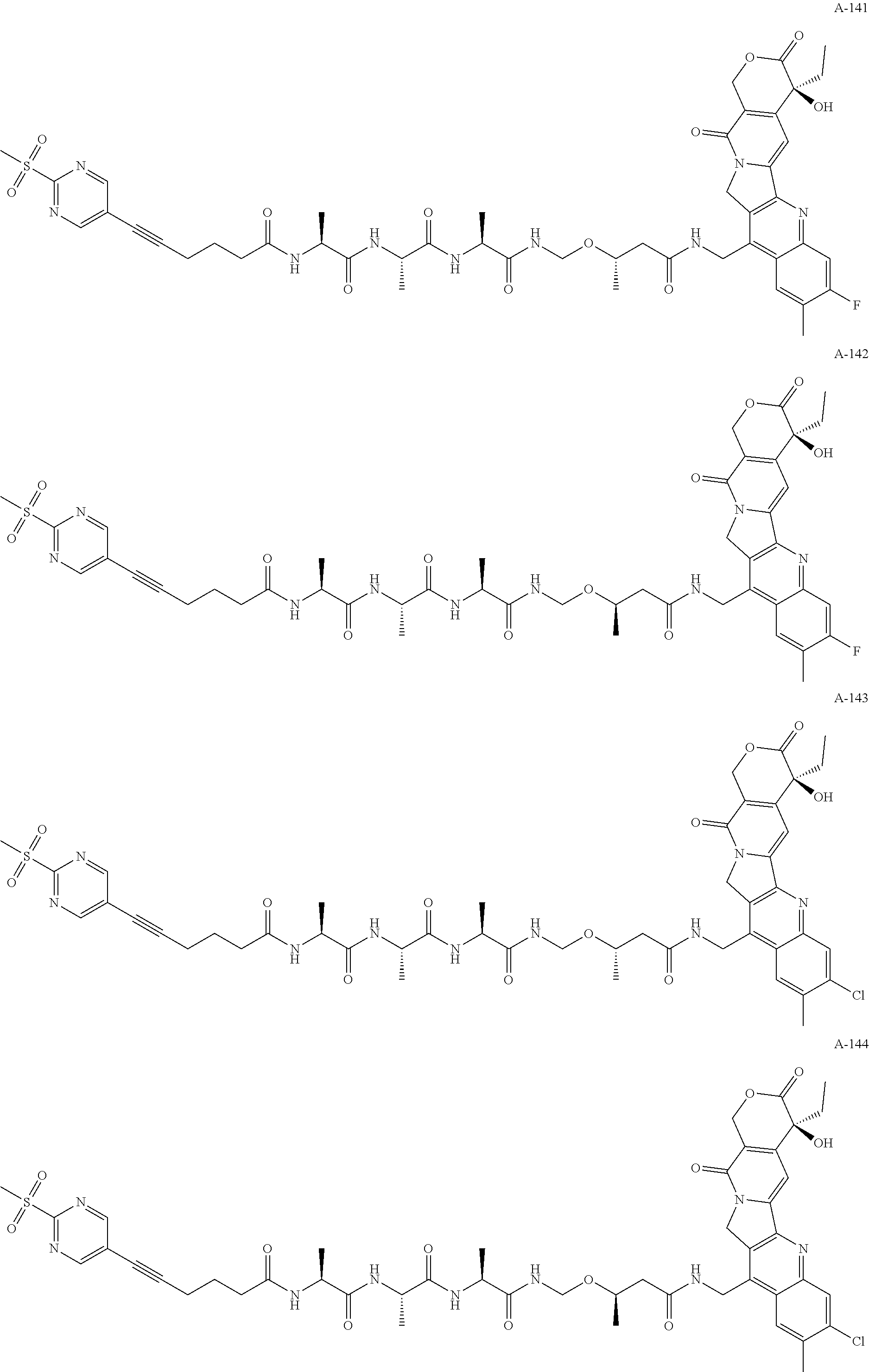

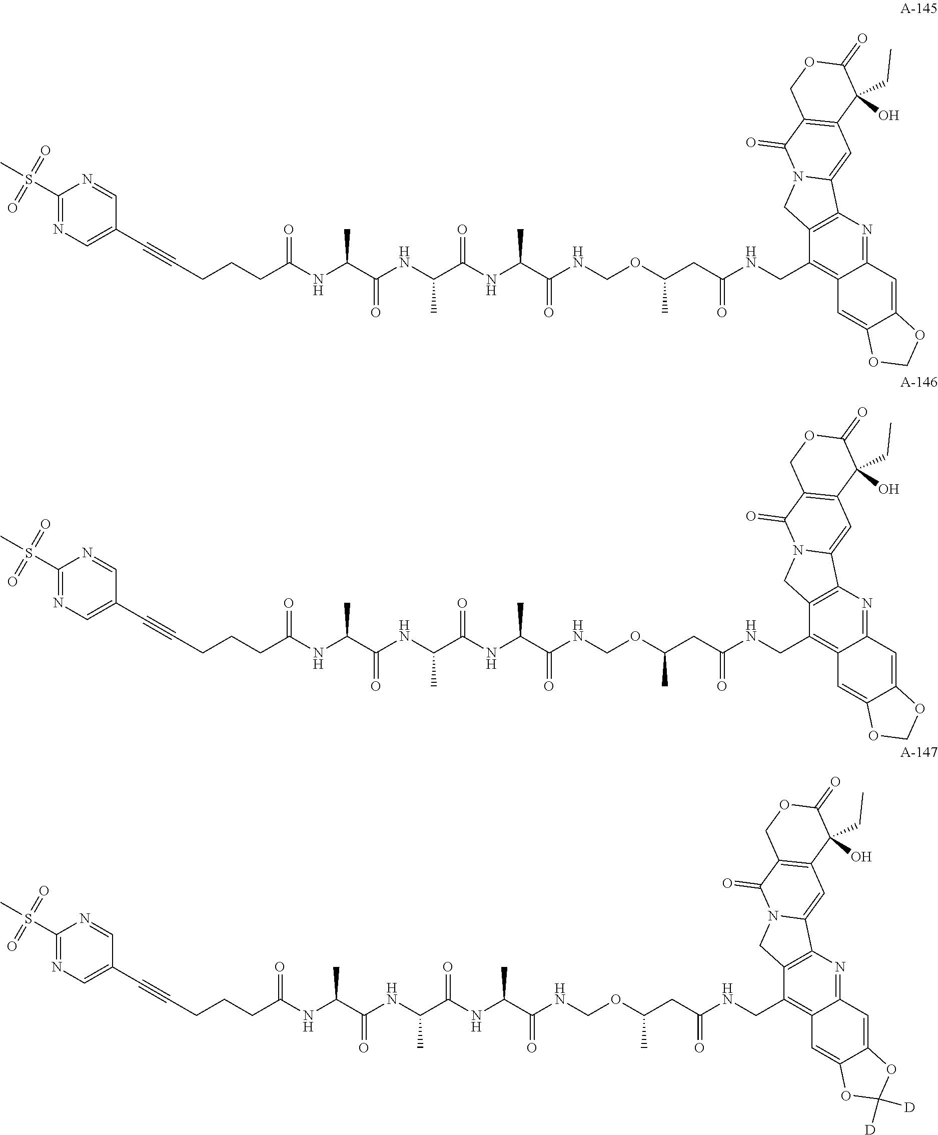

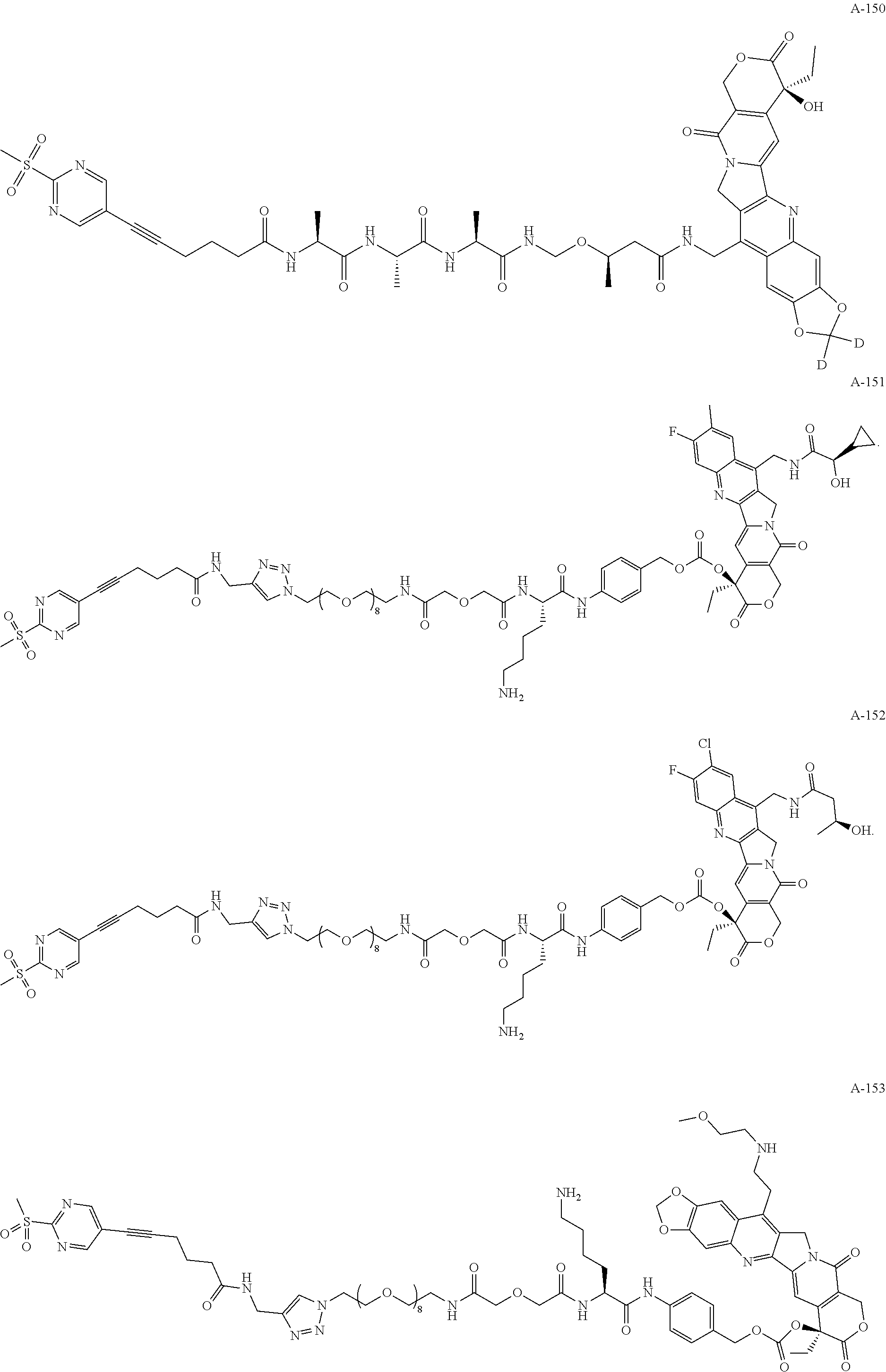

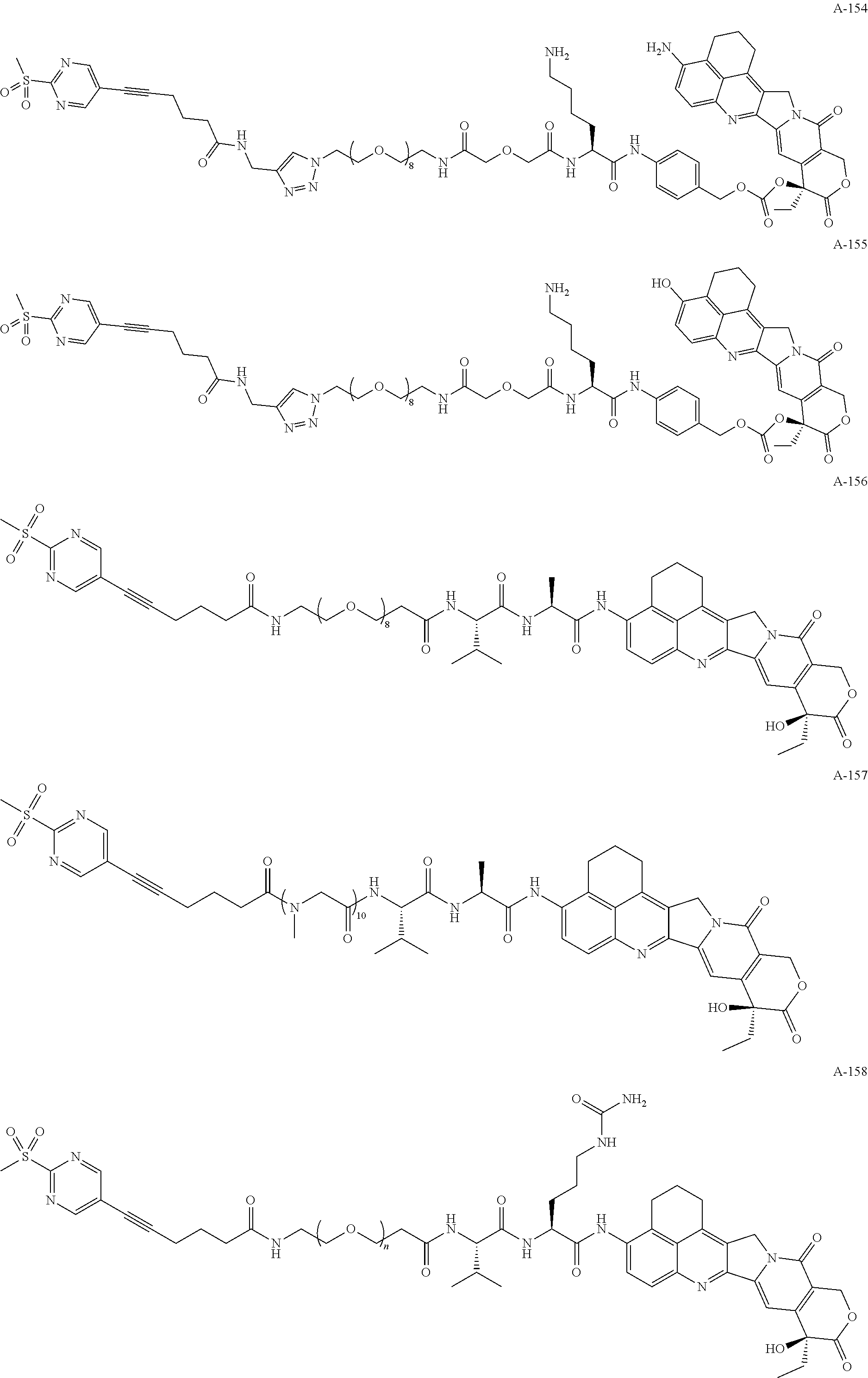

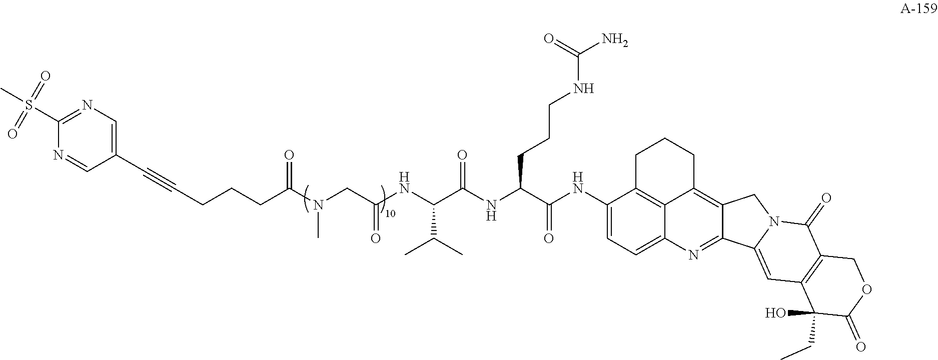

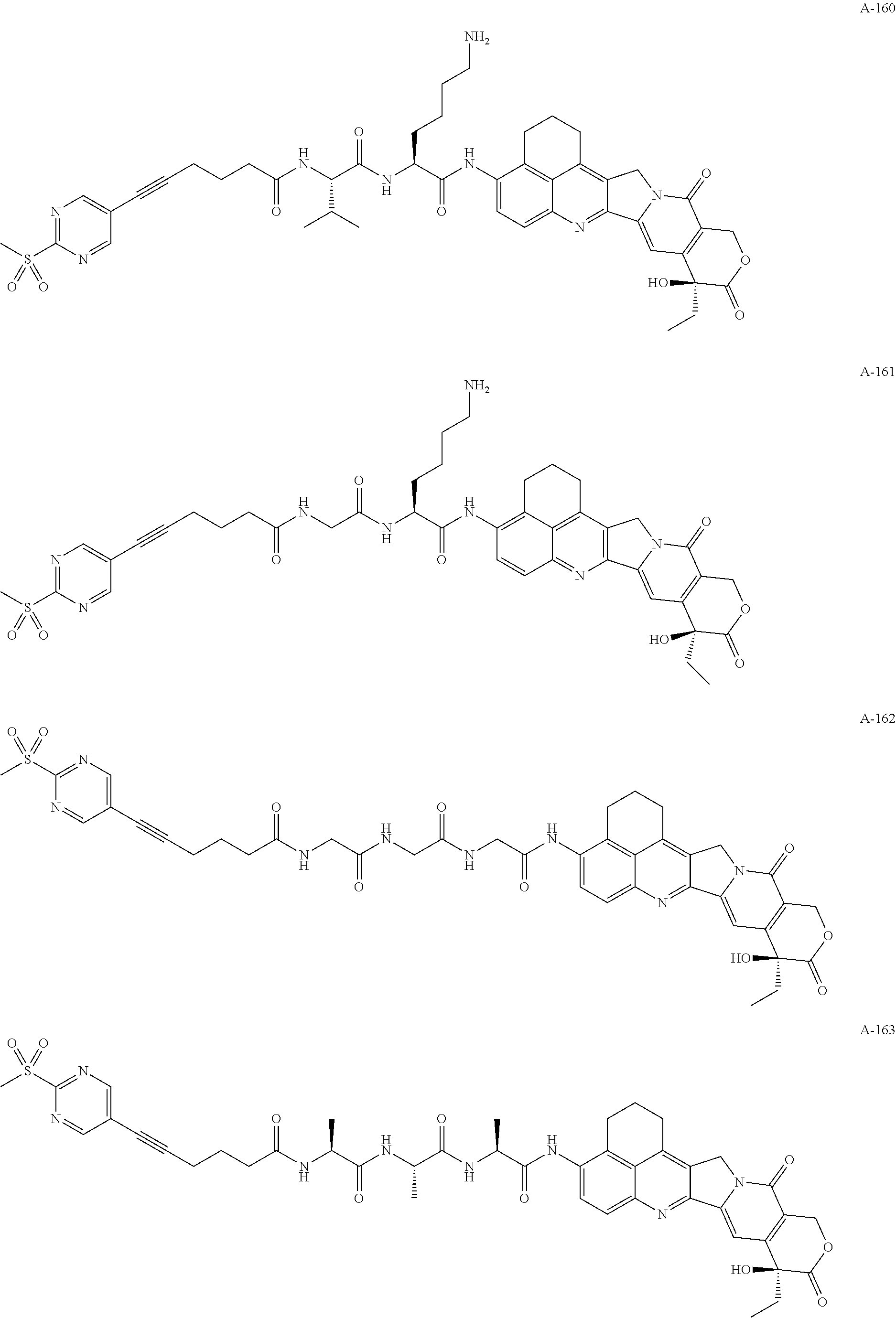

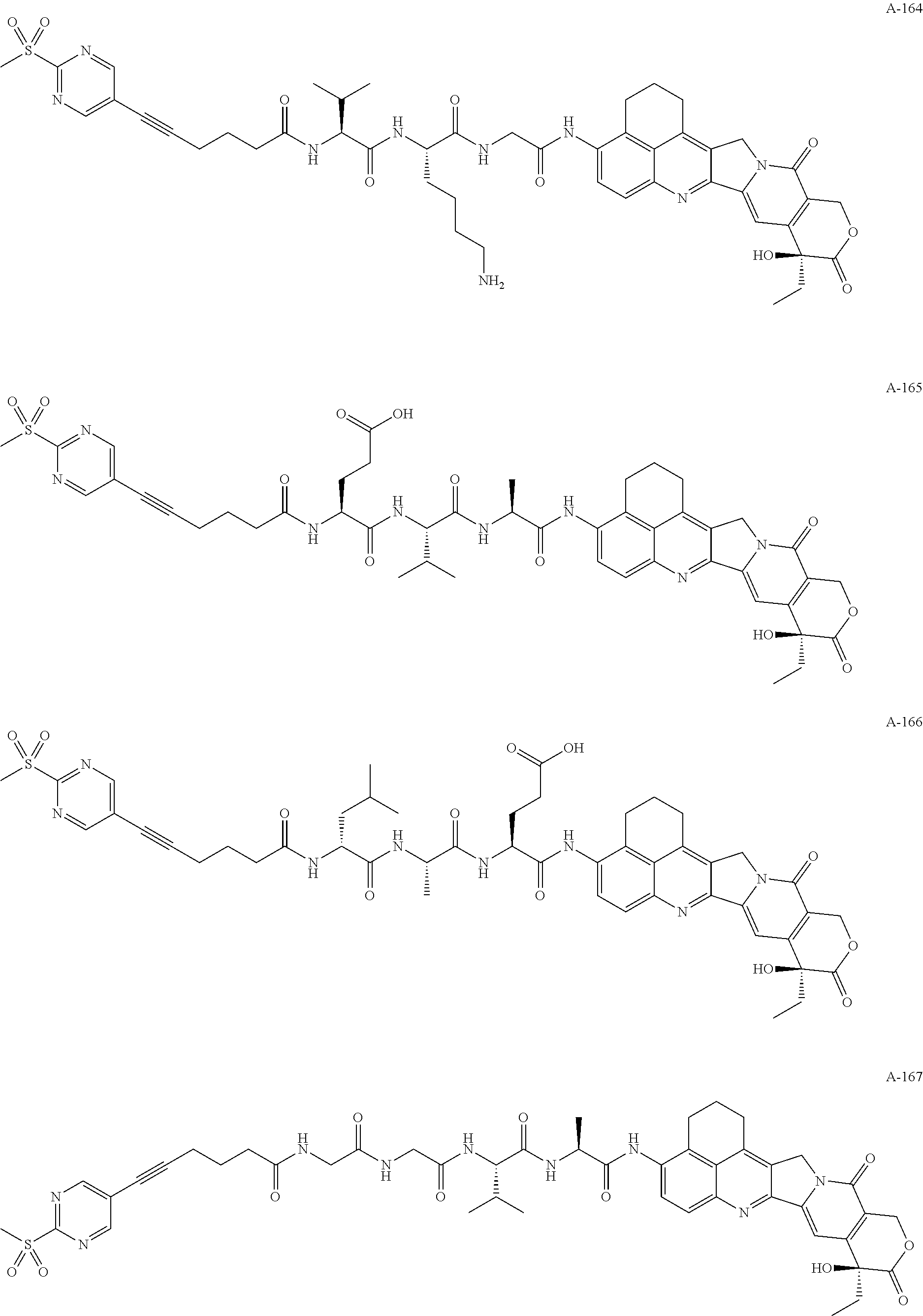

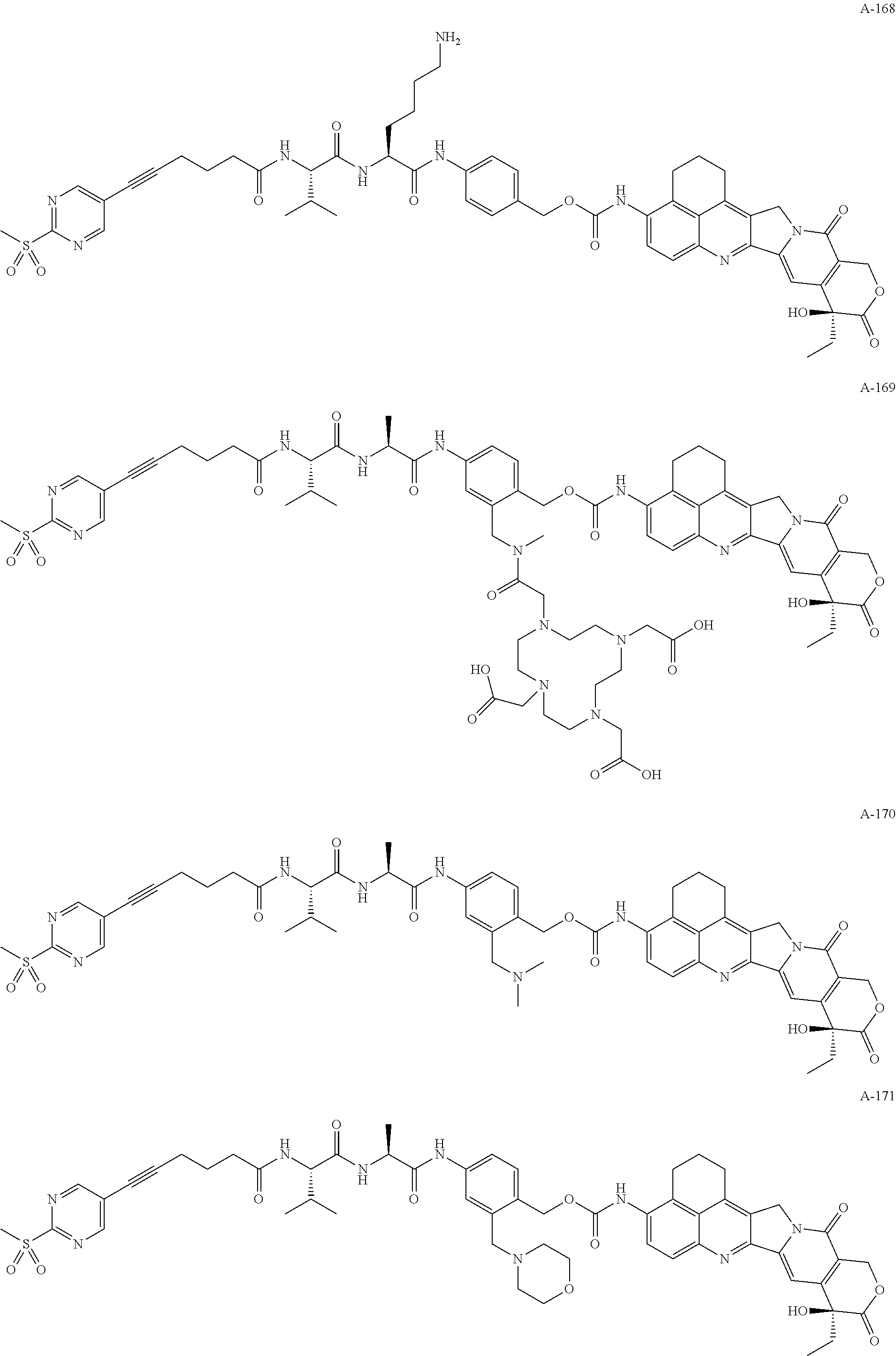

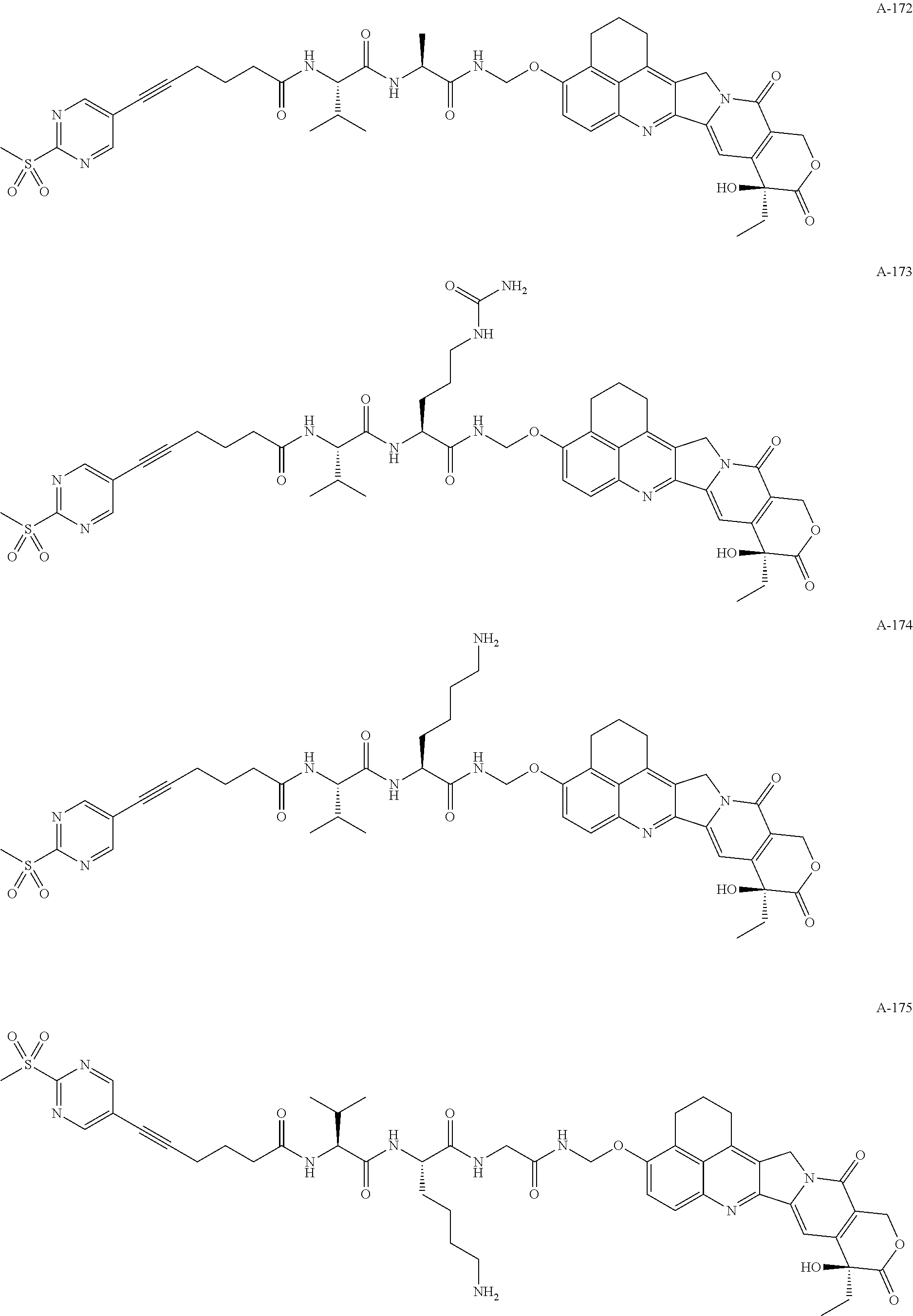

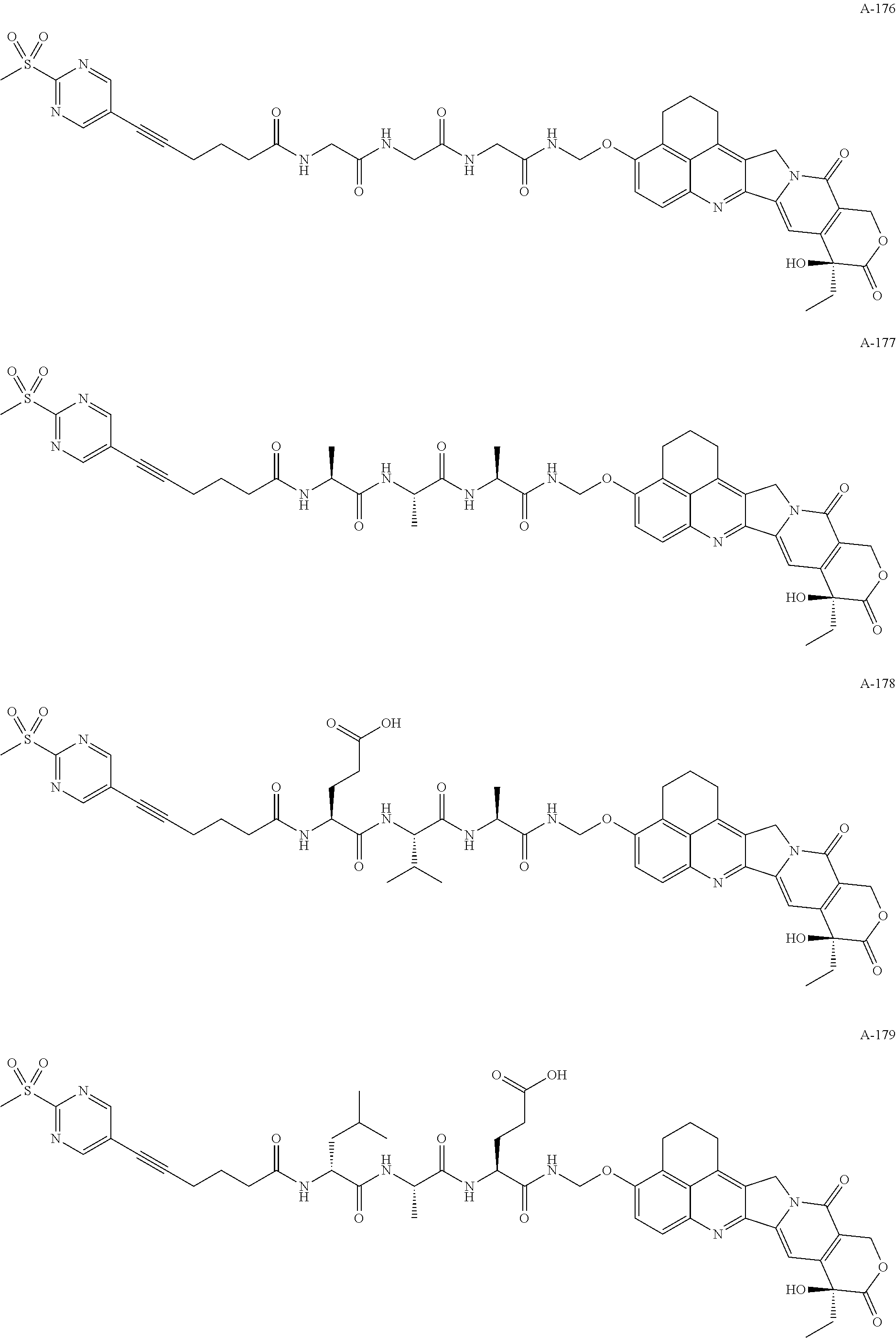

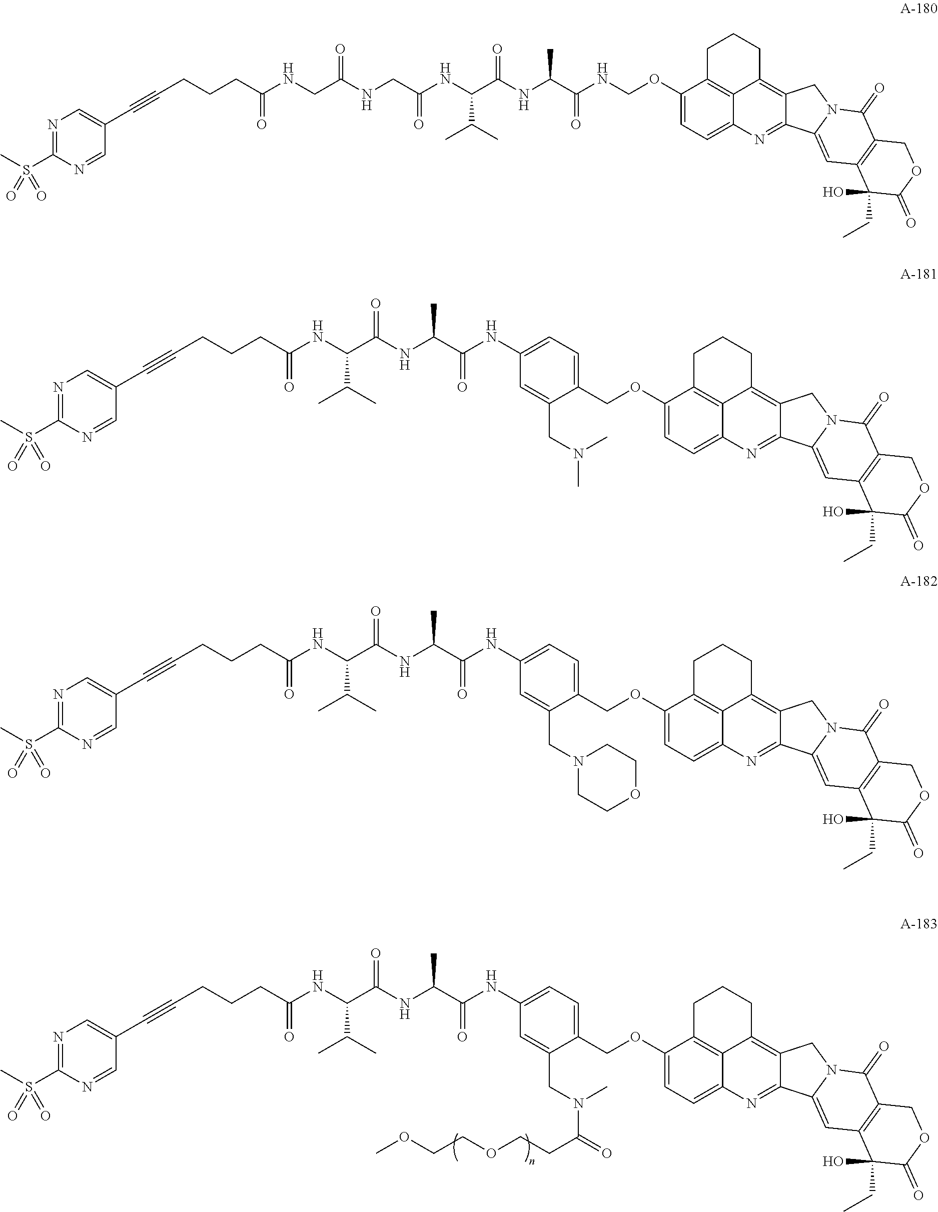

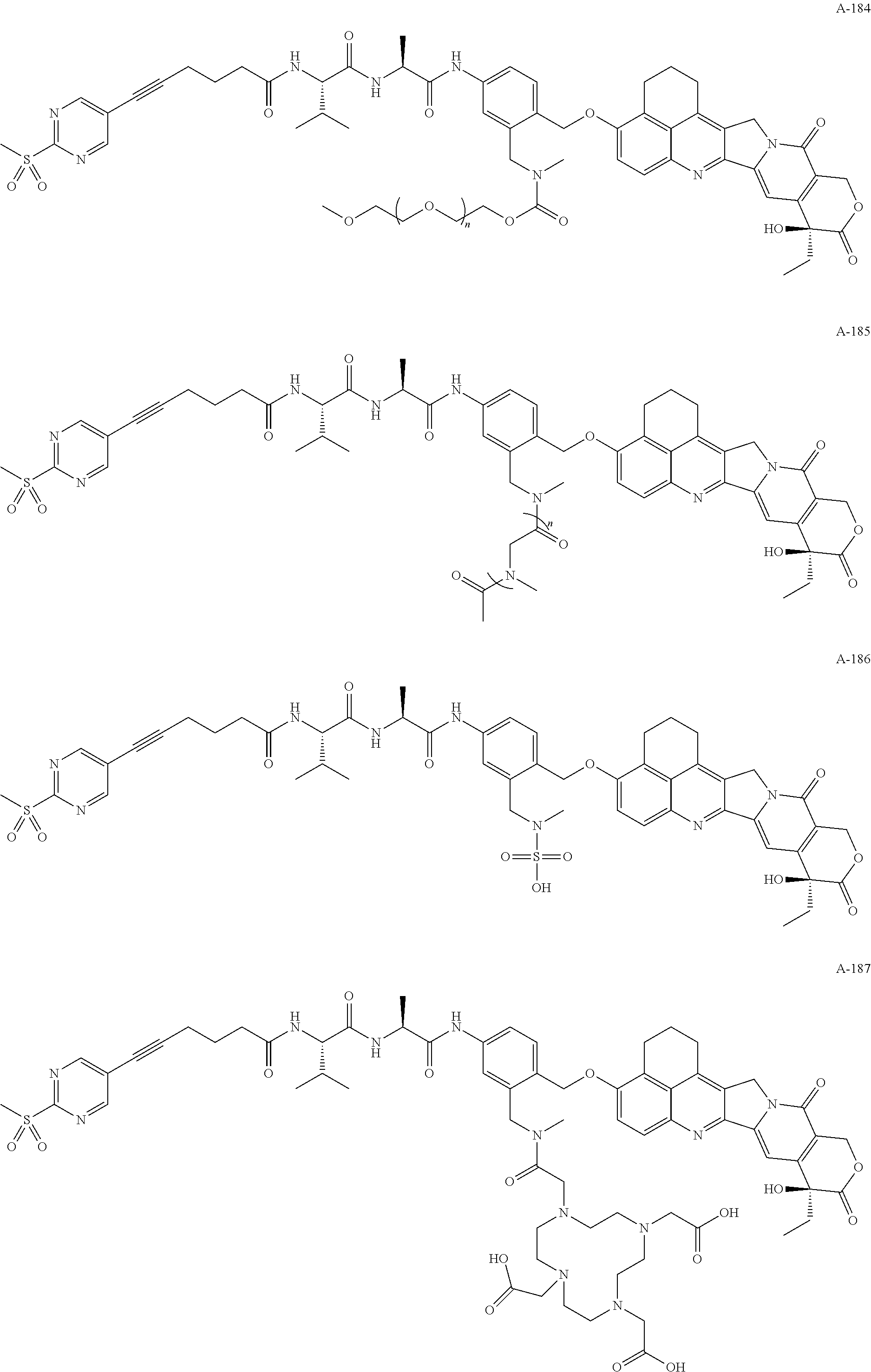

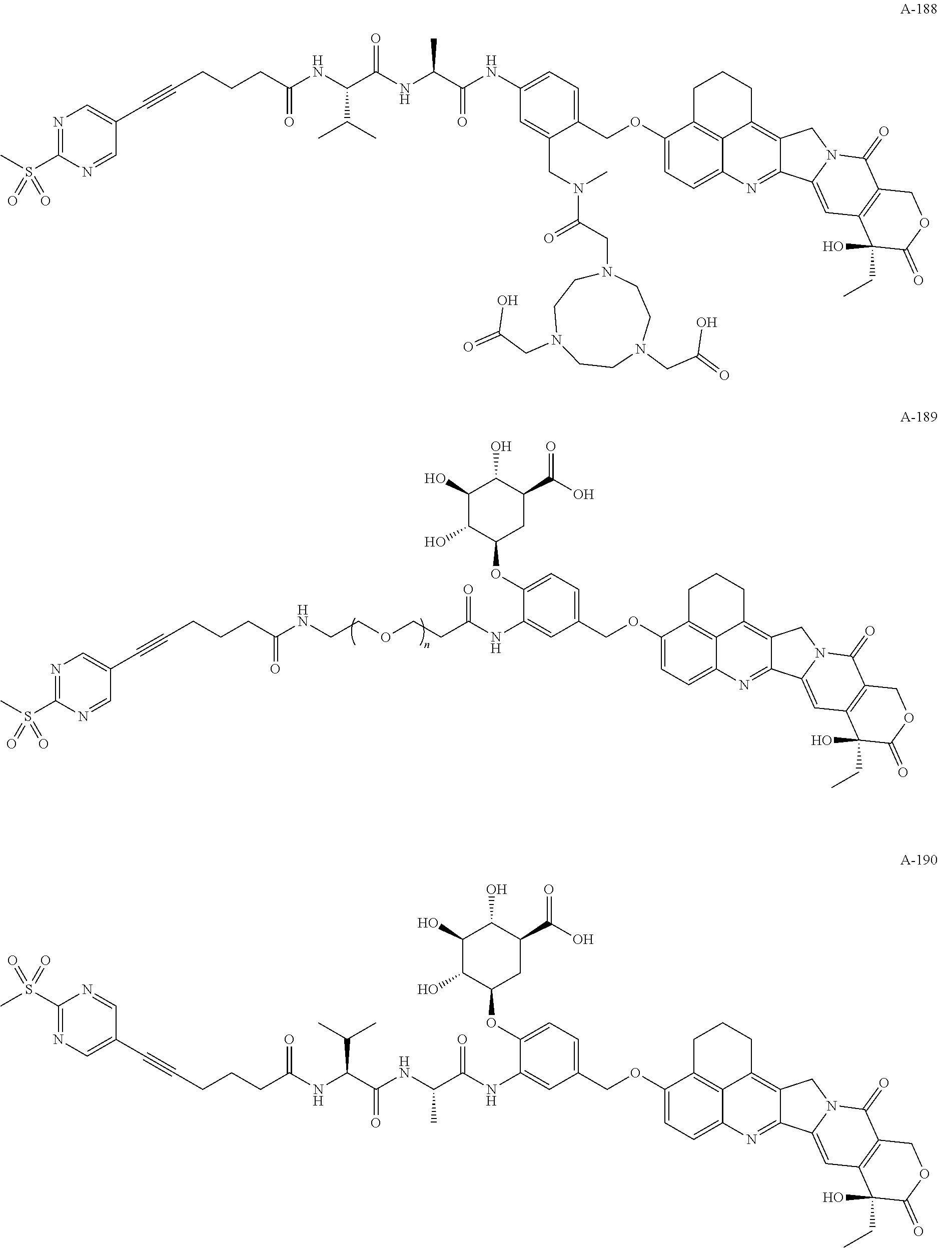

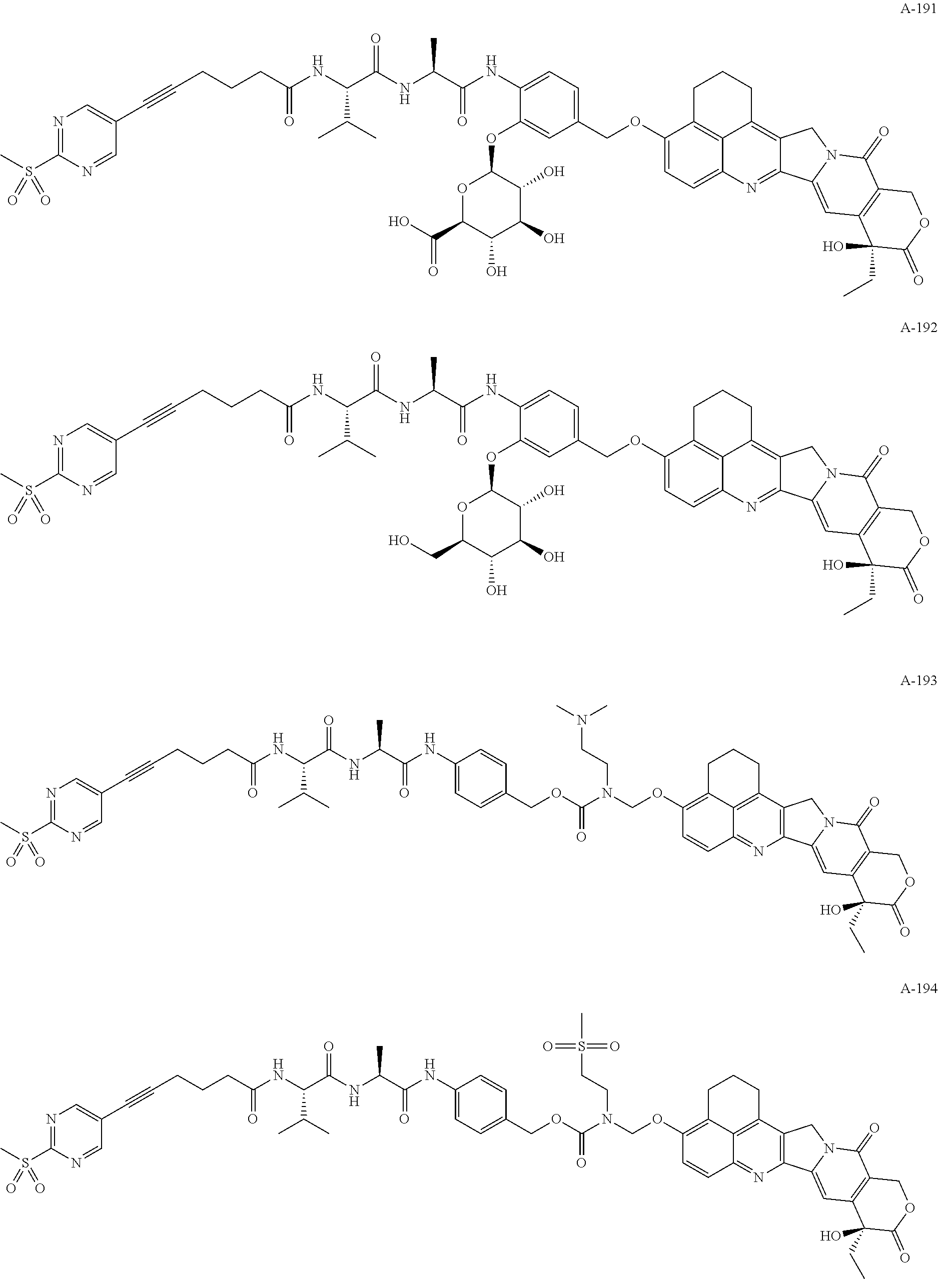

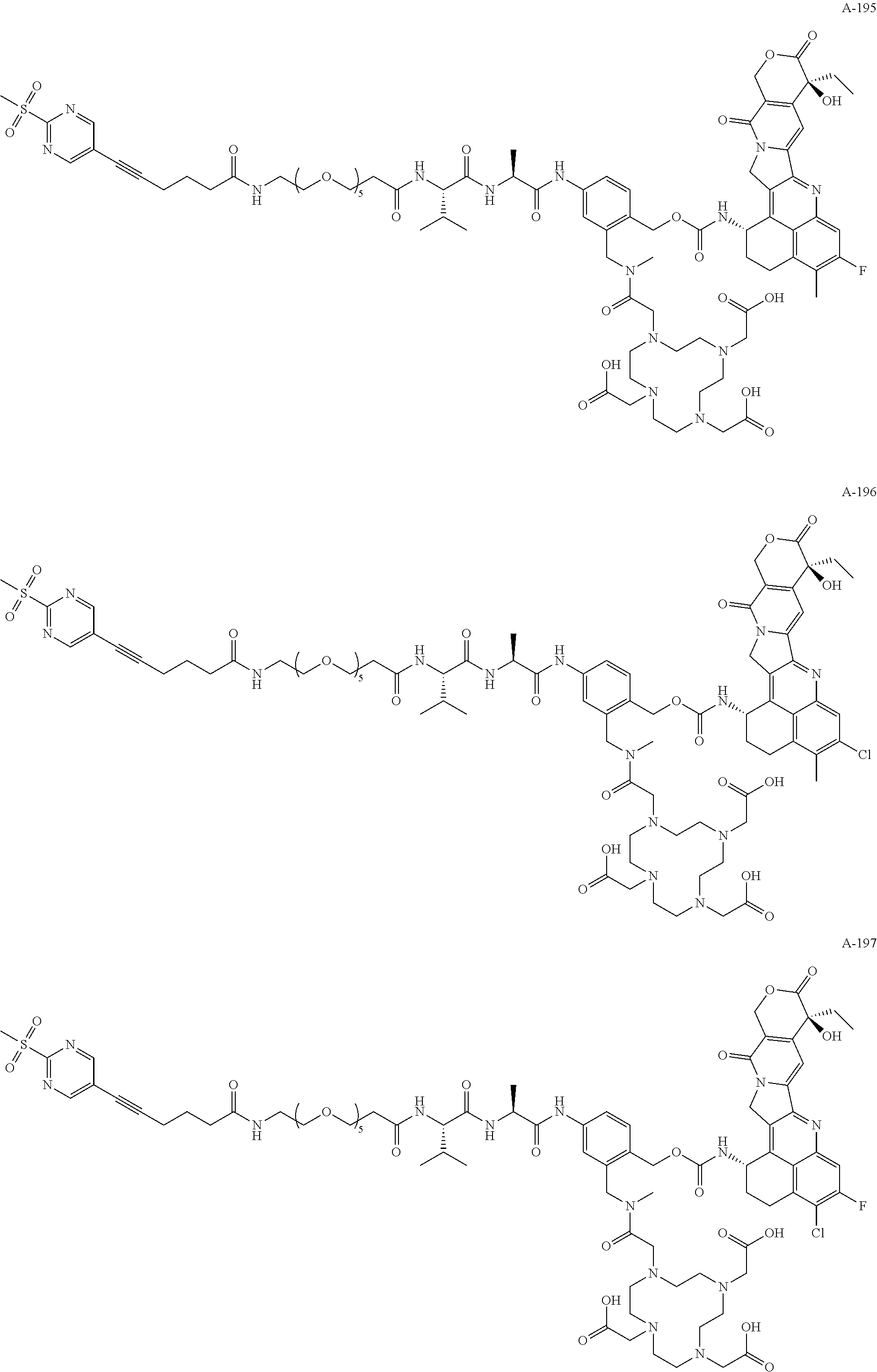

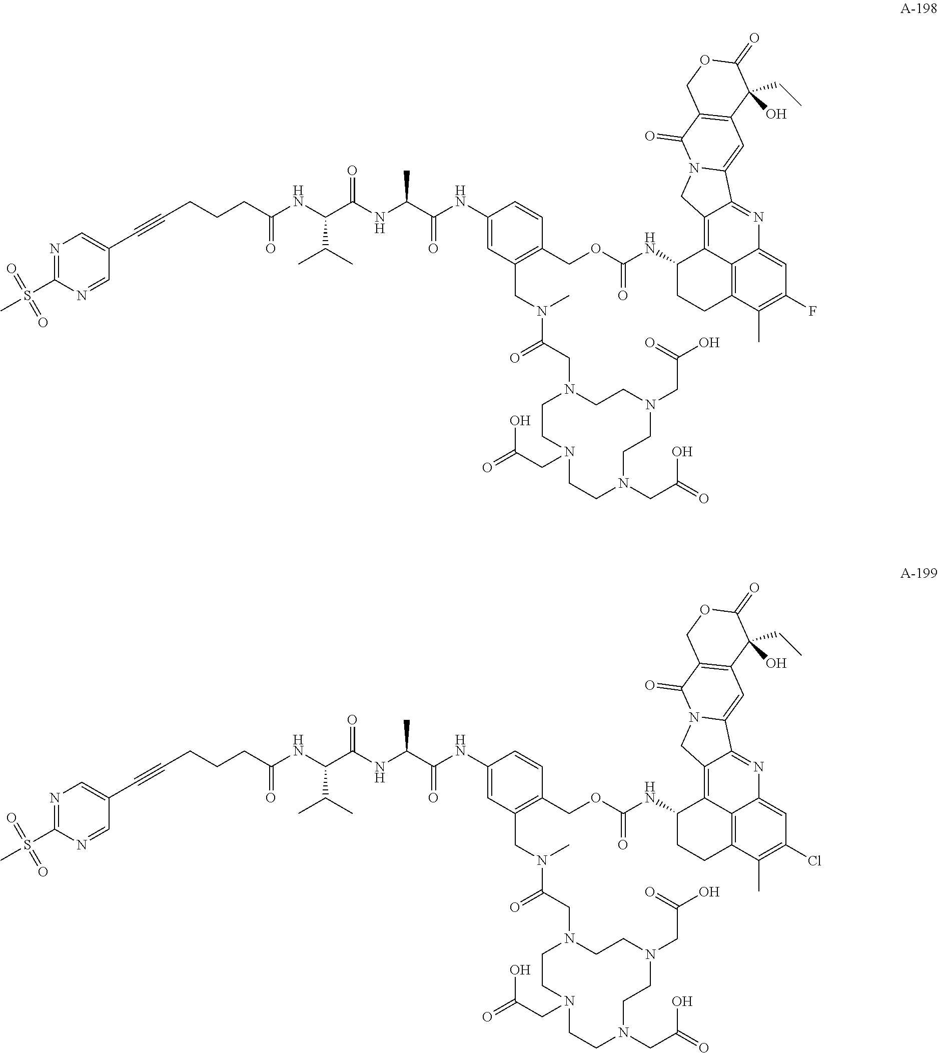

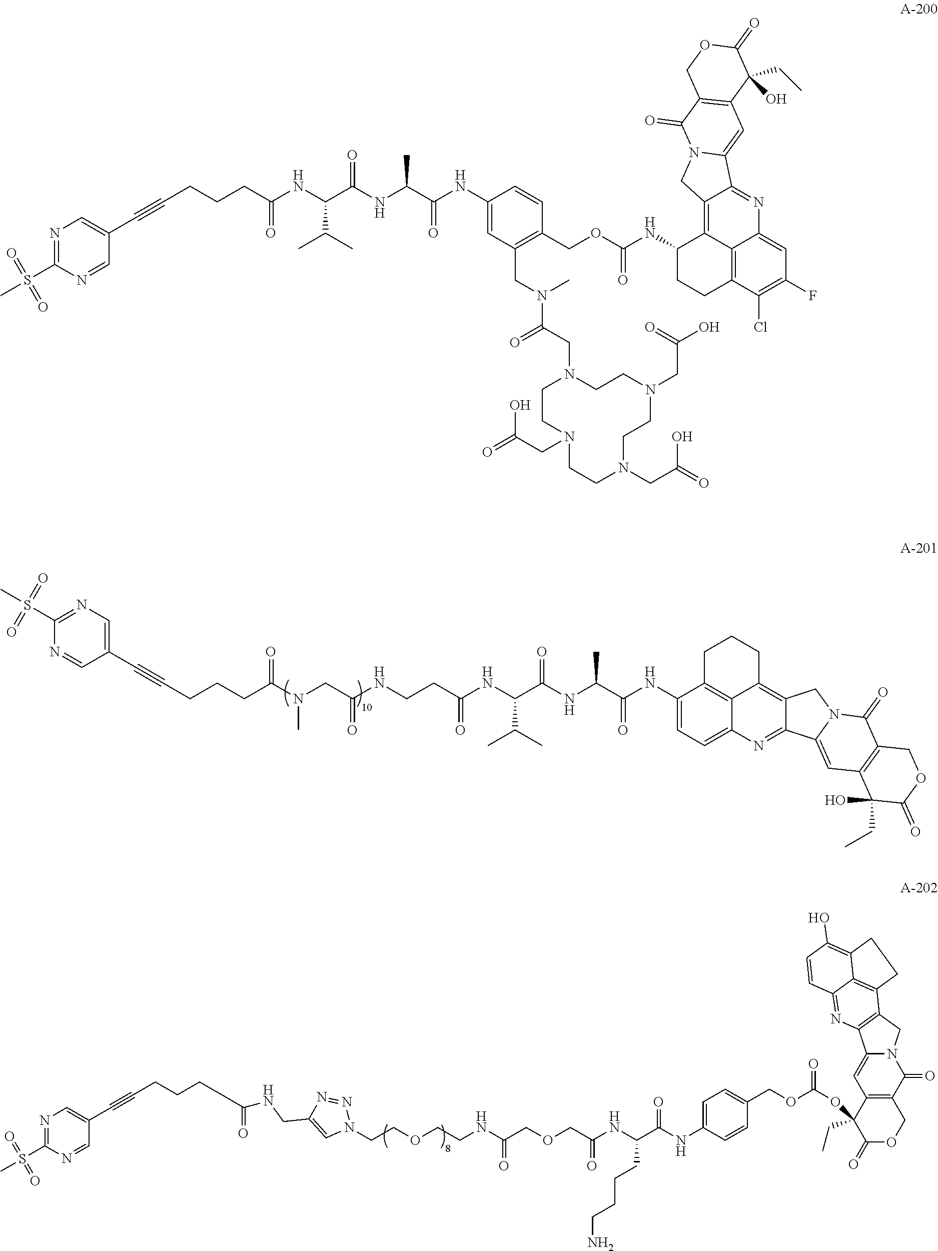

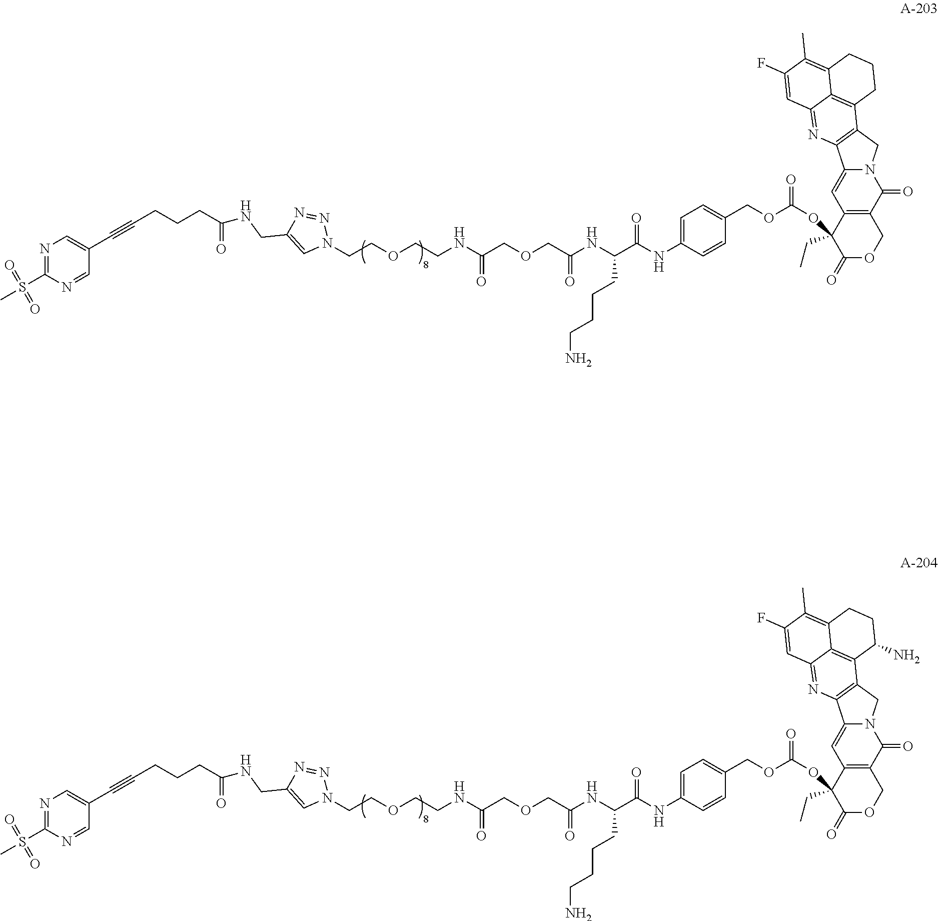

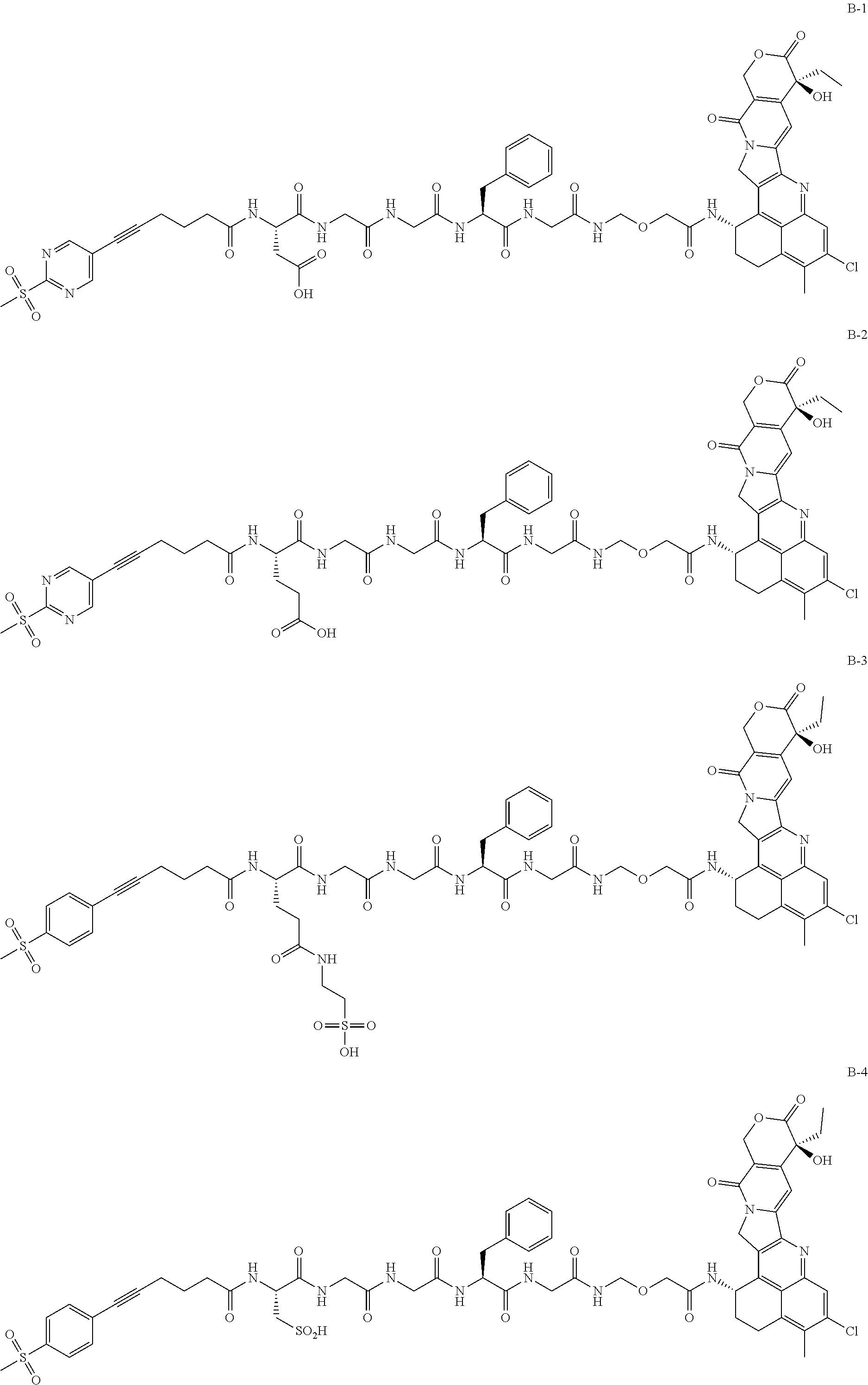

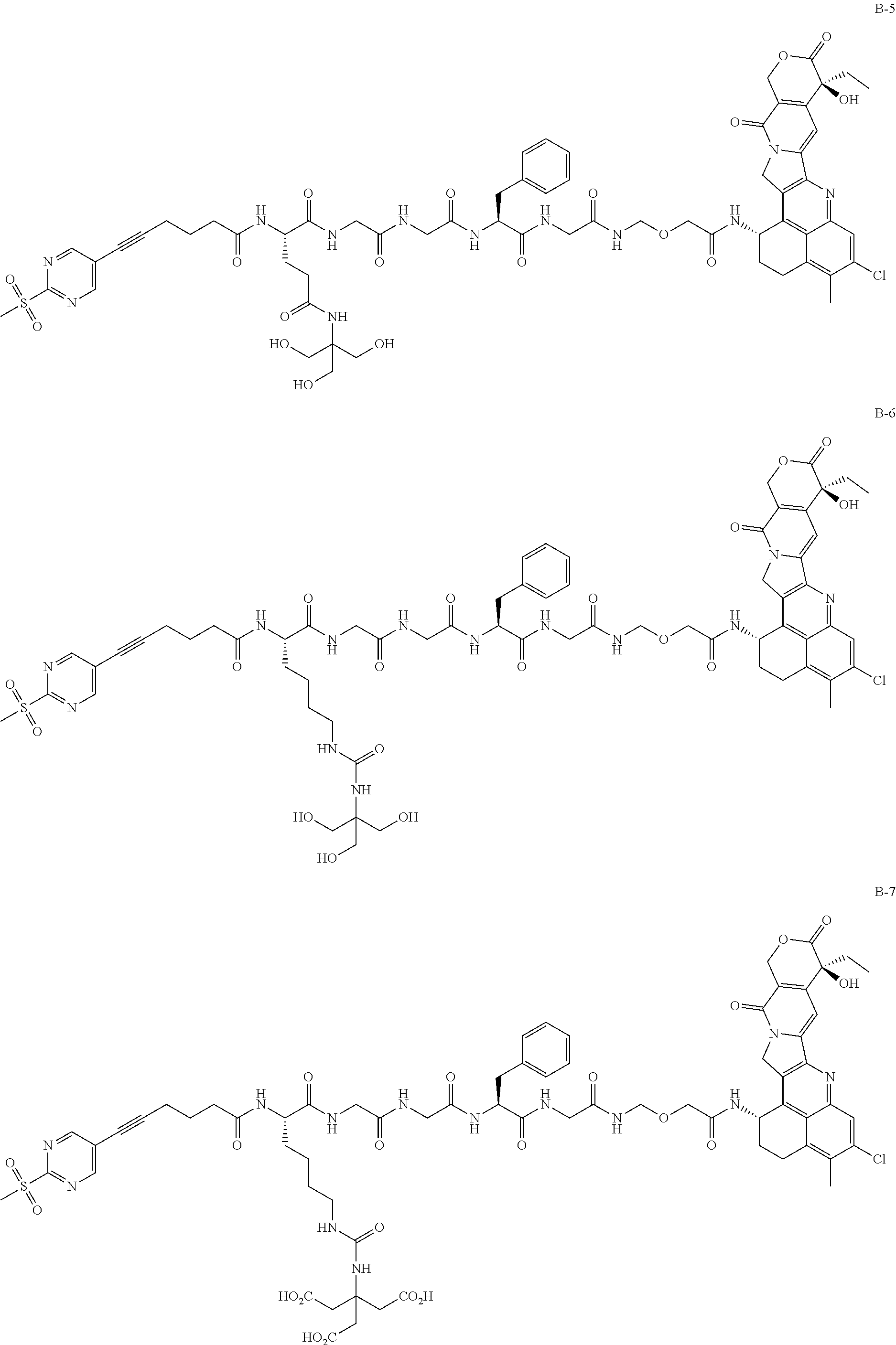

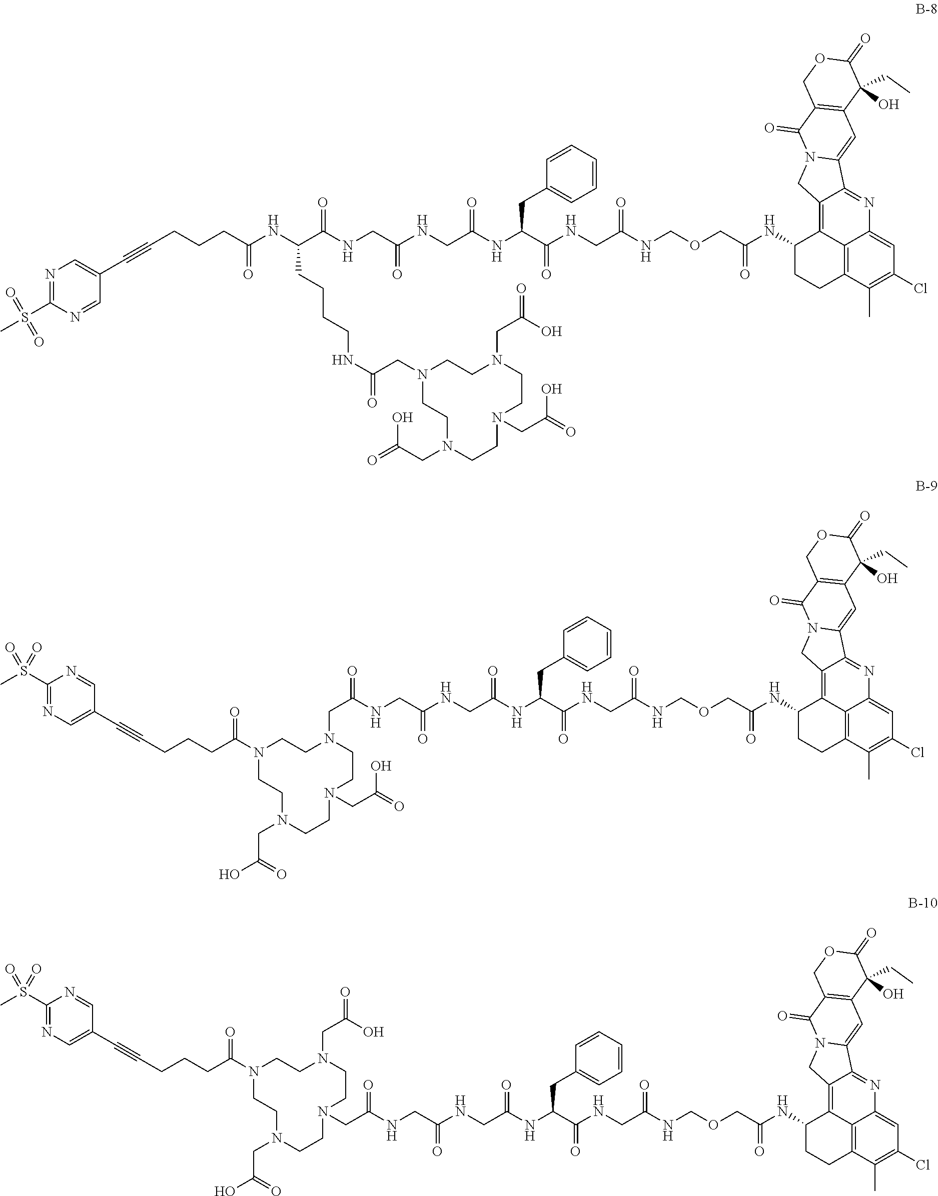

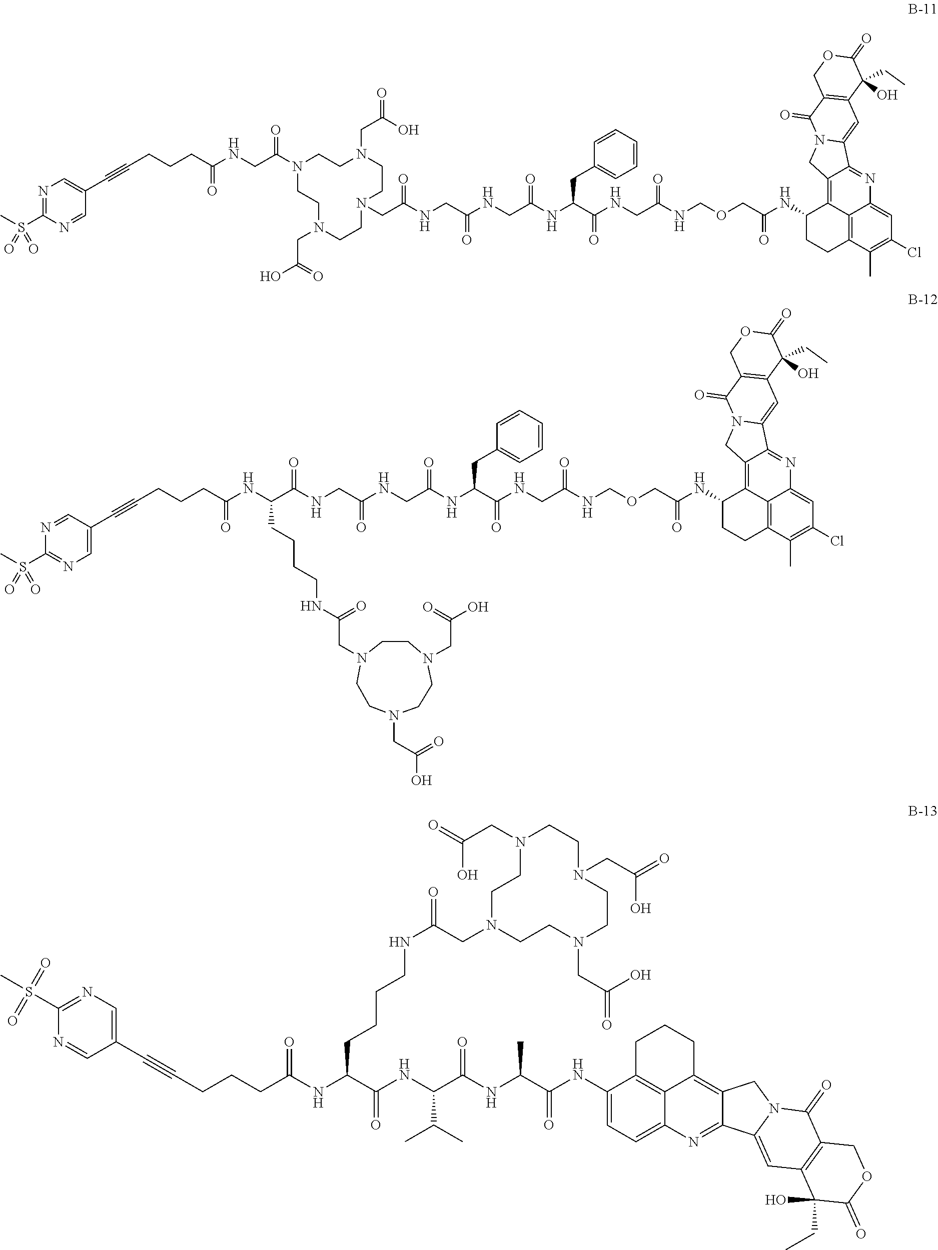

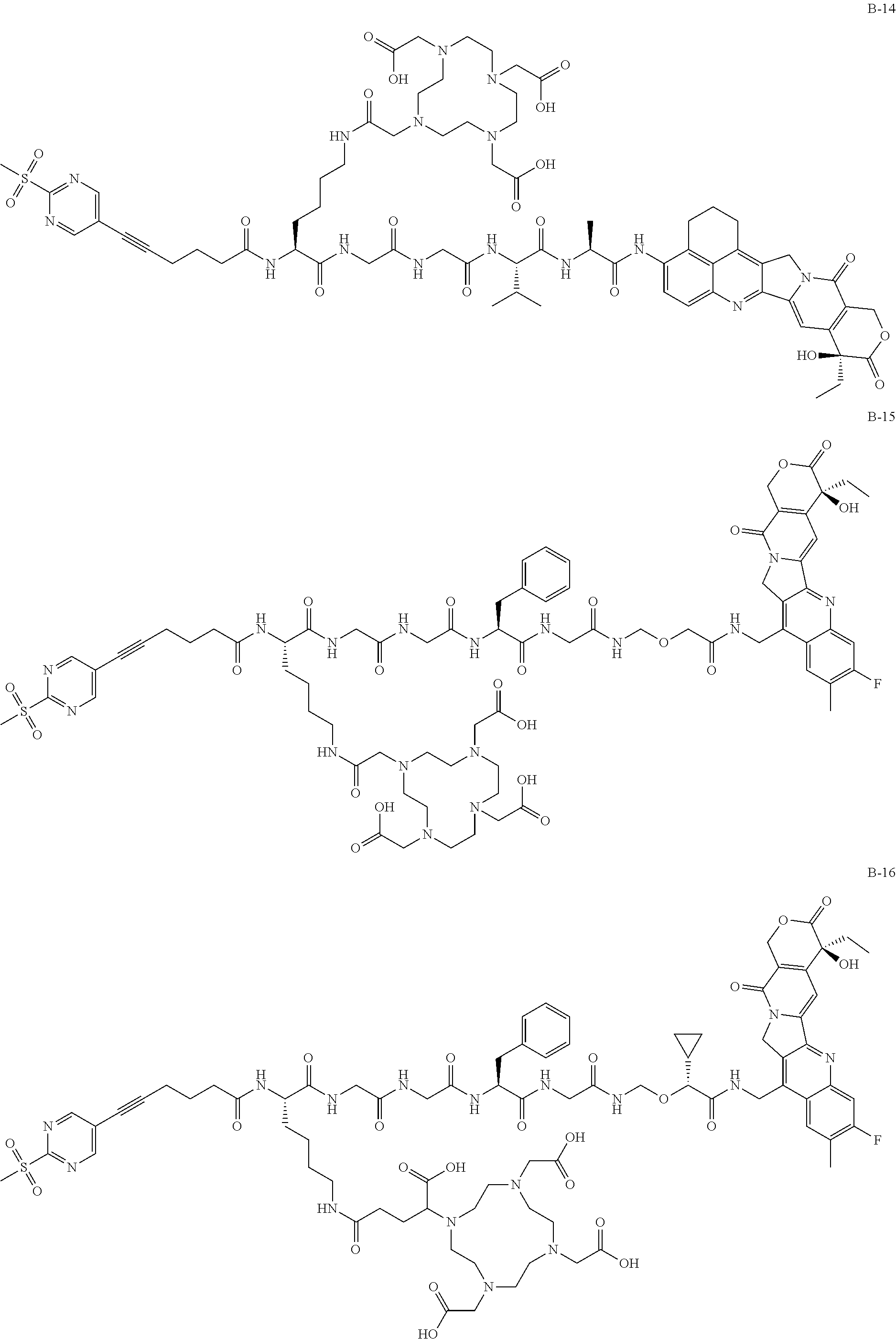

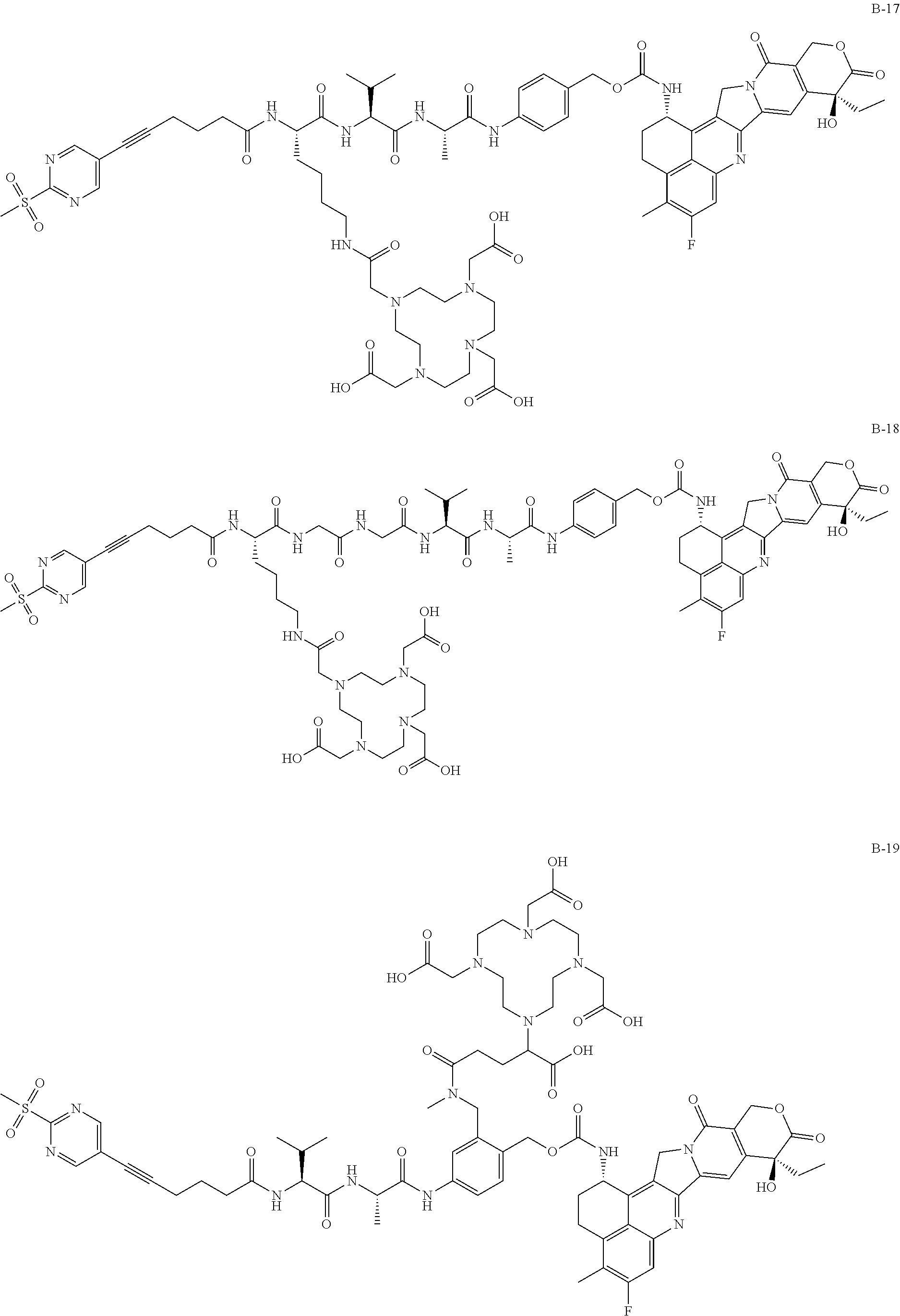

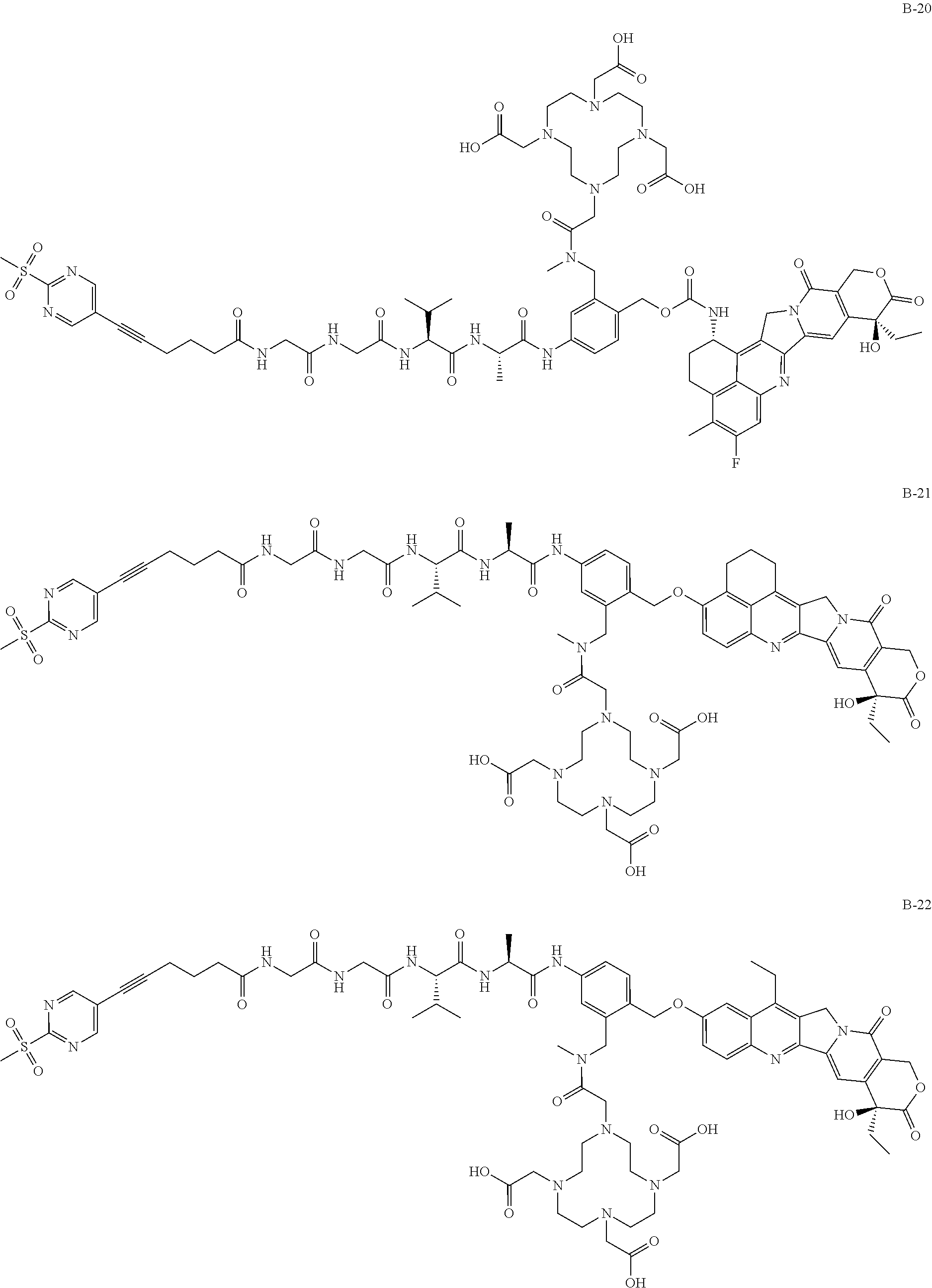

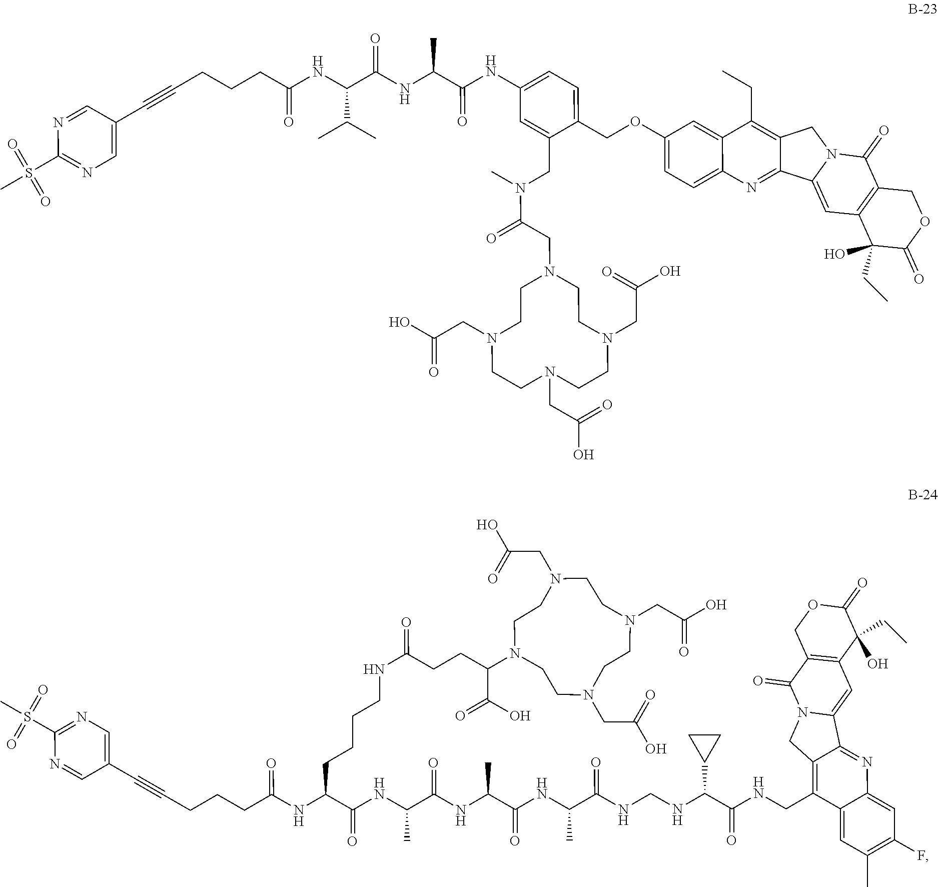

wherein, n is an integer selected from 1 to 20, such as an integer of 1 to 12, 3 to 12, 5 to 15, 5 to 10, such as 1, 2, 3, 4, 5, 6, 7, 8, 9, 10, 11, 12, 13, 14, 15, 16, 17, 18, 19 or 20.

In some embodiments, the compound or pharmaceutically acceptable salt thereof as described above may be optionally substituted with one or more suitable substituents.

### Conjugate

The second aspect of the present invention provides a conjugate represented by Formula (II), wherein:

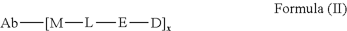

Ab represented a targeting moiety; M, L, E and D are as described in any one of the first aspects of the present invention;
x is 1 to 10.

In some preferred embodiments, the target of Ab is selected from the group consisting of epidermal growth factor, Trop-2, CD37, HER2, CD70, EGFRvIII, Mesothelin, Folate eceoptor1, Mucin 1, CD138, CD20, CD19, CD30, SLTRK6, Nectin 4, Tissue factor, Mucin16, Endothelin receptor, STEAP1, SLC39A6, Guanylylcyclase C, PSMA, CCD79b, CD22, Sodium phosphate cotransporter 2B, GPNMB, Trophoblast glycoprotein, AGS-16, EGFR, CD33, CD66e, CD74, CD56, PD-L1, TACSTD2, DR5, E16, STEAP1, 0772P, MPF, Napi3b, Sema 5b, PSCA hlg, ETBR, MSG783, STEAP2, TrpM4, CRIPTO, CD21, CD79b, FcRH2, NCA, MDP, IL20Rα, Brevican, EphB2R, ASLG659, PSCA, GEDA, BAFF-R, CD22, CD79a, CXCR5, HLA-DOB, P2X5, CD72, LY64, FcRH1, IRTA2, TENB2, integrin α5β6, α4β7, FGF2, FGFR2, Her3, CD70, CA6, DLL3, DLL4, P-cadherin, EpCAM, pCAD, CD223, LYPD3, LY6E, EFNA4, ROR1, SLITRK6, 5T4, ENPP3, SLC39A6, Claudin18.2, BMPR1B, E16, STEAP1, Tyro7, 0772P, MPF, Napi3b, Sema 5b. PSCA hlg, ETBR, MSG783, STEAP2, TrpM4, CRIPTO, CD21, CD79b, FcRH2, NCA, MDP, IL20Rα, Brevican, EphB2R, ASLG659, PSCA, GEDA, CD22, CD79a, CXCR5, HLA-DOB, P2X5, CD72, LY64, FcRH1, IRTA2, c-Met, ApoE, CD1 lc, CD40, CD45 (PTPRC), CD49D (ITGA4), CD80, CSF1R, CTSD, GZMB, Ly86, MS4A7, PIK3AP1, PIK3CD, CCR5, IFNG, IL10RA1, IL-6, ACTA2, COL7A1, LOX, LRRC15, MCPT8, MMP10, NOG, SERPINE1, STAT1, TGFBR1, CTSS, PGF, VEGFA, C1QA, C1QB, ANGPTL4, EGLN, ANGPTL4, EGLN3, BNIP3, AIF1, CCL5, CXCL10, CXCL11, IF16, PLOD2, KISS1R, STC2, DDIT4, PFKFB3, PGK1, PDK1, AKR1C1, AKR1C2, CADM1, CDH11, COL6A3, CTGF, HMOX1, KRT33A, LUM, WNT5A, IGFBP3, MMP14, CDCP1, PDGFRA, TCF4, TGF, TGFB1, TGFB2, CDl lb, ADGRE1, EMR2, TNFRSF21, UPK1B, TNFSF9, MMP16, MFI2, IGF-1R, RNF43, NaPi2b and TENB2.

In some preferred embodiments, Ab is a small molecule ligand, such as a folic acid derivative, a glutamate urea derivative, a somatostatin derivative, an aromatic sulfonamide derivative (e.g., a carbonic anhydrase IX inhibitor), a polyene connecting two aliphatic indoles, a cyanine dye, or IR-783 or derivative thereof.

In some preferred embodiments, Ab is an antibody, such as a monoclonal antibody or antigen-binding fragment thereof, wherein the monoclonal antibody or antigen-binding fragment thereof comprises Fab, Fab′, F(ab′)₂, Fd, Fv, dAb, complementary determining region fragment, single chain antibody (e.g., scFv), non-human antibody, humanized antibody, chimeric antibody, fully human antibody, probody, bispecific antibody, or multispecific antibody.

In some preferred embodiments, Ab is an anti-Her2 monoclonal antibody, such as Trastuzumab, Pertuzumab.

In some embodiments, Ab is an antibody or antigen-binding fragment thereof. In some embodiments, Ab is an antibody or antigen-binding fragment thereof that specifically binds to epidermal growth factor receptor 2 (Her2), a member of the ErbB family of receptor tyrosine kinases.

In some embodiments, the antibody or antigen-binding fragment thereof comprises:
(1) the following heavy chain variable region (VH) and/or light chain variable region (VL):
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL):
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL):
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof,
wherein, the variant described in any one of (3a), and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL):
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following three CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof, or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some preferred embodiments, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof,
wherein, the variant described in any one of (3a), and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable regions (VH) and/or light chain variable regions (VL), wherein the CDRs are defined according to the IMGT numbering system:
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following three CDRs: a CDR-L1 having a sequence as set forth in SEQ ID NO: 16 or variant thereof, and a CDR-L2 having a sequence as set forth in SEQ ID NO: 17 or variant thereof, and a CDR-L3 having a sequence as set forth in SEQ ID NO: 10 or variant thereof, or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof,
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some embodiments, the antibody or antigen-binding fragment thereof comprises:
(1) the following heavy chain variable region (VH) and light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25.

In some embodiments, the antibody or antigen-binding fragment thereof comprises:
(a) a VH as set forth in SEQ ID NO: 1 or variant thereof, and/or, a VL as set forth in SEQ ID NO: 2 or variant thereof; or
(b) a VH as set forth in SEQ ID NO: 3 or variant thereof, and/or, a VL as set forth in SEQ ID NO: 4 or variant thereof; or
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution.

In some embodiments, the antibody or antigen-binding fragment thereof comprises:
(a) a VH as set forth in SEQ ID NO: 1, and, a VL as set forth in SEQ ID NO: 2; or
(b) a VH as set forth in SEQ ID NO: 3, and, a VL as set forth in SEQ ID NO: 4.

In some embodiments, the antibody or antigen-binding fragment thereof further comprises:
(a) a heavy chain constant region (CH) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids); and
(b) a light chain constant region (CL) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids).

In some embodiments, the heavy chain constant region is an IgG heavy chain constant region, such as an IgG1, IgG2, IgG3 or IgG4 heavy chain constant region, such as a human IgG1 heavy chain constant region or a human IgG4 heavy chain constant region.

In some embodiments, the antibody or antigen-binding fragment thereof comprises a heavy chain constant region (CH) as set forth in SEQ ID NO: 35 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 35.

In some embodiments, the antibody or antigen-binding fragment thereof comprises a light chain constant region (CL) as set forth in SEQ ID NO: 36 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 36.

In some embodiments, the antibody or antigen-binding fragment thereof comprises a heavy chain constant region (CH) as set forth in SEQ ID NO: 35 and a light chain constant region (CL) as set forth in SEQ ID NO: 36.

In some embodiments, the antibody or antigen-binding fragment thereof comprises:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36.

In some embodiments, the antibody or antigen-binding fragment thereof comprises:
(1) a heavy chain comprising the sequence as set forth in SEQ ID NO: 37, and, a light chain comprising the sequence as set forth in SEQ ID NO: 38; or
(2) a heavy chain comprising the sequence as set forth in SEQ ID NO: 39, and, a light chain comprising the sequence as set forth in SEQ ID NO: 40.

In some embodiments, the antibody or antigen-binding fragment thereof is selected from Trastuzumab or Pertuzumab. The query accession number (IMGT/mAb-DB ID) for the amino acid sequence of Trastuzumab in the IMGT database is: 97. The query accession number (IMGT/mAb-DB ID) for the amino acid sequence of Pertuzumab in the IMGT database is: 80.

In certain embodiments, the antibody or antigen-binding fragment disclosed herein has a heavy chain constant region that may comprise a C-terminal lysine lack a C-terminal lysine, or lack a C-terminal glycine-lysine dipeptide. In some embodiments, the N-terminal amino acid of the antibody or antigen-binding fragment thereof may be cyclized to pyroglutamic acid.

As known to those skilled in the art, pyroglutamic acid is the conjugate acid of pyroglutamate and is in equilibrium with pyroglutamate in solution.

In certain embodiments, provided herein is a composition comprising the antibody or antigen-binding fragments disclosed herein, wherein each antibody or antigen-binding fragment thereof may independently comprise a C-terminal lysine, lack a C-terminal lysine, lack a C-terminal glycine-lysine and/or comprise a N-terminal glutamine or glutamic acid, or a N-terminal amino acid cyclized to pyroglutamic acid or pyroglutamate.

In certain embodiments, the antibody or antigen-binding fragment disclosed herein comprises those capable of specifically binding to an antigen and may comprise post-translational modifications thereof (e.g., cleavage of the C-terminal lysine in the heavy chain, conversion of the N-terminal glutamine or glutamic acid in the heavy or light chain to pyroglutamic acid or pyroglutamate), which may occur during recombinant expression in a host cell (e.g., CHO cell) or arise during purification/storage.

In certain embodiments, the N-terminal glutamine of the VH having the sequence as set forth in SEQ ID NO: 1 or 3 or variant thereof or the heavy chain having the sequence as set forth in SEQ ID NO: 37 or 39 or variant thereof undergoes cyclization to form pyroglutamic acid or pyroglutamate.

In certain embodiments, the heavy chain constant region (CH) as set forth in SEQ ID NO: 35 or variant thereof or the heavy chain having the sequence as set forth in SEQ ID NO: 37 or 39 or variant thereof lacks C-terminal lysine.

In some embodiments, M is linked to a sulfhydryl group (—SH) on Ab.

In some embodiments, in an antibody-drug conjugate having a structure represented by Ab-[M-L-E-D]ₓ, x is 1 to 10, and M-L-E-D is formed from a compound represented by D-E-L-M′, preferably by removing Lg from D-E-L-M′, wherein the compound represented by D-E-L-M′ is as defined above, and Ab is as defined above.

In some embodiments, in the antibody-drug conjugate having the structure represented by Ab-[M-L-E-D]x, x is 1 to 10, M-L-E-D is formed from the compounds shown in A-1 to A-204 or B-1 to B-24, preferably by removing —SO₂Me from the compounds, and Ab is as defined above.

In some embodiments, the antibody-drug conjugate is selected from the group consisting of ADC A-1 to ADC A-204, ADC B-1 to ADC B-24 shown below

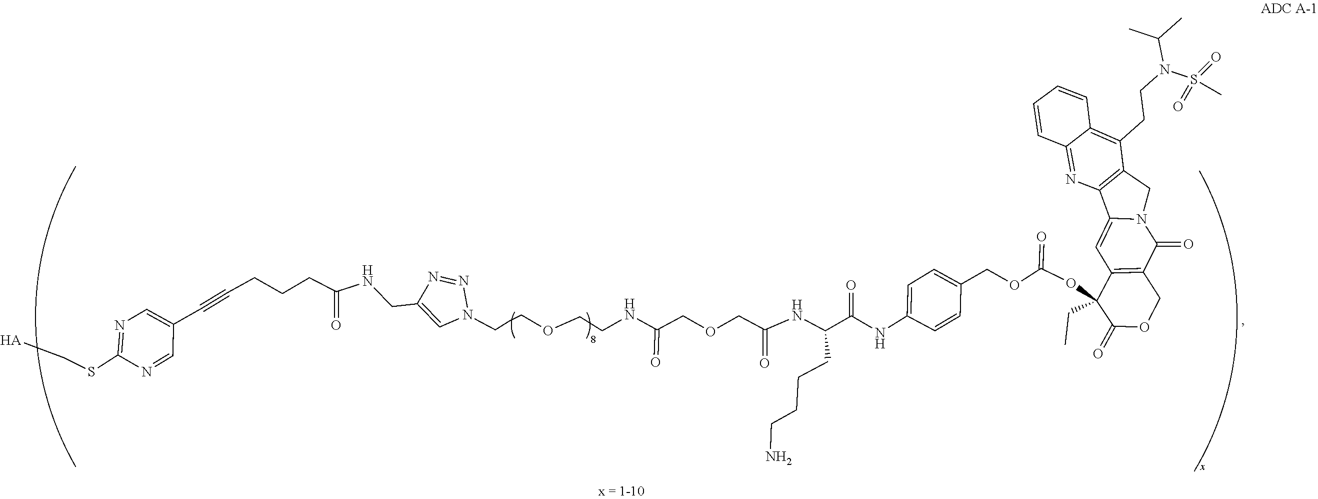

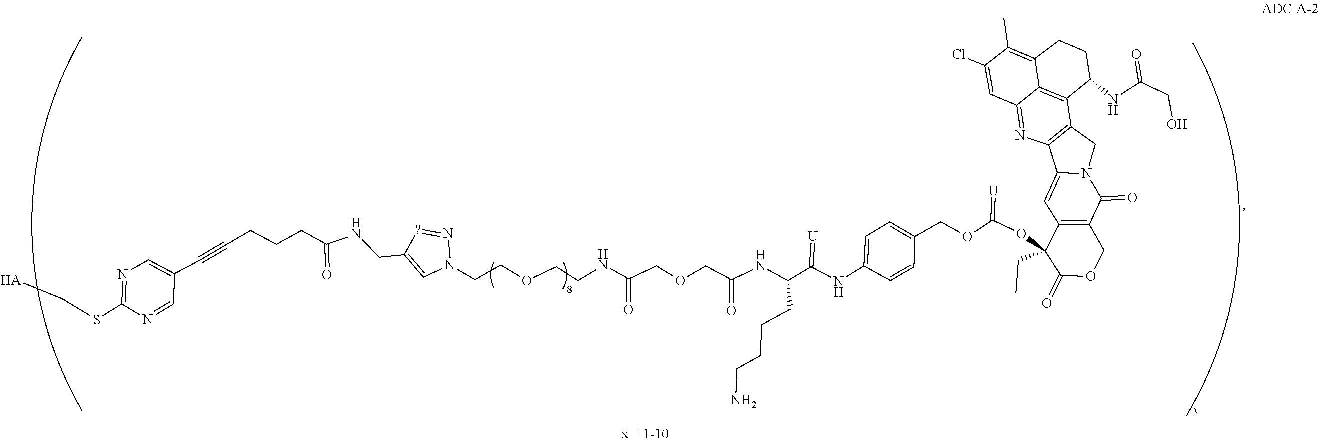

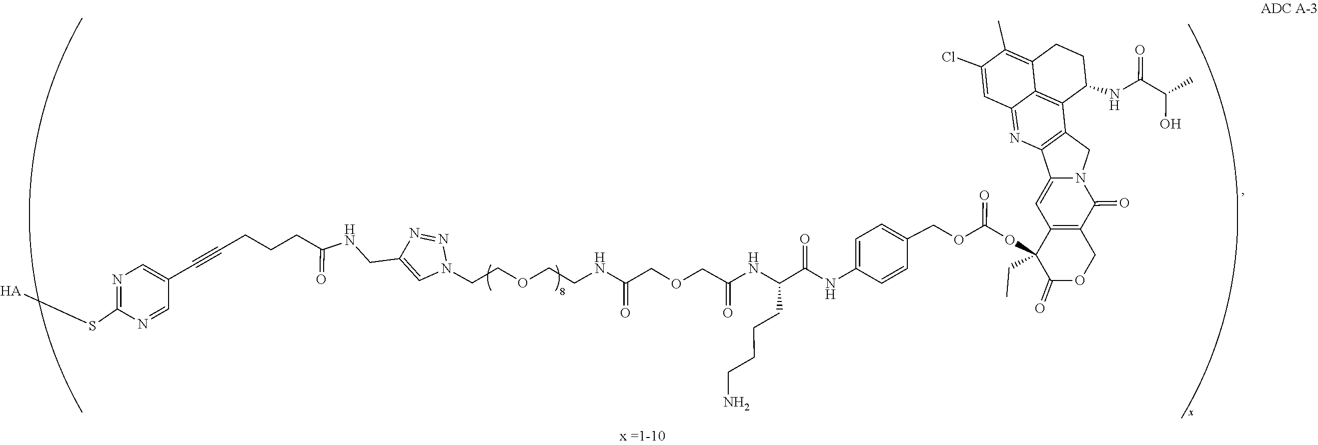

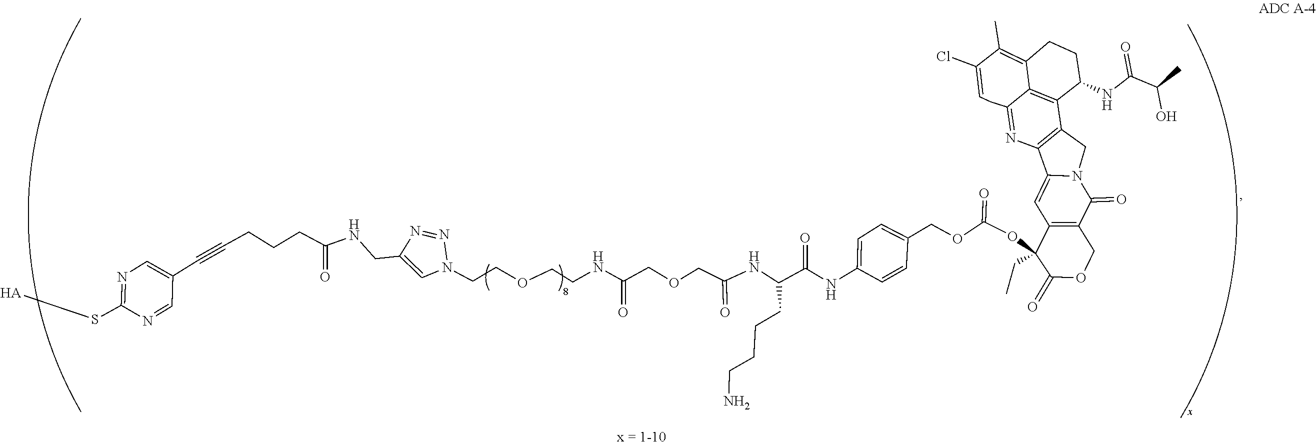

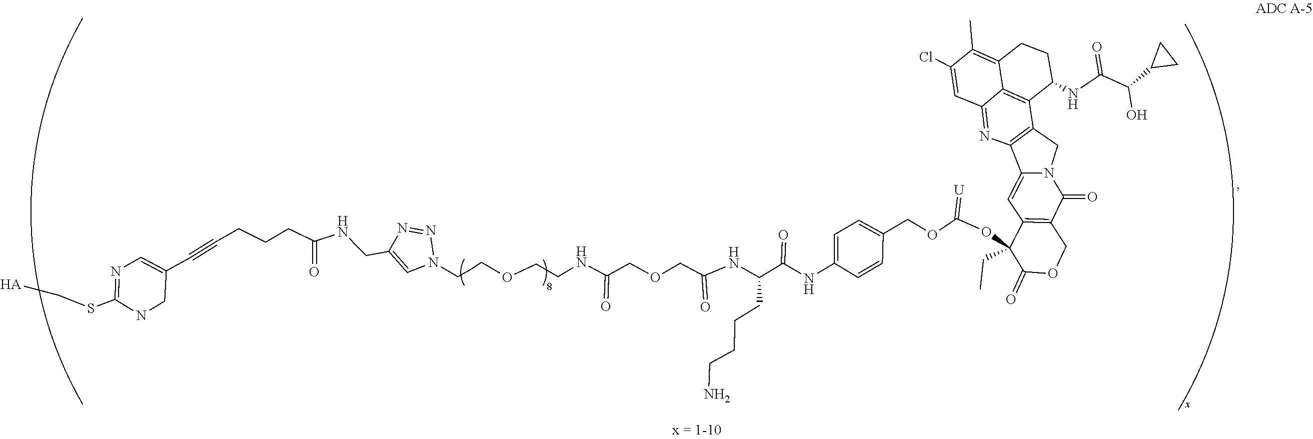

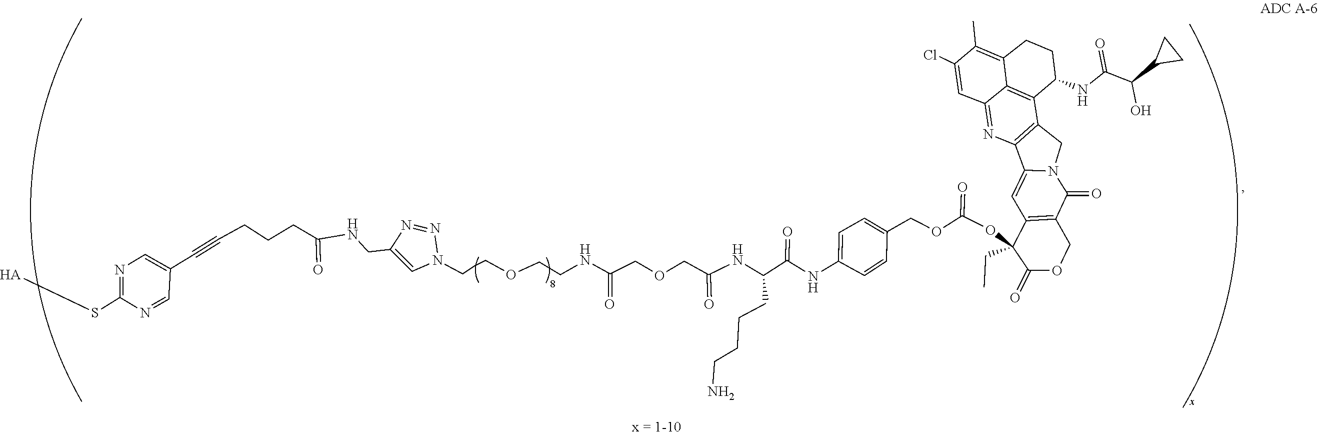

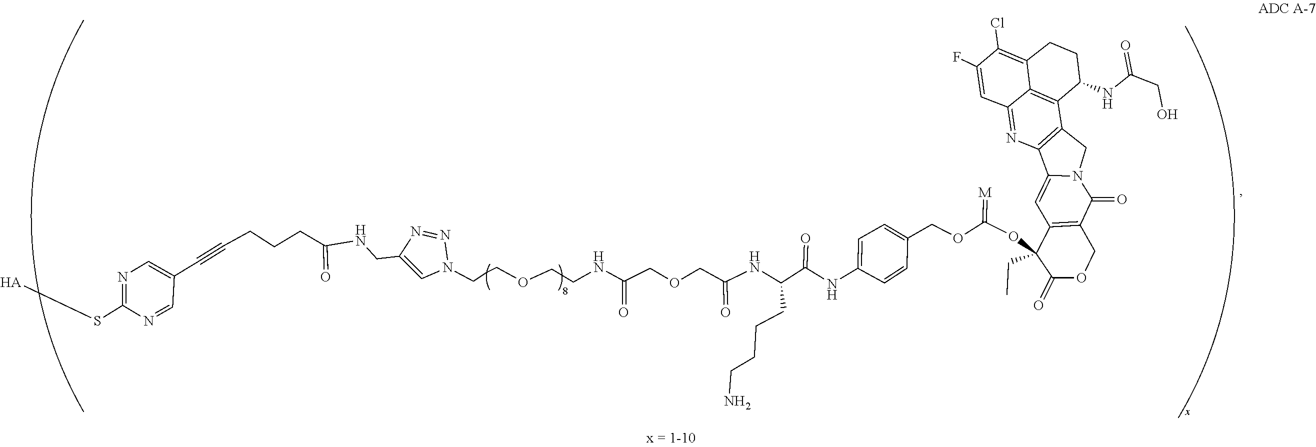

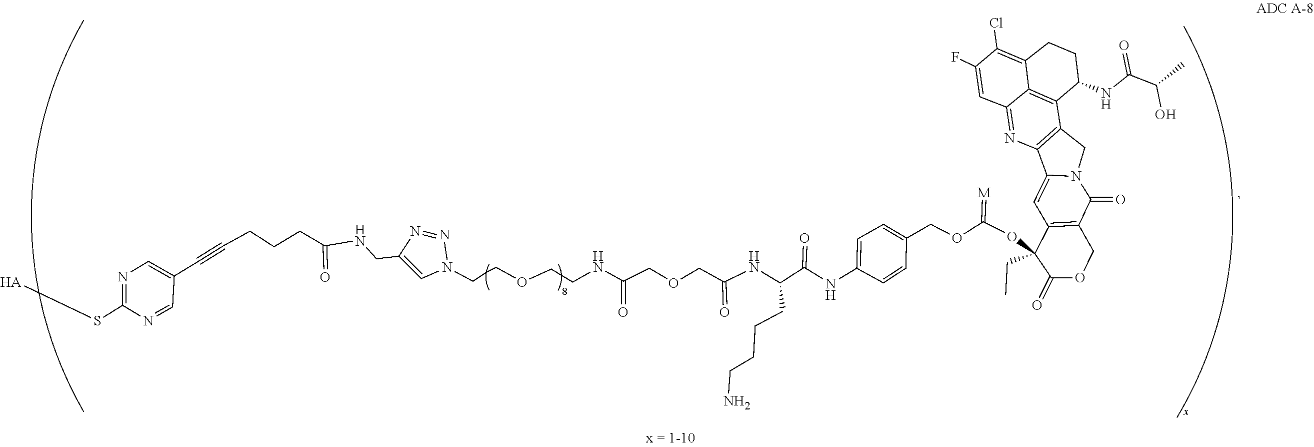

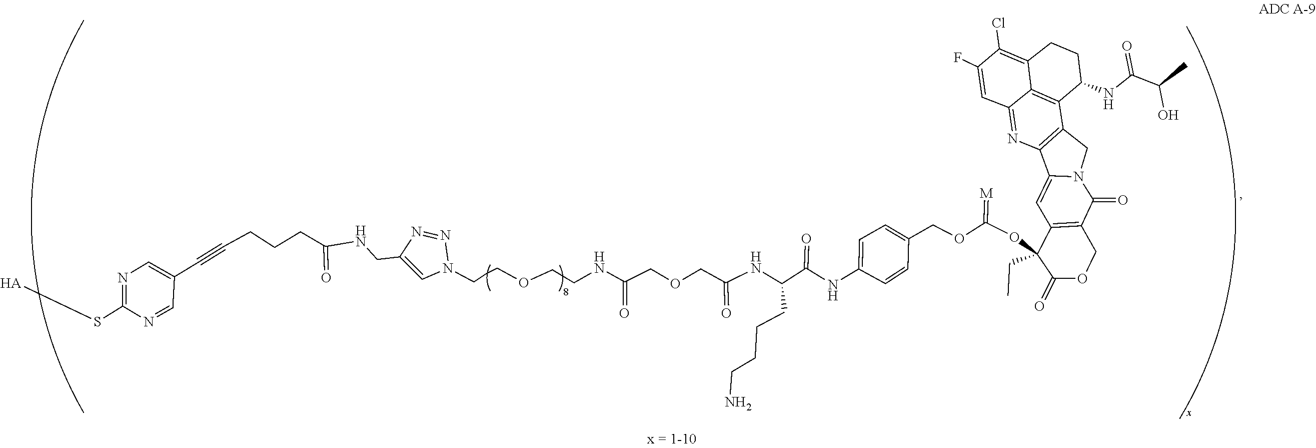

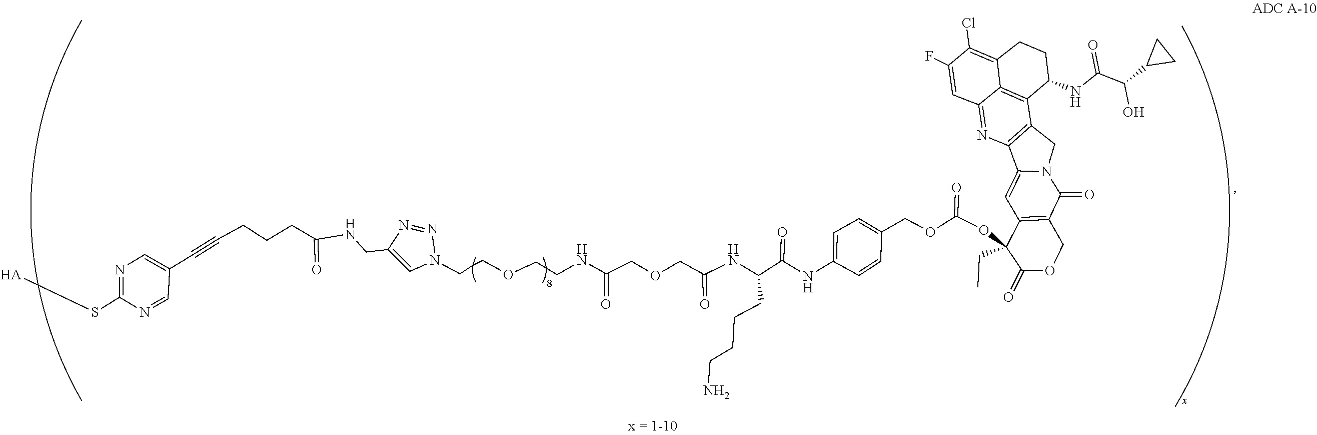

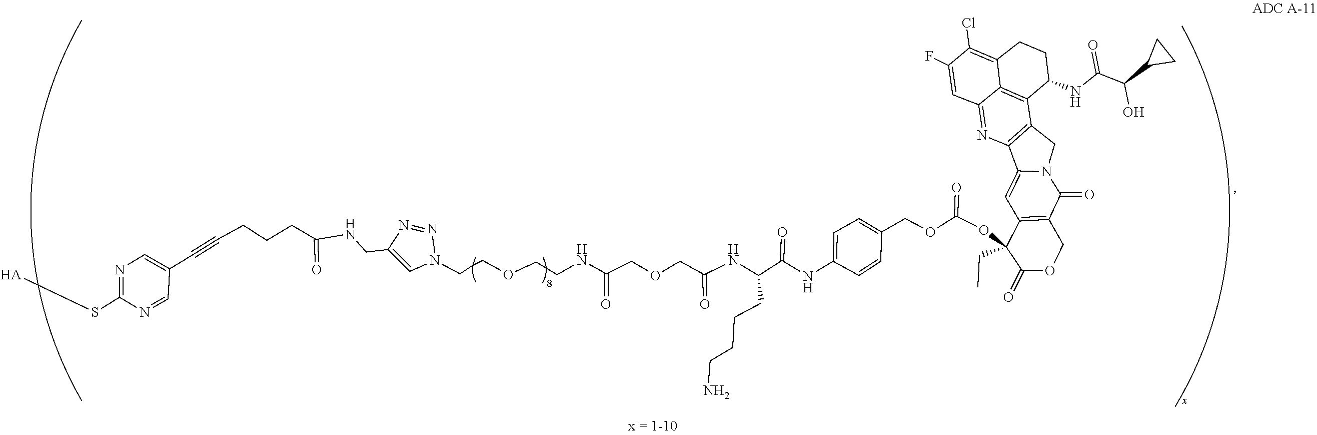

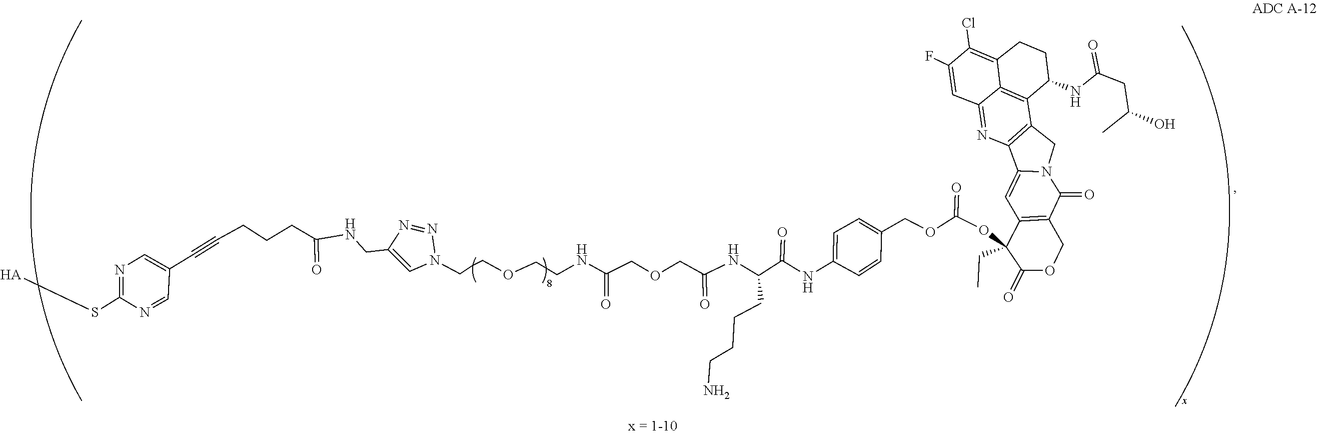

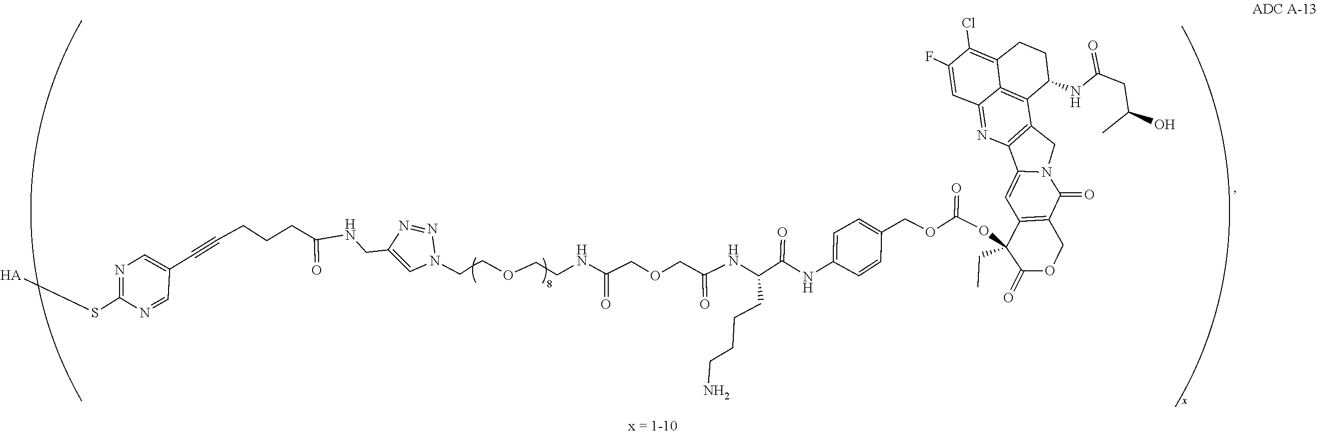

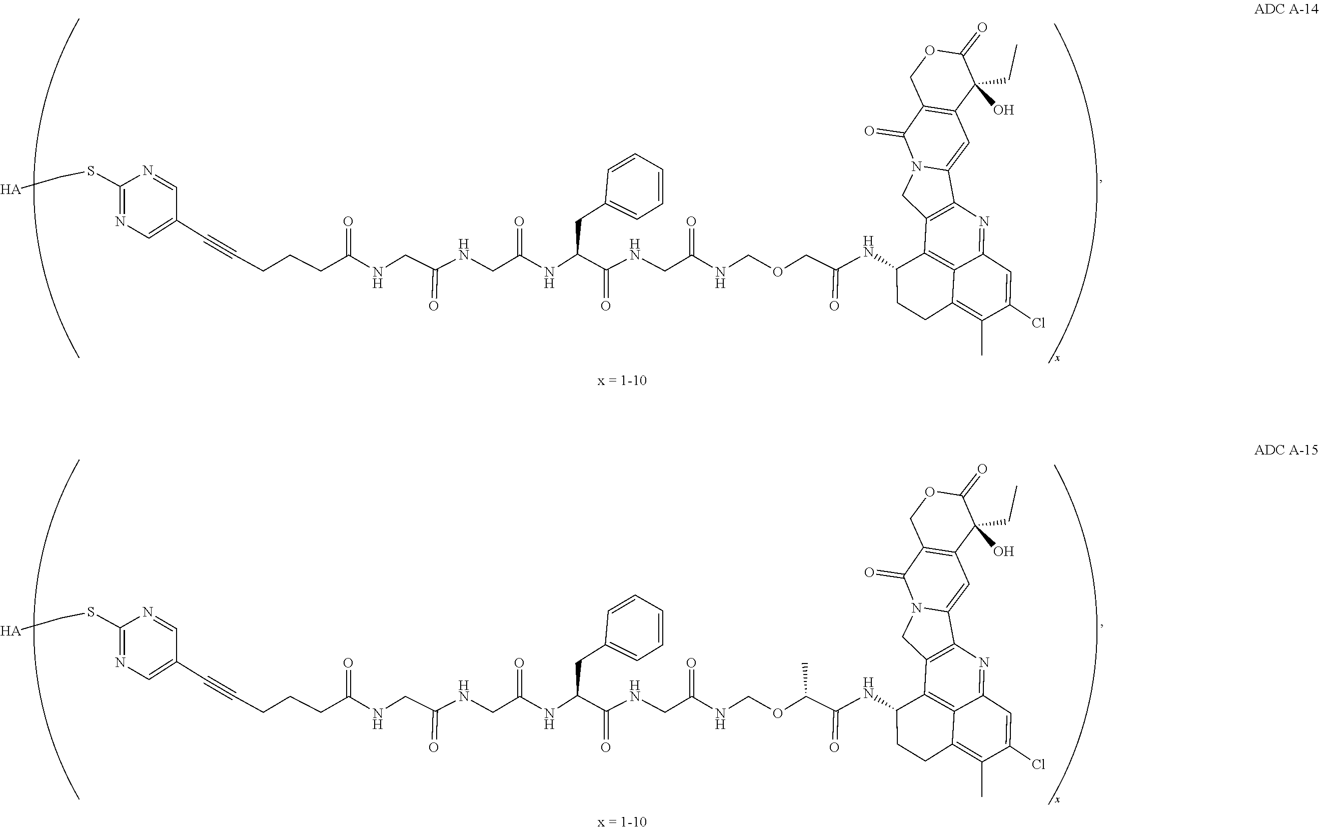

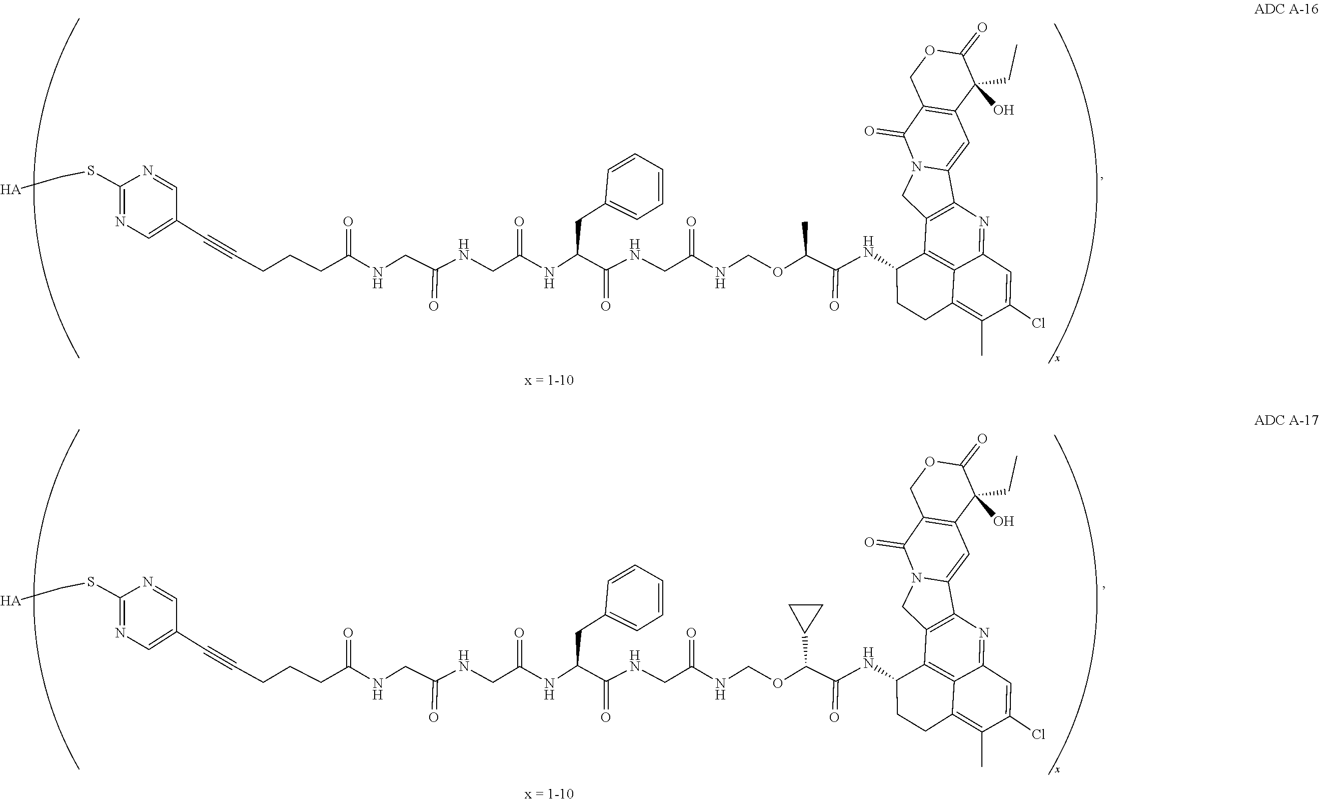

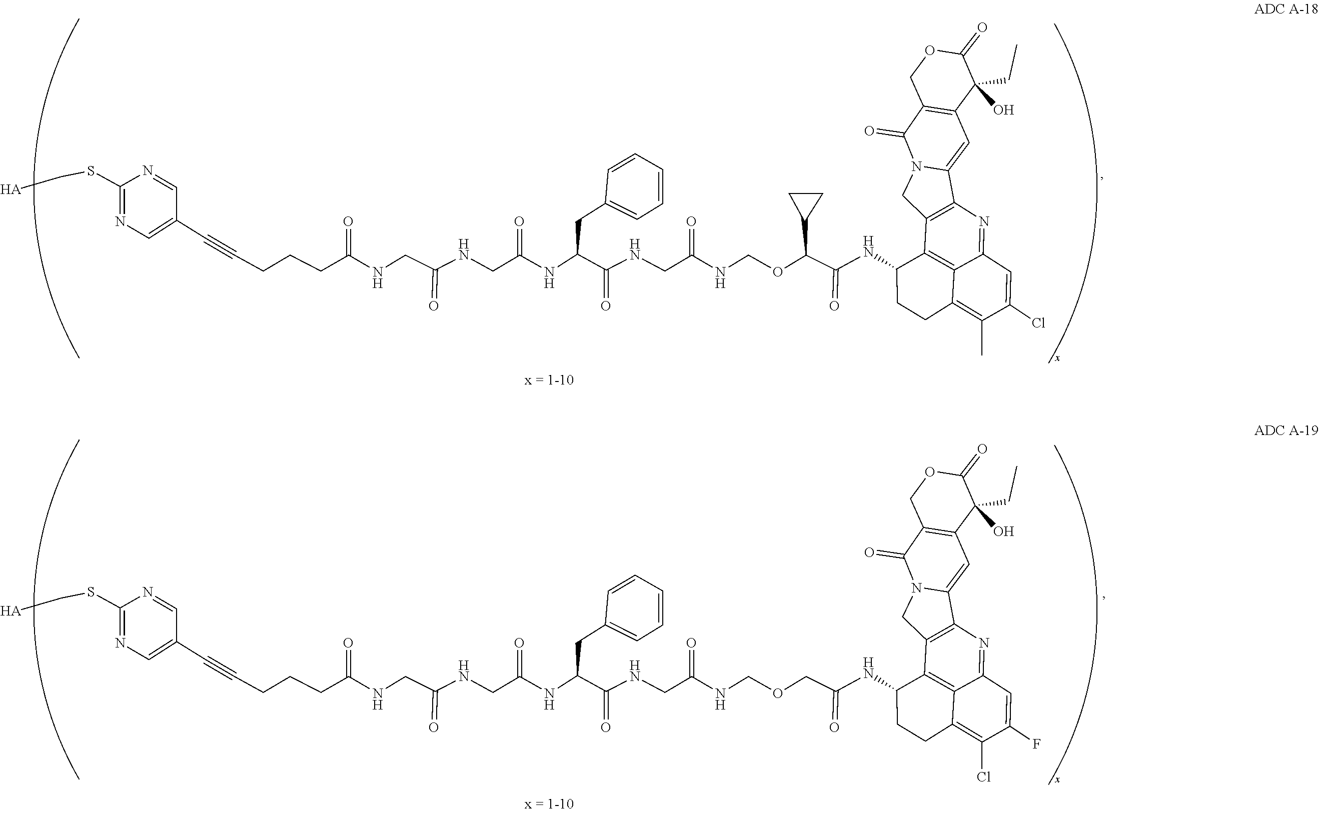

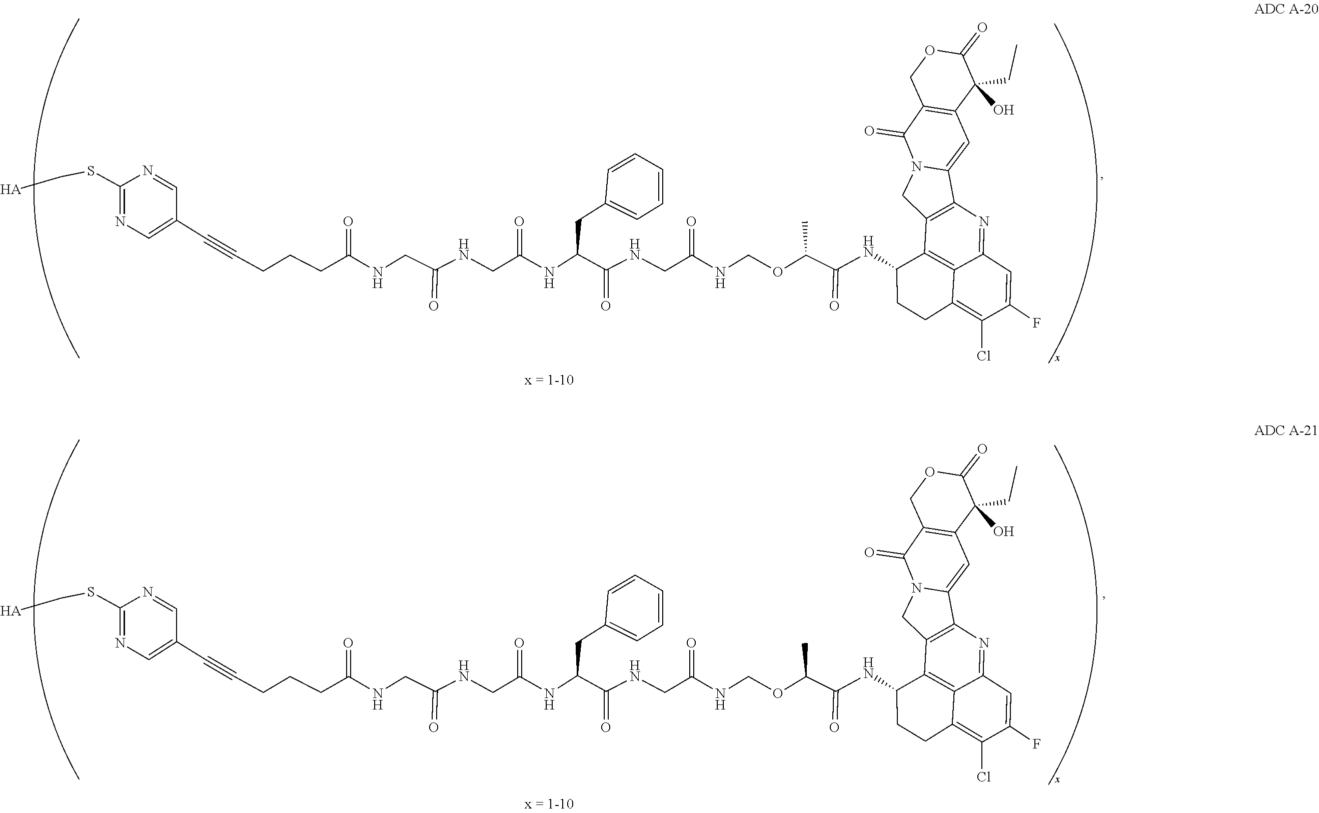

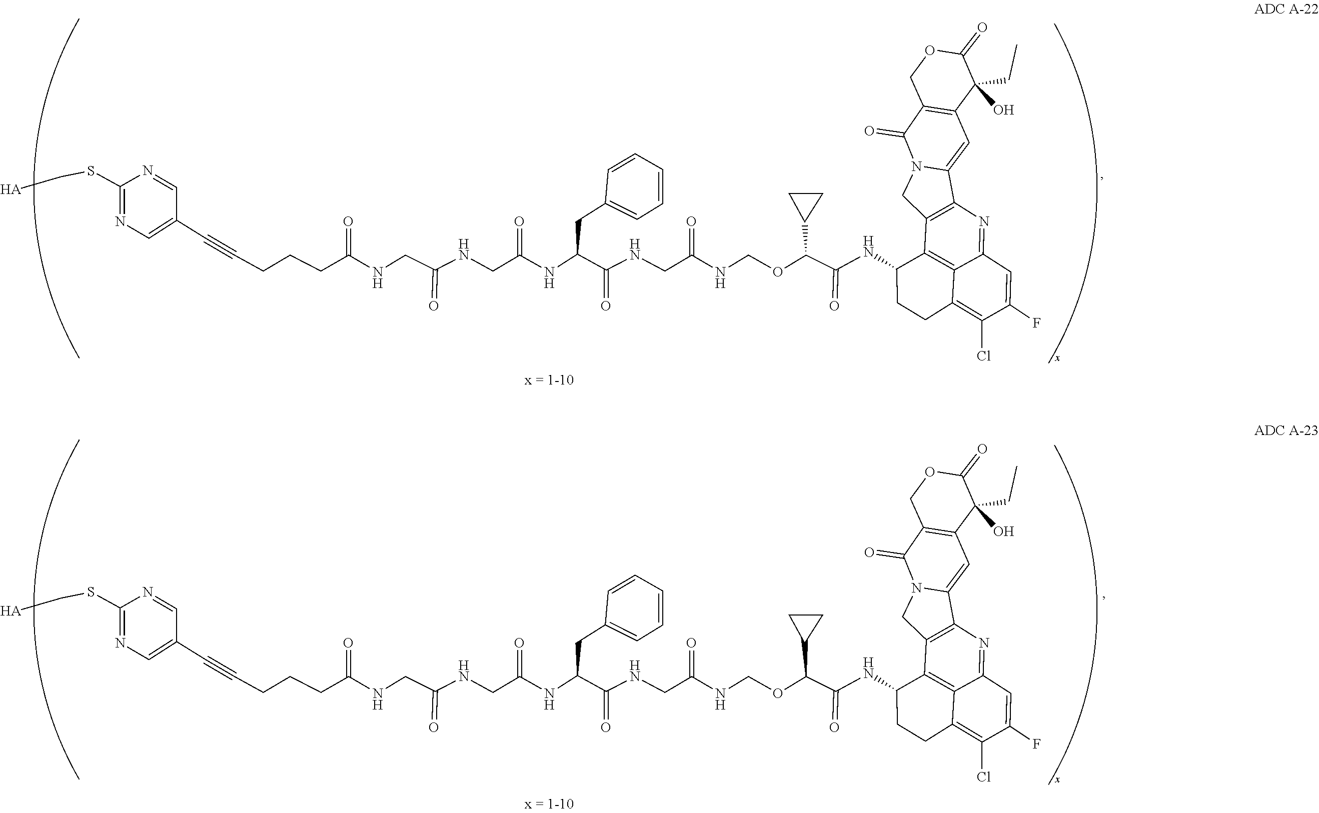

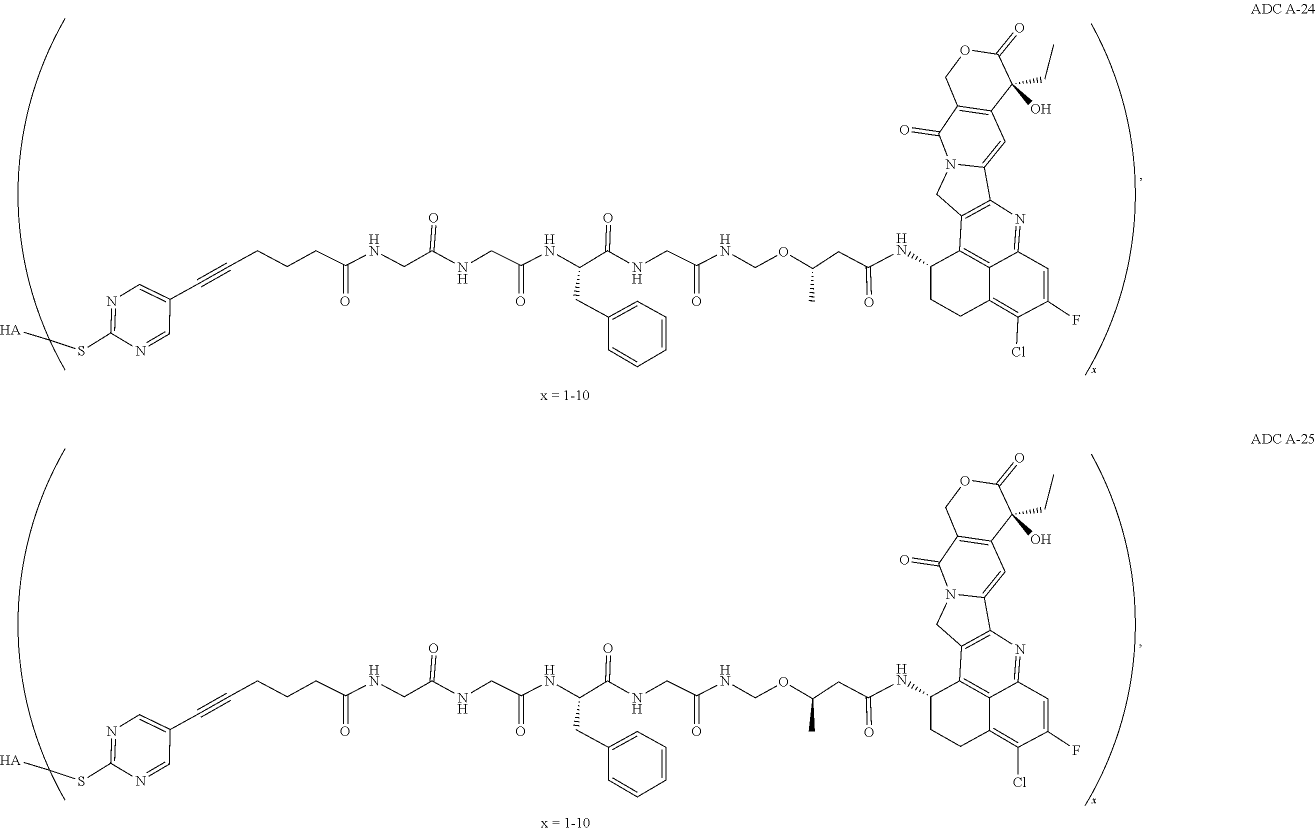

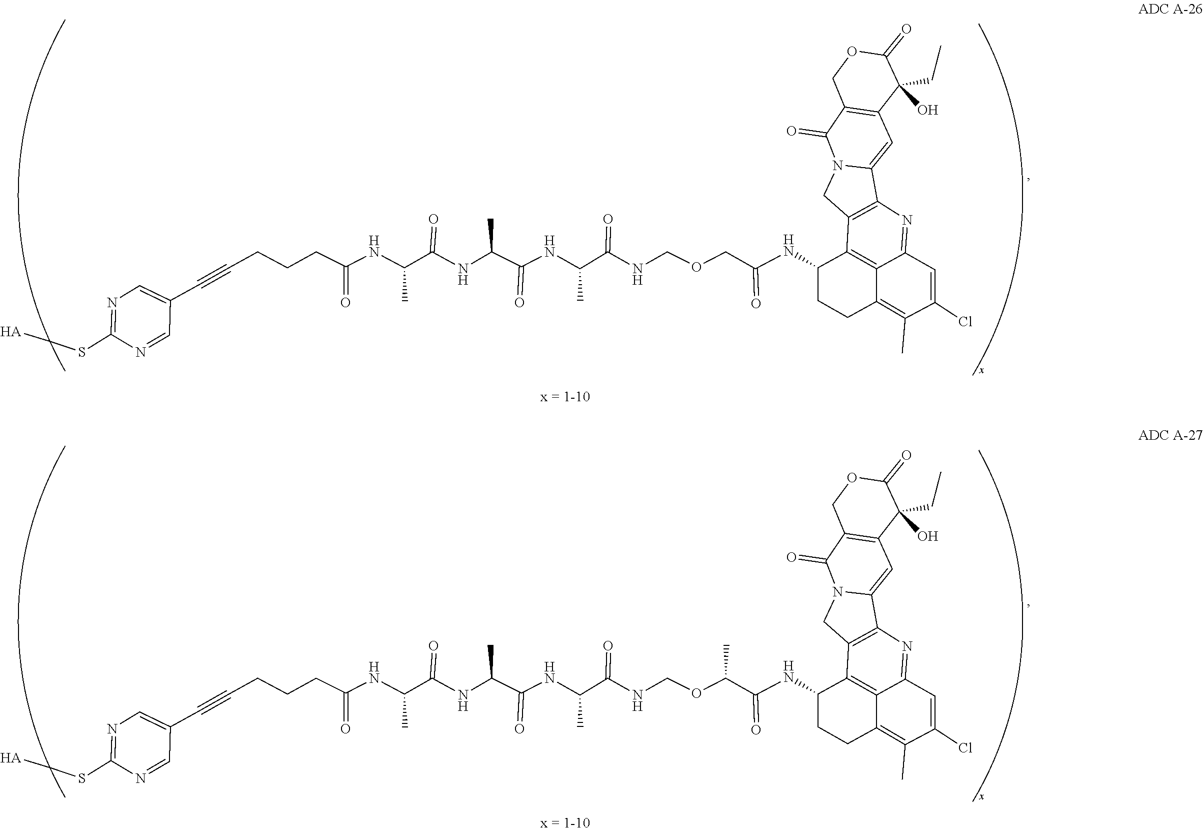

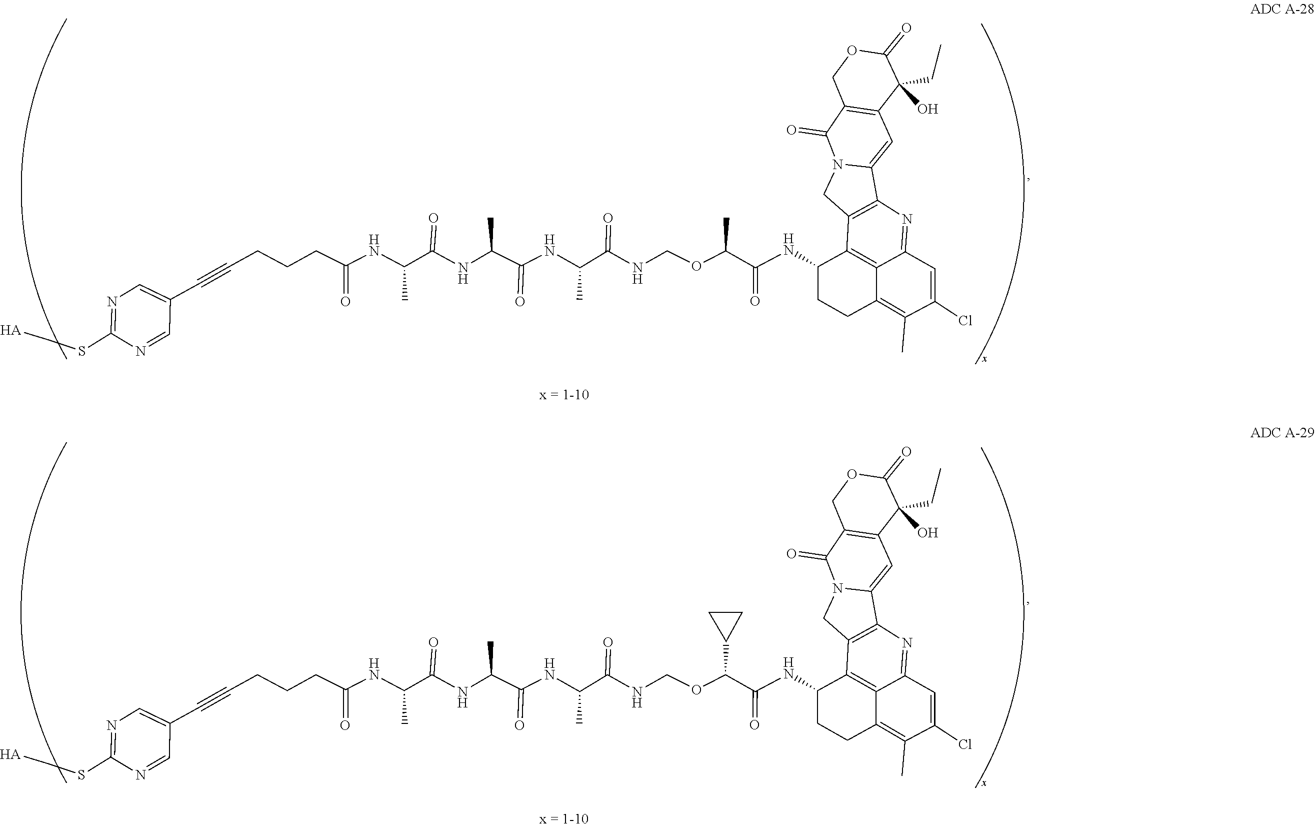

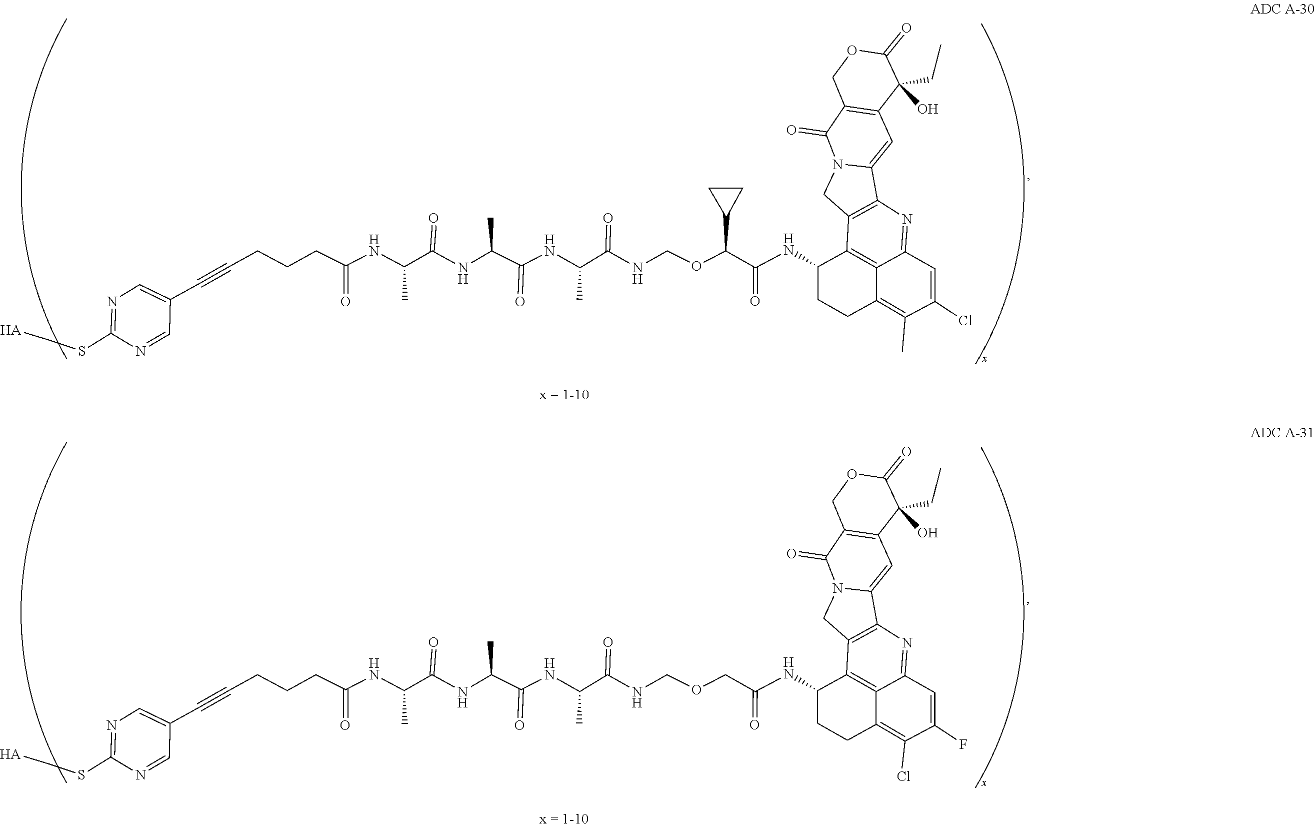

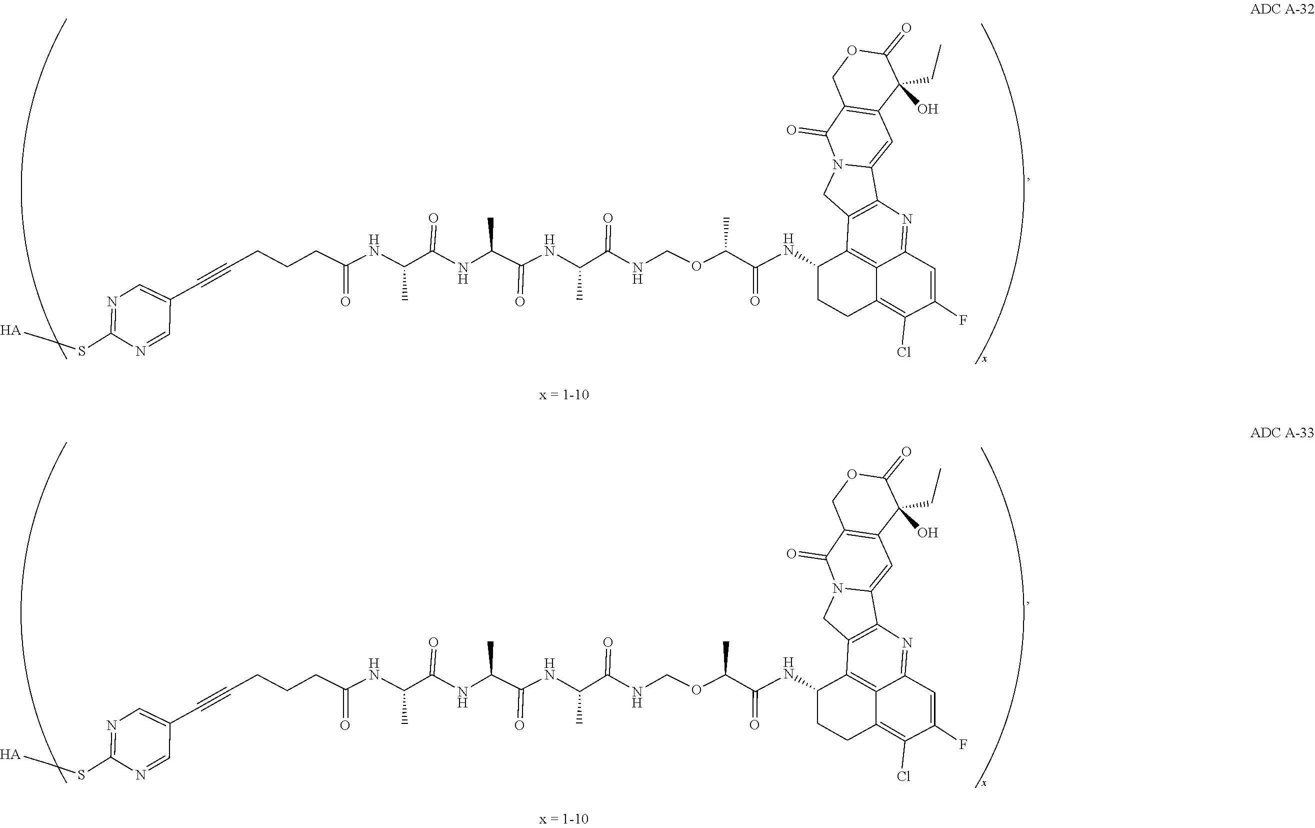

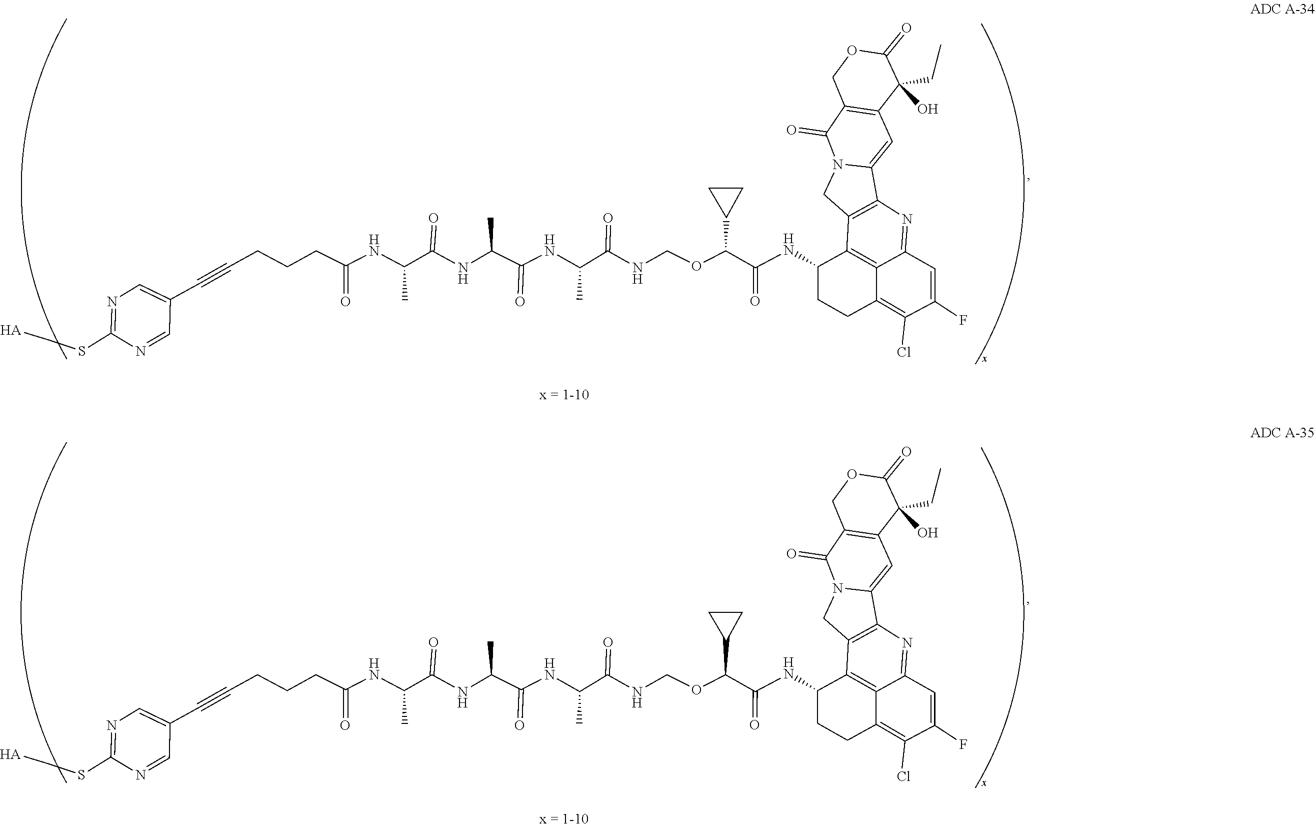

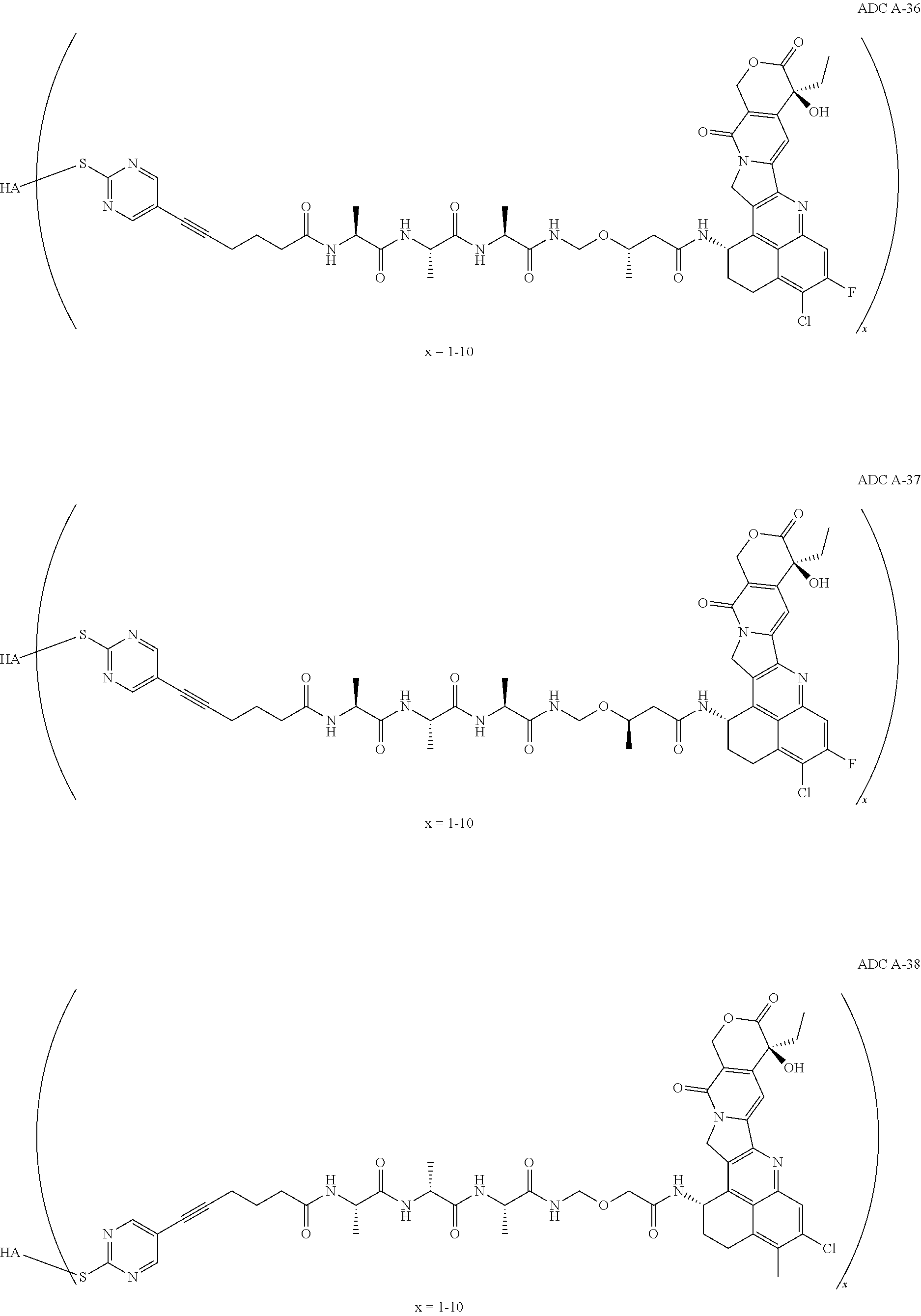

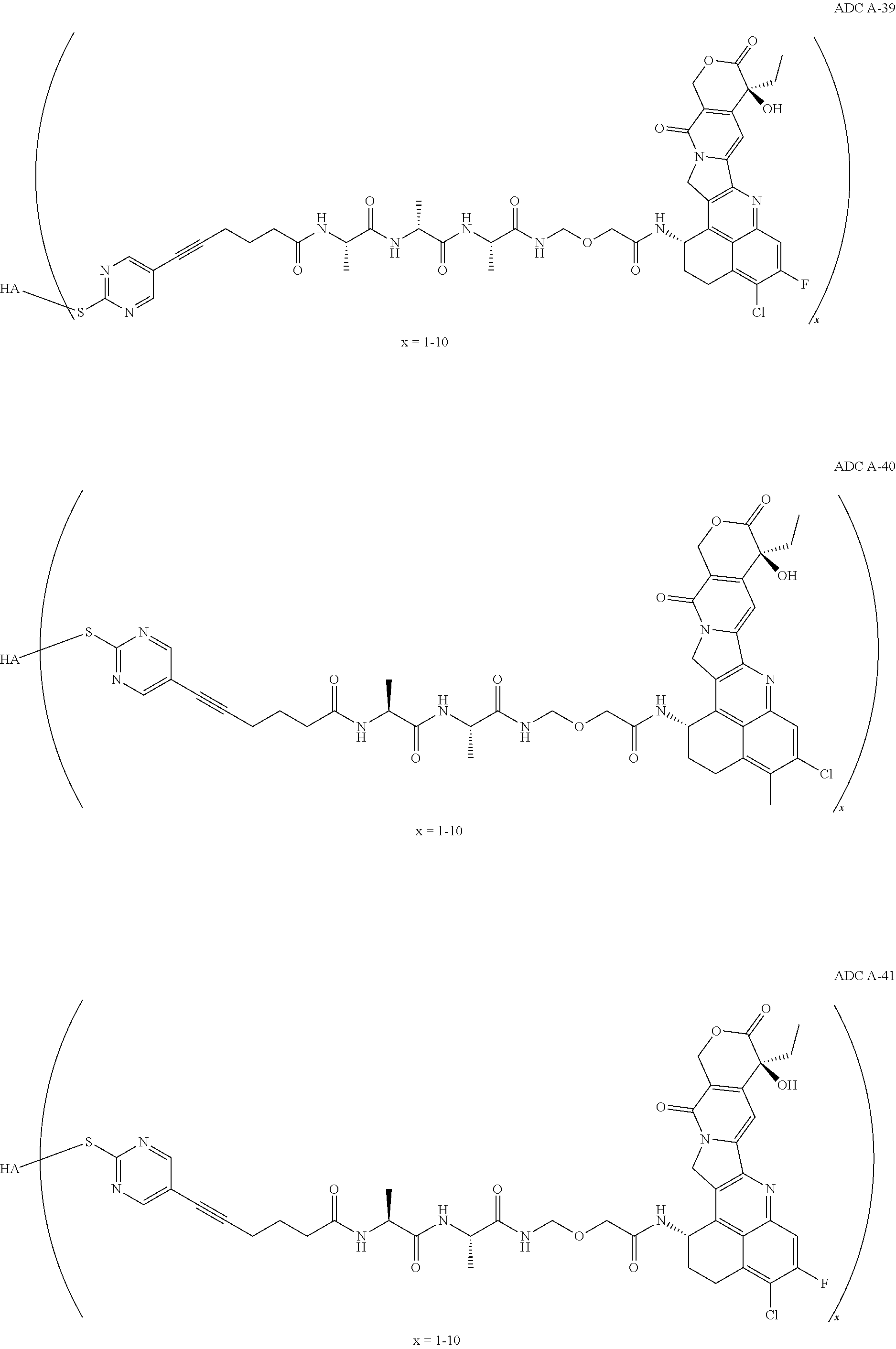

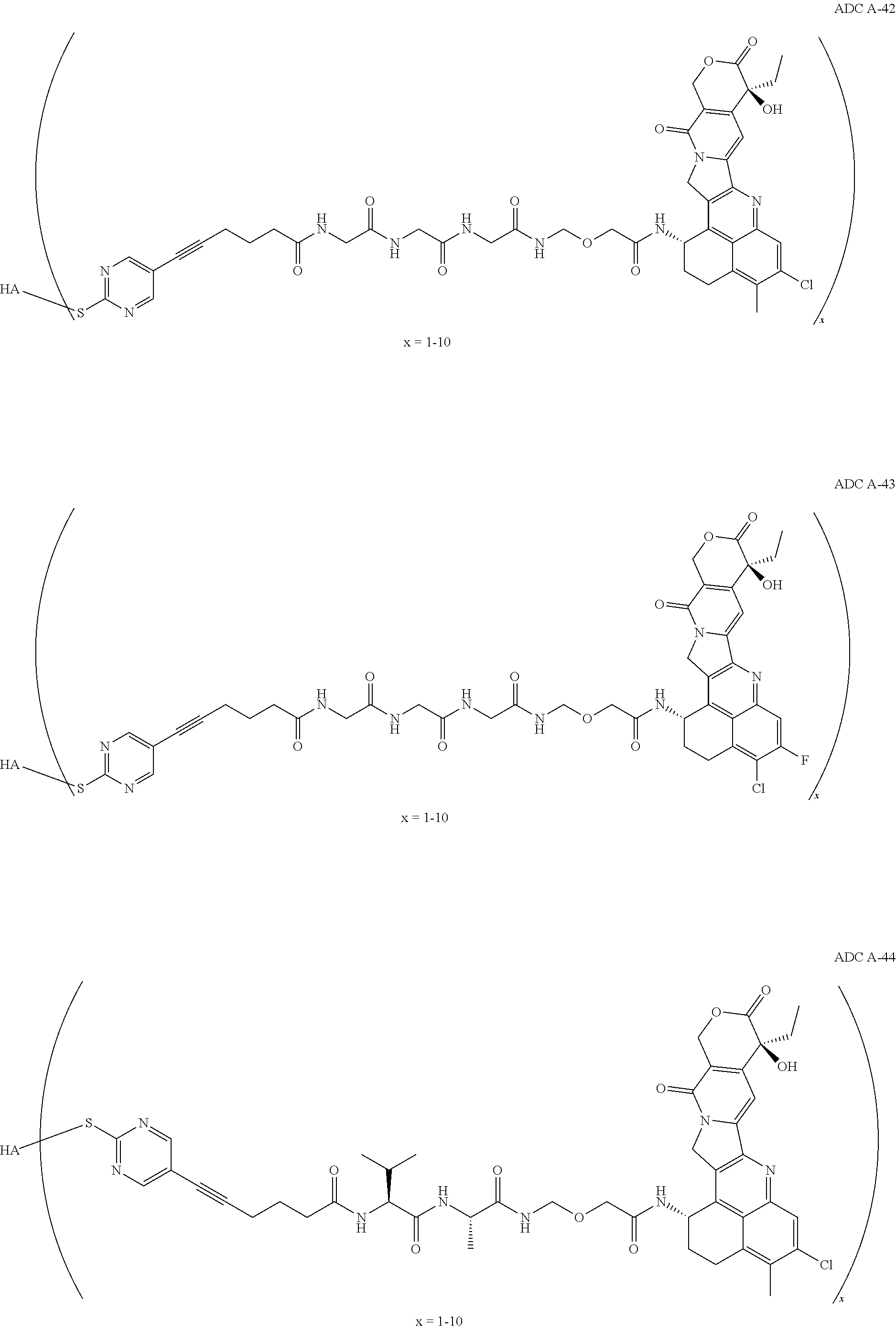

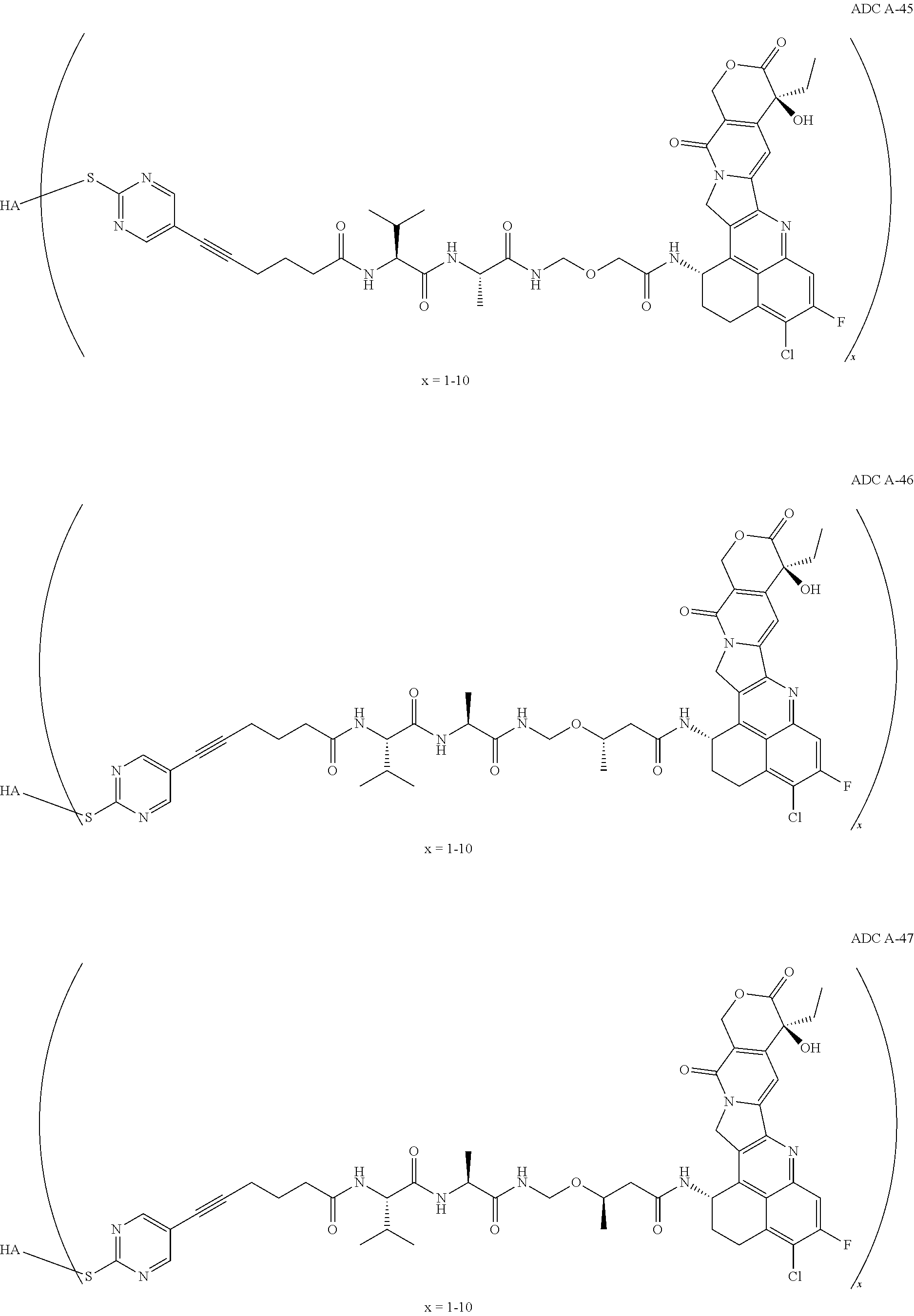

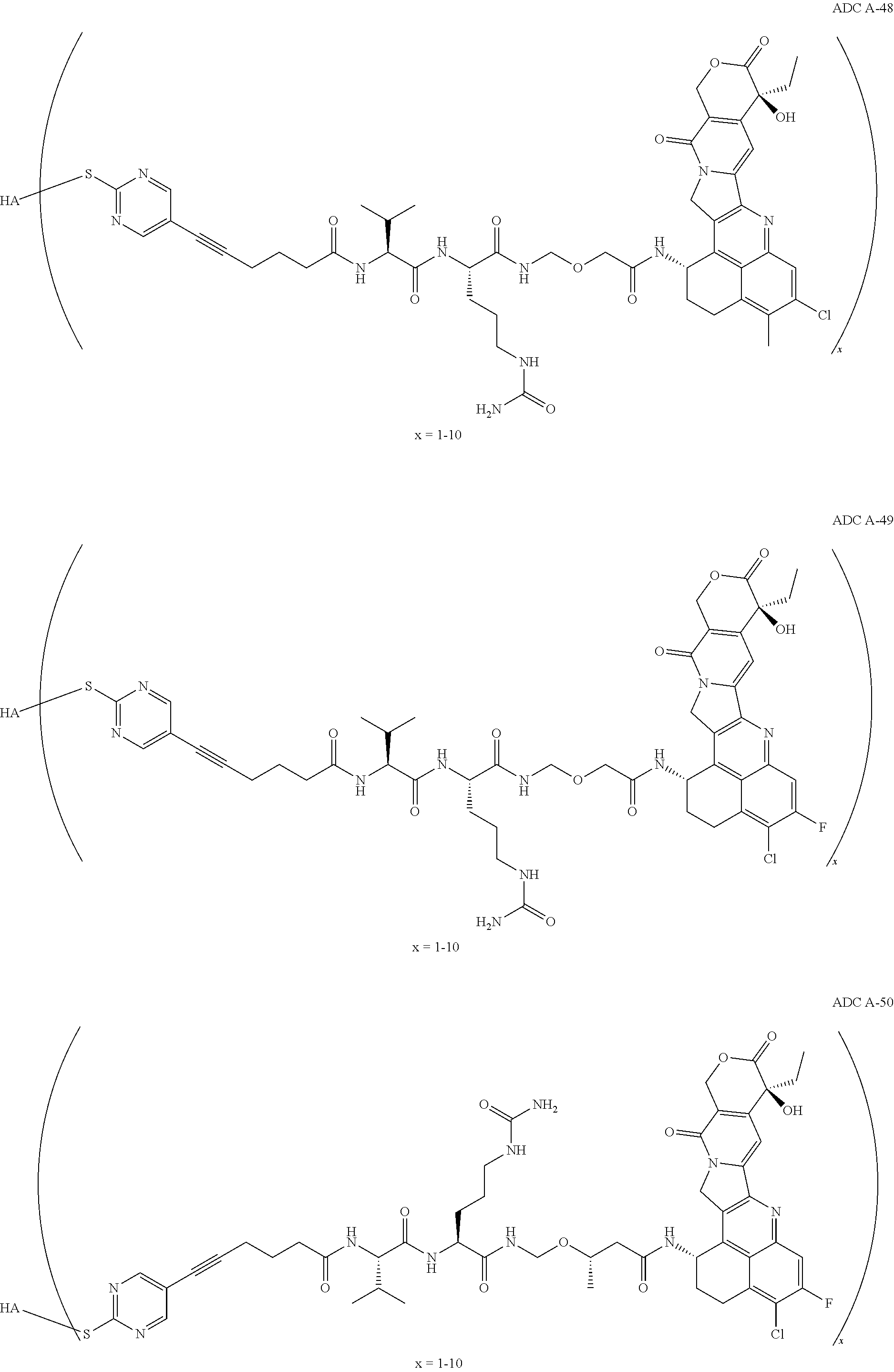

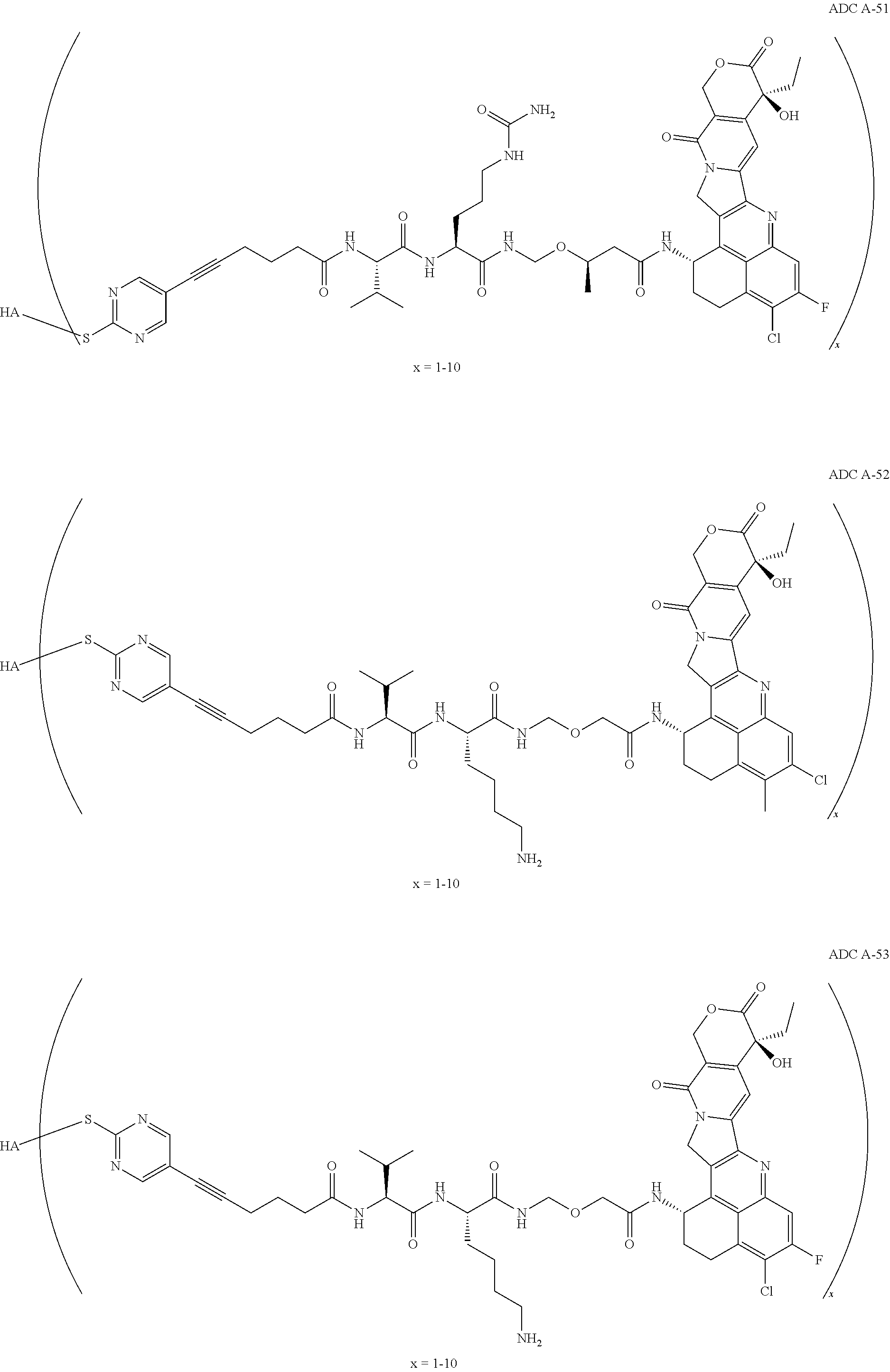

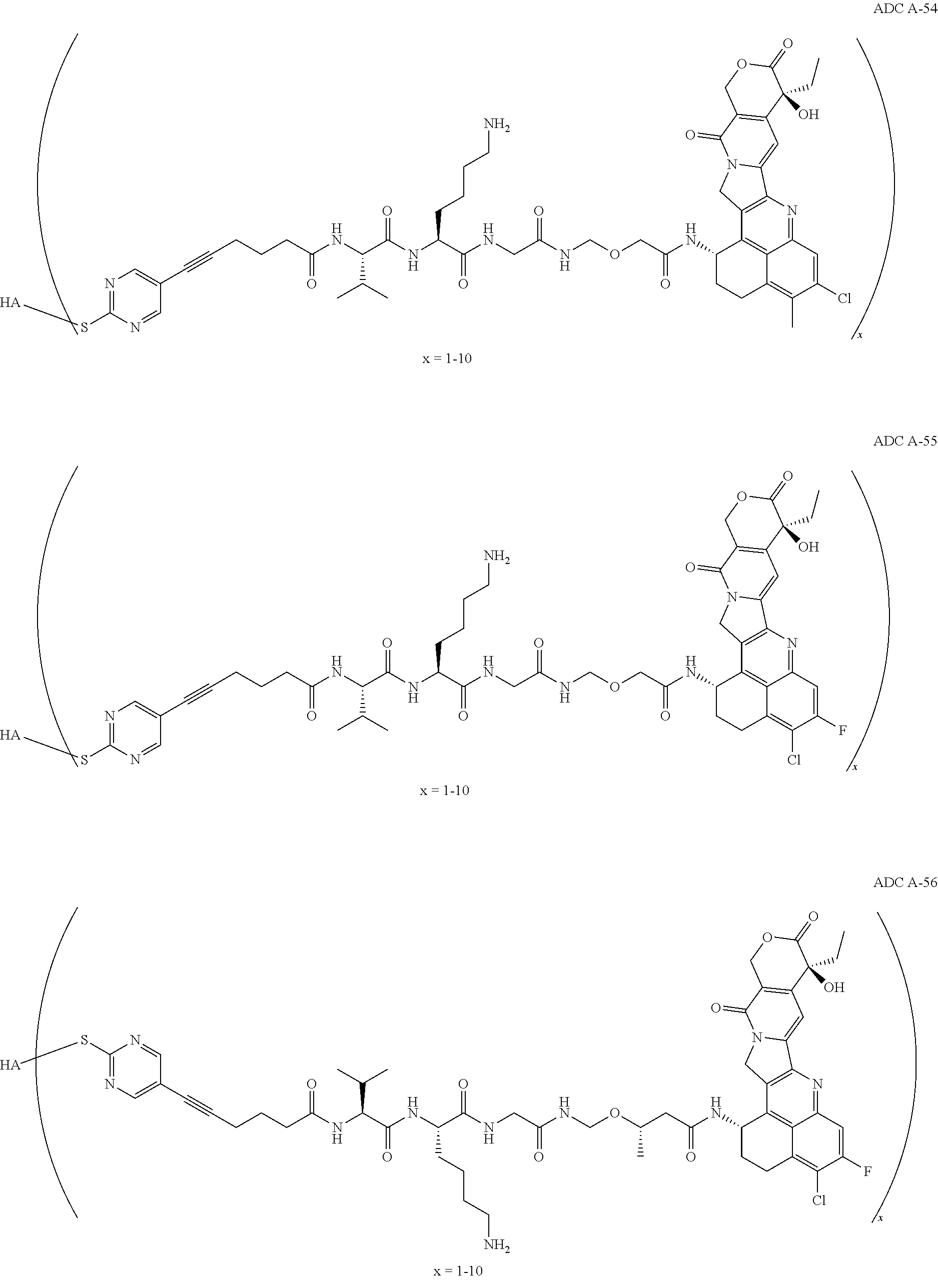

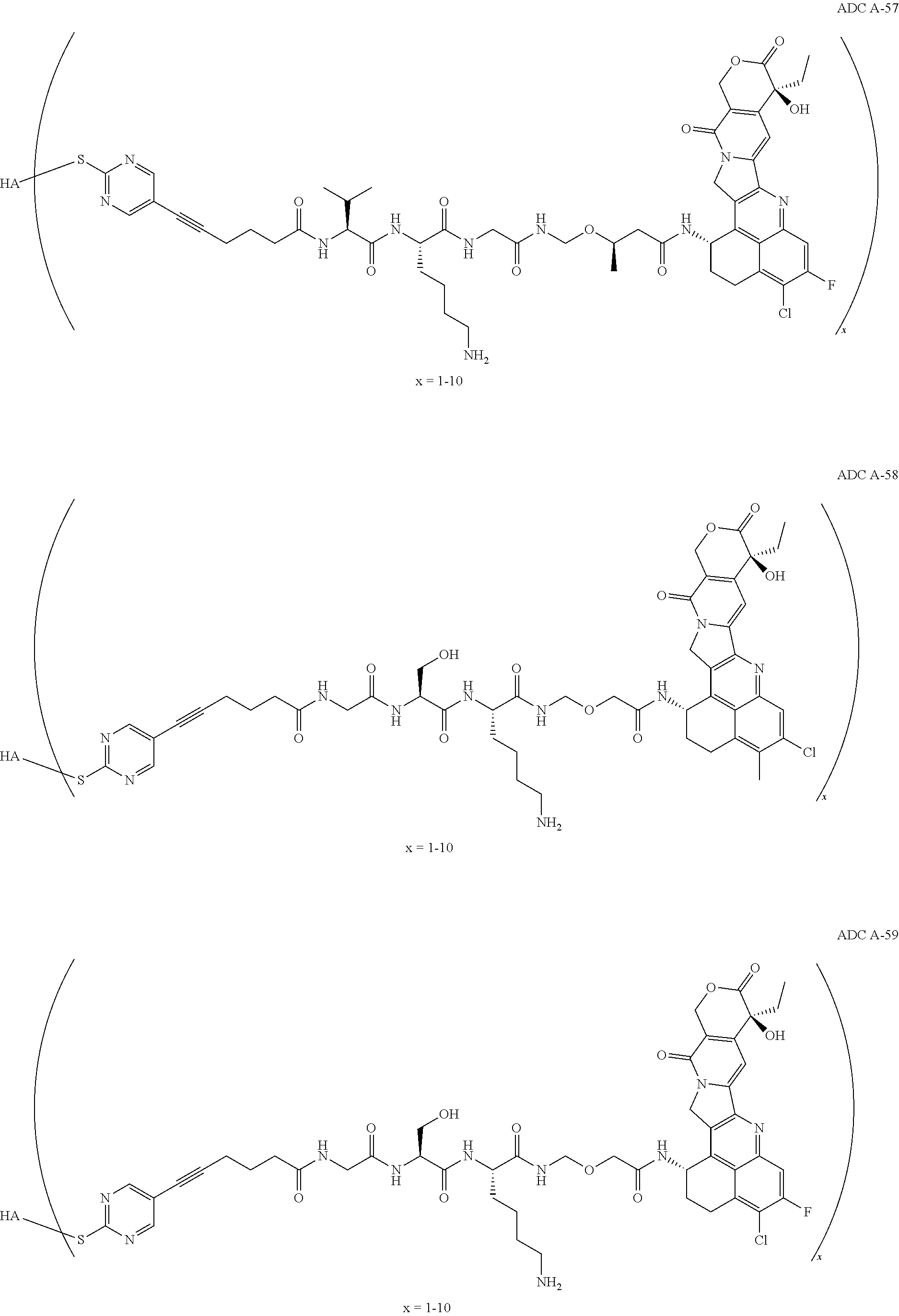

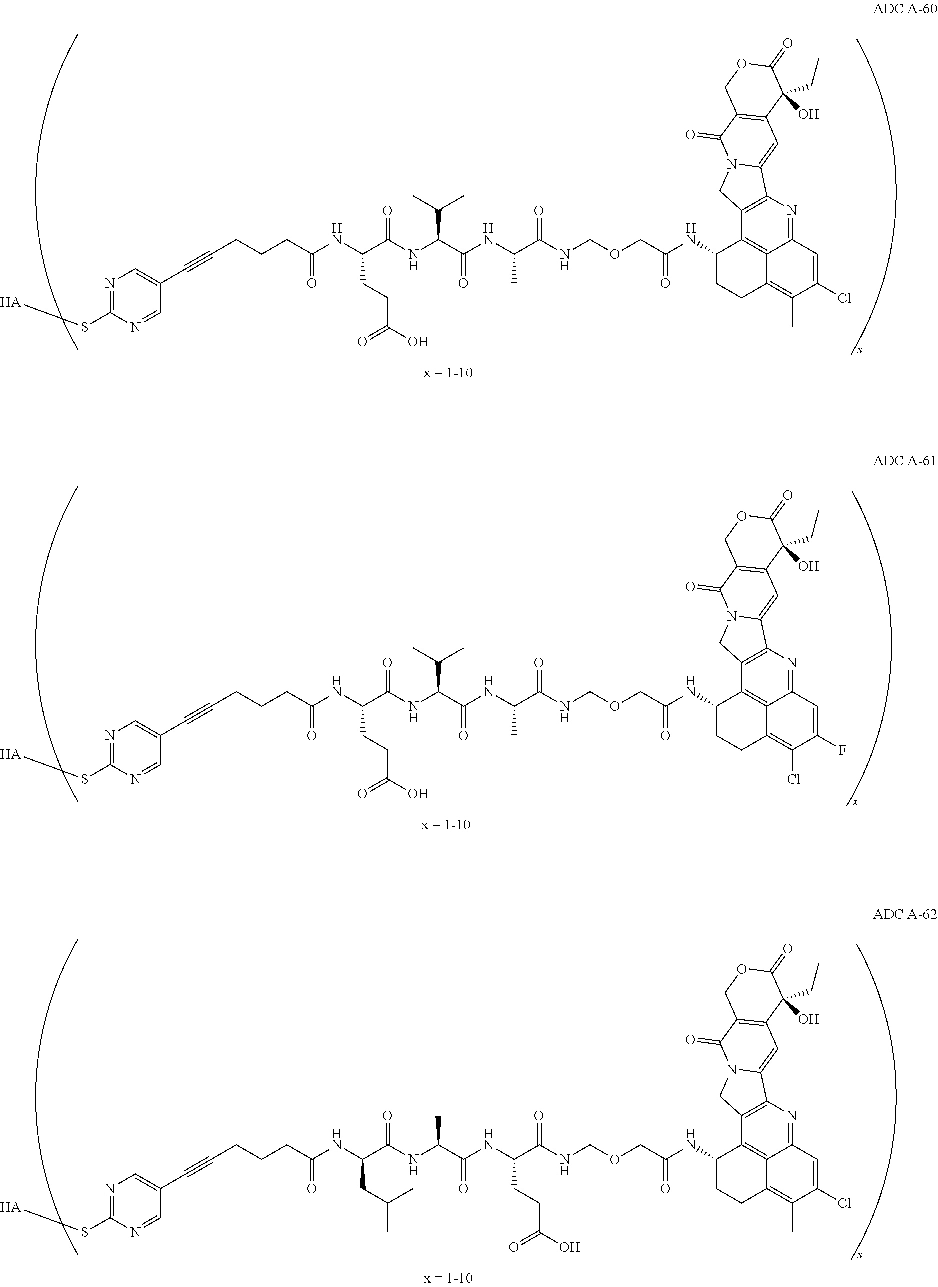

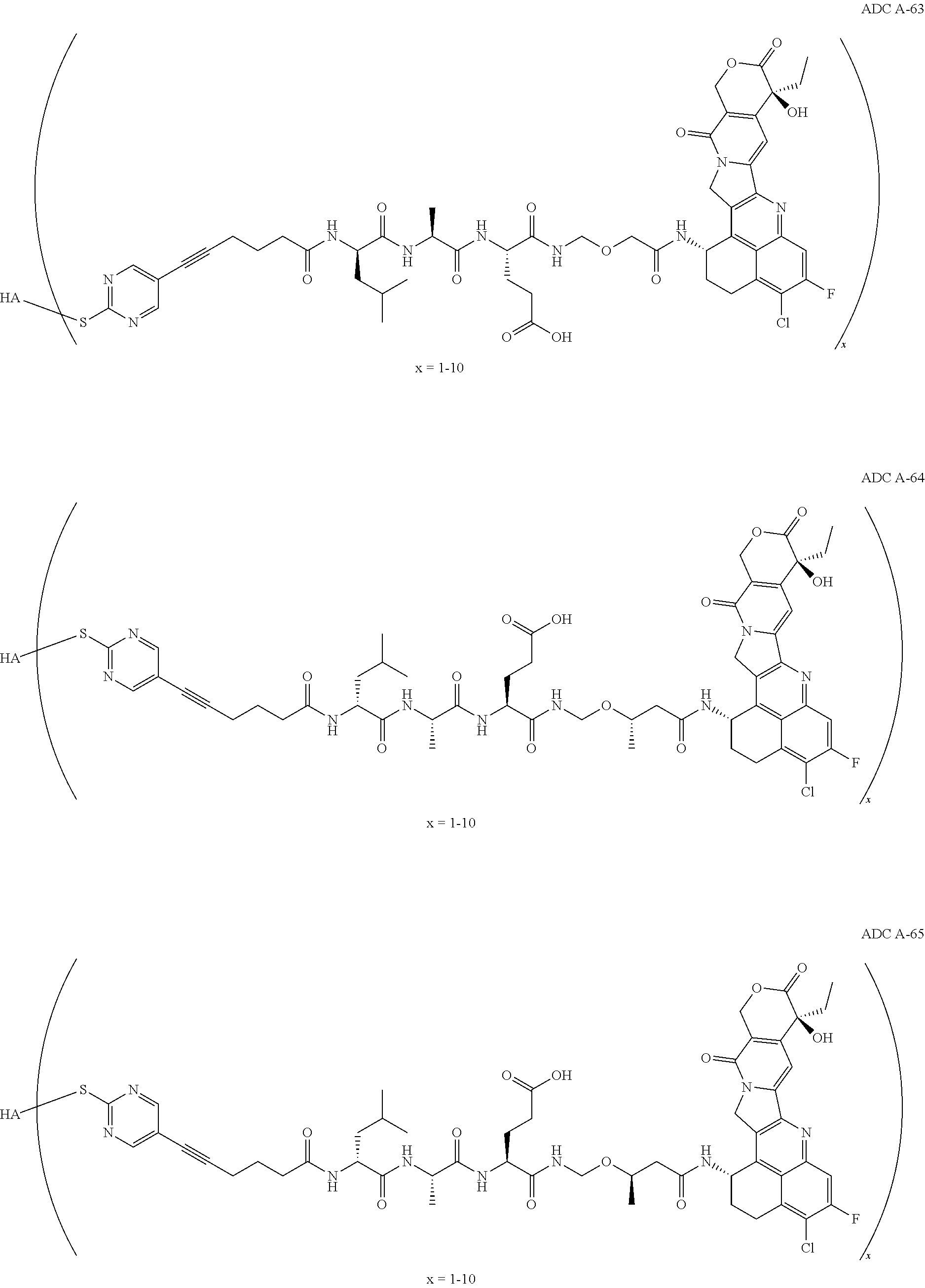

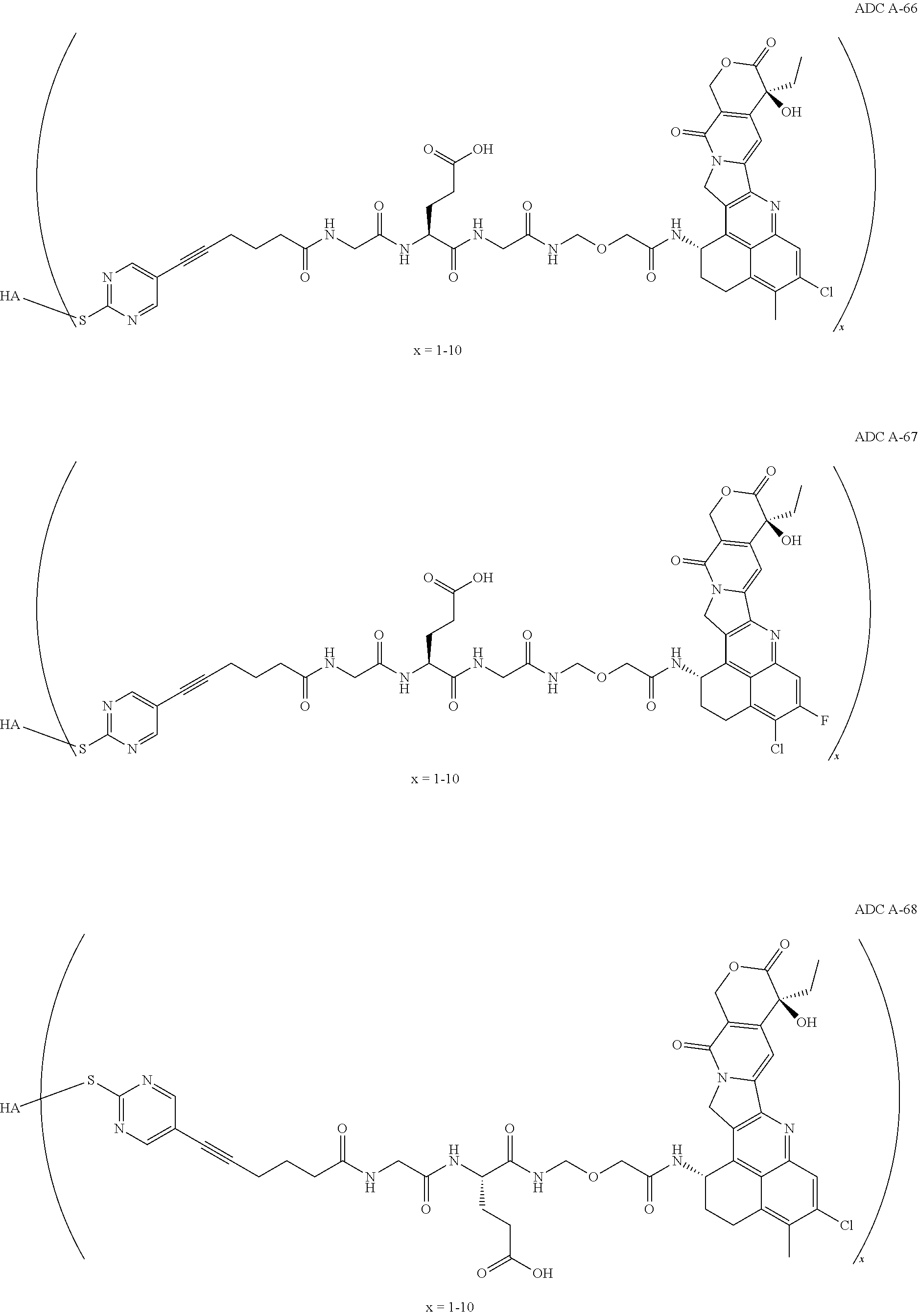

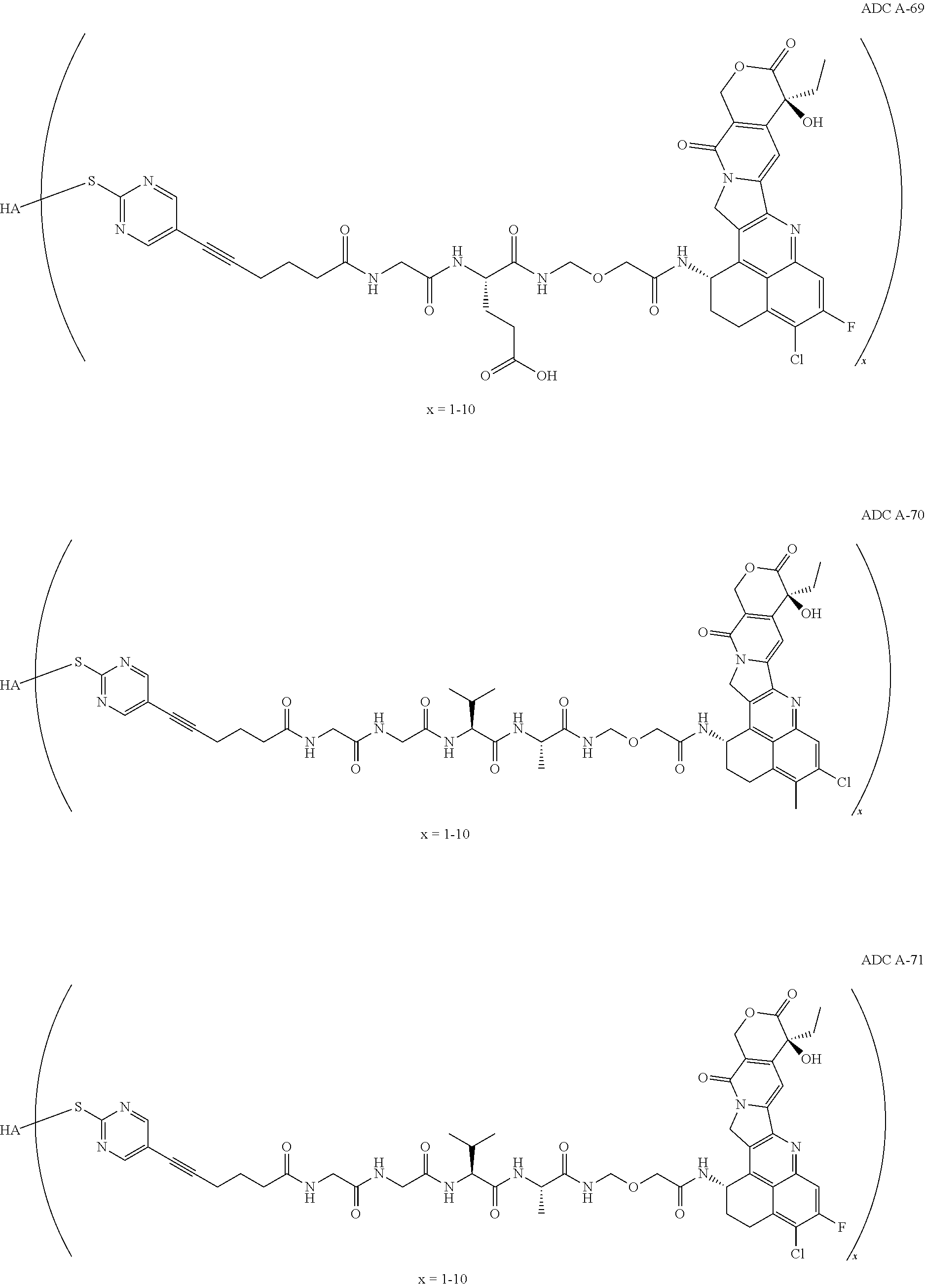

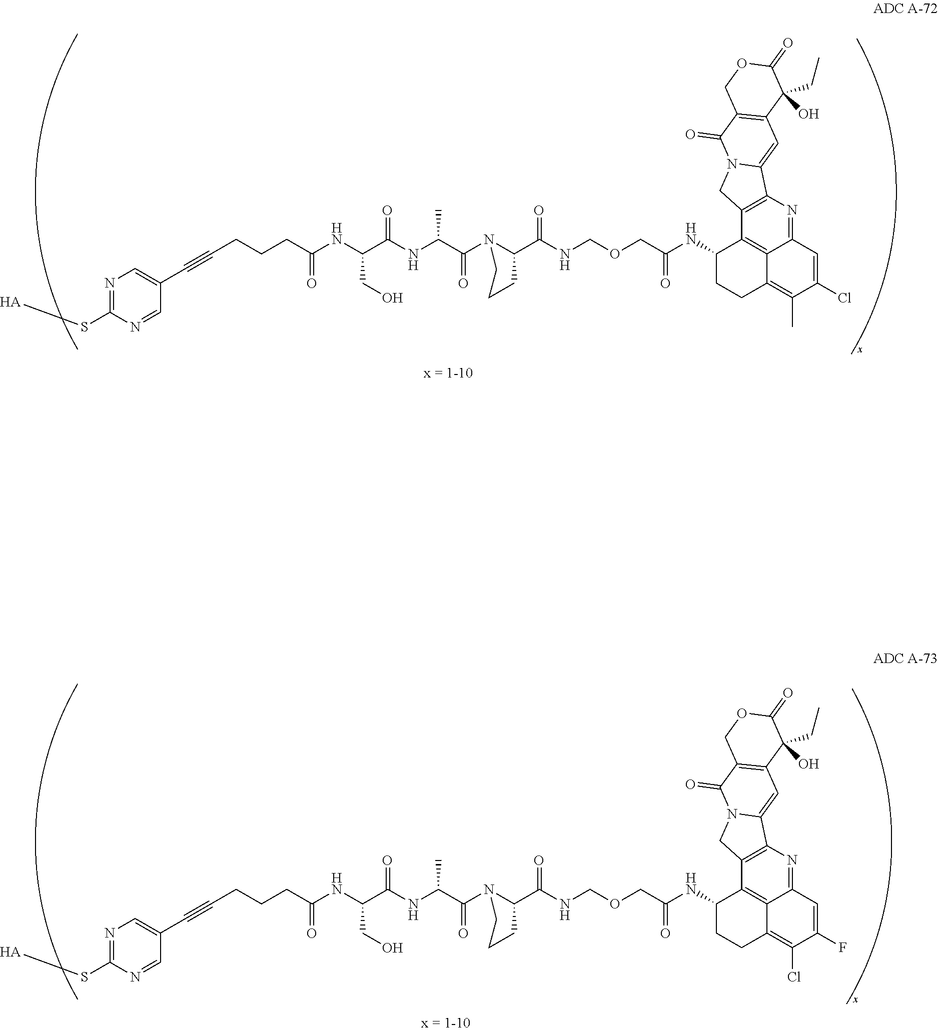

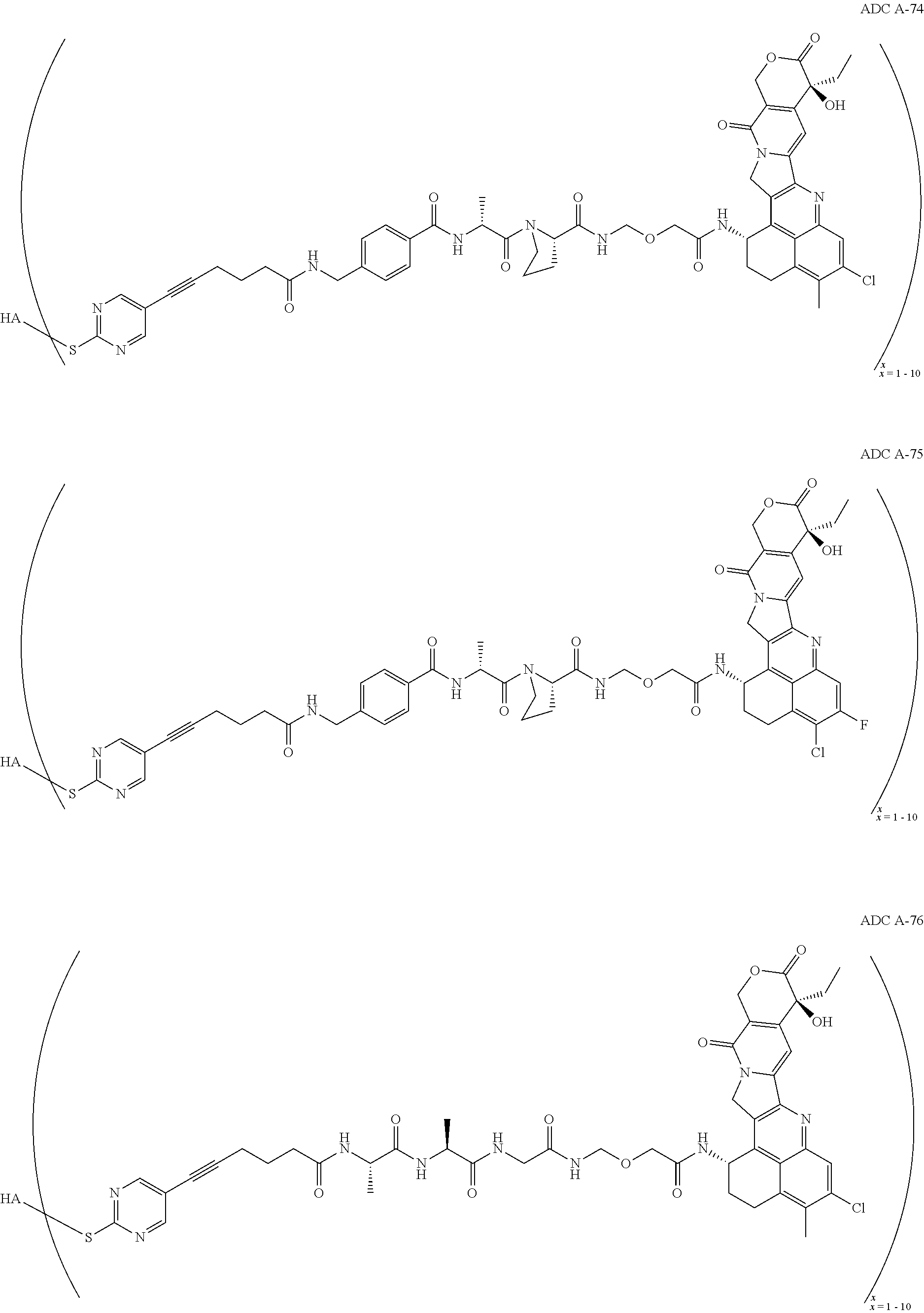

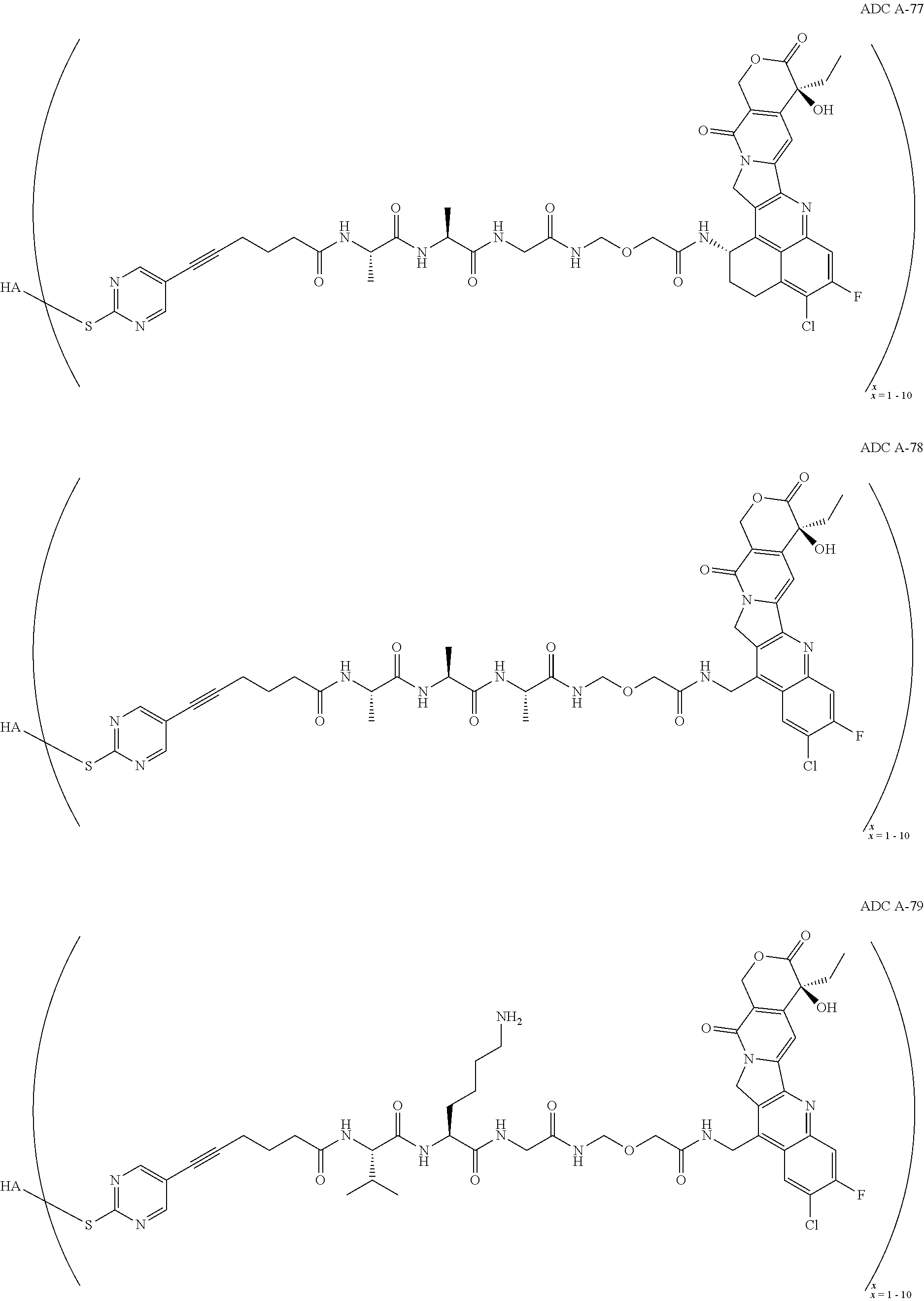

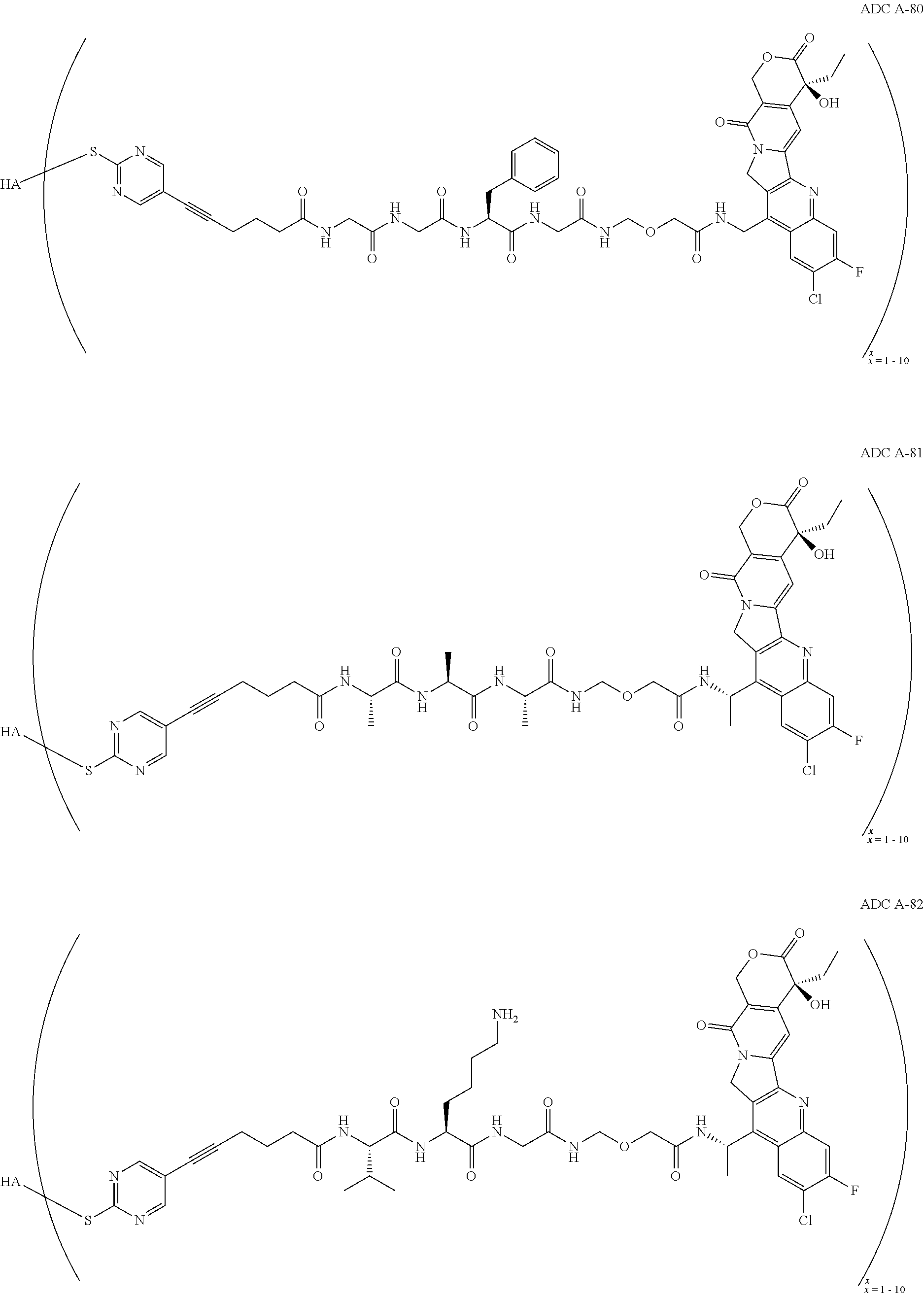

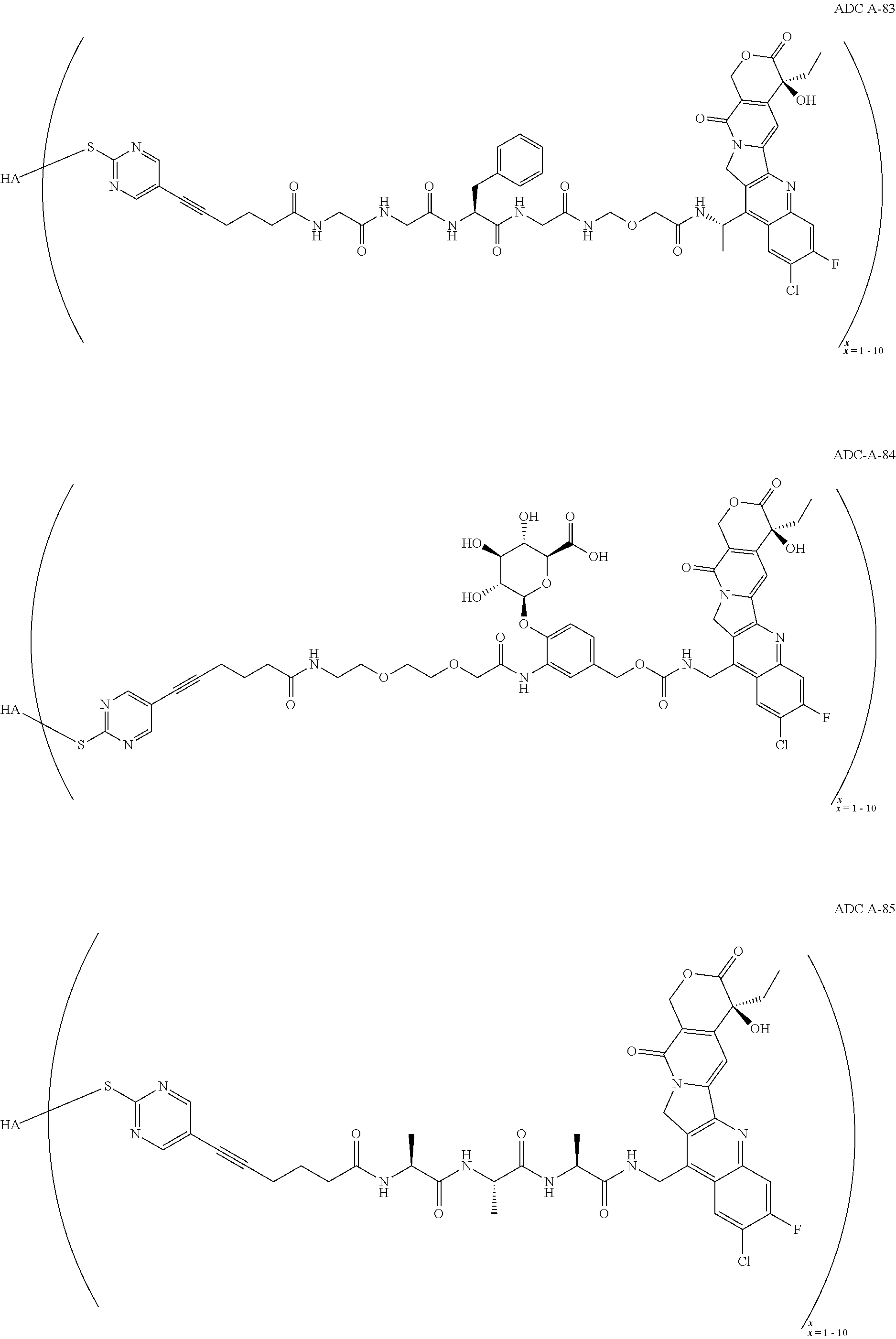

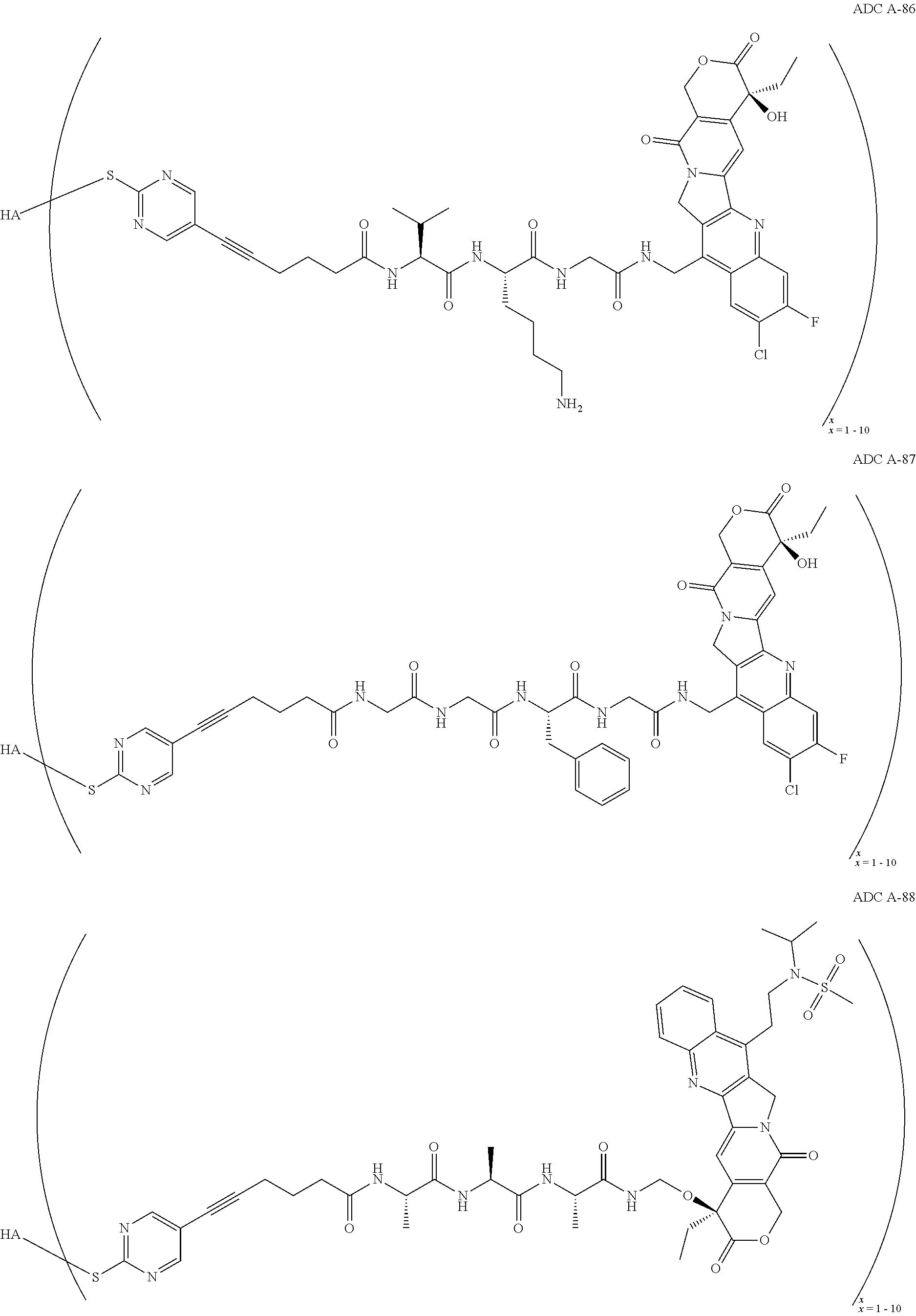

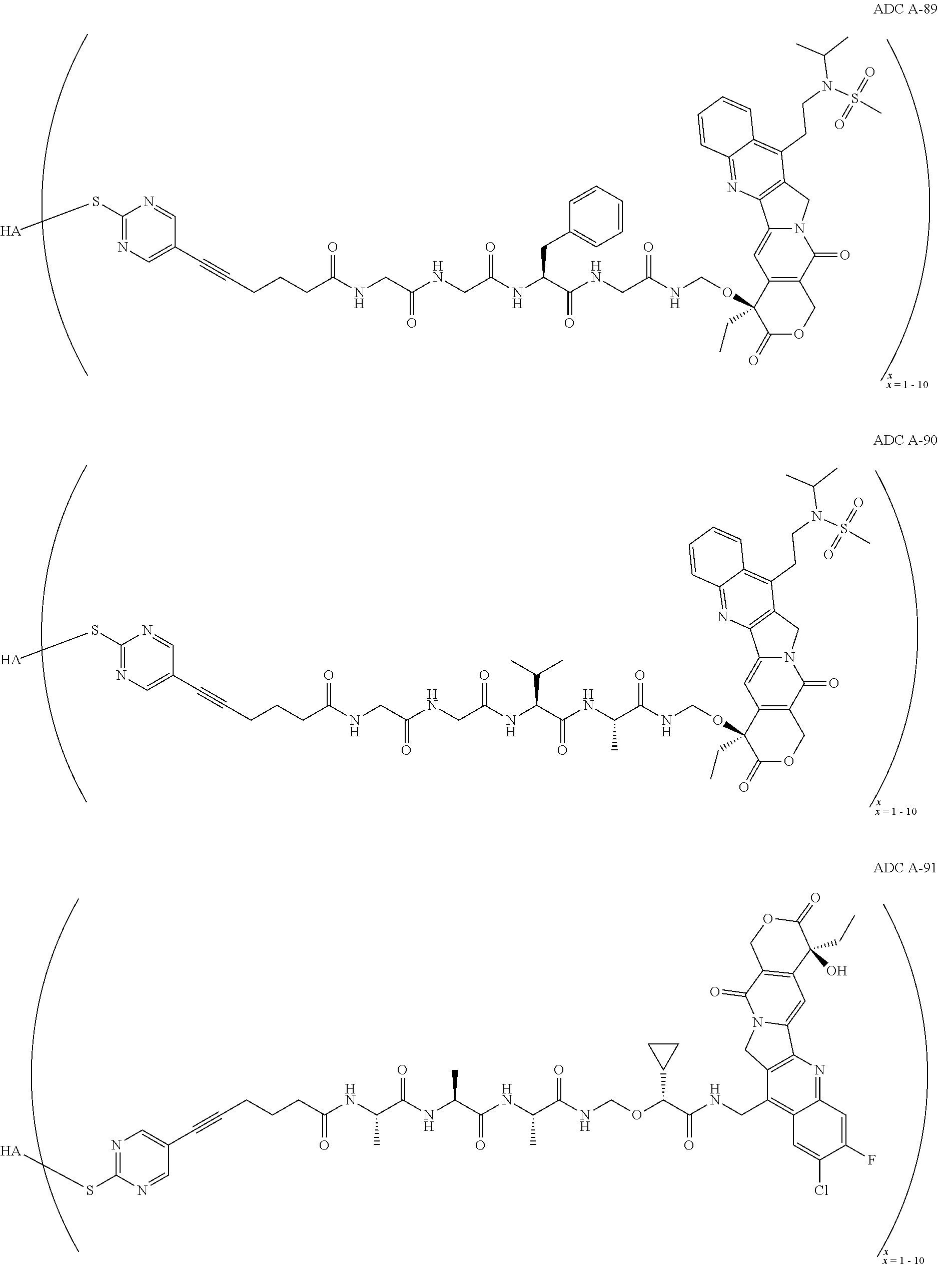

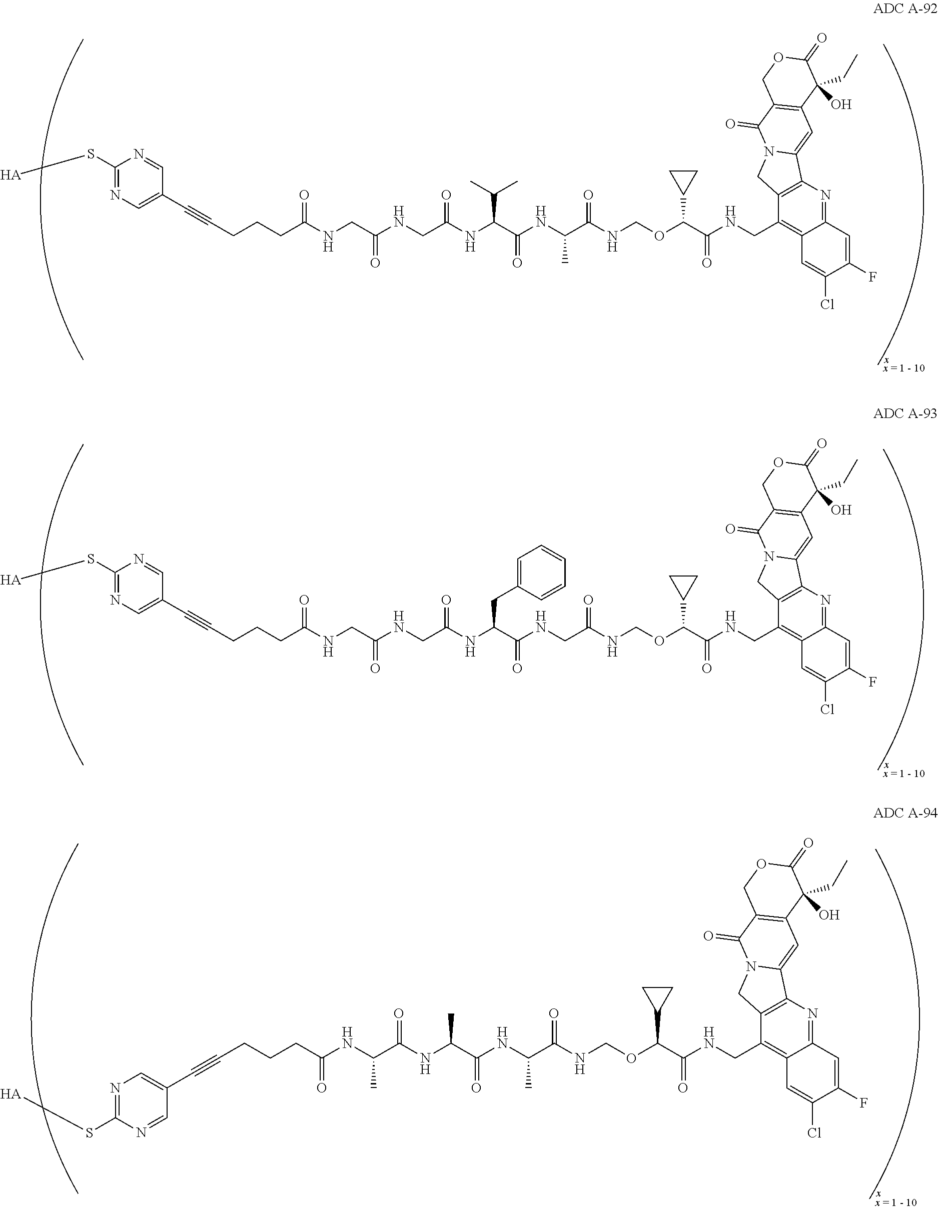

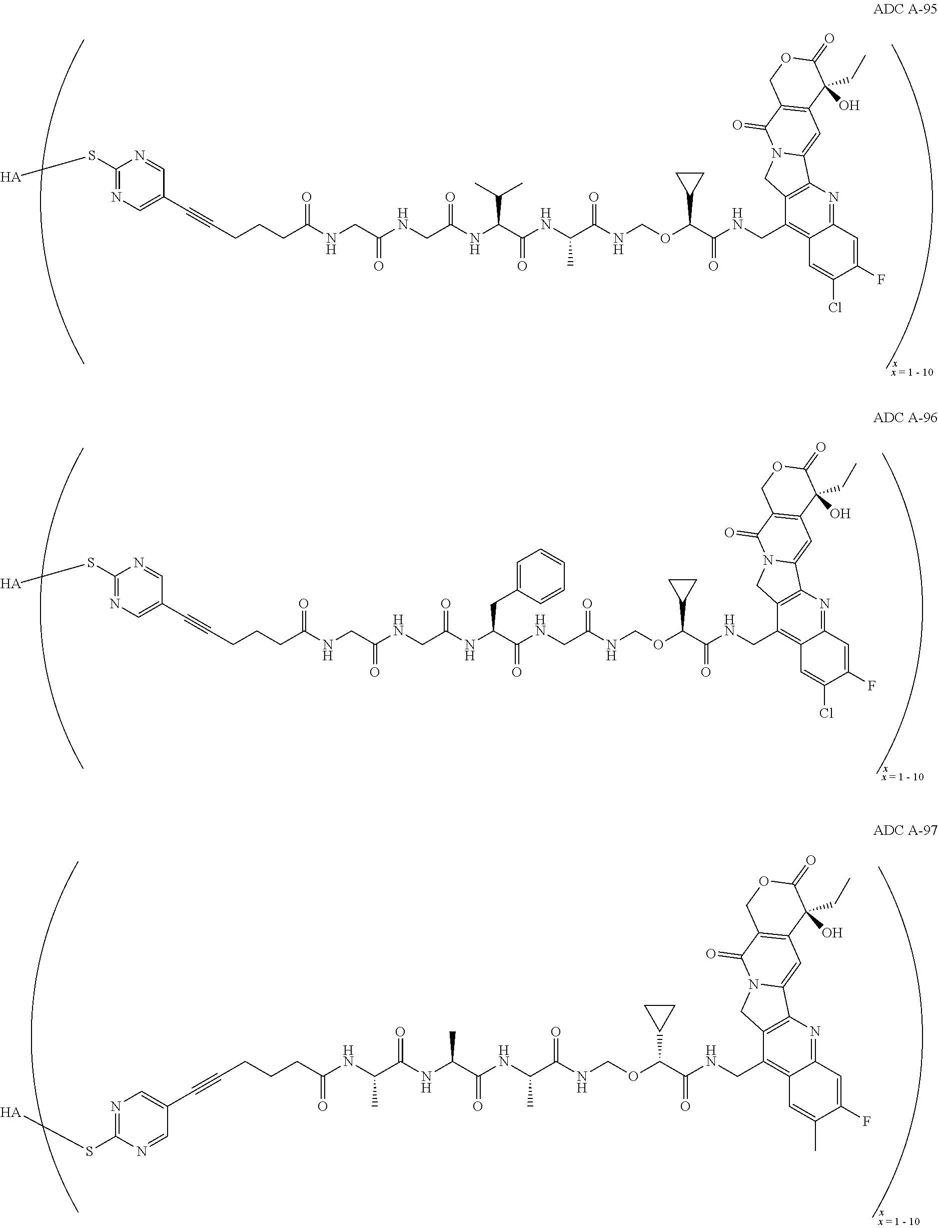

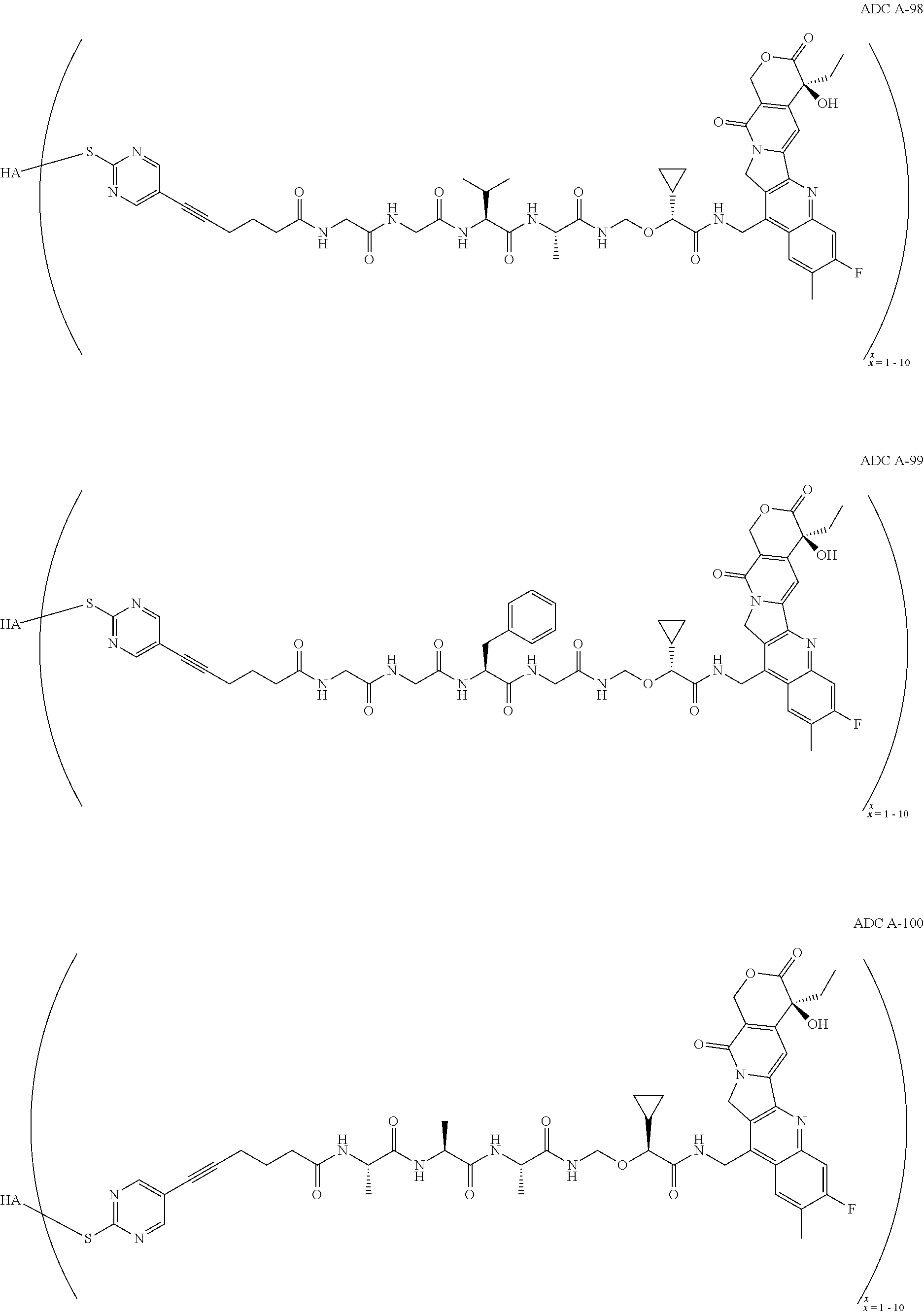

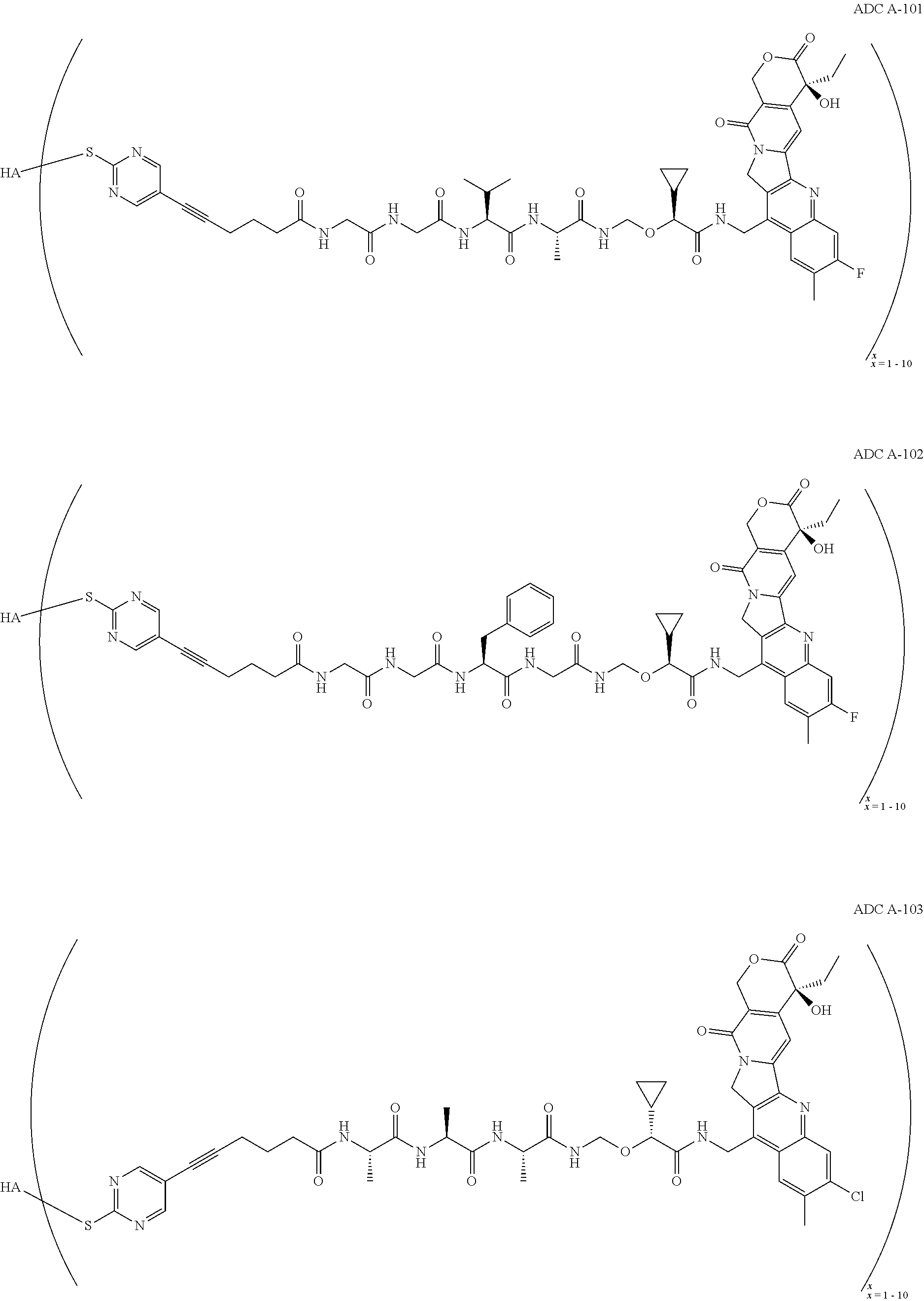

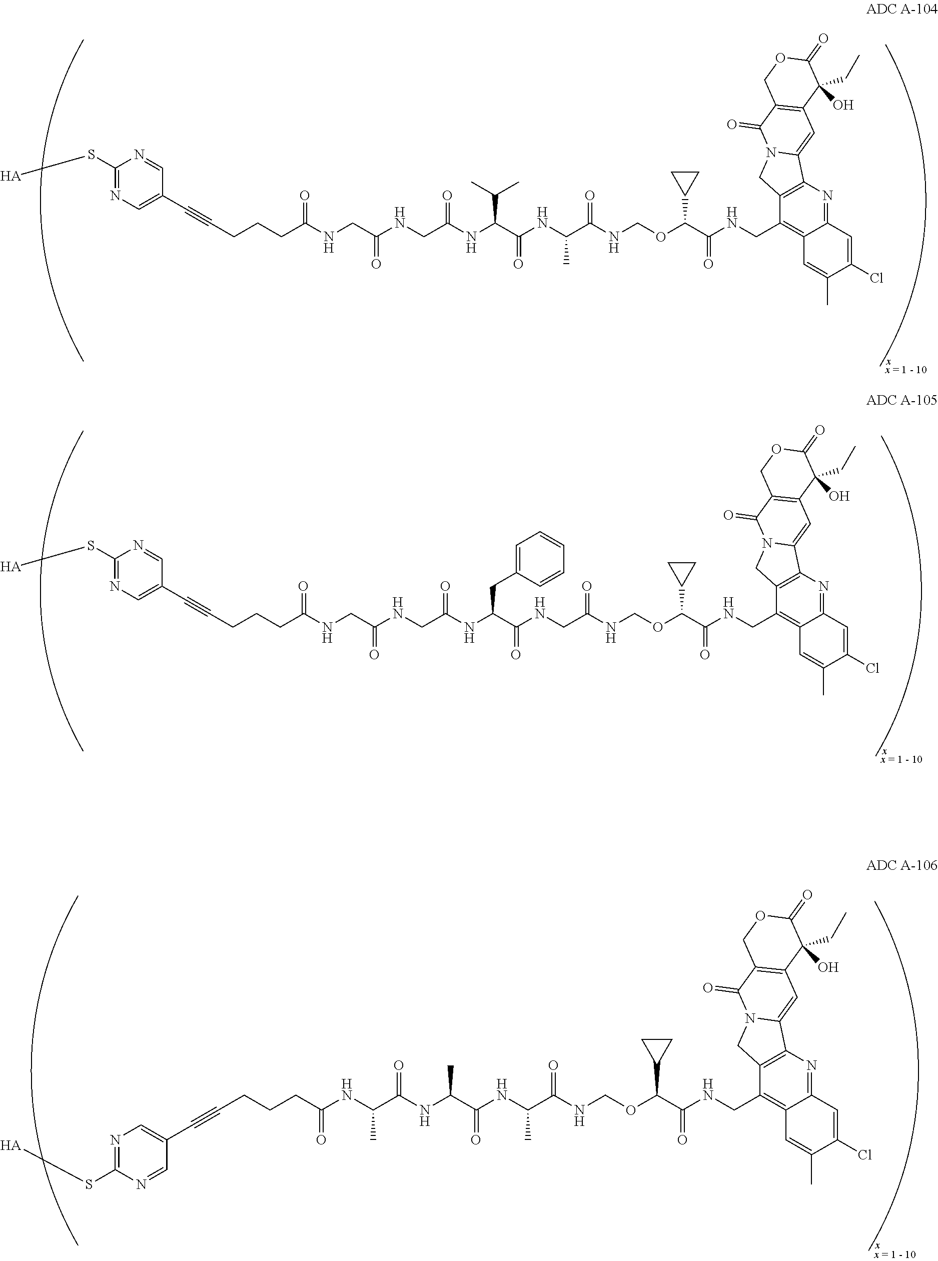

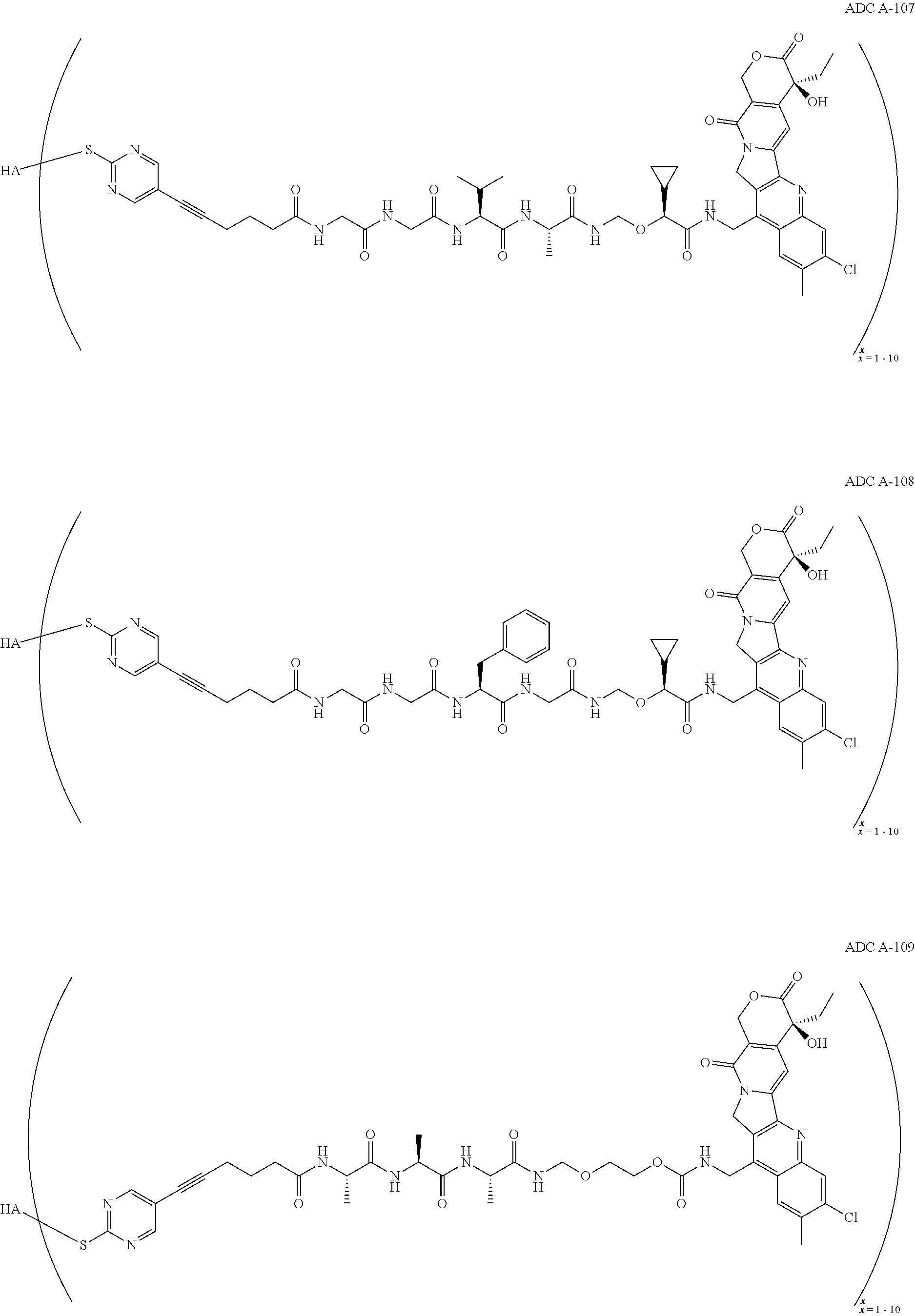

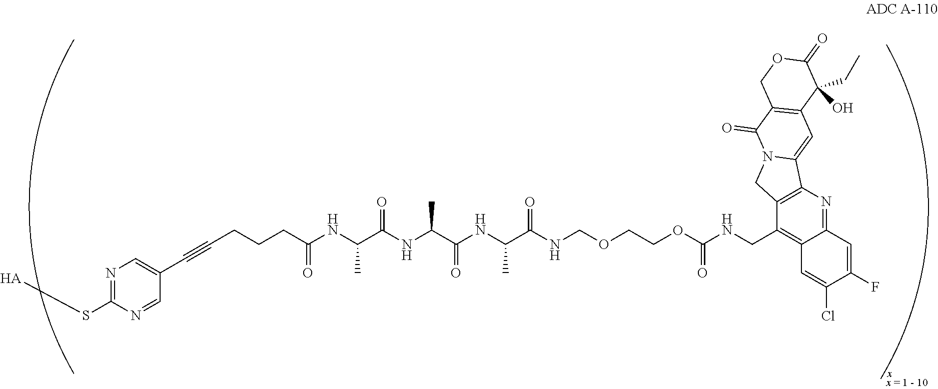

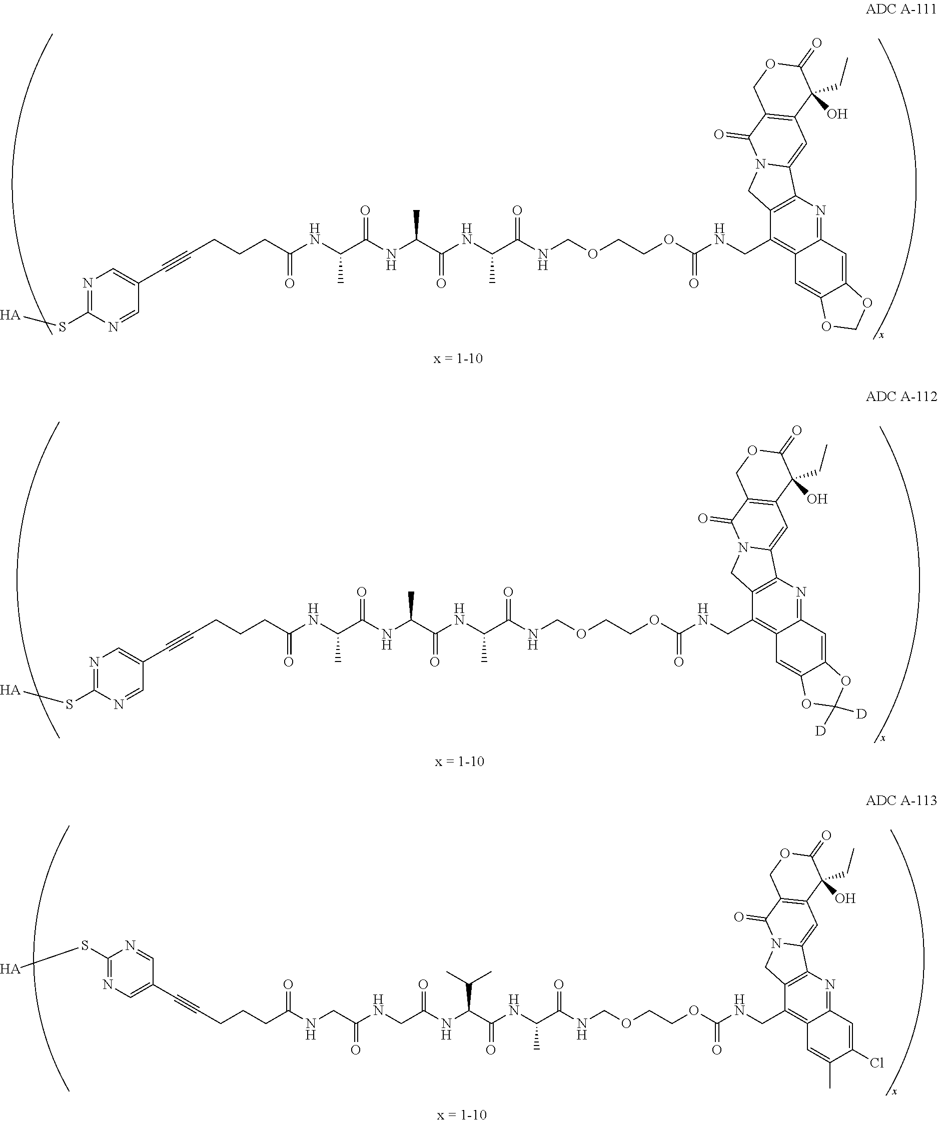

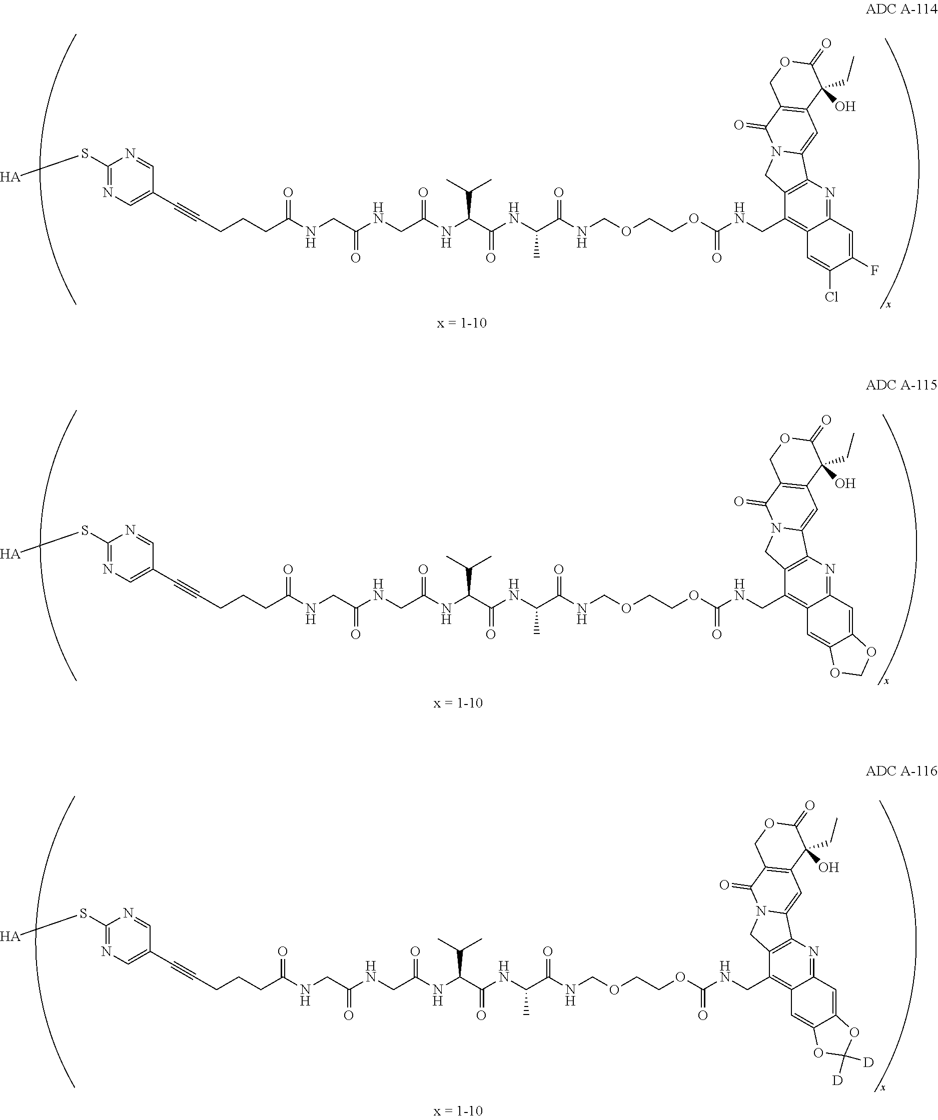

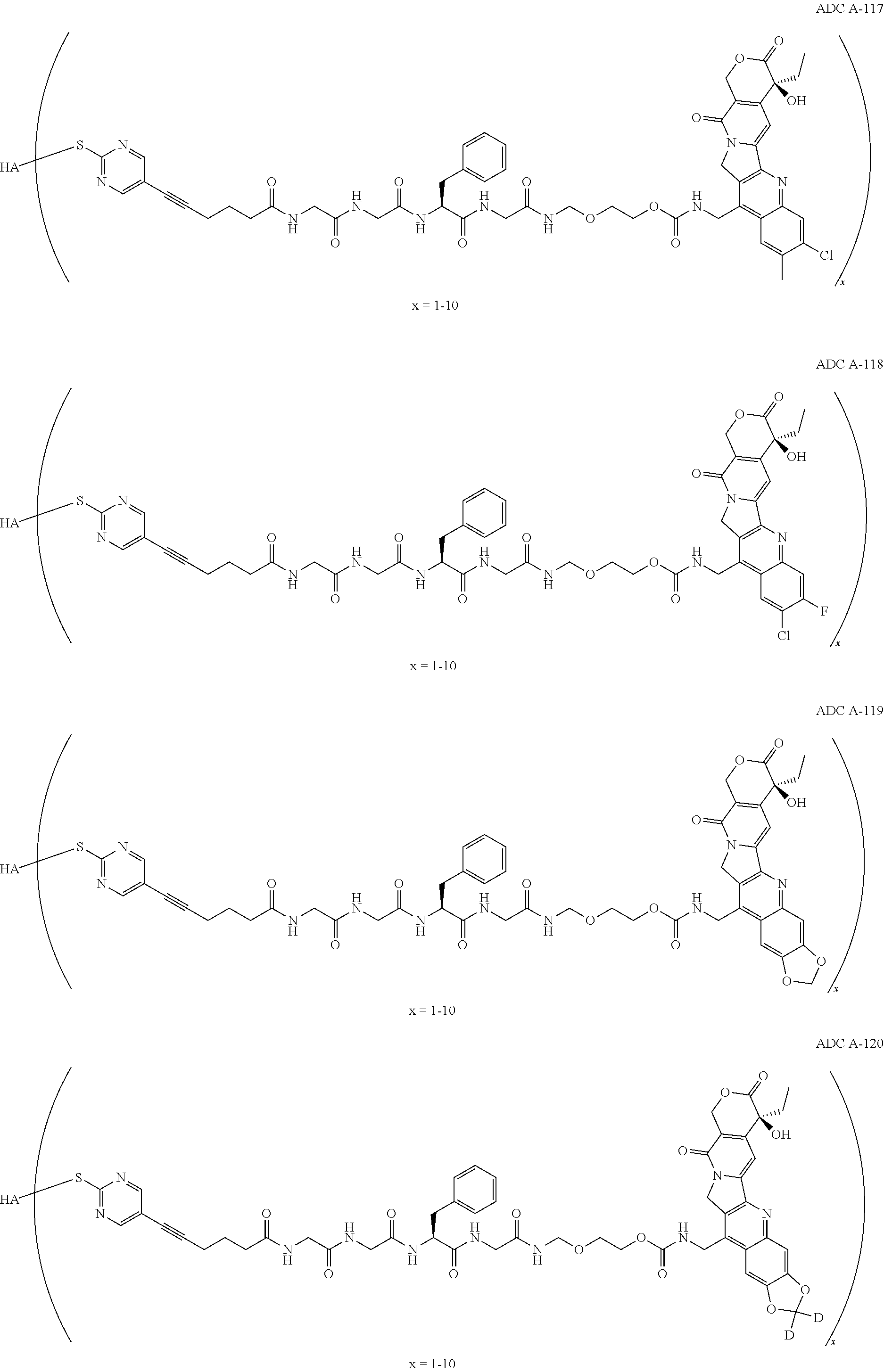

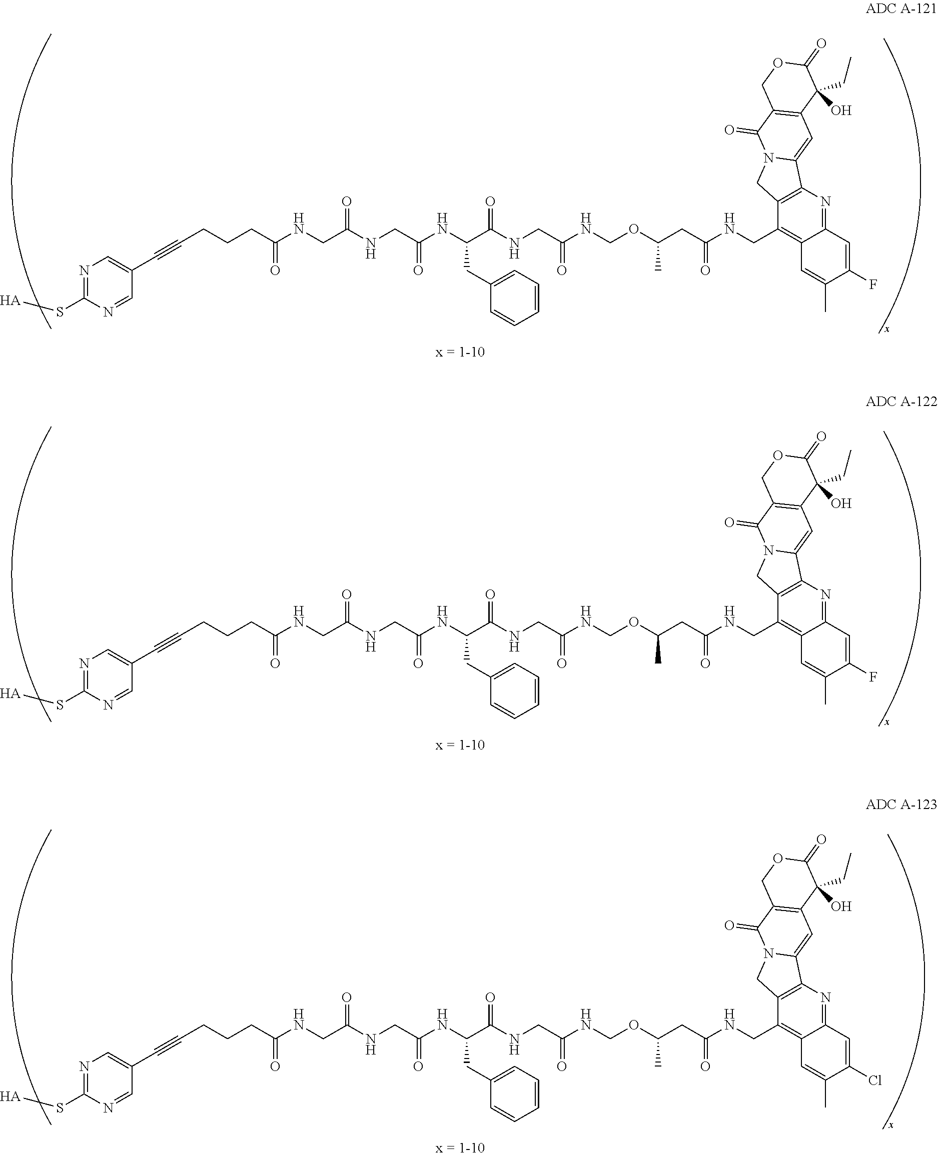

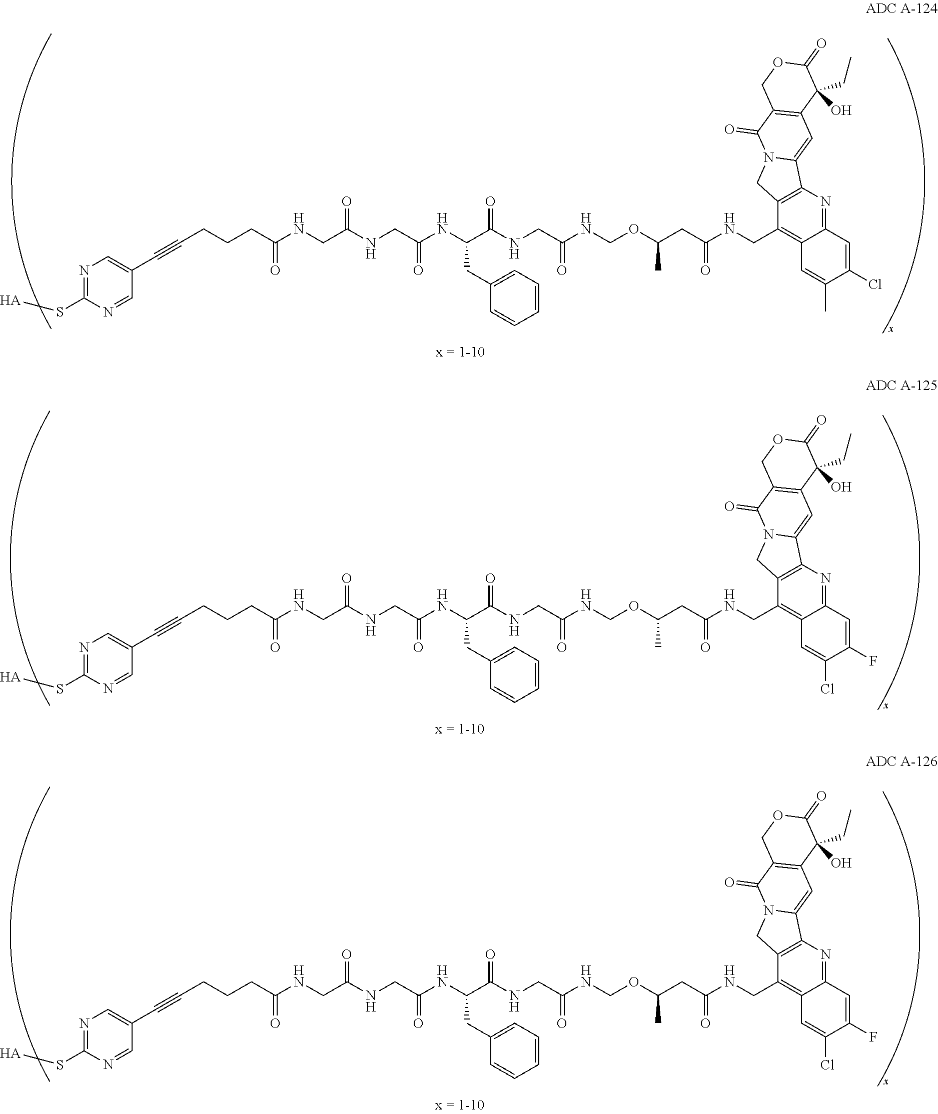

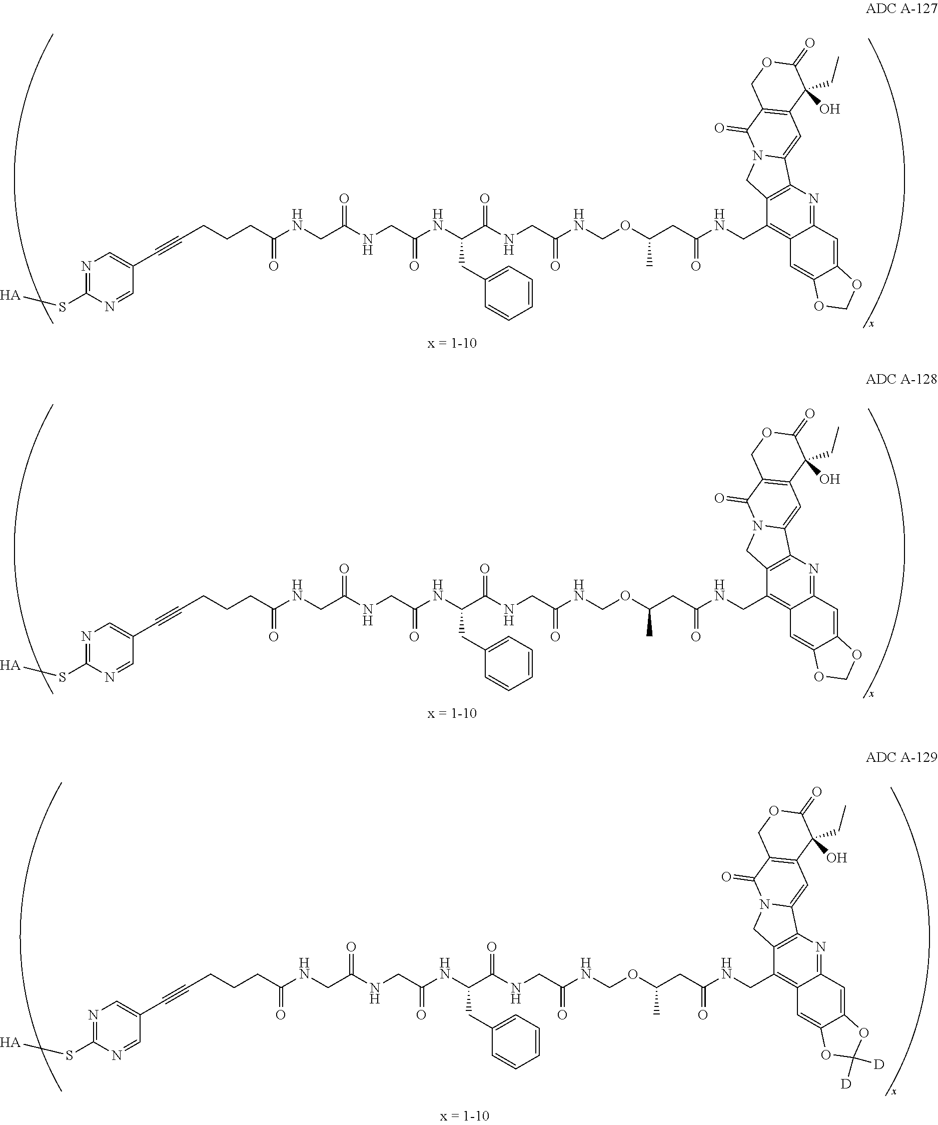

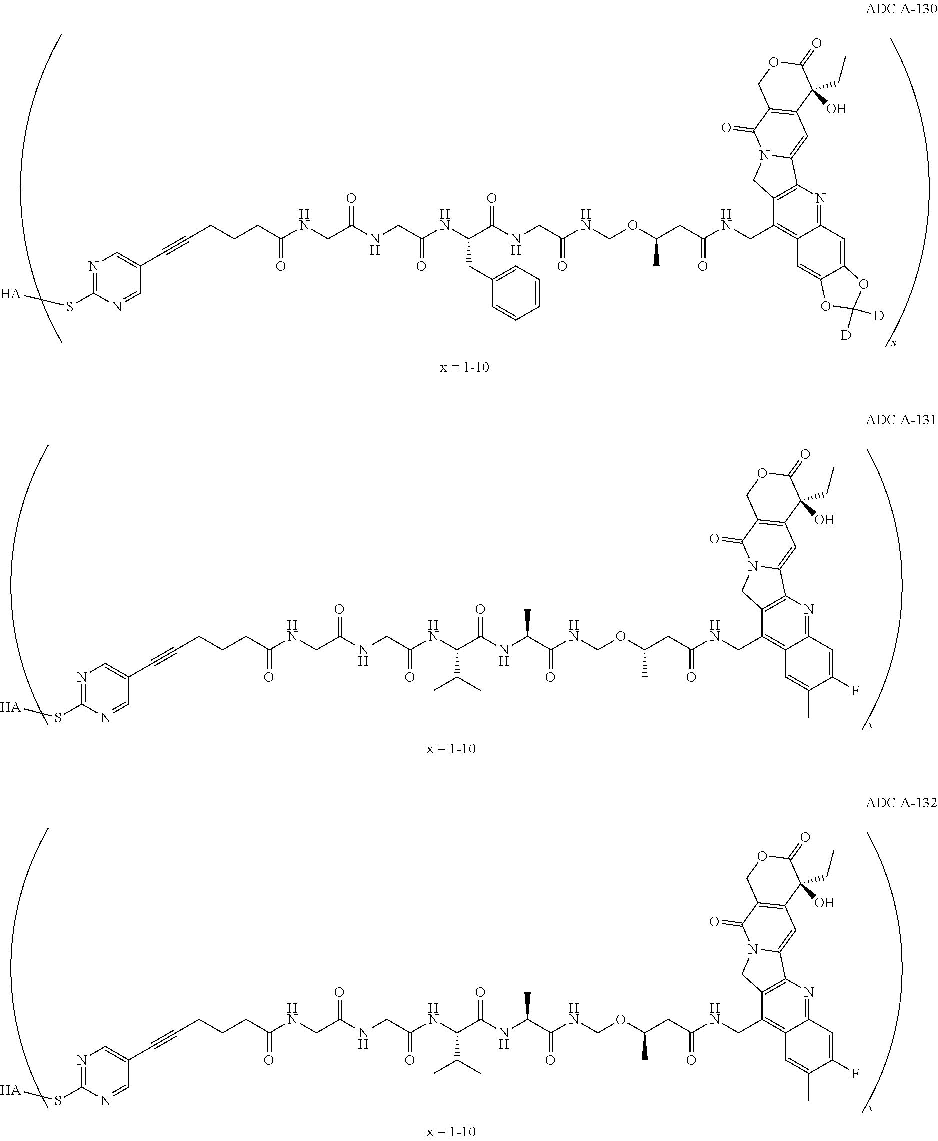

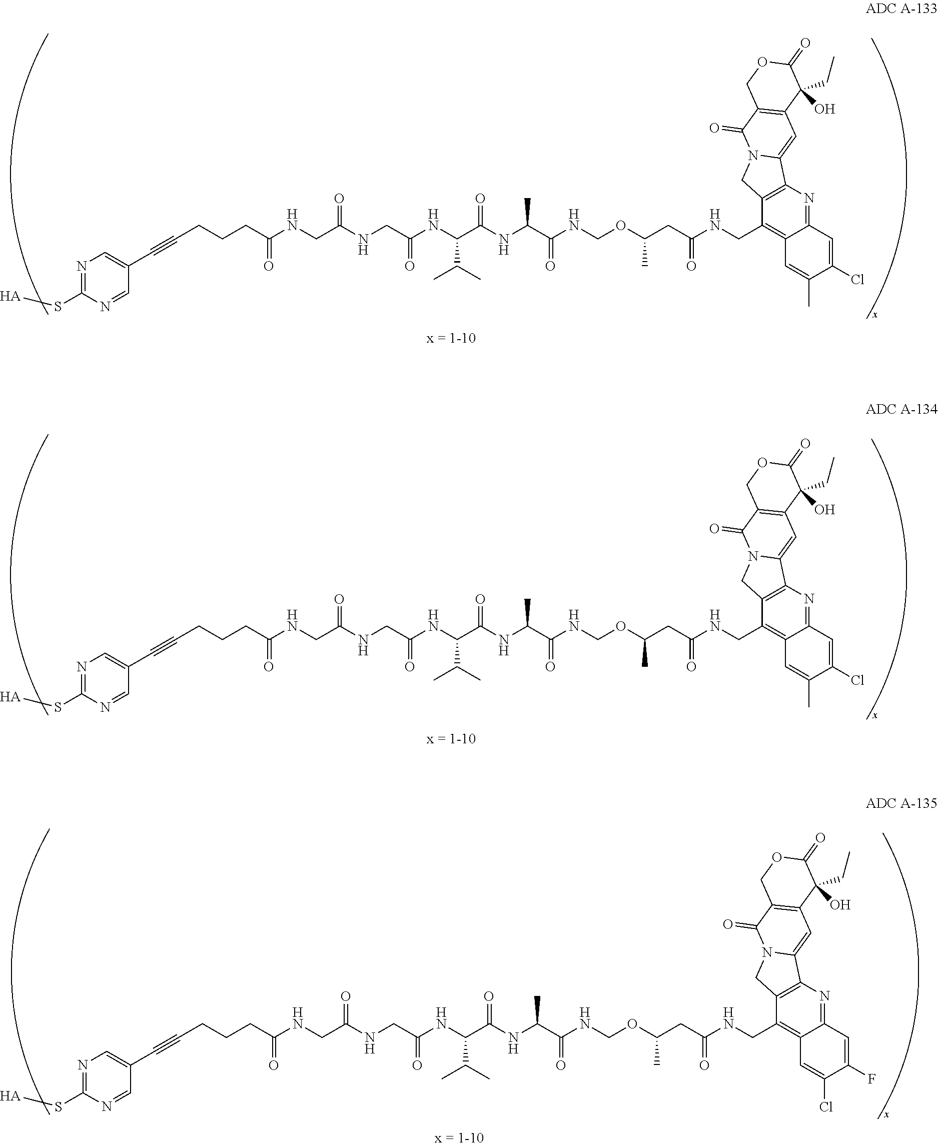

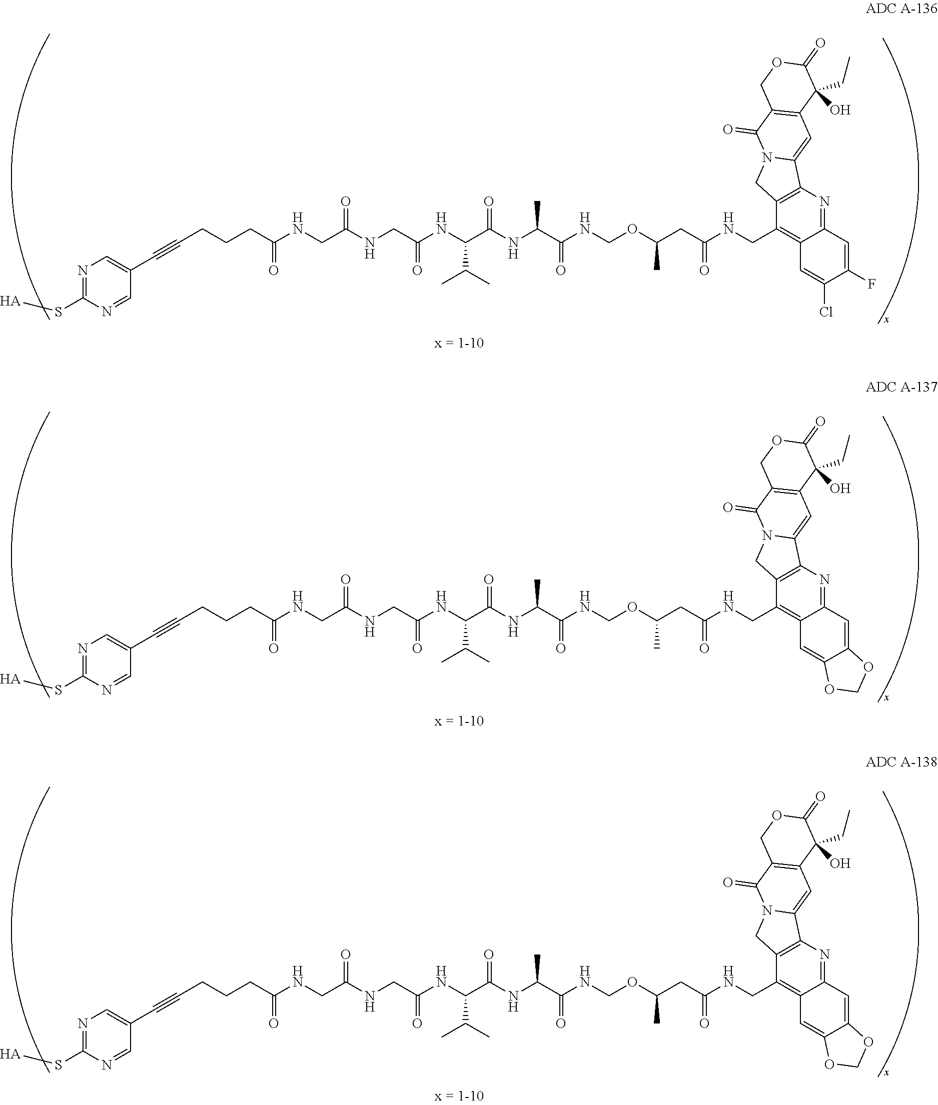

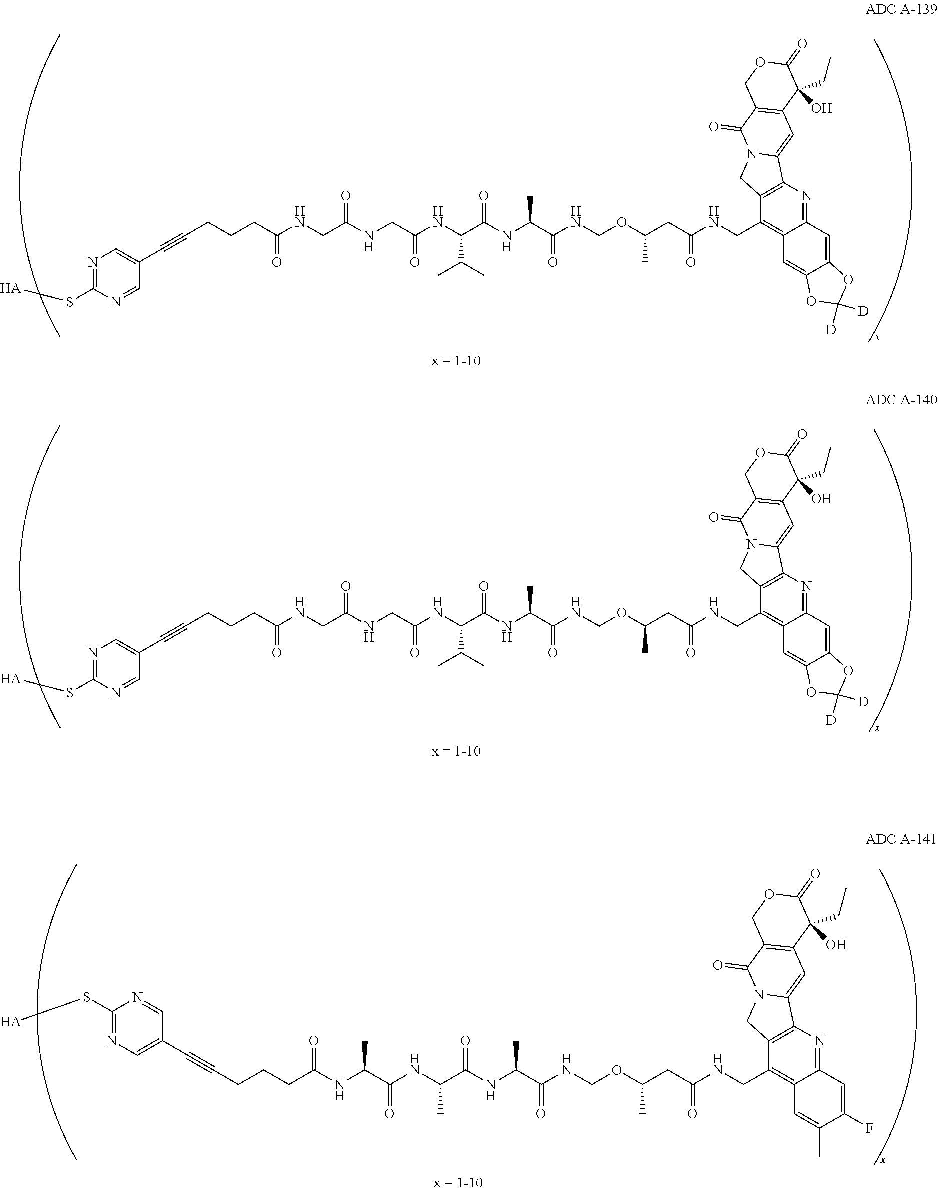

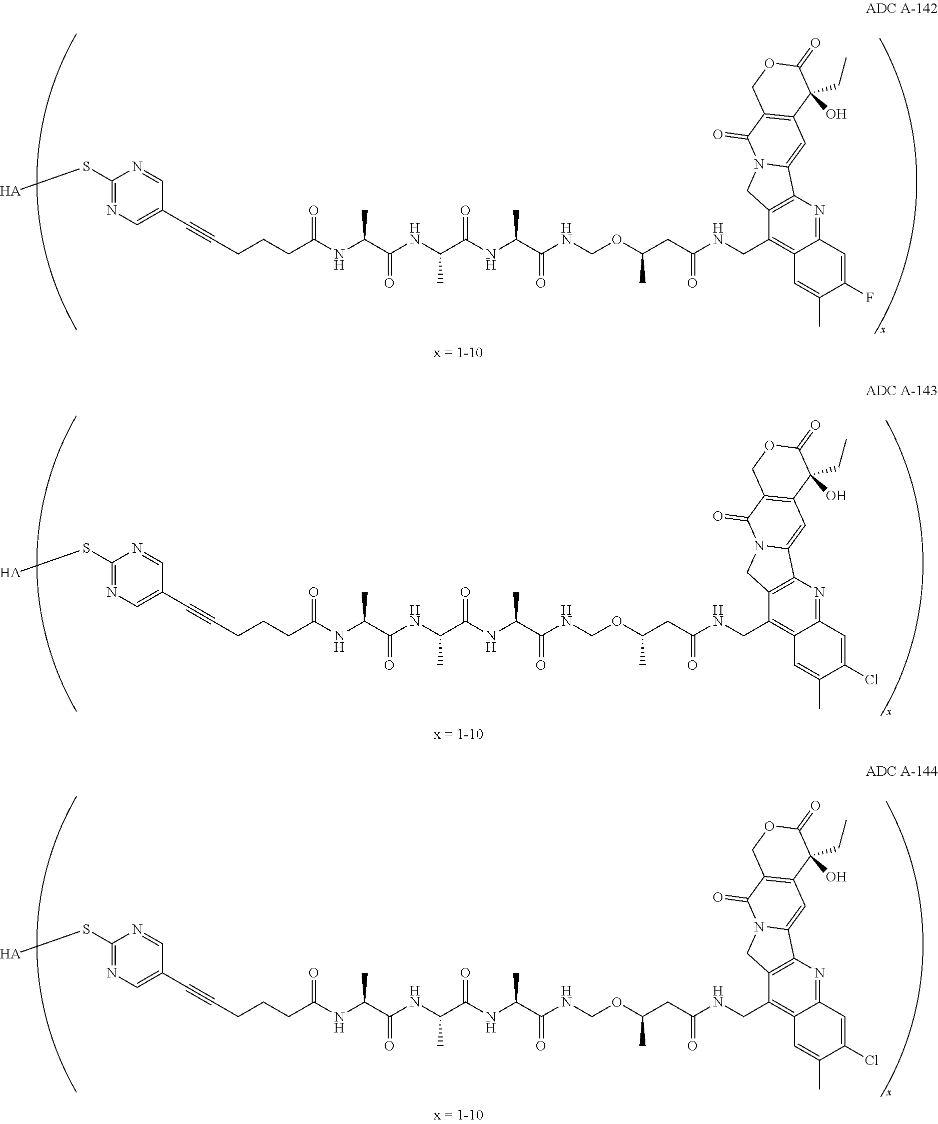

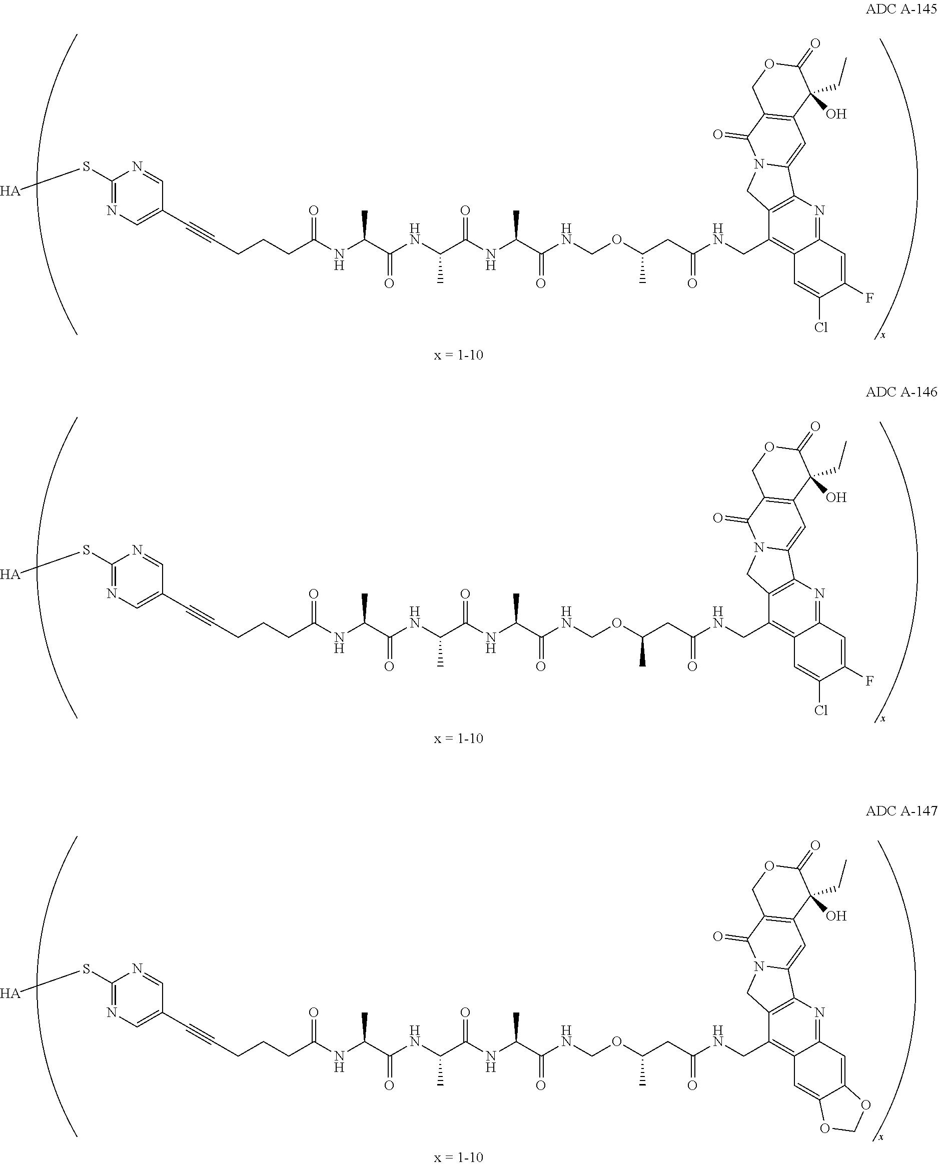

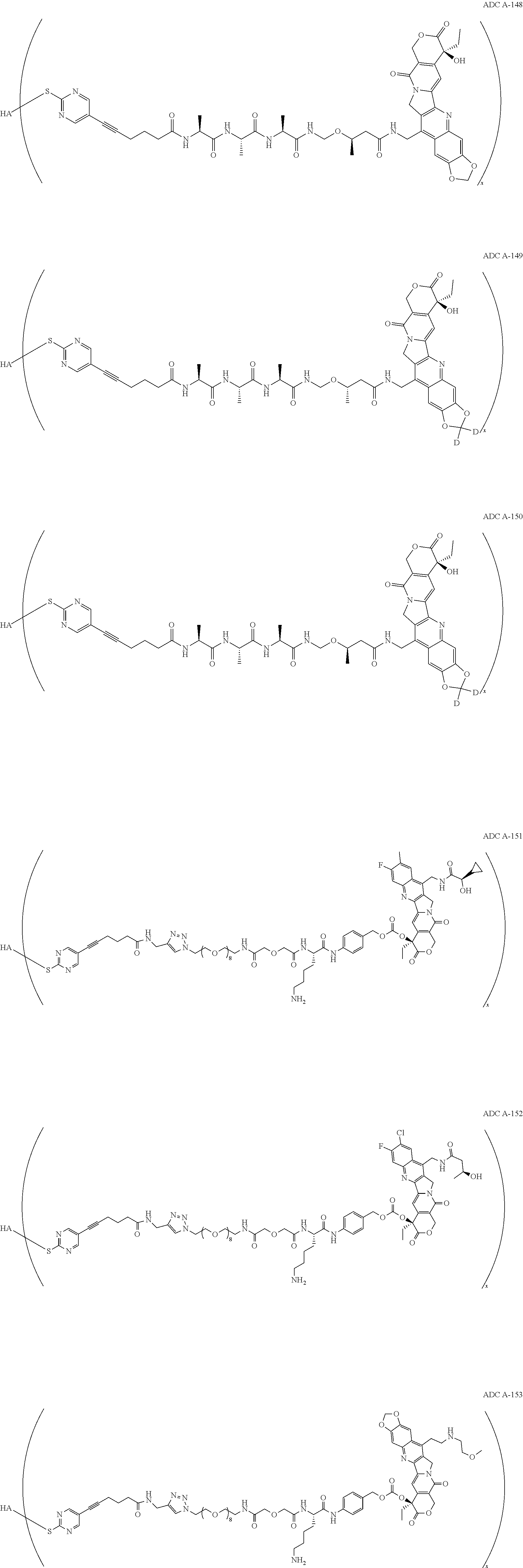

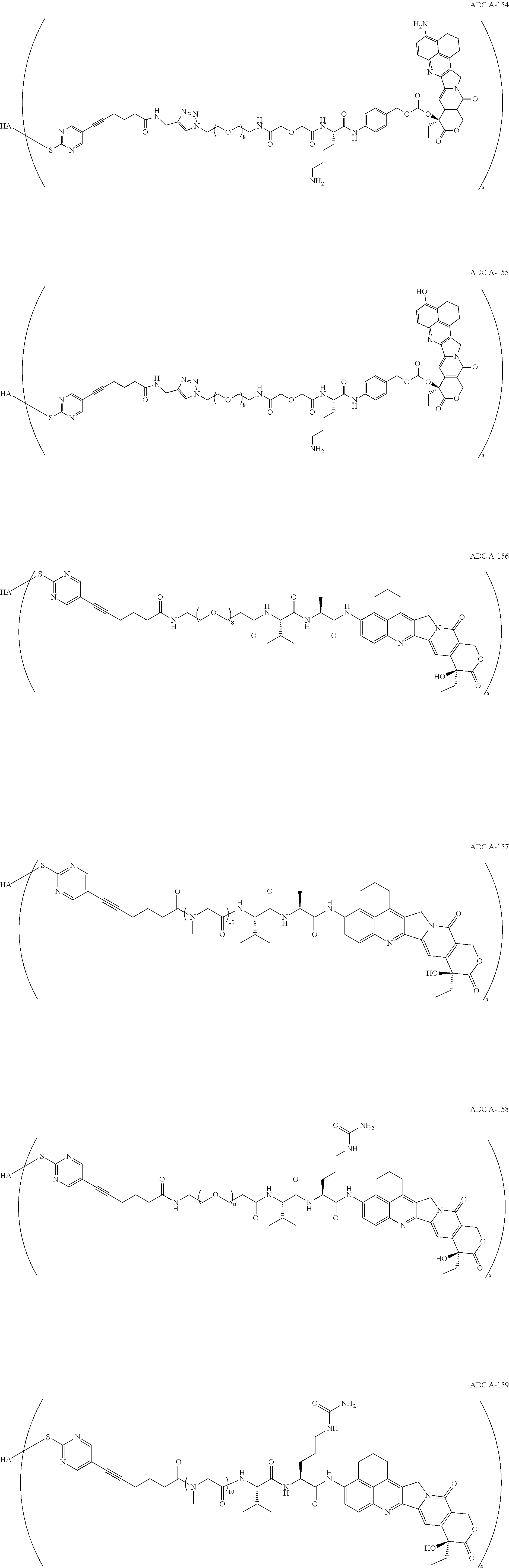

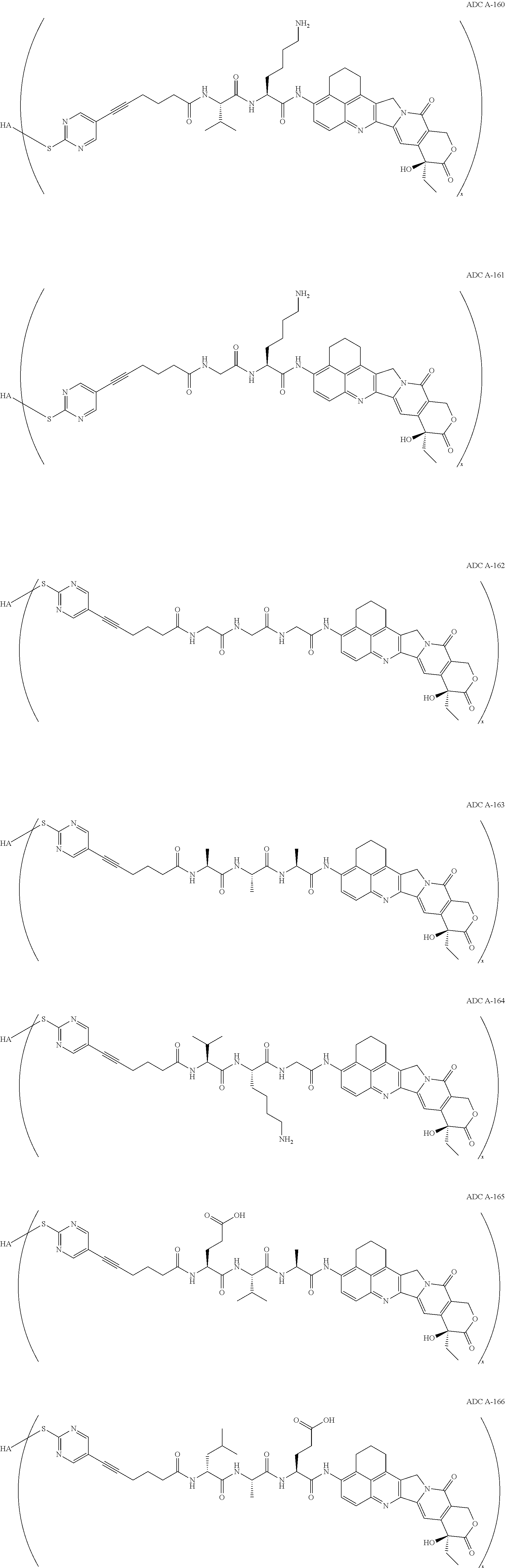

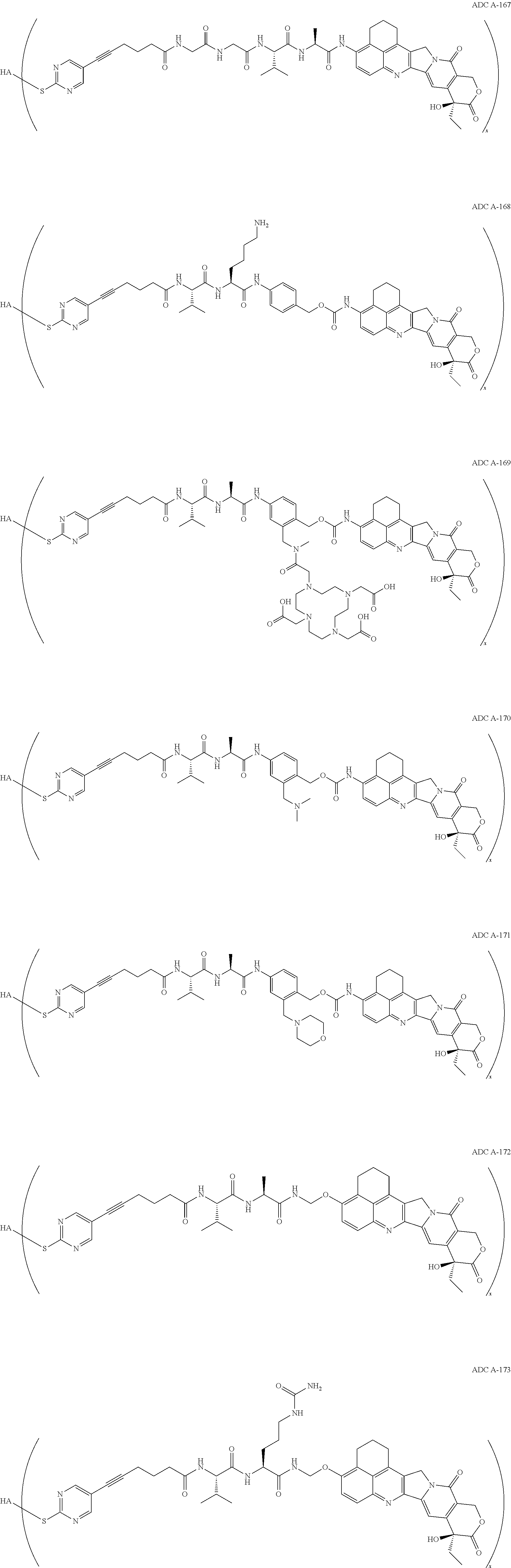

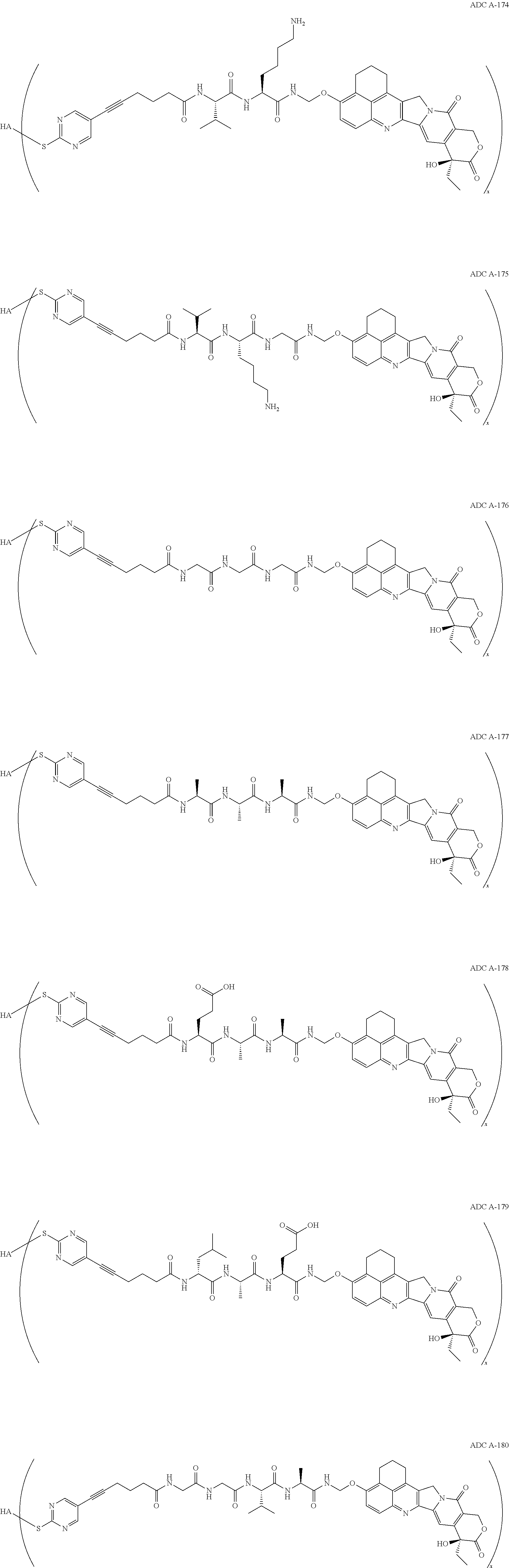

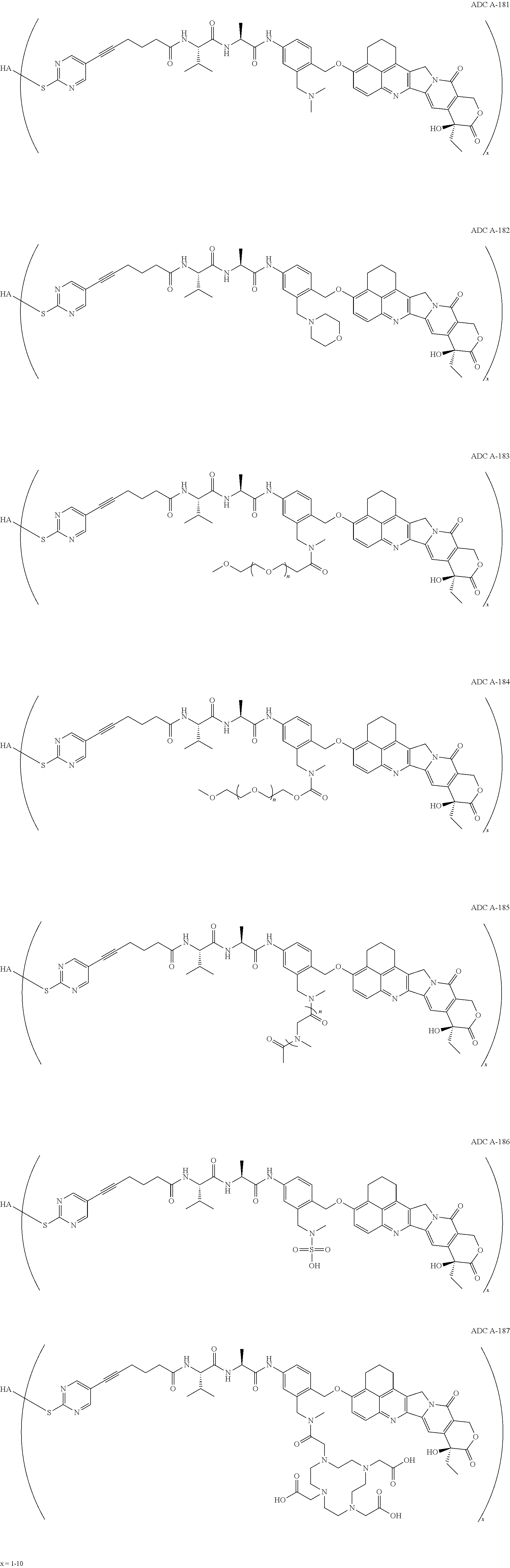

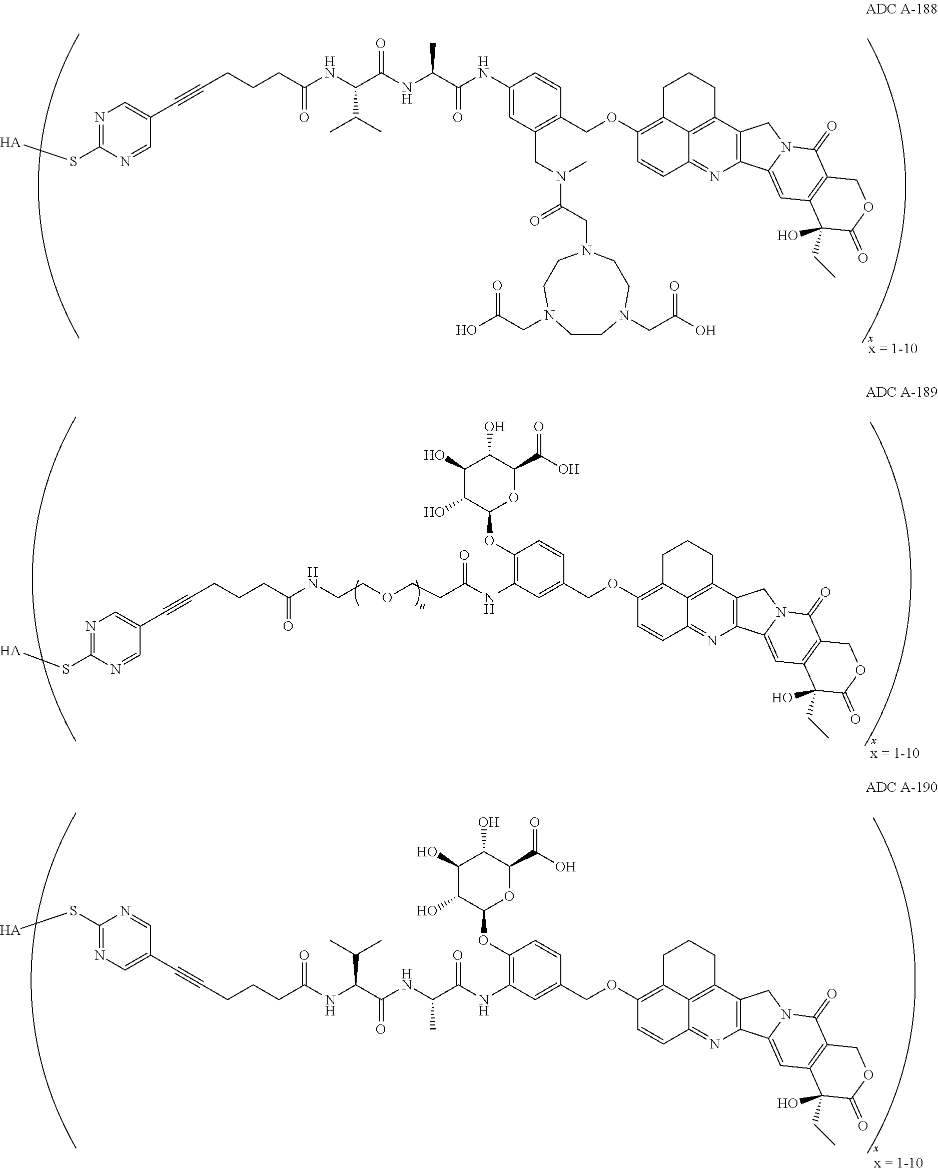

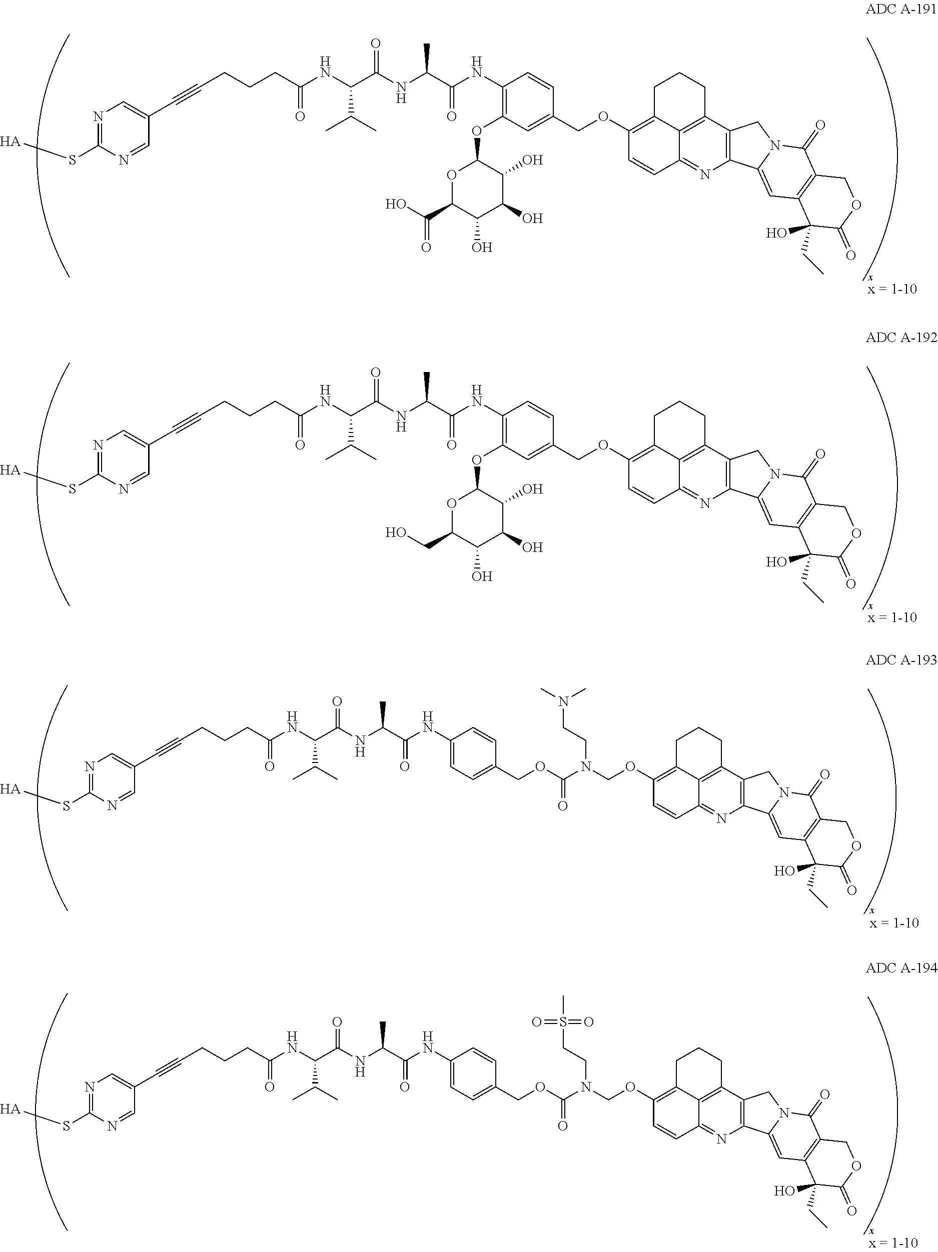

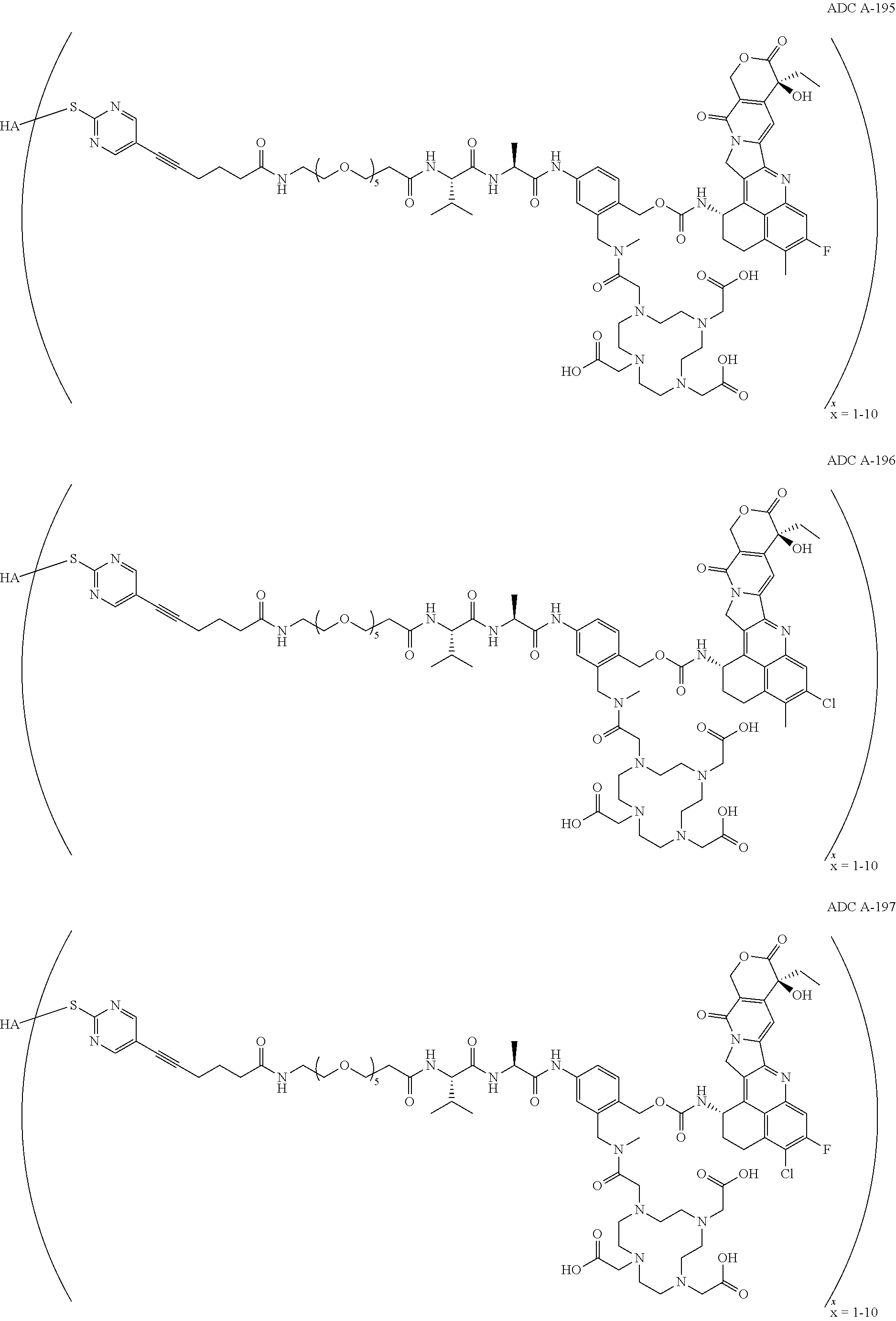

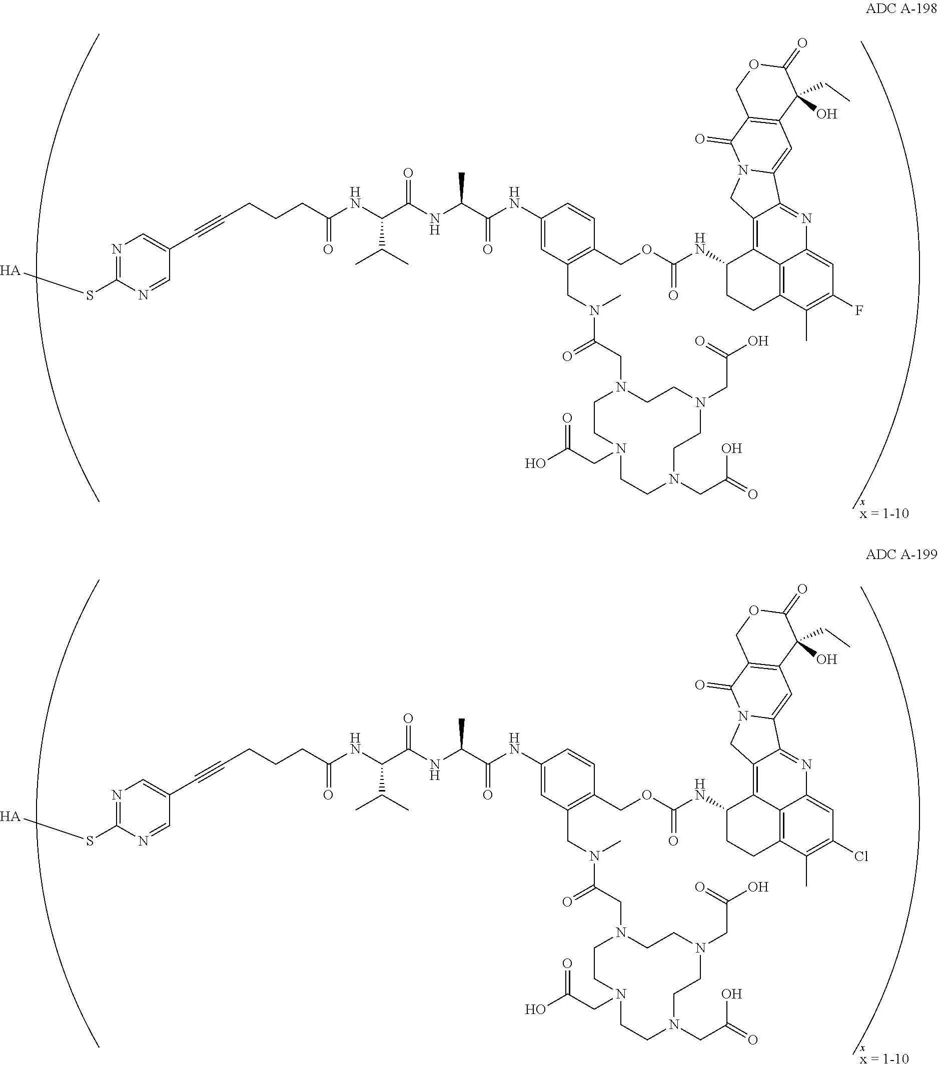

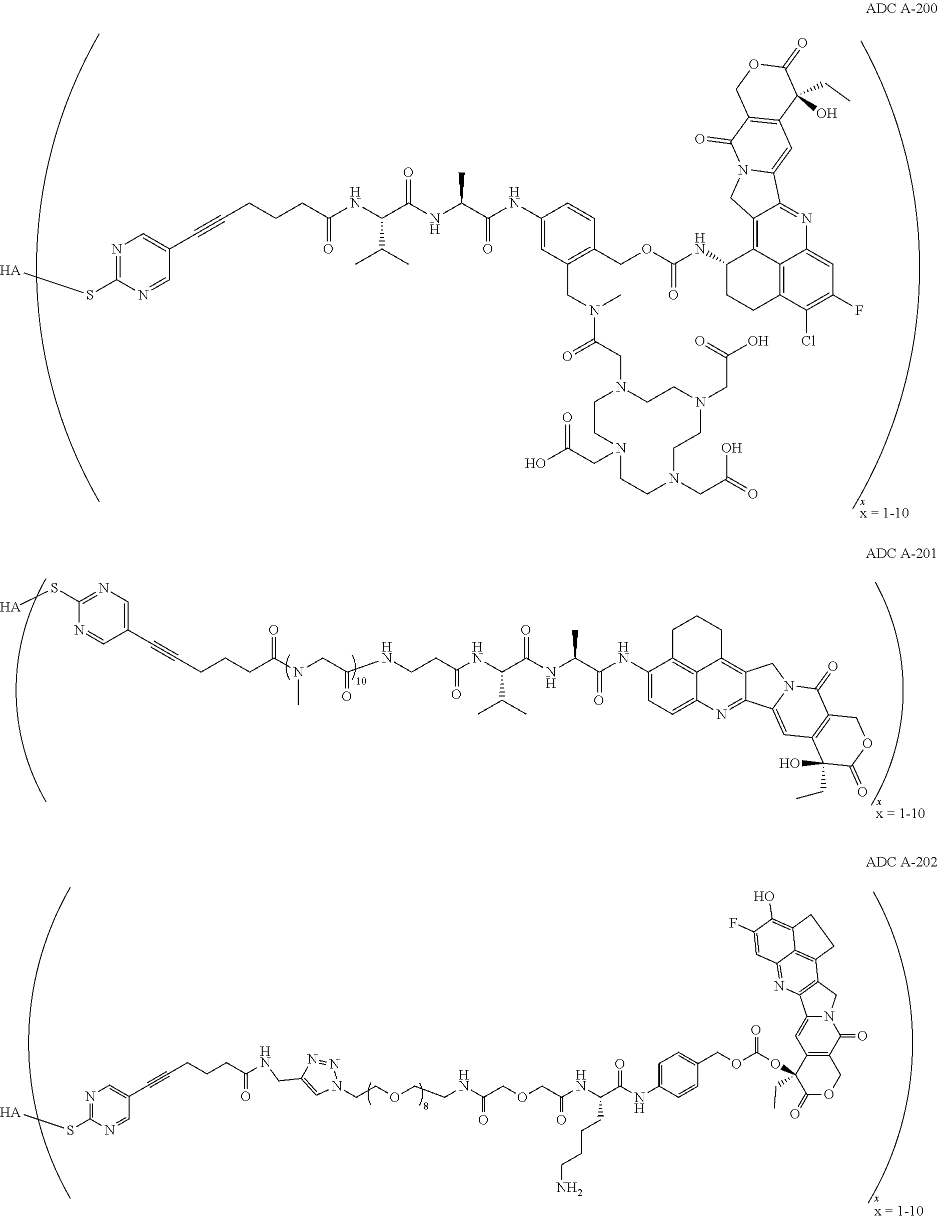

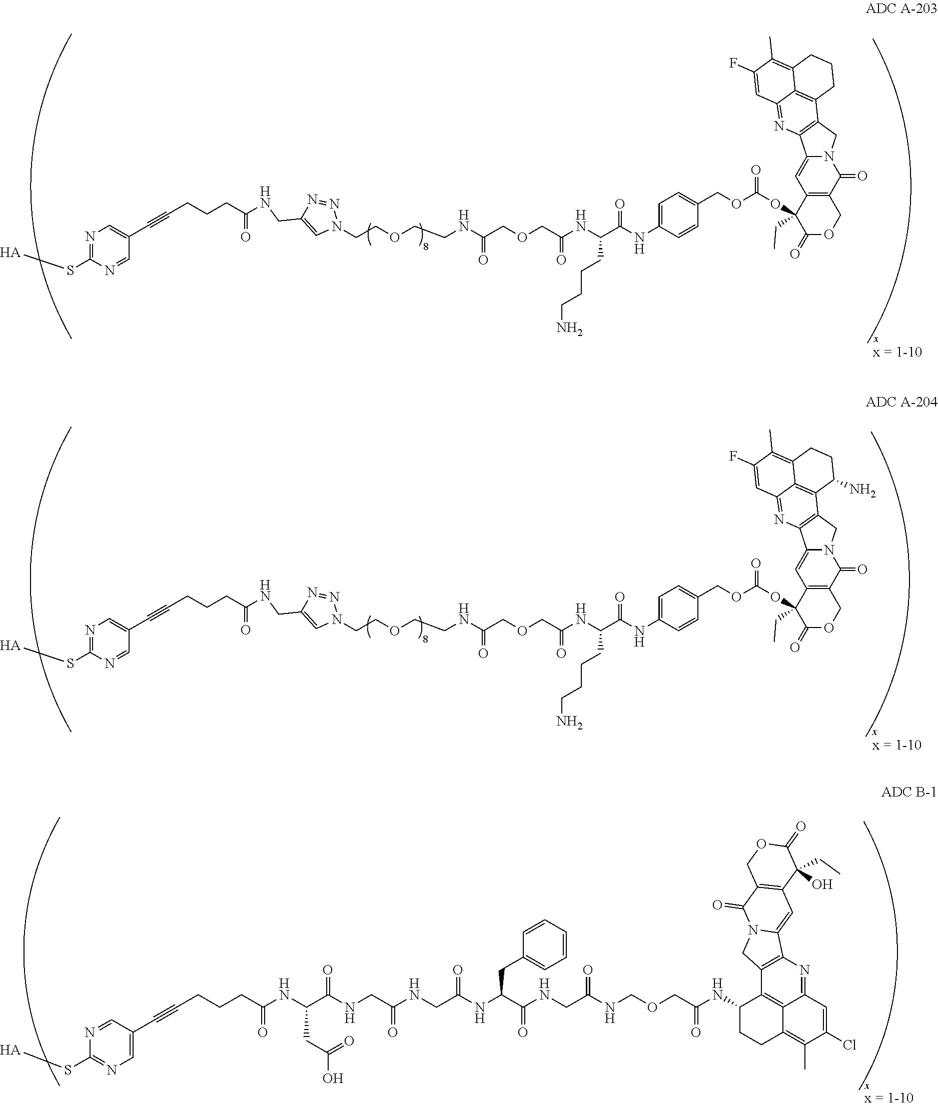

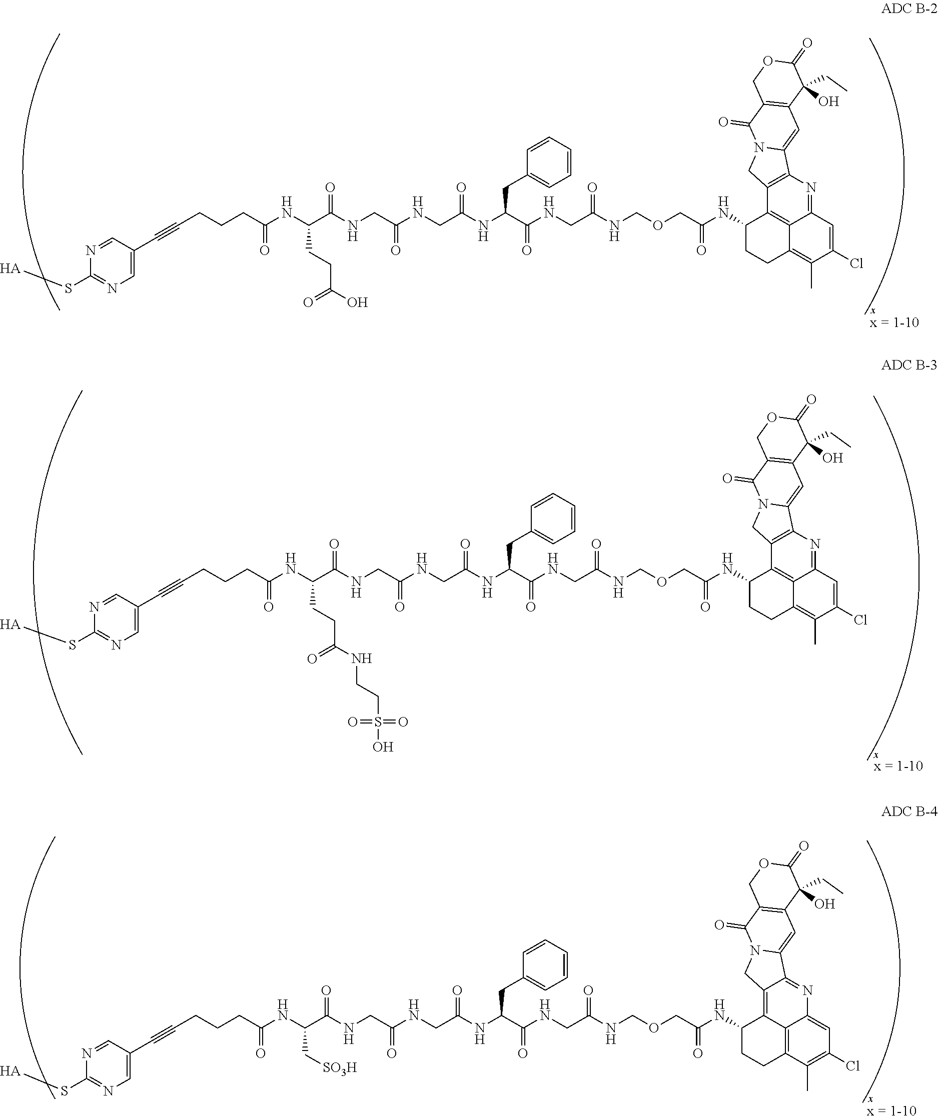

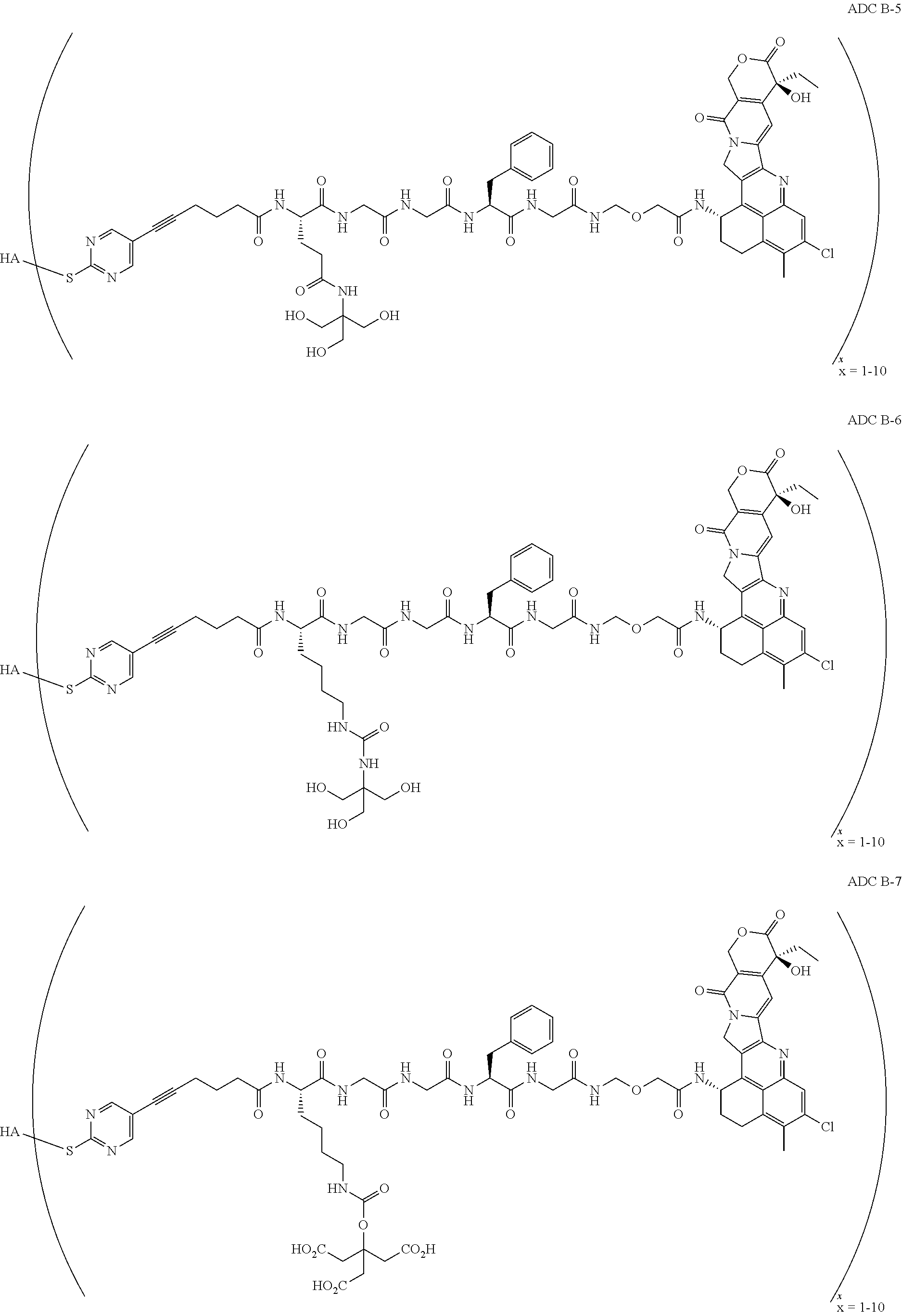

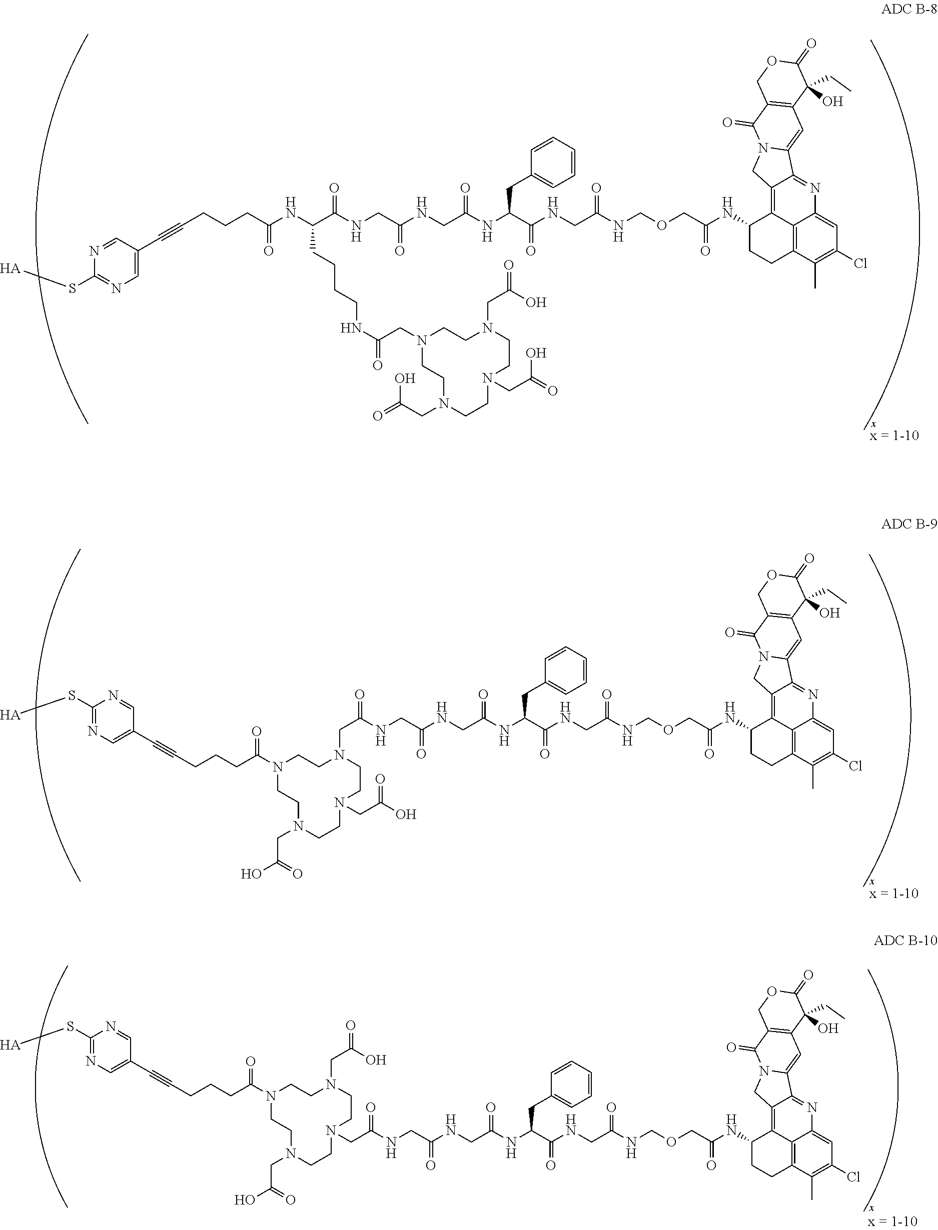

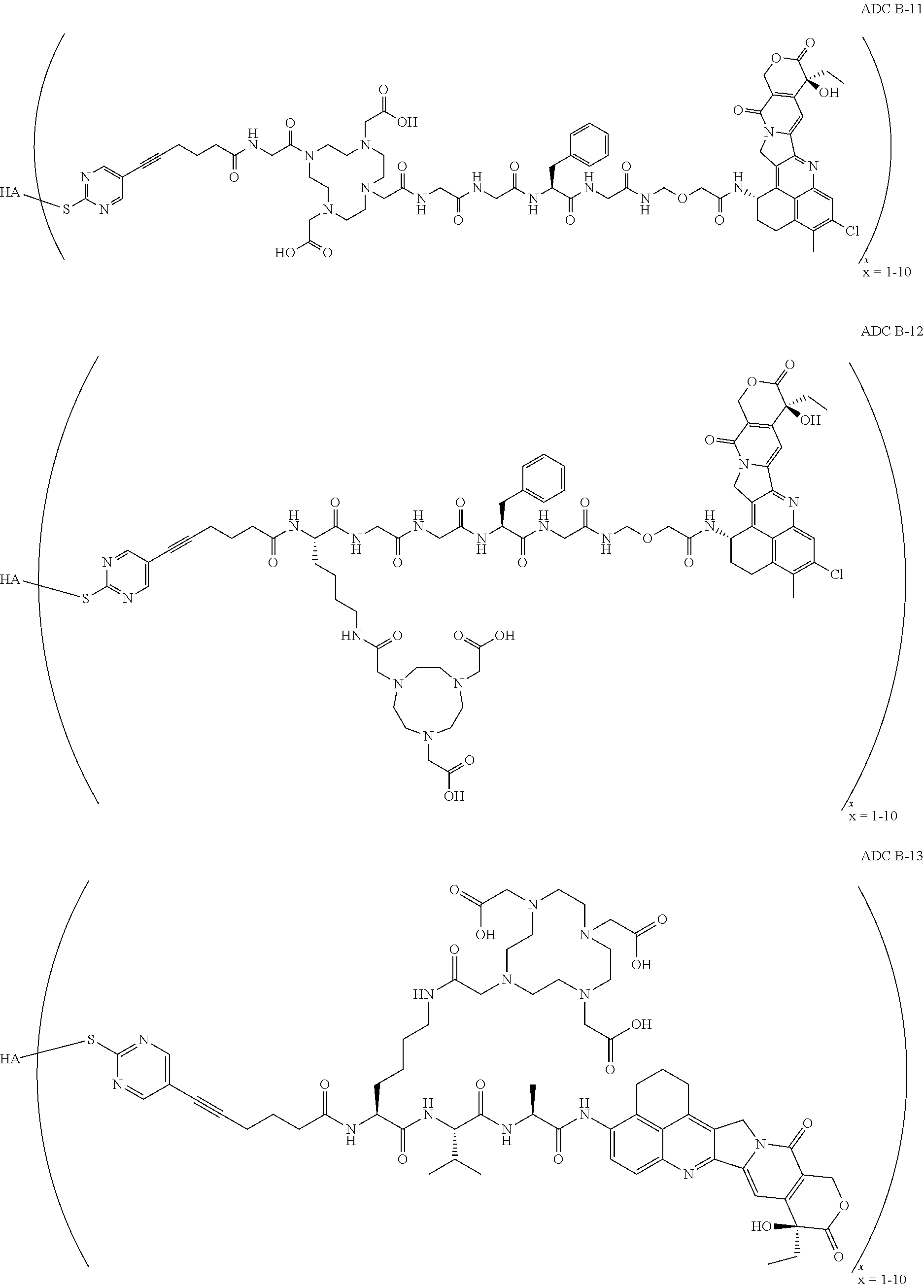

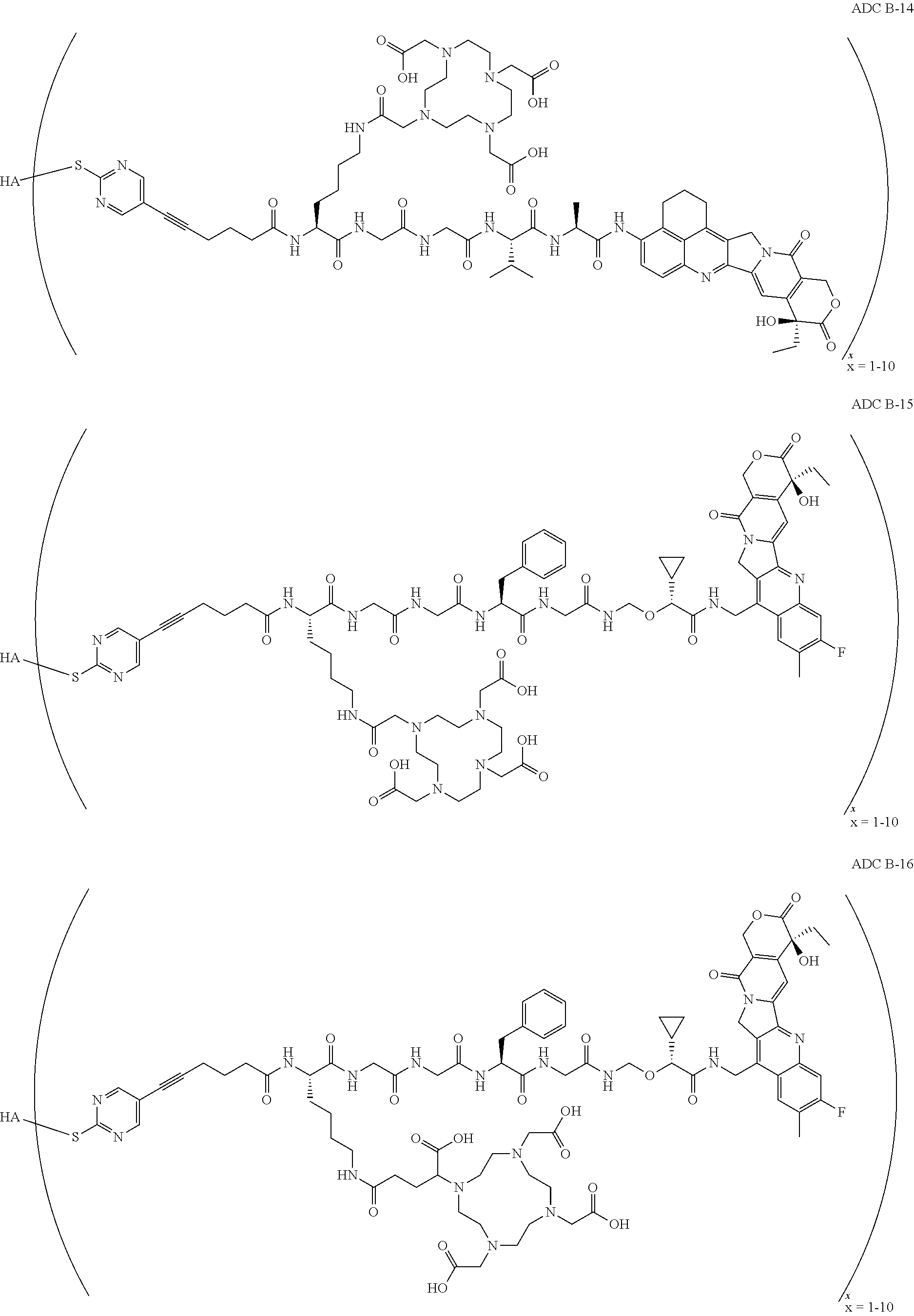

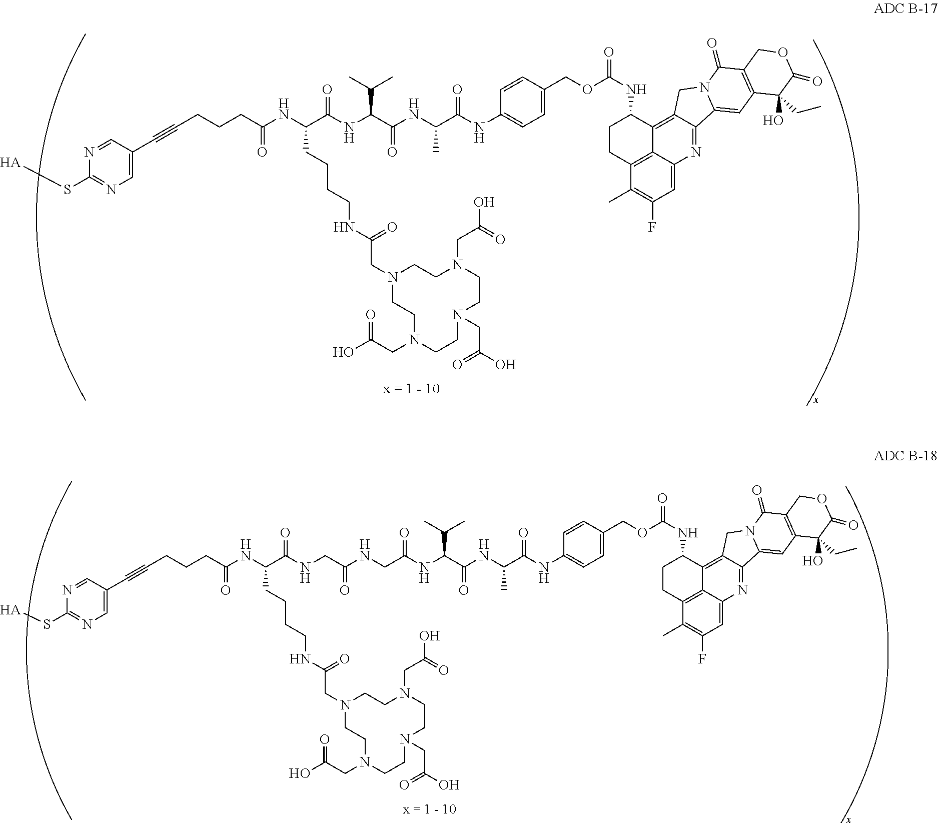

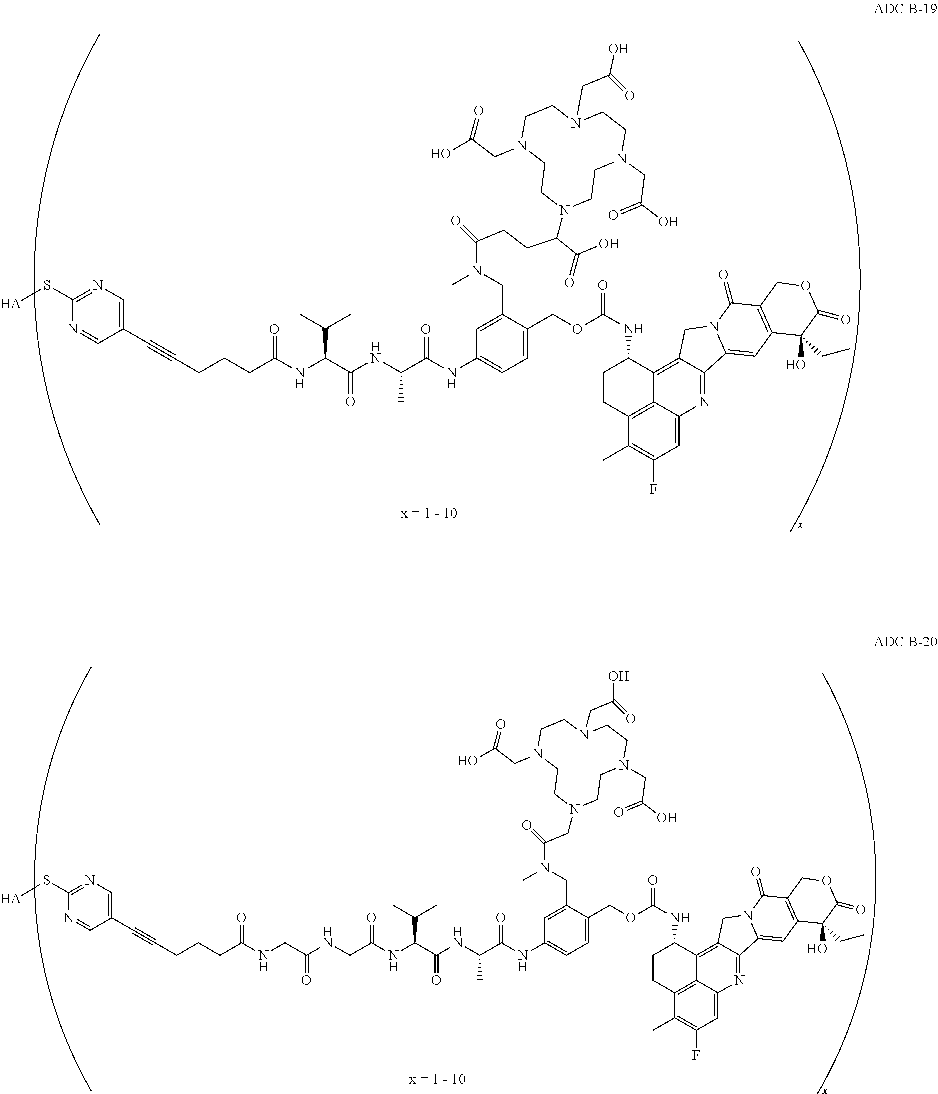

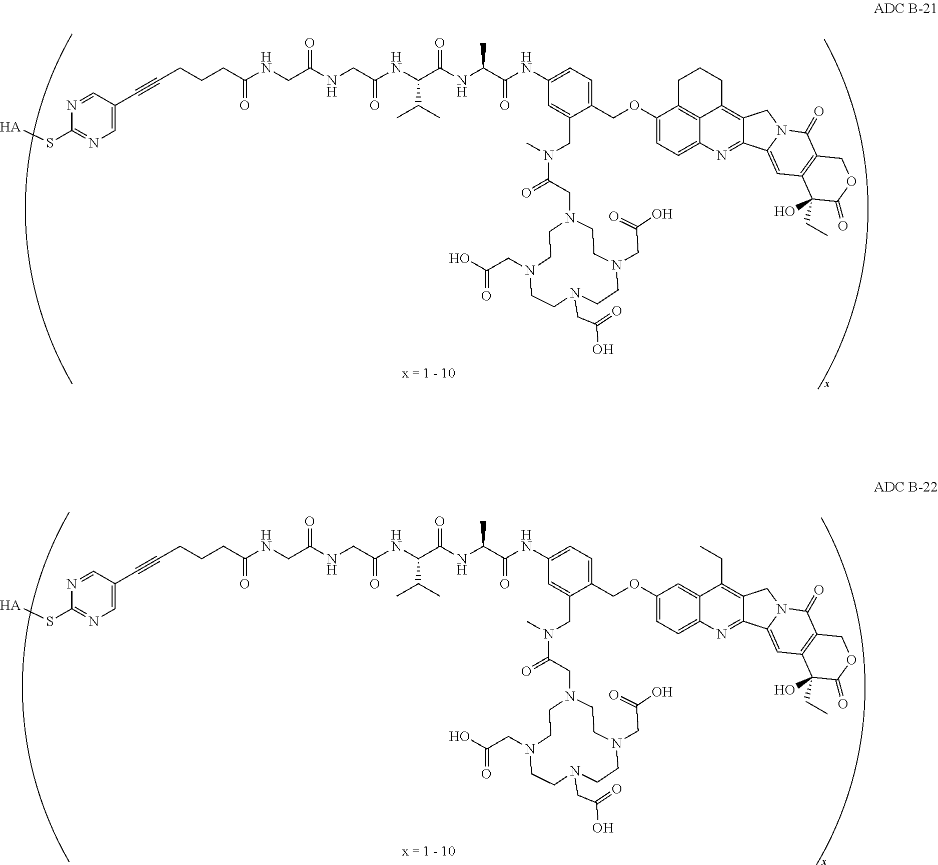

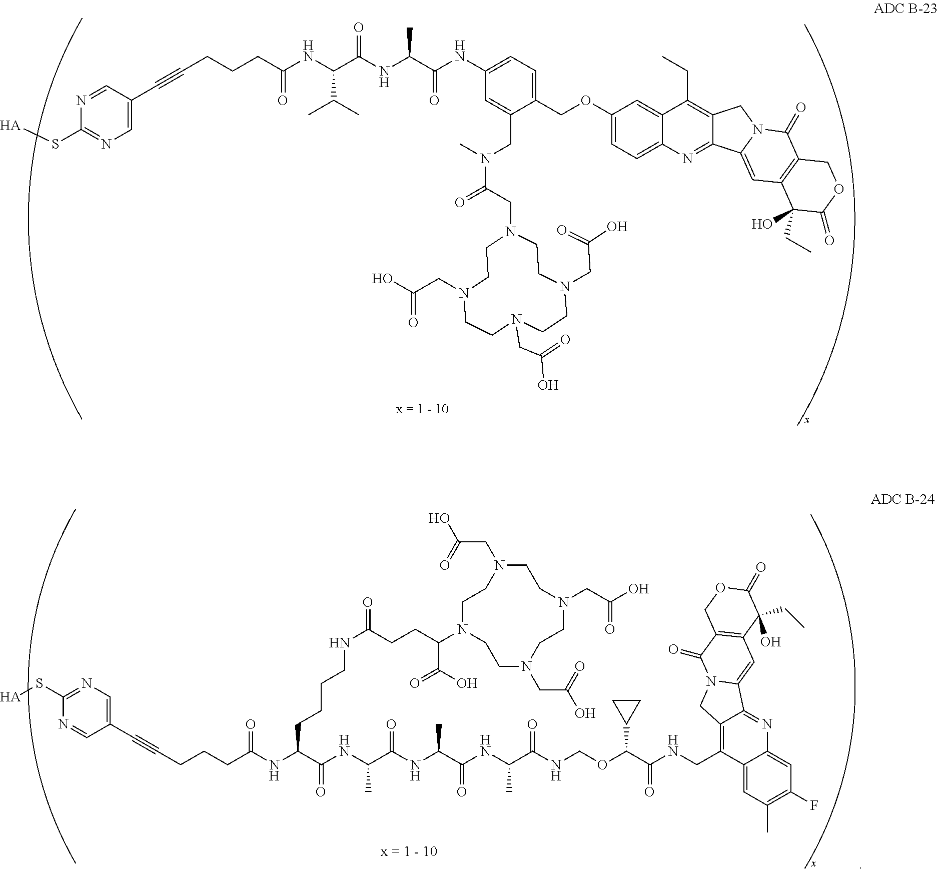

wherein, HA in each antibody-drug conjugate is an antibody or antigen-binding fragment thereof; n is selected from an integer of 1 to 20, such as an integer of 1 to 12, 3 to 12, 5 to 15, 5 to 10, such as 1, 2, 3, 4, 5, 6, 7, 8, 9, 10, 11, 12, 13, 14, 15, 16, 17, 18, 19, or 20;
wherein,

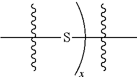 [SMILES: *S*]

represents a specific linkage between a sulfhydryl group in the antibody or antigen-binding fragment thereof and the M fragment.

In some embodiments, the antibody or antigen-binding fragment thereof in each antibody-drug conjugate is as defined above.

In some embodiments, x in the conjugate represented by Ab-[M-L-E-D]ₓ is 1 to 10, such as 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10 or 9 to 10.

In some embodiments, x in the conjugate represented by Ab-[M-L-E-D]ₓ is 1, 2, 3, 4, 5, 6, 7, 8, 9 or 10.

In some embodiments, the conjugate described in the present invention is an antibody-drug conjugate (ADC).

The conjugate of the present invention is optionally substituted with one or more suitable substituents.

The present application further provides an antibody-drug conjugate represented by the formula Ab-[L′-D]ₓ, wherein:
Ab represents an antibody or antigen-binding fragment thereof;
D represents a cytotoxic drug fragment;
L′ represents a linker connecting Ab and D;
x is an integer from 1 to 10;
the antibody or antigen-binding fragment thereof together with the compound selected from the group consisting of the aforementioned A-1 to A-204, or B-1 to B-24 form the antibody-drug conjugate.

In certain embodiments, the antibody comprises a heavy chain amino acid sequence as set forth in SEQ ID NO: 37, and a light chain amino acid sequence as set forth in SEQ ID NO: 38.

In certain embodiments, the antibody is Trastuzumab.

In certain embodiments, the antibody in the antibody-drug conjugate is conjugated to 1 to 8 (e.g., 1, 2, 3, 4, 5, 6, 7, 8; preferably 5 to 8) structures as shown below:

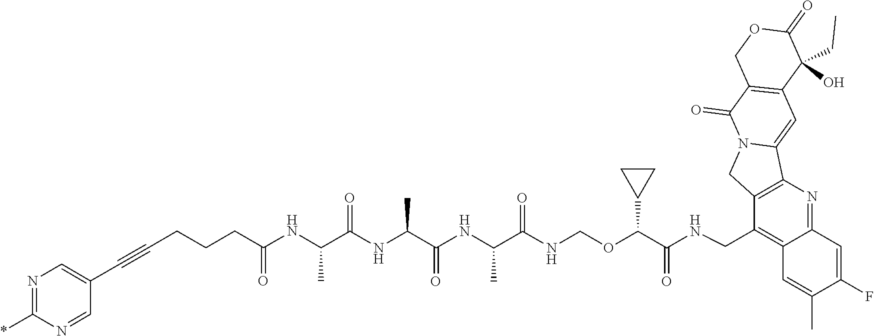 [SMILES: *c1ncc(C#CCCCC(=O)N[C@@H](C)C(=O)N[C@@H](C)C(=O)N[C@@H](C)C(=O)NCO[C@@H](C(=O)NCc2c3c(nc4cc(F)c(C)cc24)-c2cc4c(c(=O)n2C3)COC(=O)[C@]4(O)CC)C2CC2)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

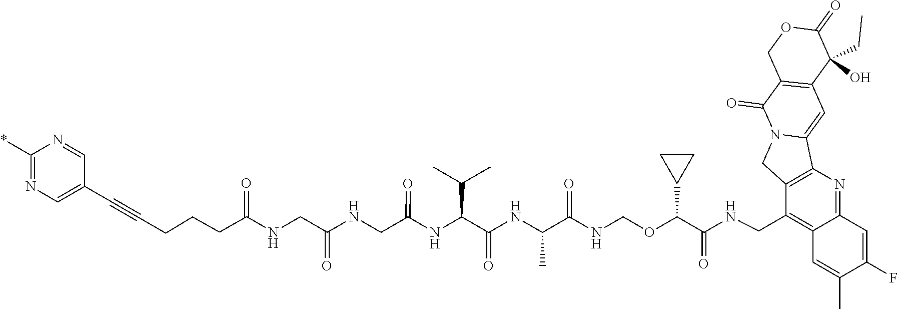 [SMILES: *c1ncc(C#CCCCC(=O)NCC(=O)NCC(=O)N[C@H](C(=O)N[C@@H](C)C(=O)NCO[C@@H](C(=O)NCc2c3c(nc4cc(F)c(C)cc24)-c2cc4c(c(=O)n2C3)COC(=O)[C@]4(O)CC)C2CC2)C(C)C)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

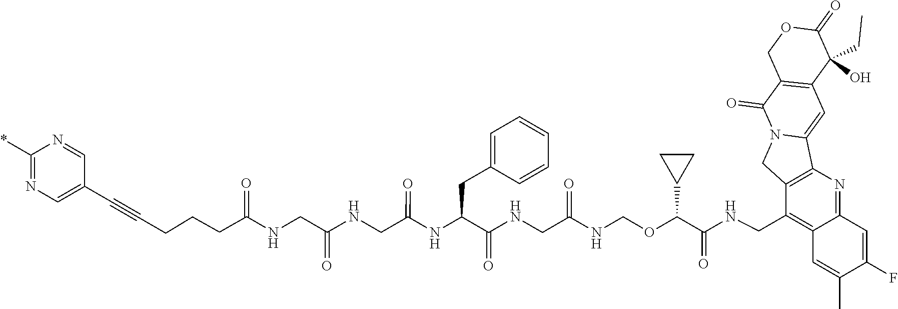 [SMILES: *c1ncc(C#CCCCC(=O)NCC(=O)NCC(=O)N[C@@H](Cc2ccccc2)C(=O)NCC(=O)NCO[C@@H](C(=O)NCc2c3c(nc4cc(F)c(C)cc24)-c2cc4c(c(=O)n2C3)COC(=O)[C@]4(O)CC)C2CC2)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

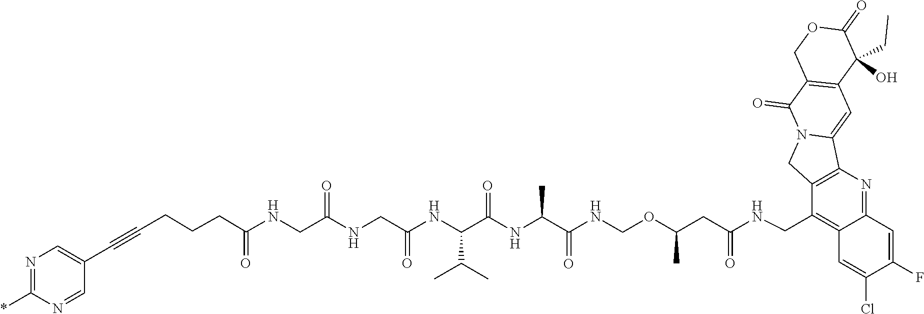 [SMILES: *c1ncc(C#CCCCC(=O)NCC(=O)NCC(=O)N[C@H](C(=O)N[C@@H](C)C(=O)NCO[C@H](C)CC(=O)NCc2c3c(nc4cc(F)c(Cl)cc24)-c2cc4c(c(=O)n2C3)COC(=O)[C@]4(O)CC)C(C)C)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

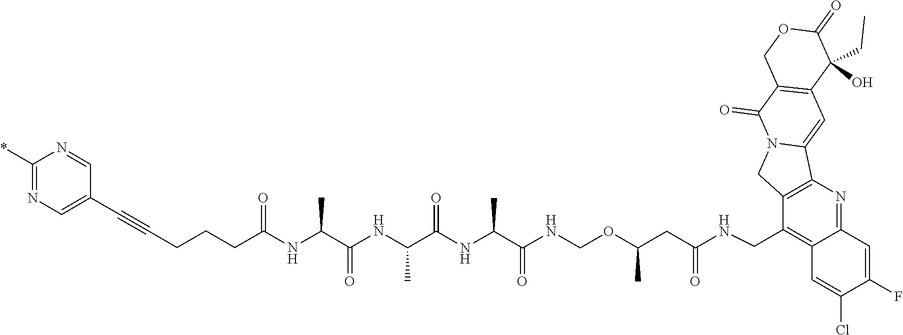 [SMILES: *c1ncc(C#CCCCC(=O)N[C@@H](C)C(=O)N[C@@H](C)C(=O)N[C@@H](C)C(=O)NCO[C@H](C)CC(=O)NCc2c3c(nc4cc(F)c(Cl)cc24)-c2cc4c(c(=O)n2C3)COC(=O)[C@]4(O)CC)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

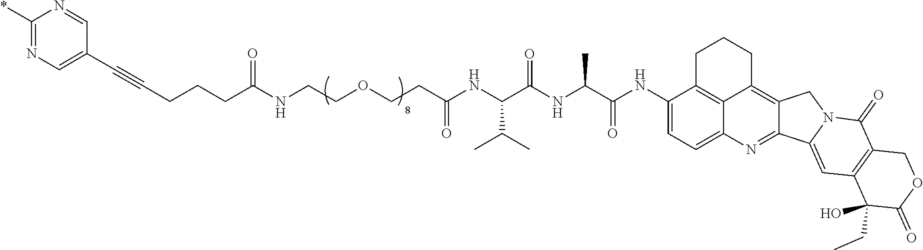

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

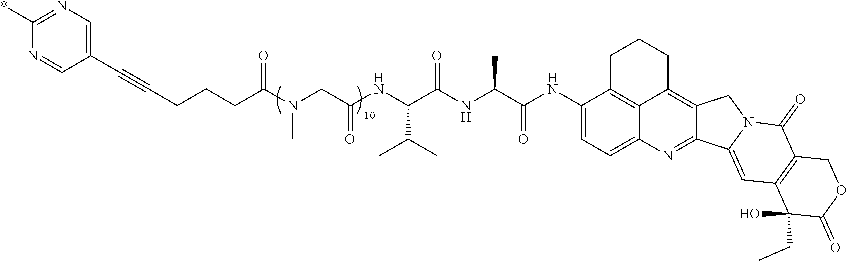

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

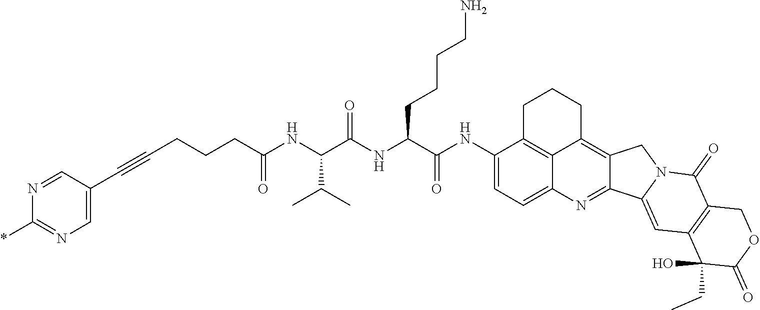 [SMILES: *c1ncc(C#CCCCC(=O)N[C@H](C(=O)N[C@@H](CCCCN)C(=O)Nc2ccc3nc4c(c5c3c2CCC5)Cn2c-4cc3c(c2=O)COC(=O)[C@]3(O)CC)C(C)C)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

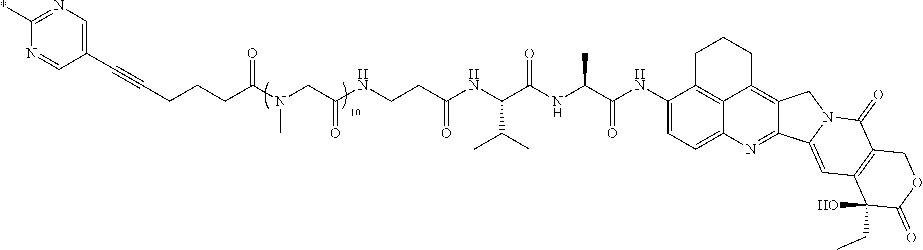

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

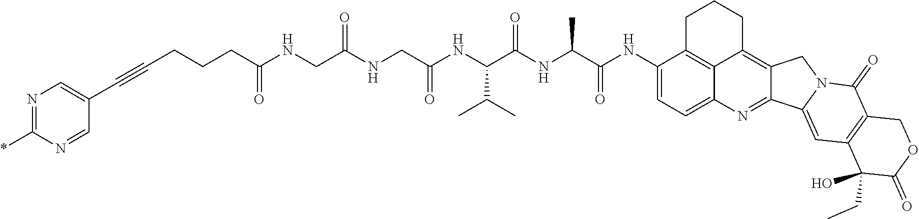 [SMILES: *c1ncc(C#CCCCC(=O)NCC(=O)NCC(=O)N[C@H](C(=O)N[C@@H](C)C(=O)Nc2ccc3nc4c(c5c3c2CCC5)Cn2c-4cc3c(c2=O)COC(=O)[C@]3(O)CC)C(C)C)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

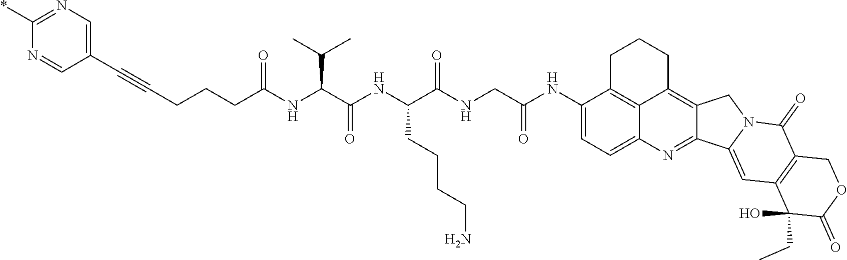 [SMILES: *c1ncc(C#CCCCC(=O)N[C@H](C(=O)N[C@@H](CCCCN)C(=O)NCC(=O)Nc2ccc3nc4c(c5c3c2CCC5)Cn2c-4cc3c(c2=O)COC(=O)[C@]3(O)CC)C(C)C)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

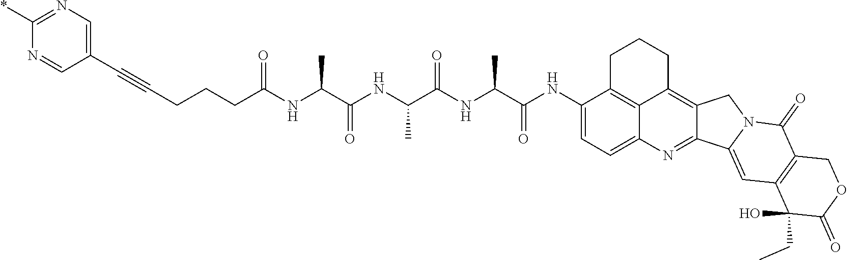 [SMILES: *c1ncc(C#CCCCC(=O)N[C@@H](C)C(=O)N[C@@H](C)C(=O)N[C@@H](C)C(=O)Nc2ccc3nc4c(c5c3c2CCC5)Cn2c-4cc3c(c2=O)COC(=O)[C@]3(O)CC)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

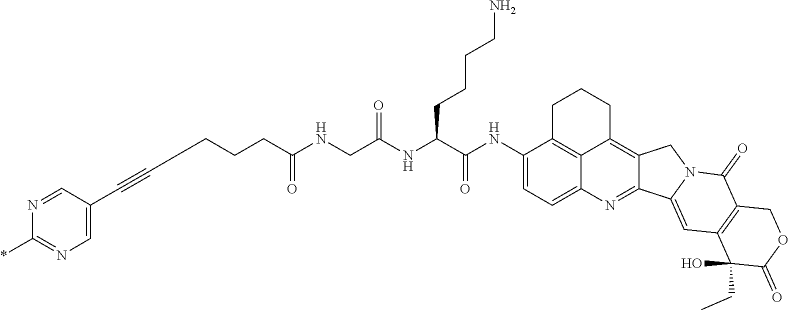 [SMILES: *c1ncc(C#CCCCCC(=O)NCC(=O)N[C@@H](CCCCN)C(=O)Nc2ccc3nc4c(c5c3c2CCC5)Cn2c-4cc3c(c2=O)COC(=O)[C@]3(O)CC)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

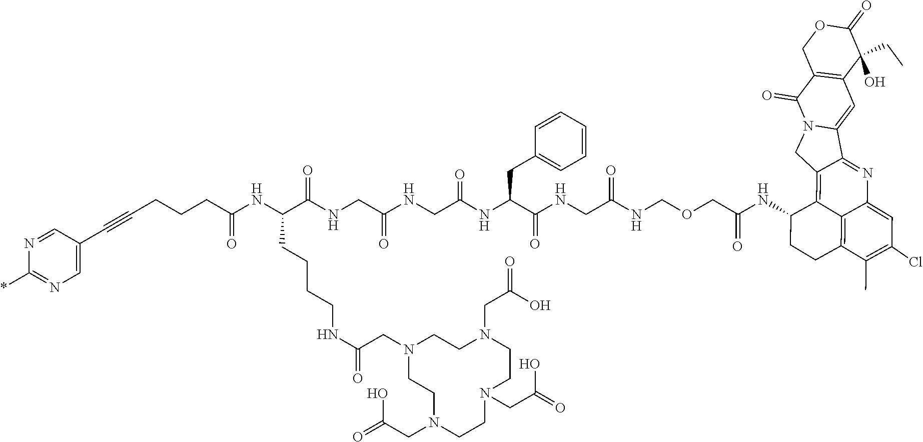 [SMILES: *c1ncc(C#CCCCC(=O)N[C@@H](CCCCNC(=O)CN2CCN(CC(=O)O)CCN(CC(=O)O)CCN(CC(=O)O)CC2)C(=O)NCC(=O)NCC(=O)N[C@@H](Cc2ccccc2)C(=O)NCC(=O)NCOCC(=O)N[C@H]2CCc3c(C)c(Cl)cc4nc5c(c2c34)Cn2c-5cc3c(c2=O)COC(=O)[C@]3(O)CC)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

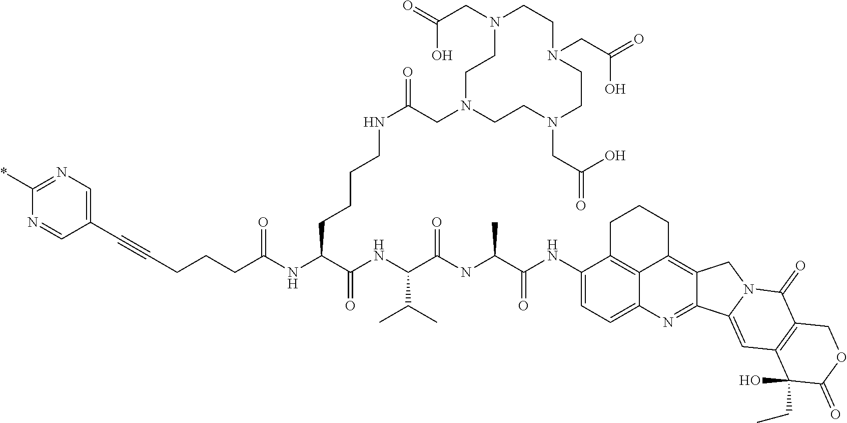

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

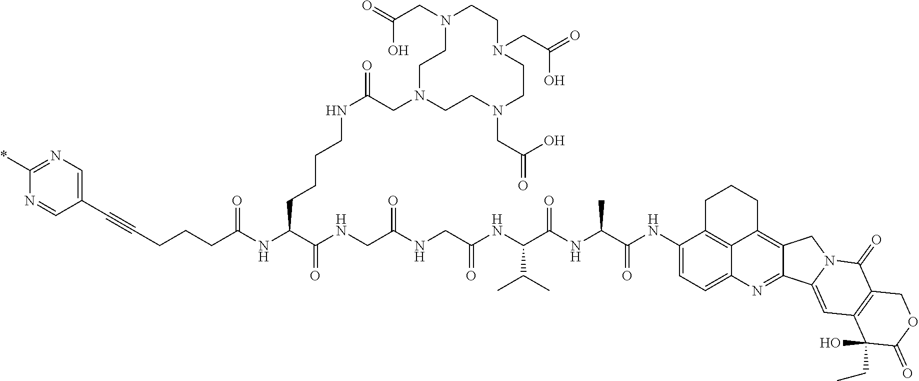 [SMILES: *c1ncc(C#CCCCC(=O)N[C@@H](CCCCNC(=O)CN2CCN(CC(=O)O)CCN(CC(=O)O)CCN(CC(=O)O)CC2)C(=O)NCC(=O)NCC(=O)N[C@H](C(=O)N[C@@H](C)C(=O)Nc2ccc3nc4c(c5c3c2CCC5)Cn2c-4cc3c(c2=O)COC(=O)[C@]3(O)CC)C(C)C)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

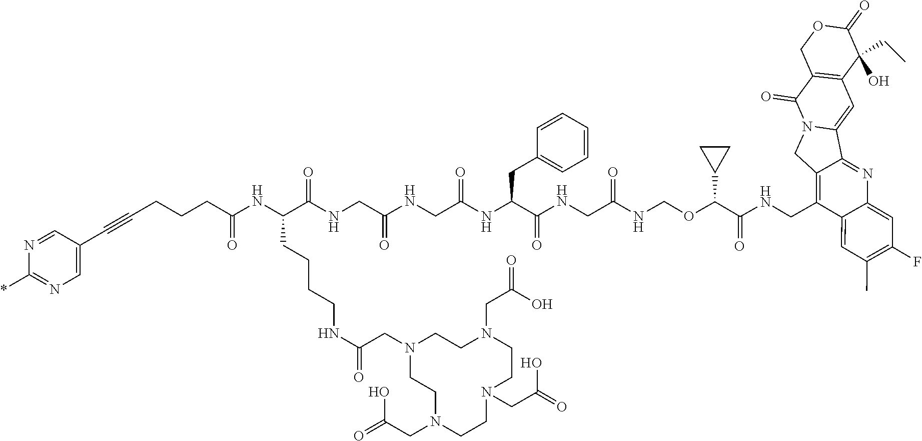 [SMILES: *c1ncc(C#CCCCC(=O)N[C@@H](CCCCNC(=O)CN2CCN(CC(=O)O)CCN(CC(=O)O)CCN(CC(=O)O)CC2)C(=O)NCC(=O)NCC(=O)N[C@@H](Cc2ccccc2)C(=O)NCC(=O)NCO[C@@H](C(=O)NCc2c3c(nc4cc(F)c(C)cc24)-c2cc4c(c(=O)n2C3)COC(=O)[C@]4(O)CC)C2CC2)cn1]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

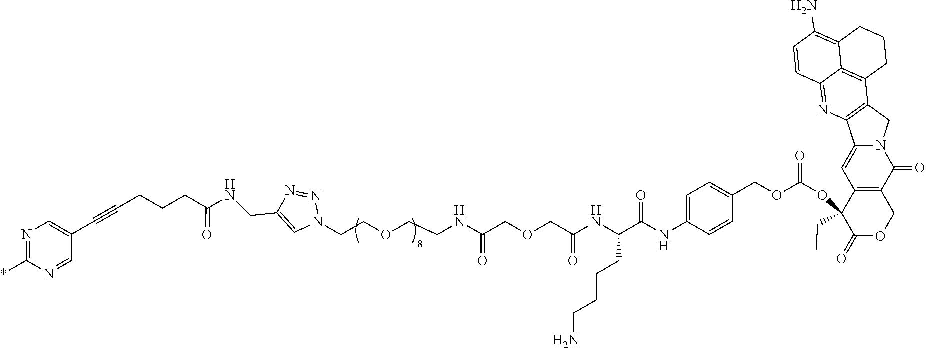

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

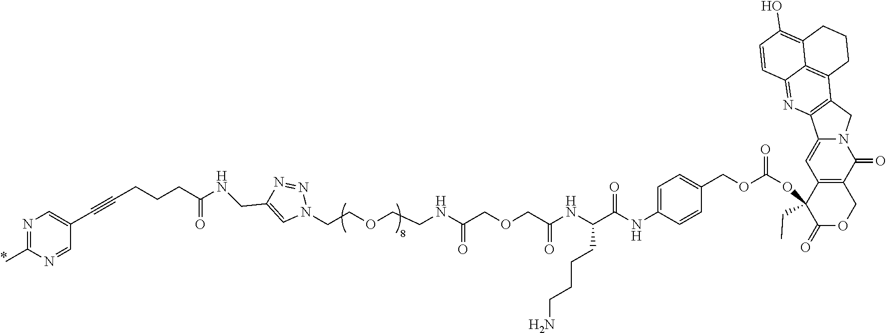

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

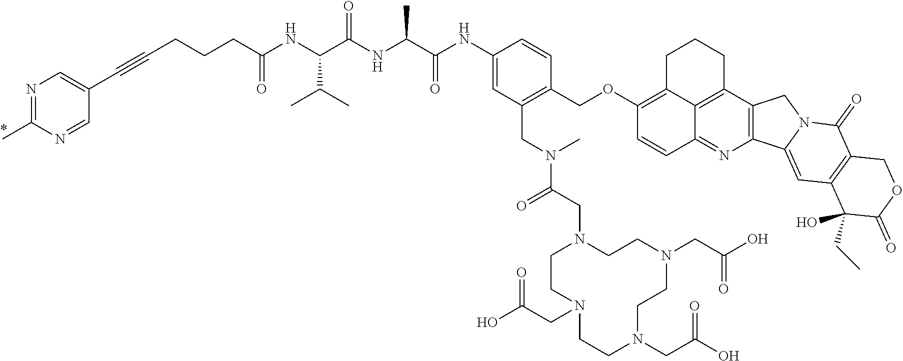 [SMILES: CC[C@@]1(O)C(=O)OCc2c1cc1n(c2=O)Cc2c-1nc1ccc(OCc3ccc(NC(=O)[C@H](C)NC(=O)[C@@H](NC(=O)CCCC#Cc4cnc(C)nc4)C(C)C)cc3CN(C)C(=O)CN3CCN(CC(=O)O)CCN(CC(=O)O)CCN(CC(=O)O)CC3)c3c1c2CCC3]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

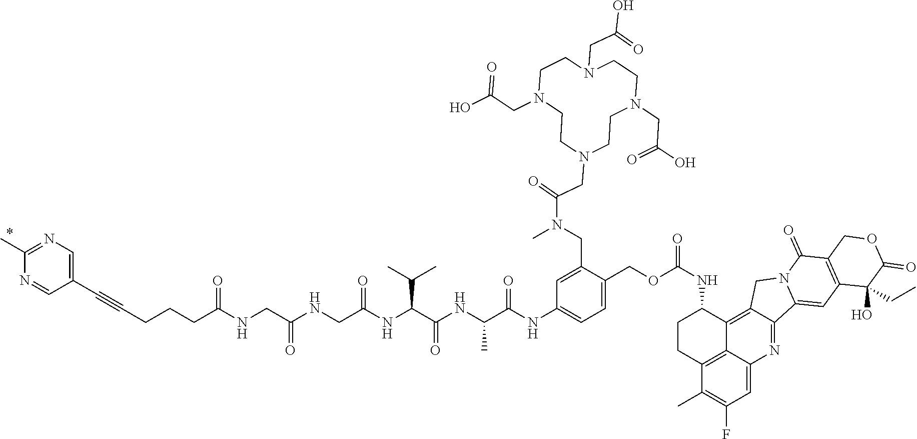 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CNC(=O)CNC(=O)CCCC#Cc2cnc(C)nc2)C(C)C)cc1CN(C)C(=O)CN1CCN(CC(=O)O)CCN(CC(=O)O)CCN(CC(=O)O)CC1)CC3]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

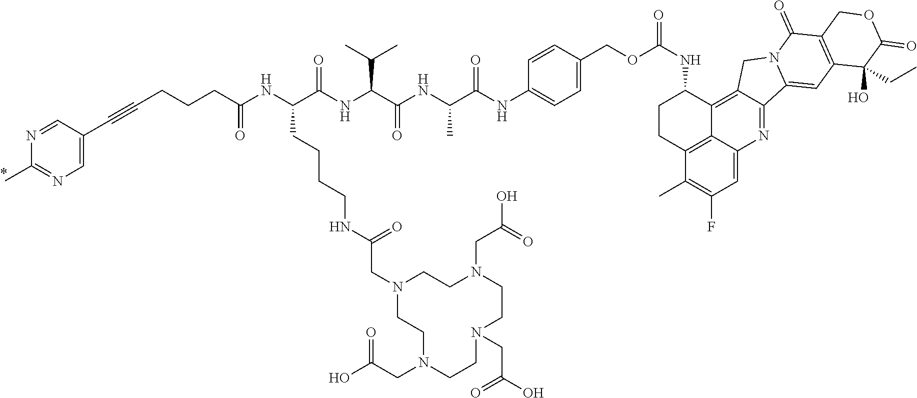 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCCCNC(=O)CN2CCN(CC(=O)O)CCN(CC(=O)O)CCN(CC(=O)O)CC2)NC(=O)CCCC#Cc2cnc(C)nc2)C(C)C)cc1)CC3]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

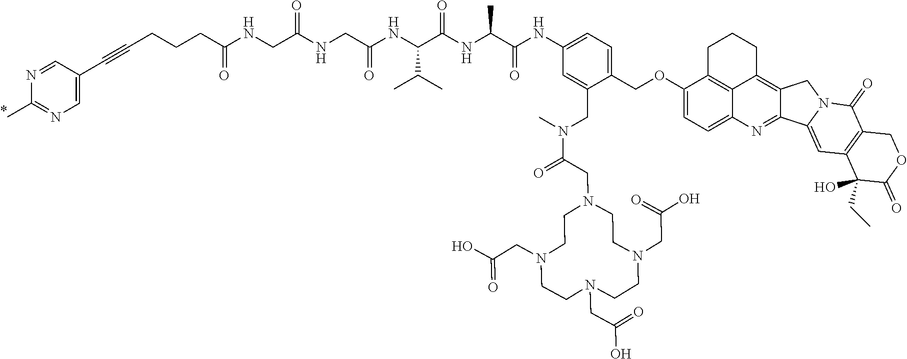 [SMILES: CC[C@@]1(O)C(=O)OCc2c1cc1n(c2=O)Cc2c-1nc1ccc(OCc3ccc(NC(=O)[C@H](C)NC(=O)[C@@H](NC(=O)CNC(=O)CNC(=O)CCCC#Cc4cnc(C)nc4)C(C)C)cc3CN(C)C(=O)CN3CCN(CC(=O)O)CCN(CC(=O)O)CCN(CC(=O)O)CC3)c3c1c2CCC3]

wherein * represents the connection point of the sulfhydryl group of the cysteine of the conjugated antibody.

In certain embodiments, the antibody in the antibody-drug conjugate is conjugated to 1 to 8 (e.g., 1, 2, 3, 4, 5, 6, 7, 8; preferably 5 to 8) structures as shown below:

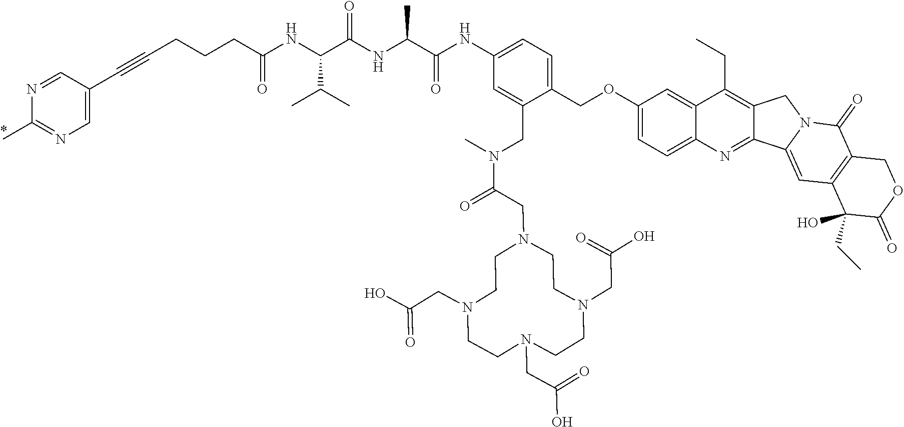 [SMILES: CCc1c2c(nc3ccc(OCc4ccc(NC(=O)[C@H](C)NC(=O)[C@@H](NC(=O)CCCC#Cc5cnc(C)nc5)C(C)C)cc4CN(C)C(=O)CN4CCN(CC(=O)O)CCN(CC(=O)O)CCN(CC(=O)O)CC4)cc13)-c1cc3c(c(=O)n1C2)COC(=O)[C@]3(O)CC]

wherein * represents the connection point of the sulfhydryl group of the cysteine of the coupled antibody.

In certain embodiments, the antibody in the antibody-drug conjugate is conjugated to 1 to 8 (e.g., 1, 2, 3, 4, 5, 6, 7, 8; preferably 5 to 8) structures as shown below:

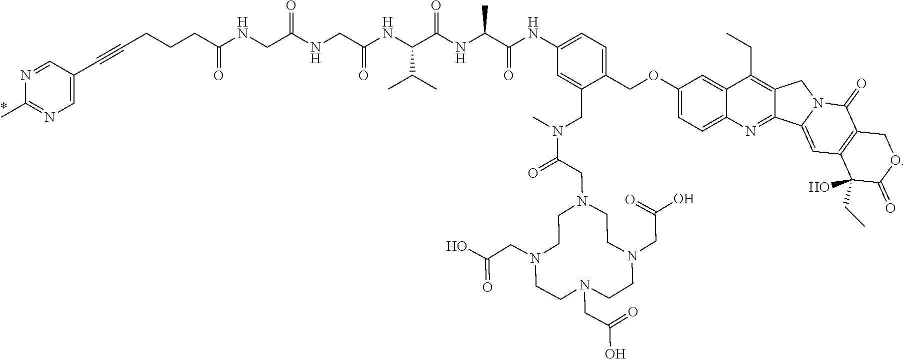 [SMILES: CCc1c2c(nc3ccc(OCc4ccc(NC(=O)[C@H](C)NC(=O)[C@@H](NC(=O)CNC(=O)CNC(=O)CCCC#Cc5cnc(C)nc5)C(C)C)cc4CN(C)C(=O)CN4CCN(CC(=O)O)CCN(CC(=O)O)CCN(CC(=O)O)CC4)cc13)-c1cc3c(c(=O)n1C2)COC(=O)[C@]3(O)CC]

### Intermediates

In some embodiments, the present application provides intermediate compounds having the following structures or salts, stereoisomers, tautomers or isotopically labeled compounds thereof:

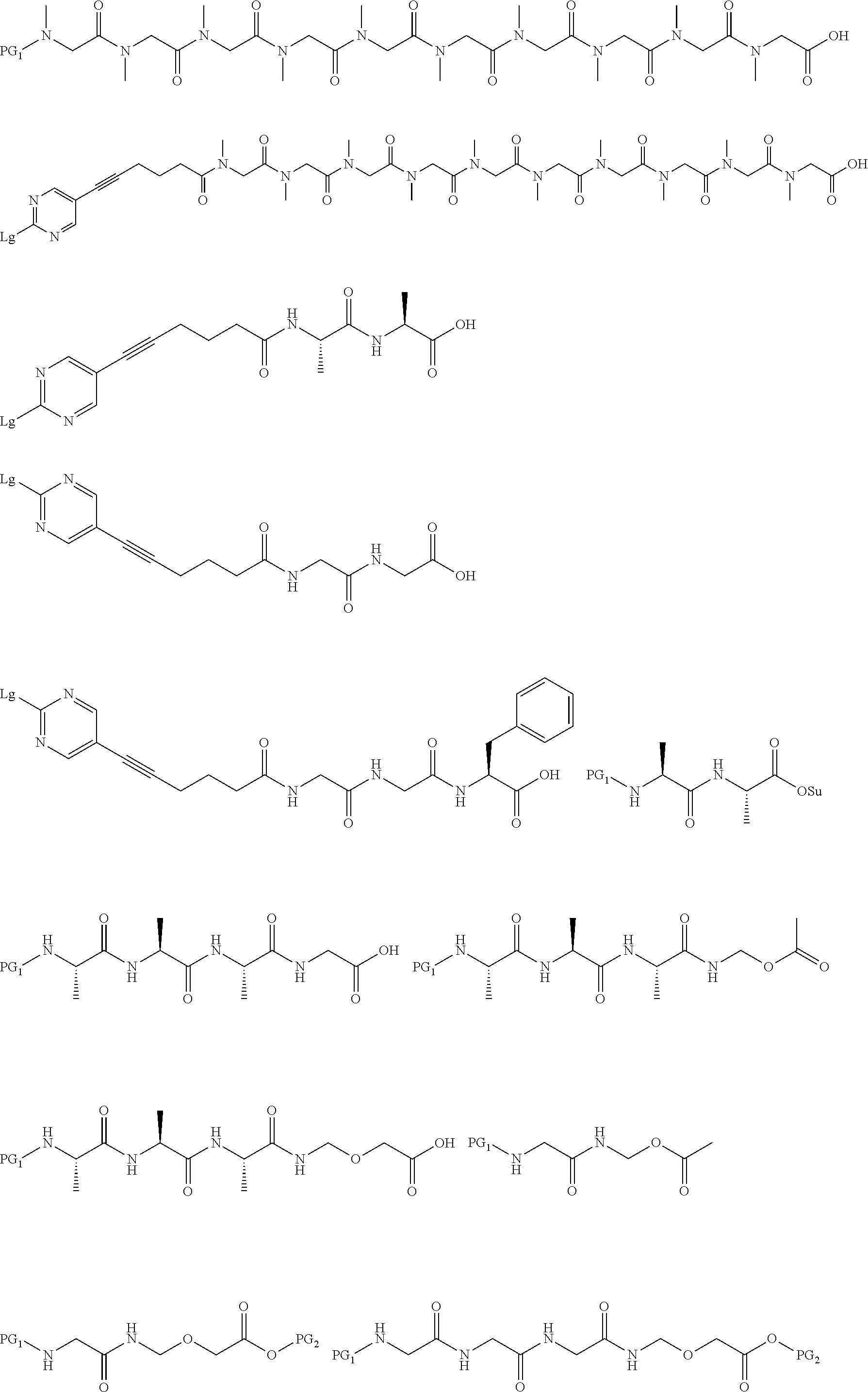

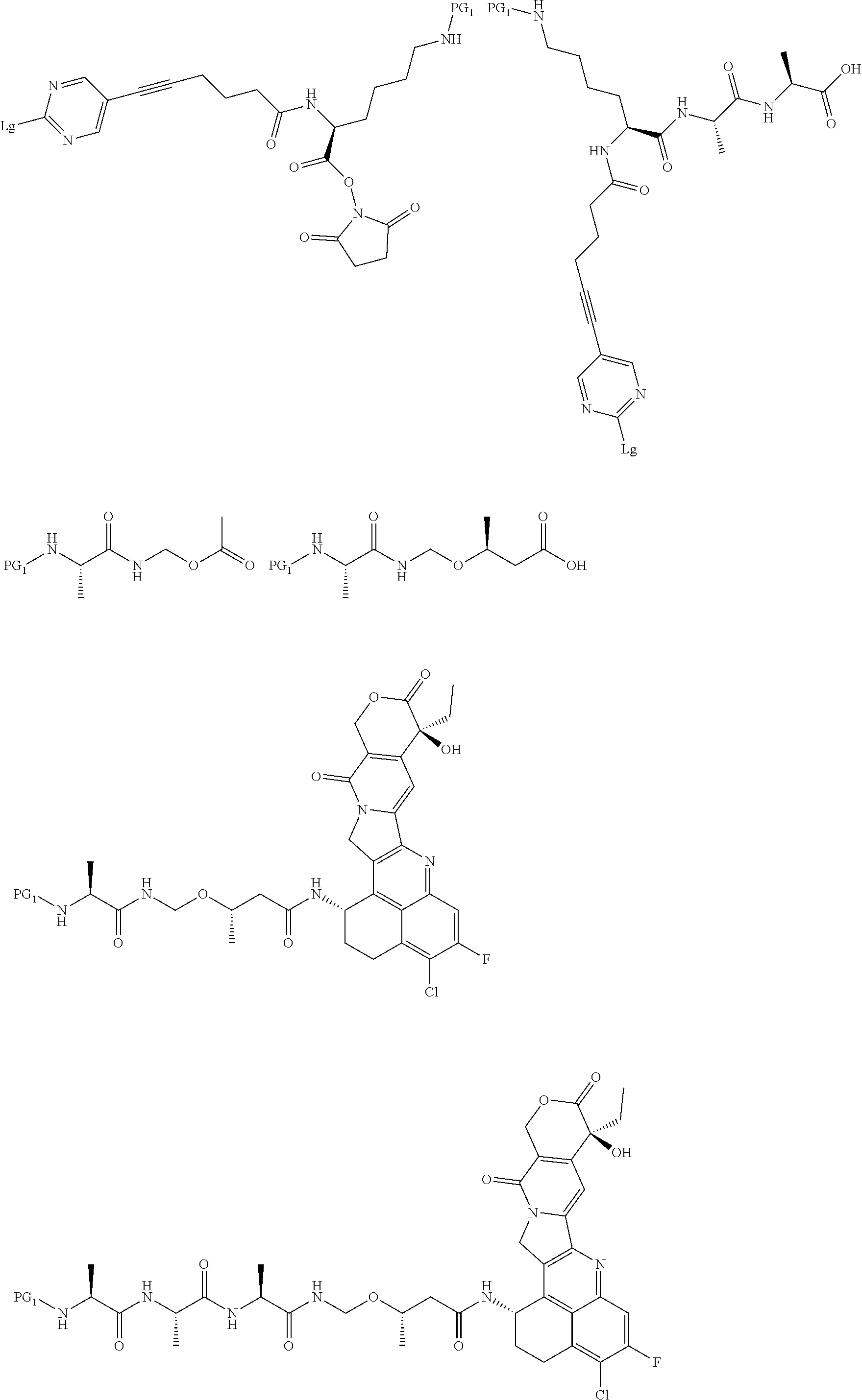

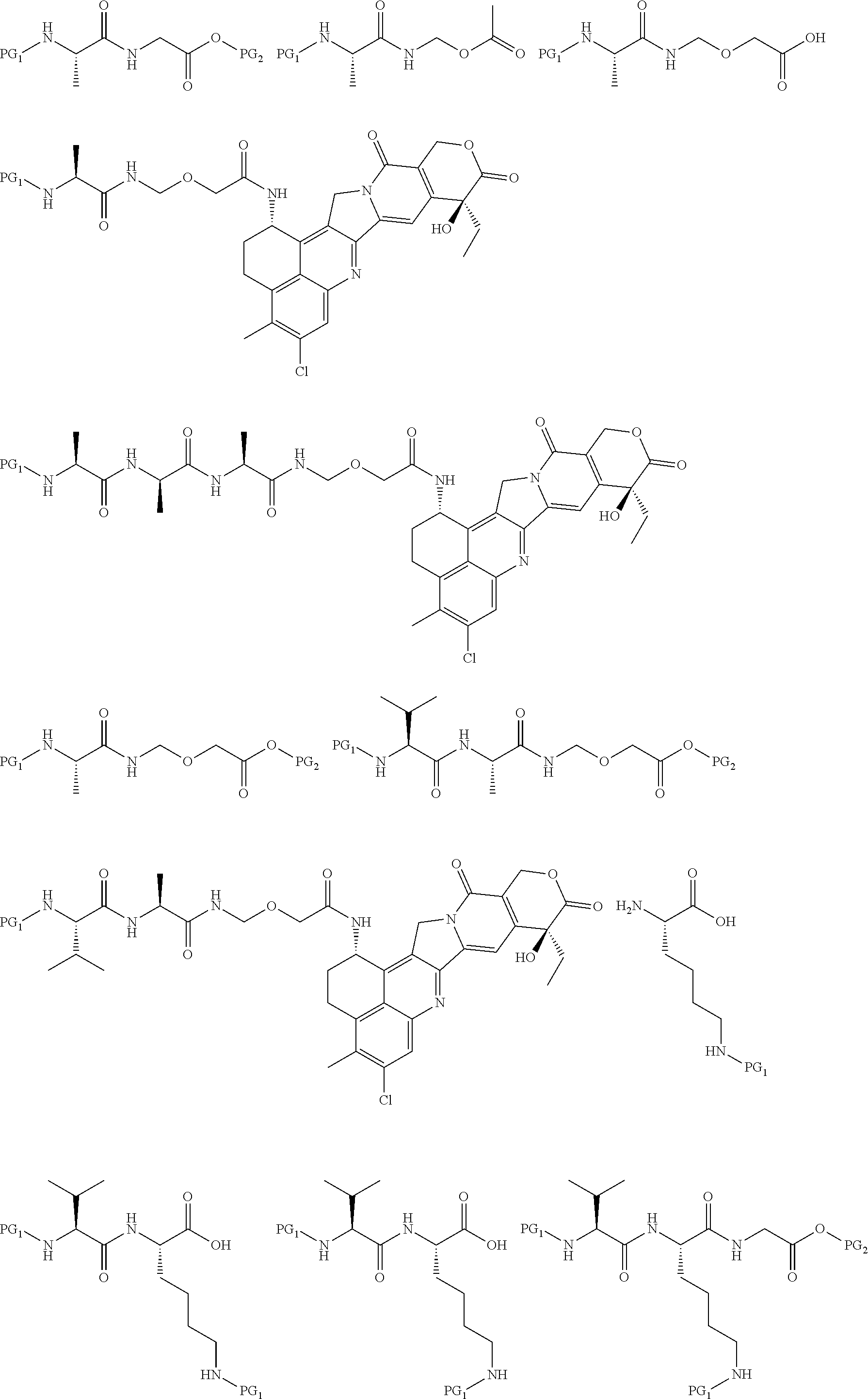

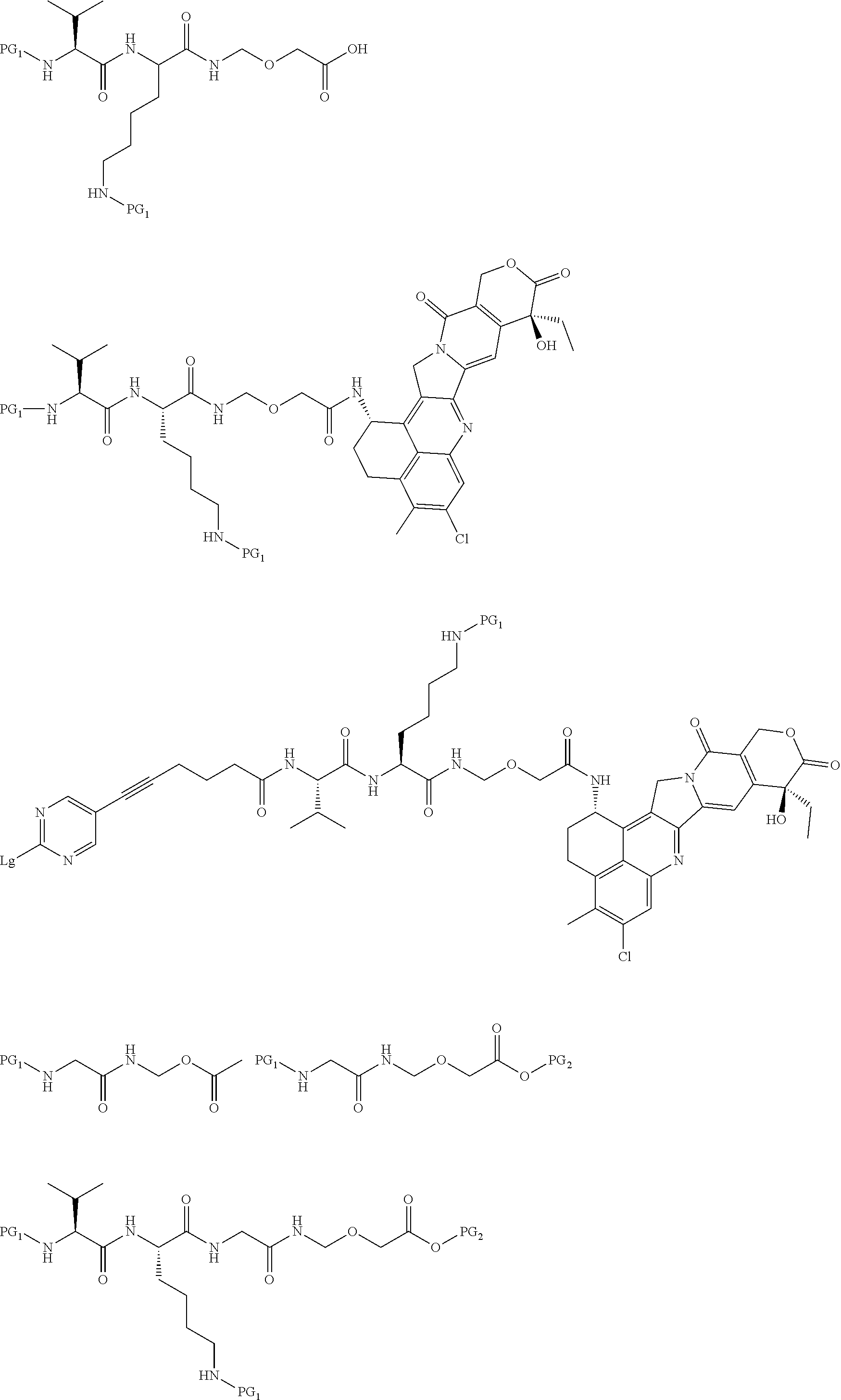

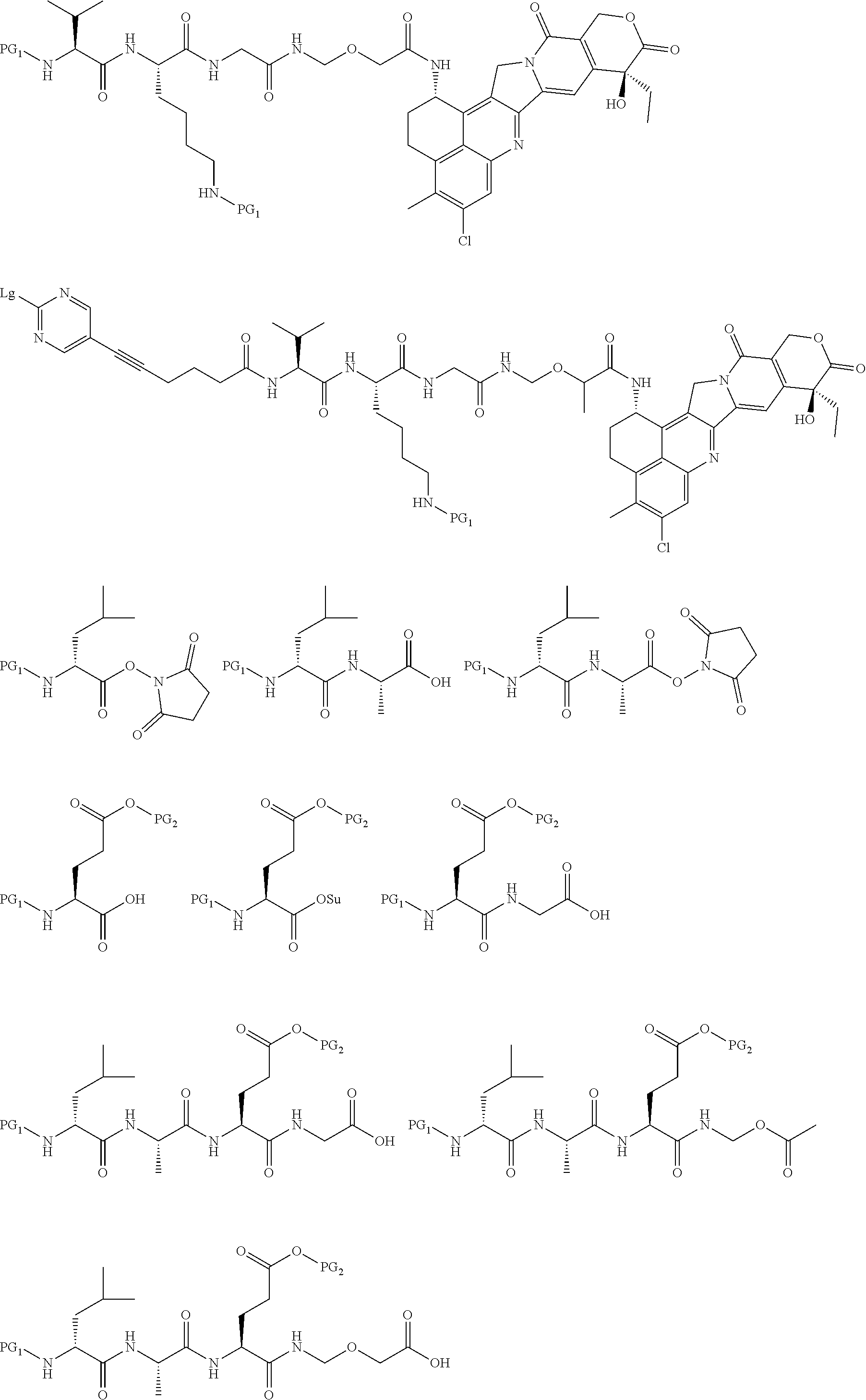

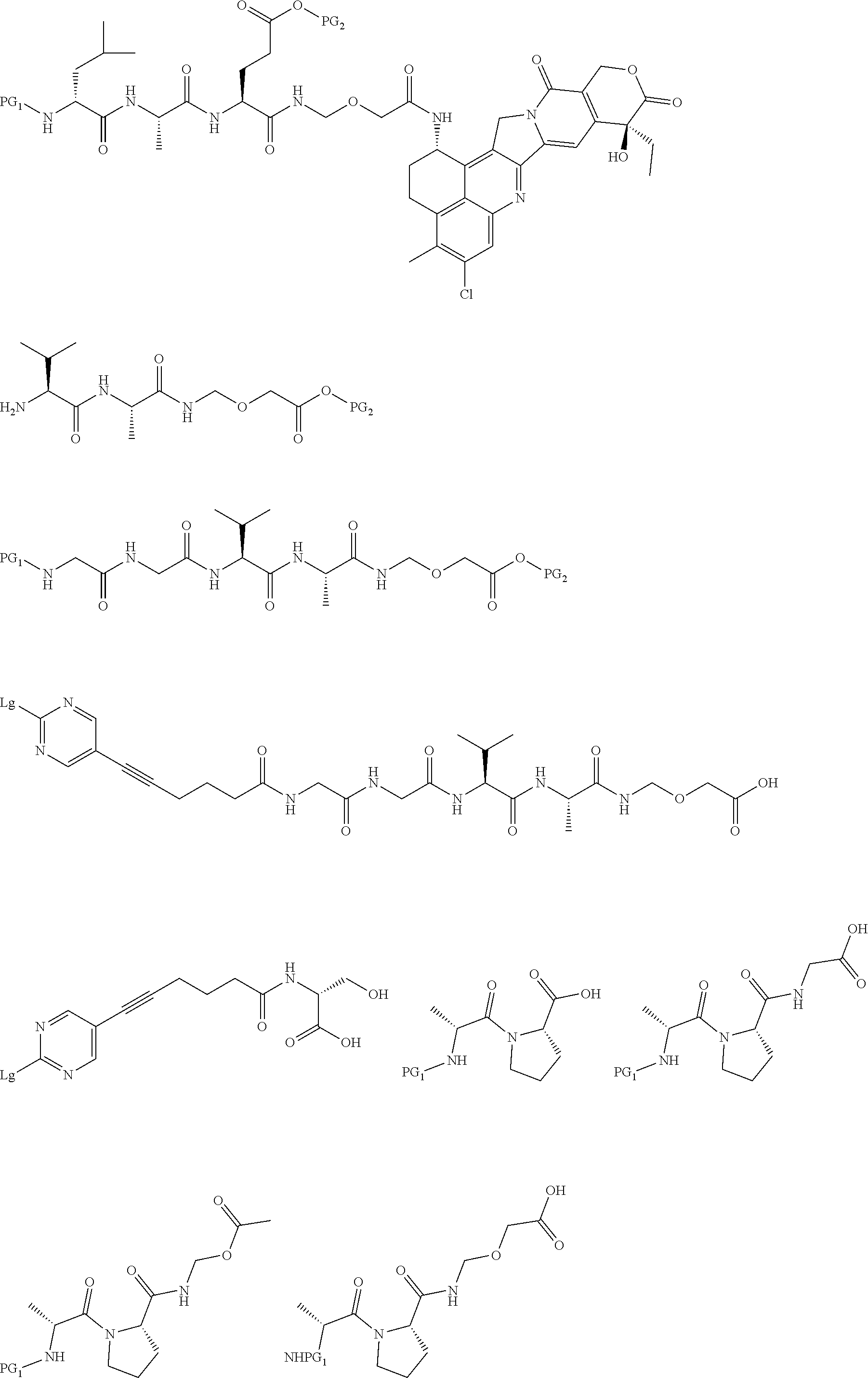

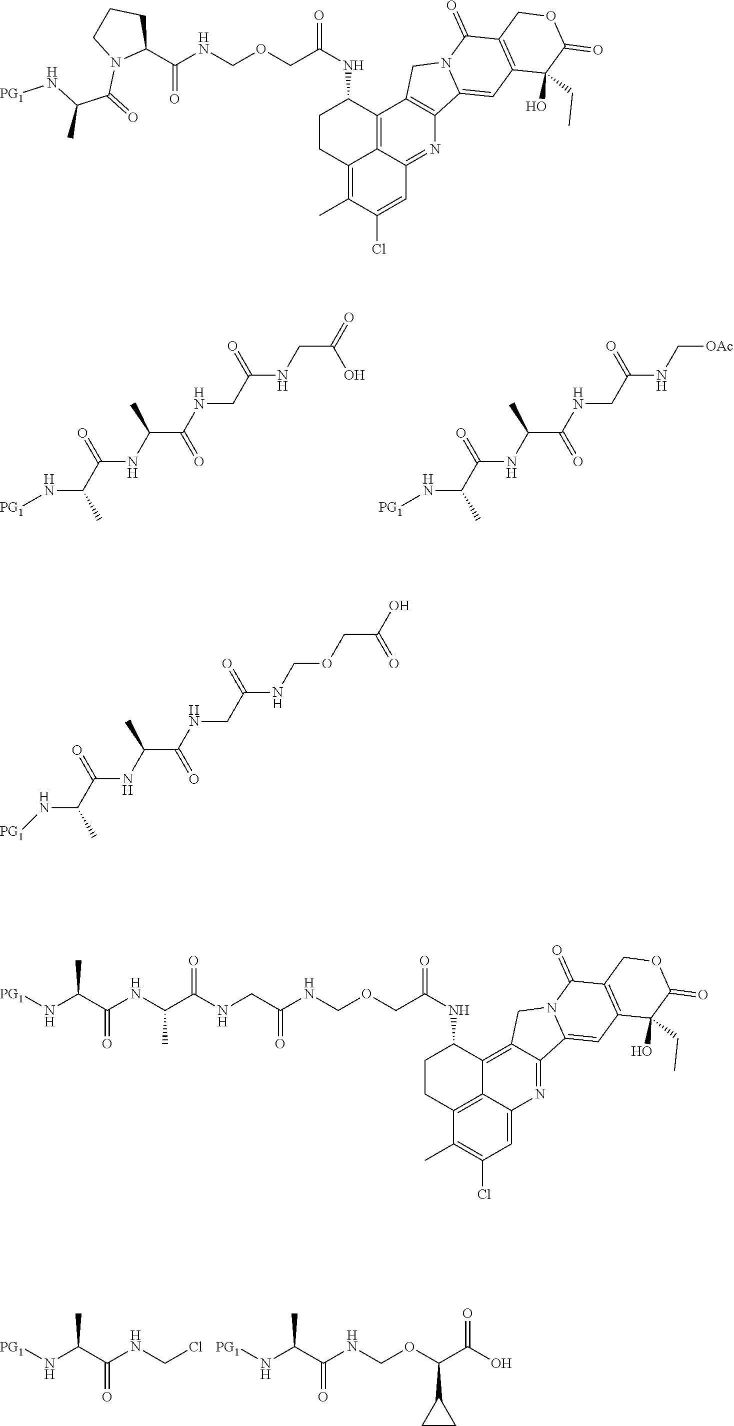

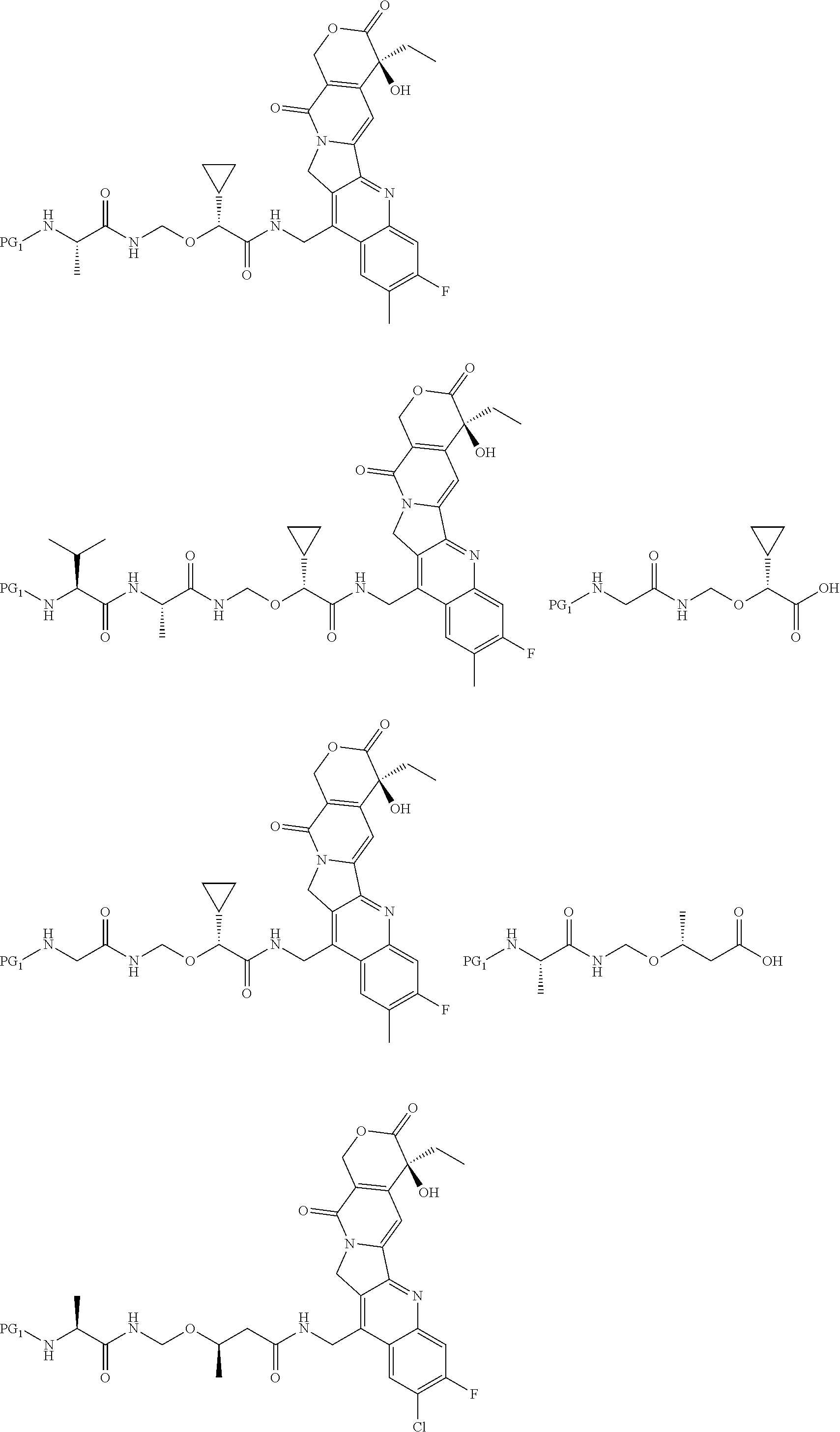

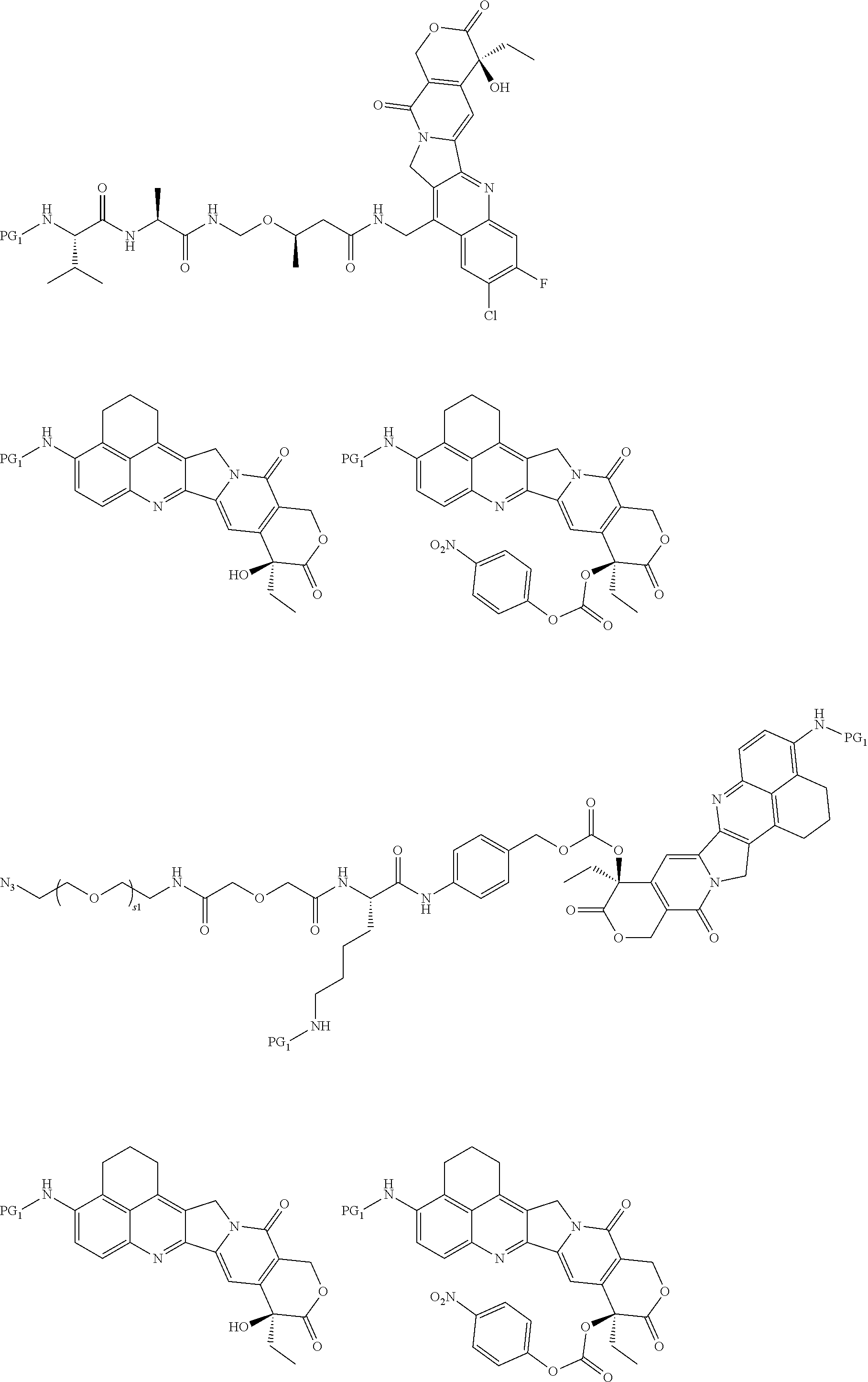

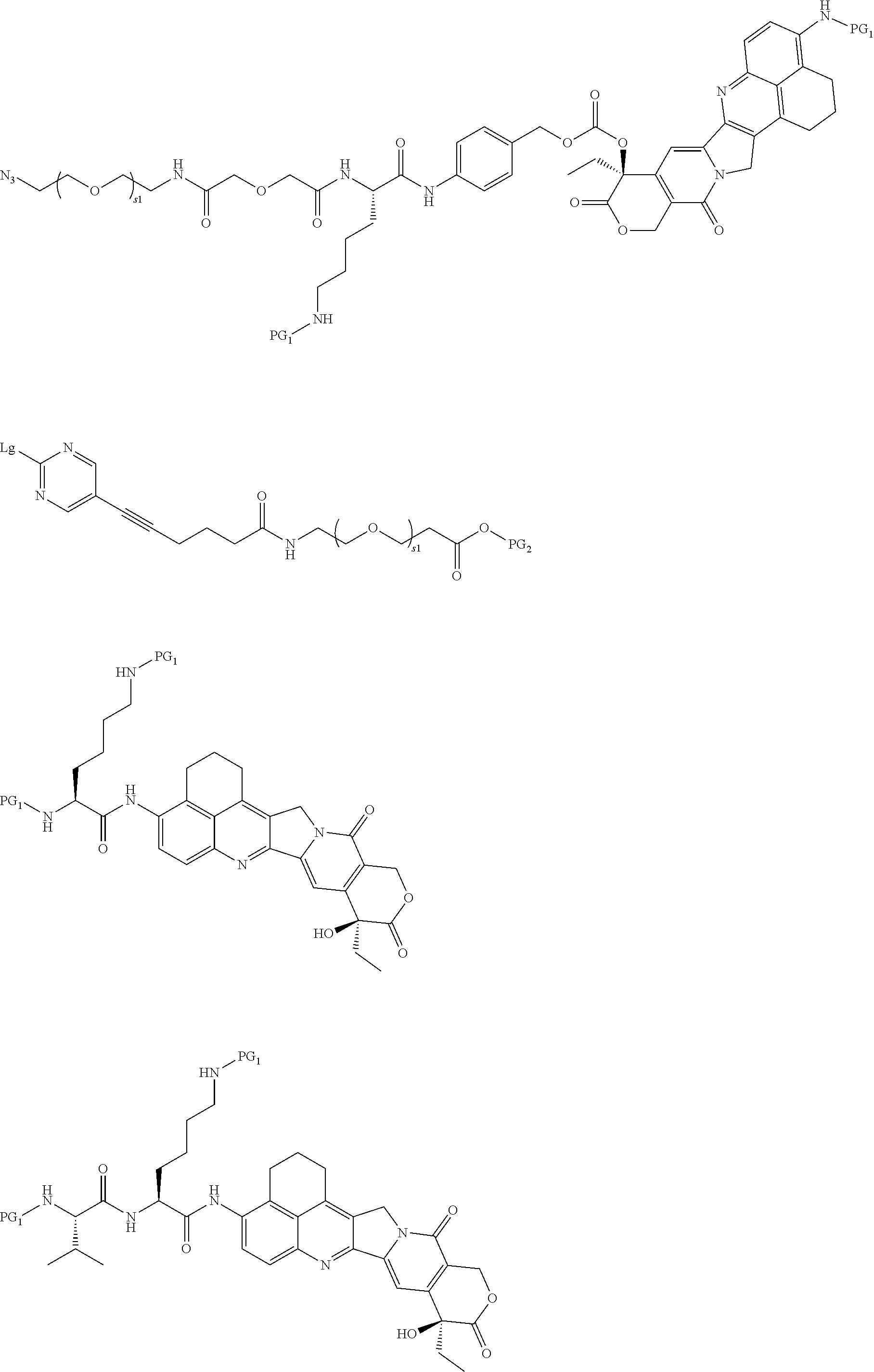

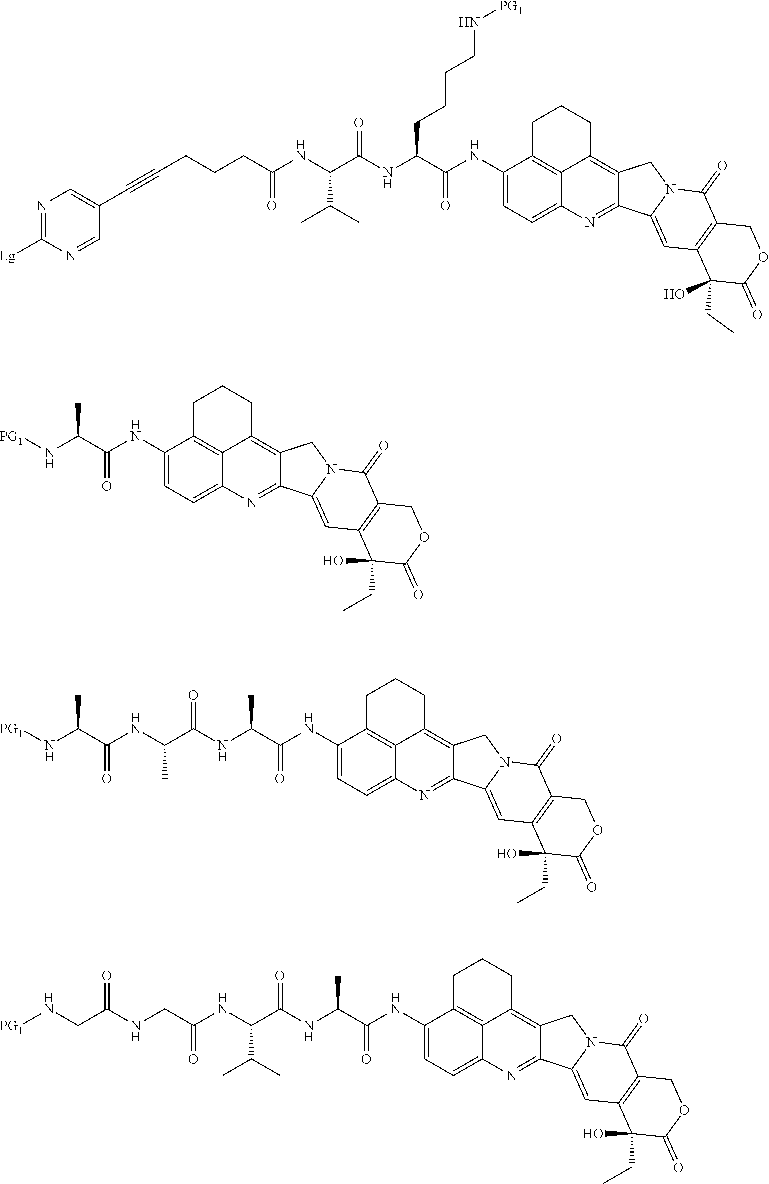

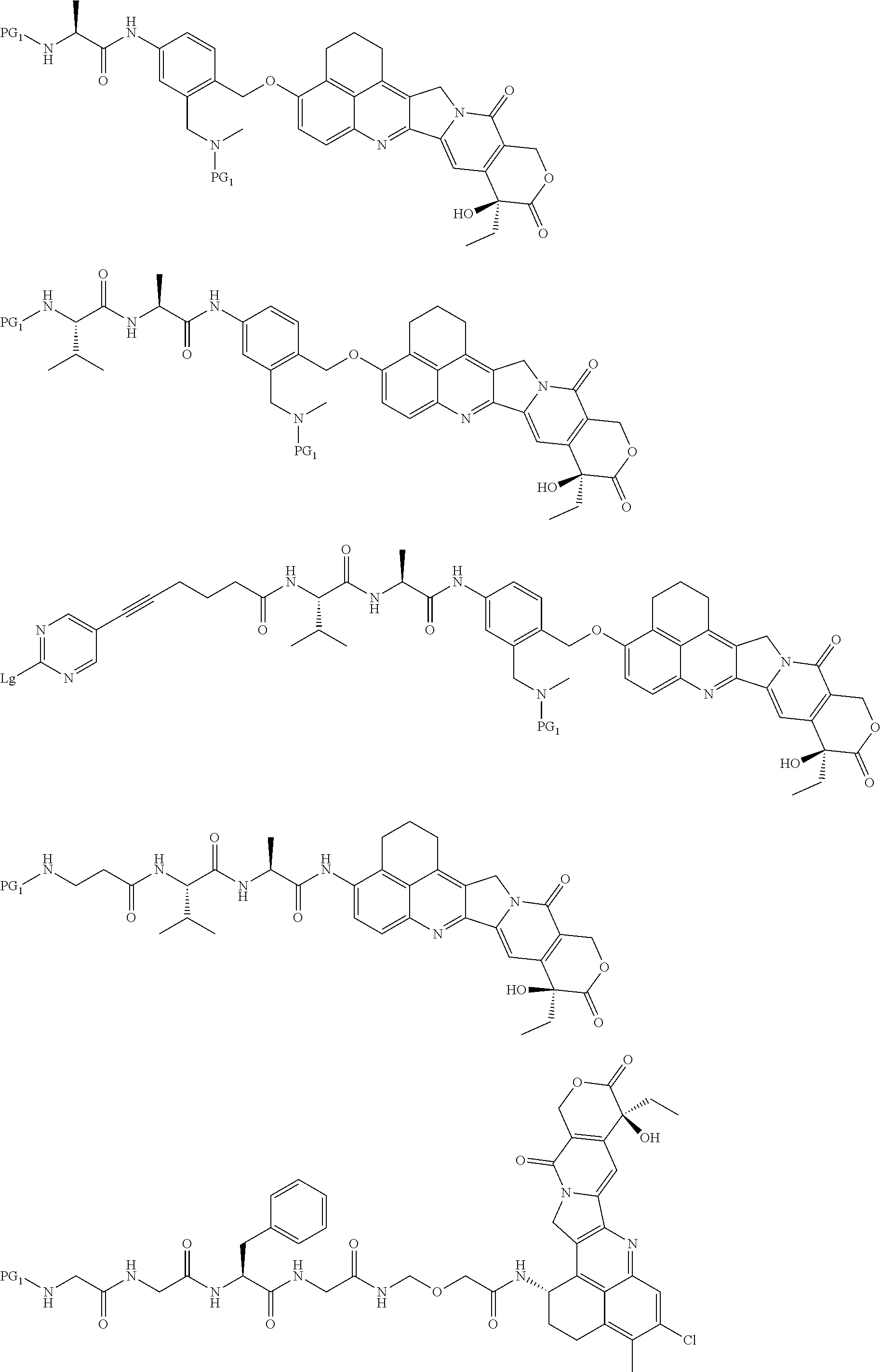

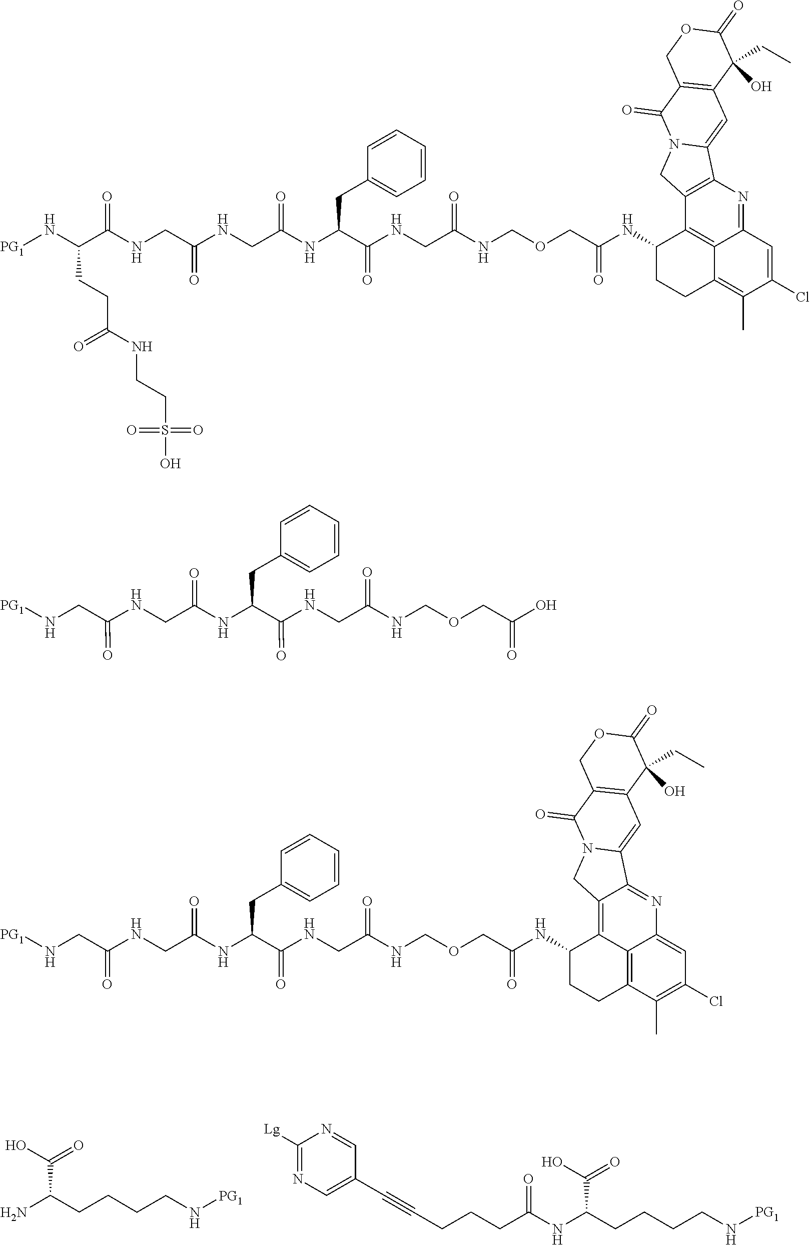

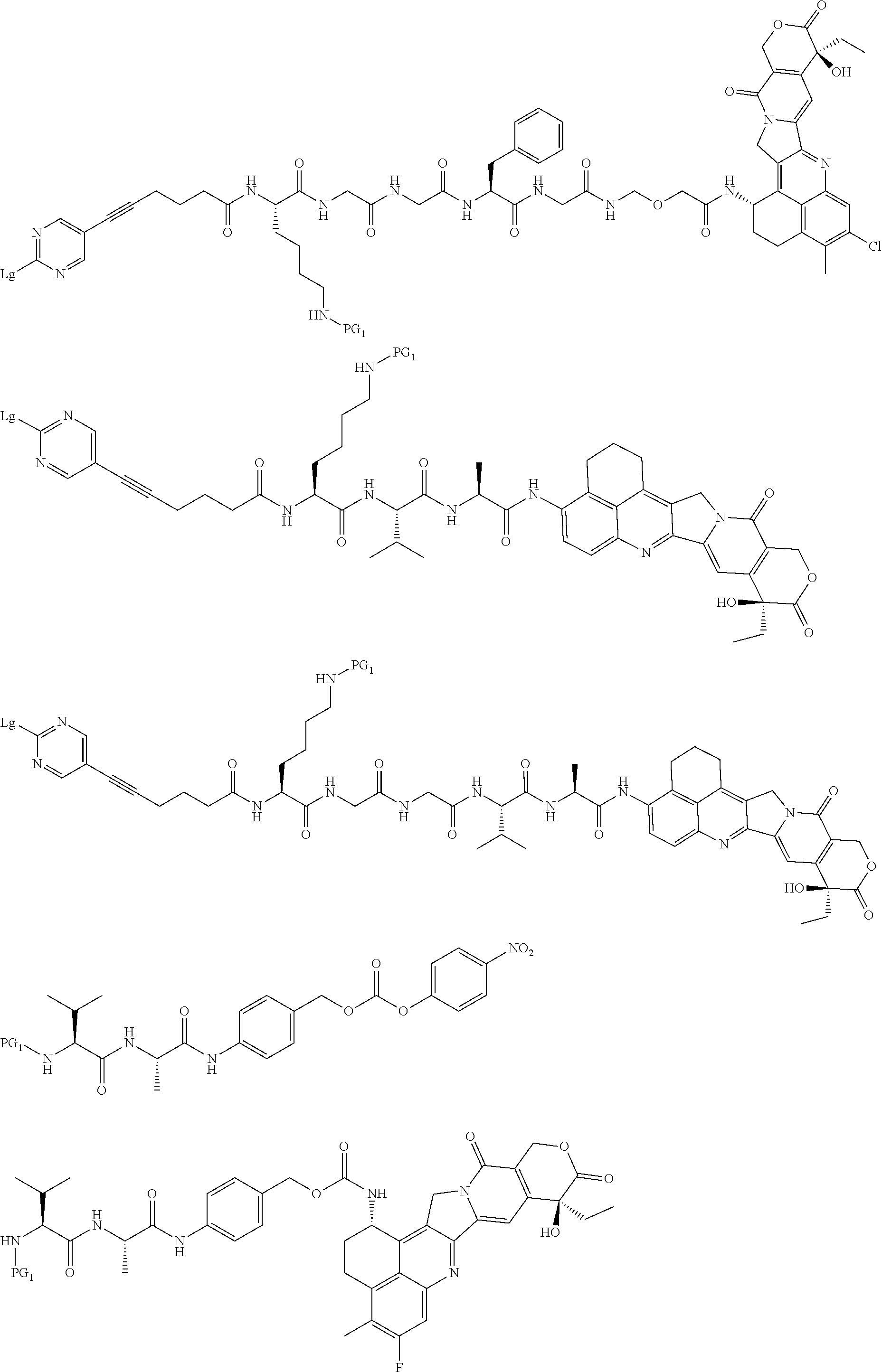

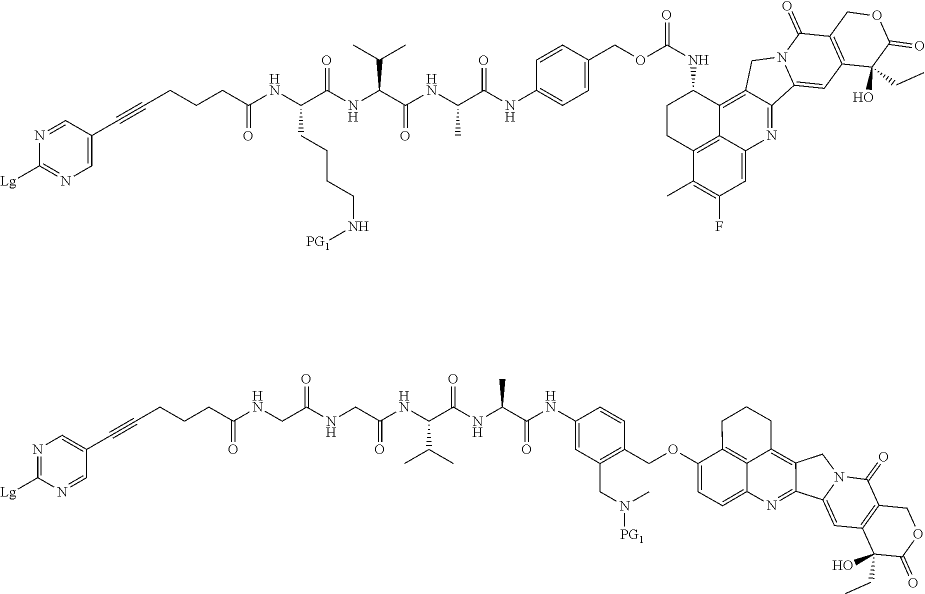

wherein
PG₁ is each independently H or an amino protecting group, the amino protecting group is an alkoxycarbonyl amino protecting group, such as benzyloxycarbonyl (Cbz), tert-butyloxycarbonyl (Boc), fluorenylmethyloxycarbonyl (Fmoc), allyloxycarbonyl (Alloc), trimethylsilylethoxycarbonyl (Teoc), methyl(or ethyl)oxycarbonyl; an acyl amino protecting group, such as phthaloyl (Pht), p-toluenesulfonyl (Tos), trifluoroacetyl (Tfa), o-(p-)nitrobenzenesulfonyl (Ns), pivaloyl, benzoyl, tert-butyloxycarbonyl, 9-fluorenylmethoxycarbonyl, allyloxycarbonyl, trichloroethoxycarbonyl, trimethylsilylethoxycarbonyl, benzyloxycarbonyl, p-toluenesulfonyl, p-nitrobenzenesulfonyl, trifluoroacetyl, methoxycarbonyl, or ethoxycarbonyl; an alkyl amino protecting group, such as trityl (Trt), C₁₋₆ alkyl-substituted trityl, p-methoxytrityl (MMT), dimethoxytrityl (DMT), 2,4-dimethoxybenzyl (Dmb), 4-methoxybenzyl (PMB), benzyl (Bn);
PG₂ is each independently H or a carboxyl protecting group, such as C₁₋₆ alkyl, allyl, benzyl, 2,4-dimethoxybenzyl, p-methoxybenzyl, methoxyethoxymethyl, pentafluorophenyl, 4-p-methylbenzyloxybenzyl;
PG₃ is each independently H or a hydroxyl protecting group, such as trimethylsilyl (TMS), triethylsilyl (TES), triisopropylsilyl (TIPS), tert-butyldimethylsilyl (TBS), tert-butyldiphenylsilyl (TBDPS), methyl, tert-butyl, allyl, benzyl, trityl (Trt), methoxymethyl (MOM), ethoxyethyl, 2-tetrahydropyranyl (THP), formyl, acetyl, benzoyl, or p-nitrobenzoyl;
s1 is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20.
Lg is as defined above.

In some embodiments, the present application provides intermediate compounds having the structures shown below, or salts, stereoisomers, tautomers or isotopically labeled compounds thereof:

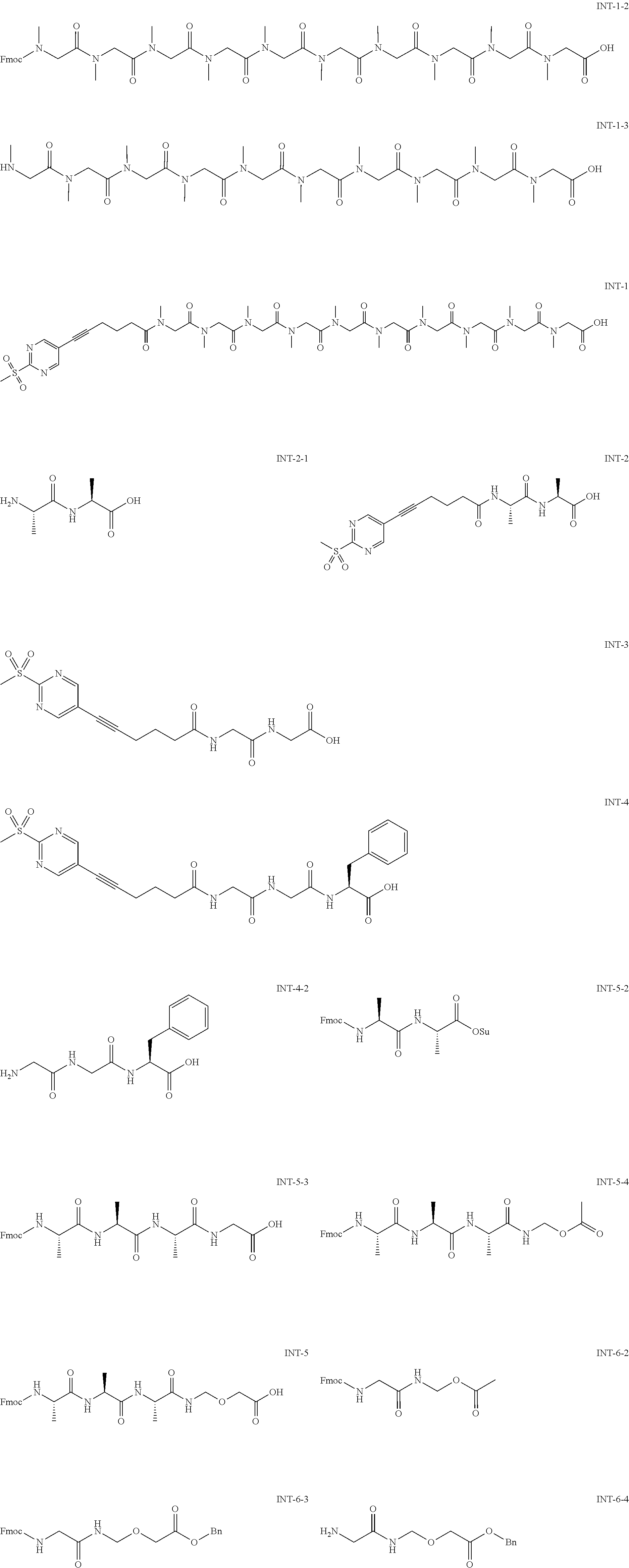

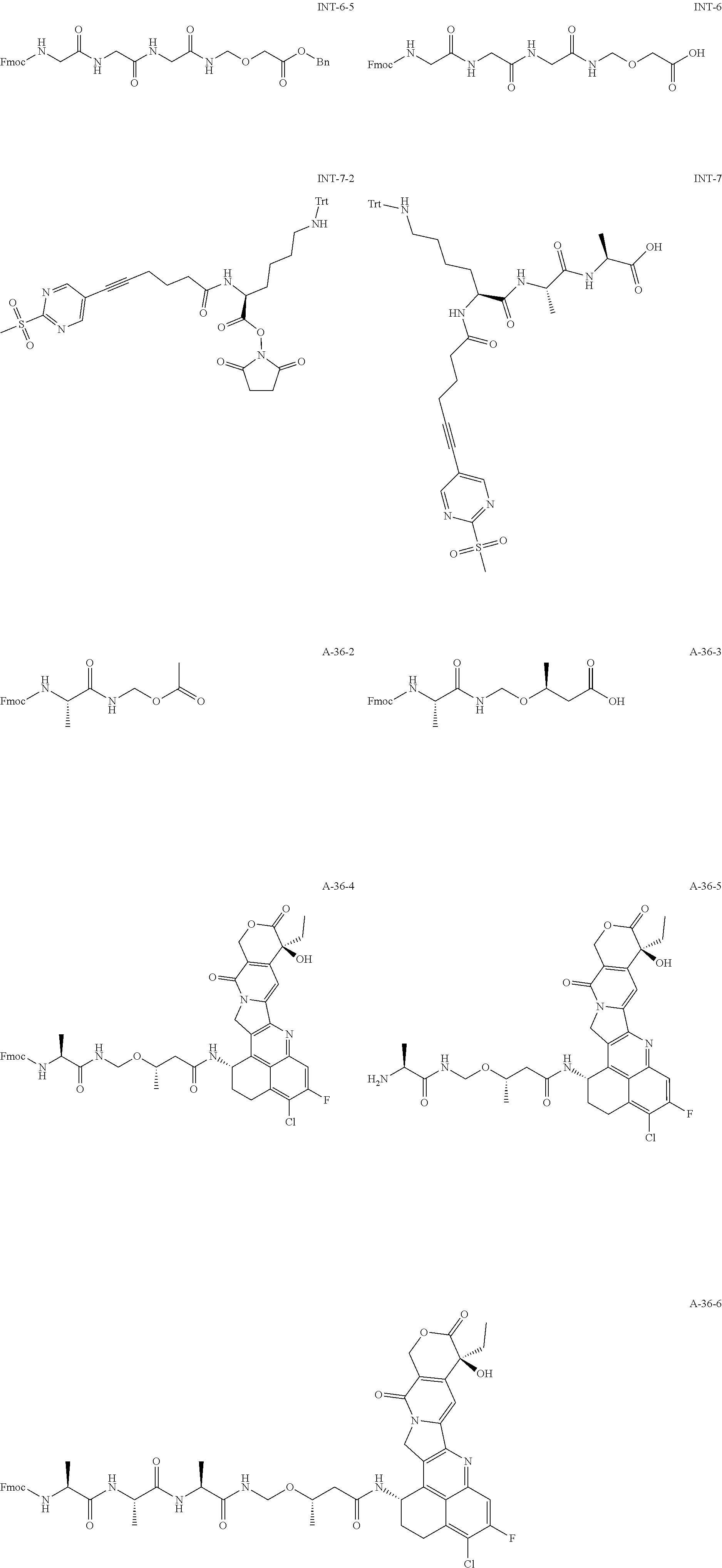

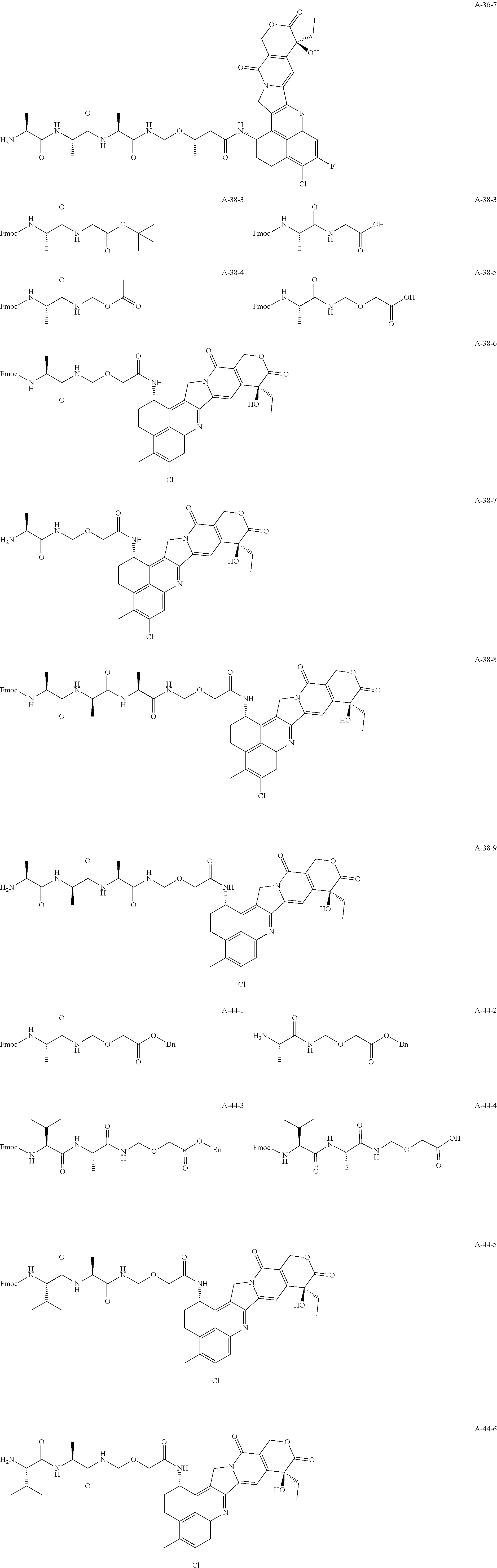

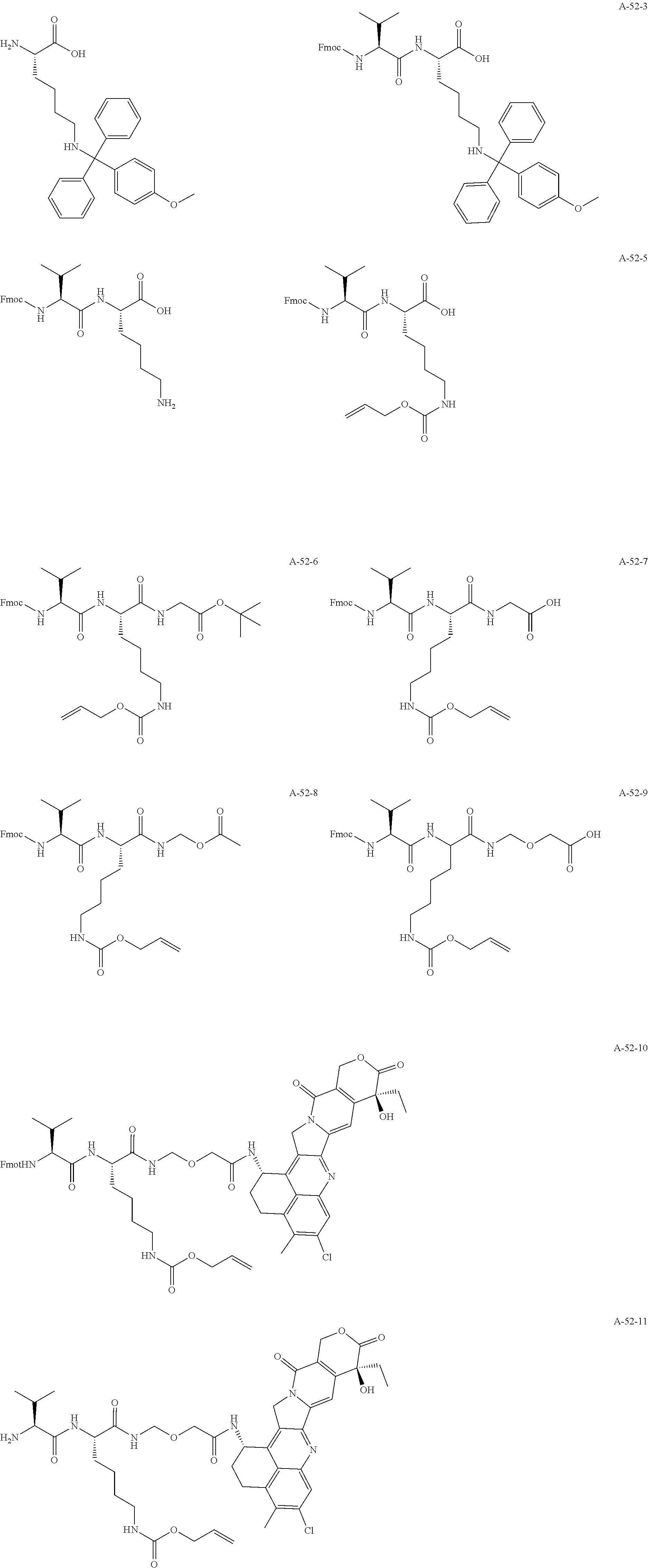

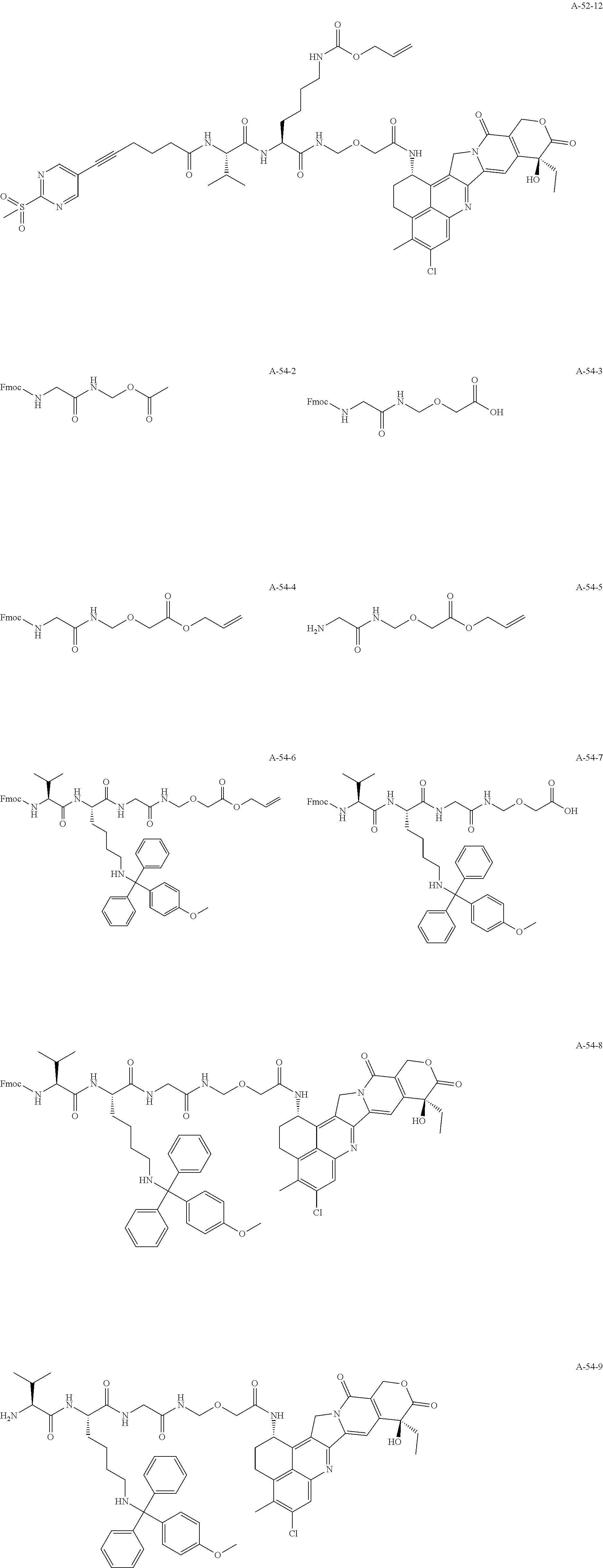

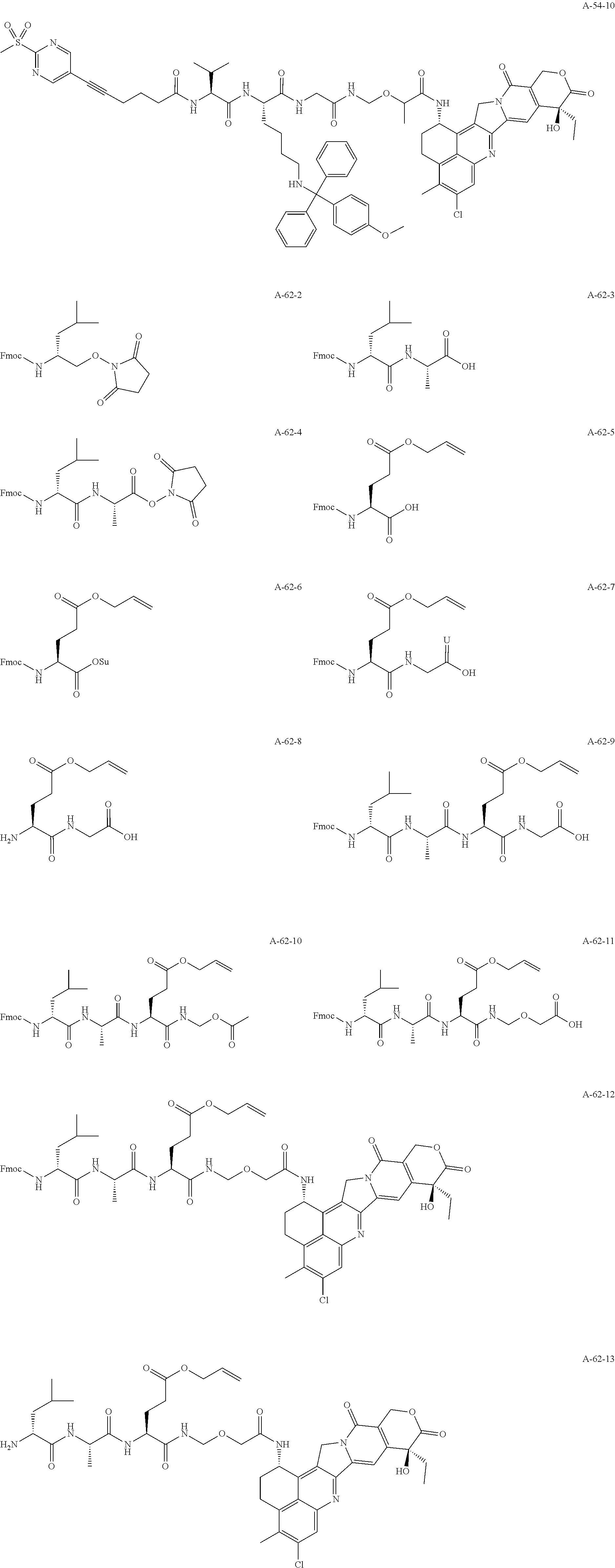

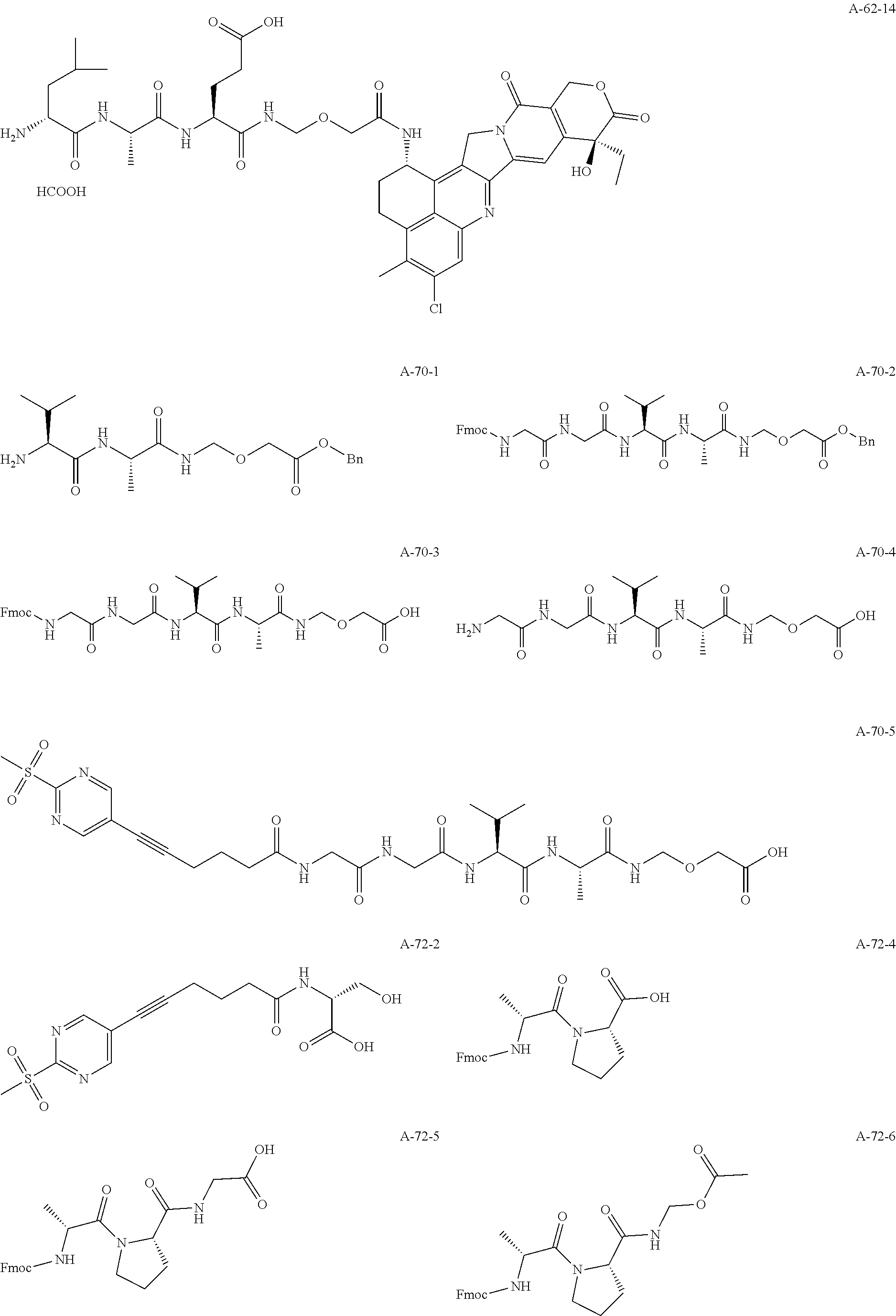

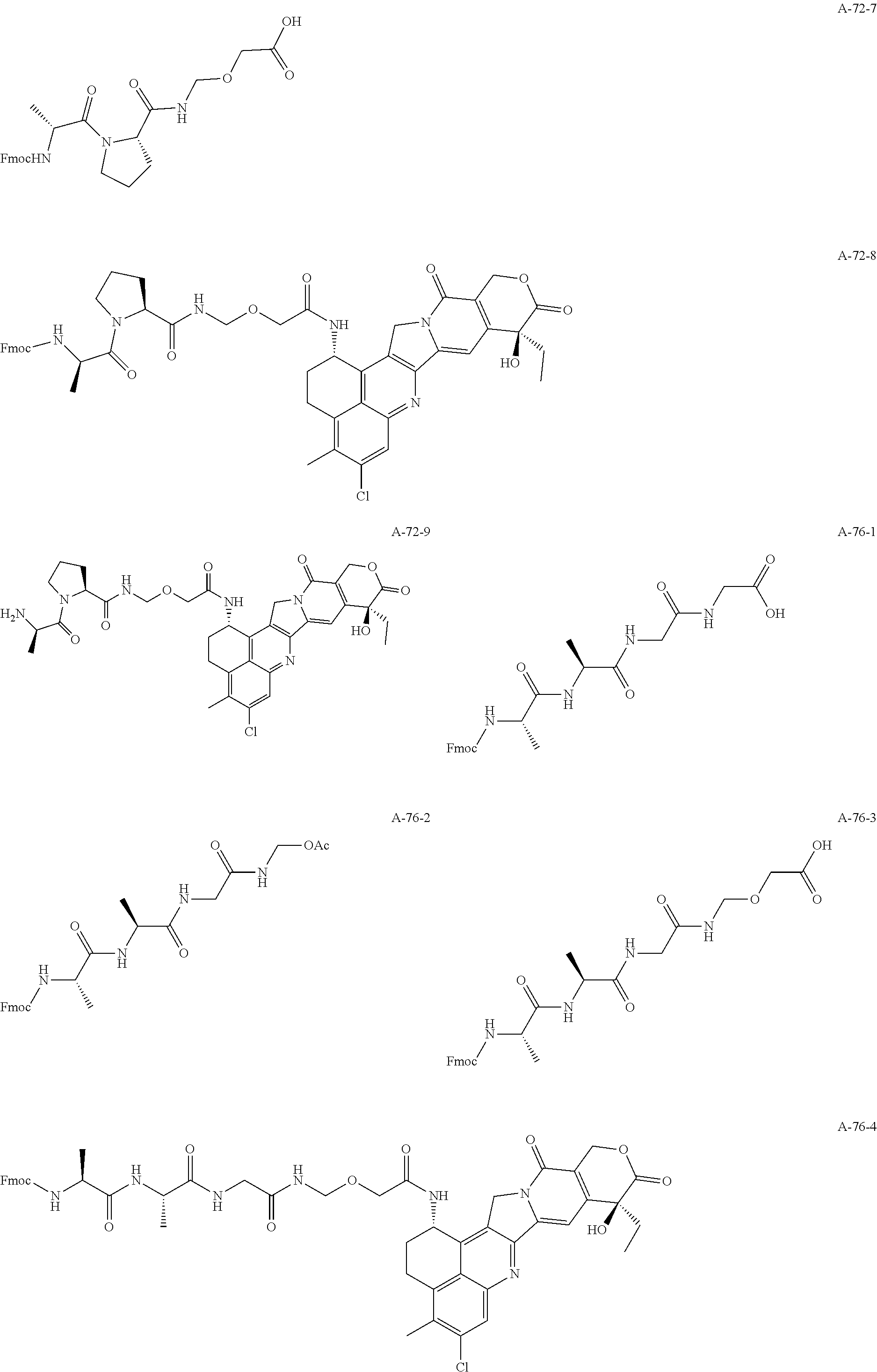

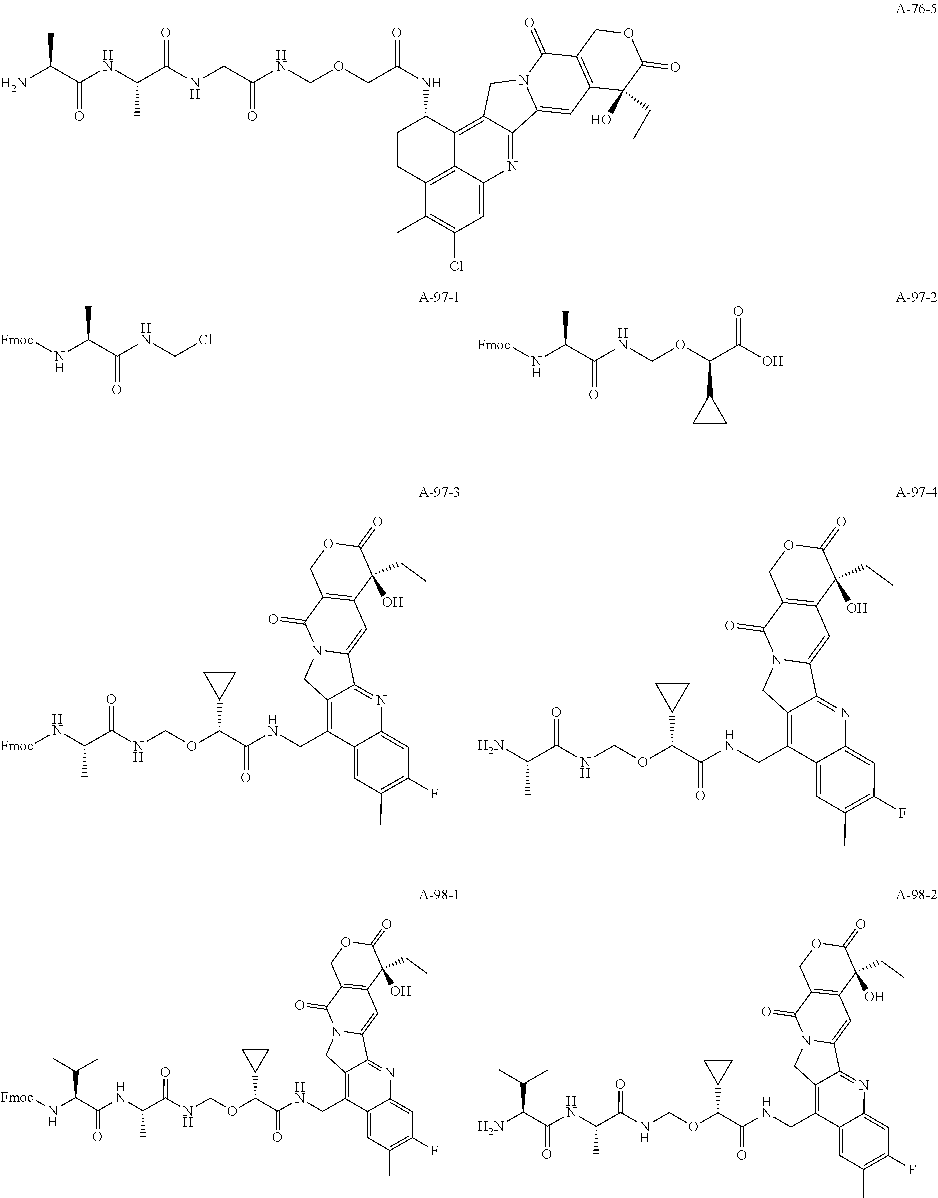

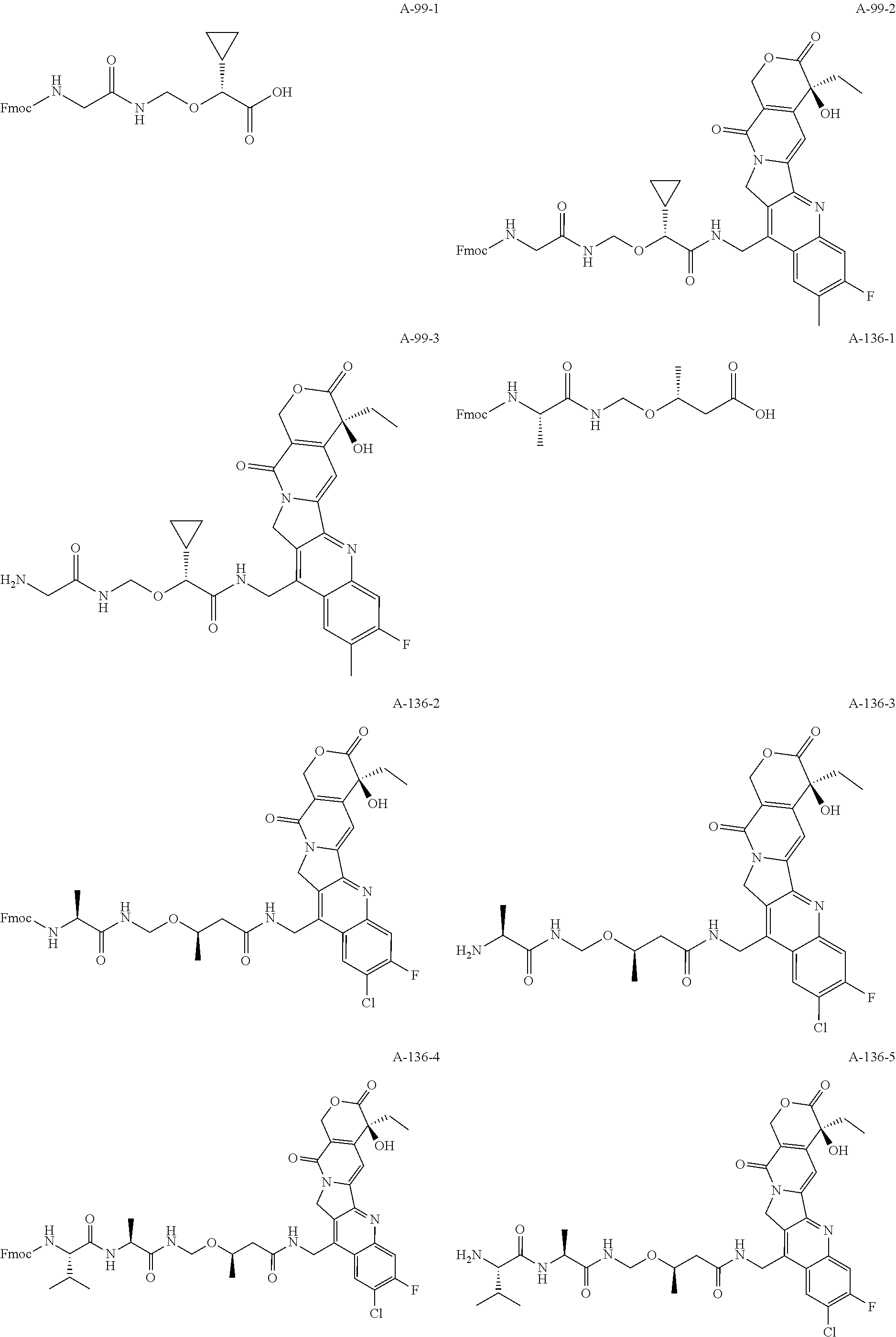

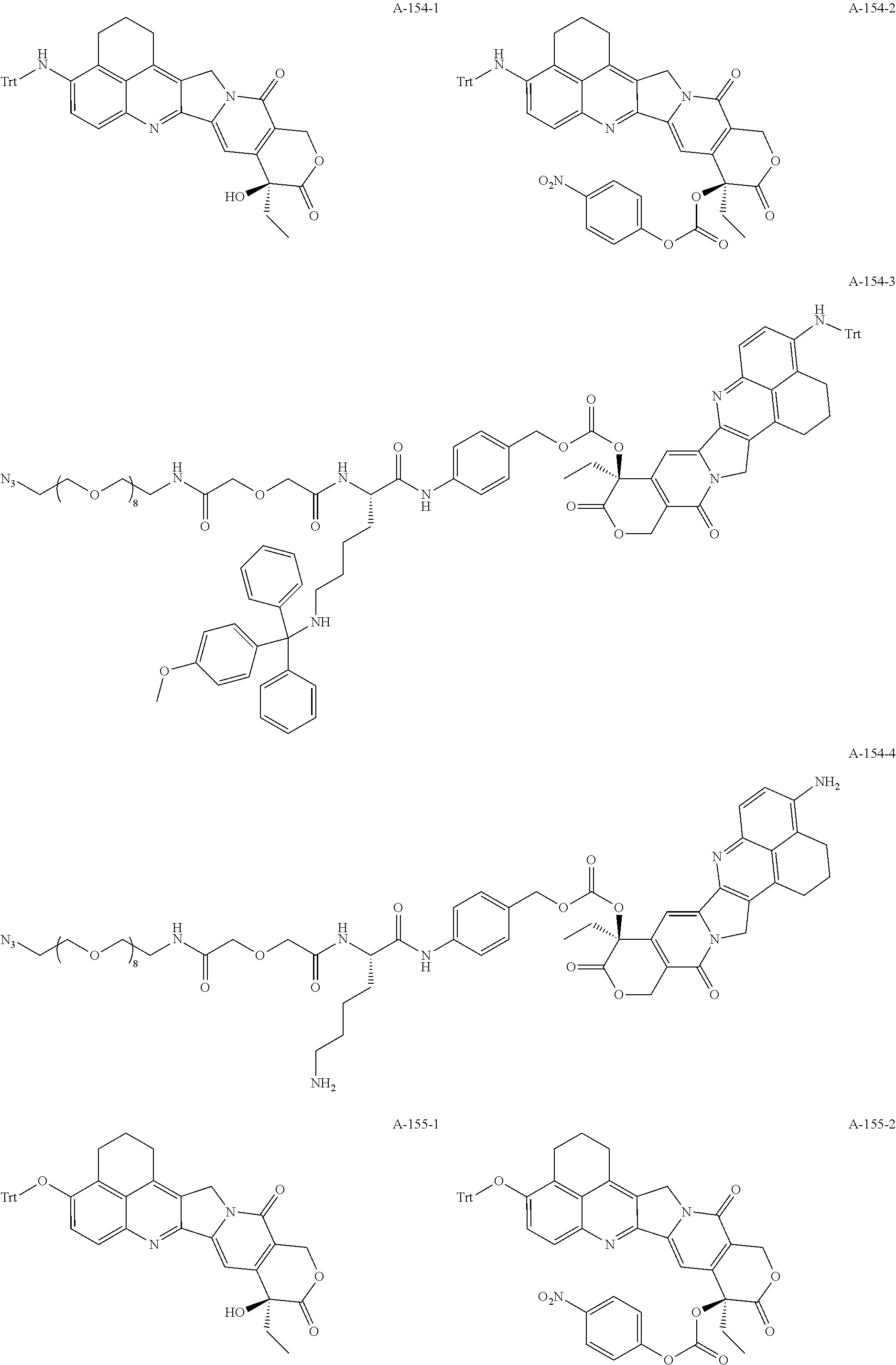

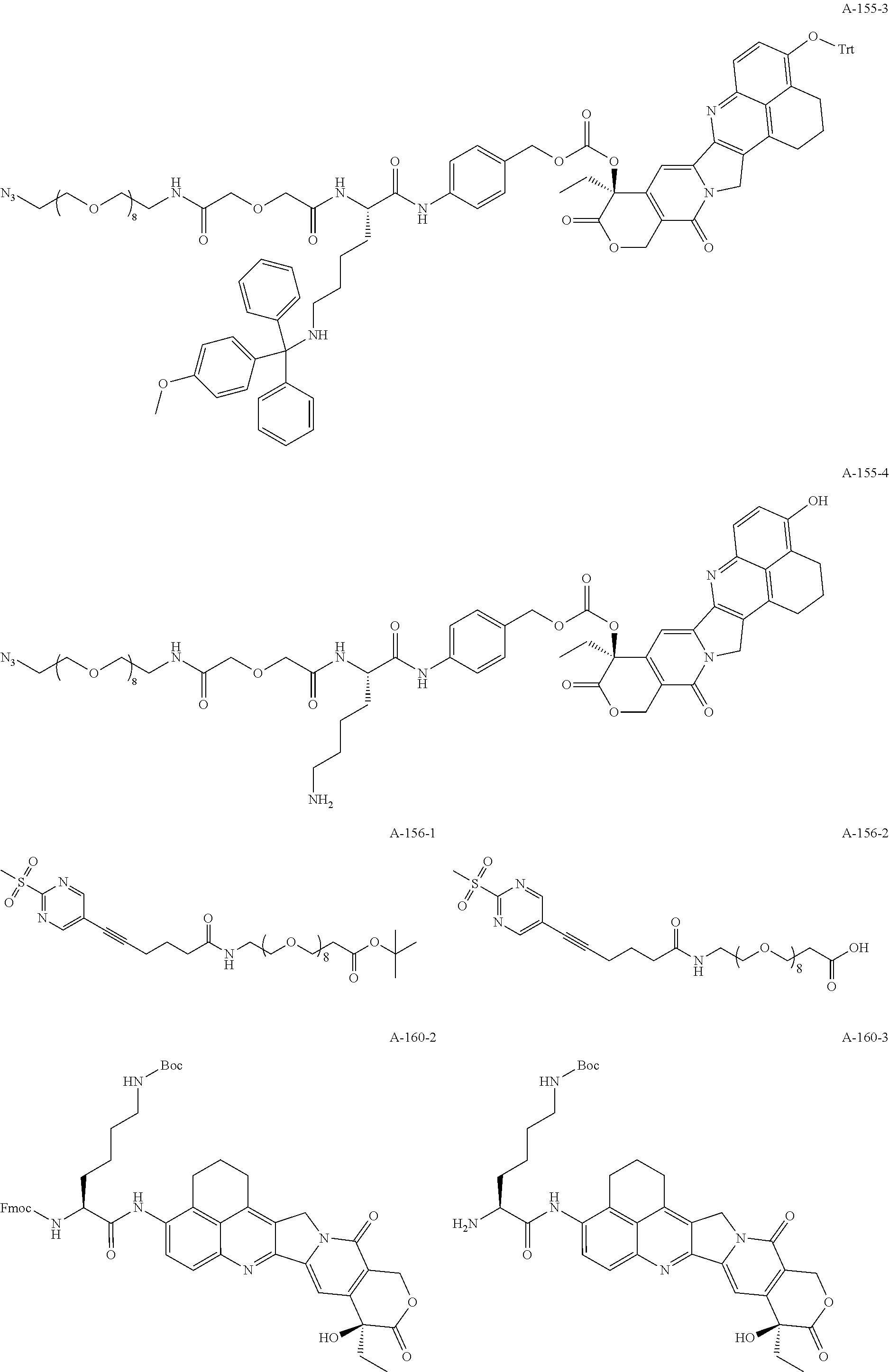

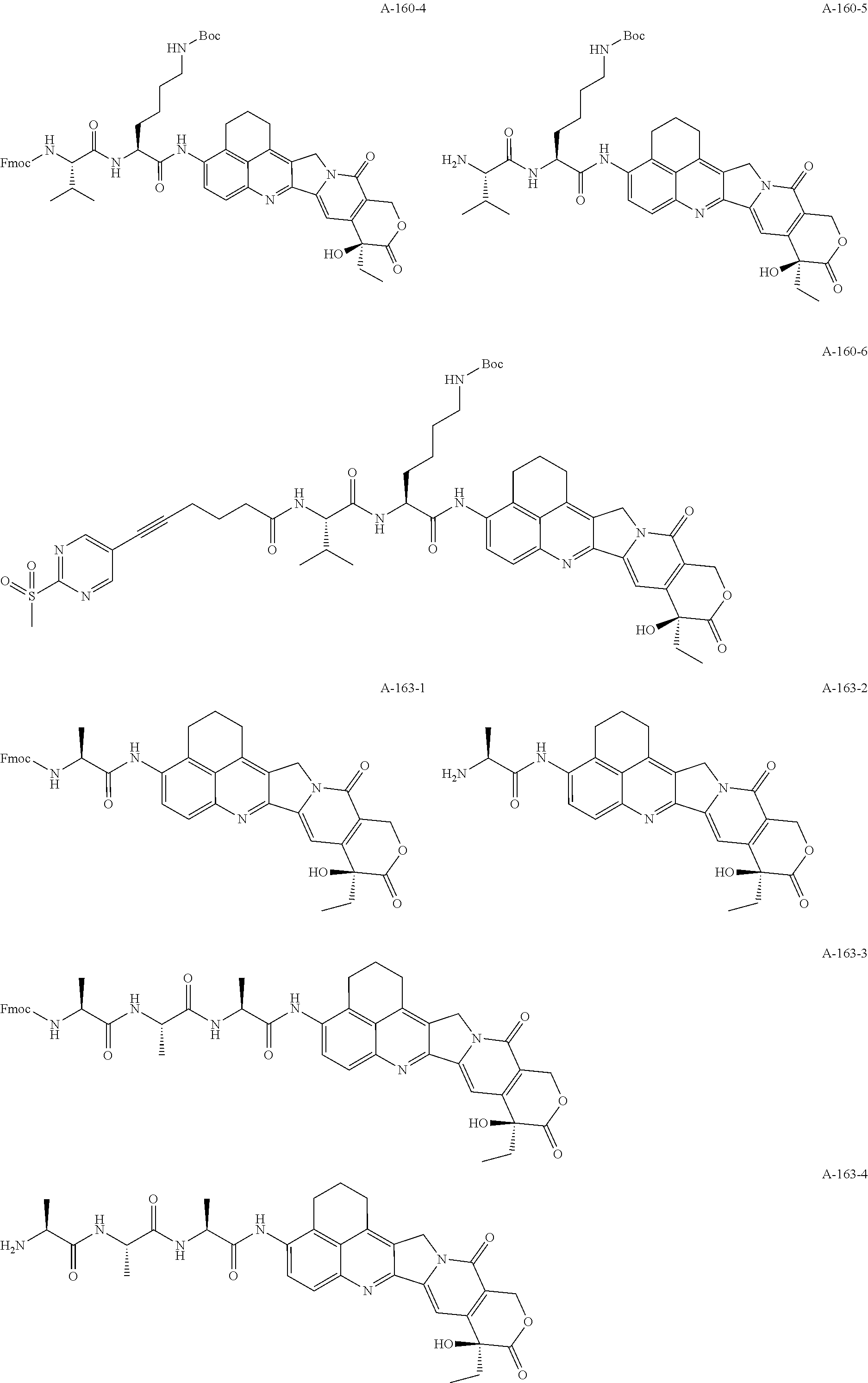

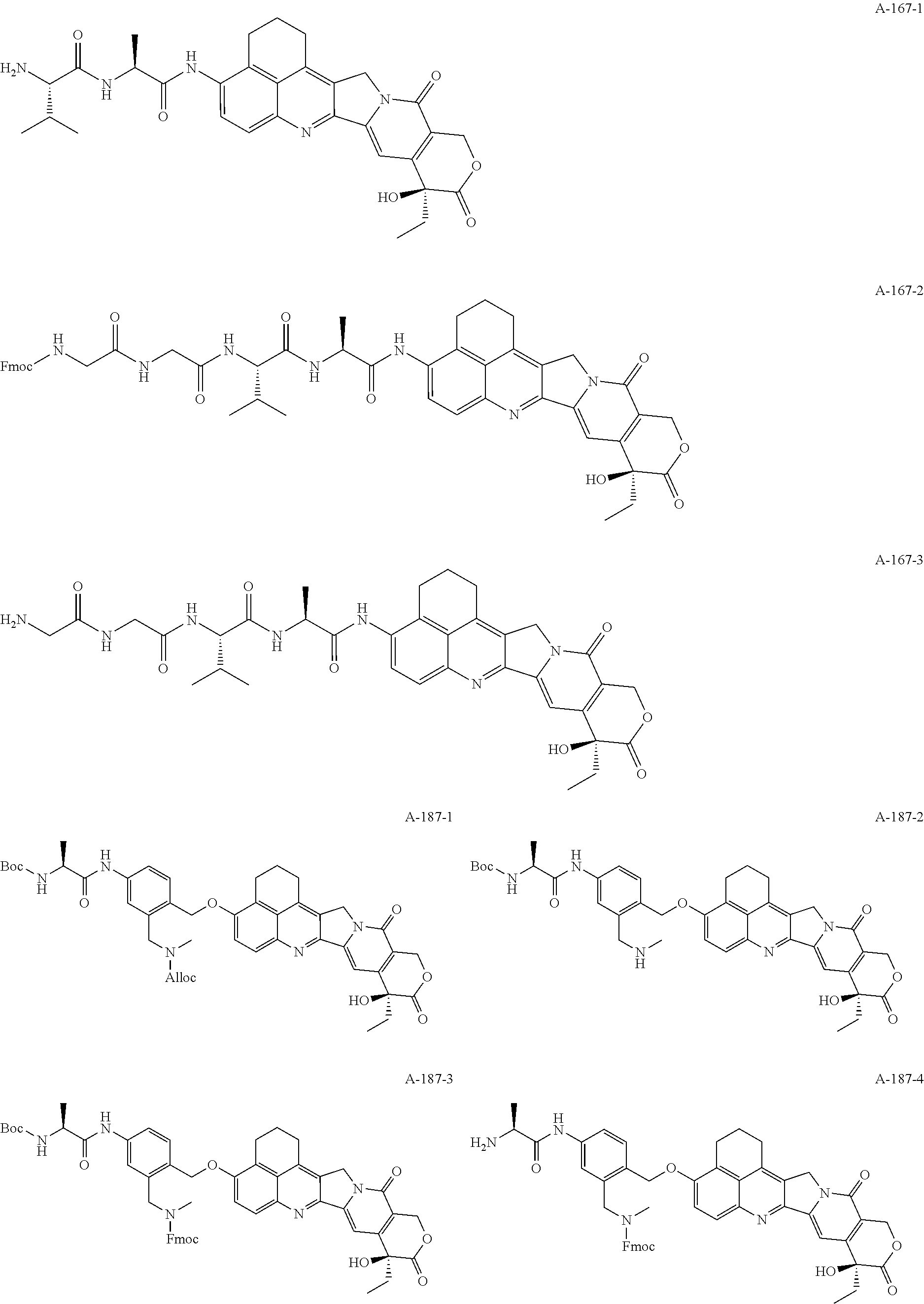

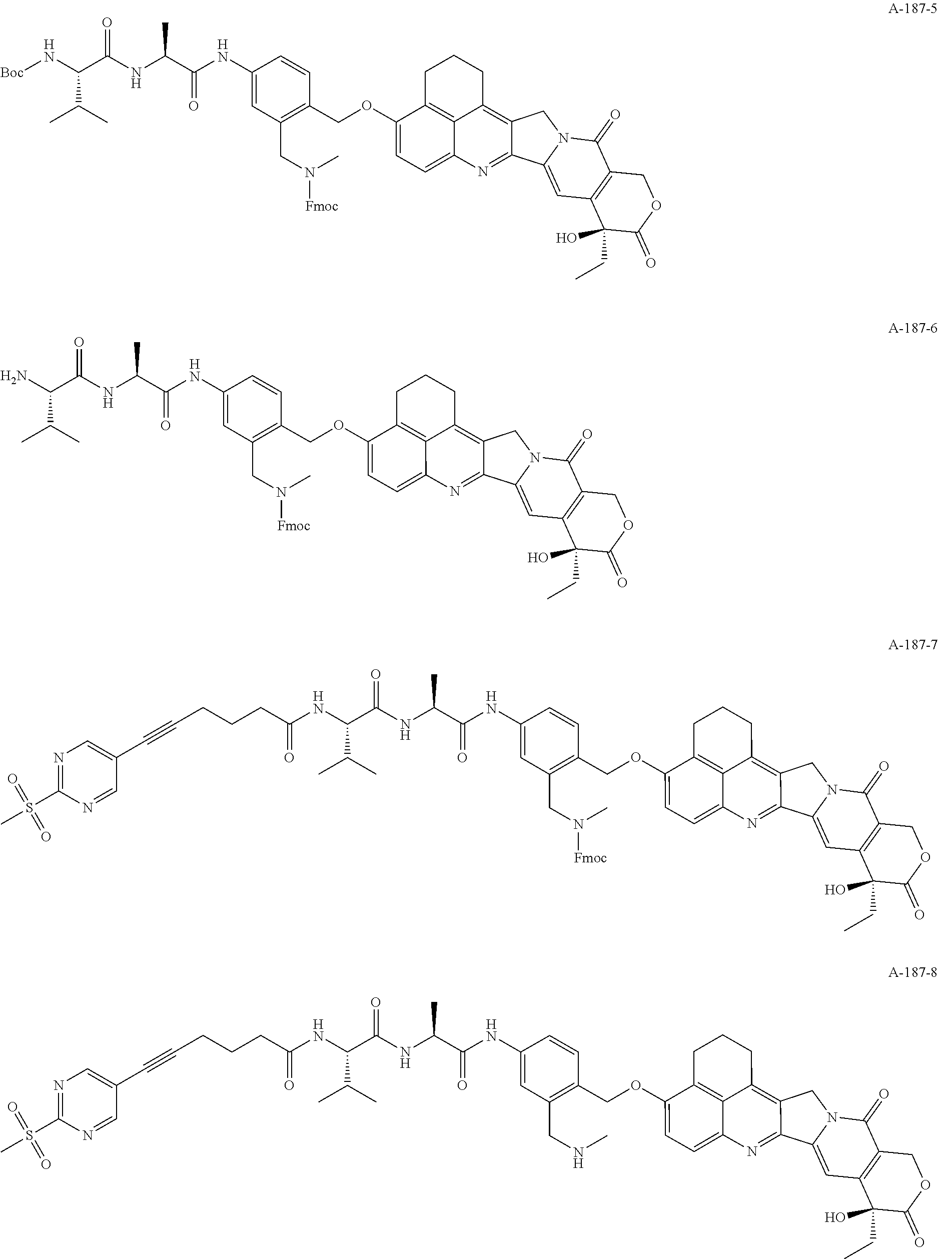

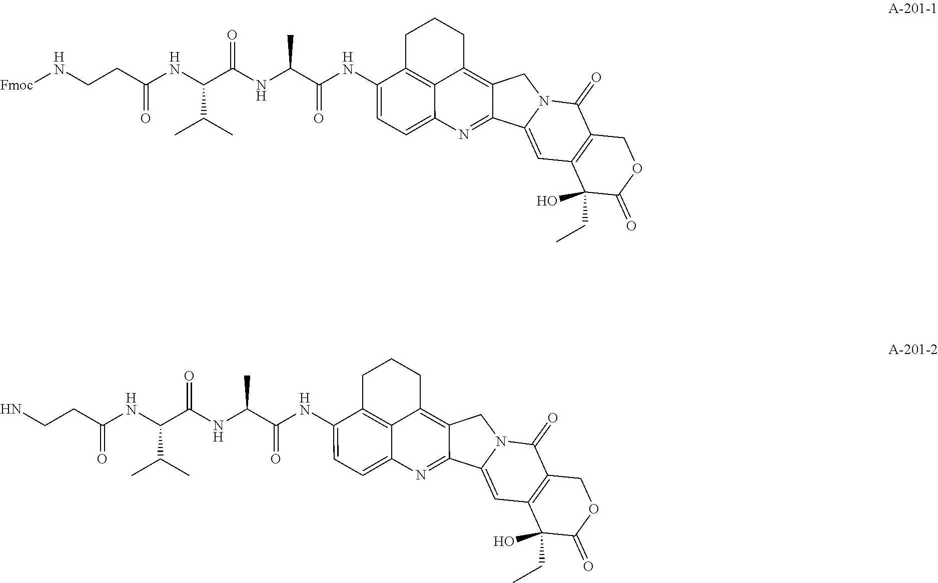

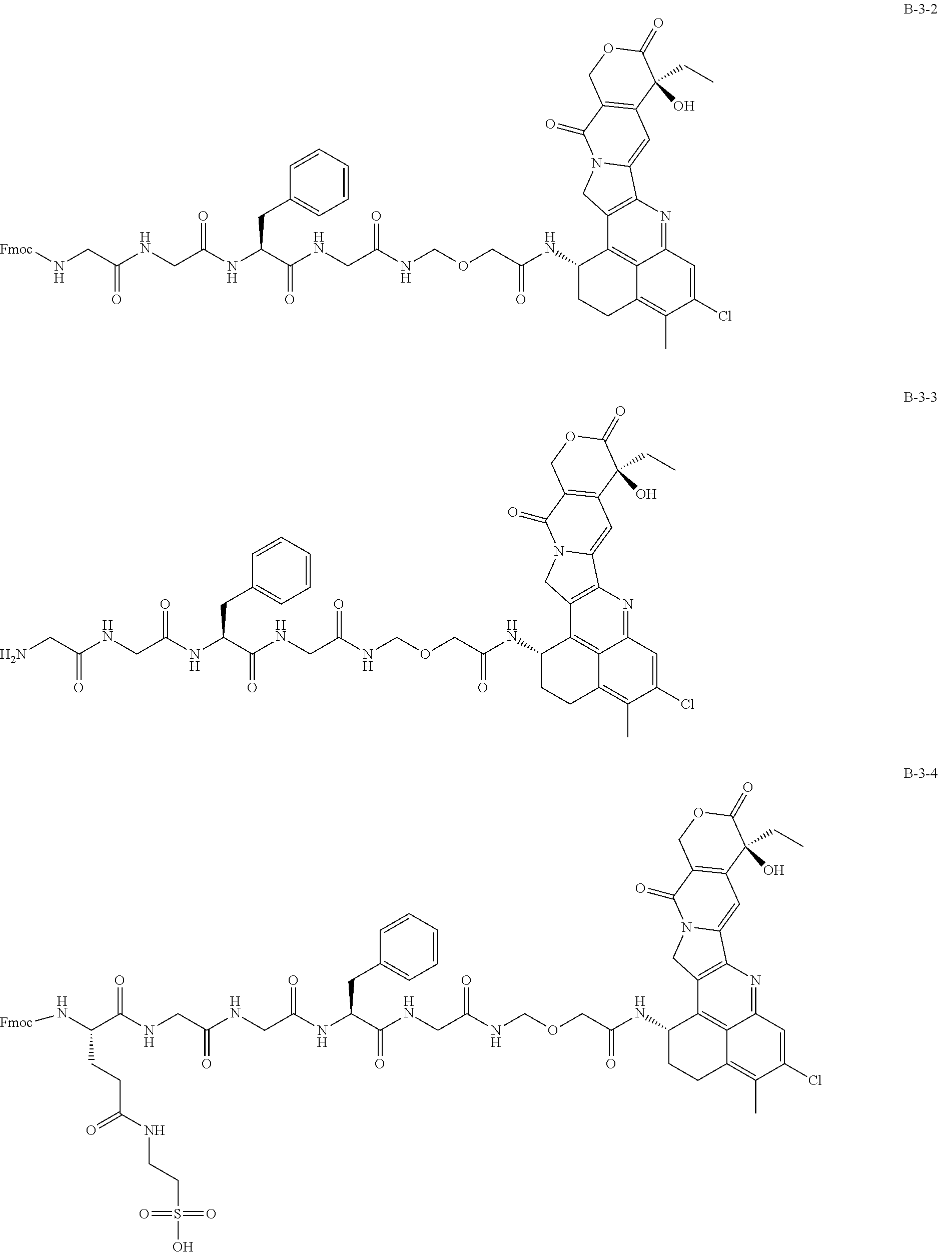

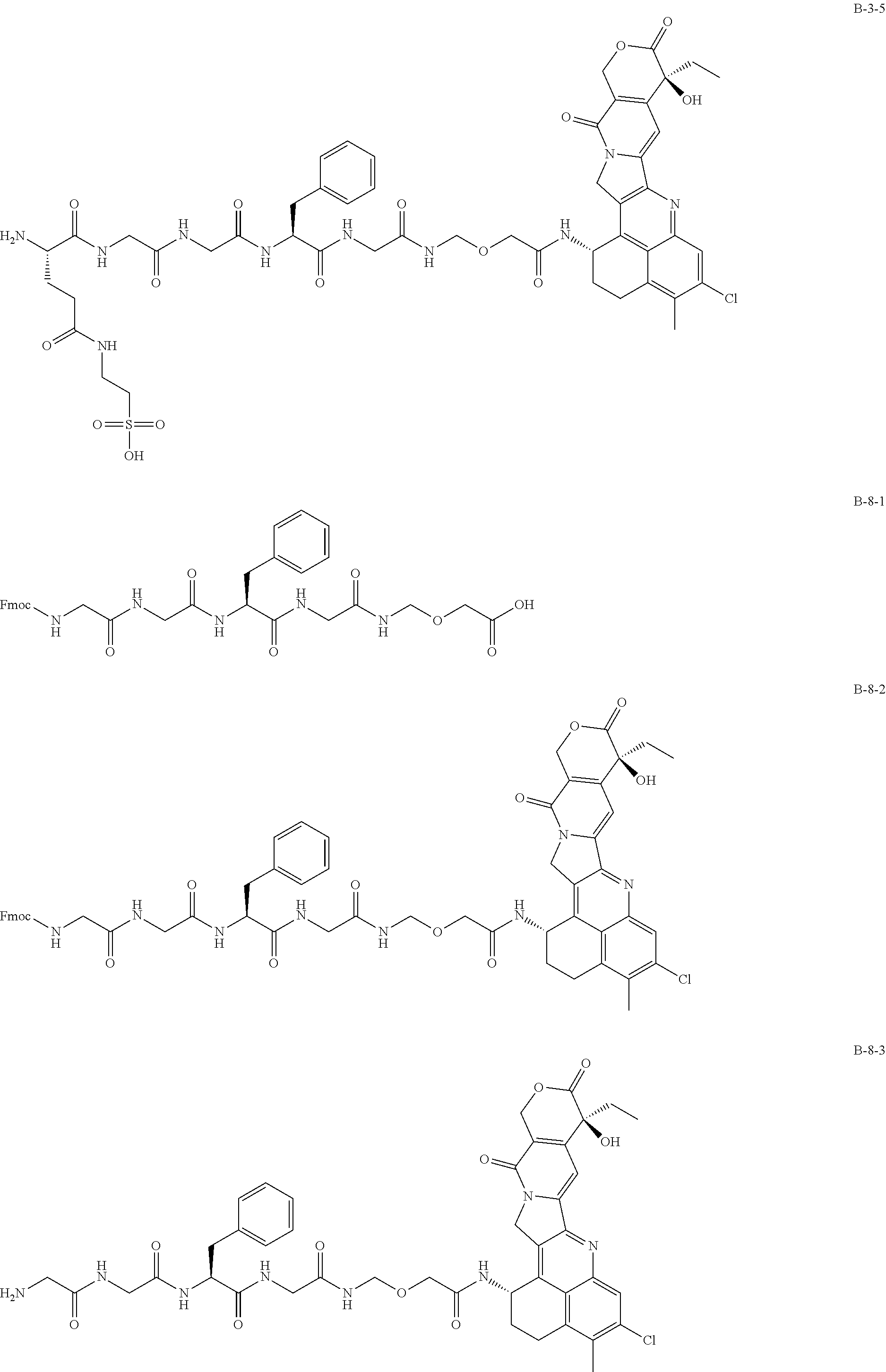

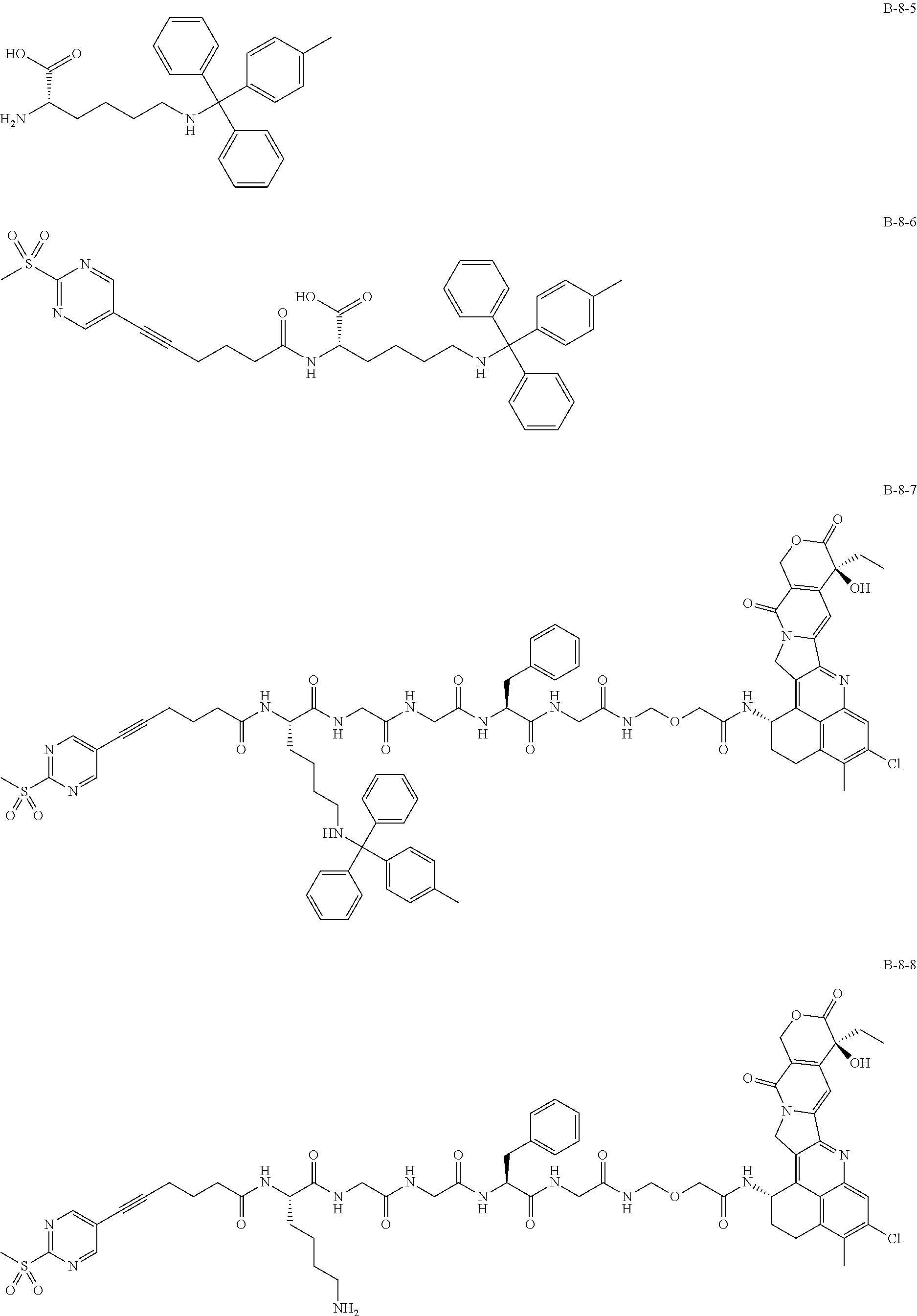

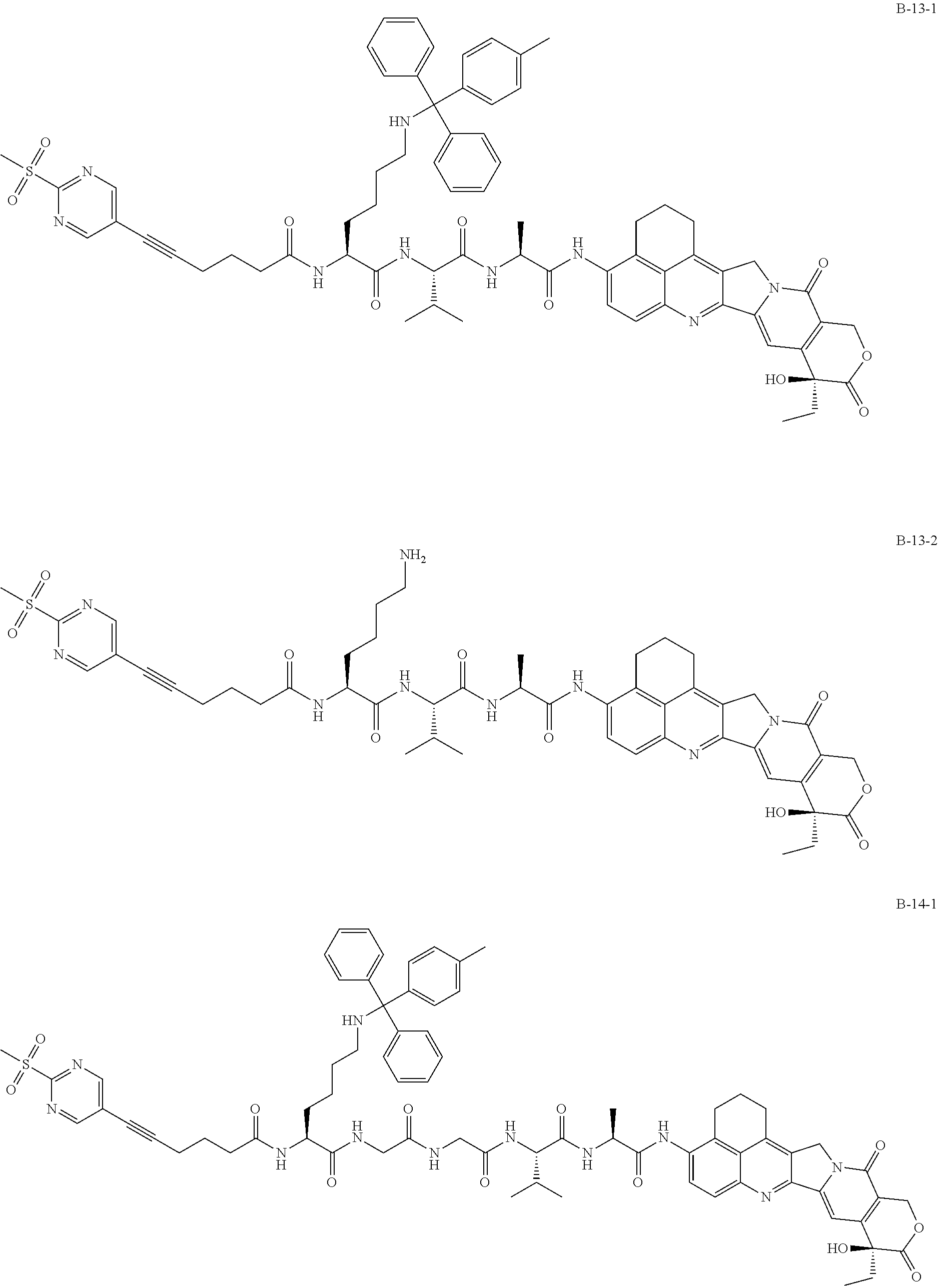

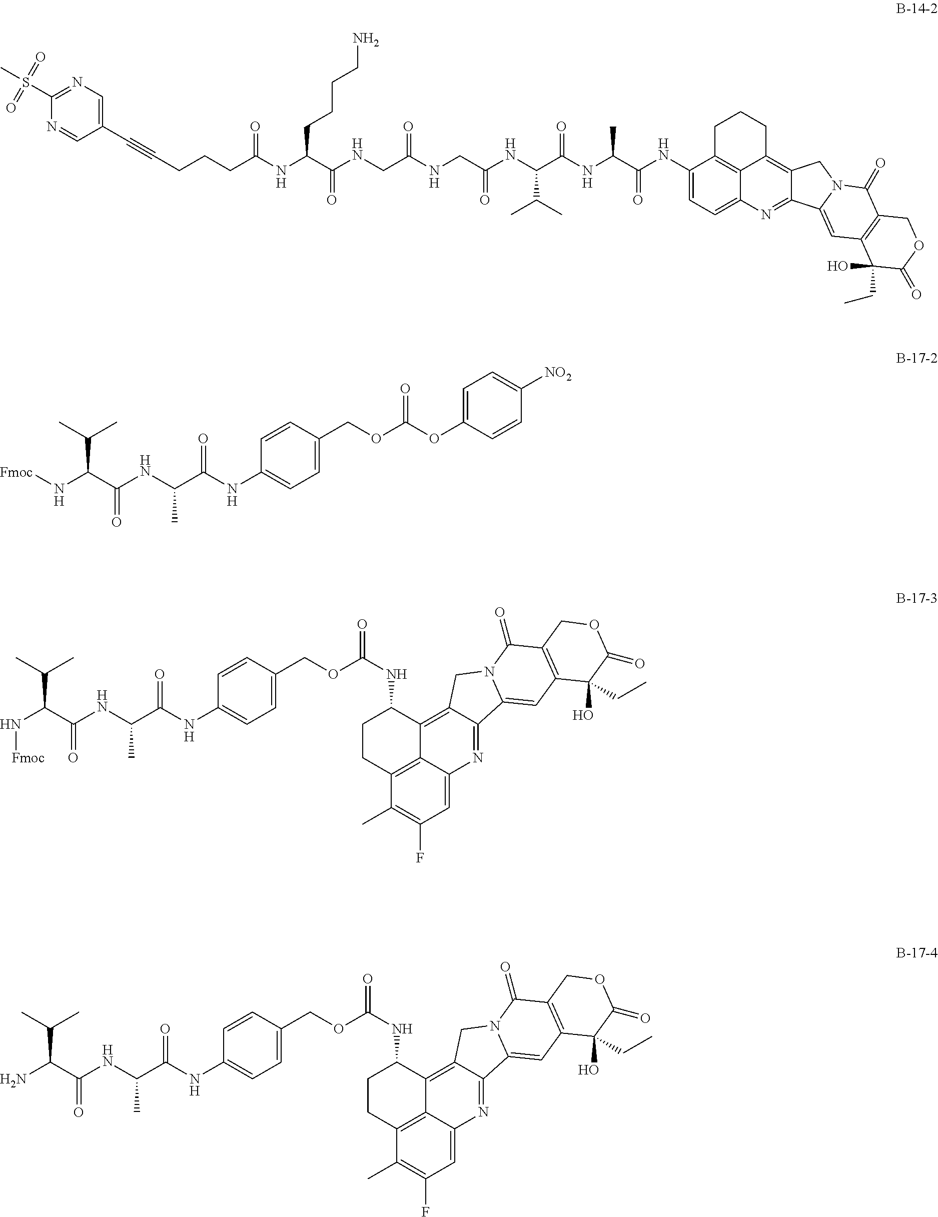

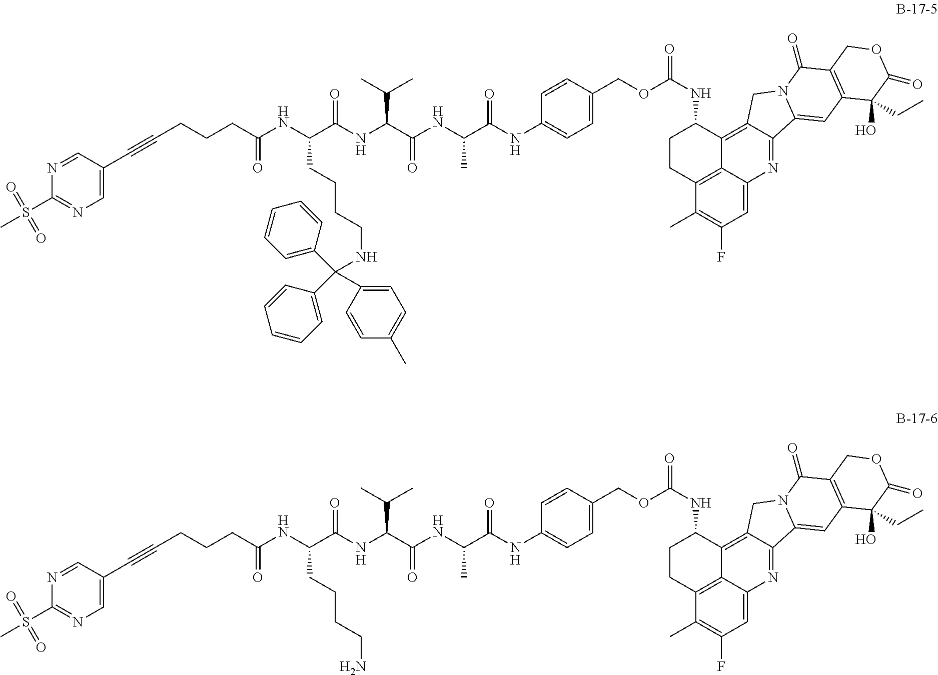

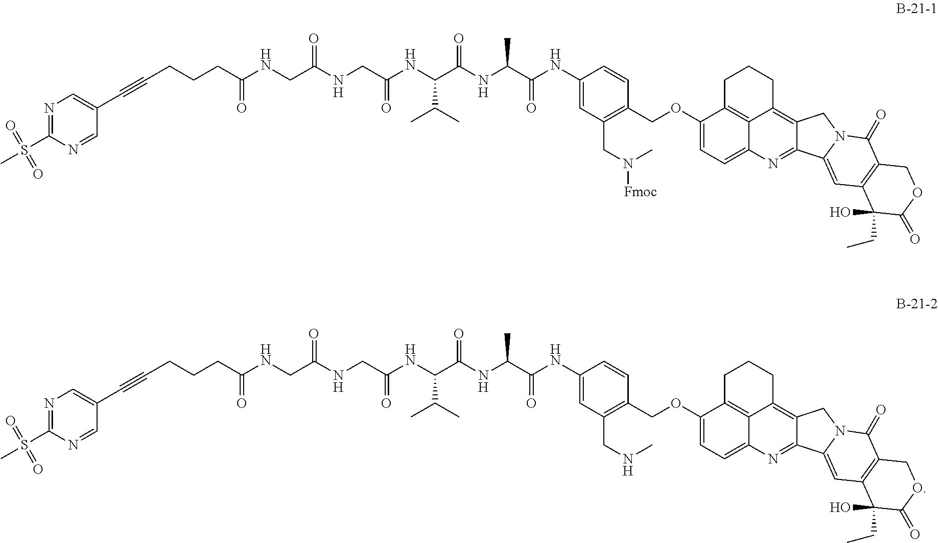

In another aspect, the present application provides a use of the intermediate compounds or salts, stereoisomers, tautomers or isotopically labeled compounds thereof as described above in the manufacture of the compound or pharmaceutically acceptable salt thereof the present invention.

### Composition

On the other hand, the present application provides a composition, which may comprise a plurality of ADCs described herein. Each antibody molecule in the composition can be conjugated with 1, 2, 3, 4, 5, 6, 7, 8, 9 or 10 compounds as described in the present invention. Therefore, the composition is characterized in that the “drug-to-antibody ratio” (DAR) is in the range of about 1 to about 10. Methods for determining DAR are well known to the technicians in the art, including methods using reverse phase chromatography or HPLC-MS.

For example, in any embodiment, the composition described herein has a DAR of about 1 to about 10, or any subrange therebetween, for example, about 1 to 2, about 1 to 3, about 1 to 4, about 1 to 5, about 1 to 6, about 1 to 7, about 1 to 8, about 1 to 9, about 1 to 10, about 2 to 3, about 2 to 4, about 2 to 5, about 2 to 6, about 2 to 7, about 2 to 8, about 2 to 9, about 2 to 10, about 3 to 4, about 3 to 5, about 3 to 6, about 3 to 7, about 3 to 8, about 3 to 9, about 3 to 10, about 4 to 5, about 4 to 6, about 4 to 7, about 4 to 8, about 4 to 9, about 4 to 10, about 5 to 6, about 5 to 7, about 5 to 8, about 5 to 9, about 5 to 10, about 6 to 7, about 6 to 8, about 6 to 9, about 6 to 10, about 7 to 8, about 7 to 9, about 7 to 10, about 8 to 9, about 8 to 10, or about 9 to 10.

In certain embodiments, the DAR of the ADC composition described herein is about 3 to 9, for example, about 3.0 to 3.5, about 3.0 to 4.0, about 3.0 to 4.5, about 3.0 to 5.0, about 3.0 to 6.0, about 3.5 to 4.0, about 3.5 to 4.5, about 3.5 to 5.0, about 3.5 to 5.5, about 3.5 to 6.0, about 3.5 to 6.5, about 4.0 to 4.5, about 4.0 to 5.0, about 4.0 to 5.5, about 4.0 to 6.0, about 4.0 to 6.5, about 4.0 to 7.0, about 4.0 to 8.0, about 4.5 to 5.0, about 4.5 to 5.5, about 4.5 to 6.0, about 4.5 to 6.5, about 4.5 to 7.0, about 4.5 to 7.5, about 5.0 to 8.0, about 5.5 to 6.0, about 5.5 to 6.5, about 5.5 to 7.0, about 5.5 to 7.5, about 5.5 to 8.0, about 6.0 to 6.5, about 6.0 to 7.0, about 6.0 to 7.5, about 6.0 to 8.5, about 6.5 to 7.0, about 6.5 to 7.5, about 6.5 to 8.5, about 7.0 to 7.5.

In certain embodiments, the DAR of the ADC compositions described herein is about 3 to 8, for example, about 3.0 to 3.5, about 3.0 to 4.0, about 3.0 to 4.5, about 3.0 to 5.0, about 6.0 to 6.5, about 6.0 to 7.0, about 6.0 to 7.5, about 6.0 to 8.0, about 6.0 to 8.5, about 6.5 to 7.0, about 6.5 to 7.5, about 6.5 to 8.0, about 6.5 to 8.5, about 7.0 to 7.5, about 7.0 to 8.0, about 7.5 to 8.0.

In some embodiments, the DAR of the ADC compositions described herein is about 6.0 to 9.0, preferably about 6.0 to 8.0, such as about 6.0, about 6.01, about 6.02, about 6.03, about 6.04, about 6.05, about 6.06, about 6.07, about 6.08, about 6.09, about 6.1, about 6.11, about 6.12, about 6.13, about 6.14, about 6.15, about 6.16, about 6.17, about 6.18, about 6.19, about 6.2, about 6.21, about 6.22, about 6.23, about 6.24, about 6.25, about 6.26, about 6.27, about 6.28, about 6.29, about 6.3, about 6.31, about 6.32, about 6.33, about 6.34, about 6.35, about 6.36, about 6.37, about 6.38, about 6.39, about 6.4, about 6.41, about 6.42, about 6.43, about 6.44, about 6.45, about 6.46, about 6.47, about 6.48, about 6.49, about 6.5, about 6.51, about 6.52, about 6.53, about 6.54, about 6.55, about 6.56, about 6.57, about 6.58, about 6.59, about 6.6, about 6.61, about 6.62, about 6.63, about 6.64, about 6.65, about 6.66, about 6.67, about 6.68, about 6.69, about 6.7, about 6.71, about 6.72, about 6.73, about 6.74, about 6.75, about 6.76, about 6.77, about 6.78, about 6.79, about 6.8, about 6.81, about 6.82, about 6.83, about 6.84, about 6.85, about 6.86, about 6.87, about 6.88, about 6.89, about 6.9, about 6.91, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, about 7.1, about 7.11, about 7.12, about 7.13, about 7.14, about 7.15, about 7.16, about 7.17, about 7.18, about 7.19, about 7.2, about 7.21, about 7.22, about 7.23, about 7.24, about 7.25, about 7.26, about 7.27, about 7.28, about 7.29, about 7.3, about 7.31, about 7.32, about 7.33, about 7.34, about 7.35, about 7.36, about 7.37, about 7.38, about 7.39, about 7.4, about 7.41, about 7.42, about 7.43, about 7.44, about 7.45, about 7.46, about 7.47, about 7.48, about 7.49, about 7.5, about 7.51, about 7.52, about 7.53, about 7.54, about 7.55, about 7.56, about 7.57, about 7.58, about 7.59, about 7.6, about 7.61, about 7.62, about 7.63, about 7.64, about 7.65, about 7.66, about 7.67, about 7.68, about 7.69, about 7.7, about 7.71, about 7.72, about 7.73, about 7.74, about 7.75, about 7.76, about 7.77, about 7.78, about 7.79, about 7.8, about 7.81, about 7.82, about 7.83, about 7.84, about 7.85, about 7.86, about 7.87, about 7.88, about 7.89, about 7.9, about 7.91, about 7.92, about 7.93, about 7.94, about 7.95, about 7.96, about 7.97, about 7.98, about 7.99, about 8.0, about 8.01, about 8.02, about 8.03, about 8.04, about 8.05, about 8.06, about 8.07, about 8.08, about 8.09, about 8.1, about 8.11, about 8.12, about 8.13, about 8.14, about 8.15, about 8.16, about 8.17, about 8.18, about 8.19, about 8.2, about 8.21, about 8.22, about 8.23, about 8.24, about 8.25, about 8.26, about 8.27, about 8.28, about 8.29, about 8.3, about 8.31, about 8.32, about 8.33, about 8.34, about 8.35, about 8.36, about 8.37, about 8.38, about 8.39, about 8.4, about 8.41, about 8.42, about 8.43, about 8.44, about 8.45, about 8.46, about 8.47, about 8.48, about 8.49, about 8.5, about 8.51, about 8.52, about 8.53, about 8.54, about 8.55, about 8.56, about 8.57, about 8.58, about 8.59, about 8.6, about 8.61, about 8.62, about 8.63, about 8.64, about 8.65, about 8.66, about 8.67, about 8.68, about 8.69, about 8.7, about 8.71, about 8.72, about 8.73, about 8.74, about 8.75, about 8.76, about 8.77, about 8.78, about 8.79, about 8.8, about 8.81, about 8.82, about 8.83, about 8.84, about 8.85, about 8.86, about 8.87, about 8.88, about 8.89, about 8.9, about 8.91, about 8.92, about 8.93, about 8.94, about 8.95, about 8.96, about 8.97, about 8.98, about 8.99, about 9.0.

In certain embodiments, the composition is composed of an antibody-drug conjugate represented by Ab-[L′-D]ₓ. In certain embodiments, the DAR of the composition is 1 to 8, for example, 5 to 8, such as about 8.0, 7.89, 7.69, 7.91, 7.92, 7.85, 7.96, 7.42, 7.48, 7.91, 7.79, 7.99, 7.9. In certain embodiments, the antibody-drug conjugate with x of 8 accounts for more than 60%, more than 70%, more than 80%, or more than 90%.

### Pharmaceutical Composition

In another aspect, the present application provides a pharmaceutical composition, which comprises the compound or pharmaceutically acceptable salt thereof as described in any of the above aspects, or the conjugate as described in any of the above aspects, and one or more pharmaceutical excipients.

The compound or conjugate as described herein is typically generally formulated into a unit injectable form together with a pharmaceutically acceptable parenteral vehicle for parenteral use, such as push injection, intravenous injection, intratumoral injection, etc. Optionally, the antibody-drug conjugate with the desired purity and in the form of a lyophilized agent or solution is mixed with a pharmaceutically acceptable diluent, carrier, excipient or stabilizer (Remington's Pharmaceutical Sciences (1980) 16th edition, Osol, A. Ed.). The antibody-drug conjugate described herein or the pharmaceutical composition comprising the antibody-drug conjugate can be administered via any route suitable for the individual to be treated.

### Use

The present application provides a use of the compound or pharmaceutically acceptable salt thereof, conjugate, composition, or pharmaceutical composition as described above in the manufacture of a medicament for treating a cancer.

The present application provides the compound or pharmaceutically acceptable salt thereof, conjugate, composition, or pharmaceutical composition as described above, for use in treating a cancer.

The present application provides the compound or pharmaceutically acceptable salt thereof, conjugate, composition, or pharmaceutical composition as described above, for use in treating a solid tumor or hematological malignancy, which is, for example, gastric cancer, breast cancer, lung cancer (e.g., non-small cell lung cancer, specifically lung adenocarcinoma) or urothelial carcinoma.

The present application provides a method for treating cancer, the method comprising administering to an individual in need thereof a therapeutically effective amount of the compound or pharmaceutically acceptable salt thereof, conjugate, composition, or pharmaceutical composition as described above.

In some embodiments, the cancer is selected from the group consisting of solid tumors or hematological malignancies, for example, gastric cancer, breast cancer, lung cancer (e.g., non-small cell lung cancer, specifically lung adenocarcinoma) or urothelial carcinoma.

In some embodiments, the antibody-drug conjugate, compound, drug-linker or pharmaceutical composition containing the same is sufficient (e.g., in a subject) to:
(1) inhibit the proliferation of cells (e.g., tumor cells);
(2) inhibit tumor growth;
(3) induce and/or increase antibody-dependent cellular cytotoxicity activity;
(4) inhibit signal transduction;
(5) prevent and/or treat cancer; or
(6) any combination of the above (1) to (5).

In some embodiments, the cancer disease is selected from the group consisting of solid tumors or hematological malignancies, for example, gastric cancer, breast cancer, lung cancer (e.g., non-small cell lung cancer, specifically lung adenocarcinoma) or urothelial carcinoma.

For all technical features disclosed in the description, such as the definitions of groups, etc., except for mutually exclusive technical features, all embodiments thereof can be combined in any way to obtain different general formula ranges or specific schemes. These ranges and schemes are all within the scope of the present invention.

### Definition

Unless otherwise defined below, the meanings of all technical and scientific terms used herein are intended to be the same as those generally understood by a skilled person in the art. References to the technology used herein is shall mean the technology generally recognized in the art, including technological modifications or equivalent substitutions apparent to those skilled in the art. In addition, the laboratory procedures in genomics, nucleic acid chemistry, molecular biology, etc. used herein are all routine steps widely adopted in the respective fields. Although it is believed that the following terms are well understood by those skilled in the art, the following definitions are still provided herein to better explain the present invention.

The term “antibody” refers to an immunoglobulin molecule that is typically composed of two pairs of polypeptide chains, each pair having one light chain (LC) and one heavy chain (HC). Antibody light chains can be categorized into κ (kappa) and λ (lambda) types. Heavy chains may be categorized into μ, δ, γ, α or ε, with corresponding antibody isotypes defined as IgM, IgD, IgG, IgA and IgE, respectively. In the light chain and heavy chain, the variable region and the constant region are connected by a “J” region of about 12 or more amino acids, and the heavy chain also contains a “D” region of about 3 or more amino acids. Each heavy chain consists of a heavy chain variable region (V_{H}) and a heavy chain constant region (CH). The heavy chain constant region consists of 3 domains (CH1, CH2 and CH3). Each light chain consists of a light chain variable region (V_{L}) and a light chain constant region (CL). The light chain constant region consists of one domain CL. The constant domain does not directly participate in the binding of antibodies to antigens, but exhibits a variety of effector functions, such as mediating the binding of immunoglobulins to host tissues or factors, including various cells of the immune system (e.g., effector cells) and the first component (C1q) of the classical complement system. The V_{H} and V_{L} regions may also be subdivided into highly variable regions (called complementarity determining regions (CDRs)), interspersed with more conserved regions called framework regions (FRs). Each of VH and VL consists of three CDRs and four FRs arranged from the amino terminus to the carboxyl terminus in the following order: FR1, CDR1, FR2, CDR2, FR3, CDR3, FR4. The variable regions (V_{H} and V_{L}) of each heavy chain/light chain pair form antigen binding sites, respectively. Amino acid numbering in each region or domain may follow various numbering systems known in the art.

The term “antibody” further includes variants where the heavy chain constant region has a C-terminal lysine, lacks a C-terminal lysine, or lacks a C-terminal glycine-lysine dipeptide. It also includes variants where the N-terminal amino acid of the antibody variable region is cyclized to pyroglutamate. Thus, in a composition comprising the disclosed antibodies, each antibody may independently have a C-terminal lysine, lack a C-terminal lysine or C-terminal glycine-lysine, and/or have a N-terminal glutamine or glutamic acid, or pyroglutamate resulting from cyclization of the N-terminal amino acid.

The term “complementarity determining region” or “CDR” refers to the amino acid residues in the variable region of an antibody that are responsible for antigen binding. The variable regions of the heavy chain and light chain each contains three CDRs, designated CDR1, CDR2, and CDR3. The precise boundaries of these CDRs may be defined according to various numbering systems known in the art, for example according to the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883), the IMGT numbering system (Lefranc et al., Dev. Comparat. Immunol. 27:55-77, 2003), or the AbM numbering system (Martin A C R, Cheetham J C, Rees A R (1989) Modelling antibody hypervariable loops: A combined algorithm. Proc Natl Acad Sci USA 86:9268-9272). For a given antibody, a person skilled in the art will readily identify the CDRs defined by each numbering system. Furthermore, the correspondence between different numbering systems is well known to a person skilled in the art (e.g., see Lefranc et al., Dev. Comparat. Immunol. 27:55-77, 2003).

In the present invention, the CDRs comprised in an antibody or antigen-binding fragment thereof may be determined according to various numbering systems known in the art, such as by Kabat, Chothia, IMGT or AbM numbering systems. In certain embodiments, the CDRs comprised in an antibody or antigen-binding fragment thereof are determined by the Chothia numbering system.

The following general rules disclosed in www.bioinf.org.uk: Professor Andrew C. R. Martin's research group may be used to define CDRs in an antibody sequence that includes those amino acids that specifically interact with the amino acids comprising the epitope in the antigen to which the antibody binds. There are rare examples where these generally constant features do not occur; however, the Cys residues are the most conserved features.

| Loop | Kabat | AbM | Chothia ¹ | Contact ² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26-- | H30--H35B | H26--H35B |
| | (Kabat numbering | | H32 . . . 34 | | |
| | system)³ | | | | |
| H1 | H31--H35 | H26--H35 | H26--H32 | H30--H35 | H26--H33 |
| | (Chothia numbering | | | | |
| | system) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹ Some of these numbering systems (particularly for Chothia numbering system) vary depending on the individual publication examined.
² Any of the numbering system can be used for these CDR definitions, except the Contact numbering system uses the Chothia or Martin (Enhanced Chothia) definition.
³The end of the Chothia CDR-H1 loop when numbered using the Kabat numbering system varies between H32 and H34 depending on the length of the loop. (This is because the Kabat numbering system places insertions at H35A and H35B).
If neither H35A nor H35B is present, the loop ends at H32;
If only H35A is present, the loop ends at H33;
If both H35A and H35B are present, the loop ends at H34.

The entire amino acid sequence of V_{H} is commonly numbered according to Kabat, while the three CDRs within the variable region may be defined according to any of the aforementioned numbering systems. In particular embodiments, the numbering of the amino acid positions in V_{H} may be sequential beginning with amino acid position 1 continuing sequentially to the end of the sequence or according to Kabat. Unless specified otherwise, the amino acid positions in V_{H} and V_{L} described herein are defined according to sequential numbering.

The numbering of amino acid positions in the heavy chain constant region may be sequential beginning with amino acid position 1 and continuing sequentially to the end of the sequence or according to Eu numbering. The IgG1 heavy chain constant region amino acid sequence has 330 amino acids sequentially numbered 1 to 330. The corresponding sequence numbered according to Eu begin with position number 118 and ends with position number 447. Unless specified otherwise, the amino acid positions in the heavy and light chains described herein are defined according to sequential numbering.

The term “framework region” or “FR” residues refers to those amino acid residues in the antibody variable region other than the CDR residues as defined above.

The term “antibody” is not limited to any particular method of producing the antibody. This includes, for example, recombinant antibodies, monoclonal antibodies, and polyclonal antibodies. The antibodies may be of different isotypes, for example, IgG (e.g., IgG1, IgG2, IgG3 or IgG4 subtypes), IgA1, IgA2, IgD, IgE or IgM antibodies.

The term “antigen-binding fragment” of an antibody refers to a polypeptide of a fragment of an antibody, such as a polypeptide of a fragment of a full-length antibody, which retains the ability to specifically bind to the same antigen bound by the full-length antibody and/or competes with the full-length antibody for specific binding to the antigen, which is also referred to as an “antigen-binding portion”. See generally, Fundamental Immunology, Ch. 7 (Paul, W., ed., 2nd ed., Raven Press, N.Y. (1989), which is incorporated herein by reference in its entirety for all purposes. An antigen-binding fragment of an antibody may be produced by a recombinant DNA technique or by an enzymatic or chemical cleavage of the intact antibody. Non-limiting examples of antigen-binding fragment include Fab fragment, Fab′ fragment, F(ab)′₂ fragment, F(ab)′₃ fragment, Fd, Fv, scFv, di-scFv, (scFv)₂, disulfide-stabilized Fv protein (“dsFv”), single domain antibody (sdAb, nanobody), and a polypeptide that comprises at least a portion of an antibody sufficient to confer specific antigen binding ability to the polypeptide. Engineered antibody variants are reviewed in Holliger et al., 2005; Nat Biotechnol, 23: 1126-1136.

The term “Fd” refers to an antibody fragment consisting of the V_{H} and CH1 domains; the term “dAb fragment” refers to an antibody fragment consisting of the V_{H} domain (Ward et al., Nature 341:544 546 (1989)); the term “Fab fragment” refers to an antibody fragment consisting of the VL, VH, CL and CH1 domains; the term “F(ab′)₂ fragment” refer to an antibody fragment comprising two Fab fragments connected by a disulfide bridge on the hinge region; the term “Fab′ fragment” refers to a fragment obtained after reducing the disulfide bonds connecting the two heavy chain fragments in the F(ab′)₂ fragment, which consists of a complete light chain and the Fd fragment of the heavy chain (consisting of the V_{H} and CH1 domains).

The term “Fv” refers to an antibody fragment consisting of the V_{L} and V_{H} domains of a single arm of an antibody. The Fv fragment is generally considered to be the smallest antibody fragment that may form a complete antigen binding site. It is generally believed, six CDRs confer antigen-binding specificity to an antibody. However, even a single variable region (e.g., an Fd fragment, which contains only three CDRs specific for an antigen) is able to recognize and bind to an antigen, although its affinity may be lower than that of the complete binding site.

The term “Fc” refers to an antibody fragment formed by disulfide bonding of the second and third constant regions of the first heavy chain of an antibody to the second and third constant regions of the second heavy chain. The Fc fragment of an antibody has a variety of different functions but is not involved in antigen binding.

The term “scFv” refers to a single polypeptide chain containing V_{L} and V_{H} domains connected by a linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, Roseburg and Moore, eds., Springer-Verlag, New York, pp. 269-315 (1994)). Such scFv molecules may have a general structure: NH₂-V_{L}-linker-V_{H}—COOH or NH₂-V_{H}-linker-V_{L}-COOH. A suitable prior art peptide linker consists of the repeated GGGGS (SEQ ID NO: 48) amino acid sequence or its variants. For example, a linker having the amino acid sequence (GGGGS)₄ (SEQ ID NO: 49) may be used, but variants thereof may also be used (Holliger et al. (1993), Proc. Natl. Acad. Sci. USA 90: 6444-6448). Other linkers that may be used in the present invention are described by Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol. In some cases, a disulfide bond may also exist between the V_{H} and V_{L} of the scFv. In certain embodiments, the V_{H} and V_{L} domains may be positioned relative to each other in any suitable arrangement. For example, a scFv may comprise NH₂—VH—VH—COOH, NH₂—VL-VL-COOH.

The term “single-domain antibody (sdAb)” has the meaning commonly understood by those skilled in the art, which refers to an antibody fragment composed of a single monomeric variable antibody domain (e.g., a single heavy chain variable region) that retains the ability to specifically bind to the same antigen bound by a full-length antibody (Holt, L. et al., Trends in Biotechnology, 21(11):484-490, 2003). Single-domain antibody is also called nanobody.

The above-mentioned antibody fragments all retain the ability to specifically bind to the same antigen bound by a full-length antibody, and/or compete with the full-length antibody for specific binding to the antigen.

As used therein, unless the context clearly indicates otherwise, when referring to the term “antibody”, it includes not only an intact antibody but also an antigen-binding fragment of the antibody.

An antigen-binding fragment of antibody (e.g., the above-mentioned antibody fragment) may be obtained from a given antibody (e.g., the antibody provided by the present invention) using a conventional technique known to a skilled person in the art (e.g., recombinant DNA technology or enzymatic or chemical cleavage method), and the antigen-binding fragment of antibody may be screened for specificity in the same manner as for the intact antibody.

The term “murine antibody” refers to an antibody obtained by the following method: fusing B cells of immunized mice with myeloma cells, screening for murine hybrid fusion cells that may both proliferate indefinitely and secrete antibodies, followed by screening, antibody preparation and antibody purification; or refers to an antibody secreted by plasma cells formed by differentiation and proliferation of B cells after an antigen invades mice.

The term “humanized antibody” refers to a non-human antibody modified by genetic engineering, of which amino acid sequence is modified to increase the homology with the sequence of a human antibody. Generally speaking, all or part of the CDR region of a humanized antibody comes from a non-human antibody (donor antibody), and all or part of the non-CDR region (e.g., variable region FR and/or constant region) comes from a human immunoglobulin (recipient antibody). The humanized antibody generally retains the expected properties of the donor antibody, including, but not limited to, antigen specificity, affinity, reactivity, ability to increase immune cell activity, ability to enhance immune response, etc. The donor antibody may be a mouse, rat, rabbit or non-human primate (e.g., cynomolgus monkey) antibody with the expected properties (e.g., antigen specificity, affinity, reactivity, ability to increase immune cell activity and/or ability to enhance immune response).

The term “identity” is used to refer to a match of sequences between two polypeptides or between two nucleic acids. When a position in both sequences being compared is occupied by the same base or amino acid monomer subunit (e.g., a position in each of two DNA molecules is occupied by adenine, or a position in each of two polypeptides is occupied by lysine), then the molecules are identical at that position. “Percent identity” between two sequences is a function of the number of matching positions common to the two sequences divided by the number of positions compared×100. For example, if 6 out of 10 positions of two sequences match, then the two sequences are 60% identical. For example, the DNA sequences CTGACT and CAGGTT share 50% identity (3 positions match out of 6 total positions). Typically, comparisons are made when two sequences are aligned to yield maximum identity. Such alignments can be achieved using, for example, the method of Needleman et al. (1970) J. Mol. Biol. 48: 443-453, which can be conveniently performed by computer programs such as the Align program (DNAstar, Inc.). The algorithm of E. Meyers and W. Miller (Comput. Appl Biosci., 4:11-17 (1988)) which has been incorporated into the ALIGN program (version 2.0), can be used to determine the percent identity between two amino acid sequences by using the PAM120 weight residue table, a gap length penalty of 12, and a gap penalty of 4. Alternatively, the algorithm of Needleman and Wunsch (J MoI Biol. 48:444-453 (1970)) which has been integrated into the GAP program of the GCG software package (available at www.gcg.com) can be used to determine the percent identity between two amino acid sequences by using the Blossum 62 matrix or PAM250 matrix with a gap weight of 16, 14, 12, 10, 8, 6, or 4, and a length weight of 1, 2, 3, 4, 5, or 6.

The term “conservative substitution” refers to an amino acid substitution that does not adversely affect or change the expected properties of the protein/polypeptide comprising the amino acid sequence. For example, a conservative substitution may be introduced by a standard technique known in the art such as site-directed mutagenesis and PCR-mediated mutagenesis. Conservative amino acid substitutions include substitutions in which amino acid residues are substituted with amino acid residues having similar side chains, such as substitutions with residues that are physically or functionally similar to the corresponding amino acid residues (e.g., having similar size, shape, charge, chemical properties, including the ability to form covalent bonds or hydrogen bonds, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Thus, it is preferred to replace a corresponding amino acid residue with another amino acid residue from the same side chain family. Methods for identifying conservative substitutions of amino acids are well known in the art (see, for example, Brummell et al., Biochem. 32:1180-1187 (1993); Kobayashi et al. Protein Eng. 12(10):879-884 (1999); and Burks et al. Proc. Natl Acad. Set USA 94:412-417 (1997), which are incorporated herein by reference).

The twenty conventional amino acids used herein are written in accordance with conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, amino acids are generally represented by single-letter and three-letter abbreviations known in the art. For example, alanine may be represented by A or Ala.

The term “linker” refers to a structural fragment that connects a cytotoxic drug to an antibody or antigen-binding fragment thereof. For example, it refers to the -M-L-E- structural fragment in the formula Ab-[M-L-E-D]ₓ.

The term “drug-linker” refers to a structure of the cytotoxic drug and linker described in the present invention before being connected to an antibody or antigen-binding fragment thereof. For example, “drug-linker” refers to M′-L-E-D, wherein M′ is the structural form of M before covalently connected to an antibody or antigen-binding fragment thereof. “Drug-linker” is covalently connected to an antibody or antigen-binding fragment thereof to obtain the antibody-drug conjugate described in the present application.

The “drug-linker” also includes all pharmaceutically acceptable isotopically labeled compounds thereof, which are the same as the “drug-linker” compound of the present invention, except that one or more atoms are replaced by atoms having the same atomic number but an atomic mass or mass number different from the atomic mass or mass number that predominates in nature. Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O, and ¹⁸O); and isotopes of sulfur (e.g., ³⁵S).

The terms “include,” “comprises,” “have,” “contain,” or “involve,” and other variations thereof as used herein, are inclusive or open-ended and do not exclude other unlisted elements or method steps.

The term “alkyl” refers to a group, such as “C₁₋₂₀ alkyl”, “C₁₋₁₀ alkyl”, “C₁₋₆ alkyl”, “C₁₋₄ alkyl”, “C₁₋₃ alkyl”, etc., which is obtained by removing one hydrogen atom from a straight or branched hydrocarbonyl. Its specific examples include, but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “alkylene” refers to a group obtained by removing two hydrogen atoms from a straight or branched hydrocarbonyl, such as “C₁₋₆ alkylene”, “C₁₋₅ alkylene”, “C₁₋₄ alkylene”, “C₁₋₃ alkylene”, and its specific examples include but are not limited to: methylene, ethylene, propylene, butylene, etc.

The term “aryl” refers to an unsaturated carbocyclic group with a conjugated R electron system, such as “6- to 10-membered aryl”, and its specific examples include but are not limited to: phenyl, naphthyl.

The term “heteroaryl” refers to an unsaturated group with a conjugated T1 electron system composed of ring atoms, in which at least one (e.g. 1, 2, 3 or 4) of the ring atoms is a heteroatom, such as N, O and S, wherein the nitrogen atom is optionally quaternized, and the nitrogen and sulfur heteroatoms are optionally oxidized. For example, it can be “5- to 12-membered heteroaryl”, “5- to 11-membered heteroaryl”, “5- to 10-membered heteroaryl”, “5- to 9-membered heteroaryl”, “5- to 6-membered heteroaryl”, and its specific examples include but are not limited to: phenyl, naphthyl, furanyl, pyrrolyl, thienyl, pyrazolyl, imidazolyl, thiazolyl, isothiazolyl, thiadiazolyl, oxazolyl, isoxazolyl, oxadiazolyl, pyridyl, pyrimidinyl, pyridazinyl, pyrazinyl, indazolyl, indolyl, quinolyl, isoquinolyl.

The term “heterocyclyl” refers to a saturated or partially unsaturated cyclic group composed of ring atoms, in which at least one of the ring atoms (e.g., 1, 2, 3, 4 or 5 heteroatoms) is a heteroatom, such as N, O and S, wherein the nitrogen atom is optionally quaternized, the nitrogen and sulfur heteroatoms are optionally oxidized, and the carbon atom is optionally oxidized. The heterocyclyl comprises monocyclic ring, bicyclic ring or polycyclic ring, including spirocyclic ring, fused cyclic ring or bridged cyclic ring. The term “nitrogen-containing heterocyclyl” means that at least one heteroatom is N, such as “5- to 12-membered nitrogen-containing heterocyclyl”, “5- to 9-membered nitrogen-containing heterocyclyl”, “9- to 12-membered nitrogen-containing heterocyclyl”, and its specific examples include but are not limited to: pyrrolidinyl, piperidinyl, piperazinyl, morpholinyl, 1,2,3,4-tetrahydroquinolinyl.

The term “isotopically labeled compound” means that the compound is identical in structure to the compound of the present invention except that one or more atoms are replaced by atoms having the same atomic number but an atomic mass or mass number different from the atomic mass or mass number that predominates in nature. Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O and ¹⁸O); and isotopes of sulfur (e.g., ³⁵S).

As used herein, the term “suitable substituent” refers to a modification that a person skilled in the art can make to a compound according to the need of the compound substituent. “Suitable substituent” includes oxo (═O), halogen, cyano, NR⁸R⁹, carboxyl, sulfhydryl group, hydroxyl, ester (e.g., —C₁₋₆ alkyl-C(═O)—OC₁₋₆ alkyl), C₁₋₆ alkyl, C₂₋₆ alkenyl, C₂₋₆ alkynyl, C₁₋₆ alkyl-O—C₁₋₆ alkyl, C₁₋₆ haloalkyl, C₁₋₆ alkoxy, C₃₋₆ cycloalkyl, 3- to 10-membered heterocyclyl, 5- to 10-membered heteroaryl, C₆₋₁₀ aryl, benzyl, hydroxy-substituted benzyl, indolylmethylene and C₁₋₆ haloalkoxy, wherein R⁸, R⁹ are each independently selected from the group consisting of H, C₁₋₆ alkyl, C₃₋₆ cycloalkyl, 3- to 10-membered heterocyclyl, 5- to 10-membered heteroaryl, C₆₋₁₀ aryl, C₁₋₆ alkoxy, C₁₋₆ haloalkyl, C₁₋₆ haloalkoxy, halogen, hydroxyl, carboxyl and ester (e.g. —C₁₋₆ alkyl-C(═O)—OC₁₋₆ alkyl).

The term “substituted” means that one or more (e.g. 1, 2, 3, 4 or 5) hydrogen atoms on the specified compound or structural fragment are replaced by a substituent, provided that the normal valence of the specified atom in the current situation is not exceeded and the substitution forms a stable compound. Combinations of substituents and/or variables are permissible only if they yield stable compounds. In some embodiments, the substituent is each independently composed of one or more of the following structures: —O—, —S—, —NR′—, halogen, —CN, —OH, —NH₂, —NO₂, —CN, ═O, C₁-C₆ alkyl(ene), C₁-C₆ haloalkyl(ene), C₁-C₆ alkoxy, C₂-C₆ alkenyl(ene), C₂-C₆ alkynyl(ene), C₃-C₈ cycloalkyl(ene), 3- to 8-membered heterocyclyl(ene), C₆-C₁₀ aryl(ene) and 5- to 10-membered heteroaryl(ene), etc. In some embodiments, the substituent is each independently composed of one or more of the following structures: NR⁸R⁹, —O—, —S—, —NR′—, halogen, —CN, —OH, —SH, —NH₂, —NO₂, —C(O)—, —CN, ═O, C₁-C₆ alkyl(ene), C₁-C₆ haloalkyl(ene), C₁-C₆ alkoxy, C₂-C₆ alkenyl(ene), C₂-C₆ alkynyl(ene), C₃-C₈ cycloalkyl(ene), 3- to 10-membered heterocyclyl(ene), C₆-C₁₀ aryl(ene), and 5- to 10-membered heteroaryl(ene), etc., wherein R⁸, R⁹ and R′ are as defined above. For example, the substituents may be suitable substituents as described above.

If a functional group or structural fragment is described as “substituted or unsubstituted”, the functional group or structural fragment may be (1) unsubstituted or (2) substituted.

As used herein, the term “one or more” means 1 or more than 1, such as 2, 3, 4, 5, 6, 7, 8, 9, or 10, under reasonable conditions.

Unless otherwise specified, as used herein, the site for attachment of a substituent may be from any suitable position of the substituent.

Whether or not explicitly indicated, all numerical values in the present application are modified by the term “about”. The term “about” refers to a range of ±20% of the stated value, ±10% of the stated value, preferably ±5% of the stated value, and more preferably ±2% of the stated value.

### BRIEF DESCRIPTION OF THE DRAWINGS

FIG. 1 shows the anti-tumor effects of anti-human HER2 antibody-drug conjugate ADCs on NCI-N87 cell subcutaneous tumor-bearing mouse model.

FIG. 2 shows the anti-tumor effects of anti-human HER2 antibody-drug conjugate ADCs on NCI-N87 cell subcutaneous tumor-bearing mouse model.

FIG. 3 shows the changes in body weight of each group of mice in the human gastric cancer cell NCI-N87 CDX model.

FIG. 4 shows the efficacy results of anti-human HER2 antibody-drug conjugate ADCs on NCI-N87 cell subcutaneous tumor-bearing mouse model.

FIG. 5 shows the efficacy results of anti-human HER2 antibody-drug conjugate ADCs on NCI-N87 cell subcutaneous tumor-bearing mouse model.

FIG. 6 shows the changes in body weight of each group of mice in the human gastric cancer cell NCI-N87 CDX model.

FIG. 7 shows the efficacy results of anti-human HER2 antibody-drug conjugate ADCs on the NCI-N87 cell subcutaneous tumor-bearing mouse model.

FIG. 8 shows the changes in body weight of each group of mice in the human gastric cancer cell NCI-N87 CDX model.

FIG. 9 shows the efficacy results of anti-human HER2 antibody-drug conjugate ADCs on the JIMT-1 cell subcutaneous tumor-bearing mouse model.

FIG. 10 shows the changes in body weight of each group of mice in the human breast cancer cell JIMT-1 CDX model.

FIG. 11 shows the efficacy results of anti-human HER2 antibody-drug conjugate ADCs on the HCC827 cell subcutaneous tumor-bearing mouse model.

FIG. 12 shows the changes in body weight of each group of mice in the human non-small cell lung cancer HCC827 CDX model.

FIG. 13 shows the efficacy results of anti-human HER2 antibody-drug conjugate ADCs on the NCI-N87 cell subcutaneous tumor-bearing mouse model.

FIG. 14 shows the efficacy results of anti-human HER2 antibody-drug conjugate ADCs on the NCI-N87 cell subcutaneous tumor-bearing mouse model.

FIG. 15 shows the changes in the body weight of each group of mice in the human gastric cancer cell NCI-N87 CDX model.

FIG. 16 shows the efficacy results of anti-human HER2 antibody-drug conjugate ADCs on the NCI-N87 cell subcutaneous tumor-bearing mouse model.

FIG. 17 shows the efficacy results of anti-human HER2 antibody-drug conjugate ADCs on the NCI-N87 cell subcutaneous tumor-bearing mouse model.

FIG. 18 shows the changes in the body weight of each group of mice in the human gastric cancer cell NCI-N87 CDX model.

FIG. 19 shows the efficacy results of anti-human HER2 antibody-drug conjugate ADCs on the JIMT-1 cell subcutaneous tumor-bearing mouse model.

FIG. 20 shows the efficacy results of anti-human HER2 antibody-drug conjugate ADCs on JIMT-1 cell subcutaneous tumor-bearing mouse model.

FIG. 21 shows the changes in body weight of each group of mice in the human breast cancer cell JIMT-1 CDX model.

FIG. 22 shows the efficacy results of anti-human HER2 antibody-drug conjugate ADCs on NCI-N87 cell subcutaneous tumor-bearing mouse model.

FIG. 23 shows the efficacy results of ani-human HER2 antibody-drug conjugate ADCs on NCI-N87 cell subcutaneous tumor-bearing mouse model.

FIG. 24 shows the efficacy results of anti-human HER2 antibody-drug conjugate ADCs on NCI-N87 cell subcutaneous tumor-bearing mouse model.

FIG. 25 shows the changes in body weight of each group of mice in the human gastric cancer cell NCI-N87 CDX model.

### SPECIFIC MODELS FOR CARRYING OUT THE PRESENT INVENTION

The present invention is further illustrated through the description of specific embodiments, but this is not a limitation of the present invention. Based on the teachings of the present invention, those skilled in the art can make various modifications or improvements without departing from the basic idea and scope of the present invention.

The information of the sequences involved in the present invention is described in the following table:

| SEQ | | |
|---|---|---|
| ID | | |
| NO: | Sequence Name | Sequence Information |
| 1 | Trastuzumab VH amino acid | EVQLVESGGGLVQPGGSLRLSCAASGFNIKDTYIHWVRQ |
| | sequence | APGKGLEWVARIYPTNGYTRYADSVKGRFTISADTSKNT |
| | | AYLQMNSLRAEDTAVYYCSRWGGDGFYAMDYWGQGT |
| | | LVTVSS |
| 2 | Trastuzumab VL amino acid | DIQMTQSPSSLSASVGDRVTITCRASQDVNTAVAWYQQK |
| | sequence | PGKAPKLLIYSASFLYSGVPSRFSGSRSGTDFTLTISSLQPE |
| | | DFATYYCQQHYTTPPTFGQGTKVEIK |
| 3 | Pertuzumab VH amino acid | EVQLVESGGGLVQPGGSLRLSCAASGFTFTDYTMDWVR |
| | sequence | QAPGKGLEWVADVNPNSGGSIYNQRFKGRFTLSVDRSK |
| | | NTLYLQMNSLRAEDTAVYYCARNLGPSFYFDYWGQGTL |
| | | VTVSS |
| 4 | Pertuzumab VL amino acid | DIQMTQSPSSLSASVGDRVTITCKASQDVSIGVAWYQQK |
| | sequence | PGKAPKLLIYSASYRYTGVPSRFSGSGSGTDFTLTISSLQP |
| | | EDFATYYCQQYYIYPYTFGQGTKVEIK |
| 5 | Trastuzumab Chothia CDR | GFNIKDT |
| | H1 | |
| 6 | Trastuzumab Chothia CDR | YPTNGY |
| | H2 | |
| 7 | Trastuzumab | WGGDGFYAMDY |
| | Chothia/kabat/AbM CDR H3 | |
| 8 | Trastuzumab | RASQDVNTAVA |
| | Chothia/Kabat/AbM CDR L1 | |
| 9 | Trastuzumab | SASFLYS |
| | Chothia/Kabat/AbM CDR L2 | |
| 10 | Trastuzumab | QQHYTTPPT |
| | Chothia/Kabat/IMGT/AbM | |
| | CDR L3 | |
| 11 | Trastuzumab Kabat CDR H1 | DTYIH |
| 12 | Trastuzumab Kabat CDR H2 | RIYPTNGYTRYADSVKG |
| 13 | Trastuzumab IMGT CDR H1 | GFNIKDTY |
| 14 | Trastuzumab IMGT CDR H2 | IYPTNGYT |
| 15 | Trastuzumab IMGT CDR H3 | SRWGGDGFYAMDY |
| 16 | Trastuzumab IMGT CDR L1 | QDVNTA |
| 17 | Trastuzumab IMGT CDR L2 | SAS |
| 18 | Trastuzumab AbM CDR H1 | GFNIKDTYIH |
| 19 | Trastuzumab AbM CDR H2 | RIYPTNGYTR |
| 20 | Pertuzumab Chothia CDR | GFTFTDY |
| | H1 | |
| 21 | Pertuzumab Chothia CDR | NPNSGG |
| | H2 | |
| 22 | Pertuzumab | NLGPSFYFDY |
| | Chothia/Kabat/AbM CDR | |
| | H3 | |
| 23 | Pertuzumab | KASQDVSIGVA |
| | Chothia/Kabat/AbM CDR L1 | |
| 24 | Pertuzumab | SASYRYT |
| | Chothia/Kabat/AbM CDR L2 | |
| 25 | Pertuzumab | QQYYIYPYT |
| | Chothia/Kabat/IMGT/AbM | |
| | CDR L3 | |
| 26 | Pertuzumab Kabat CDR H1 | DYTMD |
| 27 | Pertuzumab Kabat CDR H2 | DVNPNSGGSIYNQRFKG |
| 28 | Pertuzumab IMGT CDR H1 | GFTFTDYT |
| 29 | Pertuzumab IMGT CDR H2 | VNPNSGGS |
| 30 | Pertuzumab IMGT CDR H3 | ARNLGPSFYFDY |
| 31 | Pertuzumab IMGT CDR L 1 | QDVSIG |
| 32 | Pertuzumab IMGT CDR L2 | SAS |
| 33 | Pertuzumab AbM CDR H1 | GFTFTDYTMD |
| 34 | Pertuzumab AbM CDR H2 | DVNPNSGGSI |
| 35 | Heavy chain constant region | ASTKGPSVFPLAPSSKSTSGGTAALGCLVKDYFPEPVTVS |
| | amino acid sequence of | WNSGALTSGVHTFPAVLQSSGLYSLSSVVTVPSSSLGTQT |
| | Trastuzumab or Pertuzumab | YICNVNHKPSNTKVDKKVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDW |
| | | LNGKEYKCKVSNKALPAPIEKTISKAKGQPREPQVYTLPP |
| | | SREEMTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 36 | Light chain constant region | RTVAAPSVFIFPPSDEQLKSGTASVVCLLNNFYPREAKVQ |
| | amino acid sequence of | WKVDNALQSGNSQESVTEQDSKDSTYSLSSTLTLSKADY |
| | Trastuzumab or Pertuzumab | EKHKVYACEVTHQGLSSPVTKSFNRGEC |
| 37 | Heavy chain amino acid | EVQLVESGGGLVQPGGSLRLSCAASGFNIKDTYIHWVRQ |
| | sequence of Trastuzumab | APGKGLEWVARIYPTNGYTRYADSVKGRFTISADTSKNT |
| | | AYLQMNSLRAEDTAVYYCSRWGGDGFYAMDYWGQGT |
| | | LVTVSSASTKGPSVFPLAPSSKSTSGGTAALGCLVKDYFP |
| | | EPVTVSWNSGALTSGVHTFPAVLQSSGLYSLSSVVTVPSS |
| | | SLGTQTYICNVNHKPSNTKVDKKVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLTV |
| | | LHQDWLNGKEYKCKVSNKALPAPIEKTISKAKGQPREPQ |
| | | VYTLPPSREEMTKNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| 38 | Light chain amino acid | DIQMTQSPSSLSASVGDRVTITCRASQDVNTAVAWYQQK |
| | sequence of Trastuzumab | PGKAPKLLIYSASFLYSGVPSRFSGSRSGTDFTLTISSLQPE |
| | | DFATYYCQQHYTTPPTFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |
| 39 | Heavy chain amino acid | EVQLVESGGGLVQPGGSLRLSCAASGFTFTDYTMDWVR |
| | sequence of Pertuzumab | QAPGKGLEWVADVNPNSGGSIYNQRFKGRFTLSVDRSK |
| | | NTLYLQMNSLRAEDTAVYYCARNLGPSFYFDYWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSSS |
| | | LGTQTYICNVNHKPSNTKVDKKVEPKSCDKTHTCPPCPA |
| | | PELLGGPSVFLFPPKPKDTLMISRTPEVTCVVVDVSHEDP |
| | | EVKFNWYVDGVEVHNAKTKPREEQYNSTYRVVSVLTVL |
| | | HQDWLNGKEYKCKVSNKALPAPIEKTISKAKGQPREPQV |
| | | YTLPPSREEMTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSPGK |
| 40 | Light chain amino acid | DIQMTQSPSSLSASVGDRVTITCKASQDVSIGVAWYQQK |
| | sequence of Pertuzumab | PGKAPKLLIYSASYRYTGVPSRFSGSGSGTDFTLTISSLQP |
| | | EDFATYYCQQYYIYPYTFGQGTKVEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 41 | Short peptide | GGFG |
| 42 | Short peptide | GGVA |
| 43 | Short peptide | GFLG |
| 44 | Short peptide | EAAA |
| 45 | Short peptide | GGGGG |
| 46 | Short peptide | DGGFG |
| 47 | Short peptide | EGGFG |
| 48 | Linker subunit | GGGGS |
| 49 | (GGGGS)₄ | GGGGGGGGSGGGGSGGGGS |

The abbreviations used herein have the following meanings:

| Abbreviation | Meaning | Abbreviation | Meaning |
|---|---|---|---|
| HATU | N,N,N′,N′-Tetramethyl-O-(7- | EDCI | 1-(3-Dimethylaminopropyl)-3- |
| | azabenzotriazol-1-yl)uronium | | ethylcarbodiimide hydrochloride |
| | hexafluorophosphate | | |
| EEDQ | 2-Ethoxy-1-ethoxycarbonyl- | HOBt | 1-hydroxybenzotriazole |
| | 1,2-dihydroquinoline | | |
| PyBOP | 1H-Benzotriazol-1- | DMTMM | 4-(4,6-Dimethoxytriazin-2-yl)- |
| | yloxytripyrrolidinyl | | 4-methylmorpholine |
| | hexafluorophosphate | | hydrochloride |
| DIPEA | Diisopropylethylamine | DBU | 1,8-Diazobispiro[5.4.0]undec-7- |
| | | | ene |
| DMAP | 4-Dimethylaminopyridine | DCC | N,N′-dicyclohexylcarbodiimide |
| HOSu | N-hydroxysuccinimide | PPTS | Pyridinium p-toluenesulfonate |
| TsOH | p-Toluenesulfonic acid | TFA | Trifluoroacetic acid |
| AcOH | Acetic acid | LiOH•H₂O | Lithium hydroxide monohydrate |
| NaHCO₃ | Sodium bicarbonate | Pd(PPh₃)₄ | Tetratriphenylphosphine |
| | | | palladium |
| Pb(OAc)₄ | Lead tetraacetate | MMAE | Monomethyl auristatin E |
| AE | Auristatin E | Cit | Citrulline |
| Val | Valine | Boc | tert-Butyloxycarbonyl |
| PABC | p-Aminobenzyl alcohol | Pt/C | Platinum on carbon |
| Pd/C | Palladium on carbon | HPLC | High performance liquid |
| | | | chromatography |
| LCMS | Liquid chromatography -mass | Rf | Retardation factor |
| | spectrometry | | |
| Pre-TLC | Preparative thin layer | DMSO | Dimethyl sulfoxide |
| | chromatography | | |
| PE | Petroleum ether | EA | Ethyl acetate |
| THF | Tetrahydrofuran | DMF | N,N-dimethylformamide |
| MeOH | Methanol | DCM | Dichloromethane |
| IPA | Isopropanol | MeCN | Acetonitrile |
| MTBE | Methyl tert-butyl ether | H₂O | Water |
| PB | Phosphate buffer | EDTA | Ethylenediaminetetraacetic acid |
| Na₂HPO₄ | Disodium hydrogen phosphate | TCEP | Tris(2-carbonylethyl)phosphine |
| | | | hydrochloride |
| DAR | Drug-to-antibody ratio | | |

The structures of the compounds described in the following examples were determined by nuclear magnetic resonance (¹H NMR) or mass spectrometry (MS).

Nuclear magnetic resonance (¹H NMR) was measured using a Bruker 400 MHz nuclear magnetic resonance instrument; the deuterated reagent was hexadeuterated dimethyl sulfoxide (DMSO-d6); and the internal standard substance was tetramethylsilane (TMS).

The abbreviations in the nuclear magnetic resonance (NMR) spectra used in the examples are shown below.
s: singlet, d: doublet, t: triplet, q: quartet, m: multiplet, br: broad, J: coupling constant, Hz: Hertz, DMSO-d6: deuterated dimethyl sulfoxide. δ values are expressed in ppm.

The mass spectrometer (MS) was determined using an Agilent (ESI) mass spectrometer, model: Agilent 6120B.

### Preparation Example 1: Preparation of (R)—N-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)-3-hydroxybutyramide (1-36)

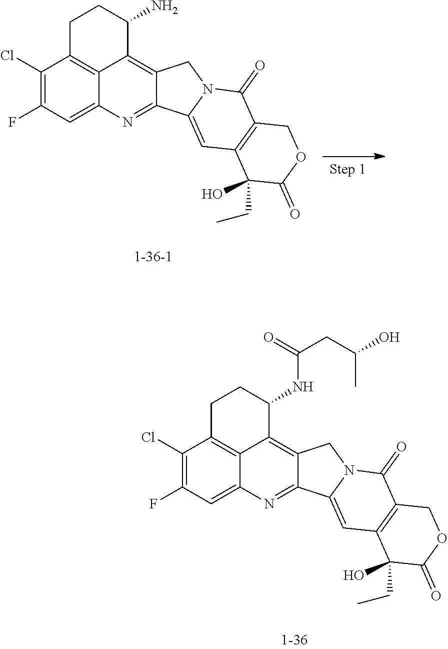 [SMILES: CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3]

(1S,9S)-1-Amino-4-chloro-9-ethyl-5-fluoro-9-hydroxy-1,2,3,9,12,15-hexahydro-10H,13H-benzo[de]pyrano[3′,4′:6,7]indolizino[1,2-b]quinolin-10,13-dione (30 mg, 52.64 μmol), (R)-3-hydroxybutyric acid (10.96 mg, 105.28 μmol), HATU (30.02 mg, 78.96 μmol) and DIPEA (20.41 mg, 157.93 μmol) were dissolved in DMF (2 mL), and the reaction system was stirred at 25° C. for 2 hours. The reaction solution was purified by high performance liquid chromatography and freeze-dried to obtain the title compound (17 mg, 31.42 μmol).

Its separation and purification method was as follows:

Chromatographic column: Waters XBridge Prep
C18OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 20 | 80 | 30 |
| 2.00 | 20 | 80 | 30 |
| 18.00 | 85 | 15 | 30 |

Its structural characterization data are as follows:

ESI-MS (m/z):542.1 [M+H]⁺

¹H NMR (400 MHz, DMSO-d6) δ 8.43 (d, J=8.8 Hz, 1H), 8.06 (d, J=10.4 Hz, 1H), 7.33 (s, 1H), 6.54 (s, 1H), 5.63-5.55 (m, 1H), 5.43 (s, 2H), 5.27 (d, J=18.8 Hz, 1H), 5.21 (d, J=18.8 Hz, 1H), 4.66 (d, J=4.8 Hz, 1H), 4.09-3.98 (m, 1H), 3.32-3.26 (m, 2H), 2.32-2.25 (m, 1H), 2.24-2.12 (m, 3H), 1.93-1.78 (m, 2H), 1.08 (d, J=6.0 Hz, 3H), 0.87 (t, J=7.2 Hz, 3H).

### Preparation Example 2: Preparation of (S)—N-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)-3-hydroxybutyramide (1-39)

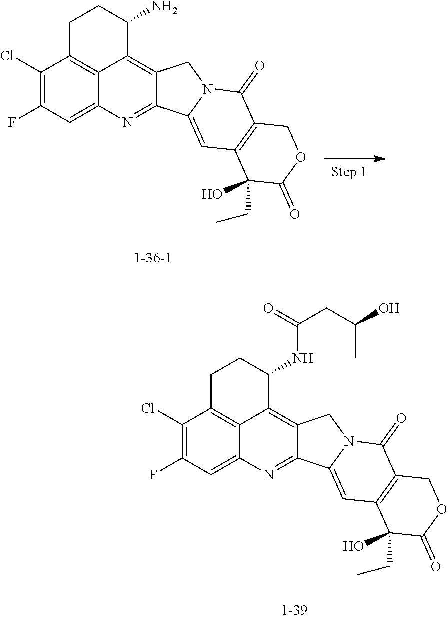 [SMILES: CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3]

(1S,9S)-1-Amino-4-chloro-9-ethyl-5-fluoro-9-hydroxy-1,2,3,9,12,15-hexahydro-10H,13H-benzo[de]pyrano[3′,4′:6,7]indolizino[1,2-b]quinolin-10,13-dione (30 mg, 0.053 mmol, trifluoroacetate) and (S)-3-hydroxybutyric acid (16.5 mg, 0.158 mmol) were dissolved in DMF (0.5 mL), and then added with HATU (40.0 mg, 0.105 mmol) and DIPEA (20.4 mg, 0.158 mmol).

After the addition, the reaction mixture was reacted at room temperature for 2 hours. The reaction solution was directly purified by preparative high performance liquid chromatography and freeze-dried to obtain the title compound (13.5 mg, 0.024 mmol).

Its structural characterization data are as follows:

MS m/z (ESI): 542.1 [M+H]⁺

¹H NMR (400 MHz, DMSO-d6) δ 8.44 (d, J=8.8 Hz, 1H), 8.09 (d, J=10.4 Hz, 1H), 7.34 (s, 1H), 6.56 (m, 1H), 5.64-5.60 (m, 1H), 5.44 (s, 2H), 5.32-5.25 (m, 2H), 4.63 (d, J=4.8 Hz, 1H), 4.06-4.01 (m, 1H), 3.31-3.26 (m, 2H), 2.28-2.23 (m, 1H), 2.20-2.13 (m, 3H), 1.90-1.82 (m, 2H), 1.08 (d, J=6.4 Hz, 3H), 0.87 (t, J=7.2 Hz, 3H).

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

### Intermediate Preparation Example 1: Preparation of 3,6,9,12,15,18,21,24,27,30-decamethyl-36-(2-(methylsulfonyl)pyrimidin-5-yl)-4,7,10,13,16,19,22,25,28,31-decaoxo-3,6,9,12,15,18,21,24,27,30-decaazahexatriacont-35-ynoic acid (INT-1)

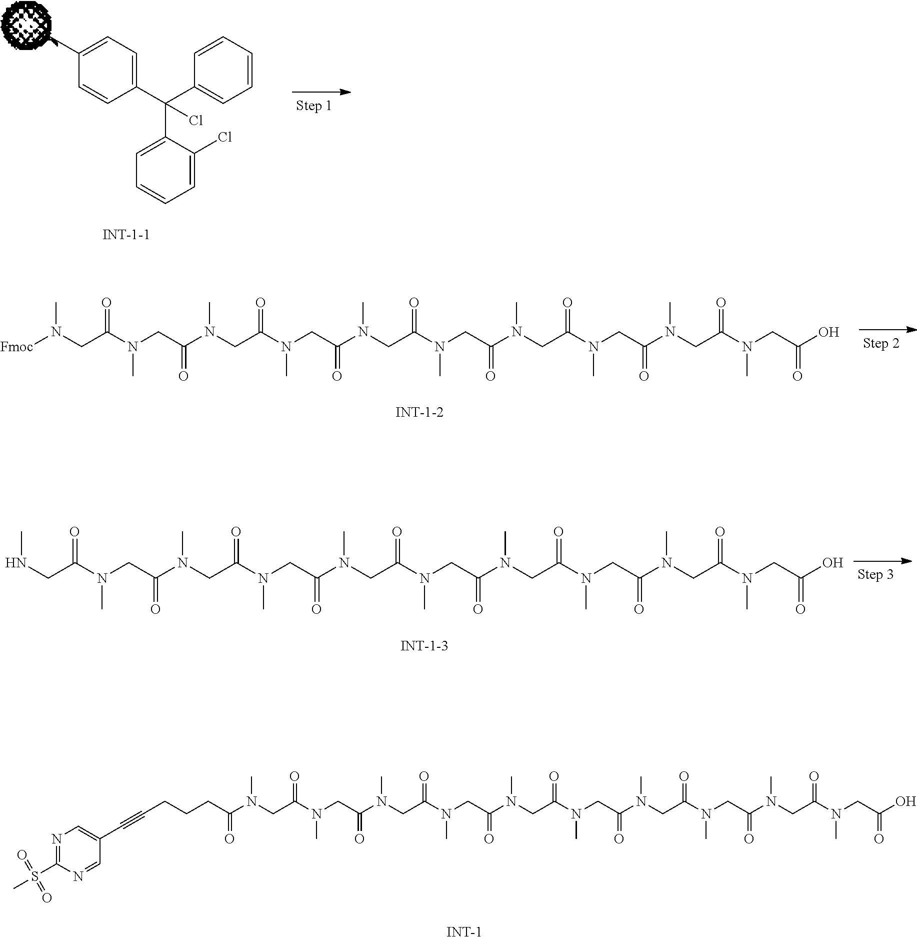

### Step 1: Preparation of 1-(9H-fluoren-9-yl)-4,7,10,13,16,19,22,25,28,31-decamethyl-3,6,9,12,15,18,21,24,27,30-decaoxo-2-oxa-4,7,10,13,16,19,22,25,28,31-decaazatritriacontan-33-oic acid (INT-1-2)

The standard solid-phase synthesis method was used as follows:
1) Preparation of resin: 2-CTC resin (3.00 mmol, 3.70 g, 0.81 mmol/g), Fmoc-sarcosyl-sarcosine (9.00 mol, 3.44 g, 3.00 equivalents) and DIPEA (4.00 equivalents) were added to dichloromethane (10.0 mL), and reacted under nitrogen atmosphere for 2 hours. Then, MeOH (10.0 mL) was added to the resin under nitrogen bubbling atmosphere within 30 minutes, and the resin was obtained by filtration.
2) Coupling: Under nitrogen bubbling, a DMF (10.0 mL) solution of FMOC-sarcosyl-sarcosine (6.88 g, 6.00 equivalents) and HATU (6.58 g, 5.70 equivalents) was added to the resin. After DIPEA (6.00 equivalents) was added dropwise, nitrogen was bubbled at 20° C. for 30 minutes. The resin was washed with DMF (30.0 mL×5) before proceeding to the next step.
3) Deprotection: 20% Piperidine DMF solution (30.0 mL) was added to the resin, and bubbled with nitrogen at 20° C. for 30 minutes. Then, the resin was washed with DMF (30.0 mL)×5.
4) Steps 2 and 3 were repeated using the amino acids shown in Table 1.
5) Then the resin was washed with DMF (30.0 mL×5) and MeOH (30.0 mL×5), and then dried under vacuum.

TABLE 1

| Materials | Coupling Reagent |
|---|---|
| Fmoc-sarcosyl-sarcosine | HATU (5.70 equiv.) and DIPEA (6.00 equiv.) |
| Fmoc-sarcosyl-sarcosine | HATU (5.70 equiv.) and DIPEA (6.00 equiv.) |
| Fmoc-sarcosyl-sarcosine | HATU (5.70 equiv.) and DIPEA (6.00 equiv.) |

Cleavage and purification of peptides:
1) Cleavage solution (TFA/DCM, 1/10, v/v, 200.0 mL) was added to the flask containing the side chain-protected peptide at room temperature, and stirred for 3 minutes twice.
2) After filtration, the filtrates were combined and concentrated.
3) The crude product was purified by preparative high performance liquid chromatography and freeze-dried to obtain the title compound (2856 mg, TFA salt).

Its structural characterization data are as follows:

ESI-MS (m/z): 952.2 (M + H)⁺.
Separation conditions

| Dissolution | DMF | |
|---|---|---|
| conditions | | |
| Instrument | Auno DAC-100 | |
| Mobile phases | A: Water (0.05% TFA in H₂O) | |
| | B: Acetonitrile | |
| Gradient | 20-50% -- 42 min. Retention time: 21 min | |
| Preparative column | Phenomenex Luna C18 250*100 mm, 10 um, | |
| | 100 Å | |
| Flow rate | 250 | mL/min |
| Detection wavelength | 220/254 | nm |
| Temperature | 25° | C. |

Its purification method was as follows:

### Step 2: Preparation of 5,8,11,14,17,20,23,26,29-nonamethyl-4,7,10,13,16,19,22,25,28-nonaoxo-2,5,8,11,14,17,20,23,26,29-decaazahentriacontan-31-oic acid (INT-1-3)

1-(9H-Fluoren-9-yl)-4,7,10,13,16,19,22,25,28,31-decamethyl-3,6,9,12,15,18,21,24,27,30-decaoxo-2-oxa-4,7,10,13,16,19,22,25,28,31-decaazatritriacontan-33-oic acid (200.0 mg, 0.21 mmol) was added into DMF (3 mL), added with diethylamine (46.1 mg, 0.63 mmol), and stirred at room temperature for 1 hour. After the reaction was completed, the solvent was removed by a freeze dryer, 10 mL of ethyl acetate was added, and recrystallization was performed for 1 hour. After filtration, the filter cake was freeze-dried to remove solvent to obtain the title compound (145.4 mg, 0.20 mmol).

Its structural characterization data are as follows:

MS m/z (ESI): 729.2 [M+H]⁺

### Step 3: Preparation of 3,6,9,12,15,18,21,24,27,30-decamethyl-36-(2-(methylsulfonyl) pyrimidin-5-yl)-4,7,10,13,16,19,22,25,28,31-decaoxo-3,6,9,12,15,18,21,24,27,30-decaazahexatriacont-35-ynoic acid (INT-1)

5,8,11,14,17,20,23,26,29-Nonamethyl-4,7,10,13,16,19,22,25,28-nonaoxo-2,5,8,11,14,17,20,23,26,29-decaazahentriacontan-31-oic acid (145.4 mg, 0.20 mmol) was dissolved in DMF (2 mL), added with DIPEA (77.5 mg, 0.6 mmol), and added with A-14-1 (109.6 mg, 0.30 mmol). After the reaction was completed, the title compound (146.2 mg, 0.15 mmol) was obtained by purification by flash column chromatography (C18, water/acetonitrile=0.4) and freeze-drying.

Its structural characterization data are as follows:

MS m/z (ESI): 979.3 [M+H]⁺

### Intermediate Preparation Example 2: Preparation of (6-(2-(methylsulfonyl)pyrimidin-5-yl)hex-5-ynoyl)-L-alanyl-L-alanine (INT-2)

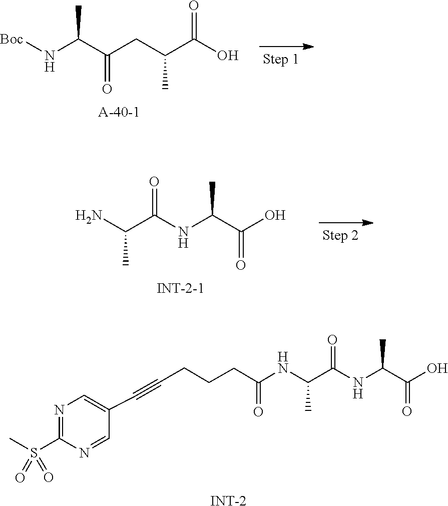 [SMILES: C[C@H](CC(=O)[C@H](C)NC(=O)OC(C)(C)C)C(=O)O ; C[C@H](N)C(=O)N[C@@H](C)C(=O)O ; C[C@H](NC(=O)[C@H](C)NC(=O)CCCC#Cc1cnc(S(C)(=O)=O)nc1)C(=O)O ; "INT-2"]

### Step 1: Preparation of L-alanyl-L-alanine (INT-2-1)

(Tert-butoxycarbonyl)-L-alanyl-L-alanine (100 mg, 384.19 μmol) was dissolved in dichloromethane (2 mL), and added with trifluoroacetic acid (0.5 mL). The reaction was carried out at 20° C. for 1 hour. After the reaction was completed, the reaction solution was concentrated under reduced pressure to remove the solvent to obtain the light yellow title compound (160 mg, 370.93 μmol).

Its structural characterization data are as follows:

MS m/z (ESI): 161.1 [M+H]⁺

### Step 2: Preparation of (6-(2-(methylsulfonyl)pyrimidin-5-yl)hex-5-ynoyl)-L-alanyl-L-alanine (INT-2)

L-Alanyl-L-alanine (160 mg, 412.14 μmol) and 2,5-dioxopyrrolidin-1-yl 6-(2-(methylsulfonyl)pyrimidin-5-yl)hex-5-ynoate (150.58 mg, 412.14 μmol) were dissolved in N,N-dimethylacetamide (2 mL), and added with N,N-diisopropylethylamine (266.33 mg, 2.06 mmol). The reaction was carried out at 20° C. for 1 hour. After the reaction was completed, the title compound (125 mg, 304.55 μmol) was obtained by flash column chromatography (C18, water/acetonitrile=2/1) and freeze-drying.

Its structural characterization data are as follows:

MS m/z (ESI): 428.1 [M+H₂O]⁺

### Intermediate Preparation Example 3: Preparation of (6-(2-(methylsulfonyl)pyrimidin-5-yl)hex-5-ynoyl)glycylglycine (INT-3)

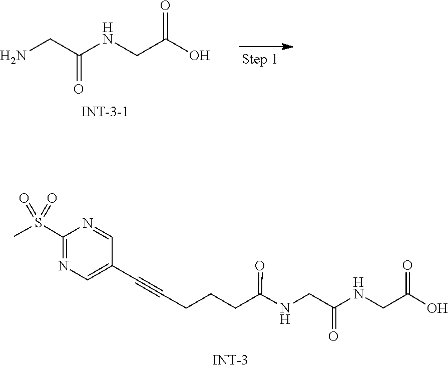 [SMILES: NCC(=O)NCC(=O)O ; CS(=O)(=O)c1ncc(C#CCCCC(=O)NCC(=O)NCC(=O)O)cn1 ; "INT-3"]

Glycylglycine (1 g, 7.57 mmol) and 2,5-dioxo-pyrrolidin-1-yl 6-(2-(methylsulfonyl)pyrimidin-5-yl)hex-5-ynoate (2.77 g, 7.57 mmol) were dissolved in dimethylsulfoxide (10 mL). The reaction was carried out under stirring at room temperature for 2 hours. After the reaction was completed, the title compound (2.52 g, 6.59 mmol) was obtained by flash column chromatography (C18, water/acetonitrile=2/1) and freeze-drying.

Its structural characterization data are as follows:

MS m/z (ESI): 383.2 [M+H]⁺

### Intermediate Preparation Example 4: Preparation of (6-(2-(methylsulfonyl)pyrimidin-5-yl)hex-5-ynoyl)glycylglycyl-L-phenylalanine (INT-4)

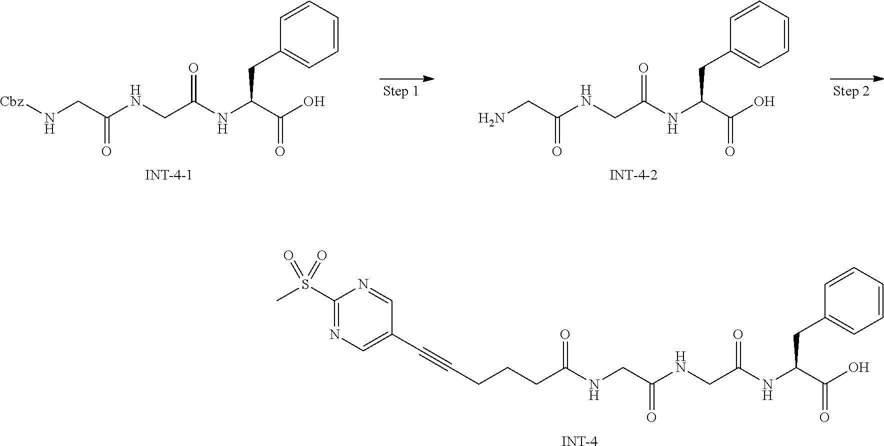 [SMILES: O=C(CNC(=O)OCc1ccccc1)NCC(=O)N[C@@H](Cc1ccccc1)C(=O)O ; NCC(=O)NCC(=O)N[C@@H](Cc1ccccc1)C(=O)O ; CS(=O)(=O)c1ncc(C#CCCCC(=O)NCC(=O)NCC(=O)N[C@@H](Cc2ccccc2)C(=O)O)cn1 ; "INT-4"]

### Step 1: Preparation of glycylglycyl-L-phenylalanine (INT-4-2)

((Benzyloxy)carbonyl)glycylglycyl-L-phenylalanine (200 mg, 483.77 μmol) was dissolved in methanol (8 mL). Palladium-on-carbon (20 mg) was added after nitrogen replacement. The gas was replaced with hydrogen three times, and the reaction was carried out under stirring at room temperature for 3 hours. After the reaction was completed, the palladium-on-carbon was removed by filtration. The filtrate was concentrated to remove the solvent to obtain the title compound (135 mg, 483.37 μmol).

Its structural characterization data are as follows:

MS m/z (ESI): 280.2 [M+H]⁺

### Step 2: Preparation of (6-(2-(methylsulfonyl)pyrimidin-5-yl)hex-5-ynoyl)glycylglycyl-L-phenylalanine (INT-4)

Glycylglycyl-L-phenylalanine (130 mg, 465.46 μmol) and 2,5-dioxopyrrolidin-1-yl 6-(2-(methylsulfonyl)pyrimidin-5-yl)hex-5-ynoate (170.06 mg, 465.46 μmol) were dissolved in N,N-dimethylformamide (8 mL), and added with N,N-diisopropylethylamine (120.32 mg, 930.93 μmol). The reaction was carried out under stirring at room temperature for 1 hour. After the reaction was completed, the title compound (60 mg, 0.11 mmol) was obtained by flash column chromatography (C18, water/acetonitrile=2/1) and freeze-drying.

Its structural characterization data are as follows:

MS m/z (ESI): 530.3 [M+H]⁺

### Intermediate Preparation Example 5: Preparation of (5S,8S,11S)-1-(9H-fluoren-9-yl)-5,8,11-trimethyl-3,6,9,12-tetraoxo-2,15-dioxa-4,7,10,13-tetraazaheptadecan-17-oic acid (INT-5)

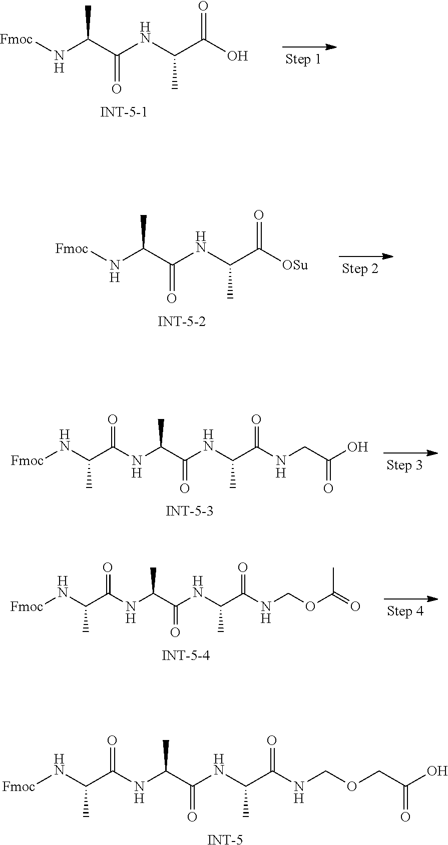

### Step 1: Preparation of 2,5-dioxopyrrolidin-1-yl((9H-fluoren-9-yl)methoxy)carbonyl)-L-alanine-L-alanine (INT-5-2)

(((9H-Fluoren-9-yl)methoxy)carbonyl)-L-alanine-L-alanine (10.0 g, 26.2 mmol) was dissolved in dichloromethane (100 mL), added with DCC (8.09 g, 39.2 mmol, 7.93 mL) and 1-hydroxypyrrolidin-2,5-dione (4.51 g, 39.2 mmol), and stirred at 25° C. for 12 hours. The reaction solution directly filtered (to remove DCU), and the filter cake was rinsed with dichloromethane 3 times (10.0 mL×3). The filtrate was concentrated and added to tetrahydrofuran (100 mL), cooled to 0° C. and continuously stirred for 1 hour. Filtration was performed again (to remove DCU), and the filtrate was concentrated to obtain the title compound as crude product (11.7 g), which was used directly in the next step without purification.

Its structural characterization data are as follows:

ESI-MS (m/z): 502.1 (M+Na)⁺.

### Step 2: Preparation of (((9H-fluoren-9-yl)methoxy)carbonyl)-L-alanine-L-alanine-L-alanine-glycine (INT-5-3)

2,5-Dioxopyrrolidin-1-yl ((9H-fluoren-9-yl)methoxy)carbonyl)-L-alanine-L-alanine (5.00 g, 10.4 mmol) and L-alanine-glycine (1.52 g, 10.4 mmol) were added to a mixed solvent of water (25.0 mL) and tetrahydrofuran (50.0 mL), then added with sodium bicarbonate aqueous solution (1.75 g, 20.8 mmol, 811 μL), and stirred at 25° C. for 2 hours. The reaction solution was adjusted to pH=2 to 3 with 1N dilute hydrochloric acid, extracted with dichloromethane 3 times (60.0 mL×3), the organic phases were combined and dried over anhydrous sodium sulfate, filtered and concentrated to obtain the title compound as crude product (3.18 g, 6.23 mmol), which was used directly in the next step without purification.

Its structural characterization data are as follows:

ESI-MS (m/z): 511.1 (M+H)⁺.

### Step 3: Preparation of (5S,8S,11S)-1-(9H-fluoren-9-yl)-5,8-11-trimethyl-3,6,9,12-tetraoxo-2-oxa-4,7,10,13-tetraazatetradecan-14-yl acetate (INT-5-4)

(((9H-fluoren-9-yl)methoxy)carbonyl)-L-alanine-L-alanine-L-alanine-glycine (3.10 g, 6.07 mmol), copper acetate (882 mg, 4.86 mmol) and lead tetraacetate (6.46 g, 14.5 mmol) were dissolved in DMF (40.0 mL), added with glacial acetic acid (1.60 g, 26.7 mmol, 1.53 mL), then heated to 60° C. and stirred for 1 hour. The reaction solution was directly concentrated to obtain a crude product, which was purified by silica gel column (dichloromethane/methanol=100/1 to 10/1) and then concentrated again to obtain a crude product of the title compound (709 mg, 1.28 mmol), which was used directly in the next step without purification.

Its structural characterization data are as follows:

¹H NMR (400 MHz, DMSO) δ 8.85 (m, 1H), 7.97 (m, 2H), 7.89 (d, J=7.6 Hz, 2H), 7.72 (m, 2H), 7.53 (m, 1H), 7.39-7.44 (m, 2H), 7.30-7.35 (m, 2H), 5.04-5.10 (m, 2H), 4.24-4.28 (m, 3H), 3.17 (d, J=5.2 Hz, 3H), 1.98 (s, 3H), 1.18-1.22 (m, 9H)

### Step 4: Preparation of (5S,8S,11S)-1-(9H-fluoren-9-yl)-5,8,11-trimethyl-3,6,9,12-tetraoxo-2,15-dioxa-4,7,10,13-tetraazaheptadecan-17-oic acid (INT-5)

(5S,8S,11S)-1-(9H-Fluoren-9-yl)-5,8-11-trimethyl-3,6,9,12-tetraoxo-2-oxa-4,7,10,13-tetraazatetradecan-14-yl acetate (300 mg, 571.90 mol) and 2-hydroxyacetic acid (52.2 mg, 686 μmol, 41.7 μL) were dissolved in dichloromethane (3.00 mL), added with PPTS (144 mg, 572 μmol), and then stirred at 25° C. for 2 hours. The reaction solution was added with water (100 mL), adjusted to pH 4-5 with TN dilute hydrochloric acid, and then extracted with dichloromethane 3 times (80.0 mL×3). The organic phases were combined and dried over anhydrous sodium sulfate, then filtered and concentrated to obtain a crude product. The crude product was purified by preparative high-performance liquid chromatography and freeze-dried to obtain the title compound (176 mg, 325.10 μmol).

Its structural characterization data are as follows:

ESI-MS (m/z): 558.2 [M+H]⁺.

Its purification method was as follows:

Chromatographic column: Waters xbridge C18 (150 mm × 25 mm*10 μm)
Mobile phase A: acetonitrile; Mobile phase B: water
(0.05% ammonium bicarbonate)

| Time | Mobile phase A | Mobile phase B | Flow rate |
|---|---|---|---|
| [min] | [%] | [%] | [mL/min] |
| 0.00 | 17 | 83 | 25 |
| 10.00 | 47 | 53 | 25 |

### Intermediate Preparation Example 6: Preparation of 1-(9H-fluoren-9-yl)-3,6,9,12-tetraoxo-2,15-dioxa-4,7,10,13-tetraazaheptadecan-17-oic acid (INT-6)

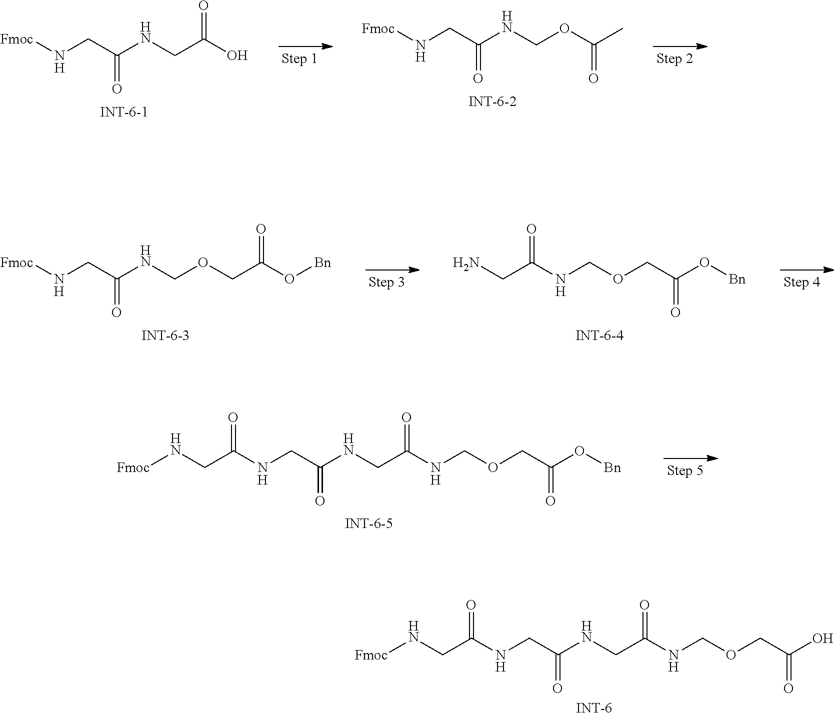

### Step 1: Preparation of methyl (2-((((9H-fluoren-9-yl)methoxy)carbonyl)amino) acetamido)acetate (INT-6-2)

(((9H-Fluoren-9-yl)methoxy)carbonyl)glycylglycine (300 g, 846 mmol) was dissolved in (3 L), added with lead tetraacetate (450 g, 1.02 mol), acetic acid (111 g, 1.86 mol, 106 mL) and copper acetate (61.5 g, 338 mmol), then heated to 60° C. and stirred for 1 hour. The reaction solution was added with water (5 L), and a large amount of solid precipitated, which was filtered and dried to obtain a crude product. The crude product was purified by silica gel column (dichloromethane/methanol=100/1 to 10/1) and concentrated again to obtain the title compound (225 g, 610 mmol).

Its structural characterization data are as follows:

ESI-MS (m/z): 390.9 (M+H)⁺.

### Step 2: Preparation of benzyl 1-(9H-fluoren-9-yl)-3,6-dioxo-2,9-dioxa-4,7-diazaundecan-11-oate (INT-6-3)

Methyl (2-((((9H-fluoren-9-yl)methoxy)carbonyl)amino)acetamido)acetate (225 g, 610 mmol) and benzyl 2-hydroxyacetate (202 g, 1.22 mol, 173 mL) were dissolved in dichloromethane (1.2 L), added with PPTS (23.0 g, 91.6 mmol), then heated to 50° C. and stirred for 12 hours. The reaction solution was added with water (1 L) and extracted with dichloromethane 3 times (500 mL×3). The organic phases were combined and dried over anhydrous sodium sulfate, filtered and concentrated to obtain a crude product, which was purified by silica gel column (dichloromethane/methanol=100/1 to 10/1) and concentrated again to obtain the title compound (215 g, 453 mmol).

Its structural characterization data are as follows:

ESI-MS (m/z): 492.2 (M+H₂O+H)⁺.

### Step 3: Preparation of benzyl 2-((2-aminoacetylamino)methoxy)acetate (INT-6-4)

Benzyl 1-(9H-fluoren-9-yl)-3,6-dioxo-2,9-dioxa-4,7-diazaundecan-11-oate (210 g, 442 mmol) was dissolved in DMF (1.5 L), added with DBU (47.1 g, 309 mmol, 46.70 mL), and stirred at 25° C. for 1 hour. The reaction solution was used directly in the next step without treatment.

Its structural characterization data are as follows:

ESI-MS (m/z): 252.5 (M+H)⁺.

### Step 4: Preparation of benzyl 1-(9H-fluoren-9-yl)-3,6,9,12-tetraoxo-2,15-dioxa-4,7,10,13-tetraazaheptadecan-17-oate (INT-6-5)

To the reaction solution of the previous step, (((9H-Fluoren-9-yl)methoxy) carbonyl)glycylglycine (161 g, 455 mmol), EDCI (96.1 g, 501 mmol) and HOBt (67.7 g, 501 mmol) were added, and then stirred at 25° C. for 1 hour. The reaction system was added with water (2 L), and extracted with ethyl acetate 3 times (1000 mL×3). The organic phases were combined, dried over anhydrous sodium sulfate, filtered and concentrated to obtain a crude product, which was purified by silica gel column (dichloromethane/methanol=100/1 to 10/1) and concentrated again to obtain the title compound (150 g, 254 mmol).

Its structural characterization data are as follows:

ESI-MS (m/z): 558.2 [M+H]⁺.

### Step 5: Preparation of 1-(9H-fluoren-9-yl)-3,6,9,12-tetraoxo-2,15-dioxa-4,7,10,13-tetraazaheptadecan-17-oic acid (INT-6)

Benzyl 1-(9H-fluoren-9-yl)-3,6,9,12-tetraoxo-2,15-dioxa-4,7,10,13-tetraazaheptadecan-17-oate (75 g, 127 mmol) and Pd/C (16 g, 10% purity) were added to a mixed solvent of DMF (200 mL) and tetrahydrofuran (300 mL), and subjected to hydrogenation reaction at 25° C. for 2 hours. The reaction solution was directly filtered, the filter cake was rinsed with DMF 3 times (100 mL×3), and the filtrates were combined and concentrated to obtain a crude product, which was recrystallized with methanol (300 mL) to obtain the title compound (30 g, 52.9 mmol).

Its structural characterization data are as follows:

ESI-MS (m/z): 516.1 [M+H₂O+H]⁺.

### Intermediate Preparation Example 7: Preparation of N²-(6-(2-(methylsulfonyl)pyrimidin-5-yl)hex-5-ynoyl)-N⁶-trityl-L-lysyl-L-alanyl-L-alanine (INT-7)

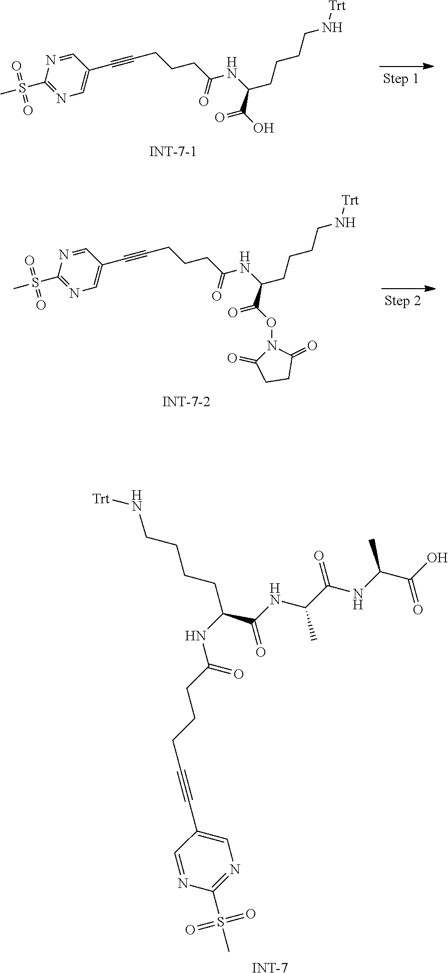

### Step 1: Preparation of 2,5-dioxopyrrolidin-1-yl N-(6-(2-(methylsulfonyl)pyrimidin-5-yl)hex-5-ynoyl)-N⁶-trityl-L-lysinate (INT-7-1)

N²-(6-(2-(Methylsulfonyl)pyrimidin-5-yl)hex-5-ynoyl)-N⁶-trityl-L-lysine (200 mg, 306.37 μmol) and 1-hydroxypyrrolidin-2,5-dione (38.79 mg, 337.01 μmol) were dissolved in DMF (3 mL), added with 1-(3-dimethylaminopropyl)-3-ethylcarbodiimide hydrochloride (70.48 mg, 367.65 μmol), and stirred at room temperature for 3 hours. After the reaction was completed, water was added to the stirred reaction solution, and white solid precipitated. The reaction mixture was continuously stirred for 1 hour and filtered, and the filter cake was dried to obtain the title compound (218 mg, 276.18 μmol).

Its structural characterization data are as follows:

MS m/z (ESI): 750.4 [M+H]⁺

### Step 2: Preparation of N²-(6-(2-(methylsulfonyl)pyrimidin-5-yl)hex-5-ynoyl)-N⁶-trityl-L-lysinyl-L-alanyl-L-alanine (INT-7)

2,5-Dioxopyrrolidin-1-yl N²-(6-(2-(methylsulfonyl)pyrimidin-5-yl)hex-5-ynoyl)-N⁶-trityl-L-lysinate (200 mg, 266.71 μmol) and L-alanyl-L-alanine (42.72 mg, 266.71 μmol) were dissolved in DMF (3 mL), and stirred at room temperature for 2 hours. After the reaction was completed, the reaction solution was purified by reverse phase column chromatography and freeze-dried to obtain the title compound (174 mg, 218.88 μmol).

Its structural characterization data are as follows:

MS m/z (ESI): 795.3 [M+H]⁺

### Example 1: 4-((S)-2-(4-aminobutyl)-35-(4-((6-(2-(methylsulfonyl)pyrimidin-5-yl)hexyl-5-ynamido)methyl)-1H-1,2,3-triazol-1-yl)-4,8-dioxo-6,12,15,18,24,27,30,33-nonaoxy-3,9-diazapentatriacontanamido)benzyl ((1S,9R)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (A-2)

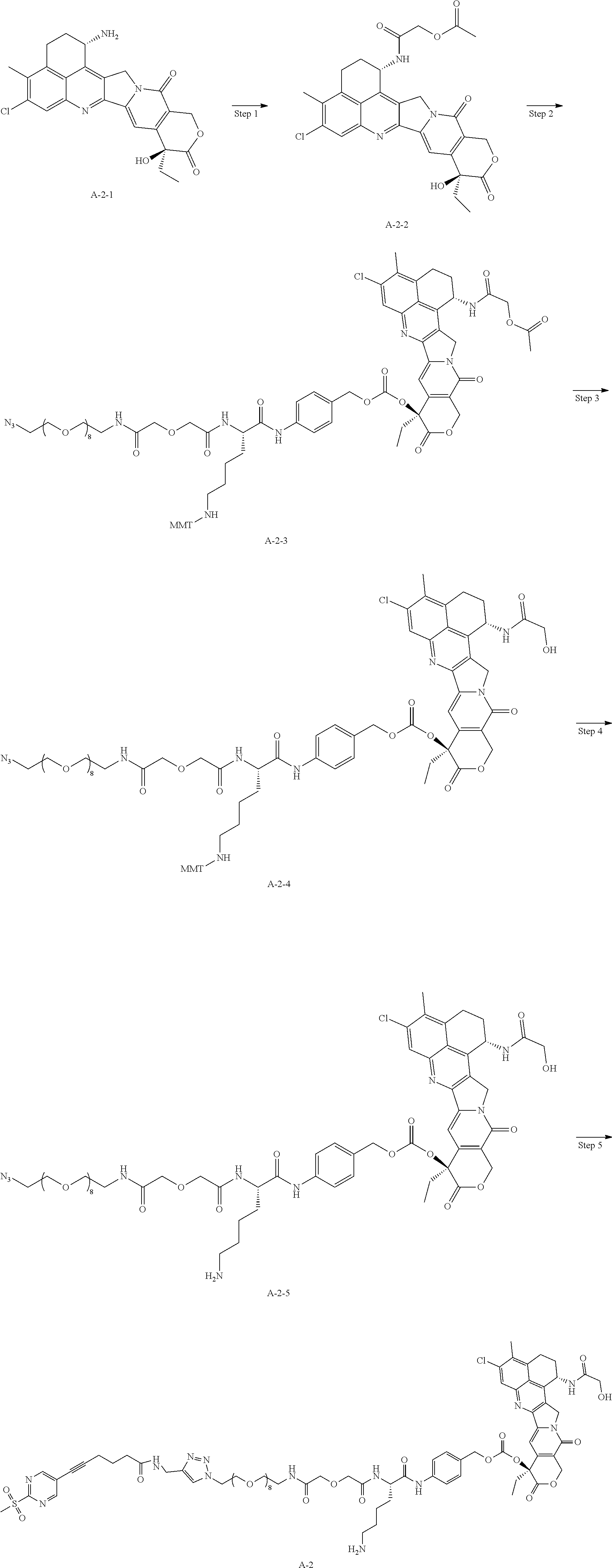

### Step 1: Preparation of ethyl 2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoacetate (A-2-2)

(1S,9S)-1-Amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione (2 g, 3.65 mmol) was dissolved in DMF (50 mL), added dropwise with DIPEA (1.18 g, 9.12 mmol, 1.59 mL), added dropwise with acetoxyacetyl chloride (548.12 mg, 4.01 mmol, 431.59 μL) under ice bath cooling and stirring, then continuously stirred and reacted for 1 hour. The reaction solution was added into 0.1M dilute hydrochloric acid aqueous solution to precipitate solid, and filtered. The filter cake was dissolved in dichloromethane and methanol, dried over anhydrous sodium sulfate, filtered and concentrated to obtain a crude product, which was purified by silica gel column (methanol/dichloromethane=0% to 5%) and concentrated again to obtain the title compound (1.7 g, 3.077 mmol).

Its structural characterization data are as follows:

ESI-MS (m/z): 552.2[M+1]⁺

### Step 2: Preparation of ethyl 2-(((1S,9S)-9-(((4-((S)-35-azido-2-(4-(4-methoxyphenyl) diphenylmethyl)amino)butyl)-4,8-dioxo-6,12,15,18,21,24,27,30,33-nonaoxy-3,9-diazapentatriacontanamido)benzyl)oxy)carbonyl)oxy-5-chloro-9-ethyl-4-methyl-10,13-dioxo-2,3,9,10,13,15-hexahydro-1H,12H-benzo[de]pyrano[3′,4′:6,7]indazolidin[1,2-b]quinolin-1-yl)amino)-2-oxoacetate (A-2-3)

Ethyl 2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoacetate (500 mg, 0.905 mmol) and DMAP (885.33 mg, 7.25 mmol) were dissolved in dry dichloromethane (5 mL), cooled to 0° C. under nitrogen protection, added dropwise with triphosgene (268.81 mg, 0.905 mmol) in dichloromethane (5 mL), kept the temperature, and reacted under stirring for 0.5 hours. (S)-2-(32-azido-5-oxo-3,9,12,15,18,21,24,27,30-nonaoxy-6-azatriamido)-N-(4-(hydroxymethyl)phenyl)-6-(((4-methoxyphenyl)diphenylmethyl)amino)hexanamide (1.44 g, 1.36 mmol) in dichloromethane solution was slowly added dropwise, and the mixture was naturally returned to room temperature and reacted for 4 hours. Water was added to quench the reaction, and the mixture was extracted with dichloromethane 3 times (100 ml×3). The organic phases were combined, washed with saturated brine, dried and concentrated to obtain a crude product, which was purified by silica gel column (MeOH/DCM=0% to 5%) to obtain the title compound (498 mg, 0.304 mmol).

Its structural characterization data are as follows: ESI-MS (m/z): 1352.8[M+1]⁺

### Step 3: Preparation of 4-((S)-35-azido-2-(4-((4-methoxyphenyl)diphenylmethyl)amino) butyl)-4,8-dioxo-6,12,15,18,24,27,30,33-nonaoxy-3,9-diazapentaazatriamido)benzyl ((1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (A-2-4)

Ethyl 2-(((1S,9S)-9-(((4-((S)-35-azido-2-(4-(4-methoxyphenyl)diphenylmethyl)amino) butyl)-4,8-dioxo-6,12,15,18,21,24,27,30,33-nonaoxy-3,9-diazapentabenzotriamido)benzyl)oxy)carbonyl)oxy-5-chloro-9-ethyl-4-methyl-10,13-dioxo-2,3,9,10,13,15-hexahydro-1H,12H-benzo[de]pyrano[3′,4′:6,7]indazolidin[1,2-b]quinolin-1-yl)amino)-2-oxoacetate (200 mg, 0.122 mmol) was dissolved in THF (3 mL) and MeOH (3 mL), added dropwise under stirring with a solution of sodium carbonate (25.88 mg, 0.224 mmol) in water (1 mL). After the addition was completed, the mixture was continuously stirred for 1 hour. Dilute hydrochloric acid was added dropwise into the reaction solution to neutralize the reaction, and the reaction mixture was concentrated under reduced pressure and used directly in the next step.

Its structural characterization data are as follows:

ESI-MS (m/z): 1596.7[M+1]⁺

### Step 4: Preparation of 4-((S)-2-(4-aminobutyl)-35-azido-4,8-dioxo-6,12,15,18,21,24,27,30,33-nonaoxy-3,9-diazapentabenzotriamido)benzyl ((1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (A-2-5)

4-((S)-35-Azido-2-(4-((4-methoxyphenyl)benzhydryl)amino)butyl)-4,8-dioxo-6,12,15,18,24,27,30,33-nonaoxy-3,9-diazapentaazatriamido)benzyl ((1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (190 mg, 119.04 μmol) was dissolved in dichloromethane (5 mL), added with trifluoroacetic acid (0.5 mL), and reacted continuously for 1 hour. Saturated sodium bicarbonate aqueous solution was added dropwise to the reaction solution for neutralization, and then the liquids were separated. The organic phase was concentrated to obtain a crude product, which was purified by reverse phase column chromatography (acetonitrile/1% formic acid aqueous solution=0% to 50%) and freeze-dried to obtain the title compound (95 mg, 69.35 μmol).

Its structural characterization data are as follows:

ESI-MS (m/z):1323.6[M+1]⁺

### Step 5: Preparation of 4-((S)-2-(4-aminobutyl)-35-(4-((6-(2-(methylsulfonyl)pyrimidin-5-yl)hex-5-ynamido)methyl)-1H-1,2,3-triazol-1-yl)-4,8-dioxo-6,12,15,18,24,27,30,33-nonaoxy-3,9-diazapentaazatriamido)benzyl ((1S,9R)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (A-2)

4-((S)-2-(4-Aminobutyl)-35-azido-4,8-dioxo-6,12,15,18,21,24,27,30,33-nonaoxy-3,9-diazapentabenzotriamido)benzyl ((1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (90 mg, 0.066 mmol) and 6-(2-(methylsulfonyl)pyrimidin-5-yl)-N-(prop-2-yn-1-yl)hex-5-ynamide (24.07 mg, 0.079 mmol) were dissolved in DMSO (2 mL) and water (0.2 mL), added with copper bromide (9.42 mg, 0.066 mmol), and stirred continuously for 2 hours. The reaction solution was directly filtered and concentrated to obtain a crude product, which was purified by preparative high performance liquid chromatography and freeze-dried to obtain the title compound (42.2 mg, 24.69 μmol).

Its structural characterization data are as follows:

ESI-MS (m/z): 1628.7[M+1]⁺

Its separation and purification method was as follows:

Chromatographic column: SunFire Prep
C18 OBD 19 mm × 150 mm × 5.0 μm
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 10 | 90 | 28 |
| 2.00 | 10 | 90 | 28 |
| 18.00 | 90 | 10 | 28 |

### Example 2: N—((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxy-5,8,11,14-tetraazahexadecan-16-yl)-6-(2-(methylsulfonyl)pyrimidin-5-yl)hexan-5-amide (A-14)

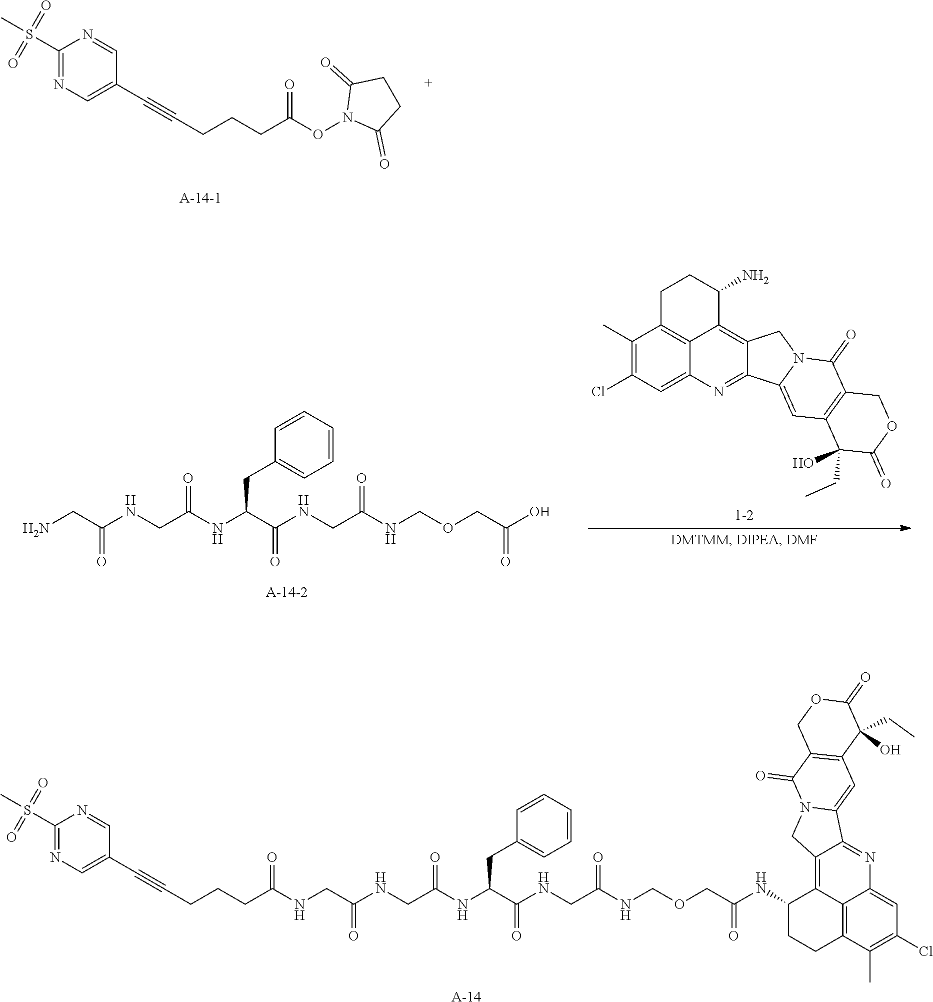 [SMILES: CS(=O)(=O)c1ncc(C#CCCCC(=O)ON2C(=O)CCC2=O)cn1 ; NCC(=O)NCC(=O)N[C@@H](Cc1ccccc1)C(=O)NCC(=O)NCOCC(=O)O ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; "A-14"]

Under nitrogen protection, 2,5-dioxopyrrolidin-1-yl-6-(2-(methylsulfonyl)pyrimidin-5-yl)hexan-5-ynoate (A-14-1, 0.66 g, 1.80 mmol) and (R)-16-amino-10-benzyl-6,9,12,15-tetraoxo-3-oxa-5,8,11,14-tetraazahexadecanoic acid (A-14-2, 0.75 g, 1.77 mmol) were added to DMF (19 mL), heated to 35° C. and reacted for 16 hours. Then the reaction system was added with (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione (1-2, 1.00 g, 1.77 mmol), cooled to 5° C. to 15° C. with ice water, added with DMTMM (0.98 g, 3.53 mmol), then added dropwise with DIPEA (1.14 g, 8.84 mmol), and reacted at 25° C. for 16 hours. The reaction solution was poured into a mixture of DCM (600 mL), IPA (60 mL) and water (100 mL), and stirred for 10 minutes. The DCM phase was separated, and washed with brine (100 mL). The organic phase was concentrated to obtain a crude product, which was purified by preparative high performance liquid chromatography and freeze-dried to obtain 0.98 g of compound A-14.

The separation and purification method of A-14 was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 24 |
| 2.00 | 30 | 70 | 24 |
| 18.00 | 80 | 20 | 24 |

The structural characterization data of A-14 are as follows:

MS m/z (ESI): 1107.3 [M+H]⁺

¹H NMR (400 MHz, DMSO) δ 9.10 (s, 2H), 8.66-8.63 (m, 1H), 8.51 (d, J=8.8 Hz, 1H), 8.34-8.31 (m, 1H), 8.21-8.19 (m, 1H), 8.17-8.09 (m, 2H), 8.08-8.04 (m, 1H), 7.30 (s, 1H), 7.26-7.15 (m, 5H), 6.55 (s, 1H), 5.56-5.55 (m, 1H), 5.48-5.35 (m, 2H), 5.25-5.10 (m, 2H), 4.64 (d, J=6.4 Hz, 2H), 4.45-4.44 (m, 1H), 4.06-3.98 (m, 2H), 3.77-3.52 (m, 6H), 3.41 (s, 3H), 3.25-3.12 (m, 2H), 3.03-3.00 (m, 1H), 2.83-2.72 (m, 1H), 2.58-2.56 (m, 2H), 2.48 (s, 3H), 2.33-2.30 (m, 2H), 2.21-2.13 (m, 2H), 1.91-1.76 (m, 4H), 0.87 (t, J=7.2 Hz, 3H).

### Example 3: N-((7S,10S,13S)-1-(((1S,9S)-5-Chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-7,10-dimethyl-1,6,9,12-tetraoxo-3-oxa-5,8,11-triazatetradecan-13-yl)-6-(2-(methylsulfonyl)pyrimidin-5-yl)hexan-5-amide (A-26)

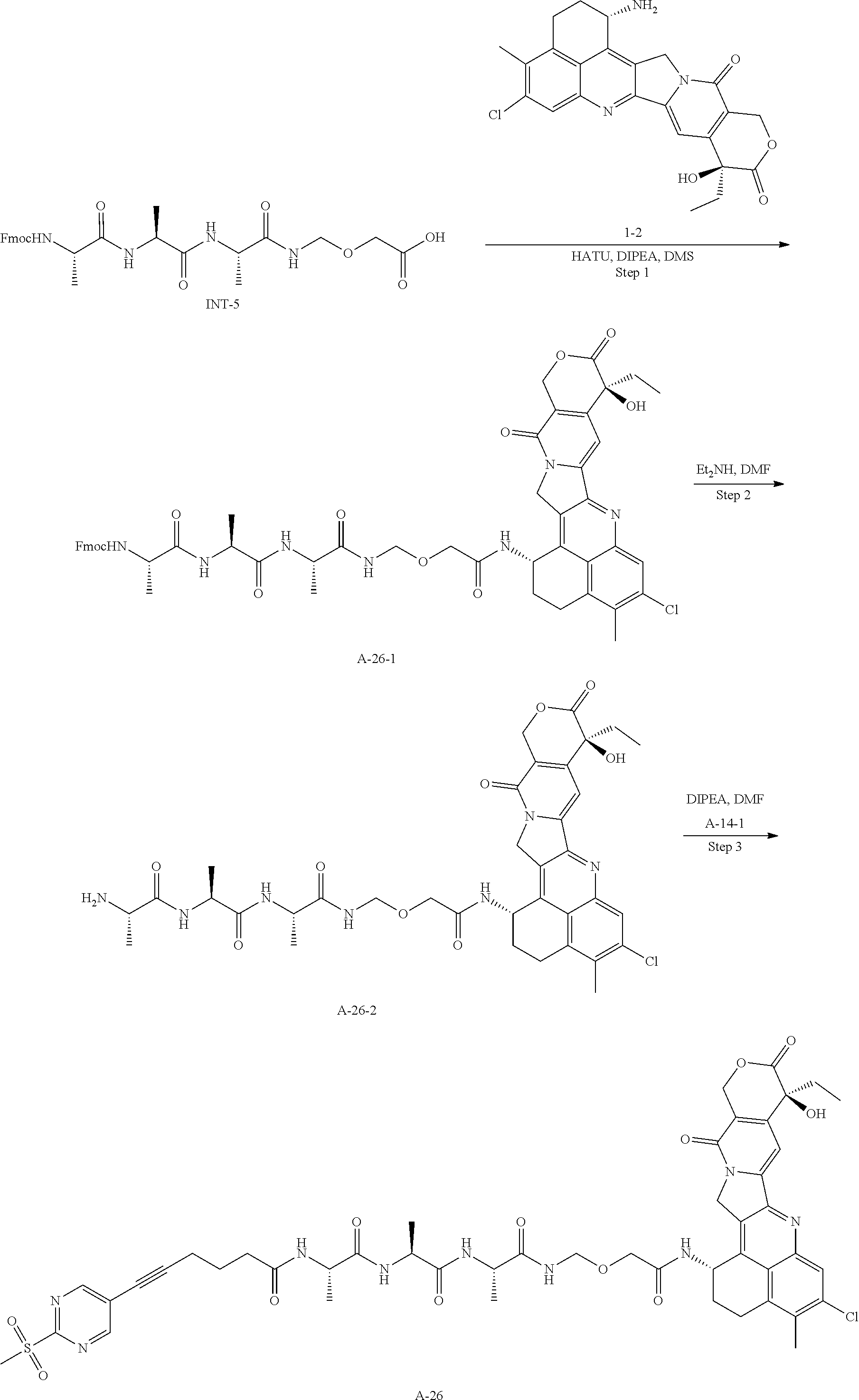

### Step 1

Compound INT-5 (657 mg, 1.22 mmol) and compound 1-2 (500 mg, 1.11 mmol) were dissolved in N,N-dimethylformamide (10 mL), then added with HATU (630.67 mg, 1.66 mmol) and N,N-diisopropylethylamine (428 mg, 3.32 mmol), and stirred at room temperature for 1 hour. After the reaction was completed, the reaction solution was directly purified by high performance liquid chromatography and freeze-dried to obtain 700 mg of compound A-26-1.

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

### Step 2

Compound A-26-1 (500 mg, 0.513 mmol) was dissolved in N,N-dimethylformamide (2 mL), added with diethylamine (75.05 mg, 1.03 mmol), and reacted at room temperature for 1 hour. After the reaction, the reaction solution was directly purified by preparative high performance liquid chromatograph and freeze-dried to obtain 307 mg of compound A-26-2.

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 20 | 80 | 24 |
| 2.00 | 20 | 80 | 24 |
| 18.00 | 80 | 20 | 24 |

### Step 3

A-26-2 (170 mg, 0.226 mmol) and compound A-14-1 (90.83 mg, 0.249 mmol) were dissolved in N,N-dimethylformamide (10 mL), and added with N,N-diisopropylethylamine (29.21 mg, 0.226 mmol). The reaction solution was stirred at room temperature for 16 hours. The reaction solution was directly purified by HPLC and freeze-dried to obtain 50.56 mg of compound A-26.

Its structural characterization data are as follows:

MS m/z (ESI): 1002.4 [M+H]⁺

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

¹H NMR (400 MHz, DMSO) δ 9.11 (s, 2H), 8.68 (t, J=6.4 Hz, 1H), 8.49 (d, J=8.8 Hz, 1H), 8.16 (s, 1H), 8.10 (d, J=7.2 Hz, 1H), 8.01 (d, J=7.2 Hz, 1H), 7.91 (d, J=6.8 Hz, 1H), 7.31 (s, 1H), 6.55 (s, 1H), 5.65-5.55 (m, 1H), 5.43 (s, 2H), 5.21 (s, 2H), 4.67-4.55 (m, 2H), 4.29-4.15 (m, 3H), 3.98 (s, 2H), 3.41 (s, 3H), 3.25-3.15 (m, 2H), 2.57-2.56 (m, 2H), 2.35-2.27 (m, 2H), 2.22-2.12 (m, 2H), 1.91-1.75 (m, 4H), 1.23-1.09 (m, 9H), 0.87 (t, J=7.2 Hz, 3H).

### Example 4: N-((7S,10R,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-7,10-dimethyl-1,6,9,12-tetraoxo-3-oxa-5,8,11-triazatetradecan-13-yl)-6-(2-(methylsulfonyl)pyrimidin-5-yl)hex-5-ynamide (A-38)

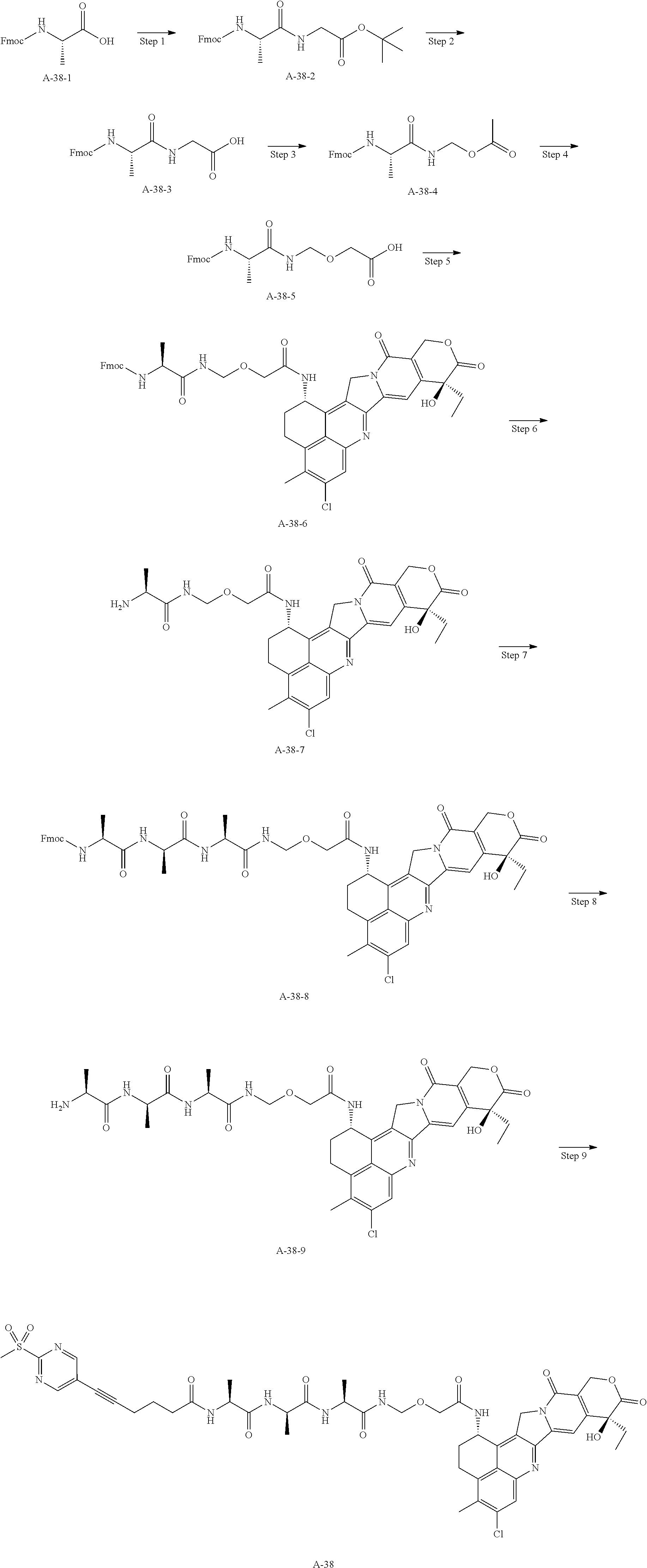

### Step 1: Preparation of tert-butyl ((9H-fluoren-9-yl)methoxy)carbonyl)-L-alanineglycinate (A-38-2)

(((9H-Fluoren-9-yl)methoxy)carbonyl)-L-alanine (350 g, 1.12 mol) and tert-butyl glycinate (147 g, 1.12 mol) were dissolved in dichloromethane (2.5 L), added in sequence with EDCI (237 g, 1.24 mol), DIEA (159 g, 1.24 mol, 215 mL) and HOBt (167 g, 1.24 mol), and stirred at room temperature for 1 hour. The reaction solution was added with water (1 L), and extracted with dichloromethane 3 times (500 mL×3). The organic phases were combined and dried over anhydrous sodium sulfate, filtered and concentrated to obtain a crude product, which was purified by silica gel column (CH₂Cl₂/MeOH=100/1 to 10/1) and concentrated again to obtain the title compound (220 g, 518 mmol).

Its structural characterization data are as follows:

MS m/z (ESI): 442.2 [M+H]⁺

### Step 2: Preparation of (((9H-fluoren-9-yl)methoxy)carbonyl)-L-alanine-glycine (A-38-3)

Tert-butyl (((9H-fluoren-9-yl)methoxy)carbonyl)-L-alanineglycinate (120 g, 282 mmol) was dissolved in dichloromethane (1 L), added with trifluoroacetic acid (32.2 g, 282 mmol, 20.9 mL), and stirred at 25° C. for 2 hours. The reaction solution was concentrated to obtain the title compound as crude product (130 g), which was used directly in the next step without purification.

Its structural characterization data are as follows:

MS m/z (ESI): 369.0 [M+H₂O+H]⁺

### Step 3: Preparation of methyl (S)-(2-((((9H-fluoren-9-yl)methoxy)carbonyl)amino) propionamido)acetate (A-38-4)

(((9H-Fluoren-9-yl)methoxy)carbonyl)-L-alanine-glycine (128 g, 347 mmol) was dissolved in DMF (1 L), added with copper acetate (25.2 g, 138 mmol), lead tetraacetate (184 g, 416.96 mmol) and AcOH (45.9 g, 764 mmol, 43.7 mL), then heated to 60° C. and stirred for 18 hours. The reaction solution was added with water (1.5 L), and extracted with ethyl acetate 3 times (1 L×3). The organic phases were combined and dried over anhydrous sodium sulfate, filtered and concentrated to obtain a crude product, which was purified by silica gel column (CH₂Cl₂/MeOH=100/1 to 10/1) and concentrated again to obtain the title compound (70 g, 183.05 mmol).

Its structural characterization data are as follows:

MS m/z (ESI): 400.0 [M+H₂O+H]⁺

### Step 4: Preparation of (S)-1-(9H-fluoren-9-yl)-5-methyl-3,6-dioxo-2,9-dioxa-4,7-diazaundecan-11-oic acid (A-38-5)

Methyl (S)-(2-((((9H-fluoren-9-yl)methoxy)carbonyl)amino)propionamido)acetate (2.7 g, 7.06 mmol) and glycolic acid (1.61 g, 21.18 mmol) were dissolved in THF (50 mL), added with p-toluenesulfonic acid (36.78 mg, 706.05 μmol), and reacted at 20° C. for 4 hours. The organic phase was diluted with water, adjusted to pH 6 with 1N dilute hydrochloric acid, and extracted with ethyl acetate 3 times (50 mL×3), the organic phases were combined and dried over anhydrous sodium sulfate, filtered and concentrated to obtain a crude product. The crude product was purified by silica gel column chromatography (MeOH/DCM=5 to 10%) and concentrated again to obtain the title compound (1.83 g, 4.14 mmol).

Its structural characterization data are as follows:

MS m/z (ESI): 399.1 [M+H]⁺

### Step 5: Preparation of (9H-fluoren-9-yl)methyl ((S)-1-(((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)carbamate (A-38-6)

(S)-1-(9H-Fluoren-9-yl)-5-methyl-3,6-dioxo-2,9-dioxa-4,7-diazaundecan-11-oic acid (200 mg, 0.50 mmol) and (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (275 mg, 0.50 mmol) were dissolved in DMF (10 mL), then added with DMTMM (295.91 mg, 1.00 mmol) and DIPEA (259.52 mg, 2.01 mmol), and stirred at room temperature for 2 hours. The reaction solution was directly purified by reverse phase column chromatography (acetonitrile/1% formic acid aqueous solution=0% to 70%) and freeze-dried to obtain the title compound (220 mg, 264.33 μmol).

Its structural characterization data are as follows:

MS m/z (ESI): 832.3 [M+H]⁺

### Step 6: Preparation of (S)-2-amino-N-((2-(((1S,9S)-5-chloro-9-ethyl-9-hydroxy-4-methyl-10,13-dioxo-2,3,9,13,15-hexahydro-1H,12H-benzo[de]pyrano[3′,4′:6,7]indolizino[1,2-b]quinolin-1-yl)amino)-2-oxoethoxy)methyl)propanamide (A-38-7)

(9H-Fluoren-9-yl)methyl ((S)-1-(((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)carbamate (220 mg, 0.26 mmol) was dissolved in N,N-dimethylformamide (2 mL), added with diethylamine (38.66 mg, 0.53 mmol), and reacted at room temperature for 1 hour. The reaction solution was directly purified by reverse phase column chromatography (acetonitrile/1% formic acid aqueous solution=0% to 50%) and freeze-dried to obtain the title compound (120 mg, 196.70 μmol).

Its structural characterization data are as follows:

MS m/z (ESI): 610.4 [M+H]⁺

### Step 7: Preparation of (9H-fluoren-9-yl)methyl ((7S,10R,13S)-1-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-7,10-dimethyl-1,6,9,12-tetraoxo-3-oxa-5,8,11-triazatetradecan-13-yl)carbamate (A-38-8)

(S)-2-Amino-N-((2-(((1S,9S)-5-chloro-9-ethyl-9-hydroxy-4-methyl-10,13-dioxo-2,3,9,13,15-hexahydro-1H,12H-benzo[de]pyrano[3′,4′:6,7]indolizino[1,2-b]quinolin-1-yl)amino)-2-oxoethoxy)methyl)propanamide (120 mg, 0.20 mmol) and (((9H-fluoren-9-yl)methoxy)carbonyl)-L-alanine-D-alanine (97.79 mg, 0.26 mmol) were dissolved in DMF (10 mL), then added with DMTMM (115.95 mg, 0.39 mmol) and DIPEA (101.69 mg, 0.79 mmol). After nitrogen replacement three times, the reaction solution was stirred at room temperature for 2 hours. The reaction solution was directly purified by reverse phase column chromatography (acetonitrile/1% formic acid aqueous solution=0% to 60%) and freeze-dried to obtain the title compound (18 mg, 18.47 μmol).

Its structural characterization data are as follows:

MS m/z (ESI): 974.4 [M+H]⁺

### Step 8: Preparation of (S)-2-amino-N—((R)-1-(((S)-1-(2-((((1S,9S)-5-chloro-9-ethyl-9-hydroxy-4-methyl-10,13-dioxo-2,3,9,13,15-hexahydro-1H,12H-benzo[de]pyrano[3′,4′:6,7]indolizino[1,2-b]quinolin-1-yl)amino)-2-oxoethoxy)methyl)amino)-1-oxopropyl-2-yl)amino (A-38-9)

(9H-Fluoren-9-yl)methyl ((7S,10R,13S)-1-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-7,10-dimethyl-1,6,9,12-tetraoxo-3-oxa-5,8,11-triazatetradecan-13-yl)carbamate (18 mg, 0.018 mmol) was dissolved in DMF (10 mL), added with diethylamine (2.70 mg, 0.037 mmol), subjected to nitrogen replacement three times, and stirred at room temperature for 1 hour. The reaction solution was directly dried under reduced pressure to remove solvent to obtain 18 mg of the crude product of the title compound.

Its structural characterization data are as follows:

MS m/z (ESI): 752.4 [M+H]⁺

### Step 9: Preparation of N-((7S,10R,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-7,10-dimethyl-1,6,9,12-tetraoxo-3-oxa-5,8,11-triazatetradecan-13-yl)-6-(2-(methylsulfonyl)pyrimidin-5-yl)hex-5-ynamide (A-38)

(S)-2-Amino-N—((R)-1-(((S)-1-(2-((((1S,9S)-5-chloro-9-ethyl-9-hydroxy-4-methyl-10,13-dioxo-2,3,9,13,15-hexahydro-1H,12H-benzo[de]pyrano[3′,4′:6,7]indolizino[1,2-b]quinolin-1-yl)amino)-2-oxoethoxy)methyl)amino)-1-oxopropyl-2-yl)amino (18 mg, 0.024 mmol) and 2,5-dioxopyrrolidin-1-yl 6-(2-methylsulfonylpyrimidin-5-yl)hex-5-ynoate (8.74 mg, 0.024 mmol) were dissolved in DMF (5 mL), and DIPEA (9.28 mg, 0.072 mmol) was added to the reaction solution. After nitrogen replacement three times, the reaction solution was stirred at room temperature for 16 hours. After the reaction was completed, the reaction solution was directly purified by preparative high performance liquid chromatography and freeze-dried to obtain the title compound (10.64 mg, 10.61 μmol).

Its structural characterization data are as follows:

MS m/z (ESI): 1002.3 [M+H]⁺

Its separation and purification method was as follows:

Chromatographic column: SHIMADZU Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 20 | 80 | 28 |
| 5.00 | 20 | 80 | 28 |
| 24.00 | 90 | 10 | 28 |

### Example 5: N—(((S)-1-(((S)-1-(2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)amino)-1-oxopropan-2-yl)-6-(2-(methylsulfonyl)pyrimidin-5-yl)hex-5-ynamide (A-40)

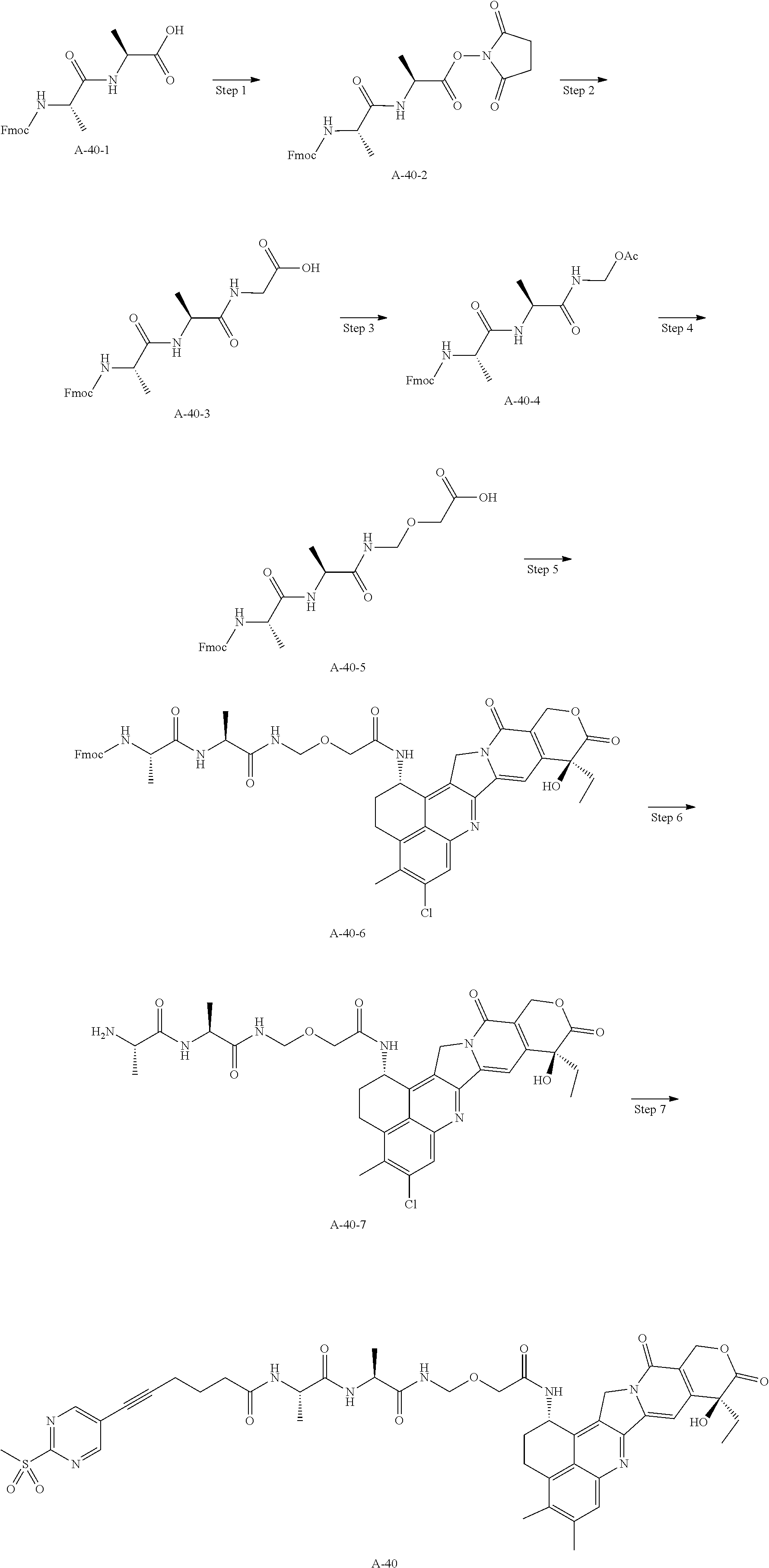

### Step 1: Preparation of 2,5-dioxopyrrolidin-1-yl ((9H-fluoren-9-yl)methoxy)carbonyl)-L-alanine-L-alanine (A-40-2)

(((9H-Fluoren-9-yl)methoxy)carbonyl)-L-alanine-L-alanine (2 g, 5.23 mmol) was dissolved in THF (20 mL), added with HOSu (722.29 mg, 6.28 mmol) and DCC (1.19 g, 5.75 mmol), and stirred at room temperature for 3 hours. Solids gradually precipitated from the system. After the reaction was completed, the reaction solution was directly filtered, the filter cake was washed with an appropriate amount of THF, and the filtrate was dried to obtain 3.4 g of the title compound as crude product, which was directly used in the next step without purification.

Its structural characterization data are as follows:

MS m/z (ESI): 497.2 [M+H+H₂O]⁺

### Step 2: Preparation of (((9H-fluoren-9-yl)methoxy)carbonyl)-L-alanine-L-alanine-glycine (A-40-3)

2,5-Dioxopyrrolidin-1-yl ((9H-fluoren-9-yl)methoxy)carbonyl)-L-alanine-L-alanine (1.8 g, 3.75 mmol) and glycine (563.61 mg, 7.51 mmol) were dissolved in acetone (12 mL) and water (6 mL), then added with NaHCO₃ (630.73 mg, 7.51 mmol), and stirred at room temperature for 2 hours. The reaction solution was added with water (20 mL), and adjusted to pH 3-4 with concentrated hydrochloric acid, and extracted with ethyl acetate 3 times (15 mL×3). The organic phases were combined and washed with saturated sodium chloride aqueous solution (15 mL), dried over anhydrous sodium sulfate, filtered and concentrated to obtain 1.2 g of a crude product of the title compound, which was used directly in the next step without purification.

Its structural characterization data are as follows:

MS m/z (ESI): 440.2 [M+H]⁺

### Step 3: Preparation of (5S,8S)-1-(9H-fluoren-9-yl)-5,8-dimethyl-3,6,9-trioxo-2-oxa-4,7,10-triazaundecan-11-yl acetate (A-40-4)

(((9H-Fluoren-9-yl)methoxy)carbonyl)-L-alanine-L-alanine-glycine (1.2 g, 2.73 mmol) was dissolved in THF (20 mL), and added with pyridine (647.97 mg, 8.19 mmol) and lead tetraacetate (3.63 g, 8.19 mmol). The system turned yellow. After nitrogen replacement, the system was heated to 70° C. for 4 hours. The reaction solution was added with water (20 mL), and extracted with ethyl acetate 3 times (10 mL×3). The organic phases were combined and washed with saturated aqueous ammonium chloride solution (10 mL) and saturated aqueous sodium chloride solution (10 mL) in turn, dried over anhydrous sodium sulfate, filtered and concentrated, and the residue was purified by silica gel column chromatography (ethyl acetate/petroleum ether=50% to 100%) and concentrated again to obtain the title compound (346 mg, 762.97 μmol).

Its structural characterization data are as follows:

MS m/z (ESI): 471.2 [M+H₂O+H]⁺

### Step 4: Preparation of (5S,8S)-1-(9H-fluoren-9-yl)-5,8-dimethyl-3,6,9-trioxo-2,12-dioxa-4,7,10-triazatetradecan-14-oic acid (A-40-5)

(5S,8S)-1-(9H-Fluoren-9-yl)-5,8-dimethyl-3,6,9-trioxo-2-oxa-4,7,10-triazaundecan-11-yl acetate (80 mg, 176.41 μmol) was dissolved in THF (2 mL), added with glycolic acid (67.08 mg, 882.05 μmol), then added with TsOH (3.04 mg, 17.64 μmol), and stirred at room temperature for 3 hours. After the reaction was completed, the reaction solution was spin-dried under reduced pressure to remove the solvent, and the residue was purified by preparative high performance liquid chromatography and freeze-dried to obtain 45 mg of the title compound.

Its structural characterization data are as follows:

MS m/z (ESI): 492.1 [M+Na]⁺

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 20.00 | 70 | 30 | 28 |

### Step 5: Preparation of (9H-fluoren-9-yl)methyl ((S)-1-((S)-1-(2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)amino (A-40-6)

(5S,8S)-1-(9H-Fluoren-9-yl)-5,8-dimethyl-3,6,9-trioxo-2,12-dioxa-4,7,10-triazatetradecan-14-oic acid (25 mg, 42.60 μmol) and (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (23.34 mg, 42.60 μmol) were dissolved in DMF (1.5 mL), added with DIPEA (11.01 mg, 85.20 μmol) and HATU (19.44 mg, 51.12 μmol), and stirred at room temperature for 0.5 hours. After the reaction was completed, the reaction solution was directly used in the next reaction without treatment.

Its structural characterization data are as follows:

MS m/z (ESI): 903.3[M+H]⁺

### Step 6: Preparation of (S)-2-amino-N—((S)-1-((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)propanamide (A-40-7)

DMF (1 mL) and diethylamine (0.2 mL) were added to the reaction solution containing (9H-fluoren-9-yl)methyl ((S)-1-((S)-1-(2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropyl-2-yl)amino (38 mg, 42.06 μmol) in the previous step, and stirred at room temperature for 1 hour. After the reaction was completed, the reaction solution was directly evaporated under reduced pressure to remove diethylamine, and the residue was directly used in the next step.

Its structural characterization data are as follows:

MS m/z (ESI): 699.3 [M+H₂O+H]⁺

### Step 7: Preparation of N—(((S)-1-(((S)-1-(2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)amino)-1-oxopropan-2-yl)-6-(2-(methylsulfonyl)pyrimidin-5-yl)hex-5-ynamide (A-40)

(S)-2-Amino-N—((S)-1-((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)propanamide (28 mg, 41.11 μmol) was dissolved in DMF (1 mL), added with DIPEA (10.63 mg, 82.22 μmol), then added with 2,5-dioxopyrrolidin-1-yl 6-(2-(methylsulfonyl)pyrimidin-5-yl)hex-5-ynoate (15.02 mg, 41.11 μmol), and stirred at room temperature for 1 hour. The reaction solution was directly purified by preparative high performance liquid chromatography and freeze-dried to obtain 45 mg of the title compound.

Its structural characterization data are as follows:

MS m/z (ESI): 931.3 [M+H]⁺

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 25 | 75 | 28 |
| 2.00 | 25 | 75 | 28 |
| 20.00 | 90 | 10 | 28 |

### Example 6: N-(1-(1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12-tetraoxo-3-oxa-5,8,11-triazatridecan-13-yl)-6-(2-(methylsulfonyl)pyrimidin-5-yl)hex-5-ynamide (A-42)

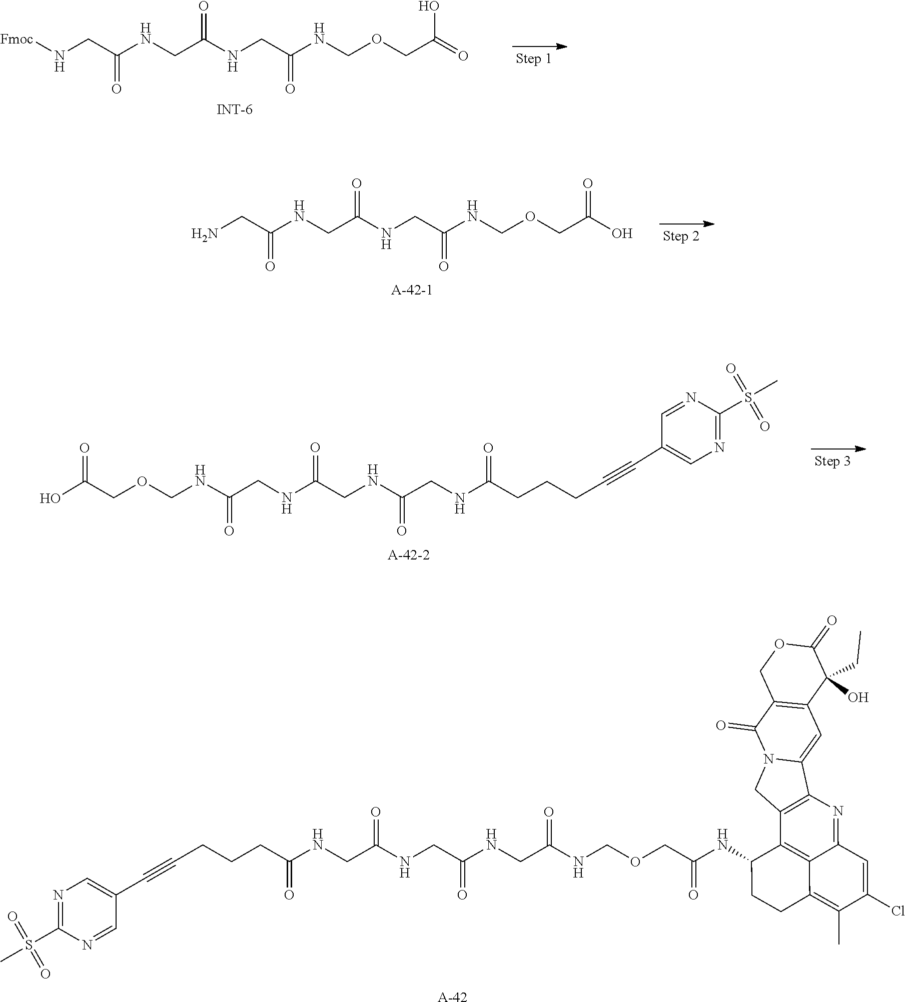

### Step 1: Preparation of 13-amino-6,9,12-trioxo-3-oxa-5,8,11-triazatridecanoic acid (A-42-1)

1-(9H-Fluoren-9-yl)-3,6,9,12-tetraoxo-2,15-dioxa-4,7,10,13-tetraazaheptadecan-17-oic acid (50 mg, 0.1 mol) and diethylamine (0.5 mL) were dissolved in DMF (2.5 mL), and stirred at room temperature for 1 hour. Diethylamine was removed by spin-drying, and the crude product was used directly in the next step.

### Step 2: Preparation of 20-(2-methylsulfonyl)pyrimidin-5-yl)-6,9,12,15-tetraoxo-3-oxa-5,8,11,14-tetraaza-19-ynoic acid (A-42-2)

2,5-Dioxopyrrolidin-1-yl 6-(2-(methylsulfonyl)pyrimidin-5-yl)hex-5-ynoate (71.42 mg, 195.48 μmol) was added to the system in the previous step, and stirred at 25° C. for 1 hour. Methyl tert-butyl ether (30 mL) was added to the reaction solution, and solid precipitated. The crude product (50 mg) was obtained by filtration, and used directly in the next step without purification.

### Step 3: Preparation of N-(1-(1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12-tetraoxo-3-oxa-5,8,11-triazatridecan-13-yl)-6-(2-(methylsulfonyl)pyrimidin-5-yl)hex-5-ynamide (A-42)

20-(2-Methylsulfonyl)pyrimidin-5-yl)-6,9,12,15-tetraoxo-3-oxa-5,8,11,14-tetraaza-19-ynoic acid (50 mg) and (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione (30 mg, 56.38 μmol) were dissolved in DMF (1 mL), then added with HATU (21.65 mg, 56.98 μmol) and N,N-diisopropylethylamine (21.23 mg, 164.23 μmol), and stirred at room temperature for 1 hour. After the reaction was completed, the reaction solution was directly purified by preparative high performance liquid chromatography and freeze-dried to obtain the title compound (11 mg, 11.36 μmol).

Its structural characterization data are as follows:

MS m/z (ESI): 961.3 [M+H]⁺

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

### Example 7: N—(R)-1-((R)-1-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-ylamino)-3-methyl-1-oxobutan-2-yl)-6-(2-(methylsulfonyl)pyrimidin-5-yl)hexan-5-carboxamide (A-44)

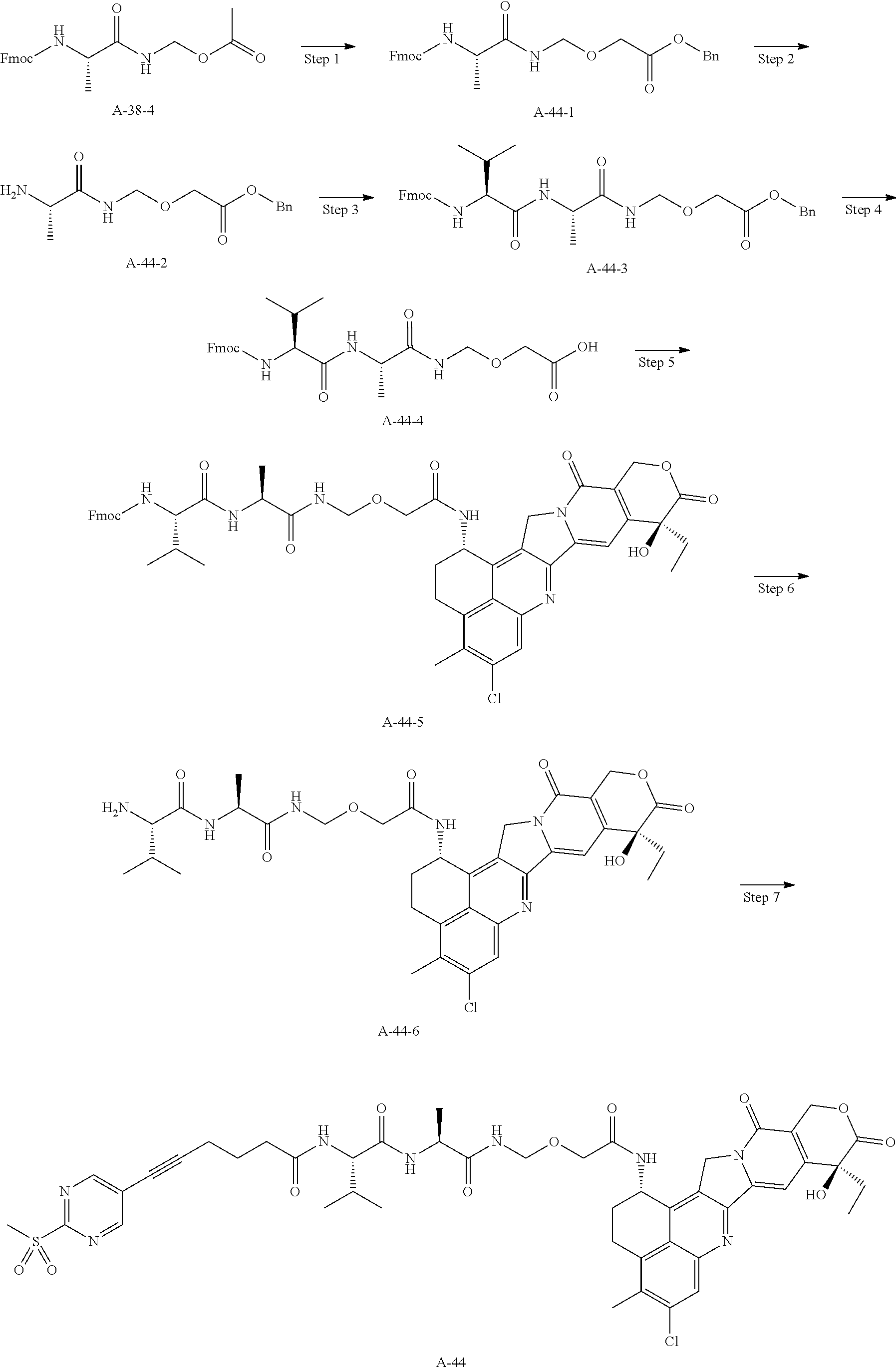

### Step 1: Preparation of benzyl (S)-1-(9H-fluoren-9-yl)-5-methyl-3,6-dioxo-2,9-dioxa-4,7-diazaundecan-11-oate (A-44-1)

Methyl (S)-(2-((((9H-fluoren-9-yl)methoxy)carbonyl)amino)propionamido)acetate (60 g, 156 mmol) and benzyl 2-hydroxyacetate (52.1 g, 313 mmol, 44.5 mL) were dissolved in dichloromethane (500 mL), added with PPTS (19.71 g, 78.45 mmol), then heated to 50° C. and stirred for 24 hours. The reaction solution was added with water (500 mL), and extracted with dichloromethane 3 times (500 mL×3). The organic phases were combined and dried over anhydrous sodium sulfate, filtered, and concentrated to obtain a crude product, which was purified by silica gel column (CH₂Cl₂/MeOH=100/1 to 10/1), then concentrated again to obtain the title compound (45 g, 92.11 mmol).

Its structural characterization data are as follows:

MS m/z (ESI): 506.2 [M+H₂O+H]⁺

### Step 2: Preparation of benzyl (S)-2-((2-aminopropionamido)methoxy)acetate (A-44-2)

Benzyl (S)-1-(9H-fluoren-9-yl)-5-methyl-3,6-dioxo-2,9-dioxa-4,7-diazaundecan-11-oate (40 g, 81.88 mmol) was dissolved in DMF (500 mL), added with DBU (9.97 g, 65.50 mmol, 9.87 mL), and then stirred at 25° C. for 1 hour. The reaction solution was used directly in the next step without treatment.

Its structural characterization data are as follows:

MS m/z (ESI): 267.1 [M+H]⁺

### Step 3: Preparation of benzyl (5S,8S)-1-(9H-fluoren-9-yl)-5-isopropyl-8-methyl-3,6,9-trioxo-2,12-dioxa-4,7,10-triazatetradecan-14-oate (A-44-3)

Benzyl (S)-2-((2-aminopropionamido)methoxy)acetate (2.18 g, 8.19 mmol) and (((9H-fluoren-9-yl)methoxy)carbonyl)-L-valine (2.78 g, 8.19 mmol) were dissolved in DMF (20 mL), added with EDCI (1.88 g, 9.82 mmol) and HOBt (1.33 g, 9.82 mmol) in sequence, and stirred at 25° C. for 1 hour. The reaction solution was added with water (300 mL), and a large amount of solid precipitated, which was filtered to obtain a crude product. The crude product was slurried (added to a mixed solvent of petroleum ether/ethyl acetate=3/1 (50 mL×2), and stirred at 25° C. for 8 hours) to obtain the title compound (4.50 g, 7.66 mmol).

Its structural characterization data are as follows:

MS m/z (ESI): 610.4 [M+Na]⁺

### Step 4: Preparation of (5S,8S)-1-(9H-fluoren-9-yl)-5-isopropyl-8-methyl-3,6,9-trioxo-2,12-dioxa-4,7,10-triazatetradecan-14-oic acid (A-44-4)

Benzyl (5S,8S)-1-(9H-fluoren-9-yl)-5-isopropyl-8-methyl-3,6,9-trioxo-2,12-dioxa-4,7,10-triazatetradecan-14-oate (2 g, 3.40 mmol) and 10% Pd/C (500 mg, 3.40 mmol) were added to DMF (20 mL), subjected to hydrogen replacement 3 times, and then hydrogenation reaction for 5 hours. The reaction solution was filtered to obtain a crude product, which was slurried (added to a mixed solvent of petroleum ether/ethyl acetate=3/1 (30 mL×2) and stirred at 25° C. for 8 hours) to obtain the title compound (1.45 g, 2.60 mmol).

Its structural characterization data are as follows:

MS m/z (ESI): 515.1 [M+H₂O+H]⁺

### Step 5: Preparation of (9H-fluoren-9-yl)methyl ((7S,10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-7,10-dimethyl-1,6,9,12-tetraoxo-3-oxa-5,8,11-triazatetradecan-13-yl)carbamate (A-44-5)

(1S,9S)-1-Amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione (30 mg, 54.74 μmol) and (5S,8S)-1-(9H-fluoren-9-yl)-5-isopropyl-8-methyl-3,6,9-trioxo-2,12-dioxa-4,7,10-triazatetradecan-14-oic acid (29.96 mg, 60.22 μmol) were dissolved in N,N-dimethylformamide (1 mL), then added with HATU (24.96 mg, 65.69 μmol) and N,N-diisopropylethylamine (21.23 mg, 164.23 μmol), and stirred at room temperature for 1 hour. After the reaction was completed, the reaction solution was directly purified by preparative high performance liquid chromatography and freeze-dried to obtain the title compound (50 mg, 53.68 μmol).

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 20 | 80 | 28 |
| 2.00 | 20 | 80 | 28 |
| 18.00 | 80 | 20 | 28 |

### Step 6: Preparation of (S)-2-amino-N—((S)-1-(((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)-3-methylbutanamide (A-44-6)

(9H-Fluoren-9-yl)methyl ((7S,05S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-7,10-dimethyl-1,6,9,12-tetraoxo-3-oxa-5,8,11-triazatetradecan-13-yl)carbamate (50 mg, 53.68 μmol) was dissolved in N,N-dimethylformamide (2 mL), added with diethylamine (1 mL, 53.68 μmol), and reacted at room temperature for 1 hour. After the reaction, the reaction solution was initially concentrated to obtain a DMF solution of the crude product of the title compound, which was used directly in the next step without purification.

Step 7: Preparation of N—(R)-1-((R)-1-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropyl-2-ylamino)-3-methyl-1-oxobutan-2-yl)-6-(2-(methylsulfonyl)pyrimidin-5-yl)hex-5-formamide (A-44)

(S)-2-Amino-N—((S)-1-(((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)-3-methylbutanamide (38 mg, 53.58 μmol) was dissolved in DMF (1 mL), added with N,N-diisopropylethylamine (20.78 mg, 160.75 μmol) and 2,5-dioxopyrrolidin-1-yl 6-(2-(methylsulfonyl)pyrimidin-5-yl)hex-5-ynoate (39.15 mg, 107.16 μmol) in sequence, and dissolved in the concentrated solution of the previous step. The reaction solution was stirred at room temperature for 1 hour. After the reaction, the reaction solution was directly purified by preparative high performance liquid chromatography and freeze-dried to obtain the title compound (5 mg, 5.11 μmol).

Its structural characterization data are as follows:

MS m/z (ESI): 959.3 [M+H]⁺

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

### Example 8: N—(((S)-1-(((S)-6-Amino-1-((2-((((1S,9S)-5-chloro-9-ethyl-9-hydroxy-4-methyl-10,13-dioxo-2,3,9,10,13,15-hexahydro))-1H,12H-benzo[de]pyrano[3′,4′:6,7]indolizino[1,2-b]quinolin-1-yl)amino)-2-oxoethoxy)methyl)amino)-1-oxohexan-2-yl)amino)-3-methyl-1-oxobutan-2-yl)-6-(2-(methylsulfonyl)pyrimidin-5-yl)hex-5-ynamide (A-52)

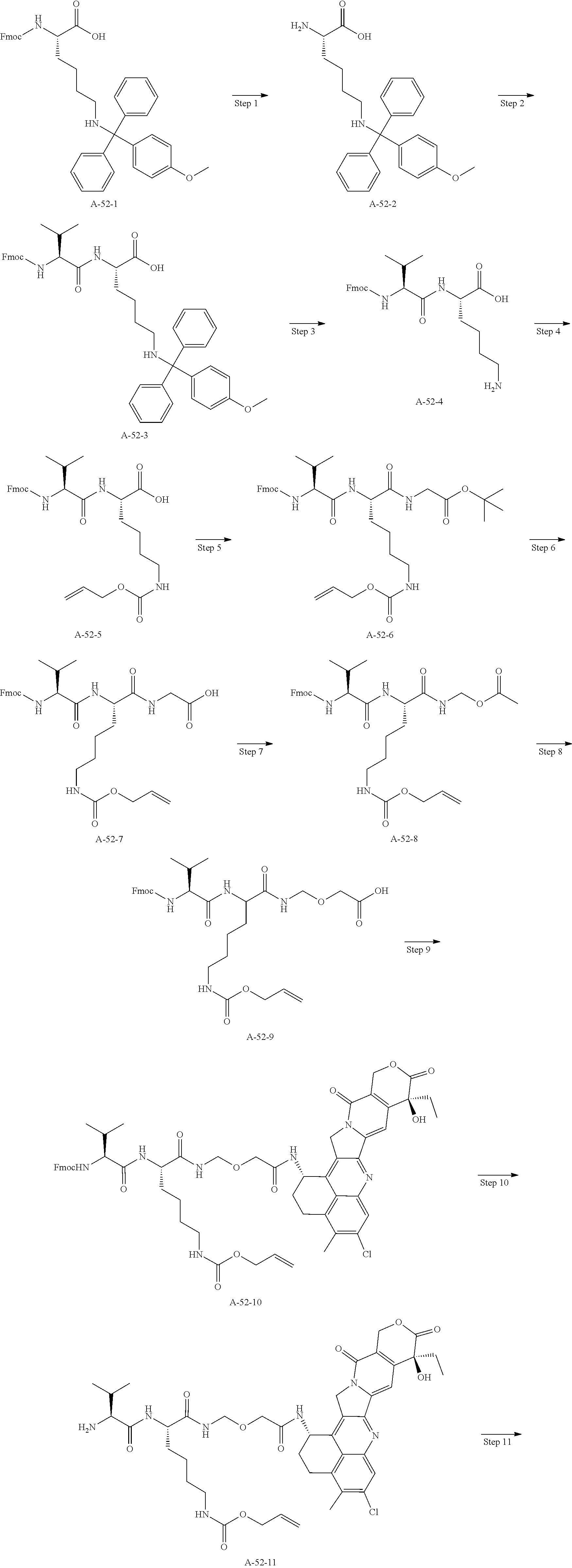

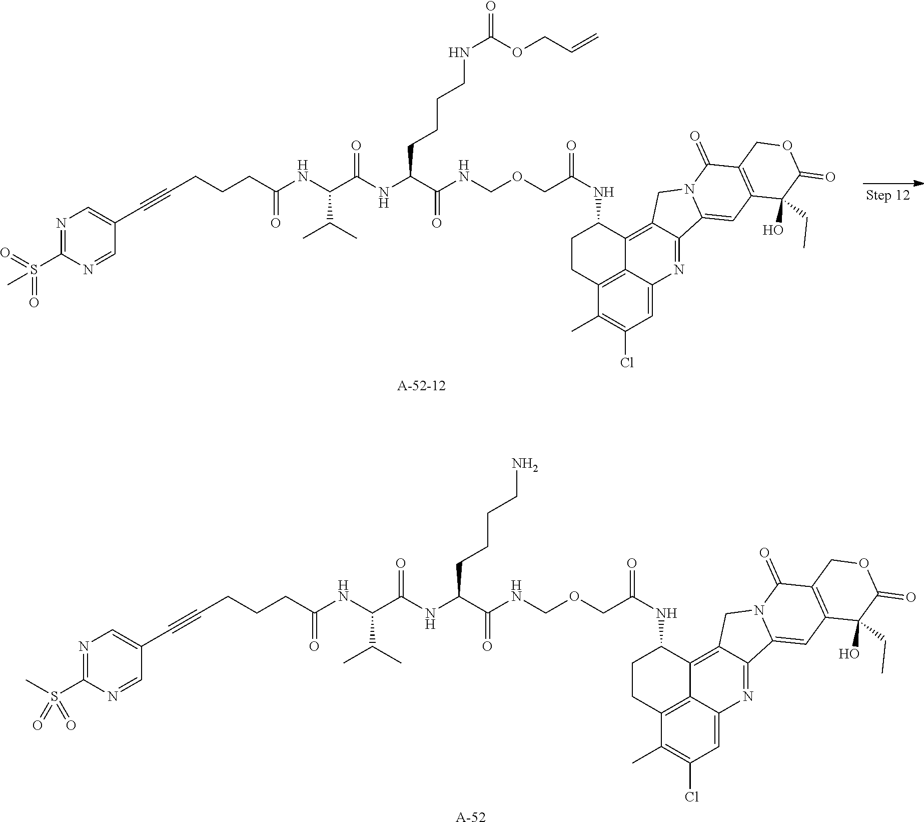 [SMILES: C=CCOC(=O)NCCCC[C@H](NC(=O)[C@@H](NC(=O)CCCC#Cc1cnc(S(C)(=O)=O)nc1)C(C)C)C(=O)NCOCC(=O)N[C@H]1CCc2c(C)c(Cl)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(Cl)c(C)c3c1c2[C@@H](NC(=O)COCNC(=O)[C@H](CCCCN)NC(=O)[C@@H](NC(=O)CCCC#Cc1cnc(S(C)(=O)=O)nc1)C(C)C)CC3 ; "A-52"]

### Step 1: Preparation of N⁶-((4-methoxyphenyl)diphenylmethyl)-L-lysine (A-52-2)

N²-((9H-Fluoren-9-yl)methoxy)carbonyl)-N⁶-((4-methoxyphenyl)diphenylmethyl)-L-lysine (10.0 g, 15 mmol) was dissolved in MeCN (50.0 mL), added with diethylamine (5.86 g, 15.61 mmol, 10.0 mL), and then stirred at 25° C. for 1 hour. The reaction mixture was directly concentrated to obtain a crude product, which was purified by silica gel column chromatography (petroleum ether/ethyl acetate=2:1; 100 mL×2) and then concentrated again to obtain the title compound (5.50 g, 13.1 mmol).

Its structural characterization data are as follows:

MS m/z (ESI): 373.2 [M-MMT+H]⁺

### Step 2: Preparation of N²-(((9H-fluoren-9-yl)methoxy)carbonyl)-L-valine)-N⁶-((4-methoxyphenyl)diphenylmethyl)-L-lysine (A-52-3)

2,5-Dioxopyrrolidin-1-yl ((9H-fluoren-9-yl)methoxy)carbonyl)-L-valine (6.88 g, 15.8 mmol) and (N⁶-((4-methoxyphenyl)diphenylmethyl)-L-lysine (5.50 g, 13.1 mmol) were dissolved in dichloromethane (100 mL), added with triethylamine (2.66 g, 26.3 mmol, 3.66 mL), and then stirred at 25° C. for 1 hour. The reaction solution was adjusted to pH 3-4 with 1N dilute hydrochloric acid, and extracted with dichloromethane three times (60.0 mL×3). The organic phases were combined, dried over anhydrous sodium sulfate, filtered and concentrated to obtain a crude product. The crude product was purified by silica gel column (dichloromethane/methanol=100/1 to 10/1), and then concentrated again to obtain the title compound (10.1 g, 12.3 mmol).

Its structural characterization data are as follows:

MS m/z (ESI): 738.3 [M−H]⁻

### Step 3: Preparation of (((9H-fluoren-9-yl)methoxy)carbonyl)-L-valine-L-lysine (A-52-4)

N²-(((9H-Fluoren-9-yl)methoxy)carbonyl)-L-valine)-N⁶-((4-methoxyphenyl)diphenylmethyl)-L-lysine (2.50 g, 3.38 mmol) was dissolved in dichloromethane (20.0 mL), added with a solution of hydrogen chloride in 1,4-dioxane (4 M, 7.00 mL), and stirred at 25° C. for 2 hours. The reaction solution was directly concentrated to obtain a crude product, which was purified by silica gel column (petroleum ether/ethyl acetate=1:1; 50.0 mL×2), and then concentrated again to obtain the hydrochloride of the title compound (1.80 g, 3.02 mmol).

Its structural characterization data are as follows:

MS m/z (ESI): 466.2 [M−H]⁻

### Step 4: Preparation of N²-(((9H-fluoren-9-yl)methoxy)carbonyl)-L-valine)-N⁶-((allyloxy) carbonyl)-L-lysine (A-52-5)

(((9H-Fluoren-9-yl)methoxy)carbonyl)-L-valine-L-lysine (1.6 g, 2.69 mmol) and allyl chloroformate (971 mg, 8.06 mmol, 855 μL) were dissolved in 1,4-dioxane (30.0 mL), added with 5% potassium carbonate aqueous solution, then stirred at 25° C. for half an hour. The reaction solution was adjusted to pH 3 with 1N dilute hydrochloric acid, then added with water (100 mL), and extracted with ethyl acetate three times (60.0 mL×3). The organic phases were combined and dried over anhydrous sodium sulfate, filtered and concentrated to obtain a crude product, which was purified by silica gel column (dichloromethane/methanol=100/1 to 20/1), and concentrated again to obtain the title compound (1.2 g, 2.04 mmol).

Its structural characterization data are as follows:

MS m/z (ESI): 552.2 [M+H]⁺

### Step 5: Preparation of tert-butyl N²-(((9H-fluoren-9-yl)methoxy)carbonyl)-L-valine)-N⁶-((allyloxy)carbonyl)-L-lysine-L-glycinate (A-52-6)

N²-(((9H-Fluoren-9-yl)methoxy)carbonyl)-L-valine)-N⁶-((allyloxy)carbonyl)-L-lysine (1.20 g, 2.18 mmol) and tert-butyl glycinate (342 mg, 2.61 mmol) were dissolved in DMF (20.0 mL), added with HOBT (881 mg, 6.53 mmol), EDCI (1.25 g, 6.53 mmol) and DIPEA (1.12 g, 8.70 mmol, 1.52 mL), and then stirred at 25° C. for 2 hours. The reaction solution was added with water (100 mL), and extracted with ethyl acetate 3 times (70.0 mL×3). The organic phases were combined and dried over anhydrous sodium sulfate, filtered and concentrated to obtain a crude product, which was purified by silica gel column (dichloromethane/methanol=100/1 to 20/1), and concentrated again to obtain the title compound (1.10 g, 1.49 mmol).

Its structural characterization data are as follows:

MS m/z (ESI): 665.2 [M+H]⁺

### Step 6: Preparation of N²-(((9H-fluoren-9-yl)methoxy)carbonyl)-L-valine)-N⁶-((allyloxy)carbonyl)-L-lysine-L-glycine (A-52-7)

Tert-butyl N²-(((9H-fluoren-9-yl)methoxy)carbonyl)-L-valine)-N⁶-((allyloxy)carbonyl)-L-lysine-L-glycinate (1.00 g, 1.50 mmol) was dissolved in dichloromethane (30.0 mL), added with trifluoroacetic acid (12.3 g, 107 mmol, 8.00 mL), and stirred at 25° C. for 3 hours. The reaction solution was directly concentrated to obtain the title compound as crude product (1.10 g), which was used directly in the next step without purification.

Its structural characterization data are as follows:

MS m/z (ESI): 609.2 [M+H]⁺

### Step 7: Preparation of (5S,8S)-8-(4-(allyloxy)carbonyl)amino)butyl)-1-(9H-fluoren-9-yl)-5-isopropyl-3,6,9-trioxo-2-oxa-4,7,10-triazaundecan-11-yl acetate (A-52-8)

N²-(((9H-Fluoren-9-yl)methoxy)carbonyl)-L-valine)-N⁶-((allyloxy)carbonyl)-L-lysine-L-glycine (910 mg, 1.50 mmol) was dissolved in DMF (15.0 mL), added with lead tetraacetate (795 mg, 1.79 mmol), AcOH (197 mg, 3.29 mmol, 188 μL) and copper acetate (108 mg, 598 μmol), heated to 60° C. and stirred for 2 hours. The reaction solution was added with water (100 mL), and extracted with ethyl acetate 3 times (70.0 mL×3). The organic phases were combined and dried over anhydrous sodium sulfate, filtered and concentrated to obtain a crude product, which was purified by silica gel column (petroleum ether/ethyl acetate=100/1 to 1/1) and concentrated again to obtain the title compound (890 mg, 1.02 mmol).

Its structural characterization data are as follows:

MS m/z (ESI): 645.4 [M+Na]⁺

### Step 8: Preparation of (5S)-8-(4-(((allyloxy)carbonyl)amino)butyl)-1-(9H-fluoren-9-yl)-5-isopropyl-3,6,9-trioxo-2,12-dioxa-4,7,10-triazatetradecan-14-oic acid (A-52-9)

(5S,8S)-8-(4-(Allyloxy)carbonyl)amino)butyl)-1-(9H-fluoren-9-yl)-5-isopropyl-3,6,9-trioxo-2-oxa-4,7,10-triazaundecan-11-yl acetate (880 mg, 1.41 mmol) and 2-hydroxyacetic acid (214 mg, 2.83 mmol, 172 μL) were dissolved in dichloromethane (10.0 mL), added with 4-methylbenzenesulfonic acid pyridine (284 mg, 1.13 mmol), and stirred at 50° C. for 12 hours. The reaction solution was concentrated under nitrogen atmosphere to obtain a crude product, which was purified by preparative high performance liquid chromatography and freeze-dried to obtain the title compound (250 mg, 387 μmol).

Its structural characterization data are as follows:

MS m/z (ESI): 656.4 [M+H₂O+H]⁺

Its separation and purification method was as follows

Chromatographic column: Phenomenex luna (10 μm × 25 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 40 | 60 | 36 |
| 8.00 | 70 | 30 | 36 |

### Step 9: Preparation of (9H-fluoren-9-yl)methyl ((S)-1-((((S,9S)-5-chloro-9-ethyl-9-hydroxy-4-methyl-10,13-dioxo-2,3,9,10,15-hexahydro-1H,12H-benzo[de]pyrano[3′,4′:6,7]indolizino[1,2-b]quinolin-1-yl)amino)-1,6,13-trioxo-3,14-dioxa-5,12-diazaheptadec-16-en-7-yl)amino)-3-methyl-1-oxobutan-2-yl)carbamate (A-52-10)

(5S)-8-(4-(((Allyloxy)carbonyl)amino)butyl)-1-(9H-fluoren-9-yl)-5-isopropyl-3,6,9-trioxo-2,12-dioxa-4,7,10-triazatetradecan-14-oic acid (40 mg, 62.63 μmol), (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (34.32 mg, 62.63 μmol), DIPEA (40.47 mg, 313.13 μmol) and DMTMM (36.92 mg, 125.25 μmol) were added to DMF (2 mL) in sequence. After the addition was completed, the mixture was stirred at room temperature for 1 hour. After the reaction was completed, the reaction solution was added with water (12 mL), and extracted with ethyl acetate (8 mL×3). The organic phases were combined and washed once with saturated brine (6 mL), then dried over anhydrous sodium sulfate, filtered and concentrated to obtain the title compound (63 mg, 55.21 μmol).

Its structural characterization data are as follows:

MS m/z (ESI): 1072.4 [M+H]⁺

### Step 10: Preparation of allyl (((S)-5-((S(2-amino-3-methylbutyramide))-6-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-6-oxohexyl)carbamate (A-52-11)

To DMF (2 mL), (9H-fluoren-9-yl)methyl ((S)-1-((((S,9S)-5-chloro-9-ethyl-9-hydroxy-4-methyl-10,13-dioxo-2,3,9,10,15-hexahydro-1H,12H-benzo[de]pyrano[3′,4′:6,7]indolizino[1,2-b]quinolin-1-yl)amino)-1,6,13-trioxo-3,14-dioxa-5,12-diazaheptadec-16-en-7-yl)amino)-3-methyl-1-oxobutan-2-yl)carbamate (63 mg, 94% purity, 55.21 μmol) and diethylamine (32 mg, 437 μmol) were added. After the addition, the mixture was stirred at room temperature for 1 hour. After the reaction, the reaction mixture was added with petroleum ether (6 mL×3) for extraction, the liquids were separated, and the organic phase was diluted with water (4 mL) and freeze-dried to obtain the title compound (30 mg, 35.28 μmol).

Its structural characterization data are as follows:

MS m/z (ESI): 850.3 [M+H]⁺

### Step 11: Preparation of allyl ((S)-6-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-5-(S)-3-methyl-2-(6-(2-(methylsulfonyl)pyrimidin-5-yl)hex-5-ynamido)butyramido)-6-oxohexyl)carbamate (A-52-12)

To DMF (2 mL), allyl (((S)-5-((S(2-amino-3-methylbutyramido))-6-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-6-oxohexyl)carbamate (30 mg, 35.28 μmol), 2,5-dioxopyrrolidin-1-yl 6-(2-(methylsulfonyl)pyrimidin-5-yl)hex-5-ynoate (12.89 mg, 35.28 μmol) and DIPEA (9.12 mg, 70.56 μmol) were added, stirred at room temperature for 1 hour. The reaction solution was added with water (8 mL), then extracted with ethyl acetate 3 times (6 mL×3). The organic phases were combined and washed with saturated brine (6 mL) once, dried over anhydrous sodium sulfate, filtered and concentrated to obtain the title compound (38 mg, 31.07 μmol), which was used directly in the next step without purification.

Its structural characterization data are as follows:

MS m/z(ESI): 1100.4 [M+H]⁺

### Step 12: Preparation of N—(((S)-1-(((S)-6-amino-1-((2-((((1S,9S)-5-chloro-9-ethyl-9-hydroxy-4-methyl-10,13-dioxo-2,3,9,10,13,15-hexahydro))-1H,12H-benzo[de]pyrano[3′,4′:6,7]indolizino[1,2-b]quinolin-1-yl)amino)-2-oxoethoxy)methyl)amino)-1-oxohexan-2-yl)amino]-3-methyl-1-oxobutan-2-yl)-6-(2-(methylsulfonyl)pyrimidin-5-yl)hexan-5-ynamide (A-52)

To DCM (3 mL), allyl ((S)-6-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-5-(S)-3-methyl-2-(6-(2-(methylsulfonyl)pyrimidin-5-yl)hex-5-ynamido)butyramido)-6-oxohexyl)carbamate (20 mg, 18.17 μmol), tetratriphenylphosphine palladium (4.75 mg, 4.09 μmol), formic acid (20 μL) and N-methylmorpholine (40 μL) were added, and reacted at room temperature for 1 hour under nitrogen protection. After the reaction was completed, the system was concentrated to obtain a crude product. The crude product was purified by preparative high performance liquid chromatography and freeze-dried to obtain the title compound (13.41 mg, 12.66 μmol).

Its structural characterization data are as follows:

MS m/z (ESI): 1016.3 [M+H]⁺

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 15 | 85 | 28 |
| 2.00 | 15 | 85 | 28 |
| 20 | 80 | 20 | 28 |

### Example 9: N-((10S,13S)-10-(4-Aminobutyl)-1-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-14-methyl-1,6,9,12-tetraoxo-5,8,11-triazapentadecan-13-yl)-6-(2-(methylsulfonyl)pyrimidin-5-yl)hex-5-ynamide (A-54)

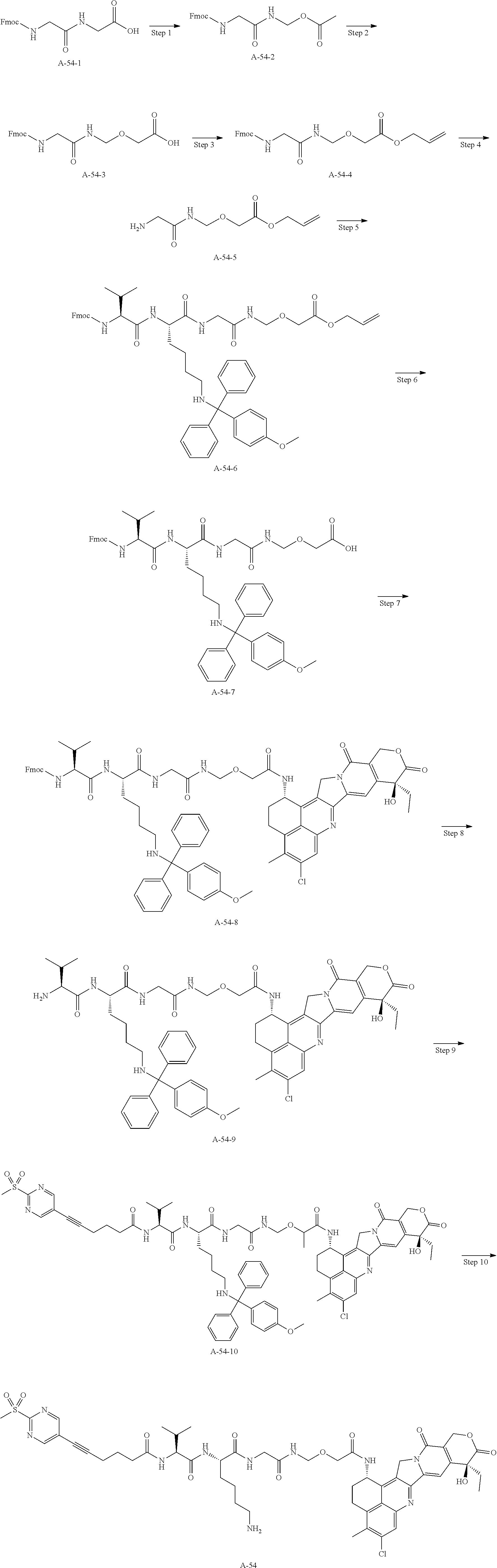

### Step 1: Preparation of methyl (2-((((9H-fluoren-9-yl)methoxy)carbonyl)amino) acetamido)acetate (A-54-2)

(((9H-Fluoren-9-yl)methoxy)carbonyl)glycylglycine (20.0 g, 56.5 mmol) was dissolved in DMF (200 mL), added with AcOH (7.46 g, 124 mmol, 7.10 mL), lead tetraacetate (27.5 g, 62.2 mmol) and copper acetate (4.10 g, 22.6 mmol), then heated to 60° C. and stirred for 2 hours. The reaction solution was added with water (500 mL), extracted with ethyl acetate 3 times (200 mL×3). The organic phases were combined and dried over anhydrous sodium sulfate, filtered and concentrated to obtain the title compound (16.0 g, 42.22 mmol).

### Step 2: Preparation of 1-(9H-fluoren-9-yl)-3,6-dioxo-2,9-dioxa-4,7-diazaundecan-11-oic acid (A-54-3)

Methyl (2-((((9H-fluoren-9-yl)methoxy)carbonyl)amino)acetamido)acetate (2.00 g, 5.43 mmol) was dissolved in dichloromethane (10.0 mL), added with PPTS (683 mg, 2.71 mmol) and glycolic acid (826 mg, 10.9 mmol, 661 μL), then heated to 43° C. and stirred for 4 hours. The reaction solution was filtered and concentrated to obtain a crude product, which was purified by silica gel column (dichloromethane/methanol=10/1) and concentrated again to obtain the title compound (1.48 g, 3.51 mmol).

Its structural characterization data are as follows:

MS m/z (ESI): 385.1 [M+H]⁺

### Step 3: Preparation of allyl 1-(9H-fluoren-9-yl)-3,6-dioxo-2,9-dioxa-4,7-diazaundecan-11-oate (A-54-4)

1-(9H-Fluoren-9-yl)-3,6-dioxo-2,9-dioxa-4,7-diazaundecan-11-oic acid (1.1 g, 2.86 mmol) was dissolved in DMF (10.0 mL), added with potassium carbonate (593 mg, 4.29 mmol) and 3-bromoprop-1-ene (692 mg, 5.72 mmol), and stirred at 25° C. for 6 hours. The reaction solution was cooled to room temperature, then added with water (10.0 mL), and extracted with ethyl acetate 3 times (10.0 mL×3). The organic phases were combined and washed with saturated sodium chloride aqueous solution (20 mL), dried over anhydrous sodium sulfate, filtered and concentrated to obtain 1.10 g of the crude product of the title compound.

Its structural characterization data are as follows:

MS m/z (ESI): 425.2 [M+H]⁺

### Step 4: Preparation of allyl 2-(2-aminoacetamido)methoxy)acetate (A-54-5)

Allyl 1-(9H-fluoren-9-yl)-3,6-dioxo-2,9-dioxa-4,7-diazaundecan-11-oate (1.10 g) was dissolved in DMF (10 mL), added with DBU (316 mg, 2.07 mmol, 313 μL), and stirred at 25° C. for 1 hour. The reaction solution was used directly in the next step without purification.

### Step 5: Preparation of allyl (5S,8S)-1-(9H-fluoren-9-yl)-5-isopropyl-8-(4-((4-methoxyphenyl)diphenylmethyl)amino)butyl)-3,6,9,12-tetraoxo-2,15-dioxa-4,7,10,13-tetraazaheptadecan-17-oate (A-54-6)

EDCI (746 mg, 3.89 mmol), HOBt (525 mg, 3.89 mmol) and N²-(((9H-fluoren-9-yl)methoxy)carbonyl)-L-valine)-N⁶-((4-methoxyphenyl)diphenylmethyl)-L-lysine (1.92 g, 2.59 mmol) were added to the DMF (10.0 mL) solution containing allyl 2-(2-aminoacetamido)methoxy)acetate (524 mg, 2.59 mmol) in the previous step, and then stirred at 25° C. for 1 hour. The reaction system was added with water (20.0 mL), and solid precipitated and was filtered. The filter cake was vacuum dried to obtain a crude product, which was purified by silica gel column (dichloromethane/methanol=20/1 to 10/1) and then concentrated again to obtain the title compound (1.20 g, 909 μmol).

Its structural characterization data are as follows:

MS m/z (ESI): 924.4 [M+H]⁺

### Step 6: Preparation of (5S,8S)-1-(9H-fluoren-9-yl)-5-isopropyl-8-(4-((4-methoxyphenyl) diphenylmethyl)amino)butyl)-3,6,9,12-tetraoxo-2,15-dioxa-4,7,10,13-tetraazaheptadecan-17-oic acid (A-54-7)

Allyl (5S,8S)-1-(9H-fluoren-9-yl)-5-isopropyl-8-(4-((4-methoxyphenyl)diphenylmethyl) amino)butyl)-3,6,9,12-tetraoxo-2,15-dioxa-4,7,10,13-tetraazaheptadecan-17-oate (1.20 g, 1.30 mmol) was dissolved in THF (10.0 mL), added with morpholine (226 mg, 2.60 mmol, 229 μL) and Pd(PPh₃)₄ (45.1 mg, 38.9 μmol), and stirred at 25° C. for 1 hour. The reaction solution was adjusted to pH 2 with 1N dilute hydrochloric acid, and extracted with dichloromethane 3 times (30.0 mL×3). The organic phases were combined and washed with saturated sodium chloride aqueous solution (50.0 mL), and dried over anhydrous sodium sulfate. The crude product was filtered and concentrated to obtain a crude product, which was purified by preparative high performance liquid chromatography and freeze-dried to obtain the title compound (358 mg, 388 μmol).

Its structural characterization data are as follows:

MS m/z (ESI): 884.4 [M+H]⁺

Its separation and purification method was as follows

Chromatographic column: Phenomenex luna (10 μm*50 mm × 250 mm)
Mobile phase A: acetonitrile; Mobile phase B: water
(0.05% ammonium bicarbonate)

| Time | Mobile phase A | Mobile phase B | Flow rate |
|---|---|---|---|
| [min] | [%] | [%] | [mL/min] |
| 0.00 | 40 | 60 | 36 |
| 10.00 | 70 | 30 | 36 |

### Step 7: Preparation of (9H-fluoren-9-yl)methyl ((10S,13S)-1-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-(4-((4-methoxyphenyl)diphenylmethyl)amino)butyl)-14-methyl-1,6,9,12-tetraoxo-3-oxa-5,8,11-triazapentadecan-13-yl)carbamate (A-54-8)

(5S,8S)-1-(9H-Fluoren-9-yl)-5-isopropyl-8-(4-((4-methoxyphenyl)diphenylmethyl)amino)butyl)-3,6,9,12-tetraoxo-2,15-dioxa-4,7,10,13-tetraazaheptadecan-17-oic acid (200 mg, 226.24 μmol), (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (130 mg, 237.55 μmol), DIPEA (146.20 mg, 1.13 mmol), and DMTMM (133.36 mg, 452.48 μmol) were added to DMF (3.5 mL) in sequence. After the addition, the mixture was stirred at room temperature for 1 hour. After the reaction was completed, the reaction solution was added with water (12 mL) and ethyl acetate (12 mL), and filtered through diatomaceous earth. The aqueous phase was extracted twice with ethyl acetate (12 mL×2), and the organic phases were combined and washed with saturated sodium chloride aqueous solution (6 mL), filtered and concentrated to obtain 324 mg of the crude product of the title compound.

Its structural characterization data are as follows:

MS m/z (ESI): 1317.6 [M+H]⁺

### Step 8: Preparation of (S)-2-((S)-2-amino-3-methylbutyramido)-N-(2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)2-oxoethyl)-6-((4-methoxyphenyl)diphenylmethyl)amino)hexanamide (A-54-9)

To DMF (3 mL), (9H-fluoren-9-yl)methyl ((10S,13S)-1-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-(4-((4-methoxyphenyl)diphenylmethyl)amino)butyl)-14-methyl-1,6,9,12-tetraoxo-3-oxa-5,8,11-triazapentadecan-13-yl)carbamate (278 mg, 210.94 μmol) and diethylamine (15 mg) were added, and stirred at room temperature for 1 hour. The reaction solution was added with petroleum ether (4 mL×2) for extraction, and the liquids were separated. The aqueous phase was concentrated under reduced pressure to remove diethylamine to obtain 180 mg of the crude product of the title compound.

Its structural characterization data are as follows:

MS m/z (ESI): 1095.5 [M+H]⁺

### Step 9: Preparation of N-((10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-(4-((((4-methoxyphenyl)diphenylmethyl)amino)butyl)-14-methyl-1,6,9,12-tetraoxo-3-oxa-5,8,11-triazapentadecan-13-yl)-6-(2-(methylsulfonyl)pyrimidin-5-yl)hex-5-ynamide (A-54-10)

To DMF (2 mL), (S)-2-((S)-2-amino-3-methylbutyramido)-N-(2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)2-oxoethyl)-6-((4-methoxyphenyl)diphenylmethyl)amino)hexanamide (160 mg, 146.03 μmol), 2,5-dioxopyrrolidin-1-yl 6-(2-(methylsulfonyl)pyrimidin-5-yl)hex-5-ynoate (70 mg, 191.59 μmol) and DIPEA (37.75 mg, 292.06 μmol) were added. After the addition, the mixture was stirred at room temperature for 1 hour. The reaction system was added with water (15 mL), and extracted with ethyl acetate twice (10 mL×2). The organic phase was washed once with saturated sodium chloride aqueous solution (6 mL), dried over anhydrous sodium sulfate, filtered and concentrated to obtain 156 mg of crude title compound.

Its structural characterization data are as follows:

MS m/z (ESI): 1359.5 [M+H]⁺

### Step 10: Preparation of N-((10S,13S)-10-(4-aminobutyl)-1-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-14-methyl-1,6,9,12-tetraoxa-5,8,11-triazapentadecan-13-yl)-6-(2-(methylsulfonyl)pyrimidin-5-yl)hex-5-ynamide (A-54)

To glacial acetic acid (2 mL) and water (0.5 mL), N-((10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-(4-((((4-methoxyphenyl)diphenylmethyl)amino)butyl)-14-methyl-1,6,9,12-tetraoxo-3-oxa-5,8,11-triazapentadecan-13-yl)-6-(2-(methylsulfonyl)pyrimidin-5-yl)hex-5-ynamide (140 mg, 104.02 μmol) was added. After the addition, the mixture was heated to 45° C. and reacted for 1 hour. The reaction mixture was concentrated to remove solvent, then directly purified by preparative high performance liquid chromatography and freeze-dried to obtain the title compound (72.72 mg, 58.78 μmol).

Its structural characterization data are as follows:

MS m/z (ESI): 1073.4 [M+H]⁺

Its separation and purification method was as follows:

Chromatographic column: Waters SunFire Prep
C18 OBD (5 μm*19 mm*150 mm)
Mobile phase A: acetonitrile; Mobile phase B: water
(0.05% trifluoroacetic acid)

| Time | Mobile phase A | Mobile phase B | Flow rate |
|---|---|---|---|
| [min] | [%] | [%] | [mL/min] |
| 0.00 | 10 | 90 | 28 |
| 4.00 | 10 | 90 | 28 |
| 24 | 90 | 10 | 28 |

### Example 10: (S)-5-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-4-((S)-2-((R)-4-methyl-2-(6-(2-(methylsulfonyl)pyrimidin-5-yl)hex-5-ynamino)pentanamido)propanamido)-5-oxopentanoic acid (A-62)

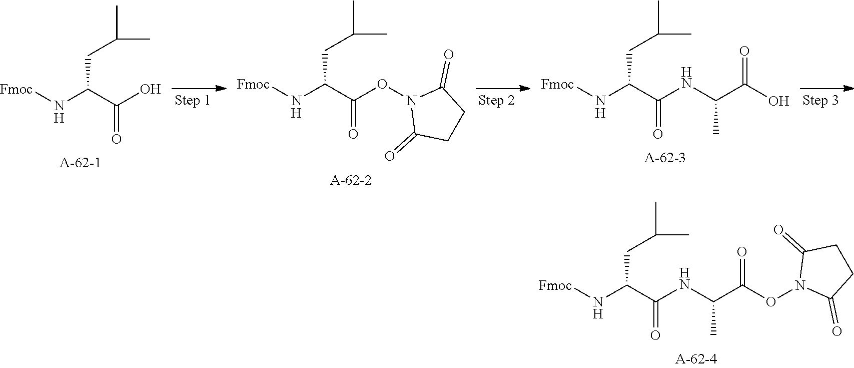

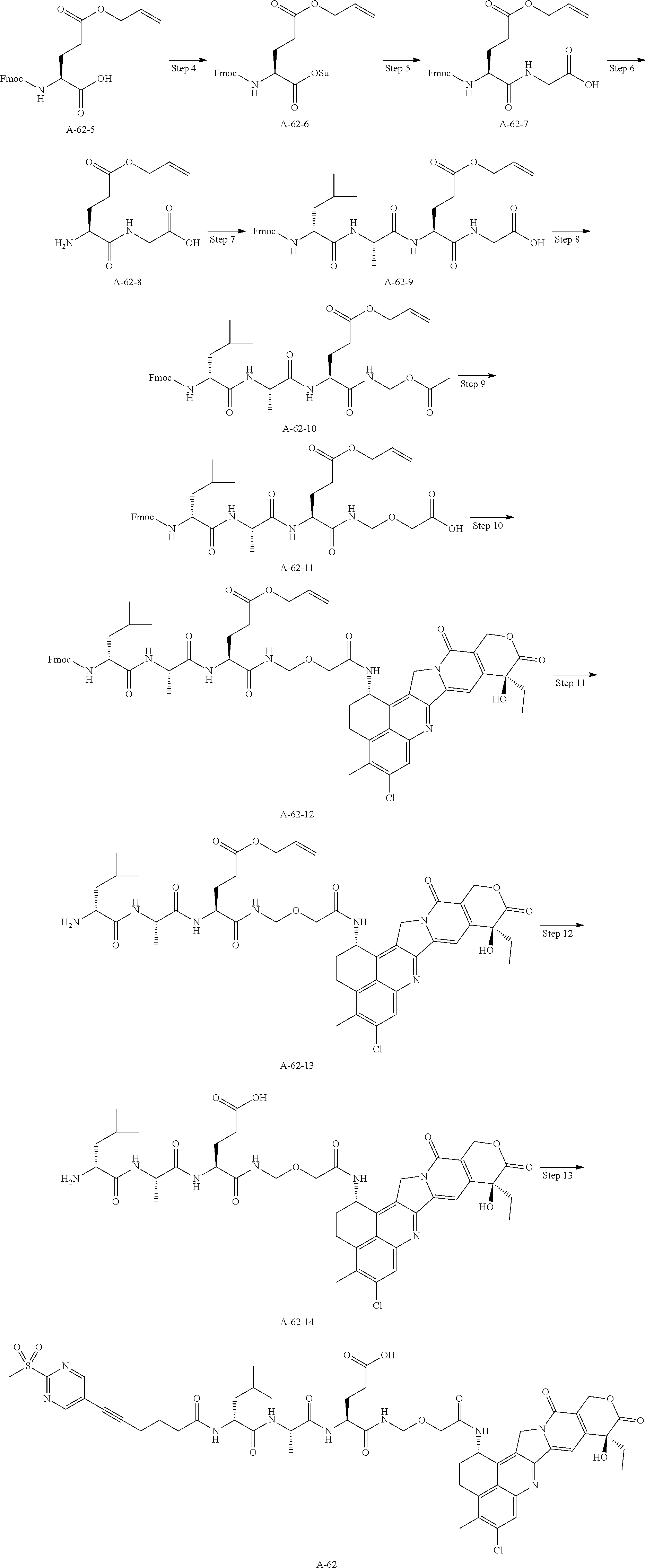

### Step 1: Preparation of 2,5-dioxopyrrolidin-1-yl (((9H-fluoren-9-yl)methoxy)carbonyl)-D-leucine (A-62-2)

(((9H-Fluoren-9-yl)methoxy)carbonyl)-D-leucine (1.5 g, 4.24 mmol) was dissolved in THF (15 mL), added with HOSu (586.16 mg, 5.09 mmol) and DCC (1.05 g, 5.09 mmol), and stirred at room temperature for about 1 hour. The system gradually became turbid and white solid precipitated. After the reaction was completed, the reaction solution was filtered, the filter cake was washed with a small amount of THF, and then concentrated under reduced pressure to obtain 1.91 g of the crude product of the title compound, which was directly used in the next step without purification.

Its structural characterization data are as follows:

MS m/z (ESI): 468.3 [M+H+H₂O]⁺, 923.3 [2M+Na]+

### Step 2: Preparation of ((9H-fluoren-9-yl)methoxy)carbonyl)-D-leucine-L-alanine (A-62-3)

5-Dioxopyrrolidin-1-yl (((9H-fluoren-9-yl)methoxy)carbonyl)-D-leucine (3.1 g, 6.88 mmol) and L-alanine (735.71 mg, 8.26 mmol) were dissolved in acetone (50 mL) and water (25 mL), added with NaHCO₃ (1.16 g, 13.76 mmol) at room temperature, and then stirred at room temperature for 1 hour. After the reaction was completed, the reaction solution was added with water, and then adjusted to pH 3-4 with 2N hydrochloric acid. The system was a milky white slurry, added with ethyl acetate (20 mL×2) for extraction twice. The organic phase was washed with brine and concentrated to obtain 2.92 g of the crude product of the title compound, which was used directly in the next reaction without purification.

Its structural characterization data are as follows:

MS m/z (ESI): 425.3 [M+H]⁺

### Step 3: Preparation of 2,5-dioxopyrrolidin-1-yl ((9H-fluoren-9-yl)methoxy)carbonyl)-D-leucine-L-alanine (A-62-4)

((9H-Fluoren-9-yl)methoxy)carbonyl)-D-leucine-L-alanine (2.92 g, 6.88 mmol) and HOSu (950.00 mg, 8.25 mmol) were dissolved in THF (30 mL), and added with DCC (1.70 g, 8.25 mmol) in batches, and white solid gradually precipitated in the system. After the reaction was completed, the reaction solution was filtered, the filter cake was washed with a small amount of THF, and then concentrated under reduced pressure to obtain 4.76 g of the crude product of the title compound, which was directly used in the next reaction without purification.

Its structural characterization data are as follows:

MS m/z (ESI): 439.3 [M+H+H₂O]⁺

### Step 4: Preparation of 5-allyl-1-(2,5-dioxopyrrolidin-1-yl) (((9H-fluoren-9-yl)methoxy)carbonyl)-L-glutamate (A-62-6)

HOSu (674.62 mg, 5.86 mmol), (S)-2-((((9H-fluoren-9-yl)methoxy)carbonyl)amino)-5-(allyloxy)-5-oxopentanoic acid (2 g, 4.88 mmol) were dissolved in THF (20 mL), added with DCC (1.21 g, 5.86 mmol) at room temperature, and then stirred at room temperature for 1 hour. After the reaction was completed, the reaction liquid was filtered, the filter cake was washed with a small amount of THF, and then concentrated under reduced pressure to obtain 2.47 g of the crude product of the title compound, which was directly used in the next reaction without purification.

Its structural characterization data are as follows:

MS m/z (ESI): 524.3 [M+H+H₂O]⁺

### Step 5: Preparation of (S)-(2-(((9H-fluoren-9-yl)methoxy)carbonyl)amino)-5-(allyloxy)-5-oxopentanoyl)glycine (A-62-7)

5-Allyl-1-(2,5-dioxopyrrolidin-1-yl) (((9H-fluoren-9-yl)methoxy)carbonyl)-L-glutamate (2.47 g, 4.88 mmol) and glycine (732.13 mg, 9.75 mmol) were dissolved in water (20 mL) and acetone (40 mL), added with NaHCO₃ (819.33 mg, 9.75 mmol) at room temperature, and then stirred at room temperature for 1 hour. After the reaction was completed, the reaction solution was added with water, and then adjusted to pH 3-4 with 2N hydrochloric acid. The system was a milky white slurry, and extracted with ethyl acetate twice (50 mL×2). The organic phases were combined, washed with brine, and concentrated to obtain 2.27 g of the crude product of the title compound, which was used directly in the next step without purification.

Its structural characterization data are as follows:

MS m/z (ESI): 467.3 [M+H]⁺

### Step 6: Preparation of (S)-(5-(allyloxy)-2-amino-5-oxopentanoyl)glycine (A-62-8)

(S)-(2-(((9H-Fluoren-9-yl)methoxy)carbonyl)amino)-5-(allyloxy)-5-oxopentanoyl)glycine (2.27 g, 4.87 mmol) was dissolved in DMF (20 mL), added with diethylamine (2 mL), and stirred at room temperature for 1 hour. After the reaction was completed, the reaction solution was concentrated under reduced pressure to obtain 1.19 g of the crude product of the title compound, which was directly used in the next reaction without purification.

Its structural characterization data are as follows:

MS m/z (ESI): 245.1 [M+H]⁺

### Step 7: Preparation of ((S)-2-((S)-2-((R)-2-(4-(9H-fluoren-9-yl)methoxy)carbonyl)amino)-4-methylpentanamido)propionamido)-5-(allyloxy)-5-oxopentanoyl)glycine (A-62-9)

2,5-Dioxopyrrolidin-1-yl ((9H-fluoren-9-yl)methoxy)carbonyl)-D-leucine-L-alaninate (3.07 g, 5.90 mmol) and (S)-(5-(allyloxy)-2-amino-5-oxopentanoyl)glycine (1.2 g, 4.91 mmol) were dissolved in DMF (30 mL), added with DIPEA (1.27 g, 9.83 mmol), and then stirred at room temperature for 1 hour. After the reaction was completed, the reaction solution was added with 60 mL of water, and extracted with ethyl acetate 3 times (30 mL×3). The ethyl acetate extraction solution was spin-dried, added with 30 mL of methyl tert-butyl ether, and filtered to remove the insoluble matter. The filtrate was concentrated to dryness. The crude product was purified by silica gel column chromatography (ethyl acetate/petroleum ether=0% to 100%) and concentrated again to obtain the title compound (2.8 g, 4.30 mmol).

Its structural characterization data are as follows:

MS m/z (ESI): 651.3 [M+H]⁺

### Step 8: Preparation of allyl (5R,8S,11S)-11-((acetoxymethyl)carbamoyl)-1-(9H-fluoren-9-yl)-5-isobutyl-8-methyl-3,6,9-trioxo-2-oxa-4,7,10-triazatetradecan-14-oic acid (A-62-10)

((S)-2-((S)-2-((R)-2-(4-(9H-Fluoren-9-yl)methoxy)carbonyl)amino)-4-methylpentanamido)propanamido)-5-(allyloxy)-5-oxopentanoyl)glycine (2.8 g, 4.30 mmol) was dissolved in THF (30 mL) and toluene (10 mL), then added with pyridine (1.02 g, 12.91 mmol) and lead tetraacetate (5.72 g, 12.91 mmol), subjected to nitrogen replacement, then heated to 85° C. and refluxed for 16 hours. After the reaction was completed, the reaction solution was cooled to room temperature, and then filtered to remove white solid. The filtrate was added with 40 mL of water and 20 mL of ethyl acetate, and no solid precipitated. Then 20 mL of saturated brine was added, and a large amount of white solid precipitated. The solid was removed by filtration. The filtrate was separated and extracted with ethyl acetate 3 times (20 mL×3). The organic phases were combined and dried over anhydrous sodium sulfate, and concentrated under reduced pressure to obtain a crude product. The crude product was purified by silica gel column chromatography (ethyl acetate/petroleum ether=0% to 50%) and concentrated again to obtain the title compound (720 mg, 1.08 mmol).

Its structural characterization data are as follows:

MS m/z (ESI): 684.4 [M+H+H₂O]⁺, 687.4 [M+Na]⁺

### Step 9: Preparation of (5R,8S,11S)-11-(3-(allyloxy)-3-oxopropyl)-1-(9H-fluoren-9-yl)-5-isobutyl-8-methyl-3,6,9,12-tetraoxo-2,15-dioxa-4,7,10,13-tetraazaheptadecan-17-oic acid (A-62-11)

Allyl (5R,8S,11S)-11-((acetoxymethyl)carbamoyl)-1-(9H-fluoren-9-yl)-5-isobutyl-8-methyl-3,6,9-trioxo-2-oxa-4,7,10-triazatetradecan-14-oate (720 mg, 1.08 mmol) and glycolic acid (247.12 mg, 3.25 mmol) were dissolved in THF. (10 mL), added with TsOH (37.30 mg, 216.62 μmol), then heated to 45° C. and stirred for 16 hours. After the reaction was completed, the reaction solution was concentrated to dryness, then added with 5 mL of methanol, and filtered to remove insoluble matter. The filtrate was concentrated, and purified by reverse phase column (acetonitrile/0.05% ammonium bicarbonate aqueous solution=0% to 50%) to obtain the title compound (230 mg, 337.86 μmol).

Its structural characterization data are as follows:

MS m/z (ESI): 698.3 [M+H+H₂O]⁺

### Step 10: Preparation of Allyl (5R,8S,11S)-11-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)carbamoyl)-1-(9H-fluoren-9-yl)-5-isobutyl-8-methyl-3,6,9-trioxo-2-oxa-4,7,10-triazatetradecan-14-oic acid (A-62-12)

(5R,8S,11S)-11-(3-(allyloxy)-3-oxopropyl)-1-(9H-fluoren-9-yl)-5-isobutyl-8-methyl-3,6,9,12-tetraoxo-2,15-dioxa-4,7,10,13-tetraazaheptadecan-17-oic acid (37.27 mg, 54.74 μmol), (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (30 mg, 54.74 μmol) were dissolved in DMF (0.5 mL), added with DIPEA (42.45 mg, 328.46 μmol), then added with DMTMM (48.40 mg, 164.23 μmol), and stirred at room temperature for 1 hour. After the reaction was completed, the reaction solution was used directly in the next reaction without treatment.

Its structural characterization data are as follows:

MS m/z (ESI): 1114.3 [M+H]⁺

### Step 11: Preparation of allyl (S)-4-((S)-2-((R)-2-amino-4-methylpentanamido) propionamido)-5-((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolinyl)amino)-2-oxoethoxy)methyl)amino)-5-oxopentanoate formate (A-62-13)

The reaction mixture containing (5R,8S,11S)-11-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)carbamoyl)-1-(9H-fluoren-9-yl)-5-isobutyl-8-methyl-3,6,9-trioxo-2-oxa-4,7,10-triazatetradecan-14-oic acid in the previous step was transferred to a single-necked flask using dichloromethane (1 mL), then added with diethylamine (1 mL), and stirred at room temperature for 1 hour. After the reaction was completed, the reaction solution was evaporated under reduced pressure to remove diethylamine and dichloromethane. The residue was purified by reverse phase column chromatography (acetonitrile/0.05% formic acid aqueous solution=0% to 50%) and freeze-dried again to obtain the formate salt of the title compound (100 mg, 88.45 μmol).

Its structural characterization data are as follows:

MS m/z (ESI): 892.4 [M+H]⁺

### Step 12: Preparation of (S)-4-((S)-2-((R)-2-amino-4-methylpentanamido)propionamido)-5-(((2-((((1S,9S)-5-chloro-9-ethyl-9-hydroxy-4-methyl-10,13-dioxo-2,3,9,13,15-hexahydro-1H,12H-benzo[de]pyrano[3′,4′:6,7]indolizino[1,2-b]quinolin-1-yl)amino)-2-oxoethoxy)methyl)amino)-5-oxopentanoic acid formate (A-62-14)

Allyl (S)-4-((S)-2-((R)-2-amino-4-methylpentanamido)propionamido)-5-((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolinyl)amino)-2-oxoethoxy)methyl)amino)-5-oxopentanoate formate (71.31 mg, 79.91 μmol, formate) was dissolved in THF (6 mL), added with morpholine (13.92 mg, 159.83 μmol), subjected to nitrogen replacement, then added with Pd(PPh₃)₄ (9.23 mg, 7.99 μmol), subjected to nitrogen replacement again three times, and then stirred at room temperature for 5 hours. The reaction solution was directly concentrated and purified by preparative high performance liquid chromatography and freeze-dried to obtain the title compound (25 mg, 27.27 μmol).

Its structural characterization data are as follows:

MS m/z (ESI): 852.3 [M+H]⁺

Its separation and purification method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 10 | 90 | 30 |
| 2.00 | 10 | 90 | 30 |
| 18.00 | 80 | 20 | 30 |

### Step 13: Preparation of (S)-5-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-4-((S)-2-((R)-4-methyl-2-(6-(2-(methylsulfonyl)pyrimidin-5-yl)hex-5-ynamino)pentanamido)propanamido)-5-oxopentanoic acid (A-62)

(S)-4-((S)-2-((R)-2-Amino-4-methylpentanamido)propionamido)-5-(((2-((((1S,9S)-5-chloro-9-ethyl-9-hydroxy-4-methyl-10,13-dioxo-2,3,9,13,15-hexahydro-1H,12H-benzo[de]pyrano[3′,4′:6,7]indolizino[1,2-b]quinolin-1-yl)amino)-2-oxoethoxy)methyl)amino)-5-oxopentanoic acid formate (7 mg, 7.79 μmol) was dissolved in DMF (1 mL), added with orenDIPEA (2.01 mg, 15.58 μmol), then added with 2,5-dioxopyrrolidin-1-yl 6-(2-(methylsulfonyl)pyrimidin-5-yl) hex-5-ynoate (3.42 mg, 9.35 μmol), and stirred at room temperature for 4 hours. The reaction solution was directly purified by preparative high performance liquid chromatography and freeze-dried to obtain the title compound (6 mg, 5.17 μmol).

Its structural characterization data are as follows:

MS m/z (ESI): 1102.4 [M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 28 |
| 2.00 | 30 | 70 | 28 |
| 18.00 | 90 | 10 | 28 |

### Example 11: (S)-5-((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)2-oxoethyl)amino)-4-(2-(6-(2-(methylsulfonyl)pyrimidin-5-yl)hex-5-ynamino)acetamido)-5-oxopentanoic acid (A-66)

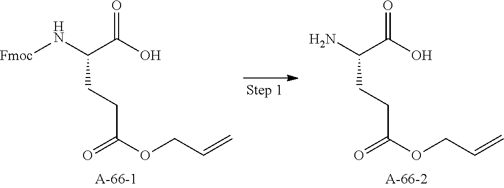

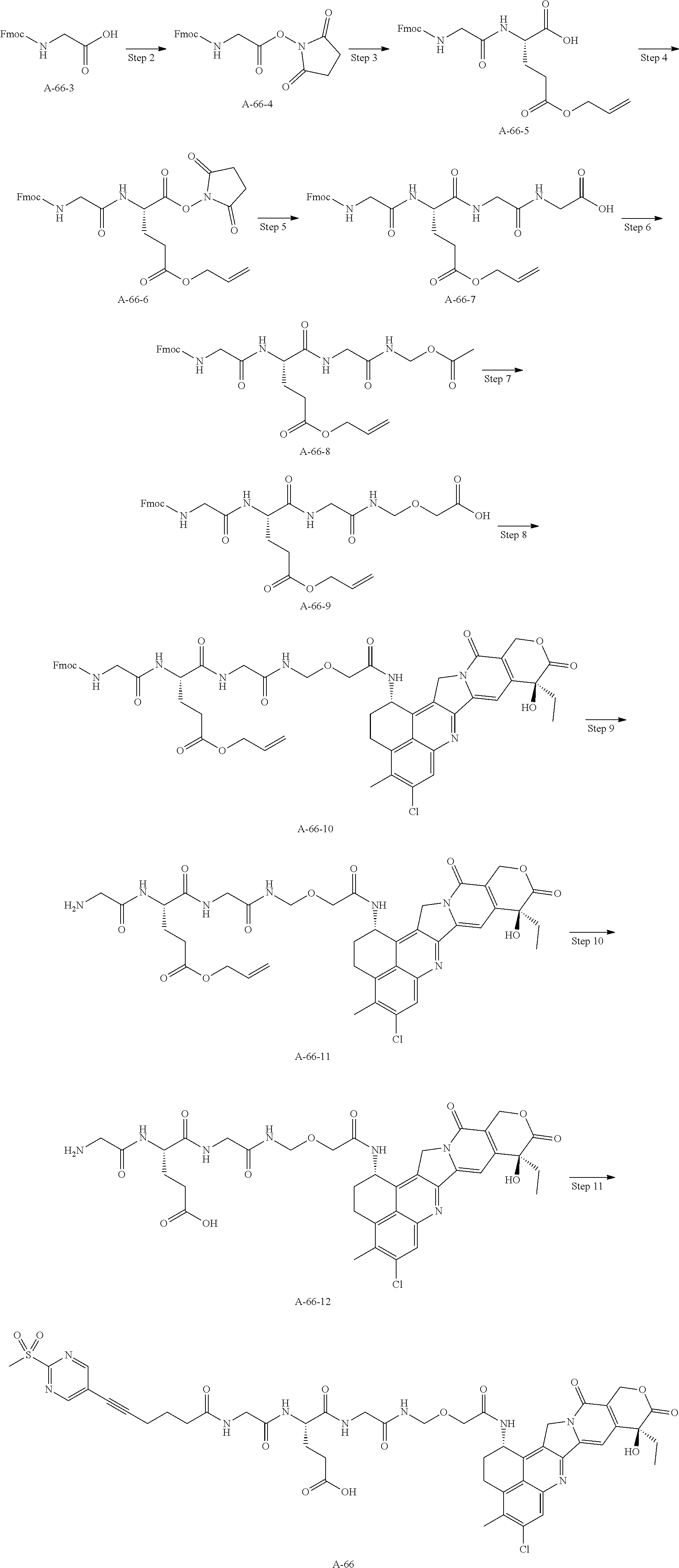

### Step 1: Preparation of (S)-5-(allyloxy)-2-amino-5-oxopentanoic acid (A-66-2)

(S)-2-((((9H-Fluoren-9-yl)methoxy)carbonyl)amino)-5-(allyloxy)-5-oxopentanoic acid (1.5 g, 3.66 mmol) was dissolved in DMF (15 mL), added with diethylamine (2 mL), and stirred at room temperature for 2 hours. After the reaction was completed, the diethylamine and DMF were removed by lyophilization to obtain the crude product of the title compound, which was directly used in the next reaction without purification.

Its structural characterization data are as follows:

MS m/z (ESI): 188.1 [M+H]⁺

### Step 2: Preparation of 2,5-dioxopyrrolidin-1-yl (((9H-fluoren-9-yl)methoxy)carbonyl)glycine (A-66-4)

((9H-Fluoren-9-yl)methoxy)carbonyl)glycine (1 g, 3.36 mmol) was dissolved in THF (30 mL), added with HOSu (425.81 mg, 3.70 mmol) and DCC (763.40 mg, 3.70 mmol), and stirred at room temperature for about 1 hour. The system gradually became turbid and white solid precipitated. After the reaction was completed, the reaction solution was filtered, the filter cake was washed with an appropriate amount of THF, and then concentrated under reduced pressure to obtain 1.27 g of the crude product of the title compound, which was directly used in the next step without purification.

Its structural characterization data are as follows:

MS m/z (ESI): 412.1 [M+H₂O+H]⁺

### Step 3: Preparation of (S)-2-(2-((((9H-fluoren-9-yl)methoxy)carbonyl)amino)acetylamino)-5-(allyloxy)-5-oxopentanoic acid (A-66-5)

(S)-5-(allyloxy)-2-amino-5-oxopentanoic acid (680 mg, 3.63 mmol) was dissolved in DMF (15 mL), added with DIPEA (469.49 mg, 3.63 mmol), then added with 2,5-dioxopyrrolidin-1-yl (((9H-fluoren-9-yl)methoxy)carbonyl)glycinate (1.43 g, 3.63 mmol), and stirred at room temperature for 2 hours. After the reaction was completed, the reaction solution was poured into 30 mL of water, adjusted to pH 3-4 with 2 N hydrochloric acid, and extracted with ethyl acetate 3 times (20 mL×3). The organic phases were combined, dried over anhydrous sodium sulfate, and concentrated under reduced pressure to obtain 1.5 g of the crude product of the title compound, which was used directly in the next step without purification.

Its structural characterization data are as follows:

MS m/z (ESI): 467.2 [M+H]⁺

### Step 4: Preparation of 5-allyl-1-(2,5-dioxopyrrolidin-1-yl) (((9H-fluoren-9-yl)methoxy)carbonyl)glycyl-L-glutamate (A-66-6)

(S)-2-(2-((((9H-Fluoren-9-yl)methoxy)carbonyl)amino)acetylamino)-5-(allyloxy)-5-oxopentanoic acid (1.69 g, 3.62 mmol) and HOSu (500.33 mg, 4.35 mmol) were suspended in dichloromethane (20 mL), added with DCC (896.99 mg, 4.35 mmol), and stirred at room temperature for 2 hours, and a large amount of insoluble matter appeared in the system. After the reaction was completed, the reaction solution was filtered to remove the insoluble matter, and the filtrate was concentrated under reduced pressure, purified by silica gel column chromatography (ethyl acetate/petroleum ether=20% to 60%), and then concentrated again to obtain 1.38 g of the title compound.

Its structural characterization data are as follows:

MS m/z (ESI): 564.2 [M+H]⁺, 591.3 [M+H₂O+H]⁺

### Step 5: Preparation of (S)-(2-((((9H-fluoren-9-yl)methoxy)carbonyl)amino)acetamido)-5-(allyloxy)-5-oxopentanoyl)glycylglycine (A-66-7)

Glycylglycine (154.73 mg, 1.17 mmol) was dissolved in acetone (3 mL), added with sodium bicarbonate (98.39 mg, 1.17 mmol), then added with 5-allyl-1-(2,5-dioxopyrrolidin-1-yl) (((9H-fluoren-9-yl)methoxy)carbonyl)glycyl-L-glutamate (330 mg, 585.57 μmol), and stirred at room temperature for 2 hours. After the reaction was completed, the reaction solution was poured into 10 mL of water, adjusted to pH 3-4 with 2 N dilute hydrochloric acid, and extracted with ethyl acetate 3 times (10 mL×3). The organic phases were combined, dried over anhydrous sodium sulfate, filtered and concentrated to obtain the title compound as a crude product (270 mg, 465.05 μmol), which was directly used in the next step without purification.

Its structural characterization data are as follows:

MS m/z (ESI): 581.3 [M+H]⁺

### Step 6: Preparation of allyl (S)-4-(2-((((9H-fluoren-9-yl)methoxy)carbonyl)amino) acetamide)-5-((2-(acetoxymethyl)amino)-2-oxoethyl)amino)-5-oxopentanoate (A-66-8)

(S)-(2-((((9H-Fluoren-9-yl)methoxy)carbonyl)amino)acetamide)-5-(allyloxy)-5-oxopentanoyl)glycylglycine (270 mg, 465.05 μmol) was dissolved in THF (3.0 mL) and toluene (1.0 mL), and added with pyridine (110.36 mg, 1.40 mmol) and lead tetraacetate (618.57 mg, 1.40 mmol). The system turned yellow, subjected to nitrogen replacement, and heated to 70° C. for 4 hours. After the reaction was completed, the reaction solution was added with water (5 mL), and extracted with ethyl acetate three times (5 mL×3). The combined organic phases were washed with saturated ammonium chloride (5 mL) and brine (5 mL) in sequence, dried over anhydrous sodium sulfate, filtered and concentrated to obtain a crude product, which was purified by silica gel column chromatography (ethyl acetate/petroleum ether=50% to 100%) and concentrated again to obtain the title compound (120 mg, 201.8 μmol).

Its structural characterization data are as follows:

MS m/z (ESI): 612.3 [M+H₂O+H]⁺

### Step 7: Preparation of allyl (S)-8-(3-(allyloxy)-3-oxopropyl)-1-(9H-fluoren-9-yl)-3,6,9,12-tetraoxo-2,15-dioxa-4,7,10,13-tetraazaheptadecan-17-oic acid (A-66-9)

Allyl (S)-4-(2-((((9H-fluoren-9-yl)methoxy)carbonyl)amino)acetamide)-5-((2-(acetoxymethyl)amino)-2-oxoethyl)amino)-5-oxopentanoate (40 mg, 67.27 μmol) and glycolic acid (25.58 mg, 336.35 μmol) were dissolved in THF (1 mL), added with p-toluenesulfonic acid (1.16 mg, 6.73 μmol), and stirred at room temperature for 3 hours. After the reaction was completed, the reaction mixture was added with water (5 mL), and extracted with dichloromethane (5 mL×3). The organic phases were combined and washed twice with saturated brine (5 mL), dried over anhydrous sodium sulfate, filtered and concentrated to obtain the title compound as a crude product (30 mg, 39.30 μmol), which was directly used in the next step without purification.

Its structural characterization data are as follows:

MS m/z (ESI): 609.3 [M−H]⁻

### Step 8: Preparation of allyl (S)-4-(2-((((9H-fluoren-9-yl)methoxy)carbonyl)amino) acetamido)-5-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-5-oxopentanoate (A-66-10)

Allyl (S)-8-(3-(allyloxy)-3-oxopropyl)-1-(9H-fluoren-9-yl)-3,6,9,12-tetraoxo-2,15-dioxa-4,7,10,13-tetraazaheptadecan-17-oate (30 mg, 39.30 μmol) and (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (21.54 mg, 39.30 μmol) were dissolved in DMF (1.5 mL), added with DIPEA (30.48 mg, 235.83 μmol) and DMTMM (34.75 mg, 117.91 μmol), and stirred at room temperature for 0.5 hours. After the reaction was completed, the reaction solution was directly used in the next reaction without treatment.

Its structural characterization data are as follows:

MS m/z (ESI): 1045.4[M+H]⁺

### Step 9: Preparation of allyl (S)-4-(2-aminoacetamido)-5-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-5-oxopentanoate (A-66-11)

To the reaction mixture containing allyl (S)-4-(2-((((9H-fluoren-9-yl)methoxy)carbonyl) amino)acetamido)-5-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-5-oxopentanoate (41 mg, 39.25 μmol) in the previous step, DMF (1.5 mL) and ethylenediamine (0.5 mL) were added, and stirred at room temperature for 10 minutes. After the reaction was completed, diethylamine was removed by rotary evaporation under reduced pressure, and the residue was purified by reverse phase column chromatography (acetonitrile/1% formic acid aqueous solution=0% to 50%) and freeze-dried to obtain the title compound (20 mg, 24.32 μmol).

Its structural characterization data are as follows:

MS m/z (ESI): 840.3 [M+H₂O+H]⁺

### Step 10: Preparation of (S)-4-(2-aminoacetamide)-5-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-5-oxopentanoic acid (A-66-12)

Allyl (S)-4-(2-aminoacetamido)-5-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)2-oxoethyl)amino)-5-oxopentanoate (20 mg, 24.32 μmol) was dissolved in THF (1 mL), added with morpholine (2.12 mg, 24.32 μmol) and Pd(PPh₃)₄ (2.81 mg, 2.43 μmol), subjected to nitrogen replacement three times, and reacted under nitrogen protection for 2 hours. After the reaction was completed, the reaction solution was directly spin-dried under reduced pressure, then dissolved in DMF and filtered, and the filtrate was purified by preparative high performance liquid chromatography and freeze-dried to obtain the title compound (3 mg, 3.84 μmol).

Its structural characterization data are as follows:

MS m/z (ESI): 782.2 [M+H]⁺

Its separation and purification methods are as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 2.00 | 10 | 90 | 30 |
| 18.00 | 80 | 20 | 30 |

### Step 11: Preparation of (S)-5-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-4-(2-(6-(2-(methylsulfonyl)pyrimidin-5-yl)hex-5-ynamino)acetamido)-5-oxopentanoic acid (A-66)

(S)-4-(2-aminoacetamido)-5-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)2-oxoethyl)amino)-5-oxopentanoic acid (3 mg, 3.84 μmol) and 2,5-dioxopyrrolidin-1-yl 6-(2-(methylsulfonyl)pyrimidin-5-yl)hex-5-ynoate (1.40 mg, 3.84 μmol) were added to DMF (1 mL), then added with DIPEA (991.38 μg, 7.67 μmol), stirred at room temperature for 5 hours. After the reaction was complete, the reaction solution was added dropwise with formic acid until the system was weakly acidic and pH was 5-6, which was then purified by preparative high performance liquid chromatography and freeze-dried to obtain the title compound (0.7 mg, 0.67 μmol).

Its structural characterization data are as follows:

MS m/z (ESI): 1034.3[M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 28 |
| 2.00 | 20 | 80 | 28 |
| 16.00 | 80 | 20 | 28 |

### Example 12: (S)-5-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-4-(2-(6-(2-(methylsulfonyl)pyrimidin-5-yl)hex-5-ynylamino)acetamido)-5-oxopentanoic acid (A-68)

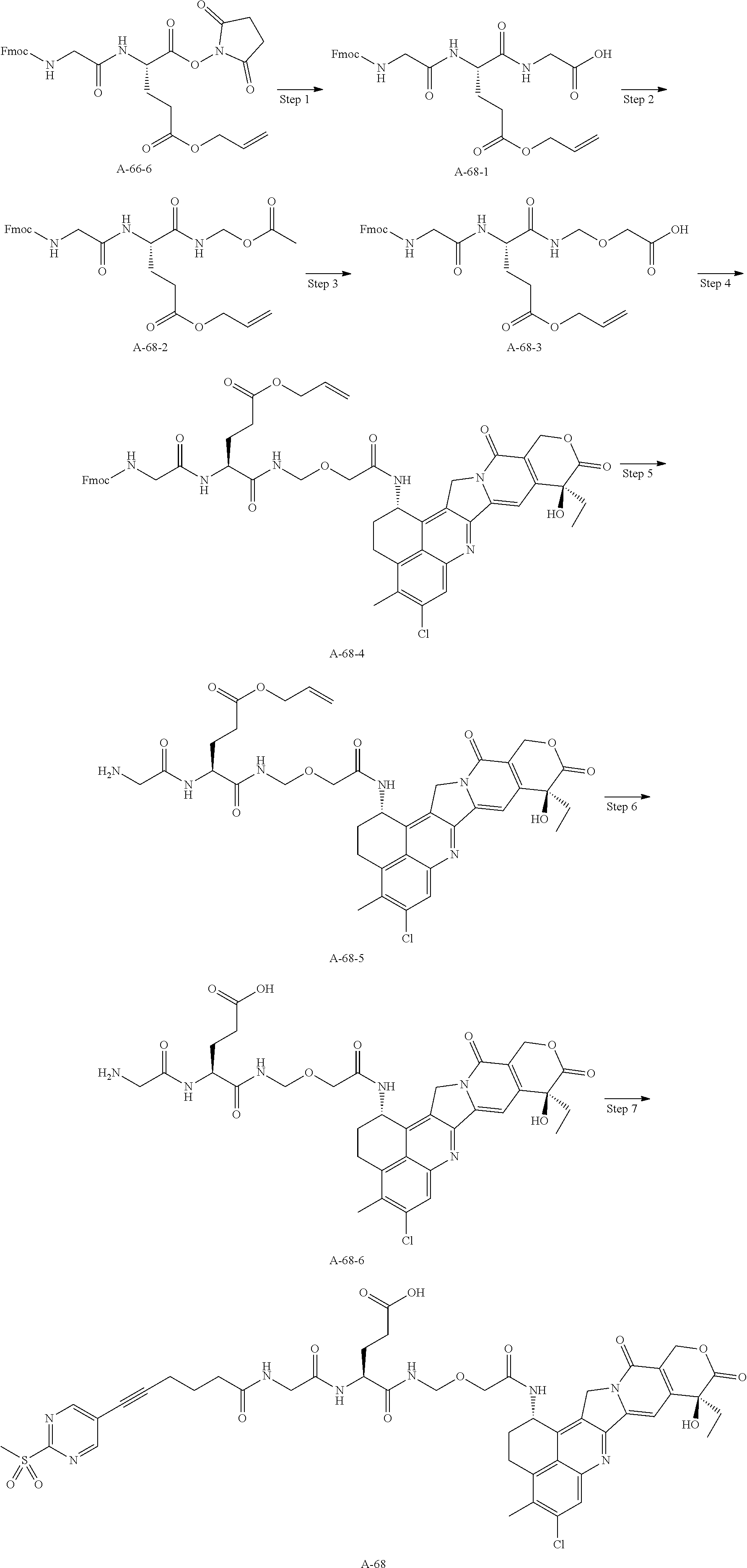

### Step 1: Preparation of (S)-(2-((((9H-fluoren-9-yl)methoxy)carbonyl)amino)acetamide)-5-(allyloxy)-5-oxopentanoyl)glycine (A-68-1)

Glycine (87.91 mg, 1.17 mmol) was dissolved in acetone (3 mL), added sodium bicarbonate (98.39 mg, 1.17 mmol), then added with 5-allyl-1-(2,5-dioxopyrrolidin-1-yl) (((9H-fluoren-9-yl)methoxy)carbonyl)glycyl-L-glutamate (330 mg, 585.57 μmol), and stirred at room temperature for 2 hours. After the reaction was completed, the reaction solution was poured into 10 mL of water, adjusted to pH 3-4 with 2N hydrochloric acid, and extracted with ethyl acetate 3 times (10 mL×3). The organic phases were combined, dried over anhydrous sodium sulfate, and concentrated under reduced pressure to obtain 230 mg of the crude product of the title compound, which was used directly in the next step without purification.

Its structural characterization data are as follows:

MS m/z (ESI): 524.3[M+H]⁺

### Step 2: Preparation of allyl (S)-4-(2-(3-(9H-fluoren-9-yl)methoxy)carbonyl)amino) acetamido)-5-((acetoxymethyl)amino)-5-oxopentanoate (A-68-2)

(S)-(2-((((9H-Fluoren-9-yl)methoxy)carbonyl)amino)acetamido)-5-(allyloxy)-5-oxopentanoyl)glycine (230 mg, 439.32 μmol) was dissolved in THF (3 mL) and toluene (1 mL), added with pyridine (104.25 mg, 1.32 mmol) and lead tetraacetate (584.35 mg, 1.32 mmol), subjected to nitrogen replacement, heated to 70° C. and reacted for 4 hours. After the reaction was completed, the reaction mixture was added with 5 mL of water, then extracted with ethyl acetate 3 times (5 mL×3). The organic phases were combined and washed with saturated ammonium chloride (5 mL) and brine (5 mL) in turn, dried over anhydrous sodium sulfate, filtered and concentrated to obtain a crude product, which was purified by silica gel column chromatography (ethyl acetate/petroleum ether=30% to 70%) to obtain the title compound (63 mg, 117.2 μmol).

Its structural characterization data are as follows:

MS m/z (ESI): 555.3 [M+H₂O+H]⁺

### Step 3: Preparation of (S)-8-(3-(allyloxy)-3-oxopropyl)-1-(9H-fluoren-9-yl)-3,6,9-trioxy-2,12-dioxy-4,7,10-triazatetradecan-14-oic acid (A-68-3)

Allyl (S)-4-(2-(3-(9H-fluoren-9-yl)methoxy)carbonyl)amino)acetamido)-5-((acetoxymethyl) amino)-5-oxopentanoate (30 mg, 55.81 μmol) and glycolic acid (21.22 mg, 279.04 μmol) were dissolved in THF (1 mL), added with p-toluenesulfonic acid (961.02 μg, 5.58 μmol), and stirred at room temperature for 3 hours. The reaction solution was added with water (5 mL), and then extracted with dichloromethane 3 times (5 mL×3). The organic phases were combined and washed twice with saturated brine (5 mL×2), dried over anhydrous sodium sulfate, filtered and concentrated to obtain the title compound as crude product (35 mg, 50.58 μmol), which was directly used in the next step without purification.

Its structural characterization data are as follows:

MS m/z (ESI): 552.2 [M−H]⁻

### Step 4: Preparation of allyl (S)-4-(2-aminoacetamide)-5-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-5-oxopentanoate (A-68-4)

(S)-8-(3-(Allyloxy)-3-oxopropyl)-1-(9H-fluoren-9-yl)-3,6,9-trioxy-2,12-dioxo-4,7,10-triazatetradecan-14-oic acid (35 mg, 50.58 μmol) and (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (27.72 mg, 50.58 μmol) were dissolved in DMF (1.5 mL), added with DIPEA (39.22 mg, 303.49 μmol) and DMTMM (44.72 mg, 151.75 μmol), and stirred at room temperature for 0.5 hours. After the reaction was completed, the reaction solution was directly used in the next reaction without treatment.

Its structural characterization data are as follows:

MS m/z (ESI): 987.2[M+H]⁺

### Step 5: Preparation of allyl (S)-4-(2-aminoacetamido)-5-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-5-oxopentanoate (A-68-5)

To the reaction solution containing allyl (S)-4-(2-aminoacetamido)-5-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-5-oxopentanoate (49 mg, 49.62 μmol) in the previous step, DMF (1.5 mL) and diethylamine (0.5 mL) were added, and stirred at room temperature for 10 minutes. After the reaction was completed, the reaction solution was directly purified by reverse phase column chromatography (acetonitrile/0.05% formic acid aqueous solution=0% to 50%) and freeze-dried to obtain the title compound (30 mg, 39.21 μmol).

Its structural characterization data are as follows:

MS m/z (ESI): 765.3[M+H]⁺

### Step 6: Preparation of (S)-4-(2-aminoacetamido)-5-((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-5-oxopentanoic acid (A-68-6)

Allyl (S)-4-(2-aminoacetamido)-5-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-5-oxopentanoate (30 mg, 39.21 μmol) was dissolved in THF (1 mL), added with morpholine (3.42 mg, 39.21 μmol) and Pd(PPh₃)₄ (4.53 mg, 3.92 μmol), subjected to nitrogen replacement three times, and reacted at room temperature under nitrogen protection for 2 hours. The reaction solution was concentrated to obtain a crude product, which was purified by preparative high performance liquid chromatography and freeze-dried to obtain the title compound (3 mg, 4.14 μmol).

Its structural characterization data are as follows:

MS m/z (ESI): 725.3[M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 2.00 | 10 | 90 | 30 |
| 18.00 | 80 | 20 | 30 |

### Step 7: Preparation of (S)-5-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-4-(2-(6-(2-(methylsulfonyl)pyrimidin-5-yl)hex-5-ynylamino)acetamido)-5-oxopentanoic acid (A-68)

(S)-4-(2-Aminoacetamido)-5-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-5-oxopentanoic acid (3 mg, 4.14 μmol) and 2,5-dioxopyrrolidin-1-yl 6-(2-(methylsulfonyl)pyrimidin-5-yl)hex-5-ynoate (1.51 mg, 4.14 μmol) were added to DMF (1 mL), then added with DIPEA (1.07 mg, 8.27 μmol), and stirred at room temperature for 5 hours. The reaction solution was added dropwise with formic acid until the system was weakly acidic (pH 5-6), and then purified by preparative high performance liquid chromatography and lyophilized to obtain the title compound (3.49 mg, 3.45 μmol).

Its structural characterization data are as follows:

MS m/z (ESI): 975.2[M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 28 |
| 2.00 | 20 | 80 | 28 |
| 16.00 | 80 | 20 | 28 |

### Example 13: (S)—N-((2-(((1S,9S)-5-Chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)-1-((6-(2-(methylsulfonyl)pyrimidin-5-yl)hex-5-ynyl)-L-serine-D-propylamino)pyrrolidine-2-carboxamide (A-72)

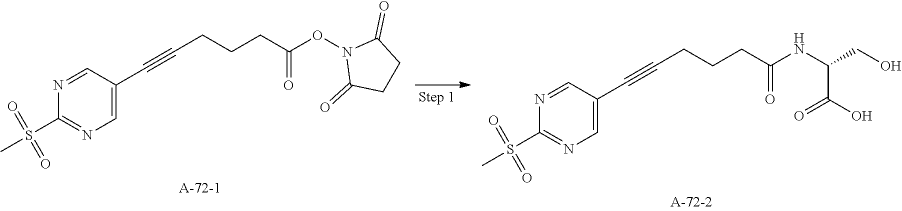 [SMILES: CS(=O)(=O)c1ncc(C#CCCCC(=O)ON2C(=O)CCC2=O)cn1 ; CS(=O)(=O)c1ncc(C#CCCCC(=O)N[C@H](CO)C(=O)O)cn1]

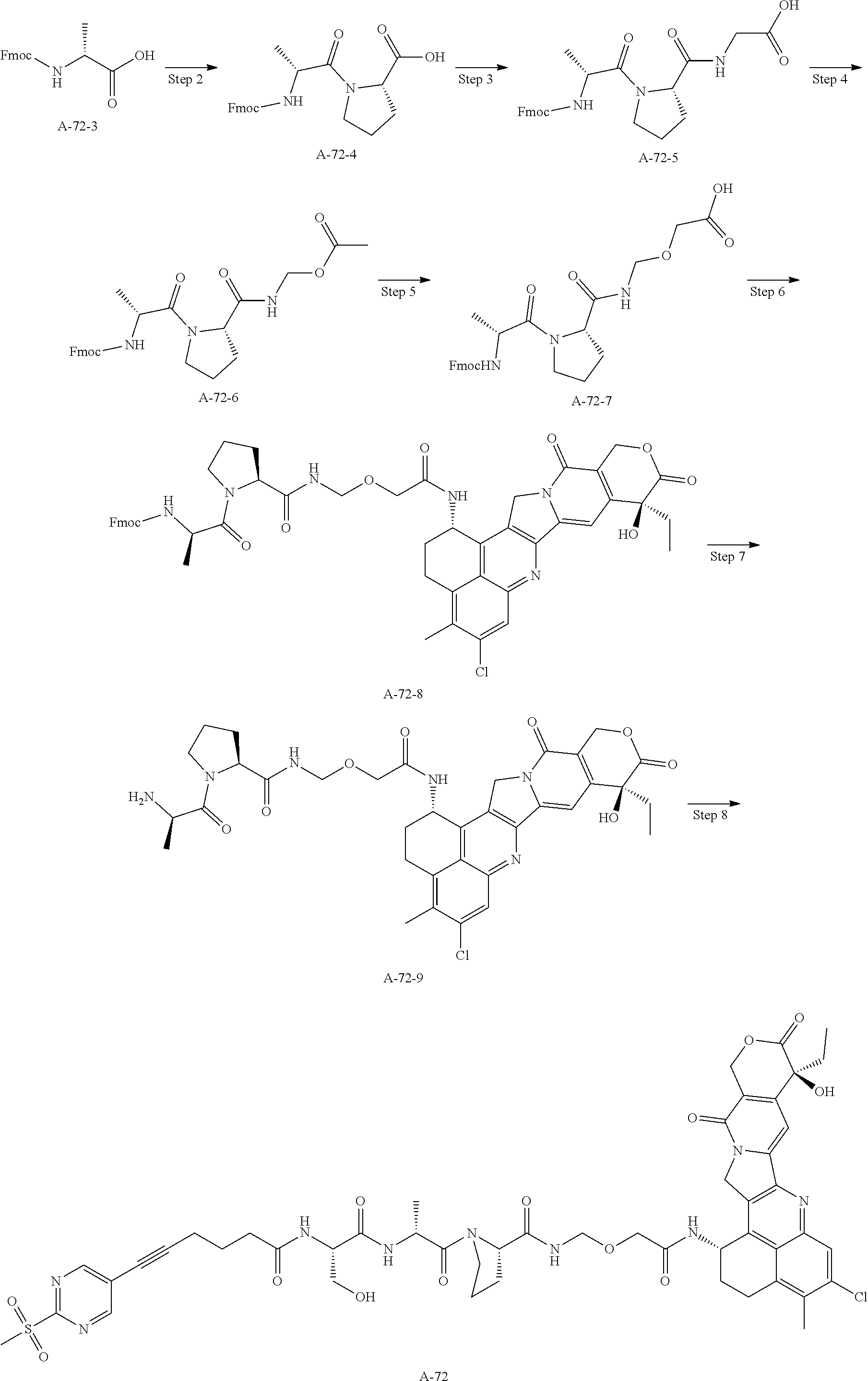

### Step 1: Preparation of (2S)-3-hydroxy-2-(6-(2-methylsulfonylpyrimidin-5-yl) hex-5-ynylamino)propionic acid (A-72-2)

(2,5-Dioxopyrrolidin-1-yl) 6-(2-methylsulfonylpyrimidin-5-yl)hex-5-ynoate (200.0 mg, 0.547 mmol), L-serine (63.3 mg, 0.602 mmol) and DIPEA (106.1 mg, 0.821 mmol) were added to DMF (4 mL) in sequence, reacted at 25° C. for 16 hours, and the reaction solution was directly purified by C18 reverse phase column (H₂O/ACN=20 to 50%, 0.1% FA) and lyophilized to obtain compound 2 (110 mg, 294.06 mol).

Its structural characterization data are as follows:

MS m/z (ESI): 356.1 [M+H]⁺

### Step 2: Preparation of (((9H-fluoren-9-yl)methoxy)carbonyl)-D-alanyl-L-proline (A-72-4)

(2R)-2-(9H-Fluoren-9-ylmethoxycarbonylamino)propionic acid (2.00 g, 6.42 mmol), 1-hydroxypyrrolidin-2,5-dione (887.2 mg, 7.71 mmol), and DCC (1.59 g, 7.71 mmol) were added to DMF (30 mL) in sequence, and reacted at 25° C. for 2 hours; L-proline (738.6 mg, 6.42 mmol) was added at once; DIPEA (829.1 mg, 6.42 mmol) was added dropwise; and the reaction was carried out at 25° C. for 4 hours. The reaction solution was poured into water (90 mL), and the insoluble solids were filtered out. The impurities in the filtrate were extracted with ethyl acetate (50 ml+30 ml). The aqueous phase was adjusted to pH 3-4 with hydrochloric acid, and then extracted with ethyl acetate twice (100 ml+50 ml). The organic phases were combined and washed with saturated sodium chloride aqueous solution, dried over anhydrous sodium sulfate, filtered and concentrated to obtain the title compound as crude product (2.40 g, 4.70 mmol), which was used directly in the next step without purification.

Its structural characterization data are as follows:

MS m/z (ESI): 409.2 [M+H]⁺

### Step 3: Preparation of (((9H-fluoren-9-yl)methoxy)carbonyl)-D-alanyl-L-alanylglycine (A-72-5)

(((9H-Fluoren-9-yl)methoxy)carbonyl)-D-alanyl-L-proline (1.60 g, 3.92 mmol), 1-hydroxypyrrolidin-2,5-dione (541.00 mg, 4.70 mmol), and DCC (969.90 mg, 4.70 mmol) were added to DMF (24 mL), and reacted at 25° C. for 2 hours; glycine (352.8 mg, 4.70 mmol) was added; DIPEA (506.20 mg, 3.92 mmol) was added dropwise; and the system was reacted at 25° C. for 4 hours. The reaction solution was poured into water (80 mL), the insoluble solid was filtered out, and the impurities were extracted with ethyl acetate (80 mL). The aqueous phase was adjusted to pH 3 with hydrochloric acid, extracted with ethyl acetate (100 mL), dried over anhydrous sodium sulfate, filtered and concentrated to obtain a crude product, which was purified by silica gel column (MeOH/DCM=5 to 15%) to obtain the title compound (560 mg, 1.08 mmol).

Its structural characterization data are as follows:

MS m/z (ESI): 466.2 [M+H]⁺

### Step 4: Preparation of methyl ((S)-1-((((9H-fluoren-9-yl)methoxy)carbonyl)-D-propylamino) pyrrolidine-2-carboxamido)acetate (A-72-6)

(((9H-Fluoren-9-yl)methoxy)carbonyl)-D-alanyl-L-alanylglycine (560 mg, 1.20 mmol), and Pb(OAc)₄ (1.17 g, 3.61 mmol) were dissolved in a mixed solvent of THF (15 mL) and toluene (5 mL), added with pyridine (9.52 mg, 0.120 mmol), heated to 70° C., refluxed for 4 hours, and filtered with diatomaceous earth. The filtrate was concentrated to obtain a crude product, which was purified by silica gel column (MeOH/DCM=5 to 10%) to obtain the title compound (530 mg, 750.74 μmol).

Its structural characterization data are as follows:

MS m/z (ESI): 502.2 [M+Na]⁺

### Step 5: Preparation of 2-(((S)-1-((((9H-fluoren-9-yl)methoxy)carbonyl)-D-propylamino) pyrrolidine-2-carboxamido)methyl)acetic acid (A-72-7)

Compound A-72-6 (480 mg, 1.00 mmol) and glycolic acid (228.38 mg, 3.00 mmol) were dissolved in THF (10 mL), added with p-toluenesulfonic acid (34.5 mg, 0.200 mmol), and the system was reacted at 25° C. for 4 hours. The reaction solution was concentrated to obtain a crude product, which was purified by column chromatography (MeOH/DCM=5 to 10%) and concentrated again to obtain the title compound (485 mg, 783.01 μmol).

Its structural characterization data are as follows:

MS m/z (ESI): 496.2 [M+H]⁺

### Step 6: Preparation of (9H-fluoren-9-yl)methyl ((R)-1-((S)-2-((2-(((2-(((1S,9S)-5-chloro-9-ethyl-9-hydroxy-4-methyl-10,13-dioxo-2,3,9,13,15-hexahydro-1H,12H-benzo[de]pyrano[3′,4′:6,7]indolizino[1,2-b]quinolin-1-yl)amino)-2-oxoethyl)methyl)carbamoyl)pyrrolidin-1-yl)-1-oxopropan-2-yl)carbamate (A-72-8)

(1S,9S)-1-Amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (100 mg, 0.182 mmol), 2-(((S)-1-((((9H-fluoren-9-yl)methoxy)carbonyl)-D-propylamino)pyrrolidine-2-carboxamido)methyl)acetic acid (135.6 mg, 0.218 mmol) were added to DMF (2 mL), added with DMTMM (100.99 mg, 0.365 mmol), and then added dropwise with DIPEA (70.8 mg, 0.547 mmol). The reaction system was reacted at 25° C. for 4 hours. The reaction solution was added with water (6 mL), extracted with ethyl acetate (20 mL), and concentrated to obtain a crude product. The crude product was purified by preparative high performance liquid chromatography (MeOH/DCM=5%) to obtain the title compound (37 mg, 39.81 μmol).

Its structural characterization data are as follows:

MS m/z (ESI): 929.2 [M+H]⁺

### Step 7: Preparation of (S)-1-(D-alanyl)-N-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)pyrrolidine-2-carboxamide (A-72-9)

Under nitrogen protection, (9H-fluoren-9-yl)methyl ((R)-1-((S)-2-((2-(((2-(((1S,9S)-5-chloro-9-ethyl-9-hydroxy-4-methyl-10,13-dioxo-2,3,9,13,15-hexahydro-1H,12H-benzo[de]pyrano[3′,4′:6,7]indolizino[1,2-b]quinolin-1-yl)amino)-2-oxoethoxy)methyl)carbamoyl)pyrrolidin-1-yl)-1-oxopropan-2-yl)carbamate (37.0 mg, 0.040 mmol) was dissolved in DMF (1 mL), and added with diethylamine (14.6 mg, 0.199 mmol). The reaction system was reacted at 25° C. for 2 hours. The reaction solution was directly purified by preparative high performance liquid chromatography and freeze-dried to obtain the title compound (11.0 mg, 13.87 μmol).

Its structural characterization data are as follows:

MS m/z (ESI): 707.3 [M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 28 |
| 2.00 | 10 | 90 | 28 |
| 18.00 | 80 | 20 | 28 |

### Step 8: Preparation of (S)—N-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)-1-((6-(2-(methylsulfonyl)pyrimidin-5-yl)hex-5-ynyl)-L-serine-D-propylamino)pyrrolidine-2-carboxamide (A-72)

(S)-1-(D-Alanyl)-N-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)pyrrolidine-2-carboxamide (11.0 mg, 0.0156 mmol) and (2S)-3-hydroxy-2-(6-(2-methylsulfonylpyrimidin-5-yl)hex-5-ynylamino)propionic acid (6.63 mg, 0.0187 mmol) were dissolved in DMF (0.5 mL), and added with HATU (11.8 mg, 0.0310 mmol) and DIPEA (6.00 mg, 0.0464 mmol), and the reaction system was reacted at 25° C. for 2 hours. The reaction solution was directly purified by preparative high performance liquid chromatography and freeze-dried to obtain the title compound (6.20 mg, 5.64 μmol).

Its structural characterization data are as follows:

MS m/z (ESI): 1044.3 [M+H]⁺

Its separation and purification methods are as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 30 |
| 2.00 | 20 | 80 | 30 |
| 18.00 | 90 | 10 | 30 |

### Example 14: (S)—N-(2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)-1-((4-((6-(2-(methylsulfonyl)pyrimidin-5-yl)hex-5-ynamido)methyl)benzoyl)-D-alanyl)pyrrolidine-2-carboxamide (A-74)

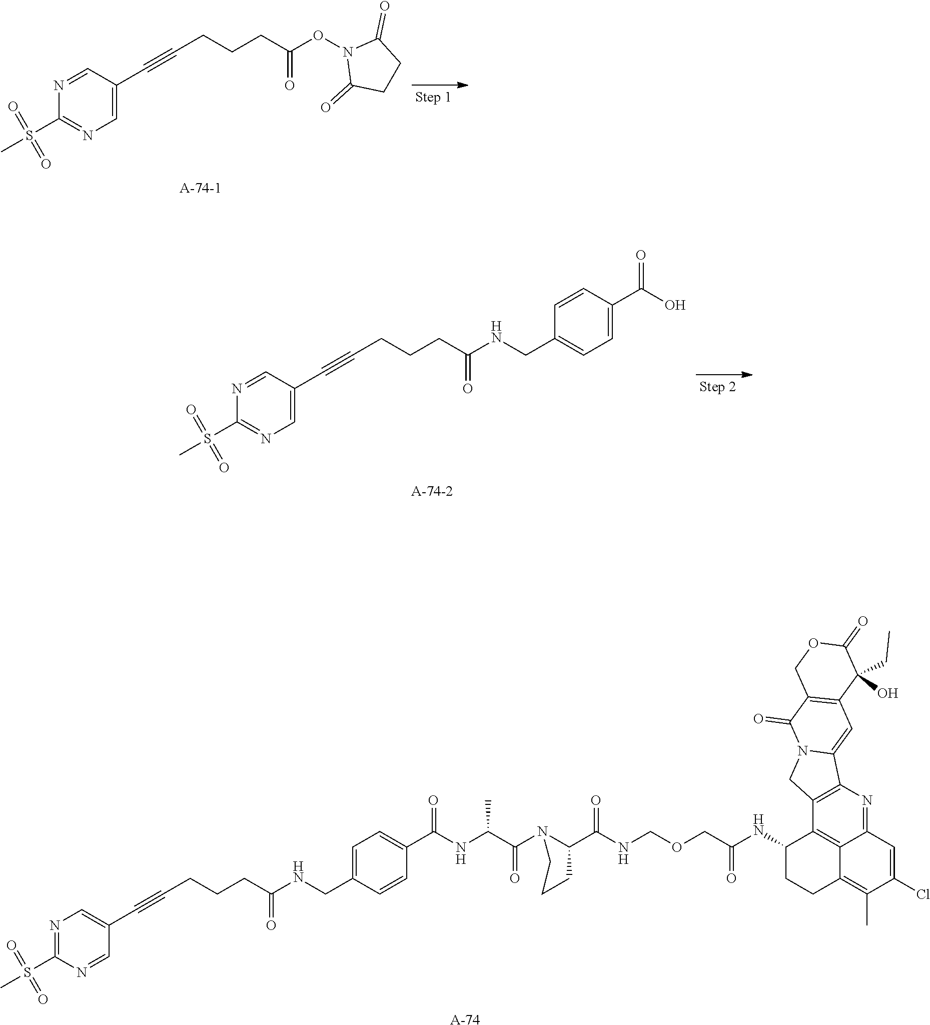

### Step 1: Preparation of 4-((6-(2-(methylsulfonyl)pyrimidin-5-yl)hex-5-ynamido) methyl)benzoic acid (A-74-2)

(2,5-Dioxopyrrolidin-1-yl) 6-(2-methylsulfonylpyrimidin-5-yl)hex-5-ynoate (200.0 mg, 0.547 mmol) and 4-(aminomethyl)benzoic acid (86.9 mg, 0.575 mmol) were added to DMF (4 mL), heated to 30° C. and reacted for 16 hours. The reaction solution was poured into water (15 mL) and stirred for 1 hour. The solid precipitated and was filtered. The filter cake was washed with water (5 mL) and methyl tert-butyl ether (5 mL) in turn, and then dried to obtain the title compound (169 mg, 399.94 μmol).

Its structural characterization data are as follows:

MS m/z (ESI): 402.2 [M+H]⁺

### Step 2: Preparation of (S)—N-(2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)-1-((4-((6-(2-(methylsulfonyl)pyrimidin-5-yl)hex-5-ynamido)methyl)benzoyl)-D-alanyl)pyrrolidine-2-carboxamide (A-74)

Under nitrogen protection, (S)-1-(D-alanyl)-N-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)pyrrolidine-2-carboxamide (16.0 mg, 0.0195 mmol), 4-((6-(2-(methylsulfonyl)pyrimidin-5-yl)hex-5-ynamido)methyl)benzoic acid (9.4 mg, 0.0238 mmol) were added to DMF (0.5 mL), and then added with HATU (14.8 mg, 0.039 mmol) and DIPEA (7.6 mg, 0.058 mmol). The reaction system was reacted at 25° C. for 2 hours. The reaction solution was directly purified by preparative high performance liquid chromatography and freeze-dried to obtain the title compound (6.00 mg, 5.34 μmol).

Its structural characterization data are as follows:

MS m/z (ESI): 1090.3 [M+H]⁺

Its separation and purification method was as follows:

Chromatographic column: Waters SunFire Prep C18
OBD (5 μm*19 mm× 250 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 22 |
| 2.00 | 30 | 70 | 22 |
| 18.00 | 90 | 10 | 22 |

### Example 15: N-((10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-methyl-1,6,9,12-tetraoxo-3-oxa-5,8,11-triazatetradecane-13-yl)-6-(2-(methylsulfonyl)pyrimidin-5-yl)hex-5-ynamide (A-76)

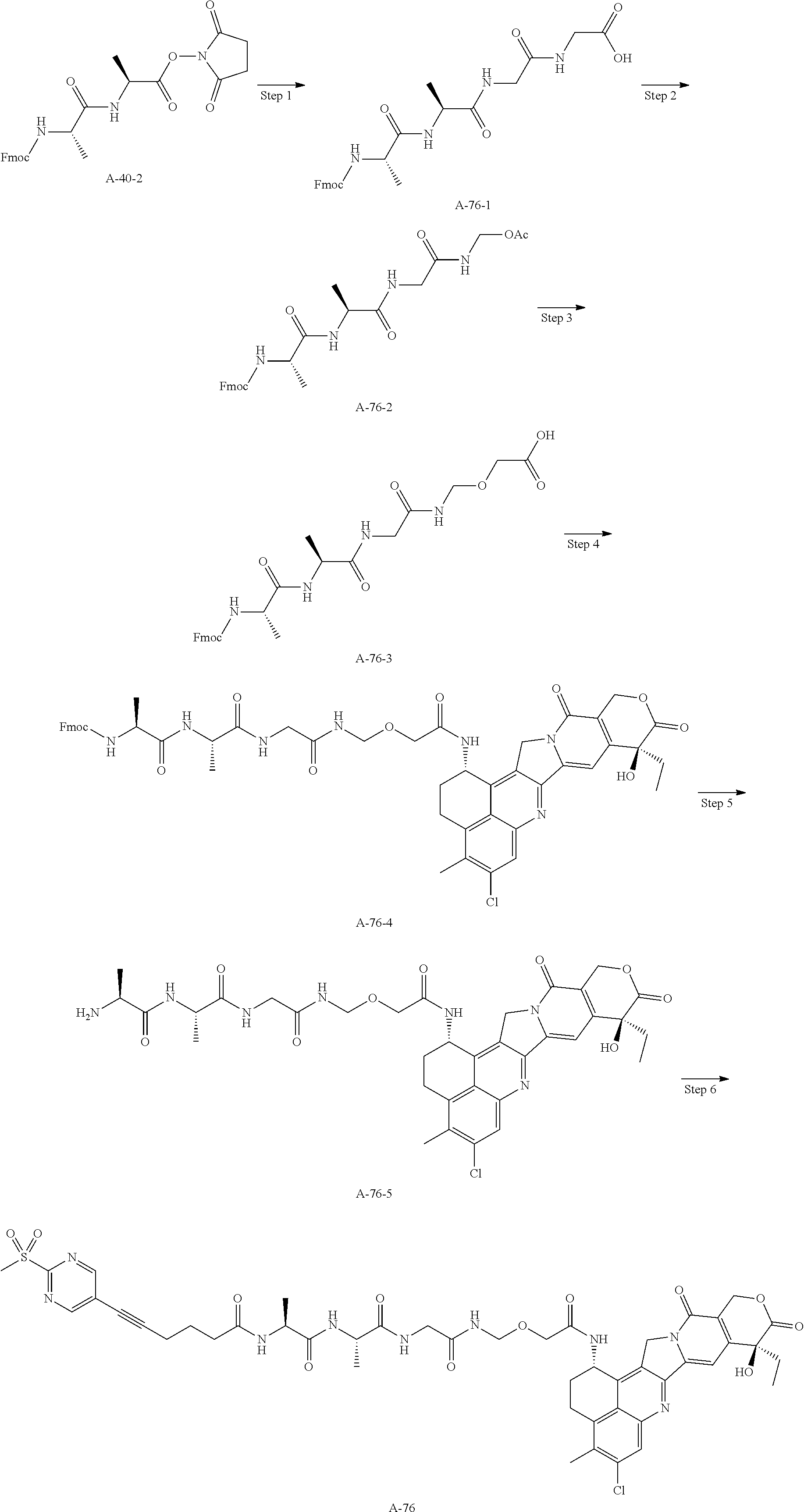

### Step 1: Preparation of ((9H-fluoren-9-yl)methoxy)carbonyl)-L-alanine-L-alanine-glycylglycine (A-76-1)

2,5-Dioxopyrrolidin-1-yl ((9H-fluoren-9-yl)methoxy)carbonyl)-L-alanine-L-alaninate (1.0 g, 2.09 mmol) and glycylglycine (551.09 mg, 4.17 mmol) were dissolved in acetone (12 mL) and water (6 mL), then added with NaHCO₃ (350.41 mg, 4.17 mmol), and stirred at room temperature for 2 hours. The reaction solution was added with water (20 ml), adjusted to pH 3-4 with concentrated hydrochloric acid, and extracted with ethyl acetate 3 times (15 mL×3). The organic phases were combined and washed with saturated sodium chloride aqueous solution (15 mL), dried over anhydrous sodium sulfate, filtered and concentrated to obtain 3.4 g of the title compound as a crude product, which was used directly in the next step without purification.

Its structural characterization data are as follows:

MS m/z (ESI): 497.2 [M+H]⁺, 515.2 [M+H+H₂O]⁺

### Step 2: Preparation of (5S,8S)-1-(9H-fluoren-9-yl)-5,8-dimethyl-3,6,9,12-tetraoxo-2-oxa-4,7,10,13-tetraazatetradecan-14-yl acetate (A-76-2)

((9H-Fluoren-9-yl)methoxy)carbonyl)-L-alanine-L-alanine-glycylglycine (720 mg, 1.45 mmol) was dissolved in THF (10 mL), and added with pyridine (344.11 mg, 4.35 mmol) and lead tetraacetate (1.93 g, 4.35 mmol). The system turned yellow, subjected to nitrogen replacement, and heated to 70° C. for 4 hours. The reaction solution was added with water (20 mL), and extracted with ethyl acetate 3 times (10 mL×3). The organic phase was washed with saturated ammonium chloride aqueous solution (10 mL) and saturated sodium chloride aqueous solution (10 mL) in sequence, dried over anhydrous sodium sulfate, filtered and concentrated, and the residue was purified by silica gel column chromatography (ethyl acetate/petroleum ether=50% to 100%) to obtain the title compound (270 mg, 528.8 μmol).

Its structural characterization data are as follows:

MS m/z (ESI): 528.2 [M+H+H₂O]⁺

### Step 3: Preparation of (5S,8S)-1-(9H-fluoren-9-yl)-5,8-dimethyl-3,6,9,12-tetraoxo-2,15-dioxa-4,7,10,13-tetraazaheptadecan-17-oic acid (A-76-3)

(5S,8S)-1-(9H-Fluoren-9-yl)-5,8-dimethyl-3,6,9,12-tetraoxo-2-oxo-4,7,10,13-tetraazatetradecan-14-yl acetate (80 mg, 156.70 mol) was suspended in THF (2 mL), added with glycolic acid (59.59 mg, 783.49 μmol), then added with TsOH (2.70 mg, 15.67 μmol), and stirred at room temperature for 6 hours. The insoluble matter gradually dissolved. After the reaction was completed, the reaction solution was concentrated under reduced pressure to remove the solvent, and the resultant concentrate was purified by preparative high performance liquid chromatography and freeze-dried to obtain 45 mg of the title compound.

Its structural characterization data are as follows:

MS m/z (ESI): 535.3 [M+Na]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 28 |
| 2.00 | 30 | 70 | 28 |
| 20.00 | 70 | 30 | 28 |

### Step 4: Preparation of ((9H-fluoren-9-yl)methyl ((10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-methyl-1,6,9,12-tetraoxo-5,8,11-triazatetradecan-13-yl)carbamate (A-76-4)

(1S,9S)-1-Amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (26.02 mg, 47.48 μmol) and (5S,8S)-1-(9H-fluoren-9-yl)-5,8-dimethyl-3,6,9,12-tetraoxo-2,15-dioxa-4,7,10,13-tetraazaheptadecan-17-oic acid (25.00 mg, 47.48 μmol) were dissolved in DMF (1.5 mL), added with DIPEA (12.27 mg, 94.96 μmol) and HATU (21.66 mg, 56.98 μmol), and stirred at room temperature for 0.5 hours. After the reaction was completed, the reaction solution was directly used in the next reaction without treatment.

Its structural characterization data are as follows:

MS m/z (ESI): 960.3[M+H]⁺

### Step 5: Preparation of ((S)-2-amino-N—((S)-1-((2-((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)2-oxoethyl)amino)-1-oxopropan-2-yl)propanamide (A-76-5)

The reaction solution containing (9H-fluoren-9-yl)methyl ((10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-methyl-1,6,9,12-tetraoxo-5,8,11-triazatetradecan-13-yl)carbamate (45 mg, 46.85 μmol) in the previous step was added with DMF (1 mL) and diethylamine (0.2 mL), and stirred at room temperature for 1 hour. After the reaction was completed, the reaction solution was directly evaporated under reduced pressure to remove diethylamine, and the residue was directly used in the next reaction.

Its structural characterization data are as follows:

MS m/z (ESI): 757.3 [M+H+H₂O]⁺

### Step 6: Preparation of (N-((10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-methyl-1,6,9,12-tetraoxo-3-oxa-5,8,11-triazatetradecan-13-yl)-6-(2-(methylsulfonyl)pyrimidin-5-yl)hex-5-ynamide (A-76)

((S)-2-Amino-N—((S)-1-((2-((2-)(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)2-oxoethyl)amino)-1-oxopropan-2-yl)propanamide (34 mg, 46.06 μmol) was dissolved in DMF (1 mL), added with DIPEA (5.95 mg, 46.06 μmol), then added with 2,5-dioxopyrrolidin-1-yl 6-(2-(methylsulfonyl)pyrimidin-5-yl)hex-5-ynoate (16.83 mg, 46.06 μmol), and stirred at room temperature for 1 hour. After the reaction was completed, the residue was directly purified by preparative high performance liquid chromatography and freeze-dried to obtain the title compound (17.24 mg, 16.35 μmol).

Its structural characterization data are as follows:

MS m/z (ESI): 988.4 [M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 25 | 75 | 28 |
| 2.00 | 25 | 75 | 28 |
| 20.00 | 90 | 10 | 28 |

### Example 16: Preparation of N-((7S,10S)-1-(((1S,9S)-5-chloro-9-ethyl-9-hydroxy-4-methyl-10,13-dioxo-2,3,9,10,15-hexahydro-1H,12H-benzo[de]pyrano[3′,4′:6,7]indolizino[1,2-b]quinolin-1-yl)amino)-10-isopropyl-7-methyl-1,6,9,12,15-pentaoxo-3-oxa-5,8,11,14-tetraazahexadecan-16-yl)-6-(2-(methylsulfonyl)pyrimidin-5-yl)hex-5-ynamide (A-70)

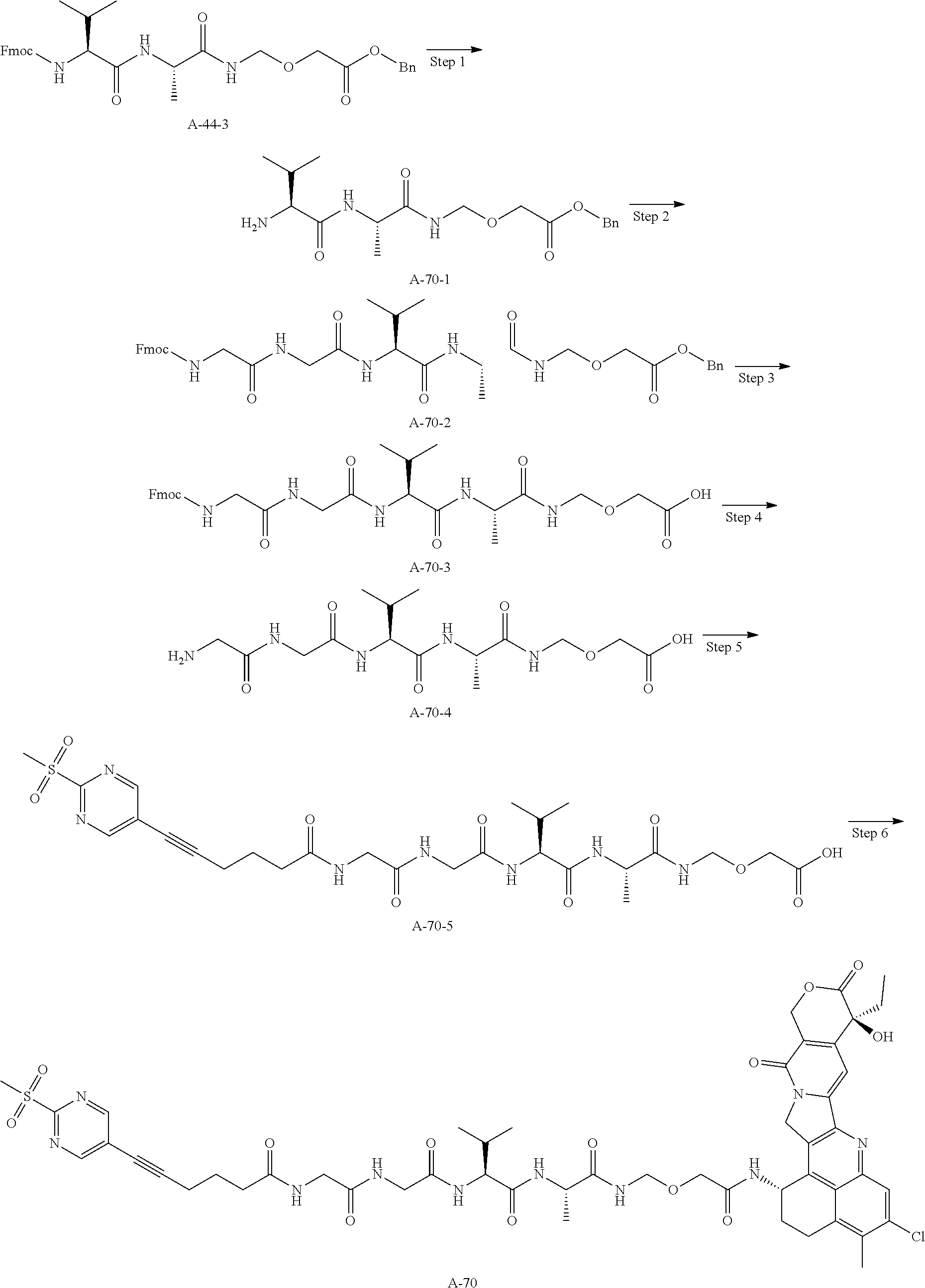

### Step 1: Preparation of benzyl 2-(((S)-2-((S(-2-amino-3-methylbutyramido)propionamido) methoxy)acetate (A-70-1)

Benzyl (5S,8S)-1-(9H-fluoren-9-yl)-5-isopropyl-8-methyl-3,6,9-trioxo-2,12-dioxa-4,7,10-triazatetradecan-1-oate (2 g, 3.40 mmol) was dissolved in DMF (20 mL), added with DBU (414 mg, 2.72 mmol, 410 μL), and stirred at 25° C. for 1 hour. The reaction solution was used directly in the next step without purification.

### Step 2: Preparation of benzyl (11S,14S)-1-(9H-fluoren-9-yl)-11-isopropyl-14-methyl-3,6,9,12,15-pentaoxo-2,18-dioxa-4,7,10,13,16-pentaazaeicosanoate (A-70-2)

To the reaction solution of the previous step, (((9H-fluoren-9-yl)methoxy)carbonyl)glycylglycine (1.16 g, 3.28 mmol), EDCI (755 mg, 3.94 mmol) and HOBt (532 mg, 3.94 mmol) were added in sequence. After the addition was completed, the mixture was stirred at 25° C. for 2 hours. The reaction solution was added with water (100 mL), and solid precipitated and was filtered. The filter cake was slurried (petroleum ether/ethyl acetate=3/1, 40 mL×2) and filtered again, and dried to obtain the title compound (2.2 g, 3.13 mmol).

Its structural characterization data are as follows:

MS m/z (ESI): 719.4 [M+H]⁺

### Step 3: Preparation of (11S,14S)-1-(9H-fluoren-9-yl)-11-isopropyl-14-methyl-3,6,9,12,15-pentaoxo-2,18-dioxa-4,7,10,13,16-pentaazaeicosanoic acid (A-70-3)

Benzyl (11S,14S)-1-(9H-fluoren-9-yl)-11-isopropyl-14-methyl-3,6,9,12,15-pentaoxo-2,18-dioxa-4,7,10,13,16-pentaazaeicosanoate (2 g, 2.85 mmol) and 10% Pd/C (0.5 g) were added to DMF (30 mL), and subjected to hydrogen replacement 3 times (15 PSI). The reaction solution was filtered, then added with water (30 mL), and filtered to obtain a crude product, which was slurried (petroleum ether/ethyl acetate=3/1, 40 mL×2), filtered again, and dried to obtain the title compound (1.03 g, 1.51 mmol).

Its structural characterization data are as follows:

MS m/z (ESI): 568.2 [M+H]⁺

### Step 4: Preparation of (7S,10S)-16-amino-10-isopropyl-7-methyl-6,9,12,15-tetraoxo-5,8,11,14-tetraazahexadecanoic acid (A-70-4)

(11S,14S)-1-(9H-Fluoren-9-yl)-11-isopropyl-14-methyl-3,6,9,12,15-pentaoxo-2,18-dioxa-4,7,10,13,16-pentaazaeicosenoic acid (30 mg, 49.05 μmol) and diethylamine (0.5 mL) were dissolved in DMF (2.5 mL), and stirred at room temperature for 1 hour. The title compound as crude product was obtained by concentration under reduced pressure and used directly in the next step without purification.

### Step 5: Preparation of (7S,10S)-10-isopropyl-7-methyl-23-(2-methylsulfonyl)pyrimidin-5-yl)-6,9,12,15,18-pentaoxo-3-oxa-5,8,11,14,17-pentaazadocos-22-ynoic acid (A-70-5)

(2,5-Dioxopyrrolidin-1-yl) 6-(2-methylsulfonylpyrimidin-5-yl)hex-5-ynoate (17.92 mg, 49.05 μmol) was added to the reaction system in the previous step, and stirred at 25° C. for 1 hour. Methyl tert-butyl ether (30 mL) was added to the reaction solution, and a large amount of solid precipitated, and was filtered to obtain a crude product (20 mg), which was used directly in the next step without purification.

### Step 6: Preparation of N-(7S,10S)-1-((1S,9S)-5-chloro-9-ethyl-9-hydroxy-4-methyl-10,13-dioxo-2,3,9,10,13,15-hexahydro-1H,12H-benzofuran[3′,4′:6,7]indolizino[1,2-b]quinolin-1-yl)amino)-10-isopropyl-7-methyl-1,6,9,12,15-pentaoxo-3-oxa-5,8,11,14-tetraazahexadecan-16-yl)-6-(2-(methylsulfonyl)pyrimidin-5-yl)hexan-5-imide (A-70)

(7S,10S)-10-Isopropyl-7-methyl-23-(2-methylsulfonyl)pyrimidin-5-yl)-6,9,12,15,18-pentaoxa-3-oxa-5,8,11,14,17-pentaazadocos-22-ynoic acid (20 mg, crude) and (1S,9S)-1-amino-5-chloro-9-ethyl-9-hydroxy-4-methyl-1,2,3,9,12,15-hexahydro-10H,13H-benzo[de]pyrano[3′,4′:6,7]indolizino[1,2-b]quinolin-10,13-dione (17.13 mg, 31.27 μmol) were dissolved in DMF (1 mL), then added with HATU (14.26 mg, 37.52 μmol) and N,N-diisopropylethylamine (12.12 mg, 93.80 μmol), and the reaction system was stirred at room temperature for 1 hour. The reaction solution was purified by preparative high performance liquid chromatography and freeze-dried to obtain the title compound (8.45 mg, 7.79 μmol).

Its structural characterization data are as follows:

MS m/z (ESI): 1074.3 [M+H]⁺

Its preparation method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 28 |
| 2.00 | 30 | 70 | 28 |
| 18.00 | 90 | 10 | 28 |

### Example 17: Preparation of N-((3S,8S,11S,14S)-1-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-3,8,11-trimethyl-1,7,10,13-tetraoxo-4-oxa-6,9,12-triazapentadecan-14-yl)-6-(2-(methylsulfonyl)pyrimidin-5-yl)hex-5-ynamide (A-36)

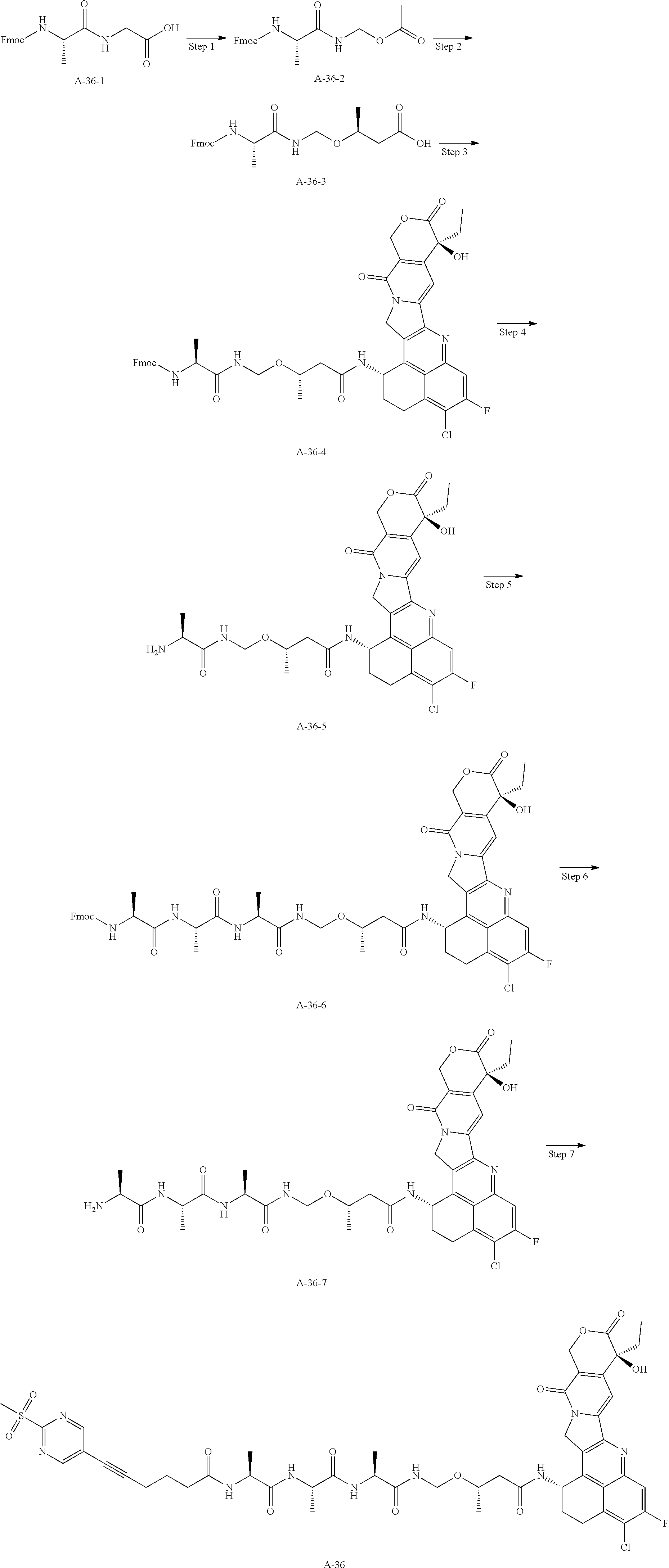

### Step 1: Preparation of methyl (S)-(2-((((9H-fluoren-9-yl)methoxy)carbonyl)amino) propionamido)acetate (A-36-2)

(((9H-Fluoren-9-yl)methoxy)carbonyl)-L-alanine-glycine (10.00 g, 27.15 mmol) and lead tetraacetate (26.49 g, 81.44 mmol) were dissolved in a mixed solvent of tetrahydrofuran (150 mL) and toluene (50 mL), added with pyridine (214.7 mg, 2.71 mmol), heated to 70° C. and reacted under refluxing for 4 hours. The reaction solution was filtered with diatomaceous earth, and the filtrate was concentrated to obtain about 20 ml of residue, in which a small amount of solid precipitated. Methyl tert-butyl ether (150 ml) was added, stirred for 1 hour, and filtered. The filter cake was collected, and dried to obtain the title compound (7.90 g, 18.59 mmol).

Its structural characterization data are as follows:

MS m/z (ESI): 405.2 [M+Na]⁺

### Step 2: Preparation of (5S,10S)-1-(9H-fluoren-9-yl)-5,10-dimethyl-3,6-dioxo-2,9-dioxa-4,7-diazadodecan-12-oic acid (A-36-3)

Methyl (S)-(2-((((9H-fluoren-9-yl)methoxy)carbonyl)amino)propionamido)acetate (500 mg, 1.31 mmol) and S-3-hydroxybutyric acid (204.2 mg, 1.96 mmol) were dissolved in tetrahydrofuran (10 mL), added with p-toluenesulfonic acid (45.0 mg, 0.261 mmol), heated to 30° C. and reacted for 6 hours. The reaction solution was concentrated to obtain a crude product and purified by column chromatography (MeOH/DCM=5 to 10%) to obtain the title compound (260 mg, 0.488 mmol).

Its structural characterization data are as follows:

MS m/z (ESI): 449.2 [M+Na]⁺

### Step 3: Preparation of (9H-fluoren-9-yl)methyl ((S)-1-(S)-4-(((1S,9S)-4-chloro-9-ethyl-5-fluoro-9-hydroxy-10,13-dioxo-2,3,9,13,15-hexahydro-1H,12H-benzo[de]pyrano[3′,4′:6,7]indolizino[1,2-b]quinolin-1-yl)amino)-4-oxobutan-2-yl)oxy)methyl)amino)-1-oxopropan-2-yl)carbamate (A-36-4)

(1S,9S)-1-Amino-4-chloro-9-ethyl-5-fluoro-9-hydroxy-1,2,3,9,12,15-hexahydro-10H,13H-benzo[de]pyrano[3′,4′:6,7]indolizino[1,2-b]quinolin-10,13-dione (100.0 mg, 0.175 mmol, trifluoroacetate) and (5S,10S)-1-(9H-fluoren-9-yl)-5,10-dimethyl-3,6-dioxo-2,9-dioxa-4,7-diazadodecan-12-oic acid (89.8 mg, 0.211 mmol) were dissolved in DMF (3 mL), added with HATU (138.8 mg, 0.365 mmol) and DIPEA (68.0 mg, 0.526 mmol), and reacted at 25° C. for 1 hour. The system was added with water (10 ml), extracted with ethyl acetate (30 ml), and concentrated to obtain a crude product, which was purified by thin layer chromatography (MeOH/DCM=5%) to obtain the title compound (40.0 mg, 0.037 mmol).

Its structural characterization data are as follows:

MS m/z (ESI): 864.4 [M+H]⁺

### Step 4: Preparation of (S)-3-(((S)-2-aminopropionamido)methoxy)-N-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)butanamide (A-36-5)

(9H-Fluoren-9-yl)methyl ((S)-1-(S)-4-(((1S,9S)-4-chloro-9-ethyl-5-fluoro-9-hydroxy-10,13-dioxo-2,3,9,13,15-hexahydro-1H,12H-benzo[de]pyrano[3′,4′:6,7]indolizino[1,2-b]quinolin-1-yl)amino)-4-oxobutan-2-yl)oxy)methyl)amino)-1-oxopropan-2-yl)carbamate (40.00 mg, 0.046 mmol) was dissolved in DMF (1 mL), added with DIPEA (16.9 mg, 0.231 mmol), and reacted at 25° C. for 1 hour. The reaction solution was directly purified by preparative high performance liquid chromatography and freeze-dried to obtain the title compound (11.0 mg, 0.017 mmol).

Its structural characterization data are as follows:

MS m/z (ESI): 642.2 [M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
ammonium bicarbonate aqueous solution)

| | | | Flow rate |
|---|---|---|---|
| Time [min] | Mobile phase A [%] | Mobile phase B [%] | [mL/min] |
| 0.00 | 20 | 80 | 28 |
| 2.00 | 20 | 80 | 28 |
| 24.00 | 80 | 20 | 28 |

### Step 5: Preparation of (9H-fluoren-9-yl)methyl ((3S,8S,11S,14S)-1-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-3,8,11-trimethyl-1,7,10,13-tetraoxo-4-oxa-6,9,12-triazapentadecan-14-yl)carbamate (A-36-6)

(S)-3-(((S)-2-Aminopropionamido)methoxy)-N-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)butanamide (11 mg, 0.017 nmol) and (((9H-fluoren-9-yl)methoxy)carbonyl)-L-alanine-L-alanine (7.9 mg, 0.20 mmol) were dissolved in DMF (1 mL), added with HATU (13.0 mg, 0.034 mmol) and DIPEA (6.6 mg, 0.051 mmol), and reacted at 25° C. for 1 hour. The reaction solution was purified by preparative high performance liquid chromatography and freeze-dried to obtain the title compound (13.0 mg, 0.012 mmol).

Its structural characterization data are as follows:

MS m/z (ESI): 1006.4 [M+H]⁺

Its preparation method was as follows:

Chromatographic column: Waters SunFire Prep C18
OBD (5 μm*19 mm*150 mm)
Mobile phase A: acetonitrile; Mobile phase B: water (0.05%
FA aqueous solution)

| | | | Flow rate |
|---|---|---|---|
| Time [min] | Mobile phase A [%] | Mobile phase B [%] | [mL/min] |
| 0.00 | 40 | 60 | 30 |
| 2.00 | 40 | 60 | 30 |
| 18.00 | 90 | 10 | 30 |

### Step 6: Preparation of (S)-3-(((S)-2-((S-2-aminopropionamide)propionamido)methoxy)-N-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)butanamide (A-36-7)

(9H-Fluoren-9-yl)methyl ((3S,8S,11S,14S)-1-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-3,8,11-trimethyl-1,7,10,13-tetraoxo-4-oxa-6,9,12-triazapentadecan-14-yl)carbamate (13.0 mg, 0.012 mmol) was dissolved in DMF (0.5 mL), added with diethylamine (9.4 mg, 0.129 mmol), and reacted at 25° C. for 1 hour. The reaction solution was directly purified by high-performance liquid chromatography to obtain compound A-36-7 (6.0 mg, 0.0069 mmol, formate).

Its structural characterization data are as follows:

MS m/z (ESI): 784.3 [M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 2.00 | 10 | 90 | 30 |
| 18.00 | 70 | 30 | 30 |

### Step 7: Preparation of N-((3S,8S,11S,14S)-1-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-3,8-11-trimethyl-1,7,10,13-tetraoxo-4-oxa-6,9,12-triazapentadecan-14-yl)-6-(2-(methylsulfonyl)pyrimidin-5-yl)hex-5-ynamide (A-36)

(S)-3-(((S)-2-((S-2-Aminopropionamido)propionamido)methoxy)-N-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)butanamide (6.0 mg, 0.0069 mmol, formate) and 2,5-dioxopyrrolidin-1-yl 6-(2-(methylsulfonyl)pyrimidin-5-yl)hex-5-ynoate (4.0 mg, 0.011 mmol) were dissolved in DMF (0.5 mL), added with DIPEA (1.9 mg, 0.014 mmol), and reacted at 25° C. for 2 hours. The reaction solution was directly purified by preparative high performance liquid chromatography and freeze-dried to obtain the title compound (3.0 mg, 0.0028 mmol).

Its structural characterization data are as follows:

MS m/z (ESI): 1034.3 [M+H]⁺

The preparation method is as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 30 |
| 2.00 | 20 | 80 | 30 |
| 18.00 | 80 | 20 | 30 |

### Example 18: Preparation of N—(((S)-1-(((S)-1-(S)-4-(((1S,9S)-4-chloro-9-ethyl-5-fluoro-9 hydroxy-10,13-dioxo-2,3,9,10,13,15-hexahydro-1H,12H-benzo[de]pyrano[3′,4′:6,7]indolizino[1,2-b]quinolin-1-yl)amino)-4-oxobutan-2-yl(oxy)methyl)amino)-1-oxopropan-2-yl)amino 3-methyl-1-oxobutan-2-yl)-6-(2-(methylsulfonyl)pyrimidin-5-yl)hex-5-ynamide (A-46)

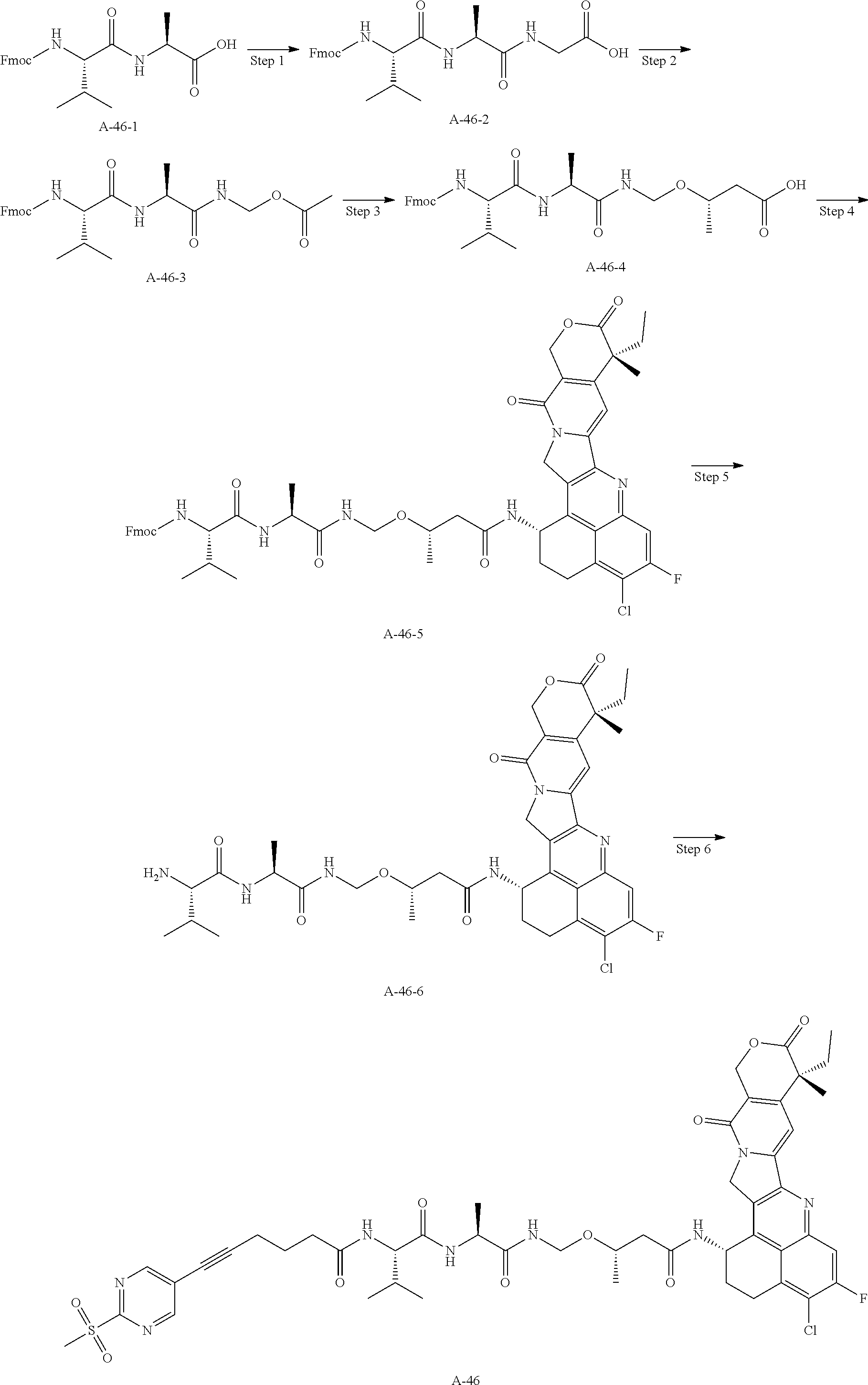

### Step 1: Preparation of (((9H-fluoren-9-yl)methoxy)carbonyl)-L-valine-L-alanine-glycine (A-46-2)

(((9H-Fluoren-9-yl)methoxy)carbonyl)-L-valine-L-alanine (1.00 g, 2.44 mmol), HOSU (336.5 mg, 2.92 mmol) and DCC (603.2 mg, 2.92 mmol) were added to DMF (15 mL), reacted at 25° C. for 16 hours, then added with glycine (201.7 mg, 2.69 mmol), added dropwise with DIPEA (315.8 mg, 2.44 mmol), and reacted at 25° C. for 5 hours. The reaction solution was poured into water (50 ml), the insoluble solid was filtered off, and the filtrate was extracted with ethyl acetate (50 ml+30 ml). The aqueous phase was adjusted to pH=3 with hydrochloric acid, and extracted with ethyl acetate (80 ml+50 ml). The organic phases were combined, washed with brine (10 ml), and concentrated to obtain the title compound (805 mg, 1.38 mmol).

Its structural characterization data are as follows:

MS m/z (ESI): 499.5 [M+Na]⁺

### Step 2: Preparation of (5S,8S)-1-(9H-fluoren-9-yl)-5-isopropyl-8-methyl-3,6,9-trioxo-2-oxa-4,7,10-triazaundecan-11-yl acetate (A-46-3)

(((9H-Fluoren-9-yl)methoxy)carbonyl)-L-valine-L-alanine-glycine (750 mg, 1.60 mmol) and lead tetraacetate (1.57 g, 4.81 mmol) were dissolved in a mixed solvent of tetrahydrofuran (30 mL) and toluene (10 mL), added with pyridine (25.4 mg, 0.32 mmol), heated to 70° C. and reacted under refluxing for 4 hours. The reaction solution was filtered through diatomaceous earth, and the filtrate was concentrated to obtain a crude product, which was purified by column chromatography (MeOH/DCM=5 to 10%) and concentrated again to obtain the title compound (230 mg, 0.043 mmol).

Its structural characterization data are as follows:

MS m/z (ESI): 504.2 [M+Na]⁺

### Step 3: Preparation of (5S,8S,13S)-1-(9H-fluoren-9-yl)-5-isopropyl-8,13-dimethyl-3,6,9-trioxo-2,12-dioxa-4,7,10-triazapentadecan-15-oic acid (A-46-4)

(5S,8S)-1-(9H-Fluoren-9-yl)-5-isopropyl-8-methyl-3,6,9-trioxo-2-oxa-4,7,10-triazaundecan-11-yl acetate (210.0 mg, 0.436 mmol) and (S)-3-hydroxybutyric acid (68.1 mg, 0.654 mmol) were dissolved in tetrahydrofuran (5 mL), added with p-toluenesulfonic acid (15.02 mg, 0.087 μmol), heated to 30° C. and reacted for 2 hours. The reaction solution was concentrated to dryness, added with methyl tert-butyl ether (10 ml), and stirred for 10 minutes, and solid precipitated. The filter cake after filtration was purified by C₁₈ reaction column (ACN/H₂O=30 to 70%) to obtain the title compound (85.0 mg, 0.097 mmol).

Its structural characterization data are as follows:

MS m/z (ESI): 548.3 [M+Na]⁺

### Step 4: Preparation of (9H-fluoren-9-yl)methyl ((S)-1-(S)-4-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolyl)amino)-4-oxobutan-2-yl)oxy)amino-1-oxopropan-2-yl)amino)-3-methyl-1-oxobutan-2-yl)carbamate (A-46-5)

(1S,9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinolin-10,13-dione (40.0 mg, 0.070 mmol, trifluoroacetate) and (5S,8S,13S)-1-(9H-fluoren-9-yl)-5-isopropyl-8,13-dimethyl-3,6,9-trioxo-2,12-dioxa-4,7,10-triazapentadecan-15-oic acid (44.3 mg, 0.084 mmol) were dissolved in DMF (3 mL), added with HATU (53.4 mg, 0.140 mmol) and DIPEA (27.2 mg, 0.210 mmol), and reacted at 25° C. for 1 hour. The product was added with water (10 ml) and ethyl acetate (30 ml) for extraction, and concentrated to obtain a crude product, which was purified by thin layer chromatography (MeOH/DCM=5%) to obtain the title compound (40.0 mg, 0.042 mmol).

Its structural characterization data are as follows:

MS m/z (ESI): 963.3 [M+H]⁺

### Step 5: Preparation of (S)-2-amino-N—((S)-1-(S)-4-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-4-oxobutan-2-yl)oxy)methyl)amino)-1-oxopropan-2-yl)-3-methylbutanamide (A-46-6)

(9H-Fluoren-9-yl)methyl ((S)-1-(S)-4-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolinyl)amino)-4-oxobutan-2-yl)oxy)amino-1-oxopropan-2-yl)amino)-3-methyl-1-oxobutan-2-yl)carbamate (40.0 mg, 0.042 mmol) was dissolved in DMF (1 mL), added with diethylamine (15.2 mg, 0.208 mmol), and reacted at 25° C. for 1 hour. The reaction solution was directly purified by preparative high performance liquid chromatography and freeze-dried to obtain the title compound (12.0 mg, 16.2 mmol, formate).

Its structural characterization data are as follows:

MS m/z (ESI): 741 [M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 2.00 | 10 | 90 | 30 |
| 18.00 | 80 | 20 | 30 |

### Step 6: Preparation of N—(((S)-1-(((S)-1-(S)-4-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-4-oxobutan-2-yl(oxy)methyl)amino)-1-oxopropan-2-yl)amino-3-methyl-1-oxobutan-2-yl)-6-(2-(methylsulfonyl)pyrimidin-5-yl)hex-5-ynamide (A-46)

(S)-2-Amino-N—((S)-1-(S)-4-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-4-oxobutan-2-yl)oxy)methyl)amino)-1-oxopropan-2-yl)-3-methylbutanamide (11.3 mg, 0.015 mmol) and (2,5-dioxopyrrolidin-1-yl) 6-(2-methylsulfonylpyrimidin-5-yl)hex-5-ynoate (8.4 mg, 0.023 mmol) were dissolved in DMF (0.5 mL), added with DIPEA (3.9 mg, 0.031 mmol), and reacted at 25° C. for 2 hours. The reaction solution was purified by preparative high performance liquid chromatography and freeze-dried to obtain the title compound (4.20 mg, 0.004 mmol).

Its structural characterization data are as follows:

MS m/z (ESI): 991.4 [M+H]⁺

Its separation and purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 30 |
| 2.00 | 30 | 70 | 30 |
| 18.00 | 90 | 10 | 30 |

### Example 19: N-((9S,12S,15S)-1-((S)-9-chloro-4-ethyl-8-fluoro-4-hydroxy-3,14-dioxo-3,4,12,14-tetrahydro-1-pyrano[3′,4′:6,7]indolizino[1,2-b]quinolin-11-yl)-9,12-dimethyl-3,8,11,14-tetraoxo-5-oxa-2,7,10,13-tetraazahexadecan-15-yl)-6-(2-(methylsulfonyl)pyrimidin-5-yl)hex-5-ynamide (A-78)

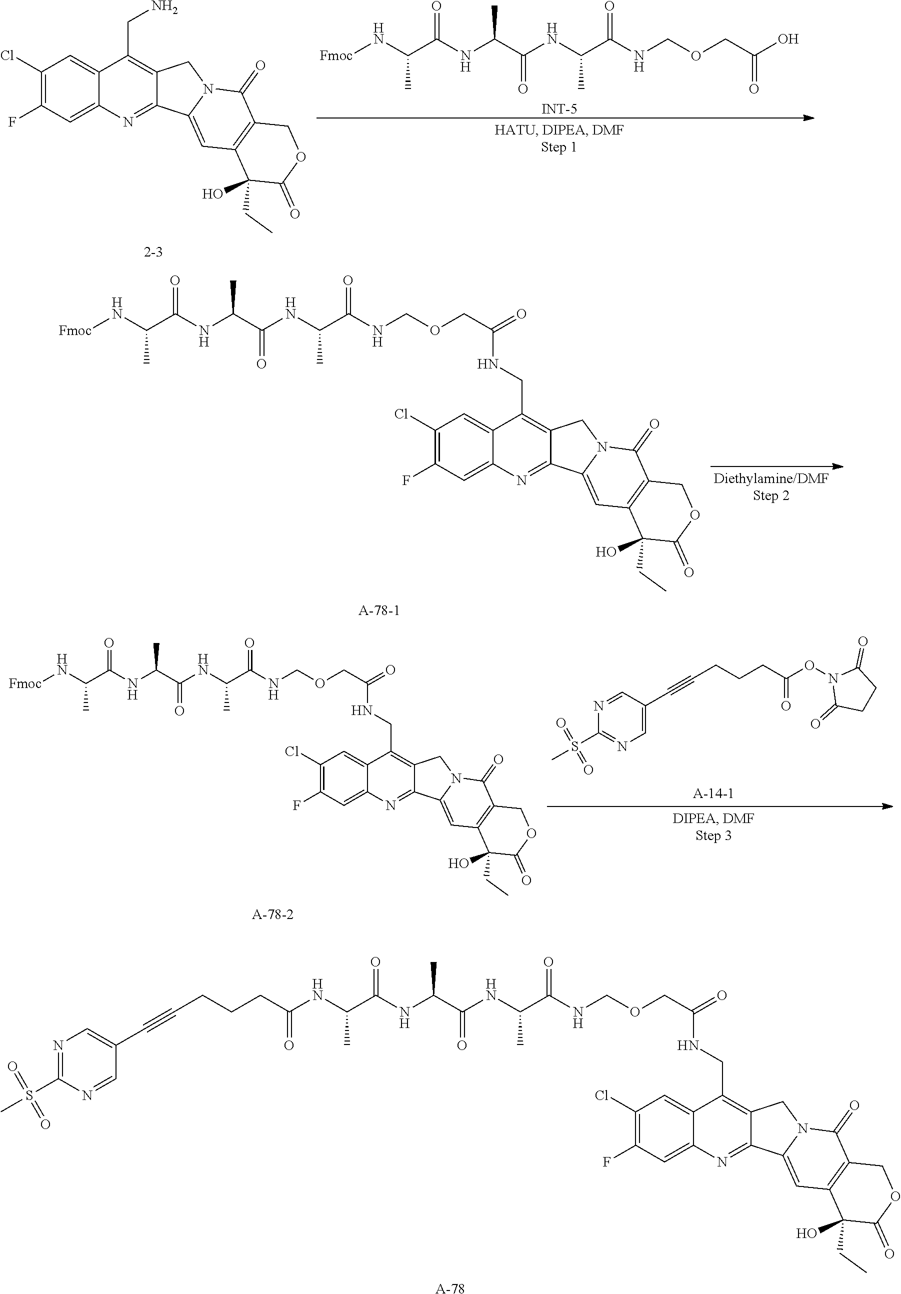

### Step 1: Preparation of (9H-fluoren-9-yl)methyl ((9S,12S,15S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-9,12-dimethyl-3,8,11,14-tetraoxo-5-oxa-2,7,10,13-tetraazahexadecan-15-yl)carbamate (A-78-1)

Compound 2-3 (50 mg, 116.33 μmol) and compound INT-5 (66.03 mg, 122.14 μmol) were dissolved in N,N-dimethylformamide (1 mL), then added with HATU (48.62 mg, 127.96 μmol) and N,N-diisopropylethylamine (37.58 mg, 290.81 μmol), stirred at room temperature for 1 hour. After the reaction was completed, the reaction solution was concentrated, and the resultant crude product was purified by high performance liquid chromatography and freeze-dried to obtain 100 mg of the title compound.

Its purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 28 |
| 2.00 | 20 | 80 | 28 |
| 18.00 | 80 | 20 | 28 |

### Step 2: Preparation of (S)-2-amino-N-((9S,12S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-9-methyl-3,8,11-trioxo-5-oxa-2,7,10-triazadecan-12-yl)propanamide (A-78-2)

Compound A-78-1 (100 mg, 105.00 μmol) was dissolved in N,N-dimethylformamide (1 mL), added with diethylamine (0.5 mL), and reacted at room temperature for 1 hour. After the reaction was completed, the reaction solution was concentrated under reduced pressure to obtain a crude product, which was directly used in the next step.

### Step 3: Preparation of N-((9S,12S,15S)-1-((S)-9-chloro-4-ethyl-8-fluoro-4-hydroxy-3,14-dioxo-3,4,12,14-tetrahydro-1-pyrano[3′,4′:6,7]indolizino[1,2-b]quinolin-11-yl)-9,12-dimethyl-3,8,11,14-tetraoxo-5-oxa-2,7,10,13-tetraazahexadecan-15-yl)-6-(2-(methylsulfonyl)pyrimidin-5-yl)hex-5-ynamide (A-78)

Compound A-14-1 (17.66 mg, 48.39 μmol) and N,N-diisopropylethylamine (12.48 mg, 0.226 mmol) were dissolved in N,N-dimethylformamide (1 mL) containing crude A-78-2, and the reaction system was stirred at room temperature for 1 hour. After the reaction was completed, the reaction solution was concentrated under reduced pressure to obtain a crude product, which was purified by high-performance liquid chromatography and freeze-dried to obtain 5 mg of the title compound.

Its structural characterization data are as follows:

MS m/z (ESI): 981.7 [M+H]⁺.

Its purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 28 |
| 2.00 | 20 | 80 | 28 |
| 18.00 | 80 | 20 | 28 |

### Example 20: N—((S)-12-benzyl-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-80)

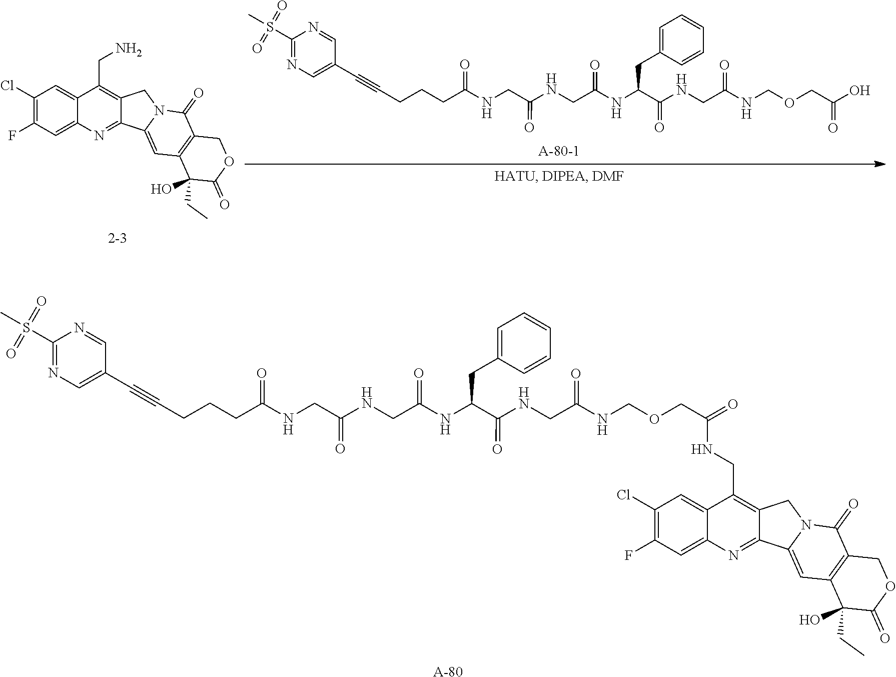 [SMILES: CC[C@@]1(O)C(=O)OCc2c1cc1n(c2=O)Cc2c-1nc1cc(F)c(Cl)cc1c2CN ; CS(=O)(=O)c1ncc(C#CCCCC(=O)NCC(=O)NCC(=O)N[C@@H](Cc2ccccc2)C(=O)NCC(=O)NCOCC(=O)O)cn1 ; CC[C@@]1(O)C(=O)OCc2c1cc1n(c2=O)Cc2c-1nc1cc(F)c(Cl)cc1c2CNC(=O)COCNC(=O)CNC(=O)[C@H](Cc1ccccc1)NC(=O)CNC(=O)CNC(=O)CCCC#Cc1cnc(S(C)(=O)=O)nc1 ; "A-80"]

Compound 2-3 (30 mg, 69.80 μmol) and compound A-80-1 (51.72 mg, 76.77 μmol, prepared according to the method described on page 92 of WO2022253035) was dissolved in N,N-dimethylformamide (1 mL), then added with HATU (29.17 mg, 76.77 μmol) and N,N-diisopropylethylamine (22.55 mg, 174.49 μmol), and stirred at room temperature for 1 hour. After the reaction was completed, the reaction solution was directly purified by preparative high performance liquid chromatography and freeze-dried to obtain 10 mg of the title compound.

Its structural characterization data are as follows:

MS m/z (ESI): 1087.3 [M+H]⁺

Its purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 28 |
| 2.00 | 20 | 80 | 28 |
| 18.00 | 80 | 20 | 28 |

### Example 21:(2R,3R,4R,5S,6R)-6-(4-(((((S)-9-Chloro-4-ethyl-8-fluoro-4-hydroxy-3,4,12,14-tetrahydro-1H-pyrano[3′,4′:6,7]indolizino[1,2-b]quinolin-11-yl)methyl)carbamoyl)oxy)-methyl)-2-(2-(2-(N-(6-(2-(methylsulfonyl)pyrimidin-5-yl)hex-5-ynamido)ethylamino)ethoxy)-acetamido)phenoxy)-3,4,5-trihydroxy-tetrahydro-2H-pyran-2-carboxylic acid (A-84)

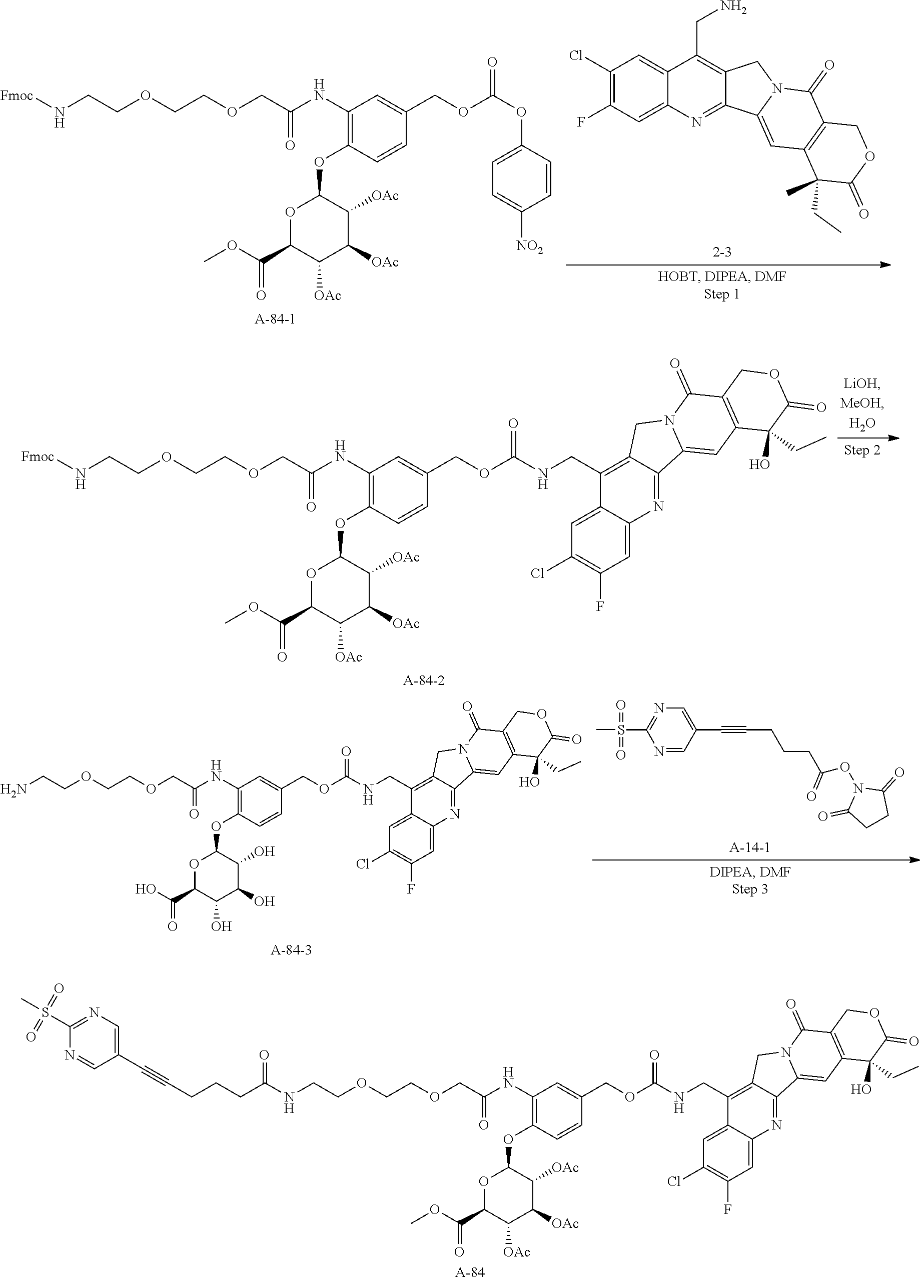

### Step 1: Preparation of (2R,3S,4R,5R,6R)-2-(2-(8-(9-fluorenylmethoxycarbonylamino)-3,6-dioxa-octanamido)-4-((((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamoyl)oxy)-methyl)phenoxy)-6-(methoxycarbonyl)-tetrahydro-2H-pyran-3,4,5-triacetate (A-84-2)

Compound 2-3 (30 mg, 69.80 μmol) and compound A-84-1 (75.85 mg, 76.77 μmol, prepared according to the method described on page 81 of WO2022253035) was dissolved in N,N-dimethylformamide (1 mL), then added with HOBT (11.32 mg, 83.75 μmol) and N,N-diisopropylethylamine (22.55 mg, 174.49 μmol), and stirred at room temperature for 1 hour. After the reaction was completed, the reaction solution was spin-dried to obtain the crude product A-84-2, which was directly subjected to the next step of reaction.

### Step 2: Preparation of (2R,3R,4R,5S,6R)-6-(2-(2-(2-(2-aminoethyl)ethoxy)acetamido)-4-((((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11yl)methyl)carbamoyl)oxy)methyl)phenoxy)-3,4,5-trihydroxytetrahydro-2H-pyran-2-carboxylic acid (A-84-3)

A-84-2 was dissolved in MeOH (1 mL) and water (0.5 mL), added with LiOH·H₂O (26.28 mg, 625.67 μmol), reacted at room temperature for 4 hours. After the reaction was completed, the reaction solution was directly purified by preparative high performance liquid chromatography and freeze-dried to obtain the title compound A-84-3 (20 mg).

Its purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 28 |
| 2.00 | 20 | 80 | 28 |
| 18.00 | 80 | 20 | 28 |

### Step 3: (2R,3R,4R,5S,6R)-6-(4-((((((S)-9-chloro-4-ethyl-8-fluoro-4-hydroxy-3,4,12,14-tetrahydro-1H-pyrano[3′,4′:6,7]indolizino[1,2-b]quinolin-11-yl)methyl)carbamoyl)oxy)methyl)-2-(2-(2-(N-(6-(2-(methylsulfonyl)pyrimidin-5-yl)hex-5-ynamido)ethylamino)ethoxy)-acetamido)phenoxy)-3,4,5-trihydroxy-tetrahydro-2H-pyran-2-carboxylic acid (A-84)

A-84-3 (20 mg, 21.83 μmol), A-14-1 (7.98 mg, 21.83 μmol) and N,N-diisopropylethylamine (8.46 mg, 65.49 μmol) were dissolved in DMF (1 mL), and the reaction solution was stirred at room temperature for 1 hour. After the reaction was completed, the reaction solution was directly purified by preparative high performance liquid chromatography and freeze-dried to obtain 3.42 mg of the title compound.

Its structural characterization data are as follows:

MS m/z (ESI): 1168.2 [M+H]⁺

Its purification method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 28 |
| 2.00 | 30 | 70 | 28 |
| 18.00 | 90 | 10 | 28 |

### Example 22: N—((S)-1-(((S)-1-((((S)-1-(((((S)-4-ethyl-11-(2-(N-isopropylmethylsulfonamido)-ethyl)-3,14-dioxo-3,4,12,14-tetrahydro-1H-pyrano[3′,4′:6,7]indolizino-[1,2-b]quinolin-4-yl)oxy)methyl)amino)-1-oxopropyl-2-yl)amino)-1-oxopropyl-2-yl)amino-1-oxopropyl-2-yl)-6-(2-(methylsulfonyl)pyrimidin-5-yl)hex-5-ynamide (A-88)

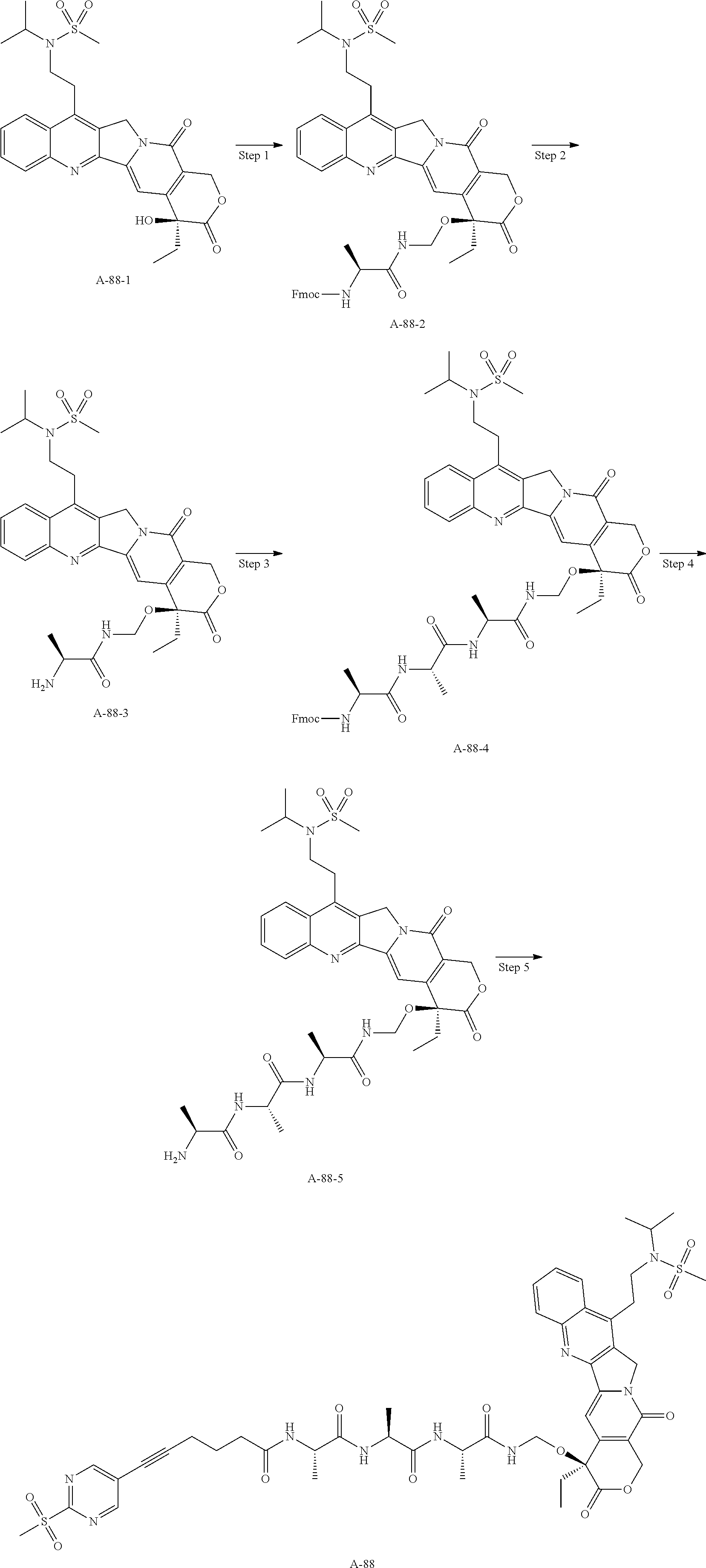

### Step 1: Preparation of (9H-fluoren-9-yl)methyl ((S)-1-(((((S)-4-ethyl-11-(2-(N-isopropylmethylsulfonamido)ethyl)-3,14-dioxo-3,4,12,14-tetrahydro-1H-pyrano[3′,4′:6,7]-indolizino[1,2-b]quinolin-4-yl)oxy)methyl)amino)-1-oxopropyl-2-yl)carbamate (A-88-2)

(S)—N-(2-(4-Ethyl-4-hydroxy-3,14-dioxo-3,4,12,14-tetrahydro-1H-pyrano[3′,4′:6,7]-indolizino[1,2-b]quinolin-11-yl)ethyl)-N-isopropylmethanesulfonamide (300 mg, 586.41 μmol), methyl (S)-(2-((((9H-fluoren-9-yl)methoxy)carbonyl)amino)propionamido)acetate (224.25 mg, 586.41 μmol) and anhydrous zinc acetate (107.59 mg, 586.41 μmol) were added to NMP (7 mL), subjected to nitrogen replacement three times, stirred at room temperature for 10 minutes to completely dissolve, and then placed in an oil bath preheated to 115° C., and reacted for 2 hours. The feeding of one batch materials was repeated. After the reaction was completed, the two batches of reaction liquid were combined. The reaction liquid was added with 50 mL of water, and a large amount of solid precipitated. After stirring for ten minutes, filtration was carried out, and the filter cake was collected. The filter cake was ultrasonically dispersed into 20 mL of methanol and filtered, and the resultant filter cake was washed with an appropriate amount of methanol (the filter cake was the unreacted raw materials). The filtrate was concentrated under reduced pressure to obtain a crude product, which was purified by preparative high performance liquid chromatography and freeze-dried to obtain the title compound (30 mg, 35.97 μmol).

Its structural characterization data are as follows:

MS m/z (ESI): 834.4 [M+H]⁺

The preparation method is as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 50 | 50 | 30 |
| 2.00 | 50 | 50 | 30 |
| 18.00 | 95 | 5 | 30 |

### Step 2: Preparation of (S)-2-amino-N—((((S)-4-ethyl-11-(2-(N-isopropylmethylsulfonamido) ethyl)-3,14-dioxo-3,4,12,14-tetrahydro-1H-pyrano[3′,4′:6,7]indolizino[1,2-b]quinolin-4-yl)oxy)methyl)propanamide (A-88-3)

(9H-Fluoren-9-yl)methyl ((S)-1-(((((S)-4-ethyl-11-(2-(N-isopropylmethylsulfonamido) ethyl)-3,14-dioxo-3,4,12,14-tetrahydro-1H-pyrano[3′,4′:6,7]indolizino[1,2-b]quinolin-4-yl)oxy)methyl)amino)-1-oxopropyl-2-yl)carbamate (25 mg, 29.98 μmol) was dissolved in DMF (5 mL), added with diethylamine (0.5 mL), and reacted under stirring at room temperature for 1 hour. After the reaction was completed, the reaction solution was directly freeze-dried to remove the residual diethylamine under reduced pressure, and the resultant crude product in the form of DMF solution was directly used in the next step.

Its structural characterization data are as follows:

MS m/z (ESI): 612.2 [M+H]⁺

### Step 3: Preparation of (9H-fluoren-9-yl)methyl ((S)-1-(((S)-1-(((S)-1-((((S)-4-ethyl-11-(2-(N-isopropylmethylsulfonamido)ethyl)-3,14-dioxo-3,4,12,14-tetrahydro-1H-pyrano[3′,4′:6,7]-indolizino[1,2-b]quinolin-4-yl)oxy)methyl)amino)-1-oxopropyl-2-yl)amino)-1-oxopropyl-2-yl)amino)-1-oxopropyl-2-yl)aminocarbonate (A-88-4)

To the reaction mixture containing (S)-2-amino-N—((((S)-4-ethyl-11-(2-(N-isopropylmethylsulfonamido)ethyl)-3,14-dioxo-3,4,12,14-tetrahydro-1H-pyrano[3′,4′:6,7]-indolizino[1,2-b]quinolin-4-yl)oxy)methyl)propanamide (20 mg, 32.70 μmol) in the previous step, (((9H-fluoren-9-yl)methoxy)carbonyl)-L-alanine-L-alanine (12.50 mg, 32.70 μmol) and DMF (1 mL) were added, then added with HATU (14.92 mg, 39.23 μmol) and DIPEA (8.45 mg, 65.39 μmol), and stirred at room temperature for 1 hour. After the reaction was completed, the reaction solution was directly used in the next step without treatment.

Its structural characterization data are as follows:

MS m/z (ESI): 976.5 [M+H]⁺

### Step 4: Preparation of (S)-2-amino-N—((S)-1-(((S)-1-(((((S)-4-ethyl-11-(2-(N-isopropylmethylsulfonylamino)ethyl))-3,14-dioxo-3,4,12,14-tetrahydro-1H-pyrano[3′,4′:6,7]-indolizino[1,2-b]quinolin-4-yl)oxy)methyl)amino)-1-oxopropyl-2-yl)amino)-1-oxopropyl-2-yl)propanamide (A-88-5)

Diethylamine (0.5 mL) was added to the reaction solution containing (9H-fluoren-9-yl)methyl ((S)-1-(((S)-1-(((S)-1-((((S)-4-ethyl-11-(2-(N-isopropylmethylsulfonamido) ethyl)-3,14-dioxo-3,4,12,14-tetrahydro-1H-pyrano[3′,4′:6,7]indolizino[1,2-b]quinolin-4-yl)oxy)methyl)amino)-1-oxopropyl-2-yl)amino)-1-oxopropyl-2-yl)amino)-1-oxopropyl-2-yl)aminocarbonate (38 mg, 38.93 μmol) in the previous step, and reacted under stirring at room temperature for 1 hour. After the reaction was completed, the reaction solution was lyophilized to remove diethylamine and part of the solvent, and the resultant crude product was purified by reverse phase column chromatography (0.05% formic acid aqueous solution-acetonitrile=0 to 80%) and freeze-dried to obtain the title compound (15 mg, 18.75 μmol).

Its structural characterization data are as follows:

MS m/z (ESI): 754.4 [M+H]⁺

### Step 5: Preparation of N—((S)-1-(((S)-1-(((S)-1-((((S)-4-ethyl-11-(2-(N-isopropylmethylsulfonamido) ethyl)-3,14-dioxo-3,4,12,14-tetrahydro-1H-pyrano[3′,4′:6,7]-indolizino[1,2-b]quinolin-4-yl)oxy)methyl)amino)-1-oxopropyl-2-yl)amino)-1-oxopropyl-2-yl)amino-1-oxopropyl-2-yl)-6-(2-(methylsulfonyl)pyrimidin-5-yl)hex-5-ynamide (A-88)

(S)-2-Amino-N—((S)-1-(((S)-1-(((((S)-4-ethyl-11-(2-(N-isopropylmethylsulfonylamino)-ethyl))-3,14-dioxo-3,4,12,14-tetrahydro-1H-pyrano[3′,4′:6,7]indolizino[1,2-b]quinolin-4-yl)oxy)methyl)amino)-1-oxopropyl-2-yl)amino)-1-oxopropyl-2-yl)propanamide (9 mg, 11.25 μmol) was dissolved in DMF (1 mL), then added dropwise with DIPEA (4.36 mg, 33.75 μmol), and added with 2,5-dioxopyrrolidin-1-yl 6-(2-(methylsulfonyl)pyrimidin-5-yl)hex-5-ynoate (4.11 mg, 11.25 μmol). After the addition was completed, the reaction solution was stirred at room temperature for 1 hour. After the reaction was completed, the reaction solution was directly purified by preparative high performance liquid chromatography and freeze-dried to obtain the title compound (4.11 mg, 11.25 μmol).

Its structural characterization data are as follows:

MS m/z (ESI): 1004.4 [M+H]⁺

Its preparation method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 3 | 97 | 30 |
| 2.00 | 3 | 97 | 30 |
| 18.00 | 70 | 30 | 30 |

### Example 23: N—((S)-7-Benzyl-1-(((S)-4-ethyl-11-(2-(N-isopropylmethylsulfonamido)-ethyl)-3,14-dioxo-3,4,12,14-tetrahydro-1H-pyrano[3′,4′:6,7]indolizino[1,2-b]quinolin-4-yl)oxy)-3,6,9,12-tetraoxo-2,5,8,11-tetraazatridecan-13-yl)-6-(2-(methylsulfonyl)pyrimidin-5-yl)hex-5-ynamide (A-89)

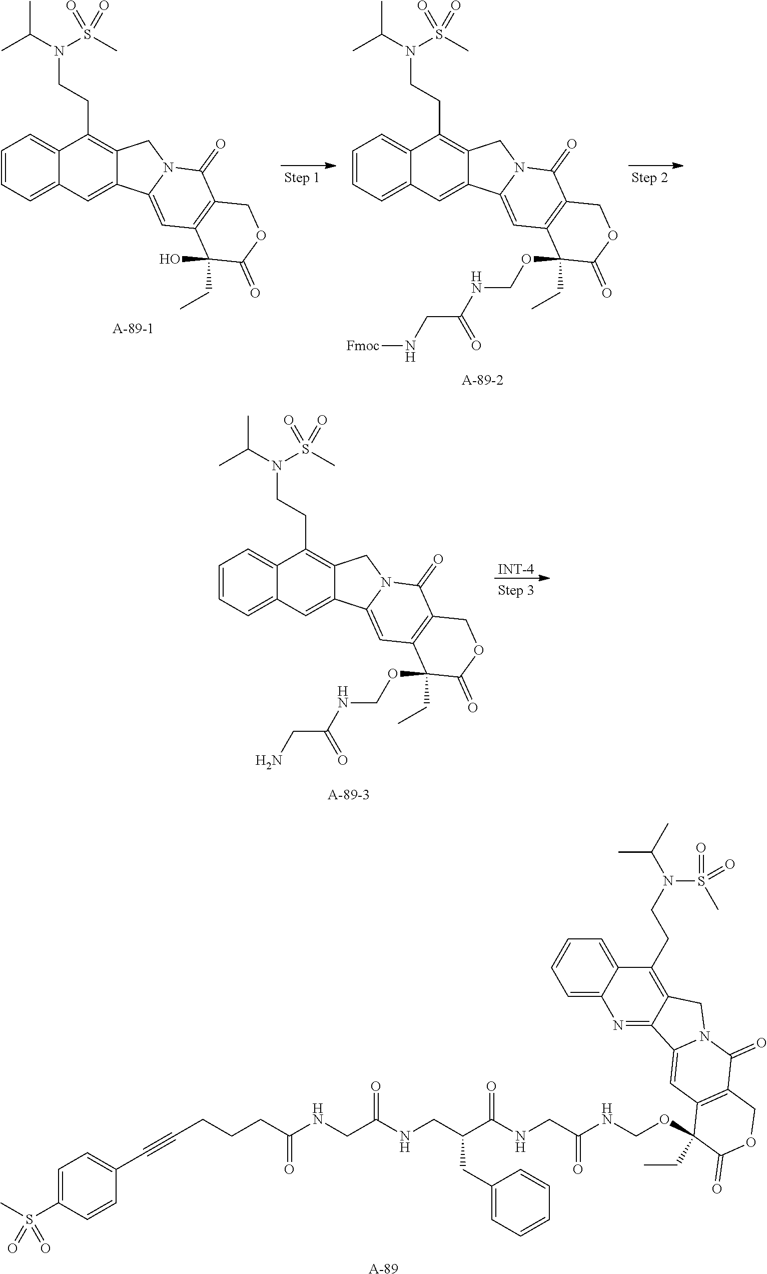

### Step 1: Preparation of (9H-fluoren-9-yl)methyl (S)-(2-((((4-ethyl-11-(2-(N-isopropylmethylsulfonamido)ethyl)-3,14-dioxo-3,4,12,14-tetrahydro-1H-pyrano[3′,4′:6,7]-indolizino[1,2-b]quinolin-4-yl)oxy)methyl)amino)-2-oxoethyl)carbamate (A-89-2)

(S)—N-(2-(4-ethyl-4-hydroxy-3,14-dioxo-3,4,12,14-tetrahydro-1H-pyrano[3′,4′:6,7]-indolizino[1,2-b]quinolin-11-yl)ethyl)-N-isopropylmethanesulfonamide (300 mg, 586.41 μmol), (2-((((9H-fluoren-9-yl)methoxy)carbonyl)amino)acetamido)methyl acetate (432.05 mg, 1.17 mmol) and anhydrous zinc acetate (107.59 mg, 586.41 μmol) were added to NMP (7 mL), subjected to nitrogen replacement three times, stirred at room temperature until the solid was completely dissolved, then placed in an oil bath preheated to 115° C. and reacted for 2 hours. After the reaction was completed, the reaction solution was added dropwise with 50 mL of water under stirring. A large amount of solid precipitated. After stirring for ten minutes, the mixture was filtered and the filter cake was collected. 10 mL of methanol was added to the filter cake to disperse the solid, and the mixture was filtered after stirring for 10 minutes. The resultant filtrate was collected and concentrated under reduced pressure to obtain the crude product of the title compound (100 mg, 48.79 μmol), which was directly used in the next reaction without further purification.

Its structural characterization data are as follows:

MS m/z (ESI): 820.4 [M+H]⁺

### Step 2: Preparation of (S)-2-amino-N—(((4-ethyl-11-(2-(N-isopropylmethylsulfonylamino) ethyl)-3,14-dioxo-3,4,12,14-tetrahydro-1H-pyranyl[3′,4′:6,7]indolizino[1,2-b]quinolin-4-yl)oxy)methyl)acetamide (A-89-3)

The crude product of (9H-fluoren-9-yl)methyl (S)-(2-((((4-ethyl-11-(2-(N-isopropylmethylsulfonylamino)ethyl)-3,14-dioxo-3,4,12,14-tetrahydro-1H-pyranyl[3′,4′:6,7]-indolizino[1,2-b]quinolin-4-yl)oxy)methyl)amino)-2-oxoethyl)carbamate (100 mg, 48.79 μmol) in the previous step was dissolved in DMF (3 mL), added with diethylamine (0.5 mL), and stirred at room temperature for 1 hour. After the reaction was completed, the reaction solution was directly purified by reverse phase column chromatography (0.05% formic acid aqueous solution-acetonitrile=0 to 90%) and freeze-dried to obtain the title compound (13 mg, 20.20 μmol).

Its structural characterization data are as follows:

MS m/z (ESI): 598.3 [M+H]⁺

### Step 3: Preparation of N—((S)-7-benzyl-1-(((S)-4-ethyl-11-(2-(N-isopropylmethyl-sulfonylamido)ethyl)-3,14-dioxo-3,4,12,14-tetrahydro-1H-pyrano[3′,4′:6,7]indolizino[1,2-b]quinolin-4-yl)oxy)-3,6,9,12-tetraoxo-2,5,8,11-tetraazatridecan-13-yl)-6-(2-(methylsulfonyl)-pyrimidin-5-yl)hex-5-ynamide (A-89)

(6-(2-(methylsulfonyl)pyrimidin-5-yl)hex-5-ynoyl)glycylglycyl-L-phenylalanine (8.23 mg, 15.54 μmol) and (S)-2-amino-N—(((4-ethyl-11-(2-(N-isopropylmethylsulfonylamino)ethyl)-3,14-dioxo-3,4,12,14-tetrahydro-1H-pyranyl[3′,4′:6,7]indolizino[1,2-b]quinolin-4-yl)oxy)-methyl)acetamide (10.00 mg, 15.54 μmol) were dissolved in DMF (1 mL), added with DIPEA (6.02 mg, 46.61 μmol), then added with HATU (7.09 mg, 18.64 μmol), and stirred at room temperature for 2 hours. After the reaction was completed, the reaction solution was directly purified by preparative high performance liquid chromatography and freeze-dried to obtain the title compound (1.78 mg, 1.57 μmol).

Its structural characterization data are as follows:

MS m/z (ESI): 1110.4 [M+H]⁺

Its preparation method was as follows:

Chromatographic column: Waters SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 2.00 | 10 | 90 | 30 |
| 24.00 | 90 | 10 | 30 |

### Example 24: (10S,19S)-10-Benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-19-(6-(2-(methylsulfonyl)pyrimidin-5-yl)hex-5-ynamido)-1,6,9,12,15,18,22-heptaoxo-3-oxa-5,8,11,14,17,23-hexaazapentacosane-25-sulfonic acid (B-3)

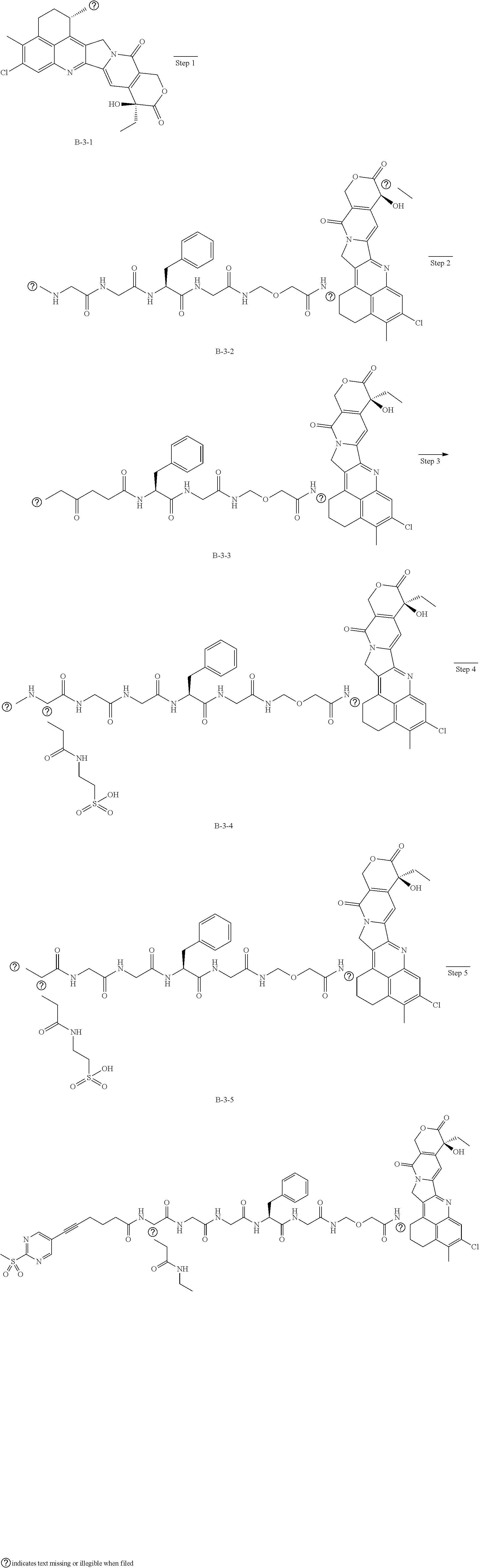

### Step 1: Preparation of (9H-fluoren-9-yl)methyl ((S)-10-benzyl-1-((1S,9S)-5-chloro-9-ethyl-9-hydroxy-4-methyl-10,13-dioxo-2,3,9,10,13,15-hexahydro-1H,12H-benzo[de]pyrano-[3′,4′:6,7]indolizino[1,2-b]quinolin-1-yl)amino)-1,6,9,12,15-pentaoxo-3-oxa-5,8,11,14-tetraazahexadecan-16-yl)carbamate (B-3-2)

(S)-11-Benzyl-1-(9H-fluoren-9-yl)-3,6,9,12,15-pentaoxo-2,18-dioxa-4,7,10,13,6-pentaazaeicosan-20-oic acid (320 mg, 455.38 μmol) and (1S,9S)-1-amino-5-chloro-9-ethyl-9-hydroxy-4-methyl-1,2,3,9,12,15-hexahydro-10H,13H-benzo[de]pyrano[3′,4′:6,7]indolizino-[1,2-b]quinolin-10,13-dione (227.38 mg, 500.92 μmol) were dissolved in DMF (6 mL), added with HATU (190.35 mg, 500.92 μmol) and DIPEA (176.56 mg, 1.37 mmol) in sequence, and reacted under stirring at room temperature for 2 hours. The reaction system was added with water (70 mL), and extracted with ethyl acetate three times (15 mL×3). The organic phases were combined and washed three times with saturated sodium chloride aqueous solution (10 mL×3), dried over anhydrous sodium sulfate, filtered and concentrated to obtain the title compound (360 mg, 316.17 μmol), which was used directly in the next step without purification.

Its structural characterization data are as follows:

MS m/z (ESI): 1079.4[M+H]⁺

### Step 2: Preparation of (S)-2-(2-(2-aminoacetamido)acetamido)-N-(2-(((2-(((1S,9S)-5-chloro-9-ethyl-9-hydroxy-4-methyl-10,13-dioxo-2,3,9,13,15-hexahydro-1H,12H-benzo[de]-pyrano[3′,4′:6,7]indolizino[1,2-b]quinolin-1-yl)amino)-2-oxoethoxy)methyl)amino)-2-oxoethyl)-3-phenylpropanamide (B-3-3)

(9H-Fluoren-9-yl)methyl ((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino-[1,2-b]quinolin-1-yl)amino)-1,6,9,12,15-pentaoxo-3-oxa-5,8,11,14-tetraazahexadecan-16-yl)carbamate (332.46 mg, 307.39 μmol, FR) was dissolved in DMF (5 mL), added with diethylamine (307.39 μmol, 1.5 mL), then reacted under stirring at room temperature for 30 minutes. The reaction solution was directly purified by reverse phase column chromatography (0.05% formic acid aqueous solution-acetonitrile=0 to 70%) and freeze-dried to obtain the title compound (115 mg, 133.83 μmol).

MS m/z (ESI): 857.3[M+H]⁺

### Step 3: Preparation of (10S,19S)-19-((((9H-fluoren-9-yl)methoxy)carbonyl)amino)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18,22-heptaoxo-3-oxa-5,8,11,14,17,23-hexaazapentacosane-25-sulfonic acid (B-3-4)

N²-(((9H-fluoren-9-yl)methoxy)carbonyl)-N⁵-(2-sulfoethyl)-L-glutamine (19 mg, 39.87 μmol) and (S)-2-(2-(2-aminoacetamido)acetamido)-N-(2-(((2-(((1S,9S)-5-chloro-9-ethyl-9-hydroxy-4-methyl-10,13-dioxo-2,3,9,13,15-hexahydro-1H,12H-benzo[de]pyrano[3′,4′:6,7]-indolizino[1,2-b]quinolin-1-yl)amino)-2-oxoethoxy)methyl)amino)-2-oxoethyl)-3-phenylpropanamide (30 mg, 33.21 μmol) were combined and dissolved in DMF (1.5 mL), added with DIPEA (34.34 mg, 265.68 μmol), stirred for 10 minutes, then added with HATU (16.42 mg, 43.17 μmol), and reacted under stirring at room temperature for 2 hours. After the reaction was completed, the reaction solution was directly used in the next reaction without any treatment.

Its structural characterization data are as follows:

MS m/z (ESI): 1315.7[M+H]⁺, 1333.7[M+H+H₂O]⁺

### Step 4: Preparation of (10S,19S)-19-amino-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-1,6,9,12,15,18,22-heptaoxo-3-oxa-5,8,11,14,17,23-hexaazapentacosane-25-sulfonic acid (B-3-5)

Diethylamine (0.5 mL) was directly added to the reaction solution containing (10S,19S)-19-((((9H-fluoren-9-yl)methoxy)carbonyl)amino)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-1,6,9,12,15,18,22-heptaoxo-3-oxa-5,8,11,14,17,23-hexaazapentacosane-25-sulfonic acid (43 mg, 32.68 μmol) and DMF (1.5 mL) in the previous step. After the addition was completed, the reaction was carried out under stirring at room temperature for 0.5 hours. After the reaction was completed, the reaction solution was directly purified by reverse phase column chromatography (0.05% formic acid aqueous solution-acetonitrile=0 to 50%) and freeze-dried to obtain the title compound (20 mg, 17.55 μmol).

Its structural characterization data are as follows:

MS m/z (ESI): 1093.5[M+H]⁺

### Step 5: Preparation of (10S,19S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-19-(6-(2-(methylsulfonyl)pyrimidin-5-yl)hex-5-ynamido)-1,6,9,12,15,18,22-heptaoxo-3-oxa-5,8,11,14,17,23-hexaazapentacosane-25-sulfonic acid (B-3)

(10S,19S)-19-Amino-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18,22-heptaoxo-3-oxa-5,8,11,14,17,23-hexaazapentacosane-25-sulfonic acid (15 mg, 13.16 μmol) was dissolved in DMF (1 mL), added with DIPEA (5.10 mg, 39.49 μmol), then added with 2,5-dioxopyrrolidin-1-yl 6-(2-(methylsulfonyl)pyrimidin-5-yl)hex-5-ynoate (6.00 mg, 16.42 μmol), and reacted under stirring at room temperature for 2 hours. After the reaction was completed, the reaction solution was directly separated and purified twice and freeze-dried to obtain the title compound (5.79 mg, 4.09 μmol).

Its structural characterization data are as follows:

MS m/z (ESI): 1343.3[M+H]⁺

The first separation and purification method was as follows:

Chromatographic column: Waters Xbridge Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 30 |
| 4.00 | 30 | 70 | 30 |
| 24.00 | 90 | 10 | 30 |

The second separation and purification method was as follows:

Chromatographic column: Waters Xbridge Prep C18
OBD (5 μm*19 mm*150 mm)
Mobile phase A: acetonitrile; Mobile phase B: water (0.05%
ammonium formate)

| | | | Flow rate |
|---|---|---|---|
| Time [min] | Mobile phase A [%] | Mobile phase B [%] | [mL/min] |
| 0.00 | 5 | 95 | 30 |
| 2.00 | 5 | 95 | 30 |
| 24.00 | 90 | 10 | 30 |

### Example 25: N—((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropane-2-yl)amino)-3-methyl-1-oxobutan-2-yl)-1-(6-(2-(methylsulfonyl)pyrimidin-5-yl)hex-5-ynamide)-3,6,9,12,15,18,21,24-octaoxaheptacosane-27-amide (A-156)

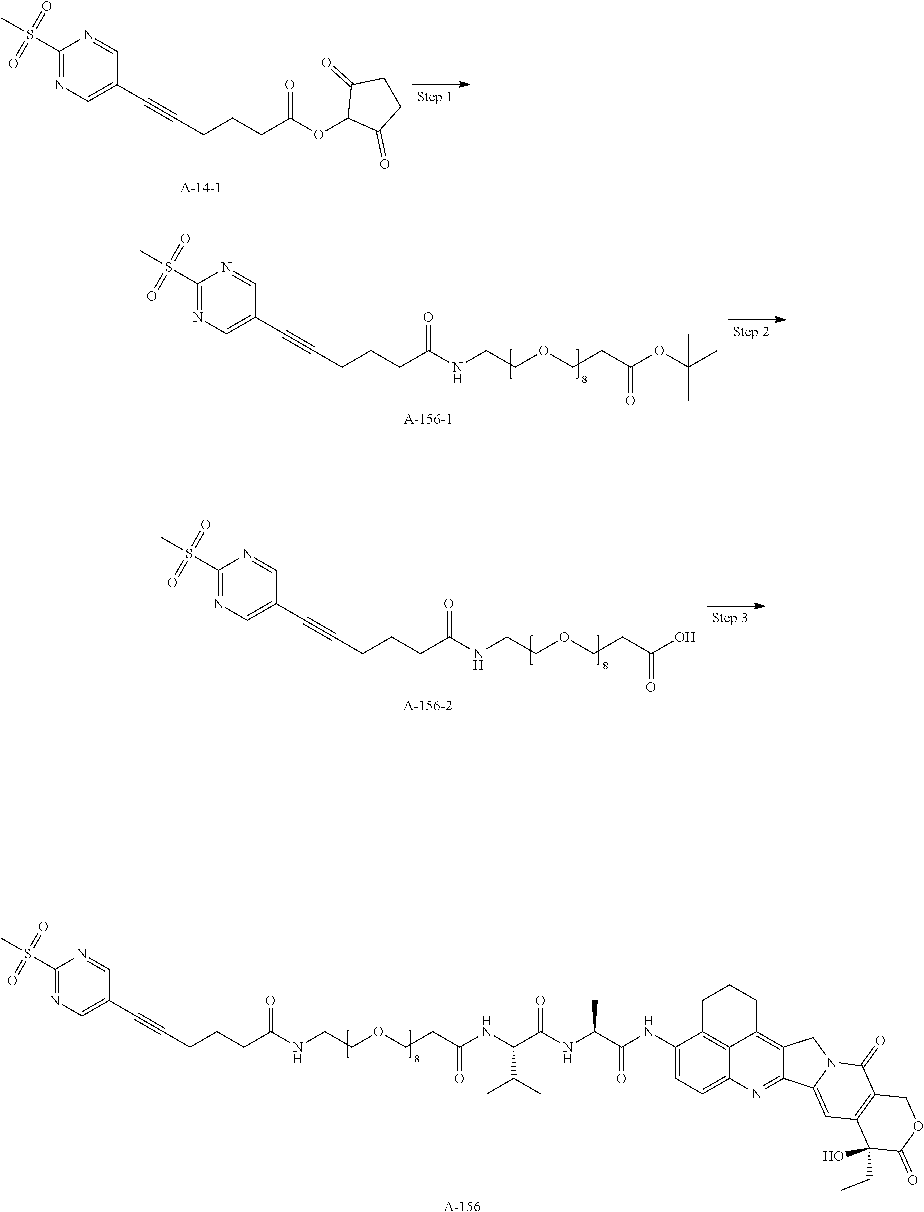

### Step 1: Preparation of tert-butyl 34-(2-(methylsulfonyl)pyrimidin-5-yl)-29-oxo-4,7,10,13,16,19,22,25-octaoxa-28-azatetratriacont-33-ynoate (A-156-1)

1-Amino-3,6,9,12,15,18,21,24-octaoxaheptacosan-27-oic acid (30.0 mg, 0.068 mmol) was dissolved in DCM (2 mL), added with DIPEA (26.3 mg, 0.2 mmol), and then added with A-14-1 (49.6 mg, 0.14 mmol). After the reaction was completed, the product was purified by flash column chromatography (C18, water/acetonitrile=0.5) and freeze-dried to obtain the title compound (37.2 mg, 0.05 mmol).

Its structural characterization data are as follows:

MS m/z (ESI): 748.2 [M+H]⁺

### Step 2: Preparation of 34-(2-(methylsulfonyl)pyrimidin-5-yl)-29-oxo-4,7,10,13,16,19,22,25-octaoxa-28-azatetratriacont-33-ynoic acid (A-156-2)

A-156-1 (37.2 mg, 0.05 mmol) was dissolved in DCM (3 mL), and added with 1.5 mL of trifluoroacetic acid. After the reaction was completed, the product was purified by flash column chromatography (C18, water/acetonitrile=0.4) and freeze-dried to obtain the title compound (28.4 mg, 0.04 mmol).

Its structural characterization data are as follows:

MS m/z (ESI): 692.1 [M+H]⁺

### Step 3: Preparation of N—((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropane-2-yl)amino)-3-methyl-1-oxobutan-2-yl)-1-(6-(2-(methylsulfonyl)pyrimidin-5-yl)hex-5-ynamide)-3,6,9,12,15,18,21,24-octaoxaheptacosane-27-amide (A-156)

A-156-2 (28.4 mg, 0.04 mmol) and (S)-2-amino-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropane-2-yl)-3-methylbutanamide (22.5 mg, 0.04 mmol) were dissolved in DMF (2 mL), added with 2-(7-azobenzotriazole)-N,N,N′,N′-tetramethyluronium hexafluorophosphate (19.9 mg, 0.05 mmol) and DIPEA (13.5 mg, 0.1 mmol), and stirred at room temperature for 1 hour. After the reaction was completed, the reaction solution was directly purified by preparative high performance liquid chromatography and freeze-dried to obtain the title compound (18.2 mg, 14.6 μmol).

Its structural characterization data are as follows:

MS m/z (ESI): 1247.3 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 2.00 | 10 | 90 | 30 |
| 20.00 | 70 | 30 | 30 |

### Example 26: N-(2-((2-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropane-2-yl)amino)-3-methyl-1-oxobutan-2-yl)amino)-2-oxoethyl)amino)-2-oxoethyl)-6-(2-(methylsulfonyl)pyrimidin-5-yl)hex-5-ynamide (A-167)

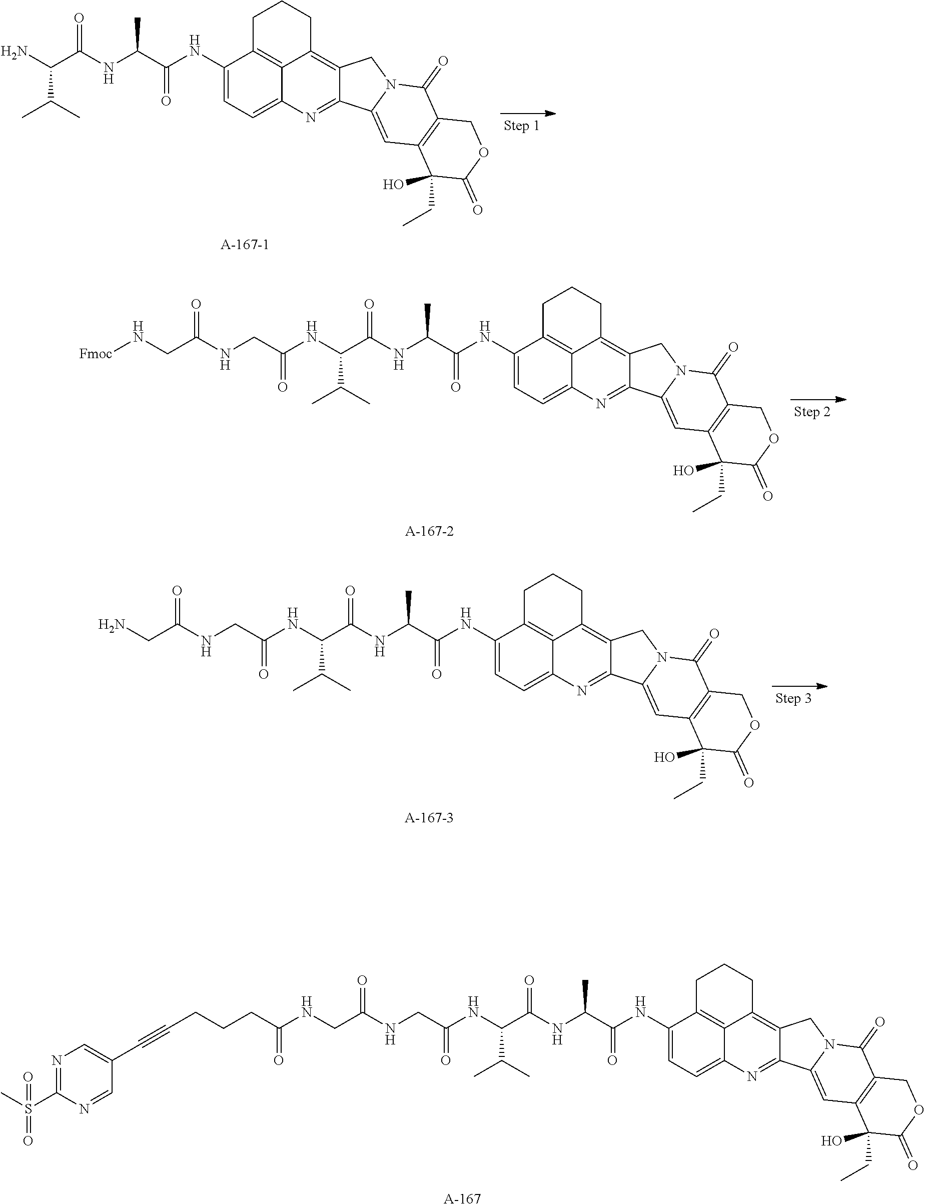

### Step 1: Preparation of (9H-fluoren-9-yl)methyl (2-((2-(((S)-1-(((S)-1-(((S)-9-ethyl-9-hydroxy-10,13-dioxo-2,3,9,10,13,15-hexahydro-1H,12H-benzo[de]pyrano[3′,4′:6,7]-indolizino [1,2-b]quinolin-4-yl)amino)-1-oxopropan-2-yl)amino)-3-methyl-1l-oxobutan-2-yl)amino)-2-oxoethyl)amino)-2-oxoethyl)carbamate (A-167-2)

(((9H-Fluoren-9-yl)methoxy)carbonyl)glycylglycine (10.9 mg, 0.03 mmol), (S)-2-amino-N—((S)-1-(((S)-9-ethyl-9-hydroxy-10,13-dioxo-2,3,9,10,13,15-hexahydro-1H,12H-benzo[de]pyrano[3′,4′: 6,7]indolizino[1,2-b]quinolin-4-yl)amino)-1-oxopropan-2-yl)-3-methylbutanamide (21.0 mg, 0.03 mmol) were dissolved in DMF (2 mL), added with 2-(7-azobenzotriazole)-N,N,N′,N′-tetramethyluronium hexafluorophosphate (19.9 mg, 0.05 mmol) and DIPEA (13.5 mg, 0.1 mmol), and stirred at room temperature for 1 hour. After the reaction was completed, 10 mL of water was added, and solid precipitated. After filtration, the resultant filter cake was freeze-dried to obtain the title compound (23.9 mg, 0.025 mmol), which was used directly in the next step without purification.

Its structural characterization data are as follows:

MS m/z (ESI): 910.3 [M+H]⁺

### Step 2: Preparation of (S)-2-(2-(2-aminoacetamido)acetamido)-N—((S)-1-(((S)-9-ethyl-9-hydroxy-10,13-dioxo-2,3,9,10,13,15-hexahydro-1H,12H-benzo[de]pyrano[3′,4′:6,7]-indolizino[1,2-b]quinolin-4-yl)amino)-1-oxopropan-2-yl)-3-methylbutanamide (A-167-3)

Diethylamine (18.3 mg, 0.25 mmol) was added to A-167-2 (23.9 mg, 0.025 mmol) in DMF (2 mL), and stirred at room temperature for 1 hour. After the reaction was completed, the solvent was removed by a freeze dryer, 15 mL of a mixed solvent (EA/PE=1/3) was added for recrystallization for 2 hours, and the title compound (16.1 mg, 0.023 mmol) was obtained by filtration.

Its structural characterization data are as follows:

MS m/z (ESI): 688.2 [M+H]⁺

### Step 3: Preparation of N-(2-((2-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropane-2-yl)amino)-3-methyl-1-oxobutan-2-yl)amino)-2-oxoethyl)amino)-2-oxoethyl)-6-(2-(methylsulfonyl)pyrimidin-5-yl)hex-5-ynamide (A-167)

A-167-3 (16.1 mg, 0.023 mmol) was dissolved in DMF (2 mL), added with DIPEA (15.1 mg, 0.12 mmol), and added with A-14-1 (16.8 mg, 0.046 mmol). After the reaction was completed, the reaction solution was directly purified by preparative high performance liquid chromatography and freeze-dried to obtain the title compound (15.5 mg, 16.5 μmol).

Its structural characterization data are as follows:

MS m/z (ESI): 938.2 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 32 |
| 4.00 | 20 | 80 | 32 |
| 24.00 | 80 | 20 | 32 |

### Example 27: Preparation of 2,2′,2″-(10-((10S,19S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano-[3′,4′:6,7]-indolizino[1,2-b]quinolin-1-yl)amino)-19-(6-(2-(methylsulfonyl)pyrimidin-5-yl)hex-5-ynamide)-1,6,9,12,15,18,25-heptaoxo-3-oxa-5,8,11,14,17,24-hexaazahexacosan-26-yl)-1,4,7,10-tetraazacyclododecan-1,4,7-triyl)triacetic acid (B-8)

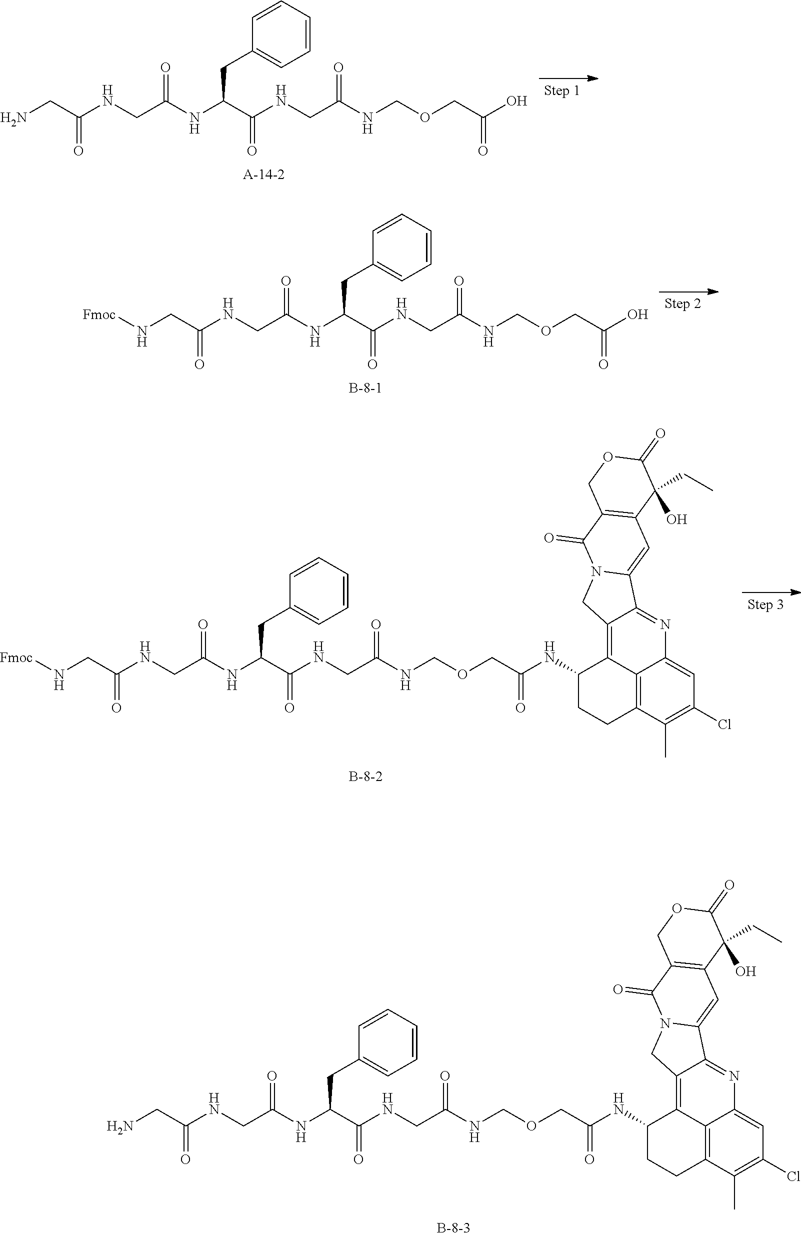

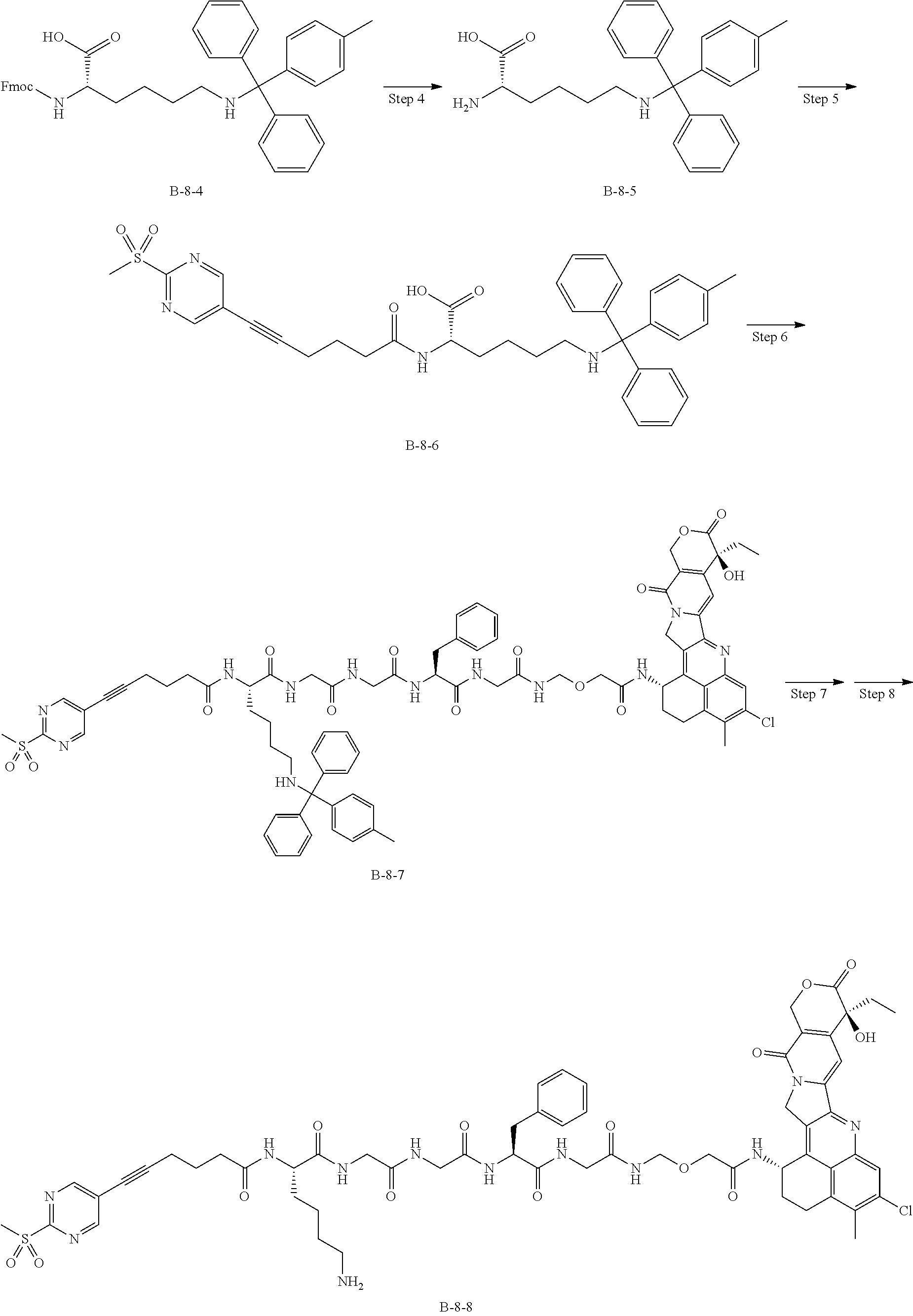

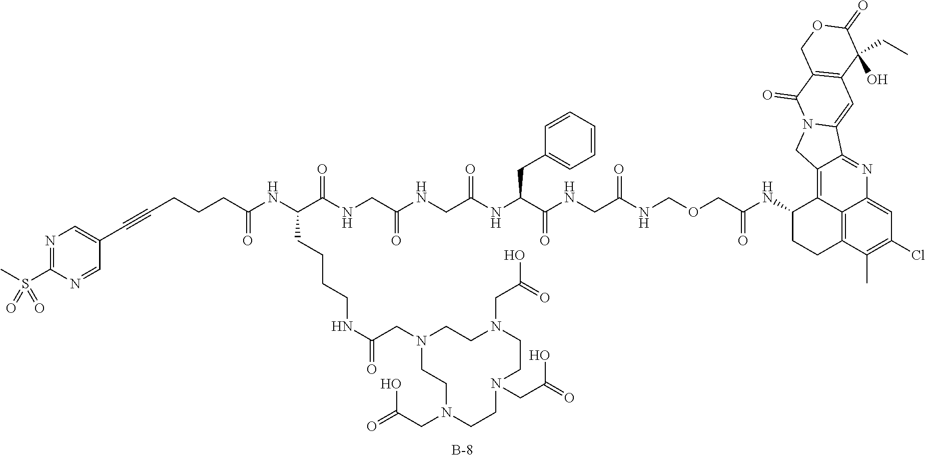

### Step 1: Preparation of (S)-11-benzyl-1-(9H-fluoren-9-yl)-3,6,9,12,15-pentaoxo-2,18-dioxa-4,7,10,13,16-pentaazaeicosan-20-oic acid (B-8-1)

(S)-16-Amino-10-benzyl-6,9,12,15-tetraoxo-3-oxa-5,8,11,14-tetraazahexadecanoic acid (1.2 g, 2.84 mmol) was dissolved in a mixed solvent of acetonitrile (8 mL) and water (8 mL), added with sodium bicarbonate (262.0 mg, 3.1 mmol) and 9-fluorenylmethyl-N-succinimidyl carbonate (1.15 g, 3.4 mmol). After the reaction was completed, the title compound (1.5 g, 2.33 mmol) was obtained by purification by flash column chromatography (C18, water/acetonitrile=0.6).

Its structural characterization data are as follows:

MS m/z (ESI): 663.1 [M+H₂O]⁺

### Step 2: Preparation of (9H-fluoren-9-yl)methyl ((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano-[3′,4′:6,7]indolizino[1,2-b]quinolin-1-yl)amino)-1,6,9,12,15-pentaoxo-3-oxa-5,8,11,14-tetraazahexadecane-16-yl)carbamate (B-8-2)

(S)-11-Benzyl-1-(9H-fluoren-9-yl)-3,6,9,12,15-pentaoxo-2,18-dioxa-4,7,10,13,16-pentaazaeicosan-20-oic acid (1.5 g, 2.33 mmol) and (1S,9S)-1-amino-9-ethyl-5-chloro-9-hydroxy-4-methyl-1,2,3,9,12,15-hexahydro-10H,13H-benzo[de]pyrano[3′,4′:6,7]indolizino-[1,2-b]quinolin-10,13-dione (1.16 g, 2.56 mmol) were dissolved in DMF (20 mL), added with 2-(7-azobenzotriazole)-N,N,N′,N′-tetramethyluronium hexafluorophosphate (972.40 mg, 2.56 mmol) and DIPEA (1.16 g, 8.96 mmol), and stirred at room temperature for 4 hours. After the reaction was completed, 100 mL of water was added, and solid precipitated. After filtration, the resultant filter cake was freeze-dried to remove water to obtain the title compound (1.3 g, 1.2 mmol).

Its structural characterization data are as follows:

MS m/z (ESI): 1079.3 [M+H]⁺

### Step 3: Preparation of (S)-2-(2-(2-aminoacetamido)acetamido)-N-(2-(((2-(((1S,9S)-5-chloro-9-ethyl-9-hydroxy-4-methyl)-10,13-dioxo-2,3,9,10,13,15-hexahydro-1H,12H-benzo-[de]pyrano[3′,4′:6,7]indolizino[1,2-b]quinolin-1-yl)amino)-2-oxoethoxy)methyl)amino)-2-oxoethyl)-3-phenylpropanamide (B-8-3)

(9H-Fluoren-9-yl)methyl ((S)-10-benzyl-1-(((1S,9S)-5-chloro-9-ethyl-9-hydroxy-4-methyl-10,13-dioxo)-2,3,9,10,13,15-hexahydro-1H,12H-benzo[de]pyrano[3′,4′:6,7]indolizino-[1,2-b]quinolin-1-yl)amino)-1,6,9,12,15-pentaoxo-3-oxa-5,8,11,14-tetraazahexadecan-16-yl)-carbamate (1.3 g, 1.2 mmol) was dissolved in DMF (10 mL), added with diethylamine (877.8 mg, 12.0 mmol), and stirred at room temperature for 1 hour. After the reaction was completed, the solvent was dried on a freeze dryer, 50 mL of a mixed solvent (EA/PE=1/1) was added for recrystallization for 16 hours, and the title compound (950.1 mg, 1.11 mmol) was obtained by filtration.

Its structural characterization data are as follows:

MS m/z (ESI): 857.2 [M+H]⁺

### Step 4: Preparation of N⁶-(diphenyl(p-tolyl)methyl)-L-lysine (B-8-5)

N²-(((9H-Fluoren-9-yl)methoxy)carbonyl)-N⁶-(diphenyl(p-tolyl)methyl)-L-lysine (150.0 mg, 0.24 mmol) was dissolved in DMF (2 mL), added with diethylamine (182.3 mg, 2.5 mmol), and stirred at room temperature for 1 hour. After the reaction was completed, 10 mL of mixed solvent (EA/PE=1/3) was added for recrystallization for 1 hour, and the title compound (90.1 mg, 0.22 mmol) was obtained by filtration.

### Step 5: Preparation of N⁶-(diphenyl(p-tolyl)methyl)-N²-(6-(2-(methylsulfonyl) pyrimidin-5-yl)hex-5-ynoyl)-L-lysine (B-8-6)

N⁶-(Diphenyl(p-tolyl)methyl)-L-lysine (90.1 mg, 0.22 mmol) was dissolved in DMF (3 mL), added with DIPEA (86.3 mg, 0.66 mmol), and added with 2,5-dioxopyrrolidin-1-yl 6-(2-(methylsulfonyl)pyrimidin-5-yl)hex-5-ynoate (160.6 mg, 0.44 mmol). After the reaction was completed, the title compound (75.2 mg, 0.11 mmol) was obtained after purification by flash column chromatography (C18, water/acetonitrile=0.5) and freeze-drying.

Its structural characterization data are as follows:

MS m/z (ESI): 653.2 [M+H]⁺

### Step 6: Preparation of N-((7S,16S)-16-benzyl-25-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino-[1,2-b]quinolin-1-yl)amino)-8,11,14,17,20,25-hexaoxo-1,1-diphenyl-1-(p-tolyl)-23-oxa-2,9,12,15,18,21-hexaazapentacosan-7-yl)-6-(2-(methylsulfonyl)pyrimidin-5-yl)hex-5-ynamide (B-8-7)

N⁶-(Diphenyl(p-tolyl)methyl)-N²-(6-(2-(methylsulfonyl)pyrimidin-5-yl)hex-5-ynoyl)-L-lysine (75.2 mg, 0.11 mmol) and (S)-2-(2-(2-aminoacetamido)acetamido)-N-(2-(((2-(((1S,9S)-5-chloro-9-ethyl-9-hydroxy-4-methyl)-10,13-dioxo-2,3,9,10,13,15-hexahydro-1H,12H-benzo-[de]pyrano[3′,4′:6,7]indolizino[1,2-b]quinolin-1-yl)amino)-2-oxoethoxy)methyl)amino)-2-oxoethyl)-3-phenylpropanamide (113.1 mg, 0.13 mmol) were dissolved in DMF (3 mL), added with 2-(7-azobenzotriazolyl)-N,N,N′,N′-tetramethyluronium hexafluorophosphate (62.7 mg, 0.16 mmol) and DIPEA (42.7 mg, 0.33 mmol). After the reaction was completed, the title compound (55.7 mg, 0.04 mmol) was obtained after purification by flash column chromatography (C18, water/acetonitrile=0.6) and freeze-drying.

Its structural characterization data are as follows:

MS m/z (ESI): 1491.3 [M+H]⁺

### Step 7: Preparation of N-((10S,19S)-23-amino-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-1,6,9,12,15,18-hexaoxo-3-oxa-5,8,11,14,17-pentaazatricosan-19-yl)-6-(2-(methylsulfonyl)pyrimidin-5-yl)hex-5-ynamide (B-8-8)

N-((7S,16S)-16-benzyl-25-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-8,11,14,17,20,25-hexaoxo-1,1-diphenyl-1-(p-tolyl)-23-oxa-2,9,12,15,18,21-hexaazapentacosan-7-yl)-6-(2-(methylsulfonyl)pyrimidin-5-yl)hex-5-ynamide (55.7 mg, 0.04 mmol) was dissolved in a mixed solvent of dichloromethane (3 mL) and methanol (1 mL), added with 3 mL of formic acid, and stirred at room temperature for 1 hour. After the reaction was completed, the product was purified by flash column chromatography (C18, water/acetonitrile=0.4) and freeze-dried to obtain the title compound (27.1 mg, 0.02 mmol).

Its structural characterization data are as follows:

MS m/z (ESI): 1235.3 [M+H]⁺

### Step 8: Preparation of 2,2′,2″-(10-((10S,19S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-19-(6-(2-(methylsulfonyl)pyrimidin-5-yl)hex-5-ynamido)-1,6,9,12,15,18,25-heptaoxo-3-oxa-5,8,11,14,17,24-hexaazabexacosan-26-yl)-1,4,7,10-tetraazacyclododecan-1,4,7-triyl)triacetic acid (B-8)

N-((10S,19S)-23-Amino-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18-hexaoxo-3-oxa-5,8,11,14,17-pentaazatricosan-19-yl)-6-(2-(methylsulfonyl)pyrimidin-5-yl)hex-5-ynamide (27.1 mg, 0.02 mmol) was dissolved in DMF (2 mL), added with DIPEA (12.9 mg, 0.1 mmol), and added with 2,2′,2″-(10-(2-((2,5-dioxopyrrolidin-1-yl) oxy)-2-oxoethyl)-1,4,7,10-tetraazacyclododecan-1,4,7-triyl)triacetic acid (30.1 mg, 0.06 mmol). After the reaction was completed, the reaction solution was directly purified by preparative high performance liquid chromatography and freeze-dried to obtain the title compound (11.9 mg, 7.3 μmol).

Its structural characterization data are as follows:

MS m/z (ESI): 1622.1 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 2.00 | 10 | 90 | 30 |
| 20.00 | 90 | 10 | 30 |

### Example 28: Preparation of 2,2′-(7-((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-1,6,9,12,15,18-hexaoxo-3-oxa-5,8,11,14,17-pentaazanonadecan-19-yl)-10-(6-(2-(methylsulfonyl)pyrimidin-5-yl)hex-5-ynoyl)-1,4,7,10-tetraazacyclododecan-1,4-diyl)diacetic acid (B-9)

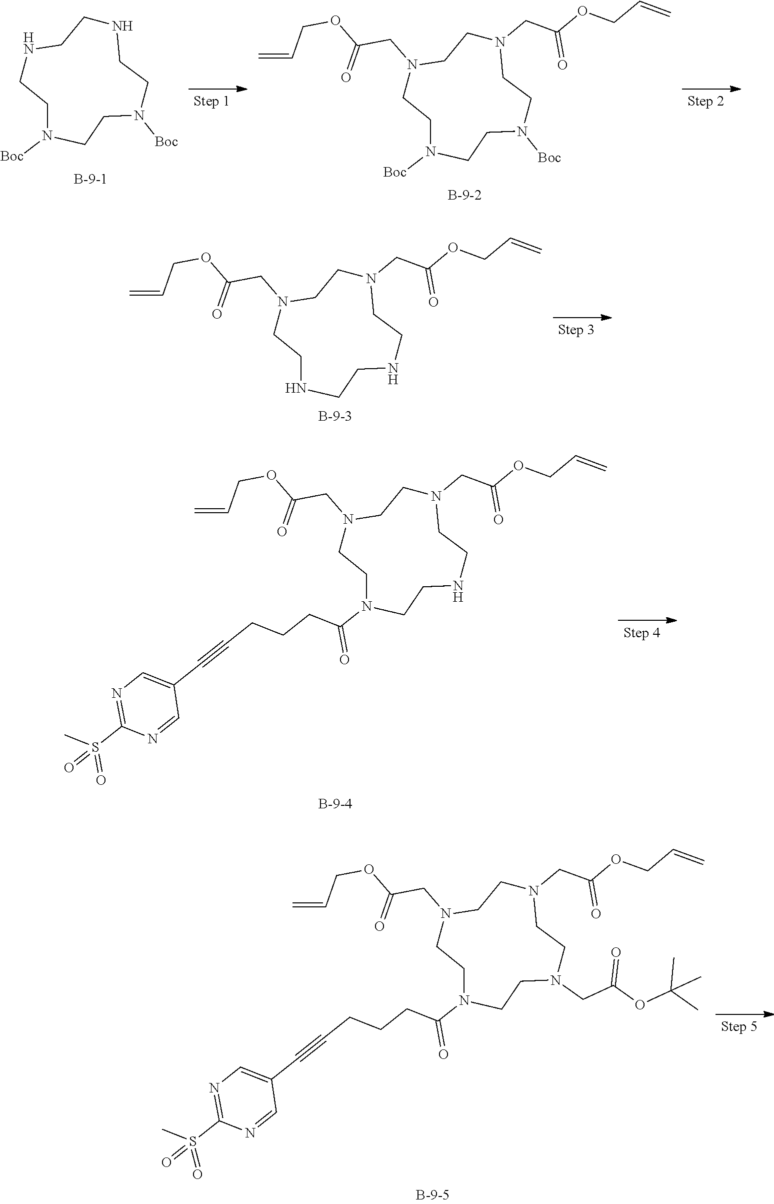

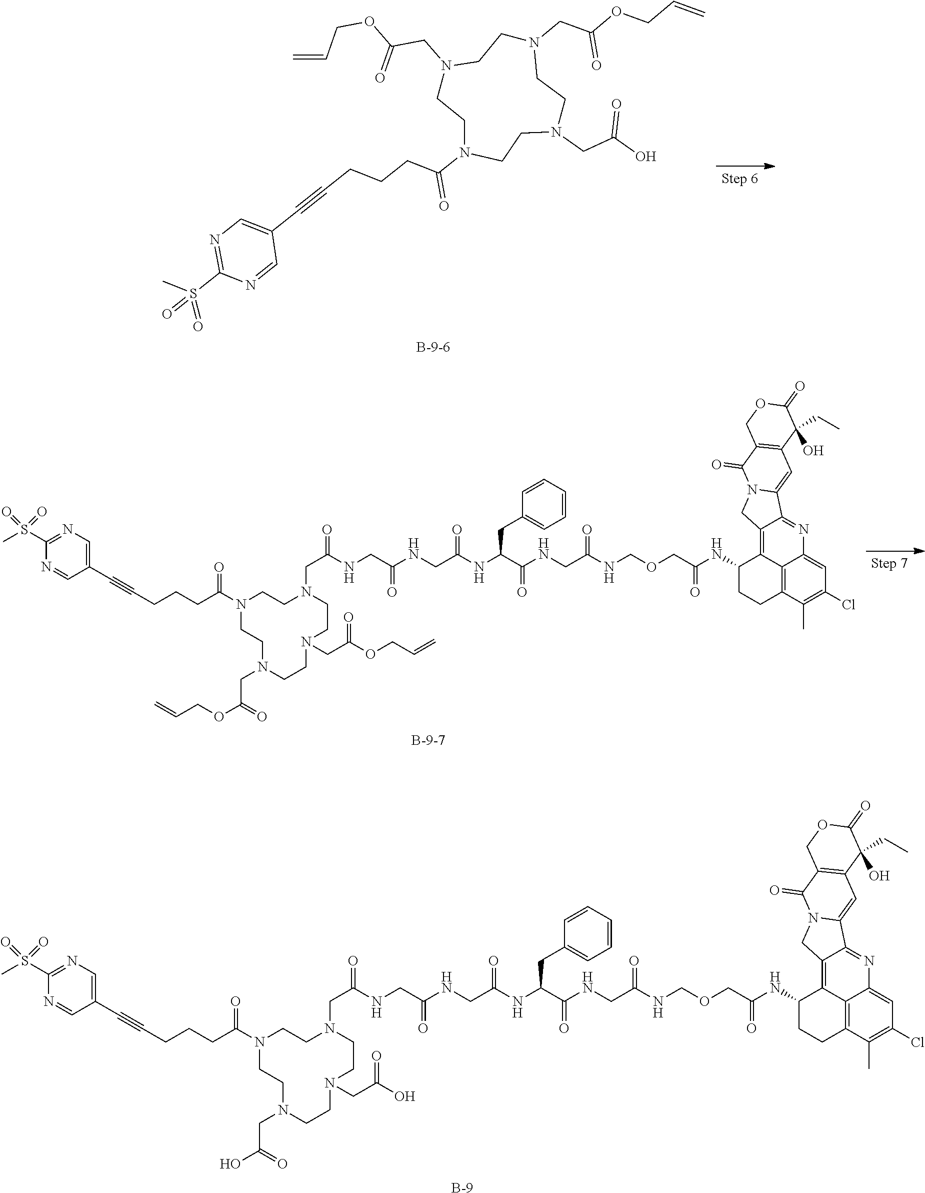

### Step 1: Preparation of di-tert-butyl 7,10-bis(2-(allyloxy)-2-oxoethyl)-1,4,7,10-tetraazacyclododecan-1,4-dicarboxylate (B3-9-2)

Di-tert-butyl 1,4,7,10-tetraazacyclododecan-1,4-dicarboxylate (0.5 g, 1.34 mmol) was dissolved in DMF (5 mL), added with allyl 2-chloroacetate (541.85 mg, 4.03 mmol) and potassium carbonate (556.53 mg, 4.03 mmol). After the addition, the system was heated to 70° C. and stirred for 20 hours. After the reaction was completed, the reaction solution was cooled to room temperature, and added with 20 mL of water and 10 mL×3 of ethyl acetate for extraction. The organic phases were washed with brine, dried over anhydrous sodium sulfate, and concentrated under reduced pressure to obtain the crude product of the title compound (760 mg, 1.34 mmol), which was used directly in the next step without purification.

Its structural characterization data are as follows:

MS m/z (ESI): 569.4 [M+H]⁺

### Step 2: Preparation of diallyl 2,2′-(1,4,7,10-tetraazacyclododecan-1,4-diyl)diacetate (B-9-3)

Ethyl acetate (5 mL) was added to di-tert-butyl 7,10-bis(2-(allyloxy)-2-oxoethyl)-1,4,7,10-tetraazacyclododecan-1,4-dicarboxylate (760 mg, 1.34 mmol), then added with a dioxane solution of HCl (4 M, 4 mL), and stirred at room temperature for 2 hours. After the reaction was completed, the reaction solution was filtered, the filter cake was washed with methyl tert-butyl ether, and then the filter cake was dried under reduced pressure to obtain the hydrochloride of the title compound (530 mg, 1.20 mmol).

Its structural characterization data are as follows:

MS m/z (ESI): 369.3 [M+H]⁺

### Step 3: Preparation of diallyl 2,2′-(7-(6-(2-(methylsulfonyl)pyrimidin-5-yl)hex-5-ynoyl)-1,4,7,10-tetraazacyclododecan-1,4-diyl)diacetate (B-9-4)

6-(2-(Methylsulfonyl)pyrimidin-5-yl)hex-5-ynoic acid (293.09 mg, 1.09 mmol) and diallyl 2,2′-(1,4,7,10-tetraazacyclododecan-1,4-diyl)diacetate (480 mg, 1.09 mmol, hydrochloride) were dissolved in DMF (5 mL), added with DIPEA (564.76 mg, 4.37 mmol) and HATU (498.46 mg, 1.31 mmol), and reacted under stirring at room temperature for 5 hours. After the reaction was completed, the reaction solution was added with water and ethyl acetate for extraction, the organic phase was washed with brine, dried over anhydrous sodium sulfate, and concentrated under reduced pressure to obtain the crude product of the title compound (500 mg, 808.09 μmol), which was directly used in the next step without purification.

Its structural characterization data are as follows:

MS m/z (ESI): 619.3 [M+H]⁺

### Step 4: Preparation of diallyl 2,2′-(7-(6-(2-(methylsulfonyl)pyrimidin-5-yl)hex-5-ynoyl)-1,4,7,10-tetraazacyclododecan-1,4-diyl)diacetate (B-9-5)

The crude product of diallyl 2,2′-(7-(6-(2-(methylsulfonyl)pyrimidin-5-yl)hex-5-ynoyl)-1,4,7,10-tetraazacyclododecan-1,4-diyl)diacetate (500 mg, 808.09 μmol) and tert-butyl 2-bromoacetate (157.62 mg, 808.09 mol) were dissolved in DMF (5 mL), added with DIPEA (208.88 mg, 1.62 mmol), heated to 50° C. and reacted under stirring for 1.5 hours. After the reaction was completed, the reaction solution added with water and ethyl acetate for extraction, and the organic phase was washed with brine, dried over anhydrous sodium sulfate and concentrated under reduced pressure to obtain a crude product, which was then purified by silica gel column chromatography (dichloromethane/(dichloromethane:methanol=3:1)=0 to 50%) and concentrated under reduced pressure again to obtain the title compound (300 mg, 409.34 μmol).

Its structural characterization data are as follows:

MS m/z (ESI): 733.4 [M+H]⁺

### Step 5: Preparation of 2-(4,7-bis(2-(allyloxy)-2-oxoethyl)-10-(6-(2-(methylsulfonyl) pyrimidin-5-yl)hex-5-ynoyl)-1,4,7,10-tetraazacyclododecan-1-yl)acetic acid (B-9-6)

Diallyl 2,2′-(7-(6-(2-(methylsulfonyl)pyrimidin-5-yl)hex-5-ynoyl)-1,4,7,10-tetraaza-cyclododecan-1,4-diyl)diacetate (300 mg, 409.34 μmol) was dissolved in dichloromethane (1.5 mL), added with TFA (3 mL), and reacted under stirring at room temperature for 24 hours. After the reaction was completed, the reaction solution was concentrated under reduced pressure to obtain a crude product, which was purified by preparative high performance liquid chromatography and freeze-dried to obtain the trifluoroacetate salt of the title compound (85 mg, 107.49 μmol).

Its structural characterization data are as follows:

MS m/z (ESI): 677.3 [M+H]⁺

Its preparation method was as follows:

Chromatographic column: Waters Sunfire Prep C18
OBD (5 μm*19 mm*150 mm)
Mobile phase A: acetonitrile; Mobile phase B: water (0.05%
trifluoroacetic acid)

| | | | Flow rate |
|---|---|---|---|
| Time [min] | Mobile phase A [%] | Mobile phase B [%] | [mL/min] |
| 0.00 | 25 | 75 | 30 |
| 2.00 | 25 | 75 | 30 |
| 20.00 | 70 | 30 | 30 |

### Step 6: Preparation of diallyl 2,2′-(7-((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano-[3′,4′:6,7]indolizino[1,2-b]quinolin-1-yl)amino)-1,6,9,12,15,18-hexaoxo-3-oxa-5,8,11,14,17-pentaazanonadecan-19-yl)-10-(6-(2-(methylsulfonyl)pyrimidin-5-yl)hex-5-ynoyl)-1,4,7,10-tetraazacyclododecan-1,4-diyl)diacetate (B-9-7)

(S)-2-(2-(2-Aminoacetamido)acetamido)-N-(2-(((2-(((1S,9S)-5-chloro-9-ethyl-9-hydroxy-4-methyl)-10,13-dioxo-2,3,9,10,13,15-hexahydro-1H,12H-benzo[de]pyrano[3′,4′:6,7]-indolizino[1,2-b]quinolin-1-yl)amino)-2-oxoethoxy)methyl)amino)-2-oxoethyl)-3-phenylpropanamide (5 mg, 5.83 μmol) and 2-(4,7-bis(2-(allyloxy)-2-oxoethyl)-10-(6-(2-(methylsulfonyl)pyrimidin-5-yl) hex-5-ynoyl)-1,4,7,10-tetraazacyclododecane-1-yl)acetic acid (4.61 mg, 5.83 μmol, trifluoroacetic acid) were dissolved in DMF (1 mL), added with DIPEA (4 mg, 30.95 μmol), then added with HATU (2.66 mg, 7.00 μmol), and reacted under stirring at room temperature for 1 hour. After the reaction was completed, the reaction solution was directly purified by preparative high performance liquid chromatography and freeze-dried to obtain the title compound (4 mg, 2.64 μmol).

Its structural characterization data are as follows:

MS m/z (ESI): 1515.7 [M+H]⁺

Its preparation method was as follows:

Chromatographic column: Waters Sunfire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 2.00 | 10 | 90 | 30 |
| 20.00 | 90 | 10 | 30 |

### Step 7: Preparation of 2,2′-(7-((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]-indolizino[1,2-b]quinolin-1-yl)amino)-1,6,9,12,15,18-hexaoxo-3-oxa-5,8,11,14,17-pentaazanonadecan-19-yl)-10-(6-(2-(methylsulfonyl)pyrimidin-5-yl)hex-5-ynoyl)-1,4,7,10-tetraazacyclododecan-1,4-diyl)diacetic acid (B-9)

Diallyl 2,2′-(7-((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18-hexaoxo-3-oxa-5,8,11,14,17-pentaazanonadecan-19-yl)-10-(6-(2-(methylsulfonyl)pyrimidin-5-yl)hex-5-ynoyl)-1,4,7,10-tetraazacyclododecan-1,4-diyl)diacetate (4.00 mg, 2.64 μmol, FR) was dissolved in DMF (1 mL), added with formic acid (2.64 μmol, 10 μL) and N-methylmorpholine (2.64 μmol, 20 μL), added with tetratriphenylphosphine palladium (3.05 mg, 2.64 μmol), subjected to nitrogen replacement three times, and then reacted under stirring at room temperature for 1 hour. After the reaction was completed, the reaction solution was directly purified by preparative high performance liquid chromatography and freeze-dried to obtain the title compound (1.74 mg, 1.15 μmol).

Its structural characterization data are as follows:

MS m/z (ESI): 1035.5 [M+H]⁺

Its preparation method was as follows:

Chromatographic column: Waters Sunfire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 32 |
| 4.00 | 20 | 80 | 32 |
| 24.00 | 90 | 10 | 32 |

### Example 29: Preparation of 2,2′-(4-(2-((2-((2-(((S)-1-((2-(((2-(((1S,9S)-5-chloro-9-ethyl-9-hydroxy-4-methyl-10,13-dioxo-2,3,9,10,13,15-hexahydro-1H,12H-benzo[de]pyrano-[3′,4′:6,7]indolizino[1,2-b]quinolin-1-yl)amino)-2-oxoethoxy)methyl)amino)-2-oxoethyl)-amino)-1-oxo-3-phenylpropan-2-yl)amino)-2-oxoethyl)amino)-2-oxoethyl)amino)-2-oxoethyl)-10-((6-(2-(methylsulfonyl)pyrimidin-5-yl)hex-5-ynoyl)glycyl)-1,4,7,10-tetraazacyclododecan-1,7-diyl)diacetic acid (B-11)

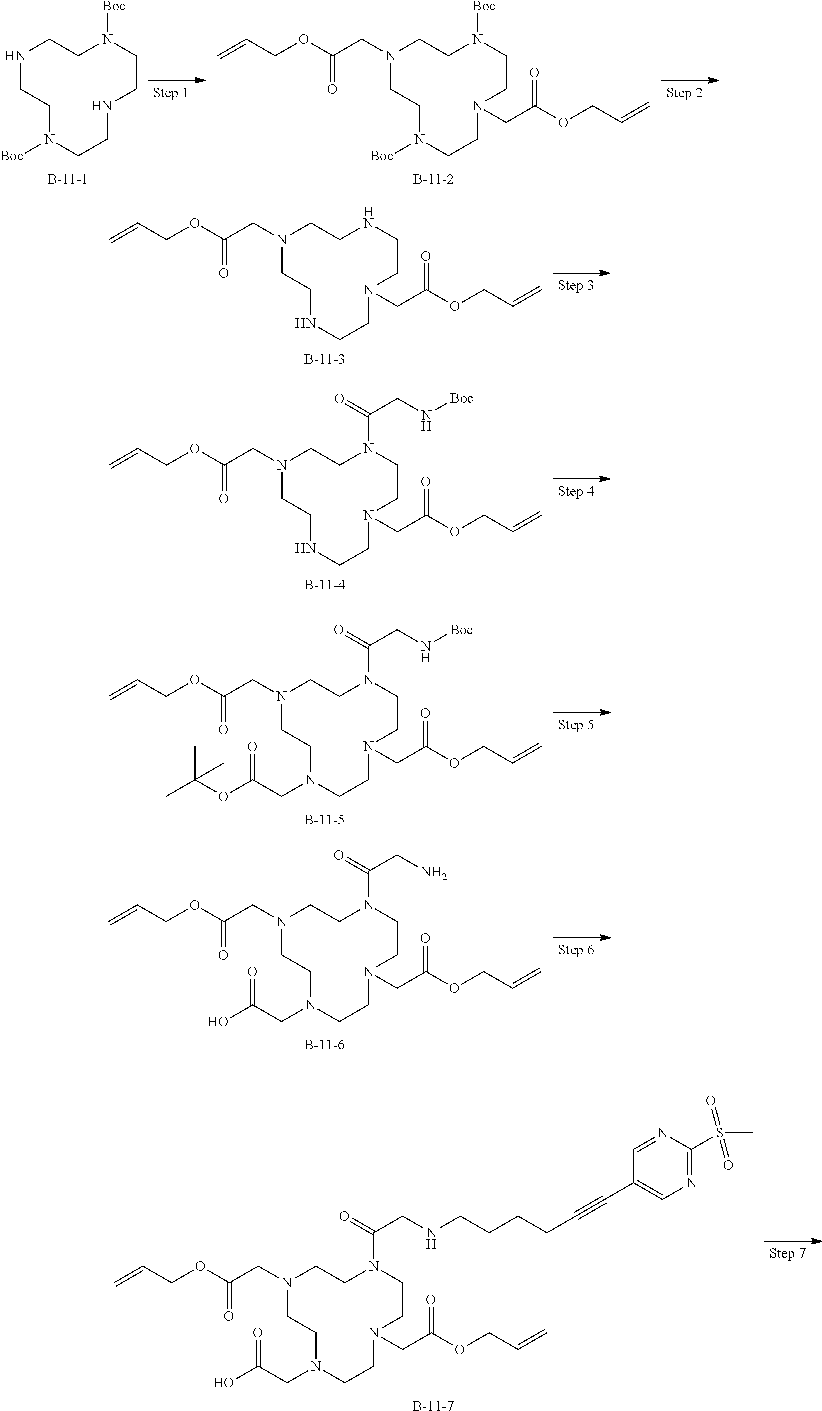

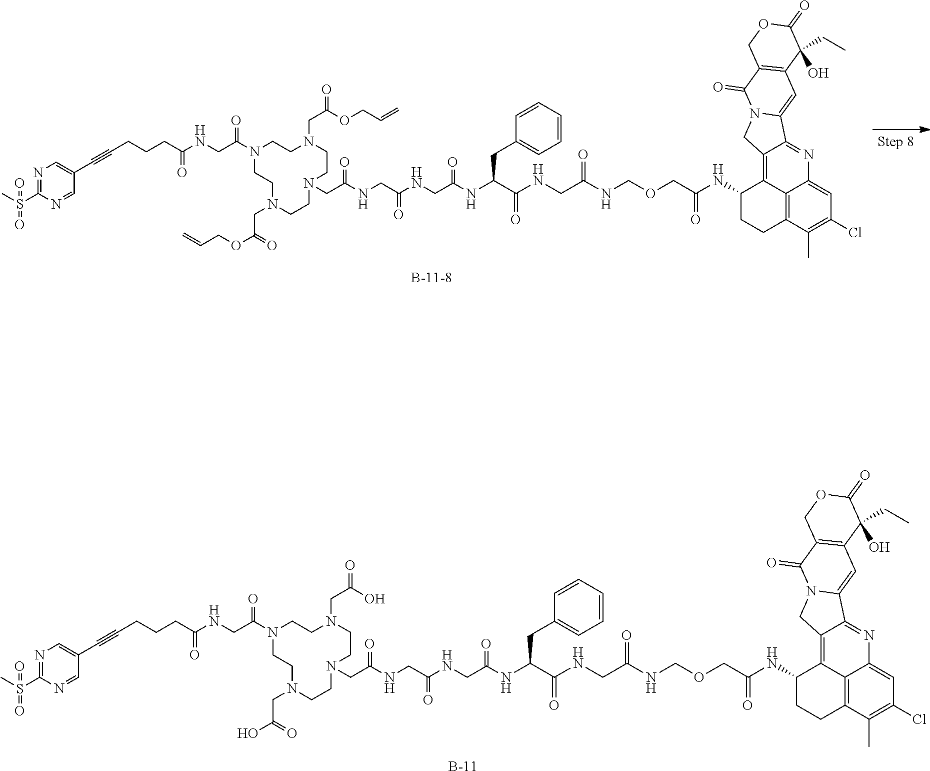

### Step 1: Preparation of di-tert-butyl 4,10-bis(2-(allyloxy)-2-oxoethyl)-1,4,7,10-tetraazacyclododecan-1,7-dicarboxylate (B-111-2)

Di-tert-butyl 1,4,7,10-tetraazacyclododecan-1,7-dicarboxylate (1.0 g, 2.69 mmol) was dissolved in dichloromethane (10 mL), added with DIPEA (3.5 g, 26.9 mmol), added with allyl chloroacetate (18.1 g, 134.5 mmol), and stirred at room temperature under nitrogen protection for 72 hours. After the reaction was completed, the reaction solution was concentrated under reduced pressure to obtain a crude product, which was purified by preparative high performance liquid chromatography and freeze-dried to obtain the title compound (1.1 g, 1.9 mmol).

Its structural characterization data are as follows:

MS m/z (ESI): 569.1 [M+H]⁺

### Step 2: Preparation of diallyl 2,2′-(1,4,7,10-tetraazacyclododecan-1,7-diyl)diacetate (B-11-3)

Di-tert-butyl 4,10-bis(2-(allyloxy)-2-oxoethyl)-1,4,7,10-tetraazacyclododecan-1,7-dicarboxylate (1.1 g, 1.9 mmol) was dissolved in dichloromethane (15 mL), added with trifluoroacetic acid (10 mL), and stirred at room temperature for 1 hour. After the reaction was completed, the solvent was evaporated off to obtain the title compound as crude product (690.0 mg, 1.87 mmol).

Its structural characterization data are as follows:

MS m/z (ESI): 369.1 [M+H]⁺

### Step 3: Preparation of diallyl 2,2′-(4-((tert-butoxycarbonyl)glycyl)-1,4,7,10-tetraazacyclododecan-1,7-diyl)diacetate (B-11-4)

Diallyl 2,2′-(1,4,7,10-tetraazacyclododecan-1,7-diyl)diacetate (690.0 mg, 1.87 mmol) was dissolved in DMF (8 mL), added with DIPEA (723.7 mg, 5.61 mmol), and added with 2,5-dioxopyrrolidin-1-yl (tert-butoxycarbonyl)glycinate (483.2 mg, 1.77 mmol). After the reaction was completed, the reaction solution was directly purified by preparative high performance liquid chromatography and freeze-dried to obtain the title compound (760.2 mg, 1.45 mmol).

Its structural characterization data are as follows:

MS m/z (ESI): 526.2 [M+H]⁺

### Step 4: Preparation of diallyl 2,2′-(4-(2-(tert-butoxy)-2-oxoethyl)-10-((tert-butoxycarbonyl) glycyl)-1,4,7,10-tetraazacyclododecan-1,7-diyl)diacetate (B-11-5)

Diallyl 2,2′-(4-((tert-butoxycarbonyl)glycyl)-1,4,7,10-tetraazacyclododecan-1,7-diyl) diacetate (760.2 mg, 1.45 mmol) was dissolved in DMF (6 mL), added with DIPEA (935.3 mg, 7.25 mmol), added with tert-butyl bromoacetate (848.2 mg, 4.35 mmol), and stirred at 50° C. for 2 hours under nitrogen protection. After the reaction was completed, the reaction solution was directly purified by preparative high performance liquid chromatography and freeze-dried to obtain the title compound (556.6 mg, 0.87 mmol).

Its structural characterization data are as follows:

MS m/z (ESI): 640.2 [M+H]⁺

### Step 5: Preparation of 2-(4,10-bis(2-(allyloxy)-2-oxoethyl)-7-glycyl-1,4,7,10-tetraazacyclododecan-1-yl)acetic acid (B-11-6)

Diallyl 2,2′-(4-(2-(tert-butoxy)-2-oxoethyl)-10-((tert-butoxycarbonyl)glycyl)-1,4,7,10-tetraazacyclododecan-1,7-diyl)diacetate (556.6 mg, 0.87 mmol) was dissolved in dichloromethane (10 mL), added with trifluoroacetic acid (6 mL), and stirred at room temperature for 15 hours. After the reaction was completed, the solvent was evaporated off to obtain the title compound as crude product (378.2 mg, 0.78 mmol).

Its structural characterization data are as follows:

MS m/z (ESI): 484.2 [M+H]⁺

### Step 6: Preparation of 2-(4,10-bis(2-(allyloxy)-2-oxoethyl)-7-((6-(2-(methylsulfonyl) pyrimidin-5-yl)hex-5-ynoyl)glycyl)-1,4,7,10-tetraazacyclododecan-1-yl)acetic acid (B-11-7)

2-(4,10-Bis(2-(allyloxy)-2-oxoethyl)-7-glycyl-1,4,7,10-tetraazacyclododecan-1-yl)acetic acid (378.2 mg, 0.78 mmol) was dissolved in DMF (5 mL), added with DIPEA (301.8 mg, 2.34 mmol), added with 2,5-dioxopyrrolidin-1-yl 6-(2-(methylsulfonyl)pyrimidin-5-yl)hex-5-ynoate (569.4 mg, 1.56 mmol), and stirred at room temperature for 2 hours. After the reaction was completed, the reaction solution was directly purified by preparative high performance liquid chromatography and freeze-dried to obtain the title compound (343.1 mg, 0.47 mmol).

Its structural characterization data are as follows:

MS m/z (ESI): 734.2 [M+H]⁺

### Step 7: Preparation of diallyl 2,2′-(4-(2-((2-((2-(((S)-1-((2-(((2-(((1S,9S)-5-chloro-9-ethyl-9-hydroxy-4-methyl-10,13-dioxo-2,3,9,10,13,15-hexahydro-1H,12H-benzo[de]pyrano-[3′,4′:6,7]indolizino [1,2-b]quinolin-1-yl)amino)-2-oxoethoxy)methyl)amino)-2-oxoethyl)amino)-1-oxo-3-phenylpropan-2-yl)amino)-2-oxoethyl)amino)-2-oxoethyl)amino-2-oxoethyl)-10-((6-(2-methylsulfonyl)pyrimidin-5-yl)hex-5-ynoyl)glycyl)-1,4,7,10-tetraazacyclododecan-1,7-diyl)diacetate (B-11-8)

2-(4,10-Bis(2-(allyloxy)-2-oxoethyl)-7-((6-(2-(methylsulfonyl)pyrimidin-5-yl)hex-5-ynoyl)glycyl)-1,4,7,10-tetraazacyclododecan-1-yl)acetic acid (100.0 mg, 0.14 mmol), (S)-2-(2-(2-aminoacetamido)acetamido)-N-(2-(((2-(((1S,9S)-5-chloro-9-ethyl-9-hydroxy-4-methyl)-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)-3-phenylpropanamide (116.9 mg, 0.14 mmol) were dissolved in DMF (3 mL), added with 2-(7-azobenzotriazole)-N,N,N′,N′-tetramethyluronium hexafluorophosphate (62.7 mg, 0.16 mmol) and DIPEA (58.1 mg, 0.45 mmol). After the reaction was completed, the title compound (88.1 mg, 0.05 mmol) was obtained after purification by flash column chromatography (C18, water/acetonitrile=0.6) and freeze-drying.

Its structural characterization data are as follows:

MS m/z (ESI): 1572.3 [M+H]⁺

### Step 8: Preparation of 2,2′-(4-(2-((2-((2-(((S)-1-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)-amino)-2-oxoethyl)amino)-1-oxo-3-phenylpropan-2-yl)amino)-2-oxoethyl)amino)-2-oxoethyl)amino)-2-oxoethyl)-10-((6-(2-(methylsulfonyl)pyrimidin-5-yl)hex-5-ynoyl)glycyl)-1,4,7,10-tetraazacyclododecan-1,7-diyl)diacetic acid (B-11)

Diallyl 2,2′-(4-(2-((2-((2-(((S)-1-((2-(((2-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-1-oxo-3-phenylpropan-2-yl)amino)-2-oxoethyl)amino)-2-oxoethyl)amino)-2-oxoethyl)-10-((6-(2-(methylsulfonyl)pyrimidin-5-yl)hex-5-ynoyl)glycyl)-1,4,7,10-tetraazacyclododecan-1,7-diyl)diacetate (88.1 mg, 0.05 mmol) was dissolved in DMF (5 mL), added with tetratriphenylphosphine palladium (11.6 mg, 0.1 mmol), added with 50 μL of formic acid and 100 μL of N-methylmorpholine, and stirred at room temperature under nitrogen protection for 2 hours. After the reaction was completed, the reaction solution was directly purified by preparative high performance liquid chromatography and freeze-dried to obtain the title compound (1.97 mg, 1.3 μmol).

Its structural characterization data are as follows:

MS m/z (ESI): 1493.1 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 20 | 80 | 32 |
| 4.00 | 20 | 80 | 32 |
| 24.00 | 90 | 10 | 32 |

### Example 30: Preparation of N-((4S,9S,12S,15S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-4-cyclopropyl-9,12-dimethyl-3,8,11,14-tetraoxo-5-oxa-2,7,10,13-tetraazahexadecan-15-yl)-6-(2-(methylsulfonyl)pyrimidin-5-yl)hex-5-ynamide (A-94)

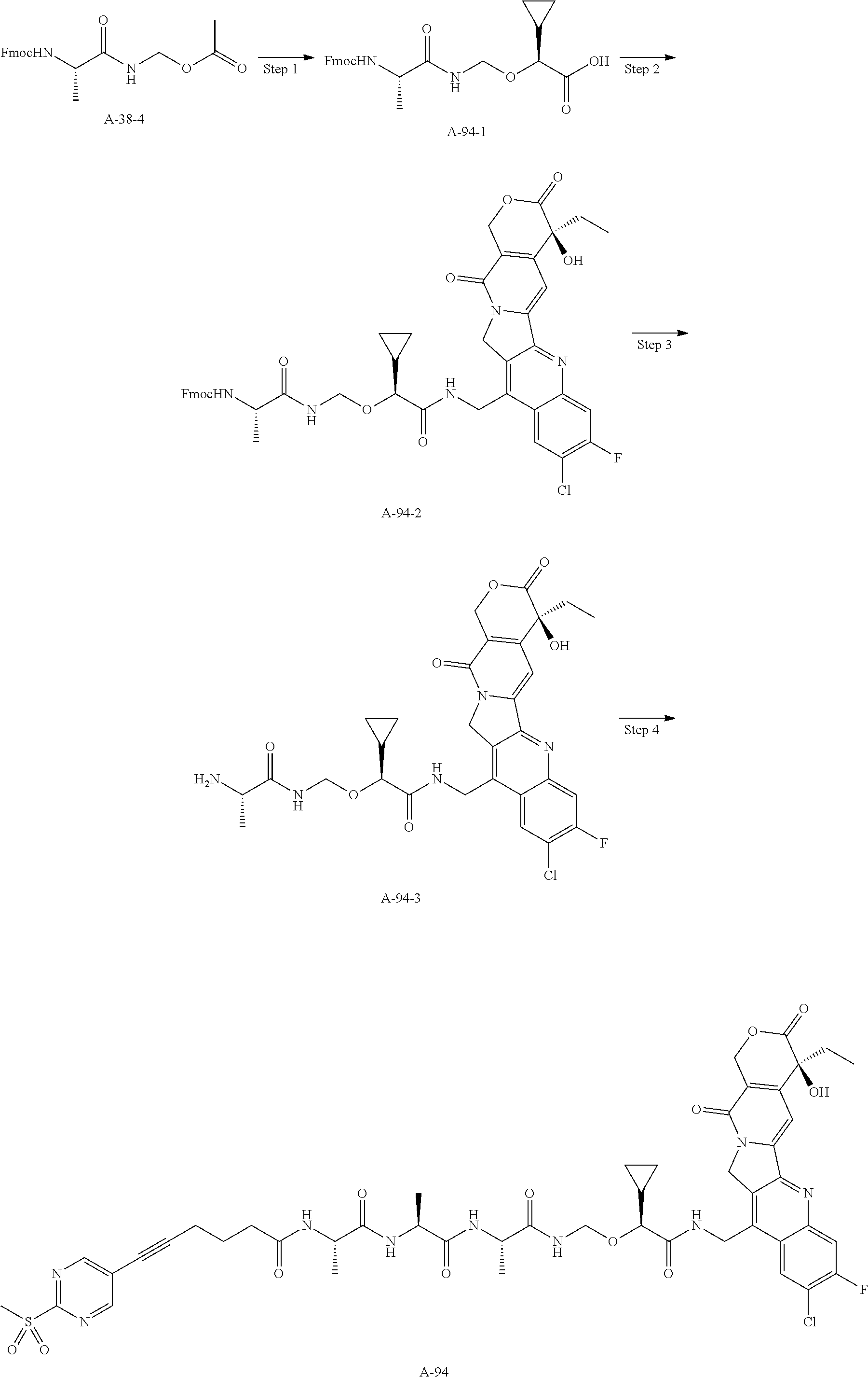

### Step 1: Preparation of (5S,10S)-10-cyclopropyl-1-(9H-fluoren-9-yl)-5-methyl-3,6-dioxo-2,9-dioxa-4,7-diazaundecane-11-oleic acid (A-94-1)

Methyl (S)-(2-((((9H-fluoren-9-yl)methoxy)carbonyl)amino)propionamido)acetate (1.5 g, 3.92 mmol) and (S)-2-cyclopropyl-2-hydroxyacetic acid (1.37 g, 11.77 mmol) were dissolved in acetonitrile (100 mL), added with p-toluenesulfonic acid (202.64 mg, 1.18 mmol), and reacted at 20° C. for 1 hour. After the reaction was completed, the reaction solution was diluted with water and extracted with ethyl acetate. The organic phase was dried and concentrated to obtain a pale yellow viscous crude product, which was purified by silica gel column chromatography to obtain the white title compound (190 mg, 433.32 μmol).

Its structural characterization data are as follows:

MS m/z (ESI): 461.3 [M+Na]⁺

### Step 2: Preparation of (9H-fluoren-9-yl)methyl ((S)-1-((((S)-2-((((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)amino)-1-cyclopropyl-2-oxoethoxy)methyl)amino)-1-oxopropan-2-yl)-carbamate (A-94-2)

(5S,10S)-10-Cyclopropyl-1-(9H-fluoren-9-yl)-5-methyl-3,6-dioxo-2,9-dioxa-4,7-diazaundecan-11-oleic acid (100 mg, 182.45 μmol) was dissolved in N,N-dimethylformamide (100 mL), and added with HATU (104.06 mg, 273.68 μmol) and N,N-diisopropylethylamine (70.74 mg, 547.36 μmol). The reaction was carried out under stirring for 30 minutes, and (S)-11-(aminomethyl)-9-chloro-4-ethyl-8-fluoro-4-hydroxy-1,12-dihydro-14H-pyrano[3′,4′:6,7]indolizino[1,2-b]quinolin-3,14(4H)-dione (110.25 mg, 182.45 μmol) was added.

The reaction was allowed to react at 25° C. for 2 hours. After the reaction was completed, the reaction solution was purified by preparative HPLC to obtain the white title compound (80 mg, 89.38 μmol).

Its structural characterization data are as follows:

MS m/z (ESI): 850.1 [M+H]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 50 | 50 | 30 |
| 2.00 | 50 | 50 | 30 |
| 15.00 | 70 | 30 | 30 |

### Step 3: Preparation of (S)-2-amino-N—(((S)-2-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)amino)-1-cyclopropyl-2-oxoethoxy)methyl)propanamide (A-94-3)

(9H-Fluoren-9-yl)methyl ((S)-1-((((S)-2-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-amino)-1-cyclopropyl-2-oxoethoxy)methyl)amino)-1-oxopropan-2-yl)carbamate (80 mg, 94.09 μmol) was dissolved in N,N-dimethylformamide (5 mL), and added with diethylamine (13.76 mg, 188.17 μmol). The reaction solution was purified by preparative HPLC to obtain the title compound (55 mg, 83.19 μmol).

Its structural characterization data are as follows:

MS m/z (ESI): 629.3 [M+H]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 10 | 90 | 30 |
| 4.00 | 10 | 90 | 30 |
| 24.00 | 90 | 10 | 30 |

### Step 4: Preparation of N-((4S,9S,12S,15S)-1-((S)-9-chloro-4-ethyl-8-fluoro-4-hydroxy-3,14-dioxo-3,4,12,14-tetrahydro-TH-pyrano[3′,4′:6,7]indolizino[1,2-b]quinolin-11-yl)-4-cyclopropyl-9,12-dimethyl-3,8,11,14-tetraoxo-5-oxa-2,7,10,13-tetraazahexadecan-15-yl)-6-(2-(methylsulfonyl)pyrimidin-5-yl)hex-5-ynamide (A-94)

(S)-2-Amino-N—(((S)-2-((((S)-9-chloro-4-ethyl-8-fluoro-4-hydroxy-3,14-dioxo-3,4,12,14-tetrahydro-TH-pyrano[3′,4′:6,7]indolizino[1,2-b]quinolin-11-yl)methyl)amino)-1-cyclopropyl-2-oxoethoxy)methyl)propanamide (20 mg, 29.67 μmol) and (6-(2-(methylsulfonyl)pyrimidin-5-yl) hex-5-ynoyl)-L-alanyl-L-alanine (13.40 mg, 32.64 μmol) were dissolved in N,N-dimethylformamide (2 mL), added with N,N-diisopropylethylamine (11.50 mg, 89.01 μmol) and HATU (22.56 mg, 59.34 μmol), and reacted at 25° C. for 1 hour. After the reaction, the reaction solution was purified to obtain the title compound (10.67 mg, 10.56 μmol).

Its structural characterization data are as follows:

MS m/z (ESI): 1020.4 [M+H]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 30 |
| 2.00 | 30 | 70 | 30 |
| 18.00 | 90 | 10 | 30 |

### Example 31: Preparation of N-((4S,9S,12S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-4-cyclopropyl-12-isopropyl-9-methyl-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-95)

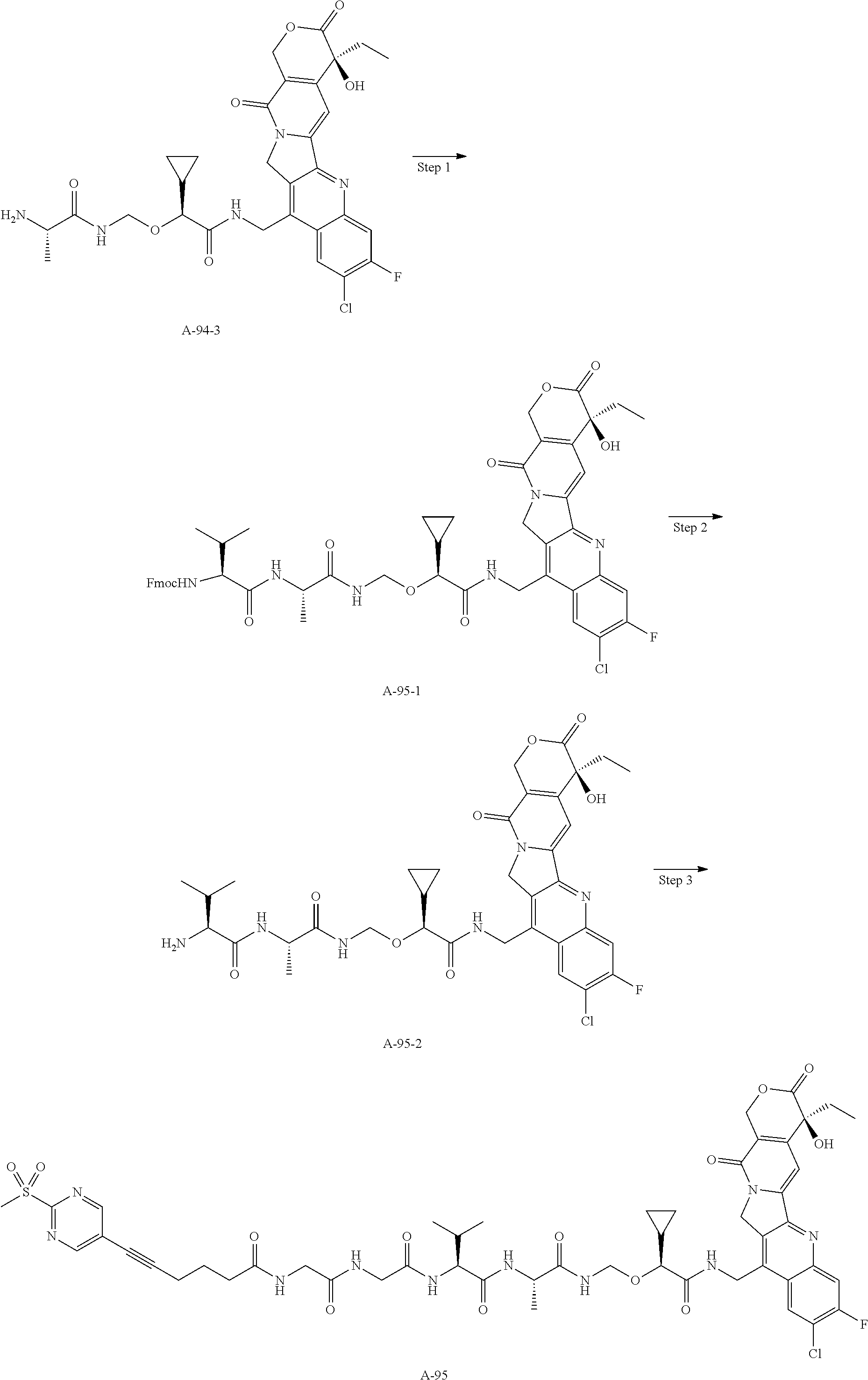

### Step 1: Preparation of (9H-fluoren-9-yl)methyl ((4S,9S,12S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-4-cyclopropyl-9,13-dimethyl-3,8,11-trioxo-5-oxa-2,7,10-triazatetradecan-12-yl)carbamate (A-95-1)

(S)-2-Amino-N—(((S)-2-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)amino)-1-cyclopropyl-2-oxoethoxy)methyl)propanamide (40 mg, 59.34 μmol) and 2,5-dioxocyclopentyl (((9H-fluoren-9-yl)methoxy)carbonyl)-L-valinate (33.52 mg, 65.27 μmol) were dissolved in N,N-dimethylformamide (2 mL), then N,N-diisopropylethylamine (15.34 mg, 118.68 μmol) was added to the reaction system. The reaction was carried out under stirring at room temperature for 2 hours. After the reaction was completed, the reaction solution was purified to obtain the title compound (20 mg, 18.96 μmol).

Its structural characterization data are as follows:

MS m/z (ESI): 949.3 [M+H]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 50 | 50 | 30 |
| 2.00 | 50 | 50 | 30 |
| 15.00 | 70 | 30 | 30 |

### Step 2: Preparation of (S)-2-amino-N—((S)-1-((((S)-2-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)amino)-1-cyclopropyl-2-oxoethoxy)methyl)amino)-1-oxopropan-2-yl)-3-methylbutanamide (A-95-2)

(9H-Fluoren-9-yl)methyl ((4S,9S,12S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-4-cyclopropyl-9,13-dimethyl-3,8,11-trioxo-5-oxa-2,7,10-triazatetradecan-12-yl)carbamate (30 mg, 31.60 μmol) was dissolved in N,N-dimethylformamide (2 mL), and ethylenediamine (4.62 mg, 63.20 μmol) was added to the reaction system. The reaction was carried out under stirring at room temperature for 1 hour. After the reaction was completed, the reaction solution was purified to obtain the title compound (10 mg, 12.38 μmol).

Its structural characterization data are as follows:

MS m/z (ESI): 727.3 [M+H]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 5 | 95 | 30 |
| 2.00 | 5 | 95 | 30 |
| 20.00 | 95 | 5 | 30 |

### Step 3: Preparation of N-((4S,9S,12S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-4-cyclopropyl-12-isopropyl-9-methyl-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-95)

(6-(2-(Methylsulfonyl)pyrimidin-5-yl)hex-5-ynoyl)glycylglycine (13.64 mg, 35.66 μmol) and HATU (22.60 mg, 59.44 μmol) were dissolved in N,N-dimethylformamide (2 mL), and added with N,N-diisopropylethylamine (7.68 mg, 59.44 μmol). The reaction was carried out under stirring for 30 minutes, and then (S)-2-amino-N—((S)-1-((((S)-2-((((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)amino)-1-cyclopropyl-2-oxoethoxy)methyl)amino)-1-oxopropan-2-yl)-3-methylbutanamide (22.98 mg, 29.72 μmol). The reaction was continued under stirring at the same temperature for 2 hours. After the reaction was completed, the reaction solution was purified to obtain the title compound (3.45 mg, 3.1 μmol).

Its structural characterization data are as follows:

MS m/z (ESI): 1113.5 [M+Na]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 35 | 65 | 30 |
| 2.00 | 35 | 65 | 30 |
| 18.00 | 90 | 90 | 30 |

### Example 32: N-((4S,12S)-12-benzyl-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-4-cyclopropyl-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-96)

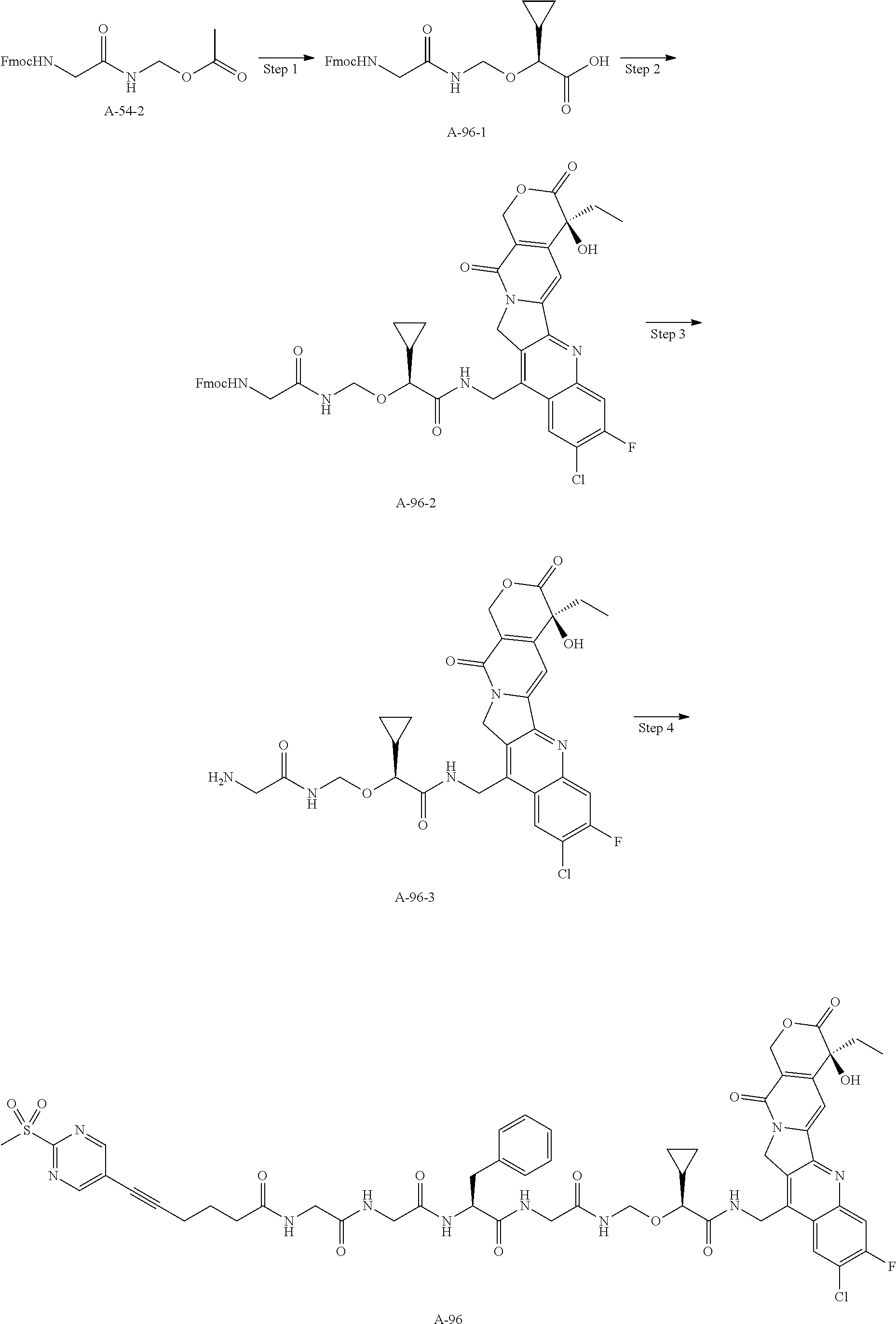

### Step 1: Preparation of (S)-10-cyclopropyl-1-(9H-fluoren-9-yl)-3,6-dioxo-2,9-dioxa-4,7-diazaundecan-11-oleic acid (A-96-1)

Methyl (2-((((9H-fluoren-9-yl)methoxy)carbonyl)amino)acetamido)acetate (300 mg, 814.37 μmol) and (S)-2-cyclopropyl-2-hydroxyacetic acid (283.68 mg, 2.44 mmol) were dissolved in tetrahydrofuran (5 mL), and p-methanesulfonic acid monohydrate (12.73 mg, 244.31 μmol) was added to the reaction system. The reaction was carried out under stirring at room temperature for 2 hours. After the reaction was completed, the reaction solution was added with water for dilution, and then extracted with ethyl acetate. The organic phase was dried and concentrated to obtain a pale yellow viscous crude product. The crude product was purified by preparative HPLC to obtain the title compound (100 mg, 212.04 μmol).

Its structural characterization data are as follows:

MS m/z (ESI): 442.1 [M+H₂O]⁺

Its preparation method was as follows:

Chromatographic column: SunFire Prep C18
OBD (5 μm*19 mm*150 mm)
Mobile phase A: acetonitrile; Mobile phase B: water (0.05%
ammonium bicarbonate)

| | | | Flow rate |
|---|---|---|---|
| Time [min] | Mobile phase A [%] | Mobile phase B [%] | [mL/min] |
| 0.00 | 20 | 80 | 30 |
| 2.00 | 20 | 80 | 30 |
| 18.00 | 70 | 30 | 30 |

### Step 2: Preparation of (9H-fluoren-9-yl)methyl (2-((((S)-2-(((((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)amino)-1-cyclopropyl-2-oxoethoxy)methyl)amino)-2-oxoethyl)carbamate (A-96-2)

(S)-10-Cyclopropyl-1-(9H-fluoren-9-yl)-3,6-dioxo-2,9-dioxa-4,7-diazaundecan-11-oleic acid (63 mg, 118.74 μmol) and HATU (90.29 mg, 237.49 μmol) were dissolved in N,N-dimethylformamide (2 mL), and N,N-diisopropylethylamine (46.04 mg, 356.23 μmol) was added to the reaction system. The reaction was carried out under stirring at room temperature for 30 minutes. Then, (S)-11-(aminomethyl)-9-chloro-4-ethyl-8-fluoro-4-hydroxy-1,12-dihydro-14H-pyrano[3′,4′:6,7]indolizino[1,2-b]quinolin-3,14(4H)-dione (71.75 mg, 118.74 μmol) was added to the reaction system. The reaction was continued for 2 hours. After the reaction was completed, the reaction solution was purified by preparation HPLC to obtain the title compound (30 mg, 32.29 μmol).

Its structural characterization data are as follows:

MS m/z (ESI): 836.3 [M+H]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 40 | 60 | 30 |
| 2.00 | 40 | 60 | 30 |
| 18.00 | 95 | 5 | 30 |

### Step 3: Preparation of (S)-2-((2-aminoacetylamino)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)-2-cyclopropylacetamide (A-96-3)

(9H-Fluoren-9-yl)methyl (2-((((S)-2-(((((S)-9-chloro-4-ethyl-8-fluoro-4-hydroxy-3,14-dioxo-3,4,12,14-tetrahydro-11H-pyrano[3′,4′:6,7]indolizino [1,2-b]quinolin-11-yl)methyl)-amino)-1-cyclopropyl-2-oxoethoxy)methyl)amino)-2-oxoethyl)carbamate (14 mg, 16.74 μmol) was dissolved in N,N-dimethylformamide (2 mL), and diethylamine (2.45 mg, 33.48 μmol) was added to the reaction system. The reaction was carried out under stirring at room temperature for 1 hour. After the reaction was completed, the reaction solution was purified by preparative HPLC to obtain the title compound (55 mg, 83.19 μmol).

Its structural characterization data are as follows:

MS m/z (ESI): 614.2 [M+H]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 40 | 60 | 30 |
| 2.00 | 40 | 60 | 30 |
| 18.00 | 95 | 5 | 30 |

### Step 4: Preparation of N-((4S,12S)-12-benzyl-1-((S)-9-chloro-4-ethyl-8-fluoro-4-hydroxy-3,14-dioxo-3,4,12,14-tetrahydro-TH-pyrano[3′,4′:6,7]indolizino[1,2-b]quinolin-11-yl)-4-cyclopropyl-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-96)

(6-(2-(Methylsulfonyl)pyrimidin-5-yl)hex-5-ynoyl)glycylglycyl-L-phenylalanine (8.02 mg, 15.15 μmol) and HATU (8.64 mg, 22.73 μmol) were dissolved in N,N-dimethylformamide (2 mL), and N,N-diisopropylethylamine (5.87 mg, 45.45 μmol) was added to the reaction system. The reaction was carried out under stirring at room temperature for 30 minutes. (S)-2-((2-aminoacetamido)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-2-cyclopropyl-acetamide (10 mg, 15.15 μmol) was added. The reaction was carried out under stirring at this temperature for 2 hours. After the reaction was completed, the reaction solution was purified to obtain the title compound (2.03 mg, 1.44 μmol).

Its structural characterization data are as follows:

MS m/z (ESI): 1126.4 [M+H]⁺

Its preparation method was as follows:

Chromatographic column: SunFire Prep C18
OBD (5 μm*19 mm*150 mm)
Mobile phase A: acetonitrile; Mobile phase B: water (0.05%
formic acid)

| | | | Flow rate |
|---|---|---|---|
| Time [min] | Mobile phase A [%] | Mobile phase B [%] | [mL/min] |
| 0.00 | 30 | 70 | 30 |
| 2.00 | 30 | 70 | 30 |
| 18.00 | 90 | 10 | 30 |

### Example 33: Preparation of N-((4R,9S,12S,15S)-4-cyclopropyl-1-((S)-4-ethyl-8-fluoro-4-hydroxy-9-methyl-3,14-dioxo-3,4,12,14-tetrahydro-TH-pyrano[3′,4′:6,7]indolizino[1,2-b]-quinolin-11-yl)-9,12-dimethyl-3,8,11,14-tetraoxo-5-oxa-2,7,10,13-tetraazahexadecan-15-yl)-6-(2-(methylsulfonyl)pyrimidin-5-yl)hex-5-ynamide (A-97)

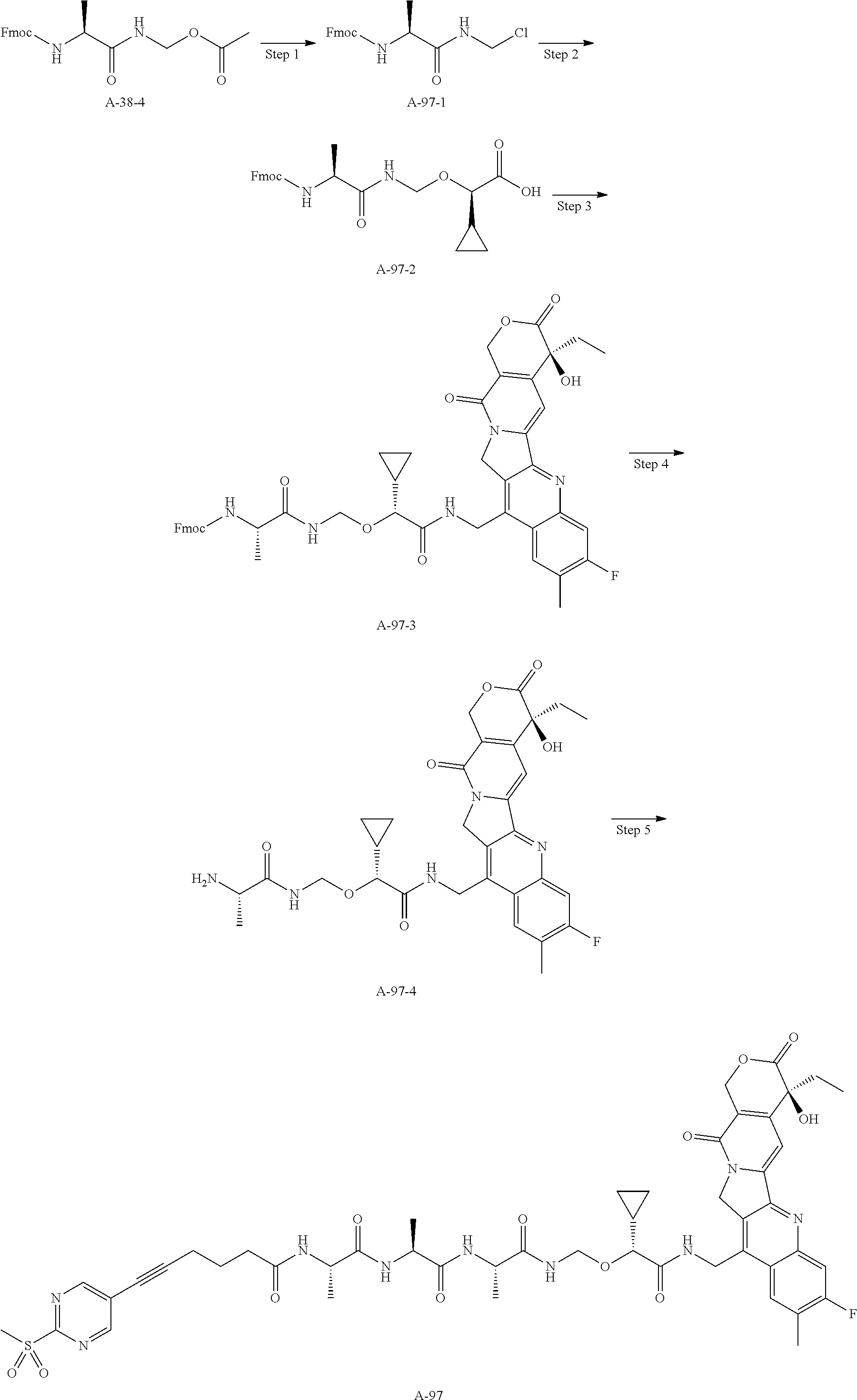

### Step 1: Preparation of (9H-fluoren-9-yl)methyl (S)-(1-((chloromethyl)amino)-1-oxopropan-2-yl)carbamate (A-97-1)

Methyl (S)-(2-((((9H-fluoren-9-yl)methoxy)carbonyl)amino)propionamido)acetate (2 g, 5.23 mmol) was dissolved in DCM (50 mL), added with TMSCl (5.68 g, 52.3 mmol), and stirred at room temperature for 1 hour. After the reaction was completed, the filtrate was collected by suction filtration and concentrated under reduced pressure to obtain the crude product of the title compound (1.88 g, 5.24 mmol), which was directly used in the next step without purification.

### Step 2: Preparation of (5S,10S)-10-cyclopropyl-1-(9H-fluoren-9-yl)-5-methyl-3,6-dioxo-2,9-dioxa-4,7-diazaundecan-11-oleic acid (A-97-2)

(9H-Fluoren-9-yl)methyl (S)-(1-((chloromethyl)amino)-1-oxopropan-2-yl)carbamate (1.88 g, 5.24 mmol) was dissolved in DCM (5 mL), added with (S)-2-cyclopropyl-2-hydroxyacetic acid (608.38 g, 5.24 mmol), and stirred at 50° C. for 1 hour. After the reaction was completed, the reaction solution was directly purified by preparative high performance liquid chromatography and freeze-dried to obtain the title compound (515.0 mg, 1.12 mmol).

Its structural characterization data are as follows:

MS m/z (ESI): 439.5 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05%
ammonium bicarbonate)

| | | | Flow rate |
|---|---|---|---|
| Time [min] | Mobile phase A [%] | Mobile phase B [%] | [mL/min] |
| 0.00 | 10 | 90 | 32 |
| 2.00 | 10 | 90 | 32 |
| 20.00 | 90 | 10 | 32 |

### Step 3: Preparation of (9H-fluoren-9-yl)methyl ((S)-1-((((R)-1-cyclopropyl-2-(((((S)-4-ethyl-8-fluoro-4-hydroxy-9-methyl-3,14-dioxo-3,4,12,14-tetrahydro-1H-pyrano[3′,4′:6,7]-indolizino[1,2-b]quinolin-11-yl)methyl)amino)-2-oxoethoxy)methyl)amino)-1-oxopropan-2-yl)carbamate (A-97-3)

(5S,10S)-10-Cyclopropyl-1-(9H-fluoren-9-yl)-5-methyl-3,6-dioxo-2,9-dioxa-4,7-diazaundecan-11-oleic acid (495 mg, 1.14 mmol) was dissolved in DMF (8 mL), added with (S)-11-(aminomethyl)-9-chloro-4-ethyl-8-fluoro-4-hydroxy-1,12-dihydro-14H-pyrano[3′,4′:6,7]-indolizino[1,2-b]quinolin-3,14(4H)-dione (455.33 mg, 1.15 mmol), DIPEA (875.41 mg, 6.77 mmol) and HATU (858.43 mg, 2.26 mmol), and stirred at room temperature for 4 hours. After the reaction was completed, the reaction solution was directly added with water (100 mL) and EA (50 mL*4) for extraction, washed with water, washed with saturated brine, and concentrated under reduced pressure to obtain the crude product of the title compound (881.0 mg, 583.89 μmol). It was directly used in the next step without purification.

### Step 4: Preparation of (S)-2-amino-N—(((R)-1-cyclopropyl-2-((((S)-4-ethyl-8-fluoro-4-hydroxy-9-methyl-3,14-dioxo-3,4,12,14-tetrahydro-1H-pyrano[3′,4′:6,7]indolizino[1,2-b]-quinolin-11-yl)methyl)amino)-2-oxoethoxy)methyl)propionamide (A-97-4)

(9H-Fluoren-9-yl)methyl ((S)-1-((((R)-1-cyclopropyl-2-(((((S)-4-ethyl-8-fluoro-4-hydroxy-9-methyl-3,14-dioxo-3,4,12,14-tetrahydro-1H-pyrano[3′,4′:6,7]indolizino[1,2-b]-quinolin-11-yl)methyl)amino)-2-oxoethoxy)methyl)amino)-1-oxopropan-2-yl)carbamate (881.0 mg, 583.89 μmol) was dissolved in DMF (4 mL), added with diethylamine (42.7 mg, 583.89 μmol), and stirred at room temperature for 2 hours. After the reaction was completed, the reaction solution was directly purified by preparative high performance liquid chromatography and freeze-dried to obtain the title compound (165.0 mg, 257.97 μmol).

Its structural characterization data are as follows:

MS m/z (ESI): 608.6 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 32 |
| 2.00 | 10 | 90 | 32 |
| 20.00 | 90 | 10 | 32 |

### Step 5: Preparation of N-((4R,9S,12S,15S)-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]-quinolin-11-yl)-9,12-dimethyl-3,8,11,14-tetraoxo-5-oxa-2,7,10,13-tetraazahexadecan-15-yl)-6-(2-(methylsulfonyl)pyrimidin-5-yl)hex-5-ynamide (A-97)

(6-(2-(Methylsulfonyl)pyrimidin-5-yl)hex-5-ynoyl)-L-alanyl-L-alanine (34.54 mg, 84.14 μmol) was dissolved in DMF (2 mL), added with (S)-2-amino-N—(((R)-1-cyclopropyl-2-((((S)-4-ethyl-8-fluoro-4-hydroxy-9-methyl-3,14-dioxo-3,4,12,14-tetrahydro-1H-pyrano[3′,4′:6,7]-indolizino[1,2-b]quinolin-11-yl)methyl)amino)-2-oxoethoxy)methyl)propanamide (50 mg, 76.49 μmol), DIPEA (39.54 mg, 305.97 μmol), and HATU (58.17 mg, 152.99 μmol), and stirred at room temperature for 4 hours. After the reaction was completed, the reaction solution was directly purified by preparative high performance liquid chromatography and freeze-dried to obtain the title compound (20.2 mg, 19.59 μmol).

Its structural characterization data are as follows:

MS m/z (ESI): 1001.06 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 32 |
| 4.00 | 10 | 90 | 32 |
| 24.00 | 90 | 10 | 32 |

### Example 34: Preparation of N-((4R,9S,12S)-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-12-isopropyl-9-methyl-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-98)

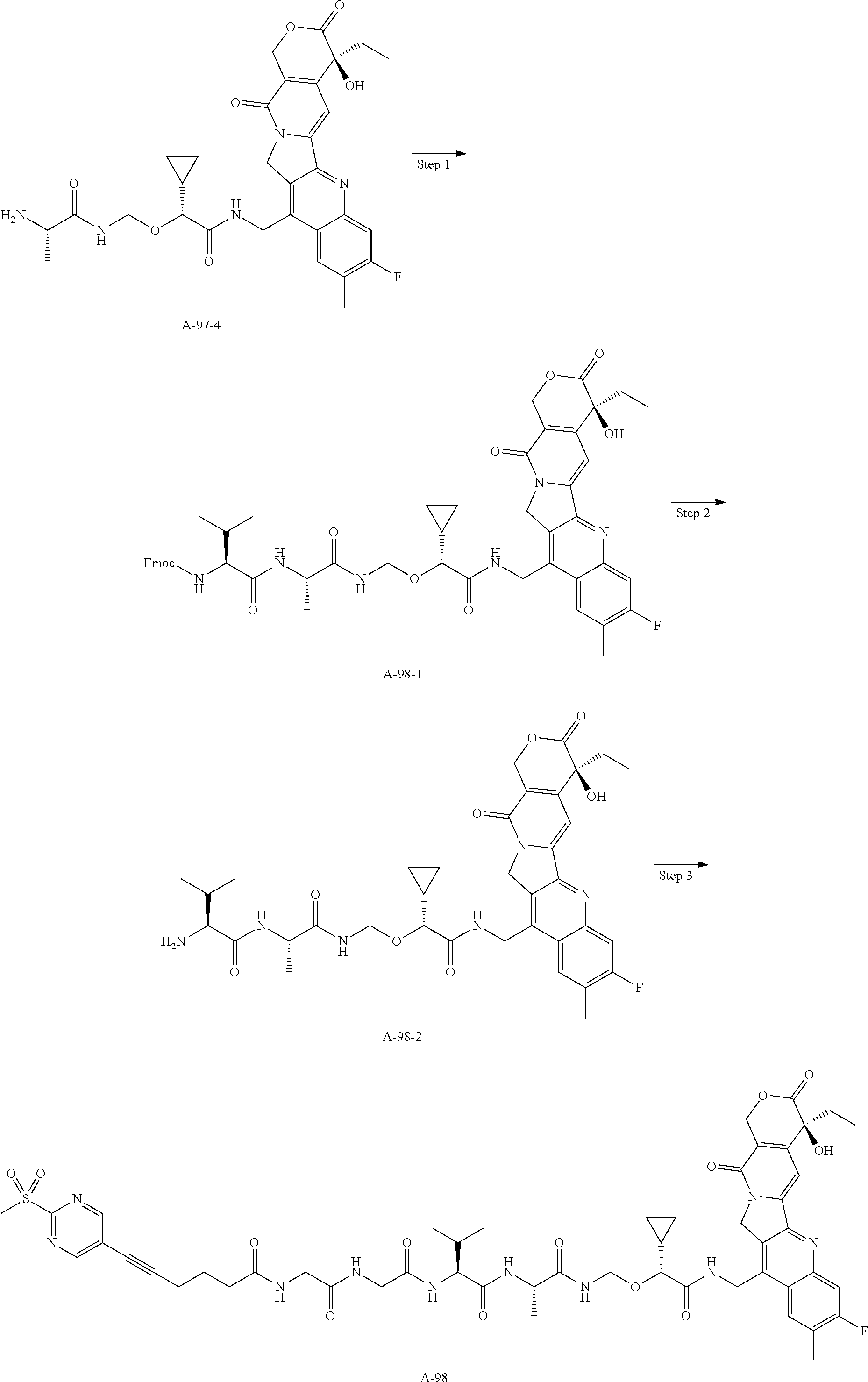

### Step 1: Preparation of (9H-fluoren-9-yl)methyl ((4R,9S,12S)-4-cyclopropyl-1-((S)-4-ethyl-8-fluoro-4-hydroxy-9-methyl-3,14-dioxo-3,4,12,14-tetrahydro-H-pyrano[13′,4′:6,7]-indolizino [1,2-b]quinolin-11-yl)-9,13-dimethyl-3,8,11-trioxo-5-oxa-2,7,10-triazatetradecan-12-yl)carbamate (A-98-1)

To DMF (3 mL), (S)-2-amino-N—(((R)-1-cyclopropyl-2-((((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)amino)-2-oxoethoxy)methyl)propionamide (110 mg, 168.28 μmol), 2,5-dioxopyrrolidin-1-yl (((9H-fluoren-9-yl)methoxy)carbonyl)-L-valinate (183.62 mg, 336.57 μmol) and DIPEA (87.00 mg, 0.673 mmol) were added. After the addition was completed, the reaction was carried out under stirring at 22° C. for 3 hours. After the reaction, the solvent was dried under reduced pressure to obtain the title compound as crude product (150 mg), which was used directly in the next step without purification.

Its structural characterization data are as follows:

MS m/z (ESI): 930.5 [M+H]⁺

### Step 2: Preparation of (S)-2-amino-N—((S)-1-((((R)-1-cyclopropyl-2-((((S)-4-ethyl-8-fluoro-4-hydroxy-9-methyl-3,14-dioxo-3,4,12,14-tetrahydro-1H-pyrano[3′,4′:6,7]indolizino-[1,2-b]quinolin-11-yl)methyl)amino)-2-oxoethoxy)methyl)amino)-1-oxopropan-2-yl)-3-methylbutyramide (A-98-2)

To DMF (3 mL), the crude product of (9H-fluoren-9-yl)methyl ((4R,9S,12S)-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-9,13-dimethyl-3,8,11-trioxo-5-oxa-2,7,10-triazatetradecan-12-yl)carbamate (150 mg) and diethylamine (47 mg, 0.644 mmol) were added in sequence. After the addition was completed, the reaction was carried out under stirring at 22° C. for 2 hours. The reaction solution was purified by flash column chromatography (C18, water/acetonitrile=2/1) to obtain the title compound (100 mg, 0.122 mmol).

Its structural characterization data are as follows:

MS m/z (ESI): 707.3 [M+H]⁺

### Step 3: Preparation of N-((4R,9S,12S)-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-12-isopropyl-9-methyl-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-98)

To DMF (3 mL), (S)-2-amino-N—((S)-1-((((R)-1-cyclopropyl-2-(((((S)-4-ethyl-8-fluoro-4-hydroxy-9-methyl-3,14-dioxo-3,4,12,14-tetrahydro-1H-pyrano[3′,4′:6,7]indolizino[1,2-b]-quinolin-11-yl)methyl)amino)-2-oxoethoxy)methyl)amino)-1-oxopropan-2-yl)-3-methyl-butanamide (80 mg, 0.106 mmol), (6-(2-(methylsulfonyl)pyrimidin-5-yl)hex-5-ynoyl)glycylglycine (44.70 mg, 0.117 mmol), DIPEA (68.67 mg, 0.531 mmol) and HATU (80.81 mg, 0.212 mmol) were added in sequence. After the addition was completed, the reaction was carried out under stirring at 22° C. for 3 hours. The reaction solution was directly purified by preparative high performance liquid chromatography and freeze-dried to obtain the title compound (84 mg, 75.28 μmol).

Its structural characterization data are as follows:

MS m/z (ESI): 1072.5[M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 30 | 70 | 32 |
| 4.00 | 30 | 70 | 32 |
| 24.00 | 90 | 10 | 32 |

### Example 35: Preparation of N-((4R,12S)-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]-quinolin-11-yl)-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-99)

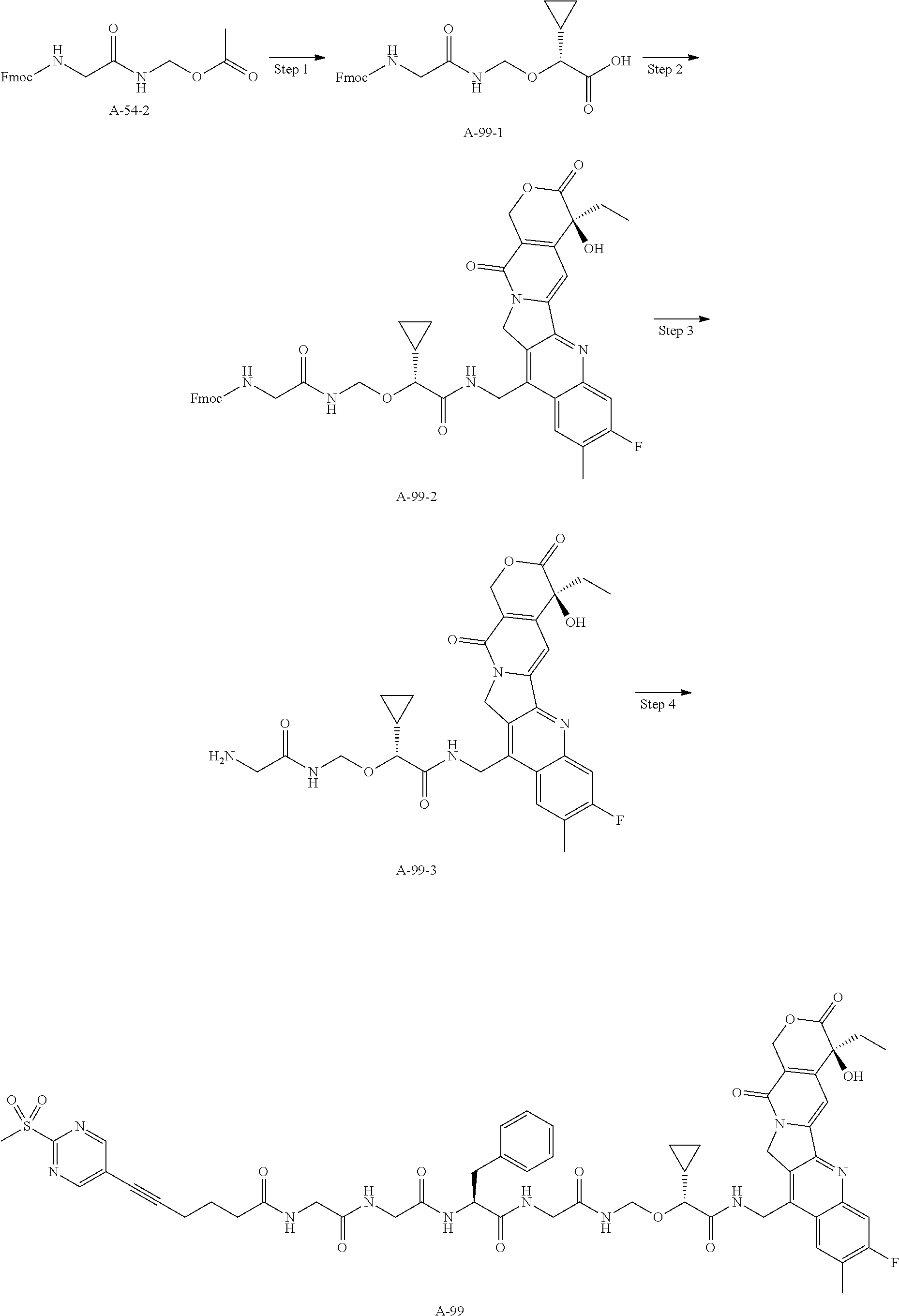

### Step 1: Preparation of (R)-10-cyclopropyl-1-(9H-fluoren-9-yl)-3,6-dioxo-2,9-dioxa-4,7-diazaundecan-11-oic acid (A-99-1)

Under nitrogen protection, (2-((((9H-Fluoren-9-yl)methoxy)carbonyl)amino)acetamido)acetate (500.00 mg, 1.36 mmol), (R)-2-cyclopropyl-2-hydroxyacetic acid (472.80 mg, 4.07 mmol) and p-toluenesulfonic acid monohydrate (129.09 mg, 0.68 mmol) were dissolved in acetonitrile (50 mL), and stirred at room temperature for 0.5 hours. After the reaction was completed, the reaction solution was added with 20 mL of water, extracted with ethyl acetate three times (20 mL×3), concentrated and purified by flash column chromatography (C18, water/acetonitrile=2/1). The components were collected, adjusted to pH 4 with formic acid, extracted with ethyl acetate three times (10 mL×3), washed with 10 mL of brine, dried over anhydrous sodium sulfate, and concentrated under reduced pressure to obtain the title compound (220 mg, 0.52 mmol).

Its structural characterization data are as follows:

MS m/z (ESI): 425.2 [M+H]⁺

### Step 2: Preparation of (9H-fluoren-9-yl)methyl (2-((((R)-1-cyclopropyl-2-((((S)-4-ethyl-8-fluoro-4-hydroxy-9-methyl-3,14-dioxo-3,4,12,14-tetrahydro-1H-pyrano[3′,4′:6,7]indolizino-[1,2-b]quinolin-11-yl)methyl)amino)-2-oxoethoxy)methyl)amino)-2-oxoethyl)carbamate (A-99-2)

(S)-11-(Aminomethyl)-4-ethyl-8-fluoro-4-hydroxy-9-methyl-1,12-dihydro-14H-pyrano-[3′,4′:6,7]indolizino[1,2-b]quinolin-3,14(4H)-dione (233.43 mg, 0.57 mmol) was dissolved in DMF (10 mL), added with DIPEA (200.96 mg, 1.55 mol), stirred for 5 minutes, added with (R)-10-cyclopropyl-1-(9H-fluoren-9-yl)-3,6-dioxo-2,9-dioxa-4,7-diazaundecan-11-oic acid (220 mg, 0.52 mmol) and HATU (393.93 mg, 1.04 mmol), and stirred at room temperature for 2 hours. After the reaction was completed, the title compound (260.00 mg, 0.32 mmol) was obtained by flash column chromatography (C18, water/acetonitrile=0.5).

Its structural characterization data are as follows:

MS m/z (ESI): 816.3 [M+H]⁺

### Step 3: Preparation of (R)-2-((2-aminoacetylamino)methoxy)-2-cyclopropyl-N—(((S)-4-ethyl-8-fluoro-4-hydroxy-9-methyl-3,14-dioxo-3,4,12,14-tetrahydro-1H-pyrano[3′,4′:6,7]-indolizino[1,2-b]quinolin-11-yl)methyl)acetamide (A-99-3)

(9H-Fluoren-9-yl)methyl (2-((((R)-1-cyclopropyl-2-(((((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-methyl)amino)-2-oxoethoxy)methyl)amino)-2-oxoethyl)carbamate (100.00 mg, 0.12 mmol) was dissolved in DMF (3 mL), added with diethylamine (44.80 mg, 0.61 mmol), and stirred at room temperature for 1 hour. After the reaction was completed, the title compound (35.00 mg, 0.05 mmol) was obtained after purification by flash column chromatography (C18, water/acetonitrile=0.7) and freeze-drying.

Its structural characterization data are as follows:

MS m/z (ESI): 594.3 [M+H]⁺

### Step 4: Preparation of N-((4R,12S)-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]-quinolin-11-yl)-3,8,11,14,17-pentaoxo-5-oxa-2,7,10,13,16-pentaazaoctadecan-18-yl)-6-(2-(methylsulfonyl)pyrimidin-5-yl)hex-5-ynamide (A-99)

(R)-2-((2-Aminoacetylamino)methoxy)-2-cyclopropyl-N—(((S)-4-ethyl-8-fluoro-4-hydroxy-9-methyl-3,14-dioxo-3,4,12,14-tetrahydro-1H-pyrano[3′,4′:6,7]indolizino[1,2-b]-quinolin-11-yl)methyl)acetamide (40.00 mg, 0.06 mmol) was dissolved in DMF (3 mL), added with DIPEA (24.25 mg, 0.19 mol), stirred for 5 minutes, then added with (6-(2-(methylsulfonyl) pyrimidin-5-yl)hex-5-ynoyl)glycylglycyl-L-phenylalanine (36.43 mg, 0.07 mmol) and HATU (47.53 mg, 0.13 mmol), and stirred at room temperature for 2 hours. After the reaction was completed, the reaction solution was directly purified by preparative high performance liquid chromatography and freeze-dried to obtain the title compound (22.90 mg, 0.02 mmol).

Its structural characterization data are as follows:

MS m/z (ESI): 1106.4 [M+H]⁺

Its preparation method was as follows:

Chromatographic column: Waters SunFire Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 30 | 70 | 32 |
| 4.00 | 30 | 70 | 32 |
| 24.00 | 90 | 10 | 32 |

### Example 36: Preparation of N-((5S,13S)-13-benzyl-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-5-methyl-3,9,12,15,18-pentaoxo-6-oxa-2,8,11,14,17-pentaazanonadecan-19-yl)-6-(2-(methylsulfonyl)pyrimidin-5-yl)hex-5-ynamide (A-125)

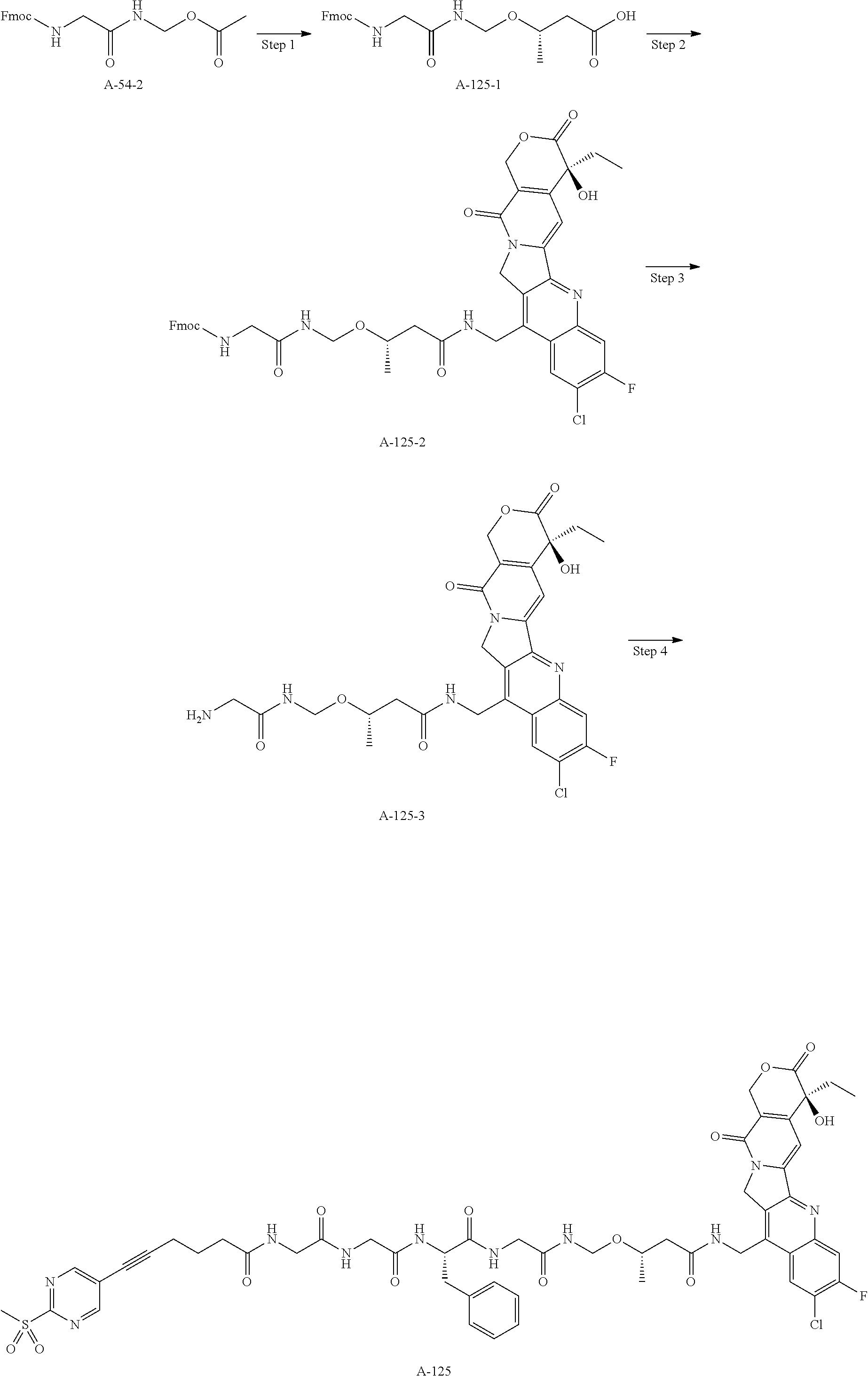

### Step 1: Preparation of (S)-1-(9H-fluoren-9-yl)-10-methyl-3,6-dioxo-2,9-dioxa-4,7-diazadodecan-12-oic acid (A-125-1)

Methyl (2-((((9H-fluoren-9-yl)methoxy)carbonyl)amino)acetamido)acetate (500 mg, 1.36 mmol) and (S)-3-hydroxybutyric acid (282.6 mg, 2.71 mmol) were dissolved in tetrahydrofuran (10 mL), added with p-toluenesulfonic acid monohydrate (129.1 mg, 678.64 μmol), heated to 50° C. and reacted for 6 hours. After most of the tetrahydrofuran was removed by concentration, and the residue was dissolved in an appropriate amount of MeOH/H₂O, purified by C18 reverse column (ACN/H₂O=5 to 80%, 0.1% formic acid aqueous solution), and freeze-dried to obtain the title compound (270 mg, 0.65 mmol).

Its structural characterization data are as follows:

MS m/z (ESI): 435.1 [M+Na]⁺

### Step 2: Preparation of (9H-fluoren-9-yl)methyl (2-(((((S)-4-(((((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)amino)-4-oxobutan-2-yl)oxy)methyl)amino)-2-oxoethyl)carbamate (A-125-2)

(S)-11-(Aminomethyl)-9-chloro-4-ethyl-8-fluoro-4-hydroxy-1,12-dihydro-14H-pyrano-[3′,4′:6,7]indolizino[1,2-b]quinolin-3,14(4H)-dione (100 mg, 0.18 mmol) and (S)-1-(9H-fluoren-9-yl)-10-methyl-3,6-dioxo-2,9-dioxa-4,7-diazadodecan-12-oic acid (91.00 mg, 0.22 mmol) were dissolved in DMF (3 mL), added with HATU (139.8 mg, 0.37 mmol) and DIPEA (71.3 mg, 0.55 mmol), and reacted at 25° C. for 1 hour. The reaction solution was directly purified by C18 reverse column (ACN/H₂O=5 to 80%, 0.05% formic acid aqueous solution) and freeze-dried to obtain the title compound (98 mg, 0.12 mmol).

Its structural characterization data are as follows:

MS m/z (ESI): 824.3[M+H]⁺

### Step 3: Preparation of (S)-3-((2-aminoacetylamino)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)butyramide (A-125-3)

(9H-Fluoren-9-yl)methyl (2-(((((S)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-amino)-4-oxobutan-2-yl)oxy)methyl)amino)-2-oxoethyl)carbamate (98 mg, 0.12 mmol) was dissolved in DMF (2 mL), added with diethylamine (43.5 mg, 594.48 μmol), and reacted at 25° C. for 2 hours. The reaction solution was directly purified by C18 reverse column (ACN/H₂O=10 to 80%, 0.1% ammonium bicarbonate aqueous solution) and freeze-dried to obtain the title compound (51 mg, 0.085 mmol).

Its structural characterization data are as follows:

MS m/z (ESI): 602.2[M+H]⁺

### Step 4: Preparation of N-((5S,13S)-13-benzyl-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-5-methyl-3,9,12,15,18-pentaoxo-6-oxa-2,8,11,14,17-pentaazanonadecan-19-yl)-6-(2-(methyl-sulfonyl)pyrimidin-5-yl)hex-5-ynamide (A-125)

(6-(2-(Methylsulfonyl)pyrimidin-5-yl)hex-5-ynoyl)glycylglycyl-L-phenylalanine (49.4 mg, 0.093 mmol) was dissolved in DMF (2 mL), added with HATU (64.4 mg, 0.17 mmol) and DIPEA (21.90 mg, 0.17 mmol), stirred for 5 minutes, then added with (S)-3-((2-aminoacetylamino)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)butanamide (51 mg, 0.085 mmol), and reacted at 25° C. for 1 hour. The reaction solution was directly purified by preparative high performance liquid chromatography and freeze-dried to obtain the title compound (39.2 mg, 0.034 mmol).

Its structural characterization data are as follows:

MS m/z (ESI): 1113.4 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 25 | 75 | 30 |
| 2.00 | 25 | 75 | 30 |
| 18.00 | 85 | 15 | 30 |

### Example 37: Preparation of N-((5R,13S)-13-benzyl-1-((S)-9-chloro-4-ethyl-8-fluoro-4-hydroxy-3,14-dioxo-3,4,12,14-tetrahydro-TH-pyrano[3′,4′:6,7]indolizino[1,2-b]quinolin-11-yl)-5-methyl-3,9,12,15,18-pentaoxo-6-oxa-2,8,11,14,17-pentaazanonadecan-19-yl)-6-(2-(methylsulfonyl)pyrimidin-5-yl)hex-5-ynamide (A-126)

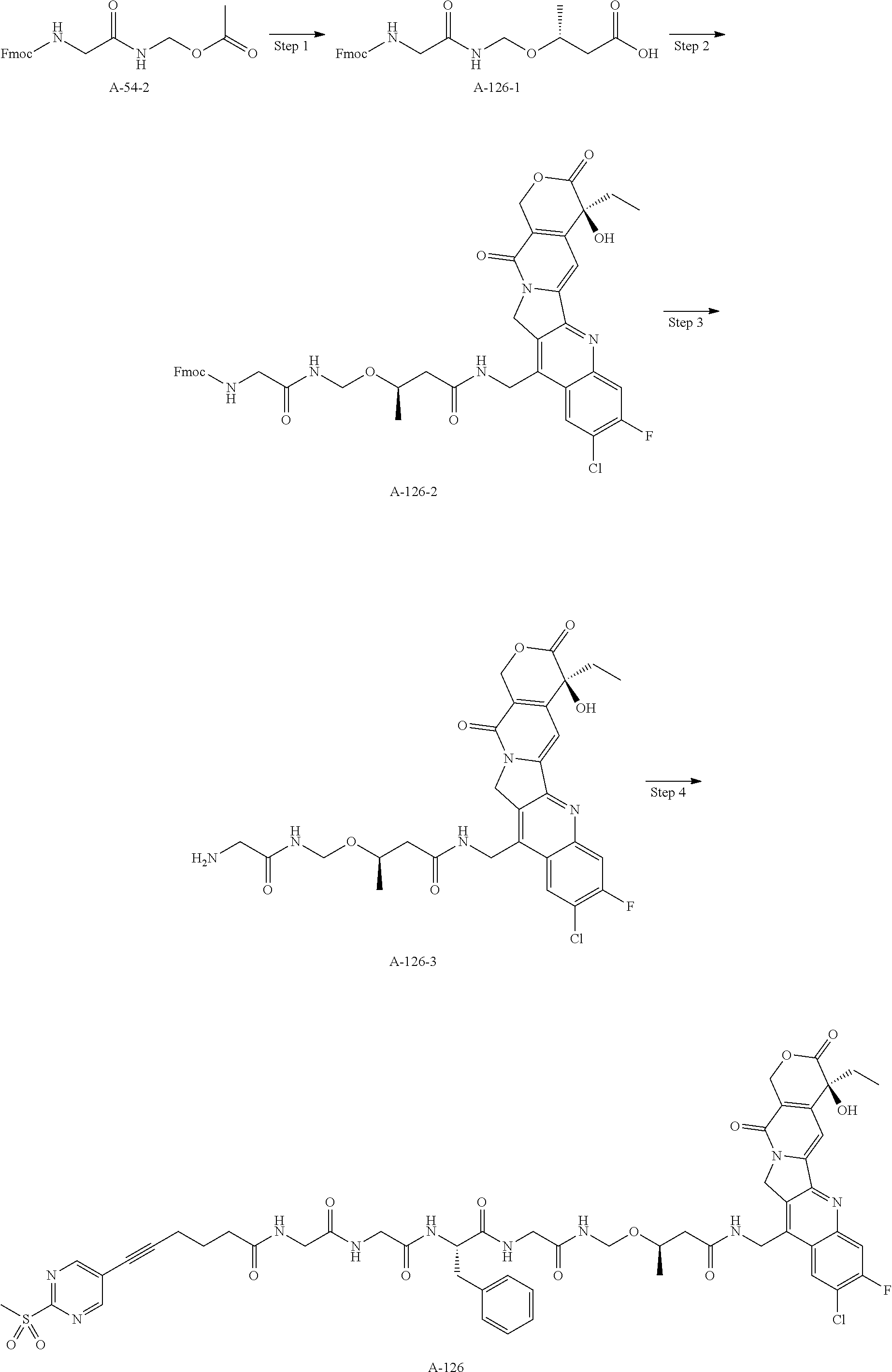

### Step 1: Preparation of (R)-1-(9H-fluoren-9-yl)-10-methyl-3,6-dioxo-2,9-dioxa-4,7-diazadodecan-12-oic acid (A-126-1)

(R)-3-hydroxybutyric acid (339.1 mg, 3.26 mmol) and methyl (2-((((9H-fluoren-9-yl) methoxy)carbonyl)amino)acetamido)acetate (400 mg, 1.09 mmol) were dissolved in THF (20 mL), then added with p-toluenesulfonic acid monohydrate (62.0 mg, 0.32 mmol), and reacted at 25° C. for 3 hours. The reaction product was added with water (20 ml) and ethyl acetate (30 ml) for extraction, and concentrated to obtain a crude product, which was purified by preparative high performance liquid chromatography and freeze-dry to obtain the title compound (295 mg, 0.72 mmol).

Its structural characterization data are as follows:

MS m/z (ESI): 430.0 [M+H₂O]⁺

Its preparation method was as follows:

Chromatographic column: Waters Xbridge Prep C18
OBD (5 μm*19 mm*150 mm)
Mobile phase A: acetonitrile; Mobile phase B: water
(0.05% ammonium bicarbonate)

| Time | Mobile phase | Mobile phase | Flow rate |
|---|---|---|---|
| [min] | A [%] | B [%] | [mL/min] |
| 0.00 | 20 | 80 | 30 |
| 2.00 | 20 | 80 | 30 |
| 18.00 | 80 | 20 | 30 |

### Step 2: Preparation of (9H-fluoren-9-yl)methyl (2-(((((R)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)amino)-4-oxobutan-2-yl)oxy)methyl)amino)-2-oxoethyl)carbamate (A-126-2)

(S)-11-(Aminomethyl)-9-chloro-4-ethyl-8-fluoro-4-hydroxy-1,12-dihydro-14H-pyrano[3′,4′:6,7]indolizino[1,2-b]quinolin-3,14(4H)-dione (100 mg, 0.18 mmol) and (R)-1-(9H-fluoren-9-yl)-10-methyl-3,6-dioxo-2,9-dioxa-4,7-diazadodecan-12-oic acid (91.00 mg, 0.22 mmol) were dissolved in DMF (3 mL), added with HATU (139.8 mg, 0.37 mmol) and DIPEA (71.3 mg, 0.55 mmol), and reacted at 25° C. for 1 hour. The reaction solution was directly purified by C18 reverse column (ACN/H₂O=5 to 80%, 0.05% formic acid aqueous solution) and freeze-dried to obtain the title compound (72 mg, 0.087 mmol).

Its structural characterization data are as follows:

MS m/z (ESI): 824.3[M+H]⁺

### Step 3: Preparation of (R)-3-((2-aminoacetylamino)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)butanamide (A-126-3)

(9H-Fluoren-9-yl)methyl (2-(((((R)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-amino)-4-oxobutane-2-yl)oxy)methyl)amino)-2-oxoethyl)carbamate (72 mg, 0.088 mmol) was dissolved in DMF (2 mL), added with diethylamine (31.9 mg, 0.44 mmol), and reacted at 25° C. for 2 hours. The reaction solution was directly purified by C18 reverse column (ACN/H₂O=10 to 80%, 0.1% ammonium bicarbonate aqueous solution) and freeze-dried to obtain the title compound (44 mg, 0.073 mmol).

Its structural characterization data are as follows:

MS m/z (ESI): 602.2[M+H]⁺

### Step 4: Preparation of N-((5R,13S)-13-benzyl-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-5-methyl-3,9,12,15,18-pentaoxo-6-oxa-2,8,11,14,17-pentaazanonadecan-19-yl)-6-(2-(methylsulfonyl)pyrimidin-5-yl)hex-5-ynamide (A-126)

(6-(2-(Methylsulfonyl)pyrimidin-5-yl)hex-5-ynoyl)glycylglycyl-L-phenylalanine (39.7 mg, 0.075 mmol) was dissolved in DMF (2 mL), added with HATU (51.8 mg, 0.14 mmol) and DIPEA (17.6 mg, 0.14 mmol), stirred for 5 minutes, then added (R)-3-((2-aminoacetylamino) methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)butanamide (41 mg, 0.068 mmol), and reacted at 25° C. for 1 hour. The reaction solution was directly purified by preparative high performance liquid chromatography and freeze-dried to obtain the title compound (31.3 mg, 0.027 mmol).

Its structural characterization data are as follows:

MS m/z (ESI): 1113.4 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 30 | 70 | 32 |
| 4.00 | 30 | 70 | 32 |
| 24.00 | 90 | 10 | 32 |

### Example 38: Preparation of N-((5S,10S,13S)-1-((S)-9-chloro-4-ethyl-8-fluoro-4-hydroxy-3,14-dioxo-3,4,12,14-tetrahydro-TH-pyrano[3′,4′:6,7]indolizino[1,2-b]quinolin-11-yl)-13-isopropyl-5,10-dimethyl-3,9,12,15,18-pentaoxo-6-oxa-2,8,11,14,17-pentaazanonadecan-19-yl)-6-(2-(methylsulfonyl)pyrimidin-5-yl)hex-5-ynamide (A-135)

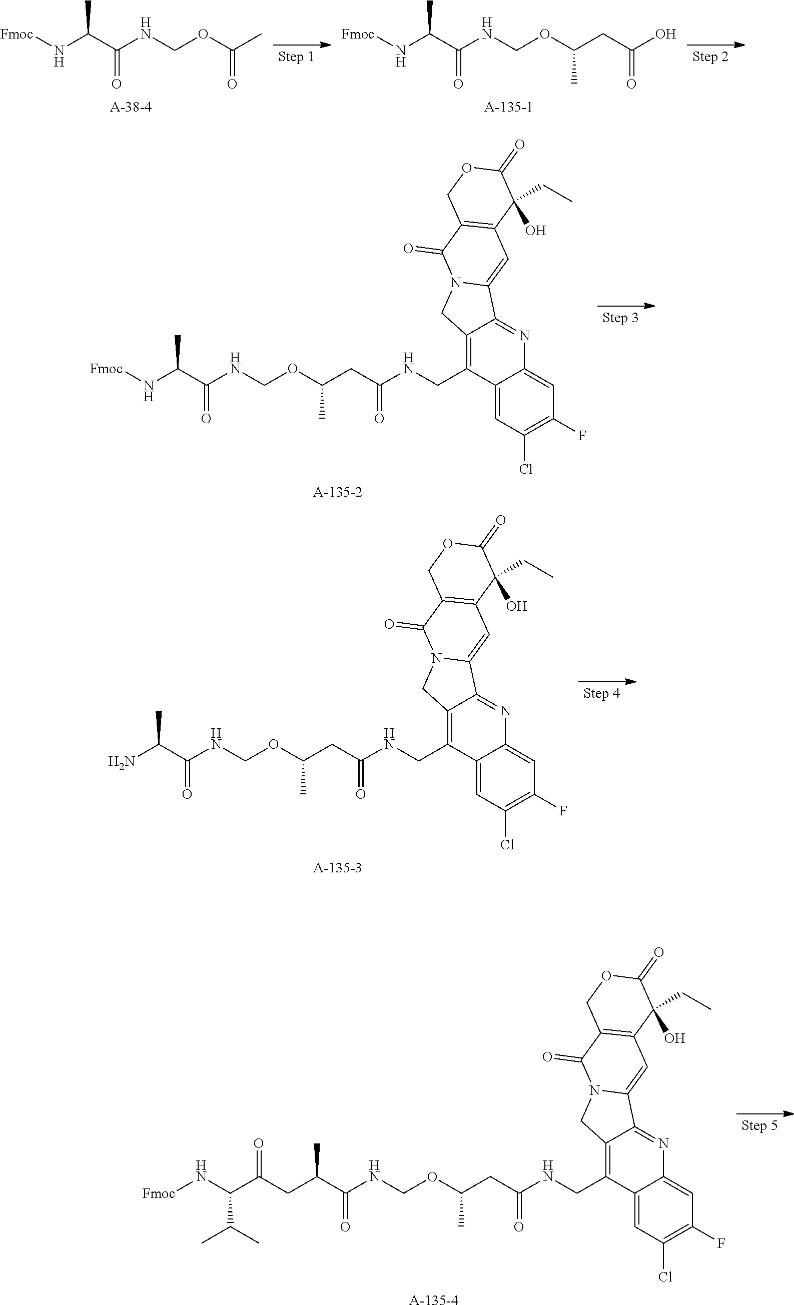

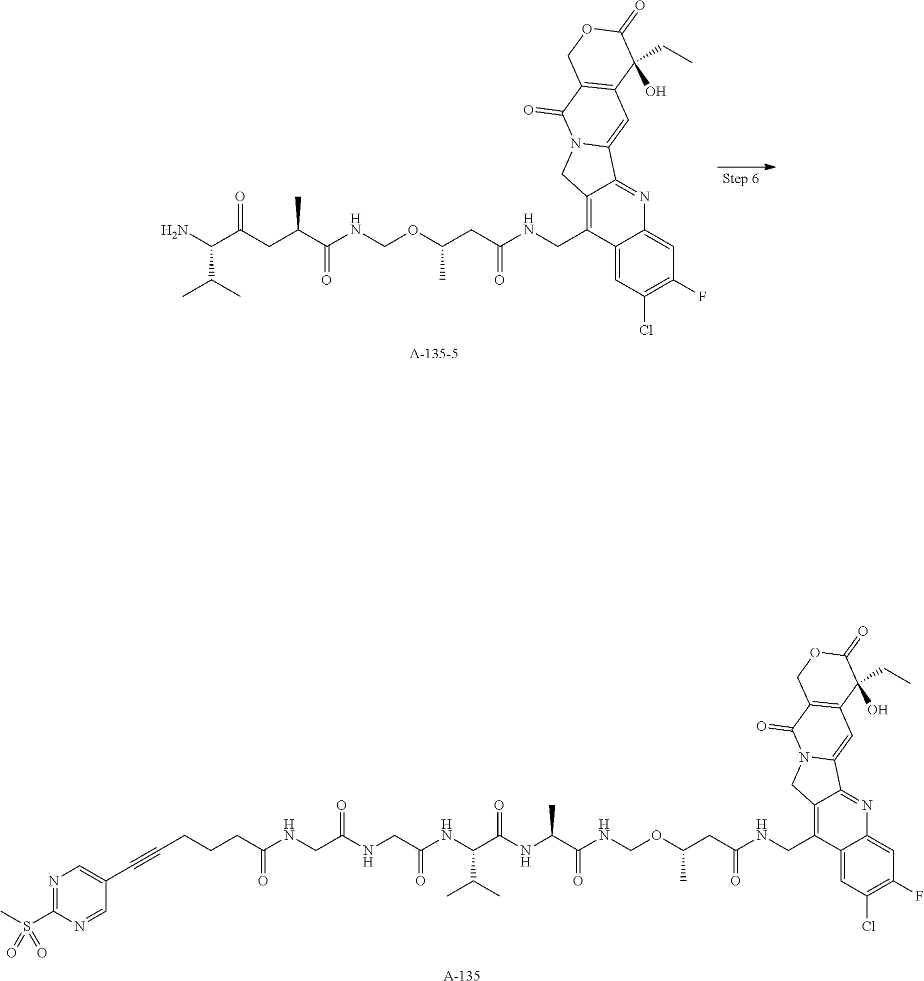 [SMILES: CC[C@@]1(O)C(=O)OCc2c1cc1n(c2=O)Cc2c-1nc1cc(F)c(Cl)cc1c2CNC(=O)C[C@H](C)OCNC(=O)[C@H](C)CC(=O)[C@@H](N)C(C)C ; "A-135-5" ; CC[C@@]1(O)C(=O)OCc2c1cc1n(c2=O)Cc2c-1nc1cc(F)c(Cl)cc1c2CNC(=O)C[C@H](C)OCNC(=O)[C@H](C)NC(=O)[C@@H](NC(=O)CNC(=O)CNC(=O)CCCC#Cc1cnc(S(C)(=O)=O)nc1)C(C)C ; "A-135"]

### Step 1: Preparation of (5S,10)-1-(9H-fluoren-9-yl)-5,10-dimethyl-3,6-dioxo-2,9-dioxa-4,7-diazadodecan-12-oic acid (A-135-1)

Methyl (S)-(2-((((9H-fluoren-9-yl)methoxy)carbonyl)amino)propionamido)acetate (500 mg, 1.31 mmol) and (S)-3-hydroxybutyric acid (204.2 mg, 1.96 mmol) were dissolved in THF (10 mL), added with p-toluenesulfonic acid monohydrate (45.0 mg, 0.26 mmol), heated to 50° C. and reacted for 6 hours. The crude product was concentrated and purified by C18 reverse column (ACN/H₂O=5 to 80%, 0.1% formic acid aqueous solution) and freeze-dried to obtain the title compound (188 mg, 0.44 mmol).

Its structural characterization data are as follows:

MS m/z (ESI): 449.2[M+Na]⁺

### Step 2: Preparation of (9H-fluoren-9-yl)methyl ((S)-1-(((((S)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)amino)-4-oxobutan-2-yl)oxy)methyl)amino)-1-oxopropan-2-yl)-carbamate (A-135-2)

(S)-11-(Aminomethyl)-9-chloro-4-ethyl-8-fluoro-4-hydroxy-1,12-dihydro-14H-pyrano-[3′,4′:6,7]indolizino[1,2-b]quinolin-3,14(4H)-dione (100 mg, 0.18 mmol) and A-135-1 (94.1 mg, 0.22 mmol) were dissolved in DMF (3 mL), added with HATU (139.8 mg, 0.37 mmol) and DIPEA (71.3 mg, 0.55 mmol), and reacted at 25° C. for 1 hour. The reaction solution was directly purified by C18 reverse column (ACN/H₂O=5 to 80%, 0.05% formic acid aqueous solution) and freeze-dried to obtain the title compound (132 mg, 0.16 mmol).

Its structural characterization data are as follows:

MS m/z (ESI): 838.3 [M+H]⁺

### Step 3: Preparation of (S)-3-(((S)-2-aminopropionamido)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)butanamide (A-135-3)

(9H-Fluoren-9-yl)methyl ((S)-1-(((((S)-4-(((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-amino)-4-oxobutan-2-yl)oxy)methyl)amino)-1-oxopropan-2-yl)carbamate (132 mg, 0.16 mmol) was dissolved in DMF (2 mL), added with diethylamine (58 mg, 0.79 mmol), and reacted at 25° C. for 2 hours. The reaction solution was directly purified by C18 reverse column (ACN/H₂O=10 to 80%, 0.1% ammonium bicarbonate aqueous solution) and freeze-dried to obtain the title compound (42 mg, 0.068 mmol).

Its structural characterization data are as follows:

MS m/z (ESI): 616.3 [M+H]⁺

### Step 4: Preparation of (9H-fluoren-9-yl)methyl ((5S,10S,13S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)-5,10,14-trimethyl-3,9,12-trioxo-6-oxa-2,8,11-triazapentadecan-13-yl)carbamate (A-135-4)

(S)-3-(((S)-2-Aminopropionamido)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)butanamide (42.0 mg, 0.068 mmol) and (((9H-fluoren-9-yl)methoxy)carbonyl)-L-valine (27.77 mg, 0.081 mmol) were dissolved in DMF (2 mL), added with HATU (51.8 mg, 0.14 mmol) and DIPEA (26.4 mg, 0.204 mmol), and reacted at 25° C. for 2 hours. The reaction solution was directly purified by C18 reverse column (ACN/H₂O=10 to 80%, 0.1% ammonium bicarbonate aqueous solution) and freeze-dried to obtain the title compound (41 mg, 0.044 mmol).

Its structural characterization data are as follows:

MS m/z (ESI): 937.3[M+H]⁺

### Step 5: Preparation of (S)-2-amino-N—((S)-1-(((((S)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)amino)-4-oxobutan-2-yl)oxy)methyl)amino)-1-oxopropan-2-yl)-3-methylbutanamide (A-135-5)

(9H-Fluoren-9-yl)methyl ((5S,10S,13S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-5,10,14-trimethyl-3,9,12-trioxo-6-oxa-2,8,11-triazapentadecan-13-yl)carbamate (41 mg, 0.044 mmol) was dissolved in DMF (1 mL), added with diethylamine (16.0 mg, 0.22 mmol), reacted at 25° C. for 2 hours. The reaction solution was directly purified by C18 reverse column (ACN/H₂O=10 to 80%, 0.1% ammonium bicarbonate aqueous solution) and freeze-dried to obtain the title compound (16 mg, 0.022 mmol).

Its structural characterization data are as follows:

MS m/z (ESI): 715.2[M+H]⁺

### Step 6: Preparation of N-((5S,10S,13S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-13-isopropyl-5,10-dimethyl-3,9,12,15,18-pentaoxo-6-oxa-2,8,11,14,17-pentaazanonadecan-19-yl)-6-(2-(methylsulfonyl)pyrimidin-5-yl)hex-5-ynamide (A-135)

(6-(2-(Methylsulfonyl)pyrimidin-5-yl)hex-5-ynoyl)glycylglycine (9.41 mg, 0.024 mmol) was dissolved in DMF (1 mL), added with HATU (17.0 mg, 0.045 mmol) and DIPEA (5.8 mg, 0.045 mmol), stirred for 5 minutes, then added with (S)-2-amino-N—((S)-1-(((((S)-4-((((S)-9-chloro-4-ethyl-8-fluoro-4-hydroxy-3,14-dioxo-3,4,12,14-tetrahydro-1H-pyrano[3′,4′:6,7]-indolizino[1,2-b]quinolin-11-yl)methyl)amino)-4-oxobutan-2-yl)oxy)methyl)amino)-1-oxopropan-2-yl)-3-methylbutanamide (16.00 mg, 0.022 mmol), and reacted at 25° C. for 1 hour.

The reaction solution was directly purified by preparative high performance liquid chromatography and freeze-dried to obtain the title compound (8.10 mg, 0.0073 mmol).

Its structural characterization data are as follows:

MS m/z (ESI): 1079.3 [M+H]⁺

Its preparation method was as follows:

Chromatographic column: Waters Xbridge Prep C18
OBD (5 μm * 19 mm * 150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 30 | 70 | 32 |
| 4.00 | 30 | 70 | 32 |
| 24.00 | 90 | 10 | 32 |

### Example 39: Preparation of ((5R,10S,13S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-13-isopropyl-5,10-dimethyl-3,9,12,15,18-pentaoxo-6-oxa-2,8,11,14,17-pentaazanonadecan-19-yl)-6-(2-(methylsulfonyl)pyrimidin-5-yl)hex-5-ynamide A-136)

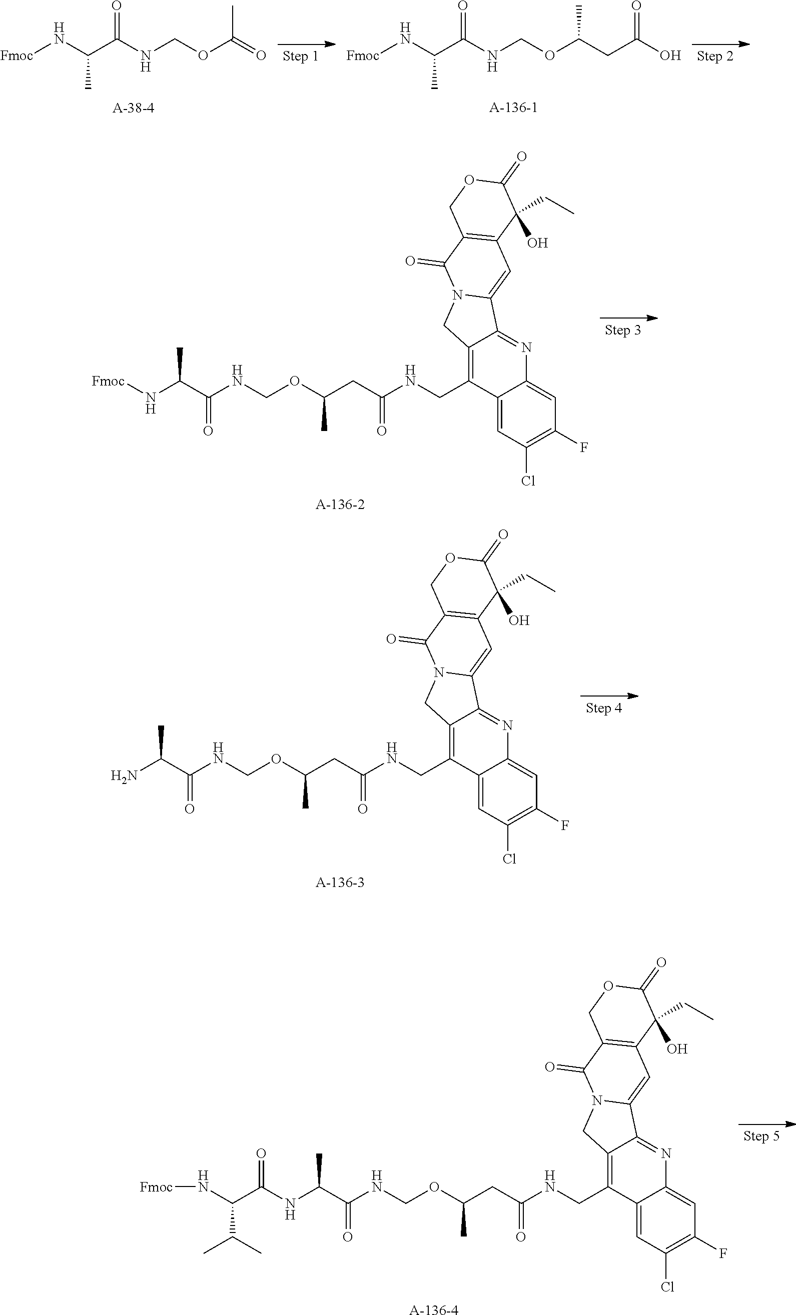

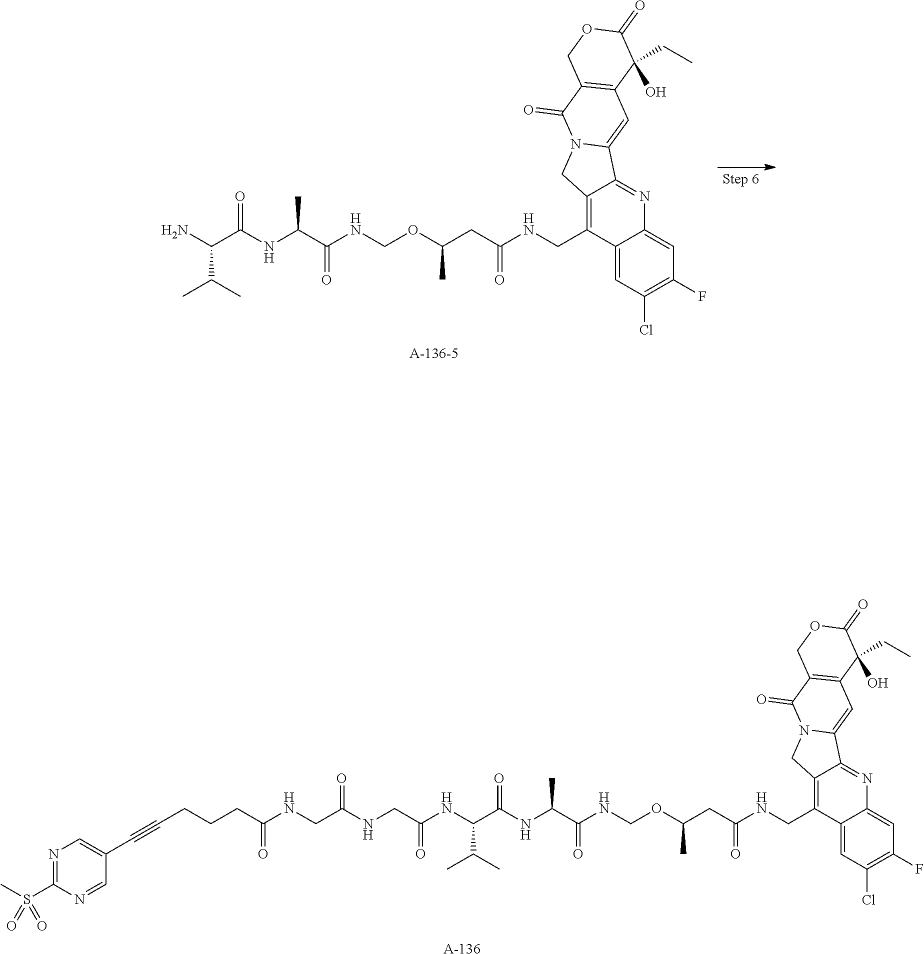 [SMILES: CC[C@@]1(O)C(=O)OCc2c1cc1n(c2=O)Cc2c-1nc1cc(F)c(Cl)cc1c2CNC(=O)C[C@@H](C)OCNC(=O)[C@H](C)NC(=O)[C@@H](N)C(C)C ; "A-136-5" ; CC[C@@]1(O)C(=O)OCc2c1cc1n(c2=O)Cc2c-1nc1cc(F)c(Cl)cc1c2CNC(=O)C[C@@H](C)OCNC(=O)[C@H](C)NC(=O)[C@@H](NC(=O)CNC(=O)CNC(=O)CCCC#Cc1cnc(S(C)(=O)=O)nc1)C(C)C ; "A-136"]

### Step 1: Preparation of (5S,10R)-1-(9H-fluoren-9-yl)-5,10-dimethyl-3,6-dioxo-2,9-dioxa-4,7-diazadodecan-12-oic acid (A-136-1)

(R)-3-Hydroxybutyric acid (571.69 mg, 5.49 mmol) and methyl (S)-(2-((((9H-fluoren-9-yl)methoxy)carbonyl)amino)propionamido)acetate (700 mg, 1.83 mmol) were dissolved in THF (25 mL), then added with p-toluenesulfonic acid monohydrate (52.23 mg, 274.58 μmol), and stirred at room temperature for 2 hours. After the reaction was completed, the reaction solution was added with water and ethyl acetate for extraction, and concentrated to obtain a crude product. The reaction solution was directly purified by preparative high-performance liquid chromatography and freeze-dried to obtain the title compound (248 mg, 581.53 μmol).

Its structural characterization data are as follows:

MS m/z (ESI): 444.3[M+18]⁺

Its preparation method was as follows:

Chromatographic column: Waters Xbridge Prep C18
OBD (5 μm*19 mm*150 mm)
Mobile phase A: acetonitrile; Mobile phase B: water
(0.05% ammonium bicarbonate)

| Time | Mobile phase | Mobile phase | Flow rate |
|---|---|---|---|
| [min] | A [%] | B [%] | [mL/min] |
| 0.00 | 20 | 80 | 30 |
| 4.00 | 20 | 80 | 30 |
| 24.00 | 90 | 10 | 30 |

### Step 2: Preparation of (9H-fluoren-9-yl)methyl ((S)-1-(((((R)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]-quinolin-11-yl)methyl)amino)-4-oxobutan-2-yl)oxy)methyl)amino)-1-oxopropan-2-yl)-carbamate (A-136-2)

(S)-11-(Aminomethyl)-9-chloro-4-ethyl-8-fluoro-4-hydroxy-1,12-dihydro-14H-pyrano-[3′,4′:6,7]indolizino[1,2-b]quinolin-3,14(4H)-dione (300 mg, 597.04 μmol) and DIPEA (299.34 mg, 2.32 mmol) were dissolved in DMF (10 mL), then (5S,10R)-1-(9H-fluoren-9-yl)-5,10-dimethyl-3,6-dioxo-2,9-dioxa-4,7-diazadodecan-12-oic acid (246.94 mg, 579.04 μmol) and HATU (440.35 mg, 1.16 mmol) were added to the reaction system, and stirred at 25° C. for 2 hours. The reaction solution was directly purified by preparative high performance liquid chromatography and freeze-dried to obtain the title compound (244 mg, 291.07 μmol).

Its structural characterization data are as follows:

MS m/z (ESI): 838.4 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 40 | 60 | 32 |
| 4.00 | 40 | 60 | 32 |
| 24.00 | 90 | 10 | 32 |

### Step 3: Preparation of (R)-3-(((S)-2-aminopropionamido)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)butanamide (A-136-3)

(9H-Fluoren-9-yl)methyl ((S)-1-(((((R)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)amino)-4-oxobutan-2-yl)oxy)methyl)amino)-1-oxopropan-2-yl)carbamate (244 mg, 291.07 μmol) was dissolved in DMF (10 mL), added with diethylamine (212.89 mg, 2.91 mmol), and stirred at room temperature for 2 hours. After the reaction was completed, the title compound (110 mg, 166.15 μmol) was obtained by flash column chromatography (C18, acetonitrile/water=0-40%, 0.05% formic acid) and freeze-drying.

Its structural characterization data are as follows:

MS m/z (ESI): 616.3[M+H]⁺

### Step 4: Preparation of (9H-fluoren-9-yl)methyl ((5R,10S,13S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-5,10,14-trimethyl-3,9,12-trioxo-6-oxa-2,8,11-triazapentadecan-13-yl)carbamate (A-136-4)

(R)-3-(((S)-2-Aminopropionamido)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)butanamide (110 mg, 166.15 μmol) was dissolved in DMF (6 mL), added with 2,5-dioxopyrrolidin-1-yl ((9H-fluoren-9-yl)methoxy)carbonyl)-L-valine (255.94 mg, 498.44 μmol) and DIPEA (85.89 mg, 664.59 μmol), and stirred at room temperature for 1 hour. After the reaction was completed, the reaction solution was directly purified by preparative high performance liquid chromatography and freeze-dried to obtain the title compound (95 mg, 101.34 μmol).

Its structural characterization data are as follows:

MS m/z (ESI): 937.3[M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 20 | 80 | 30 |
| 4.00 | 20 | 80 | 30 |
| 24.00 | 90 | 10 | 30 |

### Step 5: Preparation of (S)-2-amino-N—((S)-1-(((((R)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)amino)-4-oxobutan-2-yl)oxy)methyl)amino)-1-oxopropan-2-yl)-3-methylbutanamide (A-136-5)

(9H-Fluoren-9-yl)methyl ((5R,10S,13S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-5,10,14-trimethyl-3,9,12-trioxo-6-oxa-2,8,11-triazapentadecan-13-yl)carbamate (95 mg, 101.34 μmol) was dissolved in DMF (5 mL), added with diethylamine (74.12 mg, 1.01 mmol), and stirred at room temperature for 1.5 hours. After the reaction was completed, the title compound (70 mg, 91.96 μmol) was obtained by flash column chromatography (C18, acetonitrile/water=0-45, 0.05% formic acid) and freeze-drying.

Its structural characterization data are as follows:

MS m/z (ESI): 715.3 [M+H]⁺

### Step 6: Preparation of ((5R,10S,13S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-13-isopropyl-5,10-dimethyl-3,9,12,15,18-pentaoxo-6-oxa-2,8,11,14,17-pentaazanonadecan-19-yl)-6-(2-(methylsulfonyl)pyrimidin-5-yl)hex-5-ynamide (A136)

6(S)-2-Amino-N—((S)-1-(((((R)-4-((((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)amino)-4-oxobutan-2-yl)oxy)methyl)amino)-1-oxopropan-2-yl)-3-methylbutanamide (30 mg, 39.41 μmol) and DIPEA (25.47 mg, 197.06 μmol) were dissolved in DMF (5 mL), stirred for 10 minutes, then (6-(2-(methylsulfonyl)pyrimidin-5-yl)hex-5-ynoyl)glycylglycine (15.07 mg, 39.41 μmol) and HATU (29.97 mg, 78.82 μmol) were added to the reaction system, and the reaction was carried out under stirring at 25° C. for 2 hours. The reaction solution was directly purified by preparative high performance liquid chromatography and freeze-dried to obtain the title compound (9.50 mg, 8.31 μmol).

Its structural characterization data are as follows:

MS m/z (ESI): 1080.4[M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 25 | 80 | 30 |
| 3.00 | 25 | 80 | 30 |
| 18.00 | 80 | 20 | 30 |

### Example 40: Preparation of N-((5S,10S,13S,16S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-5,10,13-trimethyl-3,9,12,15-tetraoxo-6-oxa-2,8,11,14-tetraazaheptadecan-16-yl)-6-(2-methylsulfonyl)pyrimidin-5-yl)hex-5-ynamide (A-145)

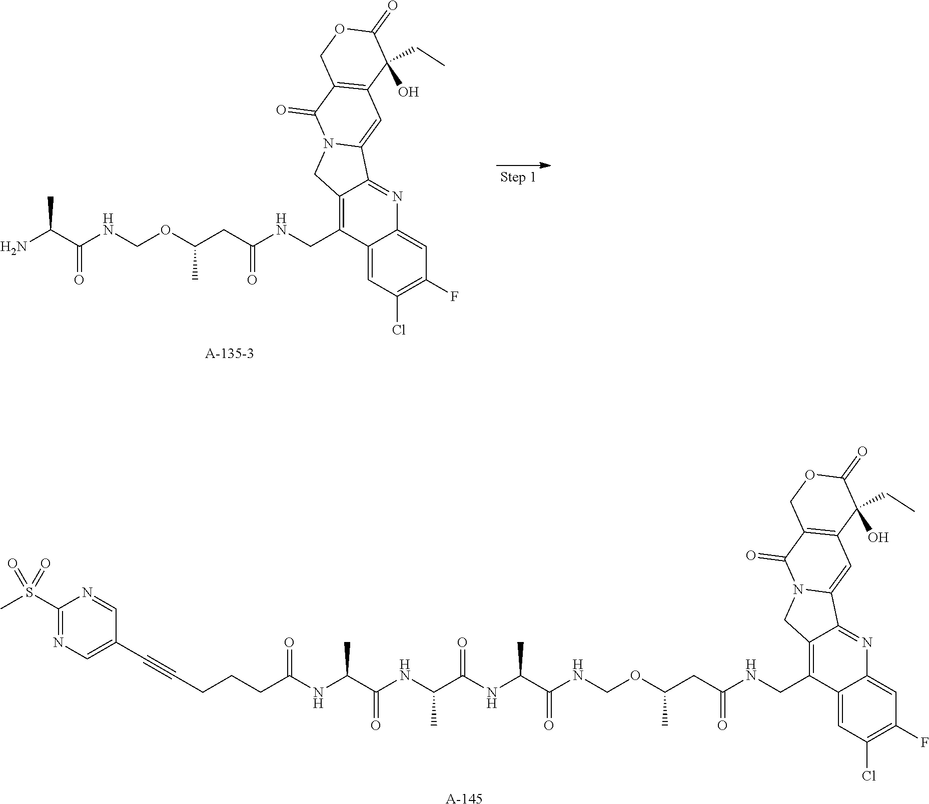 [SMILES: CC[C@@]1(O)C(=O)OCc2c1cc1n(c2=O)Cc2c-1nc1cc(F)c(Cl)cc1c2CNC(=O)C[C@H](C)OCNC(=O)[C@H](C)N ; CC[C@@]1(O)C(=O)OCc2c1cc1n(c2=O)Cc2c-1nc1cc(F)c(Cl)cc1c2CNC(=O)C[C@H](C)OCNC(=O)[C@H](C)NC(=O)[C@H](C)NC(=O)[C@H](C)NC(=O)CCCC#Cc1cnc(S(C)(=O)=O)nc1 ; "A-145"]

(S)-3-(((S)-2-Aminopropionamido)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)butanamide (25 mg, 0.041 mmol) and (6-(2-(methylsulfonyl)pyrimidin-5-yl)hex-5-ynoyl)-L-alanyl-L-alanine (18.3 mg, 0.045 mmol) were dissolved in DMF (1 mL), added with HATU (30.9 mg, 0.082 mmol) and DIPEA (15.7 mg, 0.12 mmol), and reacted at 25° C. for 1 hour. The reaction solution was directly purified by preparative high performance liquid chromatography and freeze-dried to obtain the title compound (13.1 mg, 0.013 mmol).

Its structural characterization data are as follows:

MS m/z (ESI): 1008.3[M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 30 | 70 | 32 |
| 4.00 | 30 | 70 | 32 |
| 24.00 | 90 | 10 | 32 |

### Example 41: Preparation of N-((5R,10S,13S,16S)-1-((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)-5,10,13-trimethyl-3,9,12,15-tetraoxo-6-oxa-2,8,11,14-tetraazaheptadecan-16-yl)-6-(2-(methylsulfonyl)pyrimidin-5-yl)hex-5-ynamide (A-146)

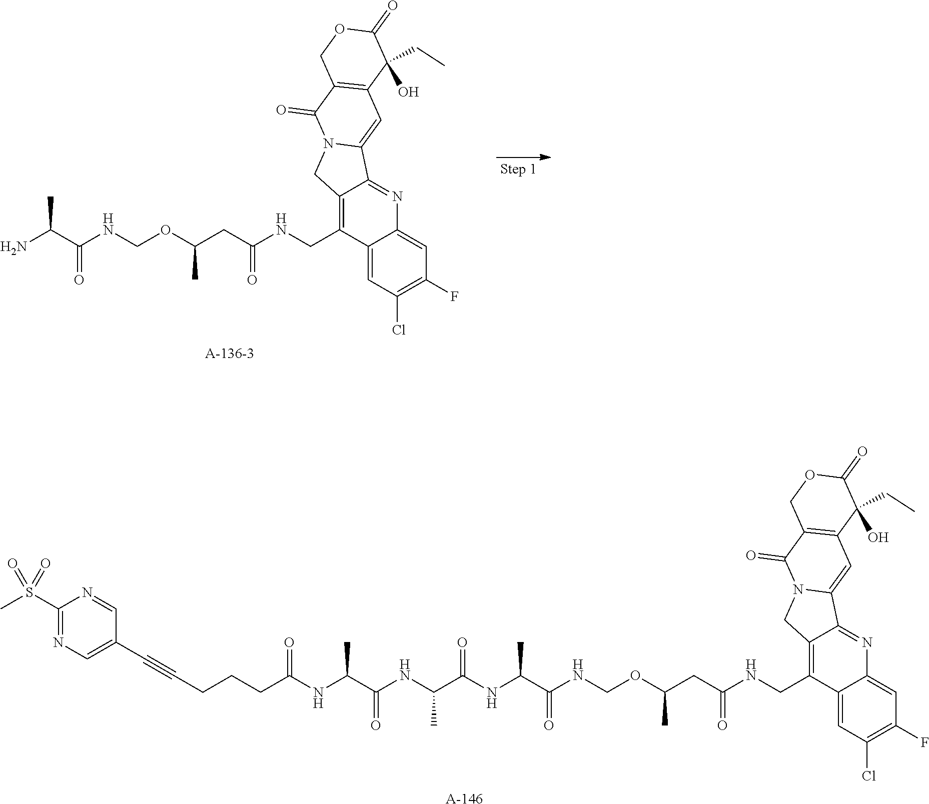 [SMILES: CC[C@@]1(O)C(=O)OCc2c1cc1n(c2=O)Cc2c-1nc1cc(F)c(Cl)cc1c2CNC(=O)C[C@@H](C)OCNC(=O)[C@H](C)N ; CC[C@@]1(O)C(=O)OCc2c1cc1n(c2=O)Cc2c-1nc1cc(F)c(Cl)cc1c2CNC(=O)C[C@@H](C)OCNC(=O)[C@H](C)NC(=O)[C@H](C)NC(=O)[C@H](C)NC(=O)CCCC#Cc1cnc(S(C)(=O)=O)nc1 ; "A-146"]

(R)-3-(((S)-2-Aminopropionamido)methoxy)-N—(((S)-9-chloro-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)butanamide (20 mg, 30.21 μmol) and DIPEA (19.52 mg, 151.04 μmol) were dissolved in DMF (4 mL), reacted under stirring for 10 minutes, then (6-(2-(methylsulfonyl)pyrimidin-5-yl) hex-5-ynyl)-L-alanine-L-alanine (12.40 mg, 30.21 μmol) and HATU (22.97 mg, 60.42 μmol) were added to the reaction system and stirred at 25° C. for 2 hours. The reaction solution was directly purified by preparative high performance liquid chromatography and freeze-dried to obtain the title compound (14 mg, 13.60 μmol).

Its structural characterization data are as follows:

MS m/z (ESI): 1008.6 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 20 | 80 | 30 |
| 2.00 | 20 | 80 | 30 |
| 18.00 | 90 | 10 | 30 |

### Example 42: Preparation of 4-((S)-2-(4-aminobutyl)-35-(4-((6-(2-(methylsulfonyl) pyrimidin-5-yl)hex-5-ynamido)methyl)-1H-1,2,3-triazol-1-yl)-4,8-dioxo-6,12,15,18,21,24,27,30,33-nonaoxa-3,9-diazapentatriacontanamido)benzyl ((R)-11-(((R)-2-cyclopropyl-2-hydroxyacetamido)methyl)-4-ethyl-8-fluoro-9-methyl-3,14-dioxo-3,4,12,14-tetrahydro-1H-pyrano[3′,4′:6,7]indolizino[1,2-b]quinolin-4-yl)carbonate (A-151)

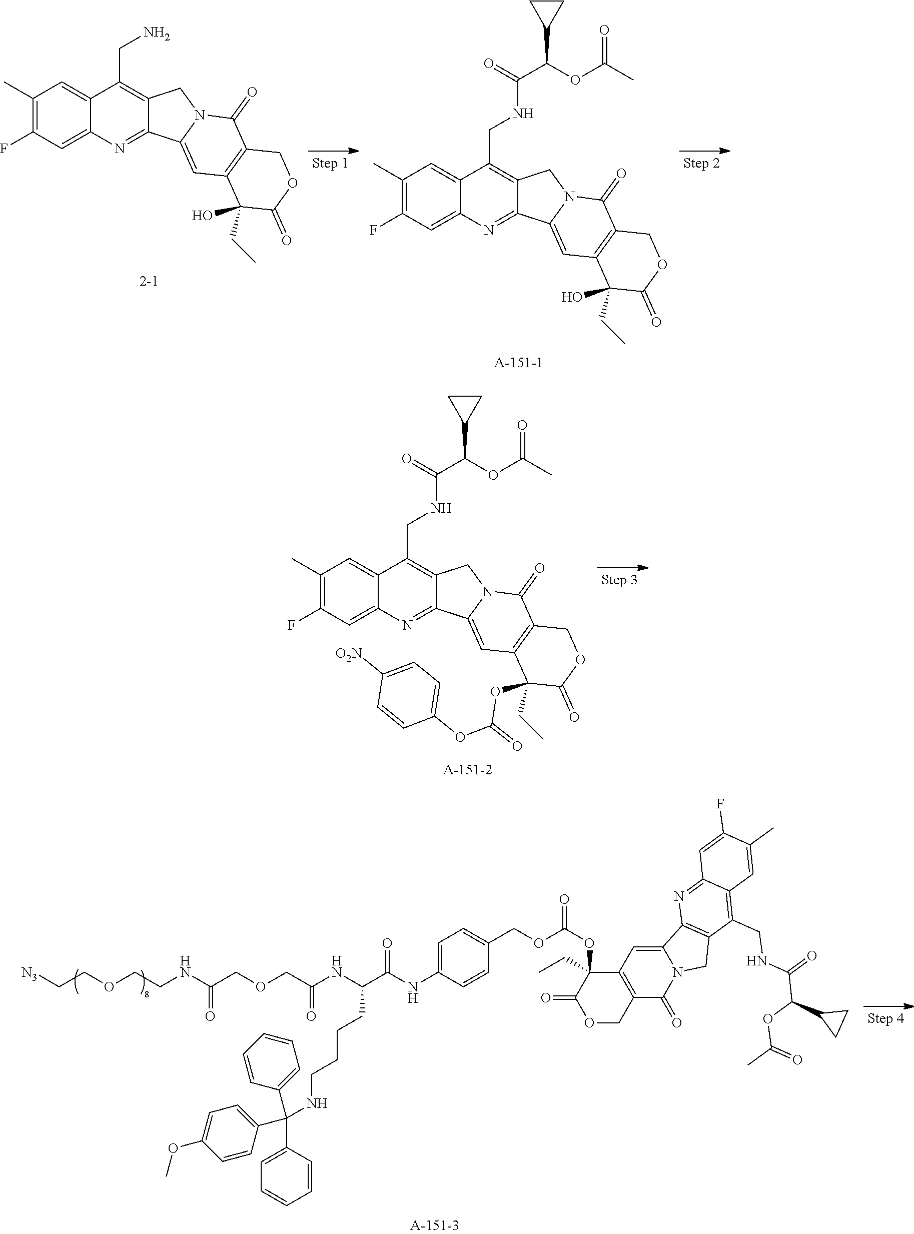

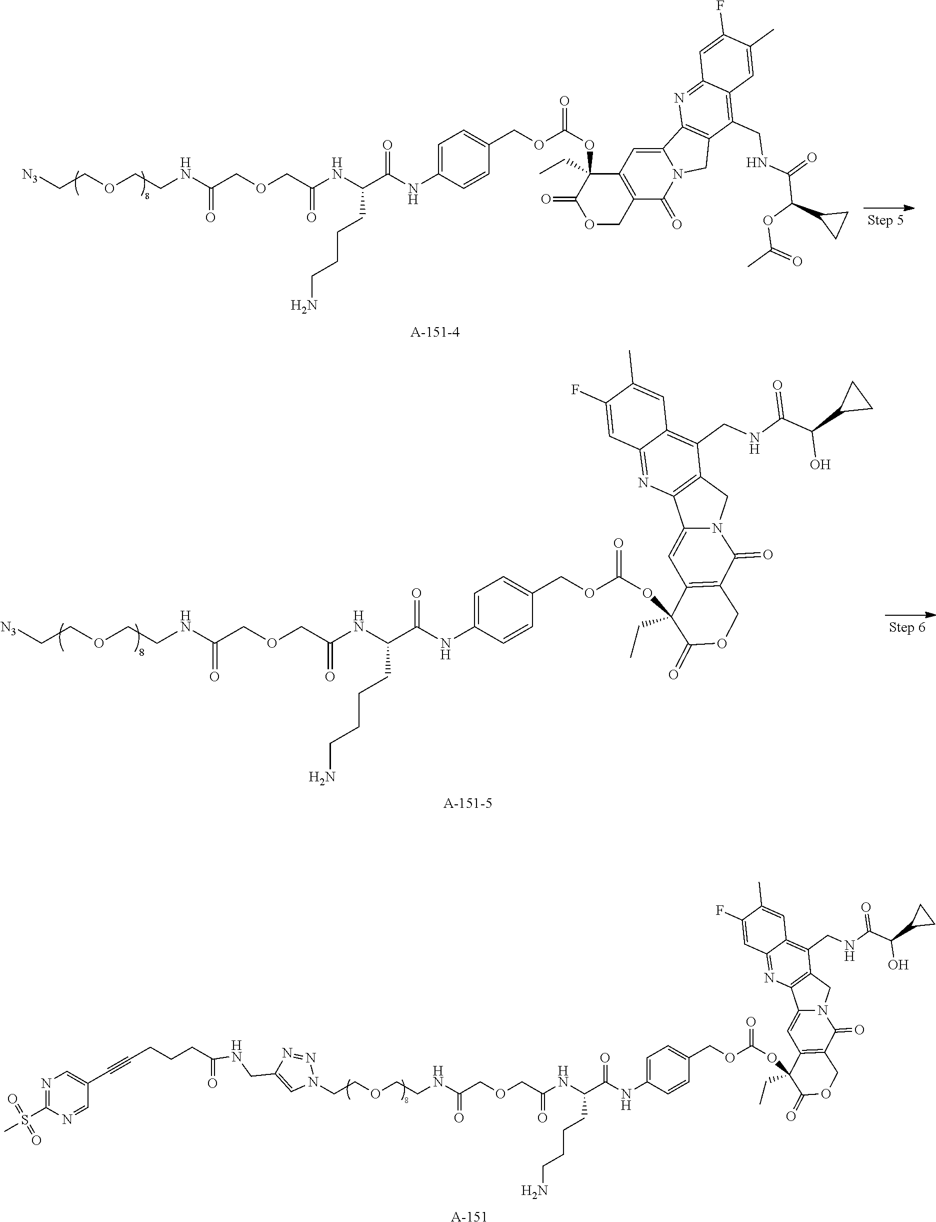

### Step 1: Preparation of (R)-1-cyclopropyl-2-((((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)amino)-2-oxoethyl acetate (A-151-1)

(R)-2-Cyclopropyl-2-hydroxyacetic acid (425.42 mg, 3.66 mmol) was dissolved in acetic anhydride (5 mL), heated to 40° C. and reacted for 2 hours, and evaporated under reduced pressure to remove solvent to obtain an oily product. The oily product was dissolved in dry DMF (10 mL), added with (S)-11-(aminomethyl)-4-ethyl-8-fluoro-4-hydroxy-9-methyl-1,12-dihydro-14H-pyrano[3′,4′:6,7]indolizino[1,2-b]quinolin-3,14(4H)-dione (500 mg, 1.22 mmol), then added dropwise with DIPEA (315.67 mg, 2.44 mmol), and stirred continuously for 2 hours. The reaction solution was added dropwise with dilute hydrochloric acid for neutralization, purified by flash column chromatography (C18, water/acetonitrile=0.35), and freeze-dried to obtain the title compound (363 mg, 0.66 mmol).

Its structural characterization data are as follows:

MS m/z (ESI): 549.6 [M+H]⁺

### Step 2: Preparation of (R)-1-cyclopropyl-2-((((S)-4-ethyl-8-fluoro-9-methyl-4-(((4-nitrophenoxy)carbonyl)oxy)-3,14-dioxo-3,4,12,14-tetrahydro-1H-pyrano[3′,4′:6,7]indolizino[1,2-b]quinolin-11-yl)methyl)amino)-2-oxoethyl acetate (A-151-2)

(R)-1-Cyclopropyl-2-((((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)amino)-2-oxoethyl acetate (390 mg, 0.71 mmol) and DMAP (216.75 mg, 1.77 mmol) were dissolved in dry dichloromethane (40 mL), added with p-nitrophenyl chloroformate (357.61 mg, 1.77 mmol) under stirring, and reacted under stirring and nitrogen protection for 3 hours. LCMS monitoring showed that a small amount of raw material remained. The reaction solution was concentrated under reduced pressure, added with ethyl acetate for slurrying, and filtered, and the solid was washed with a small amount of ethyl acetate and dried to obtain the title compound (500 mg, 0.70 mmol).

Its structural characterization data are as follows:

MS m/z (ESI): 714.6 [M+H]⁺

### Step 3: Preparation of (R)-2-((((S)-4-((((4-((S)-35-azido-2-(4-(((4-methoxyphenyl) diphenylmethyl)amino)butyl)-4,8-dioxo-6,12,15,18,21,24,27,30,33-nonaoxa-3,9-diazapentatriacontanamido)benzyl)oxy)carbonyl)oxy)-4-ethyl-8-fluoro-9-methyl-3,14-dioxo-3,4,12,14-tetrahydro-1H-pyrano[3′,4′:6,7]indolizino[1,2-b]quinolin-11-yl)methyl)amino)-1-cyclopropyl-2-oxoethyl acetate (A-151-3)

(R)-1-Cyclopropyl-2-((((S)-4-ethyl-8-fluoro-9-methyl-4-(((4-nitrophenoxy)carbonyl)oxy)-3,14-dioxo-3,4,12,14-tetrahydro-1H-pyrano[3′,4′:6,7]indolizino[1,2-b]quinolin-11-yl)methyl)amino)-2-oxoethyl acetate (500 mg, 0.7 mmol), (S)-2-(32-azido-5-oxo-3,9,12,15,18,21,24,27,30-nonaoxa-6-azadotriacontanamido)-N-(4-(hydroxymethyl)phenyl)-6-(((4-methoxyphenyl)diphenylmethyl)amino)hexanamide (593.43 mg, 0.56 mmol) and DMAP (256.43 mg, 2.10 mmol) were dissolved in dry dichloromethane (15 mL), and reacted under stirring for 3 hours. The title compound (674 mg, 0.41 mmol) was obtained by silica gel column purification (0-30% methanol/dichloromethane).

Its structural characterization data are as follows:

MS m/z (ESI): 1635.7 [M+H]⁺

### Step 4: Preparation of 4-((S)-2-(4-aminobutyl)-35-azido-4,8-dioxo-6,12,15,18,21,24,27,30,33-nonaoxa-3,9-diazapentatriacontanamido)benzyl((R)-9-ethyl-4-hydroxy-10,13-dioxo-2,3,9,10,13,15-hexahydro-1H,12H-benzo[de]pyrano[3′,4′:6,7]indolizino[1,2-b]quinolin-9-yl)carbonate (A-155-4)

(R)-2-((((S)-4-((((4-((S)-35-Azido-2-(4-(((4-methoxyphenyl)diphenylmethyl)amino)butyl)-4,8-dioxo-6,12,15,18,21,24,27,30,33-nonaoxa-3,9-diazapentatriacontanamido)benzyl)oxy)carbonyl)oxy)-4-ethyl-8-fluoro-9-methyl-3,14-dioxo-3,4,12,14-tetrahydro-1H-pyrano[3′,4′:6,7]indolizino[1,2-b]quinolin-11-yl)methyl)amino)-1-cyclopropyl-2-oxoethyl acetate (674 mg, 0.41 mmol) was dissolved in dichloromethane (10 mL), added dropwise with TFA (0.5 mL) under stirring, and reacted continuously for 1 hour. The crude product was concentrated under reduced pressure and directly subjected to the reaction in the next step.

Its structural characterization data are as follows:

MS m/z (ESI): 1363.7 [M+H]⁺

### Step 5: Preparation of 4-((S)-2-(4-aminobutyl)-35-azido-4,8-dioxo-6,12,15,18,21,24,27,30,33-nonaoxa-3,9-diazapentatriacontanamido)benzyl ((S)-11-(((R)-2-cyclopropyl-2-hydroxyacetamido)methyl)-4-ethyl-8-fluoro-9-methyl-3,14-dioxo-3,4,12,14-tetrahydro-1H-pyrano[3′,4′:6,7]indolizino[1,2-b]quinolin-4-yl)carbonate (A-151-5)

(R)-2-((((S)-4-((((4-((S)-2-(4-Aminobutyl)-35-azido-4,8-dioxo-6,12,15,18,21,24,27,30,33-nonaoxa-3,9-diazapentatriacontanamido)benzyl)oxy)carbonyl)oxy)-4-ethyl-8-fluoro-9-methyl-3,14-dioxo-3,4,12,14-tetrahydro-1H-pyrano[3′,4′:6,7]indolizino[1,2-b]quinolin-11-yl)methyl)amino)-1-cyclopropyl-2-oxoethyl acetate (560 mg crude, 0.41 mmol) was dissolved in MeOH (6 mL), added dropwise with sodium carbonate (87.06 mg, 0.82 mmol) in aqueous solution (0.6 mL) under stirring, and reacted under stirring for 1 hour. The reaction solution was purified by flash column chromatography (C18, water/acetonitrile=0.26) and freeze-dried to obtain the title compound (124 mg, 0.09 mmol).

Its structural characterization data are as follows:

MS m/z (ESI): 1321.4 [M+H]⁺

### Step 6: Preparation of 4-((S)-2-(4-aminobutyl)-35-(4-((6-(2-(methylsulfonyl)pyrimidin-5-yl) hex-5-ynamido)methyl)-1H-1,2,3-triazol-1-yl)-4,8-dioxo-6,12,15,18,21,24,27,30,33-nonaoxa-3,9-diazapentatriacontanamido)benzyl ((R)-11-(((R)-2-cyclopropyl-2-hydroxyacetylamino)methyl)-4-ethyl-8-fluoro-9-methyl-3,14-dioxo-3,4,12,14-tetrahydro-1H-pyrano[3′,4′:6,7]indolizino[1,2-b]quinolin-4-yl)carbonate (A-151)

6-(2-Methylsulfonylpyrimidin-5-yl)-N-propyl-2-ynylhexan-5-amide (5.31 mg, 17.40 mol), 4-((S)-2-(4-aminobutyl)-35-azido-4,8-dioxo-6,12,15,18,21,24,27,30,33-nonaoxa-3,9-diazapentatriacontanamido)benzyl((R)-9-ethyl-4-hydroxy-10,13-dioxo-2,3,9,10,13,15-hexahydro-1H,12H-benzo[de]pyrano[3′,4′:6,7]indolizino[1,2-b]quinolin-9-yl)carbonate (22 mg, 17.40 μmol) and CuBr (2.50 mg, 17.40 μmol) were dissolved in DMSO (1 mL) and water (0.1 mL), and reacted under stirring for 1 hour. The reaction solution was filtered, purified by preparative high performance liquid chromatography and freeze-dried to obtain the target compound (6.08 mg, 3.80 μmol).

Its structural characterization data are as follows:

MS m/z (ESI): 1626.7[M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 28 |
| 2.00 | 10 | 90 | 28 |
| 20.00 | 90 | 10 | 28 |

### Example 43: Preparation of N-((31S,34S)-35-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-31-isopropyl-3,6,9,12,15,18,21,24,27,34-decamethyl-2,5,8,11,14,17,20,23,26,29,32,35-dodecaoxo-3,6,9,12,15,18,21,24,27,30,33-undecazapentatriacontyl)-N-methyl-6-(2-(methylsulfonyl)pyrimidin-5-yl)hex-5-ynamide (A-157)

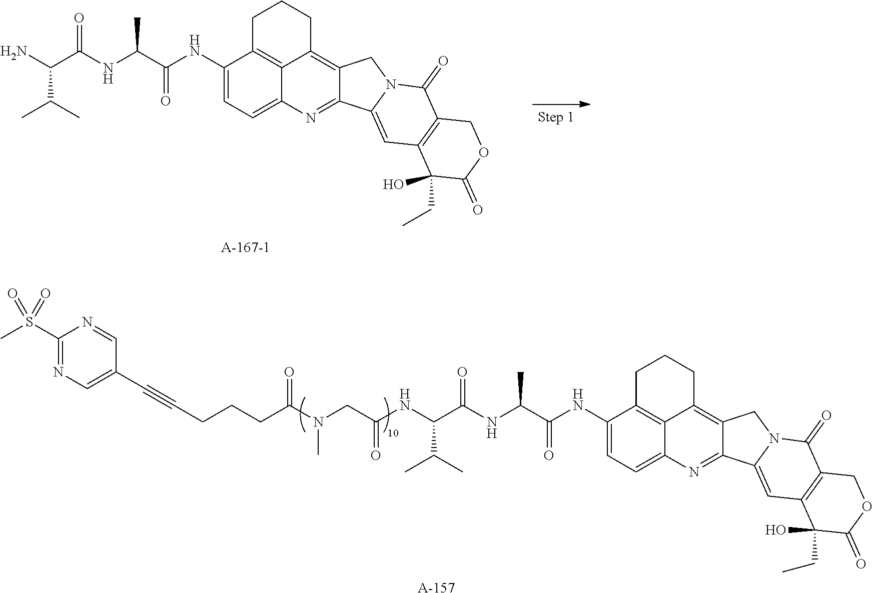

(S)-2-Amino-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropane-2-yl)-3-methylbutanamide (15.0 mg, 0.026 mmol) and INT-1 (38.5 mg, 0.039 mmol) were dissolved in DMF (2 mL), added with 2-(7-azobenzotriazole)-N,N,N′,N′-tetramethyluronium hexafluorophosphate (20.1 mg, 0.05 mmol) and DIPEA (16.9 mg, 0.13 mmol), and stirred at room temperature for 1 hour. After the reaction was completed, the reaction solution was directly purified by preparative high performance liquid chromatography and freeze-dried to obtain the title compound (14.8 mg, 9.82 μmol).

Its structural characterization data are as follows:

MS m/z (ESI): 1506.3 [M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% trifluoroacetic acid)

| Time | Mobile phase A | Mobile phase B | Flow rate |
|---|---|---|---|
| [min] | [%] | [%] | [mL/min] |
| 0.00 | 5 | 95 | 30 |
| 3.00 | 5 | 95 | 30 |
| 18.00 | 70 | 30 | 30 |

### Example 44: Preparation of N—((S)-1-(((S)-6-amino-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxohex-2-yl)amino)-3-methyl-1-oxobutan-2-yl)-6-(2-(methylsulfonyl)pyrimidin-5-yl)hex-5-ynamide (A-160)

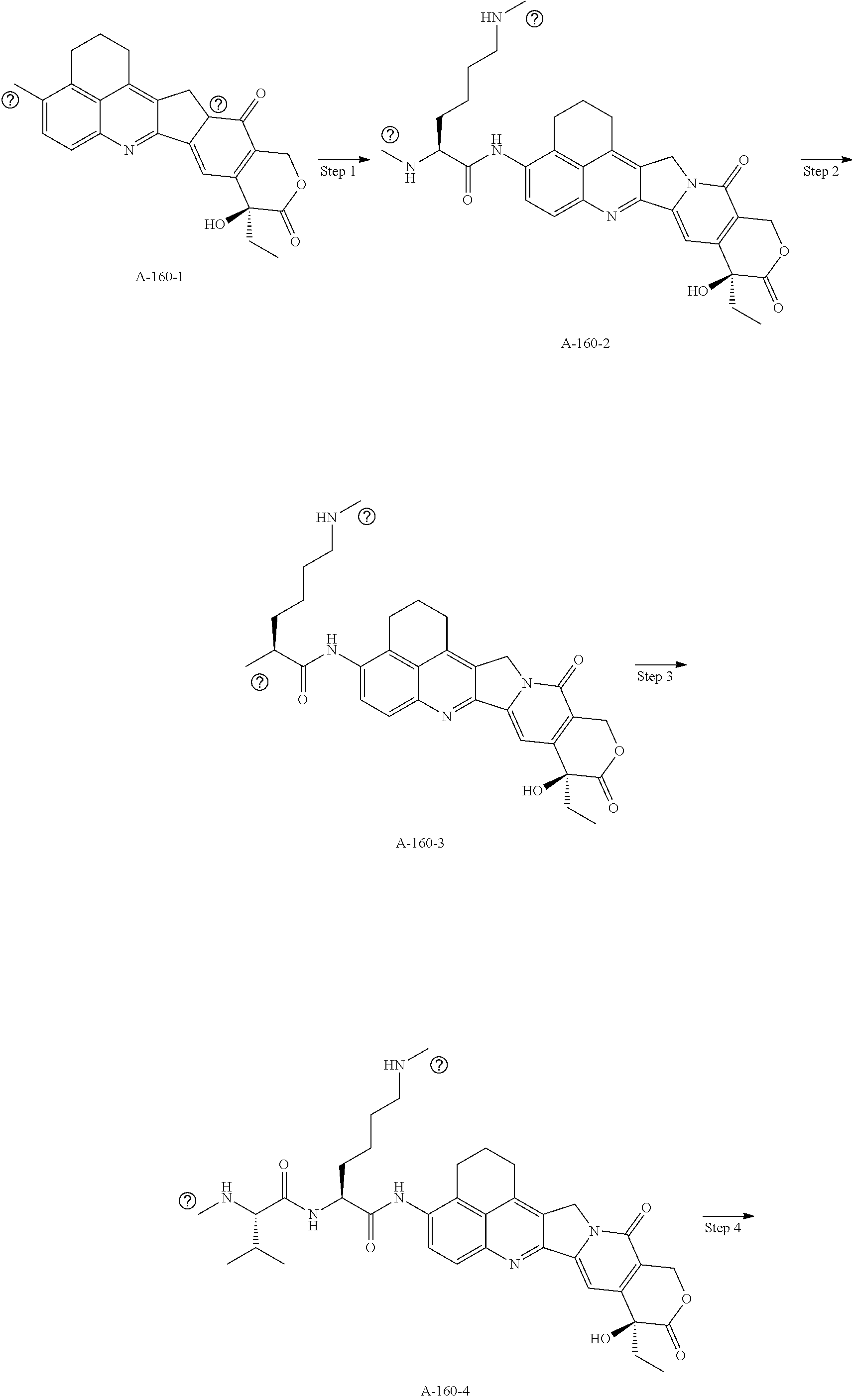 [SMILES: CC[C@@]1(O)C(=O)OCC2=C1C=C1c3nc4ccc(C)c5c4c(c3CC1C2=O)CCC5 ; CC[C@@]1(O)C(=O)OCc2c1cc1n(c2=O)Cc2c-1nc1ccc(NC(=O)[C@H](CCCCNC)NC)c3c1c2CCC3 ; CC[C@@]1(O)C(=O)OCc2c1cc1n(c2=O)Cc2c-1nc1ccc(NC(=O)[C@@H](C)CCCCNC)c3c1c2CCC3 ; CC[C@@]1(O)C(=O)OCc2c1cc1n(c2=O)Cc2c-1nc1ccc(NC(=O)[C@H](CCCCNC)NC(=O)[C@@H](NC)C(C)C)c3c1c2CCC3]

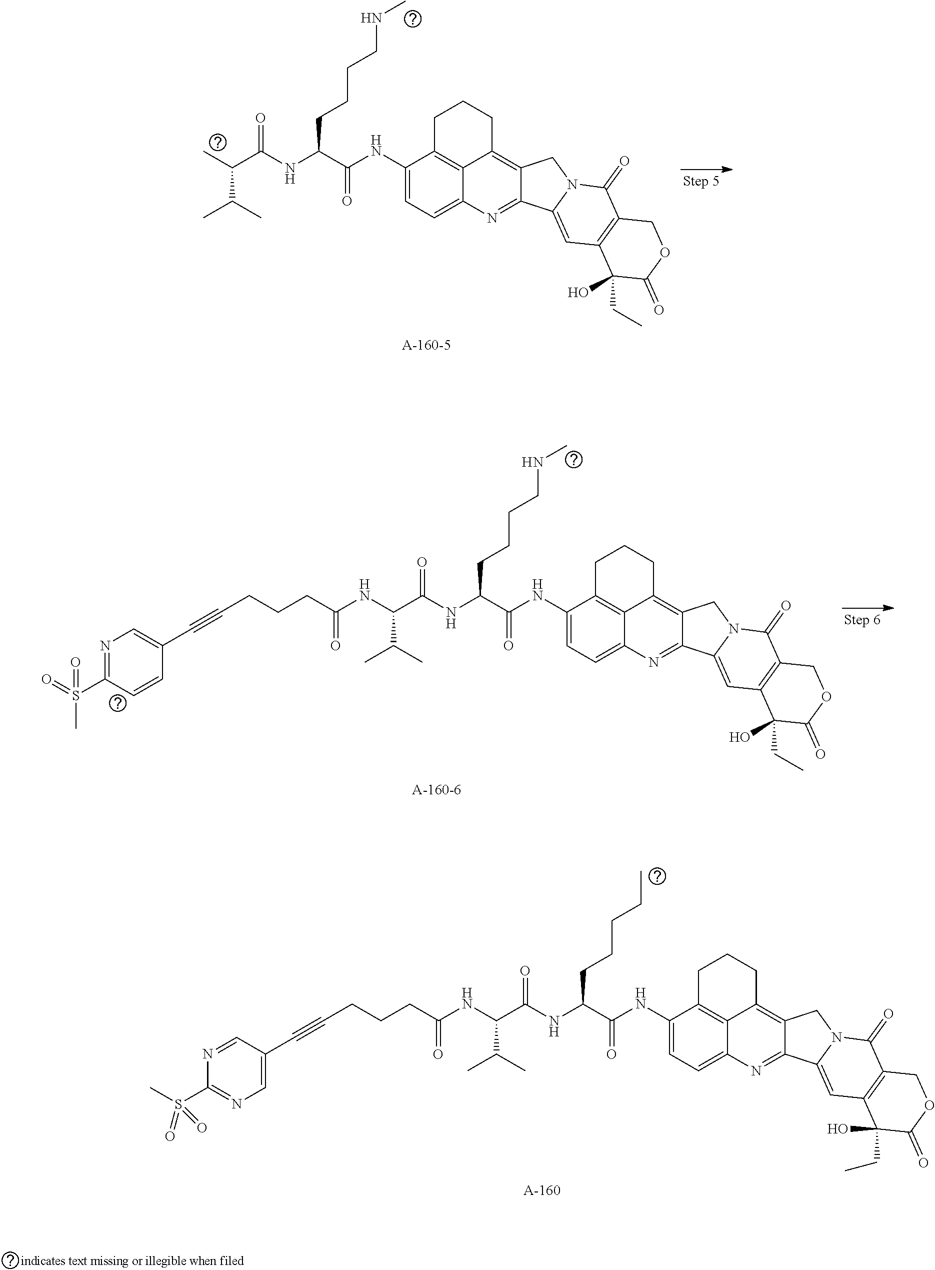 [SMILES: CC[C@@]1(O)C(=O)OCc2c1cc1n(c2=O)Cc2c-1nc1ccc(NC(=O)[C@H](CCCCNC)NC(=O)[C@@H](C)C(C)C)c3c1c2CCC3 ; CC[C@@]1(O)C(=O)OCc2c1cc1n(c2=O)Cc2c-1nc1ccc(NC(=O)[C@H](CCCCNC)NC(=O)[C@@H](NC(=O)CCCC#Cc3ccc(S(C)(=O)=O)nc3)C(C)C)c3c1c2CCC3 ; CCCCC[C@H](NC(=O)[C@@H](NC(=O)CCCC#Cc1cnc(S(C)(=O)=O)nc1)C(C)C)C(=O)Nc1ccc2nc3c(c4c2c1CCC4)Cn1c-3cc2c(c1=O)COC(=O)[C@]2(O)CC ; "A-160"]

### Step 1: Preparation of (9H-fluoren-9-yl)methyl tert-butyl ((5′)-6-(((5′)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexan-1,5-diyl)dicarbamate (A-160-2)

N²-(((9H-Fluoren-9-yl)methoxy)carbonyl)-N-(tert-butyloxycarbonyl)-L-lysine (116.14 mg, 0.25 mmol), (5)-4-amino-9-ethyl-9-hydroxy-1,2,3,9,12,15-hexahydro-10H,13H-benzo[de]pyrano[3′,4′:6,7]indolizino[1,2-b]quinolin-10,13-dione (50 mg, 0.12 mmol) and HATU (94.19 mg, 0.25 mmol) were dissolved in dry DMF (1 mL), added dropwise with DIPEA (48.05 mg, 0.37 mmol), and reacted under stirring and nitrogen protection for 12 hours. The reaction solution was added dropwise with 0.5N hydrochloric acid aqueous solution to neutralize the reaction, and solid precipitated. The solid was filtered, washed with water, and dried to obtain a crude product (100 mg, 0.11 mmol).

Its structural characterization data are as follows:

MS m/z (ESI): 853.9[M+H]⁺

### Step 2: Preparation of tert-butyl ((S)-5-amino-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (A-160-3)

(9H-Fluoren-9-yl)methyl tert-butyl ((S)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexan-1,5-diyl)dicarbamate (100 mg, 0.11 mmol) was dissolved in DMF (1 mL), added dropwise with diethylamine (0.1 mL), and stirred for 0.5 hours. The reaction solution was purified by flash column chromatography (C18, water/acetonitrile=2/1) and freeze-dried to obtain the title compound (45 mg, 0.06 mmol).

Its structural characterization data are as follows:

MS m/z (ESI): 632.4[M+H]⁺

### Step 3: Preparation of tert-butyl ((S)-5-((S)-2-((((9H-fluoren-9-yl)methoxy)carbonyl) amino)-3-methylbutanamido)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (A-160-4)

Fmoc-L-valine (12.02 mg, 35.41 μmol), tert-butyl ((S)-5-amino-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (20 mg, 29.51 μmol) and HATU (13.63 mg, 35.88 μmol) were dissolved in DMF (1 mL), added dropwise with DIPEA (11.44 mg, 88.53 μmol), and reacted under stirring for 1 hour. The reaction solution was added with water to precipitate solid, which was filtered, washed with water and dried to obtain the crude product (28 mg, 29.38 μmol).

Its structural characterization data are as follows:

MS m/z (ESI): 954.5[M+H]⁺

### Step 4: Preparation of tert-butyl ((S)-5-((S)-2-amino-3-methylbutanamido)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (A-160-5)

Tert-butyl ((S)-5-((S)-2-((((9H-fluoren-9-yl)methoxy)carbonyl)amino)-3-methyl-butanamido)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (28 mg, 29.38 μmol) was dissolved in DMF (1 mL), added dropwise with diethylamine (0.1 mL), and reacted under stirring for 0.5 hours. The reaction solution was subjected to flash column chromatography (C18, water/acetonitrile=2/1) and freeze-dried to obtain the title compound (15 mg, 20.52 μmol).

Its structural characterization data are as follows:

MS m/z (ESI): 731.4[M+H]⁺

### Step 5: Preparation of tert-butyl ((S)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-((S)-3-methyl-2-(6-(2-(methylsulfonyl)pyrimidin-5-yl)hex-5-ynamido)butanamido)-6-oxohexyl)carbamate (A-160-6)

2,5-Dioxopyrrolidin-1-yl 6-(2-(methylsulfonyl)pyrimidin-5-yl)hex-5-ynoate (7.05 mg, 19.31 μmol) and tert-butyl ((S)-5-((S)-2-amino-3-methylbutanamido)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (15 mg, 19.31 μmol) were dissolved in DMF (1 mL), added dropwise with DIPEA (2.50 mg, 19.31 μmol), and reacted under stirring for 1 hour. The reaction solution was directly purified by preparative high performance liquid chromatography and freeze-dried to obtain the title compound (16 mg, 16.31 μmol).

Its structural characterization data are as follows:

MS m/z (ESI): 981.2[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 20.00 | 90 | 10 | 28 |

### Step 6: Preparation of N—((S)-1-(((S)-6-amino-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxohexan-2-yl)amino)-3-methyl-1-oxobutan-2-yl)-6-(2-(methylsulfonyl)pyrimidin-5-yl)hex-5-ynamide (A-160)

Tert-butyl ((S)-6-(((S)-9-ethyl-9-hydroxy-10,13-dioxo-2,3,9,10,13,15-hexahydro-1H,12H-benzo[de]pyrano[3′,4′: 6,7]indolizino[1,2-b]quinolin-4-yl)amino)-5-((S)-3-methyl-2-(6-(2-(methylsulfonyl)pyrimidin-5-yl)hex-5-ynamido)butanamido)-6-oxohexyl)carbamate (16 mg, 16.31 μmol) was dissolved in dichloromethane (2 mL), added dropwise with trifluoroacetic acid (0.5 mL) under stirring, and reacted continuously for 1 hour. The reaction solution was concentrated under reduced pressure, purified by preparative high performance liquid chromatography, and freeze-dried to obtain the title compound (7.21 mg, 7.17 μmol).

Its structural characterization data are as follows:

MS m/z (ESI): 881.3[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 10 | 90 | 28 |
| 4.00 | 10 | 90 | 28 |
| 24.00 | 90 | 10 | 28 |

### Example 45: Preparation of N-(2-(((S)-6-amino-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxohexan-2-yl)amino)-2-oxoethyl)-6-(2-(methylsulfonyl)pyrimidin-5-yl)hex-5-ynamide (A-161)

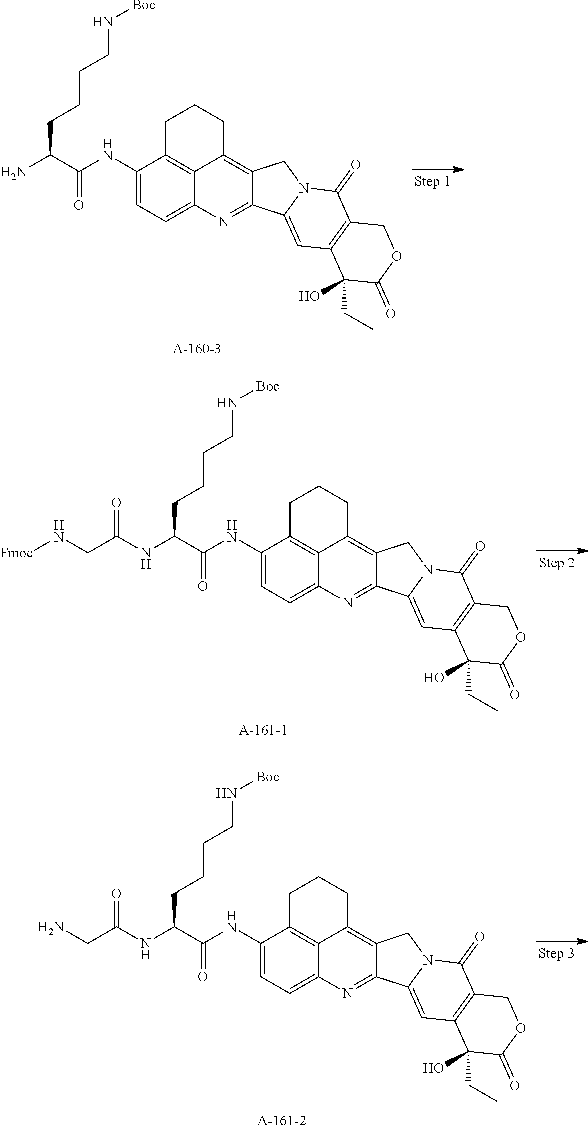

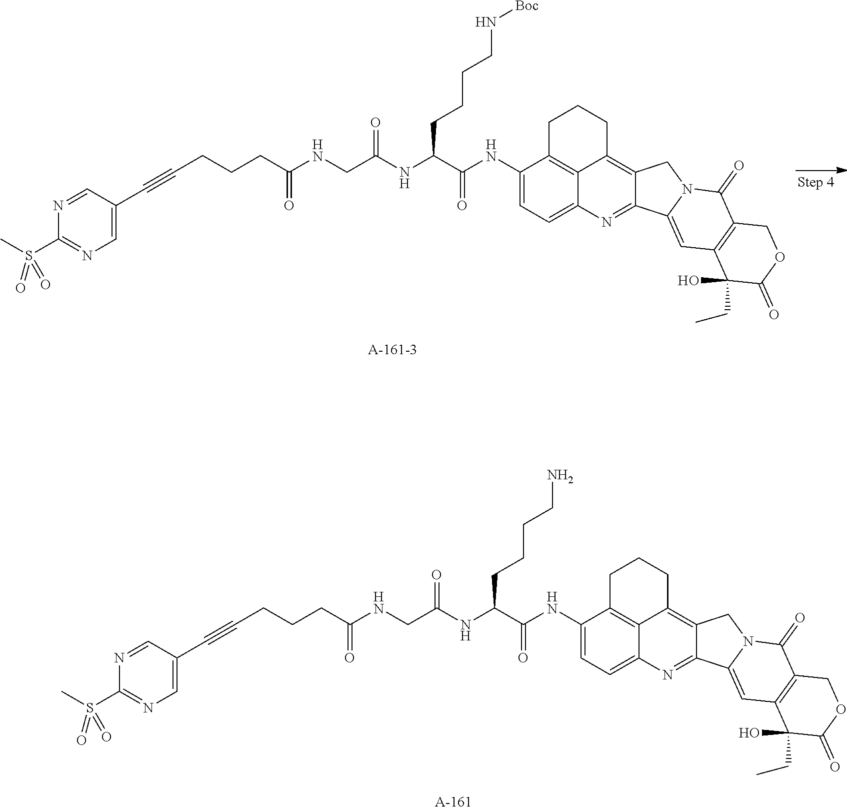 [SMILES: CC[C@@]1(O)C(=O)OCc2c1cc1n(c2=O)Cc2c-1nc1ccc(NC(=O)[C@H](CCCCNC(=O)OC(C)(C)C)NC(=O)CNC(=O)CCCC#Cc3cnc(S(C)(=O)=O)nc3)c3c1c2CCC3 ; CC[C@@]1(O)C(=O)OCc2c1cc1n(c2=O)Cc2c-1nc1ccc(NC(=O)[C@H](CCCCN)NC(=O)CNC(=O)CCCC#Cc3cnc(S(C)(=O)=O)nc3)c3c1c2CCC3 ; "A-161"]

### Step 1: Preparation of tert-butyl ((S)-5-(2-((((9H-fluoren-9-yl)methoxy)carbonyl)amino)acetamido)-6-(((S)-9-ethyl-9-hydroxy-10,13-dioxo-2,3,9,10,13,15-hexahydro-1H,12H-benzo[de]pyrano[13′,4′:6,7]indolizino[1,2-b]quinolin-4-yl)amino)-6-oxohexyl)carbamate (A-161-1)

Fmoc-glycine (10.53 mg, 35.41 μmol), tert-butyl ((S)-5-amino-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (20 mg, 29.51 μmol) and HATU (13.63 mg, 35.88 μmol) were dissolved in DMF (1 mL), added dropwise with DIPEA (11.44 mg, 88.53 μmol), and reacted under stirring for 1 hour. The reaction solution was added with water to precipitate solid, which was filtered, washed with water, and dried to obtain the crude product (26 mg, 28.54 μmol).

Its structural characterization data are as follows:

MS m/z (ESI): 912.5[M+H]⁺

### Step 2: Preparation of tert-butyl ((S)-5-(2-aminoacetamido)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (A-161-2)

Tert-butyl ((S)-5-(2-((((9H-fluoren-9-yl)methoxy)carbonyl)amino)acetamido)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (26 mg, 28.54 μmol) was dissolved in DMF (1 mL), added dropwise with diethylamine (0.1 mL), and reacted under stirring for 0.5 hours. The reaction solution was subjected to flash column chromatography (C18, water/acetonitrile=2/1), and then freeze-dried to obtain the title compound (15 mg, 20.52 μmol).

Its structural characterization data are as follows:

MS m/z (ESI): 689.3[M+H]⁺

### Step 3: Preparation of tert-butyl ((S)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-(2-(6-(2-(methylsulfonyl)pyrimidin-5-yl)hex-5-ynamido)-6-oxohexyl)carbamate (A-161-3)

2,5-Dioxopyrrolidin-1-yl 6-(2-(methylsulfonyl)pyrimidin-5-yl)hex-5-ynoate (8.95 mg, 24.50 mol) and tert-butyl ((S)-5-(2-aminoacetamido)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-6-oxohexyl)carbamate (15 mg, 20.41 μmol) were dissolved in DMF (1 mL), added dropwise with DIPEA (5.28 mg, 40.83 μmol), and reacted under stirring for 1 hour. The reaction solution was directly purified by preparative high performance liquid chromatography and freeze-dried to obtain the title compound (15 mg, 15.97 μmol).

Its structural characterization data are as follows:

MS m/z (ESI): 939.4[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 4.00 | 30 | 70 | 28 |
| 24.00 | 90 | 10 | 28 |

### Step 4: Preparation of N-(2-(((S)-6-amino-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxohexan-2-yl)amino)-2-oxoethyl)-6-(2-(methylsulfonyl)pyrimidin-5-yl)hex-5-ynamide (A-161)

Tert-butyl ((S)-6-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-(2-(6-(2-(methylsulfonyl)pyrimidin-5-yl)hex-5-ynamido)-6-oxohexyl)carbamate (15 mg, 15.97 μmol) was dissolved in DCM (2 mL), added dropwise with trifluoroacetic acid (0.5 mL) under stirring, and reacted continuously for 1 hour. The reaction solution was concentrated under reduced pressure, purified by preparative high performance liquid chromatography, and freeze-dried to obtain the title compound (6.96 mg, 7.23 μmol).

Its structural characterization data are as follows:

MS m/z (ESI): 839.4[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% trifluoroacetic acid)

| Time | Mobile phase A | Mobile phase B | Flow rate |
|---|---|---|---|
| [min] | [%] | [%] | [mL/min] |
| 0.00 | 10 | 90 | 28 |
| 4.00 | 10 | 90 | 28 |
| 24.00 | 90 | 10 | 28 |

### Example 46: N—((S)-1-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-1-oxopropan-2-yl)amino)-1-oxopropan-2-yl)-6-(2-(methylsulfonyl)pyrimidin-5-yl)hex-5-ynamide (A-163)

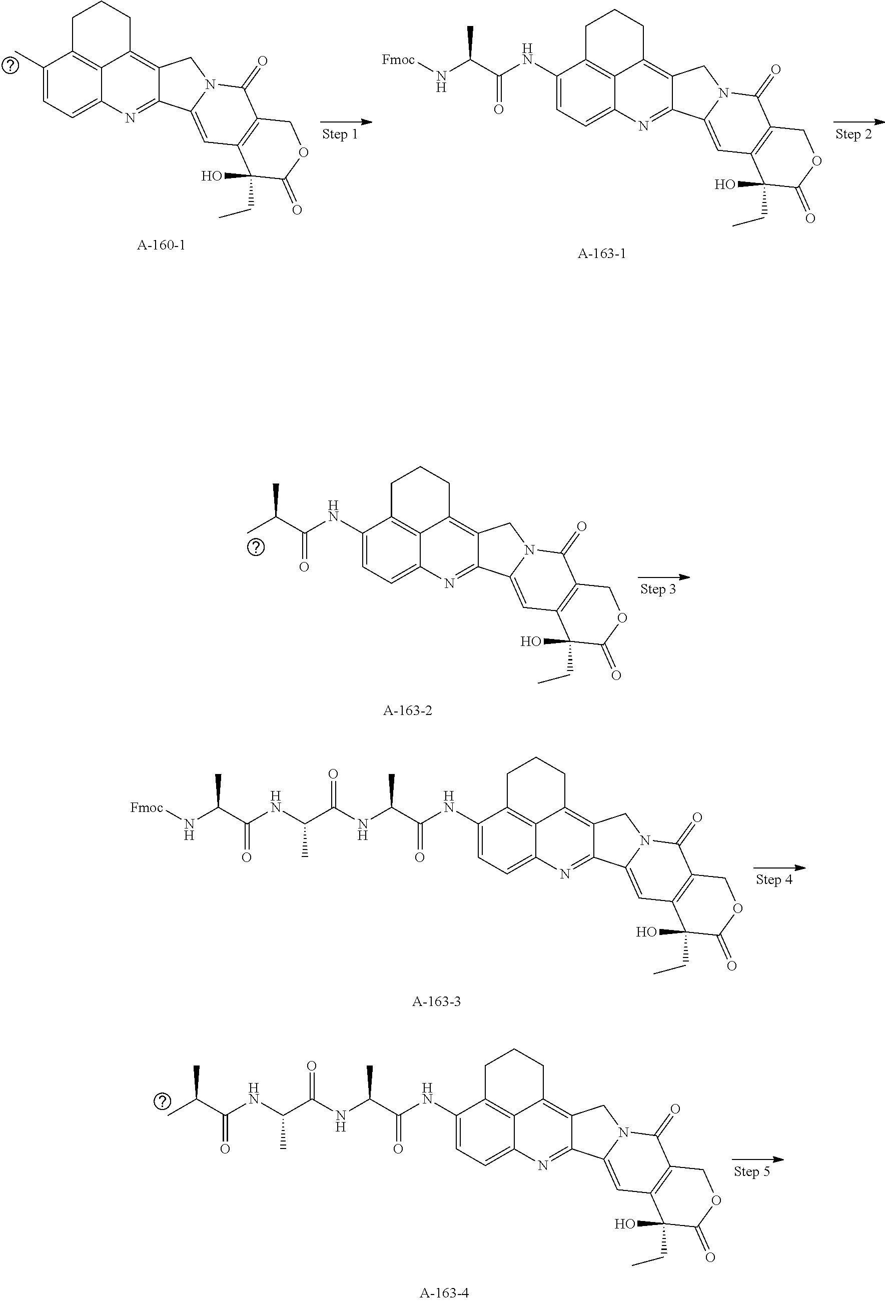

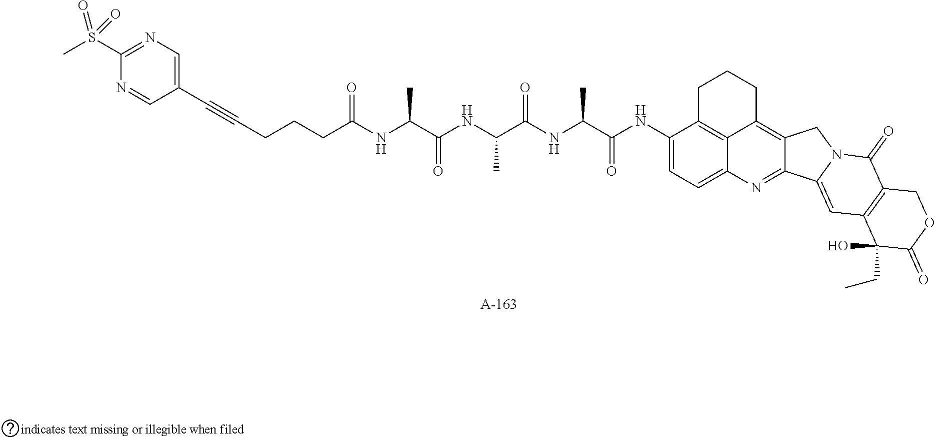 [SMILES: CC[C@@]1(O)C(=O)OCc2c1cc1n(c2=O)Cc2c-1nc1ccc(NC(=O)[C@H](C)NC(=O)[C@H](C)NC(=O)[C@H](C)NC(=O)CCCC#Cc3cnc(S(C)(=O)=O)nc3)c3c1c2CCC3 ; "A-163"]

### Step 1 Preparation of (9H-fluoren-9-yl)methyl((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)carbamate (A-163-1)

Fmoc-L-alanine (154.34 mg, 495.75 μmol), (S)-4-amino-9-ethyl-9-hydroxy-1,2,3,9,12,15-hexahydro-10H,13H-benzo[de]pyrano[3′,4′:6,7]indolizino[1,2-b]quinolin-10,13-dione (50 mg, 0.12 mmol) and HATU (188.38 mg, 495.75 μmol) were dissolved in dry DMF (2 mL), added dropwise with DIPEA (96.11 mg, 743.62 μmol), and reacted under stirring and nitrogen protection for 12 hours. The reaction solution was added dropwise with 1N hydrochloric acid to neutralize the reaction, and solid precipitated. After filtration, the solid washed with water and dried to obtain a crude product (162 mg, 232.51 μmol), which was directly used in the next step.

Its structural characterization data are as follows:

MS m/z (ESI): 696.7[M+H]⁺

### Step 2: Preparation of (S)-2-amino-N—((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)propanamide (A-163-2)

(9H-Fluoren-9-yl)methyl ((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)carbamate (162 mg, 232.51 μmol) was dissolved in DMF (1.5 mL), added dropwise with diethylamine (0.5 mL) under stirring, and reacted continuously for 0.5 hours. The reaction solution was subjected to flash column chromatography (C18, water/acetonitrile=2/1), and then freeze-dried to obtain the title compound (55 mg, 0.11 mmol).

Its structural characterization data are as follows:

MS m/z (ESI): 475.4[M+H]⁺

### Step 3: Preparation of (9H-fluoren-9-yl)methyl ((S)-1-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-1-oxopropan-2-yl)amino)-1-oxopropan-2-yl)carbamate (A-163-3)

(((9H-Fluoren-9-yl)methoxy)carbonyl)-L-alanyl-L-alanine (48.49 mg, 126.79 μmol), (S)-2-amino-N—((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)propanamide (55 mg, 105.66 μmol) and HATU (80.30 mg, 211.32 μmol) were dissolved in dry DMF (2 mL), added dropwise with DIPEA (13.66 mg, 105.66 μmol), and reacted under stirring for 1 hour. The reaction solution was added with water to precipitate solid, which was filtered, washed with water and dried to obtain the crude product (88 mg, 104.90 μmol).

Its structural characterization data are as follows:

MS m/z (ESI): 839.6[M+H]⁺

### Step 4: Preparation of (S)-2-amino-N—((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-1-oxopropan-2-yl)propanamide (A-163-4)

(9H-Fluoren-9-yl)methyl ((S)-1-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-1-oxopropan-2-yl)amino)-1-oxopropan-2-yl)carbamate (88 mg, 104.90 μmol) was dissolved in DMF (2 mL), added dropwise with diethylamine (0.5 mL) under stirring, and reacted continuously for 0.5 hours. The reaction solution was evaporated under reduced pressure to remove diethylamine, added dropwise with 1N hydrochloric acid to acidify, then subjected to flash column chromatography (C18, water/acetonitrile=3/1), and freeze-dried to obtain the title compound (32 mg, 48.29 μmol).

Its structural characterization data are as follows:

MS m/z (ESI): 617.4[M+H]⁺

### Step 5: Preparation of N—((S)-1-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-1-oxopropan-2-yl)amino)-1-oxopropan-2-yl)-6-(2-(methylsulfonyl)pyrimidin-5-yl)hex-5-ynamide (A-163)

2,5-Dioxopyrrolidin-1-yl 6-(2-(methylsulfonyl)pyrimidin-5-yl)hex-5-ynoate (13.23 mg, 36.22 μmol), (S)-2-amino-N—((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-1-oxopropan-2-yl)propanamide (20 mg, 30.18 μmol) and DIPEA (7.80 mg, 60.36 μmol) were dissolved in DMF (1 mL), and reacted under stirring for 1 hour. The reaction solution was directly purified by preparative high performance liquid chromatography and freeze-dried to obtain the title compound (17.70 mg, 19.40 μmol).

Its structural characterization data are as follows:

MS m/z (ESI): 867.4[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 20.00 | 90 | 10 | 28 |

### Example 47: Preparation of N—((S)-1-(((S)-6-amino-1-((2-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)amino)-1-oxohexan-2-yl)amino)-3-methyl-1-oxobutan-2-yl)-6-(2-(methylsulfonyl)pyrimidin-5-yl)hex-5-ynamide (A-164)

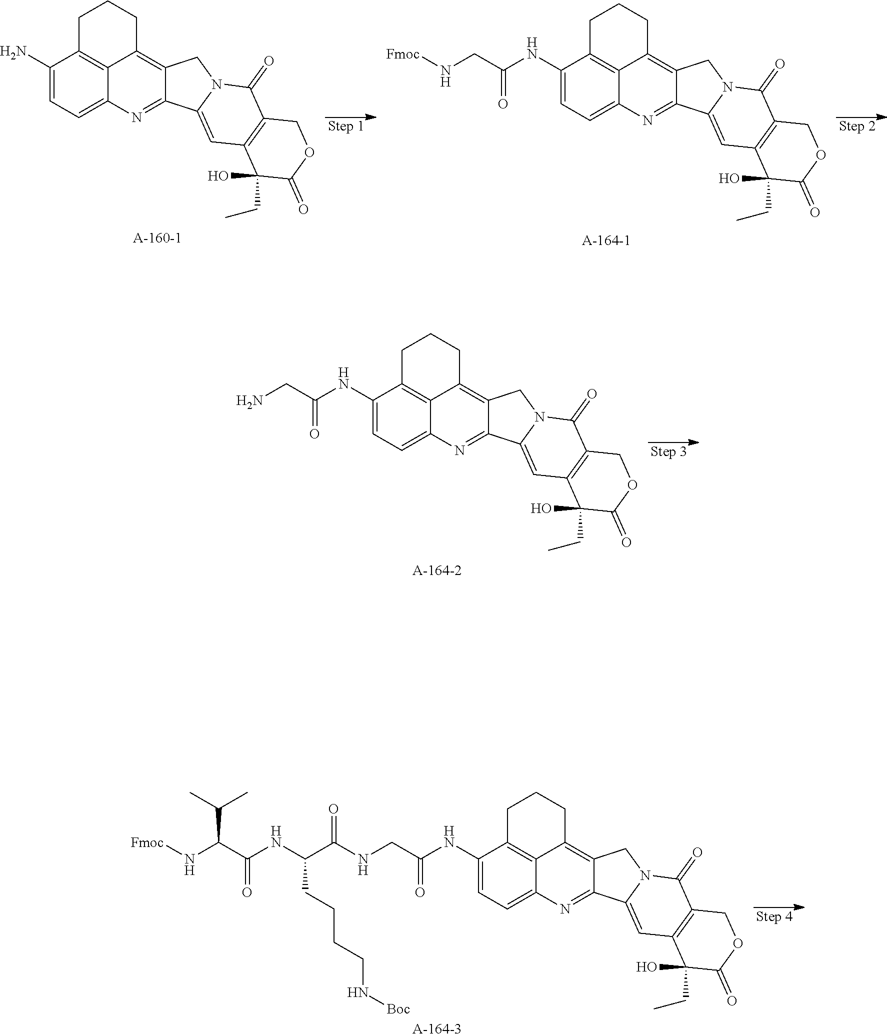

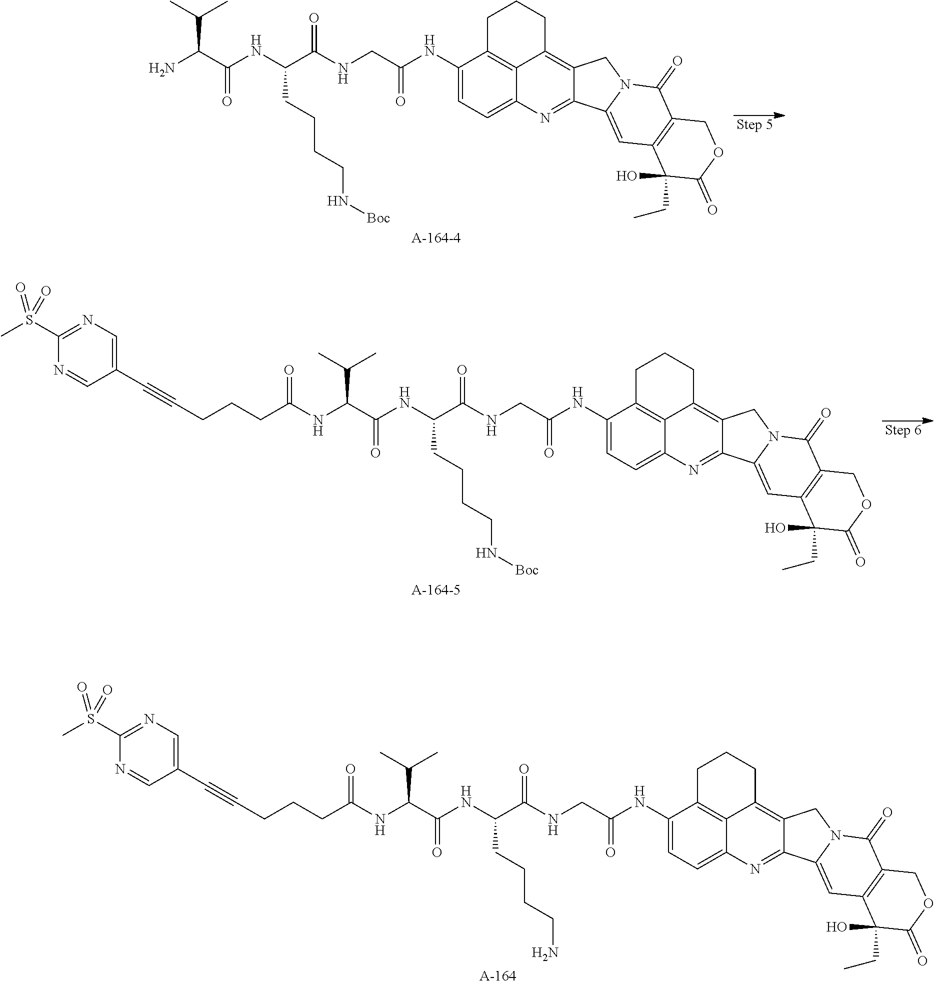

### Step 1: Preparation of (9H-fluoren-9-yl)methyl (S)-(2-((9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)carbamate (A-164-1)

(((9H-Fluoren-9-yl)methoxy)carbonyl)glycine (73.69 mg, 247.87 μmol), (S)-4-amino-9-ethyl-9-hydroxy-1,2,3,9,12,15-hexahydro-10H,13H-benzo[de]pyrano[3′,4′:6,7]indolizino[1,2-b]quinolin-10,13-dione (50 mg, 123.94 μmol) and HATU (94.19 mg, 247.87 μmol) were dissolved in dry DMF (1 mL), added dropwise with DIPEA (48.05 mg, 371.81 μmol), and reacted under stirring and nitrogen protection for 12 hours. The reaction solution was added dropwise with 0.5N hydrochloric acid aqueous solution to neutralize the reaction, and solid precipitated and filtered. The solid was washed with water and then dried to obtain a crude product (80 mg, 117.18 μmol), which was directly used in the next step.

Its structural characterization data are as follows:

MS m/z (ESI): 683.3[M+H]⁺

### Step 2: Preparation of (S)-2-amino-N-(9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)acetamide (A-164-2)

(9H-Fluoren-9-yl)methyl (S)-(2-((9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)carbamate (80 mg, 117.18 μmol) was dissolved in DMF (1 mL), added dropwise with diethylamine (0.1 mL), and reacted under stirring for 0.5 hours. The reaction solution was subjected to flash column chromatography (C18, water/acetonitrile=2/1), and then freeze-dried to obtain the title compound (42 mg, 82.92 μmol).

Its structural characterization data are as follows:

MS m/z (ESI): 460.5[M+H]⁺

### Step 3: Preparation of tert-butyl ((S)-5-((S)-2-((((9H-fluoren-9-yl)methoxy)carbonyl) amino)-3-methylbutanamido)-6-((2-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)amino)-6-oxohexyl)carbamate (A-164-3)

N²-((((9H-Fluoren-9-yl)methoxy)carbonyl)-L-valyl)-N⁶-(tert-butoxycarbonyl)-L-lysine (56.49 mg, 99.51 μmol) and (S)-2-amino-N-(9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)acetamide (42 mg, 82.92 μmol) were dissolved in DMF (2 mL), added with HATU (47.26 mg, 124.38 μmol), then added dropwise with DIPEA (10.72 mg, 82.92 μmol), and reacted under stirring for 1 hour. The reaction solution was poured into water to precipitate solid. The solid was filtered, washed with water, and dried to obtain a crude product (80 mg, 79.20 mol), which was directly used in the next step.

Its structural characterization data are as follows:

MS m/z (ESI): 1010.3[M+H]⁺

### Step 4: Preparation of tert-butyl ((S)-5-((S)-2-amino-3-methylbutanamido)-6-((2-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)amino)-6-oxohexyl)carbamate (A-164-4)

Tert-butyl ((S)-5-((S)-2-((((9H-fluoren-9-yl)methoxy)carbonyl)amino)-3-methyl-butanamido)-6-((2-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)amino)-6-oxohexyl)carbamate (80 mg, 79.20 μmol) was dissolved in DMF (1 mL), added dropwise with diethylamine (0.5 mL), and reacted under stirring for 0.5 hours. The reaction solution was added dropwise with 1N hydrochloric acid aqueous solution for neutralization, then purified by preparative high performance liquid chromatography and freeze-dried to obtain the title compound (15 mg, 17.99 μmol).

Its structural characterization data are as follows:

MS m/z (ESI): 788.5[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 5 | 95 | 30 |
| 2.00 | 5 | 95 | 30 |
| 20.00 | 90 | 10 | 30 |

### Step 5: Preparation of tert-butyl ((S)-6-((2-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)amino)-5-((S)-3-methyl-2-(6-(2-(methylsulfonyl)pyrimidin-5-yl)hex-5-ynamido)butanamido)-6-oxohexyl)carbamate (A-164-5)

2,5-Dioxopyrrolidin-1-yl 6-(2-(methylsulfonyl)pyrimidin-5-yl)hex-5-ynoate (7.89 mg, 21.58 μmol) and tert-butyl ((S)-5-((S)-2-amino-3-methylbutanamido)-6-((2-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)amino)-6-oxohexyl)carbamate (15 mg, 17.99 μmol) were dissolved in DMF (1 mL), added dropwise with DIPEA (2.32 mg, 17.99 μmol) under stirring, and reacted continuously for 1 hour. The reaction solution was directly purified by preparative high performance liquid chromatography and freeze-dried to obtain the title compound (5 mg, 4.82 μmol).

Its structural characterization data are as follows:

MS m/z (ESI): 1039.5[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 30 |
| 4.00 | 30 | 70 | 30 |
| 24.00 | 90 | 10 | 30 |

### Step 6: Preparation of N—((S)-1-(((S)-6-amino-1-((2-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)amino)-1-oxohex-2-yl)amino)-3-methyl-1-oxobutan-2-yl)-6-(2-(methylsulfonyl)pyrimidin-5-yl)hex-5-ynamide (A-164)

Tert-butyl ((S)-6-((2-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-2-oxoethyl)amino)-5-((S)-3-methyl-2-(6-(2-(methylsulfonyl)pyrimidin-5-yl)hex-5-ynamido)butanamido)-6-oxohexyl)carbamate (5 mg, 4.82 μmol) was dissolved in dry dichloromethane (0.5 mL), added dropwise with TFA (0.1 mL) under stirring, and reacted continuously for 1 hour. The reaction solution was added dropwise with sodium bicarbonate aqueous solution for neutralization, and extracted with dichloromethane. The organic phase was concentrated under reduced pressure, purified by preparative high performance liquid chromatography, and freeze-dried to obtain the title compound (1.52 mg, 1.53 μmol).

Its structural characterization data are as follows:

MS m/z (ESI): 939.5[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 3 | 97 | 30 |
| 2.00 | 3 | 97 | 30 |
| 20.00 | 90 | 10 | 30 |

### Example 48: Preparation of N-((35S,38S)-39-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-35-isopropyl-3,6,9,12,15,18,21,24,27,38-decamethyl-2,5,8,11,14,17,20,23,26,29,33,36,39-tridecaoxo-3,6,9,12,15,18,21,24,27,30,34,37-dodecazanonatriacontanyl)-N-methyl-6-(2-(methylsulfonyl)pyrimidin-5-yl)hex-5-ynamide (A-201)

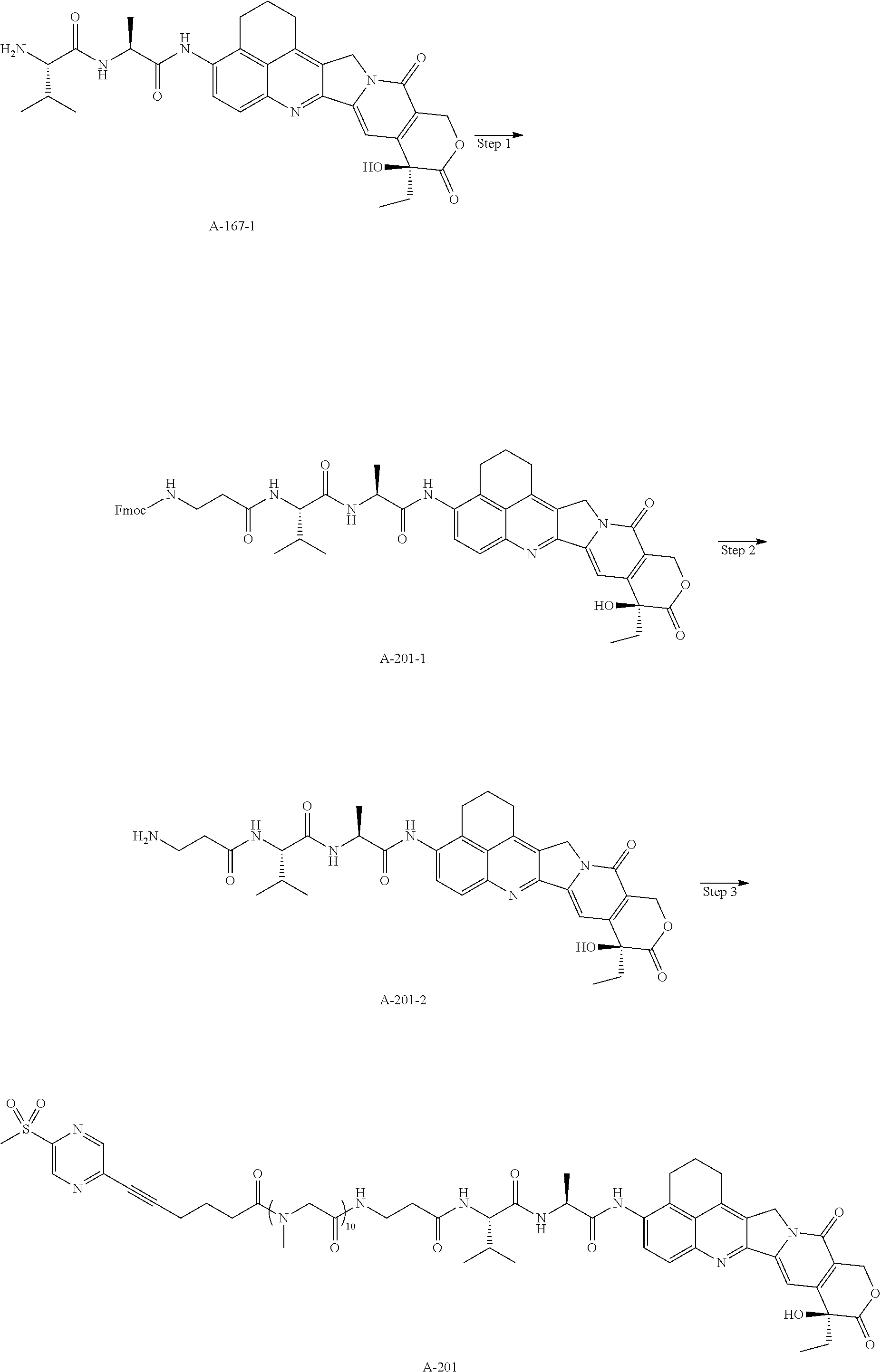

### Step 1: Preparation of (9H-fluoren-9-yl)methyl (3-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutyl-2-yl)amino)-3-oxopropyl)carbamate (A-201-1)

(S)-2-Amino-N—((S)-1-(((S)-9-ethyl-9-hydroxy-10,13-dioxo-2,3,9,10,13,15-hexahydro)-1H,12H-benzo[de]pyrano[3′,4′:6,7]indolizino[1,2-b]quinolin-4-yl)amino)-1-oxopropan-2-yl)-3-methylbutanamide (20.0 mg, 0.035 mmol) and 3-((((9H-fluoren-9-yl)methoxy)carbonyl)amino) propionic acid (16.3 mg, 0.05 mmol) were dissolved in DMF (2 mL), added with 2-(7-azobenzotriazole)-N,N,N′,N′-tetramethyluronium hexafluorophosphate (19.9 mg, 0.05 mmol) and DIPEA (23.9 mg, 0.06 mmol), and stirred at room temperature for 1 hour. After the reaction was completed, the reaction solution was added with 10 mL of water to precipitate solid. The resultant filter cake after filtration was freeze-dried to remove moisture to obtain the title compound (21.3 mg, 0.025 mmol), which was used directly in the next step without purification.

Its structural characterization data are as follows:

MS m/z (ESI): 867.2 [M+H]⁺

### Step 2: Preparation of (S)-2-(3-aminopropionamido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (A-201-2)

(9H-Fluoren-9-yl)methyl (3-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutyl-2-yl)amino)-3-oxopropyl)carbamate (21.3 mg, 0.025 mmol) was dissolved in DMF (2 mL), added with diethylamine (18.1 mg, 0.25 mmol), and stirred at room temperature for 1 hour. After the reaction was completed, the reaction solution was dried on a freeze dryer to remove the solvent, then added with 10 mL of a mixed solvent (EA/PE=1/3) for recrystallization for 2 hours, and filtered to obtain the title compound (13.9 mg, 0.023 mmol).

Its structural characterization data are as follows:

MS m/z (ESI): 617.2 [M+H]⁺

### Step 3: Preparation of N-((35S,38S)-39-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-35-isopropyl-3,6,9,12,15,18,21,24,27,38-decamethyl-2,5,8,11,14,17,20,23,26,29,33,36,39-tridecaoxo-3,6,9,12,15,18,21,24,27,30,34,37-dodecazanonatriacontanyl)-N-methyl-6-(2-(methylsulfonyl)pyrimidin-5-yl)hex-5-ynamide (A-201)

(S)-2-(3-Aminopropionamido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (13.9 mg, 0.023 mmol) and INT-1 (33.7 mg, 0.035 mmol) were dissolved in DMF (2 mL), added with 2-(7-azobenzotriazole)-N,N,N′,N′-tetramethyluronium hexafluorophosphate (17.5 mg, 0.046 mmol) and DIPEA (14.9 mg, 0.115 mmol), and stirred at room temperature for 1 hour. After the reaction was completed, the reaction solution was directly purified by preparative high performance liquid chromatography and freeze-dried to obtain the title compound (5.45 mg, 3.45 μmol).

Its structural characterization data are as follows:

MS m/z (ESI): 1577.3 [M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 10 | 90 | 32 |
| 4.00 | 10 | 90 | 32 |
| 24.00 | 70 | 30 | 32 |

### Example 49: Preparation of (R)-4-amino-9-ethyl-10,13-dioxo-2,3,9,10,13,15-hexahydro-1H,12H-benzo[de]pyrano[3′,4′:6,7]indolizino[1,2-b]quinolin-9-yl (4-((S)-2-(4-aminobutyl)-35-(4-((6-(2-(methylsulfonyl)pyrimidin-5-)yl)hex-5-ynamido)methyl)-1H-1,2,3-triazol-1-yl)-4,8-dioxo-6,12,15,18,21,24,27,30,33-nonaoxa-3,9-diazapentatriacontanamido)benzyl)carbonate (A-154)

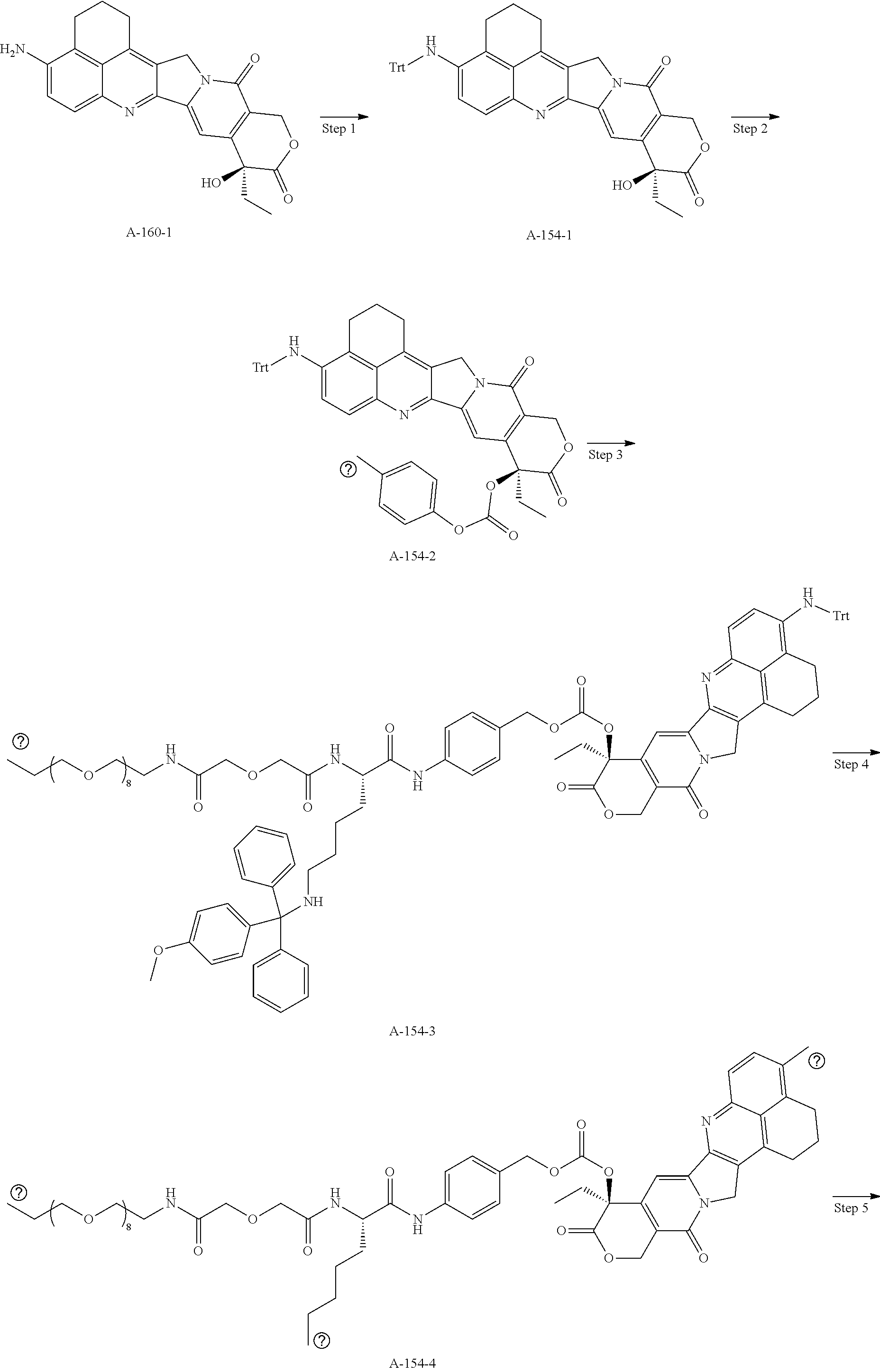

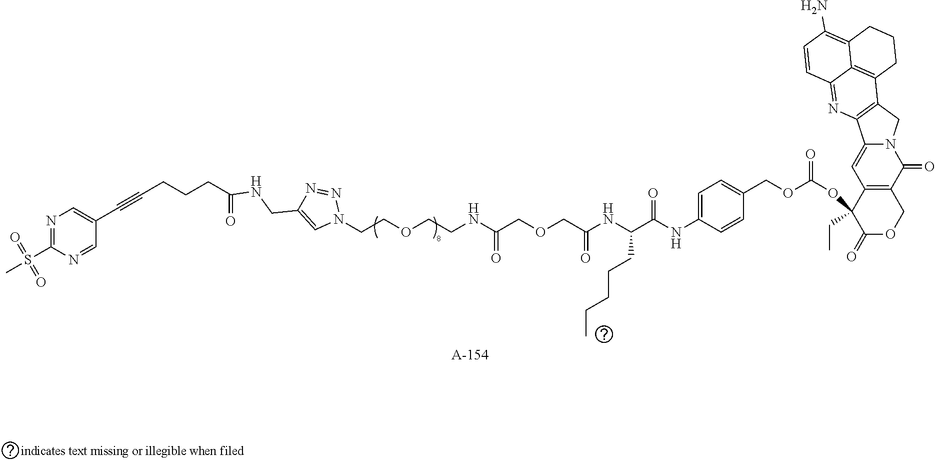

### Step 1: Preparation of (S)-9-ethyl-9-hydroxy-4-(tritylamino)-1,2,3,9,12,15-hexahydro-10H,13H-benzo[de]pyrano[3′,4′:6, 7]indolizino[1,2-b]quinolin-10,13-dione (A-154-1)

Triphenylmethane chloride (207.30 mg, 0.74 mmol), (S)-4-amino-9-ethyl-9-hydroxy-1,2,3,9,12,15-hexahydro-10H,13H-benzo[de]pyrano[3′,4′:6,7]indolizino[1,2-b]quinolin-10,13-dione (200 mg, 0.50 mmol) and DMAP (6.06 mg, 49.57 μmol) were dissolved in dry DMF (4 mL), added dropwise with triethylamine (100.33 mg, 1.0 mmol), heated to 50° C. and reacted for 12 hours. The reaction mixture was cooled to room temperature and poured into water to precipitate solid. The solid was filtered, washed with water and dried to obtain a crude product (310 mg, 0.48 mmol), which was used directly in the next step without purification.

### Step 2: Preparation of (S)-9-ethyl-10,13-dioxo-4-(tritylamino)-2,3,9,10,13,15-hexahydro-1H,12H-benzo[de]pyrano[3′,4′:6,7]indolizino[1,2-b]quinolin-9-yl (4-nitrophenyl)carbonate (A-154-2)

(S)-9-Ethyl-9-hydroxy-4-(tritylamino)-1,2,3,9,12,15-hexahydro-10H,13H-benzo[de]pyrano[3′,4′:6,7]indolizino[1,2-b]quinolin-10,13-dione (310 mg, 0.48 mmol) and DMAP (175.95 mg, 1.44 mmol) were dissolved in dry dichloromethane. (10 mL), added with p-nitrophenol chloroformate (290.29 mg, 1.44 mmol), and reacted continuously under stirring for 3 hours. The reaction solution was directly purified by flash silica gel column chromatography (0-70% ethyl acetate/petroleum ether) to obtain the title compound (327 mg, 0.40 mmol).

Its structural characterization data are as follows:

MS m/z (ESI): 810.8[M+H]⁺

### Step 3: Preparation of 4-((S)-35-azido-2-(4-(((4-methoxyphenyl)diphenylmethyl)amino) butyl)-4,8-dioxo-6,12,15,18,21,24,27,30,33-nonaoxa-3,9-diazapentatriacontanamido)benzyl ((R)-9-ethyl-10,13-dioxo-4-(tritylamino)-2,3,9,10,13,15-hexahydro-1H,12H-benzo[de]pyrano[3′,4′:6,7]indolizino[1,2-b]quinolin-9-yl)carbonate (A-154-3)

(S)-2-(32-Azido-5-oxo-3,9,12,15,18,21,24,27,30-nonaoxa-6-azadotriacontanamido)-N-(4-(hydroxymethyl)phenyl)-6-(((4-methoxyphenyl)diphenylmethyl)amino)hexanamide (427.57 mg, 0.40 mmol), (S)-9-ethyl-10,13-dioxo-4-(tritylamino)-2,3,9,10,13,15-hexahydro-1H,12H-benzo[de]pyrano[3′,4′:6,7]indolizino[1,2-b]quinolin-9-yl(4-nitrophenyl)carbonate (327 mg, 0.40 mmol) and DMAP (147.81 mg, 1.21 mmol) were dissolved in dry DMF (5 mL), and reacted under stirring for 12 hours. The reaction solution was added with ethyl acetate for dilution, and washed three times with saturated brine. The organic phase was dried, concentrated, and purified by flash silica gel column chromatography (0-5% methanol/dichloromethane) to obtain the target compound (270 mg, 0.16 mmol).

Its structural characterization data are as follows:

MS m/z (ESI): 1732.2[M+H]⁺

### Step 4: Preparation of (R)-4-amino-9-ethyl-10,13-dioxo-2,3,9,10,13,15-hexahydro-1H,12H-benzo[de]pyrano[3′,4′:6,7]indolizino[1,2-b]quinolin-9-yl (4-((S)-2-(4-aminobutyl)-35-azido-4,8-dioxo-6,12,15,18,21,24,27,30,33-nonaoxa-3,9-diazapentatriacontanamido)benzyl)carbonate (A-154-4)

4-((S)-35-Azido-2-(4-(((4-methoxyphenyl)diphenylmethyl)amino)butyl)-4,8-dioxo-6,12,15,18,21,24,27,30,33-nonaoxa-3,9-diazapentatriacontanamido)benzyl ((R)-9-ethyl-10,13-dioxo-4-(tritylamino)-2,3,9,10,13,15-hexahydro-1H,12H-benzo[de]pyrano[3′,4′:6,7]indolizino[1,2-b]quinolin-9-yl)carbonate (270 mg, 0.16 mmol) was dissolved in acetonitrile (5 mL), added dropwise with formic acid (0.5 mL) under stirring, and reacted for 1 hour. The reaction solution was purified directly by preparative high performance liquid chromatography and freeze-dried to obtain the title compound (166 mg, 0.13 mmol).

Its structural characterization data are as follows:

MS m/z (ESI): 1217.3[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 10 | 90 | 30 |
| 2.00 | 10 | 90 | 30 |
| 20.00 | 90 | 10 | 30 |

### Step 5: Preparation of (R)-4-amino-9-ethyl-10,13-dioxo-2,3,9,10,13,15-hexahydro-1H,12H-benzo[de]pyrano[3′,4′:6,7]indolizino[1,2-b]quinolin-9-yl (4-((S)-2-(4-aminobutyl)-35-(4-((6-(2-(methylsulfonyl)pyrimidin-5-yl)hex-5-ynamido)methyl)-1H-1,2,3-triazol-1-yl)-4,8-dioxo-6,12,15,18,21,24,27,30,33-nonaoxa-3,9-diazapentatriacontanamido)benzyl)carbonate (A-154)

6-(2-Methylsulfonylpyrimidin-5-yl)-N-propyl-2-ynylhex-5-amide (7.53 mg, 24.64 μmol), (R)-4-amino-9-ethyl-10,13-dioxo-2,3,9,10,13,15-hexahydro-1H,12H-benzo[de]pyrano[3′,4′:6,7]indolizino[1,2-b]quinolin-9-yl (4-((S)-2-(4-aminobutyl)-35-azido-4,8-dioxo-6,12,15,18,21,24,27,30,33-nonaoxa-3,9-diazapentatriacontanamido)benzyl)carbonate (30 mg, 24.64 μmol) and CuBr (3.54 mg, 24.64 μmol) were dissolved in DMSO (1 mL) and water (0.1 mL), and stirred for 1 hour. The reaction solution was filtered, purified by preparative high performance liquid chromatography and freeze-dried to obtain the target compound (23.03 mg, 13.21 μmol).

Its structural characterization data are as follows:

MS m/z (ESI): 1522.3[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 20 | 80 | 30 |
| 4.00 | 20 | 80 | 30 |
| 24.00 | 90 | 10 | 30 |

### Example 50: Preparation of 4-((S)-2-(4-aminobutyl)-35-(4-((6-(2-(methylsulfonyl) pyrimidin-5-yl)hex-5-ynamido)methyl)-1H-1,2,3-triazol-1-yl)-4,8-dioxo-6,12,15,18,21,24,27,30,33-nonaoxa-3,9-diazapentatriacontanamido)benzyl ((R)-9-ethyl-4-hydroxy-10,13-dioxo-2,3,9,10,13,15-hexahydro-1H,12H-benzo[de]pyrano[3′,4′:6,7]indolizino[1,2-b]quinolin-9-yl)carbonate (A-155)

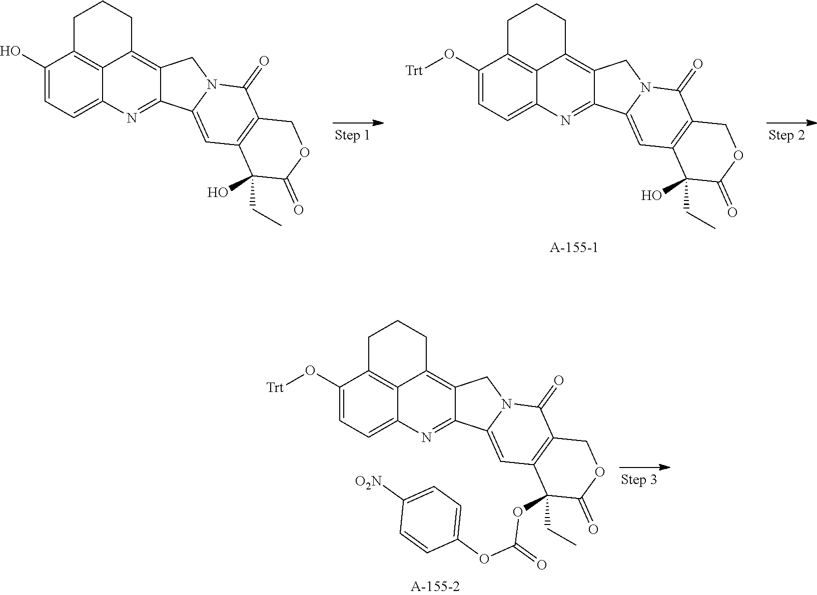

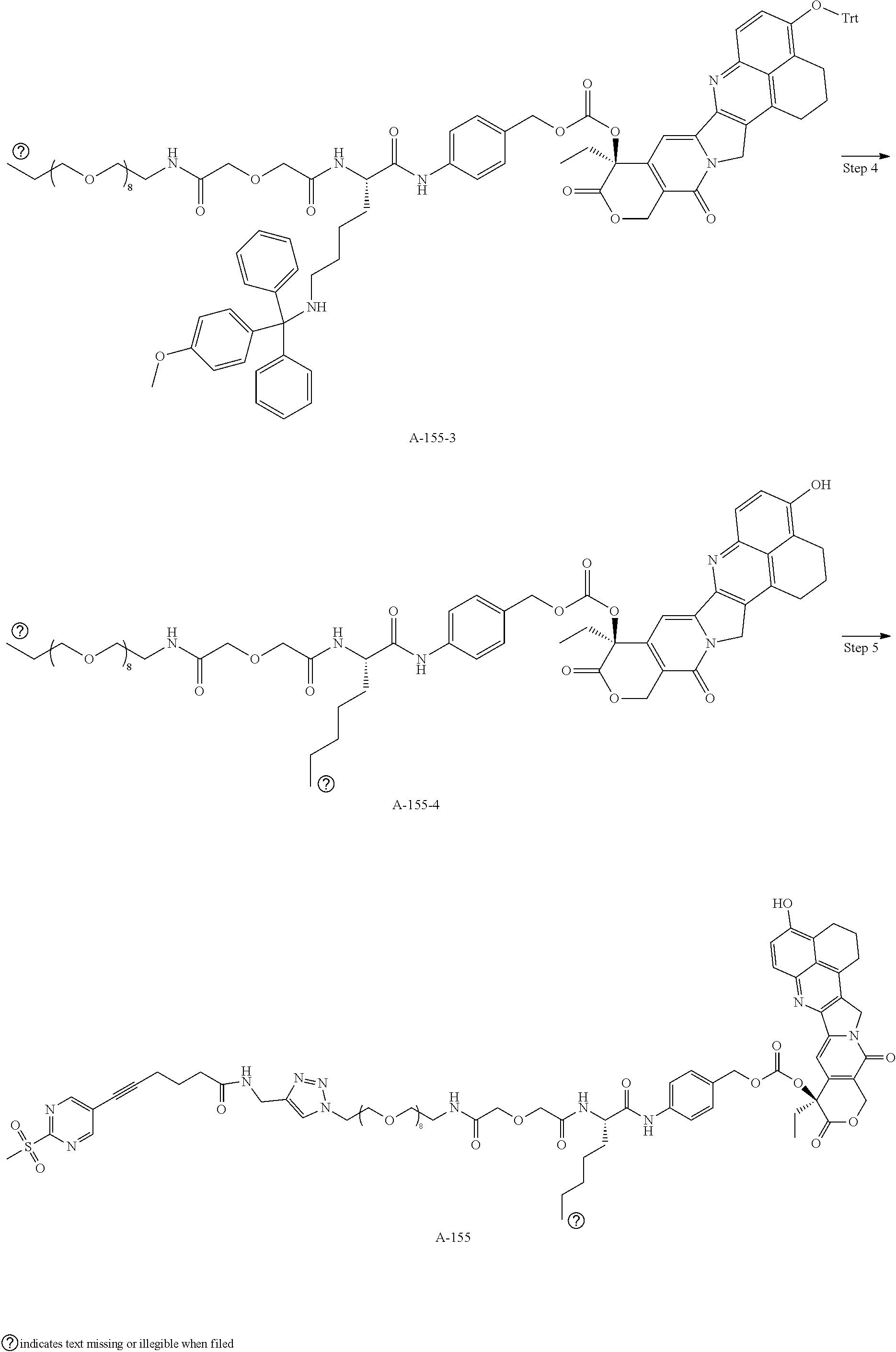

### Step 1: Preparation of(S)-9-ethyl-9-hydroxy-4-(trityloxy)-1,2,3,9,12,15-hexahydro-10H,13H-benzo[de]pyrano[3′,4′: 6,7]indolizino[1,2-b]quinolin-10,13-dione (A-155-1)

Triphenylmethane chloride (2.07 g, 7.42 mmol), (S)-4-hydroxy-9-ethyl-9-hydroxy-1,2,3,9,12,15-hexahydro-10H,13H-benzo[de]pyrano[3′,4′: 6,7]indolizino[1,2-b]quinolin-10,13-dione (1 g, 2.47 mmol) and DMAP (60.42 mg, 494.54 μmol) was dissolved in dry DMF (20 mL), added dropwise with triethylamine (750.64 mg, 7.42 mmol), then heated to 50° C. and reacted for 12 hours. The reaction solution was cooled to room temperature, poured into water to precipitate solid, and filtered. The solid was washed with water and dried to obtain a crude product. The crude product was purified by flash silica gel chromatography (30-80% ethyl acetate/petroleum ether) to obtain the target product (1.37 g, 2.12 mmol), which was used directly in the next step without purification.

### Step 2: Preparation of (S)-9-ethyl-10,13-dioxo-4-(trityloxy)-2,3,9,10,13,15-hexahydro-1H,12H-benzo[de]pyrano[3′,4′:6,7]indolizino[1,2-b]quinolin-9-yl(4-nitrophenyl)carbonate (A-155-2)

(S)-9-Ethyl-9-hydroxy-4-(trityloxy)-1,2,3,9,12,15-hexahydro-10H,13H-benzo[de]pyrano[3′,4′:6,7]indolizino[1,2-b]quinolin-10,13-dione (100 mg, 0.15 mmol) and DMAP (56.67 mg, 0.45 mmol) were dissolved in dry dichloromethane (2 mL), added with p-nitrophenol chloroformate (93.50 mg, 0.45 mmol), and reacted continuously under stirring for 3 hours. The reaction solution was directly purified by flash silica gel chromatography (0-70% ethyl acetate/petroleum ether) to obtain the target compound (68 mg, 0.08 mmol).

Its structural characterization data are as follows:

MS m/z (ESI): 811.8[M+H]⁺

### Step 3: Preparation of 4-((S)-35-azido-2-(4-(((4-methoxyphenyl)diphenylmethyl)amino) butyl)-4,8-dioxo-6,12,15,18,21,24,27,30,33-nonaoxa-3,9-diazapentatriacontanamido)benzyl ((R)-9-ethyl-10,13-dioxo-4-(trityloxy)-2,3,9,10,13,15-hexahydro-1H,12H-benzo[de]pyrano[3′,4′:6,7]indolizino[1,2-b]quinolin-9-yl)carbonate (A-155-3)

(S)-2-(32-Azido-5-oxo-3,9,12,15,18,21,24,27,30-nonaoxa-6-azadotriacontanamido)-N-(4-(hydroxymethyl)phenyl)-6-(((4-methoxyphenyl)diphenylmethyl)amino)hexanamide (88.81 mg, 83.76 μmol), (S)-9-ethyl-10,13-dioxo-4-(trityloxy)-2,3,9,10,13,15-hexahydro-1H,12H-benzo[de]pyrano[3′,4′:6,7]indolizino[1,2-b]quinolin-9-yl (4-nitrophenyl)carbonate (68 mg, 83.76 μmol) and DMAP (30.70 mg, 251.28 μmol) were dissolved in dry DMF (1 mL), and reacted under stirring for 12 hours. The reaction solution was added with ethyl acetate for dilution, and washed with saturated brine three times. The organic phase was dried, concentrated, and purified by silica gel column chromatography (0-5% methanol/dichloromethane) to obtain the target compound (42 mg, 24.24 μmol).

Its structural characterization data are as follows:

MS m/z (ESI): 1733.3[M+H]⁺

### Step 4: Preparation of 4-((S)-2-4(-aminobutyl)-35-azido-4,8-dioxo-6,12,15,18,21,24,27,30,33-nonaoxa-3,9-diazapentatriacontanamido)benzyl((R)-9-ethyl-4-hydroxy-10,13-diazo-2,3.9,10,13,15-hexahydro-1H,12H-benzo[de]pyrano[3′,4′:6,7]indolizino[1,2-b]quinoline-9-yl)carbonate (A155-4)

4-((S)-35-Azido-2-(4-(((4-methoxyphenyl)diphenylmethyl)amino)butyl)-4,8-dioxo-6,12,15,18,21,24,27,30,33-nonaoxa-3,9-diazapentatriacontanamido)benzyl ((R)-9-ethyl-10,13-dioxo-4-(trityloxy)-2,3,9,10,13,15-hexahydro-1H,12H-benzo[de]pyrano[3′,4′:6,7]indolizino[1,2-b]quinolin-9-yl)carbonate (42 mg, 24.24 μmol) was dissolved in acetonitrile (2 mL), added dropwise with formic acid (0.2 mL) under stirring, and reacted for 1 hour. The reaction solution was purified directly by preparative high performance liquid chromatography and freeze-dried to obtain the target compound (22 mg, 17.40 μmol).

Its structural characterization data are as follows:

MS m/z (ESI): 1219.3[M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 5 | 95 | 30 |
| 2.00 | 5 | 95 | 30 |
| 20.00 | 90 | 10 | 30 |

### Step 5: Preparation of 4-((S)-2-(4-aminobutyl)-35-(4-((6-(2-(methylsulfonyl)pyrimidin-5-yl) hex-5-ynamide)methyl)-1H-1,2,3-triazol-1-yl)-4,8-dioxo-6,12,15,18,21,24,27,30,33-nonaoxa-3,9-diazapentatriacontanamido)benzyl ((R)-9-ethyl-4-hydroxyl-10,13-dioxo-2,3,9,10,13,15-hexahydro-1H,12H-benzo[de]pyrano[3′,4′:6,7]indolizino[1,2-b]quinolin-9-yl)carbonate (A-155)

6-(2-Methylsulfonylpyrimidin-5-yl)-N-propyl-2-ynylhexan-5-amide (5.31 mg, 17.40 mol), 4-((S)-2-(4-aminobutyl)-35-azido-4,8-dioxo-6,12,15,18,21,24,27,30,33-nonaoxa-3,9-diazapentatriacontanamido)benzyl((R)-9-ethyl-4-hydroxy-10,13-dioxo-2,3,9,10,13,15-hexahydro-1H,12H-benzo[de]pyrano[3′,4′:6,7]indolizino[1,2-b]quinoline-9-yl]carbonate (22 mg, 17.40 μmol) and CuBr (2.50 mg, 17.40 μmol) were dissolved in DMSO (1 mL) and water (0.1 mL), and reacted under stirring for 1 hour. The reaction solution was filtered, purified by preparative high performance liquid chromatography and freeze-dried to obtain the target compound (6.08 mg, 3.80 μmol).

Its structural characterization data are as follows:

MS m/z (ESI): 1523.8 [M+H]⁺

Its preparation method was as follows:

Chromatographic column: Waters Xbridge Prep C18 OBD
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 20 | 80 | 30 |
| 4.00 | 20 | 80 | 30 |
| 24.00 | 90 | 10 | 30 |

### Example 51: Preparation of 2,2′,2″-(10-(2-(((S)-6-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutan-2-yl)amino)-5-(6-(2-(methylsulfonyl)pyrimidin-5-yl)hex-5-ynamido)-6-oxohexyl)amino)-2-oxoethyl)-1,4,7,10-tetraazacyclododecan-1,4,7-triyl)triacetic acid (B-13)

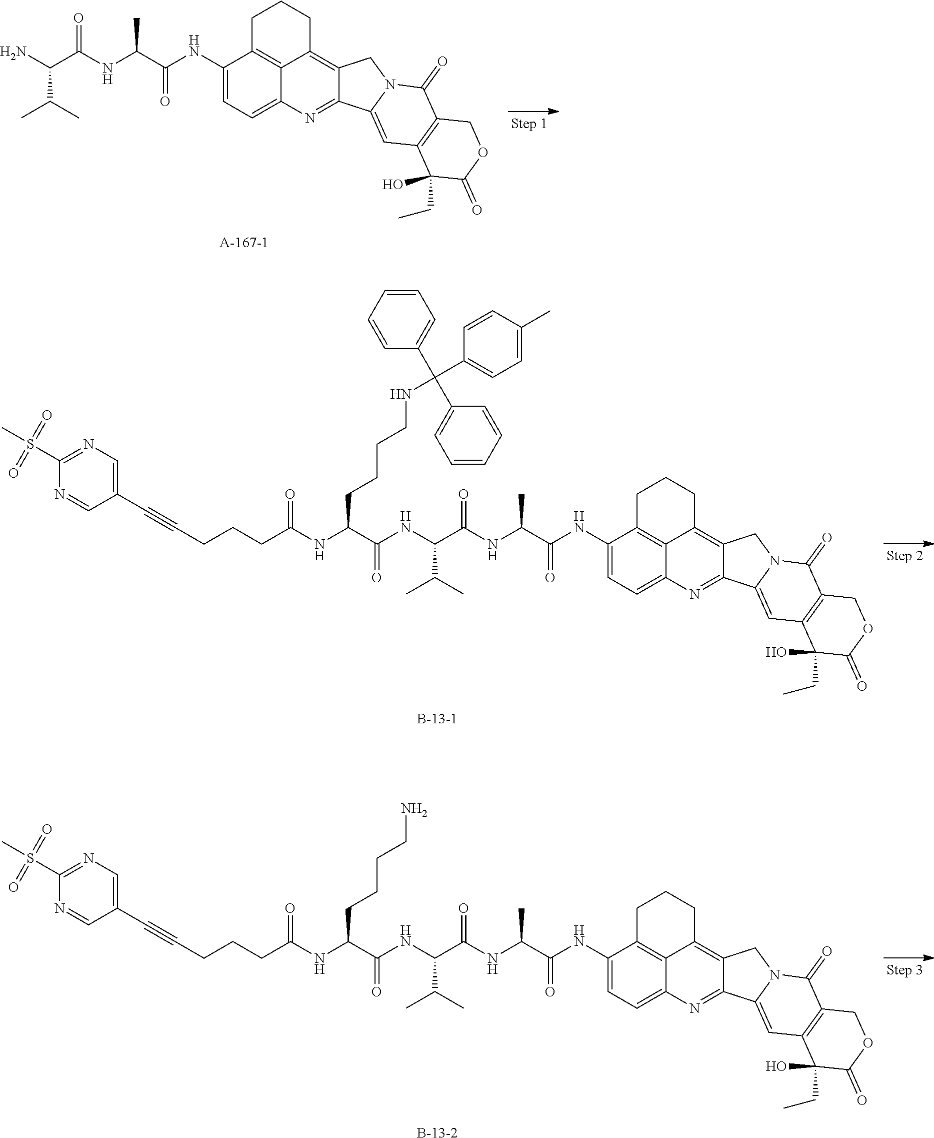 [SMILES: CC[C@@]1(O)C(=O)OCc2c1cc1n(c2=O)Cc2c-1nc1ccc(NC(=O)[C@H](C)NC(=O)[C@@H](N)C(C)C)c3c1c2CCC3 ; "A-167-1" ; CC[C@@]1(O)C(=O)OCc2c1cc1n(c2=O)Cc2c-1nc1ccc(NC(=O)[C@H](C)NC(=O)[C@@H](NC(=O)[C@H](CCCCNC(c3ccccc3)(c3ccccc3)c3ccc(C)cc3)NC(=O)CCCC#Cc3cnc(S(C)(=O)=O)nc3)C(C)C)c3c1c2CCC3 ; "B-13-1" ; CC[C@@]1(O)C(=O)OCc2c1cc1n(c2=O)Cc2c-1nc1ccc(NC(=O)[C@H](C)NC(=O)[C@@H](NC(=O)[C@H](CCCCN)NC(=O)CCCC#Cc3cnc(S(C)(=O)=O)nc3)C(C)C)c3c1c2CCC3 ; "B-13-2"]

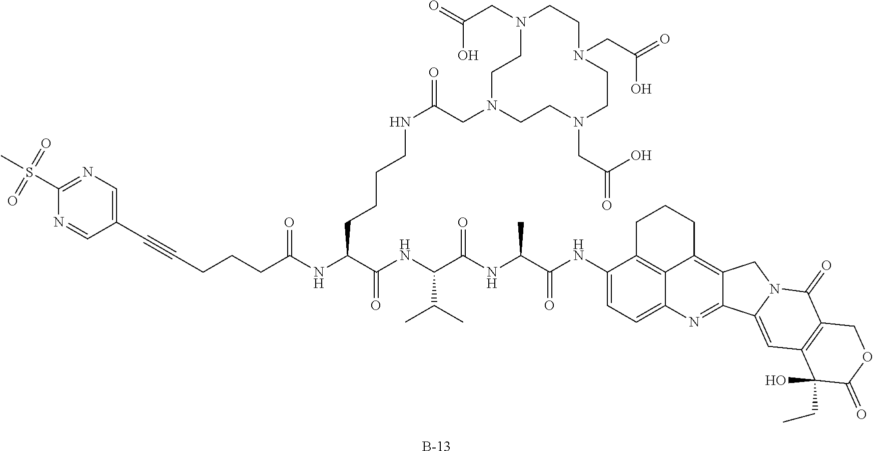

### Step 1: Preparation of N—((S)-6-((diphenyl(p-tolyl)methyl)amino)-1-(((S)-1-(((S)-1-(S) 9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutyl-2-yl)amino)-1-oxohexan-2-yl)-6-(2-(methylsulfonyl)pyrimidin-5-yl)hex-5-ynamide (B-13-1)

(S)-2-Amino-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (20.00 mg, 34.90 μmol) and N⁶-(diphenyl(p-tolyl)methyl)-N²-(6-(2-(methylsulfonyl)pyrimidin-5-yl)hex-5-ynoyl)-L-lysine (25.03 mg, 38.39 μmol) were dissolved in DMF (1 mL), added with 2-(7-azobenzotriazole)-N,N,N′,N′-tetramethyluronium hexafluorophosphate (14.60 mg, 38.39 μmol) and DIPEA (13.50 mg, 104.7 μmol), and stirred at room temperature for 1 hour. After the reaction was completed, the reaction solution was purified by flash column chromatography (C18, water/acetonitrile=2:1) and freeze-dried to obtain the title compound (33 mg, 27.34 μmol).

Its structural characterization data are as follows:

MS m/z (ESI): 1208.4 [M+H]⁺

### Step 2: Preparation of N—((S)-6-amino-1-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutyl-2-yl)amino)-1-oxohex-2-yl)-6-(2-(methylsulfonyl)pyrimidin-5-yl)hex-5-ynamide (B-13-2)

N—((S)-6-((Diphenyl(p-tolyl)methyl)amino)-1-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-1-oxohexan-2-yl)-6-(2-(methylsulfonyl)pyrimidin-5-yl)hexan-5-ynamide (33 mg, 27.34 μmol) was dissolved in DCM (1 mL), added with 1 mL formic acid, and stirred for 1 hour. After the reaction was completed, the reaction solution was purified by flash column chromatography (C18, water/acetonitrile=2:1) and freeze-dried to obtain the title compound (24.0 mg, 25.21 μmol).

Its structural characterization data are as follows:

MS m/z (ESI): 952.2 [M+H]+

### Step 3: Preparation of 2,2′,2″-(10-(2-(((S)-6-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-5-(6-(2-(methylsulfonyl)pyrimidin-5-yl)hex-5-ynamido)-6-oxohexyl)amino)-2-oxoethyl)-1,4,7,10-tetraazacyclododecan-1,4,7-triyl)triacetic acid (B-13)

N—((S)-6-amino-1-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-1-oxohexan-2-yl)-6-(2-(methylsulfonyl)pyrimidin-5-yl)hexan-5-ynamide (24.0 mg, 25.21 μmol) was dissolved in DMF (1 mL), added with DIPEA (9.76 mg, 75.63 μmol), and then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (37.89 mg, 75.63 μmol). After the reaction was completed, the reaction solution was directly purified by preparative high performance liquid chromatography and freeze-dried to obtain the title compound (10.01 mg, 5.15 μmol).

Its structural characterization data are as follows:

MS m/z (ESI): 1338.5 [M+H]⁺

Its preparation method was as follows:

Chromatographic column: Waters Xbridge Prep C18 OBD )
(5 μm*19 mm*150 mm)
Mobile phase A: acetonitrile;
Mobile phase B: water (0.05% formic acid)

| Time | Mobile phase A | Mobile phase B | Flow rate |
|---|---|---|---|
| [min] | [%] | [%] | [mL/min] |
| 0.00 | 20 | 80 | 32 |
| 4.00 | 20 | 80 | 32 |
| 24.00 | 90 | 10 | 32 |

### Example 52: Preparation of 2,2′,2″-(10-((2S,5S,14S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-14-(6-(2-(methylsulfonyl)pyrimidin-5-yl)hex-5-ynamide)-1,4,7,10,13,20-hexaoxo-3,6,9,12,19-pentaazaheneicosan-2l-yl)-1,4,7,10-tetraazacyclododecan-1,4,7-triyl)triacetic acid (B-14)

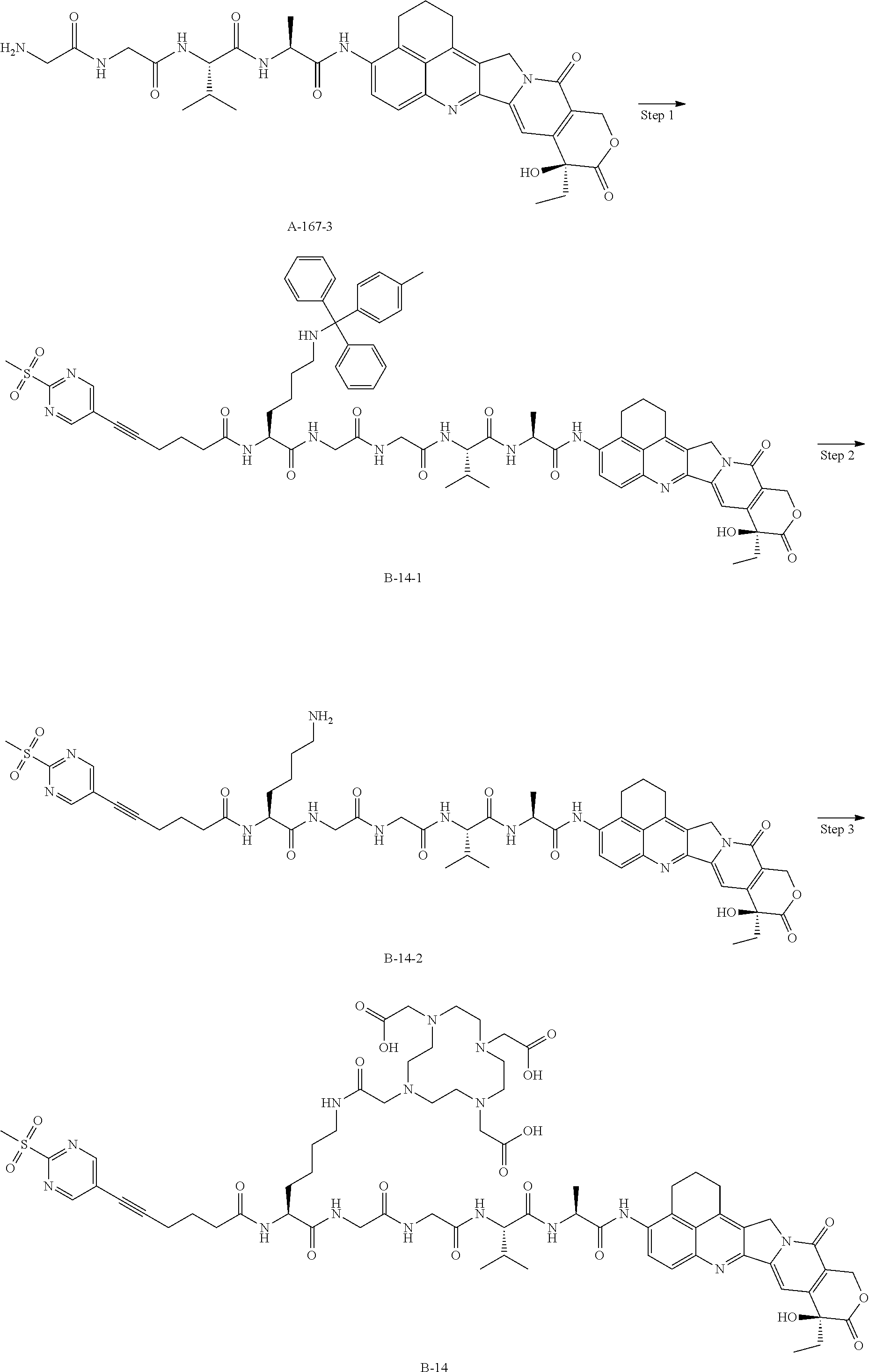

### Step 1: Preparation of N-((7S,16S,19S)-20-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-16-isopropyl-19-methyl-8,11,14,17,20-pentaoxo-1,1-diphenyl-1-(p-tolyl)-2,9,12,15,18-pentaazaeicosan-7-yl)-6-(2-(methylsulfonyl)pyrimidin-5-yl)hex-5-ynamide (B-14-1)

(S)-2-(2-(2-Aminoacetamido)acetamido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (20.00 mg, 29.10 μmol) and N⁶-(diphenyl(p-tolyl) methyl)-N²-(6-(2-(methylsulfonyl)pyrimidin-5-yl)hex-5-ynoyl)-L-lysine (22.00 mg, 32.01 μmol) were dissolved in DMF (1 mL), added with 2-(7-azobenzotriazolyl)-N,N,N′,N′-tetramethyluronium hexafluorophosphate (12.16 mg, 32.01 μmol) and DIPEA (11.26 mg, 87.3 μmol), and stirred at room temperature for 1 hour. After the reaction was completed, the reaction solution was purified by flash column chromatography (C18, water/acetonitrile=2:1) and freeze-dried to obtain the title compound (31 mg, 23.44 μmol).

Its structural characterization data are as follows:

MS m/z (ESI): 1322.4 [M+H]⁺

### Step 2: Preparation of N-((2S,5S,14S)-18-amino-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,10,13-pentaoxo-3,6,9,12-tetraazaoctadecan-14-yl)-6-(2-(methylsulfonyl)pyrimidin-5-yl)hex-5-ynamide (B-14-2)

N-((7S,16S,19S)-20-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-16-isopropyl-19-methyl-8,11,14,17,20-pentaoxo-1,1-diphenyl-1-(p-tolyl)-2,9,12,15,18-pentaazaeicostan-7-yl)-6-(2-(methylsulfonyl)pyrimidin-5-yl)hex-5-ynamide (31 mg, 23.44 μmol) was dissolved in dichloromethane (1 mL), added with 1 mL formic acid, and stirred for 1 hour. After the reaction was completed, the reaction solution was purified by flash column chromatography (C18, water/acetonitrile=2:1) and freeze-dried to obtain the title compound (20.0 mg, 18.76 μmol).

Its structural characterization data are as follows:

MS m/z (ESI): 1066.2 [M+H]⁺

### Step 3: Preparation of 2,2′,2″-(10-((2S,5S,14S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-14-(6-(2-(methylsulfonyl)pyrimidin-5-yl)hex-5-ynamido)-1,4,7,10,13,20-hexaoxo-3,6,9,12,19-pentaazaheneicosan-21-yl)-1,4,7,10-tetraazacyclododecan-1,4,7-triyl)triacetic acid (B-14)

N-((2S,5S,14S)-18-amino-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,10,13-pentaoxo-3,6,9,12-tetraazaoctadecan-14-yl)-6-(2-(methylsulfonyl)pyrimidin-5-yl)hex-5-ynamide (20.0 mg, 18.76 μmol) was dissolved in DMF (1 mL), added with DIPEA (7.26 mg, 56.28 μmol), and added with 2,2′,2″-(10-(2-((2,5-dioxopyrrolidin-1-yl)oxy)-2-oxoethyl)-1,4,7,10-tetraazacyclododecan-1,4,7-triyl)triacetic acid (28.20 mg, 56.28 μmol). After the reaction was completed, the reaction solution was directly purified by preparative high performance liquid chromatography and freeze-dried to obtain the title compound (3.52 mg, 1.68 μmol).

Its structural characterization data are as follows:

MS m/z (ESI): 1452.6 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 20 | 80 | 32 |
| 4.00 | 20 | 80 | 32 |
| 24.00 | 90 | 10 | 32 |

### Example 53: Preparation of 2,2′,2″-(10-((4R,12S,21S)-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-21-(6-(2-(methylsulfonyl)pyrimidin-5-yl)hex-5-ynamido)-3,8,11,14,17,20,27-heptaoxo-5-oxa-2,7,10,13,16,19,26-heptaazaoctacosan-28-yl)-1,4,7,10-tetraazacyclododecan-1,4,7-triyl)triacetic acid (B-15)

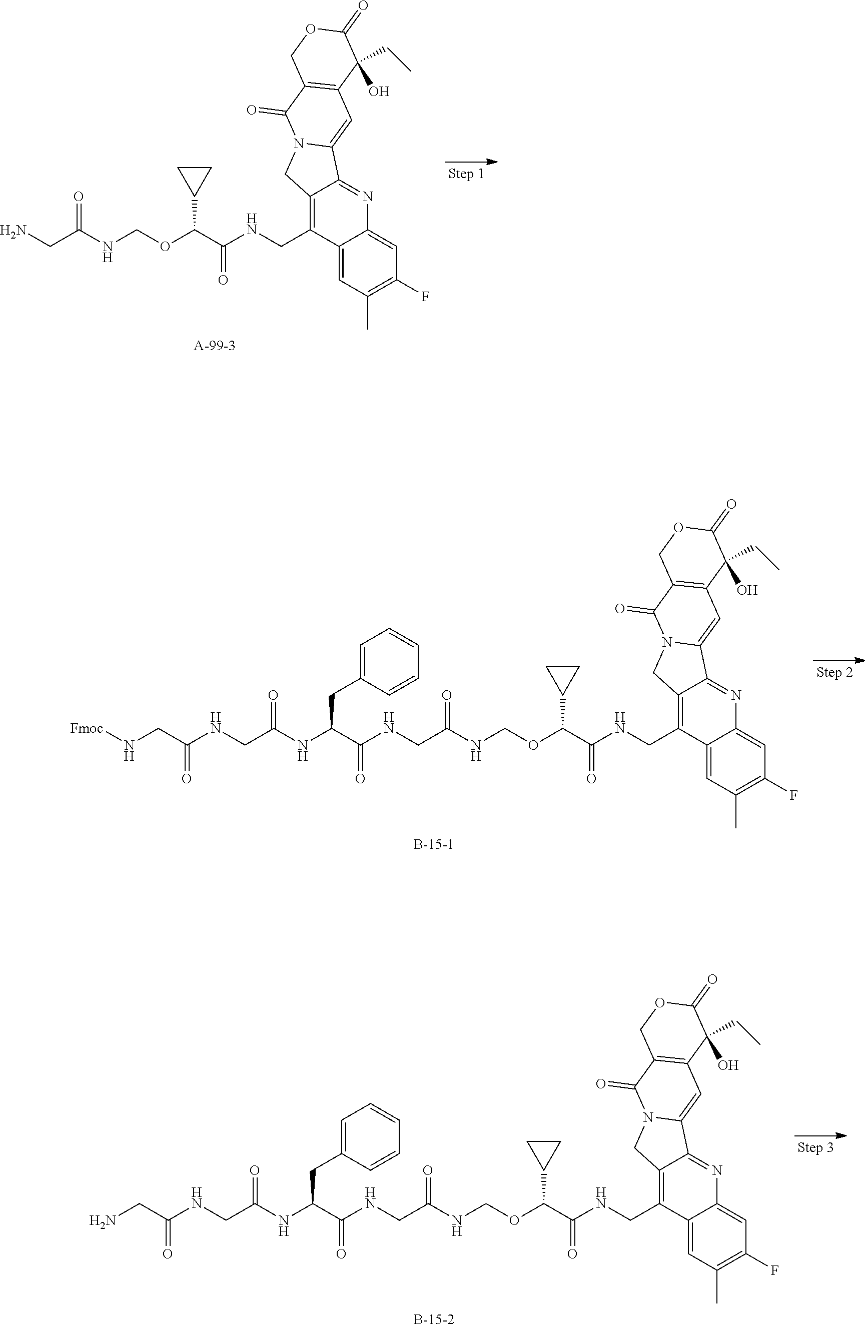

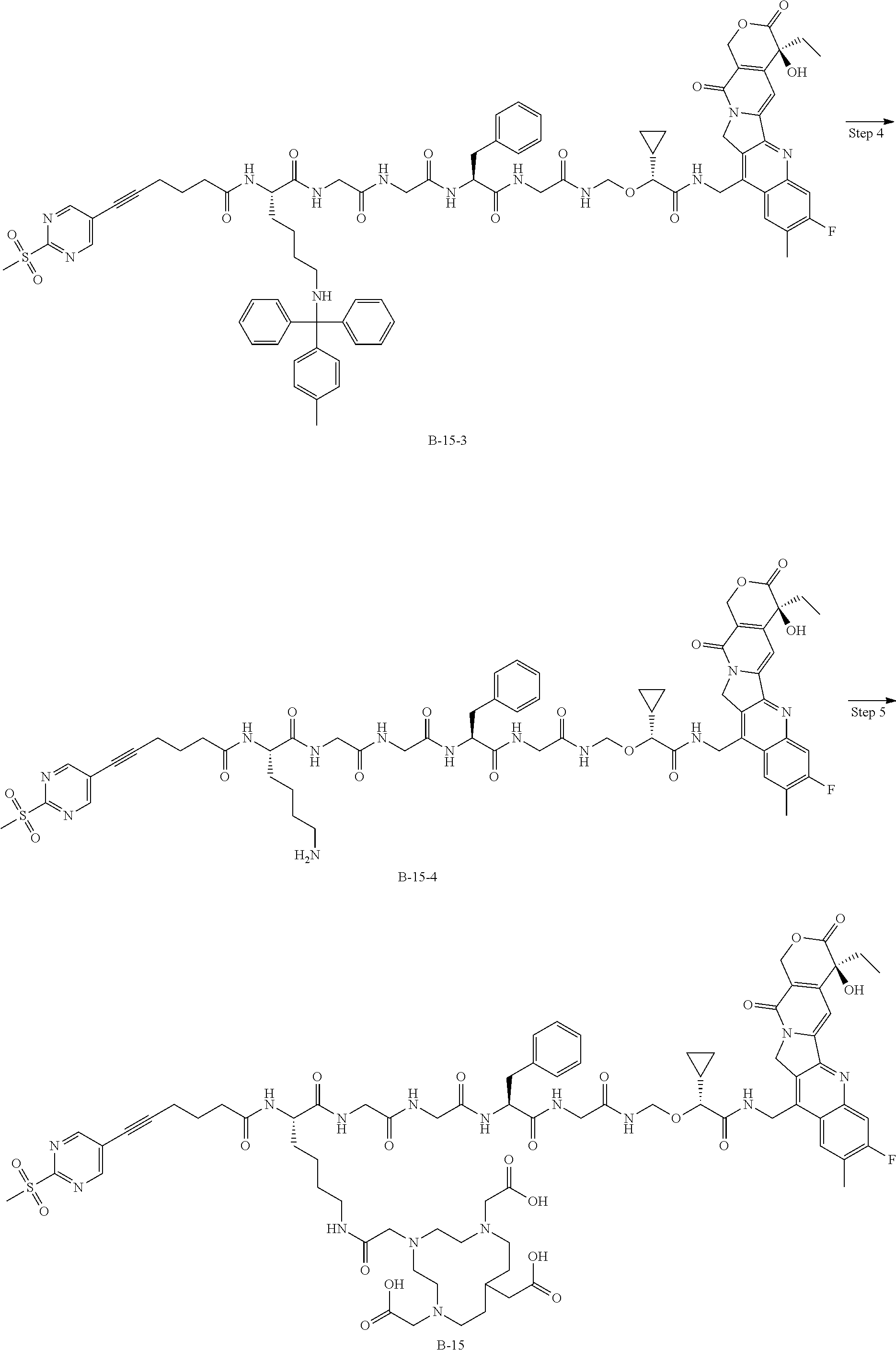

### Step 1: Preparation of (9H-fluoren-9-yl)methyl ((4R,12S)-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)carbamate (B-15-1)

(R)-2-((2-Aminoacetylamino)methoxy)-2-cyclopropyl-N—(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)acetamide (230 mg, 359.58 μmol) was dissolved in N,N-dimethylformamide (10 mL), added with N,N-diisopropylethylamine (232.36 mg, 1.80 mmol), then stirred for 5 minutes, and added with (((9H-fluoren-9-yl)methoxy)carbonyl)glycylglycyl-L-phenylalanine (198.38 mg, 395.54 μmol) and HATU (273.28 mg, 719.17 μmol). The reaction was carried out at 20° C. for 1 hour. After the reaction was completed, the reaction solution was subjected to flash column chromatography (C18, water/acetonitrile=2/1) and freeze-dried to obtain the title compound (210 mg, 194.97 μmol).

Its structural characterization data are as follows:

MS m/z (ESI): 1077.7 [M+H]⁺

### Step 2: Preparation of (S)-2-(2-(2-aminoacetamido)acetamido)-N-(2-((((R)-1-cyclopropyl-2-(((((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)amino)-2-oxoethoxy)methyl)amino)-2-oxoethyl)-3-phenylpropanamide (B-15-2)

(9H-Fluoren-9-yl)methyl ((4R,12S)-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17-pentaoxo-5-oxa-2,7,10,13,16-pentaazaoctadecan-18-yl)carbamic acid (220 mg, 204.25 μmol) was dissolved in N,N-dimethylacetamide (10 mL), and added with diethylamine (74.65 mg, 1.02 mmol). The reaction was carried out at 20° C. for 1 hour. After the reaction was completed, the reaction solution was subjected to flash column chromatography (C18, water/acetonitrile=2/1) and freeze-dried to obtain the title compound (140 mg, 155.40 μmol).

Its structural characterization data are as follows:

MS m/z (ESI): 855.4 [M+H]⁺

### Step 3: Preparation of N-((4R,12S,21S)-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-27,27-diphenyl-27-(p-tolyl)-5-oxa-2,7,10,13,16,19,26-heptaazaheptacosan-21-yl)-6-(2-(methylsulfonyl)pyrimidin-5-yl)hex-5-ynamide (B-15-3)

(S)-2-(2-(2-Aminoacetamido)acetamido)-N-(2-((((R)-1-cyclopropyl-2-((((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)amino)-2-oxoethoxy)methyl)amino)-2-oxoethyl)-3-phenylpropanamide (30 mg, 33.30 μmol) was dissolved in N,N-dimethylformamide (5 mL), added with N,N-diisopropylethylamine (21.52 mg, 166.50 μmol), stirred for 5 minutes, and then added with N⁶-(diphenyl(p-tolyl)methyl)-N²-(6-(2-(methylsulfonyl)pyrimidin-5-yl)hex-5-ynoyl)-L-lysine (32.61 mg, 49.95 μmol) and HATU (25.31 mg, 66.60 μmol). The reaction was carried out at 20° C. for 1 hour. After the reaction was completed, the reaction solution was subjected to flash column chromatography (C18, water/acetonitrile=3/2) and freeze-dried to obtain the title compound (20 mg, 13.43 μmol).

Its structural characterization data are as follows:

MS m/z (ESI): 1489.7 [M+H]⁺

### Step 4: Preparation of N-((4R,12S,21S)-25-amino-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazapentacosan-21-yl)-6-(2-(methylsulfonyl)pyrimidin-5-yl)hex-5-ynamide (B-15-4)

N-((4R,12S,21S)-12-Benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-27,27-diphenyl-27-(p-tolyl)-5-oxa-2,7,10,13,16,19,26-heptaazaheptacosan-21-yl)-6-(2-(methylsulfonyl)pyrimidin-5-yl)hex-5-ynamide (40 mg, 26.85 μmol) was dissolved in dichloromethane (5 mL) and formic acid (2 mL). The reaction was carried out at 20° C. for 3 hours. After the reaction was completed, the reaction solution was subjected to flash column chromatography (C18, water/acetonitrile=3/2) and freeze-dried to obtain the title compound (18 mg, 14.07 μmol).

Its structural characterization data are as follows:

MS m/z (ESI): 1234.7 [M+H]⁺

### Step 5: Preparation of 2,2′,2″-(10-((4R,12S,21S)-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-21-(6-(2-(methylsulfonyl)pyrimidin-5-yl)hex-5-ynamido)-3,8,11,14,17,20,27-heptaoxo-5-oxa-2,7,10,13,16,19,26-heptaazaoctacosan-28-yl)-1,4,7,10-tetraazacyclododecan-1,4,7-triyl)triacetic acid (B-15)

N-((4R,12S,21S)-25-Amino-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazapentacosan-2l-yl)-6-(2-(methylsulfonyl)pyrimidin-5-yl)hex-5-ynamide (18 mg, 14.59 μmol) was dissolved in N,N-dimethylformamide (5 mL), added with N,N-diisopropylethylamine (9.43 mg, 72.97 μmol) and 2,2′,2″-(10-(2-((2,5-dioxopyrrolidin-1-yl)oxy)-2-oxoethyl)-1,4,7,10-tetraazacyclododecan-1,4,7-triyl)triacetic acid (18.30 mg, 36.49 μmol). The reaction was carried out at 20° C. for 1 hour. After the reaction was completed, the reaction solution was directly purified by preparative high performance liquid chromatography and freeze-dried to obtain the title compound (22 mg, 13.02 μmol).

Its structural characterization data are as follows:

MS m/z (ESI): 1620.7 [M+H]⁺

Its preparation method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 30 |
| 2.00 | 10 | 90 | 30 |
| 18.00 | 80 | 20 | 30 |

### Example 54: Preparation of 2,2′,2″-(10-(2-((2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((S)-2-((S)-3-methyl-2-(6-(2-(methylsulfonyl)pyrimidin-5-yl)hex-5-ynamido)butanamido)propionamido)benzyl)(methyl)amino)-2-oxoethyl)-1,4,7,10-tetraazacyclododecan-1,4,7-triyl)triacetic acid (A-198)

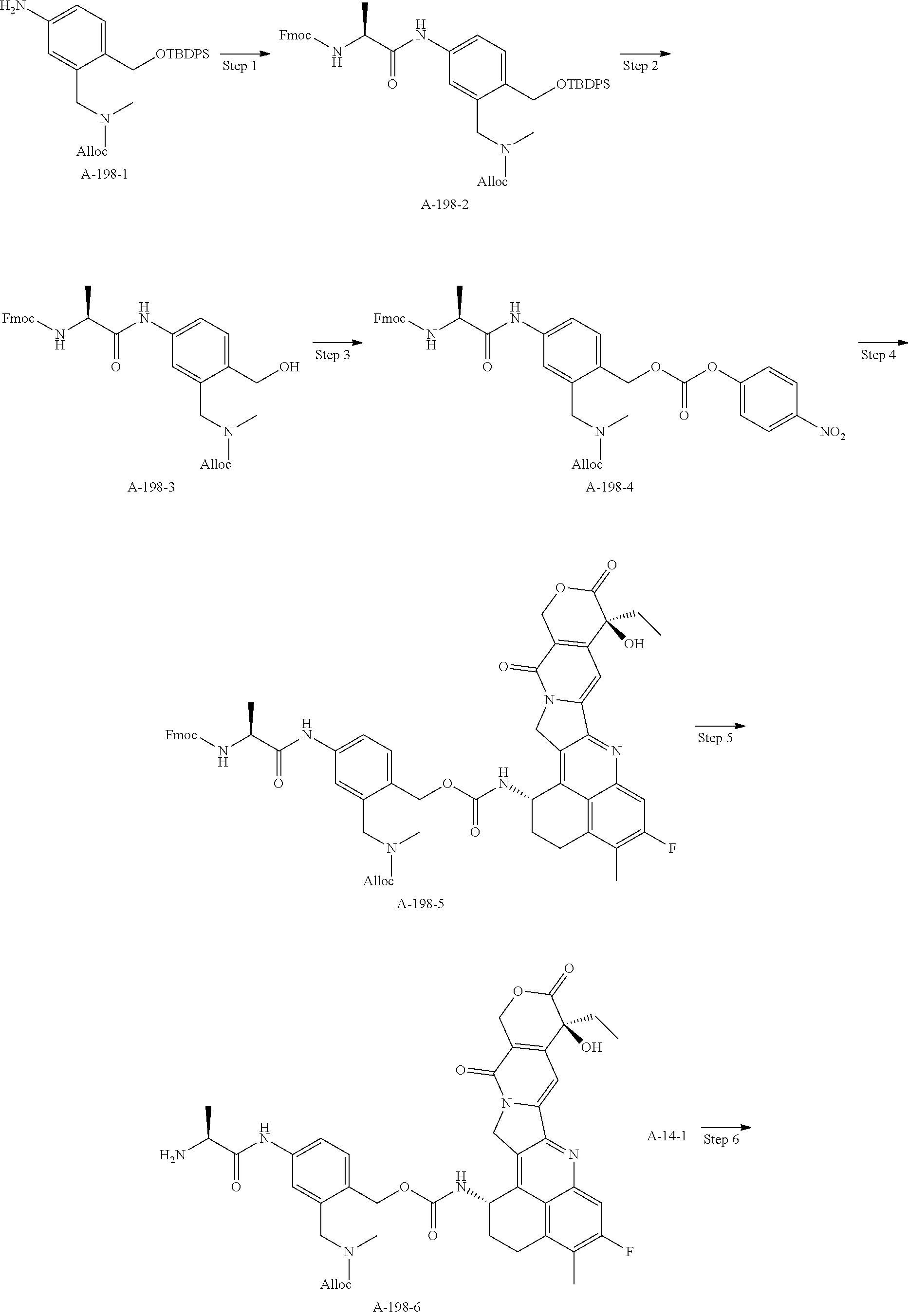

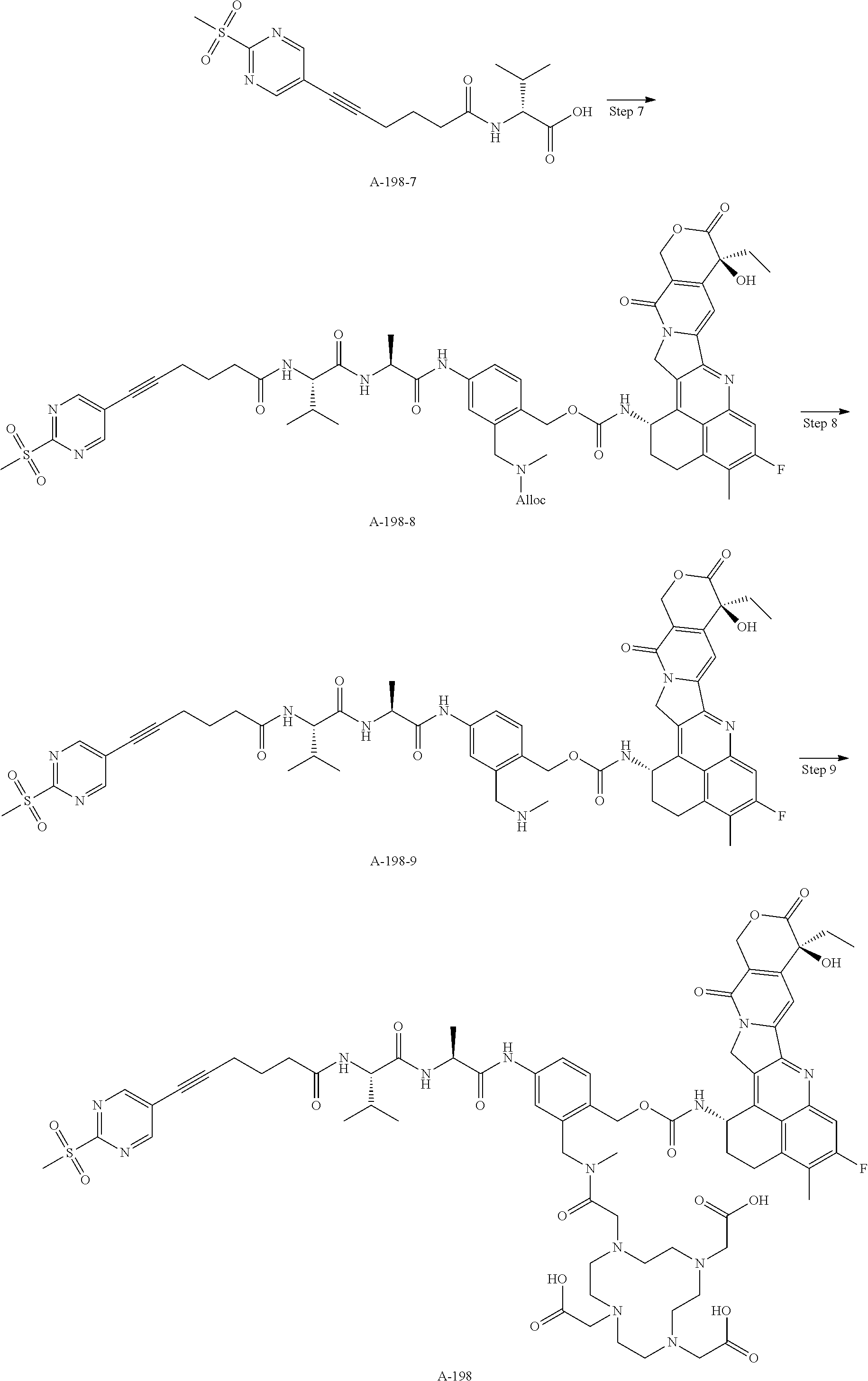

Step 1: Preparation of Allyl (S)-(5-(2-((((9H-fluoren-9-yl)methoxy)carbonyl)amino) propionamido)-2-(((tert-butyldiphenylsilyl)oxy)methyl)benzyl)(methyl)carbamate (A-198-2)

Allyl (5-amino-2-(((tert-butyldiphenylsilyl)oxy)methyl)benzyl)(methyl)carbamate (150.0 mg, 0.31 mmol) and (((9H-fluoren-9-yl)methoxy)carbonyl)-L-alanine (115.1 mg, 0.37 mmol) were dissolved in DCM (8 mL) and MeOH (2 mL), added with 2-ethoxy-1-ethoxycarbonyl-1,2-dihydroquinoline (229.7 mg, 0.93 mmol), and stirred at room temperature for 15 hours. The reaction solution was concentrated under reduced pressure to obtain a crude product, which was purified by silica gel column chromatography (ethyl acetate/petroleum ether=0 to 90%) and concentrated under reduced pressure again to obtain the title compound (194.2 mg, 0.25 mmol).

Its structural characterization data are as follows:

MS m/z (ESI): 782.2 [M+H]⁺

### Step 2: Preparation of (S)-(5-(2-((((9H-fluoren-9-yl)methoxy)carbonyl)amino) propionamido)-2-(hydroxymethyl)benzyl)(methyl)carbamate (A-198-3)

Allyl (S)-(5-(2-((((9H-fluoren-9-yl)methoxy)carbonyl)amino)propionamido)-2-(((tert-butyldiphenylsilyl)oxy)methyl)benzyl)(methyl)carbamate (194.2 mg, 0.25 mmol) was dissolved in DMF (5 mL), added with pyridine hydrofluoride (390.2 mg, 3.93 mmol), and stirred at room temperature for 15 hours. After the reaction was completed, the reaction solution was added with 20 mL of water, extracted with ethyl acetate 3 times (10 mL×3), washed with 10 mL of brine, dried over anhydrous sodium sulfate and concentrated under reduced pressure to obtain the crude product of the title compound, which was purified by flash column chromatography (C18, water/acetonitrile=2/1) and freeze-dried to obtain the title compound (109.3 mg, 0.21 mmol).

Its structural characterization data are as follows:

MS m/z (ESI): 566.1 [M+Na]⁺

### Step 3: Preparation of allyl (S)-(5-(2-((((9H-fluoren-9-yl)methoxy)carbonyl)amino) propionamido)-2-((((4-nitrophenoxy)carbonyl)oxy)methyl)benzyl) (methyl)carbamate (A-198-4)

(S)-(5-(2-((((9H-Fluoren-9-yl)methoxy)carbonyl)amino)propionamido)-2-(hydroxymethyl)benzyl) (methyl)carbamate (109.3 mg, 0.21 mmol) was dissolved in DMF (5 mL), added with DIPEA (81.3 mg, 0.63 mmol), then added with p-nitrophenyl chloroformate (50.8 mg, 0.25 mmol), and stirred at room temperature for 2 hours. After the reaction was completed, the reaction solution was purified by flash column chromatography (C18, water/acetonitrile=2/1) and freeze-dried to obtain the title compound (134.50 mg, 0.19 mmol).

Its structural characterization data are as follows:

MS m/z (ESI): 731.2 [M+Na]⁺

### Step 4: Preparation of allyl (5-((S)-2-((((9H-fluoren-9-yl)methoxy)carbonyl)amino) propionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (A-198-5)

Allyl (S)-(5-(2-((((9H-fluoren-9-yl)methoxy)carbonyl)amino)propionamido)-2-((((4-nitrophenoxy)carbonyl)oxy)methyl)benzyl) (methyl)carbamate (134.50 mg, 0.19 mmol) and (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinolin-10,13-dione (165.50 mg, 0.38 mmol) were dissolved in DMF (5 mL), added with 1-hydroxybenzotriazole (30.80 mg, 0.23 mmol), pyridine (16.5 mg, 0.21 mmol) and DIPEA (73.5 mg, 0.57 mmol), and stirred at room temperature for 2 hours. After the reaction was completed, the reaction solution was purified by flash column chromatography (C18, water/acetonitrile=0.7) and freeze-dried to obtain the title compound (114.70 mg, 0.12 mmol).

Its structural characterization data are as follows:

MS m/z (ESI): 1005.2 [M+H]⁺

### Step 5: Preparation of allyl (5-((S)-2-aminopropionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (A-198-6)

Allyl (5-((S)-2-((((9H-fluoren-9-yl)methoxy)carbonyl)amino)propionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (114.70 mg, 0.12 mmol) was dissolved in DMF (3 mL), added with diethylamine (87.8 mg, 1.2 mmol), and stirred at room temperature for 1 hour. After the reaction was completed, the reaction solution was purified by flash column chromatography (C18, water/acetonitrile=0.7) and freeze-dried to obtain the title compound (62.6 mg, 0.08 mmol).

Its structural characterization data are as follows:

MS m/z (ESI): 783.1 [M+H]⁺

### Step 6: Preparation of (6-(2-(methylsulfonyl)pyrimidin-5-yl)hex-5-ynoyl)-L-valine (A-198-7)

L-valine (73.15 mg, 821.92 μmol) and 2,5-dioxopyrrolidin-1-yl 6-(2-(methylsulfonyl) pyrimidin-5-yl)hex-5-ynoate (100.0 mg, 273.97 μmol) were dissolved in N,N-dimethylformamide (8 mL), and added with N,N-diisopropylethylamine (106.03 mg, 821.92 μmol). The reaction was carried out under stirring at room temperature for 1 hour. After the reaction, the reaction solution was purified by flash column chromatography (C18, water/acetonitrile=2/1) and freeze-dried to obtain the title compound (60 mg, 163.49 μmol).

Its structural characterization data are as follows:

MS m/z (ESI): 367.2 [M+H]⁺

### Step 7: Preparation of (2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((S)-2-((S)-3-methyl-2-(6-(2-(methylsulfonyl)pyrimidin-5-yl)hex-5-ynamido)butanamido)propionamido)benzyl)(methyl)carbamate (A-198-8)

(6-(2-(Methylsulfonyl)pyrimidin-5-yl)hex-5-ynoyl)-L-valine (30 mg, 81.74 μmol) and allyl (5-((S)-2-aminopropionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (63.95 mg, 81.74 μmol) were dissolved in DMF (1 mL), added with 2-(7-azobenzotriazolyl)-N,N,N′,N′-tetramethyluronium hexafluorophosphate (34.17 mg, 89.91 μmol) and DIPEA (31.63 mg, 245.22 μmol), and stirred at room temperature for 1 hour. After the reaction was completed, the reaction solution was purified by flash column chromatography (C18, water/acetonitrile=1/2) and freeze-dried to obtain the title compound (45 mg, 39.75 μmol).

Its structural characterization data are as follows:

MS m/z (ESI): 1132.2 [M+H]⁺

### Step 8: Preparation of 4-((S)-2-((S)-3-methyl-2-(6-(2-(methylsulfonyl)pyrimidin-5-yl) hex-5-ynamido)butanamido)propionamido)-2-((methylamino)methyl)benzyl((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (A-198-9)

(2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((S)-2-((S)-3-methyl-2-(6-(2-(methylsulfonyl)pyrimidin-5-yl)hex-5-ynamido)butanamido)propanamido)benzyl)(methyl)carbamate (45 mg, 39.75 μmol) was dissolved in DMF (1 mL), add with tetratriphenylphosphine palladium (4.6 mg, 3.98 μmol), then added with 15p of formic acid, 30 μL of N-methylmorpholine, and stirred at room temperature for 1 hour under nitrogen protection. After the reaction was completed, the reaction solution was purified by flash column chromatography (C18, water/acetonitrile=4/1) and freeze-dried to obtain the title compound (27 mg, 25.76 μmol).

Its structural characterization data are as follows:

MS m/z (ESI): 1048.4 [M+H]⁺

### Step 9: Preparation of 2,2′,2″-(10-(2-((2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((S)-2-((S)-3-methyl-2-(6-(2-(methylsulfonyl)pyrimidin-5-yl)hex-5-ynamido)butanamido)propionamido)benzyl)(methyl)amino)-2-oxoethyl)-1,4,7,10-tetraazacyclododecan-1,4,7-triyl)triacetic acid (A-198)

4-((S)-2-((S)-3-Methyl-2-(6-(2-(methylsulfonyl)pyrimidin-5-yl)hex-5-ynamido)butanamido)propionamido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (27 mg, 25.76 μmol) was dissolved in DMF (1 mL), added with DIPEA (9.97 mg, 77.28 μmol), and then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (38.76 mg, 77.28 μmol). After the reaction was completed, the reaction solution was directly purified by preparative high performance liquid chromatography and freeze-dried to obtain the title compound (3.68 mg, 2.56 μmol).

Its structural characterization data are as follows:

MS m/z (ESI): 1434.6 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 32 |
| 4.00 | 10 | 90 | 32 |
| 24.00 | 90 | 10 | 32 |

### Example 55: Preparation of 2,2′,2″-(10-(2-((2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((S)-2-((S)-3-methyl-2-(6-(2-(methylsulfonyl)pyrimidin-5-yl)hex-5-ynamido)butanamido)propionamido)benzyl)(methyl)amino)-2-oxoethyl)-1,4,7,10-tetraazacyclododecan-1,4,7-triyl)triacetic acid (A-199)

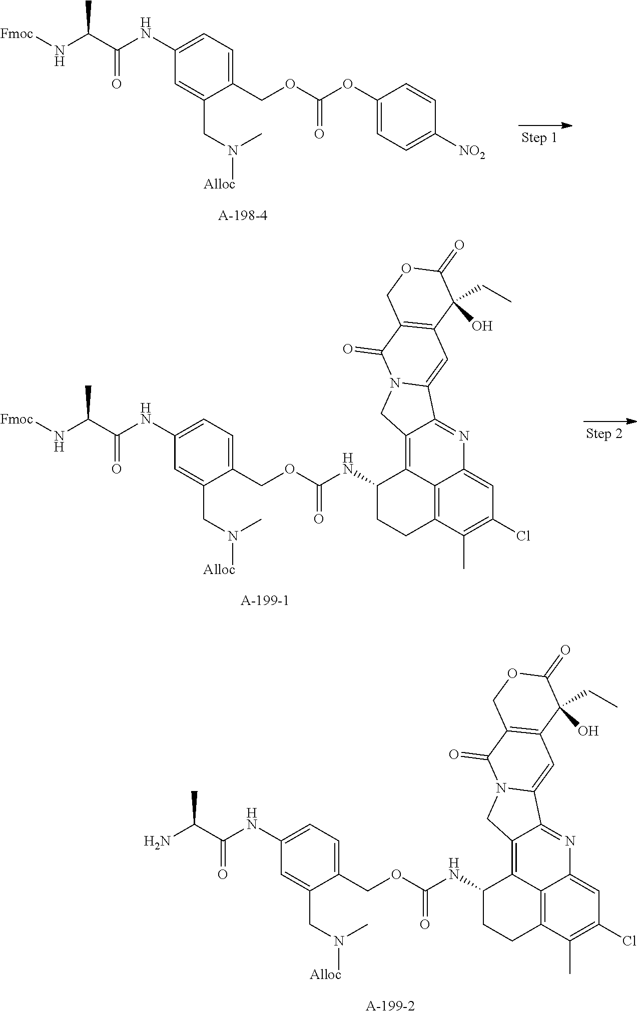

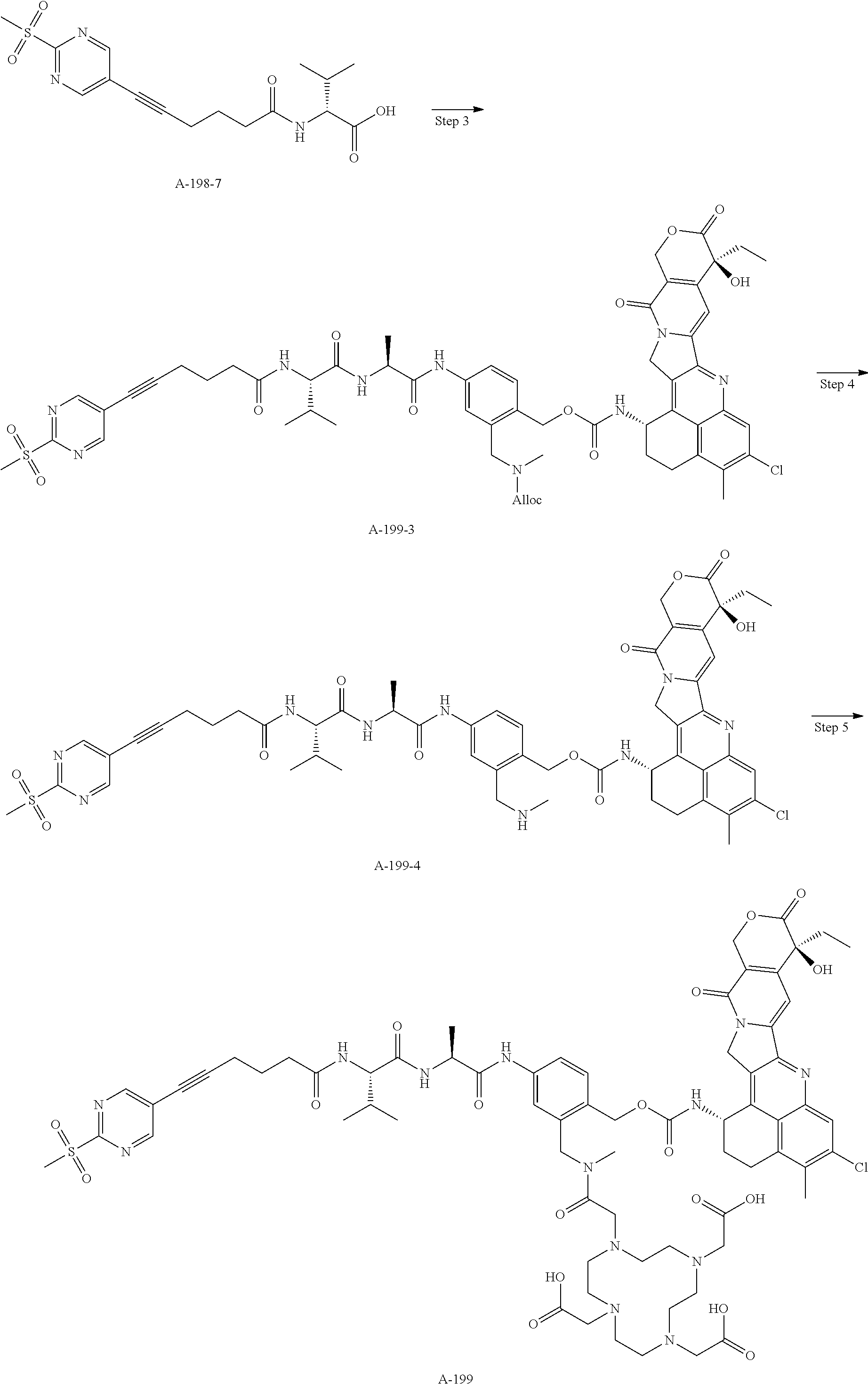

### Step 1: Preparation of allyl (5-((S)-2-((((9H-fluoren-9-yl)methoxy)carbonyl)amino) propionamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)methyl)carbamate (A-199-1)

Allyl (S)-(5-(2-((((9H-fluoren-9-yl)methoxy)carbonyl)amino)propionamido)-2-((((4-nitrophenoxy)carbonyl)oxy)methyl)benzyl)(methyl)carbamate (200.0 mg, 0.28 mmol) and (1S,9S)-1-amino-9-ethyl-5-chloro-9-hydroxy-4-methyl-1,2,3,9,12,15-hexahydro-10H,13H-benzo[de]pyrano[3′,4′:6,7]indolizino[1,2-b]quinolin-10,13-dione (253.0 mg, 0.56 mmol) were dissolved in DMF (6 mL), added with 1-hydroxybenzotriazole (45.5 mg, 0.34 mmol), pyridine (24.5 mg, 0.31 mmol) and DIPEA (108.5 mg, 0.57 mmol), and stirred at room temperature for 2 hours. After the reaction was completed, the reaction solution was purified by flash column chromatography (C18, water/acetonitrile=0.7) and freeze-dried to obtain the title compound (183.70 mg, 0.18 mmol).

Its structural characterization data are as follows:

MS m/z (ESI): 1022.2 [M+H]⁺

### Step 2: Preparation of allyl (5-((S)-2-aminopropionamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (A-199-2)

Allyl (5-((S)-2-((((9H-fluoren-9-yl)methoxy)carbonyl)amino)propionamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)methyl)carbamate (183.70 mg, 0.18 mmol) was dissolved in DMF (3 mL), added with diethylamine (131.8 mg, 1.8 mmol), and stirred at room temperature for 1 hour. After the reaction was completed, the reaction solution was purified by flash column chromatography (C18, water/acetonitrile=0.6) and freeze-dried to obtain the title compound (136.6 mg, 0.17 mmol).

Its structural characterization data are as follows:

MS m/z (ESI): 800.1 [M+H]⁺

### Step 3: Preparation of (2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((S)-2-((S)-3-methyl-2-(6-(2-(methylsulfonyl)pyrimidin-5-yl)hex-5-ynamido)butanamido)propionamido)benzyl)(methyl)carbamate (A-199-3)

(6-(2-(Methylsulfonyl)pyrimidin-5-yl)hex-5-ynoyl)-L-valine (30 mg, 81.74 μmol) and allyl (5-((S)-2-aminopropionamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (65.23 mg, 81.74 μmol) were dissolved in DMF (1 mL), added with 2-(7-azobenzotriazolyl)-N,N,N′,N′-tetramethyluronium hexafluorophosphate (34.17 mg, 89.91 μmol) and DIPEA (31.63 mg, 245.22 μmol), and stirred at room temperature for 1 hour. After the reaction was completed, the reaction solution was purified by flash column chromatography (C18, water/acetonitrile=1/2) and freeze-dried to obtain the title compound (35 mg, 30.50 μmol).

Its structural characterization data are as follows:

MS m/z (ESI): 1147.4 [M+H]⁺

### Step 4: Preparation of 4-((S)-2-((S)-3-methyl-2-(6-(2-(methylsulfonyl)pyrimidin-5-yl) hex-5-ynamido)butanamido)propionamido)-2-((methylamino)methyl)benzyl ((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (A-199-4)

(2-(((((1S,9S)-5-Chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((S)-2-((S)-3-methyl-2-(6-(2-(methylsulfonyl)pyrimidin-5-yl)hex-5-ynamido)butanamido)propionamido)benzyl)(methyl)carbamate (35 mg, 30.50 μmol) was dissolved in DMF (1 mL), added with tetratriphenylphosphine palladium (3.5 mg, 3.05 μmol), added with 15 μL of formic acid and 30 μL of N-methylmorpholine, and stirred at room temperature for 1 hour under nitrogen protection. After the reaction was completed, the reaction solution was purified by flash column chromatography (C18, water/acetonitrile=4/1) and freeze-dried to obtain the title compound (18 mg, 16.93 μmol).

Its structural characterization data are as follows:

MS m/z (ESI): 1063.4 [M+H]⁺

### Step 5: Preparation of 2,2′,2″-(10-(2-((2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((S)-2-((S)-3-methyl-2-(6-(2-(methylsulfonyl)pyrimidin-5-yl)hex-5-ynamido)butanamido)propionamido)benzyl)(methyl)amino)-2-oxoethyl)-1,4,7,10-tetraazacyclododecan-1,4,7-triyl)triacetic acid (A-199)

4-((S)-2-((S)-3-Methyl-2-(6-(2-(methylsulfonyl)pyrimidin-5-yl)hex-5-ynamido)butanamido)propionamido)-2-((methylamino)methyl)benzyl ((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (18 mg, 16.93 μmol) was dissolved in DMF (1 mL), added with DIPEA (6.55 mg, 50.78 μmol), then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (25.47 mg, 50.78 μmol). After the reaction was completed, the reaction solution was directly purified by preparative high performance liquid chromatography and freeze-dried to obtain the title compound (2.77 mg, 2.56 μmol).

Its structural characterization data are as follows:

MS m/z (ESI): 1451.0 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 32 |
| 4.00 | 10 | 90 | 32 |
| 24.00 | 90 | 10 | 32 |

### Example 56: Preparation of 2,2′,2″-(10-(2-(((S)-6-(((S)-1-(((S)-1-((4-((((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxopropan-2-yl)amino)-3-methyl-1-oxobutan-2-yl)amino)-5-(6-(2-(methylsulfonyl)pyrimidin-5-yl)hex-5-ynamido)-6-oxohexyl)amino)-2-oxoethyl)-1,4,7,10-tetraazacyclododecan-1,4,7-triyl)triacetic acid (B-17)

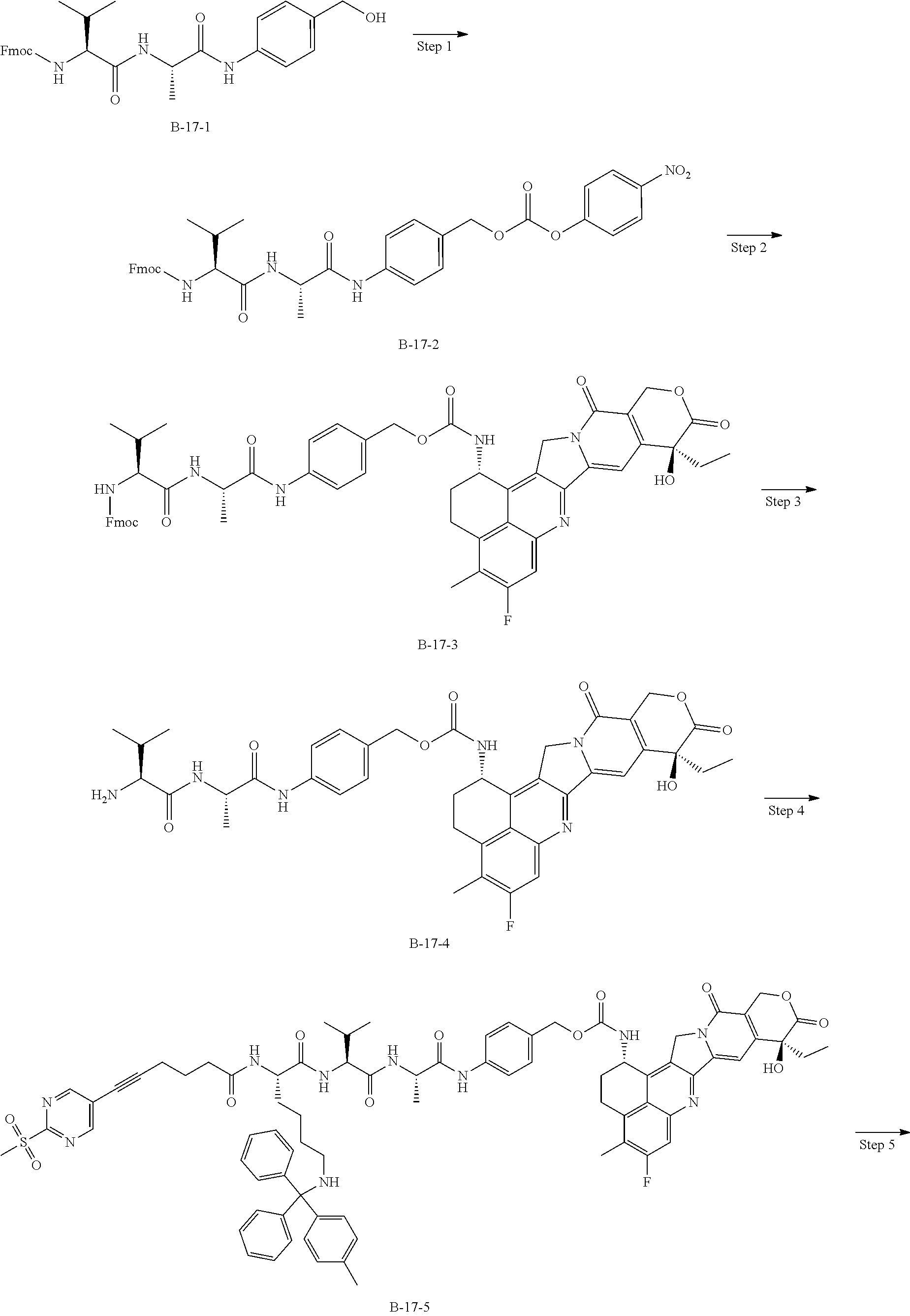

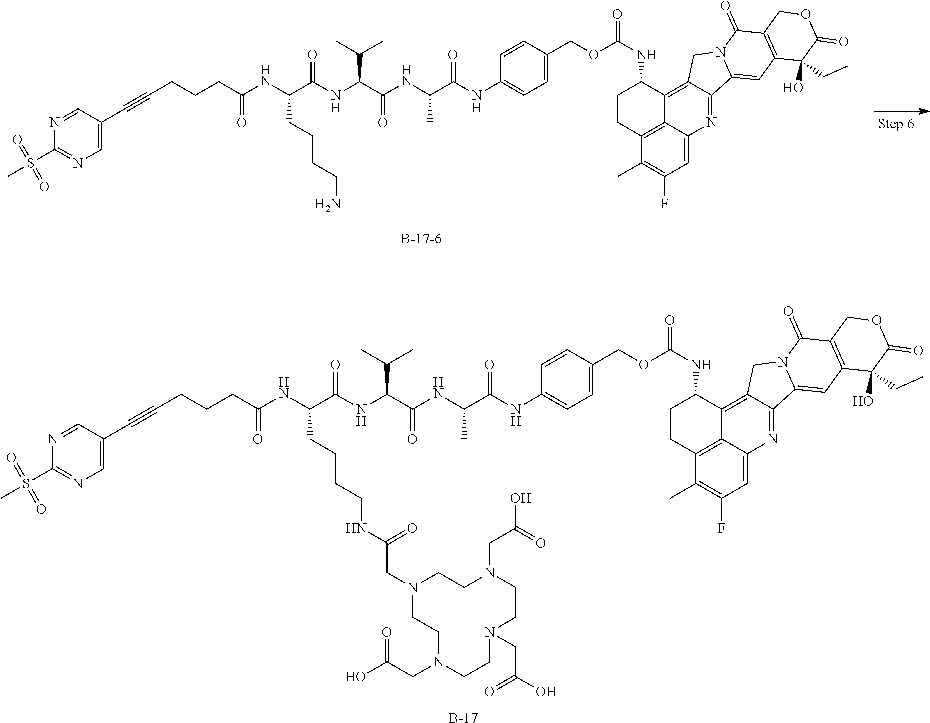

### Step 1: Preparation of (9H-fluoren-9-yl)methyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (B-17-2)

(9H-Fluoren-9-yl)methyl ((S)-1-(((S)-1-((4-(hydroxymethyl)phenyl)amino)-1-oxopropan-2-yl)amino)-3-1-oxobutan-2-yl)carbamate (100.0 mg, 194.17 μmol) and bis(4-nitrophenyl)carbonate (177.09 mg, 582.52 μmol) were dissolved in N,N-dimethylformamide (2 mL), and added with N,N-diisopropylethylamine (75.14 mg, 582.52 μmol). The reaction was carried out under stirring at room temperature for 1 hour. After the reaction was completed, the reaction solution was purified by flash column chromatography (C18, water/acetonitrile=1/4) and freeze-dried to obtain the title compound (80 mg, 117.53 μmol).

Its structural characterization data are as follows:

MS m/z (ESI): 681.2 [M+H]⁺

### Step 2: Preparation of 4-((S)-2-((S)-2-((((9H-fluoren-9-yl)methoxy)carbonyl)amino)-3-methylbutanamido)propionamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (B-17-3)

(9H-Fluoren-9-yl)methyl ((S)-3-methyl-1-(((S)-1-((4-((((4-nitrophenoxy)carbonyl)oxy) methyl)phenyl)amino))-1-oxopropyl-2-yl)amino)-1-oxobutyl-2-yl)carbamate (40 mg, 58.76 μmol) and (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinolin-10,13-dione methanesulfonate (34.36 mg, 64.64 μmol) was dissolved in DMF (1 mL), and added with N,N-diisopropylethylamine (22.74 mg, 176.28 μmol). The reaction was carried out under stirring at room temperature for 1 hour. After the reaction was completed, the reaction solution was purified by flash column chromatography (C18, water/acetonitrile=1/2) and freeze-dried to obtain the title compound (36 mg, 36.87 μmol).

Its structural characterization data are as follows:

MS m/z (ESI): 977.4 [M+H]⁺

### Step 3: Preparation of benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (B-17-4)

4-((S)-2-((S)-2-((((9H-fluoren-9-yl)methoxy)carbonyl)amino)-3-methylbutanamido)propionamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (36 mg, 36.87 μmol) was dissolved in DMF (1 mL), added with diethylamine (1 mL), and stirred at room temperature for 1 hour. After the reaction was completed, the reaction solution was purified by flash column chromatography (C18, water/acetonitrile=4/1) and freeze-dried to obtain the title compound (24 mg, 31.78 μmol).

Its structural characterization data are as follows:

MS m/z (ESI): 755.3 [M+H]⁺

### Step 4: Preparation of 4-((S)-2-((S)-2-((S)-6-((diphenyl(p-tolyl)methyl)amino)-2-(6-(2-(methylsulfonyl)pyrimidin-5-yl)hex-5-ynamido)hexamido)-3-methylbutanamido)propionamido)benzyl ((1S,9S)-9-dioxo-2,3,9,10,13,15-hexahydro-1H,12H-benzo[de]pyrano[3′,4′:6,7]indolizino [1,2-b]quinolin-1-yl)carbamate (B-17-5)

Benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (24 mg, 31.78 μmol) and B-8-6 (22.80 mg, 34.96 μmol) were dissolved in DMF (1 mL), added with 2-(7-azobenzotriazole)-N,N,N′,N′-tetramethyluronium hexafluorophosphate (13.28 mg, 34.96 μmol) and DIPEA (12.30 mg, 95.34 μmol), and stirred at room temperature for 1 hour. After the reaction was completed, the reaction solution was purified by flash column chromatography (C18, water/acetonitrile=1/4) and freeze-dried to obtain the title compound (23 mg, 16.56 μmol).

Its structural characterization data are as follows:

MS m/z (ESI): 1389.6 [M+H]⁺

### Step 5: Preparation of ((1S,9S)-9-ethyl-5-fluoro-9-hydroxy-4-methyl-10,13-dioxo-2-(6-(2-(methylsulfonyl)pyrimidin-5-yl)hex-5-ynamido)hexanamido)-3-methylbutanamido)propanamido)benzyl ((1S,9S)-9-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (B-17-6)

4-((S)-2-((S)-2-((S)-6-((Diphenyl(p-tolyl)methyl)amino)-2-(6-(2-(methylsulfonyl)pyrimidin-5-yl)hex-5-ynamino)hexanamido)-3-methylbutanamido)propanamido)benzyl ((1S,9S)-9-dioxo-2,3,9,10,13,15-hexahydro-1H,12H-benzo[de]pyrano[3′,4′:6,7]indolizino[1,2-b]quinolin-1-yl)carbamate (23 mg, 16.56 μmol) was dissolved in DMF (1 mL), added with formic acid (1 mL), and stirred at room temperature for 1 hour. After the reaction was completed, the reaction solution was purified by flash column chromatography (C18, water/acetonitrile=1/2) and freeze-dried to obtain the title compound (15 mg, 13.24 μmol).

Its structural characterization data are as follows:

MS m/z (ESI): 1133.3 [M+H]⁺

### Step 6: Preparation of 2,2′,2″-(10-(2-(((S)-6-(((S)-1-(((S)-1-((4-((((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxopropan-2-yl)amino)-3-methyl-1-oxobutan-2-yl)amino)-5-(6-(2-(methylsulfonyl)pyrimidin-5-yl)hex-5-ynamide)-6-oxohexyl)amino)-2-oxoethyl)-1,4,7,10-tetraazacyclododecan-1,4,7-triyl)triacetic acid (B-17)

((1S,9S)-9-Ethyl-5-fluoro-9-hydroxy-4-methyl-10,13-dioxo-2-(6-(2-(methylsulfonyl)pyrimidin-5-yl)hex-5-ynamido)hexanamido)-3-methylbutanamido)propanamido)benzyl ((1S,9S)-9-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (15 mg, 13.24 μmol) was dissolved in DMF (1 mL), added with DIPEA (5.12 mg, 39.72 μmol), and added with 2,2′,2″-(10-(2-((2,5-dioxopyrrolidin-1-yl)oxy)-2-oxoethyl)-1,4,7,10-tetraazacyclododecan-1,4,7-triyl)triacetic acid (19.90 mg, 39.72 μmol). After the reaction was completed, the reaction solution was directly purified by preparative high performance liquid chromatography and freeze-dried to obtain the title compound (4.29 mg, 2.79 μmol).

Its structural characterization data are as follows:

MS m/z (ESI): 1519.7 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 32 |
| 4.00 | 10 | 90 | 32 |
| 24.00 | 90 | 10 | 32 |

### Example 57: Preparation of 2,2′,2″-(10-((4R,12S,21S)-12-benzyl-30-carboxyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-21-(6-(2-(methylsulfonyl)pyrimidin-5-yl)hex-5-ynamido)-3,8,11,14,17,20,27-heptaoxo-5-oxa-2,7,10,13,16,19,26-heptaazatriacontan-30-yl)-1,4,7,10-tetraazacyclododecan-1,4,7-triyl)triacetic acid (B-16)

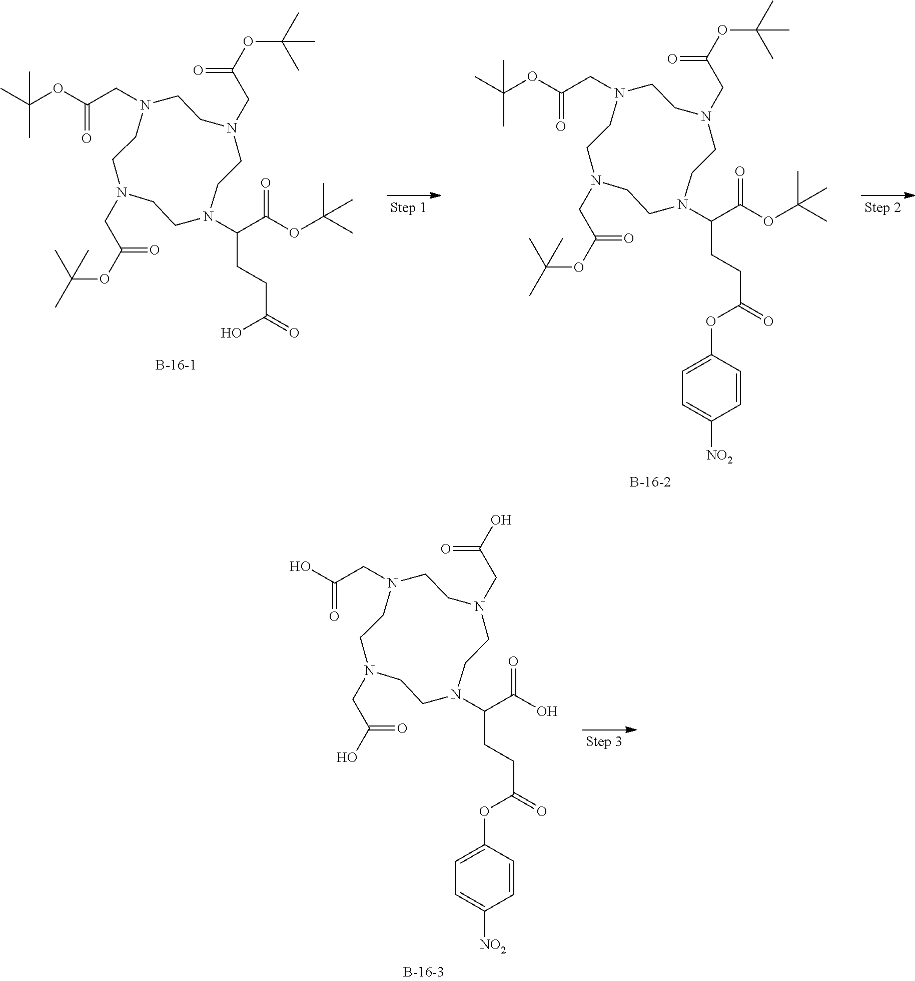 [SMILES: CC(C)(C)OC(=O)CN1CCN(CC(=O)OC(C)(C)C)CCN(C(CCC(=O)O)C(=O)OC(C)(C)C)CCN(CC(=O)OC(C)(C)C)CC1 ; CC(C)(C)OC(=O)CN1CCN(CC(=O)OC(C)(C)C)CCN(C(CCC(=O)Oc2ccc([N+](=O)[O-])cc2)C(=O)OC(C)(C)C)CCN(CC(=O)OC(C)(C)C)CC1 ; O=C(O)CN1CCN(CC(=O)O)CCN(C(CCC(=O)Oc2ccc([N+](=O)[O-])cc2)C(=O)O)CCN(CC(=O)O)CC1]

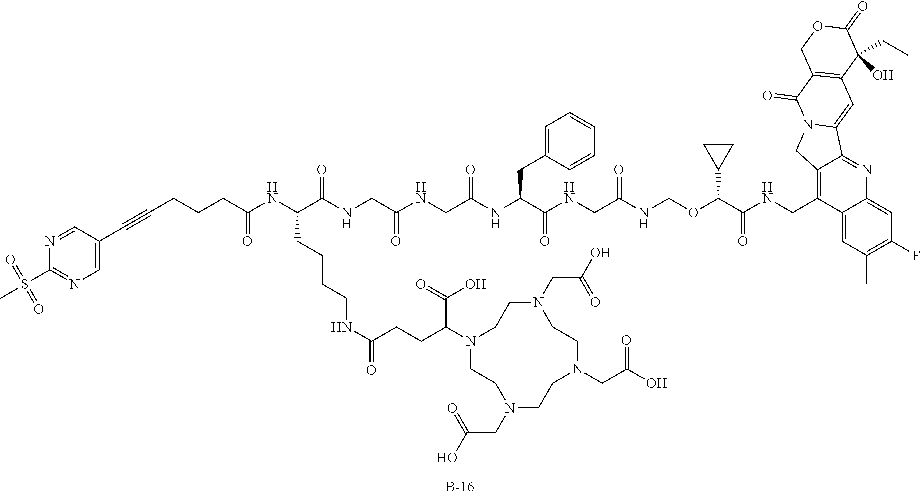

### Step 1: Preparation of 5-(4-nitrophenyl) 2-(4,7,10-tris(2-(tert-butoxy)-2-oxoethyl)-1,4,7,10-tetraazacyclododecan-1-pentanedioic acid-1-tert-butyl ester (B-16-2)

5-(Tert-butoxy)-5-oxo-4-(4,7,10-tris(2-(tert-butoxy)-2-oxoethyl)-1,4,7,10-tetraazacyclododecan-1-yl)pentanoic acid (500 mg, 713.37 μmol) was dissolved in DCM (10 mL), added with p-nitrophenol (90.21 mg, 648.52 μmol) and EDCI (124.32 mg, 648.52 μmol), and reacted at room temperature for 4 hours. The reaction solution was added with water (20 mL) and then separated, the aqueous phase was extracted twice with DCM (20 mL×2), the organic phases were combined, washed with saturated sodium chloride aqueous solution, dried over anhydrous sodium sulfate, and concentrated under vacuum to obtain the title compound (700 mg, 596.11 μmol).

Its structural characterization data are as follows:

MS m/z (ESI): 822.7 [M+H]⁺

### Step 2: Preparation of 2,2′,2″-(10-(1-carboxy-4-(4-nitrophenoxy)-4-oxobutyl)-1,4,7,10-tetraazacyclododecan-1,4,7-triyl)triacetic acid (B-16-1)

5-(4-Nitrophenyl) 2-(4,7,10-tris(2-(tert-butyloxy)-2-oxoethyl)-1,4,7,10-tetraaza-cyclododecan-1-pentanedioic acid-1-tert-butyl ester (100 mg, 121.66 μmol) was dissolved in dichloromethane (5 mL), added with trifluoroacetic acid (5 mL), and reacted at 30° C. for 6 hours. The reaction solution was directly concentrated to obtain the title compound as crude product (70 mg, 117.14 μmol), which was used directly in the next step without purification.

Its structural characterization data are as follows:

MS m/z (ESI): 598.6 [M+H]⁺

### Step 3: Preparation of 2,2′,2″-(10-((4R,12S,21S)-12-benzyl-30-carboxyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-21-(6-(2-(methylsulfonyl)pyrimidin-5-yl)hex-5-ynamido)-3,8,11,14,17,20,27-heptaoxo-5-oxa-2,7,10,13,16,19,26-heptaazatriacontan-30-yl)-1,4,7,10-tetraazacyclododecan-1,4,7-triyl)triacetic acid (B-16)

N-((4R,12S,21S)-25-Amino-12-benzyl-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,8,11,14,17,20-hexaoxo-5-oxa-2,7,10,13,16,19-hexaazapentacosan-2l-yl)-6-(2-(methylsulfonyl)pyrimidin-5-yl)hex-5-ynamide (10 mg, 8.11 μmol) was dissolved in N,N-dimethylformamide (5 mL), added with N,N-diisopropylethylamine (5.24 mg, 40.54 μmol) and crude product of 2,2′,2″-(10-(1-carboxy-4-(4-nitrophenoxy)-4-oxobutyl)-1,4,7,10-tetraazacyclododecan-1,4,7-triyl)triacetic acid (70.00 mg, 117.14 μmol), and reacted at 20° C. for 1 hour. The reaction solution was directly purified by preparative high performance liquid chromatography and freeze-dried to obtain the title compound (2.82 mg, 1.50 μmol).

Its structural characterization data are as follows:

MS m/z (ESI): 1233.5 [M+H]⁺

Its preparation method was as follows:

Chromatographic column: Waters SunFire Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 20 | 80 | 30 |
| 4.00 | 20 | 80 | 30 |
| 24.00 | 90 | 10 | 30 |

### Example 58: Preparation of 2,2′,2″-(10-(2-((2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)amino)-2-oxoethyl)-1,4,7,10-tetraazacyclododecan-1,4,7-triyl)triacetic acid (B-20)

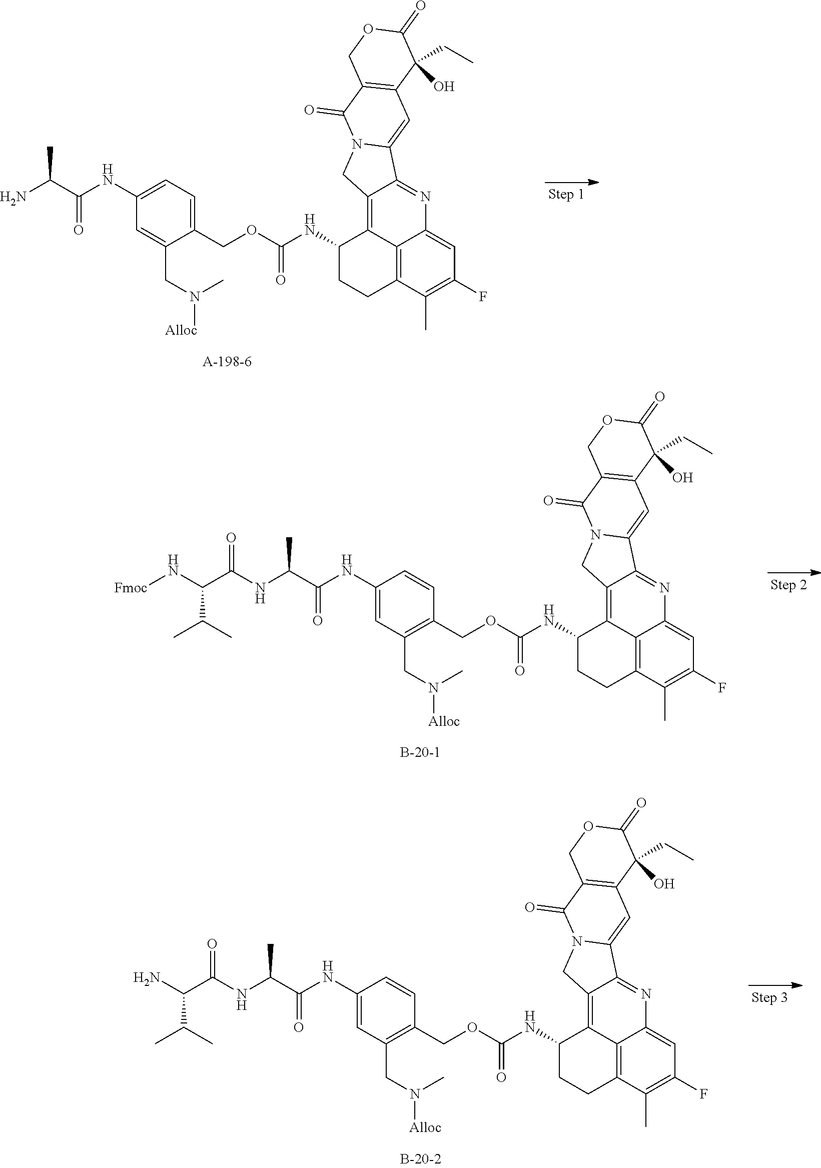

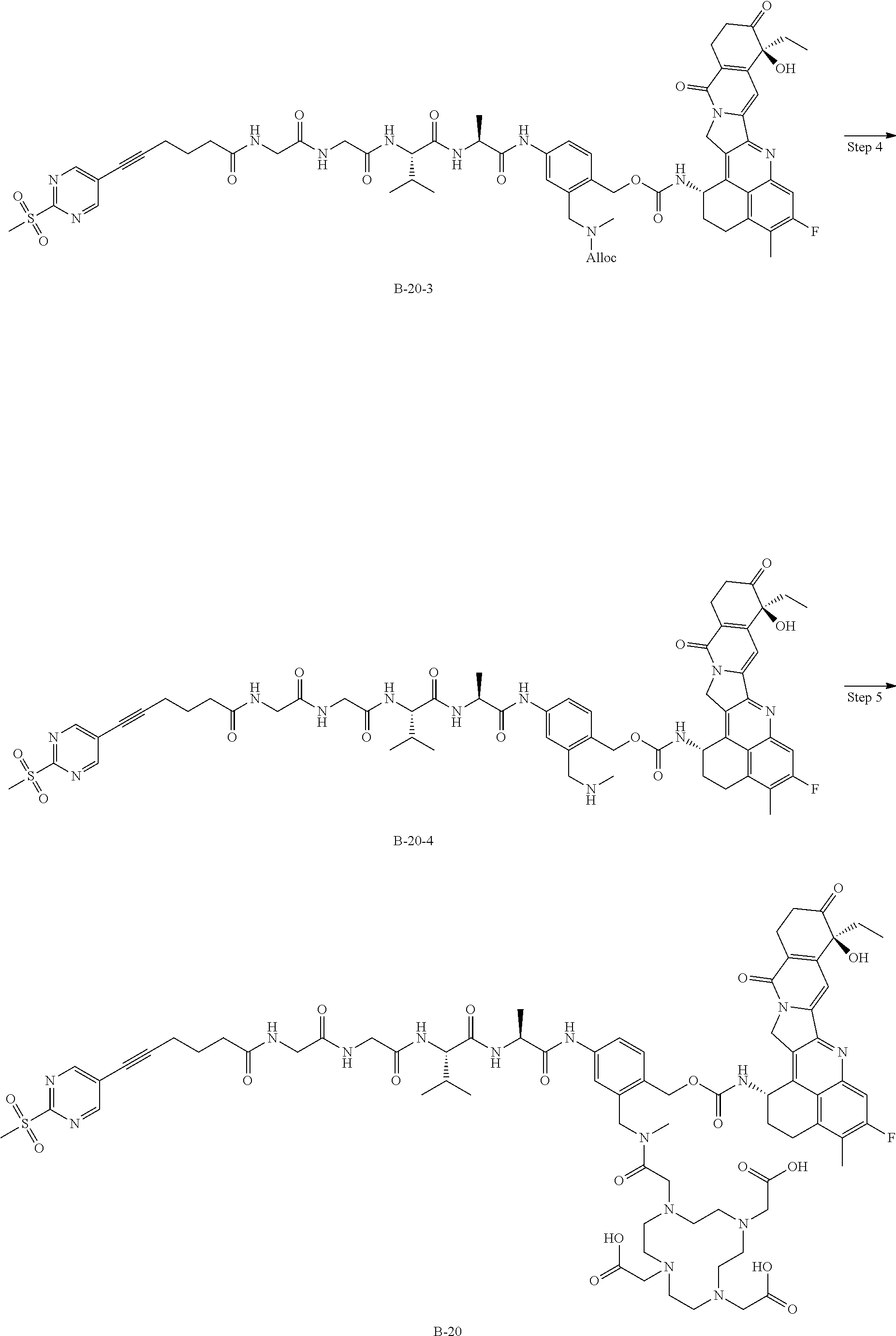

### Step 1: Preparation of allyl (5-((S)-2-((S)-2-((9H-fluoren-9-yl) methoxy)carbonyl)amino)-3-methylbutanamido) propionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) carbamoyl)oxy) methyl)benzyl) (methyl) carbamate (B-20-1)

Allyl (5((S)-2-aminopropionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (82.56 mg, 105.47 μmol) and (((9H-fluoren-9-yl)methoxy)carbonyl)-L-valine (35.89 mg, 105.47 μmol) were dissolved in DMF (1 mL), added with 2-(7-azobenzotriazole)-N,N,N′,N′-tetramethyluronium hexafluorophosphate (40.07 mg, 105.47 μmol) and DIPEA (40.82 mg, 316.41 μmol), and stirred at room temperature for 1 hour. After the reaction was completed, the reaction solution was purified by flash column chromatography (C18, water/acetonitrile=1/2) and freeze-dried to obtain the title compound (55 mg, 49.85 μmol).

Its structural characterization data are as follows:

MS m/z (ESI): 1104.4 [M+H]⁺

### Step 2: Preparation of allyl (5-((S)-2-aminopropionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (B-20-2)

Allyl (5-((S)-2-((S)-2-((9H-fluoren-9-yl)methoxy)carbonyl)amino)-3-methylbutanamido) propionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (55 mg, 49.85 μmol) was dissolved in DMF (1 mL), added with diethylamine (0.5 mL), and stirred at room temperature for 1 hour. After the reaction was completed, the reaction solution was purified by flash column chromatography (C18, water/acetonitrile=0.7) and freeze-dried to obtain the title compound (35.3 mg, 0.04 mmol).

Its structural characterization data are as follows:

MS m/z (ESI): 882.3 [M+H]⁺

### Step 3: Preparation of allyl (2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynyl)benzyl)(methyl)carbamate (B-20-3)

Allyl (5-((S)-2-aminopropionamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (35.3 mg, 0.04 mmol) and (6-(2-(methylsulfonyl)pyrimidin-5-yl)hex-5-ynoyl)glycylglycine (19.1 mg, 0.05 mmol) were dissolved in DMF (1 mL), added with 2-(7-azobenzotriazole)-N,N,N′,N′-tetramethyluronium hexafluorophosphate (19 mg, 0.05 mmol) and DIPEA (19.35 mg, 0.15 mol), and stirred at room temperature for 1 hour. After the reaction was completed, the reaction solution was purified by flash column chromatography (C18, water/acetonitrile=1/2) and freeze-dried to obtain the title compound (30 mg, 24.07 μmol).

Its structural characterization data are as follows:

MS m/z (ESI): 1246.4 [M+H]⁺

### Step 4: Preparation of 4-((2S,5S)-5-isopropyl-2-methyl-18-(2-(methylsulfonyl)pyrimidin-5-yl)-4,7,10,13-tetraoxo-3,6,9,12-tetraazaoctadec-17-ynamino)-2-((methylamino)methyl)benzyl((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (B-20-4)

Allyl (2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynyl)benzyl)(methyl)carbamate (30 mg, 24.07 μmol) was dissolved in DMF (1 mL), added with tetratriphenylphosphine palladium (2.1 mg, 2.41 μmol), then added with 15 μL of formic acid and 30 μL of N-methylmorpholine, and stirred at room temperature for 1 hour under nitrogen protection. After the reaction was completed, the reaction solution was purified by flash column chromatography (C18, water/acetonitrile=4/1) and freeze-dried to obtain the title compound (25 mg, 21.51 μmol).

Its structural characterization data are as follows:

MS m/z (ESI): 1162.2 [M+H]⁺

### Step 5: Preparation of 2,2′,2″-(10-(2-((2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)amino)-2-oxoethyl)-1,4,7,10-tetraazacyclododecan-1,4,7-triyl)triacetic acid (B-20)

4-((2S,5S)-5-Isopropyl-2-methyl-18-(2-(methylsulfonyl)pyrimidin-5-yl)-4,7,10,13-tetraoxo-3,6,9,12-tetraazaoctadec-17-ynylamino)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (25 mg, 21.51 μmol) was dissolved in DMF (1 mL), added with DIPEA (8.32 mg, 64.53 μmol), and added with 2,2′,2″-(10-(2-((2,5-dioxopyrrolidin-1-yl)oxy)-2-oxoethyl)-1,4,7,10-tetraazacyclododecan-1,4,7-triyl)triacetic acid (32.36 mg, 64.53 μmol). After the reaction was completed, the reaction solution was directly purified by preparative high performance liquid chromatography and freeze-dried to obtain the title compound (13.42 mg, 8.58 μmol).

Its structural characterization data are as follows:

MS m/z (ESI): 1549.6 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 32 |
| 4.00 | 10 | 90 | 32 |
| 24.00 | 90 | 10 | 32 |

### Example 59: Preparation of 2,2′,2″-(10-(2-((2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-5-((S)-2-((S)-3-methyl-2-(6-(2-(methylsulfonyl)pyrimidin-5-yl)hex-5-ynamido)butanamido)propionamido)benzyl)(methyl)amino)-2-oxoethyl)-1,4,7,10-tetraazacyclododecan-1,4,7-triyl)triacetic acid (B-23)

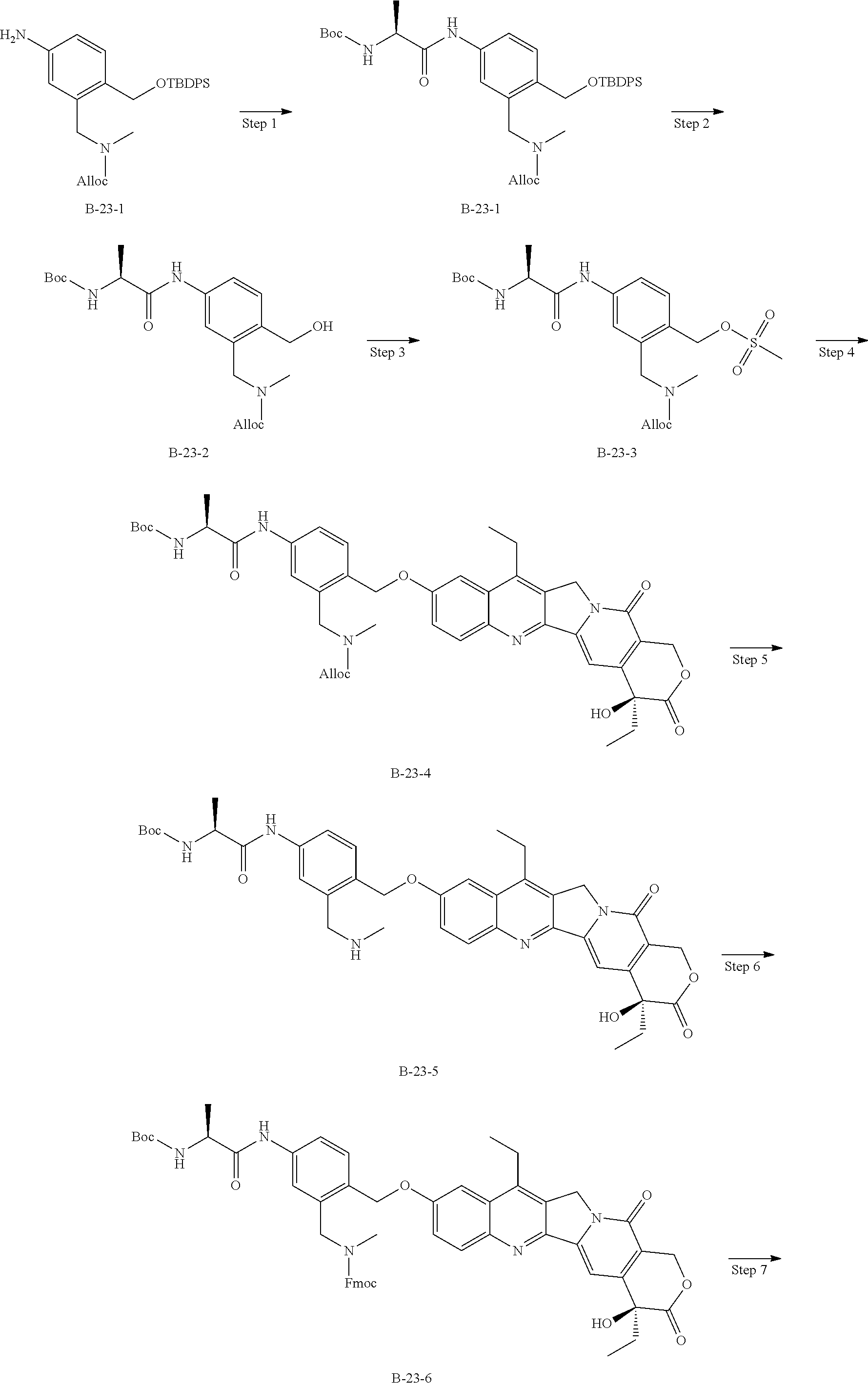

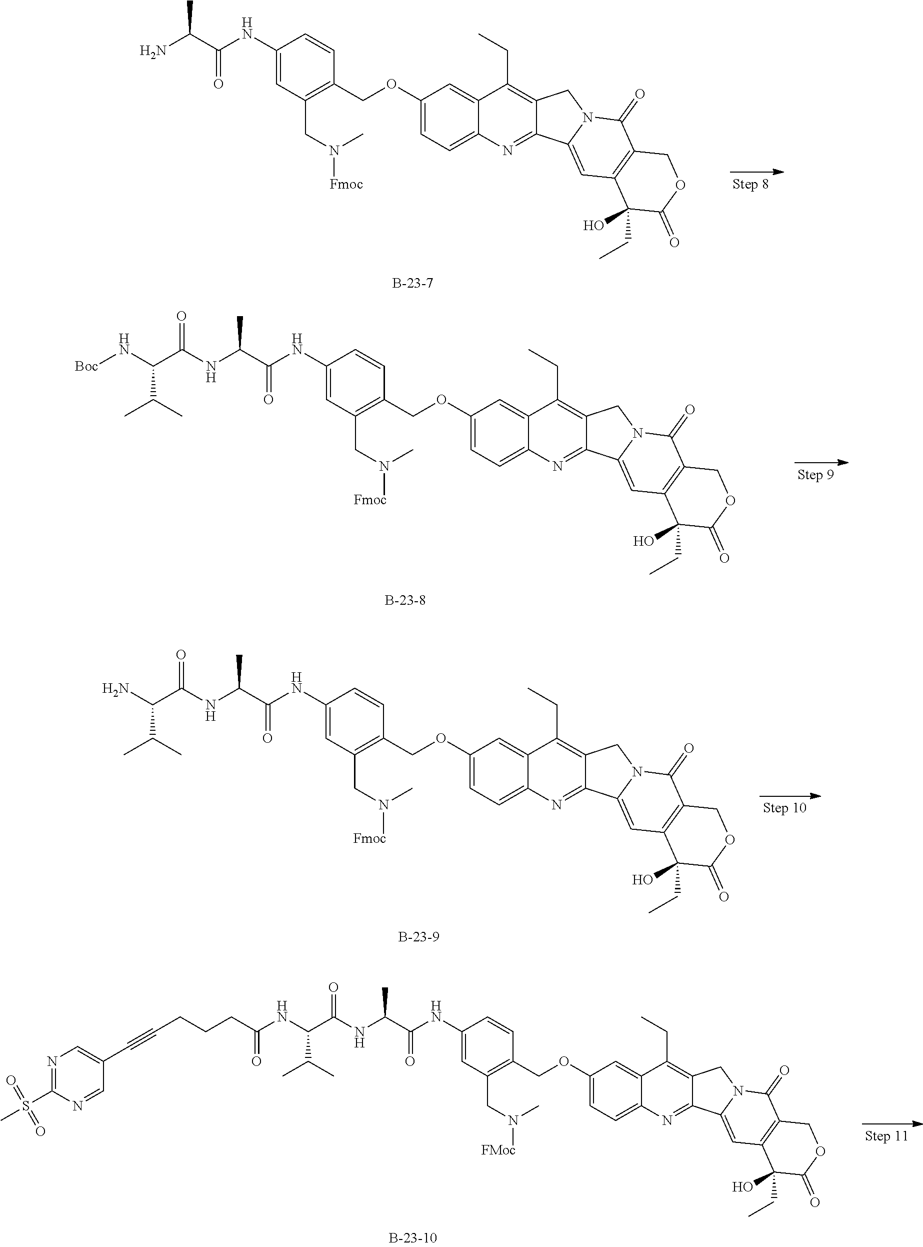

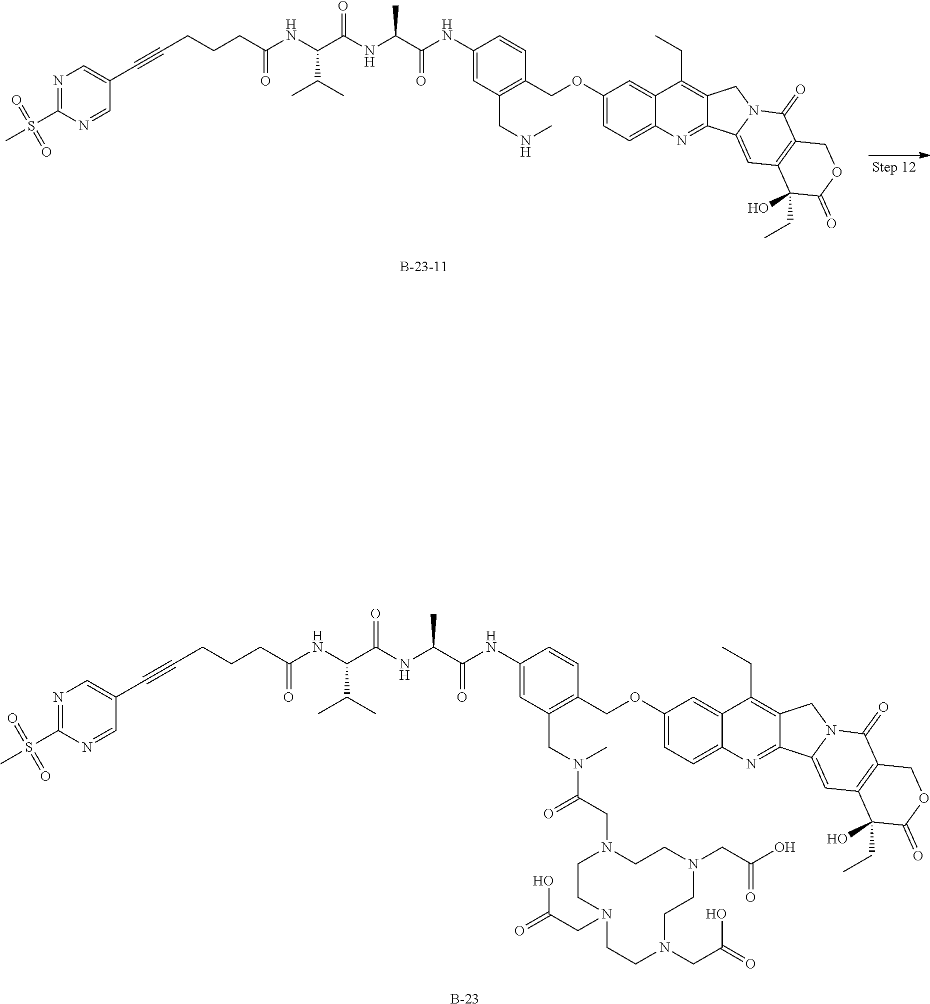

### Step 1. Preparation of allyl (S)-(5-(2-((tert-butyloxycarbonyl)amino)propionamido)-2-(((tert-butyldiphenylsilyl)oxy)methyl)benzyl)(methyl)carbamate (B-23-1)

Allyl (5-amino-2-(((tert-butyldiphenylsilyl)oxy)methyl)benzyl)(methyl)carbamate (5.04 g, 10.31 mmol) and (tert-butyloxycarbonyl)-L-alanine (1.95 g, 10.31 mmol) were dissolved in DCM (100 mL) and MeOH (25 mL), then added with 2-ethoxy-1-ethoxycarbonyl-1,2-dihydroquinoline (7.65 g, 30.92 mmol), and stirred at room temperature for 24 hours. The reaction solution was concentrated under reduced pressure to obtain a crude product, which was purified by silica gel column chromatography (ethyl acetate/petroleum ether=0 to 25%), and concentrated again under reduced pressure to obtain the title compound (5.95 g, 9.02 mmol).

Its structural characterization data are as follows:

MS m/z (ESI): 677.3 [M+H]⁺

### Step 2: Preparation of allyl (S)-(5-(2-((tert-butoxycarbonyl)amino)propionamido)-2-(hydroxymethyl)benzyl)(methyl)carbamate (B-23-2)

Allyl (S)-(5-(2-((tert-butoxycarbonyl)amino)propionamido)-2-(((tert-butyldiphenylsilyl)oxy) methyl)benzyl)(methyl)carbamate (5.95 g, 9.02 mmol) was dissolved in DMF (90 mL), added with pyridine hydrofluoride (13.40 g, 135.25 mmol), and stirred at 60° C. for 24 hours. After the reaction was completed, the reaction solution was added with water, extracted with ethyl acetate, dried over anhydrous sodium sulfate, and concentrated under reduced pressure to obtain the crude product of the title compound, which was purified by silica gel column chromatography (ethyl acetate/petroleum ether=0 to 70%), then concentrated again under reduced pressure to obtain the title compound (2.88 g, 6.83 mmol).

### Step 3: Preparation of benzyl (S)-2-((((allyloxy)carbonyl)(methyl)amino)methyl)-4-(2-((tert-butoxycarbonyl)amino)propionamido)methanesulfonate (B-23-3)

Allyl (S)-(5-(2-((tert-butoxycarbonyl)amino)propionamido)-2-(hydroxymethyl)benzyl) (methyl)carbamate (1.50 g, 3.56 mmol) was dissolved in DCM (150 mL), added with TEA (1.08 g, 10.68 mmol), and cooled in an ice-water bath for 15 minutes. Methanesulfonyl chloride (611.50 mg, 5.34 mmol) was dissolved in DCM (10 mL), and added dropwise to the reaction system. The reaction was carried out at 25° C. for 4 hours. After the reaction was completed, the reaction solution was added with water, extracted with DCM, dried over anhydrous sodium sulfate, and concentrated under reduced pressure to obtain the crude product of the title compound (1.39 g, 2.78 mmol).

### Step 4: Preparation of allyl (5-((S)-2-((tert-butoxycarbonyl)amino)propionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (B-23-4)

7-Ethyl-10-hydroxycamptothecin (615.00 mg, 1.57 mmol) and potassium carbonate (541.53 mg, 3.92 mmol) were dissolved in acetonitrile (50 mL), and stirred at room temperature for 10 minutes. Benzyl (S)-2-((((allyloxy)carbonyl)(methyl)amino)methyl)-4-(2-((tert-butoxycarbonyl) amino)propionamido)methanesulfonate (782.97 mg, 1.57 mmol) was dissolved in acetonitrile (50 mL), and added dropwise to the reaction system. The reaction was carried out at 65° C. for 1 hour. After the reaction was completed, the pH of the system was adjusted to about 2 with 1N HCl, stirred for 10 minutes, extracted with ethyl acetate, dried over anhydrous sodium sulfate, and concentrated under reduced pressure to obtain the crude product of the title compound (1.25 g, 1.57 mmol).

Its structural characterization data are as follows:

MS m/z (ESI): 796.3 [M+H]⁺

### Step 5: Preparation of tert-butyl ((S)-1-((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-3-((methylamino)methyl)phenyl)amino)-1-oxopropan-2-yl)carbamate (B-23-5)

Allyl (5-((S)-2-((tert-butoxycarbonyl)amino)propionamido)-2-((((S)-4,11-diethyl-4-hydroxy-3,14-dioxo-3,4,12,14-tetrahydro-TH-pyrano[3′,4′:6,7]indolizino[1,2-b]quinolin-9-yl)oxy)methyl)benzyl)(methyl)carbamate (750.00 mg, 942.36 μmol) was dissolved in methanol (150 mL), added with platinum dioxide (149.79 mg, 659.65 μmol), vacuumed, subjected to hydrogen replacement 3 times, and stirred at room temperature at 25° C. for 30 minutes. After the reaction was completed, the reaction solution was filtered with diatomaceous earth and concentrated to obtain the crude product of the title compound (670.00 mg, 941.27 μmol).

Its structural characterization data are as follows:

MS m/z (ESI): 712.5 [M+H]⁺

### Step 6: Preparation of (9H-fluoren-9-yl)methyl (5-((S)-2-((tert-butyloxycarbonyl)amino) propionamido)-2-((((S)-4,11-diethyl-4-hydroxy-3,14-dioxo-3,4,12,14-tetrahydro-TH-pyrano[3′,4′:6,7]indolizino[1,2-b]quinolin-9-yl)oxy)methyl)benzyl)(methyl)carbamate (B-23-6)

Tert-butyl ((S)-1-((4-((((S)-4,11-diethyl-4-hydroxy-3,14-dioxo-3,4,12,14-tetrahydro-H-pyrano[3′,4′:6,7]indolizino[1,2-b]quinolin-9-yl)oxy)methyl)-3-((methylamino)methyl)phenyl)amino)-1-oxopropan-2-yl)carbamate (1.12 g, 1.57 mmol) was dissolved in 1,4-dioxane (75 mL) and water (75 mL), and added with sodium bicarbonate (2.64 g, 31.47 mmol) and 9-fluorenylmethyl-N-succinimidyl carbonate (424.62 mg, 1.26 mmol). The reaction was carried out under stirring at room temperature for 2 hours. After the reaction was completed, the pH of the system was adjusted to about 3 with 1N HCl, extracted with ethyl acetate, and concentrated to obtain a crude product, which was purified by flash column chromatography (ACN/H₂O=0-65%, 0.05% ammonium bicarbonate) and freeze-dried to obtain the title compound (694.00 mg, 743.01 μmol).

Its structural characterization data are as follows:

MS m/z (ESI): 935.5 [M+H]⁺

### Step 7: Preparation of (9H-fluoren-9-yl)methyl (5-((S)-2-aminopropionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (B-23-7)

(9H-Fluoren-9-yl)methyl (5-((S)-2-((tert-butoxycarbonyl)amino)propionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (100.00 mg, 107.06 μmol) was dissolved in DCM (6 mL) and TFA (3 mL), and stirred at room temperature for 1.5 hours. After the reaction was completed, the reaction solution was concentrated to obtain the crude product of the title compound (101.00 mg, 106.55 μmol).

Its structural characterization data are as follows:

MS m/z (ESI): 835.3 [M+H]⁺

### Step 8: Preparation of (9H-fluoren-9-yl)methyl (5-((S)-2-((S)-2-((tert-butoxycarbonyl) amino)-3-methylbutanamido)propionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (B-23-8)

(9H-Fluoren-9-yl)methyl (5-((S)-2-aminopropionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (75.00 mg, 79.12 μmol) and DIPEA (51.13 mg, 395.59 μmol) were added to DMF (10 mL), stirred for 10 minutes, then added with (tert-butyloxycarbonyl)-L-valine (34.38 mg, 158.24 μmol) and DMTMM (46.64 mg, 158.24 μmol), and stirred at 25° C. for 2 hours. After the reaction was completed, the reaction solution was directly purified by preparative high performance liquid chromatography and freeze-dried to obtain the title compound (49.00 mg, 47.43 μmol).

Its structural characterization data are as follows:

MS m/z (ESI): 1033.6 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 50 | 90 | 30 |
| 2.00 | 50 | 90 | 30 |
| 18.00 | 90 | 10 | 30 |

### Step 9: Preparation of (9H-fluoren-9-yl)methyl (5-((S)-2-aminopropionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (B-23-9)

(9H-Fluoren-9-yl)methyl (5-((S)-2-((S)-2-((tert-butyloxycarbonyl)amino)-3-methyl-butanamido)propionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (68.00 mg, 65.82 μmol) was dissolved in DCM (6 mL) and TFA (3 mL), stirred at room temperature for 1 hour. After the reaction was completed, the reaction solution was concentrated to obtain the crude product of the title compound (68.00 mg, 64.94 μmol).

Its structural characterization data are as follows:

MS m/z (ESI): 933.5 [M+H]⁺

### Step 10: Preparation of (9H-fluoren-9-yl)methyl (2-((((S)-4,11-diethyl-4-hydroxy-3,14-dioxo-3,4,12,14-tetrahydro-1H-pyran)[3′,4′:6,7]indolizino[1,2-b]quinolin-9-yl)oxy)methyl)-5-((S)-2-((S)-3-methyl-2-(6-(2-(methylsulfonyl)pyrimidin-5-yl)hex-5-ynamido)butanamido)propionamido)benzyl)(methyl)carbamate (B-23-10)

(9H-Fluoren-9-yl)methyl (5-((S)-2-aminopropionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (68.00 mg, 64.94 μmol) and 2,5-dioxopyrrolidin-1-yl 6-(2-(methylsulfonyl)pyrimidin-5-yl)hex-5-ynoate (29.66 mg, 81.18 μmol) were dissolved in DMF (8 mL), and stirred at room temperature for 3 hours. After the reaction was completed, the reaction solution was directly purified by preparative high performance liquid chromatography and freeze-dried to obtain the title compound (42.00 mg, 35.49 μmol).

Its structural characterization data are as follows:

MS m/z (ESI): 1183.6 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 35 | 65 | 30 |
| 4.00 | 35 | 65 | 30 |
| 24.00 | 90 | 10 | 30 |

### Step 11: Preparation of N—((S)-1-(((S)-1-((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-3-((methylamino)methyl)phenyl)amino)-1-oxopropan-2-yl)amino)-3-methyl-1-oxobutan-2-yl)-6-(2-(methylsulfonyl)pyrimidin-5-yl)hex-5-ynamide (B-23-11)

(9H-Fluoren-9-yl)methyl (2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-5-((S)-2-((S)-3-methyl-2-(6-(2-(methylsulfonyl)pyrimidin-5-yl)hexan-5-amido)butanamido)propionamido)benzyl)(methyl)carbamate (27.00 mg, 22.82 μmol) and DBU (10.42 mg, 68.45 μmol) were dissolved in DMF (3 mL), and stirred at room temperature for 30 minutes. After the reaction was completed, a crude product (21 mg, 21.85 μmol) was obtained, which was not purified and directly used in the next step.

Its structural characterization data are as follows:

MS m/z (ESI): 961.4 [M+H]⁺

### Step 12: Preparation of 2,2′,2″-(10-(2-((2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-5-((S)-2-((S)-3-methyl-2-(6-(2-(methylsulfonyl)pyrimidin-5-yl)hex-5-ynamido)butanamido)propionamido)benzyl)(methyl)amino)-2-oxoethyl)-1,4,7,10-tetracyclododecan-1,4,7-triyl)triacetic acid (B-23)

N—((S)-1-(((S)-1-((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-3-((methylamino)methyl)phenyl)amino)-1-oxopropan-2-yl)amino)-3-methyl-1-oxobutan-2-yl)-6-(2-(methylsulfonyl)pyrimidin-5-yl)hex-5-ynamide (21 mg, 21.85 μmol) was dissolved in DMF (3 mL), added with DIPEA (33.89 mg, 262.20 μmol) and 2,2′,2″-(10-(2-((2,5-dioxopyrrolidin-1-yl)oxy)-2-oxoethyl)-1,4,7,10-tetraazacyclododecan-1,4,7-triyl)triacetic acid (54.79 mg, 109.25 μmol), and stirred at 60° C. for 1.5 hours. After the reaction was completed, the reaction solution was directly purified by preparative high performance liquid chromatography, adjusted to pH of about 2 with TFA, and freeze-dried to obtain the title compound (11.50 mg, 7.48 μmol).

Its structural characterization data are as follows:

MS m/z (ESI): 1347.7 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 15 | 85 | 30 |
| 4.00 | 15 | 85 | 30 |
| 24.00 | 90 | 10 | 30 |

### Example 60: Preparation of 2,2′,2″-(10-(2-((2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)amino)-2-oxoethyl)-1,4,7,10-tetraazacyclododecan-1,4,7-triyl)triacetic acid (B-22)

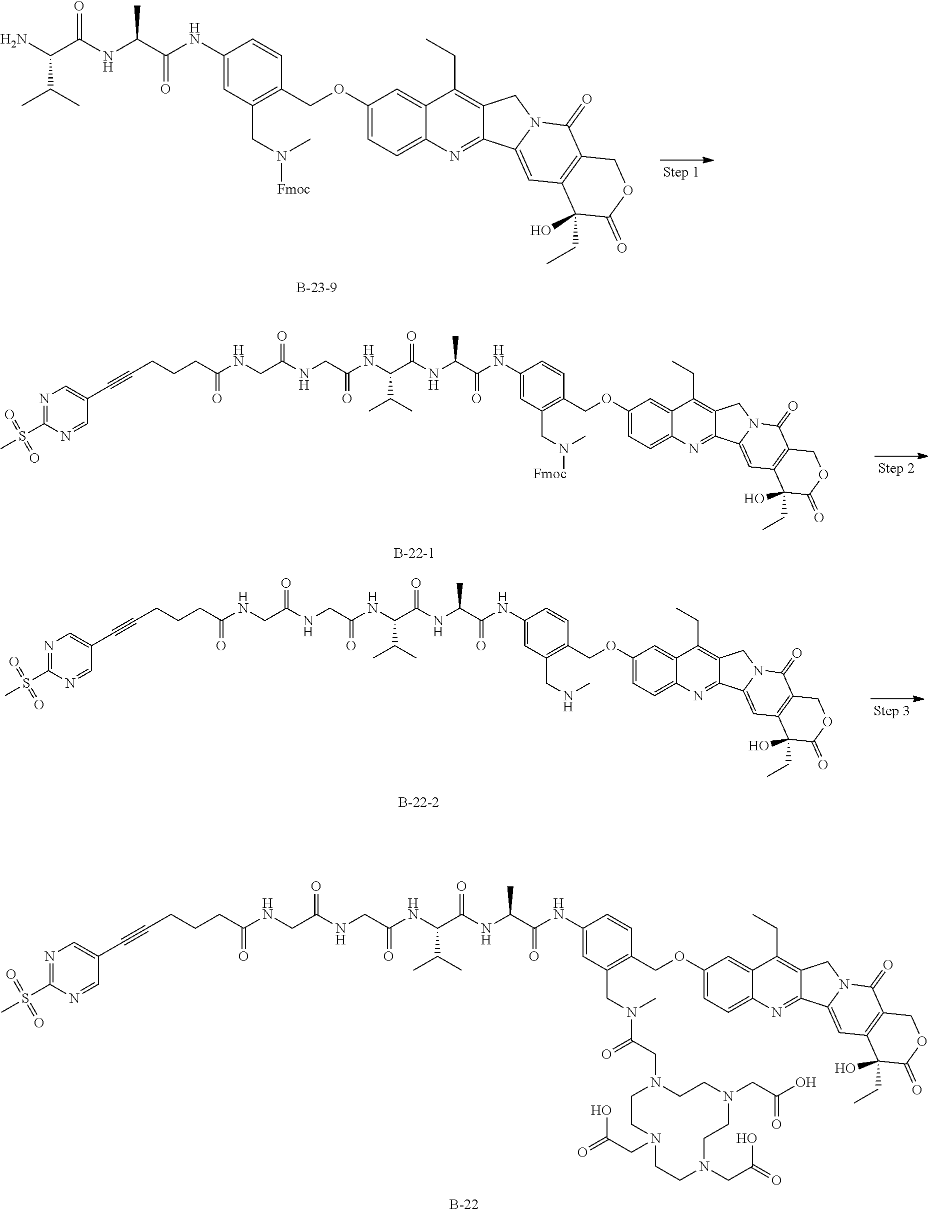

### Step 1: Preparation of (9H-fluoren-9-yl)methyl (2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)carbamate (B-22-1)

(9H-Fluoren-9-yl)methyl (5-((S)-2-aminopropionamido)-2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)benzyl)(methyl)carbamate (91 mg, 86.91 μmol) and DIPEA (112.32 mg, 869.08 μmol) were dissolved in DMF (10 mL), stirred for 10 minutes, then added with (6-(2-(methylsulfonyl)pyrimidin-5-yl)hex-5-ynoyl)glycylglycine (INT-3) (39.88 mg, 104.29 μmol) and DMTMM (51.23 mg, 173.82 μmol), and stirred at 25° C. for 3 hours. After the reaction was completed, the reaction solution was directly purified by preparative high performance liquid chromatography and freeze-dried to obtain the title compound (72.00 mg, 55.49 μmol).

Its structural characterization data are as follows:

MS m/z (ESI): 1297.5 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 40 | 60 | 30 |
| 2.00 | 40 | 60 | 30 |
| 20.00 | 90 | 10 | 30 |

### Step 2: Preparation of N-(2-((2-(((S)-1-(((S)-1-((4-(((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-3-((methylamino)methyl)phenyl)amino)-1-oxopropan-2-yl)amino)-3-methyl-1-oxobutan-2-yl)amino)-2-oxoethyl)amino)-2-oxoethyl)-6-(2-(methylsulfonyl)pyrimidin-5-yl)hex-5-ynamide (B-22-2)

(9H-Fluoren-9-yl)methyl (2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)carbamate (25.00 mg, 19.27 μmol) and DBU (8.80 mg, 57.81 μmol) were dissolved in DMF (4 mL), and stirred at room temperature for 1 hour. After the reaction was completed, the crude product (20.00 mg, 18.60 μmol) was obtained and used directly in the next step without purification.

Its structural characterization data are as follows:

MS m/z (ESI): 1075.6 [M+H]⁺

### Step 3: Preparation of 2,2′,2″-(10-(2-((2-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)amino)-2-oxoethyl)-1,4,7,10-tetraazacyclododecan-1,4,7-triyl)triacetic acid (B-22)

N-(2-((2-(((S)-1-(((S)-1-((4-((((S)-4,11-diethyl-4-hydroxy-3,14-dioxo-3,4,12,14-tetrahydro-1H-pyrano[3′,4′:6,7]indolizino[1,2-b]quinolin-9-yl)oxy)methyl)-3-((methylamino)methyl)phenyl)amino)-1-oxopropan-2-yl)amino)-3-methyl-1-oxobutan-2-yl)amino)-2-oxoethyl)amino)-2-oxoethyl)-6-(2-(methylsulfonyl)pyrimidin-5-yl)hex-5-ynamide (20.00 mg, 18.60 μmol) was dissolved in DMF (4 mL), added with DIPEA (28.85 mg, 223.22 μmol) and 2,2′,2″-(10-(2-((2,5-dioxopyrrolidin-1-yl)oxy)-2-oxoethyl)-1,4,7,10-tetraazacyclododecan-1,4,7-triyl)triacetic acid (46.64 mg, 93.01 μmol), and reacted under stirring at 60° C. for 1.5 hours. After the reaction was completed, the reaction solution was directly purified by preparative high performance liquid chromatography, adjusted to the pH of about 2 with TFA, and freeze-dried to obtain the title compound (6.00 mg, 3.58 μmol).

Its structural characterization data are as follows:

MS m/z (ESI): 1462.7 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 30 |
| 2.00 | 10 | 90 | 30 |
| 18.00 | 90 | 10 | 30 |

### Example 61: Preparation of 2,2′,2″-(10-(2-((2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-5-((S)-2-((S)-3-methyl-2-(6-(2-(methylsulfonyl)pyrimidin-5-yl)hex-5-ynamido)butanamido)propionamido)benzyl)(methyl)amino)-2-oxoethyl)-1,4,7,10-tetraazacyclododecan-1,4,7-triyl)triacetic acid (A-187)

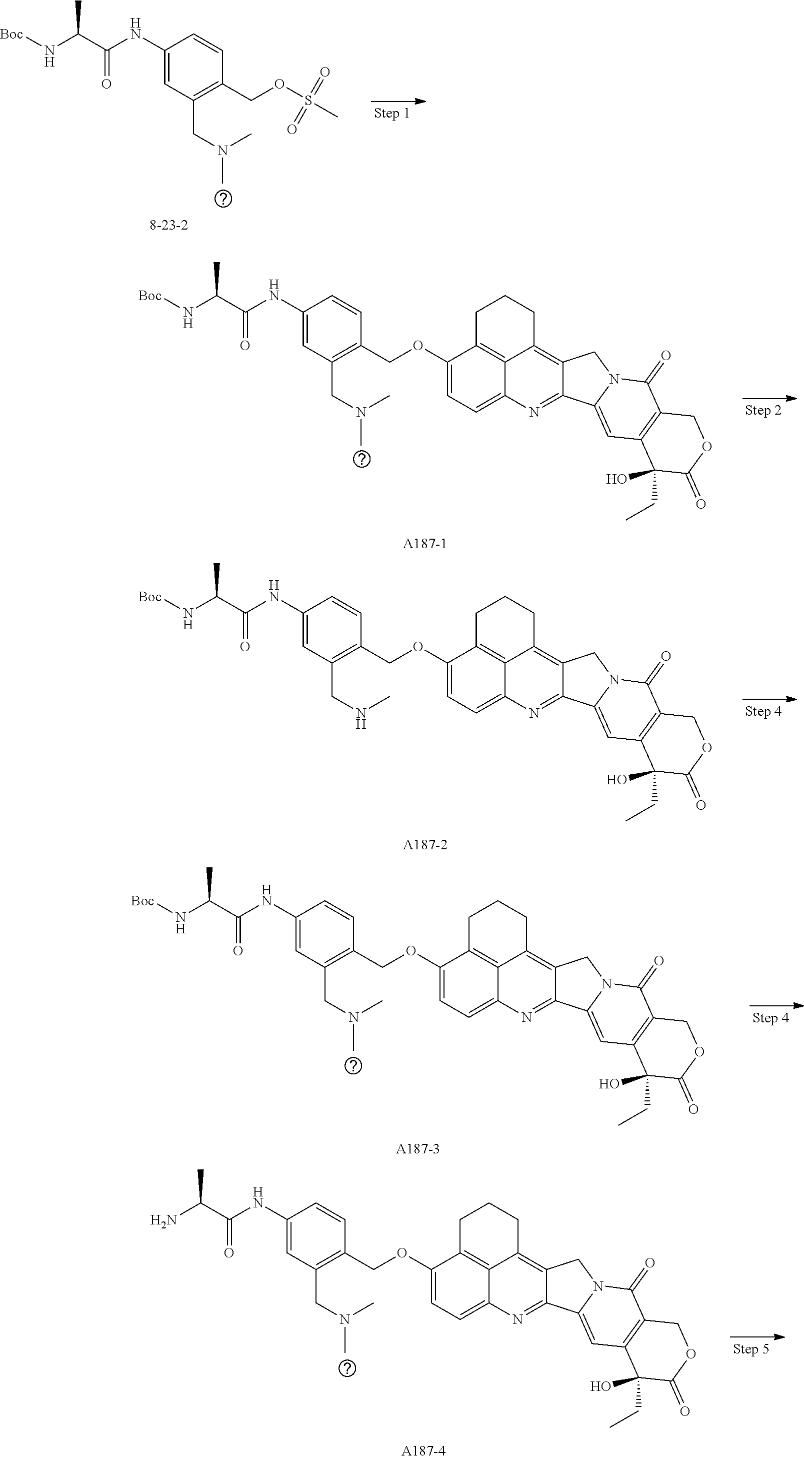 [SMILES: C[C@H](NC(=O)OC(C)(C)C)C(=O)Nc1ccc(COS(C)(=O)=O)c(CN(C)C)c1 ; CC[C@@]1(O)C(=O)OCc2c1cc1n(c2=O)Cc2c-1nc1ccc(OCc3ccc(NC(=O)[C@H](C)NC(=O)OC(C)(C)C)cc3CN(C)C)c3c1c2CCC3 ; CC[C@@]1(O)C(=O)OCc2c1cc1n(c2=O)Cc2c-1nc1ccc(OCc3ccc(NC(=O)[C@H](C)NC(=O)OC(C)(C)C)cc3CNC)c3c1c2CCC3 ; CC[C@@]1(O)C(=O)OCc2c1cc1n(c2=O)Cc2c-1nc1ccc(OCc3ccc(NC(=O)[C@H](C)NC(=O)OC(C)(C)C)cc3CN(C)C)c3c1c2CCC3 ; CC[C@@]1(O)C(=O)OCc2c1cc1n(c2=O)Cc2c-1nc1ccc(OCc3ccc(NC(=O)[C@H](C)N)cc3CN(C)C)c3c1c2CCC3]

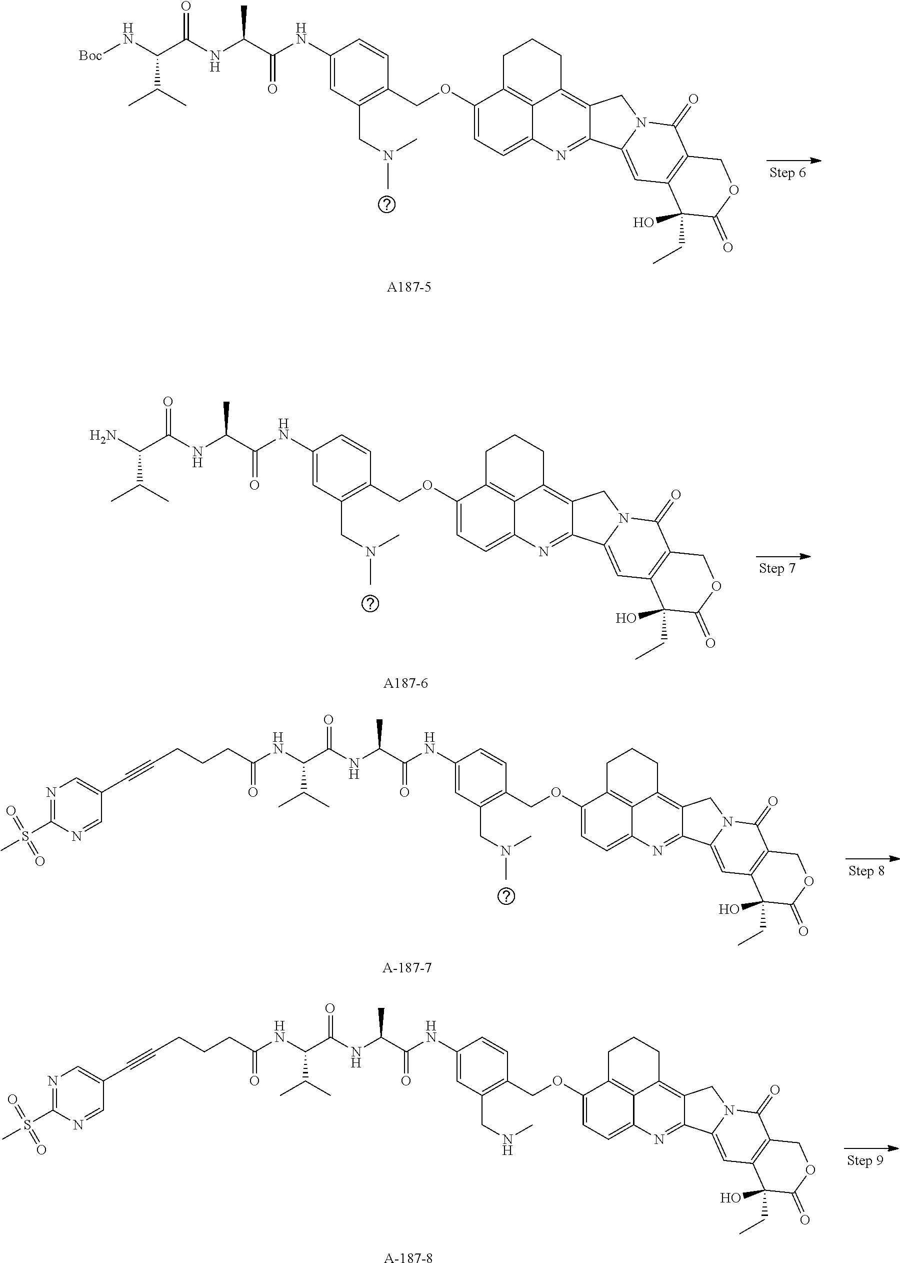

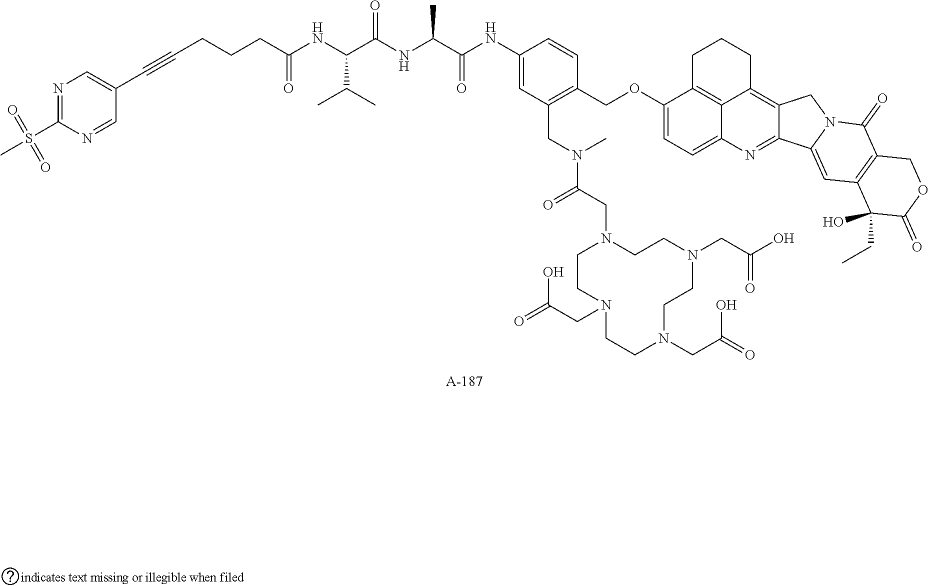 [SMILES: CC[C@@]1(O)C(=O)OCc2c1cc1n(c2=O)Cc2c-1nc1ccc(OCc3ccc(NC(=O)[C@H](C)NC(=O)[C@@H](NC(=O)CCCC#Cc4cnc(S(C)(=O)=O)nc4)C(C)C)cc3CN(C)C(=O)CN3CCN(CC(=O)O)CCN(CC(=O)O)CCN(CC(=O)O)CC3)c3c1c2CCC3 ; "A-187"]

Step 1: Preparation of allyl (5-((S)-2-((tert-butoxycarbonyl)amino) propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (A-187-1)

(S)-9-Ethyl-4,9-dihydroxy-1,2,3,9,12,15-hexahydro-10H,13H-benzo[de]pyrano[3′,4′:6,7]indolizino[1,2-b]quinolin-10,13-dione (1.13 g, 2.78 mmol) and potassium carbonate (961.37 mg, 6.96 mmol) were dissolved in acetonitrile (100 mL), and stirred at room temperature for 10 minutes. Benzyl (S)-2-((((allyloxy)carbonyl)(methyl)amino)methyl)-4-(2-((tert-butoxycarbonyl) amino)propionamido)methanesulfonate (1.13 g, 2.78 mmol) was dissolved in acetonitrile (100 mL), then added dropwise to the reaction system, and reacted at 65° C. for 1 hour. After the reaction was completed, the system was adjusted to pH of about 2 with 1N HCl, stirred for 15 minutes, extracted with ethyl acetate, dried over anhydrous sodium sulfate, and concentrated under reduced pressure to obtain the crude product of the title compound (2.23 g, 2.76 mmol).

Its structural characterization data are as follows:

MS m/z (ESI): 808.3 [M+H]⁺

### Step 2: Preparation of tert-butyl ((S)-1-((4-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-3-((methylamino)methyl)phenyl)amino)-1-oxopropan-2-yl)carbamate (A-187-2)

Allyl (5-((S)-2-((tert-butoxycarbonyl)amino)propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (1.38 g, 1.71 mmol) was dissolved in methanol (280 mL), added with platinum dioxide (193.94 mg, 854.08 μmol), vacuumed, then subjected to hydrogen replacement three times, and stirred at room temperature at 25° C. for 30 minutes. After the reaction was completed, the reaction solution was filtered through diatomaceous earth and concentrated to obtain the crude product of the title compound (1.24 g, 1.71 mmol).

Its structural characterization data are as follows:

MS m/z (ESI): 724.6 [M+H]⁺

### Step 3: Preparation of (9H-fluoren-9-yl)methyl (5-((S)-2-((tert-butyloxycarbonyl)amino) propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (A-187-3)

Tert-butyl (S)-1-((4-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-3-((methylamino)methyl)phenyl)amino)-1-oxopropan-2-yl)carbamate (1.25 g, 1.73 mmol) was dissolved in 1,4-dioxane (100 mL) and water (100 mL), added with sodium bicarbonate (2.90 g, 34.54 mmol) and Fmoc-OSU (active ester) (407.79 mg, 1.21 mmol). The reaction was carried out under stirring at room temperature for 2 hours. After the reaction was completed, the system was adjusted to pH of about 3 with 1N dilute hydrochloric acid, extracted with ethyl acetate, and concentrated to obtain a crude product, which was purified by preparative high performance liquid chromatography, and freeze-dried to obtain the title compound (104 mg, 109.93 μmol).

Its structural characterization data are as follows:

MS m/z (ESI): 946.5 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 55 | 45 | 30 |
| 2.00 | 55 | 45 | 30 |
| 24.00 | 90 | 10 | 30 |

### Step 4: Preparation of (9H-fluoren-9-yl)methyl (5-((S)-2-aminopropionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (A-187-4)

(9H-fluoren-9-yl)methyl (5-((S)-2-((tert-butoxycarbonyl)amino)propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (140 mg, 147.98 μmol) was dissolved in DCM (6 mL) and TFA (3 mL), and stirred at room temperature for 1.5 hours. After the reaction was completed, the reaction solution was concentrated to obtain the crude product of the title compound (141.00 mg, 146.88 μmol).

### Step 5: Preparation of (9H-fluoren-9-yl)methyl (5-((S)-2-((S)-2-((tert-butoxycarbonyl) amino)-3-methylbutanamido)propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (A-187-5)

(9H-Fluoren-9-yl)methyl (5-((S)-2-aminopropionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (141.00 mg, 146.88 μmol) and DIPEA (94.91 mg, 734.41 μmol) were added to DMF (8 mL), stirred for 10 min, then added with (tert-butyloxycarbonyl)-L-valine (63.82 mg, 293.76 μmol) and DMTMM (86.58 mg, 293.76 μmol), and stirred at 25° C. for 2 hours. After the reaction was completed, the reaction solution was directly purified by preparative high performance liquid chromatography and freeze-dried to obtain the title compound (102.00 mg, 97.59 μmol).

Its structural characterization data are as follows:

MS m/z (ESI): 1045.6 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 50 | 90 | 30 |
| 2.00 | 50 | 90 | 30 |
| 18.00 | 95 | 5 | 30 |

### Step 6: Preparation of (9H-fluoren-9-yl)methyl (5-((S)-2-((S)-2-amino-3-methylbutanamido) propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (A-187-6)

(9H-Fluoren-9-yl)methyl (5-((S)-2-((S)-2-((tert-butoxycarbonyl)amino)-3-methylbutanamido)propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (40.00 mg, 38.27 μmol) was dissolved in DCM (3 mL) and TFA (1.5 mL), and stirred at room temperature for 1.5 hours. After the reaction was completed, the reaction solution was concentrated to obtain the crude product of the title compound (40.00 mg, 40.53 μmol).

Its structural characterization data are as follows:

MS m/z (ESI): 945.6 [M+H]⁺

### Step 7: Preparation of (9H-fluoren-9-yl)methyl (2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-5-((S)-2-((S)-3-methyl-2-(6-(2-(methylsulfonyl)pyrimidin-5-yl)hex-5-ynamido)butanamido)propionamido)benzyl)(methyl)carbamate (A-187-7)

(9H-Fluoren-9-yl)methyl (5-((S)-2-((S)-2-amino-3-methylbutanamido)propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (40.00 mg, 40.53 μmol) and 6-(2-(methylsulfonyl)pyrimidin-5-yl)hex-5-ynoic acid (15.20 mg, 56.65 μmol) were dissolved in DMF (4 mL), then added with DIPEA (29.29 mg, 226.61 μmol) and DMTMM (22.26 mg, 75.54 μmol), and stirred at room temperature for 3 hours. After the reaction was completed, the reaction solution was directly purified by preparative high performance liquid chromatography and freeze-dried to obtain the title compound (28.00 mg, 23.42 μmol).

Its structural characterization data are as follows:

MS m/z (ESI): 1195.6 [M+H]⁺

Its preparation method was as follows:

Chromatographic column: Waters Xbridge Prep
C18 OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 40 | 60 | 30 |
| 2.00 | 40 | 60 | 30 |
| 18.00 | 95 | 5 | 30 |

### Step 8: Preparation of N—((S)-1-(((S)-1-((4-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-3-((methylamino)methyl)phenyl)amino)-1-oxopropan-2-yl)amino)-3-methyl-1-oxobutan-2-yl)-6-(2-(methylsulfonyl)pyrimidin-5-yl)hex-5-ynamide (A-187-8)

(9H-Fluoren-9-yl)methyl (2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-5-((S)-2-((S)-3-methyl-2-(6-(2-(methylsulfonyl)pyrimidin-5-yl)hex-5-ynamido)butanamido)propionamido)benzyl)(methyl)carbamate (28.00 mg, 23.42 μmol) and DBU (10.70 mg, 70.27 μmol) were dissolved in DMF (4 mL), and stirred at room temperature for 30 minutes. After the reaction was completed, the crude product (22.00 mg, 22.61 μmol) was obtained and used directly in the next step without purification.

Its structural characterization data are as follows:

MS m/z (ESI): 973.5 [M+H]⁺

### Step 9: Preparation of 2,2′,2″-(10-(2-((2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-5-((S)-2-((S)-3-methyl-2-(6-(2-(methylsulfonyl)pyrimidin-5-yl)hexan-5-amido)butanamido)propionamido)benzyl)(methyl)amino)-2-oxoethyl)-1,4,7,10-tetraazacyclododecan-1,4,7-triyl)triacetic acid (A-187)

N—((S)-1-(((S)-1-((4-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-3-((methylamino)methyl)phenyl)amino)-1-oxopropan-2-yl)amino)-3-methyl-1-oxobutan-2-yl)-6-(2-(methylsulfonyl)pyrimidin-5-yl)hex-5-ynamide (22 mg, 22.61 μmol) was dissolved in DMF (4 mL), added with DIPEA (35.06 mg, 271.30 μmol) and 2,2′,2″-(10-(2-((2,5-dioxopyrrolidin-1-yl)oxy)-2-oxoethyl)-1,4,7,10-tetraazacyclododecan-1,4,7-triyl)triacetic acid (56.69 mg, 113.04 μmol), and stirred at 60° C. for 1.5 hours. After the reaction was completed, the reaction solution was directly purified by preparative high performance liquid chromatography, then adjusted with TFA to have a system pH of about 2, and freeze-dried to obtain the title compound (13.00 mg, 8.38 μmol).

Its structural characterization data are as follows:

MS m/z (ESI): 1360.6 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 30 |
| 2.00 | 10 | 90 | 30 |
| 18.00 | 80 | 20 | 30 |

### Example 62: Preparation of 2,2′,2″-(10-(2-((2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)amino)-2-oxoethyl)-1,4,7,10-tetraazacyclododecan-1,4,7-triyl)triacetic acid (B-21)

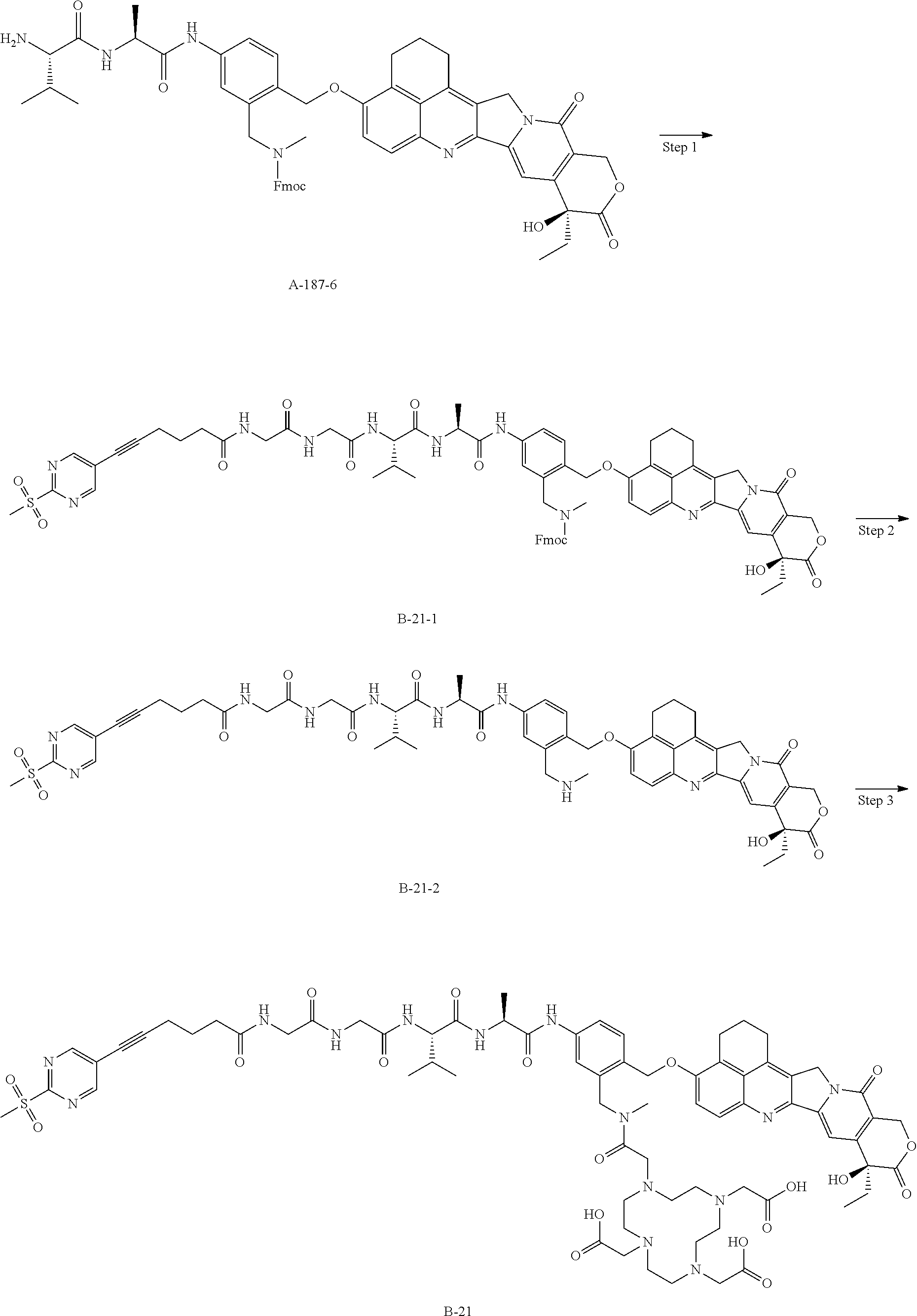

### Step 1: Preparation of (9H-fluoren-9-yl)methyl (2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)carbamate (B-21-1)

(9H-fluoren-9-yl)methyl (5-((S)-2-((S)-2-amino-3-methylbutanamido)propionamido)-2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)benzyl)(methyl)carbamate (70.00 mg, 70.07 μmol) and DIPEA (95.73 mg, 740.69 μmol) were dissolved in DMF (6 mL), stirred for 10 minutes, then added with (6-(2-(methylsulfonyl)pyrimidin-5-yl)hex-5-ynoyl)glycylglycine (INT-3) (33.99 mg, 88.88 μmol) and DMTMM (43.66 mg, 148.14 μmol), and stirred at 25° C. for 3 hours. After the reaction was completed, the reaction solution was directly purified by preparative high performance liquid chromatography and freeze-dried to obtain the title compound (71.00 mg, 54.22 μmol).

Its structural characterization data are as follows:

MS m/z (ESI): 1310.6 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 40 | 60 | 30 |
| 4.00 | 40 | 60 | 30 |
| 24.00 | 90 | 10 | 30 |

### Step 2: Preparation of N-(2-((2-(((S)-1-(((S)-1-((4-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-3-((methylamino)methyl)phenyl)amino)-1-oxopropan-2-yl)amino)-3-methyl-1-oxobutan-2-yl)amino)-2-oxoethyl)amino)-2-oxoethyl)-6-(2-(methylsulfonyl)pyrimidin-5-yl)hex-5-ynamide (B-21-2)

(9H-Fluoren-9-yl)methyl (2-((((S)-9-ethyl-9-hydroxy-10,13-dioxo-2,3,9,10,13,15-hexahydro-1H,12H-benzo[de]pyrano[3′,4′:6,7]indolizino [1,2-b]quinolin-4-yl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)carbamate (40.00 mg, 30.55 μmol) and DBU (13.95 mg, 91.64 μmol) were dissolved in DMF (4 mL), and stirred at room temperature for 1 hour. After the reaction, the crude product (33.00 mg, 30.35 μmol) was obtained, and used directly in the next step without purification.

Its structural characterization data are as follows:

MS m/z (ESI): 1087.6 [M+H]⁺

### Step 3: Preparation of 2,2′,2″-(10-(2-((2-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-5-((2S,5S)-5-isopropyl-2-methyl-18-(2-(methylsulfonyl)pyrimidin-5-yl)-4,7,10,13-tetraoxo-3,6,9,12-tetraazaoctadec-17-ynamido)benzyl)(methyl)amino)-2-oxoethyl)-1,4,7,10-tetraazacyclododecan-1,4,7-triyl)triacetic acid (B-21)

N-(2-((2-(((S)-1-(((S)-1-((4-((((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)oxy)methyl)-3-((methylamino)methyl)phenyl)amino)-1-oxopropan-2-yl)amino)-3-methyl-1-oxobutan-2-yl)amino)-2-oxoethyl)amino)-2-oxoethyl)-6-(2-(methylsulfonyl)pyrimidin-5-yl)hex-5-ynamide (33.00 mg, 30.35 μmol) was dissolved in DMF (4 mL), added with DIPEA (47.07 mg, 364.24 μmol) and 2,2′,2″-(10-(2-((2,5-dioxopyrrolidin-1-yl)oxy)-2-oxoethyl)-1,4,7,10-tetraazacyclododecan-1,4,7-triyl)triacetic acid (76.11 mg, 151.77 μmol), stirred at 60° C. for 1.5 hours. After the reaction was completed, the reaction solution was directly purified by preparative high performance liquid chromatography, adjusted with TFA to have a system pH of about 2, and freeze-dried to obtain the title compound (25.00 mg, 14.96 μmol).

Its structural characterization data are as follows:

MS m/z (ESI): 1474.7 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 30 |
| 2.00 | 10 | 90 | 30 |
| 18.00 | 80 | 20 | 30 |

### Example 63: Preparation of 2,2′,2″-(10-((4R,9S,12S,15S,18S)-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-9,12,15-trimethyl-18-(6-(2-(methylsulfonyl)pyrimidin-5-yl)hex-5-ynamido)-3,8,11,14,17,24-hexaoxo-5-oxa-2,7,10,13,16,23-hexaazapentacosan-25-yl)-1,4,7,10-tetraazacyclododecan-1,4,7-triyl)triacetic acid (B-24)

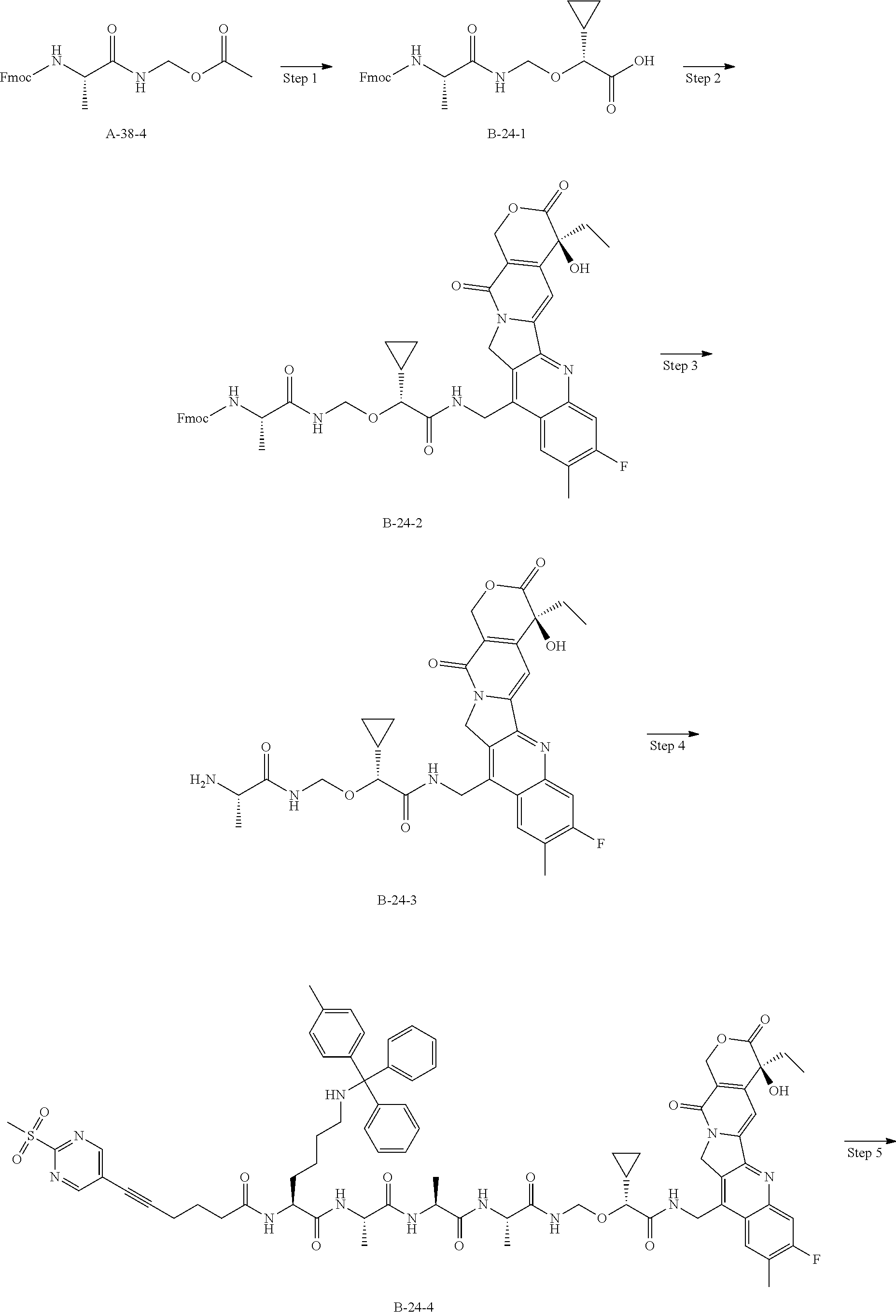

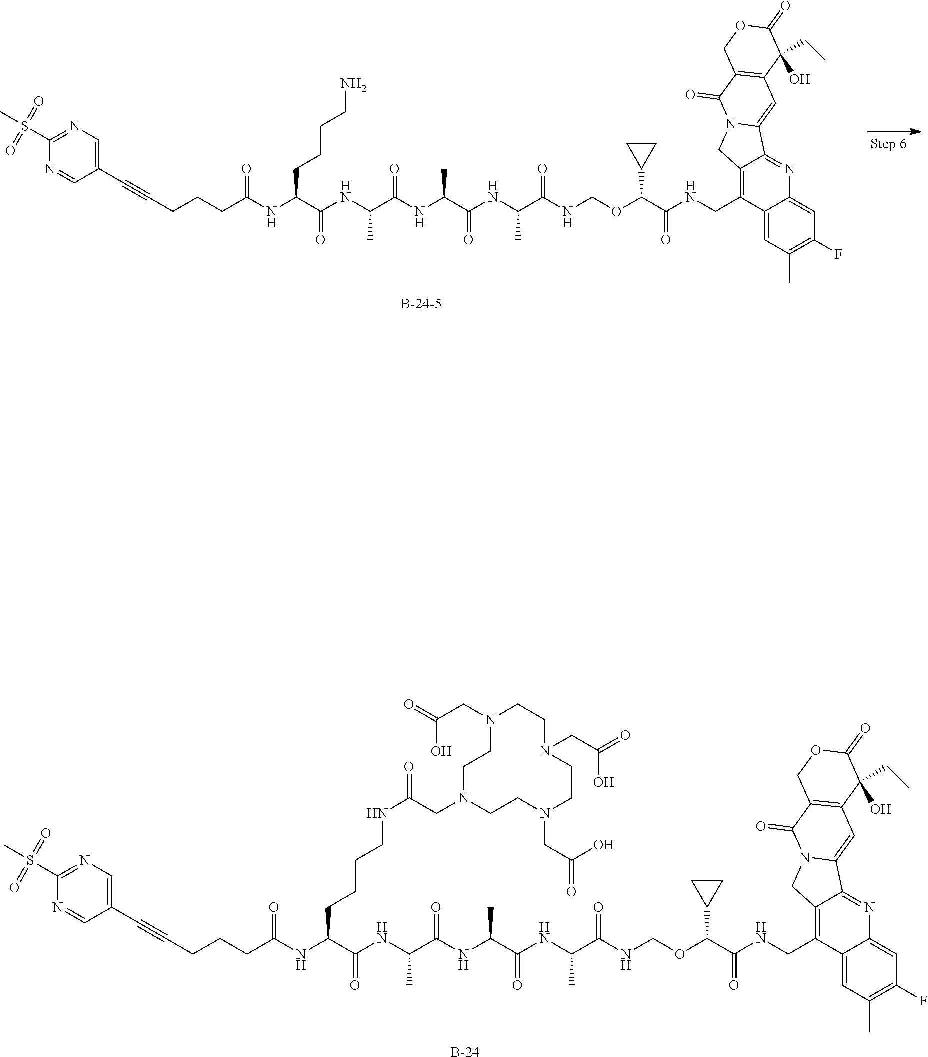

### Step 1: Preparation of (5S,10R)-10-cyclopropyl-1-(9H-fluoren-9-yl)-5-methyl-3,6-dioxo-2,9-dioxa-4,7-diazaundecan-11-oic acid (B-24-1)

Methyl (S)-(2-((((9H-fluoren-9-yl)methoxy)carbonyl)amino)propionamido)acetate (2 g, 5.23 mmol) and (R)-2-cyclopropyl-2-hydroxyacetic acid (1.34 g, 11.51 mmol) were dissolved in acetonitrile (100 mL), added with p-toluenesulfonic acid monohydrate (298.46 mg, 1.57 mmol), and stirred at room temperature for 0.5 hours. After the reaction was completed, the reaction solution was added with water and ethyl acetate for extraction, and the organic phase was dried and concentrated under reduced pressure to obtain a crude product. The crude product was purified by ammonium bicarbonate/acetonitrile system. The pH of the eluted phase was adjusted to 3-4 with formic acid, and the title compound (830 mg, 1.89 mmol) was obtained by extraction with ethyl acetate.

Its structural characterization data are as follows:

MS m/z (ESI): 439.18 [M+H]⁺

### Step 2: Preparation of (9H-fluoren-9-yl)methyl ((S)-1-((((R)-1-cyclopropyl-2-(((((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)amino)-2-oxoethoxy)methyl)amino)-1-oxopropan-2-yl)carbamate (B-24-2)

(5S,10R)-10-Cyclopropyl-1-(9H-fluoren-9-yl)-5-methyl-3,6-dioxo-2,9-dioxa-4,7-diazaundecan-11-oic acid (200 mg, 456.13 μmol), (S)-11-(aminomethyl)-4-ethyl-8-fluoro-4-hydroxy-9-methyl-1,12-dihydro-14H-pyrano[3′,4′:6,7]indolizino[1,2-b]quinolin-3,14(4H)-dione (230.58 mg, 456.13 μmol) and N,N-diisopropylethylamine (176.85 mg, 1.37 mmol) were dissolved in DMF (10 mL), added with HATU (260.16 mg, 684.21 μmol), and stirred at room temperature for 1 hour. After the reaction was completed, the reaction solution was directly purified by preparative purification to obtain the title compound (350 mg, 0.42 mmol).

Its structural characterization data are as follows:

MS m/z (ESI): 830.4 [M+H]⁺

Its preparation method was as follows:

Chromatographic column: Agilent Prep 1# C18
OBD (5 μm*19 mm*150 mm)
Mobile phase A: acetonitrile; Mobile phase B: water
(0.05% ammonium bicarbonate)

| Time | Mobile phase | Mobile phase | Flow rate |
|---|---|---|---|
| [min] | A [%] | B [%] | [mL/min] |
| 0.00 | 20 | 80 | 28 |
| 4.00 | 20 | 80 | 28 |
| 24.00 | 90 | 10 | 28 |

### Step 3: Preparation of (S)-2-amino-N—(((R)-1-cyclopropyl-2-(((((S)-4-ethyl-8-fluoro-4-hydroxy-9-methyl-3,14-dioxo)-3,4,12,14-tetrahydro-1H-pyrano[3′,4′:6,7]indolizino[1,2-b]quinolin-11-yl)methyl)amino)-2-oxoethoxy)methyl)propanamide (B-24-3)

(9H-Fluoren-9-yl)methyl ((S)-1-((((R)-1-cyclopropyl-2-((((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)amino)-2-oxoethoxy)methyl)amino)-1-oxopropan-2-yl)carbamate (350 mg, 421.75 μmol) was dissolved in DMF (5 mL), added with diethylamine (61.69 mg, 843.51 μmol), and stirred at room temperature for 1 hour. After the reaction was completed, the reaction solution was purified by flash column chromatography (C18, water/acetonitrile=2/1) and freeze-dried to obtain the title compound (192 mg, 0.32 mmol).

Its structural characterization data are as follows:

MS m/z (ESI): 608.3 [M+H]⁺

### Step 4: Preparation of N-((4R,9S,12S,15S,18S)-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-9,12,15-trimethyl-3,8,11,14,17-pentaoxo-24,24-diphenyl-24-(p-tolyl)-5-oxa-2,7,10,13,16,23-hexaazatetracosan-18-yl)-6-(2-(methylsulfonyl)pyrimidin-5-yl)hex-5-ynamide (B-24-4)

(S)-2-Amino-N—(((R)-1-cyclopropyl-2-((((S)-4-ethyl-8-fluoro-4-hydroxy-9-methyl-3,14-dioxo)-3,4,12,14-tetrahydro-1H-pyrano[3′,4′:6,7]indolizino[1,2-b]quinolin-11-yl)methyl)amino)-2-oxoethoxy)methyl)propanamide (93.37 mg, 153.66 μmol), N⁶-(diphenyl(p-tolyl)methyl)-N²-(6-(2-(methylsulfonyl)pyrimidin-5-yl)hex-5-ynoyl)-L-lysyl-L-alanyl-L-alanine (122.16 mg, 153.66 μmol), HATU (70.11 mg, 184.40 μmol) and N,N-diisopropylethylamine (59.58 mg, 460.99 μmol) were dissolved in N,N-dimethylformamide (5 mL). After the reaction was completed, the reaction solution was purified by reverse phase column chromatography to obtain the title compound (100 mg, 65.00 μmol).

Its structural characterization data are as follows:

MS m/z (ESI): 1384.7 [M+H]⁺

### Step 5: Preparation of N-((4R,9S,12S,15S,18S)-22-amino-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-9,12,15-trimethyl-3,8,11,14,17-pentaoxo-5-oxa-2,7,10,13,16-pentaazaeicosan-18-yl)-6-(2-(methylsulfonyl)pyrimidin-5-yl)hex-5-ynacetamide (B-24-5)

N-((4R,9S,12S,15S,18S)-4-Cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-9,12,15-trimethyl-3,8,11,14,17-pentaoxo-24,24-diphenyl-24-(p-tolyl)-5-oxa-2,7,10,13,16,23-hexaazatetracosan-18-yl)-6-(2-(methylsulfonyl)pyrimidin-5-yl)hex-5-ynamide (100 mg, 72.96 μmol) was dissolved in dichloromethane (3 mL), added with formic acid (3 mL), and stirred at room temperature for 1 hour. After the reaction was completed, the reaction solution was purified by preparative HPLC and freeze-dried to obtain the title compound (40 mg).

Its structural characterization data are as follows:

MS m/z (ESI): 1128.60 [M+H]⁺

Its preparation method was as follows:

Chromatographic column: Agilent Prep 1# C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 30 |
| 4.00 | 10 | 90 | 30 |
| 24.00 | 90 | 10 | 30 |

### Step 6: Preparation of 2,2′,2″-(10-((4R,9S,12S,15S,18S)-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-9,12,15-trimethyl-18-(6-(2-(methylsulfonyl)pyrimidin-5-yl)hex-5-ynamido)-3,8,11,14,17,24-hexaoxo-5-oxa-2,7,10,13,16,23-hexaazapentacosan-25-yl)-1,4,7,10-tetraazacyclododecan-1,4,7-triyl)triacetic acid (B-24)

N-((4R,9S,12S,15S,18S)-22-Amino-4-cyclopropyl-1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-9,12,15-trimethyl-3,8,11,14,17-pentaoxo-5-oxa-2,7,10,13,16-pentaazaeicosan-18-yl)-6-(2-(methylsulfonyl)pyrimidin-5-yl)hex-5-ynacetamide (40 mg, 35.45 μmol) and 2,2′,2″-(10-(2-((2,5-dioxopyrrolidin-1-yl)oxy)-2-oxoethyl)-1,4,7,10-tetraazacyclododecan-1,4,7-triyl)triacetic acid (21.34 mg, 42.54 μmol) were dissolved in N,N-dimethylformamide (2 mL), and added with N,N-diisopropylethylamine (9.16 mg, 70.91 μmol). The reaction was carried out under stirring at room temperature for 1 hour. After the reaction was completed, the reaction solution was purified and freeze-dried to obtain the title compound (28.29 mg, 17.74 μmol).

Its structural characterization data are as follows:

MS m/z (ESI): 1515.7 [M+H]⁺

Its preparation method was as follows:

Chromatographic column: Waters Xbridge Prep C18
OBD (5 μm*19 mm*150 mm)
Mobile phase A: acetonitrile; Mobile phase B: water (0.05% formic acid)

| Time | Mobile phase | Mobile phase | Flow rate |
|---|---|---|---|
| [min] | A [%] | B [%] | [mL/min] |
| 0.00 | 10 | 90 | 28 |
| 2.00 | 10 | 90 | 28 |
| 18.00 | 80 | 20 | 28 |

### II. Preparation of Antibodies

According to the amino acid sequence of Trastuzumab (IMGT/mAb-DB ID: 97) and the amino acid sequence of Pertuzumab (IMGT/mAb-DB ID: 80) in the IMGT database, codon optimization was performed and the coding genes were synthesized, constructed into the expression vectors, transfected into CHO cells and screened under pressure to construct stable expression cell lines. After expression, the supernatants were collected, and the corresponding antibodies Trastuzumab and Pertuzumab were purified by Protein A affinity filler. The information of amino acid sequences of Trastuzumab and Pertuzumab is shown above.

III. Conjugation of Compounds Containing Cell Bioactive Molecules and Linkers with Antibodies

The conjugation preparation of antibody-drug conjugate samples was shown as follows:

### 1. Preparation of Trastuzumab-A-1

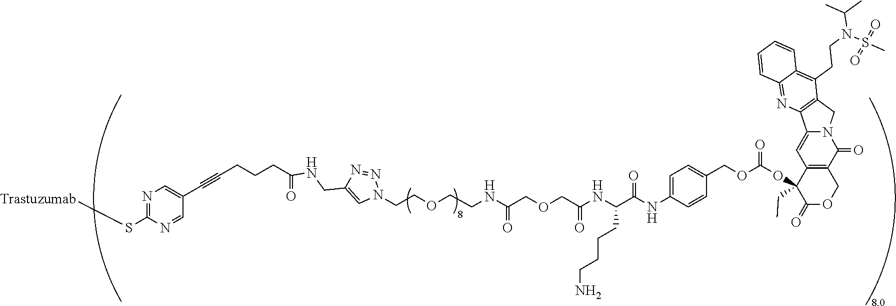

3.086 mL of Trastuzumab antibody (16.2 mg/mL) was taken, diluted with 154 μL of 20 mM PB+0.1M EDTA (pH 7.60), then adjusted to pH 7.60 with 1M Na₂HPO₄ solution, added with 10 mM TCEP (tris(2-carboxyethyl)phosphine, 189.47 uL, pH 7.60) solution, mixed well, and allowed to stand at room temperature for 1.5 hours. Then A-1 (prepared according to the method of Example 14 of WO2019114666, 351.5 μL, 10 mM) that was in an amount 10 times the mole number of the antibody and dissolved in dimethyl sulfoxide solution was added, mixed well, and allowed to stand at room temperature for 2 hours. After the above operations were completed, NAP-5 gel column (Cytiva) was used to replace the buffer with 20 mM histidine buffer solution at pH 6.0 to obtain an antibody-drug conjugate (i.e., Trastuzumab-A-1). The DAR value determined by mass spectrometry was 8.0.

### 2. Preparation of Trastuzumab-A-2

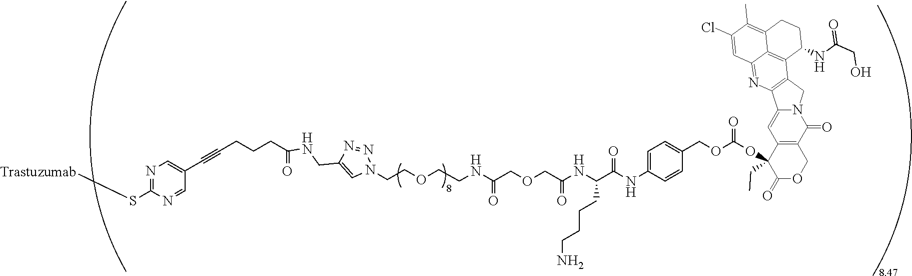

0.617 of mL of Trastuzumab antibody (16.2 mg/mL) was taken, diluted with 30.86 μL of 20 mM PB+0.1M EDTA (pH 7.60), then adjusted to pH 7.60 with 1M Na₂HPO₄ solution, added with 10 mM TCEP (tris(2-carboxyethyl)phosphine, 37.89 μL, pH 7.60) solution, mixed well, and allowed to stand at room temperature for 1.5 hours. Then A-2 (84.36 μL, 10 mM) that was in an amount 12 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-2). The DAR value determined by mass spectrometry was 8.47.

### 3. Preparation of Pertuzumab-A-2

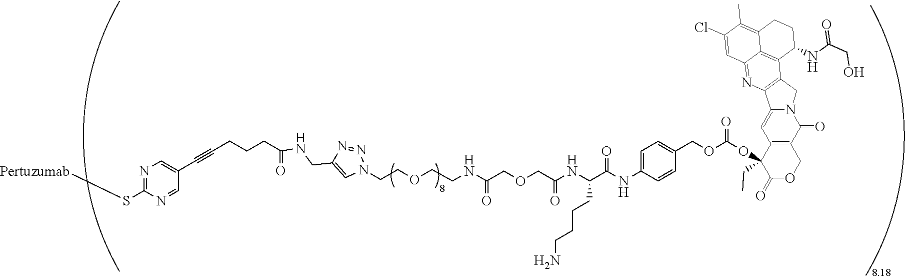

2.276 mL of Pertuzumab antibody (13.18 mg/mL) was taken, diluted with 113.8 μL of 20 mM PB+0.1M EDTA (pH 7.60), then adjusted to pH 7.70 with 1M Na₂HPO₄ solution, added with 10 mM TCEP (tri(2-carboxyethyl)phosphine, 111.5 μL, pH 7.60) solution, mixed well, and allowed to stand at room temperature for 1.5 hours. Then A-2 (227.55 μL, 10 mM) that was in an amount 11 times the mole number of the antibody and dissolved in dimethyl sulfoxide solution was added, mixed well, and allowed to stand at room temperature for 2 hours. After the above operations were completed, NAP-5 gel column (Cytiva) was used to replace the buffer with 20 mM histidine buffer solution at pH 6.0 to obtain an antibody-drug conjugate (i.e., Pertuzumab-A-2). The DAR value determined by mass spectrometry was 8.18.

### 4. Preparation of Trastuzumab-A-14

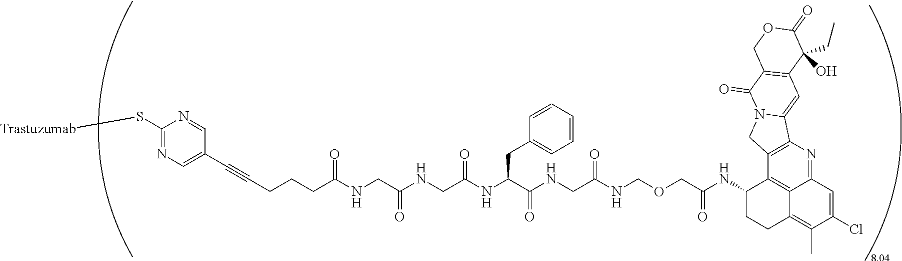

2.469 mL of Trastuzumab antibody (16.2 mg/mL) was taken, diluted with 123 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 151.57 μL, pH 7.60) solution, mixed well, and allowed to stand at room temperature for 1.5 hours. Then, A-14 (290.09 μL, 10 mM) that was in an amount 10 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-14). The DAR value determined by mass spectrometry was 8.04.

### 5. Preparation of Pertuzumab-A-14

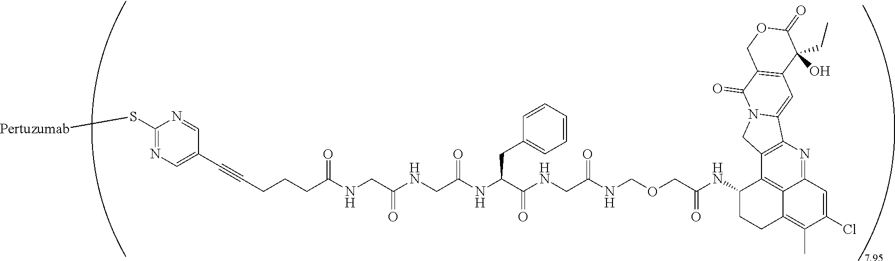

2.276 mL of Pertuzumab antibody (13.18 mg/mL) was taken, diluted with 113.8 μL of 20 mM PB+0.1 M EDTA (pH 7.60), then adjusted to pH 7.70 with 1 M Na₂HPO₄ solution, added with 10 mM TCEP (tri(2-carboxyethyl)phosphine, 111.5 μL, pH 7.60) solution, mixed well, and allowed to stand at room temperature for 1.5 hours. A-14 (213.4 μL, 10 mM) that was in an amount 10 times the mole number of the antibody and dissolved in dimethyl sulfoxide solution was added, mixed well, and allowed to stand at room temperature for 2 hours. Then NAP-5 gel column (Cytiva) was used to replace the buffer with 20 mM histidine buffer solution at pH 6.0 to obtain an antibody-drug conjugate (i.e., Pertuzumab-A-14). The DAR value determined by mass spectrometry was 7.95.

### 6. Preparation of Trastuzumab-A-26

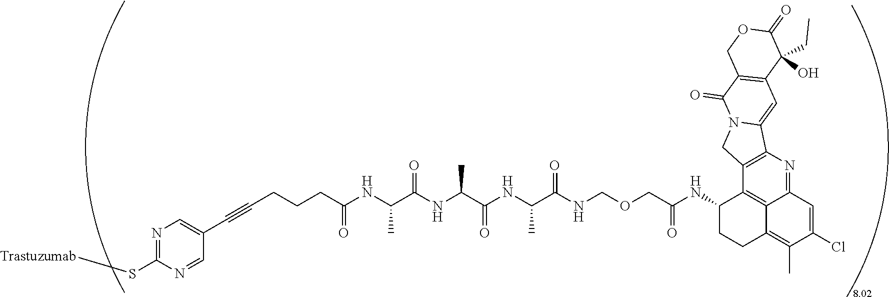

2.564 mL of Trastuzumab antibody (15.6 mg/mL) was taken, diluted with 128.2 μL of 20 mM PB+0.1M EDTA (pH 7.60), then adjusted to pH 7.60 with 1M Na₂HPO₄ solution, added with 10 mM TCEP (tri(2-carboxyethyl)phosphine, 151.57 μL, pH 7.60) solution, mixed well, and allowed to stand at room temperature for 1.5 hours. Then A-26 (290.1 μL, 10 mM) that was in an amount 10 times the mole number of the antibody and dissolved in dimethyl sulfoxide solution was added, mixed well, and allowed to stand at room temperature for 2 hours. Then the buffer was replaced with a 20 mM histidine buffer solution at pH 6.0 using NAP-5 gel column (Cytiva) to obtain an antibody-drug conjugate (i.e., Trastuzumab-A-26). The DAR value determined by mass spectrometry was 8.02.

### 7. Preparation of Pertuzumab-A-26

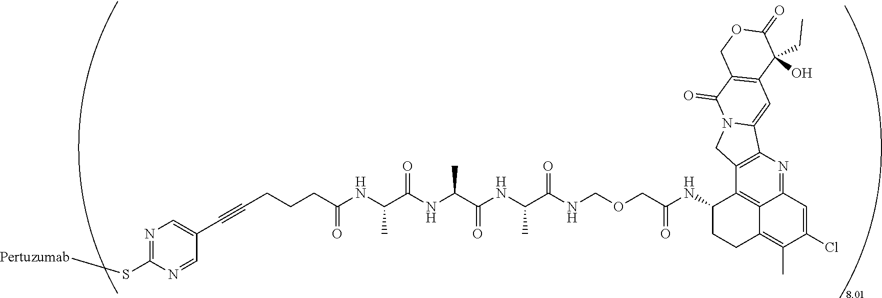

2.276 mL of Pertuzumab antibody (13.18 mg/mL) was taken, diluted with 113.8 μL of 20 mM PB+0.1M EDTA (pH 7.60), then adjusted to pH 7.70 with 1M Na₂HPO₄ solution, added with 10 mM TCEP (tris(2-carboxyethyl)phosphine, 111.5 μL, pH 7.60), mixed well, and allowed to stand at room temperature for 1.5 hours. A-26 (206.86 μL, 10 mM) that was in an amount 10 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Pertuzumab-A-26). The DAR value determined by mass spectrometry was 8.01.

### 8. Preparation of Trastuzumab-A-36

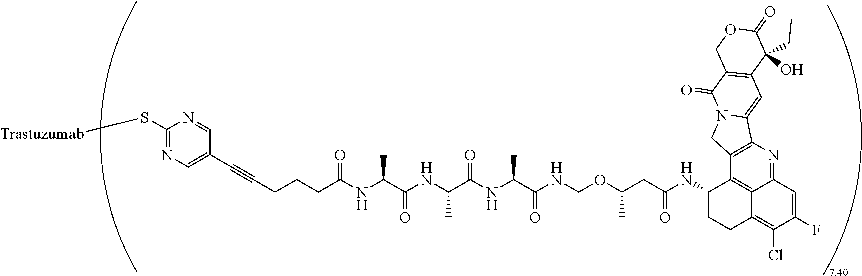

0.423 mL of Trastuzumab antibody (4.73 mg/mL) was taken, diluted with 21.17 μL of 20 mM PB+0.1 M EDTA (pH 7.60), then adjusted to pH 7.60 with 1 M Na₂HPO₄ solution, added with 10 mM TCEP (tri(2-carboxyethyl)phosphine, 7.58 μL, pH 7.60) solution, mixed well, and allowed to stand at room temperature for 1.5 hours. Then A-36 (14.21 μL, 10 mM) that was in an amount 10 times the mole number of the antibody and dissolved in dimethyl sulfoxide solution was added, mixed well, and allowed to stand at room temperature for 2 hours. After the above operations were completed, NAP-5 gel column (Cytiva) was used to replace the buffer with 20 mM histidine buffer solution at pH 6.0 to obtain an antibody-drug conjugate (i.e., Trastuzumab-A-36). The DAR value determined by mass spectrometry was 7.40.

### 9. Preparation of Trastuzumab-A-38

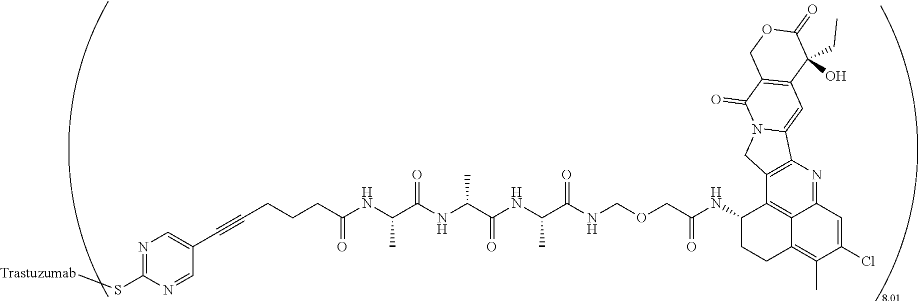

0.617 mL of Trastuzumab antibody (16.2 mg/mL) was taken, diluted with 30.86 μL of 20 mM PB+0.1M EDTA (pH 7.60), adjusted to pH 7.60 with 1M Na₂HPO₄ solution, added with 10 mM TCEP (tri(2-carboxyethyl)phosphine, 37.89 μL, pH 7.60) solution, mixed well, and allowed to stand at room temperature for 1.5 hours. Then A-38 (101.52 μL, 10 mM) that was in an amount 14 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-38). The DAR value determined by mass spectrometry was 8.01.

### 10. Preparation of Trastuzumab-A-40

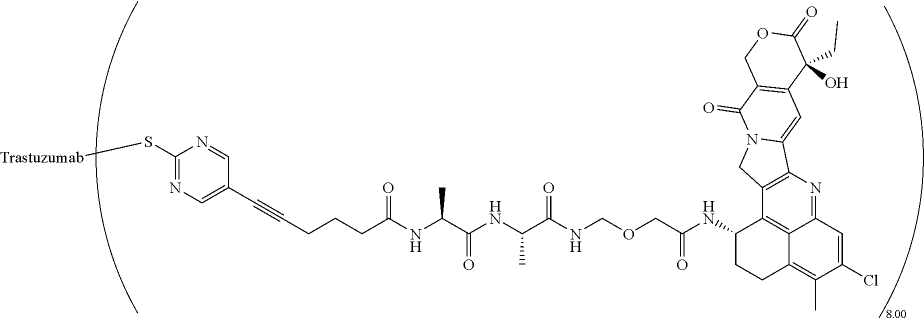

0.617 mL of Trastuzumab antibody (16.2 mg/mL) was taken, diluted with 30.86 μL of 20 mM PB+0.1M EDTA (pH 7.60), then adjusted pH to 7.60 with 1M Na₂HPO₄ solution, added with 10 mM TCEP (tris(2-carboxyethyl)phosphine, 37.89 μL, pH 7.60) solution, mixed well, and allowed to stand at room temperature for 1.5 hours. Then A-40 (73.3 μL, 10 mM) that was in an amount 10 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-40). The DAR value determined by mass spectrometry was 8.00.

### 11. Preparation of Trastuzumab-A-42

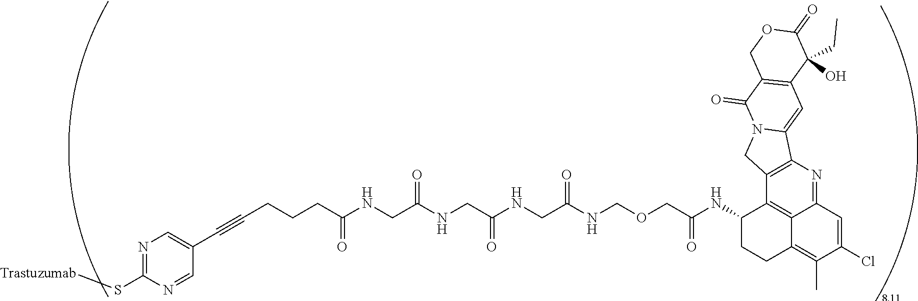

2.469 mL of Trastuzumab antibody (16.2 mg/mL) was taken, diluted with 123 μL of 20 mM PB+0.1M EDTA (pH 7.60), then adjusted to pH 7.60 with 1M Na₂HPO₄ solution, added with 10 mM TCEP (tris(2-carboxyethyl)phosphine, 151.57 μL, pH 7.60) solution, mixed well, and allowed to stand at room temperature for 1.5 hours. Then A-42 (278.37 μL, 10 mM) that was in an amount 10 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-42). The DAR value determined by mass spectrometry was 8.11.

### 12. Preparation of Trastuzumab-A-44

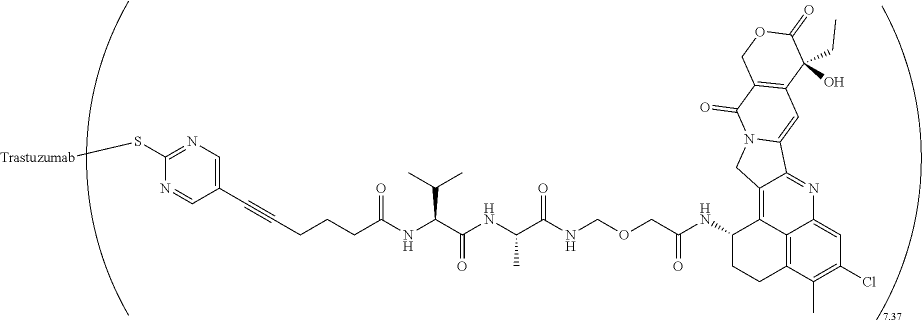

0.617 mL of Trastuzumab antibody (16.2 mg/mL) was taken, diluted with 30.86 μL of 20 mM PB+0.1 M EDTA (pH 7.60), then adjusted to pH 7.60 with 1M Na₂HPO₄ solution, added with 10 mM TCEP (tri(2-carboxyethyl)phosphine, 37.89 μL, pH 7.60) solution, mix well, and allowed to stand at room temperature for 1.5 hours. Then A-44 (98.42 μL, 10 mM) that was in an amount 14 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-44). The DAR value determined by mass spectrometry was 7.37.

### 13. Preparation of Pertuzumab-A-44

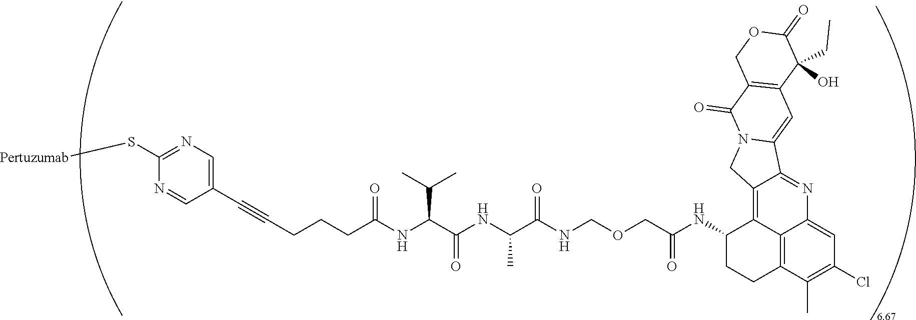

2.276 mL of Pertuzumab antibody (13.18 mg/mL) was taken, diluted with 113.8 μL of 20 mM PB+0.1MEDTA (pH 7.60), then adjusted to pH 7.70 with 1M Na₂HPO₄ solution, added with 10 mM TCEP (tris(2-carboxyethyl)phosphine, 111.5 μL, pH 7.60) solution, mixed well, and allowed to stand at room temperature for 1.5 hours. Then A-44 (289.6 μL, 10 mM) that was in an amount 14 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Pertuzumab-A-44). The DAR value determined by mass spectrometry was 6.67.

### 14. Preparation of Trastuzumab-A-46

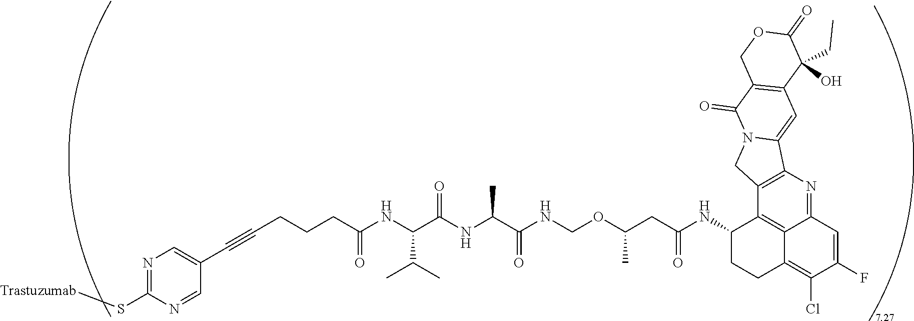

0.423 mL of Trastuzumab antibody (4.73 mg/mL) was taken, diluted with 21.17 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 7.58 μL, pH 7.60) solution, mixed well, and allowed to stand at room temperature for 1.5 hours. Then A-46 (14.21 μL, 10 mM) that was in an amount 10 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-46). The DAR value determined by mass spectrometry was 7.27.

### 15. Preparation of Trastuzumab-A-52

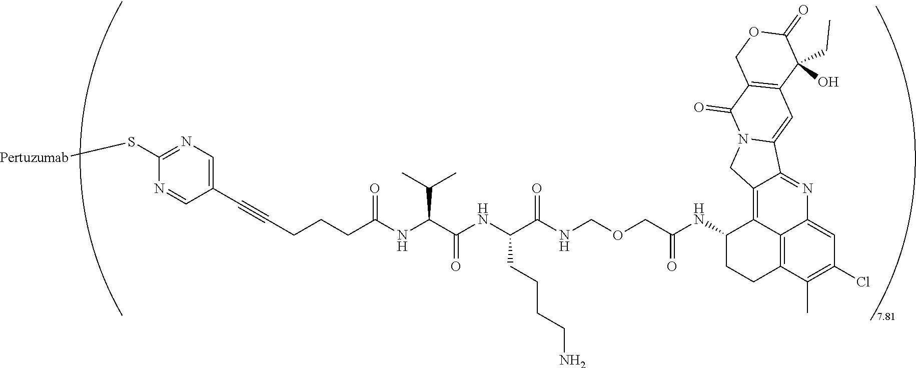

0.617 mL of Trastuzumab antibody (16.2 mg/mL) was taken, diluted with 30.86 μL of 20 mM PB+0.1M EDTA (pH 7.60), then adjusted to pH 7.60 with 1M Na₂HPO₄ solution, added with 10 mM TCEP (tris(2-carboxyethyl)phosphine, 37.89 μL, pH 7.60) solution, mixed well, and allowed to stand at room temperature for 1.5 hours. Then, A-52 (71.77 μL, 10 mM) that was in an amount 10 times the mole number of the antibody and dissolved in dimethyl sulfoxide solution was slowly added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-52). The DAR value determined by mass spectrometry was 7.81.

### 16. Preparation of Trastuzumab-A-54

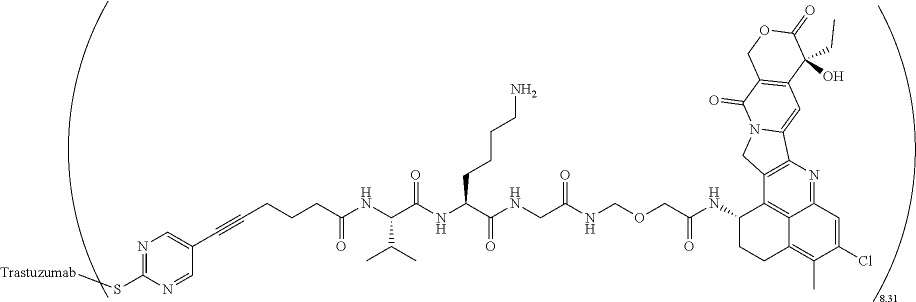

2.469 mL of Trastuzumab antibody (16.2 mg/mL) was taken, diluted with 123 μL of 20 mM PB+0.1 M EDTA (pH 7.60), then adjusted to pH 7.60 with 1M Na₂HPO₄ solution, added with 10 mM TCEP (tri(2-carboxyethyl)phosphine, 151.57 μL, pH 7.60) solution, mixed well, and allowed to stand at room temperature for 1.5 hours. Then A-54 (315.77 μL, 10 mM) that was in an amount 11 times the mole number of the antibody and dissolved in dimethyl sulfoxide solution was slowly added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-54). The DAR value determined by mass spectrometry was 8.31.

### 17. Preparation of Trastuzumab-A-62

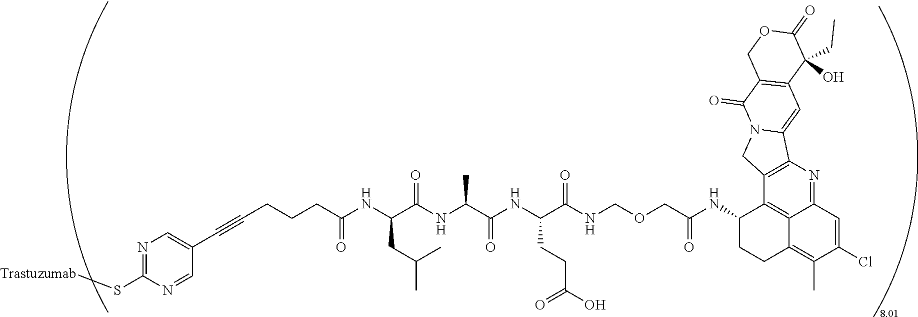

0.617 mL of Trastuzumab antibody (16.2 mg/mL) was taken, diluted with 30.86 μL of 20 mM PB+0.1M EDTA (pH 7.60), then adjusted to pH 7.60 with 1M Na₂HPO₄ solution, added with 10 mM TCEP (tri(2-carboxyethyl)phosphine, 37.89 μL, pH 7.60) solution, mixed well, and allowed to stand at room temperature for 1.5 hours. Then A-62 (72.52 μL, 10 mM) that was in an amount 10 times the mole number of the antibody and dissolved in dimethyl sulfoxide solution was slowly added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-62). The DAR value determined by mass spectrometry was 8.01.

### 18. Preparation of Pertuzumab-A-62

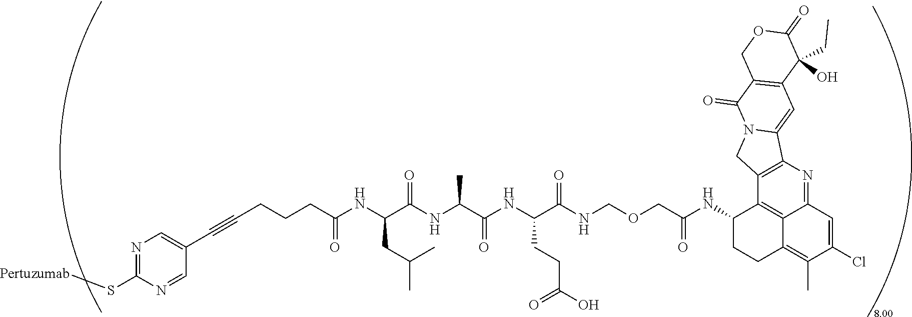

2.276 mL of Pertuzumab antibody (13.18 mg/mL) was taken, diluted with 113.8 μL of 20 mM PB+0.1 M EDTA (pH 7.60), then adjusted to pH 7.70 with 1 M Na₂HPO₄ solution, added with 10 mM TCEP (tris(2-carboxyethyl)phosphine, 111.5 μL, pH 7.60) solution, mixed well, and allowed to stand at room temperature for 1.5 hours. Then A-62 (213.4 μL, 10 mM) that was in an amount 10 times the mole number of the antibody and dissolved in dimethyl sulfoxide solution was added, mixed well, and allowed to stand at room temperature for 2 hours. After the above operations were completed, NAP-5 gel column (Cytiva) was used to replace the buffer with 20 mM histidine buffer solution at pH 6.0 to obtain an antibody-drug conjugate (i.e., Pertuzumab-A-62). The DAR value determined by mass spectrometry was 8.00.

### 19. Preparation of Trastuzumab-A-66

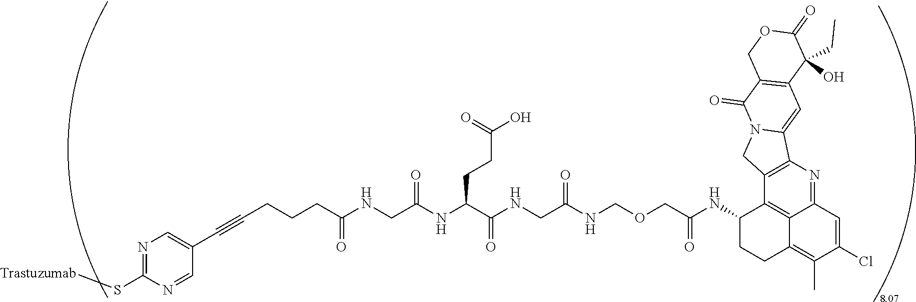

0.309 mL of Trastuzumab antibody (16.2 mg/mL) was taken, diluted with 15.4 μL of 20 mM PB+0.1M EDTA (pH 7.60), then adjusted to pH 7.60 with 1M Na₂HPO₄ solution, added with 10 mM TCEP (tris(2-carboxyethyl)phosphine, 18.95 μL, pH 7.60) solution, mixed well, and allowed to stand at room temperature for 1.5 hours. Then A-66 (34.83 μL, 10 mM) that was in an amount 10 times the mole number of the antibody and dissolved in dimethyl sulfoxide solution was slowly added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-66). The DAR value determined by mass spectrometry was 8.07.

### 20. Preparation of Trastuzumab-A-68

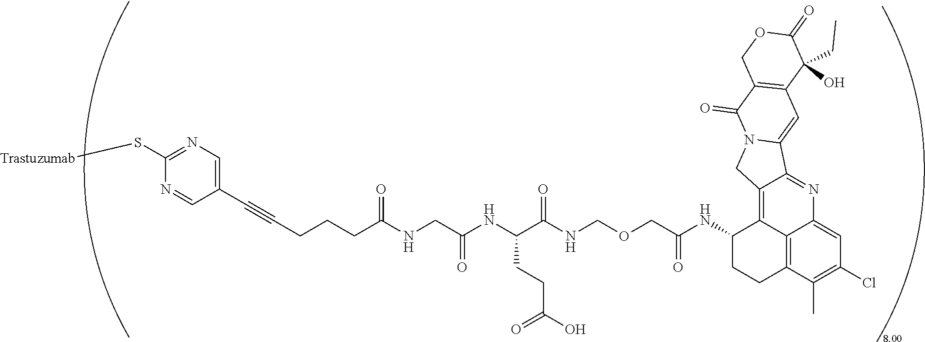

0.617 mL of Trastuzumab antibody (16.2 mg/mL) was taken, diluted with 30.86 μL 20 mM PB+0.1M EDTA (pH 7.60), then adjusted to pH 7.60 with 1M Na₂HPO₄ solution, added with 10 mM TCEP (tri(2-carboxyethyl)phosphine, 37.89 μL, pH 7.60) solution, mixed well, and allowed to stand at room temperature for 1.5 hours. Then A-68 (71.77 μL, 10 mM) that was in an amount 10 times the mole number of the antibody and dissolved in dimethyl sulfoxide solution was slowly added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-68). The DAR value determined by mass spectrometry was 8.00.

### 21. Preparation of Trastuzumab-A-70

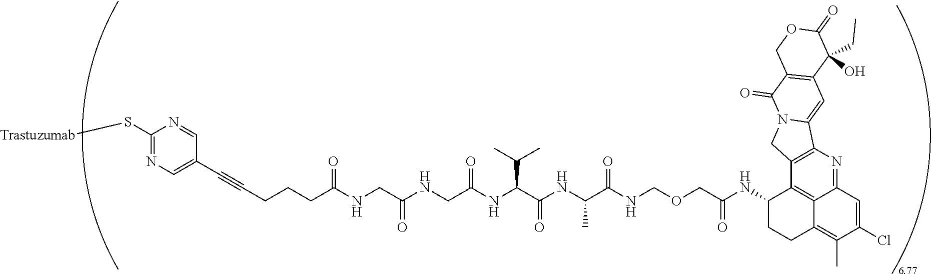

0.423 mL of Trastuzumab antibody (4.73 mg/mL) was taken, diluted with 21.17 μL of 20 mM PB+0.1M EDTA (pH 7.60), then adjusted to pH 7.60 with 1M Na₂HPO₄ solution, added with 10 mM TCEP (tris(2-carboxyethyl)phosphine, 7.58 μL, pH 7.60) solution, mixed well, and allowed to stand at room temperature for 1.5 hours. Then A-70 (13.92 μL, 10 mM) that was in an amount 10 times the mole number of the antibody and dissolved in dimethyl sulfoxide solution was added, mixed well, and allowed to stand at room temperature for 2 hours. After the above operations were completed, NAP-5 gel column (Cytiva) was used to replace the buffer with 20 mM histidine buffer solution at pH 6.0 to obtain an antibody-drug conjugate (i.e., Trastuzumab-A-70). The DAR value determined by mass spectrometry was 6.77.

### 22. Preparation of Trastuzumab-A-72

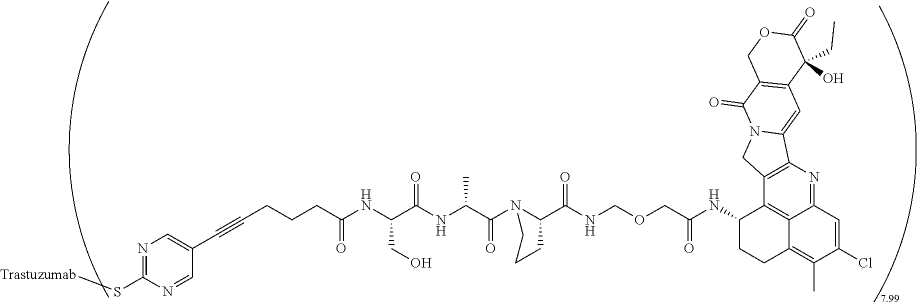

0.123 mL of Trastuzumab antibody (16.2 mg/mL) was taken, diluted with 6.15 μL of 20 mM PB+0.1M EDTA (pH 7.60), then adjusted to pH 7.60 with 1M Na₂HPO₄ solution, added with 10 mM TCEP (tris(2-carboxyethyl)phosphine, 7.58 μL, pH 7.60) solution, mixed well, and allowed to stand at room temperature for 1.5 hours. Then A-72 (14.51 μL, 10 mM) that was in an amount 10 times the mole number of the antibody and dissolved in dimethyl sulfoxide solution was added slowly, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-72). The DAR value determined by mass spectrometry was 7.99.

### 23. Preparation of Trastuzumab-A-76

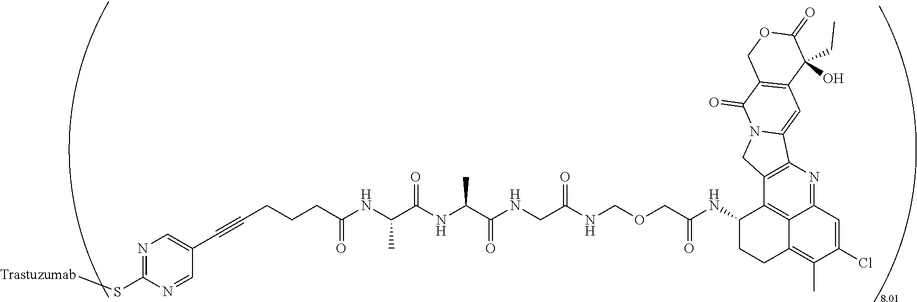

0.617 mL of Trastuzumab antibody (16.2 mg/mL) was taken, diluted with 30.86 μL of 20 mM PB+0.1M EDTA (pH 7.60), then adjusted to pH 7.60 with 1M Na₂HPO₄ solution, added with 10 mM TCEP (tri(2-carboxyethyl)phosphine, 37.89 μL, pH 7.60) solution, mixed well, and allowed to stand at room temperature for 1.5 hours. Then A-76 (73.5 μL, 10 mM) that was in an amount 10 times the mole number of the antibody and dissolved in dimethyl sulfoxide solution was slowly added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-76). The DAR value determined by mass spectrometry was 8.01.

### 24. Preparation of Trastuzumab-A-78

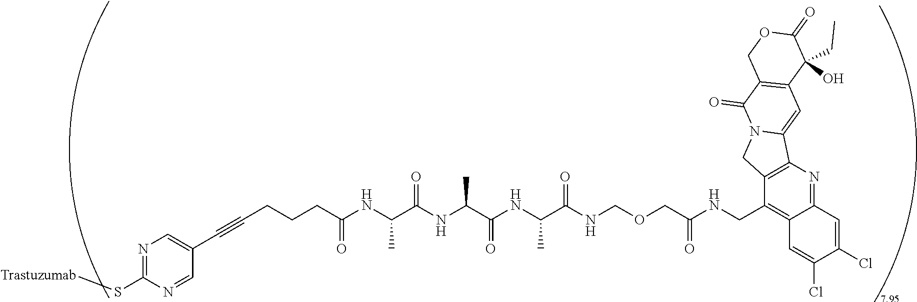

2.16 mL of Trastuzumab antibody (16.2 mg/mL) was taken, diluted with 108 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 88.42 μL, pH 7.60) solution, mixed well, and allowed to stand at room temperature for 1.5 hours. Then A-78 (502.38 μL, 10 mM) that was in an amount 20 times the mole number of the antibody and dissolved in dimethyl sulfoxide solution was added slowly, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-78). The DAR value determined by mass spectrometry was 7.95.

### 25. Preparation of Trastuzumab-A-80

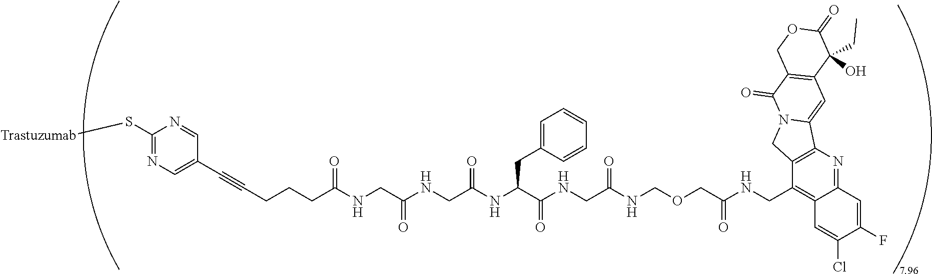

1.973 mL of Trastuzumab antibody (15.2 mg/mL) was taken, diluted with 98.7 μL of 20 mM PB+0.1M EDTA (pH 7.60), then adjusted to pH 7.60 with 1M Na₂HPO₄ solution, added with 10 mM TCEP (tris(2-carboxyethyl)phosphine, 56.92 μL, pH 7.60) solution, mixed well, and allowed to stand at room temperature for 1.5 hours. Then A-80 (258.16 μL, 10 mM) that was in an amount 12 times the mole number of the antibody and dissolved in dimethyl sulfoxide solution was slowly added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-80). The DAR value determined by mass spectrometry was 7.96.

### 26. Preparation of Trastuzumab-A-84

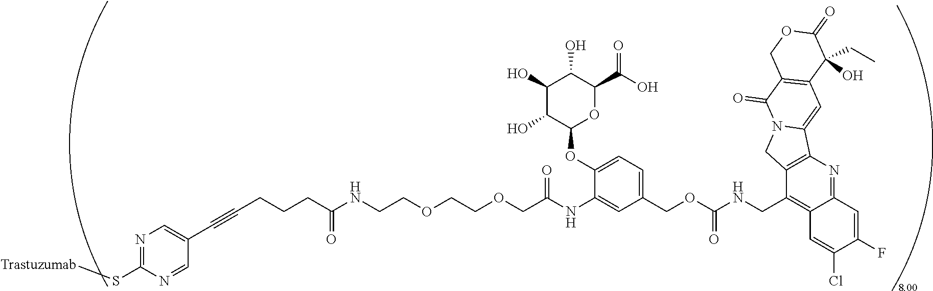

1.781 mL of Trastuzumab antibody (16 mg/mL) was taken, diluted with 89 μL of 20 mM PB+0.1M EDTA (pH 7.60), then adjusted to pH 7.60 with 1M Na₂HPO₄ solution, added with 10 mM TCEP (tris(2-carboxyethyl)phosphine, 108 μL, pH 7.60) solution, mixed well, and allowed to stand at room temperature for 1.5 hours. Then A-84 (218 μL, 10 mM) that was in an amount 10 times the mole number of the antibody and dissolved in dimethyl sulfoxide solution was added slowly, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-84). The DAR value determined by mass spectrometry was 8.00.

### 27. Preparation of Trastuzumab-A-88

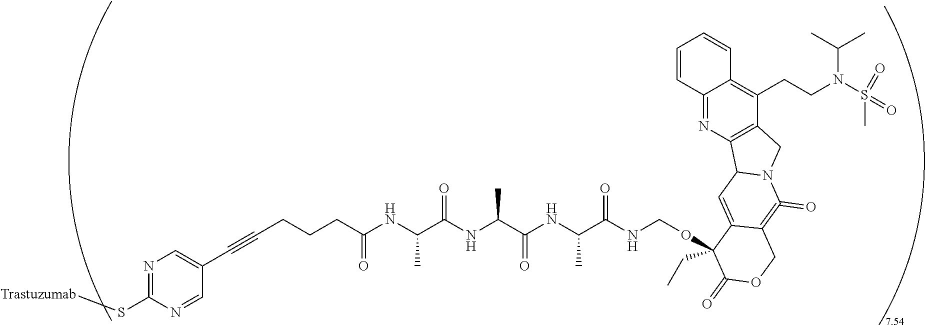

1.234 mL of Trastuzumab antibody (16.2 mg/mL) was taken, diluted with 61.73 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 75.8 μL, pH 7.60) solution, mixed well, and allowed to stand at room temperature for 1.5 hours. Then A-88 (143.56 μL, 10 mM) that was in an amount 10 times the mole number of the antibody and dissolved in dimethyl sulfoxide solution was added, mixed well, and allowed to stand at room temperature for 2 hours. After the above operations were completed, NAP-5 gel column (Cytiva) was used to replace the buffer with 20 mM histidine buffer solution at pH 6.0 to obtain an antibody-drug conjugate (i.e., Trastuzumab-A-88). The DAR value determined by mass spectrometry was 7.54.

### 28. Preparation of Trastuzumab-A-89

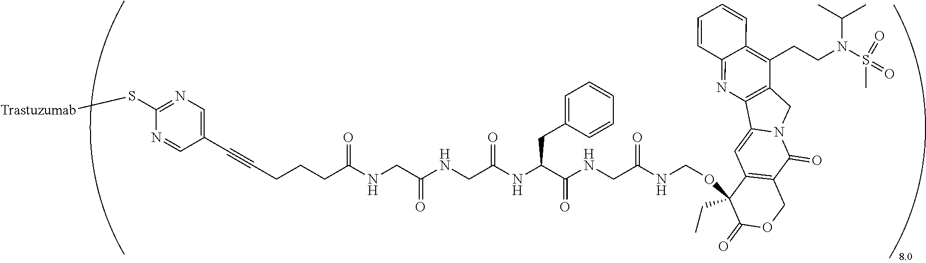

1.234 mL of Trastuzumab antibody (16.2 mg/mL) was taken, diluted with 61.73 μL of 20 mM PB+0.1M EDTA (pH 7.60), then adjusted to pH 7.60 with 1M Na₂HPO₄ solution, added with 10 mM TCEP (tris (2-carboxyethyl) phosphine, 75.8 μL, pH 7.60) solution, mixed well, and allowed to stand at room temperature for 1.5 hours. Then A-89 solution (153 μL, 10 mM) that was in an amount 11 times the mole number of the antibody and dissolved in dimethyl sulfoxide solution was added, mixed well, and allowed to stand at room temperature for 2 hours. After the above operations were completed, the buffer was replaced with a 20 mM histidine buffer solution at pH 6.0 using NAP-5 gel column (Cytiva) to obtain an antibody-drug conjugate (i.e., Trastuzumab-A-89). The DAR value determined by mass spectrometry was 8.0.

### 29. Preparation of Trastuzumab-A-97

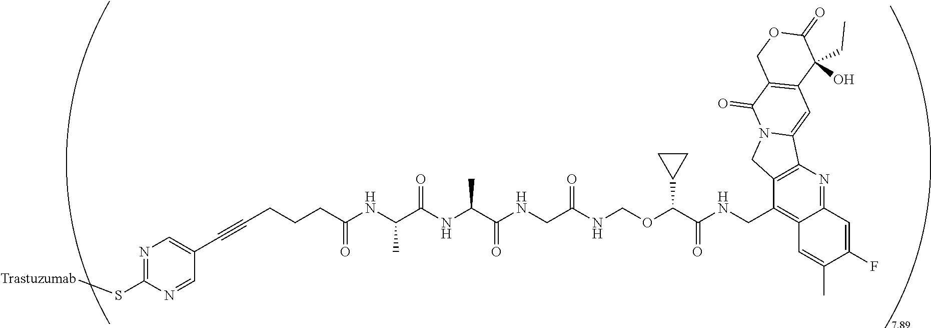

2.041 mL of Trastuzumab (14.7 mg/mL) was taken, diluted with 102 μL of 20 mM PB+0.1M EDTA (pH 7.60), then adjusted to pH 7.6 with 1M Na₂HPO₄ solution, added with 10 mM TCEP (tri(2-carboxyethyl)phosphine, 113.7 μL, pH 7.60) solution, mixed well, and allowed to stand at room temperature for 1.5 hours. Then A-97 (213.1 μL, 10 mM) that was in an amount 10 times the mole number of the antibody and dissolved in dimethyl sulfoxide solution was added, mixed well, and allowed to stand at room temperature for 2 h. Then NAP gel column (Cytiva) was used to replace the buffer with 20 mM histidine buffer solution at pH 6.0 to obtain an antibody-drug conjugate (i.e., Trastuzumab-A-97). The DAR value determined by mass spectrometry was 7.89.

### 30. Preparation of Trastuzumab-A-98

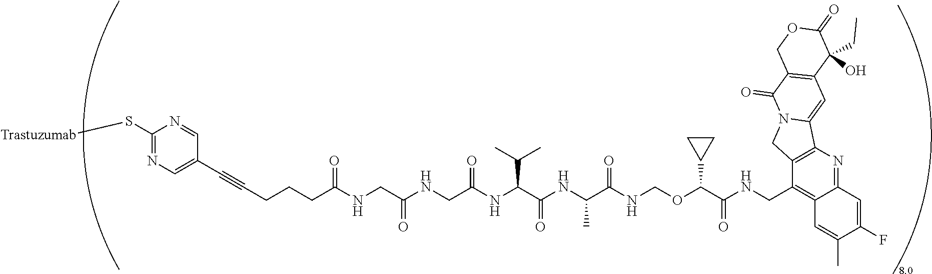

2.041 mL of Trastuzumab (14.7 mg/mL) was taken, diluted with 102 μL of 20 mM PB+0.1M EDTA (pH 7.60), then adjusted to pH 7.6 with 1M Na₂HPO₄ solution, added with 10 mM TCEP (tri(2-carboxyethyl)phosphine, 113.7 μL, pH 7.60) solution, mixed well, and allowed to stand at room temperature for 1.5 hours. Then A-98 (215.3 μL, 10 mM) that was in an amount 10 times the mole number of the antibody and dissolved in dimethyl sulfoxide solution was added, mixed well, and allowed to stand at room temperature for 2 h. Then NAP gel column (Cytiva) was used to replace the buffer with 20 mM histidine buffer solution at pH 6.0 to obtain an antibody-drug conjugate (i.e., Trastuzumab-A-98). The DAR value determined by mass spectrometry was 8.0.

### 31. Preparation of Trastuzumab-A-99

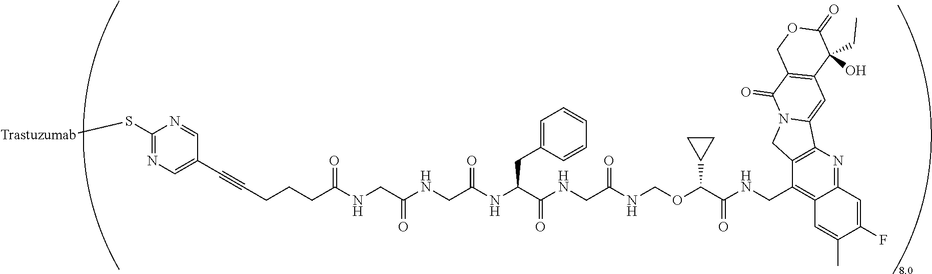

2.041 mL of Trastuzumab (14.7 mg/mL) was taken, diluted with 102 μL of 20 mM PB+0.1 M EDTA (pH 7.60), then adjusted to pH 7.6 with 1 M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then A-99 (213.1 μL, 10 mM) that was in an amount 10 times the mole number of the antibody and dissolved in dimethyl sulfoxide solution was added, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-A-99). The DAR value determined by mass spectrometry was 8.0.

### 32. Preparation of Trastuzumab-A-126

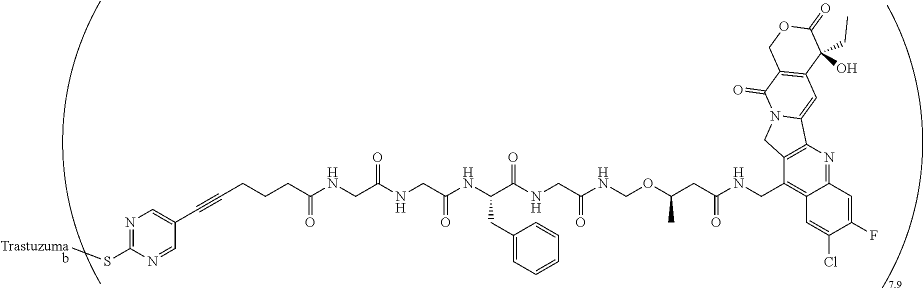

2.333 mL of Trastuzumab (15 mg/mL) was taken, diluted with 117 μL of 20 mM PB+0.1M EDTA (pH 7.60), then adjusted to pH 7.60 with 1M Na₂HPO₄ solution, added with 10 mM TCEP (tri(2-carboxyethyl)phosphine, 132.6 μL, pH 7.60) solution, mixed well, and allowed to stand at room temperature for 1.5 hours. Then A-126 (273.5 μL, 10 mM) that was in an amount 11 times the mole number of the antibody and dissolved in dimethyl sulfoxide solution was added, mixed well, and allowed to stand at room temperature for 2 h. Then NAP gel column (Cytiva) was used to replace the buffer with 20 mM histidine buffer solution at pH 6.0 to obtain an antibody-drug conjugate (i.e., Trastuzumab-A-126). The DAR value determined by mass spectrometry was 7.9.

### 33. Preparation of Trastuzumab-A-136

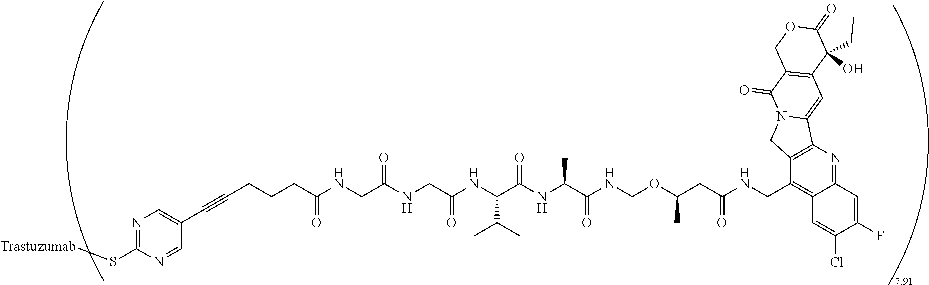

2.333 mL of Trastuzumab (15 mg/mL) was taken, diluted with 117 μL of 20 mM PB+0.1M EDTA (pH 7.60), then adjusted to pH 7.60 with 1M Na₂HPO₄ solution, added 10 mM TCEP (tris(2-carboxyethyl)phosphine, 132.6 μL, pH 7.60) solution, mixed well, and allowed to stand at room temperature for 1.5 hours. Then A-136 (292.3 μL, 10 mM) that was in an amount 12 times the mole number of the antibody and dissolved in dimethyl sulfoxide solution was added, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-A-136). The DAR value determined by mass spectrometry was 7.91.

### 34. Preparation of Trastuzumab-A-146

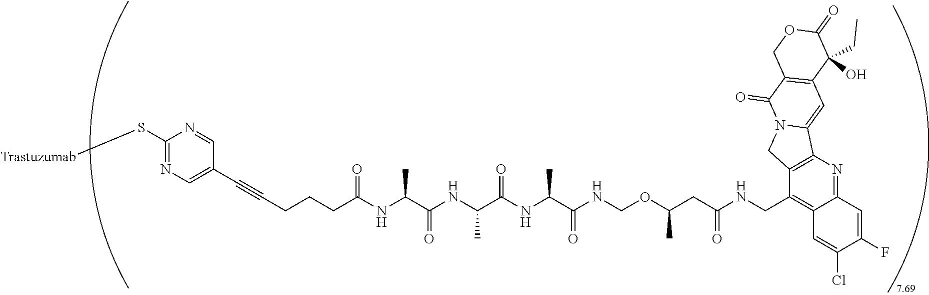

2.333 mL of Trastuzumab (15 mg/mL) was taken, diluted with 117 μL of 20 mM PB+0.1M EDTA (pH 7.60), then adjusted to pH 7.60 with 1M Na₂HPO₄ solution, added with 10 mM TCEP (tri(2-carboxyethyl)phosphine, 132.6 μL, pH 7.60) solution, mixed well, and allowed to stand at room temperature for 1.5 hours. Then A-146 (246 μL, 10 mM) that was in an amount 10 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-A-146). The DAR value determined by mass spectrometry was 7.69.

### 35. Preparation of Trastuzumab-A-151

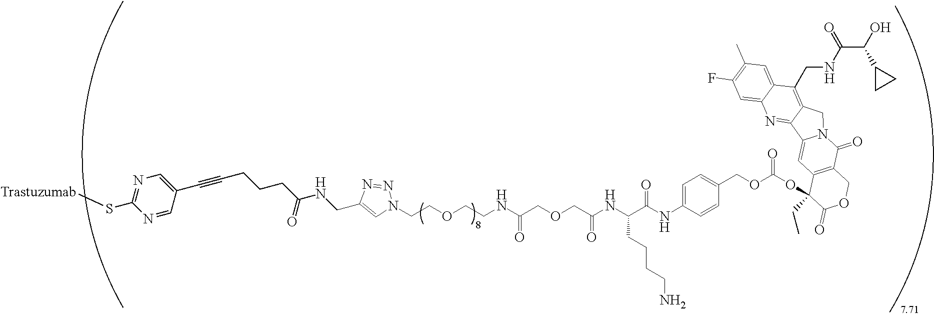

2.041 mL of Trastuzumab (14.7 mg/mL) was taken, diluted with 102 μL of 20 mM PB+0.1M EDTA (pH 7.60), then adjusted to pH 7.6 with 1M Na₂HPO₄ solution, added with 10 mM TCEP (tri(2-carboxyethyl)phosphine, 113.7 μL, pH 7.60) solution, mixed well, and allowed to stand at room temperature for 1.5 hours. Then A-151 (278.7 μL, 10 mM) that was in an amount 12 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-A-151). The DAR value determined by mass spectrometry was 7.71.

### 36. Preparation of Trastuzumab-A-156 (DAR 8)

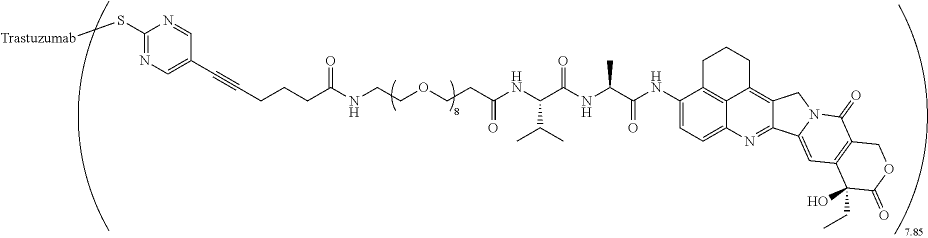

2.041 mL of Trastuzumab (14.7 mg/mL) was taken, dilute with 102 μL of 20 mM PB+0.1 M EDTA (pH 7.60), then adjusted to pH 7.6 with 1 M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then A-156 (207.3 μL, 10 mM) that was in an amount 10 times the mole number of the antibody and dissolved in dimethyl sulfoxide solution was added, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-A-156). The DAR value determined by mass spectrometry was 7.85.

### 37. Preparation of Trastuzumab-A-157 (DAR 8)

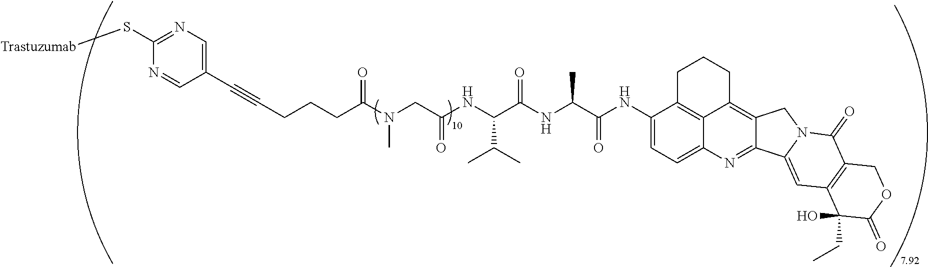

2.041 mL of Trastuzumab (14.7 mg/mL) was taken, diluted with 102 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then A-157 (234.6 μL, 10 mM) that was in an amount 11 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-A-157). The DAR value determined by mass spectrometry was 7.92.

### 38. Preparation of Trastuzumab-A-160 (DAR 8)

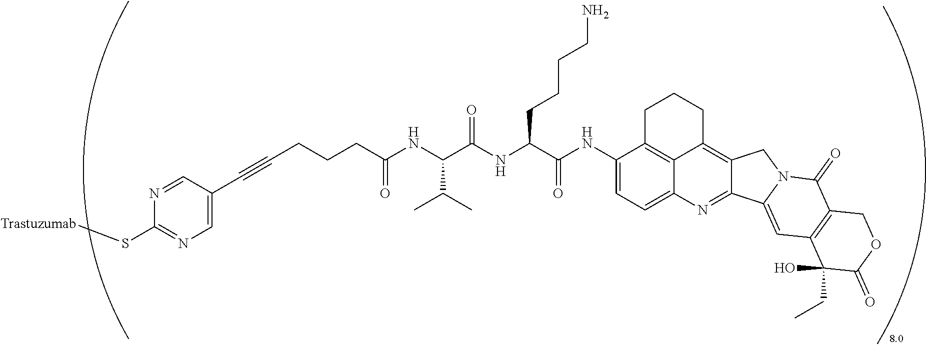

1.75 mL of Trastuzumab (14.7 mg/mL) was taken, diluted with 88 μL of 20 mM PB+0.1 M EDTA (pH 7.60), then adjusted to pH 7.6 with 1 M Na₂HPO₄ solution, added with 15 mM TCEP (tris(2-carboxyethyl)phosphine, 65 μL, pH 7.60) solution, mixed well, and allowed to stand at room temperature for 1.5 hours. Then A-160 (179 μL, 10 mM) that was in an amount 10 times the mole number of the antibody and dissolved in dimethyl sulfoxide solution was added, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-A-160). The DAR value determined by mass spectrometry was 8.0.

### 39. Preparation of Trastuzumab-A-161 (DAR 8)

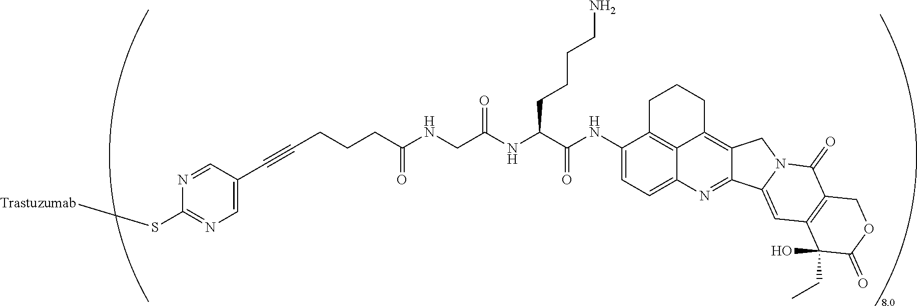

1.75 mL of Trastuzumab (14.7 mg/mL) was taken, diluted with 88 μL of 20 mM PB+0.1 M EDTA (pH 7.60), then adjusted to pH 7.6 with 1 M Na₂HPO₄ solution, added with 15 mM TCEP (tris(2-carboxyethyl)phosphine, 65 μL, pH 7.60) solution, mixed well, and allowed to stand at room temperature for 1.5 hours. Then A-161 (179 μL, 10 mM) that was in an amount 10 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-A-161). The DAR value determined by mass spectrometry was 8.0.

### 40. Preparation of Trastuzumab-A-163 (DAR 8)

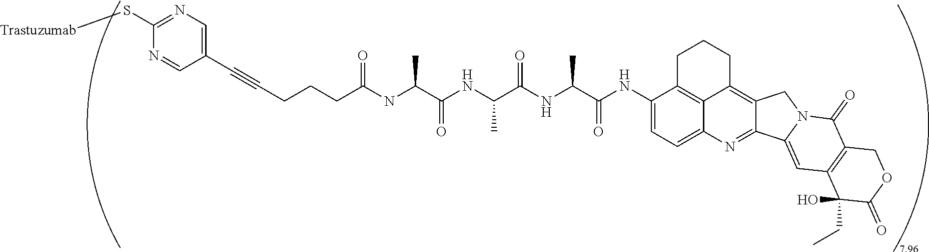

2.041 mL of Trastuzumab (14.7 mg/mL) was taken, diluted with 102 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then A-163 (217.6 μL, 10 mM) that was in an amount 10 times the mole number of the antibody and dissolved in dimethyl sulfoxide solution was added, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-A-163). The DAR value determined by mass spectrometry was 7.96.

### 41. Preparation of Trastuzumab-A-164 (DAR 8)

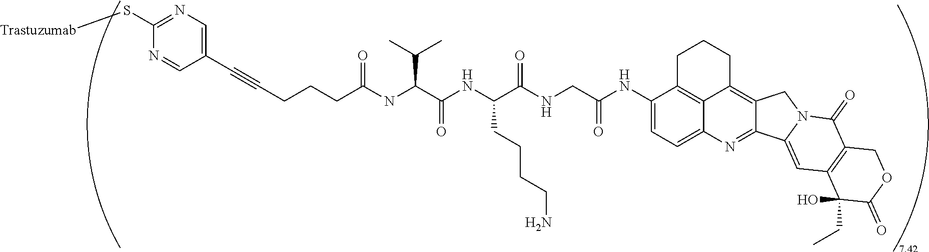

0.238 mL of Trastuzumab (14.7 mg/mL) was taken, diluted with 12 μL of 20 mM PB+0.1 M EDTA (pH 7.60), then adjusted to pH 7.6 with 1 M Na₂HPO₄ solution, added with 10 mM TCEP (tris(2-carboxyethyl)phosphine, 13 μL, pH 7.60) solution, mixed well, and allowed to stand at room temperature for 1.5 hours. Then A-164 (24.36 μL, 10 mM) that was in an amount 10 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-A-164). The DAR value determined by mass spectrometry was 7.42.

### 42. Preparation of Trastuzumab-A-167 (DAR 8)

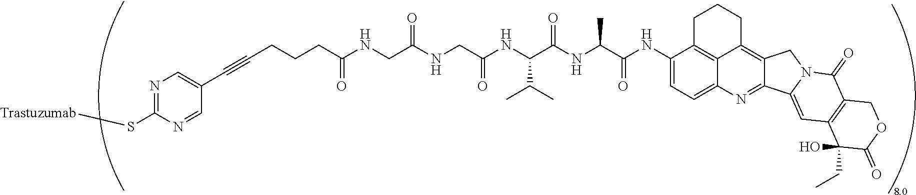

2.333 mL of Trastuzumab (15 mg/mL) was taken, diluted with 117 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 132.6 μL, pH 7.60) solution, mixed well, and allowed to stand at room temperature for 1.5 hours. Then, A-167 (241.6 μL, 10 mM) that was in an amount 10 times the mole number of the antibody and dissolved in dimethyl sulfoxide solution was added, mixed well, and allowed to stand at room temperature for 18 hours. After the above operations were completed, the buffer was replaced with a 20 mM histidine buffer solution at pH 6.0 using NAP gel column (Cytiva) to obtain an antibody-drug conjugate (i.e., Trastuzumab-A-167). The DAR value determined by mass spectrometry was 8.0.

### 43. Preparation of Trastuzumab-A-201 (DAR 8)

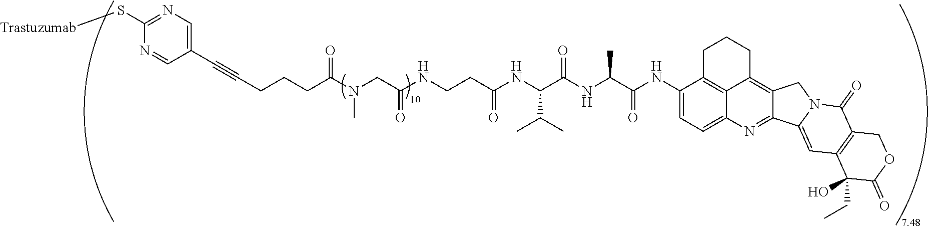

2.041 mL of Trastuzumab (14.7 mg/mL) was taken, diluted with 102 μL of 20 mM PB+0.1 M EDTA (pH 7.60), then adjusted to pH 7.6 with 1 M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then A-201 (208.5 μL, 10 mM) that was in an amount 10 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-A-201). The DAR value determined by mass spectrometry was 7.48.

### 44. Preparation of Trastuzumab-B-3

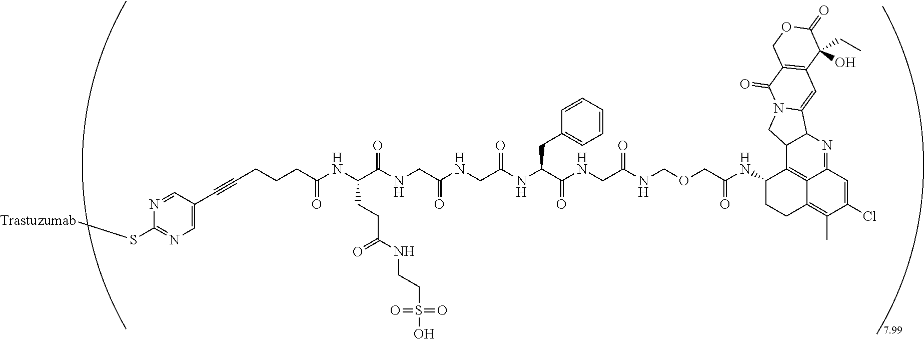

1.234 mL of Trastuzumab antibody (16.2 mg/mL) was taken, diluted with 61.73 μL of 20 mM PB+0.1M EDTA (pH 7.60), then adjusted to pH 7.60 with 1M Na₂HPO₄ solution, added with 10 mM TCEP (tri(2-carboxyethyl)phosphine, 75.8 μL, pH 7.60) solution, mixed well, and allowed to stand at room temperature for 1.5 hours. Then B-3 (145 μL, 10 mM) that was in an amount 10 times the mole number of the antibody and dissolved in dimethyl sulfoxide solution was added, mixed well, and allowed to stand at room temperature for 2 hours. After the above operations were completed, NAP-5 gel column (Cytiva) was used to replace the buffer with 20 mM histidine buffer solution at pH 6.0 to obtain an antibody-drug conjugate (i.e., Trastuzumab-B-3). The DAR value determined by mass spectrometry was 7.99.

### 45. Preparation of Trastuzumab-B-8

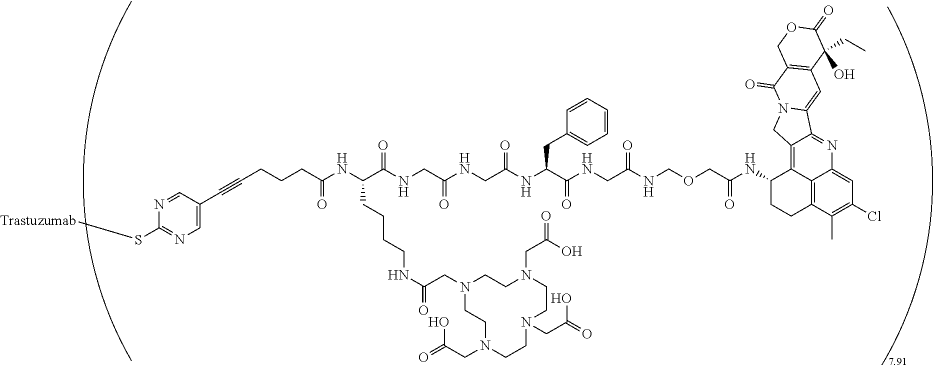

2.041 mL of Trastuzumab (14.7 mg/mL) was taken, diluted with 102 μL of 20 mM PB+0.1M EDTA (pH 7.60), then adjusted to pH 8.0 with 1M Na₂HPO₄ solution, added with 10 mM TCEP (tri(2-carboxyethyl)phosphine, 113.7 μL, pH 7.60) solution, mixed well, and allowed to stand at room temperature for 1.5 hours. Then B-8 (249 μL, 10 mM) that was in an amount 12 times the mole number of the antibody and dissolved in dimethyl sulfoxide solution was added, mixed well, allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-B-8). The DAR value determined by mass spectrometry was 7.91.

### 46. Preparation of Trastuzumab-B-13 (DAR 8)

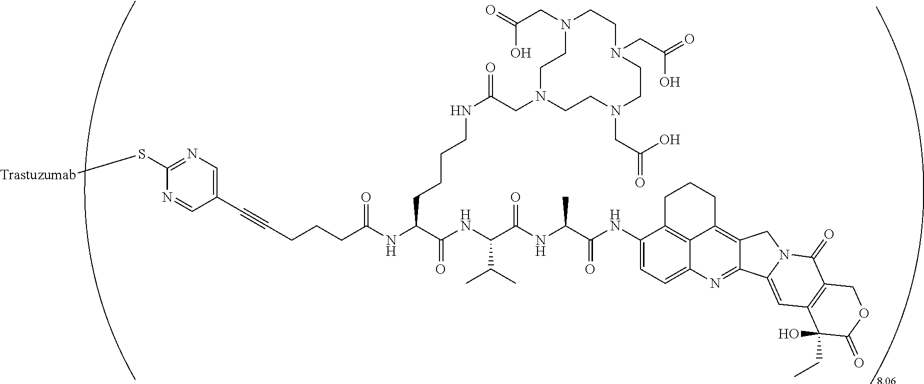

1.156 mL of Trastuzumab (14.7 mg/mL) was taken, diluted with 58 μL of 20 mM PB+0.1 M EDTA (pH 7.60), then adjusted to pH 7.6 with 1 M Na₂HPO₄ solution, added with 10 mM TCEP (tris(2-carboxyethyl)phosphine, 64 μL, pH 7.60) solution, mixed well, and allowed to stand at room temperature for 1.5 hours. Then B-13 (335 μL, 10 mM) that was in an amount 20 times the mole number of the antibody and dissolved in dimethyl sulfoxide solution was added, mixed well, and allowed to stand at room temperature for 18 hours. After the above operations were completed, NAP gel column (Cytiva) was used to replace the buffer with 20 mM histidine buffer solution at pH 6.0 to obtain an antibody-drug conjugate (i.e., Trastuzumab-B-13). The DAR value determined by mass spectrometry was 8.06.

### 47. Preparation of Trastuzumab-B-14 (DAR 8)

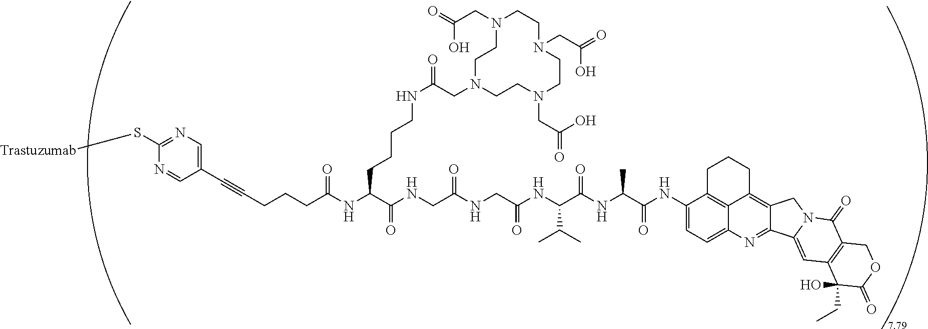

1.36 mL of Trastuzumab (14.7 mg/mL) was taken, diluted with 68 μL of 20 mM PB+0.1 M EDTA (pH 7.60), then adjusted to pH 7.6 with 1 M Na₂HPO₄ solution, added with 10 mM TCEP (tris(2-carboxyethyl)phosphine, 76 μL, pH 7.60) solution, mixed well, and allowed to stand at room temperature for 1.5 hours. Then B-14 (239 μL, 10 mM) that was in an amount 12 times the mole number of the antibody and dissolved in dimethyl sulfoxide solution was added, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-B-14). The DAR value determined by mass spectrometry was 7.79.

### 48. Preparation of Trastuzumab-B-15

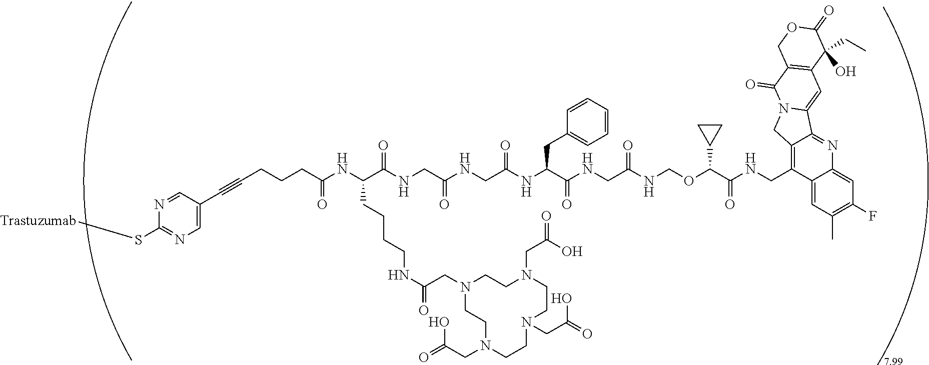

2.041 mL of Trastuzumab (14.7 mg/mL) was taken, diluted with 102 μL of 20 mM PB+0.1M EDTA (pH 7.60), then adjusted to pH 7.6 with 1M Na₂HPO₄ solution, added with 10 mM TCEP (tris (2-carboxyethyl) phosphine, 113.7 μL, pH 7.60) solution, mixed well, and allowed to stand at room temperature for 1.5 hours. Then B-15 (261 μL, 10 mM) that was in an amount 12 times the mole number of the antibody and dissolved in dimethyl sulfoxide solution, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-B-15). The DAR value determined by mass spectrometry was 7.99.

### 49. Preparation of Trastuzumab-A-154

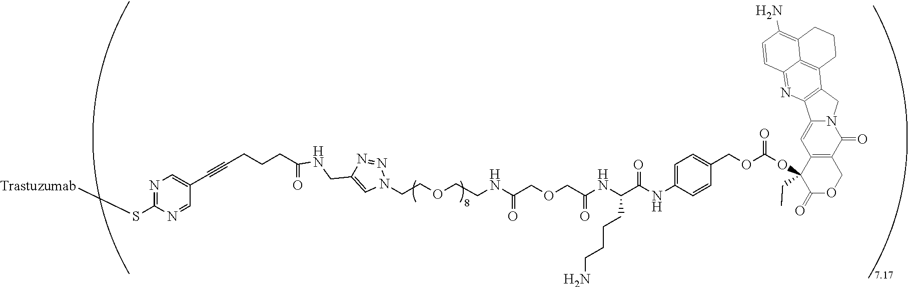

2.041 mL of Trastuzumab (14.7 mg/mL) was taken, diluted with 102 μL of 20 mM PB+0.1M EDTA (pH 7.60), then adjusted to pH 7.60 with 1M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then A-154 (211 μL, 10 mM) that was in an amount 10 times the mole number of the antibody and dissolved in dimethyl sulfoxide solution, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-A-154). The DAR value determined by mass spectrometry was 7.17.

### 50. Preparation of Trastuzumab-A-155

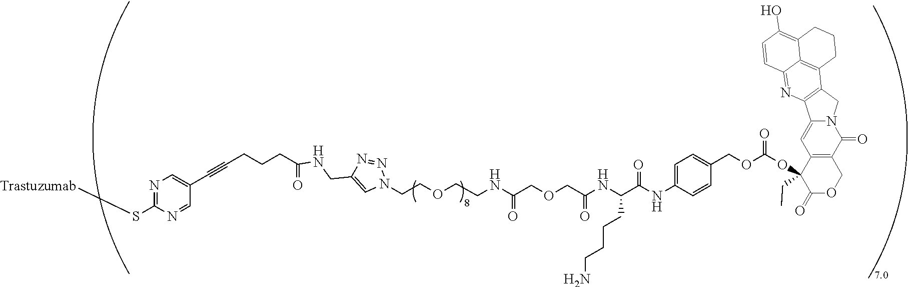

2.041 mL of Trastuzumab (14.7 mg/mL) was taken, diluted with 102 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then A-155 (298.6 μL, 10 mM) that was in an amount 13 times the mole number of the antibody and dissolved in dimethyl sulfoxide solution was added, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-A-155). The DAR value determined by mass spectrometry was 7.0.

### 51. Preparation of Trastuzumab-A-187

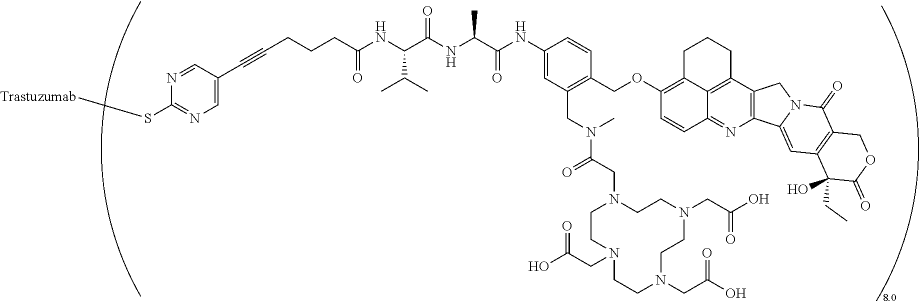

2.027 mL of Trastuzumab (14.8 mg/mL) was taken, diluted with 101 μL of 20 mM PB+0.1M EDTA (pH 7.60), then adjusted to pH 7.60 with 1M Na₂HPO₄ solution, added with 10 mM TCEP (tris(2-carboxyethyl)phosphine, 113.6 μL, pH 7.60) solution, mixed well, and allowed to stand at room temperature for 1.5 hours. Then A-187 (261 μL, 10 mM) that was in an amount 12 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-A-187). The DAR value determined by mass spectrometry was 8.0.

### 52. Preparation of Trastuzumab-B-21

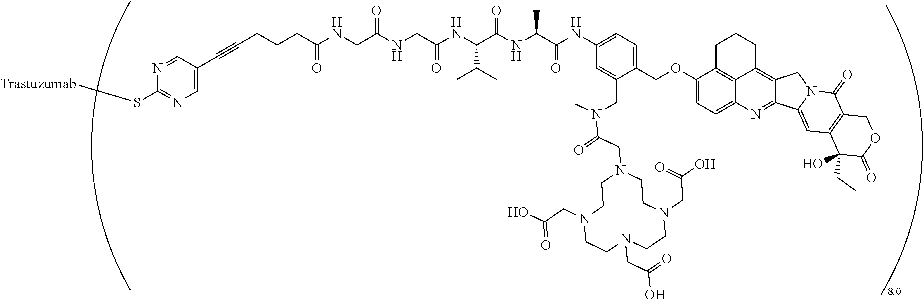

2.027 mL of Trastuzumab (14.8 mg/mL) was taken, diluted with 101 μL of 20 mM PB+0.1M EDTA (pH 7.60), then adjusted to pH 7.60 with 1M Na₂HPO₄ solution, added with 10 mM TCEP (tri(2-carboxyethyl)phosphine, 113.6 μL, pH 7.60) solution, mixed well, and allowed to stand at room temperature for 1.5 hours. Then B-21 (261 μL, 10 mM) that was in an amount 12 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-B-21). The DAR value determined by mass spectrometry was 8.0.

### 53. Preparation of Trastuzumab-B-22

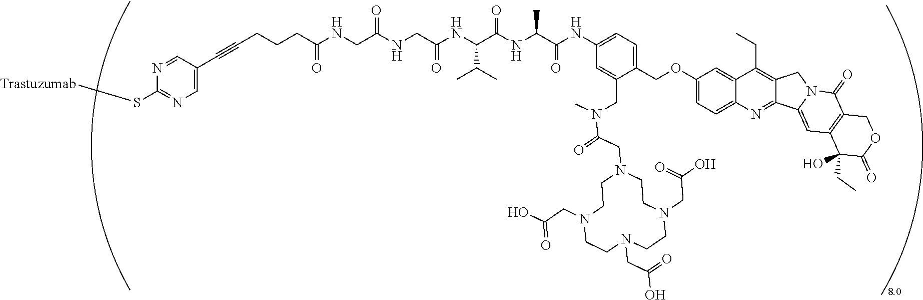

2.027 mL of Trastuzumab (14.8 mg/mL) was taken, diluted with 101 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 113.6 μL, pH 7.60) solution, mixed well, and allowed to stand at room temperature for 1.5 hours. Then B-22 (215.8 μL, 10 mM) that was in an amount 12 times the mole number of the antibody and dissolved in dimethyl sulfoxide solution was added, mixed well, and allowed to stand at room temperature for 2 hours. After the above operations were completed, NAP gel column (Cytiva) was used to replace the buffer with 20 mM histidine buffer solution at pH 6.0 to obtain an antibody-drug conjugate (i.e., Trastuzumab-B-22). The DAR value determined by mass spectrometry was 8.0.

### 54. Preparation of Trastuzumab-B-23

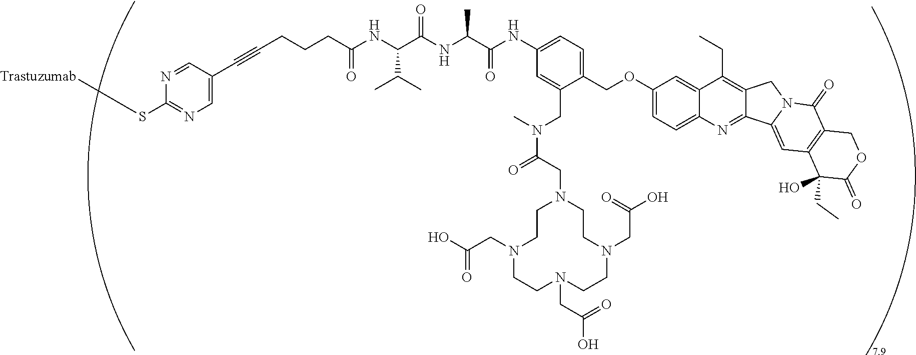

2.027 mL of Trastuzumab (14.8 mg/mL) was taken, diluted with 101 μL of 20 mM PB+0.1M EDTA (pH 7.60), then adjusted to pH 7.60 with 1M Na₂HPO₄ solution, added with 10 mM TCEP (tri(2-carboxyethyl)phosphine, 113.6 μL, pH 7.60) solution, mixed well, and allowed to stand at room temperature for 1.5 hours. Then B-23 (261 μL, 10 mM) that was in an amount 12 times the mole number of the antibody and dissolved in dimethyl sulfoxide solution was added, mixed well, allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-B-23). The DAR value determined by mass spectrometry was 7.9.

### 55. Preparation of Trastuzumab-B-17

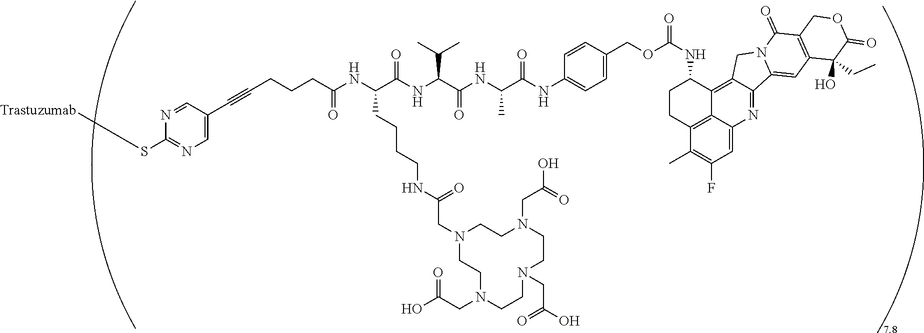

1.7 mL of Trastuzumab (14.7 mg/mL) was taken, diluted with 85 μL of 20 mM PB+0.1 M EDTA (pH 7.60), then adjusted to pH 7.60 with 1 M Na₂HPO₄ solution, added with 10 mM TCEP (tris(2-carboxyethyl)phosphine, 95 μL, pH 7.60) solution, mixed well, and allowed to stand at room temperature for 1.5 hours. Then B-17 (208.8 μL, 10 mM) that was in an amount 12 times the mole number of the antibody and dissolved in dimethyl sulfoxide solution was added, mixed well, allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-B-17). The DAR value determined by mass spectrometry was 7.8.

### 56. Preparation of Trastuzumab-B-20

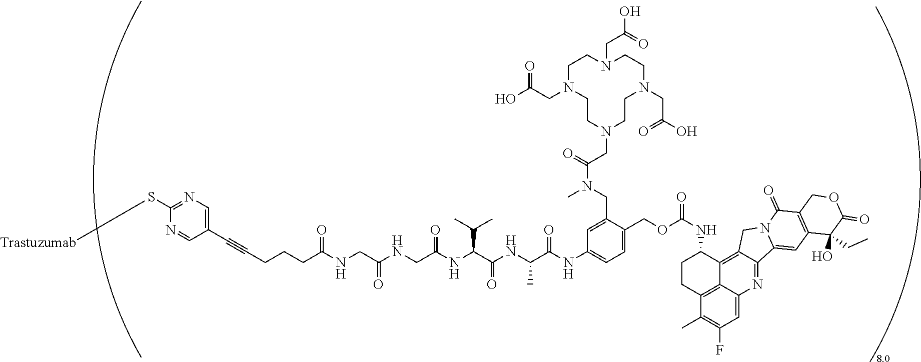

2.027 mL of Trastuzumab (14.8 mg/mL) was taken, diluted with 101 μL of 20 mM PB+0.1M EDTA (pH 7.60), then adjusted to pH 7.60 with 1M Na₂HPO₄ solution, added with 10 mM TCEP (tri(2-carboxyethyl)phosphine, 113.6 μL, pH 7.60) solution, mixed well, and allowed to stand at room temperature for 1.5 hours. Then B-20 (250.5 μL, 10 mM) that was in an amount 12 times the mole number of the antibody and dissolved in dimethyl sulfoxide solution was added, mixed well, and allowed to stand at room temperature for 2 hours. Then NAP gel column (Cytiva) was used to replace the buffer with 20 mM histidine buffer solution at pH 6.0 to obtain an antibody-drug conjugate (i.e., Trastuzumab-B-20). The DAR value determined by mass spectrometry was 8.0.

### IV. Evaluation of Tumor Growth Inhibitory Effects of Antibody-Drug Conjugates in Mouse Subcutaneous Transplanted Tumor Model

### 1. N87 Mouse CDX Model

The preparations containing the ADCs of the present invention were respectively administered through tail vein injection to the mouse CDX model subcutaneously transplanted with human gastric cancer cell NCI-N87, the changes in tumor volume and animal body weight were measured twice weekly, and the tumor inhibition efficacy of the ADCs of the present invention on tumor-bearing mice was calculated.

### Test Drug

Drug name, source, preparation method: The ADCs of the present invention at appropriate amounts were taken; based on the administration volume of 1 mg/kg, physiological saline was used to dilute the stock solution to obtain the administration solution. Physiological saline was used as the solvent control (Vehicle).

### Experimental Animal and Cell Line

Balb/c Nude mice (Chengdu Yaokang Biotechnology Co., Ltd.)

Human gastric cancer cells NCI-N87 (ATCC)

### Experimental Grouping and Evaluation Method

The tumor-bearing mice with tumors having an average volume of 100 to 150 mm³ were selected, and randomly divided into groups (the number of groups was determined according to the number of samples). According to the groups, physiological saline (hereinafter referred to as solvent control, Vehicle) and the ADCs of the present invention were respectively administered. The frequencies of administration were shown in the specific examples. The administration model was tail vein injection, and the administration volume was 10 ml/kg. After administration, the tumor diameters were measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

The tumor growth inhibition rate TGI (%) was calculated using the following formula to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ] * 100 \% or {V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADC of the present invention:

$$T / C = ( {V}_{T end} / {V}_{T 0} ) / ( {V}_{C end} / {V}_{C 0} ) .$$

### (1) Detection of Efficacy of Anti-Human Her2 Antibody-Drug Conjugates in NCI-N87 Model

NCI-N87 cells were cultured in RPMI 1640 medium containing 10% fetal bovine serum at 37° C. and 5% C02. NCI-N87 cells in the exponential growth phase were collected, resuspended in PBS to a suitable concentration, and inoculated subcutaneously in female Balb/c-Nude mice to establish a gastric cancer model. When the average tumor volume was about 160 mm³, the mice were randomly divided into groups according to the tumor size, including in order: a vehicle control group (i.e., negative control, Vehicle group), and a Trastuzumab-A-2 at 1 mg/kg group, a Trastuzumab-A-14 at 1 mg/kg group, a Trastuzumab-A-26 at 1 mg/kg group, a Trastuzumab-A-44 at 1 mg/kg group, a Trastuzumab-A-52 at 1 mg/kg group, a Trastuzumab-A-54 at 1 mg/kg group, a Trastuzumab-A-62 at 1 mg/kg group, and a Trastuzumab-A-76 at 1 mg/kg group of the present invention. Each group adopted tail vein injection (i.v.), and administration was performed on Day 0, Day 7 and Day 14, three times in total. After administration, the long diameter and short diameter of tumor was measured with a vernier caliper twice a week, and the tumor volume was calculated according to the following calculation formula: V=0.5 a×b² wherein a and b represented the long diameter and short diameter of the tumor, respectively. The death of animals was observed and recorded every day.

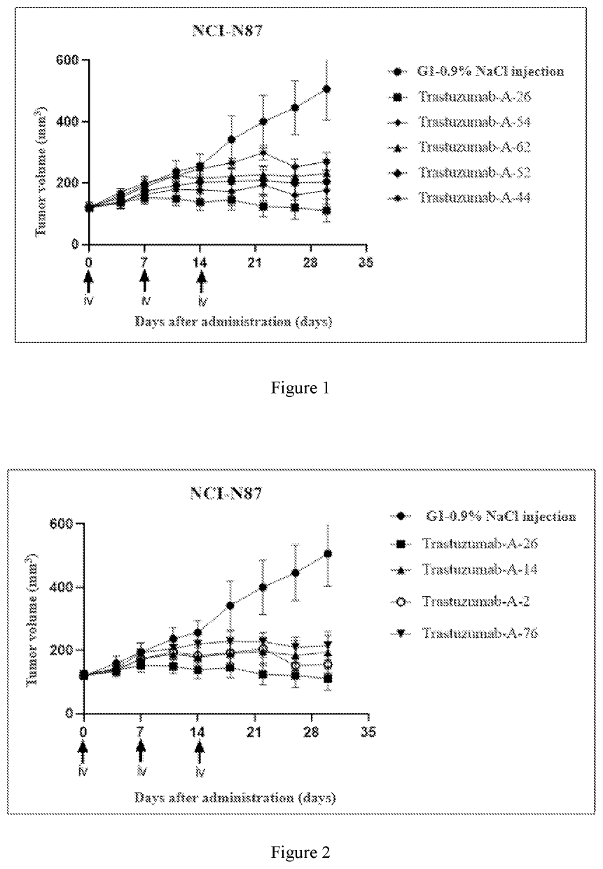

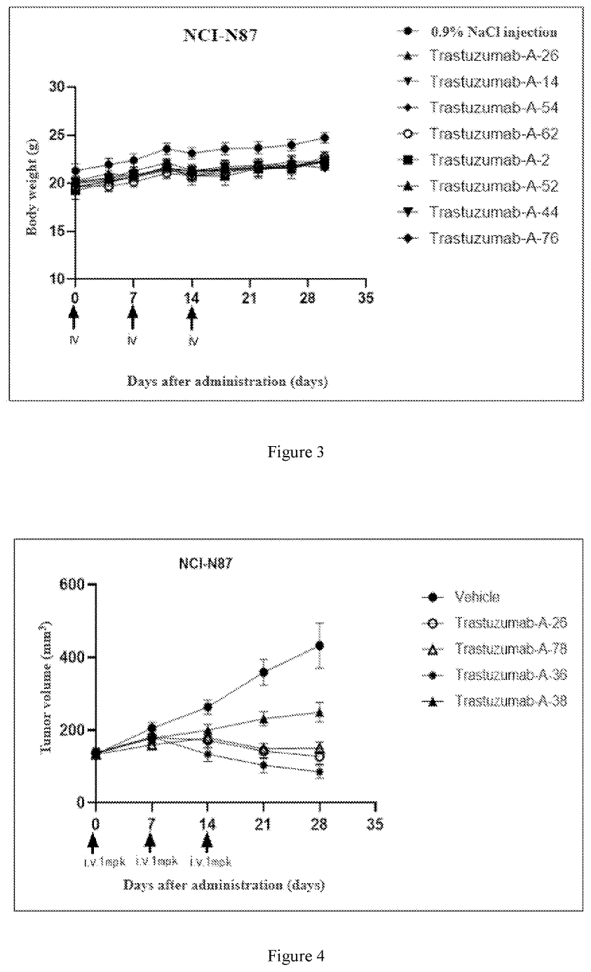

The ADCs of the present invention exhibited significant inhibitory effects on the tumor growth in the NCI-N87 gastric cancer xenograft model. Compared with the Vehicle group, the tumor growth inhibition rate (TGI) values of the Trastuzumab-A-2 at 1 mg/kg group, Trastuzumab-A-14 at 1 mg/kg group, Trastuzumab-A-26 at 1 mg/kg group, Trastuzumab-A-44 at 1 mg/kg group, Trastuzumab-A-52 at 1 mg/kg group, Trastuzumab-A-54 at 1 mg/kg group, Trastuzumab-A-62 at 1 mg/kg group, and Trastuzumab-A-76 at 1 mg/kg group of the present invention were 90.83%, 81.62%, 107.38%, 61.43%, 78.38%, 85.97%, 71.92% and 75.91%, respectively. Until Day 30, there was no animal death and significant animal weight loss in the treatment groups, and no obvious drug toxicity reaction was observed. The mice had good tolerance to the ADCs of the present invention during the treatment period. The specific results are shown in Table 19, and FIGS. 1, 2 and 3.

TABLE 19
Human gastric cancer cell NCI-N87 CDX model

| | | Day 30 | | | |
|---|---|---|---|---|---|
| | | Tumor | | | P value |
| | Dose | volume | | T/ | (vs. |
| | (mg/ | (mm³) | TGI | C | Vehi- |
| Group | kg) | (x̅ ± SEM) | (%) | (%) | cle) |
| Vehicle control | — | 506.05 ± 102.02 | — | — | — |
| Trastuzumab-A-2 | 1 | 156.42 ± 27.02 | 90.83 | 30.59 | <0.01 |
| Trastuzumab-A-14 | 1 | 194.42 ± 53.15 | 81.62 | 37.27 | <0.05 |
| Trastuzumab-A-26 | 1 | 111.44 ± 37.23 | 107.38 | 21.91 | <0.01 |
| Trastuzumab-A-44 | 1 | 270.12 ± 29.44 | 61.43 | 52.76 | <0.05 |
| Trastuzumab-A-52 | 1 | 204.81 ± 72.89 | 78.38 | 39.95 | <0.05 |
| Trastuzumab-A-54 | 1 | 176.72 ± 64.48 | 85.97 | 34.13 | <0.05 |
| Trastuzumab-A-62 | 1 | 232.49 ± 34.23 | 71.92 | 44.35 | <0.05 |
| Trastuzumab-A-76 | 1 | 215.39 ± 43.95 | 75.91 | 41.66 | <0.05 |

Note:
TGI is the tumor growth inhibition rate, and T/C is the relative tumor proliferation rate.

### (2) Detection of Efficacy of Anti-Human Her2 Antibody-Drug Conjugates in NCI-N87 Model

NCI-N87 cells were cultured in RPMI 1640 medium containing 10% fetal bovine serum at 37° C. and 5% CO₂. NCI-N87 cells in the exponential growth phase were collected, resuspended in PBS to a suitable concentration, and inoculated subcutaneously in female Balb/c-Nude mice to establish a gastric cancer model. When the average tumor volume was about 160 mm³, the mice were randomly divided into groups according to the tumor size, including in order: a vehicle control group (i.e., negative control, Vehicle group), and a Trastuzumab-A-26 at 1 mg/kg group, a Trastuzumab-A-78 at 1 mg/kg group, a Trastuzumab-A-36 at 1 mg/kg group, a Trastuzumab-A-38 at 1 mg/kg group, a Trastuzumab-A-72 at 1 mg/kg group, a Trastuzumab-A-70 at 1 mg/kg group, a Pertuzumab-A-26 at 1 mg/kg group, and a Pertuzumab-A-2 at 1 mg/kg group of the present invention. Each group adopted tail vein injection (i.v.), and administration was performed on Day 0, Day 7 and Day 14, three times in total. After administration, the long diameter and short diameter of tumor was measured with a vernier caliper twice a week, and the tumor volume was calculated according to the following calculation formula: V=0.5 a×b² wherein a and b represented the long diameter and short diameter of the tumor, respectively. The death of animals was observed and recorded every day.

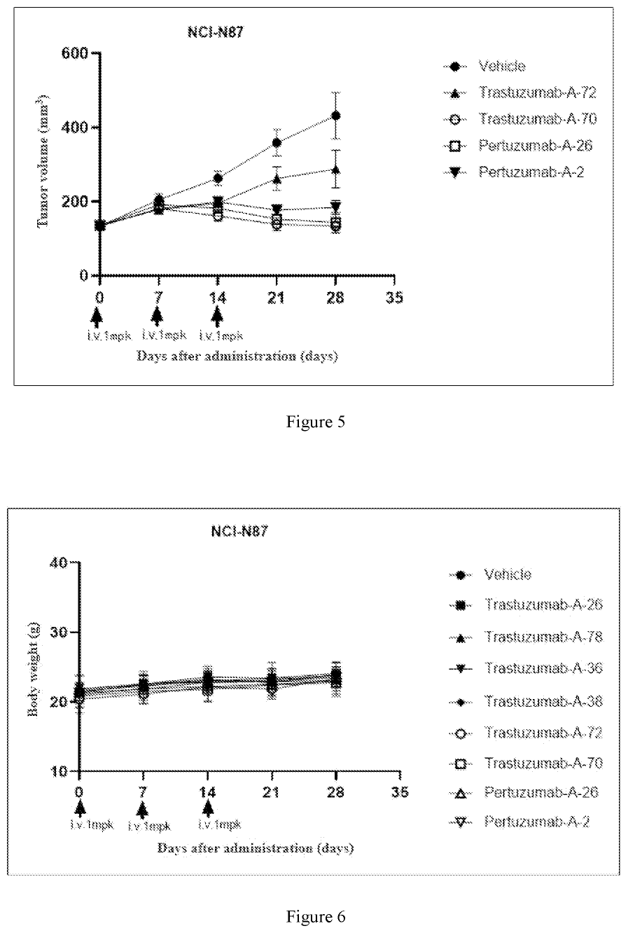

The ADCs of the present invention exhibited significant inhibitory effects on the tumor growth in the NCI-N87 gastric cancer xenograft model. Compared with the Vehicle group, the tumor growth inhibition rate (TGI) values of the Trastuzumab-A-26 at 1 mg/kg group, Trastuzumab-A-78 at 1 mg/kg group, Trastuzumab-A-36 at 1 mg/kg group, Trastuzumab-A-38 at 1 mg/kg group, Trastuzumab-A-72 at 1 mg/kg group, Trastuzumab-A-70 at 1 mg/kg group, Pertuzumab-A-26 at 1 mg/kg group, and Pertuzumab-A-2 at 1 mg/kg group of the present invention were 107.88%, 94.3%, 138.05%, 61.76%, 49.16%, 100.21%, 97.54%, and 83.43%, respectively. Until Day 28, there was no animal death and significant animal weight loss in the treatment groups, and no obvious drug toxicity reaction was observed. The mice had good tolerance to the ADCs of the present invention during the treatment period. The specific results are shown in Table 20, and FIGS. 4, 5 and 6.

TABLE 20
Human gastric cancer cell NCI-N87 CDX model

| | | Day 28 | | | |
|---|---|---|---|---|---|
| | | Tumor | | | P value |
| | | volume | | | (vs. |
| | Dose | (mm³) | TGI | T/C | Vehi |
| Group | (mg/kg) | (x̅ ± SEM) | (%) | (%) | cle) |
| Vehicle | — | 432.08 ± 62.23 | — | — | — |
| Trastuzumab-A-26 | 1 | 127.58 ± 21.39 | 107.88 | 28.67 | <0.01 |
| Trastuzumab-A-78 | 1 | 150.92 ± 16.43 | 94.30 | 35.07 | <0.01 |
| Trastuzumab-A-36 | 1 | 85.49 ± 17.80 | 138.05 | 19.28 | <0.01 |
| Trastuzumab-A-38 | 1 | 249.29 ± 27.01 | 61.76 | 57.26 | <0.05 |
| Trastuzumab-A-70 | 1 | 134.01 ± 19.00 | 100.21 | 31.06 | <0.01 |
| Pertuzumab-A-26 | 1 | 143.66 ± 26.71 | 97.54 | 32.80 | <0.01 |
| Pertuzumab-A-2 | 1 | 184.28 ± 18.48 | 83.43 | 42.50 | <0.01 |

Note:
TGI is the tumor growth inhibition rate, and T/C is the relative tumor proliferation rate, the same below.

### (3) Detection of Efficacy of Anti-Human Her2 Antibody-Drug Conjugates in NCI-N87 Model

NCI-N87 cells were cultured in RPMI 1640 medium containing 10% fetal bovine serum at 37° C. and 5% C02. NCI-N87 cells in the exponential growth phase were collected, resuspended in PBS to a suitable concentration, and inoculated subcutaneously in female Balb/c-Nude mice to establish a gastric cancer model. When the average tumor volume was about 150 mm³, the mice were randomly divided into groups according to the tumor size, including in order: a vehicle control group (i.e., negative control, Vehicle group), and a Trastuzumab-A-26 at 1 mg/kg group, and a Trastuzumab-B-3 at 1 mg/kg group of the present invention. Each group adopted tail vein injection (i.v.), and administration was performed on Day 0, Day 7 and Day 14, three times in total. After administration, the long diameter and short diameter of tumor was measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

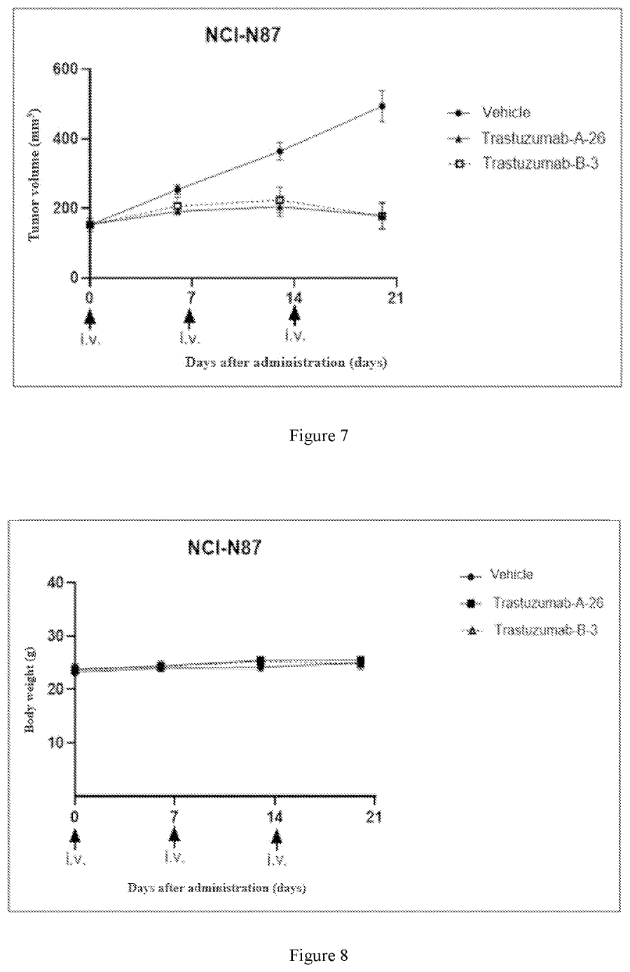

The ADCs of the present invention exhibited significant inhibitory effects on the tumor growth in the NCI-N87 gastric cancer xenograft model. Compared with the Vehicle group, the tumor growth inhibition rate (TGI) values of the Trastuzumab-A-26 at 1 mg/kg group, Trastuzumab-B-3 at 1 mg/kg group of the present invention were 92.40%, 92.69%, respectively. Until Day 20, there was no animal death and significant animal weight loss in the treatment groups, and no obvious drug toxicity reaction was observed. The mice had good tolerance to the ADCs of the present invention during the treatment period. The specific results are shown in Table 21, and FIGS. 7 and 8.

TABLE 21
Human gastric cancer cell NCI-N87 CDX model

| | | Day 20 | | | |
|---|---|---|---|---|---|
| | | Tumor | | | |
| | | volume | | | P value |
| | Dose | (mm³) | TGI | T/C | (vs. |
| Group | (mg/kg) | x̅ ± (SEM) | (%) | (%) | Vehicle) |
| Vehicle | — | 494.44 ± 44.48 | — | — | — |
| Trastuzumab-A-26 | 1 | 179.18 ± 38.78 | 92.40 | 36.02 | <0.01 |
| Trastuzumab-B-3 | 1 | 177.59 ± 37.98 | 92.69 | 35.84 | <0.01 |

Note:
TGI is the tumor growth inhibition rate, and T/C is the relative tumor proliferation rate, the same below.

### 2. JIMT-1 Mouse CDX Model

The preparations containing the ADCs of the present invention were administered via tail vein injection to the mouse CDX model subcutaneously transplanted with human breast cancer cell JIMT-1. The changes in tumor volume and animal body weight were measured twice weekly, and the tumor inhibition efficacy of the ADCs of the present invention on tumor-bearing mice was calculated.

### Test Drug

Drug name, source, preparation method: The ADCs of the present invention at appropriate amounts were taken; based on the administration volume of 3 mg/kg, 0.9% NaCl injection was used to dilute the stock solution to form the administration solution. 0.9% NaCl injection was used as the solvent control (Vehicle).

### Experimental Animal and Cell Line

NOD SCID mice (Chengdu Yaokang Biotechnology Co., Ltd.)

Human breast cancer cells JIMT-1 (Nanjing Kebai)

### Experimental Grouping and Evaluation Method

Tumor-bearing mice with an average tumor volume of about 150 mm³ were selected and randomly divided into groups (the number of groups was determined according to the number of samples). According to the groups, 0.9% NaCl injection (hereinafter referred to as solvent control, Vehicle) and the ADCs of the present invention were administered, respectively. The administration frequencies were shown in the specific examples. The administration model was tail vein injection, and the administration volume was 10 ml/kg. After administration, the tumor diameters were measured with a vernier caliper twice a week, and the tumor volume was calculated according to the following calculation formula: V=0.5 a×b², where a and b represent the long diameter and short diameter of the tumor, respectively. Animal deaths were observed and recorded every day.

The following formula was used to calculate the tumor growth inhibition rate TGI (%), which was used to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ] * 100 \% or {V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADCs of the present invention:

$$T / C = ( {V}_{T end} / {V}_{T 0} ) / ( {V}_{C end} / {V}_{C 0} ) .$$

### Detection of Efficacy of Anti-Human Her2 Antibody-Drug Conjugate in JIMT-1 Model

JIMT-1 cells were cultured in DMEM medium containing 10% fetal bovine serum at 37° C. and 5% CO₂. JIMT-1 cells in the exponential growth phase were collected, resuspended in PBS containing 50% matrigel to a suitable concentration, and inoculated subcutaneously in female NOD SCID mice to establish a breast cancer model. When the average tumor volume was about 150 mm³, the mice were randomly divided into groups according to the tumor size, including in order: a vehicle control group (i.e., negative control, Vehicle group), and a Trastuzumab-A-26 at 3 mg/kg group, a Trastuzumab-A-14 at 3 mg/kg group, a Trastuzumab-A-54 at 3 mg/kg group, and a Trastuzumab-A-2 at 3 mg/kg group of the present invention. Each group adopted tail vein injection (i.v.), and administration was performed on Day 0, Day 7 and Day 14, three times in total. After administration, the long diameter and short diameter of tumor was measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

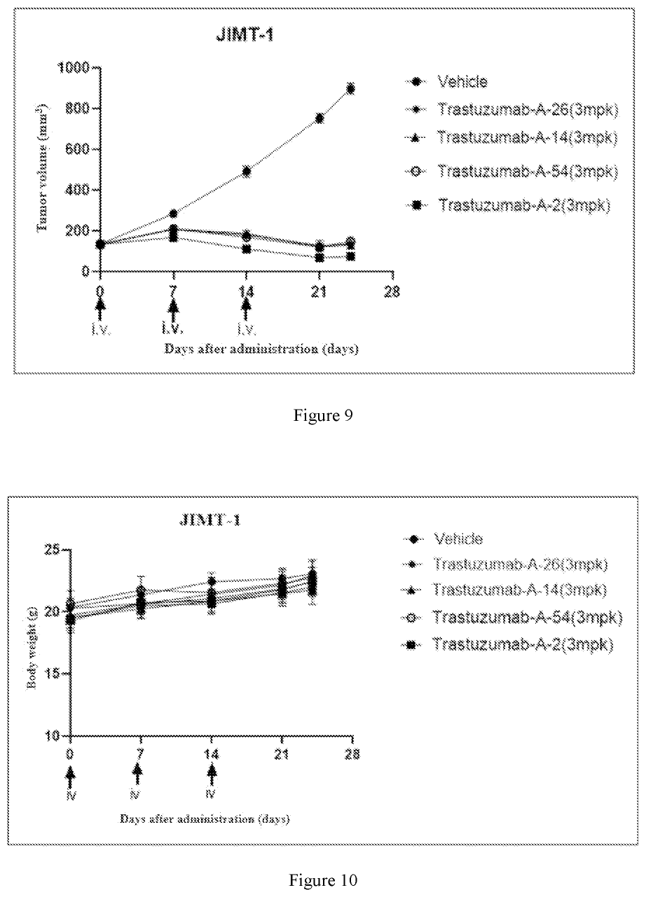

The ADCs of the present invention exhibited significant inhibitory effects on the tumor growth in the JIMT-1 breast cancer xenograft model. Compared with the Vehicle group, the tumor growth inhibition rate (TGI) values of the Trastuzumab-A-26 at 3 mg/kg group, Trastuzumab-A-14 at 3 mg/kg group, Trastuzumab-A-54 at 3 mg/kg group, and Trastuzumab-A-2 at 3 mg/kg group of the present invention were 100.61%, 102.25%, 98.20%, 143.33%, respectively. Until Day 24, there was no animal death and significant animal weight loss in the treatment groups, and no obvious drug toxicity reaction was observed. The mice had good tolerance to the ADCs of the present invention during the treatment period. The specific results are shown in Table 22, FIGS. 9 and 10.

TABLE 22
Human breast cancer cell JIMT-1 CDX model

| | | Day 24 | | | |
|---|---|---|---|---|---|
| | | Tumor | | | P value |
| | | volume | | | (vs. |
| | Dose | (mm³) | TGI | T/C | Vehi- |
| Group | (mg/kg) | (x̅ ± SEM) | (%) | (%) | cle) |
| Vehicle | — | 898.76 ± 25.73 | — | — | — |
| Trastuzumab-A-26 | 3 | 135.04 ± 24.70 | 100.61 | 15.02 | <0.001 |
| Trastuzumab-A-14 | 3 | 131.31 ± 8.11 | 102.25 | 14.78 | <0.001 |
| Trastuzumab-A-54 | 3 | 74.41 ± 6.51 | 98.20 | 16.66 | <0.001 |
| Trastuzumab-A-2 | 3 | 148.75 ± 6.76 | 143.33 | 8.57 | <0.001 |

Note:
TGI is the tumor growth inhibition rate, T/C is the relative tumor proliferation rate, the same below.

### 3. HCC827 Mouse CDX Model

The preparations containing the ADCs of the present invention were administered via tail vein injection to the mouse CDX model subcutaneously transplanted with human non-small cell lung cancer cell HCC827. The changes in tumor volume and animal body weight were measured twice weekly, and the tumor inhibition efficacy of the ADCs of the present invention on tumor-bearing mice was calculated.

### Test Drug

Drug name, source, preparation method: The ADCs of the present invention at appropriate amounts were taken; based on the administration volume of 10 mg/kg, 0.9% NaCl injection was used to dilute the stock solution to form the administration solution. 0.9% NaCl injection was used as the solvent control (Vehicle).

### Experimental Animal and Cell Line

Balb/c-Nude mice (Chengdu Yaokang Biotechnology Co., Ltd.)

Human non-small cell lung cancer cells HCC827 (Nanjing Kebai)

### Experimental Grouping and Evaluation Method

Tumor-bearing mice with an average tumor volume of about 150 mm³ were selected and randomly divided into groups (the number of groups was determined according to the number of samples). According to the groups, 0.9% NaCl injection (hereinafter referred to as solvent control, Vehicle) and the ADCs of the present invention were administered, respectively. The administration frequencies were shown in the specific examples. The administration model was tail vein injection, and the administration volume was 10 ml/kg. After administration, the tumor diameters were measured with a vernier caliper twice a week, and the tumor volume was calculated according to the following calculation formula: V=0.5 a×b², where a and b represent the long diameter and short diameter of the tumor, respectively. Animal deaths were observed and recorded every day.

The following formula was used to calculate the tumor growth inhibition rate TGI (%), which was used to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ] * 100 \% or {V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADCs of the present invention:

$$T / C = ( {V}_{T end} / {V}_{T 0} ) / ( {V}_{C end} / {V}_{C 0} ) .$$

### Detection of Efficacy of Anti-Human Her2 Antibody-Drug Conjugate in HCC827 Model

HCC827 cells were cultured in RPMI-1640 medium containing 10% fetal bovine serum at 37° C. and 5% C02. HCC827 cells in the exponential growth phase were collected, resuspended in PBS containing 50% matrigel to a suitable concentration, and inoculated subcutaneously in female Balb/c-Nude mice to establish a human non-small cell lung cancer model. When the average tumor volume was about 150 mm³, the mice were randomly divided into groups according to the tumor size, including in order: a vehicle control group (i.e., negative control, Vehicle group), and a Trastuzumab-A-14 at 10 mg/kg group, a Trastuzumab-A-26 at 10 mg/kg group, a Trastuzumab-A-1 at 10 mg/kg group, and a Trastuzumab-A-2 at 10 mg/kg group of the present invention. Each group adopted tail vein injection (i.v.), and administration was performed on Day 0 and Day 7, twice in total. After administration, the long diameter and short diameter of tumor was measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

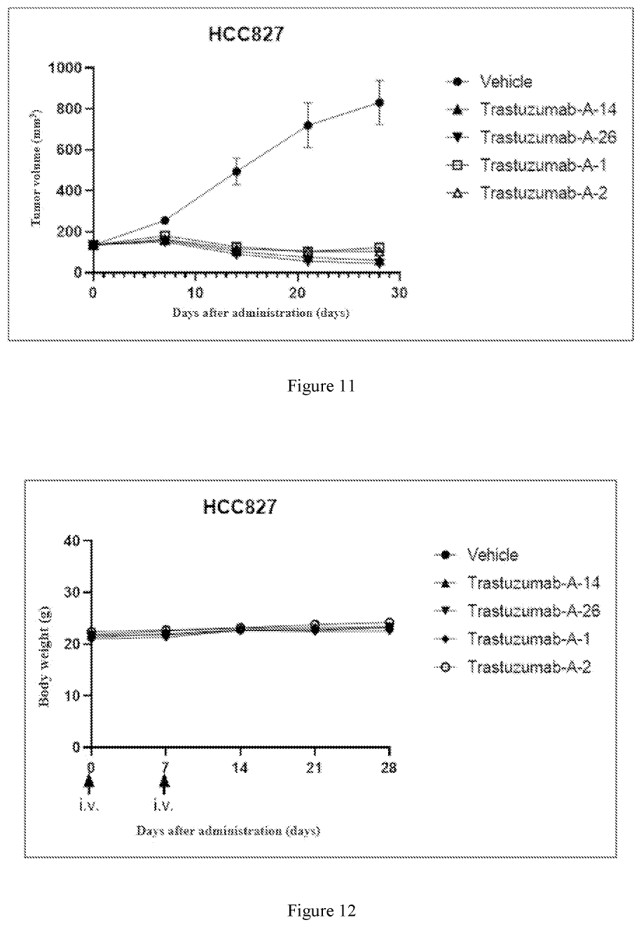

The ADCs of the present invention exhibited significant inhibitory effects on the tumor growth in the HCC827 human non-small cell lung cancer xenograft model. Compared with the Vehicle group, the tumor growth inhibition rate (TGI) values of the Trastuzumab-A-14 at 10 mg/kg group, Trastuzumab-A-26 at 10 mg/kg group, Trastuzumab-A-1 at 10 mg/kg group, and Trastuzumab-A-2 at 10 mg/kg group of the present invention were 155.15%, 165.95%, 109.93%, 122.66%, respectively. Until Day 28, there was no animal death and significant animal weight loss in the treatment groups, and no obvious drug toxicity reaction was observed. The mice had good tolerance to the ADCs of the present invention during the treatment period. The specific results are shown in Table 23, FIGS. 11 and 12.

TABLE 23
Human non-small cell lung cancer cell HCC827 CDX model

| | | Day 28 | | | |
|---|---|---|---|---|---|
| | | Tumor | | | P value |
| | Dose | volume | | T/ | (vs. |
| | (mg/ | (mm³) | TGI | C | Vehi- |
| Group | kg) | (x̅ ± SEM) | (%) | (%) | cle) |
| Vehicle | — | 830.93 ± 108.99 | — | — | — |
| Trastuzumab-A-14 | 10 | 60.26 ± 7.75 | 155.15 | 7.28 | <0.001 |
| Trastuzumab-A-26 | 10 | 45.73 ± 6.69 | 165.95 | 5.53 | <0.001 |
| Trastuzumab-A-1 | 10 | 121.92 ± 22.87 | 109.93 | 14.62 | <0.001 |
| Trastuzumab-A-2 | 10 | 104.25 ± 9.05 | 122.66 | 12.56 | <0.001 |

Note:
TGI is the tumor growth inhibition rate, T/C is the relative tumor proliferation rate, the same below.

### V. Evaluation of Tumor Growth Inhibitory Effects of Antibody-Drug Conjugates in Mouse Subcutaneous Transplanted Tumor Model

The preparations containing the ADCs of the present invention were respectively administered through tail vein injection to the mouse CDX model subcutaneously transplanted with human gastric cancer cell NCI-N87, the changes in tumor volume and animal body weight were measured twice weekly, and the tumor inhibition efficacy of the ADCs of the present invention on tumor-bearing mice was calculated.

### Test Drug

Drug name, source, preparation method: The ADCs of the present invention (Sichuan Kelun-Biotech Biopharmaceutical Co., Ltd.) at appropriate amounts were taken; based on the administration volume of 1 mg/kg, 0.9% NaCl injection was used to dilute the stock solution to obtain the administration solution. 0.9% NaCl injection was used as the solvent control (Vehicle).

### Experimental Animal and Cell Line

Balb/c Nude mice (Chengdu Yaokang Biotechnology Co., Ltd., production license number: SCXK (Sichuan) 2020-0034, animal certificate number: 511214900025102)

Human gastric cancer cells NCI-N87 (ATCC)

### Experimental Grouping and Evaluation Method

The tumor-bearing mice with tumors having an average volume of about 150 mm³ were selected, and randomly divided into groups (the number of groups was determined according to the number of samples). According to the groups, 0.9% NaCl injection (hereinafter referred to as solvent control, Vehicle) and the ADCs of the present invention were respectively administered. The frequencies of administration were shown in the specific examples. The administration model was tail vein injection, and the administration volume was 10 ml/kg. After administration, the tumor diameters were measured with a vernier caliper twice a week, and the tumor volume was calculated according to the following calculation formula: V=0.5 a×b² wherein a and b represented the long diameter and short diameter of the tumor, respectively. The death of animals was observed and recorded every day.

The tumor growth inhibition rate TGI (%) was calculated using the following formula to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ] * 100 \% or {V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADC of the present invention:

$$T / C = ( {V}_{T end} / {V}_{T 0} ) / ( {V}_{C end} / {V}_{C 0} ) .$$

### Detection of Efficacy of Anti-Human Her2 Antibody-Drug Conjugates in NCI-N87 Model

NCI-N87 cells were cultured in RPMI 1640 medium containing 10% fetal bovine serum at 37° C. and 5% CO₂. NCI-N87 cells in the exponential growth phase were collected, resuspended in PBS to a suitable concentration, and inoculated subcutaneously in female Balb/c-Nude mice to establish a gastric cancer model. When the average tumor volume was about 150 mm³, the mice were randomly divided into groups according to the tumor size, including in order: a vehicle control group (i.e., negative control, Vehicle group), a Trastuzumab-A-146 at 1 mg/kg group, a Trastuzumab-A-136 at 1 mg/kg group, a Trastuzumab-A-97 at 1 mg/kg group, a Trastuzumab-A-99 at 1 mg/kg group, a Trastuzumab-A-98 at 1 mg/kg group, a Trastuzumab-B-8 at 1 mg/kg group of the present invention, and a control DS8201 at 1 mg/kg group (Note: DS8201 was an ADC targeting human HER2 that was developed by Daiichi Sankyo, and the sample used in this experiment was prepared internally by Kelun-Biotech). Each group adopted tail vein injection (i.v.), and administration was performed on Day 0, Day 7 and Day 14, three times in total. After administration, the long diameter and short diameter of tumor was measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

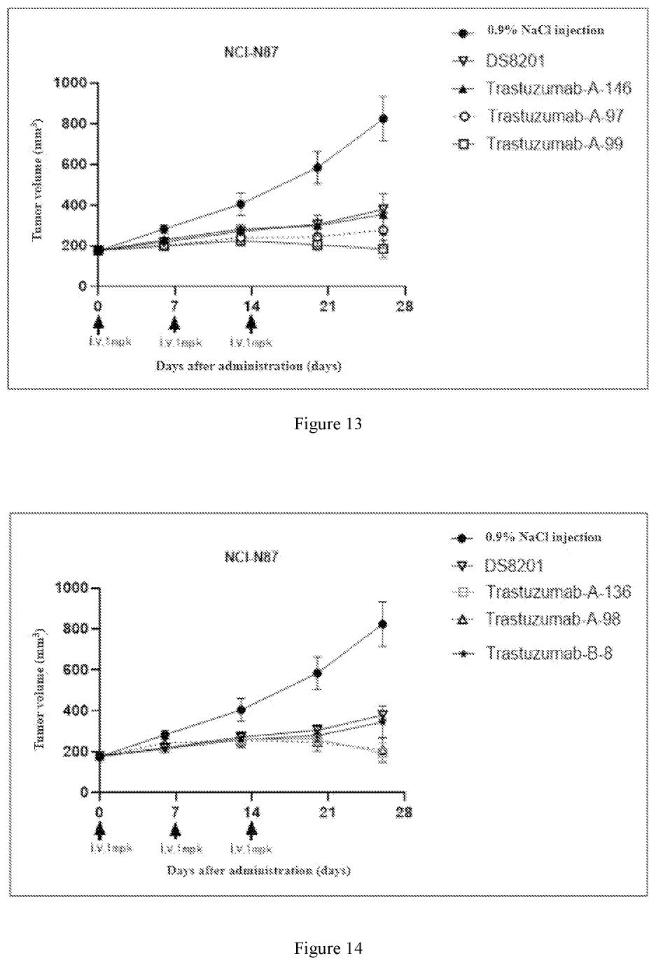

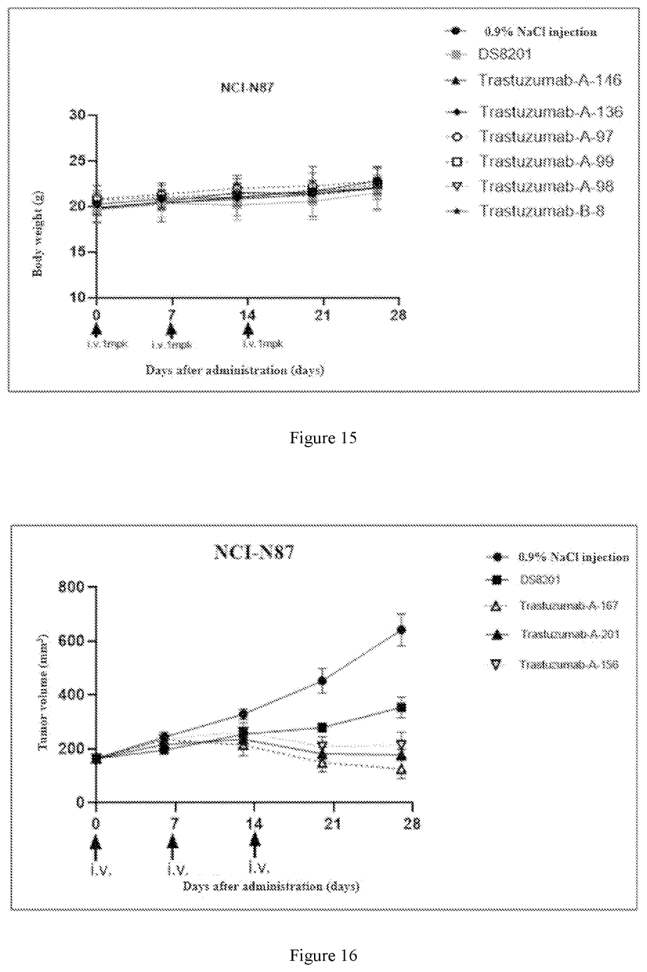

The ADCs of the present invention exhibited significant inhibitory effects on the tumor growth in the NCI-N87 gastric cancer xenograft model. Compared with the Vehicle group, the tumor growth inhibition rate (TGI) values of the Trastuzumab-A-146 at 1 mg/kg group, Trastuzumab-A-136 at 1 mg/kg group, Trastuzumab-A-97 at 1 mg/kg group, Trastuzumab-A-99 at 1 mg/kg group, Trastuzumab-A-98 at 1 mg/kg group, Trastuzumab-B-8 at 1 mg/kg group of the present invention and the control DS8201 at 1 mg/kg group were 72.48%, 97.39%, 84.54%, 99.01%, 95.16%, 74.10%, 69.02%, respectively. Until Day 27, there was no animal death and significant animal weight loss in the treatment groups, and no obvious drug toxicity reaction was observed. The mice had good tolerance to the ADCs of the present invention during the treatment period. The specific results are shown in Table 24, and FIGS. 13, 14 and 15.

TABLE 24
Human gastric cancer cell NCI-N87 CDX model

| | | Day 27 | | | |
|---|---|---|---|---|---|
| | | Tumor | | | P value |
| | | volume | | | (vs. |
| | Dose | (mm³) | TGI | T/C | Vehi- |
| Group | (mg/kg) | (x̅ ± SEM) | (%) | (%) | cle) |
| Vehicle | — | 823.72 ± 108.03 | — | — | — |
| DS8201 (self-made) | 1 | 378.76 ± 21.93 | 69.02 | 45.27 | <0.05 |
| Trastuzumab-A-146 | 1 | 353.55 ± 102.17 | 72.48 | 42.92 | <0.05 |
| Trastuzumab-A-136 | 1 | 193.34 ± 49.06 | 97.39 | 23.29 | <0.05 |
| Trastuzumab-A-97 | 1 | 276.77 ± 88.80 | 84.54 | 33.32 | <0.05 |
| Trastuzumab-A-99 | 1 | 183.81 ± 42.87 | 99.01 | 22.02 | <0.05 |
| Trastuzumab-A-98 | 1 | 209.26 ± 58.13 | 95.16 | 25.00 | <0.05 |
| Trastuzumab-B-8 | 1 | 345.55 ± 77.98 | 74.10 | 41.36 | <0.05 |

Note:
TGI is the tumor growth inhibition rate, and T/C is the relative tumor proliferation rate, the same below.

### VI. Evaluation of Tumor Growth Inhibitory Effects of Antibody-Drug Conjugates in Mouse Subcutaneous Transplanted Tumor Model

The preparations containing the ADCs of the present invention were respectively administered through tail vein injection to the mouse CDX model subcutaneously transplanted with human gastric cancer cell NCI-N87, the changes in tumor volume and animal body weight were measured twice weekly, and the tumor inhibition efficacy of the ADCs of the present invention on tumor-bearing mice was calculated.

### Test Drug

Drug name, source, preparation method: The ADCs of the present invention (Sichuan Kelun-Biotech Biopharmaceutical Co., Ltd.) at appropriate amounts were taken; based on the administration volume, 0.9% NaCl injection was used to dilute the stock solution to obtain the administration solution. 0.9% NaCl injection was used as the solvent control (Vehicle).

### Experimental Animal and Cell Line

Balb/c Nude mice (Chengdu Yaokang Biotechnology Co., Ltd., production license number: SCXK (Sichuan) 2020-0034, animal certificate number: 511214900026661)

Human gastric cancer cells NCI-N87 (ATCC)

### Experimental Grouping and Evaluation Method

The tumor-bearing mice with tumors having an average volume of about 150 mm³ were selected, and randomly divided into groups (the number of groups was determined according to the number of samples). According to the groups, 0.9% NaCl injection (hereinafter referred to as solvent control, Vehicle) and the ADCs of the present invention were respectively administered. The frequencies of administration were shown in the specific examples. The administration model was tail vein injection, and the administration volume was 10 ml/kg. After administration, the tumor diameters were measured with a vernier caliper twice a week, and the tumor volume was calculated according to the following calculation formula: V=0.5 a×b² wherein a and b represented the long diameter and short diameter of the tumor, respectively. The death of animals was observed and recorded every day.

The tumor growth inhibition rate TGI (%) was calculated using the following formula to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ] * 100 \% or {V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADC of the present invention:

$$T / C = ( {V}_{T end} / {V}_{T 0} ) / ( {V}_{C end} / {V}_{C 0} ) .$$

### Detection of Efficacy of Anti-Human Her2 Antibody-Drug Conjugates in NCI-N87 Model

NCI-N87 cells were cultured in RPMI 1640 medium containing 10% fetal bovine serum at 37° C. and 5% CO₂. NCI-N87 cells in the exponential growth phase were collected, resuspended in PBS to a suitable concentration, and inoculated subcutaneously in female Balb/c-Nude mice to establish a gastric cancer model. When the average tumor volume was about 150 mm³, the mice were randomly divided into groups according to the tumor size, including in order: a vehicle control group (i.e., negative control, Vehicle group), a Trastuzumab-A-167 group, a Trastuzumab-A-163 group, a Trastuzumab-A-157 group, a Trastuzumab-A-201 group, a Trastuzumab-A-156 group (administered at 0.5 mg/kg on Day 0, administered at 1 mg/kg on Day 7, administered at 1 mg/kg on Day 14) of the present invention, and a control DS8201 at 1 mg/kg group (Note: DS8201 was an ADC targeting human HER2 that was developed by Daiichi Sankyo, and the sample used in this experiment was prepared internally by Kelun-Biotech). Each group adopted tail vein injection (i.v.), and administration was performed on Day 0, Day 7 and Day 14, three times in total. After administration, the long diameter and short diameter of tumor was measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

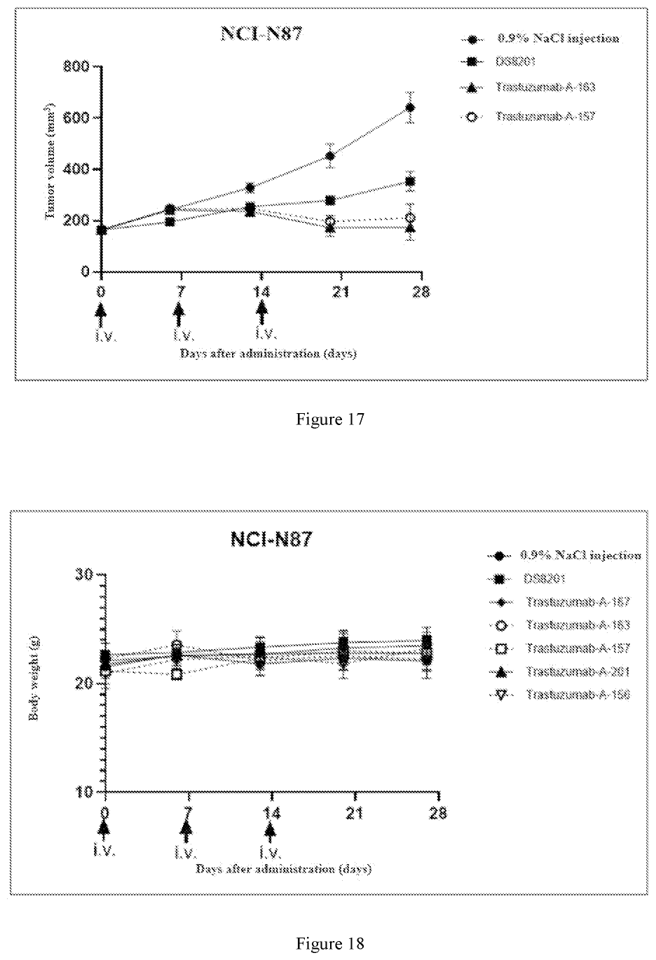

The ADCs of the present invention exhibited significant inhibitory effects on the tumor growth in the NCI-N87 gastric cancer xenograft model. Compared with the Vehicle group, the tumor growth inhibition rate (TGI) values of the Trastuzumab-A-167 group, Trastuzumab-A-163 group, Trastuzumab-A-157 group, Trastuzumab-A-201 group, Trastuzumab-A-156 group (administered at 0.5 mg/kg on Day 0, administered at 1 mg/kg on Day 7, administered at 1 mg/kg on Day 14) of the present invention and the control DS8201 at 1 mg/kg group were 123.19%, 98.12%, 89.97%, 96.96%, 89.14%, 60.21%, respectively. Until Day 27, there was no animal death and significant animal weight loss in the treatment groups, and no obvious drug toxicity reaction was observed. The mice had good tolerance to the ADCs of the present invention during the treatment period. The specific results are shown in Table 25, and FIGS. 16, 17 and 18.

TABLE 25
Human gastric cancer cell NCI-N87 CDX model

| | | Day 27 | | | |
|---|---|---|---|---|---|
| | | Tumor | | | |
| | | volume | | | P value |
| | Dose | (mm³) | TGI | T/C | (vs. |
| Group | (mg/kg) | (x̅ ± SEM) | (%) | (%) | Vehicle) |
| Vehicle | — | 641.88 ± 59.85 | — | — | — |
| DS8201 (self-made) | 1 | 353.383 ± 8.06 | 60.21 | 55.46 | <0.01 |
| Trastuzumab-A-167 | D 0 0.5, D 7/D 14 1 | 125.46 ± 35.40 | 123.19 | 19.71 | <0.001 |
| Trastuzumab-A-163 | D 0 0.5, D 7/D 14 1 | 173.36 ± 50.63 | 98.12 | 27.07 | <0.01 |
| Trastuzumab-A-157 | D 0 0.5, D 7/D 14 1 | 210.96 ± 53.17 | 89.97 | 33.20 | <0.01 |
| Trastuzumab-A-201 | D 0 0.5, D 7/D 14 1 | 178.00 ± 20.20 | 96.96 | 27.94 | <0.001 |
| Trastuzumab-A-156 | D 0 0.5, D 7/D 14 1 | 213.91 ± 48.51 | 89.14 | 33.87 | <0.01 |

Note:
TGI is the tumor growth inhibition rate, and T/C is the relative tumor proliferation rate, the same below.

### VII. Evaluation of Tumor Growth Inhibition Effect of Antibody-Drug Conjugates in Mouse Subcutaneous Transplanted Tumor Model

The preparations containing the ADCs of the present invention were administered via tail vein injection to the mouse CDX model subcutaneously transplanted with human breast cancer cell JIMT-1. The changes in tumor volume and animal body weight were measured twice weekly, and the tumor inhibition efficacy of the ADCs of the present invention on tumor-bearing mice was calculated.

### Test Drug

Drug name, source, preparation method: The ADCs of the present invention (Sichuan Kelun-Biotech Biopharmaceutical Co., Ltd.) at appropriate amounts were taken; administrated at 3 mg/kg, and the specific administration doses were shown below. 0.9% NaCl injection was used to dilute the stock solution to form the administration solution. 0.9% NaCl injection was used as the solvent control (Vehicle).

### Experimental Animal and Cell Line

NOD SCID mice (GemPharmatech Co., Ltd, production license number: SCXK (Su) 2023-0009, animal certificate number: B202402230090)

Human breast cancer cells JIMT-1 (Nanjing Kebai)

### Experimental Grouping and Evaluation Method

Tumor-bearing mice with an average tumor volume of about 150 mm³ were selected and randomly divided into groups (the number of groups was determined according to the number of samples). According to the groups, 0.9% NaCl injection (hereinafter referred to as solvent control, Vehicle) and the ADCs of the present invention were administered, respectively. The administration frequencies were shown in the specific examples. The administration model was tail vein injection, and the administration volume was 10 ml/kg. After administration, the tumor diameters were measured with a vernier caliper twice a week, and the tumor volume was calculated according to the following calculation formula: V=0.5 a×b², where a and b represent the long diameter and short diameter of the tumor, respectively. Animal deaths were observed and recorded every day.

The following formula was used to calculate the tumor growth inhibition rate TGI (%), which was used to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ] * 100 \% or {V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADCs of the present invention:

$$T / C = ( {V}_{T end} / {V}_{T 0} ) / ( {V}_{C end} / {V}_{C 0} ) .$$

### Detection of Efficacy of Anti-Human Her2 Antibody-Drug Conjugates in JIMT-1 Model

JIMT-1 cells were cultured in DMEM medium containing 10% fetal bovine serum at 37° C. and 5% CO₂. JIMT-1 cells in the exponential growth phase were collected, resuspended in PBS containing 50% matrigel to a suitable concentration, and inoculated subcutaneously in female NOD SCID mice to establish a breast cancer model. When the average tumor volume was about 150 mm³, the mice were randomly divided into groups according to the tumor size, including in order: a vehicle control group (i.e., negative control, Vehicle group), and a Trastuzumab-A-160 at 3 mg/kg group, a Trastuzumab-A-167 at 3 mg/kg group, a Trastuzumab-B-13 at 3 mg/kg group, a Trastuzumab-B-14 at 3 mg/kg group, a Trastuzumab-A-154 at 3 mg/kg group, a Trastuzumab-A-155 at 3 mg/kg group of the present invention, and the control DS8201 at 3 mg/kg (Note: DS8201 was an ADC targeting human HER2 that was developed by Daiichi Sankyo, and the sample used in this experiment was prepared internally by Kelun-Biotech). Each group adopted tail vein injection (i.v.), and administration was performed on Day 0, once in total.

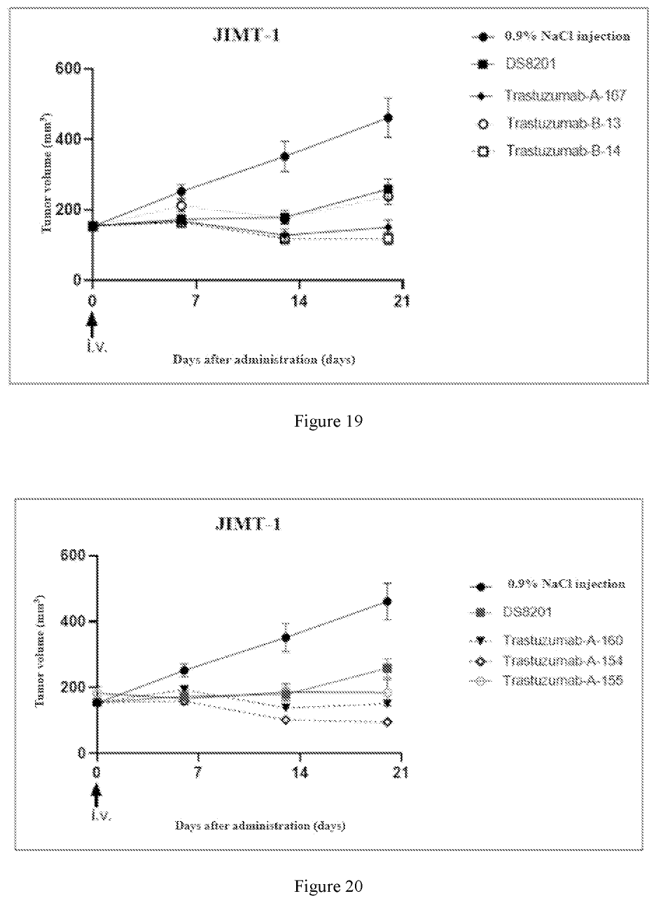

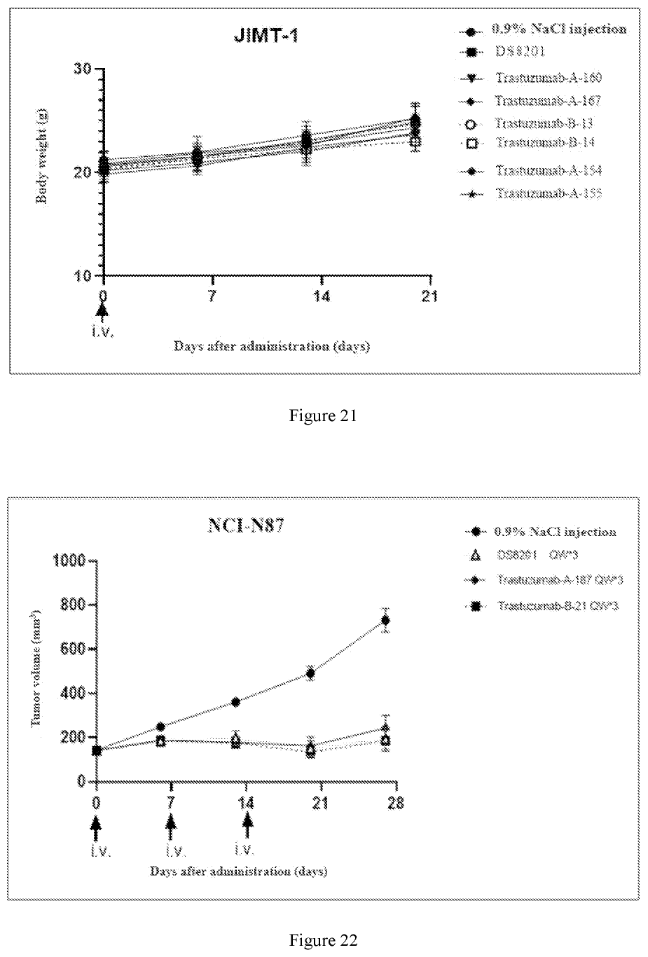

The ADCs of the present invention exhibited significant inhibitory effects on the tumor growth in the JIMT-1 breast cancer xenograft model. Compared with the Vehicle group, the tumor growth inhibition rate (TGI) values of the Trastuzumab-A-160 at 3 mg/kg group, Trastuzumab-A-167 at 3 mg/kg group, Trastuzumab-B-13 at 3 mg/kg group, Trastuzumab-B-14 at 3 mg/kg group, Trastuzumab-A-154 at 3 mg/kg group, Trastuzumab-A-155 at 3 mg/kg group, of the present invention and the control DS8201 at 3 mg/kg group were 102.34%, 103.01%, 72.18%, 122.35%, 138.03%, 72.03% and 66.25%, respectively. Until Day 20, there was no animal death and significant animal weight loss in the treatment groups, and no obvious drug toxicity reaction was observed. The mice had good tolerance to the ADCs of the present invention during the treatment period. The specific results are shown in Table 26, FIGS. 19, 20 and 21.

TABLE 26
Human breast cancer cell JIMT-1 CDX model

| | | Day 20 | | | |
|---|---|---|---|---|---|
| | | Tumor | | | P value |
| | | volume | | | (vs. |
| | Dose | (mm³) | TGI | T/C | Vehi- |
| Group | (mg/kg) | (x̅ ± SEM) | (%) | (%) | cle) |
| Vehicle | — | 461.44 ± 55.93 | — | — | — |
| DS8201 | 3 | 259.25 ± 27.93 | 66.25 | 55.45 | <0.05 |
| Trastuzumab-A-154 | 3 | 95.97 ± 10.71 | 138.03 | 20.57 | <0.001 |
| Trastuzumab-A-155 | 3 | 241.14 ± 24.50 | 72.03 | 51.66 | <0.05 |
| Trastuzumab-A-160 | 3 | 151.30 ± 10.25 | 102.34 | 32.42 | <0.01 |
| Trastuzumab-A-167 | 3 | 150.92 ± 20.58 | 103.01 | 32.20 | <0.01 |
| Trastuzumab-B-13 | 3 | 238.33 ± 22.67 | 72.18 | 51.85 | <0.001 |
| Trastuzumab-B-14 | 3 | 119.49 ± 17.91 | 122.35 | 25.78 | <0.001 |

### VIII. Evaluation of Tumor Growth Inhibitory Effects of Antibody-Drug Conjugates in Mouse Subcutaneous Transplanted Tumor Model

The preparations containing the ADCs of the present invention were respectively administered through tail vein injection to the mouse CDX model subcutaneously transplanted with human gastric cancer cell NCI-N87, the changes in tumor volume and animal body weight were measured twice weekly, and the tumor inhibition efficacy of the ADCs of the present invention on tumor-bearing mice was calculated.

### Test Drug

ADCs at appropriate amounts were taken, and administered based on 1 mg/kg, and the specific administration doses were shown below. 0.9% NaCl injection was used to dilute the stock solution to obtain the administration solution. 0.9% NaCl injection was used as the solvent control (Vehicle).

### Experimental Animal and Cell Line

Balb/c Nude mice (Chengdu Yaokang Biotechnology Co., Ltd., production license number: SCXK (Sichuan) 2020-0034, animal certificate number: 511214900030725)

Human gastric cancer cells NCI-N87 (ATCC)

### Experimental Grouping and Evaluation Method

The tumor-bearing mice with tumors having an average volume of about 150 mm³ were selected, and randomly divided into groups (the number of groups was determined according to the number of samples). According to the groups, 0.9% NaCl injection (hereinafter referred to as solvent control, Vehicle) and the ADCs of the present invention were respectively administered. The frequencies of administration were shown in the specific examples. The administration model was tail vein injection, and the administration volume was 10 ml/kg. After administration, the tumor diameters were measured with a vernier caliper twice a week, and the tumor volume was calculated according to the following calculation formula: V=0.5 a×b² wherein a and b represented the long diameter and short diameter of the tumor, respectively. The death of animals was observed and recorded every day.

The tumor growth inhibition rate TGI (%) was calculated using the following formula to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ] * 100 \% or {V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADC of the present invention:

$$T / C = ( {V}_{T end} / {V}_{T 0} ) / ( {V}_{C end} / {V}_{C 0} ) * 100 \% .$$

### Detection of Efficacy of Anti-Human Her2 Antibody-Drug Conjugates in NCI-N87 Model

NCI-N87 cells were cultured in RPMI 1640 medium containing 10% fetal bovine serum at 37° C. and 5% CO₂. NCI-N87 cells in the exponential growth phase were collected, resuspended in PBS to a suitable concentration, and inoculated subcutaneously in female Balb/c-Nude mice to establish a gastric cancer model. When the average tumor volume was about 150 mm³, the mice were randomly divided into groups according to the tumor size, including in order: a vehicle control group (i.e., negative control, Vehicle group), a Trastuzumab-A-187 at 1 mg/kg group, a Trastuzumab-B-21 at 1 mg/kg group, a Trastuzumab-A-155 at 1 mg/kg group, a Trastuzumab-A-154 at 1 mg/kg group, a Trastuzumab-B-17 at 1 mg/kg group of the present invention, and a control DS8201 at 1 mg/kg group (Note: DS8201 was an ADC targeting human HER2 that was developed by Daiichi Sankyo, and the sample used in this experiment was prepared internally by Kelun-Biotech). Each group adopted tail vein injection (i.v.); Trastuzumab-B-17 was administered on Day 0 and Day 7, twice in total; and the control DS8201 and other ADCs of the present invention were administered on Day 0, Day 7 and Day 14, three times in total.

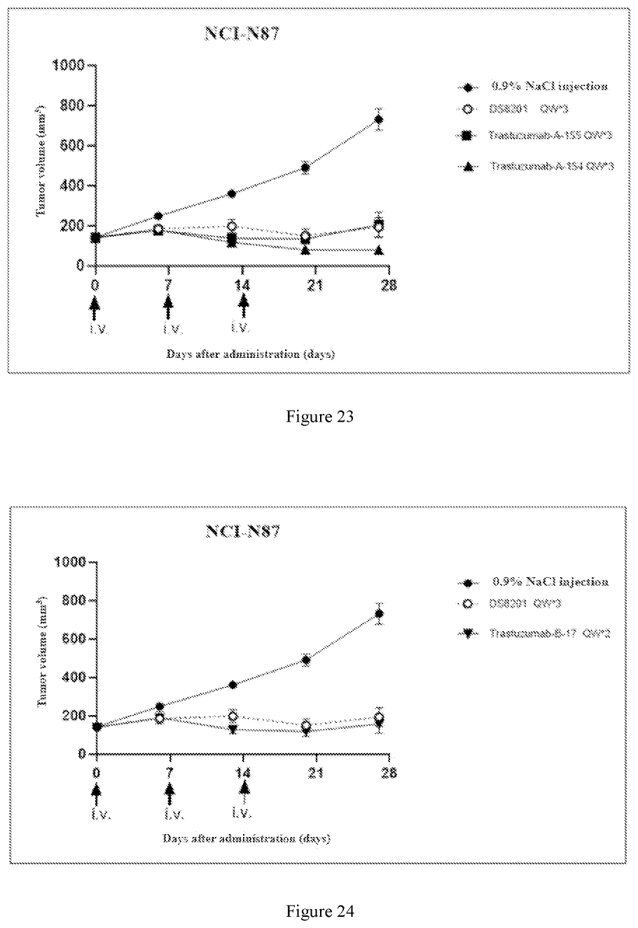

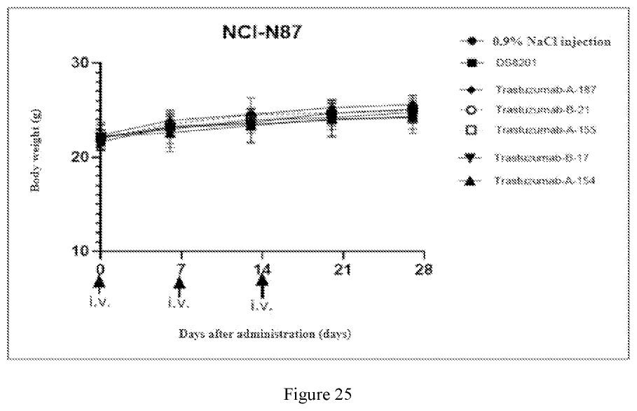

The ADCs of the present invention exhibited significant inhibitory effects on the tumor growth in the NCI-N87 gastric cancer xenograft model. Compared with the Vehicle group, the tumor growth inhibition rate (TGI) values of the Trastuzumab-A-187 group, Trastuzumab-B-21 group, Trastuzumab-A-155 group, Trastuzumab-A-154 group, Trastuzumab-B-17 group of the present invention and the DS8201 group were 82.91%, 92.43%, 89.60%, 143.10%, 96.94% and 91.25%, respectively. Until Day 27, there was no animal death and significant animal weight loss in the treatment groups, and no obvious drug toxicity reaction was observed. The mice had good tolerance to the ADCs of the present invention during the treatment period. The specific results are shown in Table 27, and FIGS. 22, 23, 24 and 25.

TABLE 27
Human gastric cancer cell NCI-N87 CDX model

| | | Day 27 | | | |
|---|---|---|---|---|---|
| | | Tumor | | | P value |
| | | volume | | | (vs. |
| | Dose | (mm³) | TGI | T/C | Vehi- |
| Group | (mg/kg) | (x̅ ± SEM) | (%) | (%) | cle) |
| Vehicle | — | 732.45 ± 53.51 | — | — | — |
| DS8201 | 1 | 194.7 ± 48.78 | 91.25 | 26.66 | <0.001 |
| Trastuzumab-A-187 | 1 | 243.80 ± 58.54 | 82.91 | 33.39 | <0.001 |
| Trastuzumab-B-21 | 1 | 186.25 ± 47.31 | 92.43 | 25.77 | <0.001 |
| Trastuzumab-A-155 | 1 | 205.89 ± 62.52 | 89.60 | 27.90 | <0.001 |
| Trastuzumab-A-154 | 1 | 79.80 ± 17.42 | 143.10 | 11.15 | <0.001 |
| Trastuzumab-B-17 | 1 | 158.29 ± 48.42 | 96.94 | 22.12 | <0.001 |

Although the specific embodiments of the present invention have been described in detail, those skilled in the art will understand that various modifications and substitutions can be made to those details based on all the teachings disclosed, and these changes are within the scope of protection of the present invention. The full scope of the present invention is given by the appended claims and any equivalents thereof.

1. A compound represented by Formula (I) or a pharmaceutically acceptable salt thereof, 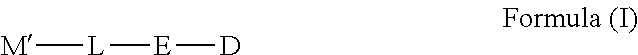
wherein:
M′ represents -M-Lg, wherein Lg is a leaving group for a nucleophilic substitution reaction, and M is a structural fragment capable of binding to a target moiety;
L represents a structural fragment connecting M and E;
E represents a structural fragment connecting L and D;
D represents a cytotoxic drug fragment;
preferably, Lg is selected from the group consisting of halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, hydroxyl (—OH), sulfhydryl group (—SH), amino (—NH₂), nitro, azido, cyano, alkenyl, alkynyl and alkynyl-containing structural fragment, and the halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, alkenyl, alkynyl and alkynyl-containing structural fragment are optionally substituted with one or more suitable substituents;
preferably, Lg is selected from the group consisting of halogen, substituted or unsubstituted C₁₋₆ alkylsulfonyl, halogenated phenoxy, hydroxyl (—OH), sulfhydryl group (—SH) or amino (—NH₂);
preferably, Lg is selected from C₁₋₆ alkylsulfonyl, such as methylsulfonyl.

2. The compound or pharmaceutically acceptable salt thereof according to claim 1, wherein M is selected from the group consisting of the following substituted or unsubstituted structural fragments:

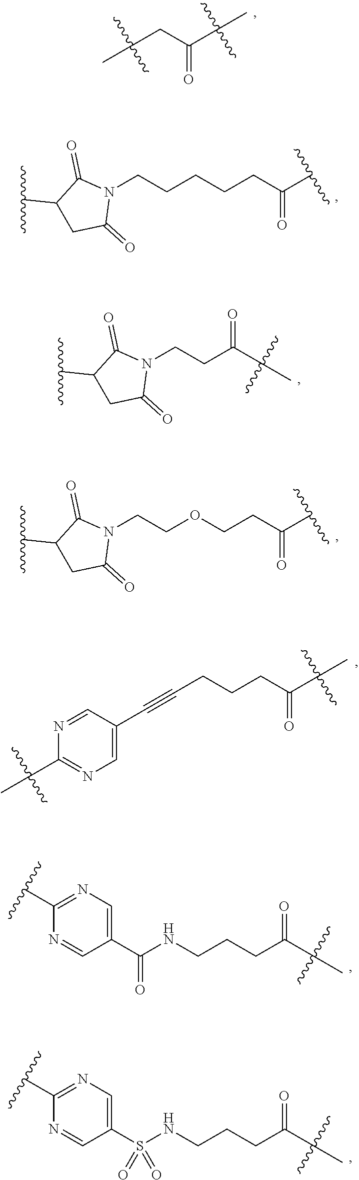

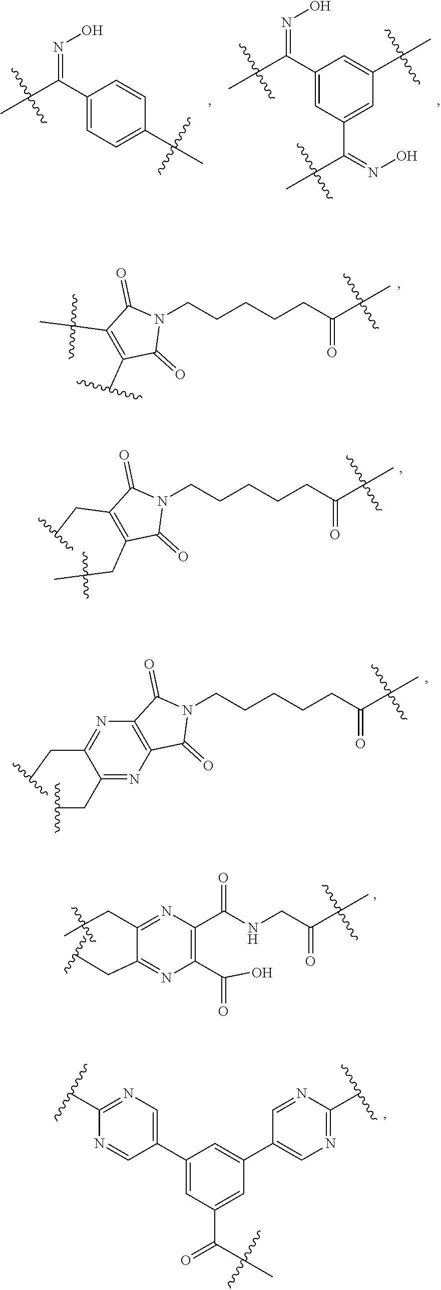

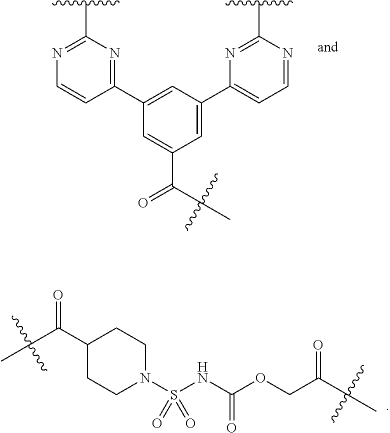 [SMILES: *C(=O)c1cc(-c2ccnc(*)n2)cc(-c2ccnc(*)n2)c1 ; *C(=O)COC(=O)NS(=O)(=O)N1CCC(C(*)=O)CC1]

3. The compound or pharmaceutically acceptable salt thereof according to claim 1, wherein M is selected from the group consisting of the following substituted or unsubstituted structural fragments:

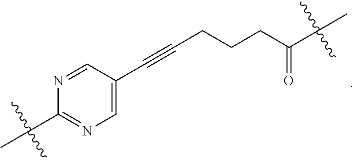 [SMILES: *C(=O)CCCC#Cc1cnc(*)nc1]

4. The compound or pharmaceutically acceptable salt thereof according to claim 1, wherein L is selected from the group consisting of substituted or unsubstituted structural fragments composed of one or more of the following groups: C₁₋₆ alkylene, 6- to 10-membered aryl, 5- to 6-membered heteroaryl, substituted or unsubstituted 9- to 12-membered nitrogen-containing heterocyclyl (e.g., substituted with one or more R′), —N(R′)—, —NH(R′), —N(R′)₂, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Lys(R′), Glu(R′), short peptides composed of amino acids (e.g., Gly-Lys, Asp-Gly-Gly-Phe-Gly, Glu-Gly-Gly-Phe-Gly, Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly), 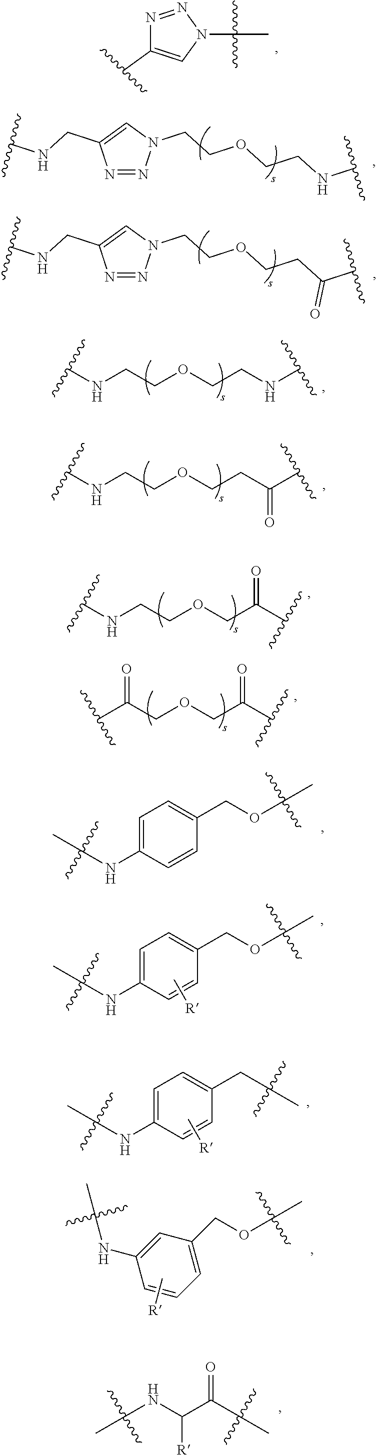 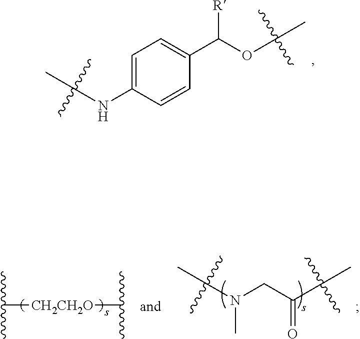
wherein R′ is composed of one or more of the following groups, including but not limited to hydrogen, C₁₋₆ alkyl, C₁₋₆ alkylene, amino, hydroxyl, carboxyl, acyl, —O—, —C₁₋₆ alkylene-CO₂H, —C₁₋₆ alkylene-SO₃H, —SO₃H, —PO₃H₂, —C₁₋₆ alkylene-NHC₁₋₆ alkyl, —C₁₋₆ alkylene-N(C₁₋₆ alkyl)₂, —CH₂N(C₁₋₆ alkyl)-C(═O)C₁₋₆ alkylene-heterocycle, —C₁₋₆ alkylene-heterocycle, —NHC₁₋₆ alkylene-SO₃H, 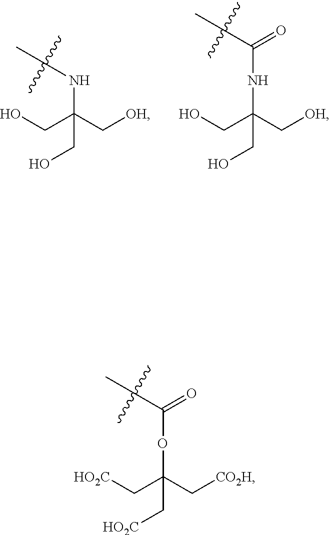 [SMILES: *NC(CO)(CO)CO ; *C(=O)NC(CO)(CO)CO ; *C(=O)OC(CC(=O)O)(CC(=O)O)CC(=O)O]
—CH₂NH—SO₃H, —CH₂N(C₁₋₆ alkyl)-SO₃H, —CH₂NHC₁₋₆ alkylene-SO₃H, —CH₂N(C₁₋₆ alkyl)C₁₋₆ alkylene-SO₃H, —CH₂N(C₁₋₆ alkylene-SO₃H)₂, —CH₂N⁺(C₁₋₆ alkylene-SO₃H)₃, —CH₂N⁺(C₁₋₆ alkyl)₂-C₁₋₆ alkylene-SO₃H, —CH₂N(C₁₋₆ alkyl)-C(═O)C₁₋₆ alkylene-N⁺(C₁₋₆ alkylene-SO₃H)₃, —CH₂NH—C(═O)C₁₋₆ alkylene-N⁺(C₁₋₆ alkylene-SO₃H)₃, —CH₂N(C₁₋₆ alkyl)-C(═O)C₁₋₆ alkylene-N⁺(C₁₋₆ alkyl)₃, —CH₂NH—C(═O)C₁₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₂-CH₂CO₂H, —CH₂N(C₁₋₆ alkyl)-C₁₋₆ alkylene-CO₂H, —CH₂N⁺(C₁₋₆ alkyl)₂-C₁₋₆ alkylene-CO₂H, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—(OCH₂CH₂)ᵣ—OC₁₋₆ alkyl, —(CH₂N(Me)-C(═O))ᵣ—C₁₋₆ alkyl, polyethylene glycol fragment containing 1 to 10 EO units (i.e., —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), NOTA (1,4,7-triazacyclononane-N,N′N″-triacetic acid residue), —C₁₋₆ alkylene-N(C₁₋₆ alkyl)-DOTA, —C₁₋₆ alkyl-N(C₁₋₆ alkyl)-DOTAGA, or —C₁₋₆ alkyl-N(C₁₋₆ alkyl)-NOTA, wherein r is an integer selected from 1 to 20, such as an integer of 1 to 15, 1 to 12, 3 to 12, 1 to 10, 1 to 8, 3 to 8, 1 to 6, 1 to 4, 1 to 2, such as 1, 2, 3, 4, 5, 6, 7, 8, 9, 10, 11, 12, 13, 14, 15, 16, 17, 18, 19 or 20; s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20;
preferably, L is selected from the group consisting of substituted or unsubstituted structural fragments composed of one or more of the following groups: 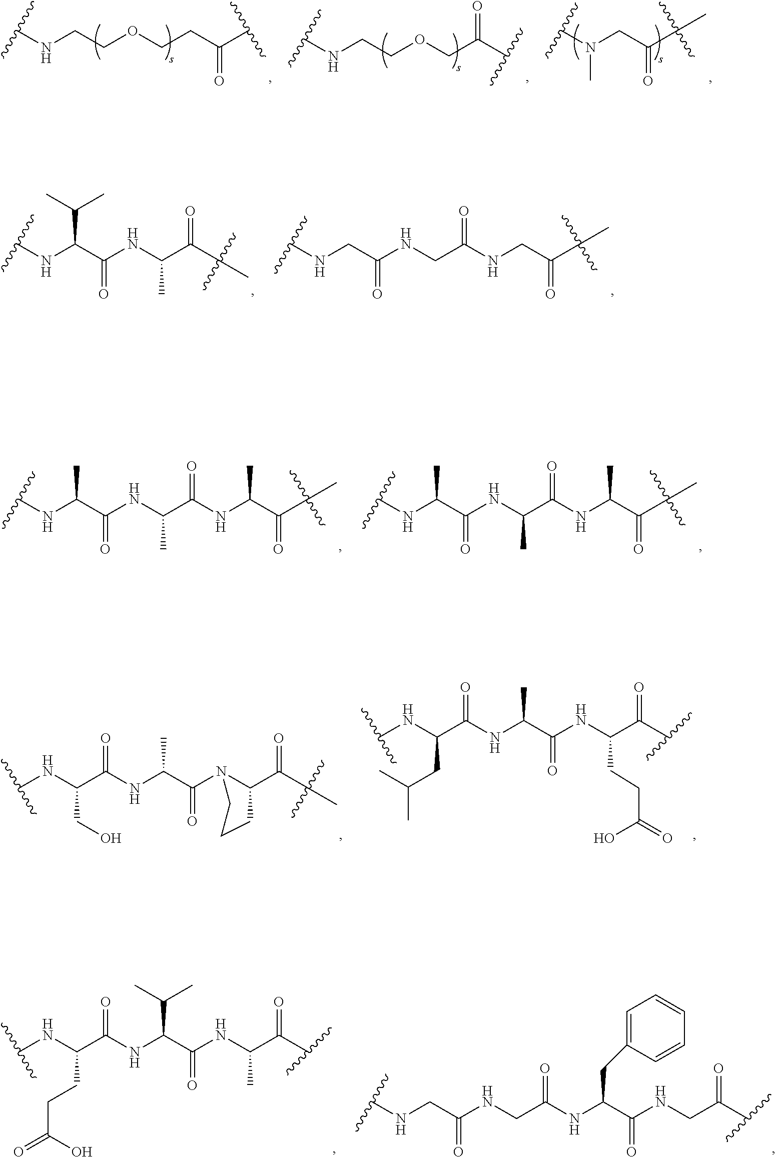 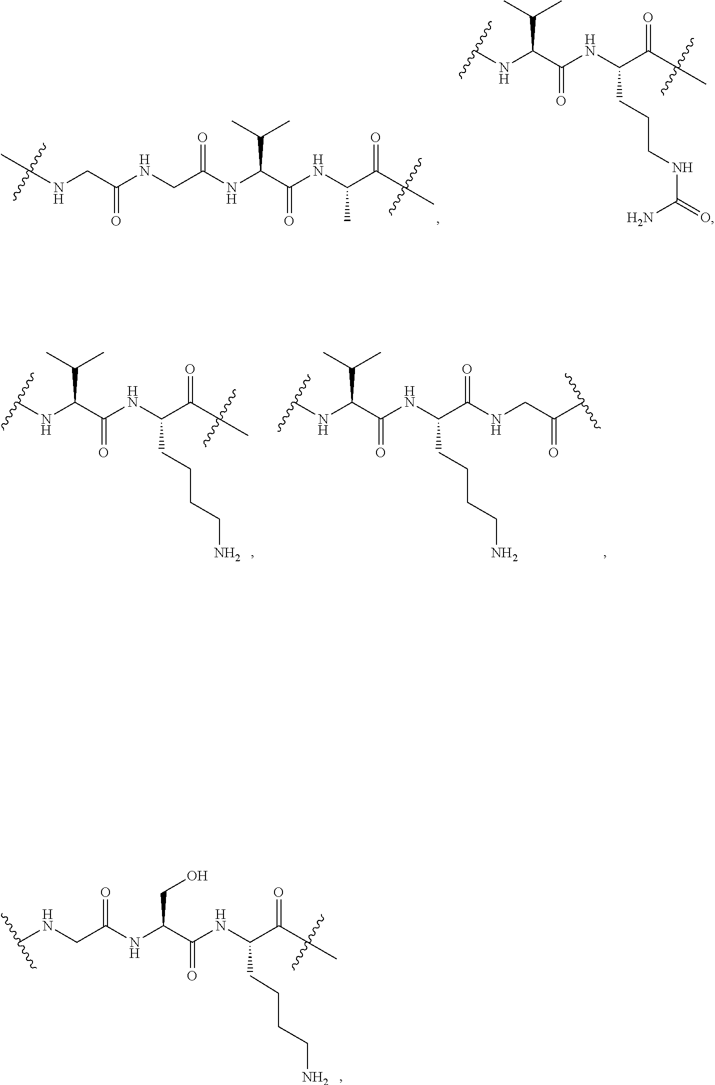 [SMILES: *NCC(=O)NCC(=O)N[C@H](C(=O)N[C@@H](C)C(*)=O)C(C)C ; *N[C@H](C(=O)N[C@@H](CCCNC(N)=O)C(*)=O)C(C)C ; *N[C@H](C(=O)N[C@@H](CCCCN)C(*)=O)C(C)C ; *N[C@H](C(=O)N[C@@H](CCCCN)C(=O)NCC(*)=O)C(C)C ; *NCC(=O)N[C@@H](CO)C(=O)N[C@@H](CCCCN)C(*)=O] 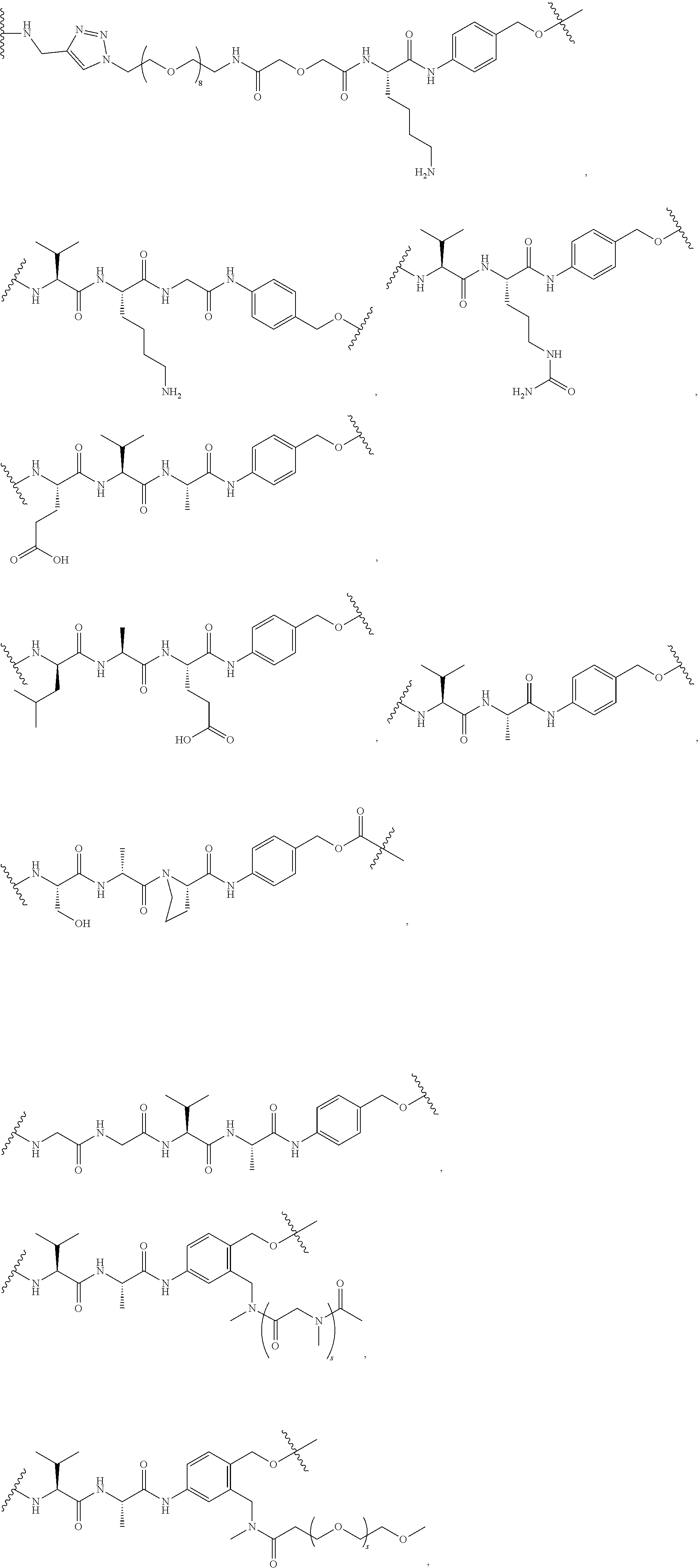 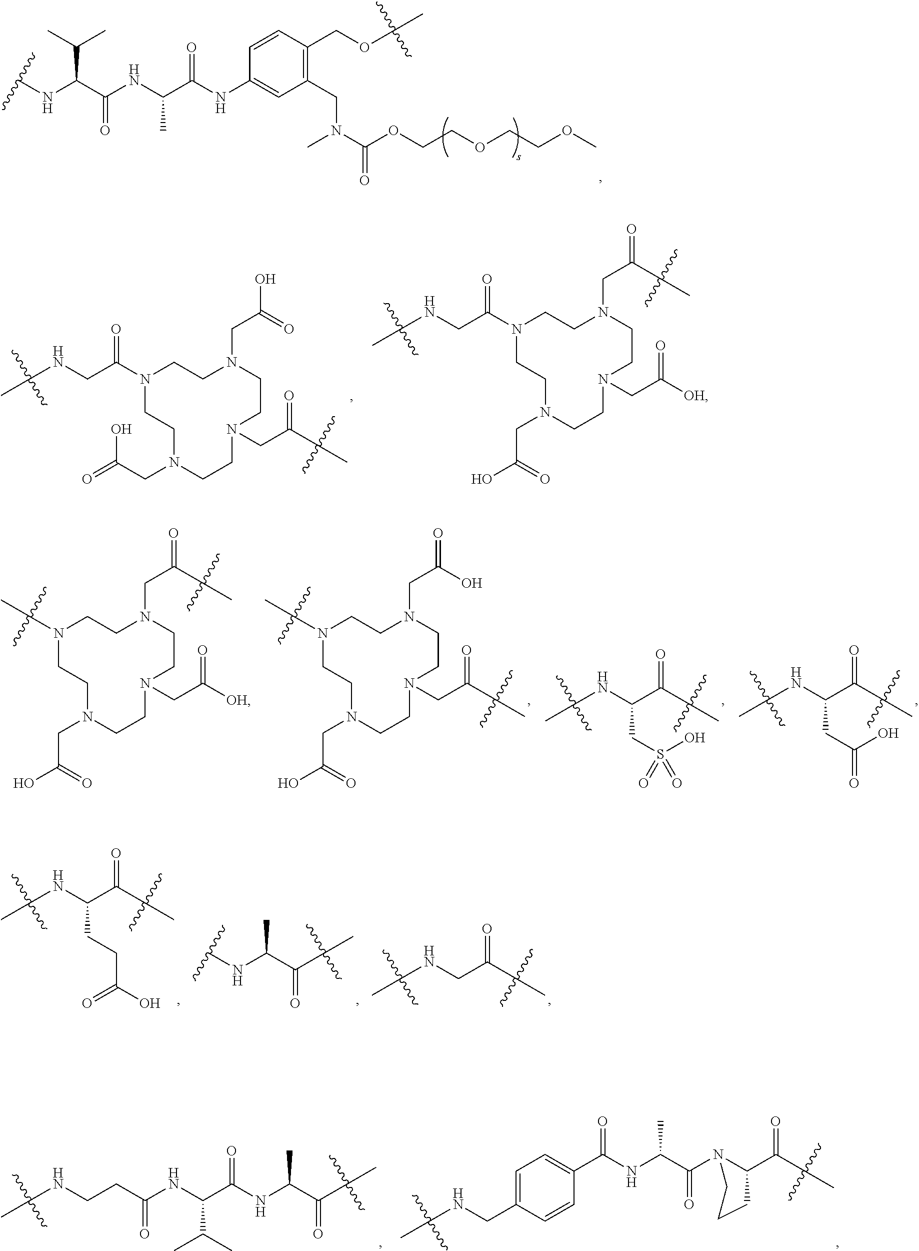 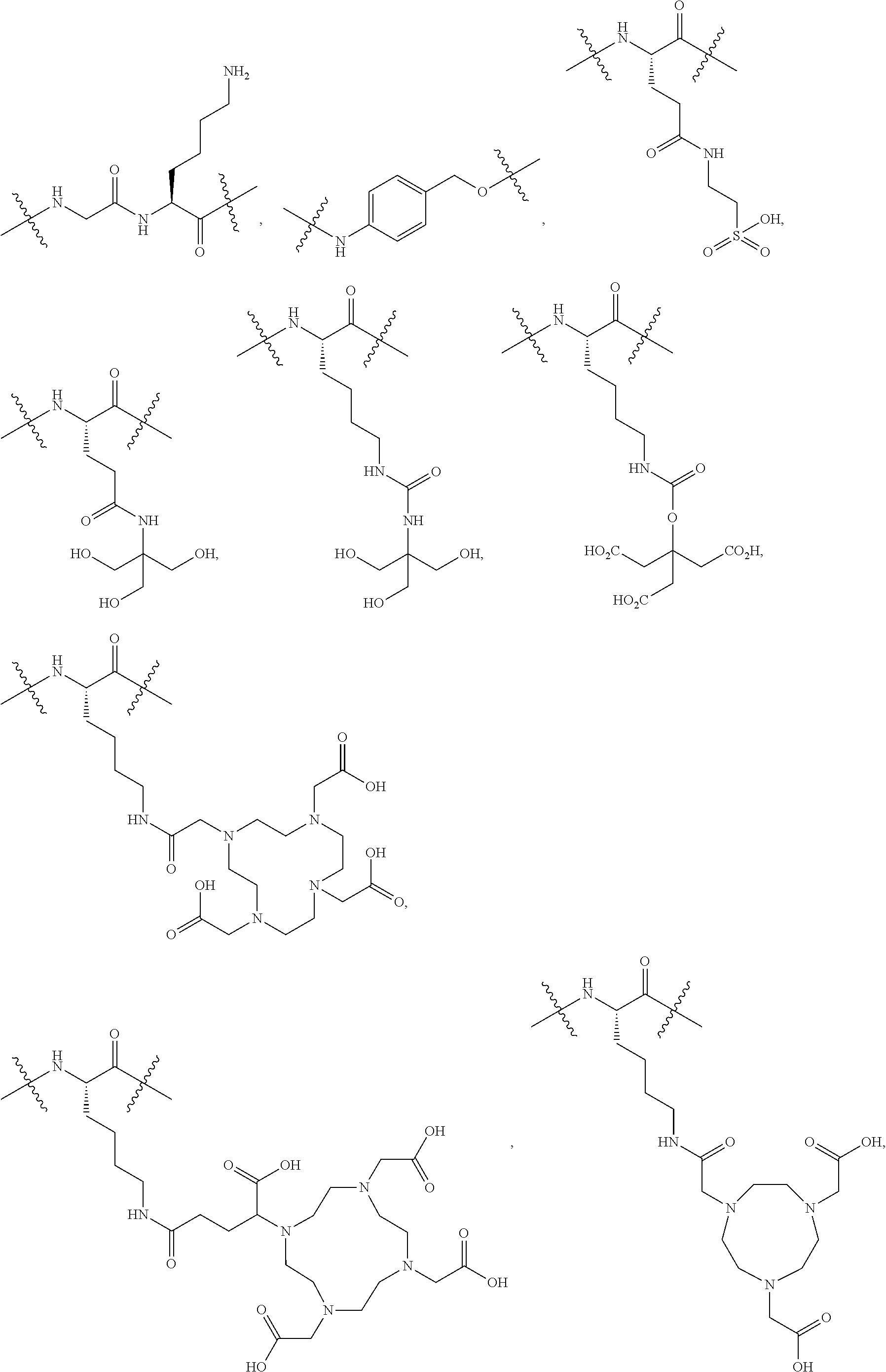 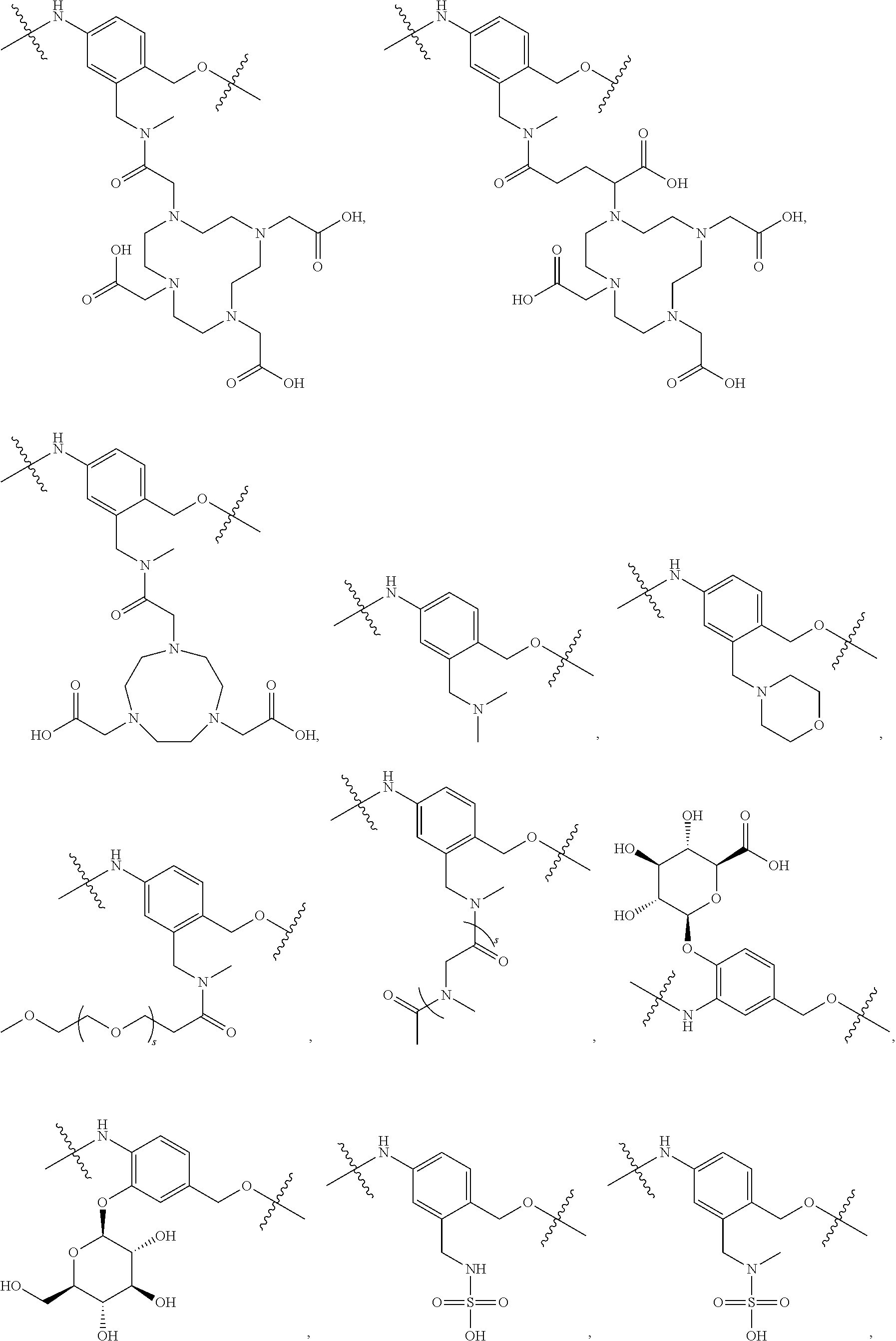 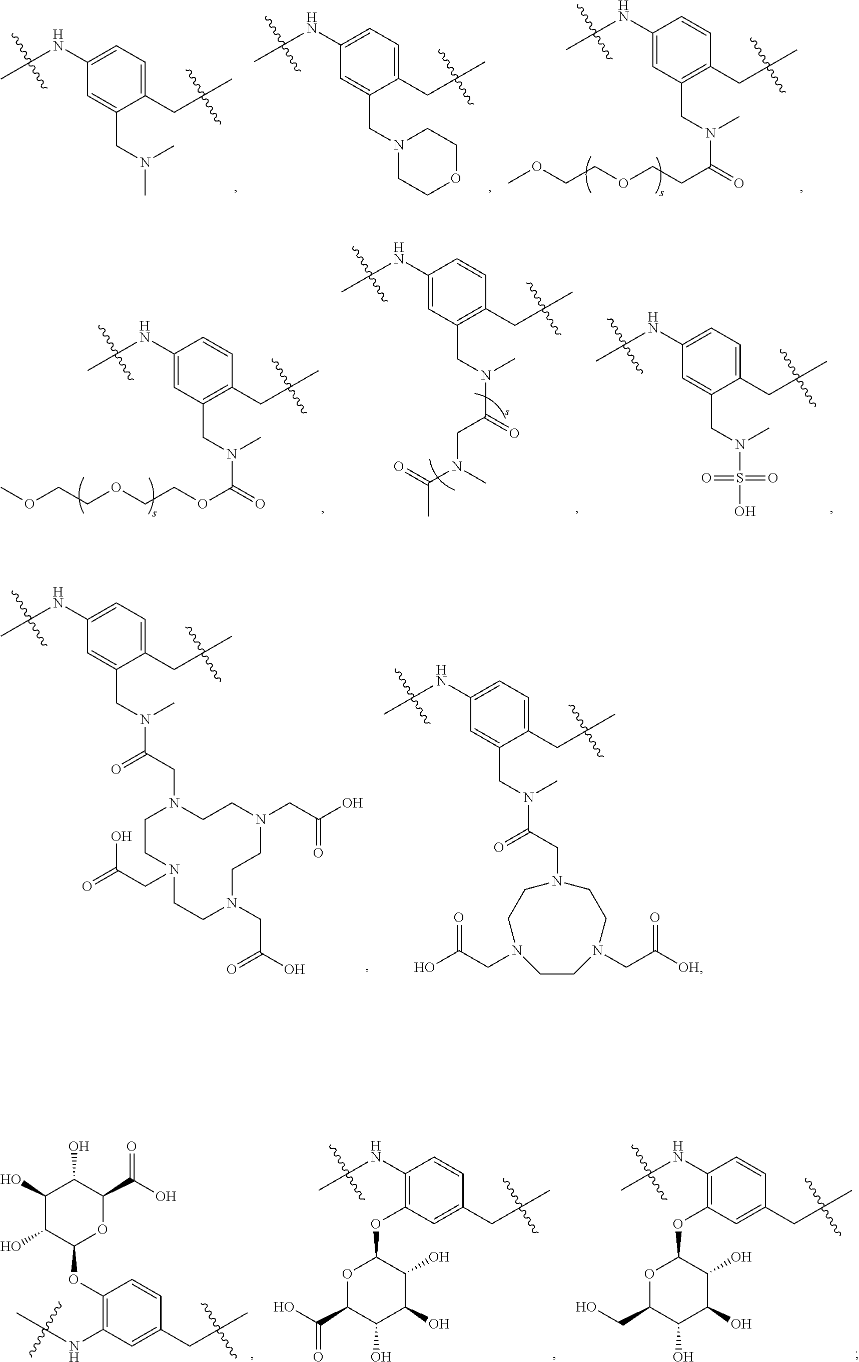
s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20;
preferably, L is selected from the group consisting of substituted or unsubstituted structural fragments composed of one or more of the following groups: 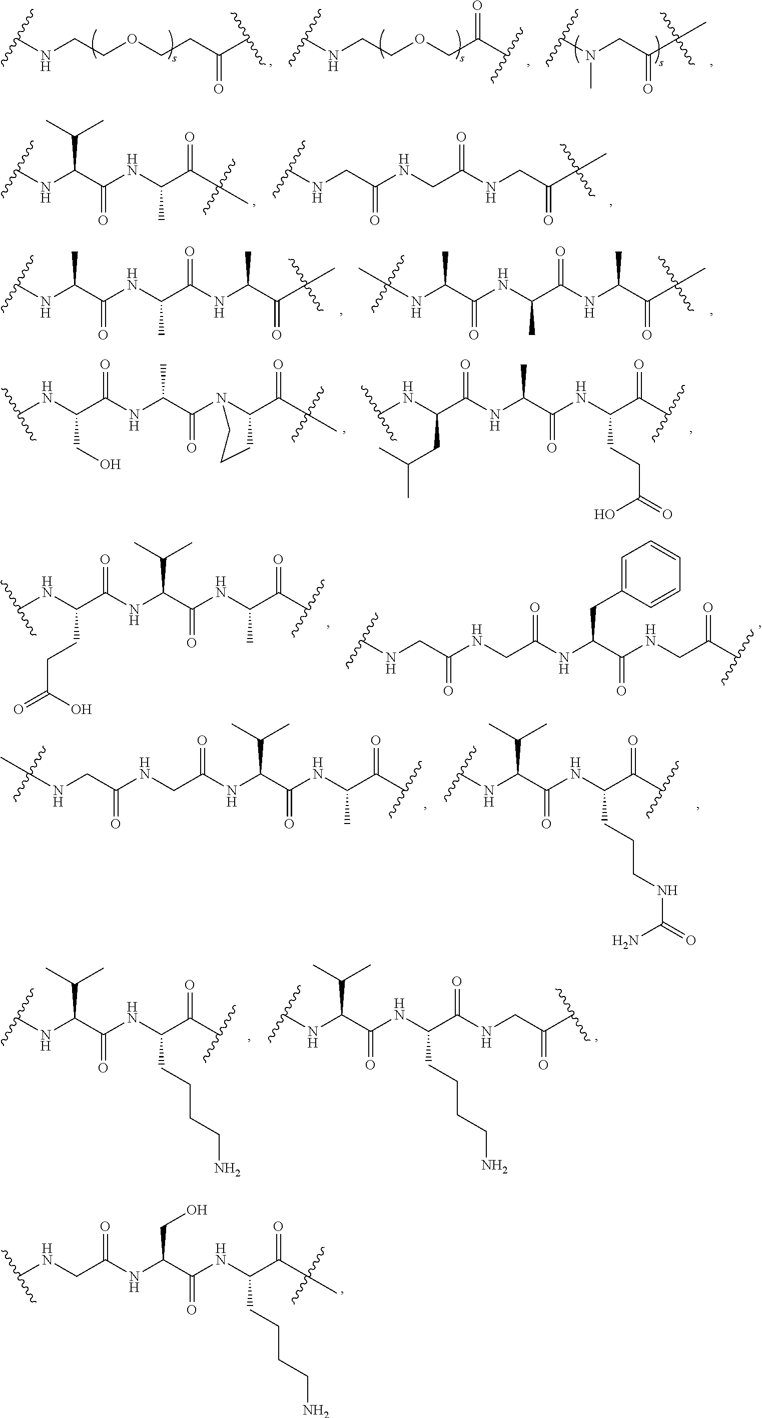 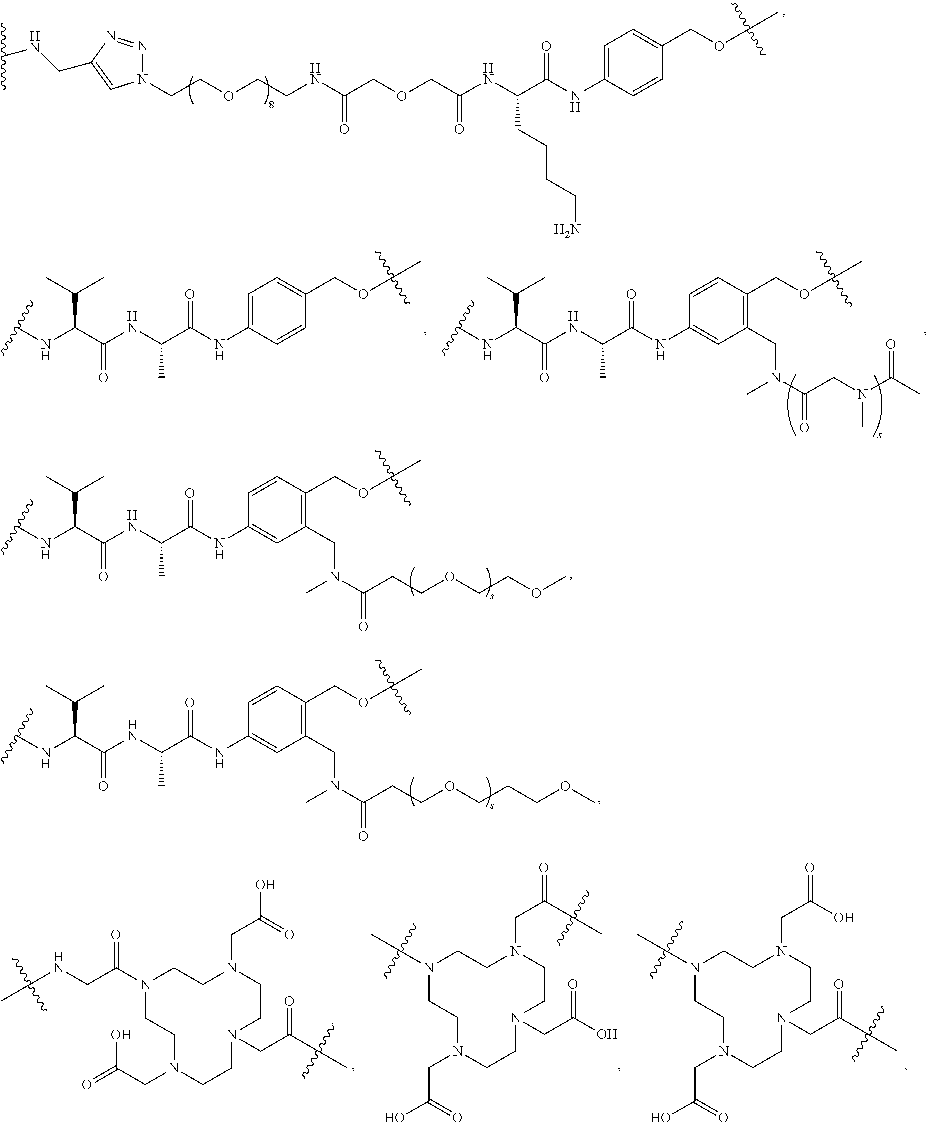 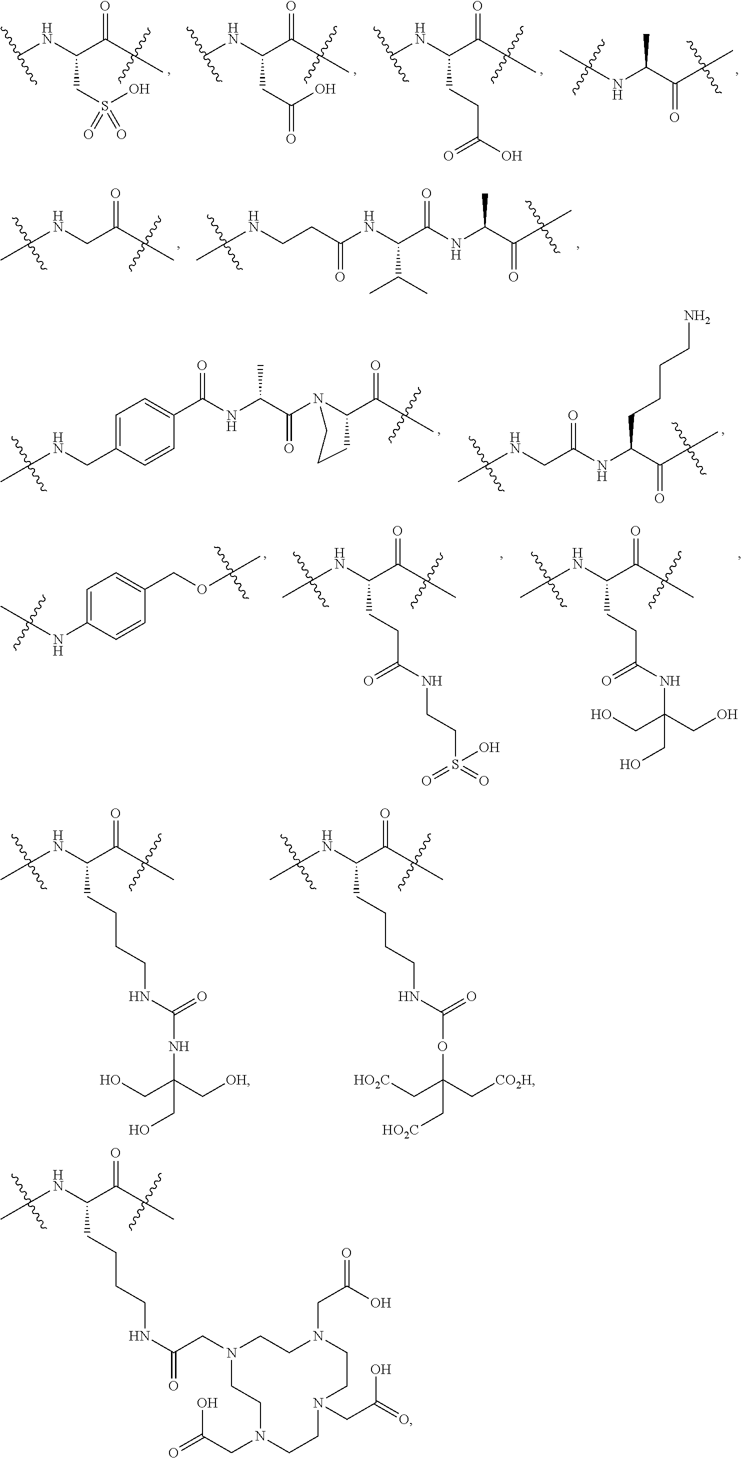 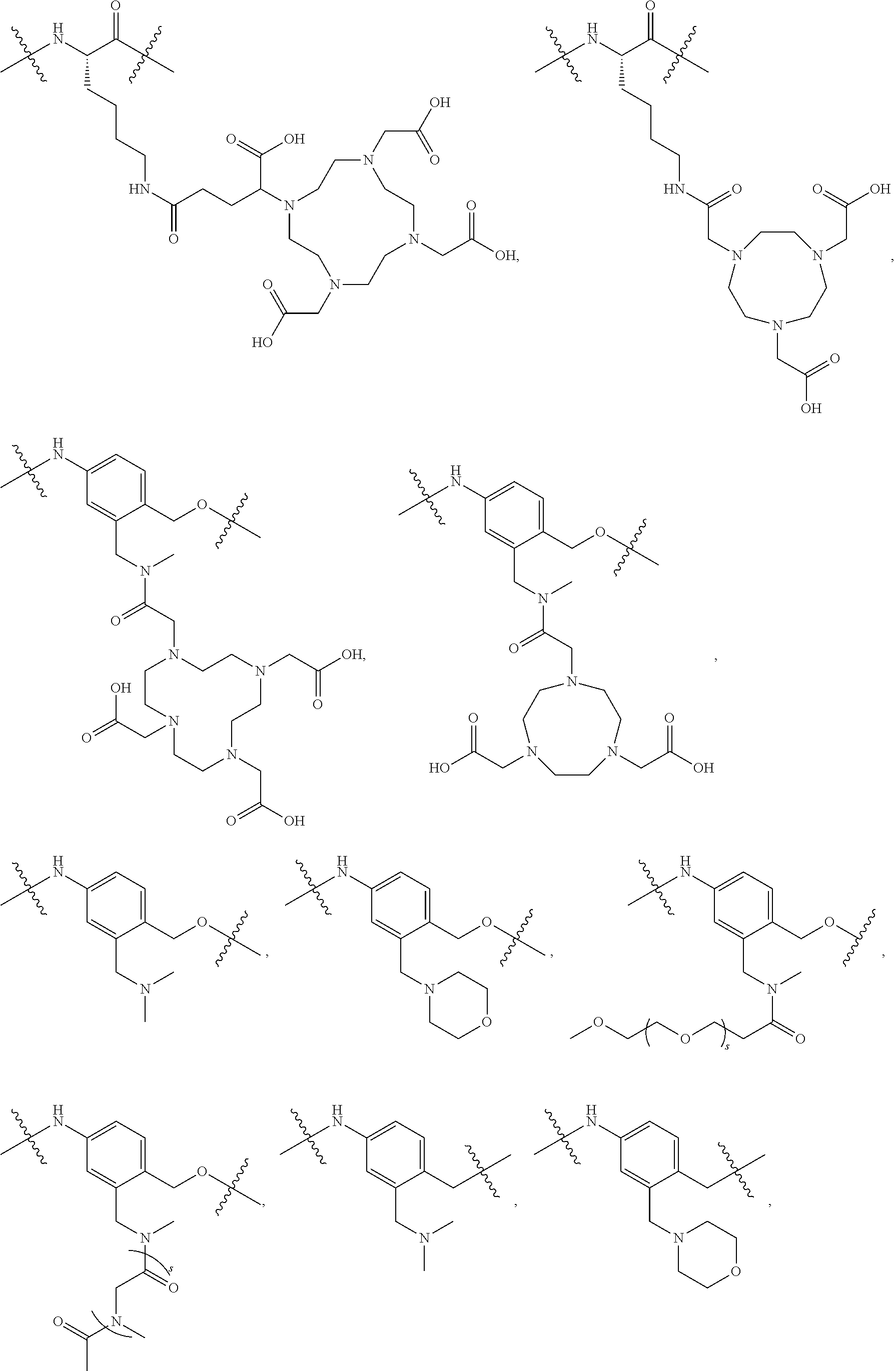 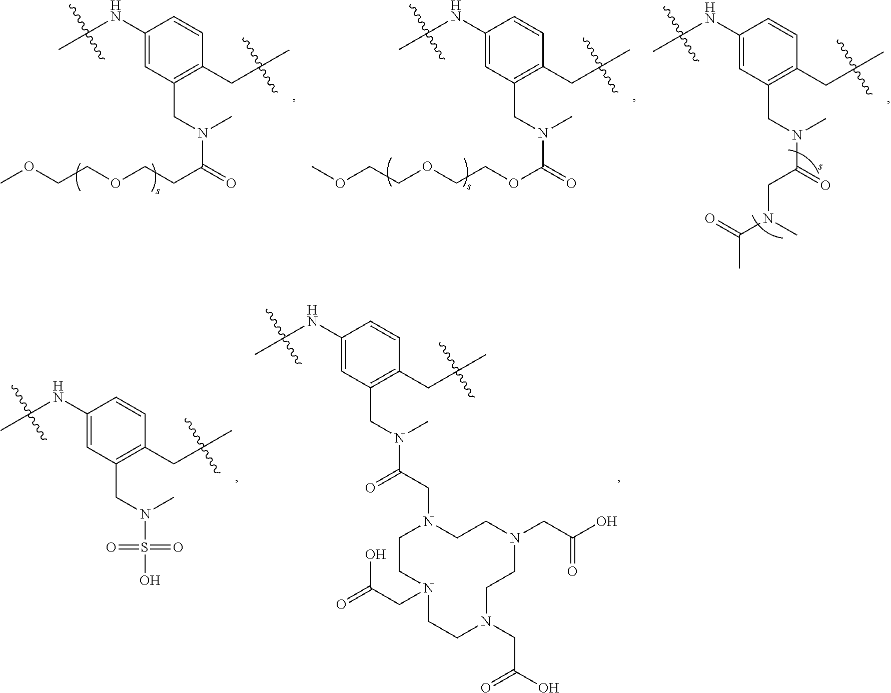 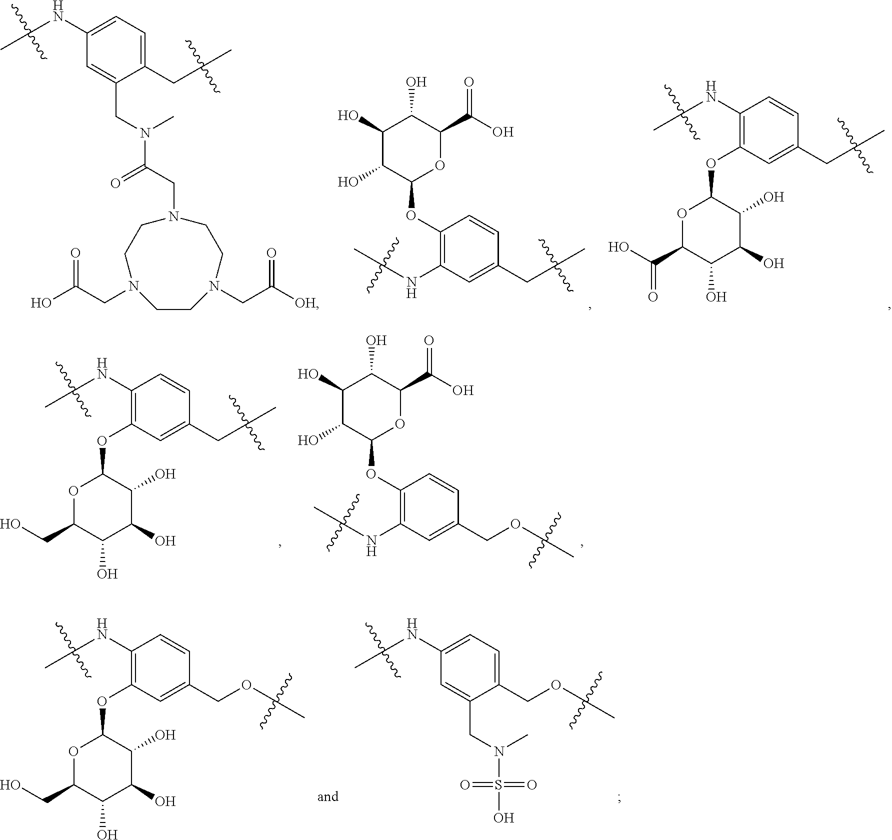
s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20;
preferably, L is selected from the group consisting of substituted or unsubstituted structural fragments composed of one or more of the following groups: 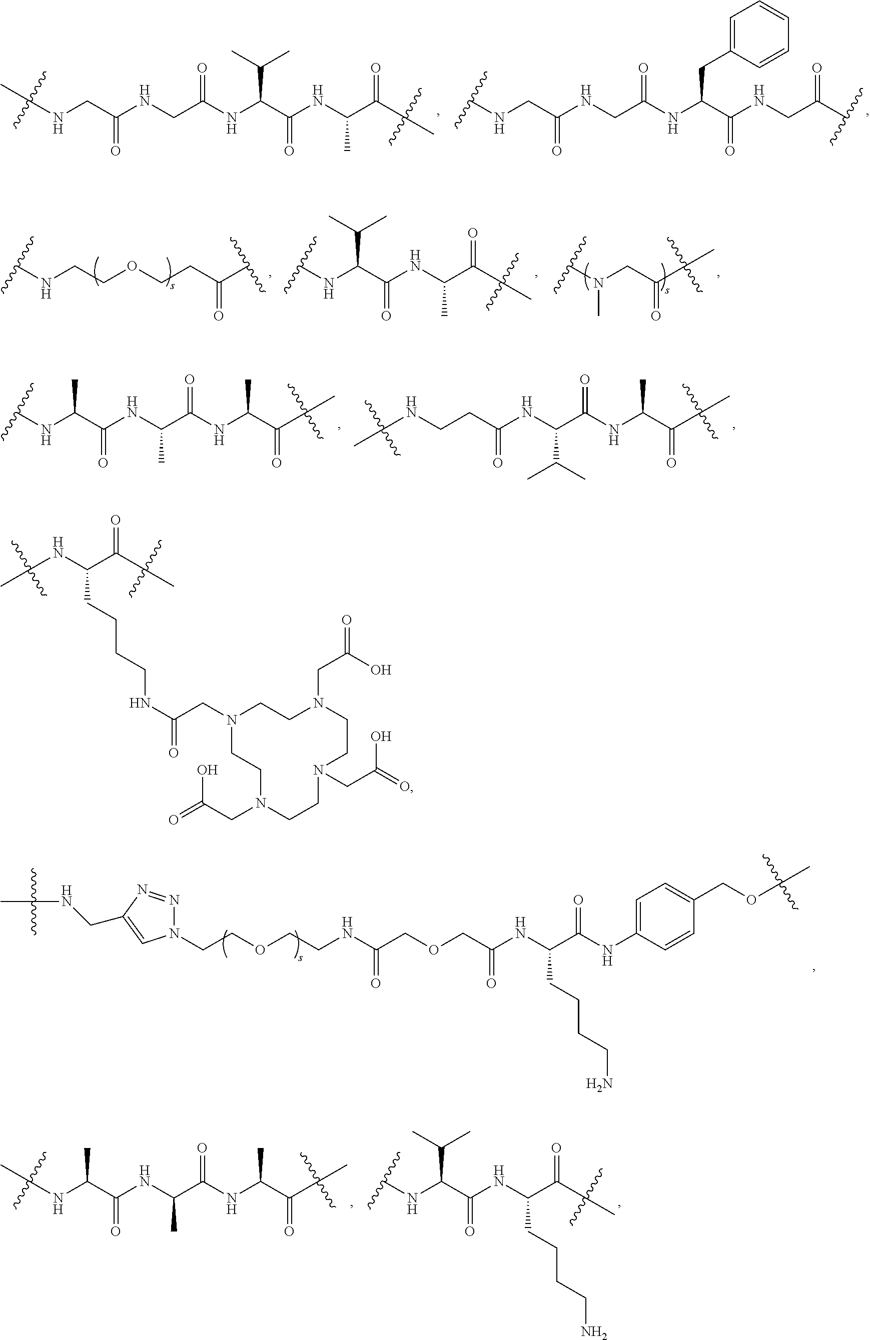 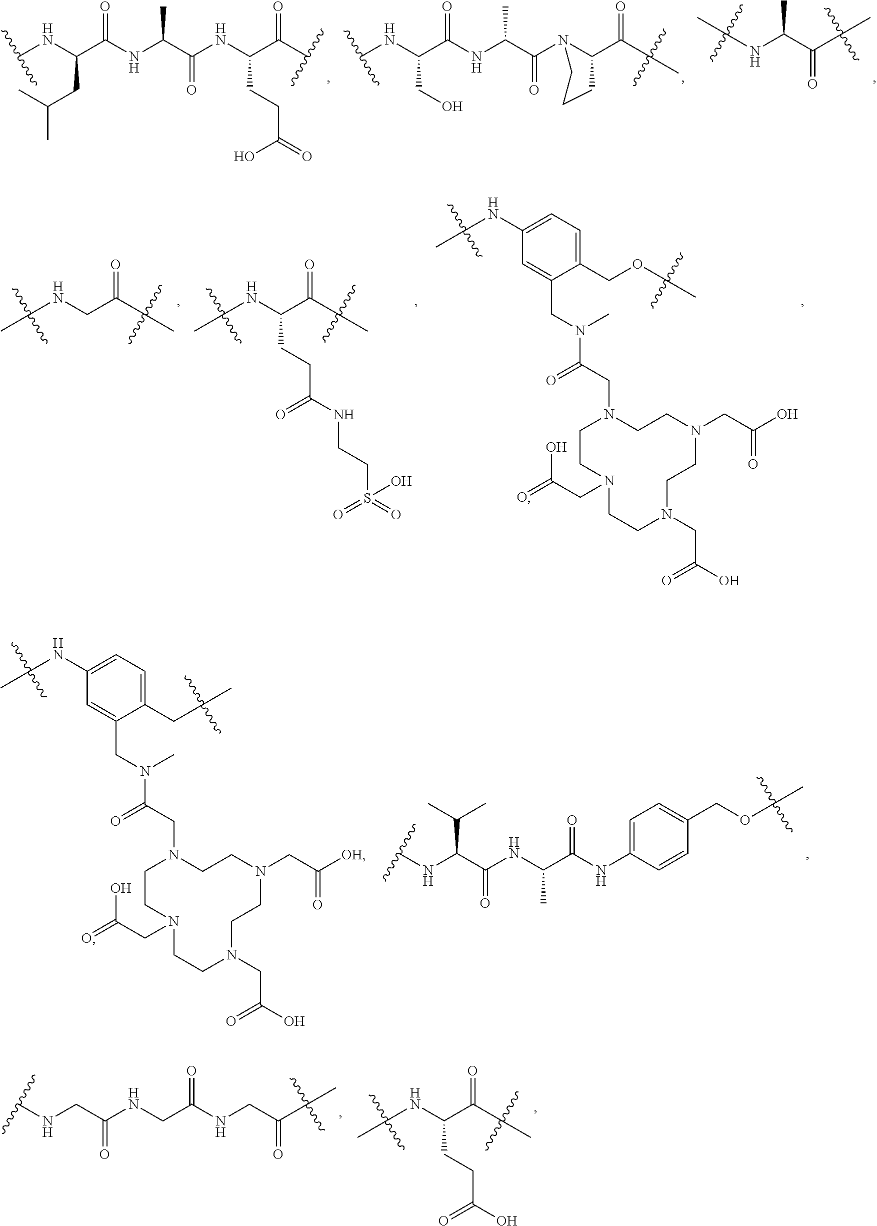 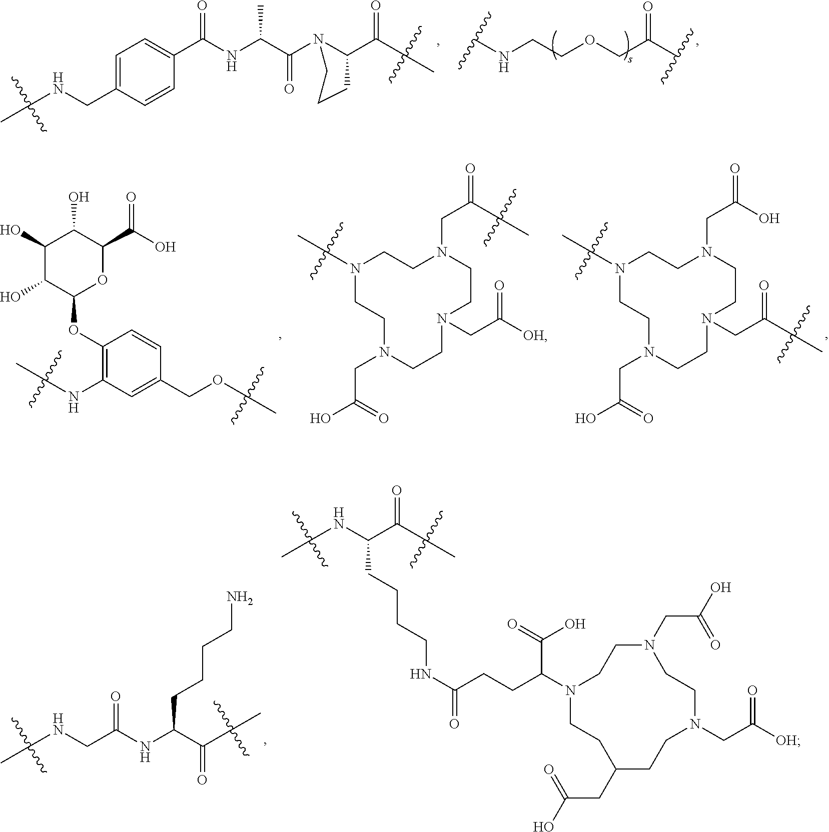
s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20;
preferably, L is selected from the group consisting of substituted or unsubstituted structural fragments composed of one or more of the following groups: 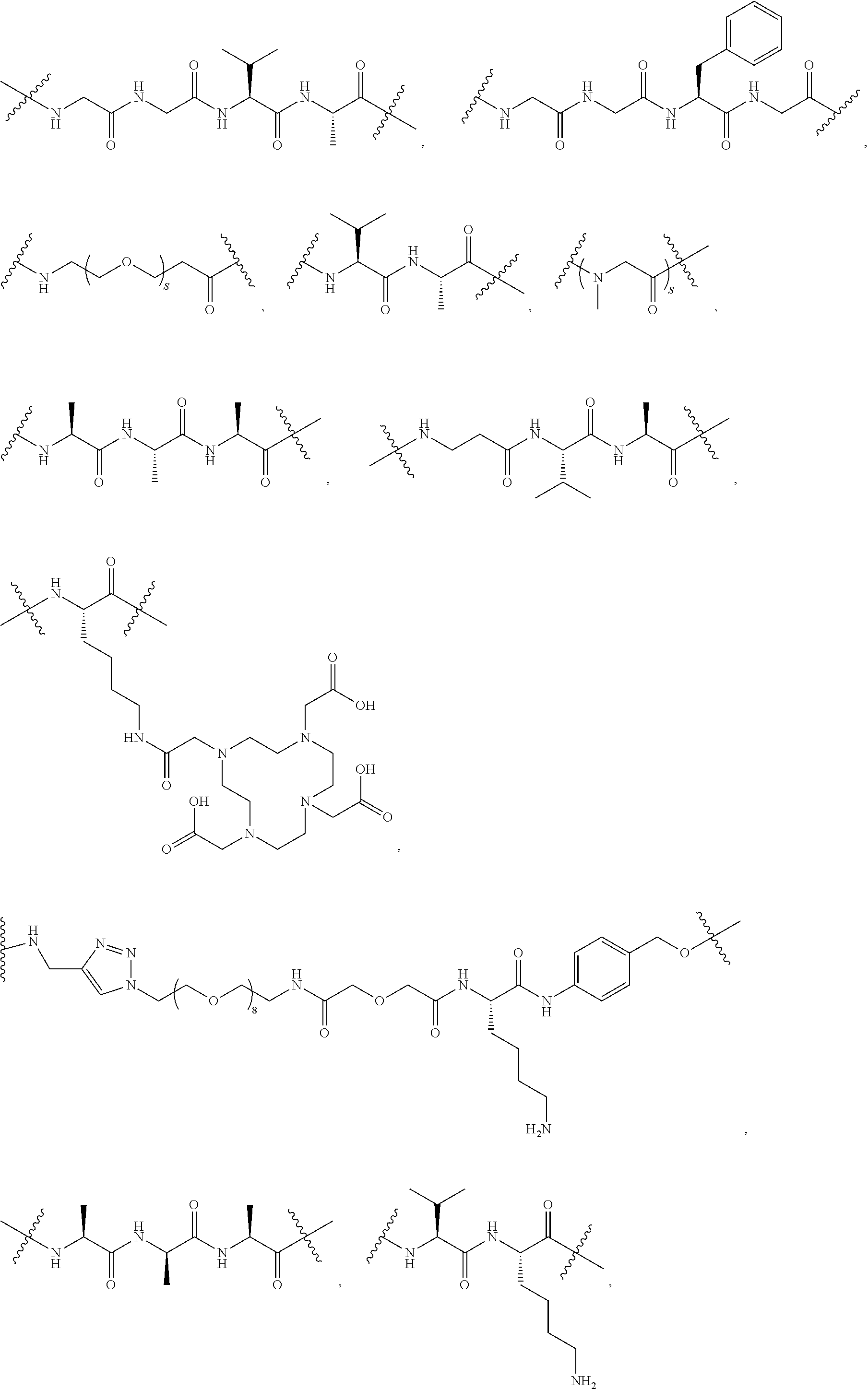 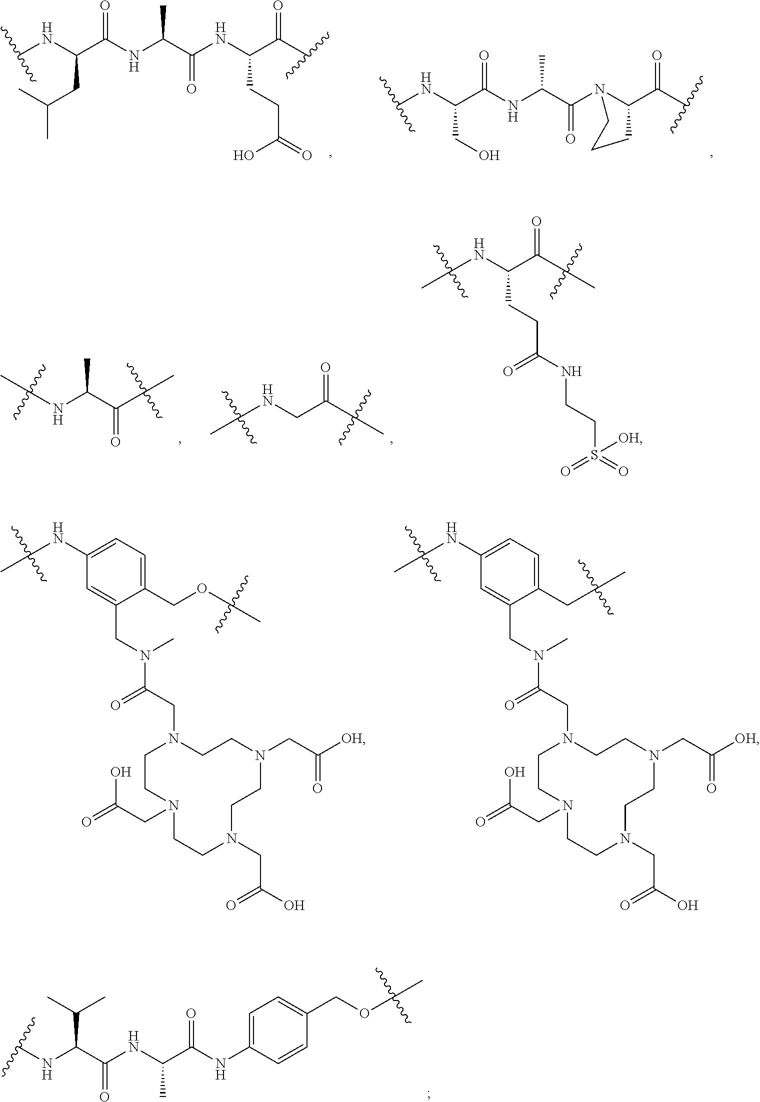
s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20;
preferably, L is selected from the group consisting of substituted or unsubstituted structural fragments composed of one or more of the following groups: 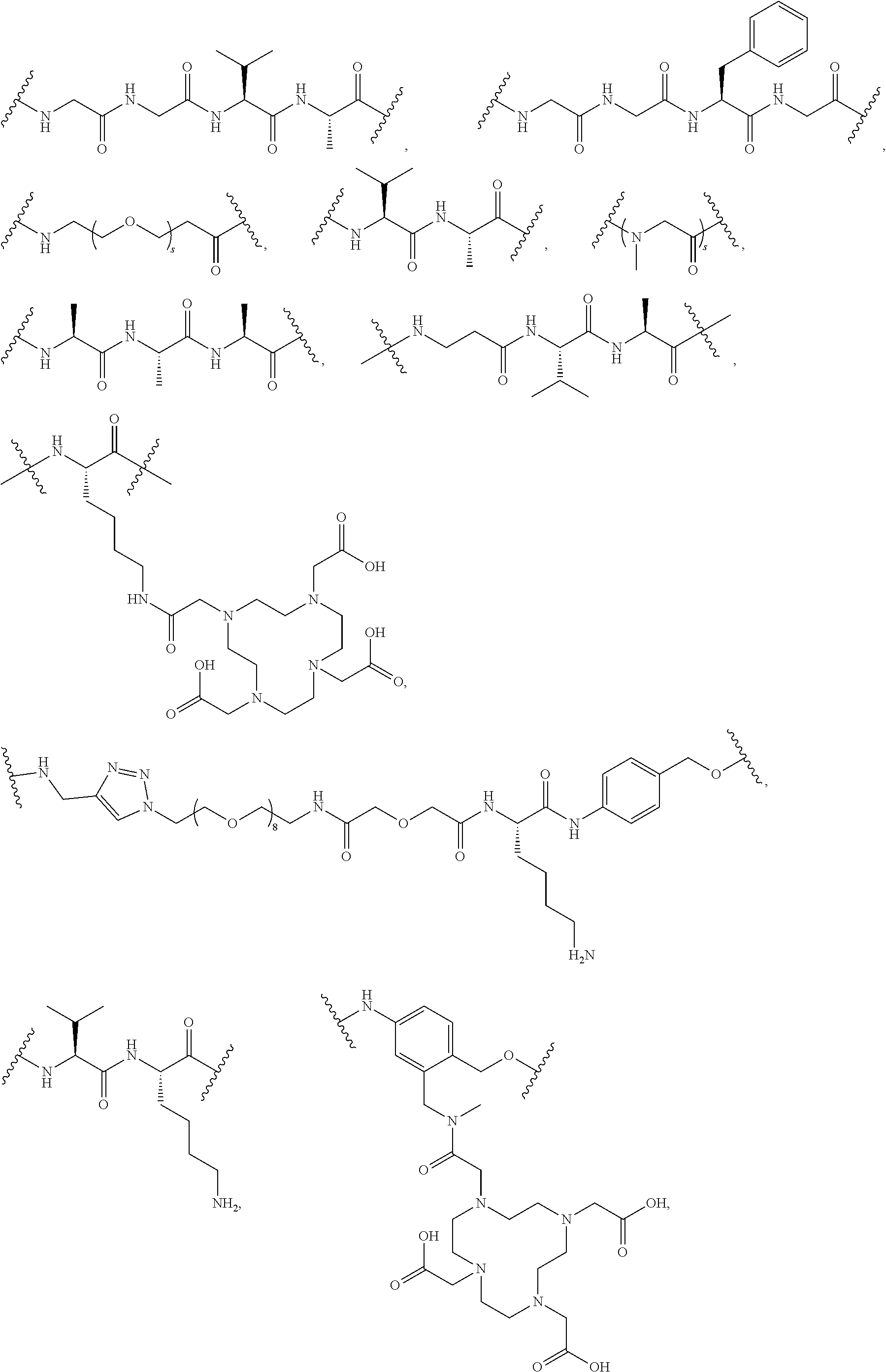 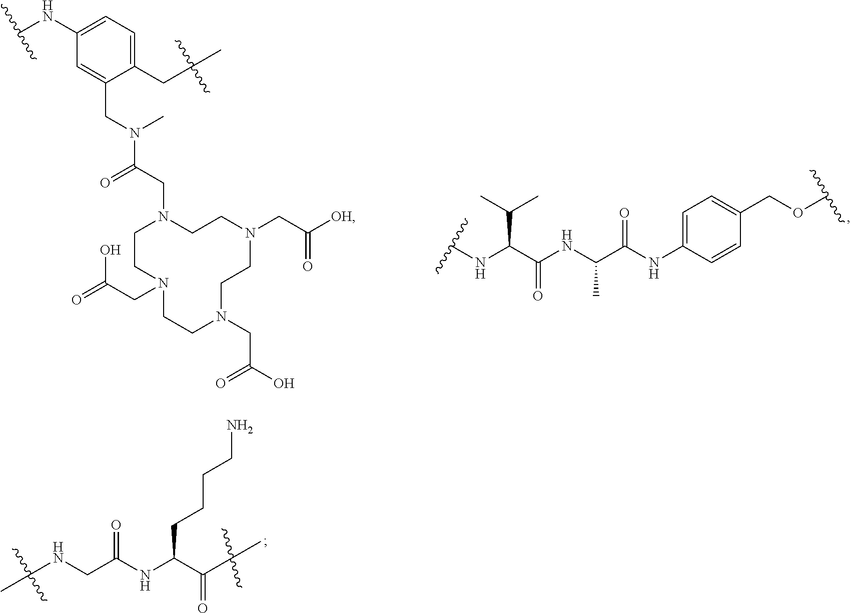
s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20;
L is selected from the group consisting of substituted or unsubstituted structural fragments composed of one or more (e.g., 1, 2, 3, 4, 5, 6 or 7) of the following groups: C₁₋₆ alkylene, —N(R′)—, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Lys(R′), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly), 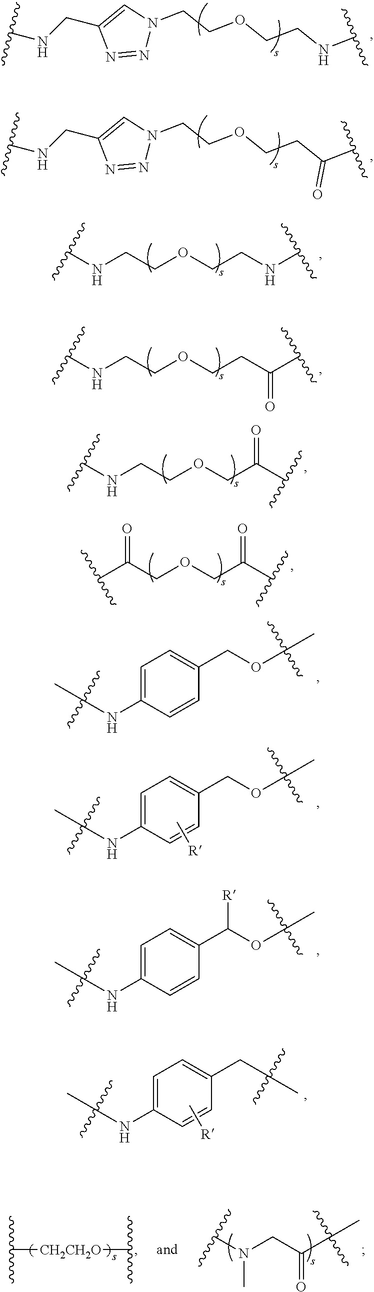
wherein R′ is composed of one or more (e.g., 1, 2, 3, 4, 5, 6, or 7) of the following groups, including but not limited to hydrogen, —C₁₋₆ alkylene-NHC₁₋₆ alkyl, —C₁₋₆ alkylene-N(C₁₋₆ alkyl)₂, DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), or NOTA (1,4,7-triazacyclononane-N,N′,N″-triacetic acid residue), wherein s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20;
preferably, L is selected from the group consisting of substituted or unsubstituted structural fragments composed of one or more (e.g., 1, 2, 3, 4 or 5) of the following groups: 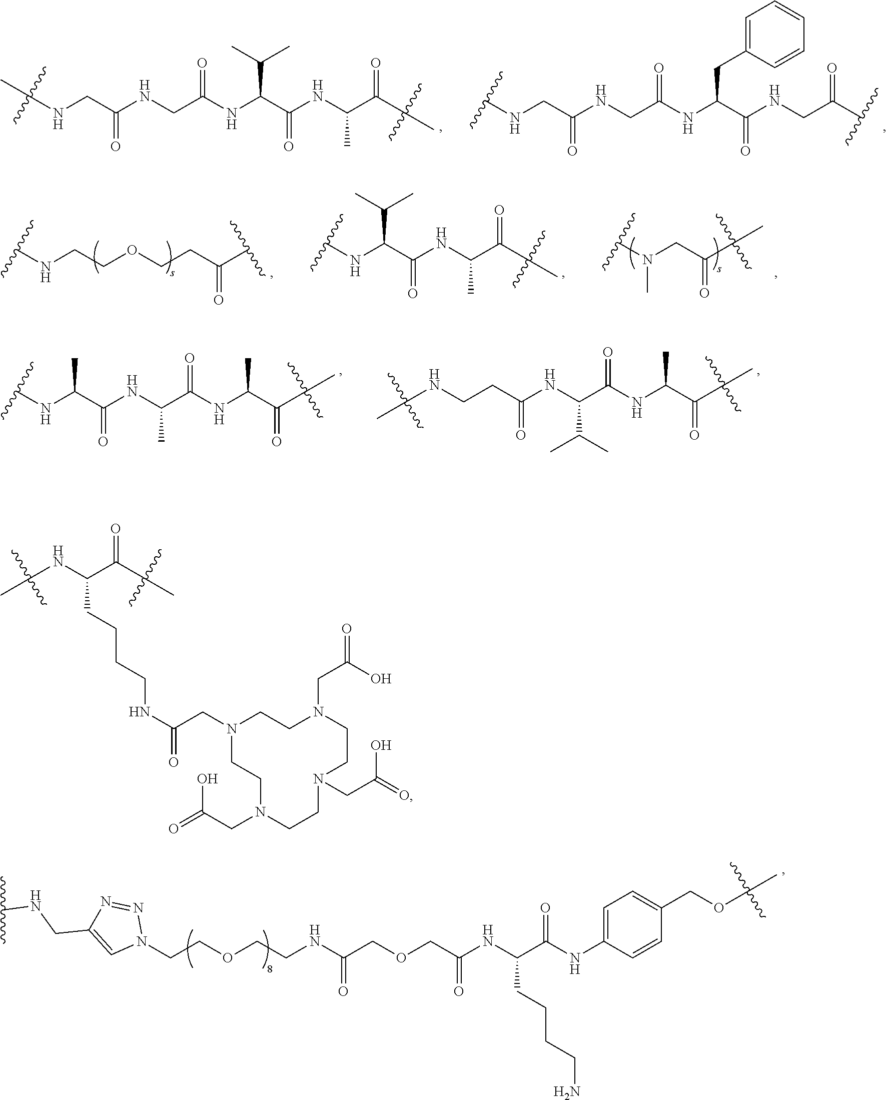 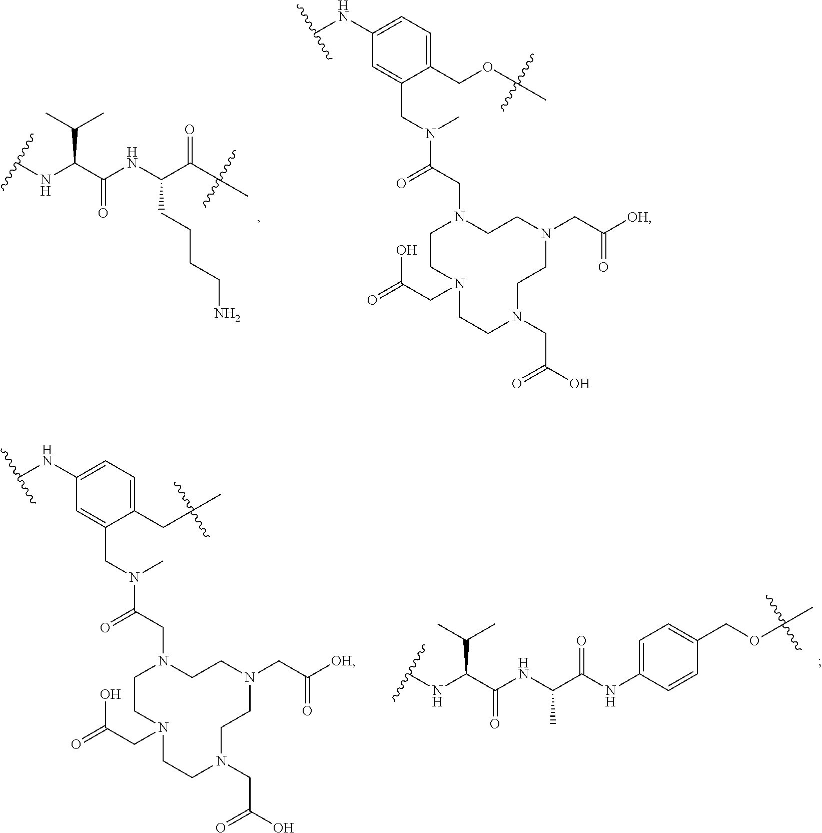 [SMILES: *N[C@H](C(=O)N[C@@H](CCCCN)C(*)=O)C(C)C ; *Nc1ccc(CO*)c(CN(C)C(=O)CN2CCN(CC(=O)O)CCN(CC(=O)O)CCN(CC(=O)O)CC2)c1 ; *Cc1ccc(N*)cc1CN(C)C(=O)CN1CCN(CC(=O)O)CCN(CC(=O)O)CCN(CC(=O)O)CC1 ; *N[C@H](C(=O)N[C@@H](C)C(=O)Nc1ccc(CO*)cc1)C(C)C]
s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20;
preferably, L is selected from the group consisting of substituted or unsubstituted structural fragments composed of one or more (e.g., 1, 2, 3, 4 or 5) of the following groups 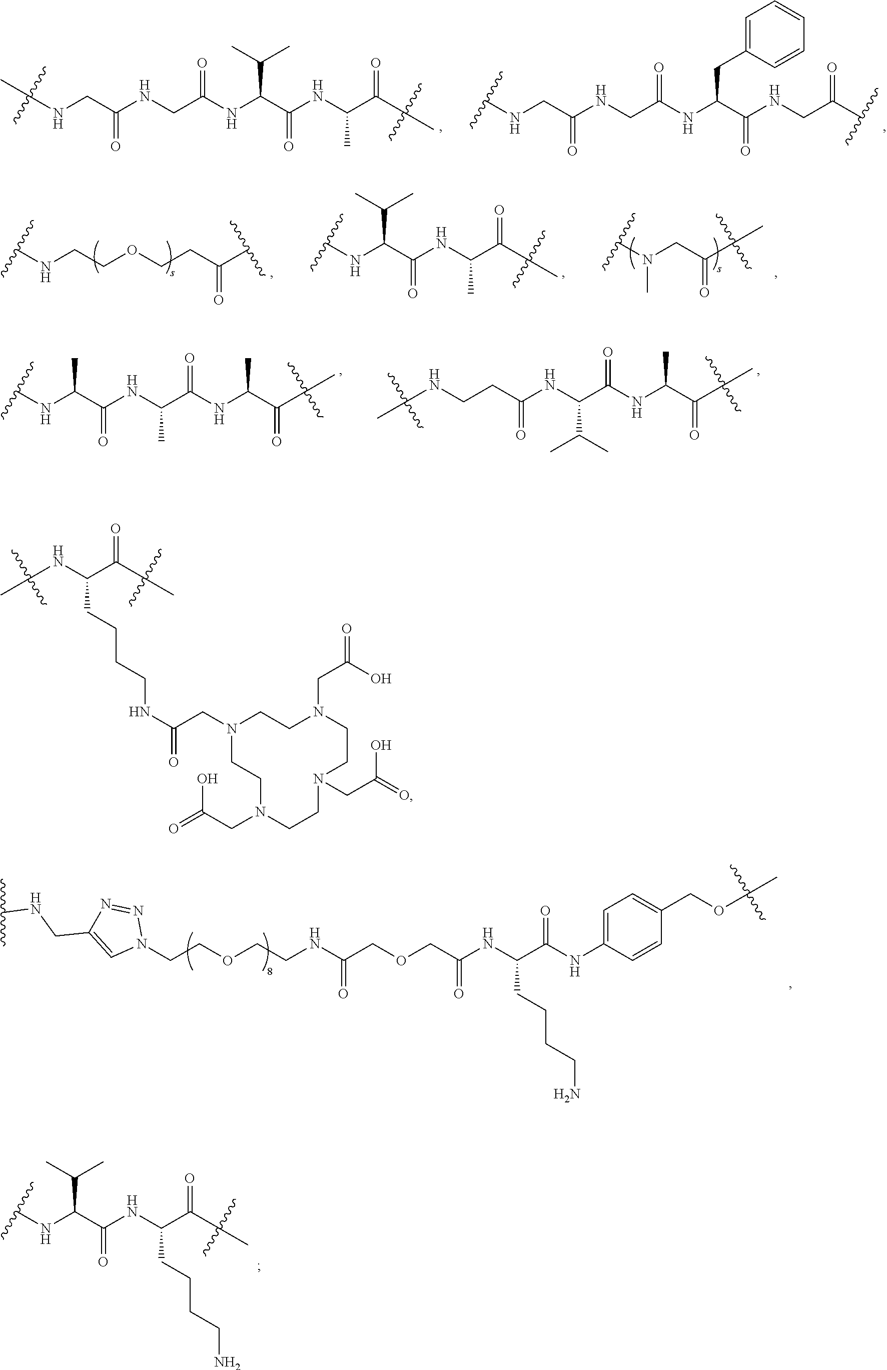
s is an integer selected from 1 to 20, such as an integer of 1 to 15, 1 to 12, 3 to 12, 5 to 10, 8 to 10, such as 1, 2, 3, 4, 5, 6, 7, 8, 9, 10, 11, 12, 13, 14, 15, 16, 17, 18, 19 or 20, preferably 8, 10.

5. The compound or pharmaceutically acceptable salt thereof according to claim 1, wherein E is a single bond, substituted or unsubstituted —NH—CH₂—, or is selected from the group consisting of the following substituted or unsubstituted structural fragments: 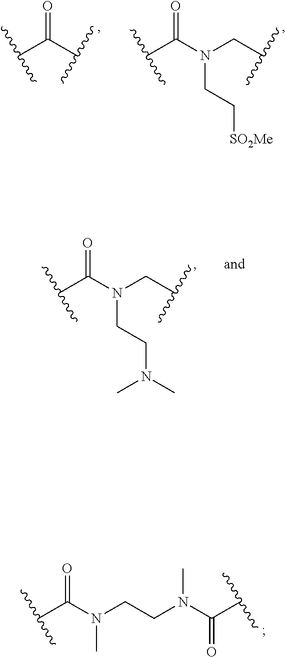
preferably, E is a single bond, substituted or unsubstituted —NH—CH₂—, 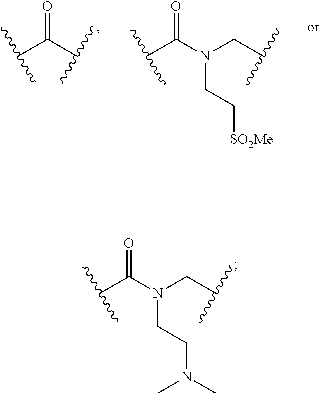
preferably, E is a single bond, substituted or unsubstituted —NH—CH₂—, or

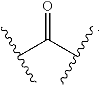 [SMILES: *C(*)=O]

6. The compound or pharmaceutically acceptable salt thereof according to claim 1, wherein the cytotoxic drug is selected from the group consisting of anti-tubulin agents, DNA intercalators, DNA topoisomerase inhibitors, RNA polymerase inhibitors and gene transcription inhibitors;
preferably, the cytotoxic drug is a topoisomerase I inhibitor (e.g., camptothecin, hydroxycamptothecin, 9-aminocamptothecin, SN-38, irinotecan, exatecan, topotecan, belotecan, rubitecan, diflomotecan, lurtotecan, karenitecin, gimatecan, namitecan, simmitecan, chimmitecan, silatecan, or elomotecan);
preferably, the cytotoxic drug is selected from the group consisting of the following compounds or isotopically labeled compounds thereof: 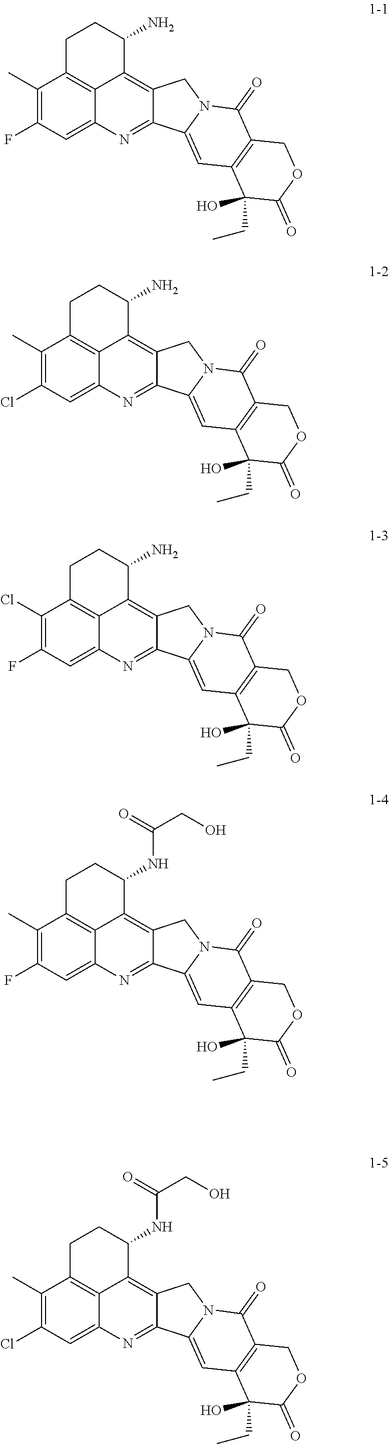 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3] 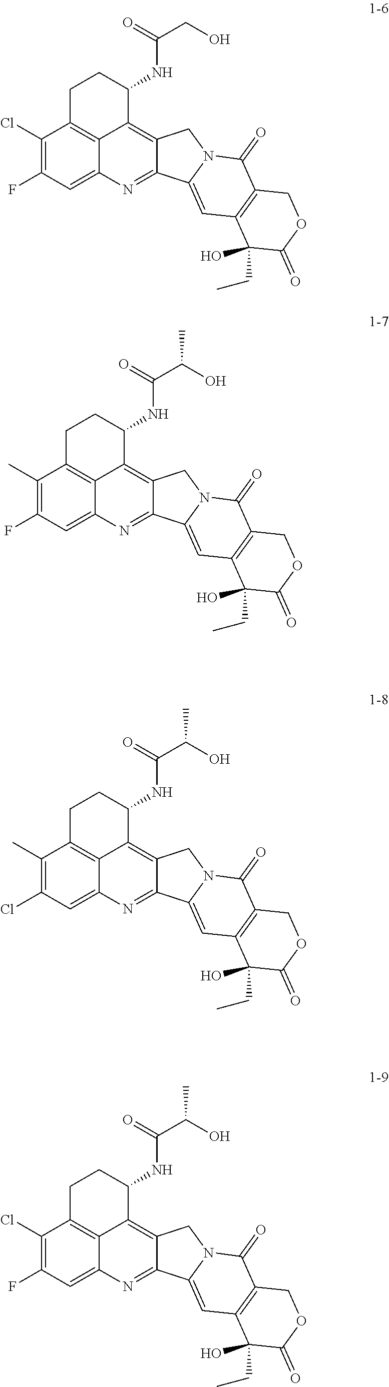 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3] 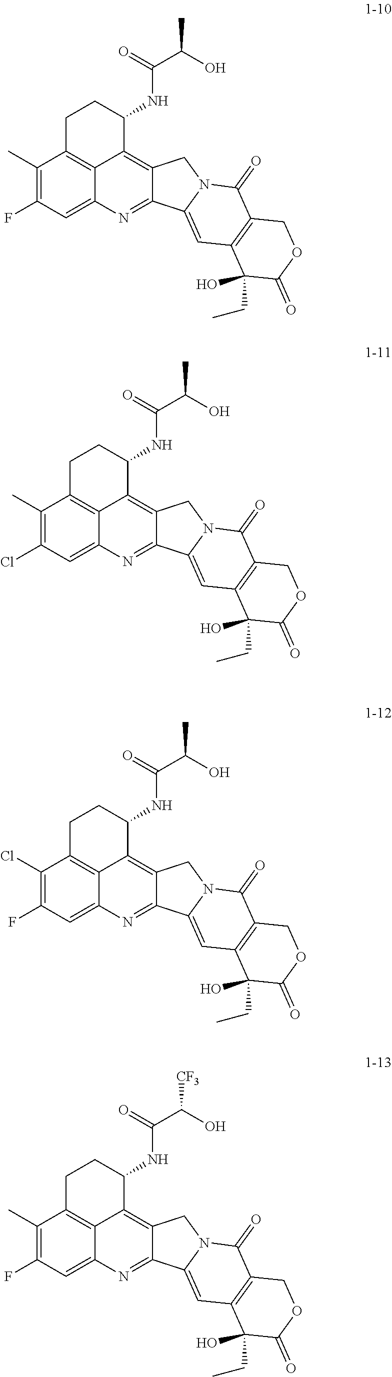 [SMILES: CC[C@@]1(O)C(=O)OCc2c1cc1n(c2=O)Cc2c-1nc1cc(F)c(C)c3c1c2[C@@H](NC(=O)[C@@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@@H](C)O)CC3 ; CC[C@@]1(O)C(=O)OCc2c1cc1n(c2=O)Cc2c-1nc1cc(F)c(C)c3c1c2[C@@H](NC(=O)[C@@H](O)C(F)(F)F)CC3] 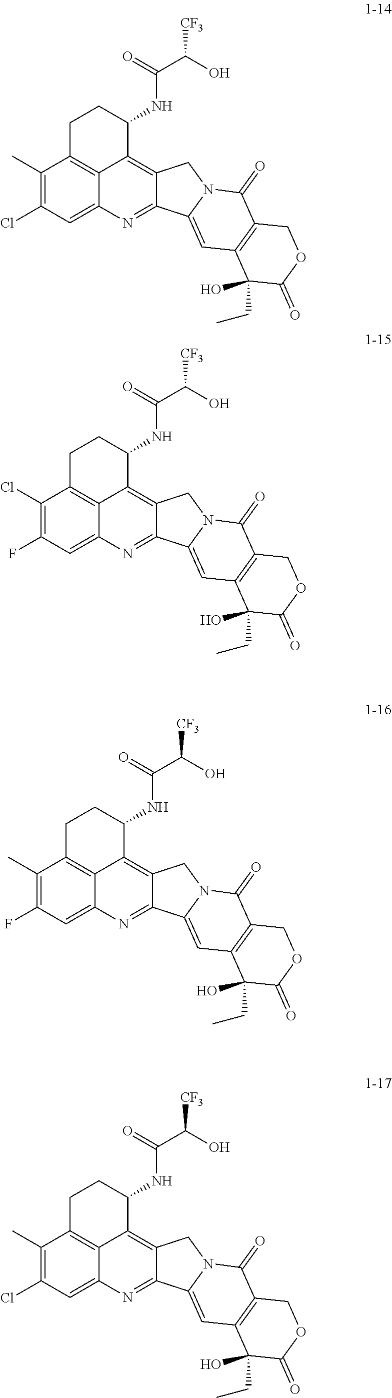 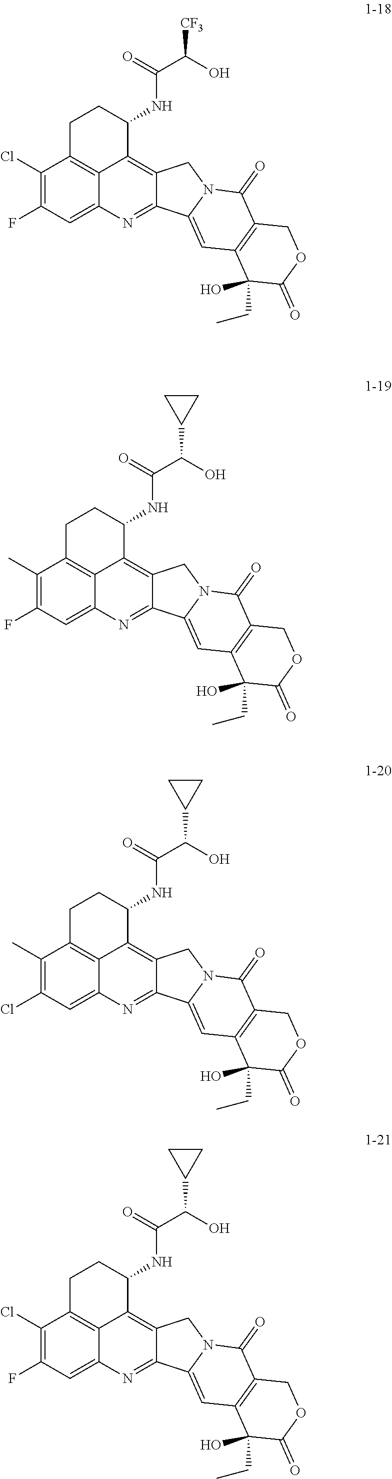 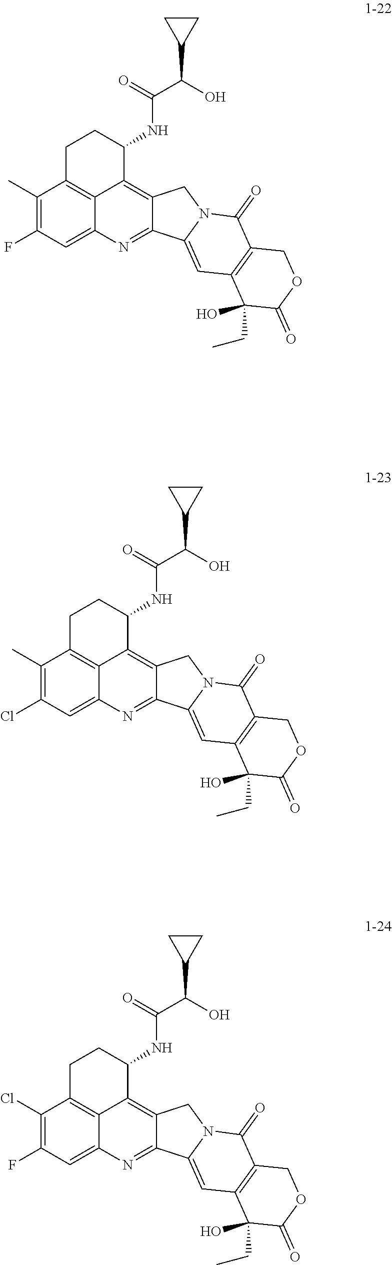 [SMILES: CC[C@@]1(O)C(=O)OCc2c1cc1n(c2=O)Cc2c-1nc1cc(F)c(C)c3c1c2[C@@H](NC(=O)[C@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3] 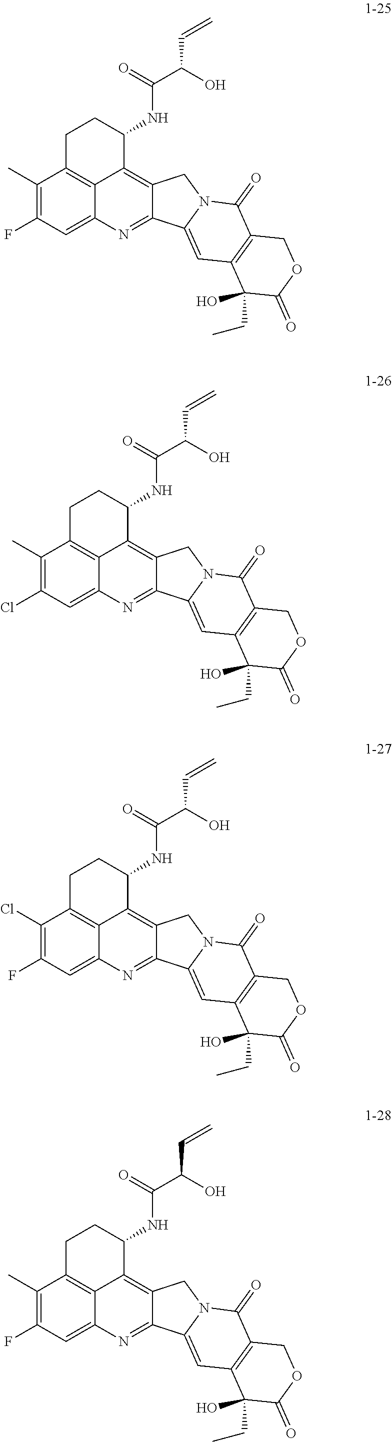 [SMILES: C=C[C@H](O)C(=O)N[C@H]1CCc2c(C)c(F)cc3nc4c(c1c23)Cn1c-4cc2c(c1=O)COC(=O)[C@]2(O)CC ; C=C[C@H](O)C(=O)N[C@H]1CCc2c(C)c(Cl)cc3nc4c(c1c23)Cn1c-4cc2c(c1=O)COC(=O)[C@]2(O)CC ; C=C[C@H](O)C(=O)N[C@H]1CCc2c(Cl)c(F)cc3nc4c(c1c23)Cn1c-4cc2c(c1=O)COC(=O)[C@]2(O)CC ; C=C[C@@H](O)C(=O)N[C@H]1CCc2c(C)c(F)cc3nc4c(c1c23)Cn1c-4cc2c(c1=O)COC(=O)[C@]2(O)CC] 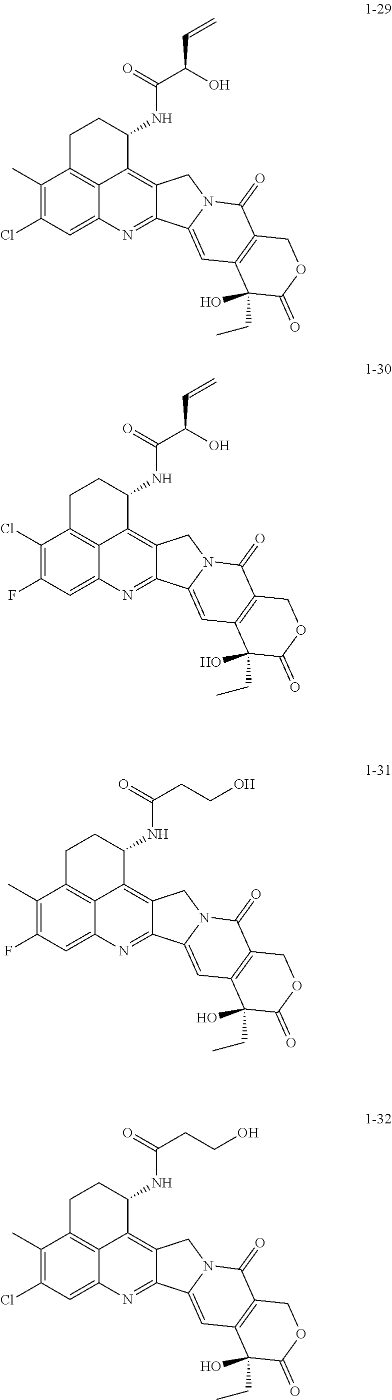 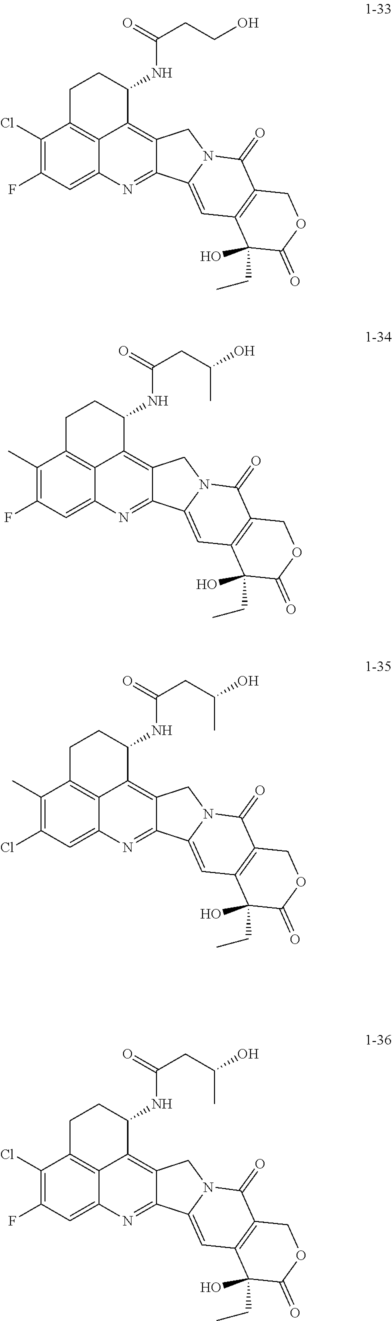 [SMILES: CC[C@@]1(O)C(=O)OCc2c1cc1n(c2=O)Cc2c-1nc1cc(F)c(Cl)c3c1c2[C@@H](NC(=O)CCO)CC3 ; CC[C@@]1(O)C(=O)OCc2c1cc1n(c2=O)Cc2c-1nc1cc(F)c(C)c3c1c2[C@@H](NC(=O)C[C@@H](C)O)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3] 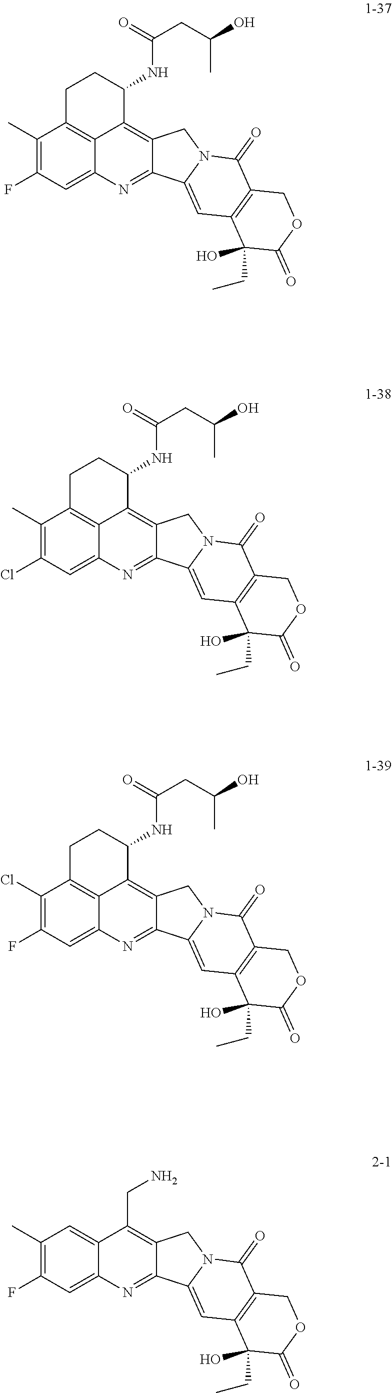 [SMILES: CC[C@@]1(O)C(=O)OCc2c1cc1n(c2=O)Cc2c-1nc1cc(F)c(C)c3c1c2[C@@H](NC(=O)C[C@H](C)O)CC3 ; CC[C@@]1(O)C(=O)OCc2c1cc1n(c2=O)Cc2c-1nc1cc(Cl)c(C)c3c1c2[C@@H](NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(F)c(C)cc1c2CN] 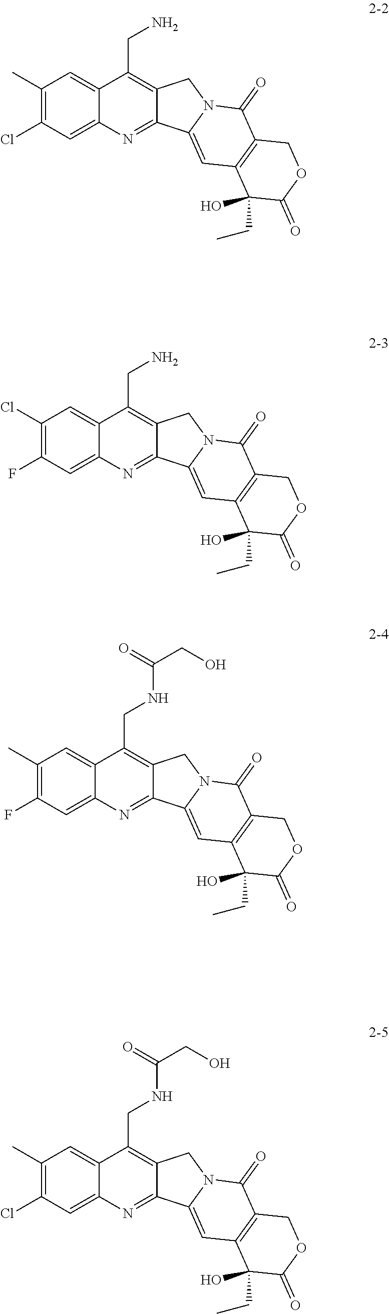 [SMILES: CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(F)c(C)cc1c2CNC(=O)CO ; CC[C@@]1(O)C(=O)OCc2c1cc1n(c2=O)Cc2c-1nc1cc(Cl)c(C)cc1c2CNC(=O)CO] 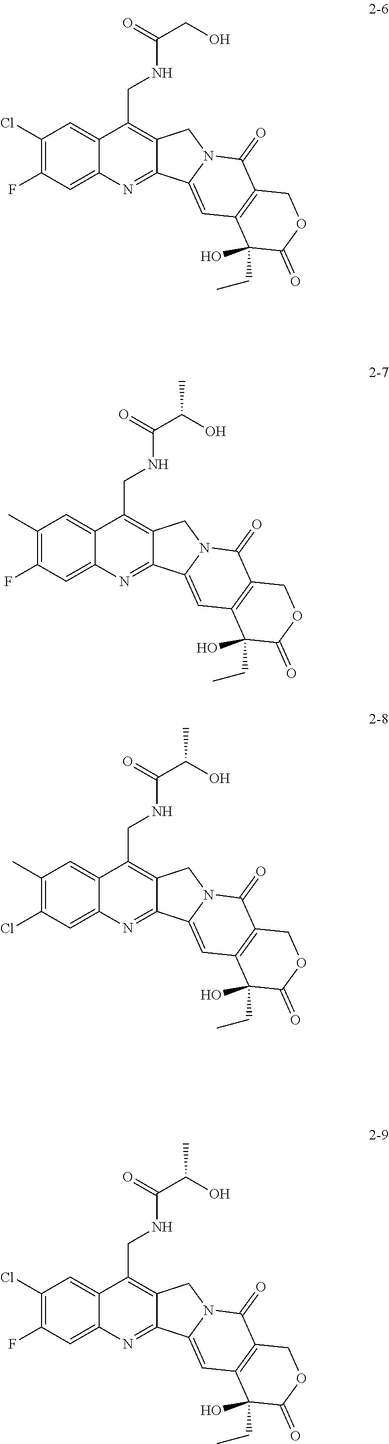 [SMILES: CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(F)c(C)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O] 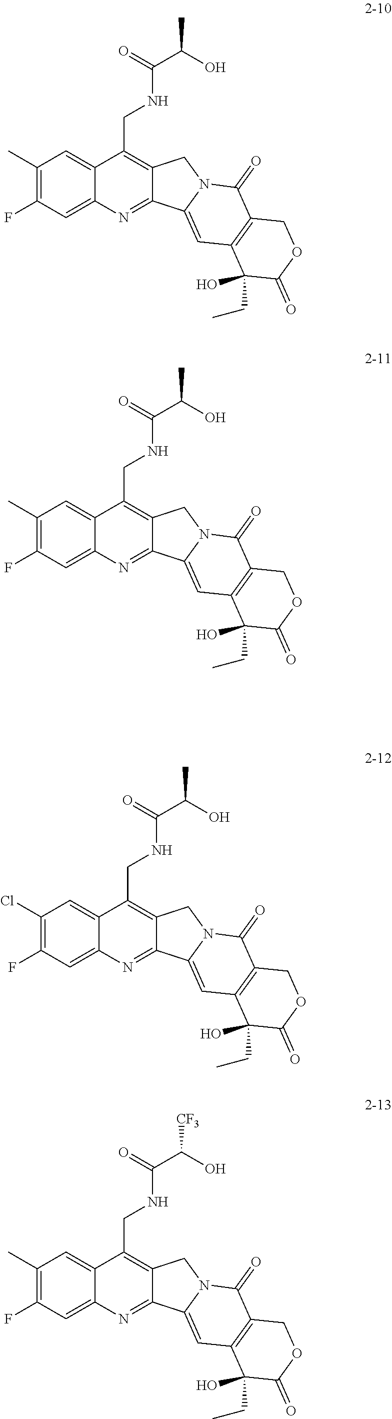 [SMILES: CC[C@@]1(O)C(=O)OCc2c1cc1n(c2=O)Cc2c-1nc1cc(F)c(C)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](O)C(F)(F)F] 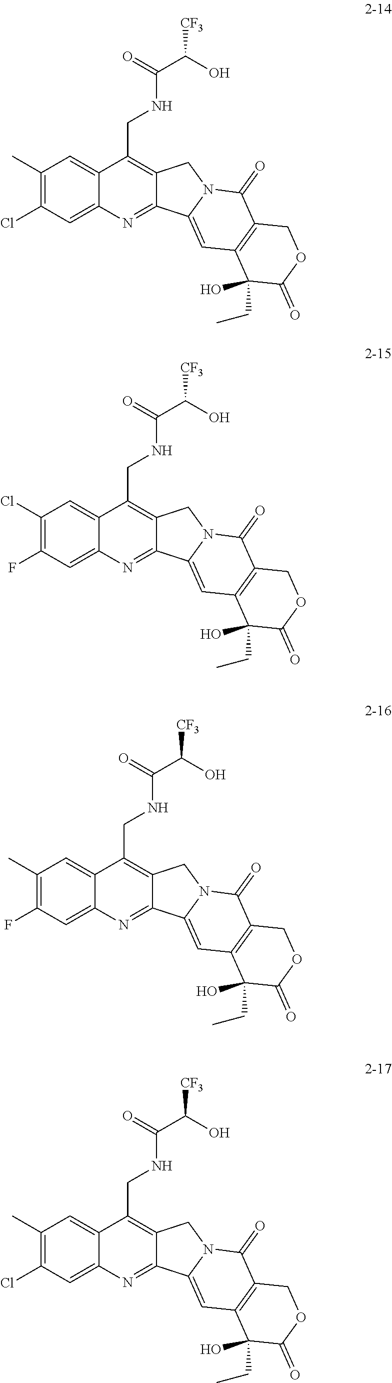 [SMILES: CC[C@@]1(O)C(=O)OCc2c1cc1n(c2=O)Cc2c-1nc1cc(Cl)c(C)cc1c2CNC(=O)[C@@H](O)C(F)(F)F ; CC[C@@]1(O)C(=O)OCc2c1cc1n(c2=O)Cc2c-1nc1cc(F)c(Cl)cc1c2CNC(=O)[C@@H](O)C(F)(F)F ; CC[C@@]1(O)C(=O)OCc2c1cc1n(c2=O)Cc2c-1nc1cc(F)c(C)cc1c2CNC(=O)[C@H](O)C(F)(F)F ; CC[C@@]1(O)C(=O)OCc2c1cc1n(c2=O)Cc2c-1nc1cc(Cl)c(C)cc1c2CNC(=O)[C@H](O)C(F)(F)F] 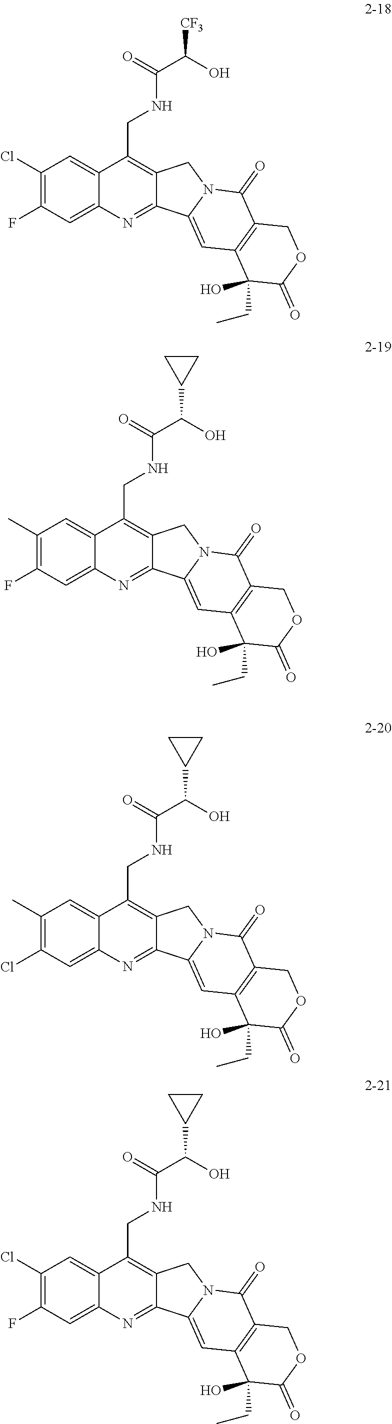 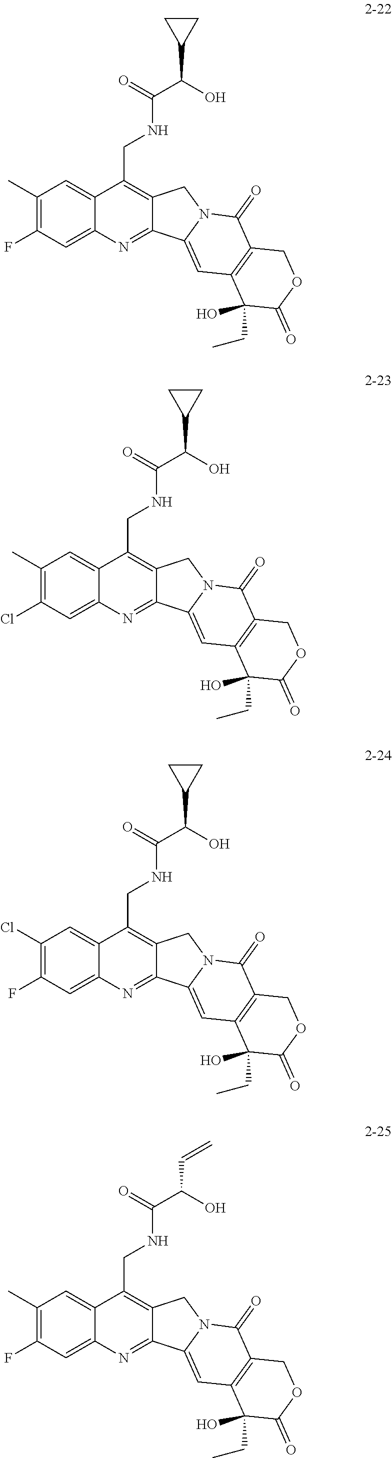 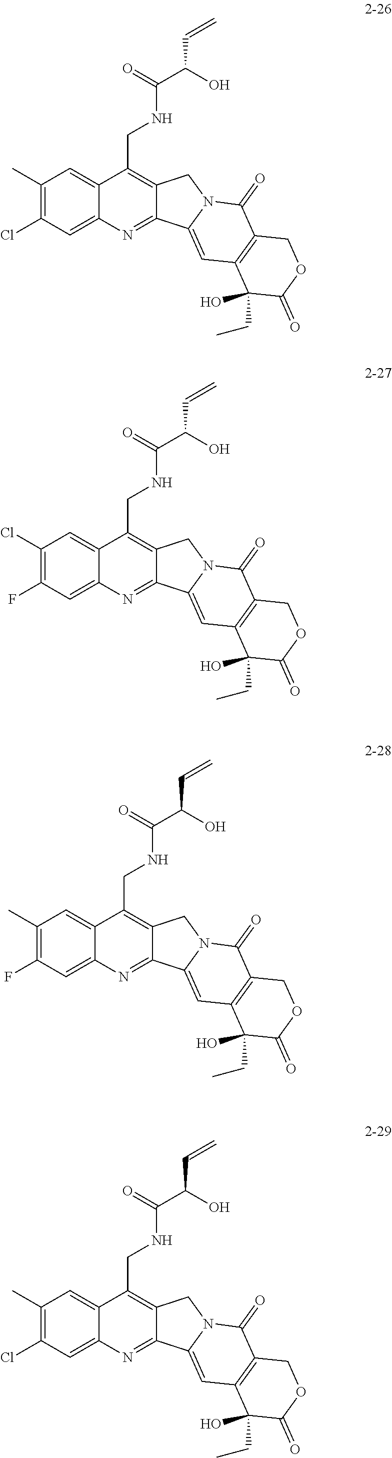 [SMILES: C=C[C@H](O)C(=O)NCc1c2c(nc3cc(Cl)c(C)cc13)-c1cc3c(c(=O)n1C2)COC(=O)[C@]3(O)CC ; C=C[C@H](O)C(=O)NCc1c2c(nc3cc(F)c(Cl)cc13)-c1cc3c(c(=O)n1C2)COC(=O)[C@]3(O)CC ; C=C[C@@H](O)C(=O)NCc1c2c(nc3cc(F)c(C)cc13)-c1cc3c(c(=O)n1C2)COC(=O)[C@]3(O)CC ; C=C[C@@H](O)C(=O)NCc1c2c(nc3cc(Cl)c(C)cc13)-c1cc3c(c(=O)n1C2)COC(=O)[C@]3(O)CC] 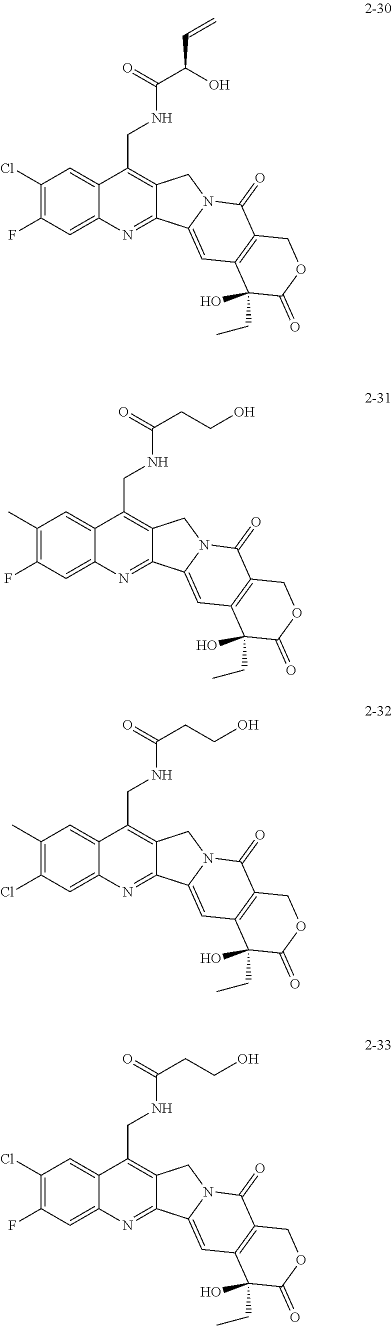 [SMILES: C=C[C@@H](O)C(=O)NCc1c2c(nc3cc(F)c(Cl)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2CNC(=O)CCO ; CC[C@@]1(O)C(=O)OCc2c1cc1n(c2=O)Cc2c-1nc1cc(Cl)c(C)cc1c2CNC(=O)CCO ; CC[C@@]1(O)C(=O)OCc2c1cc1n(c2=O)Cc2c-1nc1cc(F)c(Cl)cc1c2CNC(=O)CCO] 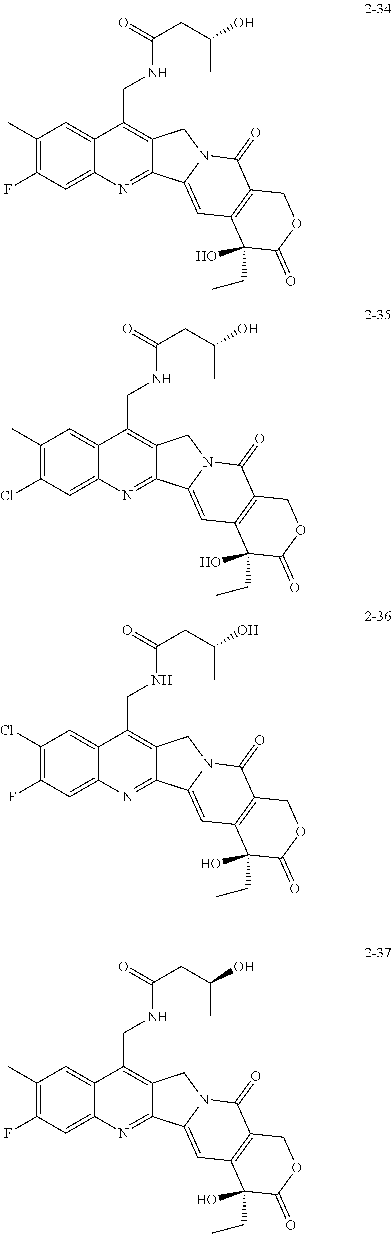 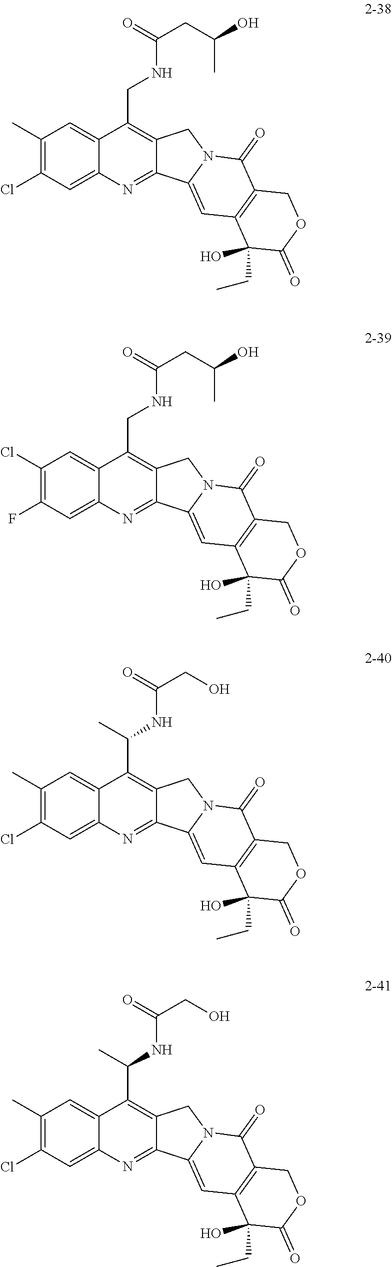 [SMILES: CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2[C@H](C)NC(=O)CO ; CC[C@@]1(O)C(=O)OCc2c1cc1n(c2=O)Cc2c-1nc1cc(Cl)c(C)cc1c2[C@@H](C)NC(=O)CO] 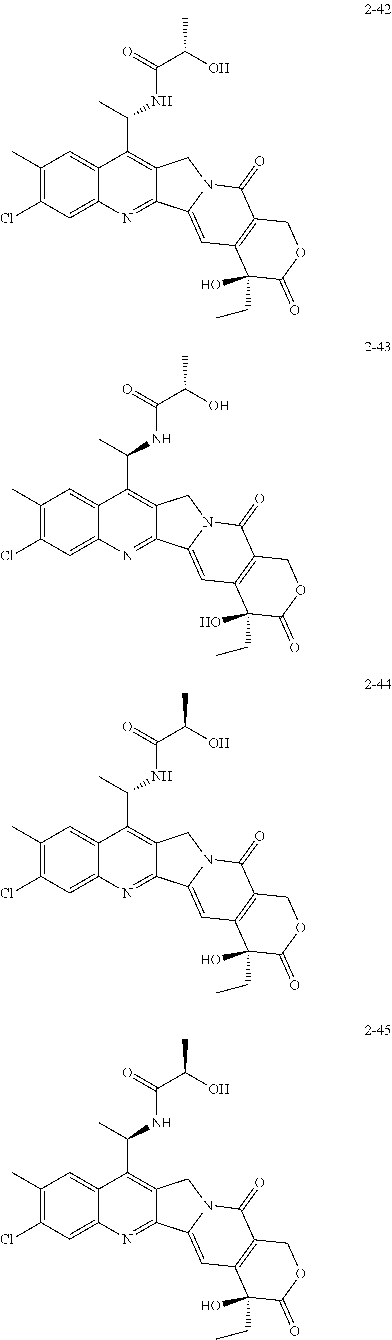 [SMILES: CC[C@@]1(O)C(=O)OCc2c1cc1n(c2=O)Cc2c-1nc1cc(Cl)c(C)cc1c2[C@H](C)NC(=O)[C@H](C)O ; CC[C@@]1(O)C(=O)OCc2c1cc1n(c2=O)Cc2c-1nc1cc(Cl)c(C)cc1c2[C@@H](C)NC(=O)[C@H](C)O ; CC[C@@]1(O)C(=O)OCc2c1cc1n(c2=O)Cc2c-1nc1cc(Cl)c(C)cc1c2[C@H](C)NC(=O)[C@@H](C)O ; CC[C@@]1(O)C(=O)OCc2c1cc1n(c2=O)Cc2c-1nc1cc(Cl)c(C)cc1c2[C@@H](C)NC(=O)[C@@H](C)O] 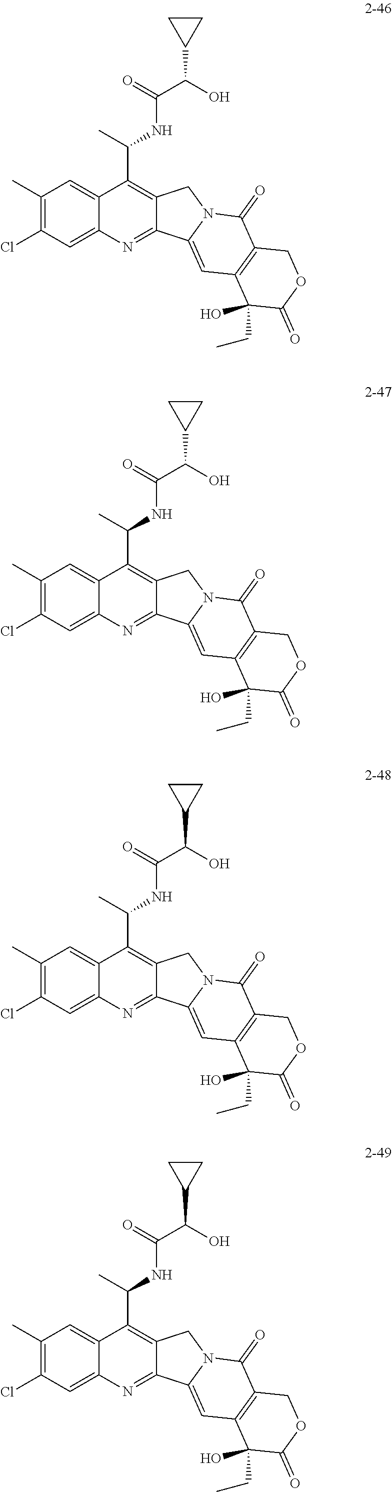 [SMILES: CC[C@@]1(O)C(=O)OCc2c1cc1n(c2=O)Cc2c-1nc1cc(Cl)c(C)cc1c2[C@H](C)NC(=O)[C@@H](O)C1CC1 ; CC[C@@]1(O)C(=O)OCc2c1cc1n(c2=O)Cc2c-1nc1cc(Cl)c(C)cc1c2[C@@H](C)NC(=O)[C@@H](O)C1CC1 ; CC[C@@]1(O)C(=O)OCc2c1cc1n(c2=O)Cc2c-1nc1cc(Cl)c(C)cc1c2[C@H](C)NC(=O)[C@H](O)C1CC1 ; CC[C@@]1(O)C(=O)OCc2c1cc1n(c2=O)Cc2c-1nc1cc(Cl)c(C)cc1c2[C@@H](C)NC(=O)[C@H](O)C1CC1] 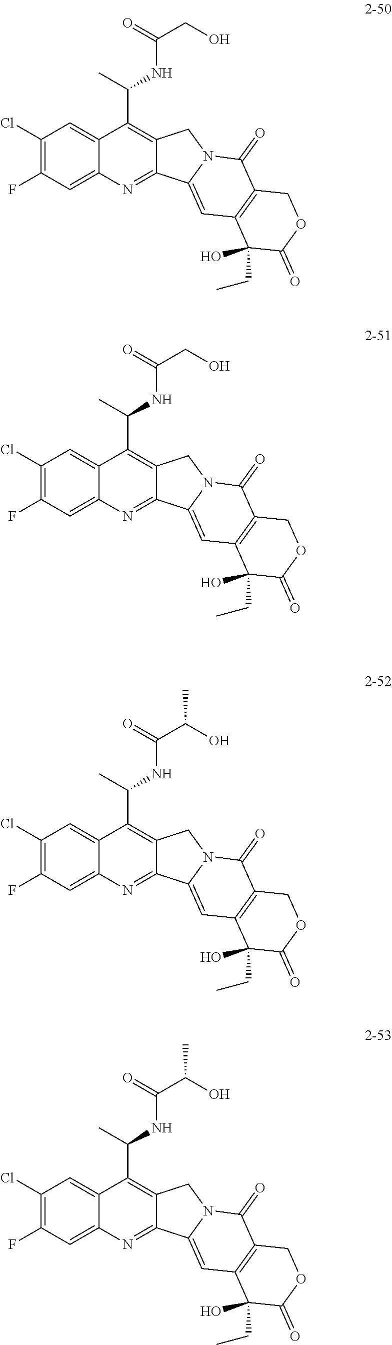 [SMILES: CC[C@@]1(O)C(=O)OCc2c1cc1n(c2=O)Cc2c-1nc1cc(F)c(Cl)cc1c2[C@H](C)NC(=O)CO ; CC[C@@]1(O)C(=O)OCc2c1cc1n(c2=O)Cc2c-1nc1cc(F)c(Cl)cc1c2[C@@H](C)NC(=O)CO ; CC[C@@]1(O)C(=O)OCc2c1cc1n(c2=O)Cc2c-1nc1cc(F)c(Cl)cc1c2[C@H](C)NC(=O)[C@H](C)O ; CC[C@@]1(O)C(=O)OCc2c1cc1n(c2=O)Cc2c-1nc1cc(F)c(Cl)cc1c2[C@@H](C)NC(=O)[C@H](C)O] 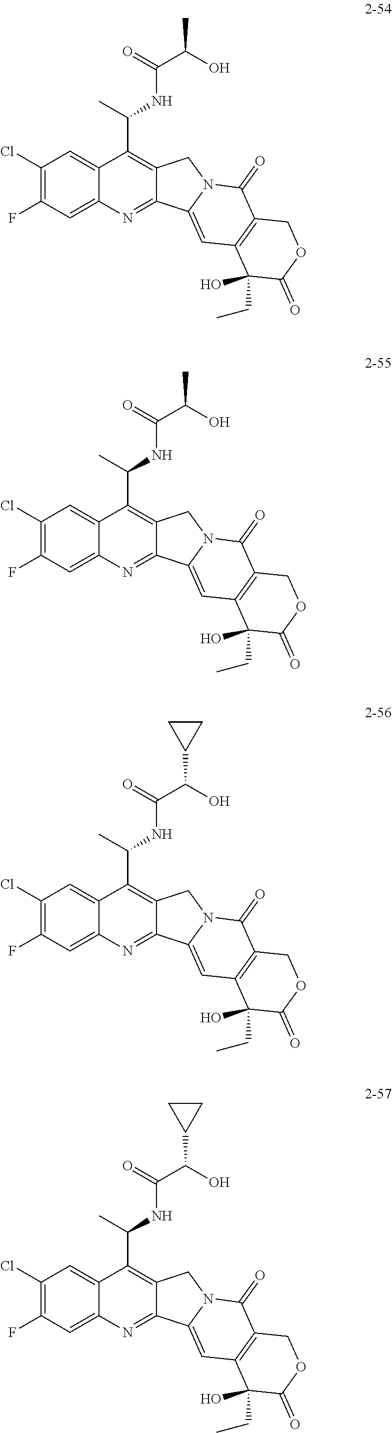 [SMILES: CC[C@@]1(O)C(=O)OCc2c1cc1n(c2=O)Cc2c-1nc1cc(F)c(Cl)cc1c2[C@H](C)NC(=O)[C@@H](C)O ; CC[C@@]1(O)C(=O)OCc2c1cc1n(c2=O)Cc2c-1nc1cc(F)c(Cl)cc1c2[C@@H](C)NC(=O)[C@@H](C)O ; CC[C@@]1(O)C(=O)OCc2c1cc1n(c2=O)Cc2c-1nc1cc(F)c(Cl)cc1c2[C@H](C)NC(=O)[C@@H](O)C1CC1 ; CC[C@@]1(O)C(=O)OCc2c1cc1n(c2=O)Cc2c-1nc1cc(F)c(Cl)cc1c2[C@@H](C)NC(=O)[C@@H](O)C1CC1] 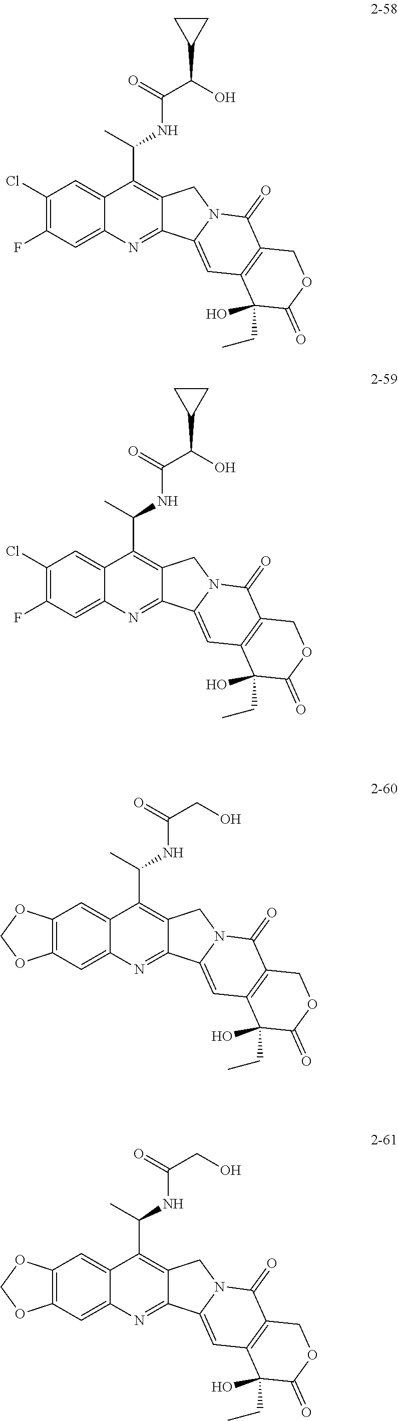 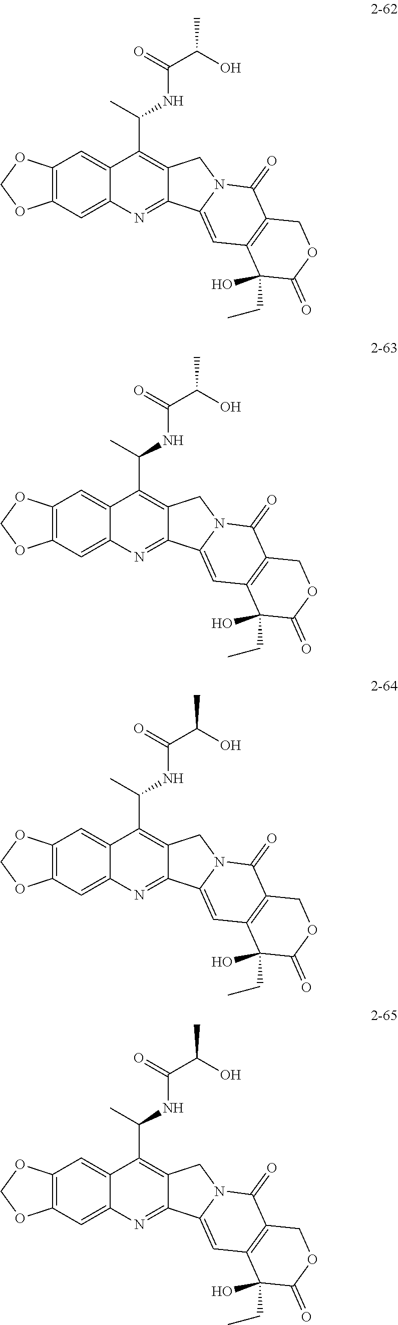 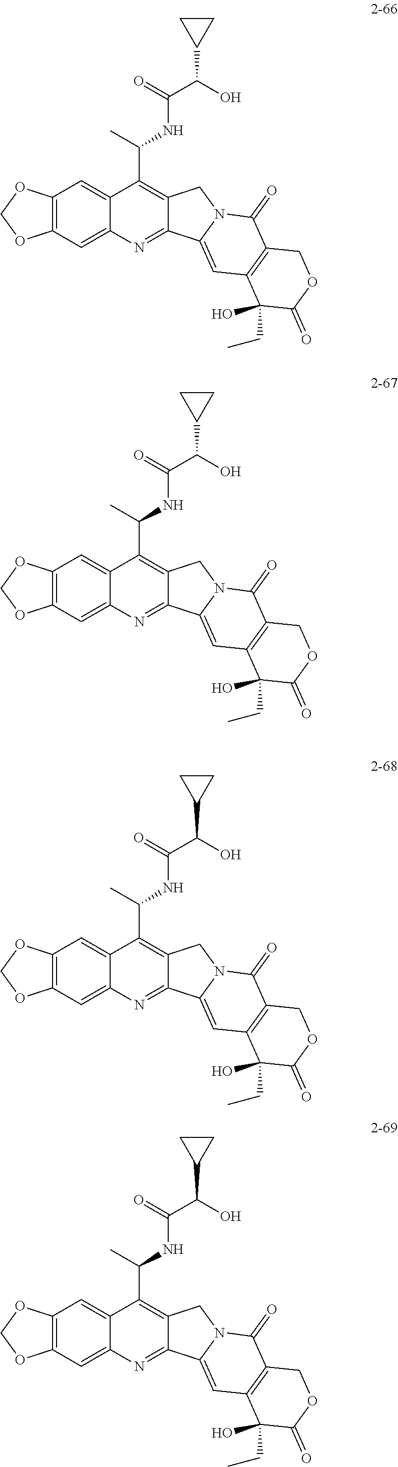 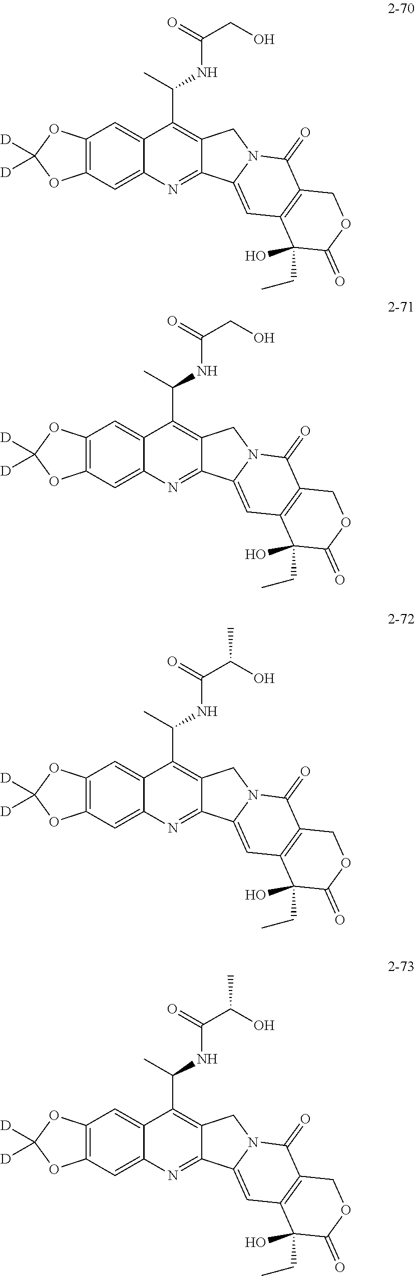 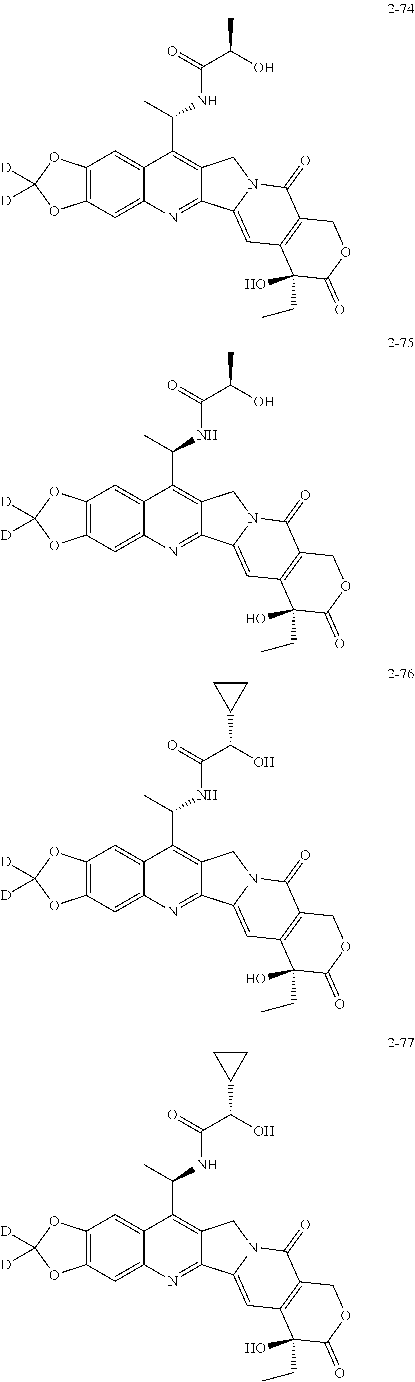 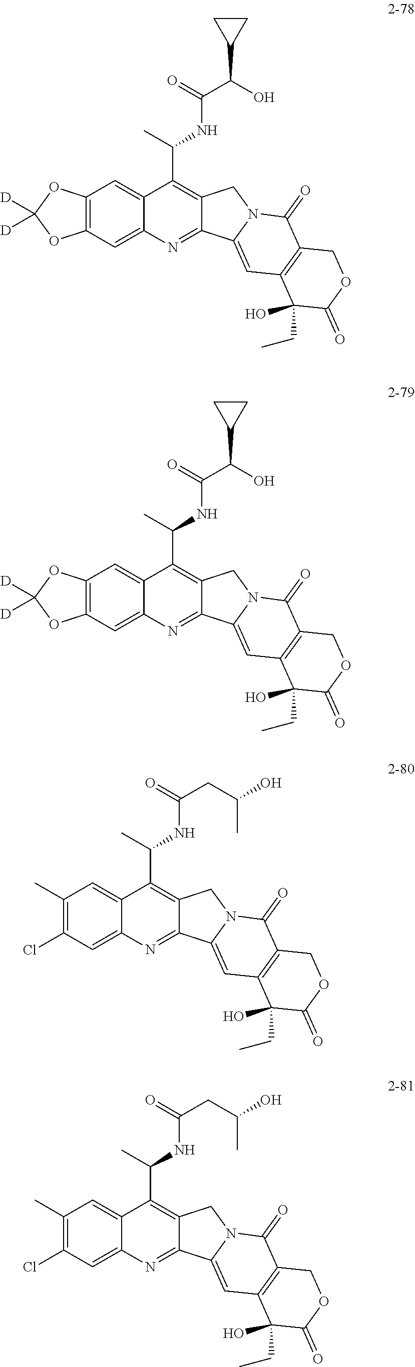 [SMILES: [2H]C1([2H])Oc2cc3nc4c(c([C@H](C)NC(=O)[C@H](O)C5CC5)c3cc2O1)Cn1c-4cc2c(c1=O)COC(=O)[C@]2(O)CC ; [2H]C1([2H])Oc2cc3nc4c(c([C@@H](C)NC(=O)[C@H](O)C5CC5)c3cc2O1)Cn1c-4cc2c(c1=O)COC(=O)[C@]2(O)CC ; CC[C@@]1(O)C(=O)OCc2c1cc1n(c2=O)Cc2c-1nc1cc(Cl)c(C)cc1c2[C@H](C)NC(=O)C[C@@H](C)O ; CC[C@@]1(O)C(=O)OCc2c1cc1n(c2=O)Cc2c-1nc1cc(Cl)c(C)cc1c2[C@@H](C)NC(=O)C[C@@H](C)O] 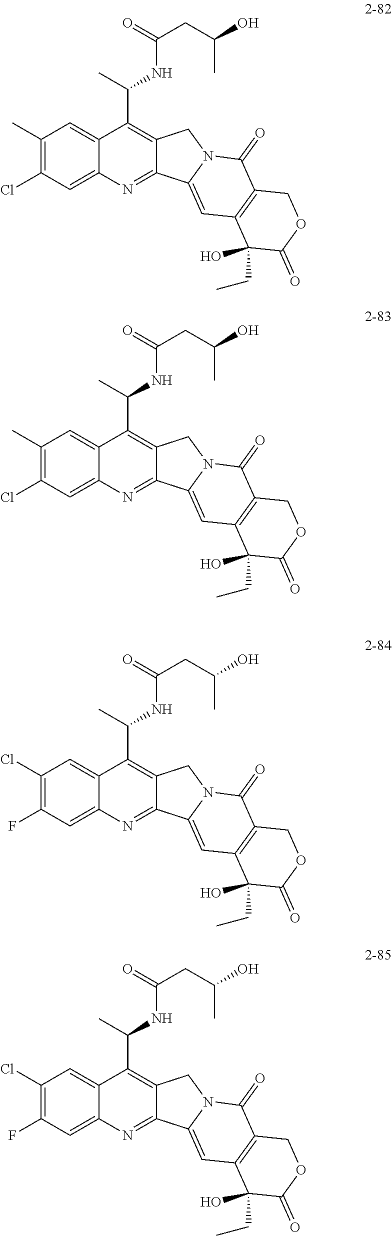 [SMILES: CC[C@@]1(O)C(=O)OCc2c1cc1n(c2=O)Cc2c-1nc1cc(Cl)c(C)cc1c2[C@H](C)NC(=O)C[C@H](C)O ; CC[C@@]1(O)C(=O)OCc2c1cc1n(c2=O)Cc2c-1nc1cc(Cl)c(C)cc1c2[C@@H](C)NC(=O)C[C@H](C)O ; CC[C@@]1(O)C(=O)OCc2c1cc1n(c2=O)Cc2c-1nc1cc(F)c(Cl)cc1c2[C@H](C)NC(=O)C[C@@H](C)O ; CC[C@@]1(O)C(=O)OCc2c1cc1n(c2=O)Cc2c-1nc1cc(F)c(Cl)cc1c2[C@@H](C)NC(=O)C[C@@H](C)O] 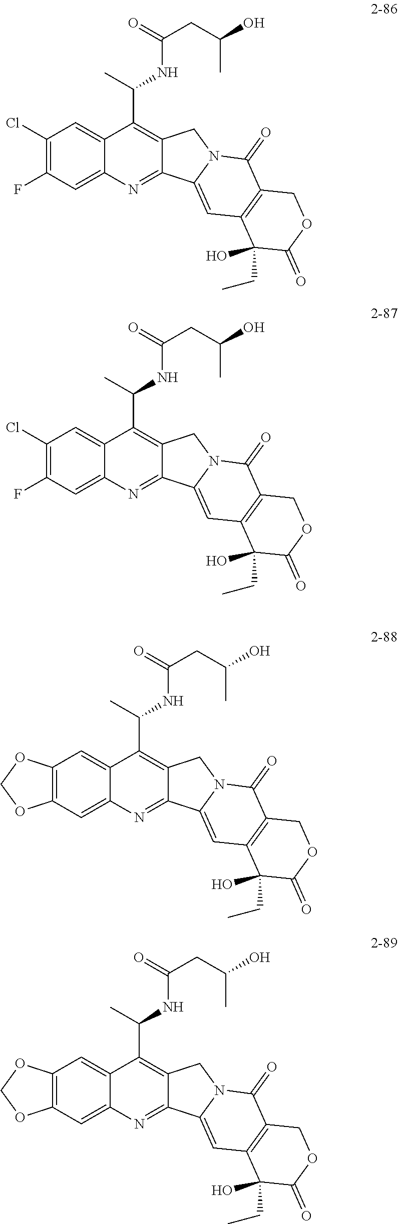 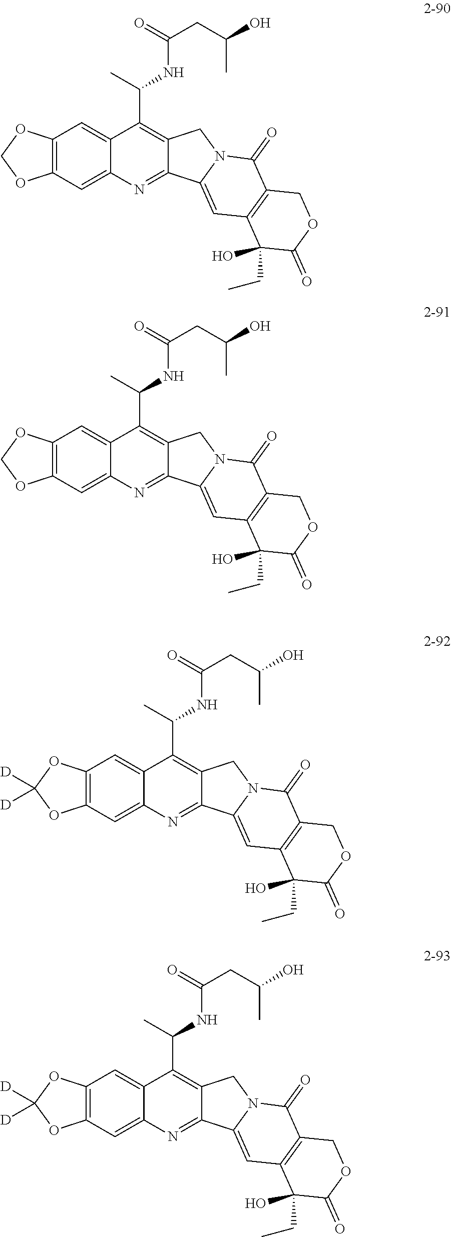 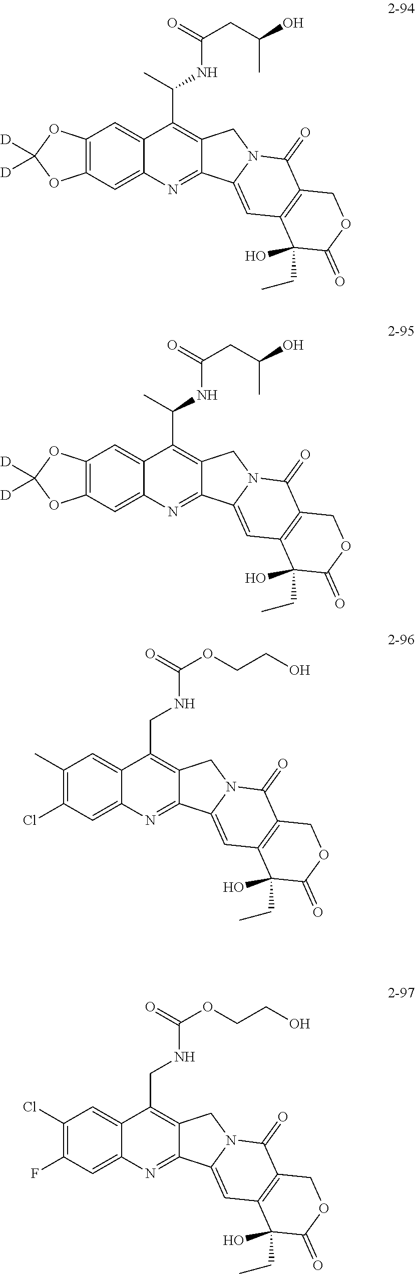 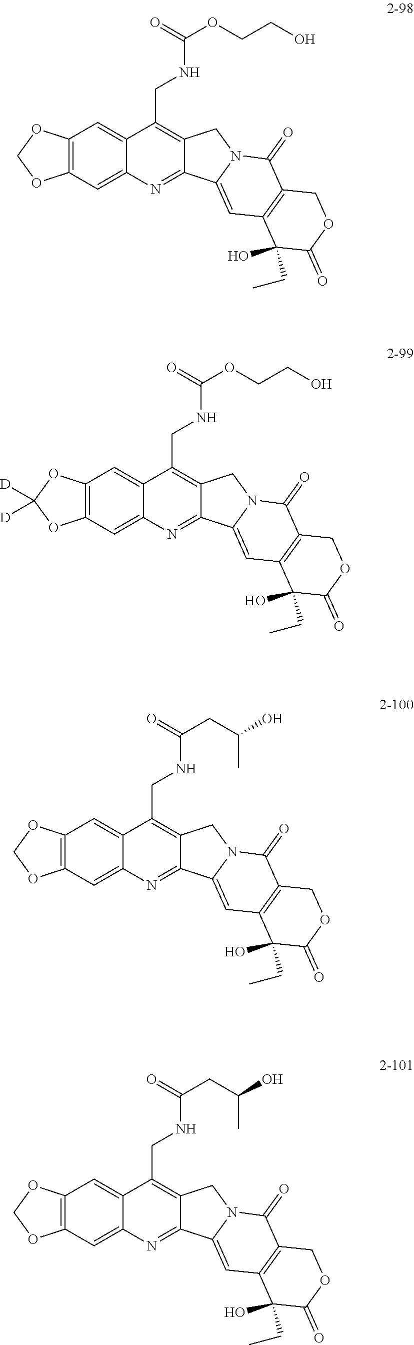 [SMILES: CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC ; CC[C@@]1(O)C(=O)OCc2c1cc1n(c2=O)Cc2c-1nc1cc3c(cc1c2CNC(=O)C[C@@H](C)O)OCO3 ; CC[C@@]1(O)C(=O)OCc2c1cc1n(c2=O)Cc2c-1nc1cc3c(cc1c2CNC(=O)C[C@H](C)O)OCO3] 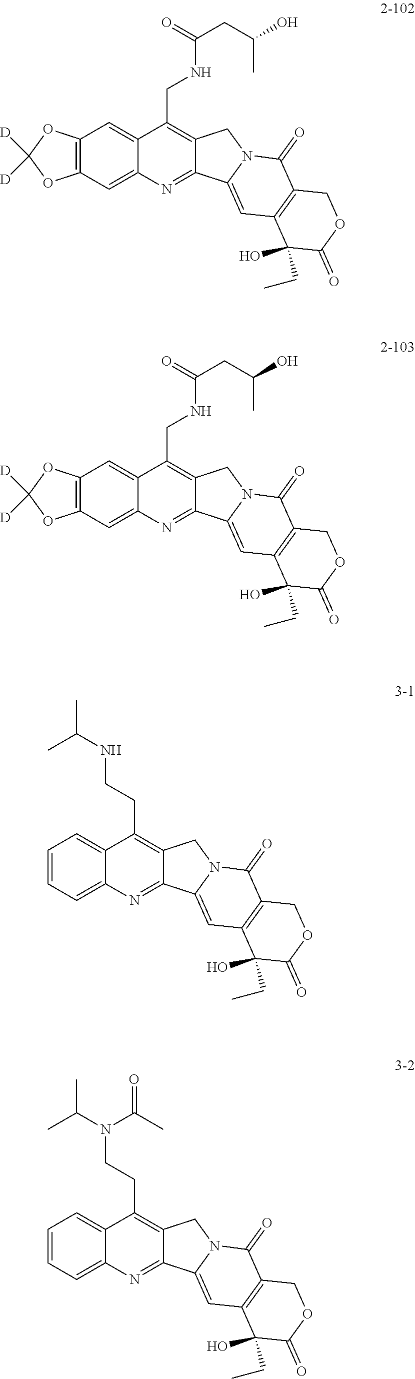 [SMILES: [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C] 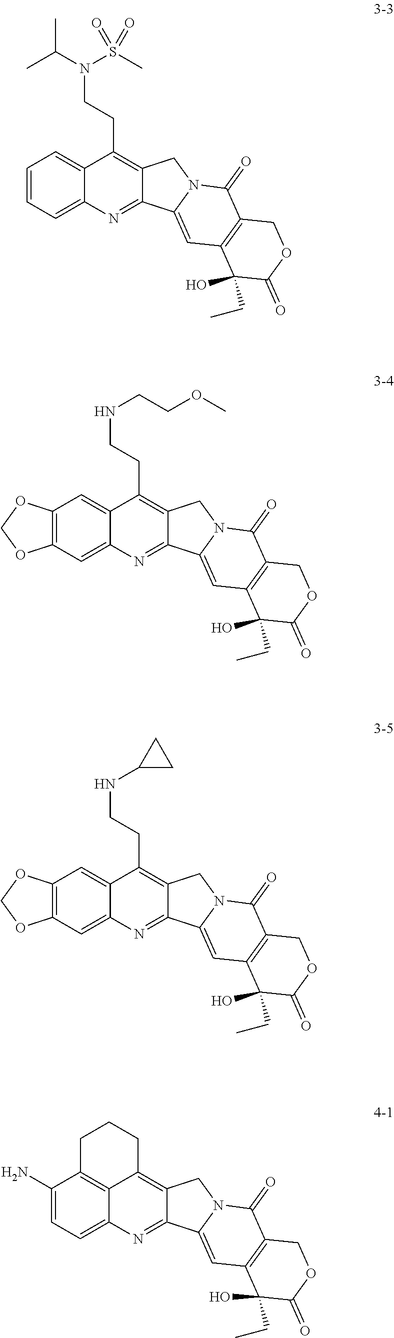 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1cc3c(cc1c2CCNC1CC1)OCO3 ; CC[C@@]1(O)C(=O)OCc2c1cc1n(c2=O)Cc2c-1nc1ccc(N)c3c1c2CCC3] 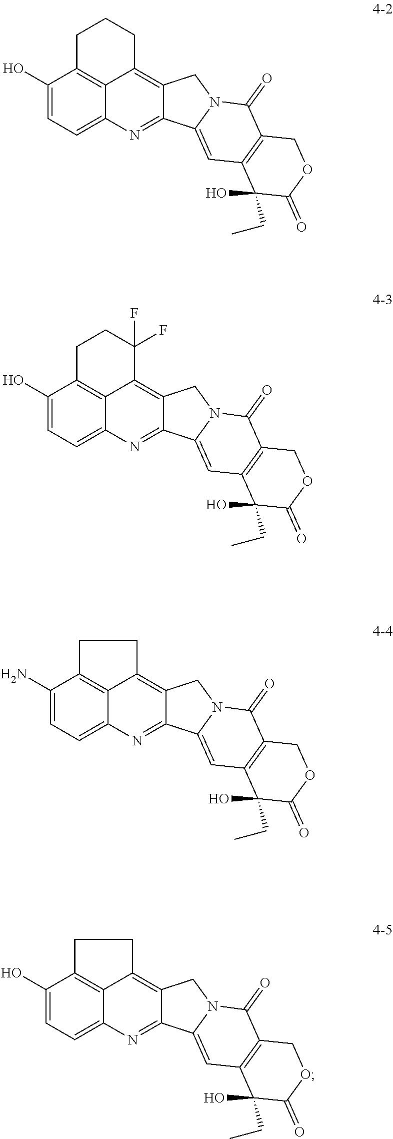 [SMILES: CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2C(F)(F)CC3 ; CC[C@@]1(O)C(=O)OCc2c1cc1n(c2=O)Cc2c-1nc1ccc(N)c3c1c2CC3 ; CC[C@@]1(O)C(=O)OCc2c1cc1n(c2=O)Cc2c-1nc1ccc(O)c3c1c2CC3]
preferably, the cytotoxic drug is selected from the group consisting of the following compounds or isotopically labeled compounds thereof: 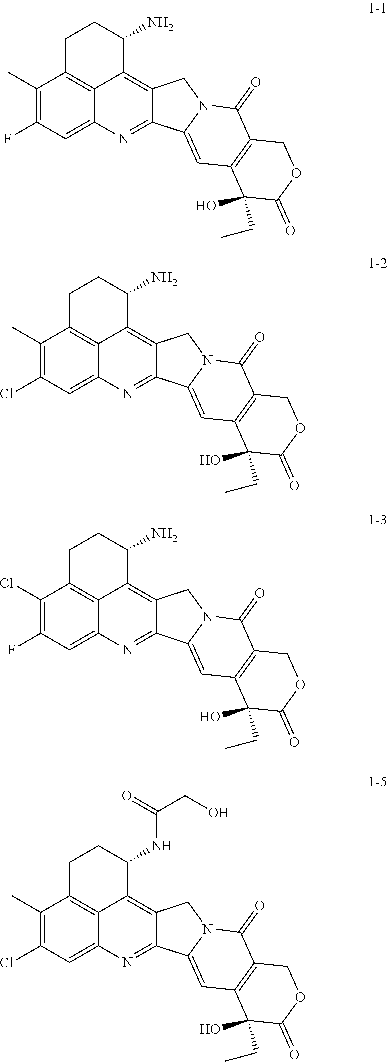 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3] 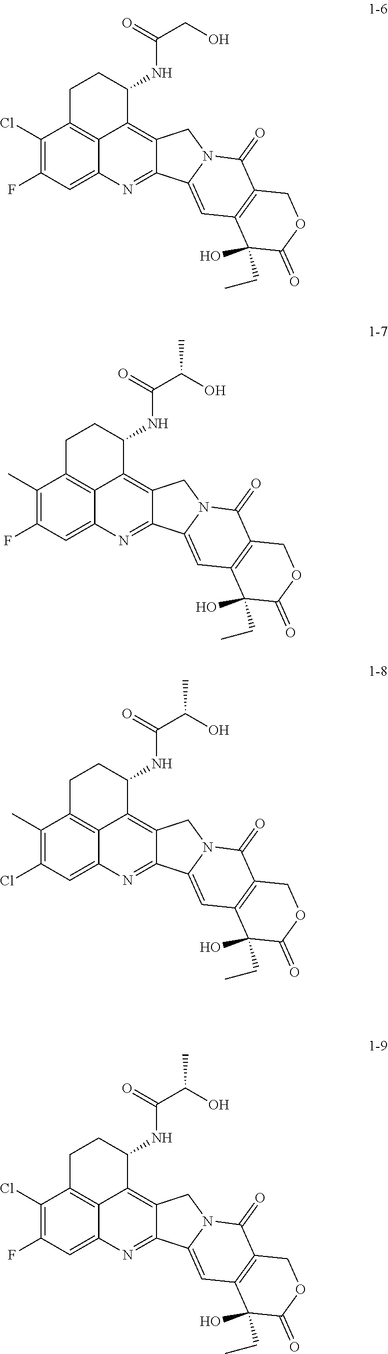 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3] 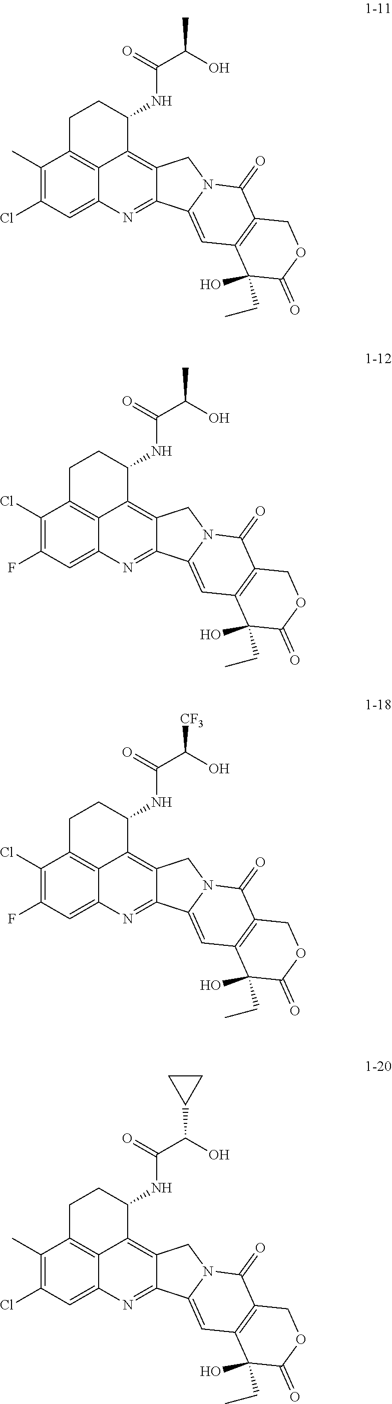 [SMILES: CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(Cl)c(C)c3c1c2[C@@H](NC(=O)[C@@H](O)C1CC1)CC3] 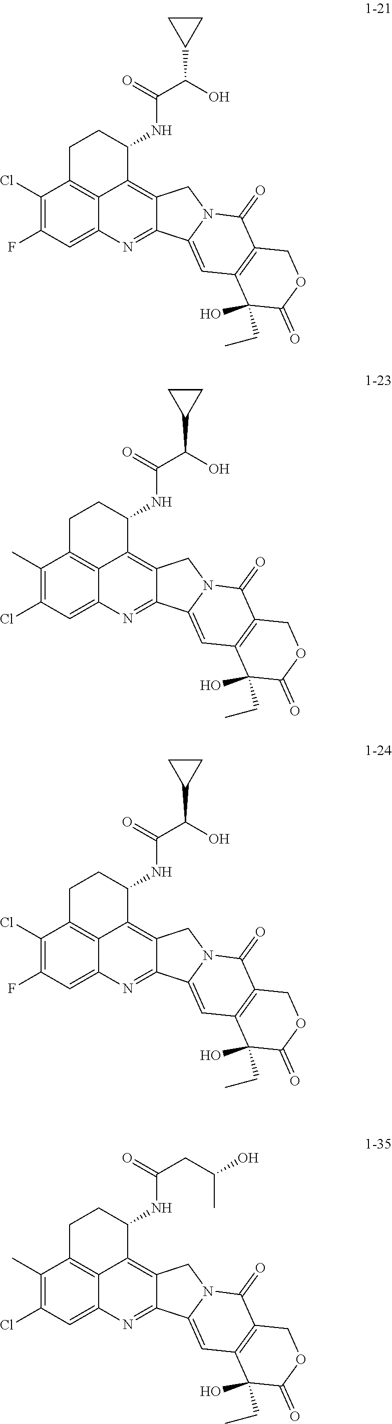 [SMILES: CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3] 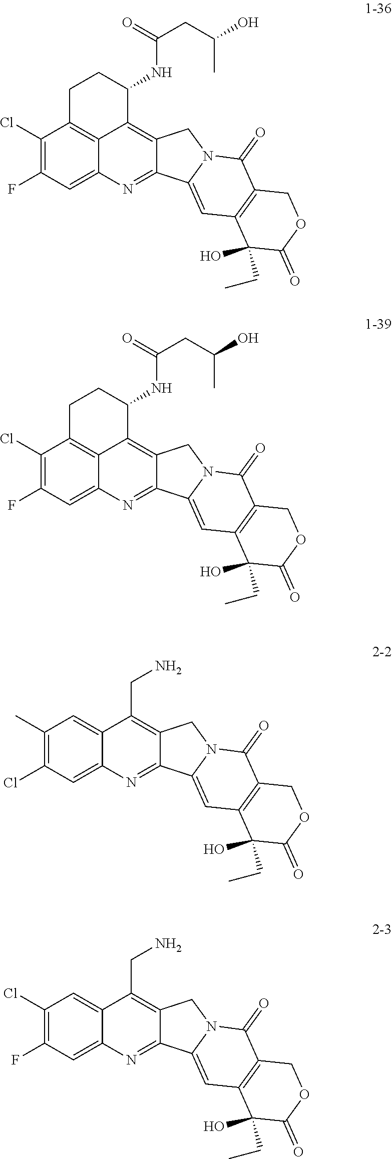 [SMILES: CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN] 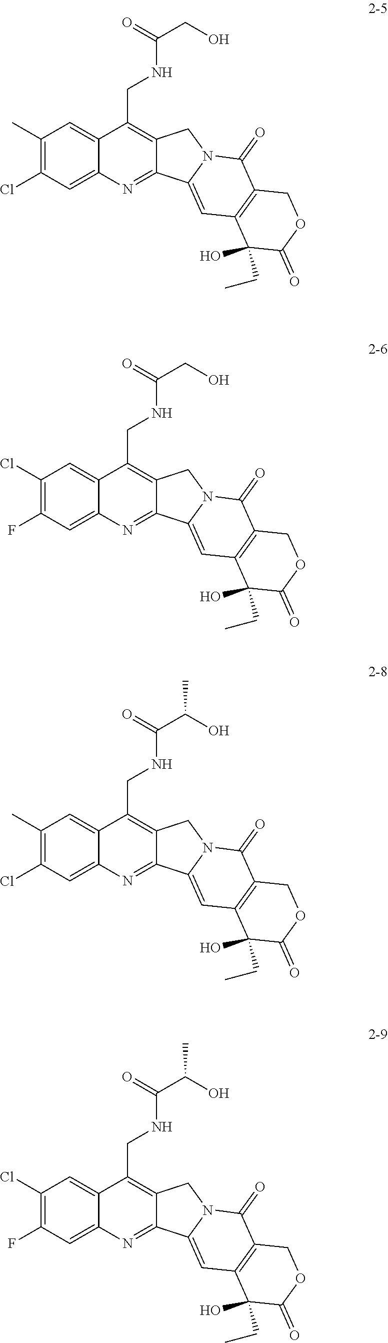 [SMILES: CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O] 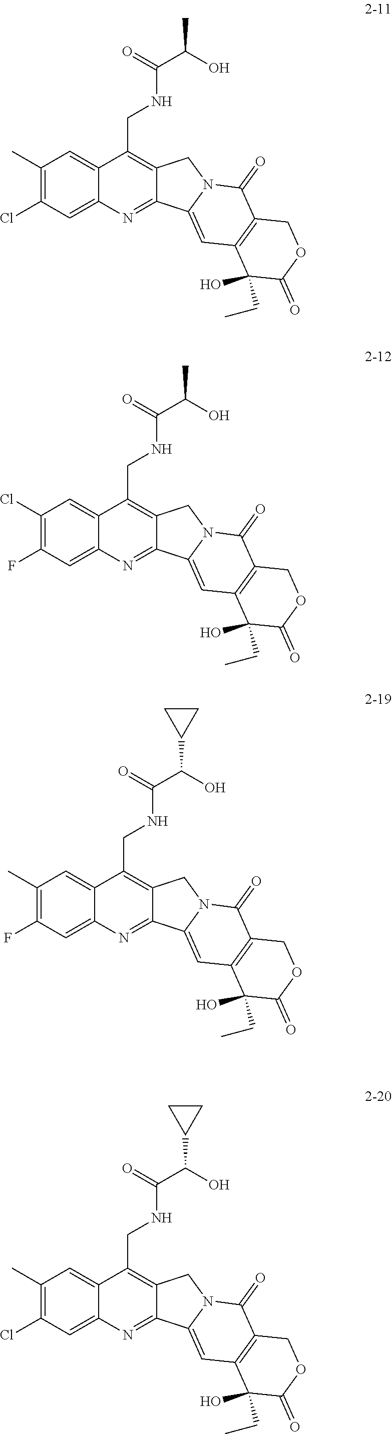 [SMILES: CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](O)C1CC1 ; CC[C@@]1(O)C(=O)OCc2c1cc1n(c2=O)Cc2c-1nc1cc(Cl)c(C)cc1c2CNC(=O)[C@@H](O)C1CC1] 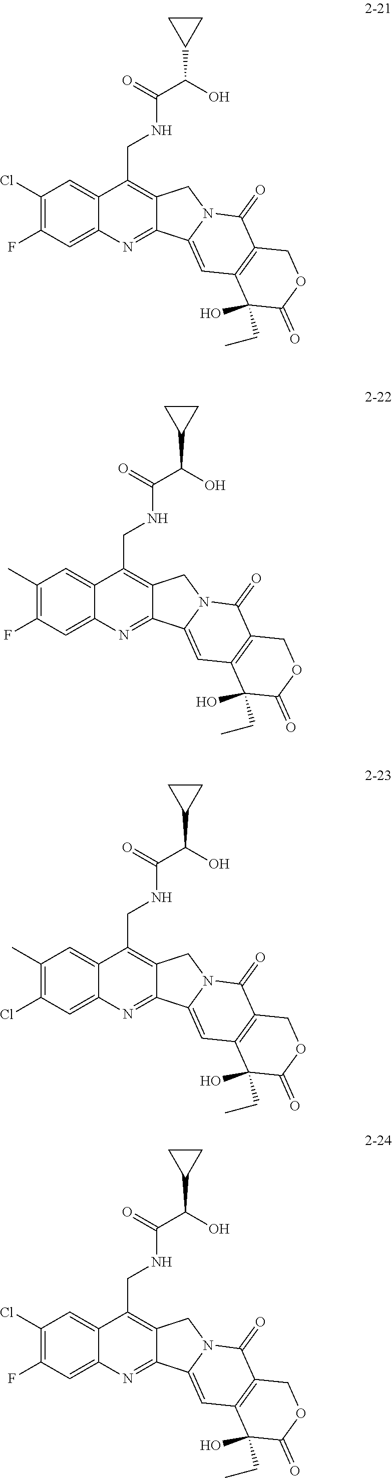 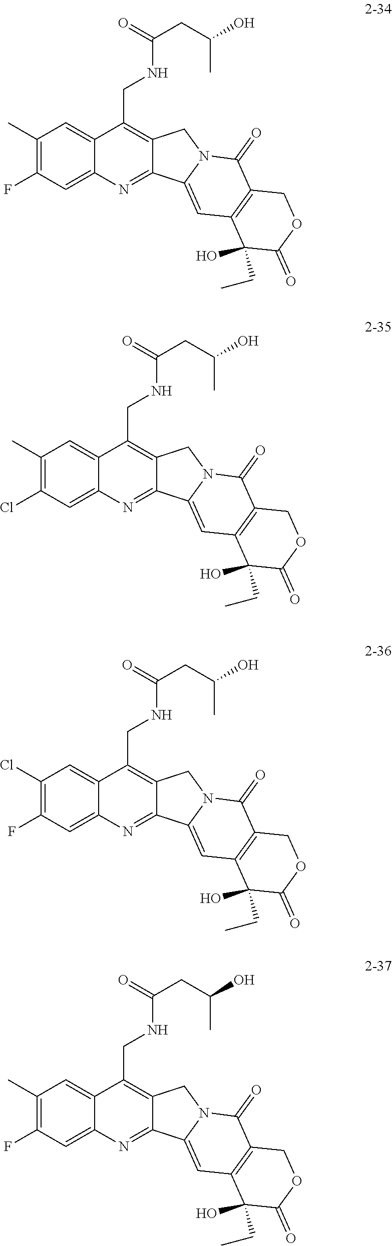 [SMILES: CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O] 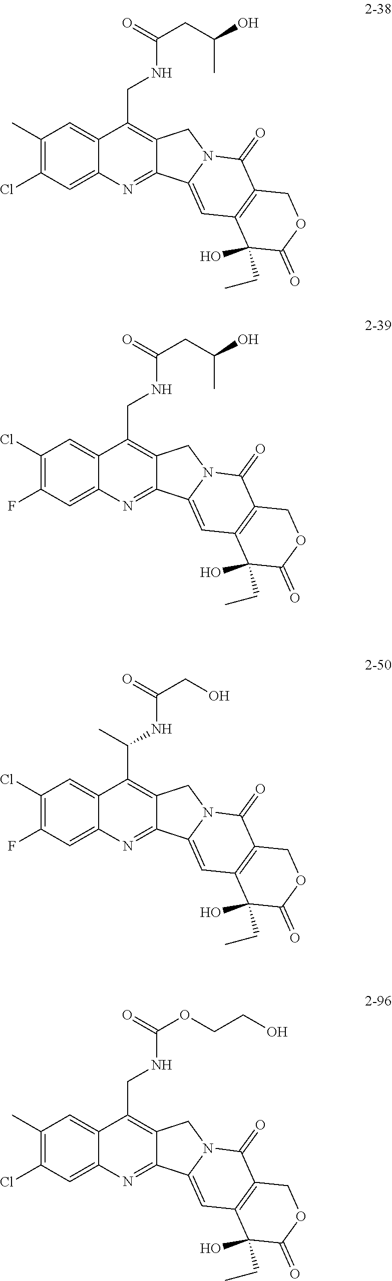 [SMILES: CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(F)c(Cl)cc1c2[C@H](C)NC(=O)CO ; CC[C@@]1(O)C(=O)OCc2c1cc1n(c2=O)Cc2c-1nc1cc(Cl)c(C)cc1c2CNC(=O)OCCO] 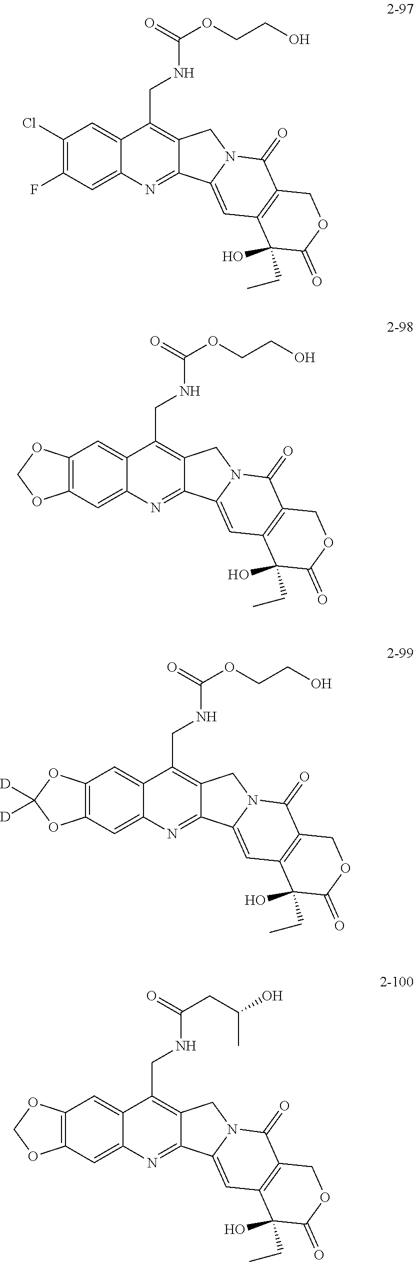 [SMILES: CC[C@@]1(O)C(=O)OCc2c1cc1n(c2=O)Cc2c-1nc1cc(F)c(Cl)cc1c2CNC(=O)OCCO ; CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC ; CC[C@@]1(O)C(=O)OCc2c1cc1n(c2=O)Cc2c-1nc1cc3c(cc1c2CNC(=O)C[C@@H](C)O)OCO3] 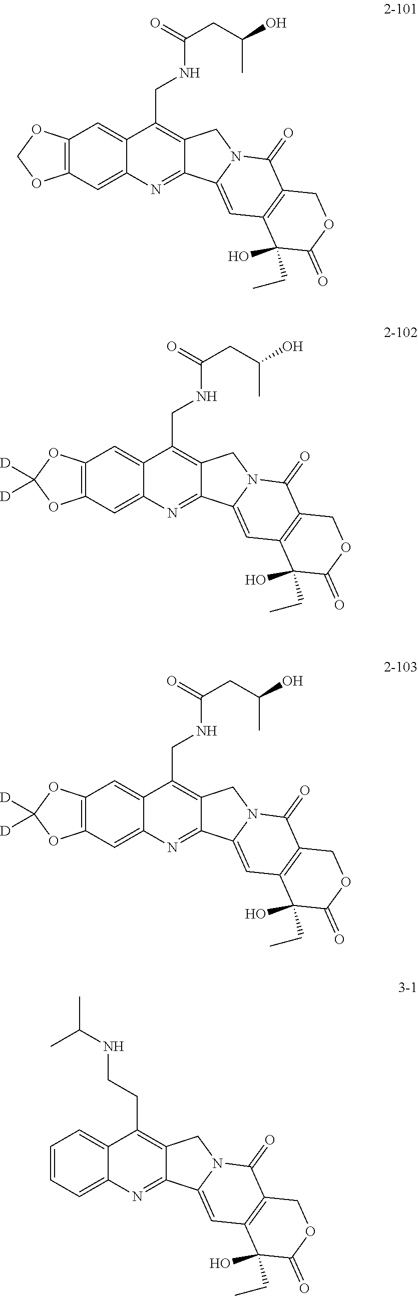 [SMILES: CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C] 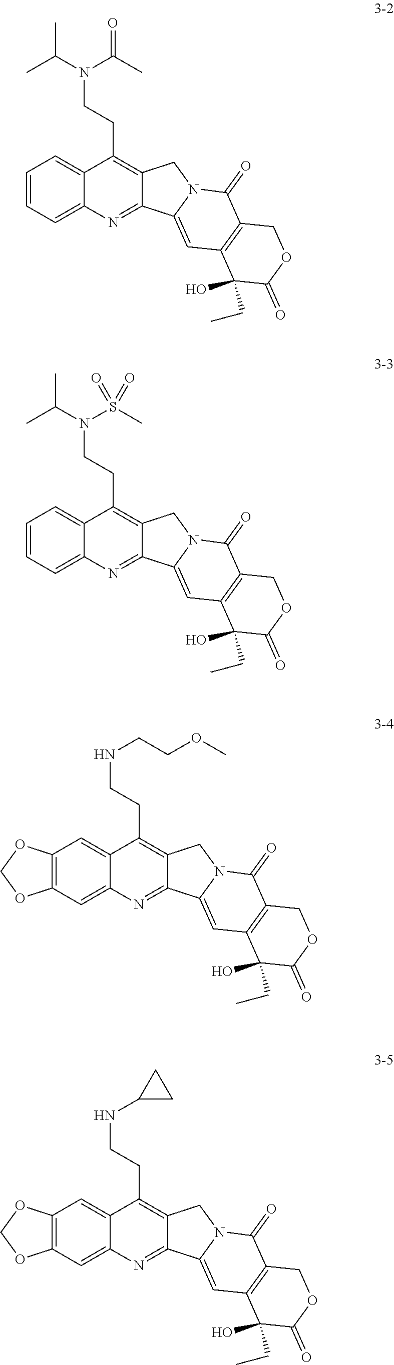 [SMILES: CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1cc3c(cc1c2CCNC1CC1)OCO3] 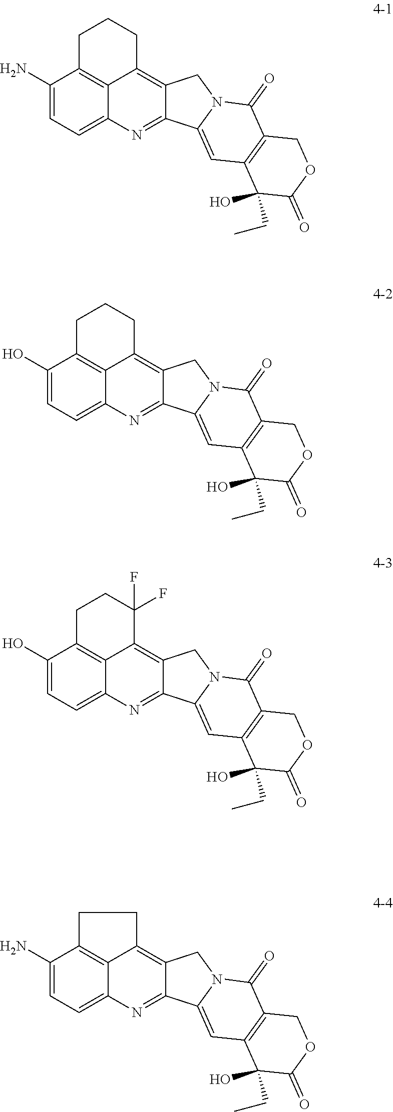 [SMILES: CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2C(F)(F)CC3 ; CC[C@@]1(O)C(=O)OCc2c1cc1n(c2=O)Cc2c-1nc1ccc(N)c3c1c2CC3] 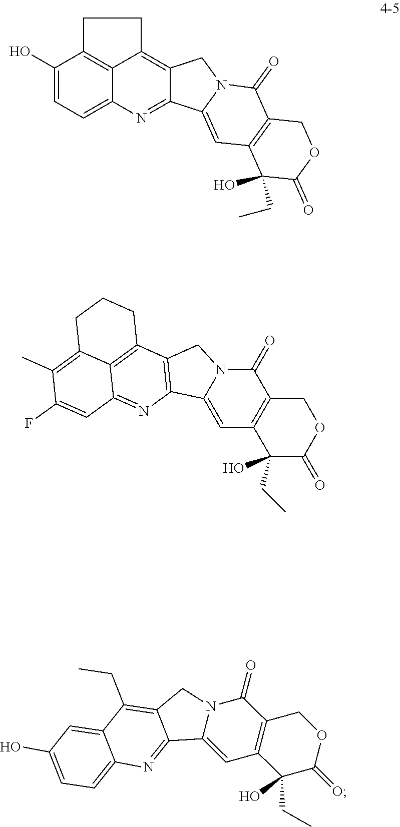 [SMILES: CC[C@@]1(O)C(=O)OCc2c1cc1n(c2=O)Cc2c-1nc1ccc(O)c3c1c2CC3 ; CC[C@@]1(O)C(=O)OCc2c1cc1n(c2=O)Cc2c-1nc1cc(F)c(C)c3c1c2CCC3 ; CCc1c2c(nc3ccc(O)cc13)-c1cc3c(c(=O)n1C2)COC(=O)[C@]3(O)CC]
preferably, the cytotoxic drug is selected from the group consisting of the following compounds or isotopically labeled compounds thereof: 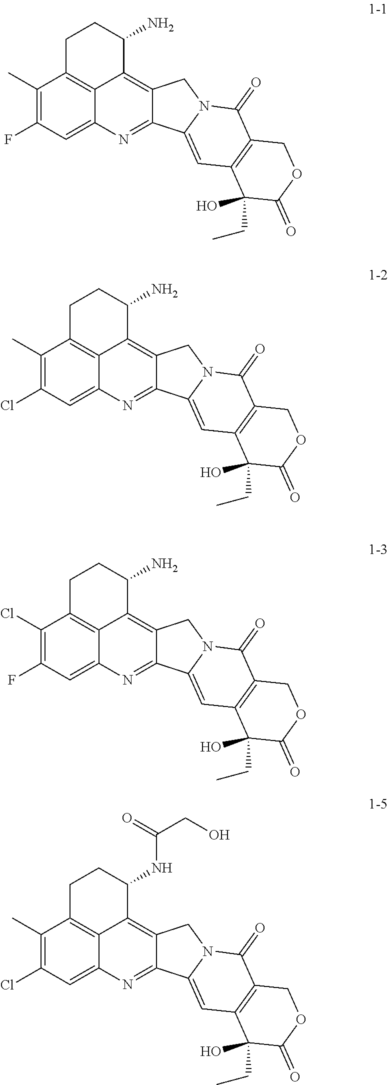 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3] 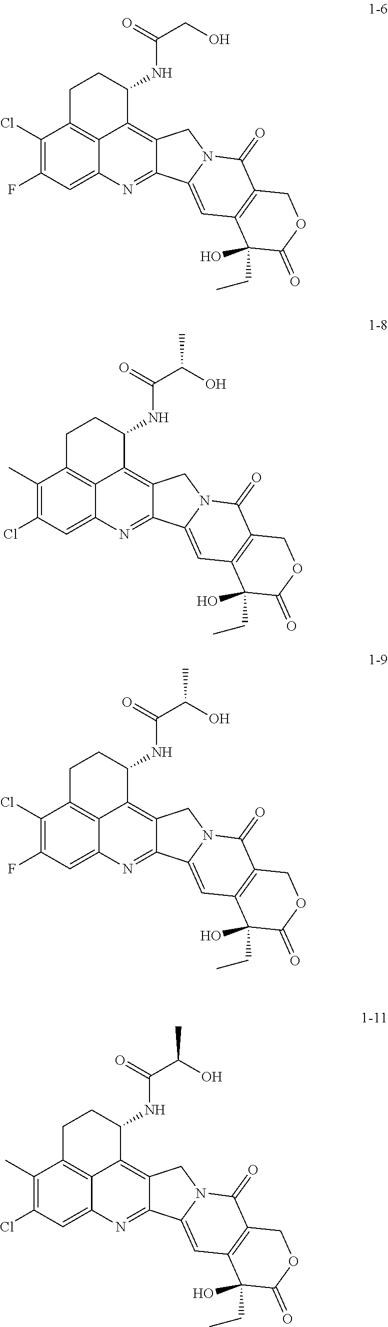 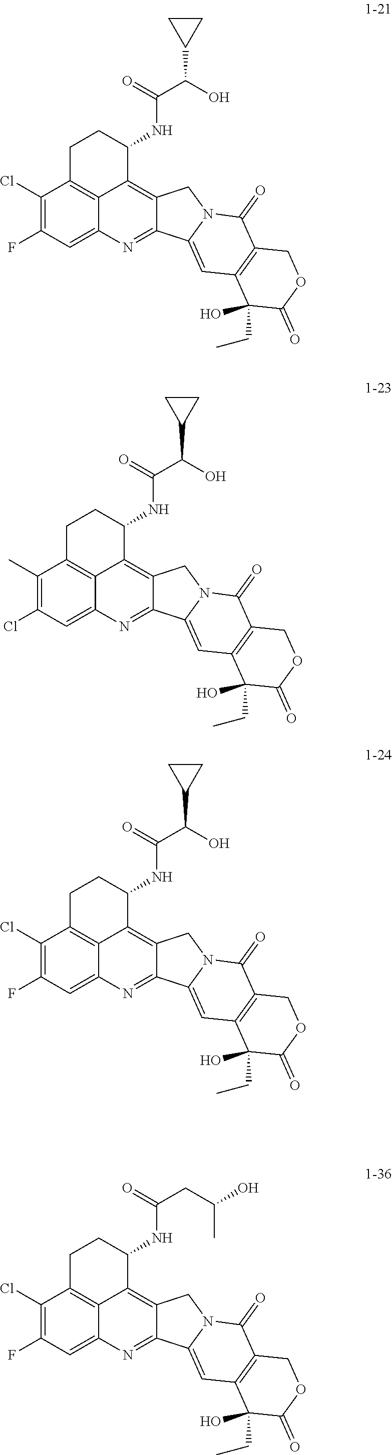 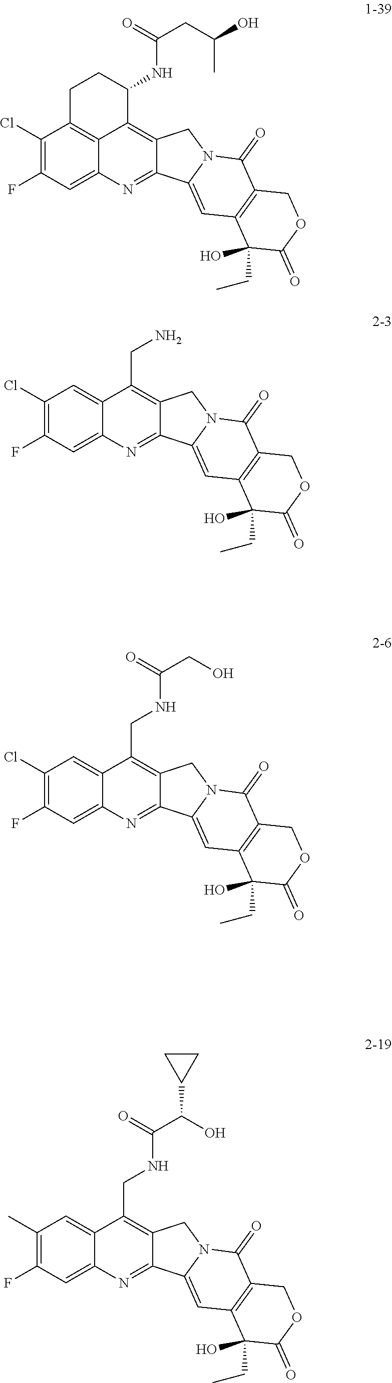 [SMILES: CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(F)c(C)cc1c2CNC(=O)[C@@H](O)C1CC1] 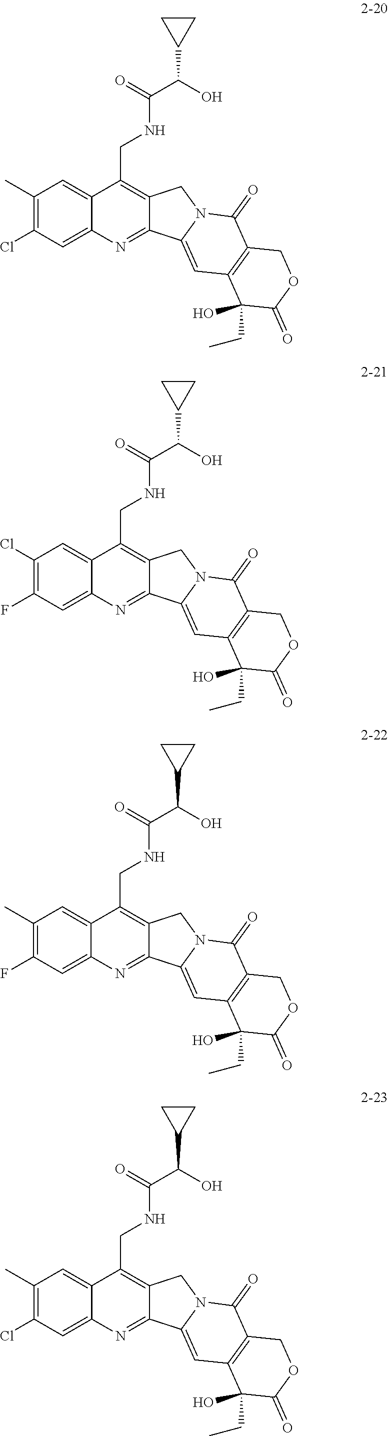 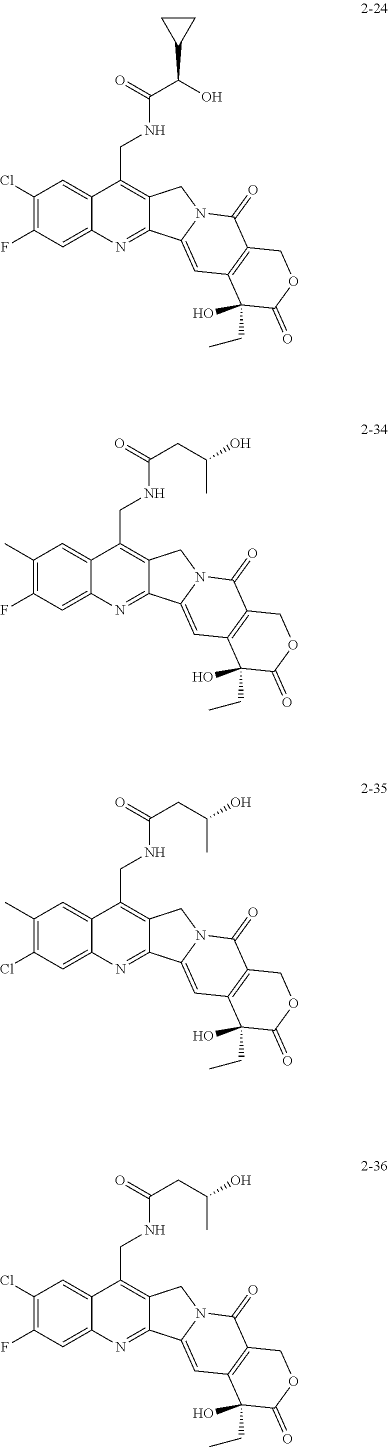 [SMILES: CC[C@@]1(O)C(=O)OCc2c1cc1n(c2=O)Cc2c-1nc1cc(F)c(Cl)cc1c2CNC(=O)[C@H](O)C1CC1 ; CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O] 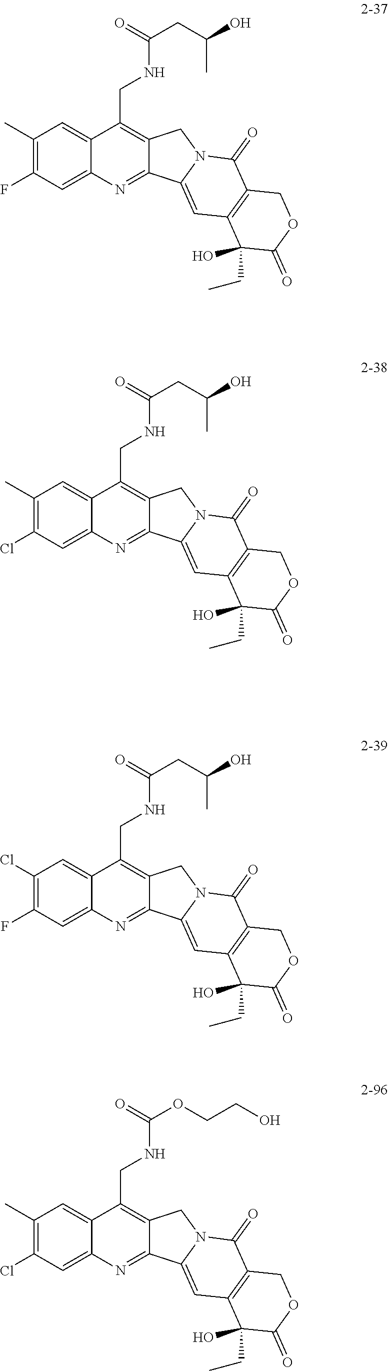 [SMILES: CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2CNC(=O)OCCO] 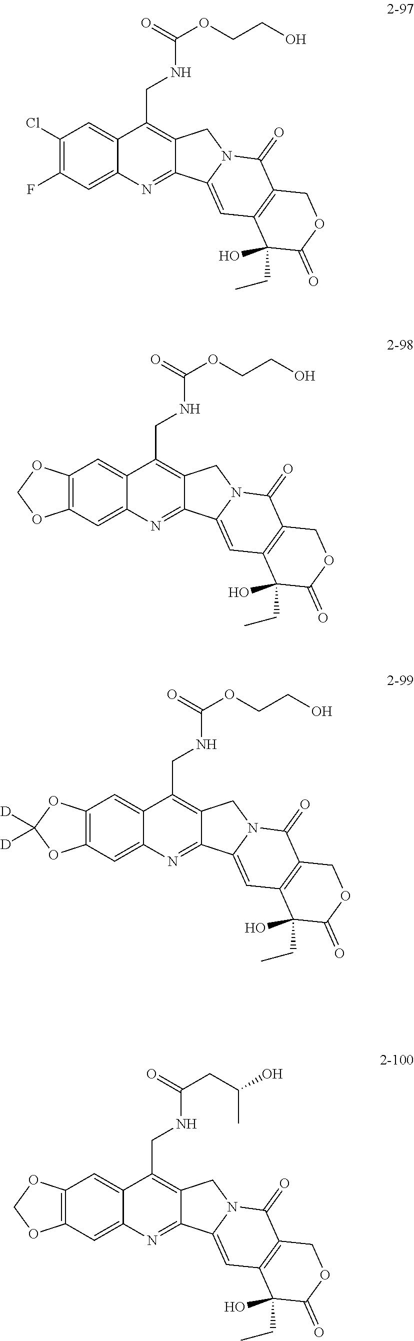 [SMILES: CC[C@@]1(O)C(=O)OCc2c1cc1n(c2=O)Cc2c-1nc1cc(F)c(Cl)cc1c2CNC(=O)OCCO ; CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC ; CC[C@@]1(O)C(=O)OCc2c1cc1n(c2=O)Cc2c-1nc1cc3c(cc1c2CNC(=O)C[C@@H](C)O)OCO3] 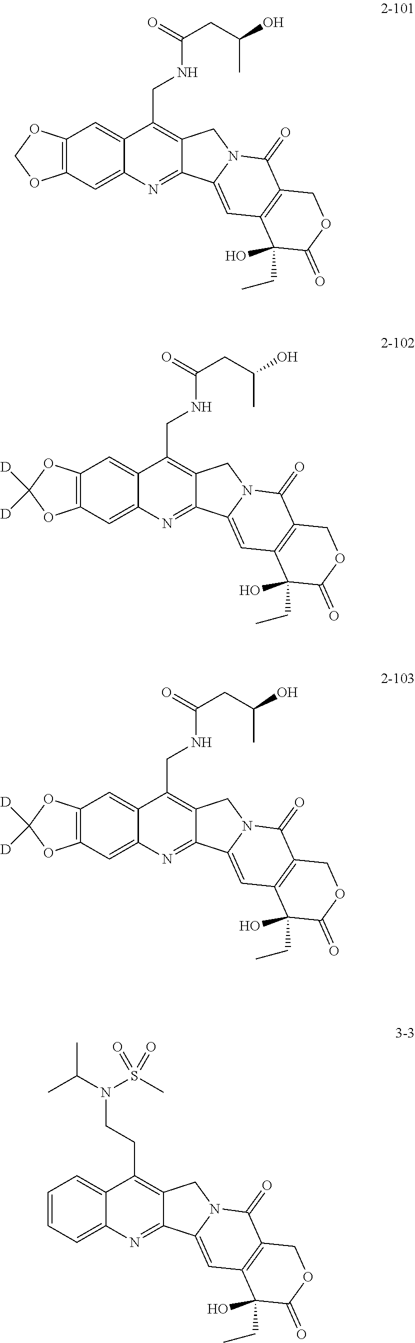 [SMILES: CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCN(C(C)C)S(C)(=O)=O] 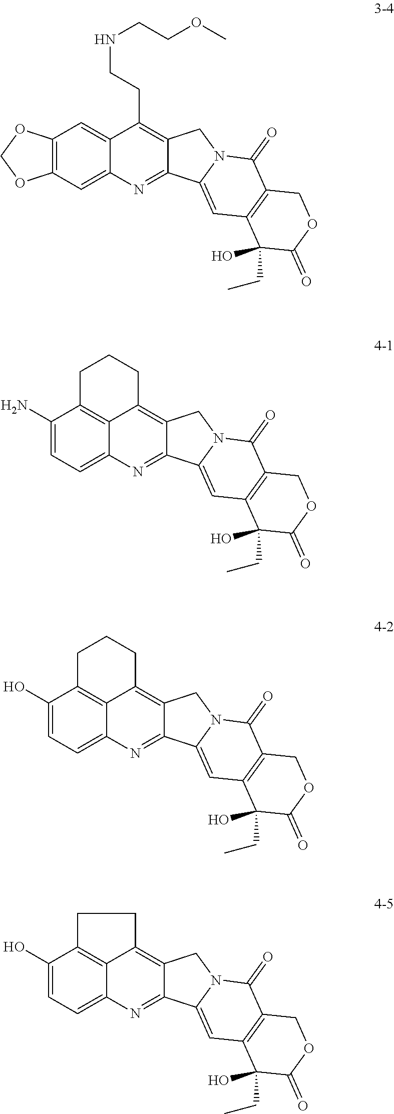 [SMILES: CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2CC3] 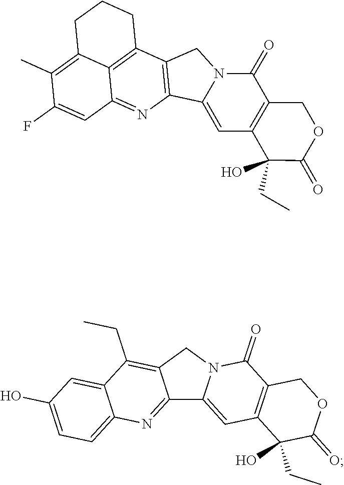 [SMILES: CC[C@@]1(O)C(=O)OCc2c1cc1n(c2=O)Cc2c-1nc1cc(F)c(C)c3c1c2CCC3 ; CCc1c2c(nc3ccc(O)cc13)-c1cc3c(c(=O)n1C2)COC(=O)[C@]3(O)CC]
preferably, the cytotoxic drug is selected from the group consisting of the following compounds or isotopically labeled compounds thereof: 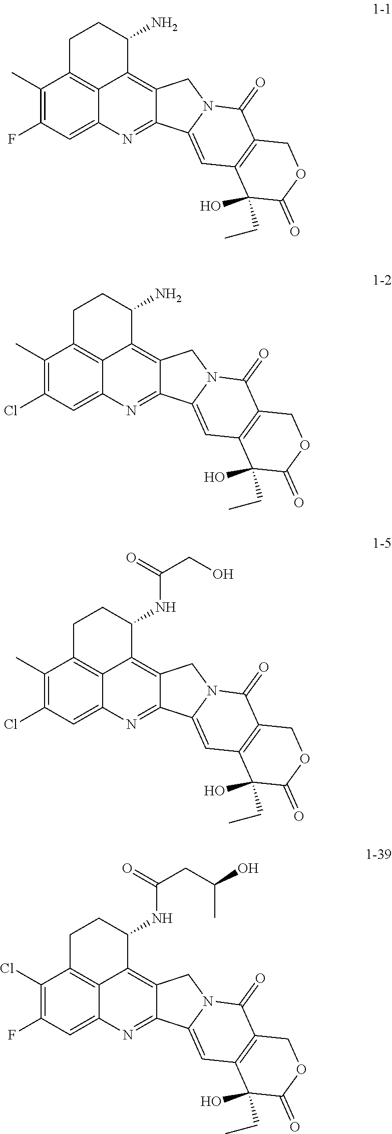 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3] 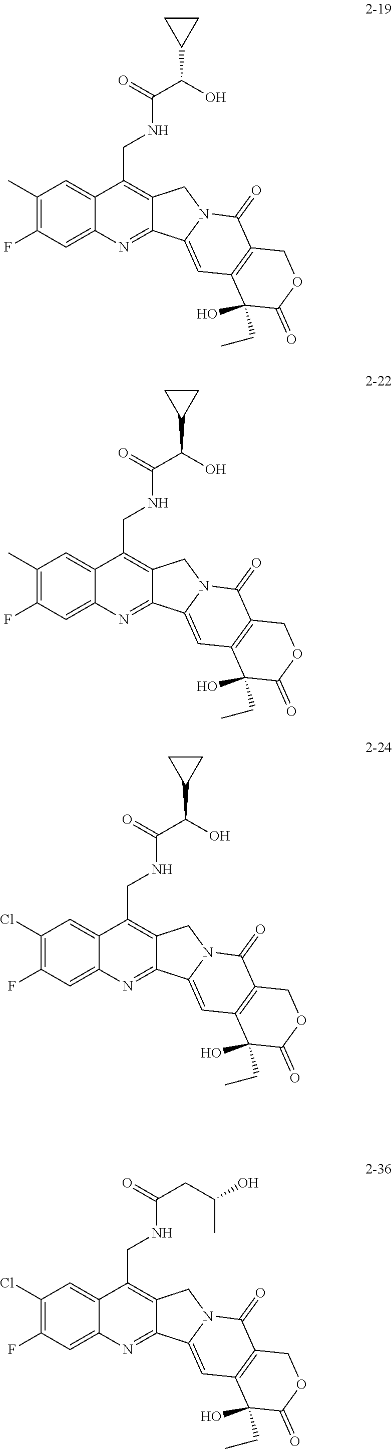 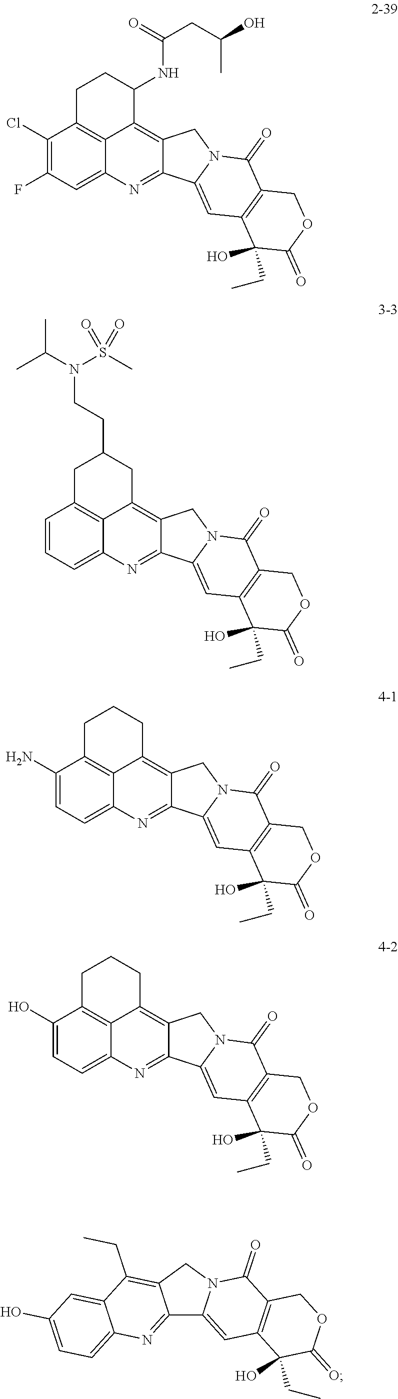 [SMILES: CC[C@@]1(O)C(=O)OCc2c1cc1n(c2=O)Cc2c-1nc1cc(F)c(Cl)c3c1c2C(NC(=O)C[C@H](C)O)CC3 ; CC[C@@]1(O)C(=O)OCc2c1cc1n(c2=O)Cc2c-1nc1cccc3c1c2CC(CCN(C(C)C)S(C)(=O)=O)C3 ; CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3 ; CCc1c2c(nc3ccc(O)cc13)-c1cc3c(c(=O)n1C2)COC(=O)[C@]3(O)CC]
preferably, the cytotoxic drug is selected from the group consisting of the following compounds or isotopically labeled compounds thereof: 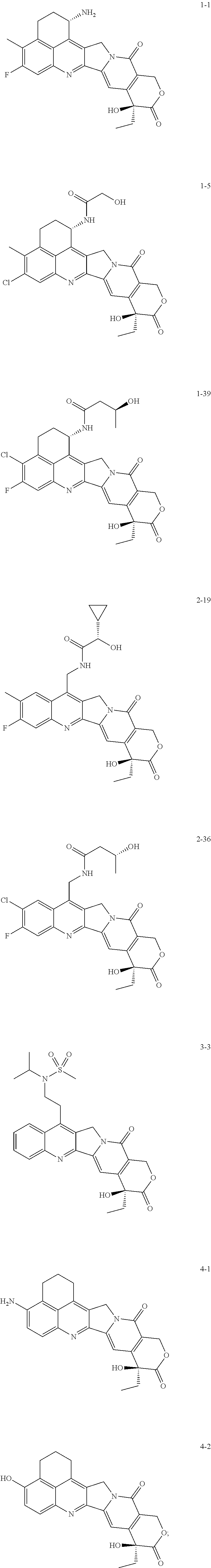
preferably, the cytotoxic drug is selected from the group consisting of the following compounds or isotopically labeled compounds thereof: 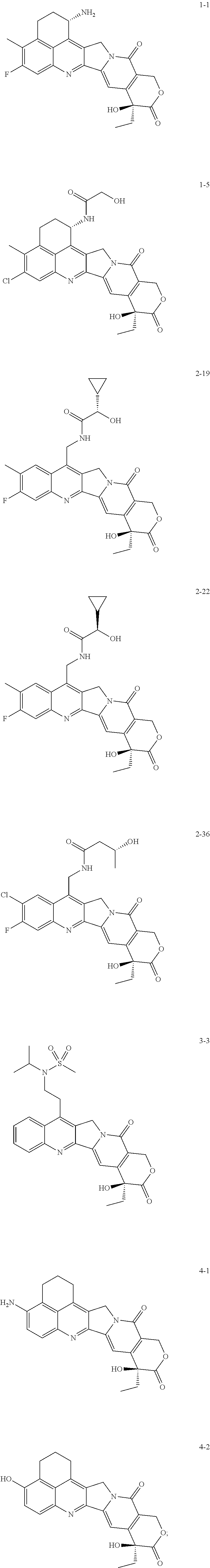
preferably, the cytotoxic drug is selected from the group consisting of the following compounds or isotopically labeled compounds thereof: 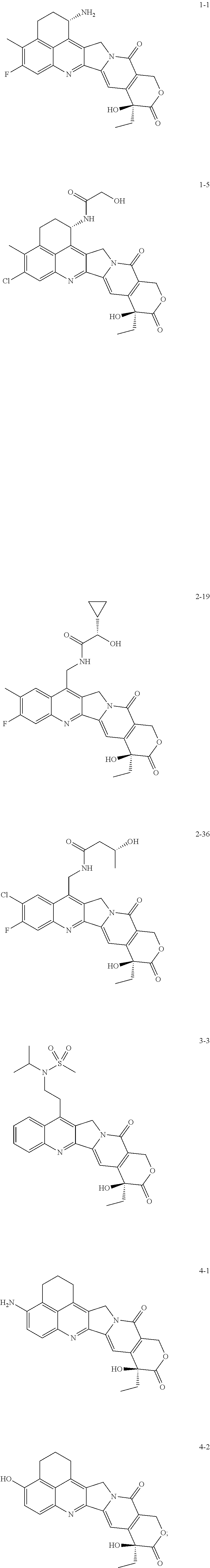
preferably, the cytotoxic drug is selected from the group consisting of the following compounds or isotopically labeled compounds thereof: 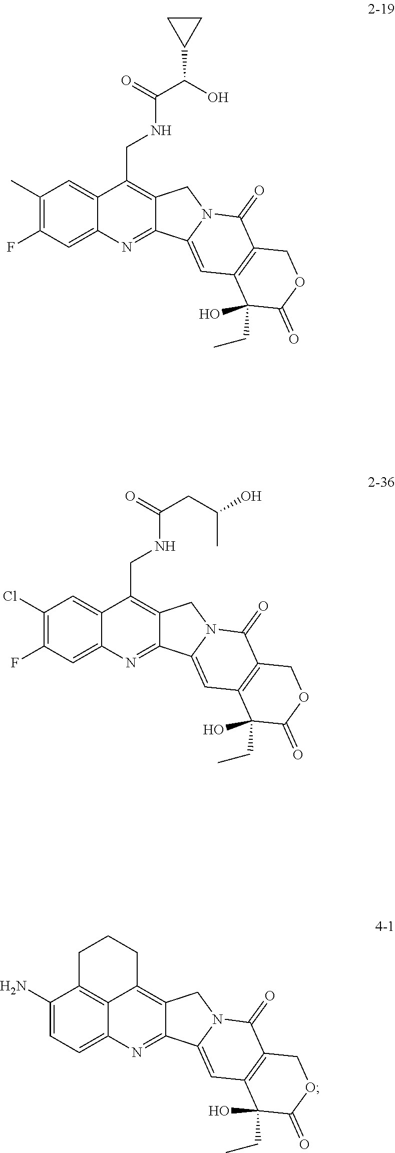 [SMILES: CC[C@@]1(O)C(=O)OCc2c1cc1n(c2=O)Cc2c-1nc1cc(F)c(C)cc1c2CNC(=O)[C@@H](O)C1CC1 ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1ccc(N)c3c1c2CCC3]
preferably, the cytotoxic drug is connected through —OH, primary amino group, secondary amine group or tertiary amine group thereon to E in the compound;
preferably, the D is selected from the group consisting of:

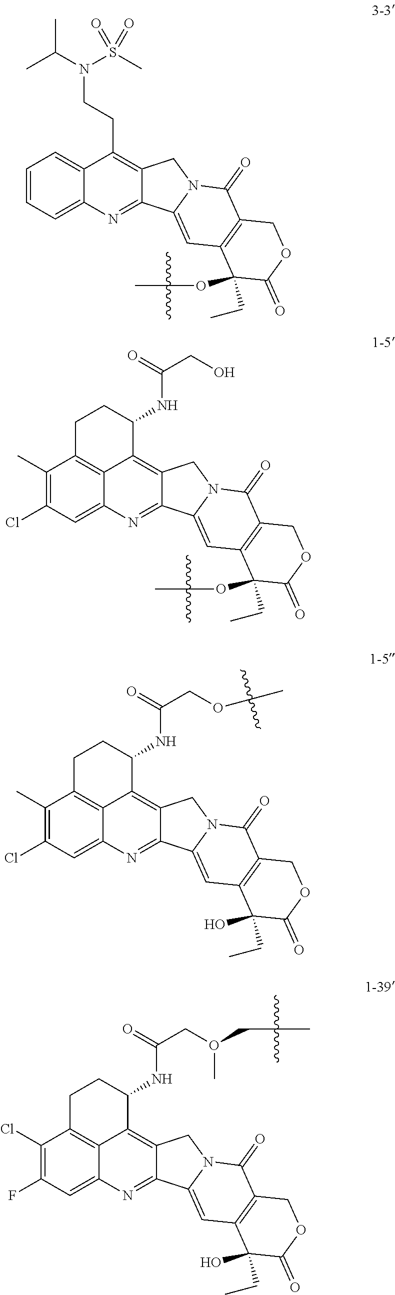

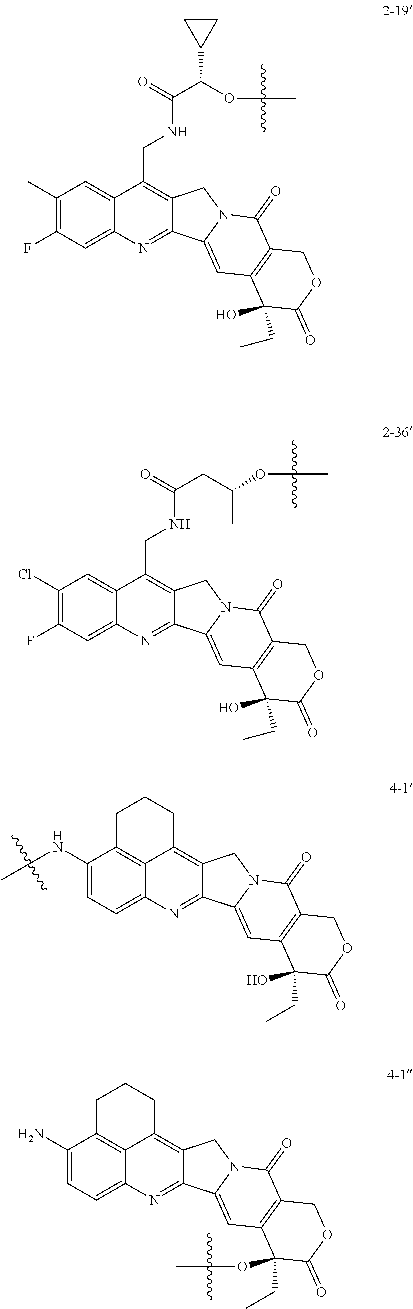 [SMILES: *O[C@H](C(=O)NCc1c2c(nc3cc(F)c(C)cc13)-c1cc3c(c(=O)n1C2)COC(=O)[C@]3(O)CC)C1CC1 ; *O[C@H](C)CC(=O)NCc1c2c(nc3cc(F)c(Cl)cc13)-c1cc3c(c(=O)n1C2)COC(=O)[C@]3(O)CC ; *Nc1ccc2nc3c(c4c2c1CCC4)Cn1c-3cc2c(c1=O)COC(=O)[C@]2(O)CC ; *O[C@]1(CC)C(=O)OCc2c1cc1n(c2=O)Cc2c-1nc1ccc(N)c3c1c2CCC3]

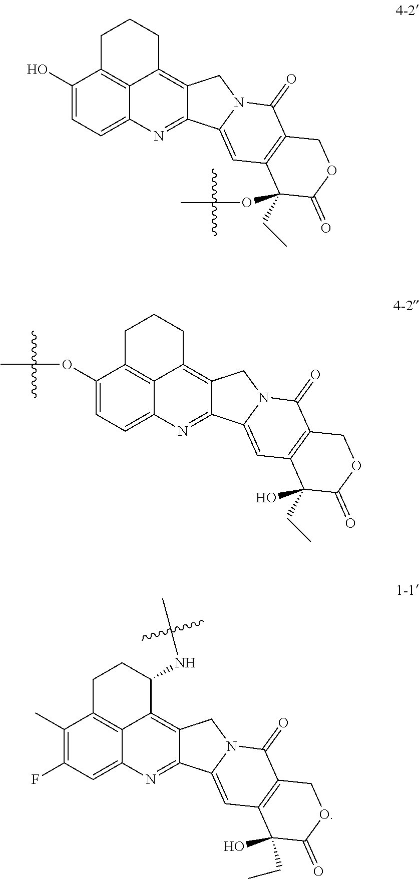 [SMILES: *O[C@]1(CC)C(=O)OCc2c1cc1n(c2=O)Cc2c-1nc1ccc(O)c3c1c2CCC3 ; *Oc1ccc2nc3c(c4c2c1CCC4)Cn1c-3cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC]

7. The compound or pharmaceutically acceptable salt thereof according to claim 1, wherein its structure is as follows: 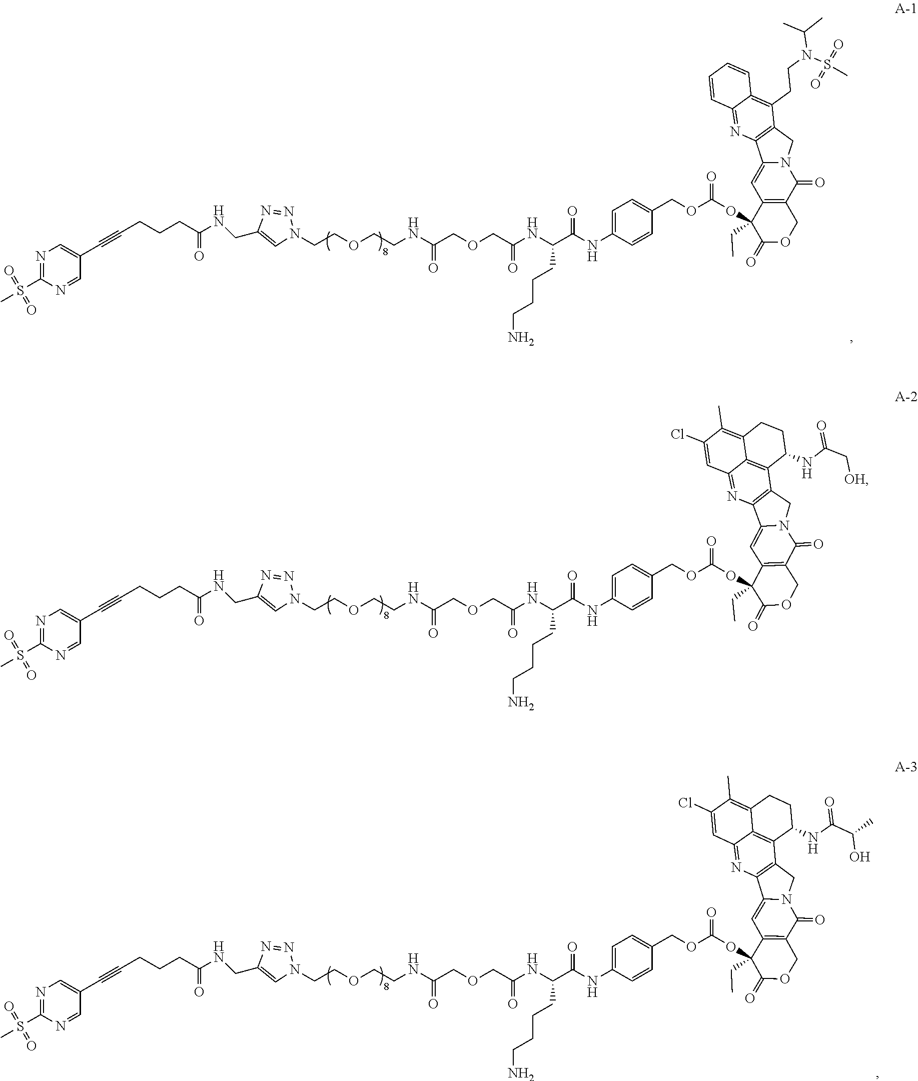 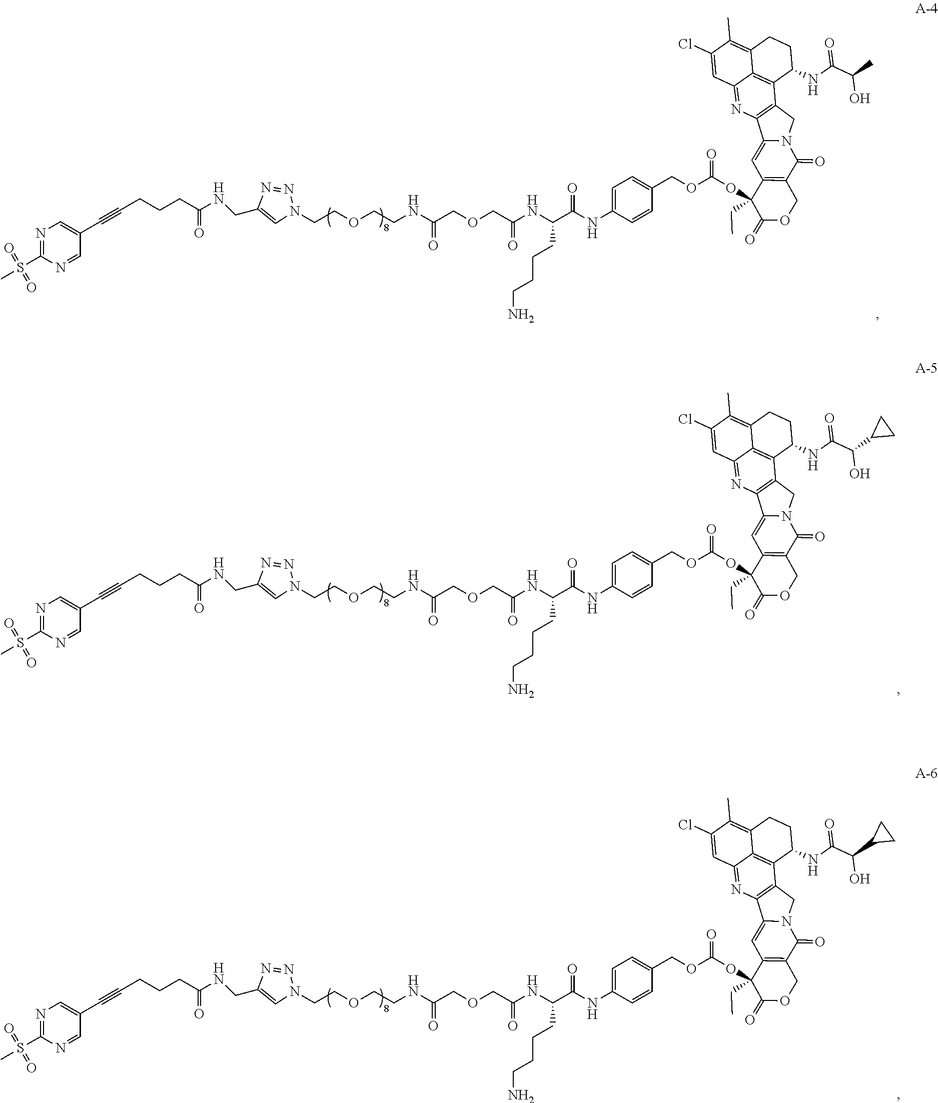 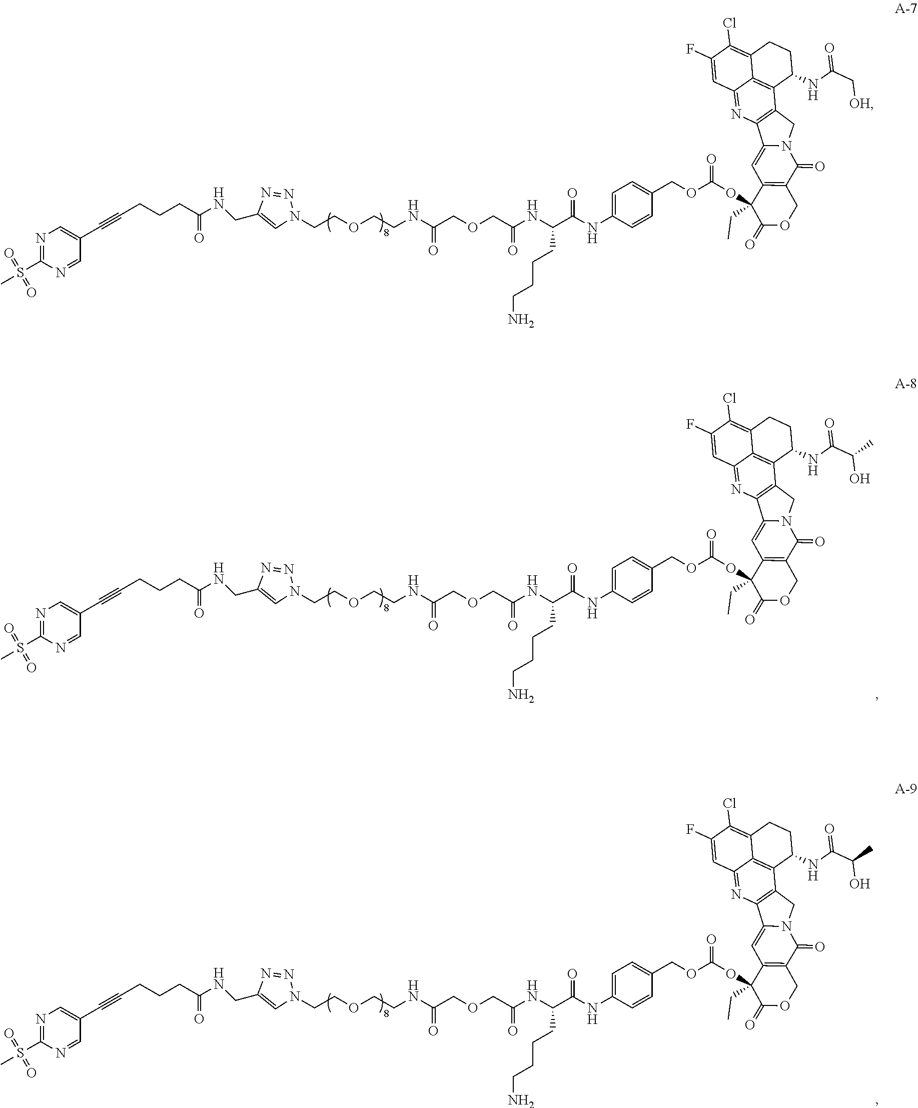 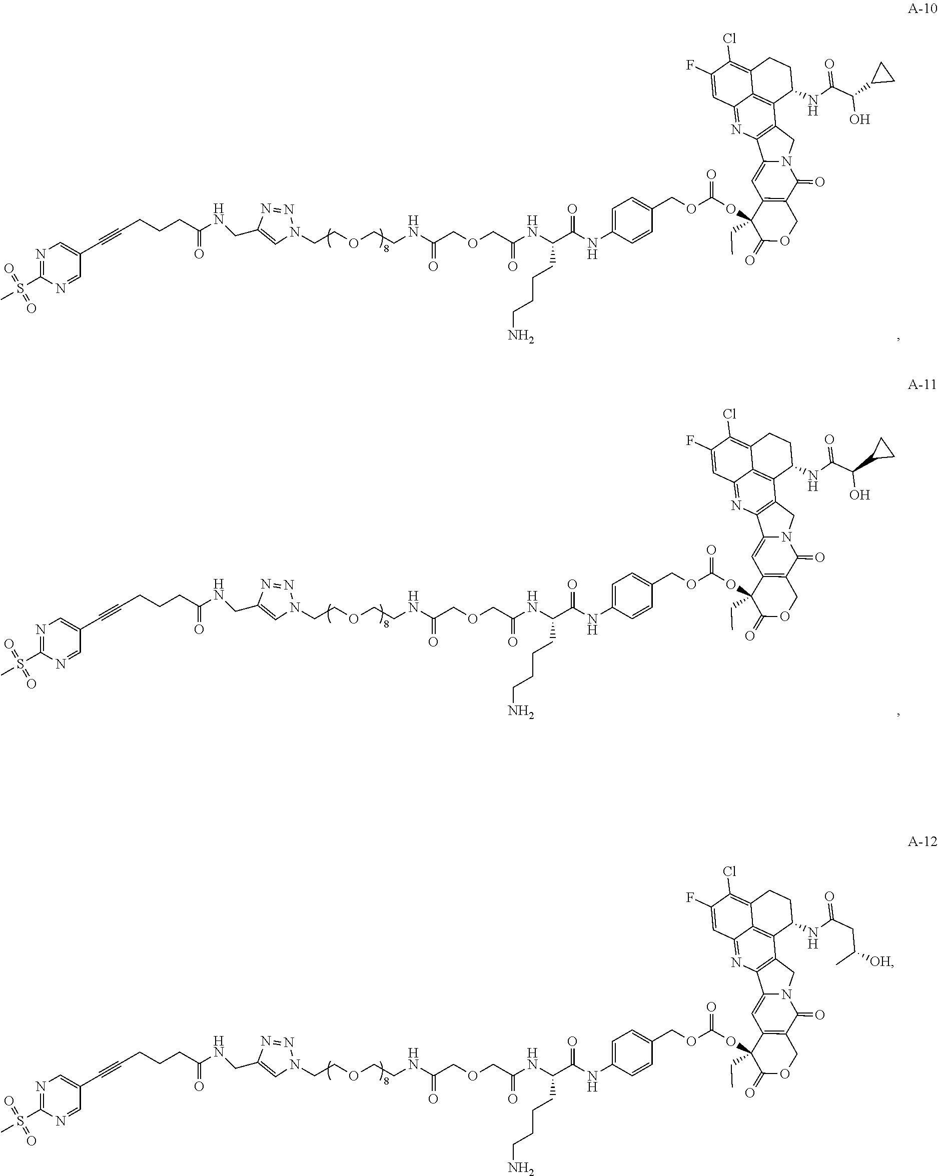 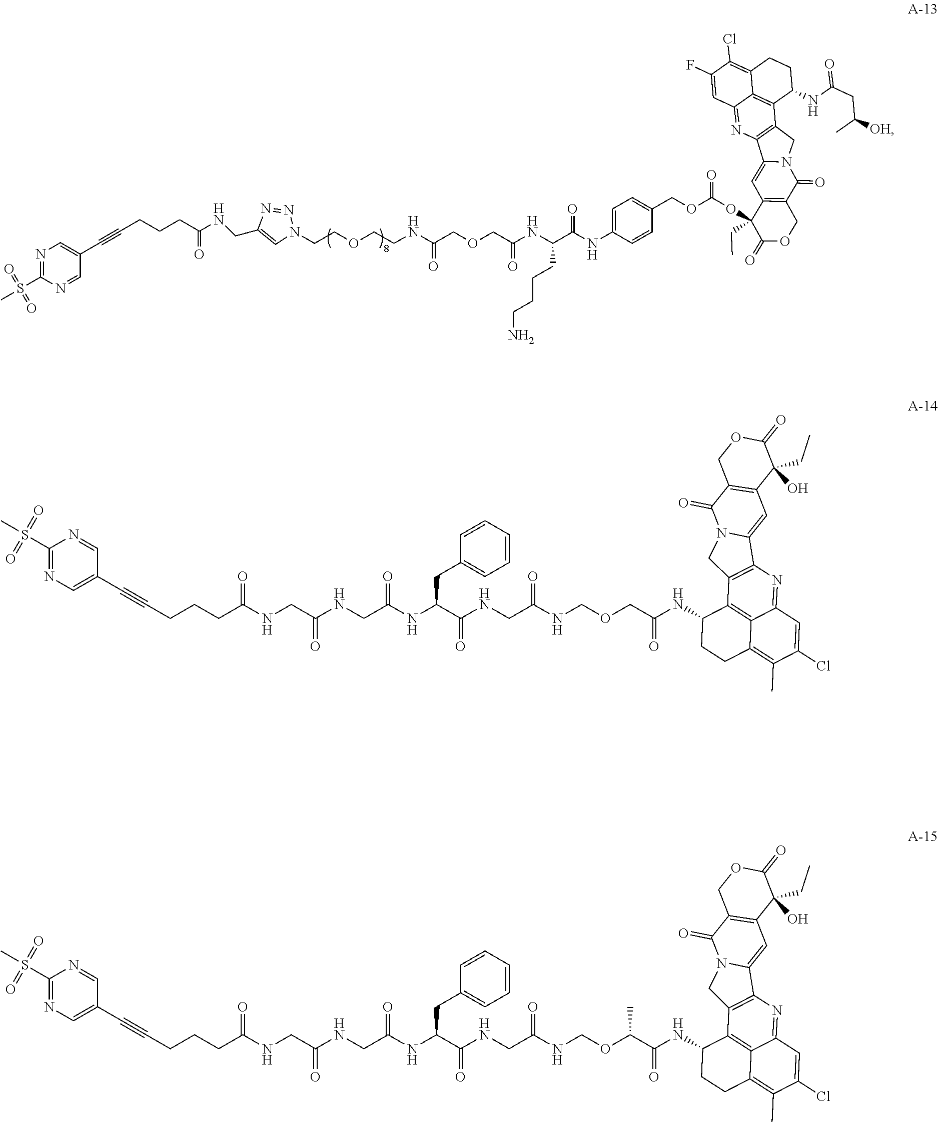 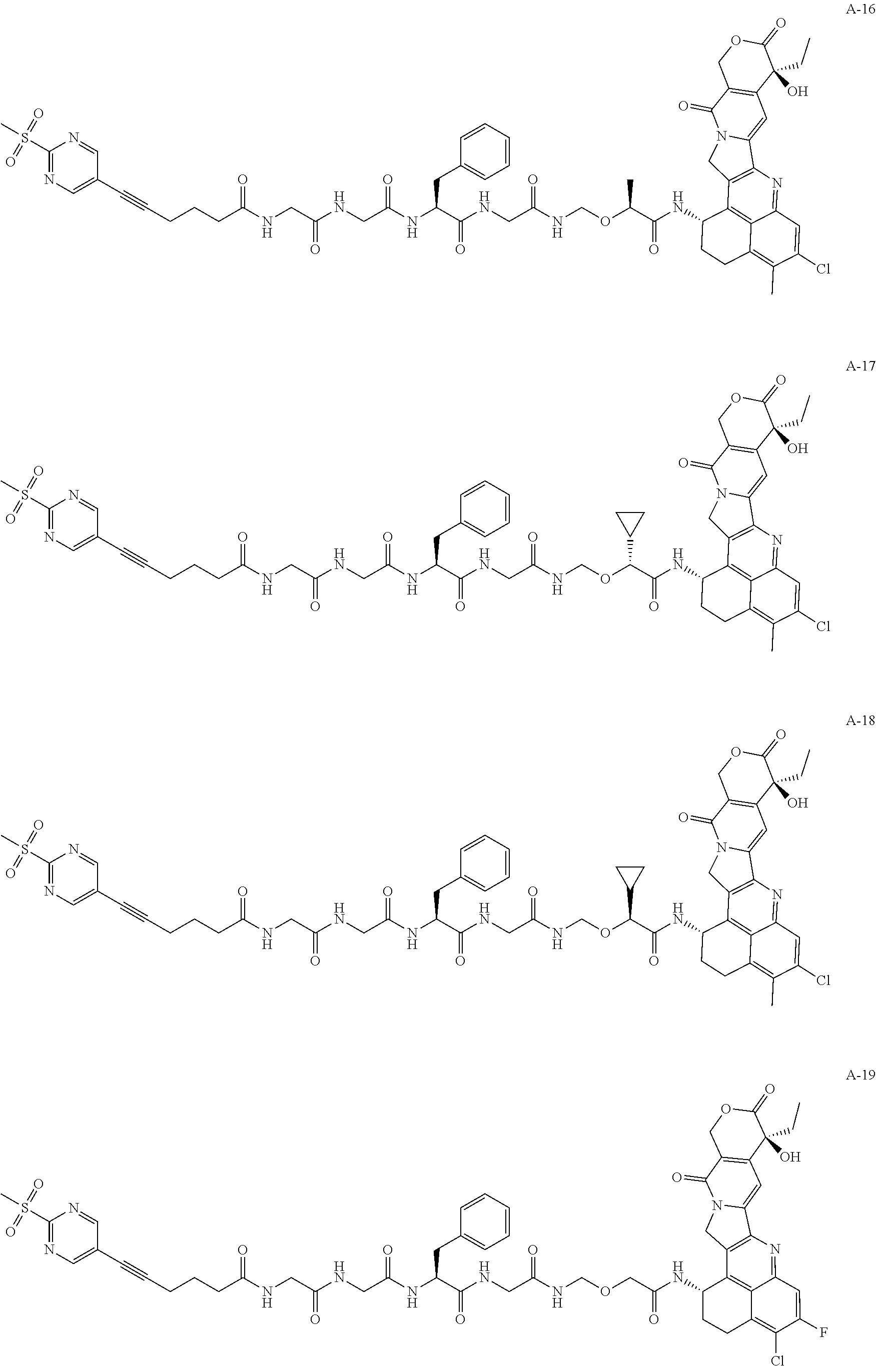 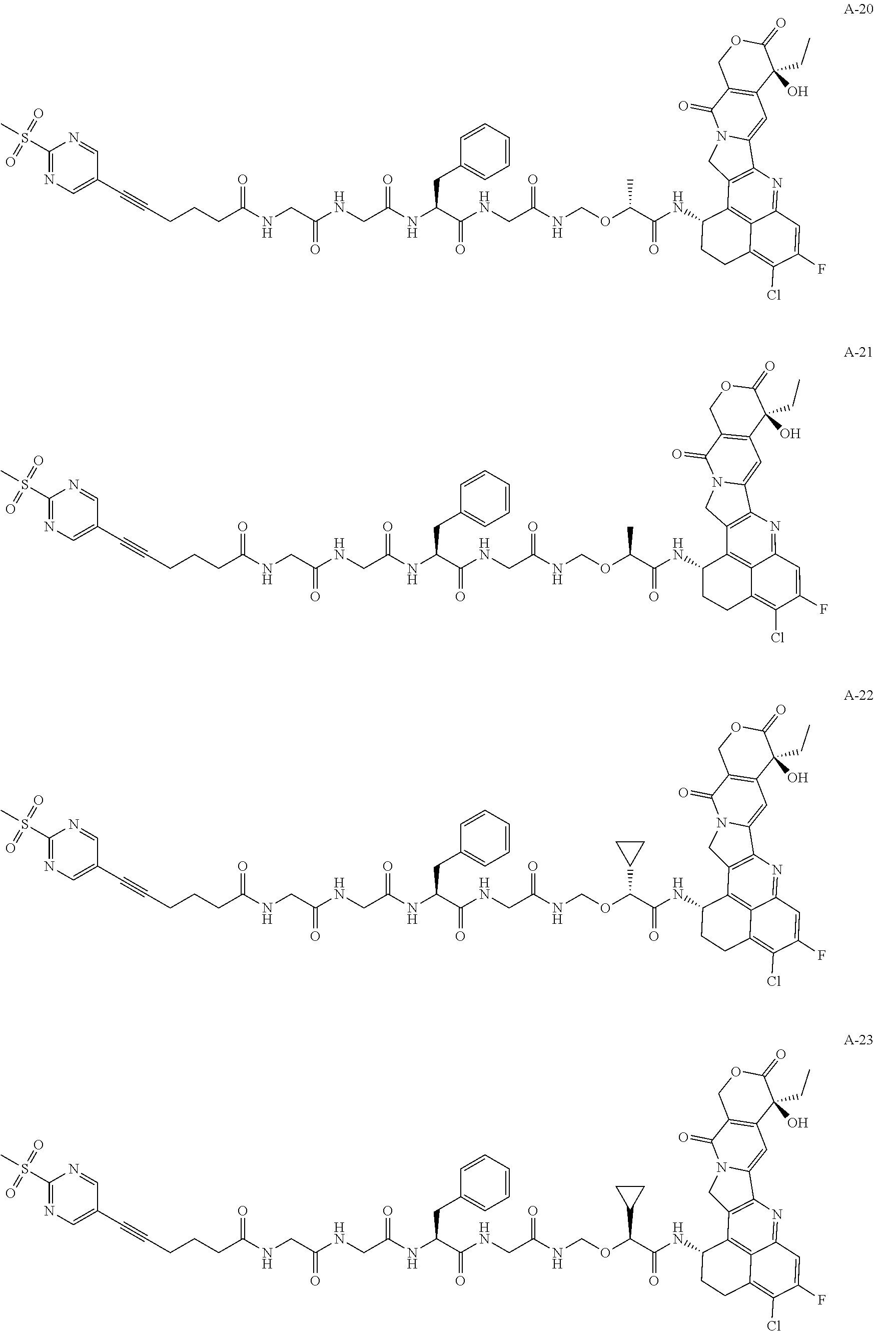 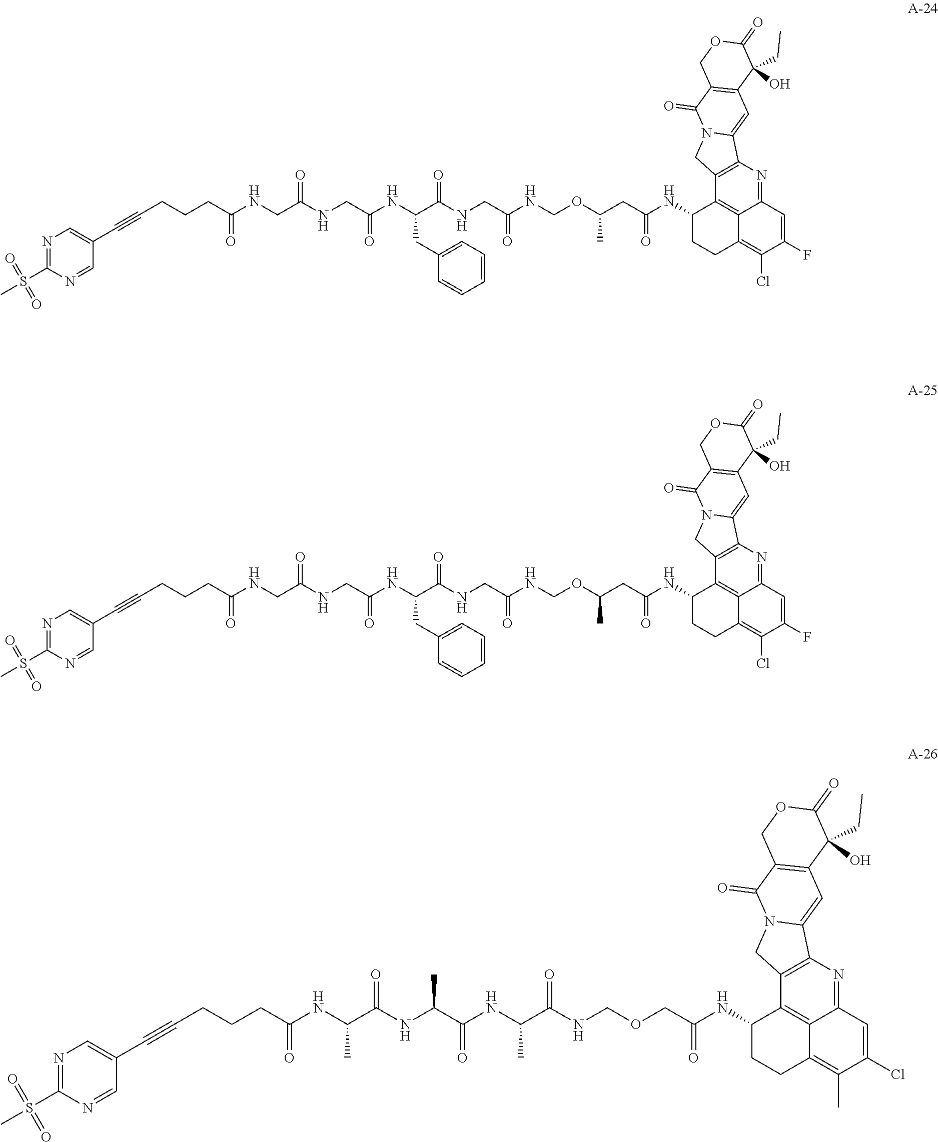 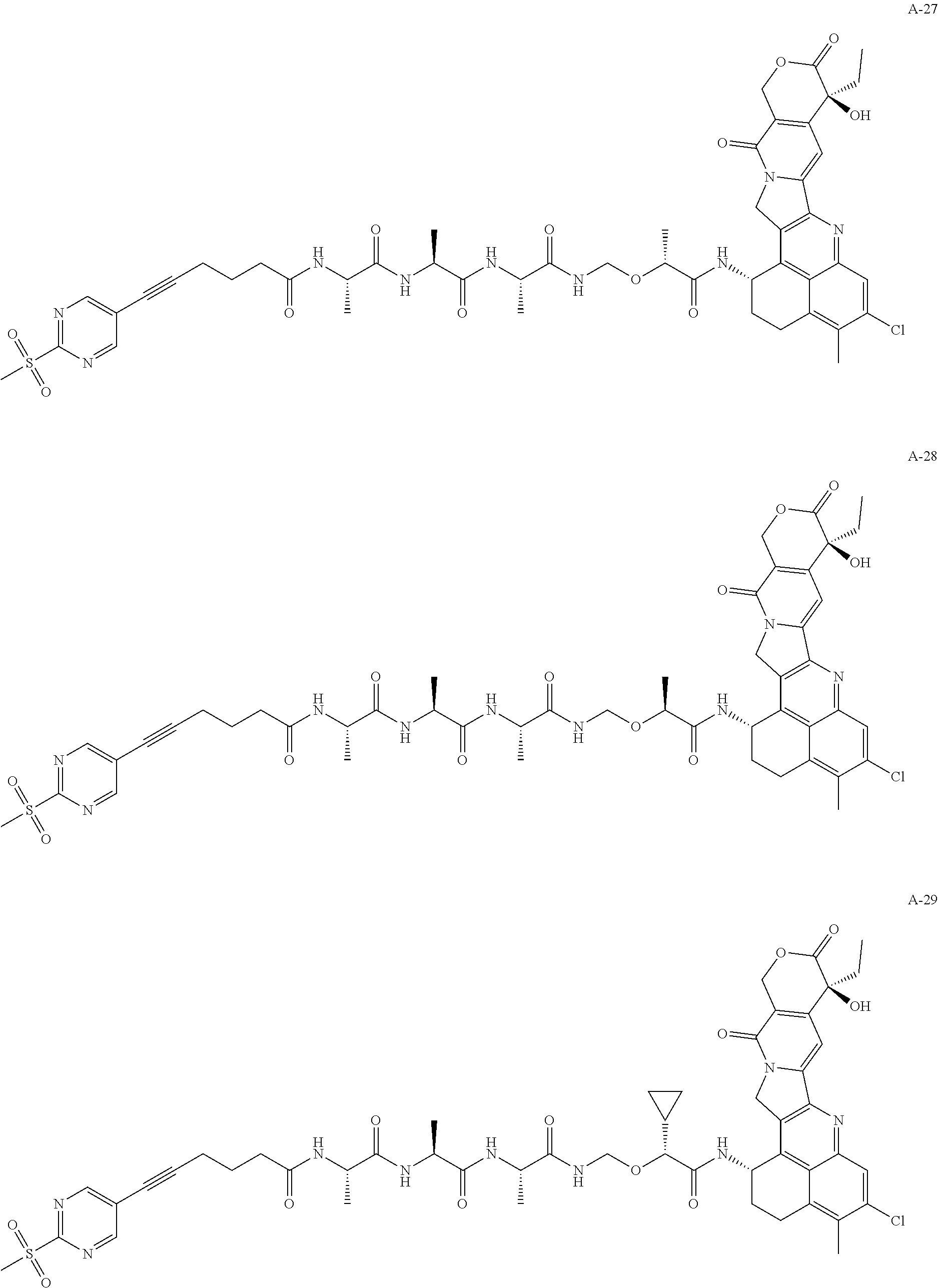 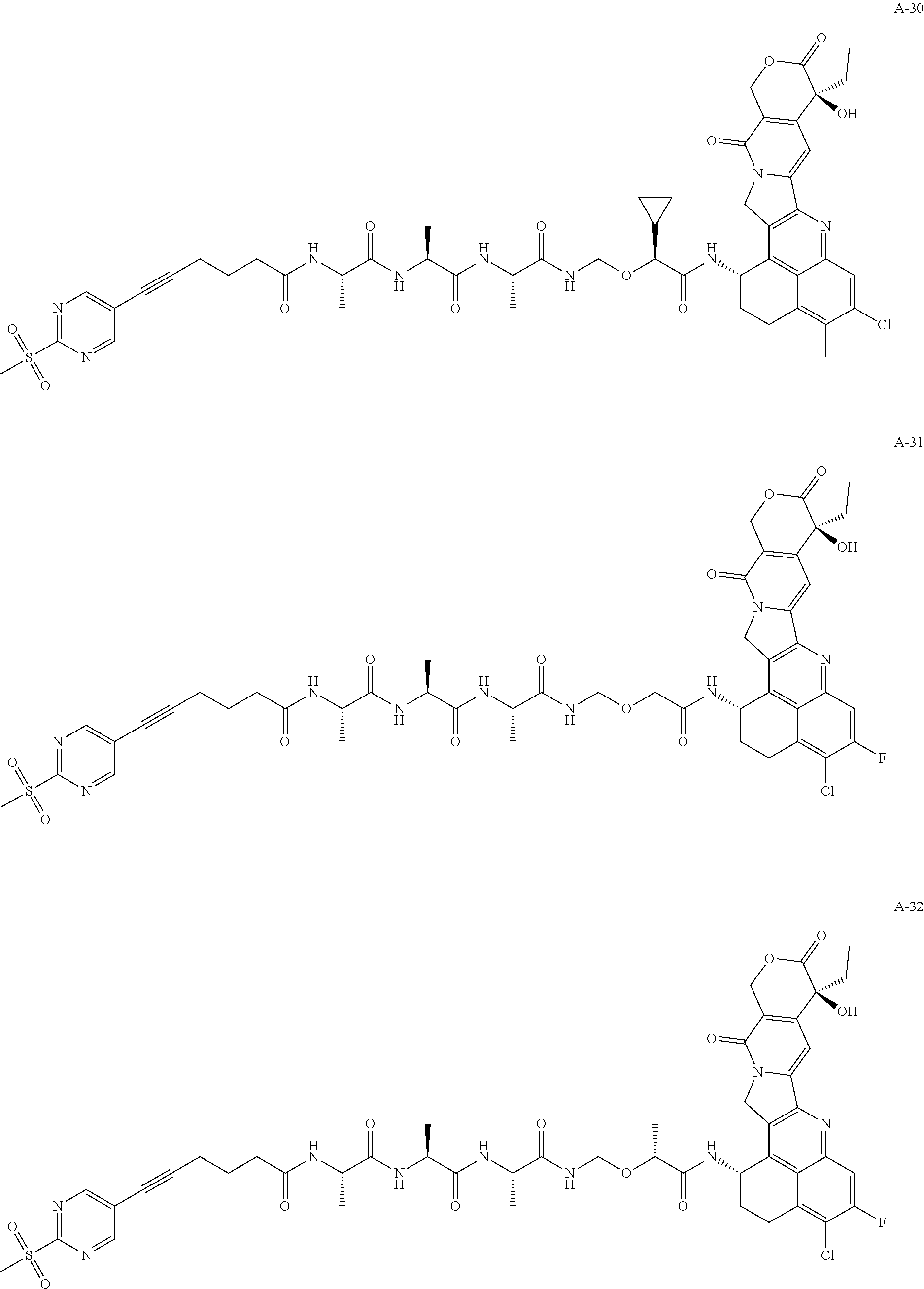 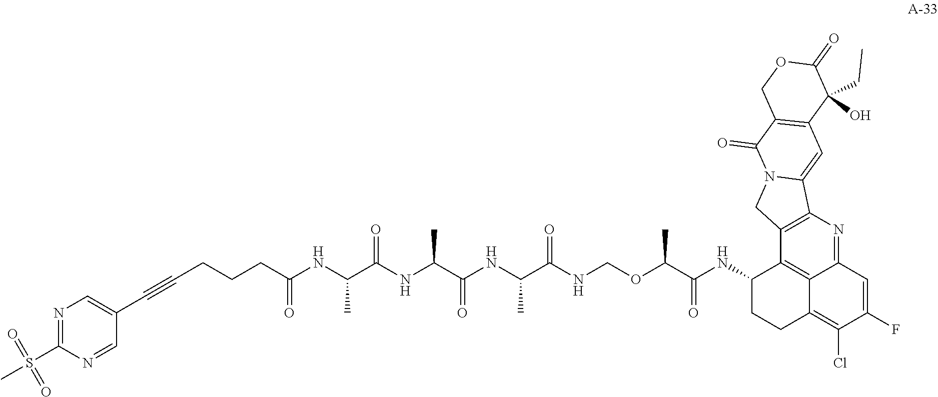 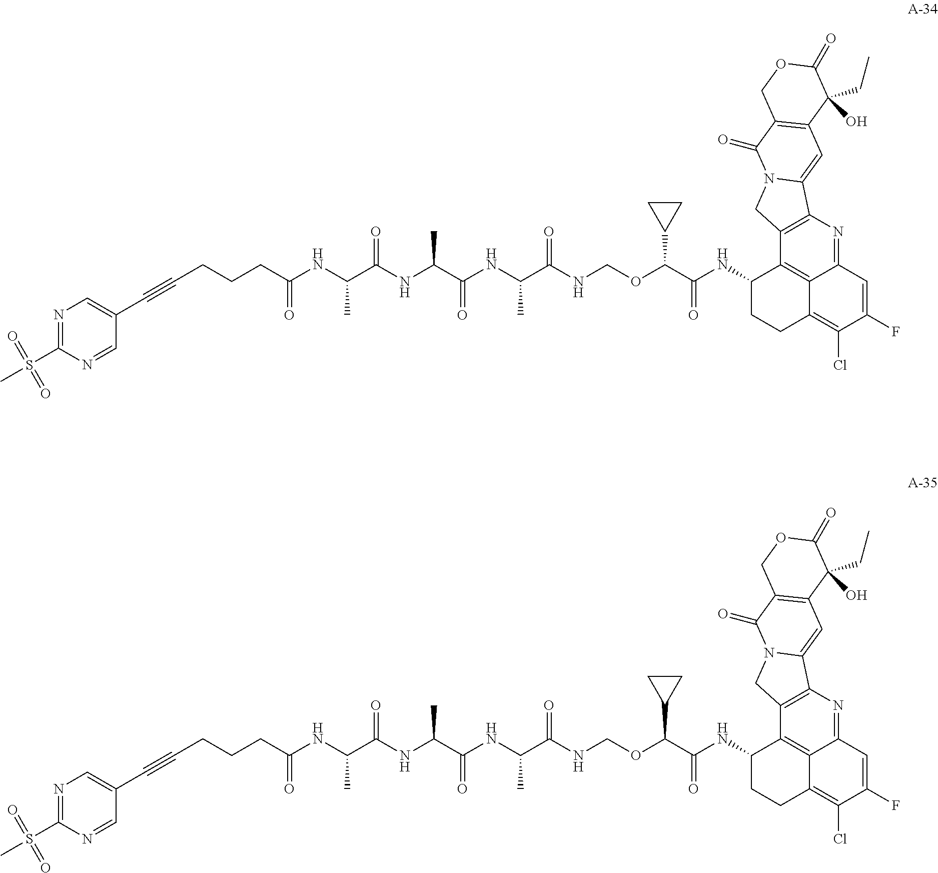 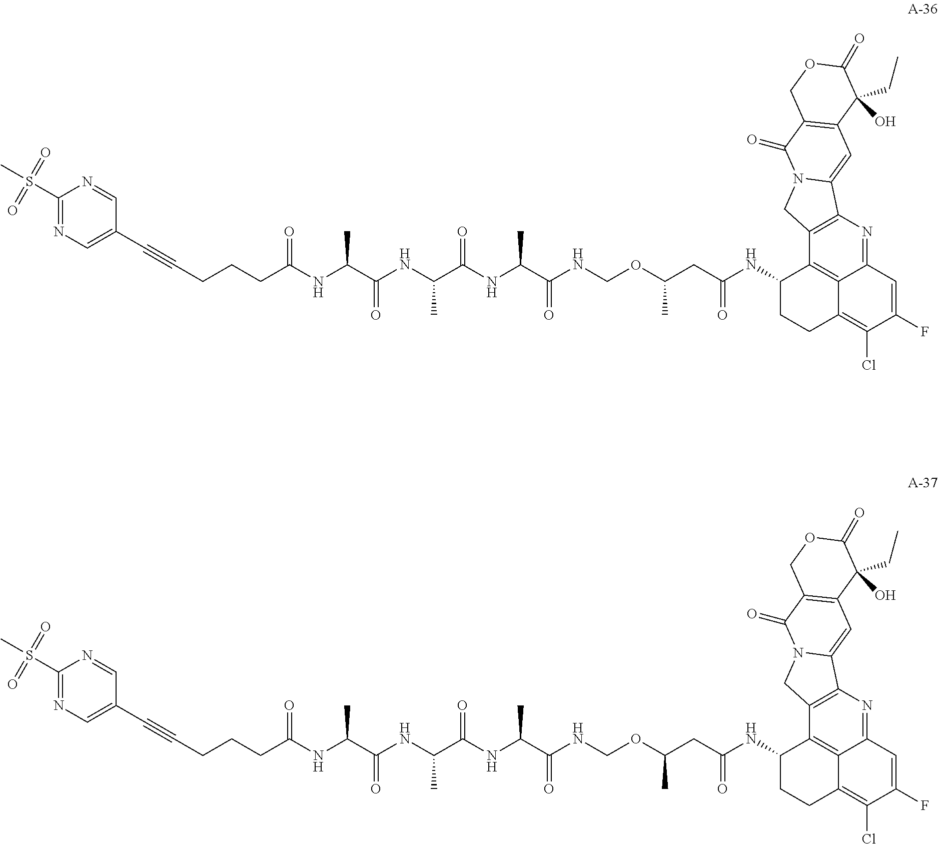 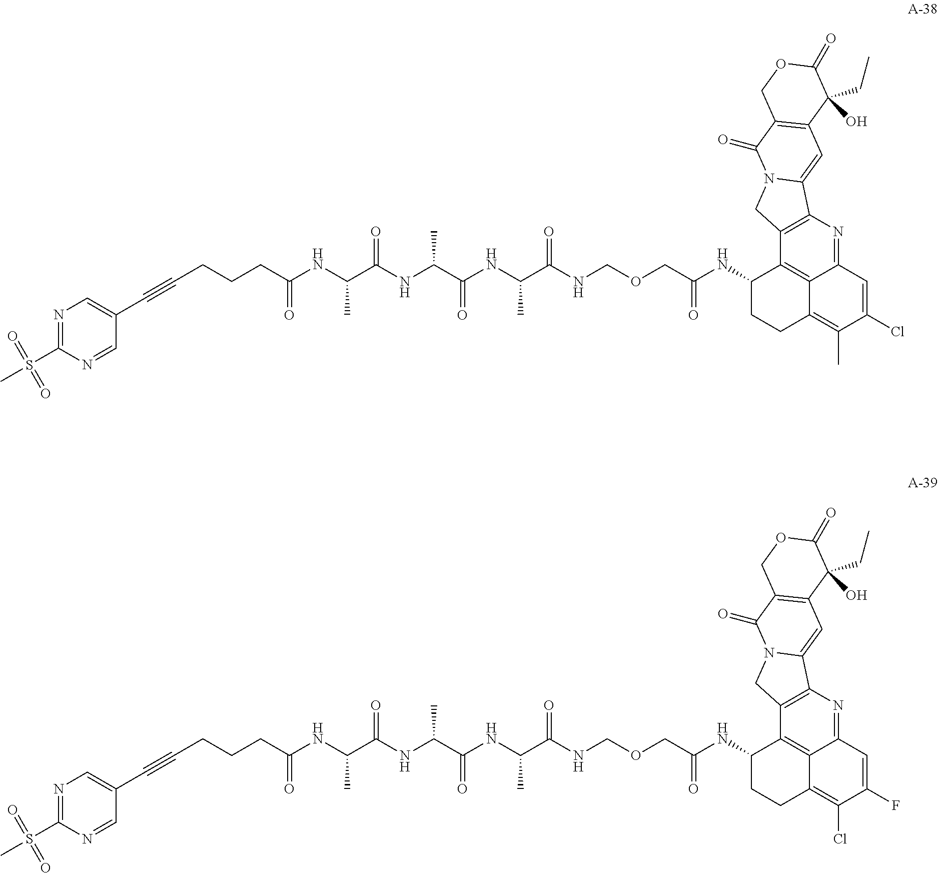 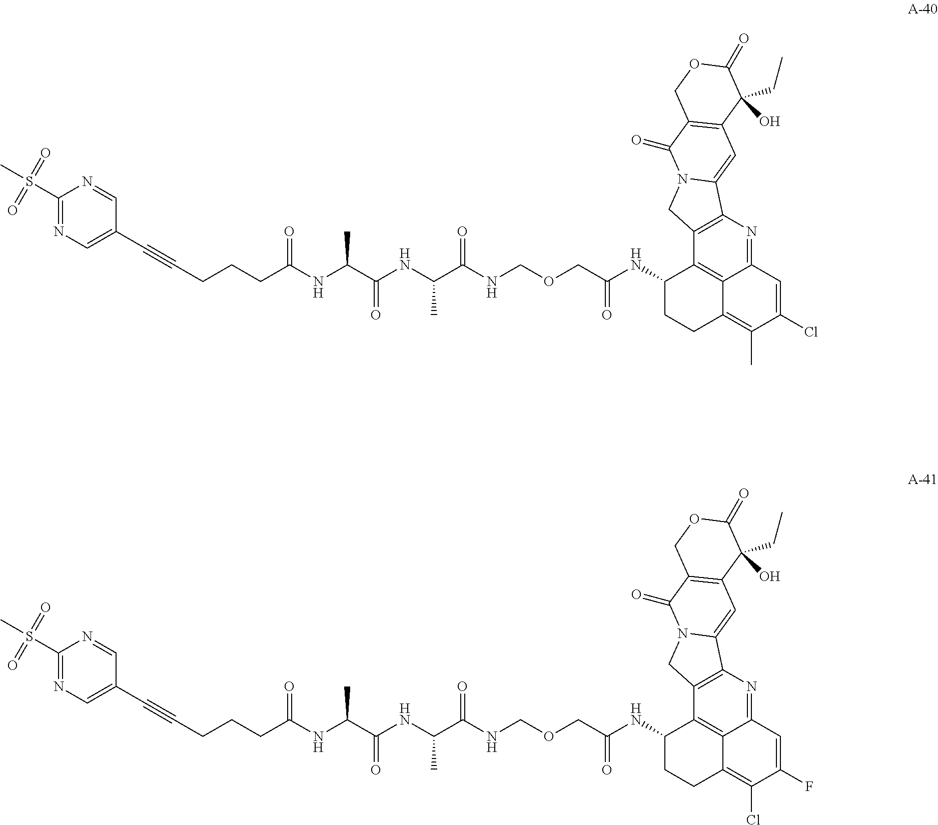 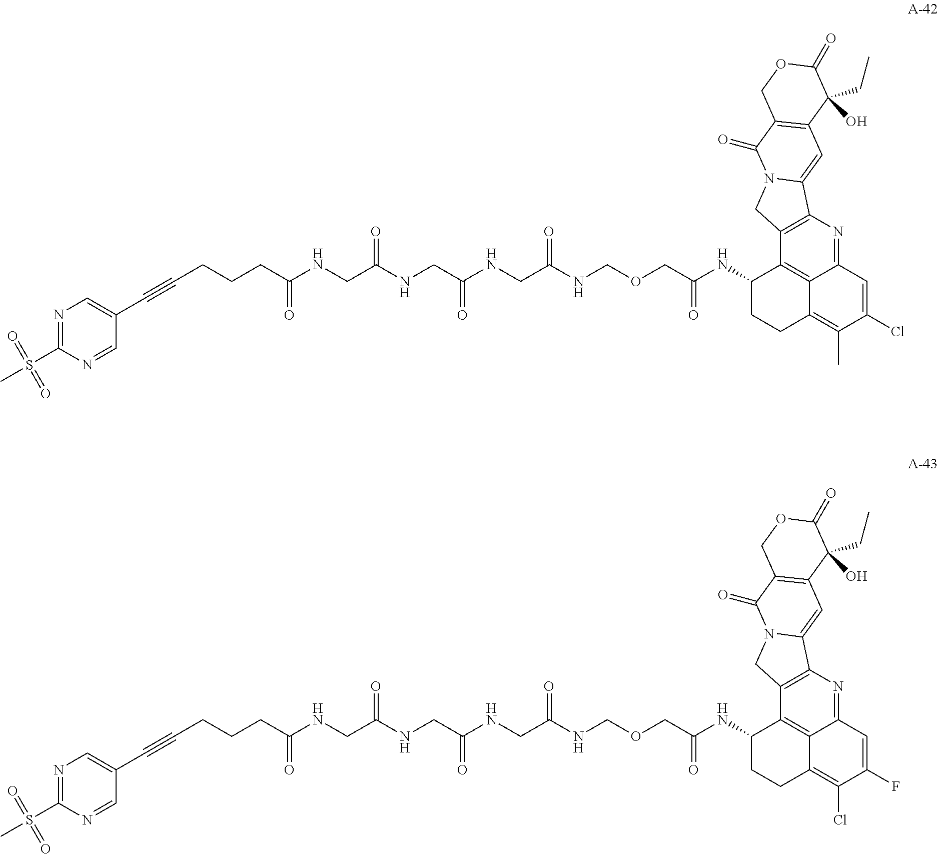 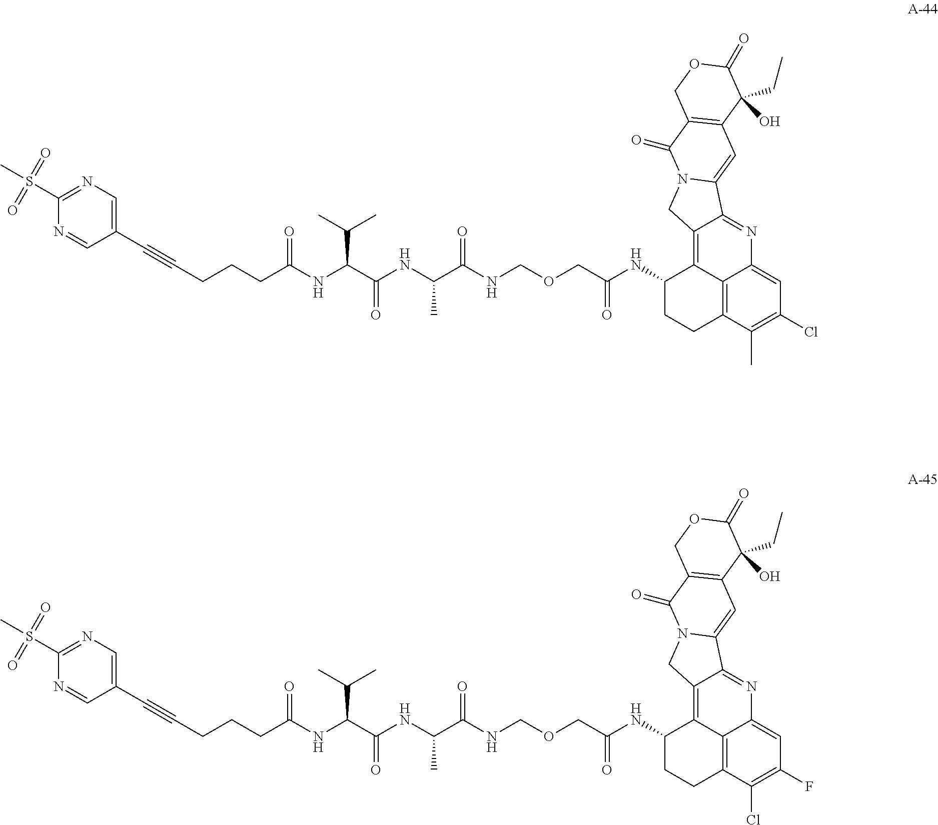 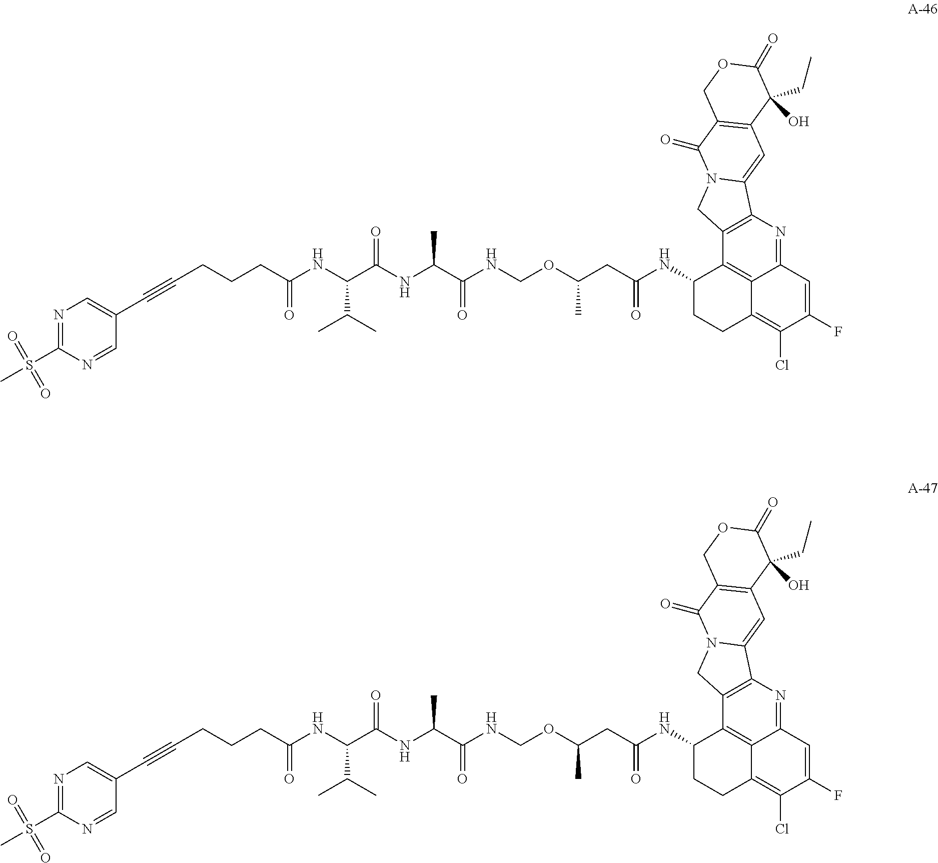 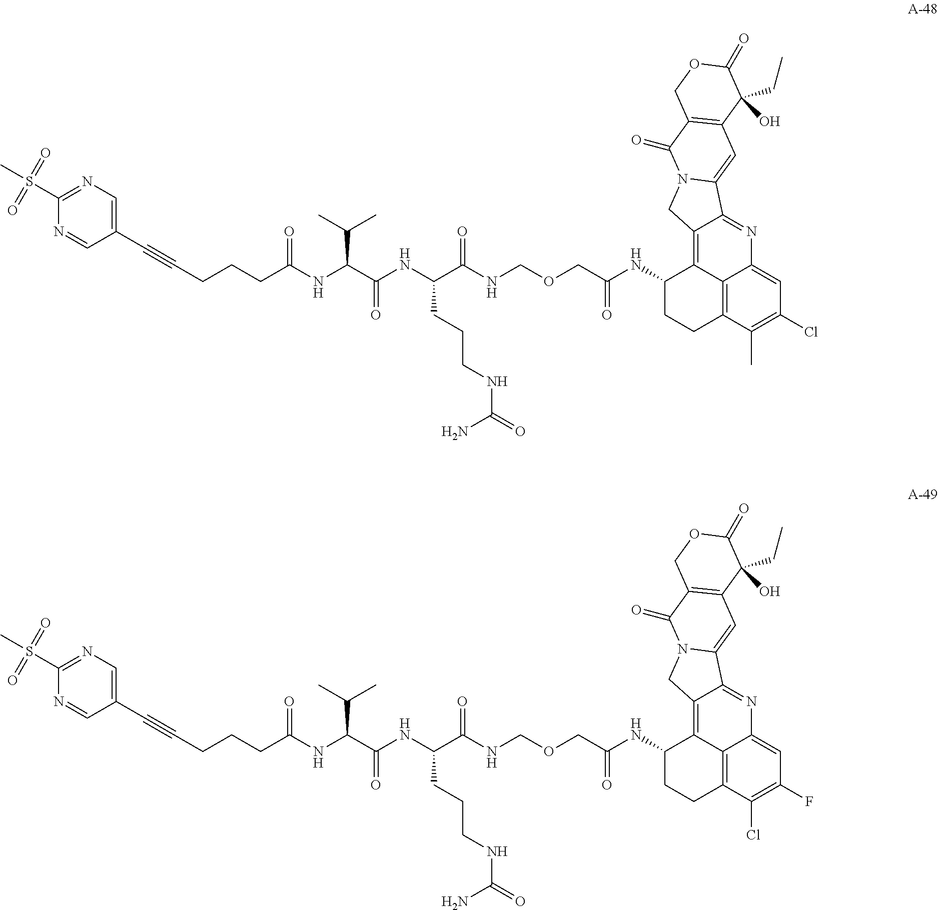 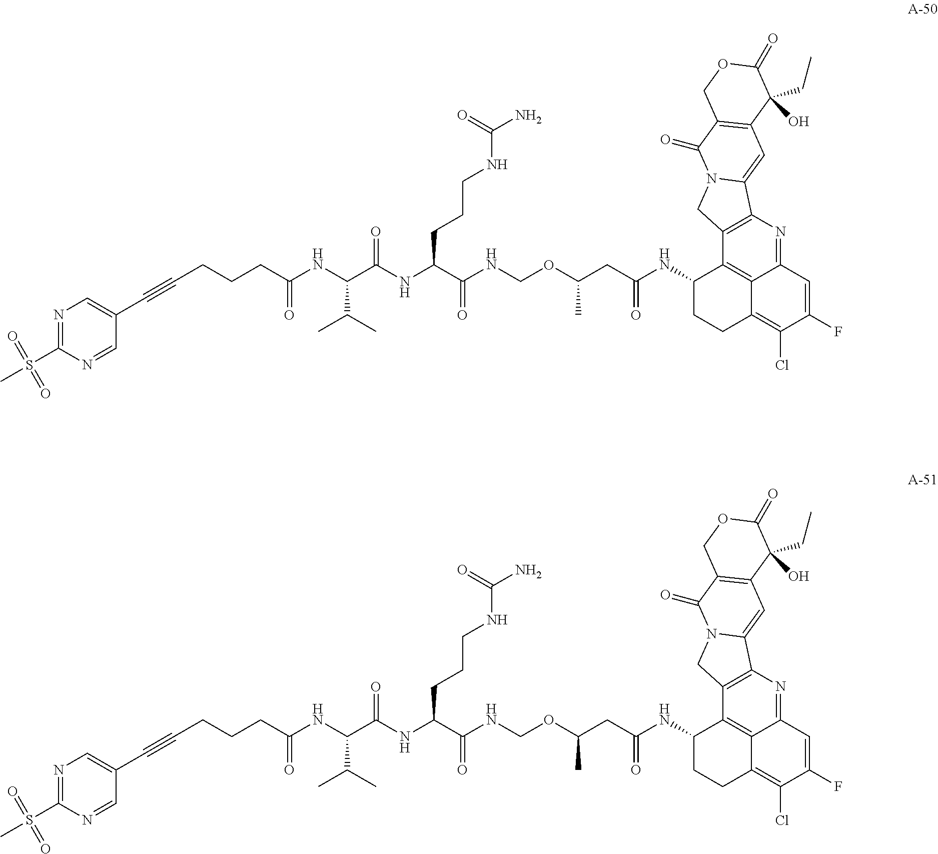 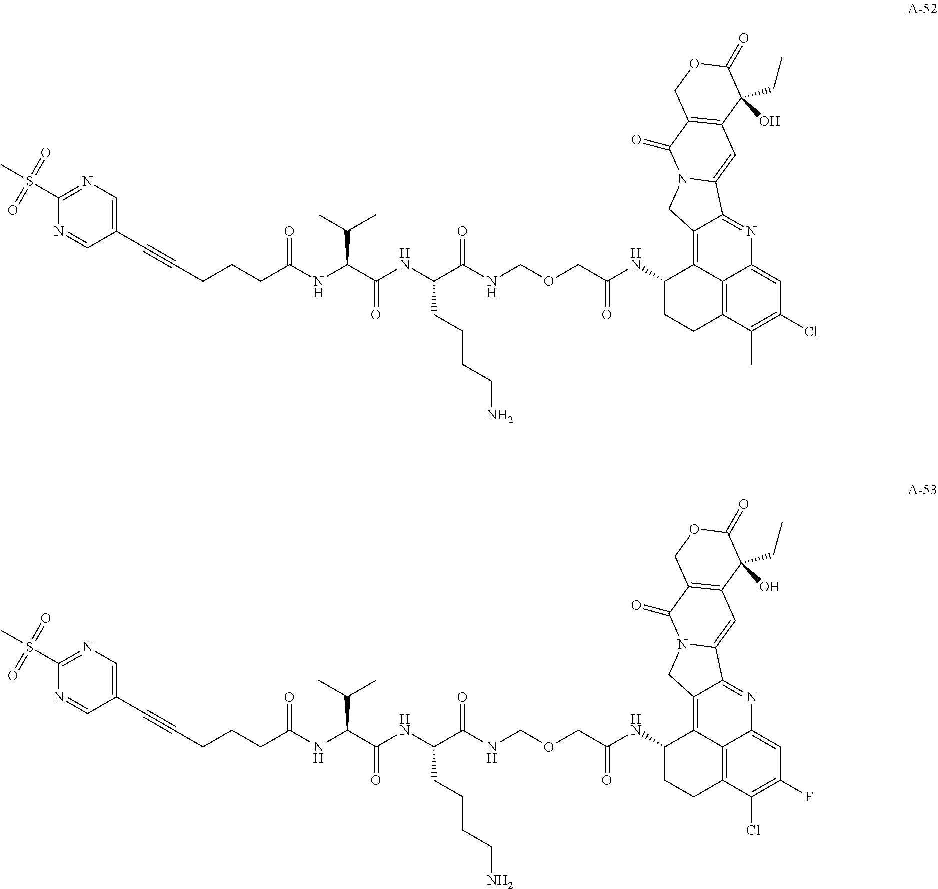 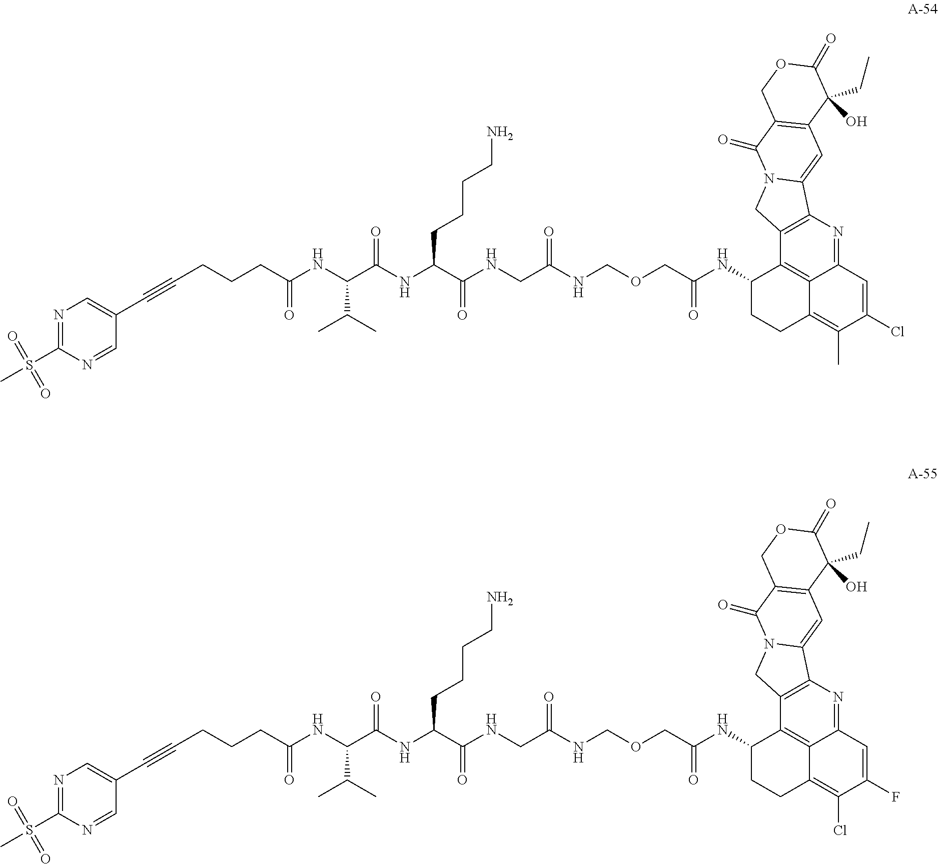 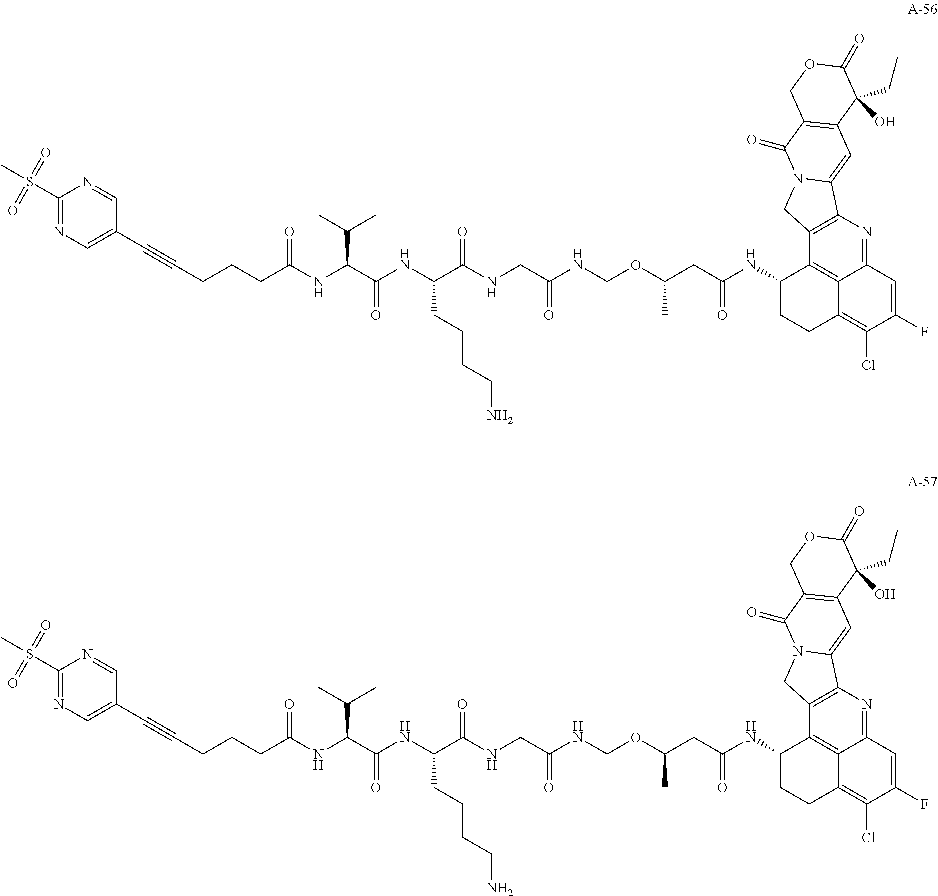 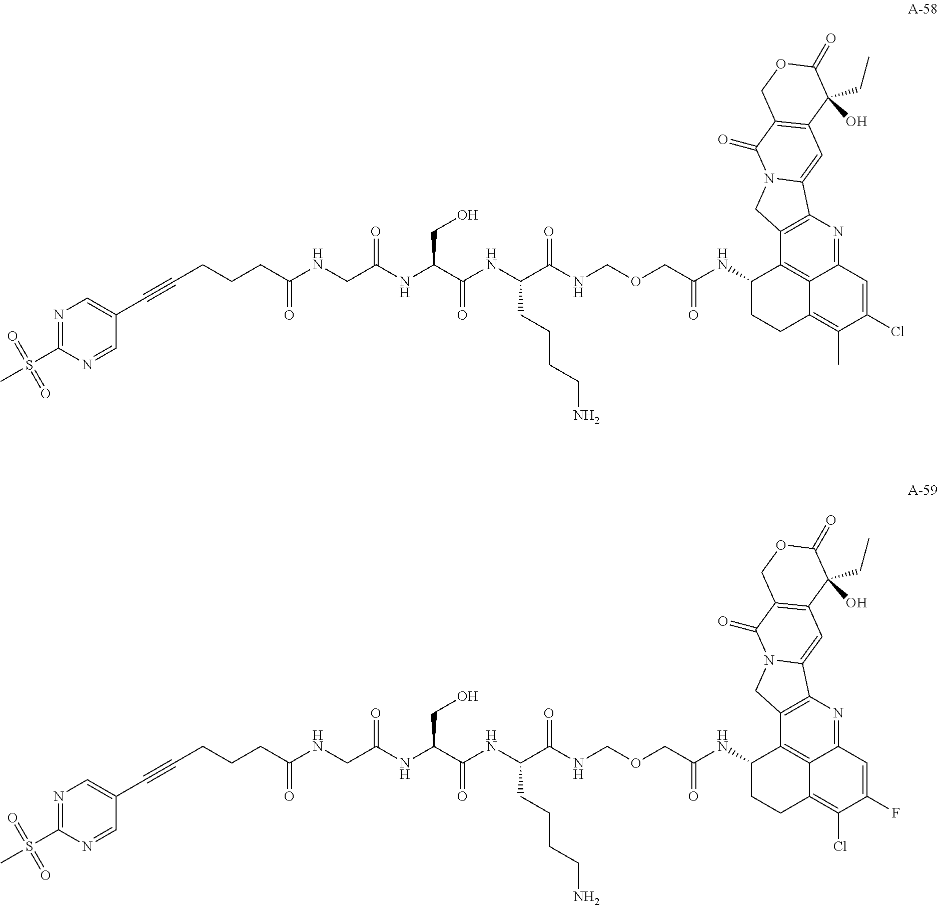 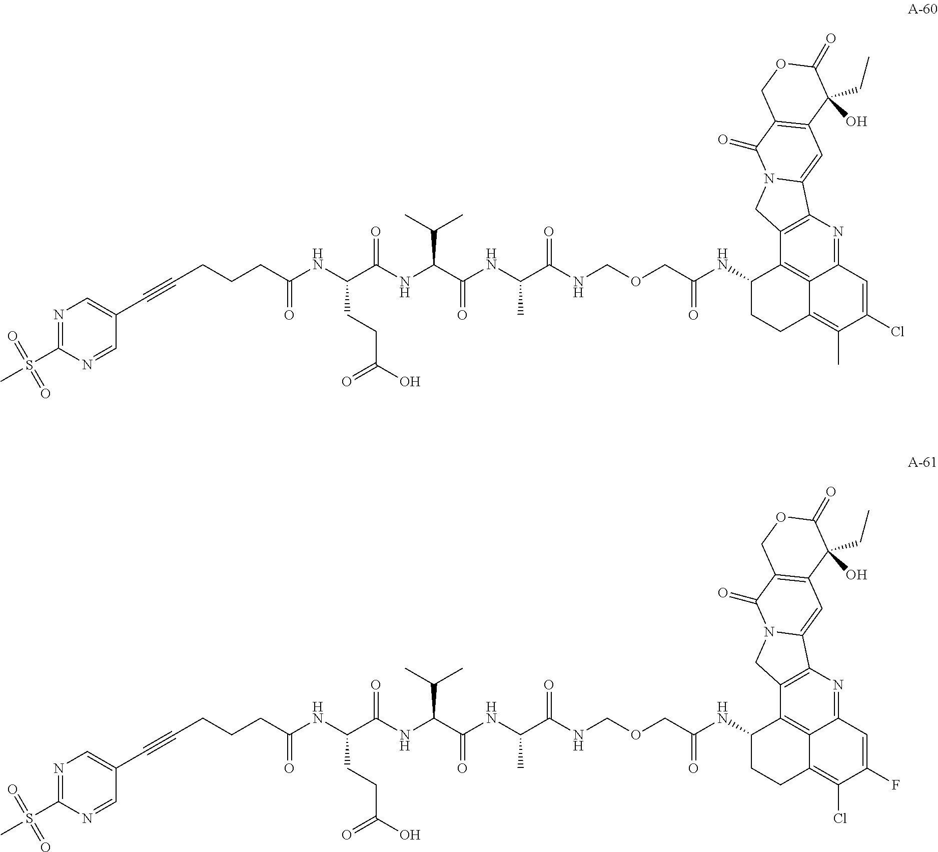 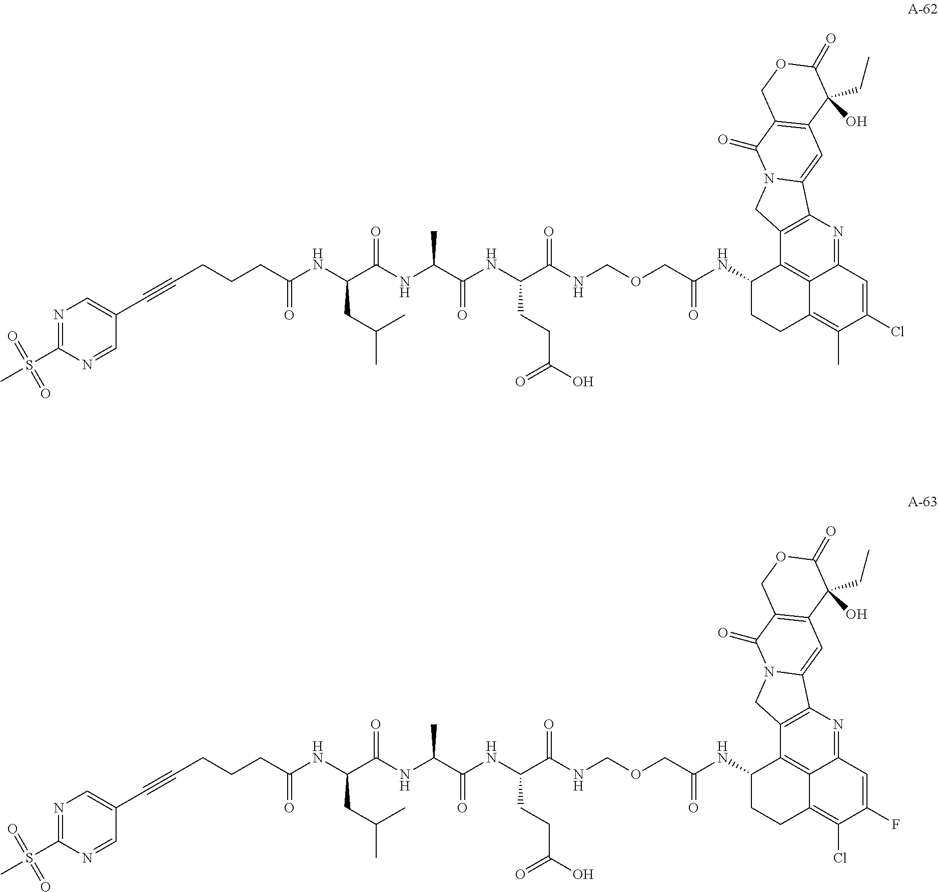 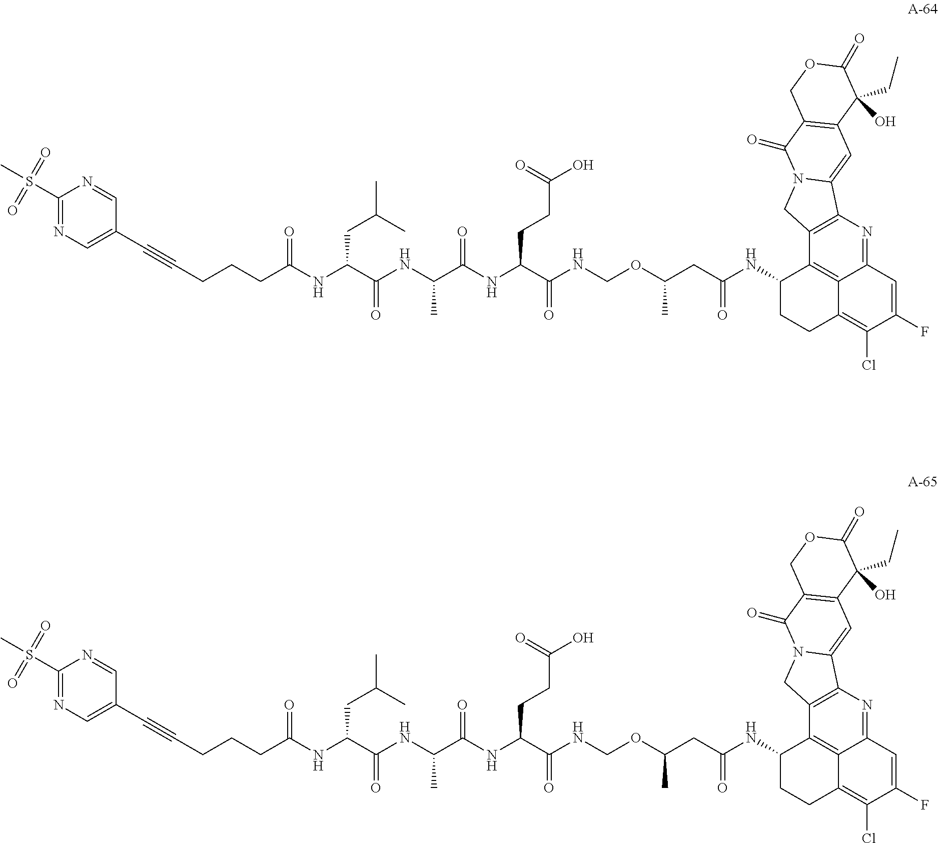 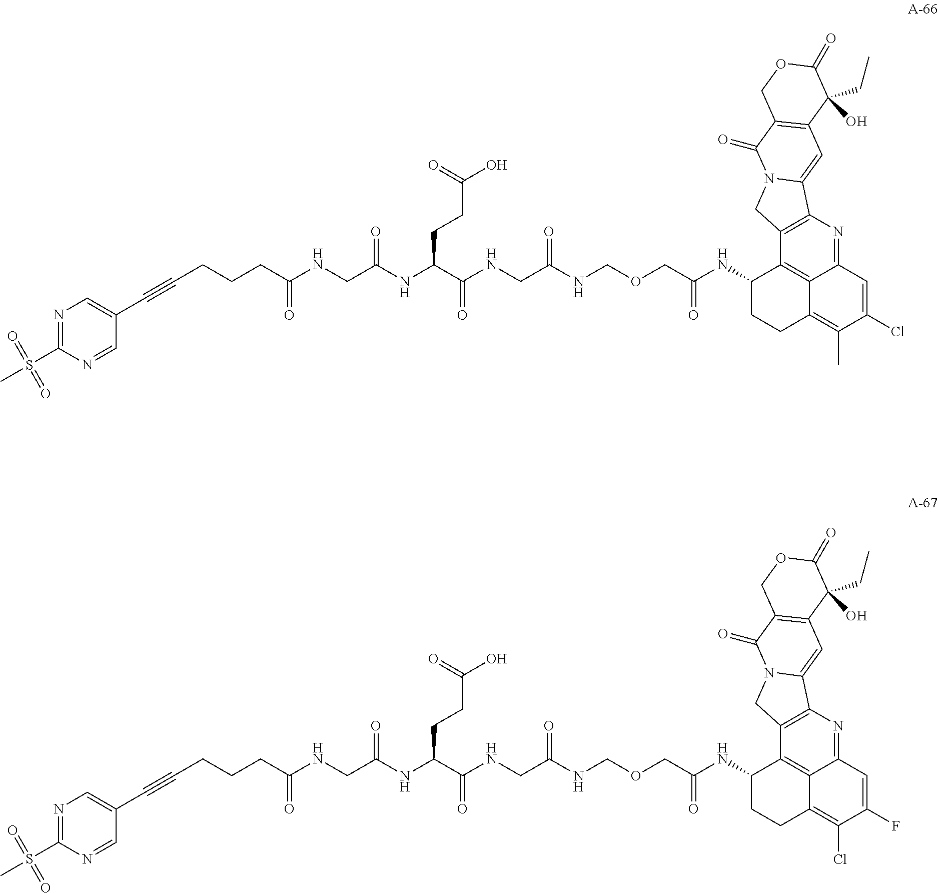 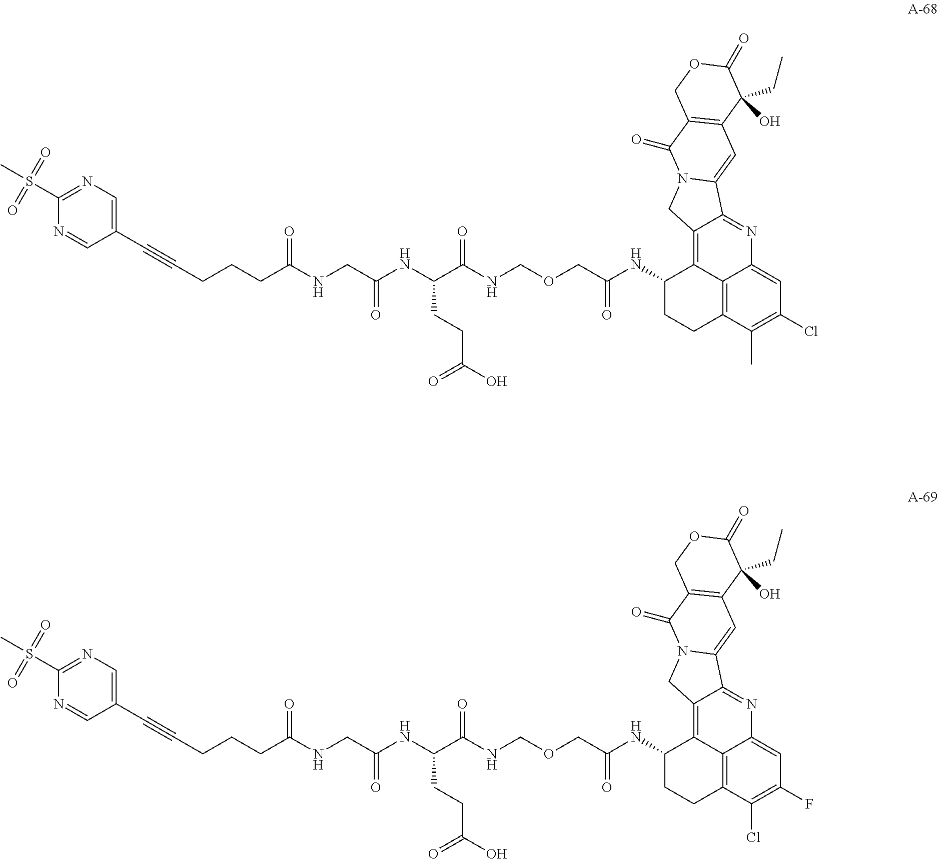 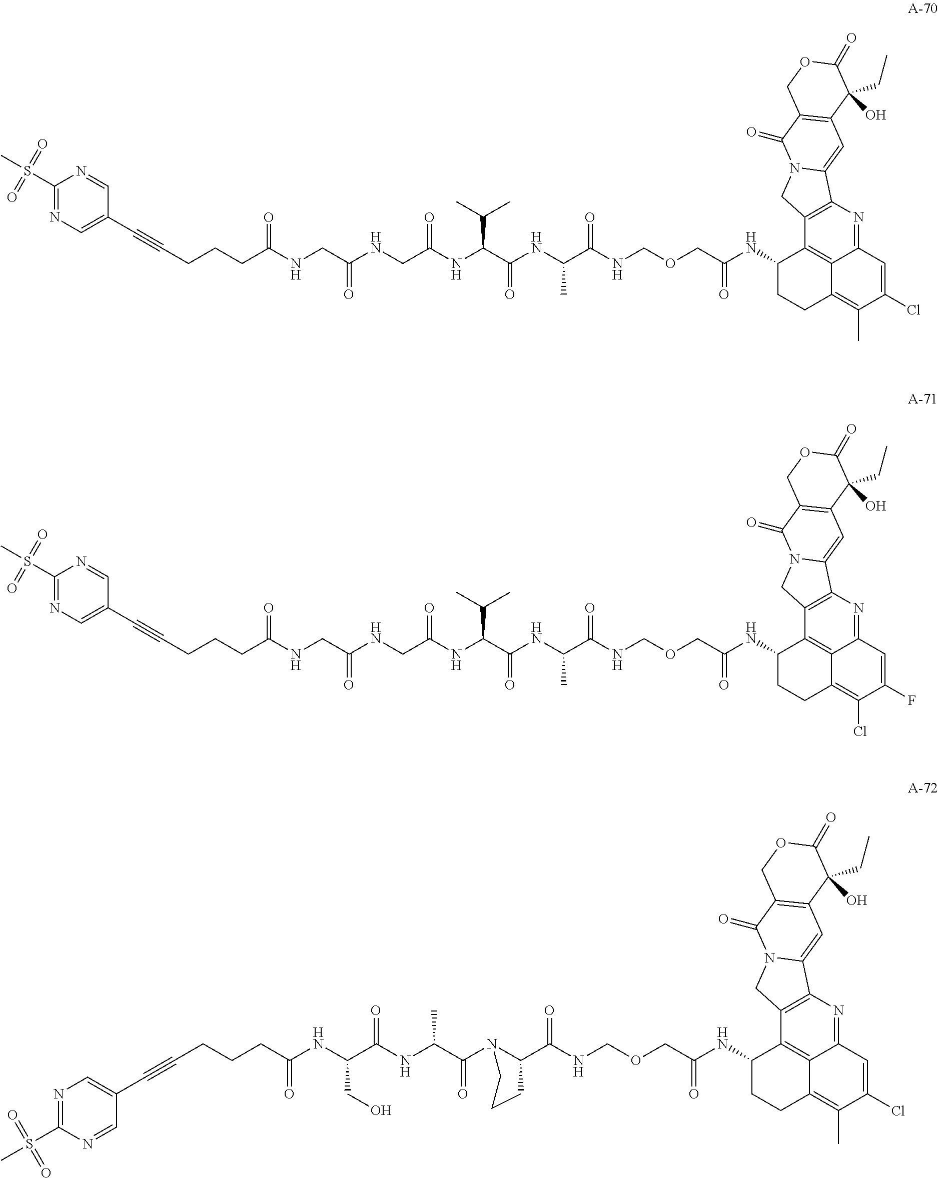 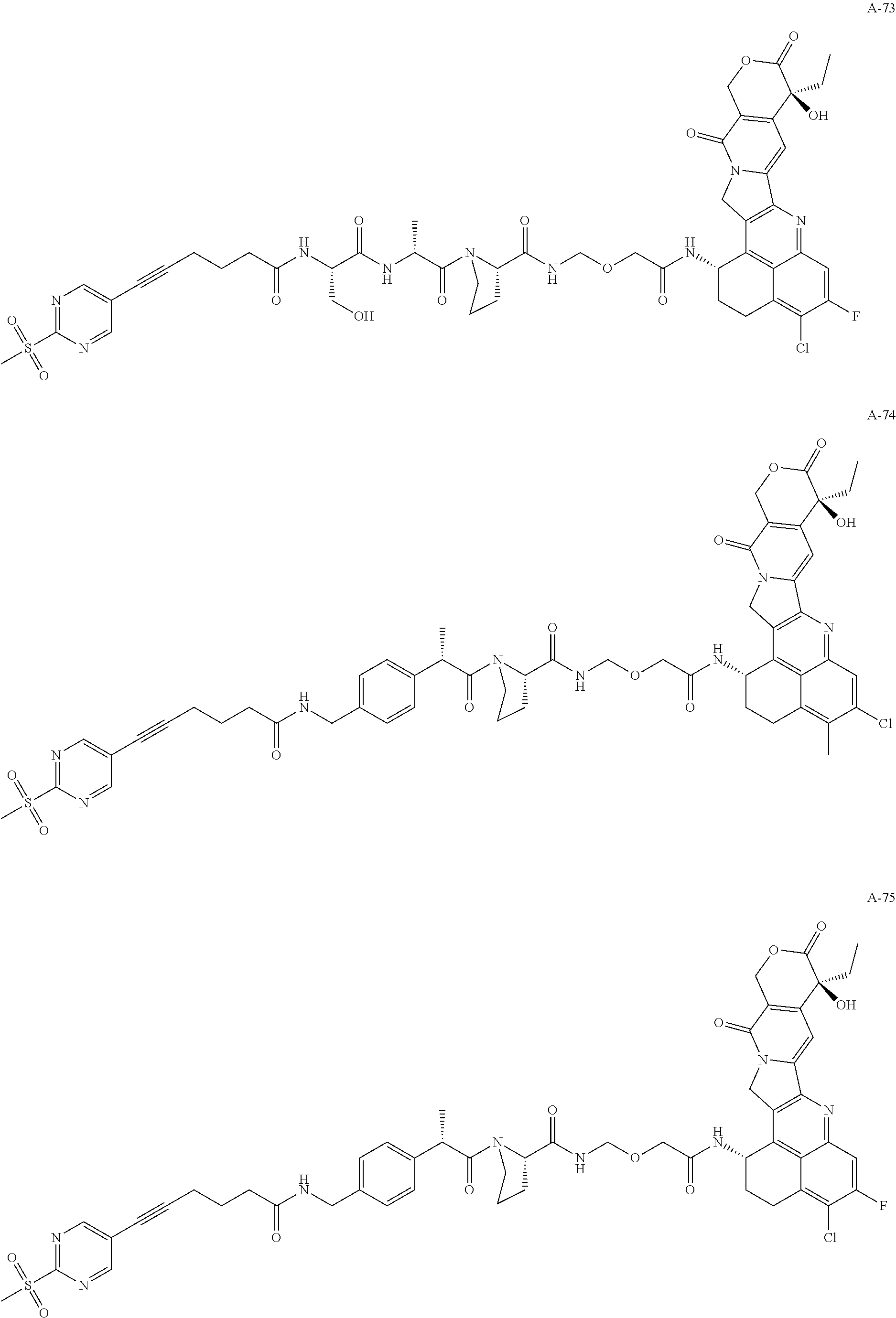 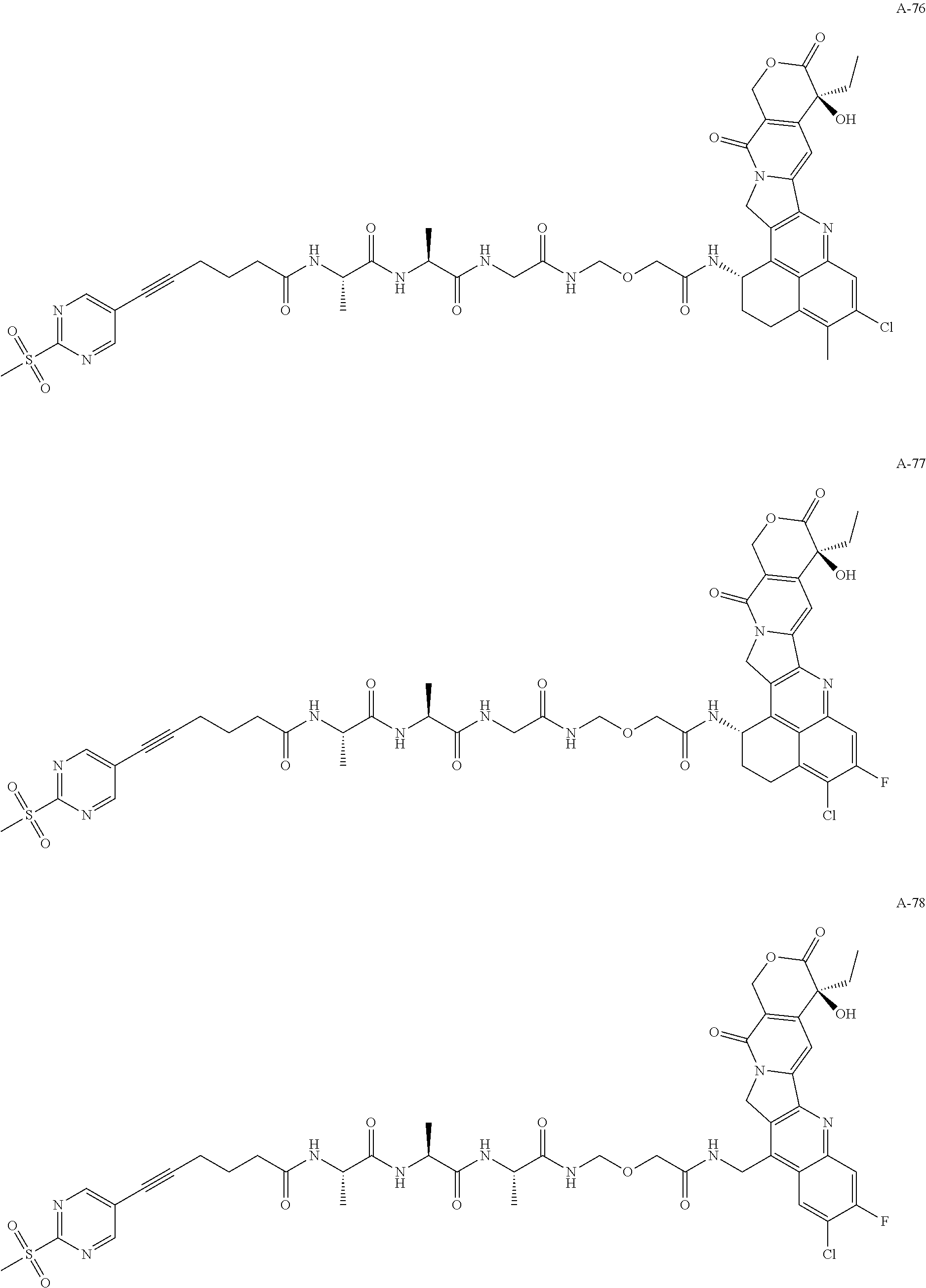 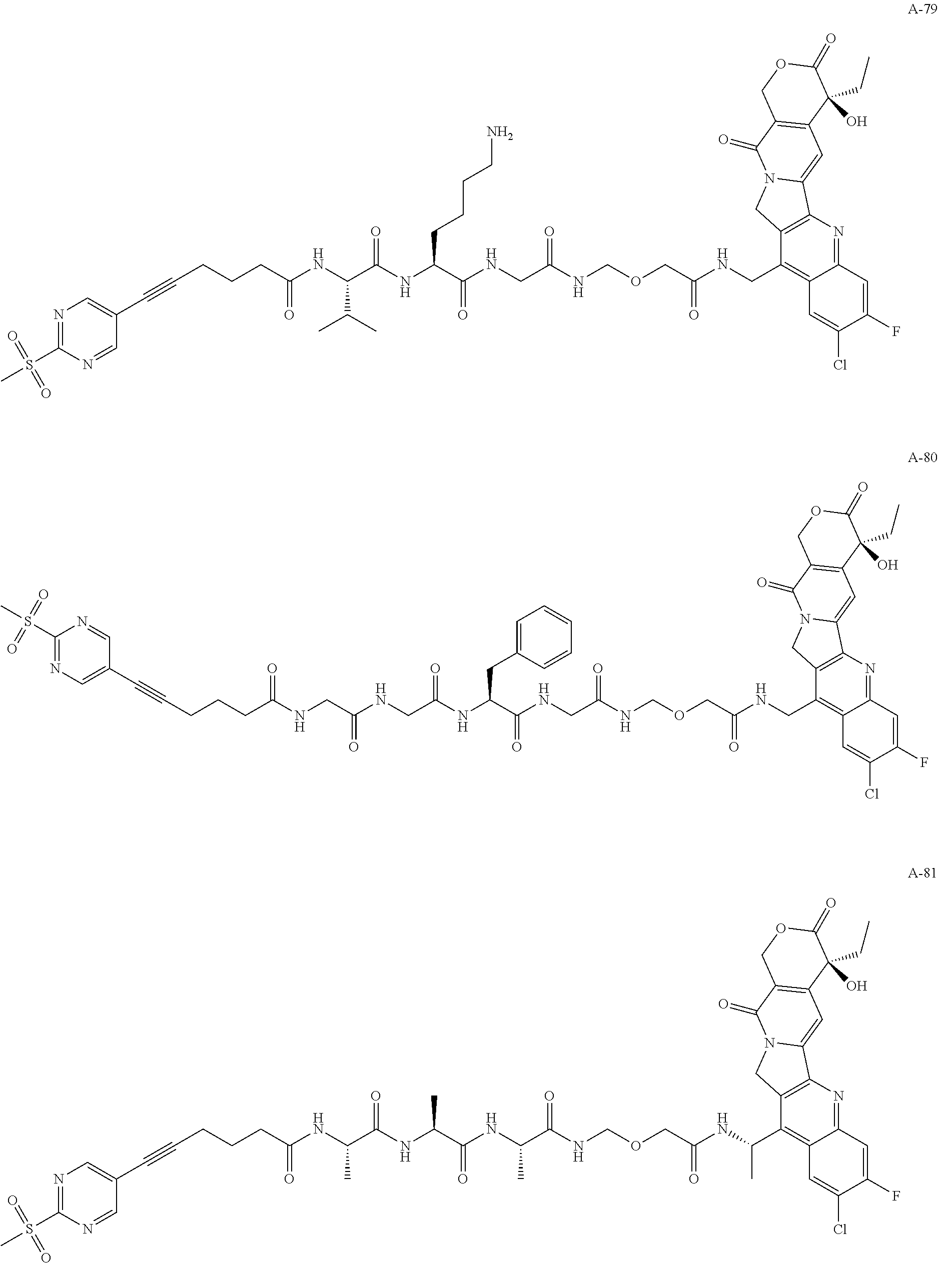 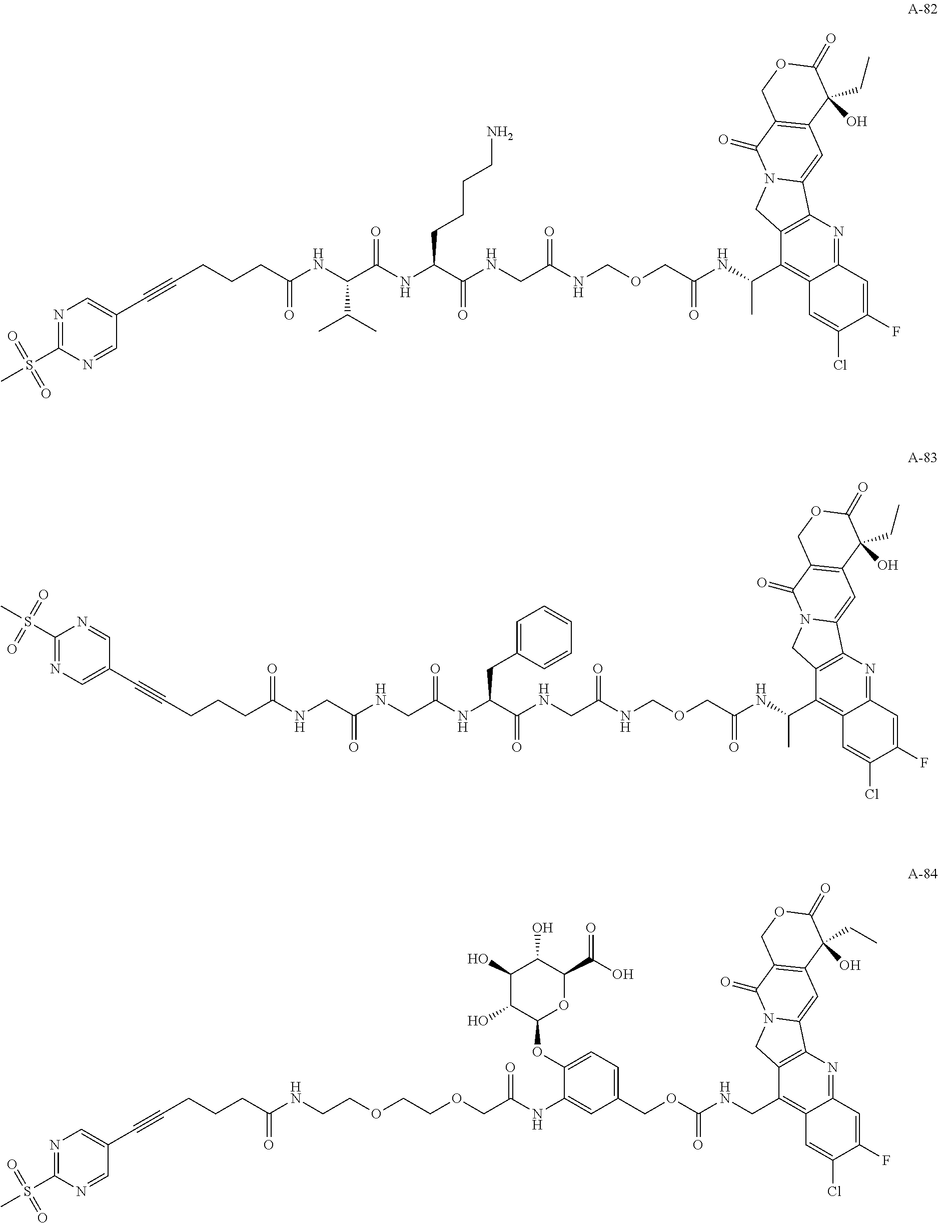 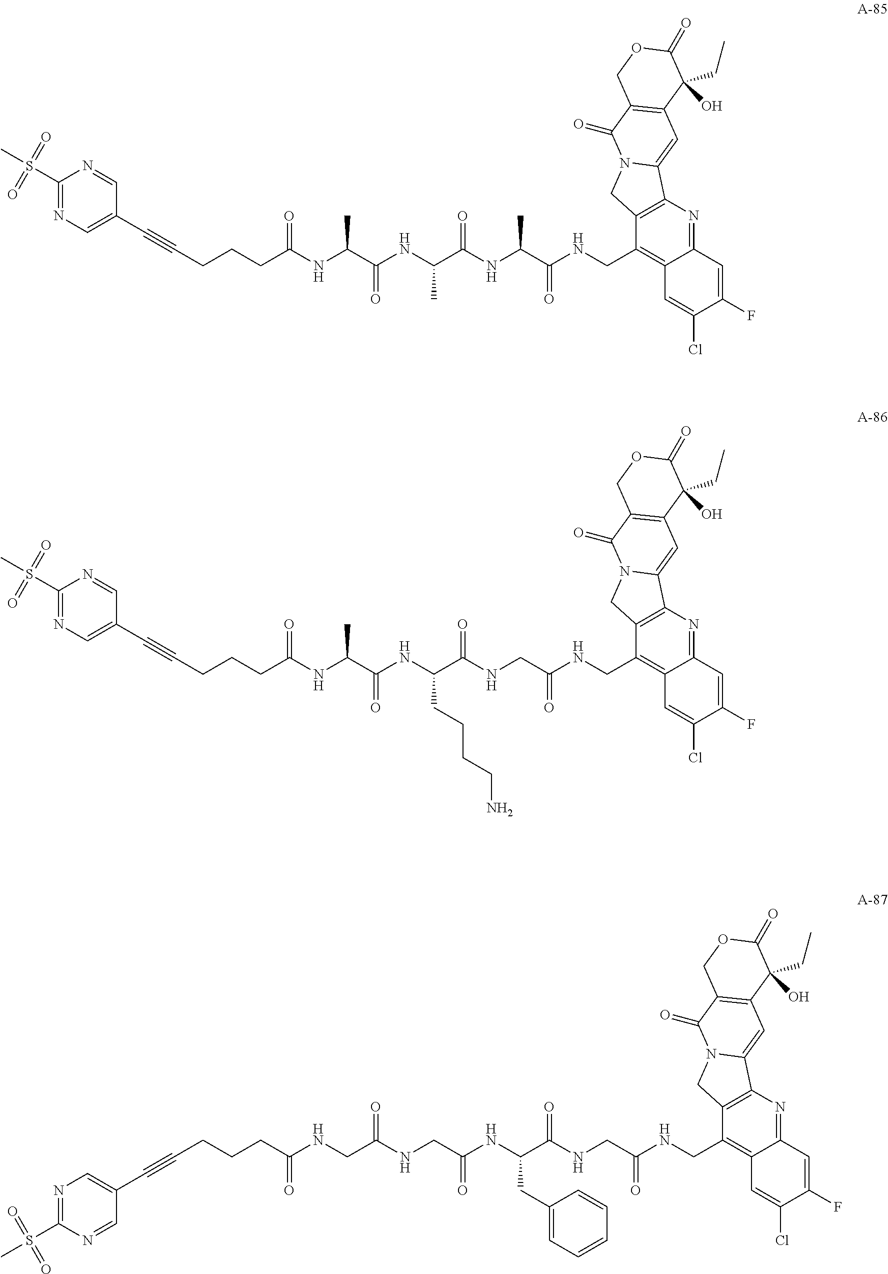 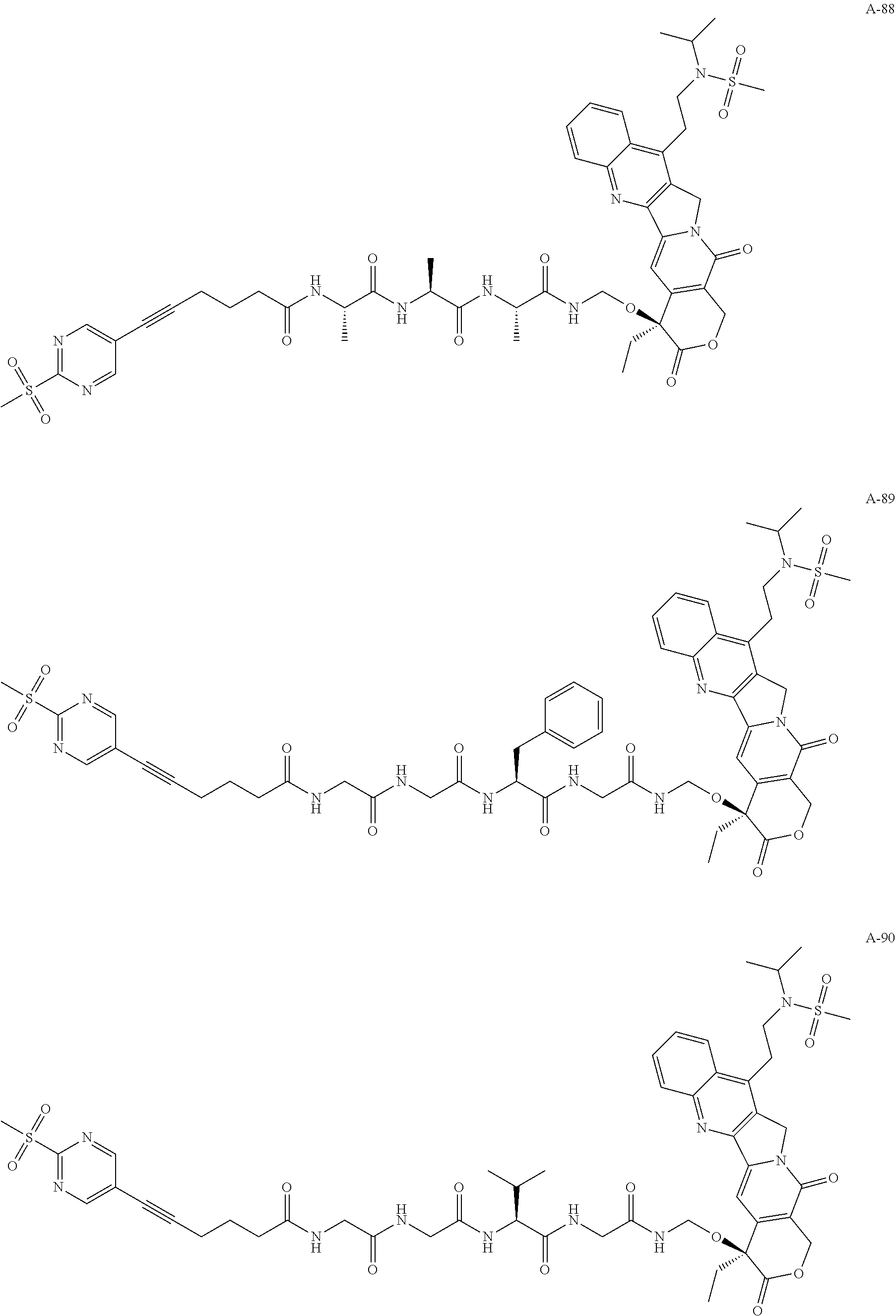 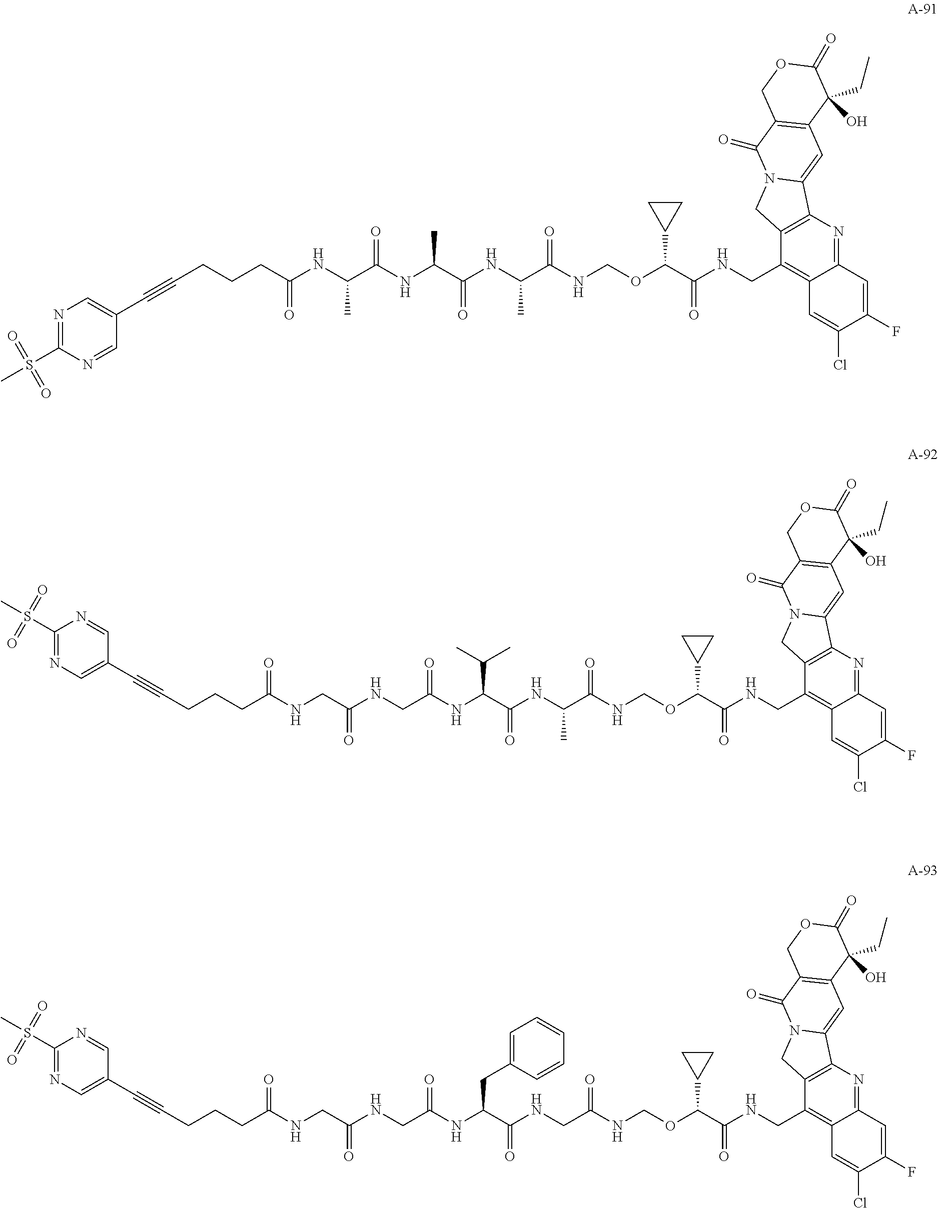 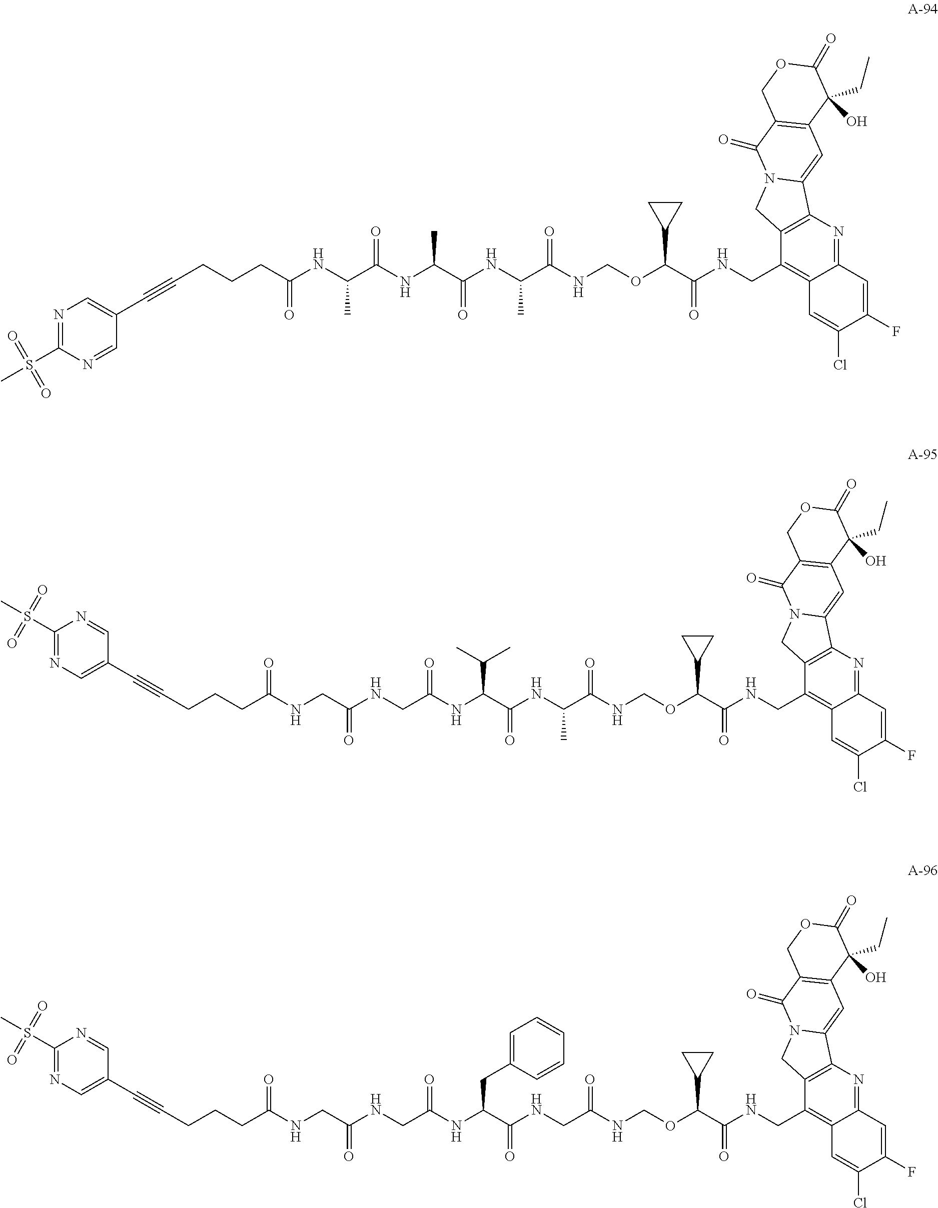 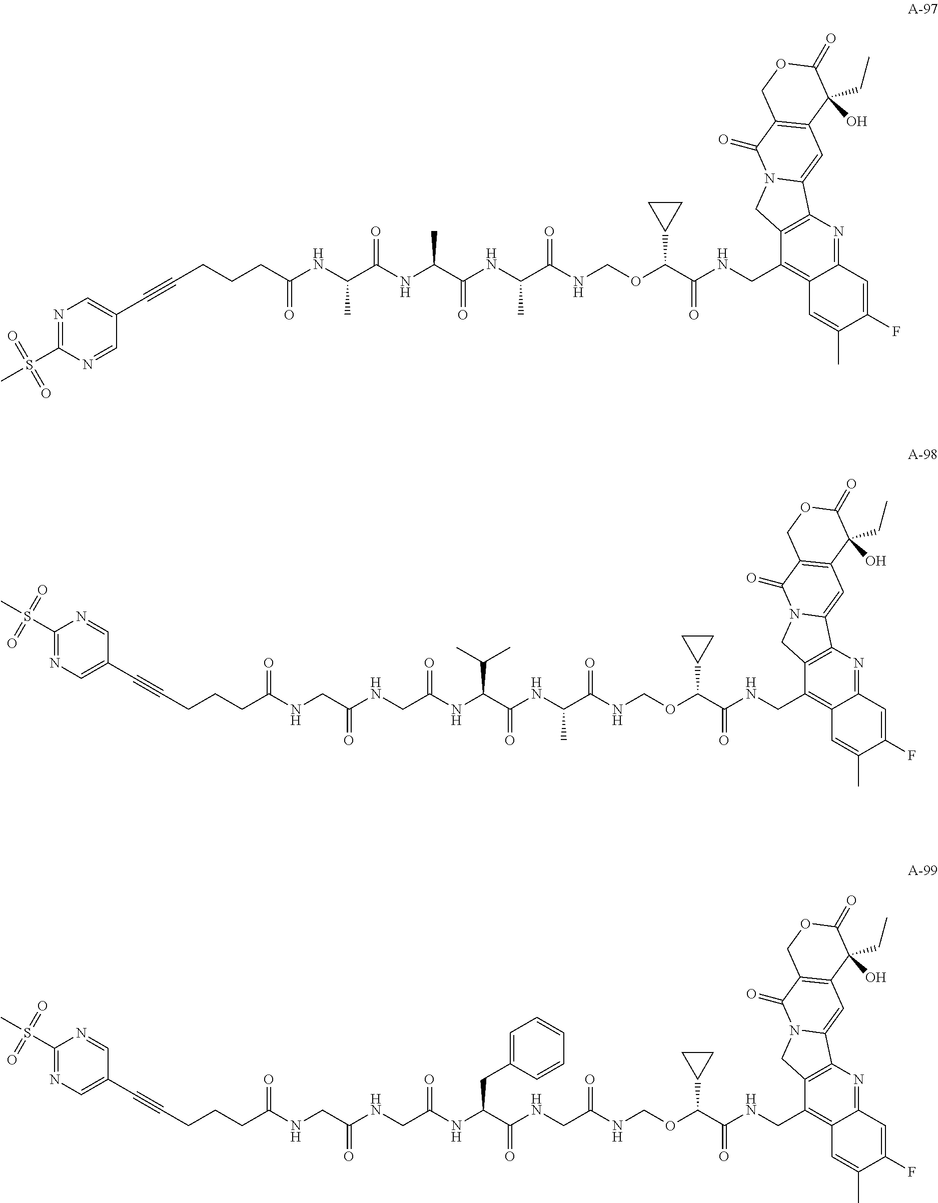 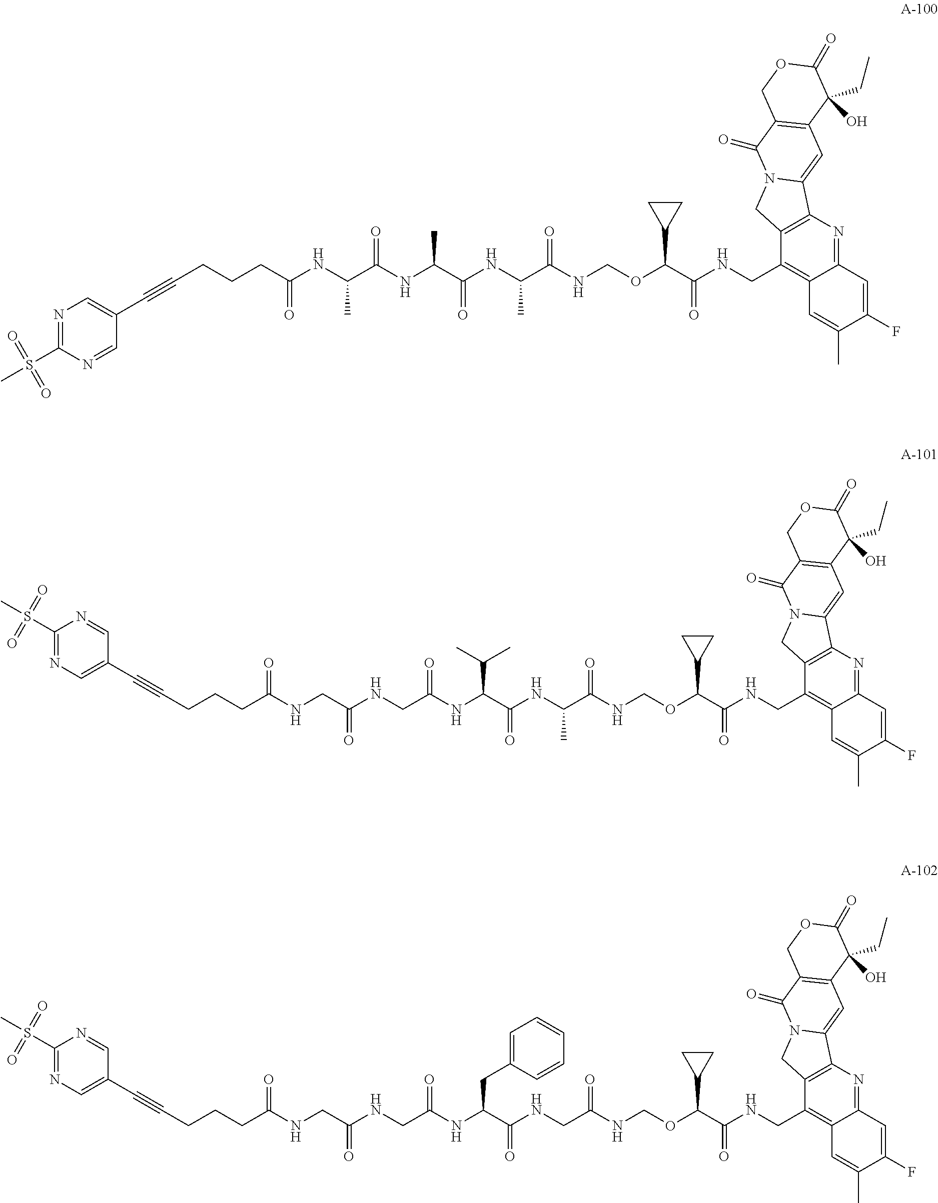 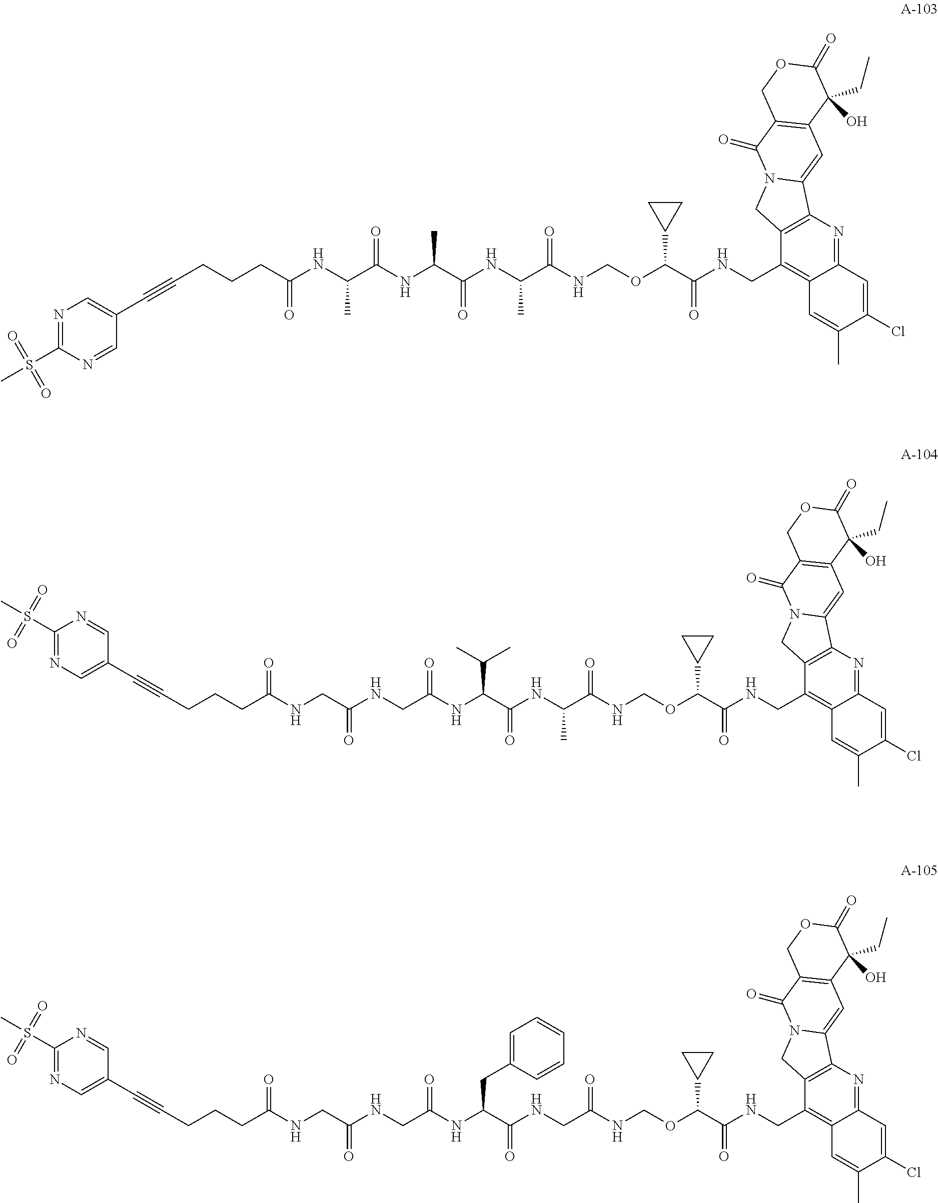 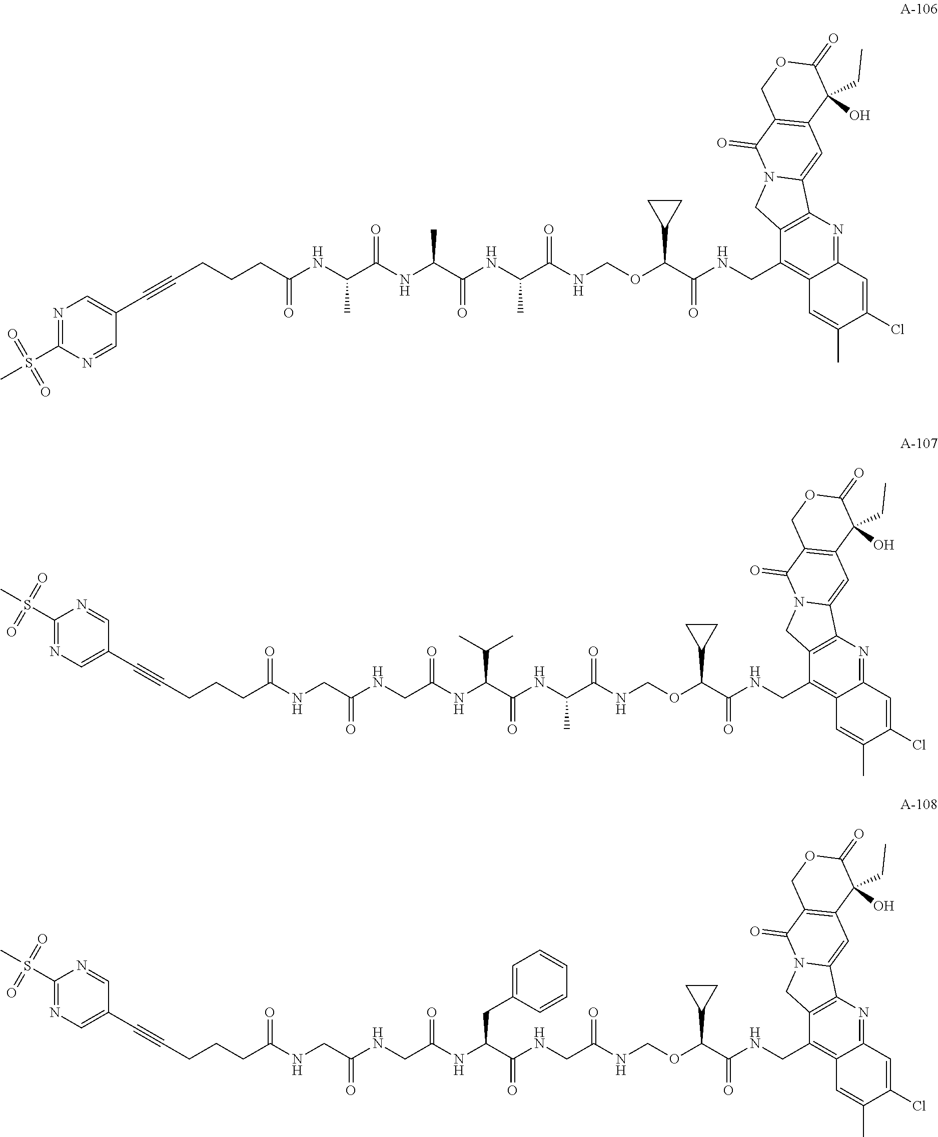 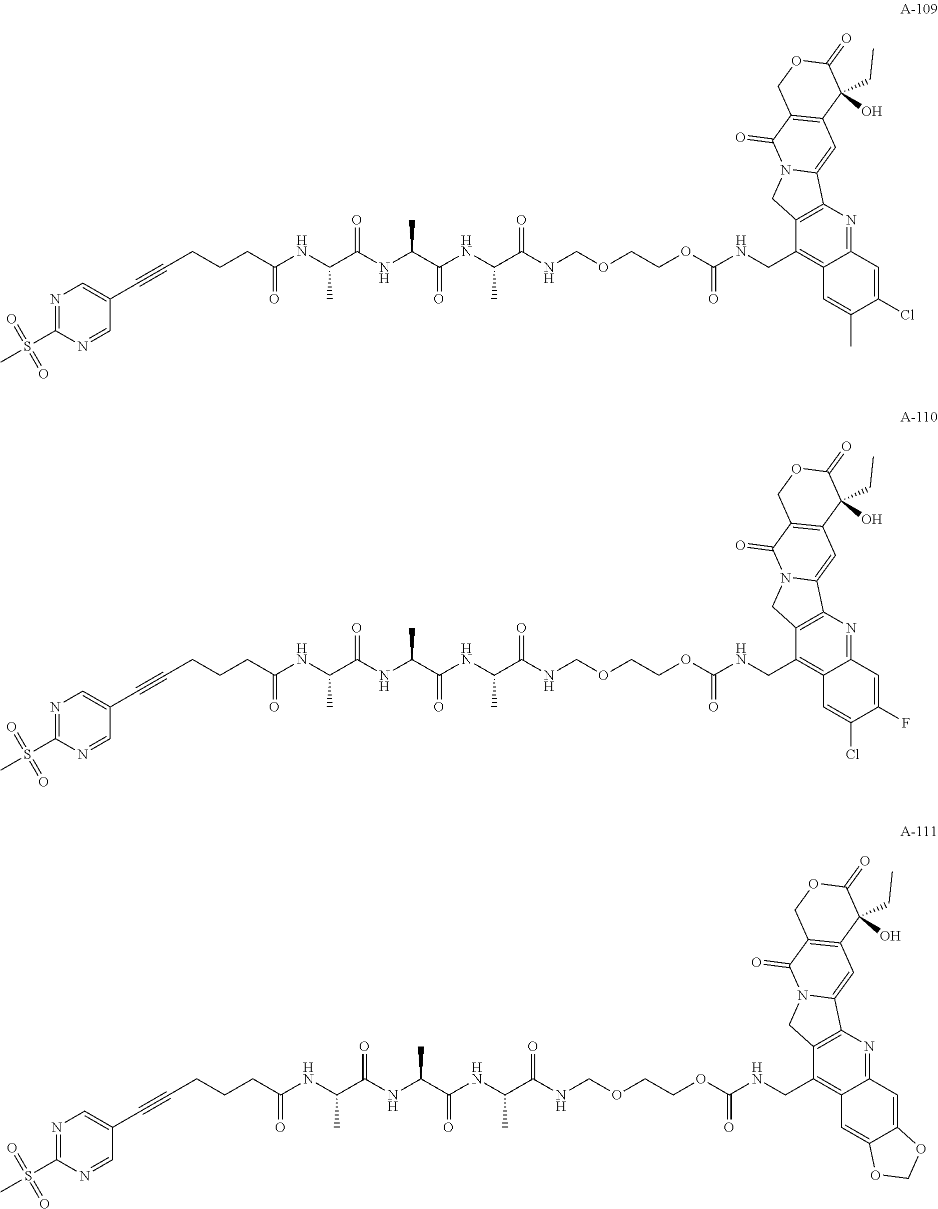 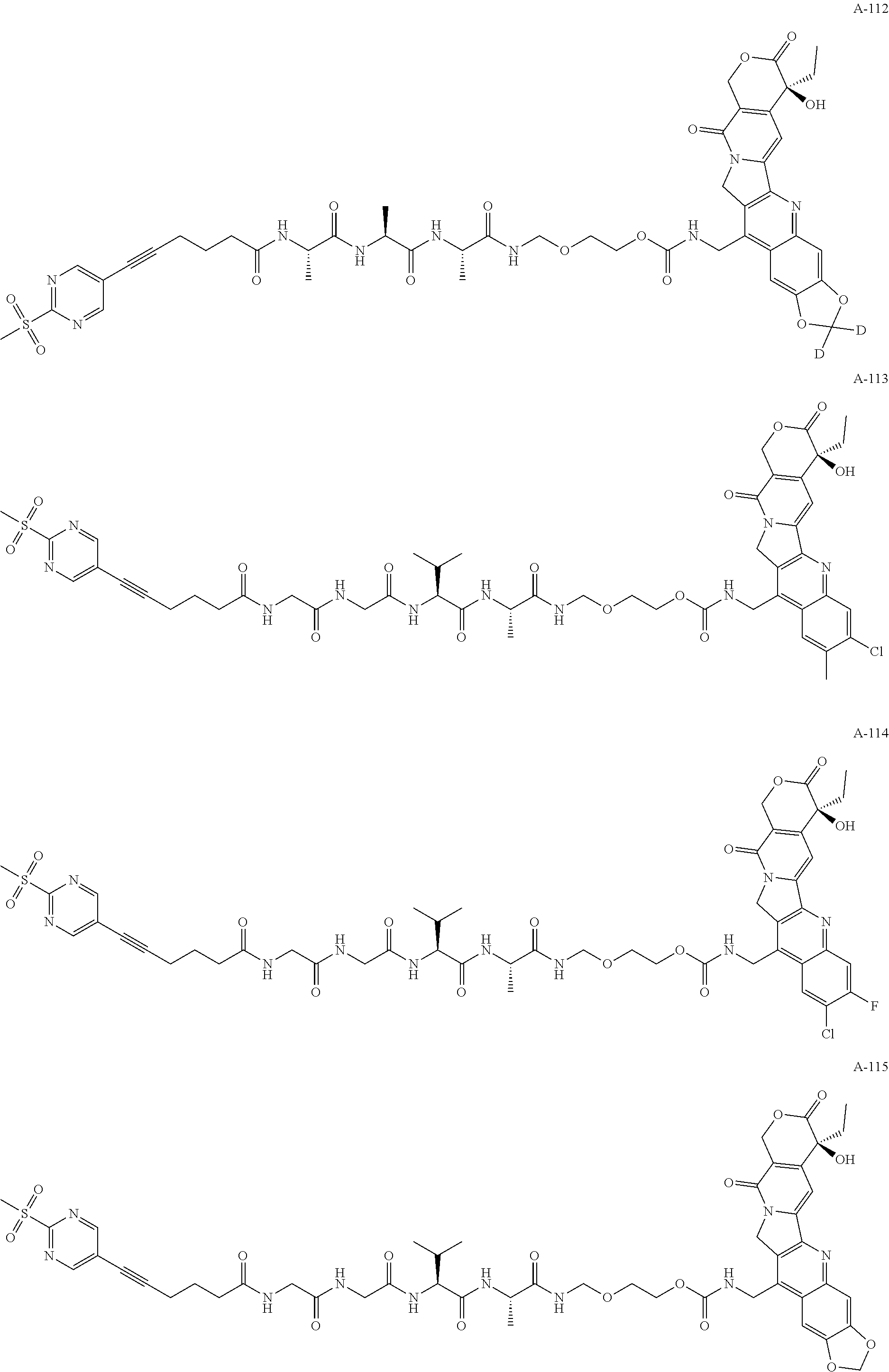 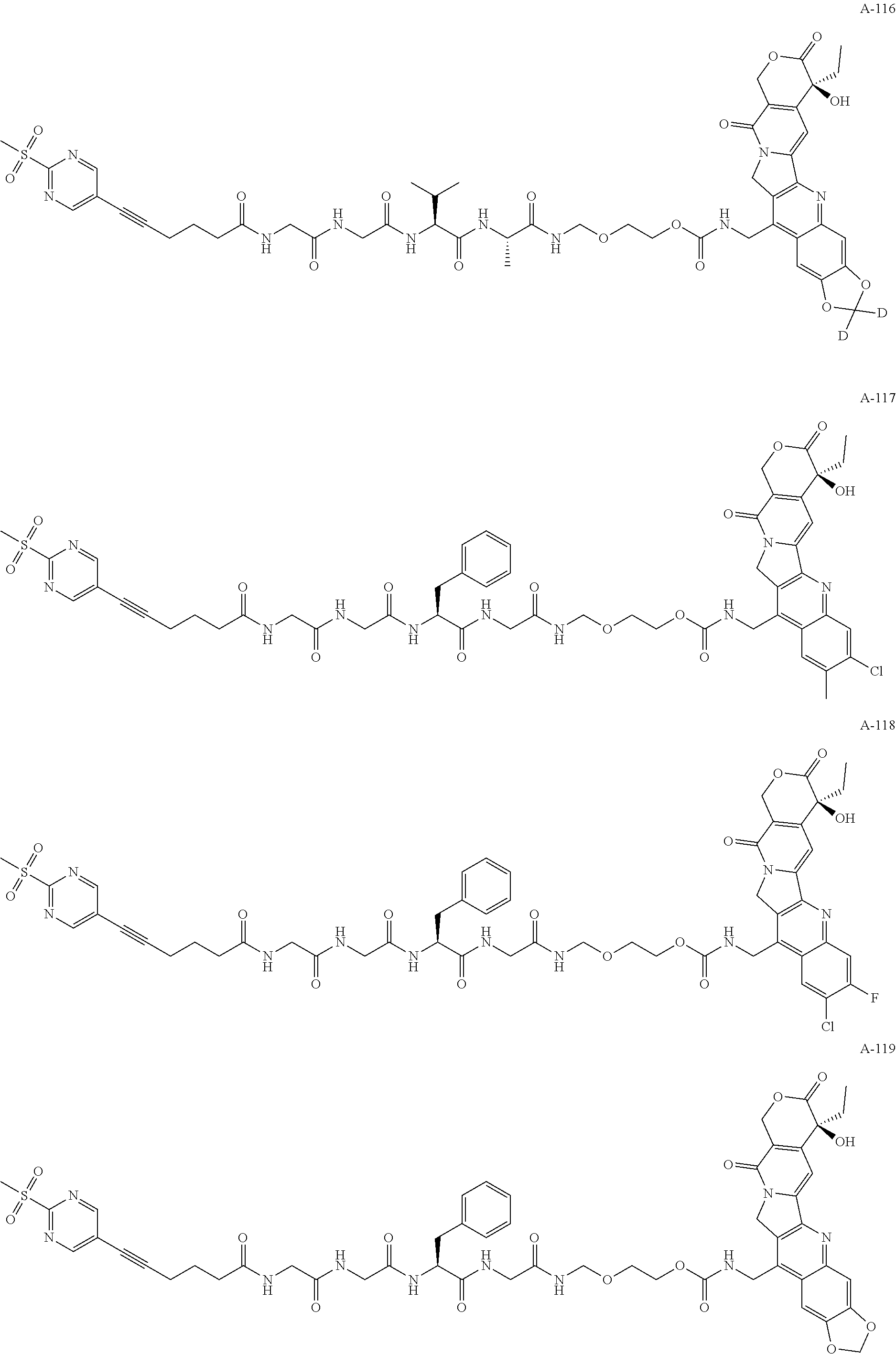 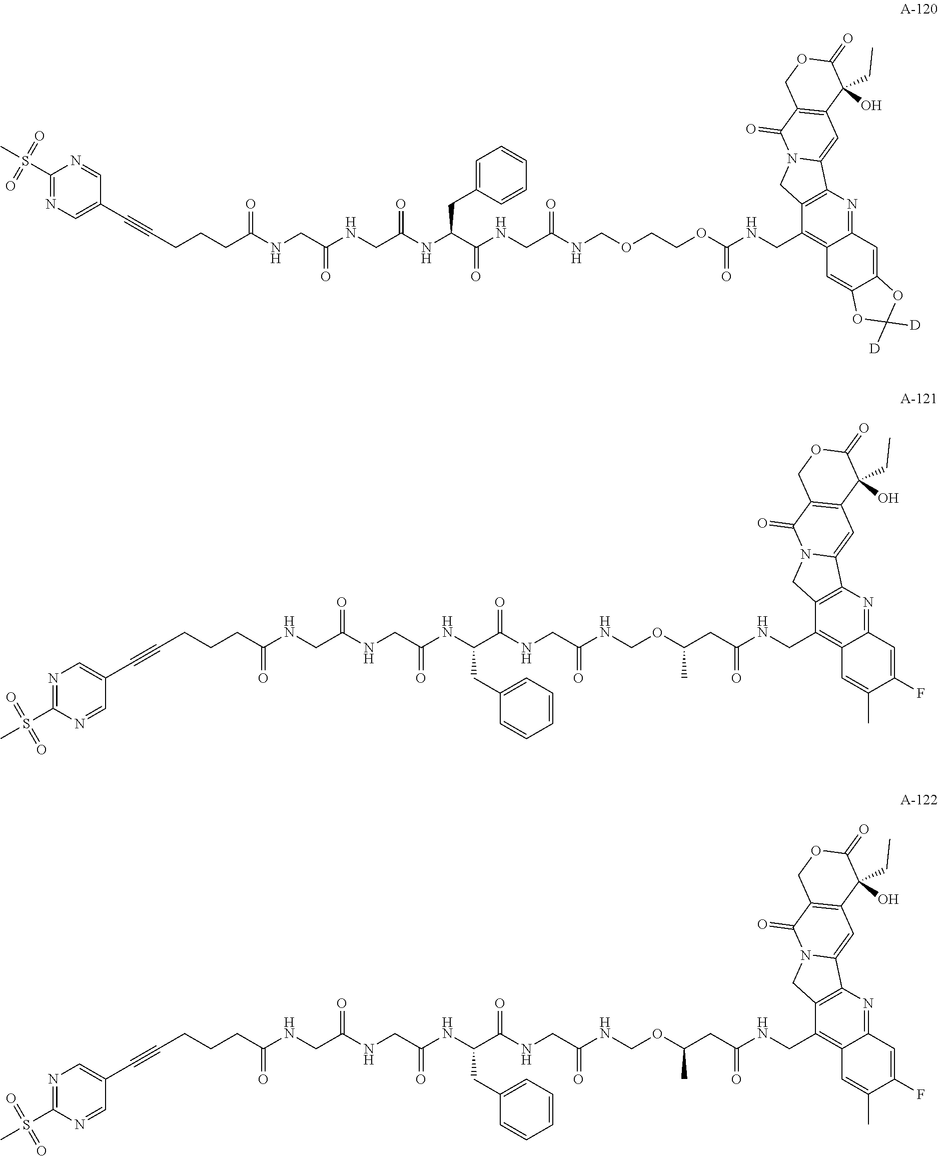 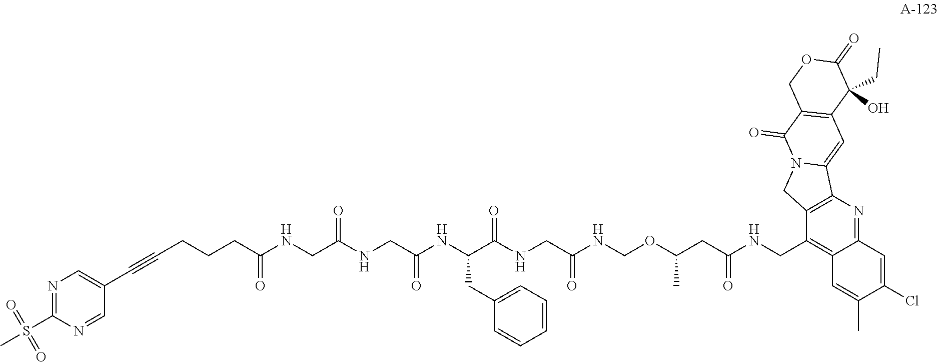 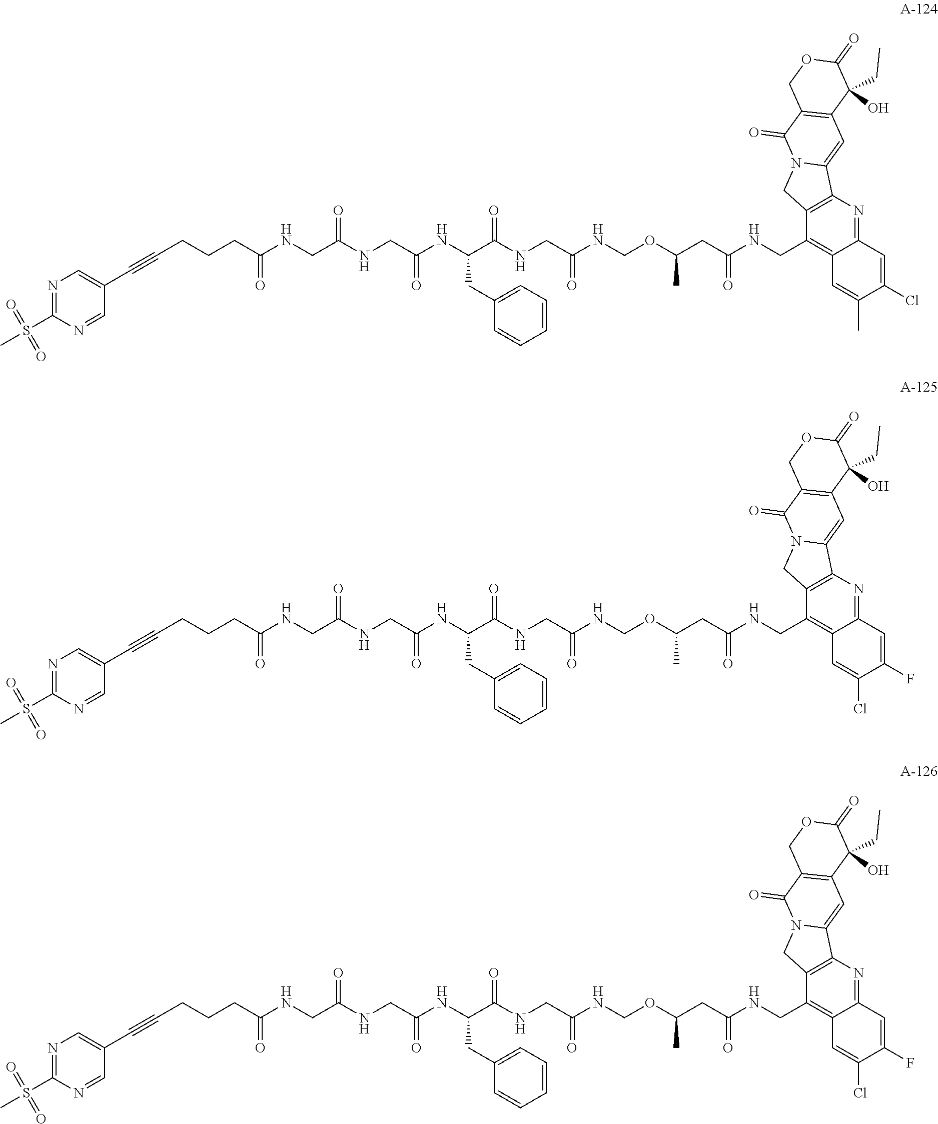 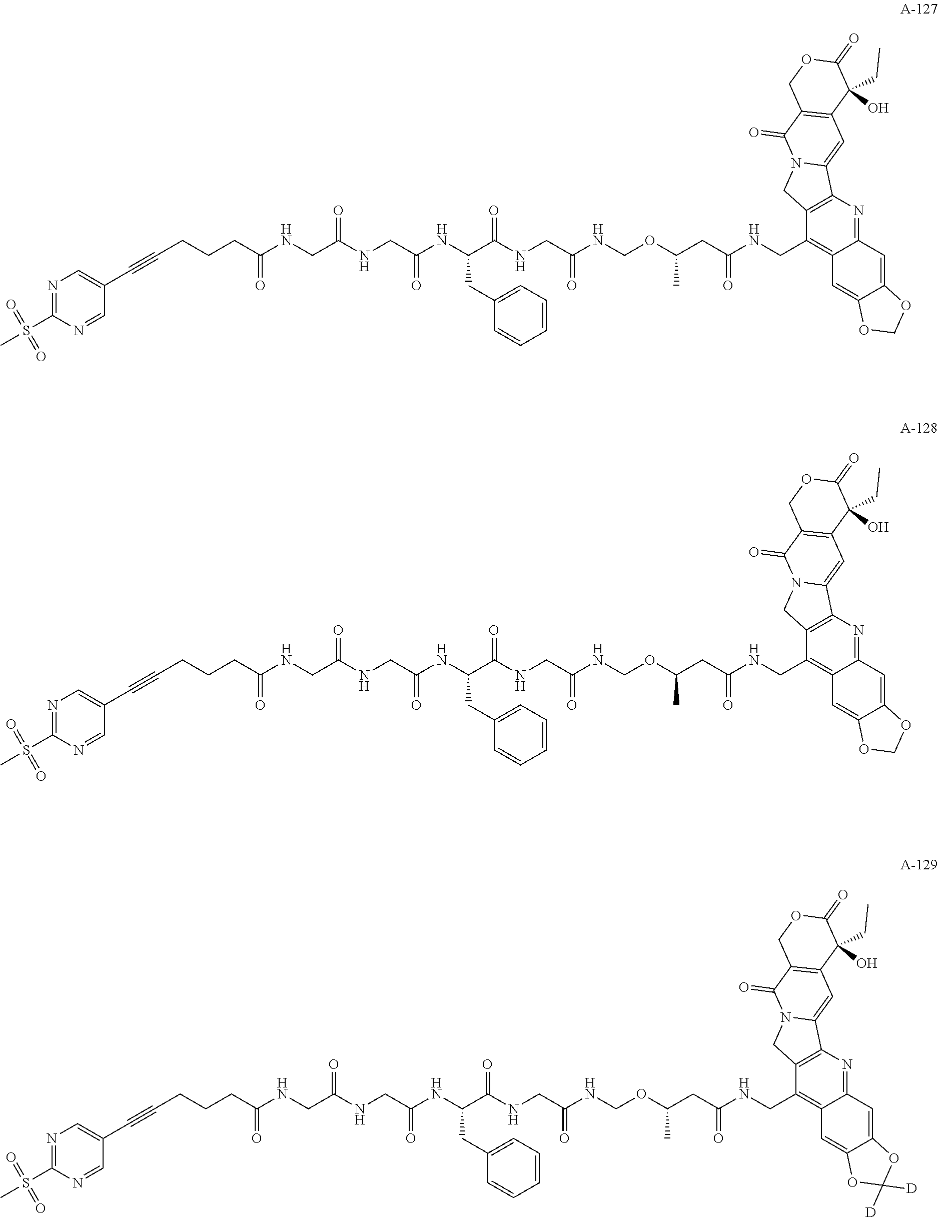 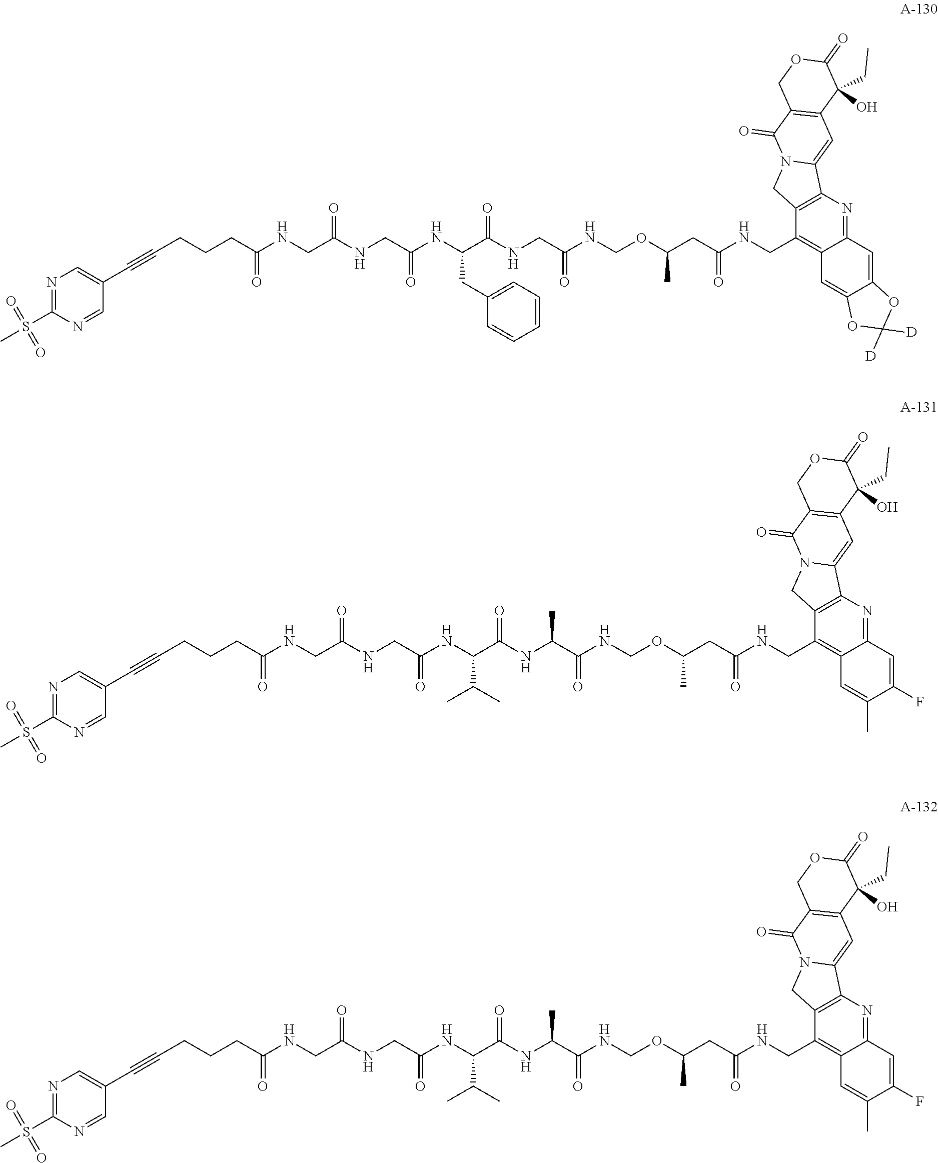 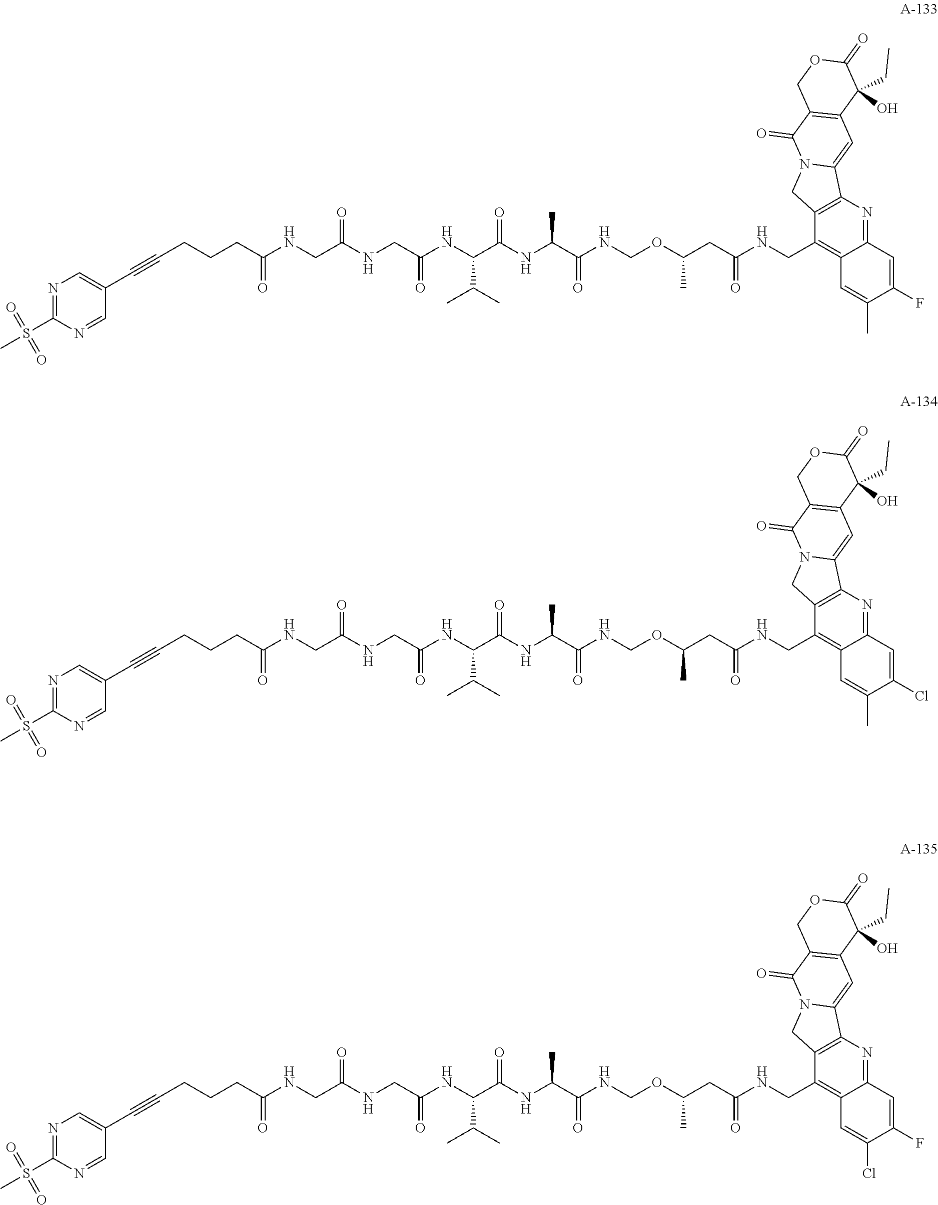 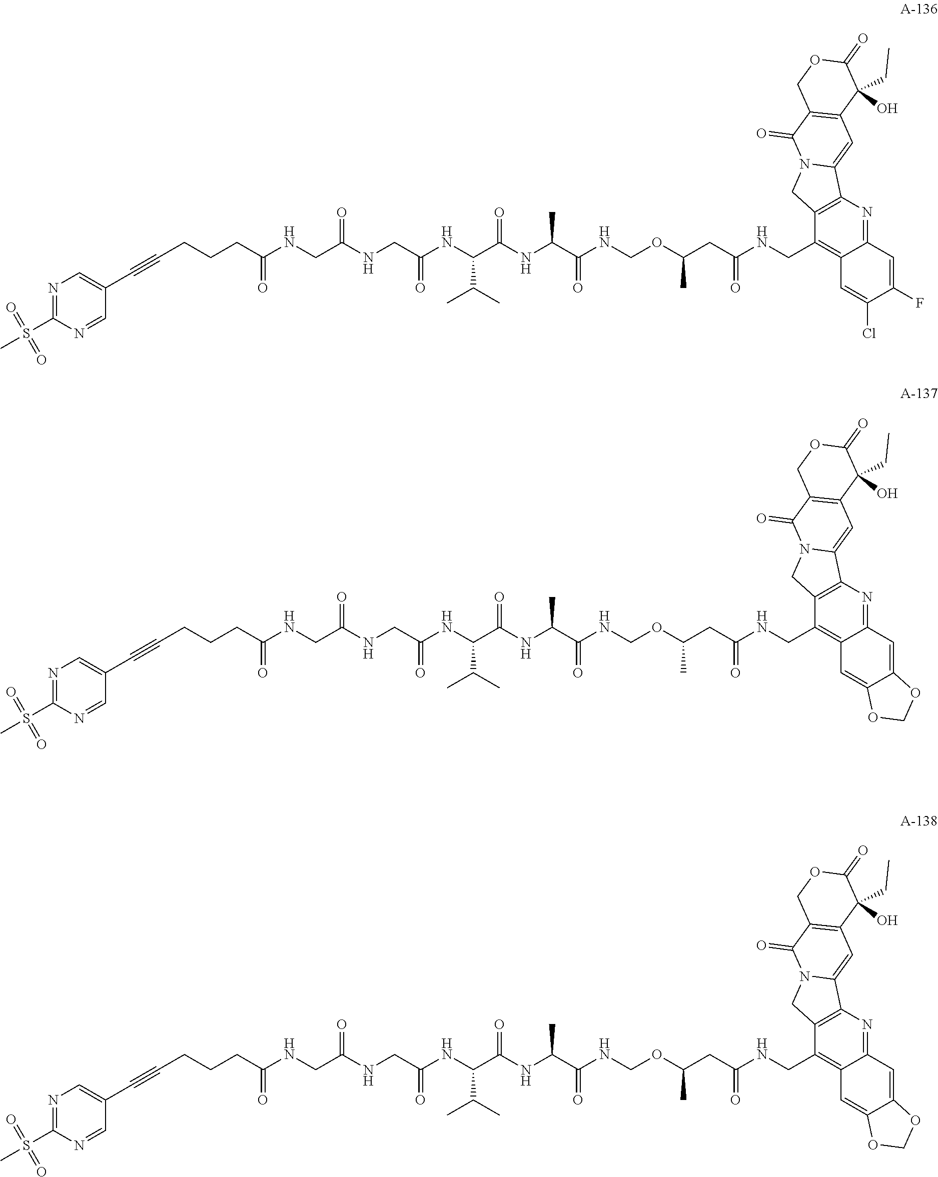 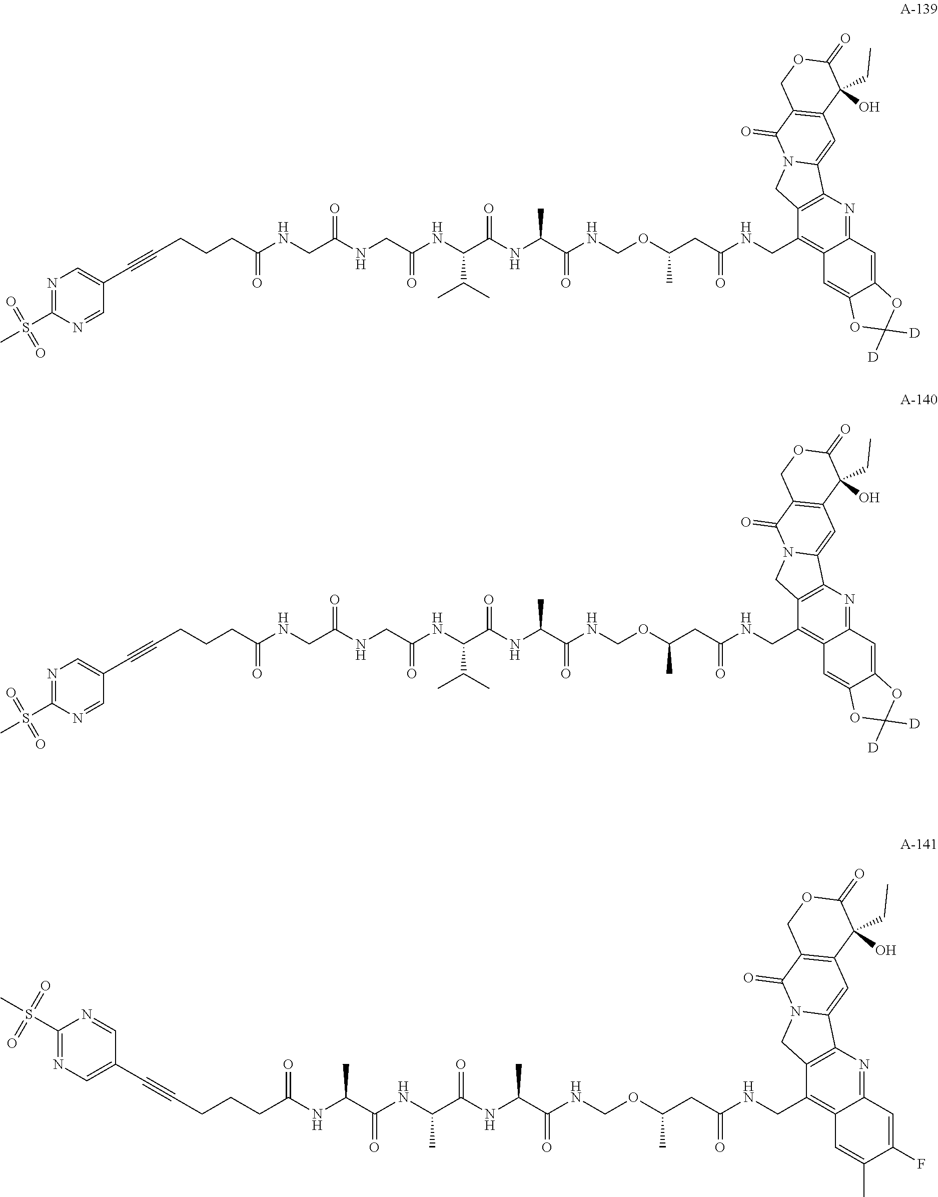 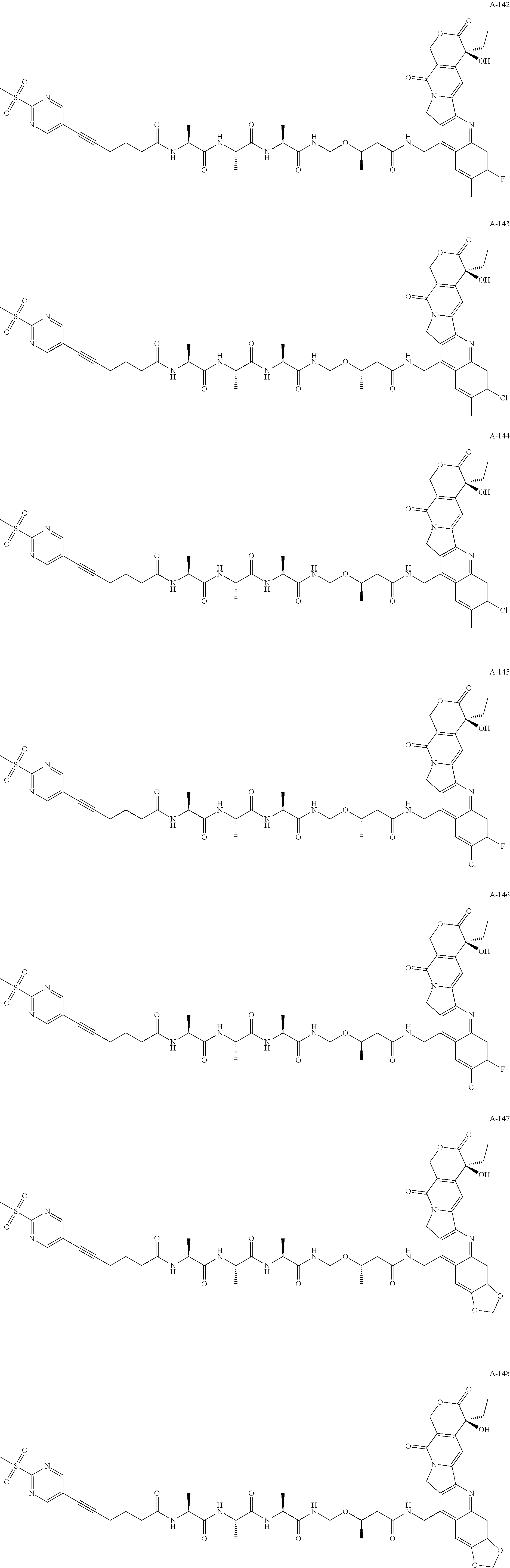 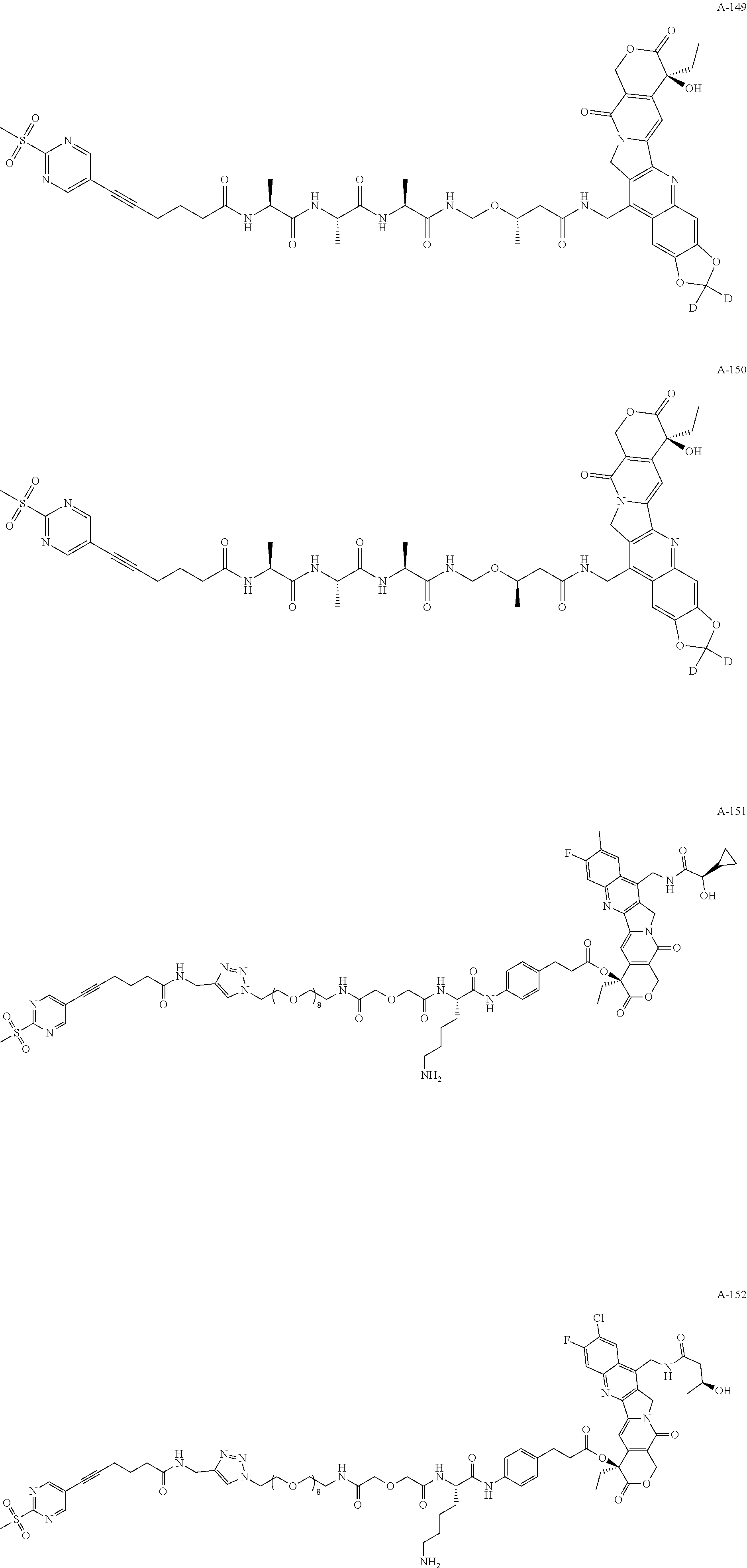 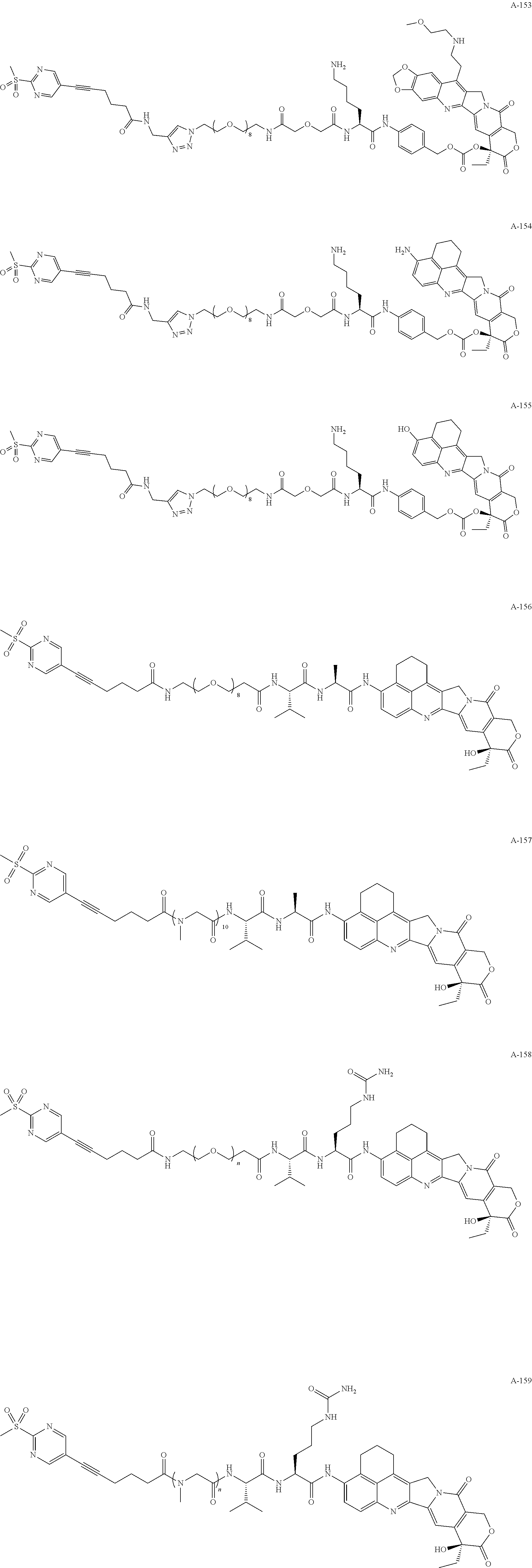 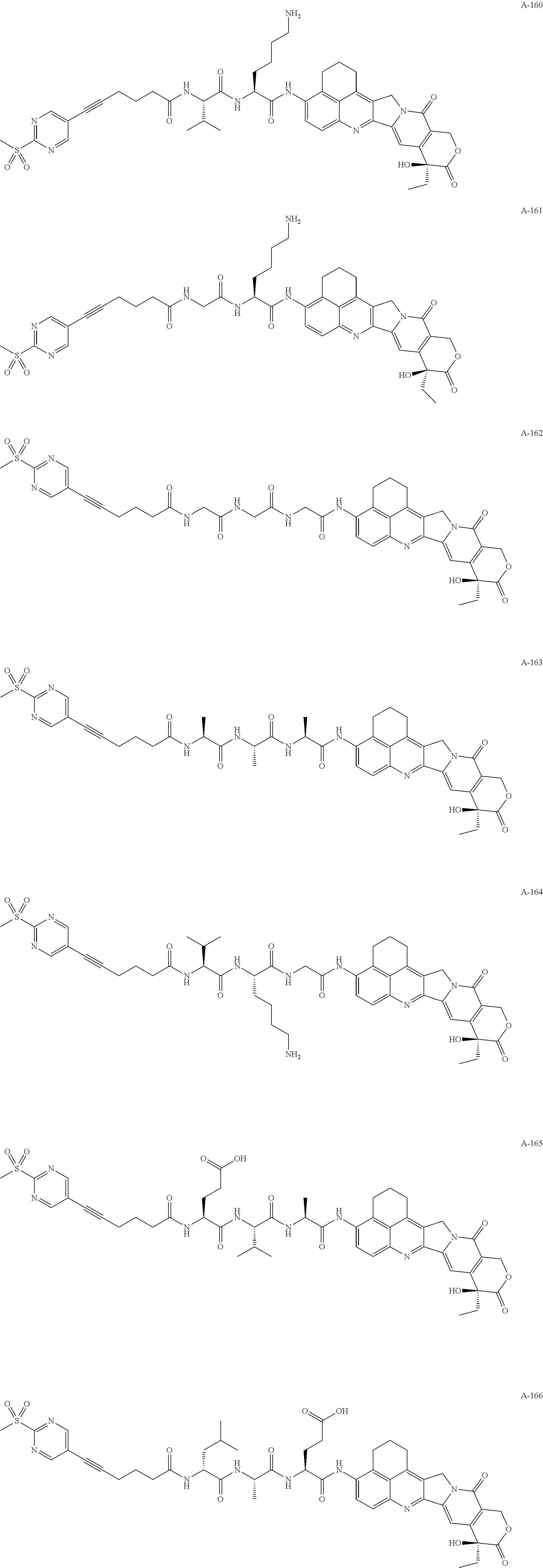 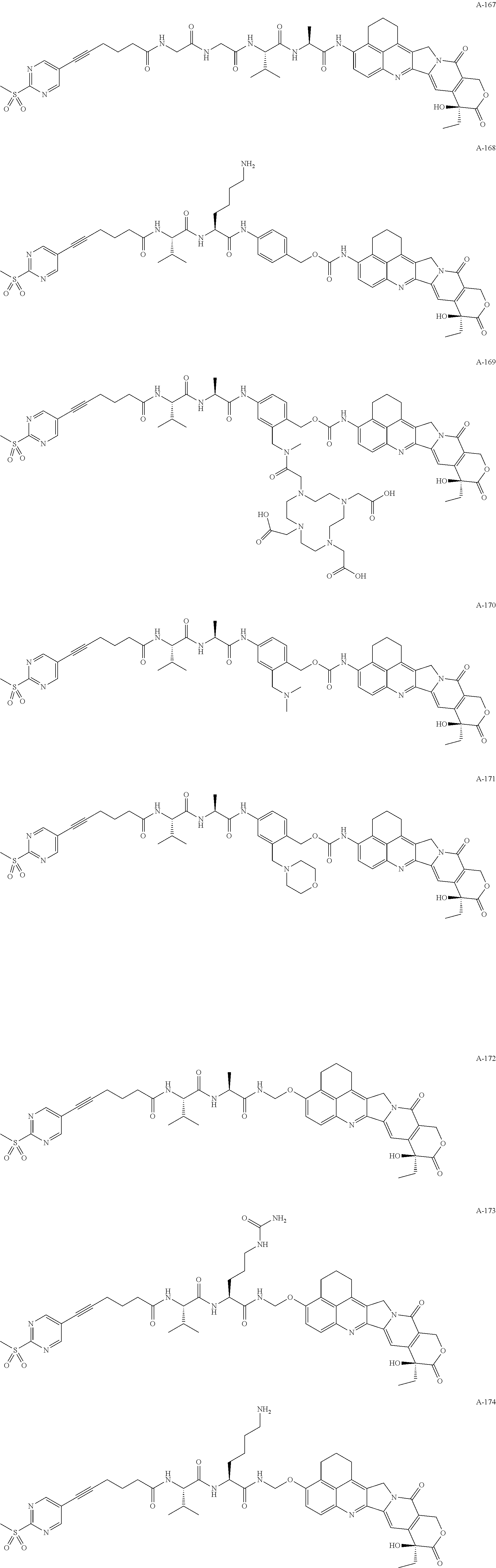 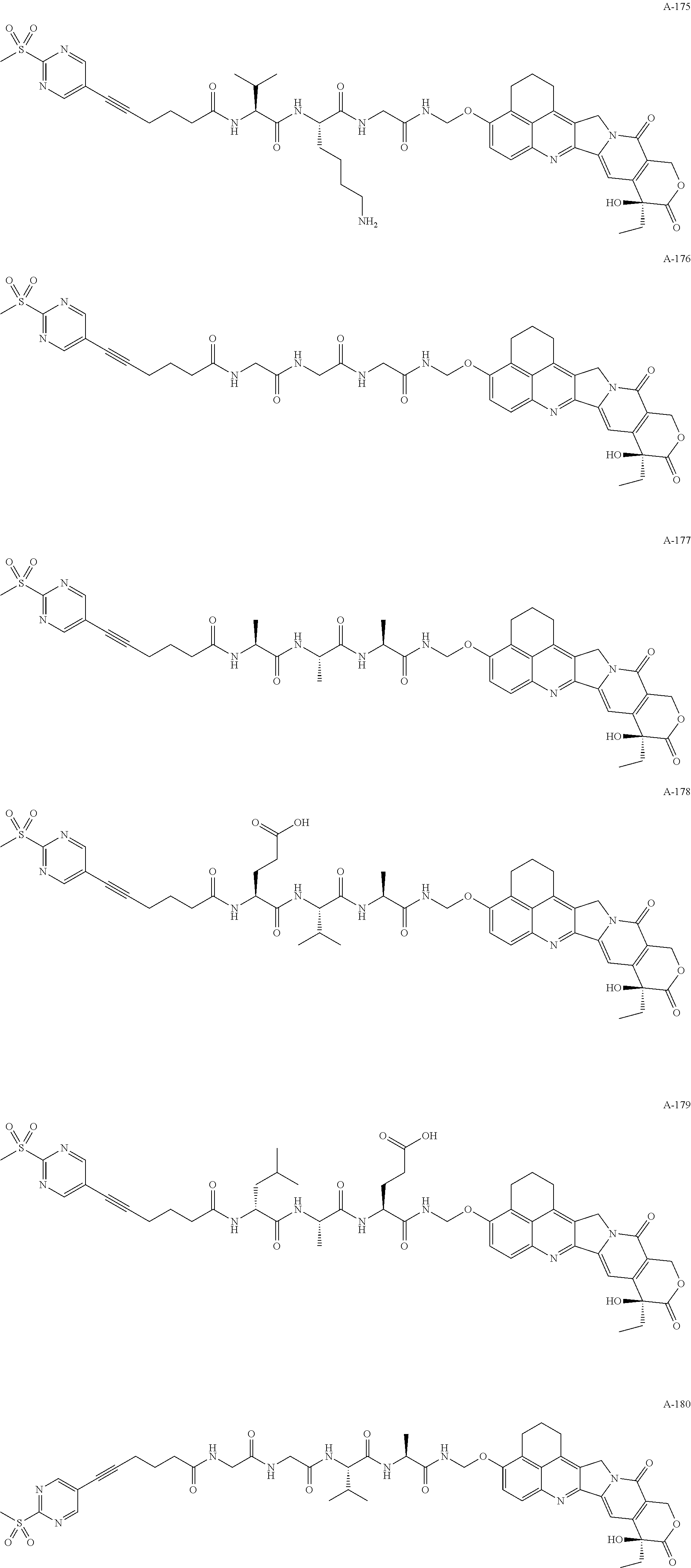 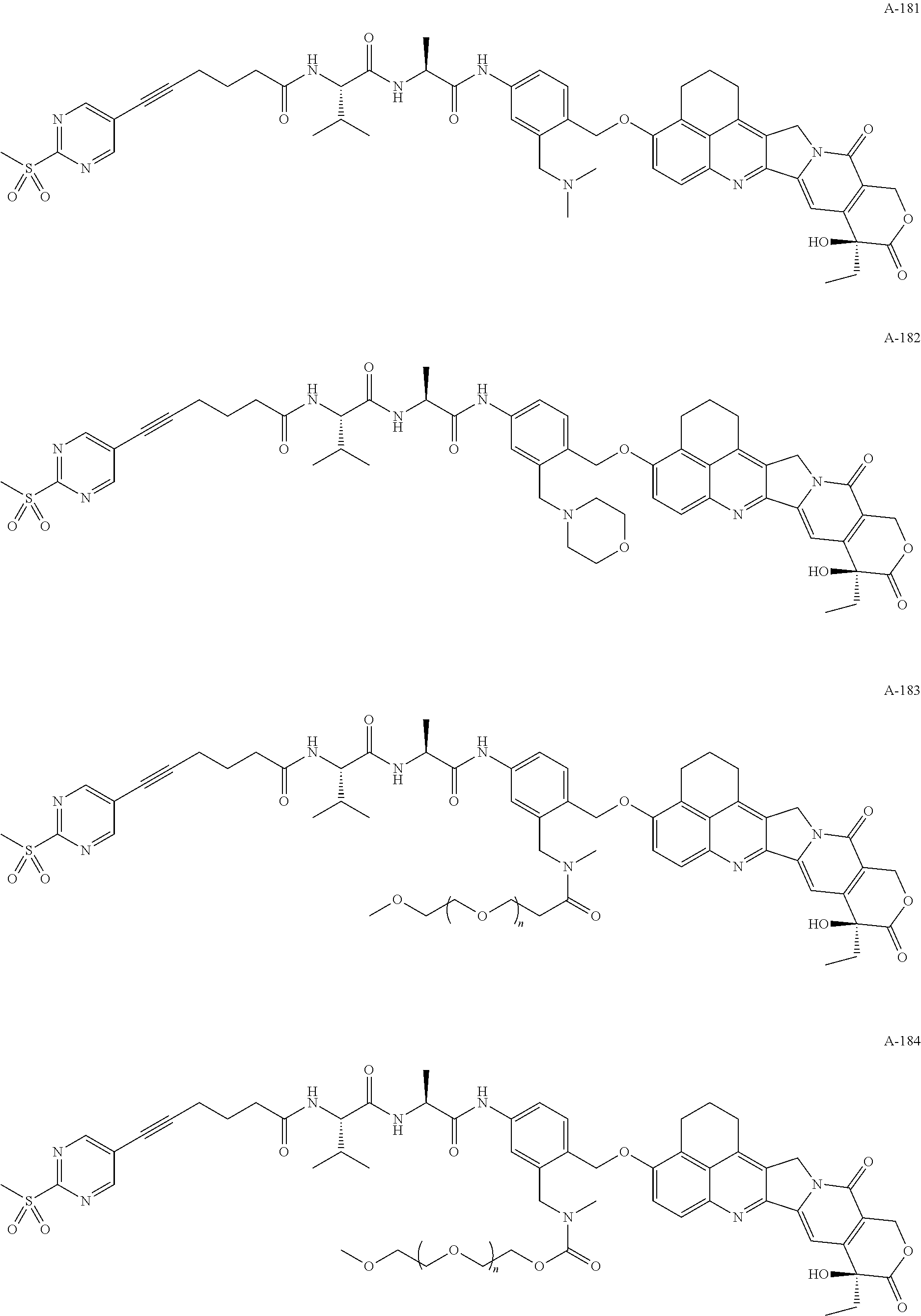 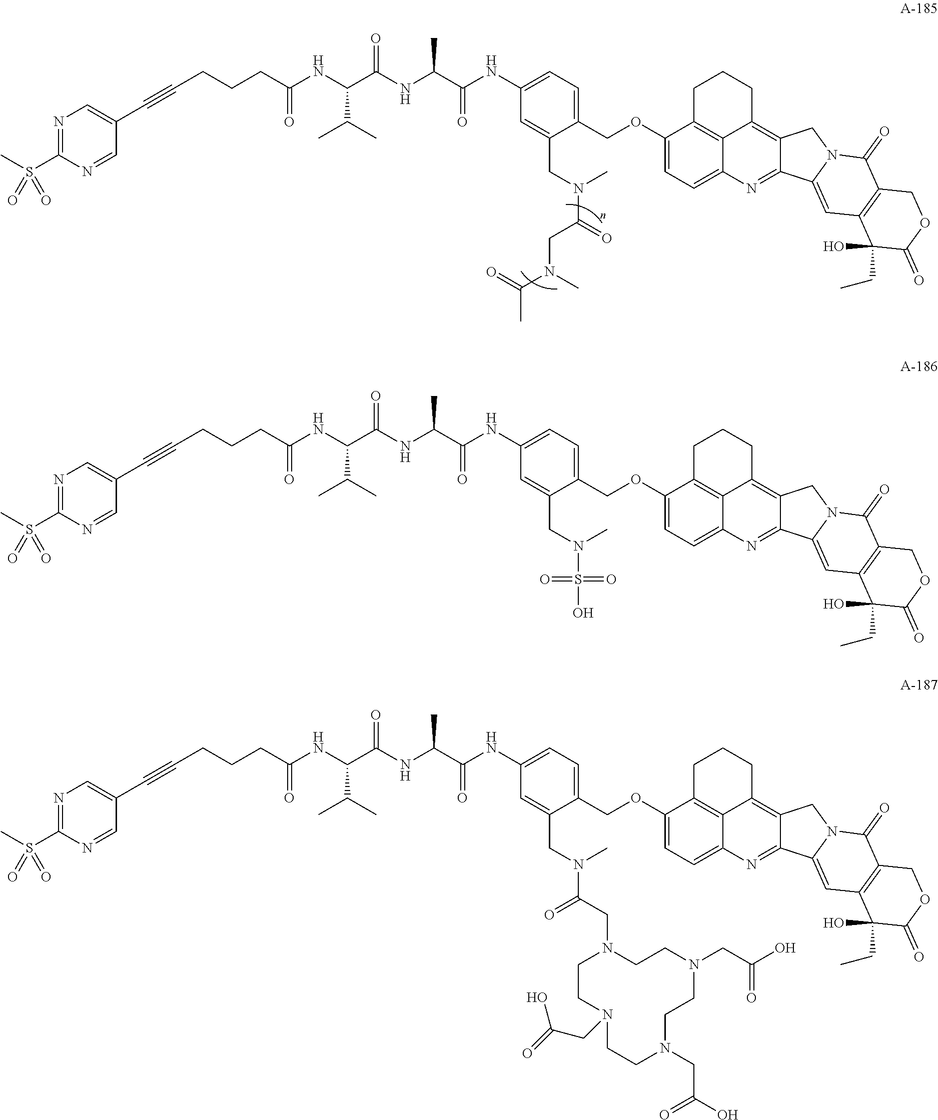 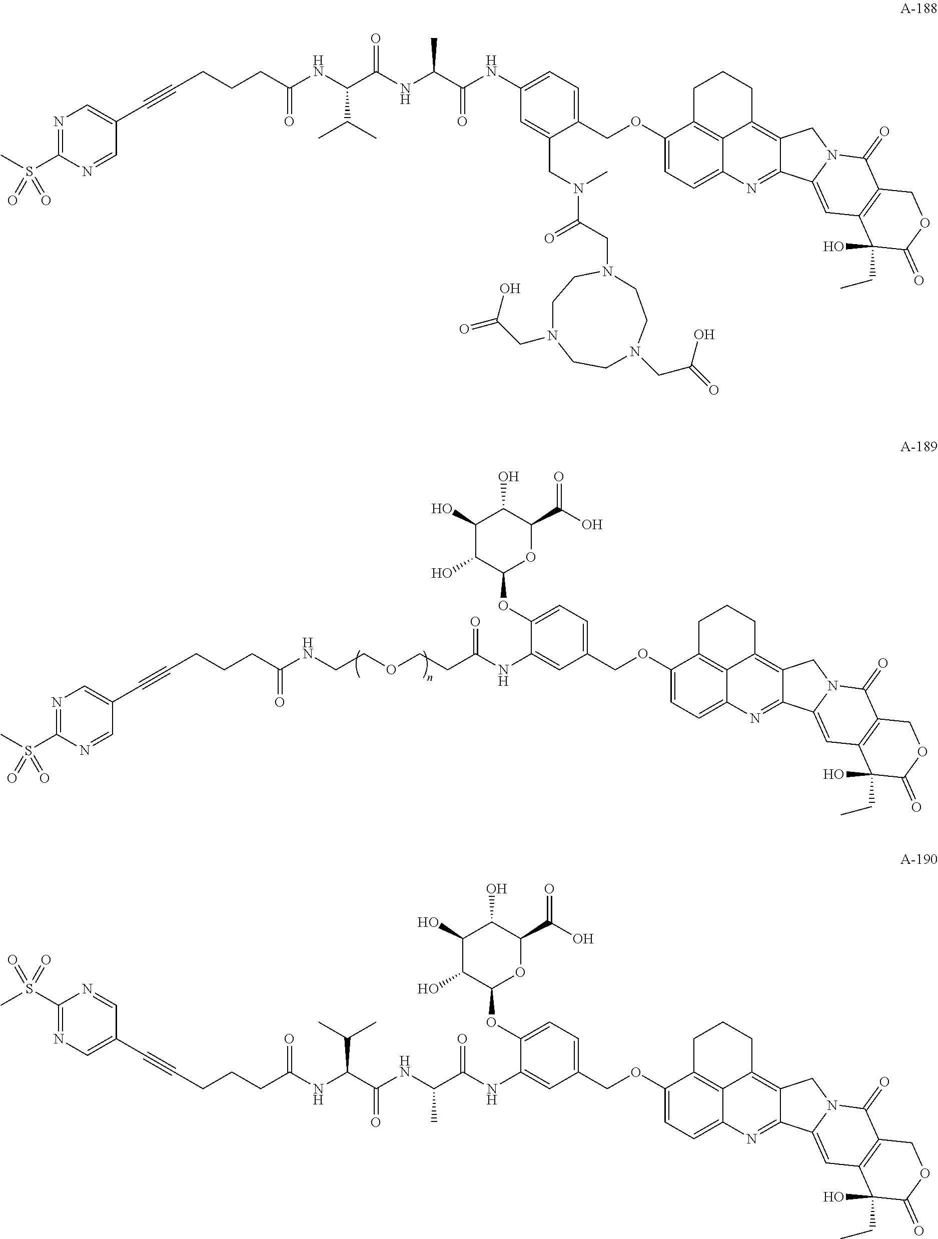 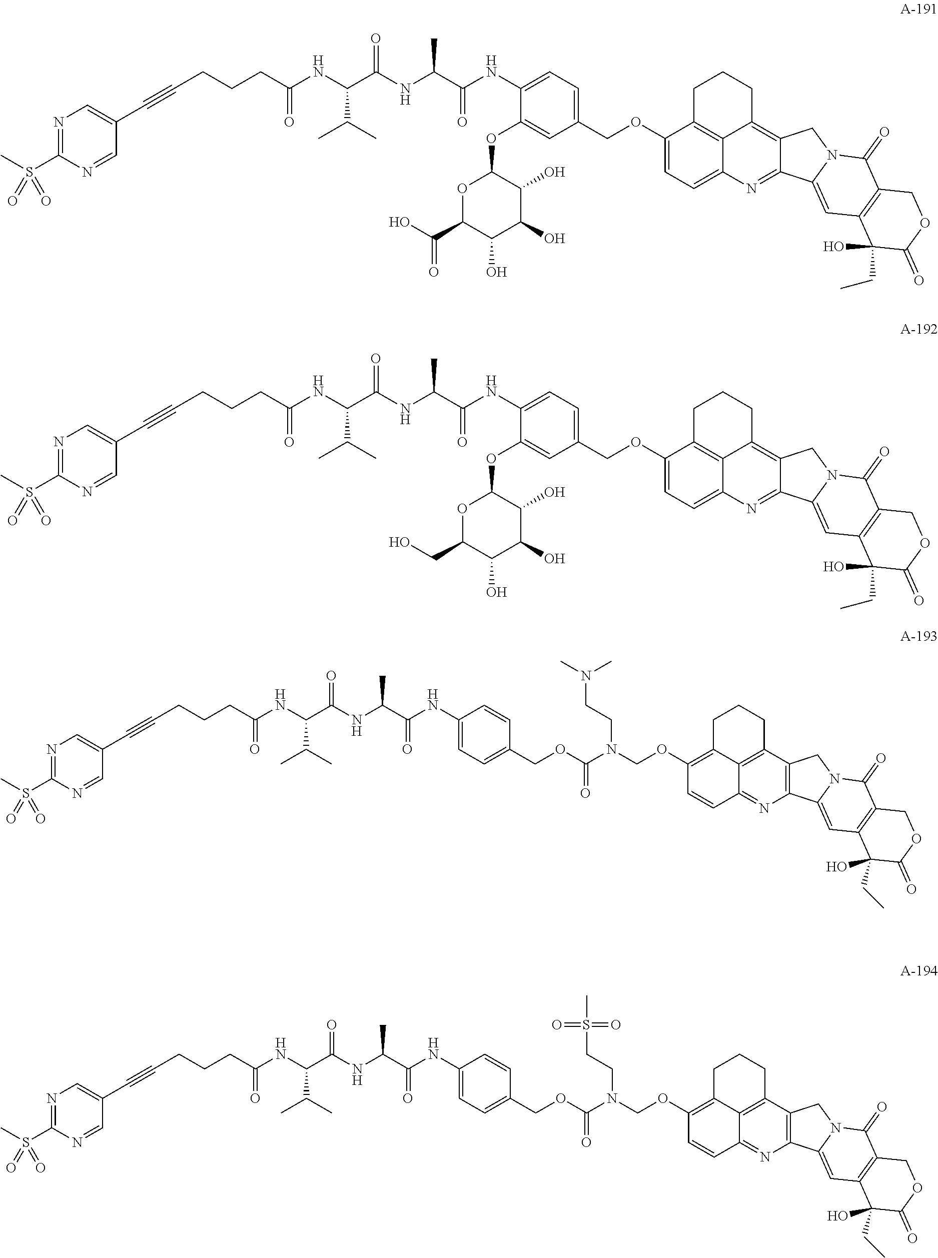 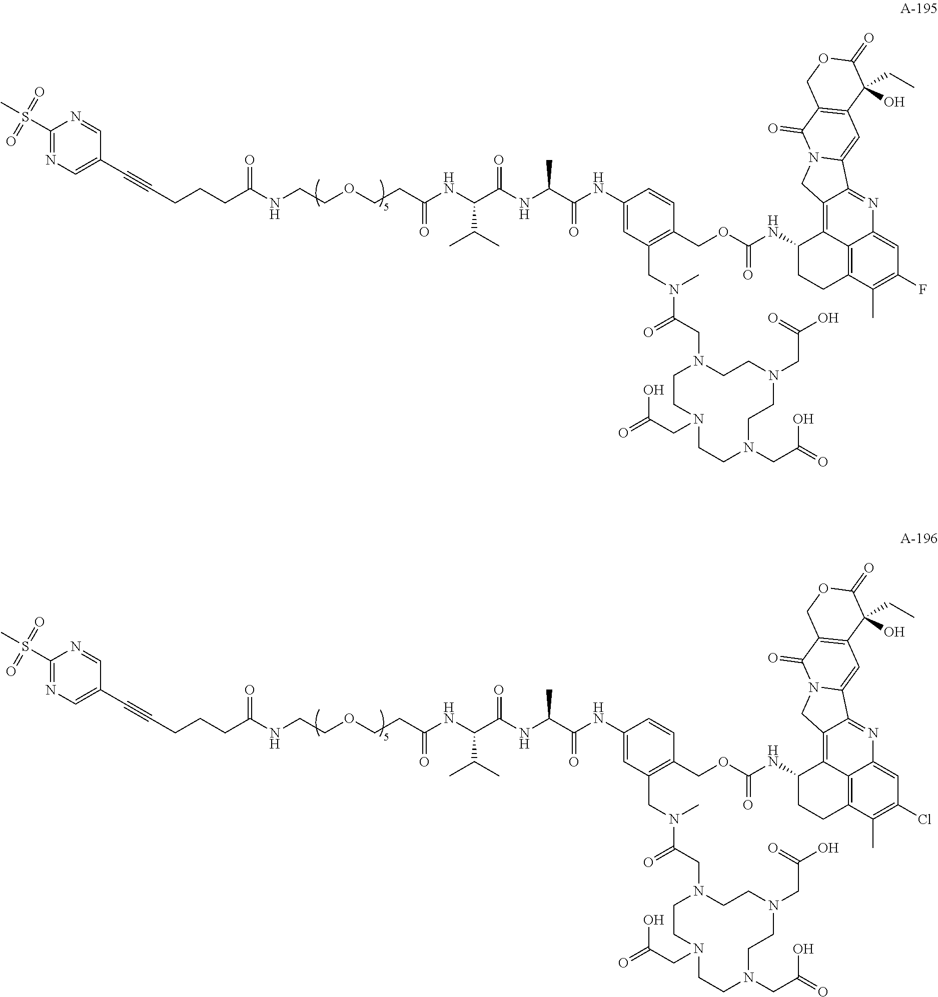 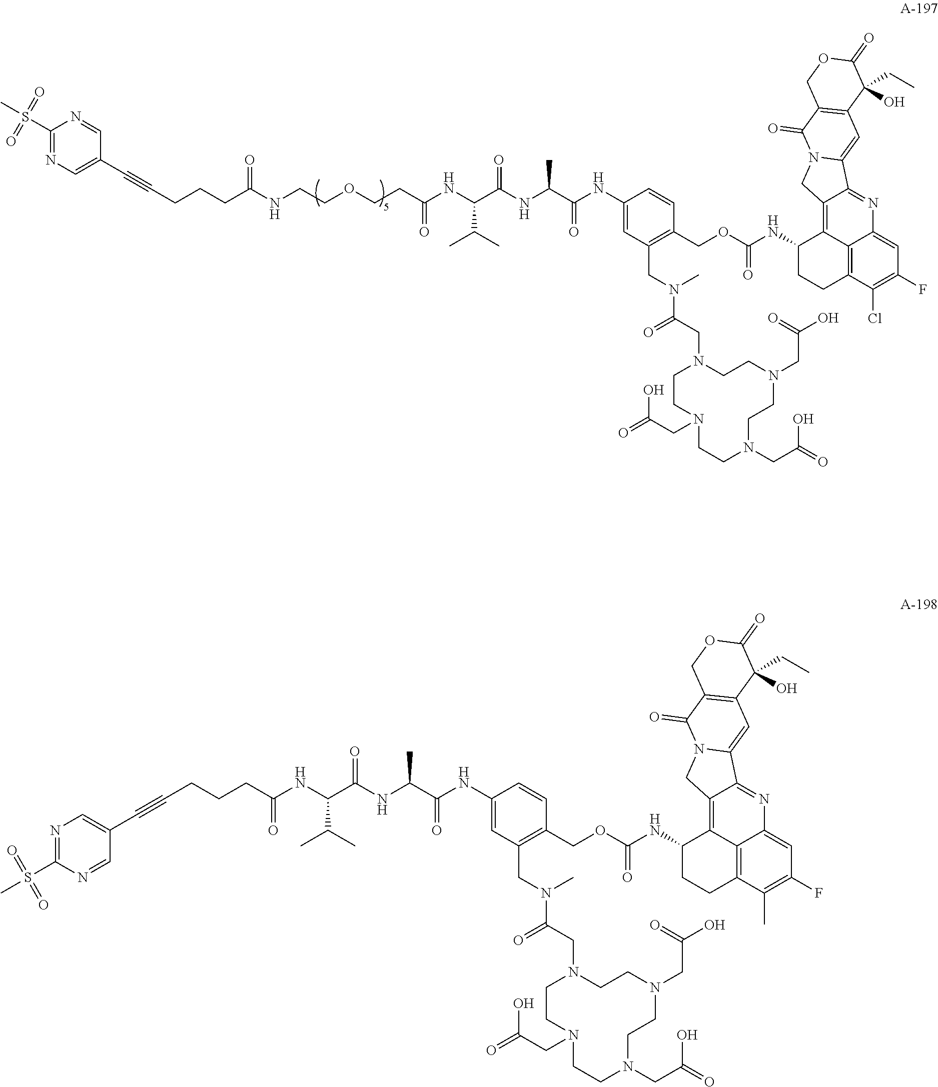 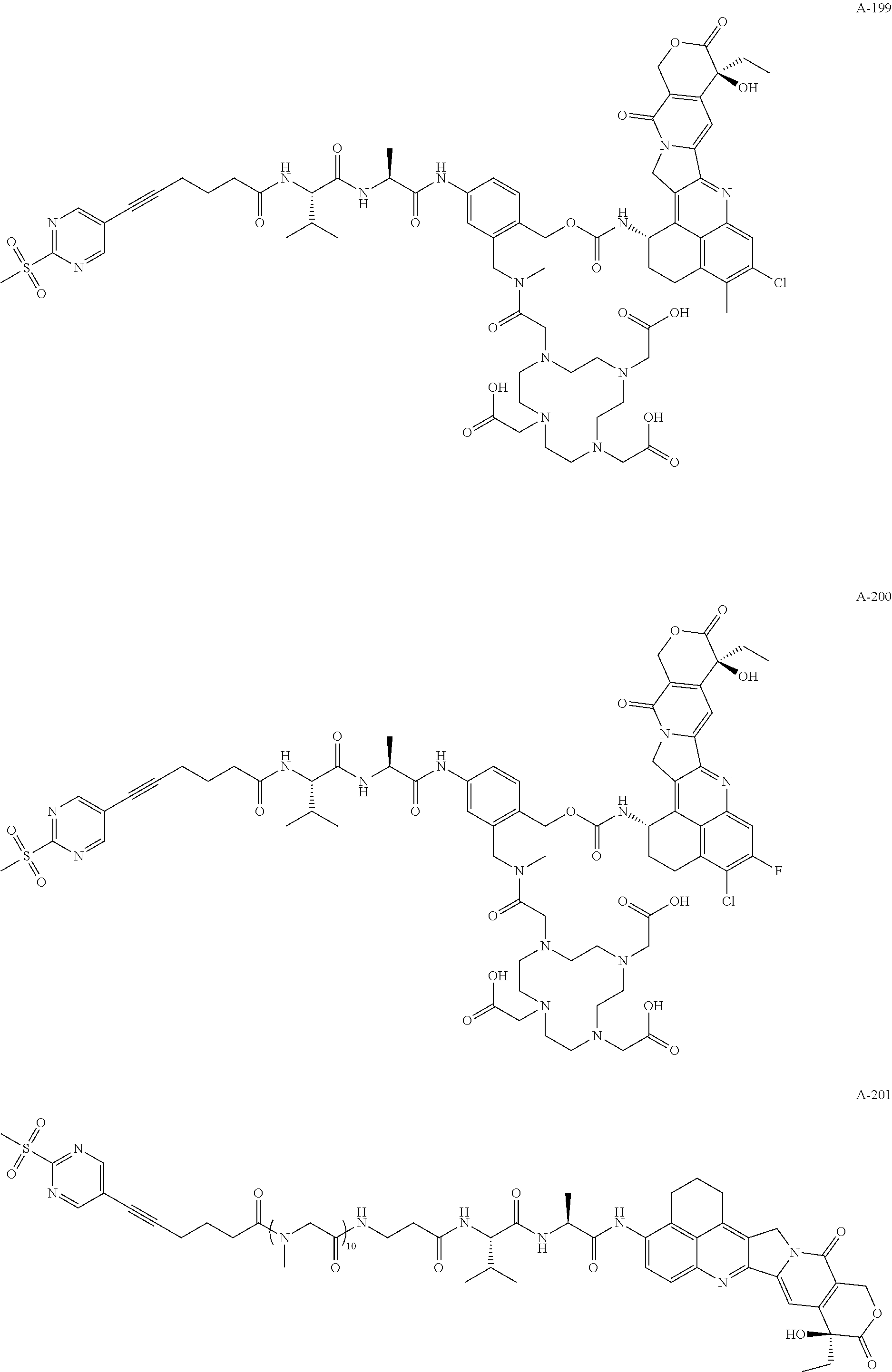 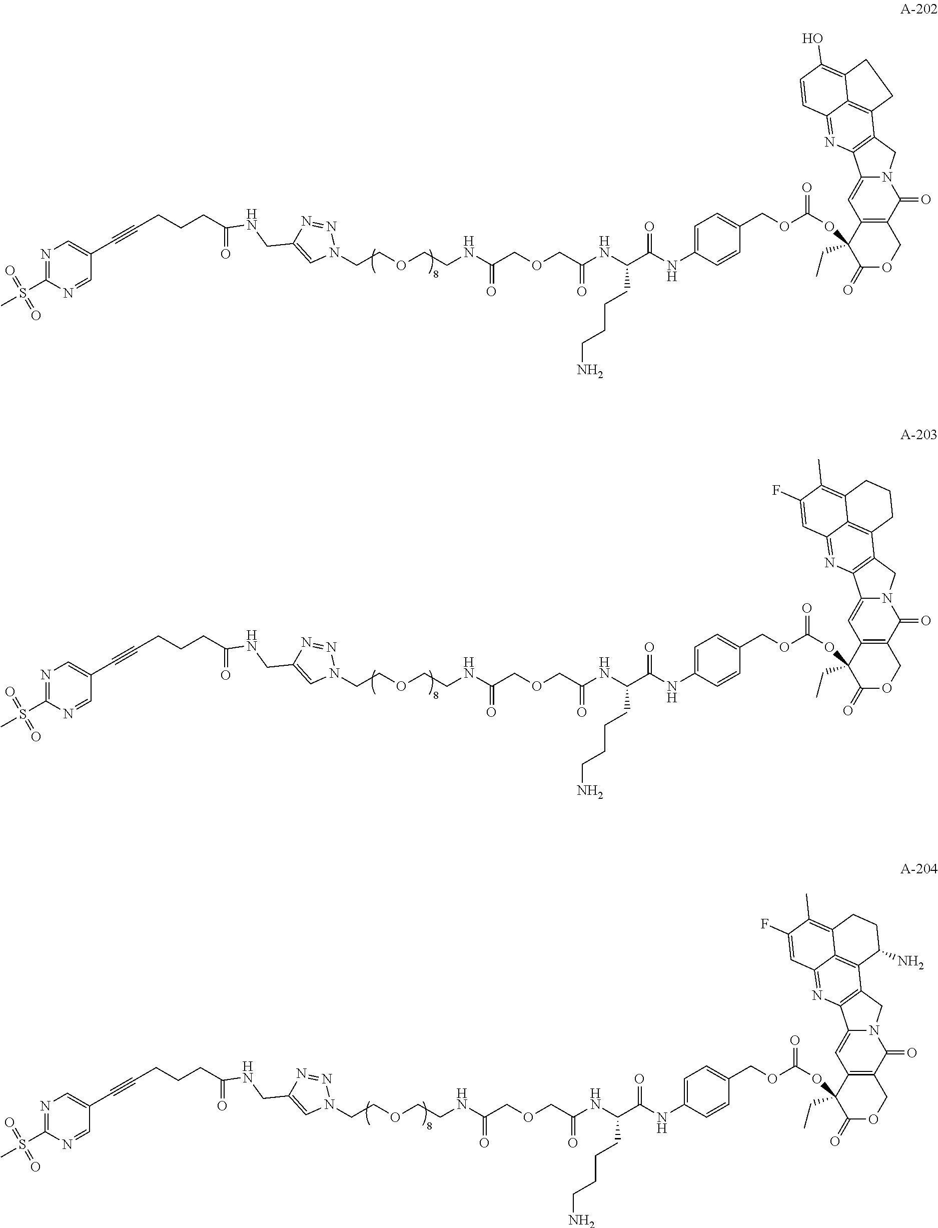 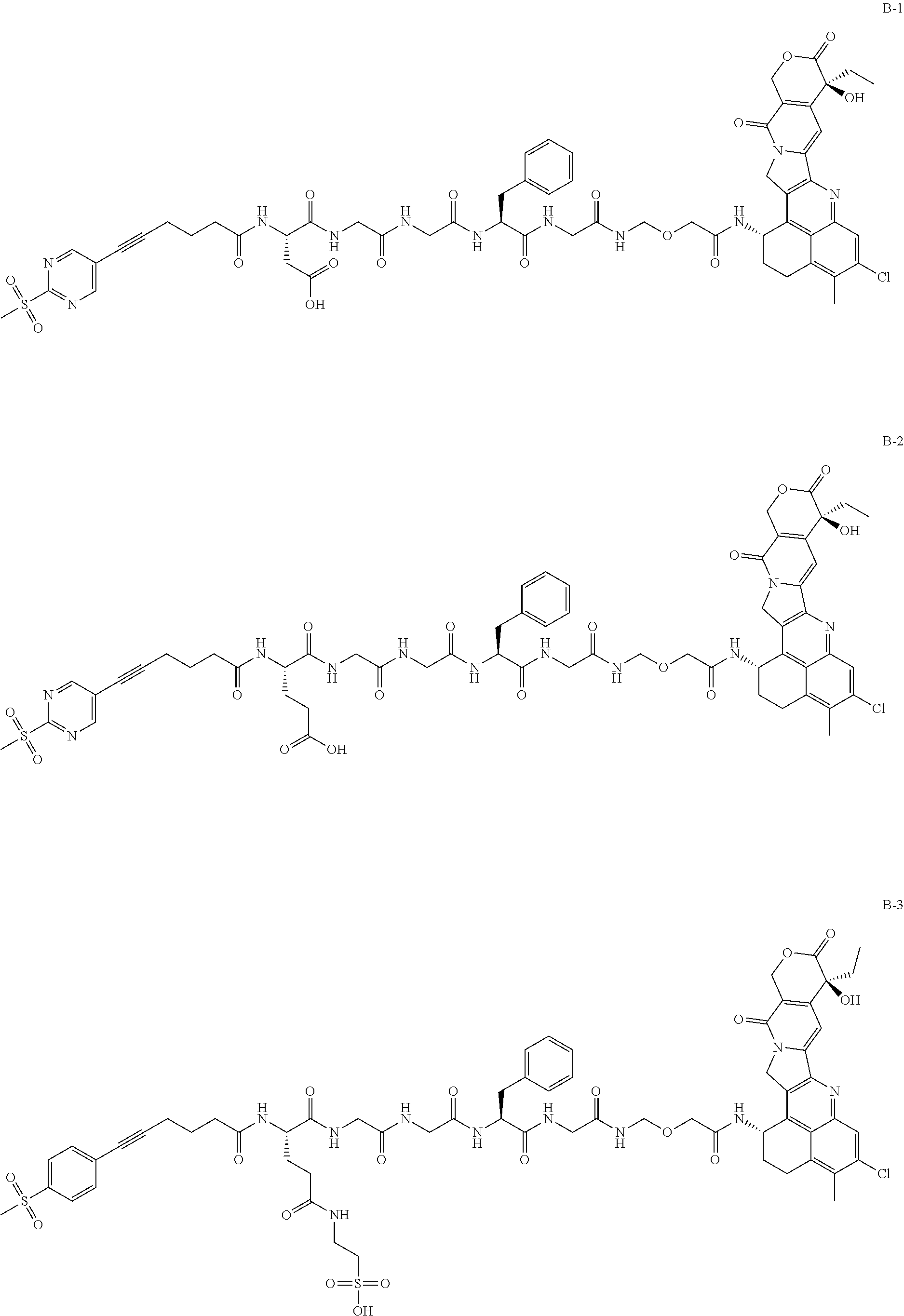 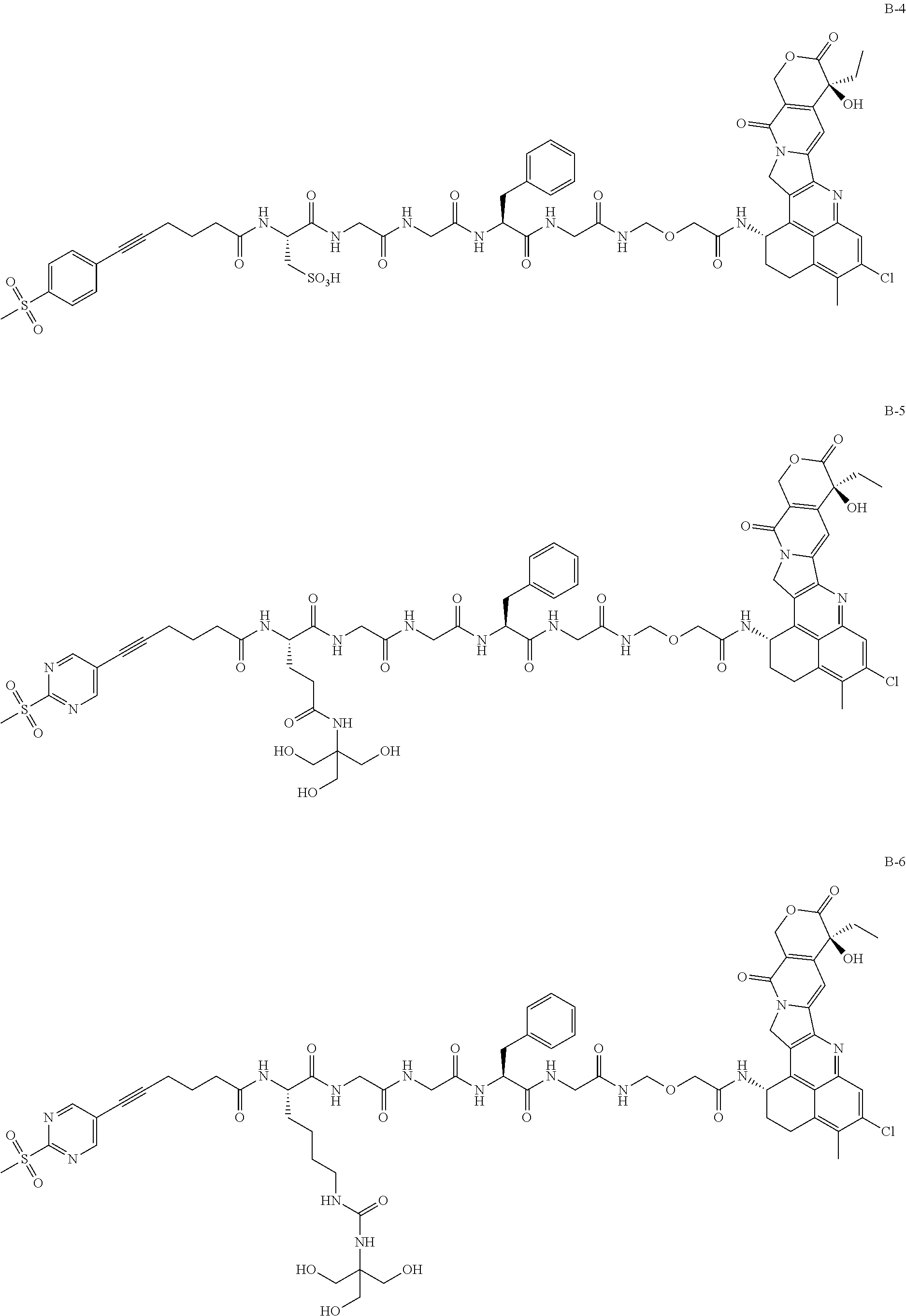 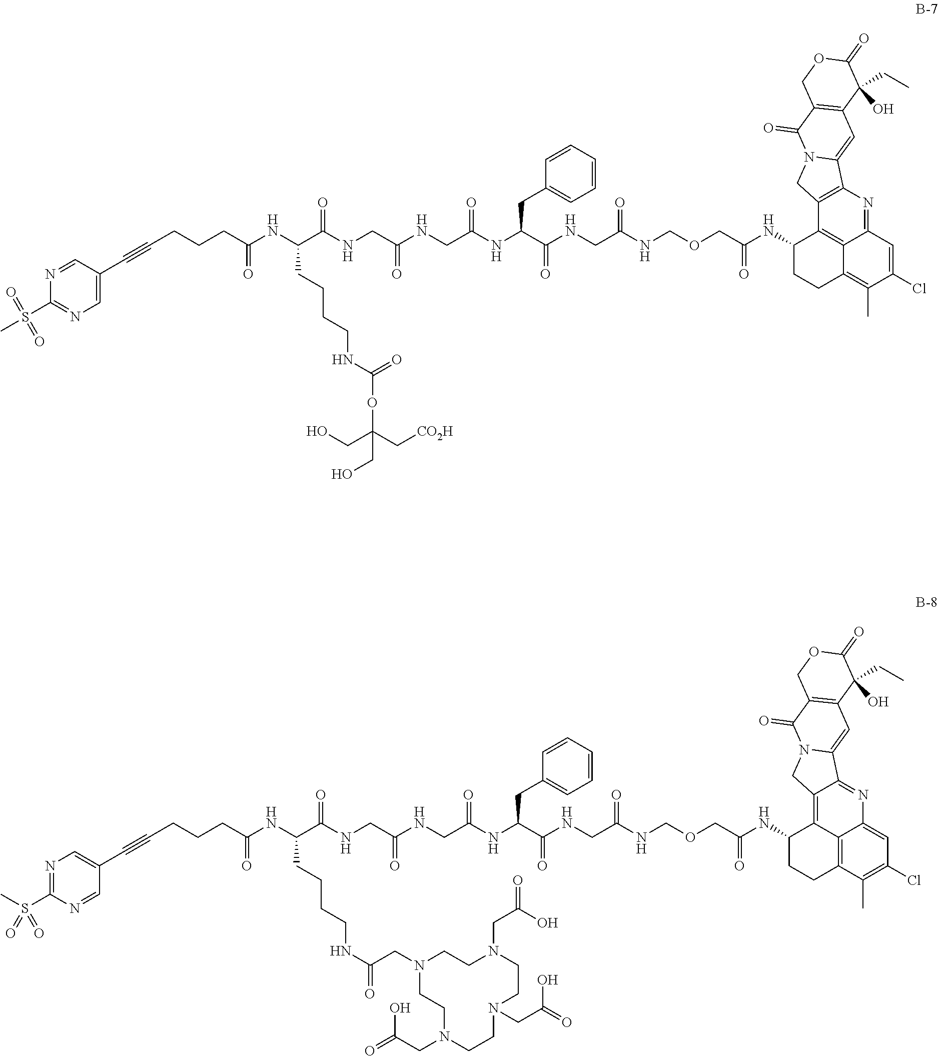 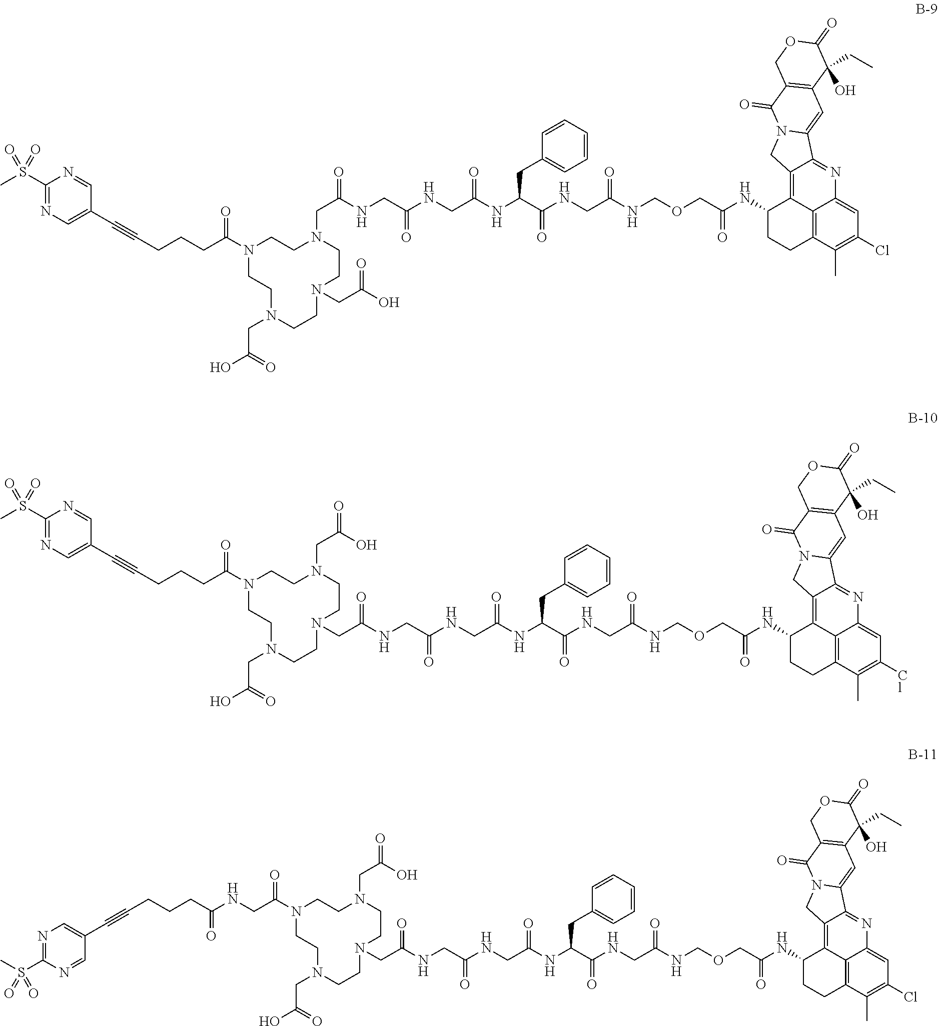 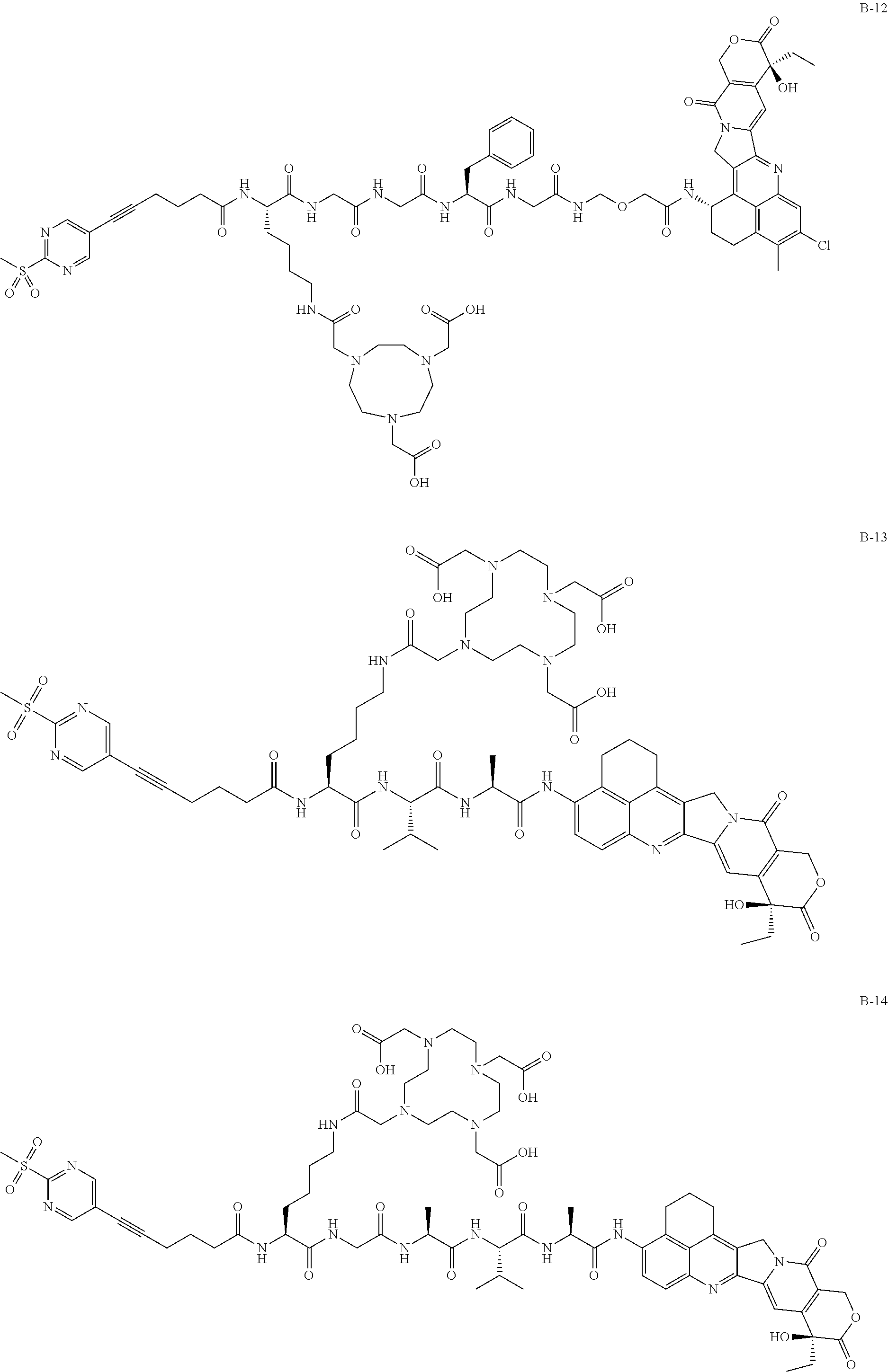 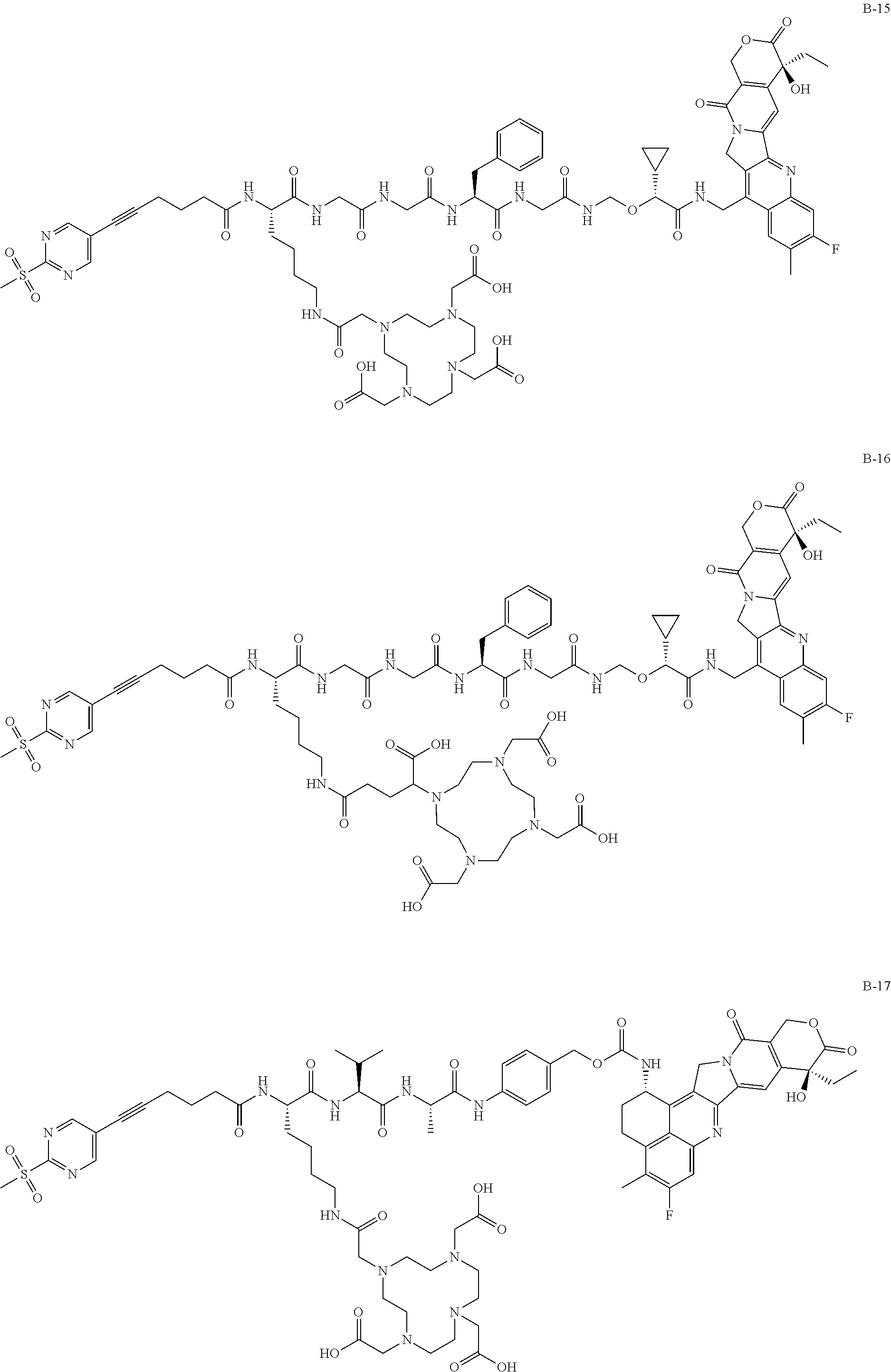 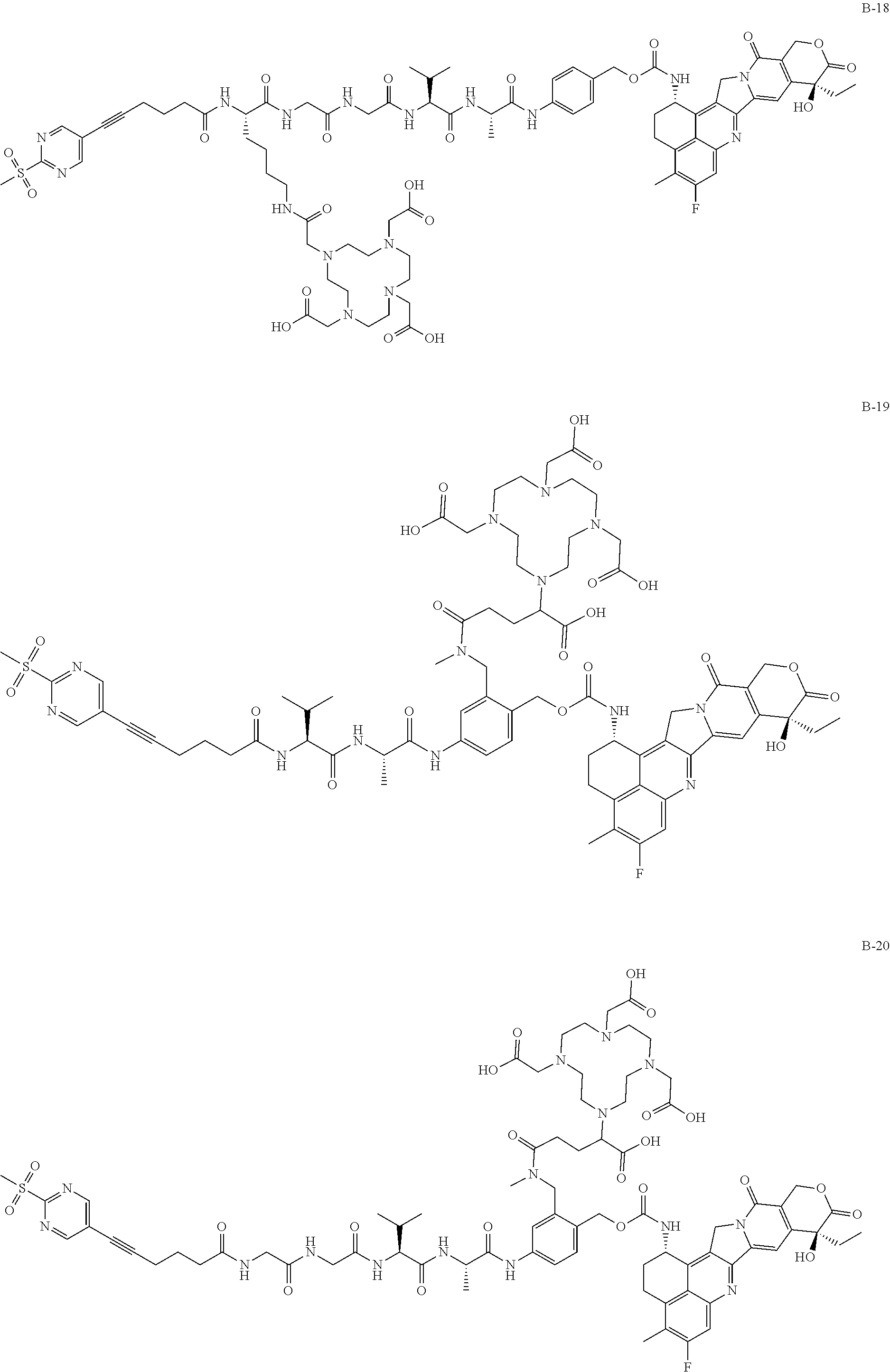 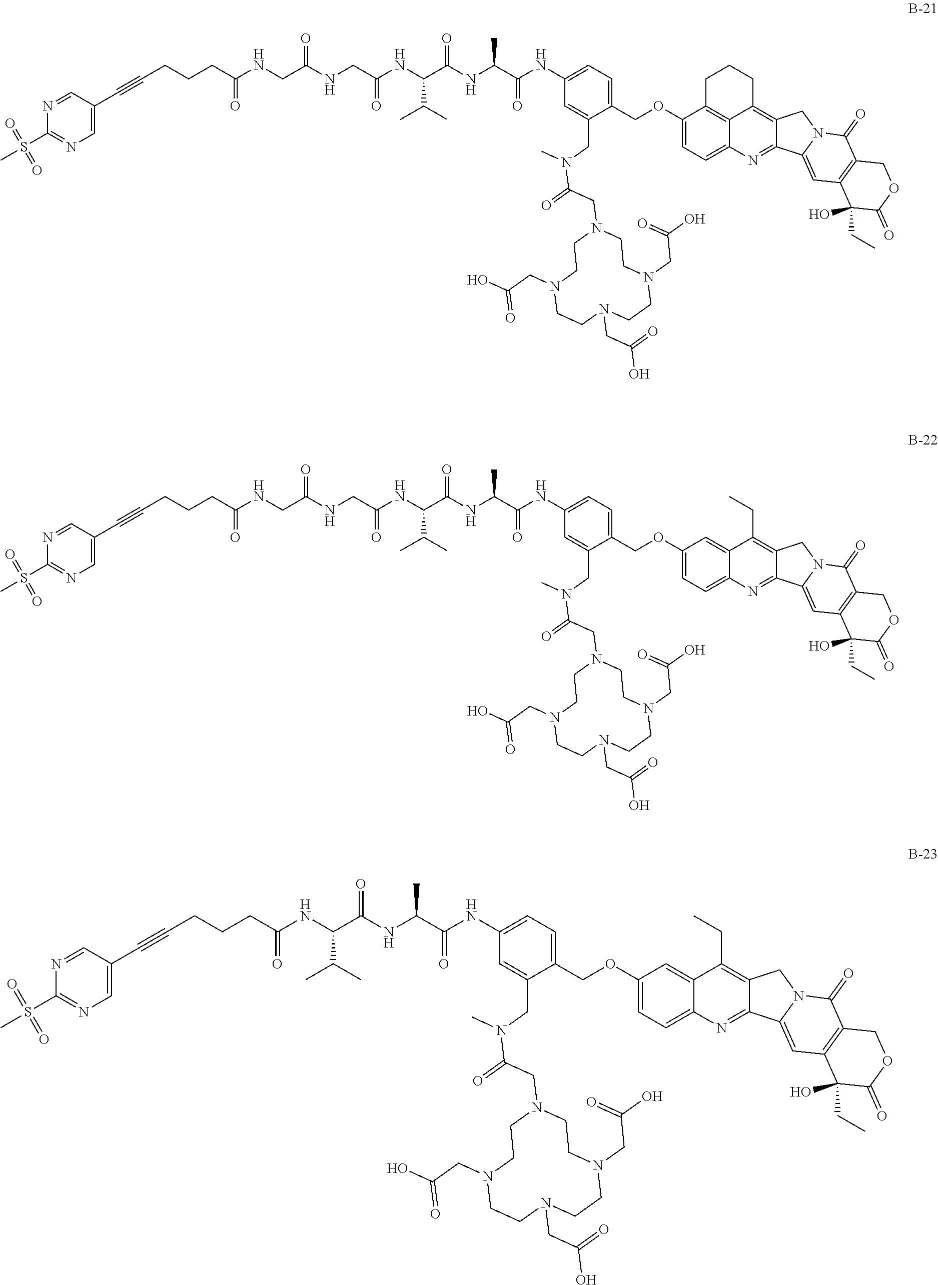 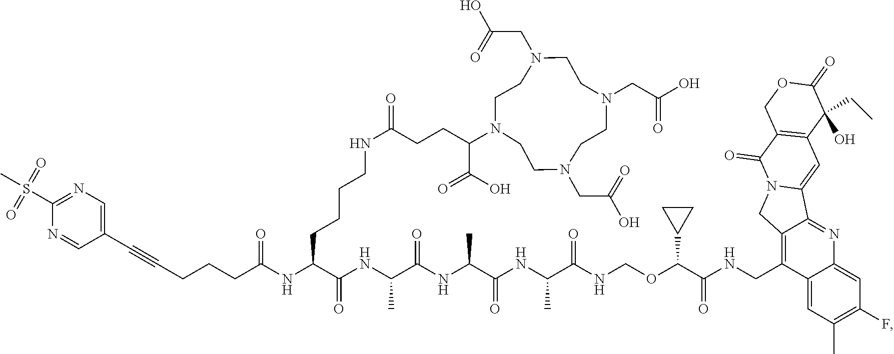
wherein, n is an integer selected from 1 to 20, such as integers of 1 to 12, 3 to 12, 5 to 15, 5 to 10, such as 1, 2, 3, 4, 5, 6, 7, 8, 9, 10, 11, 12, 13, 14, 15, 16, 17, 18, 19, or 20.

8. A conjugate represented by Formula (II), wherein: 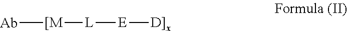
Ab represents a targeting moiety; M, L, E and D are as described in claim 1;
x is Ito 10.

9. The conjugate according to claim 8, wherein the Ab is an antibody or antigen-binding fragment thereof, preferably an antibody or antigen-binding fragment thereof capable of specifically binding to epidermal growth factor receptor 2 (Her2), a member of the ErbB family of receptor tyrosine kinases.

10. The conjugate according to claim 8, which is selected from the group consisting of: 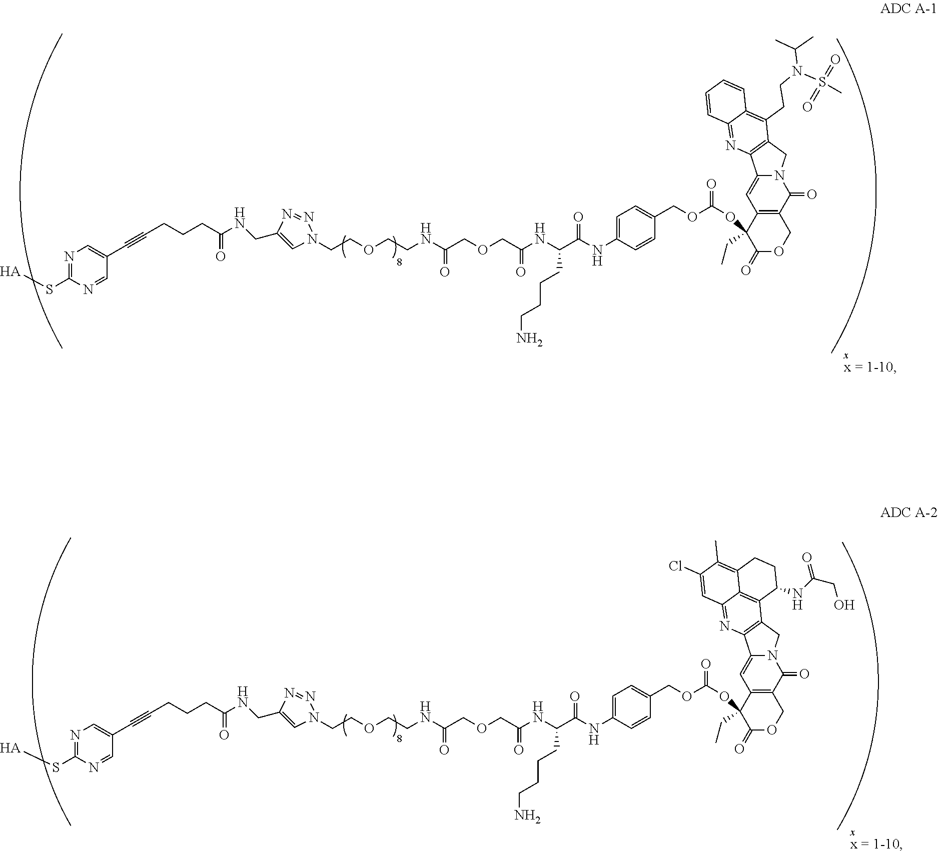 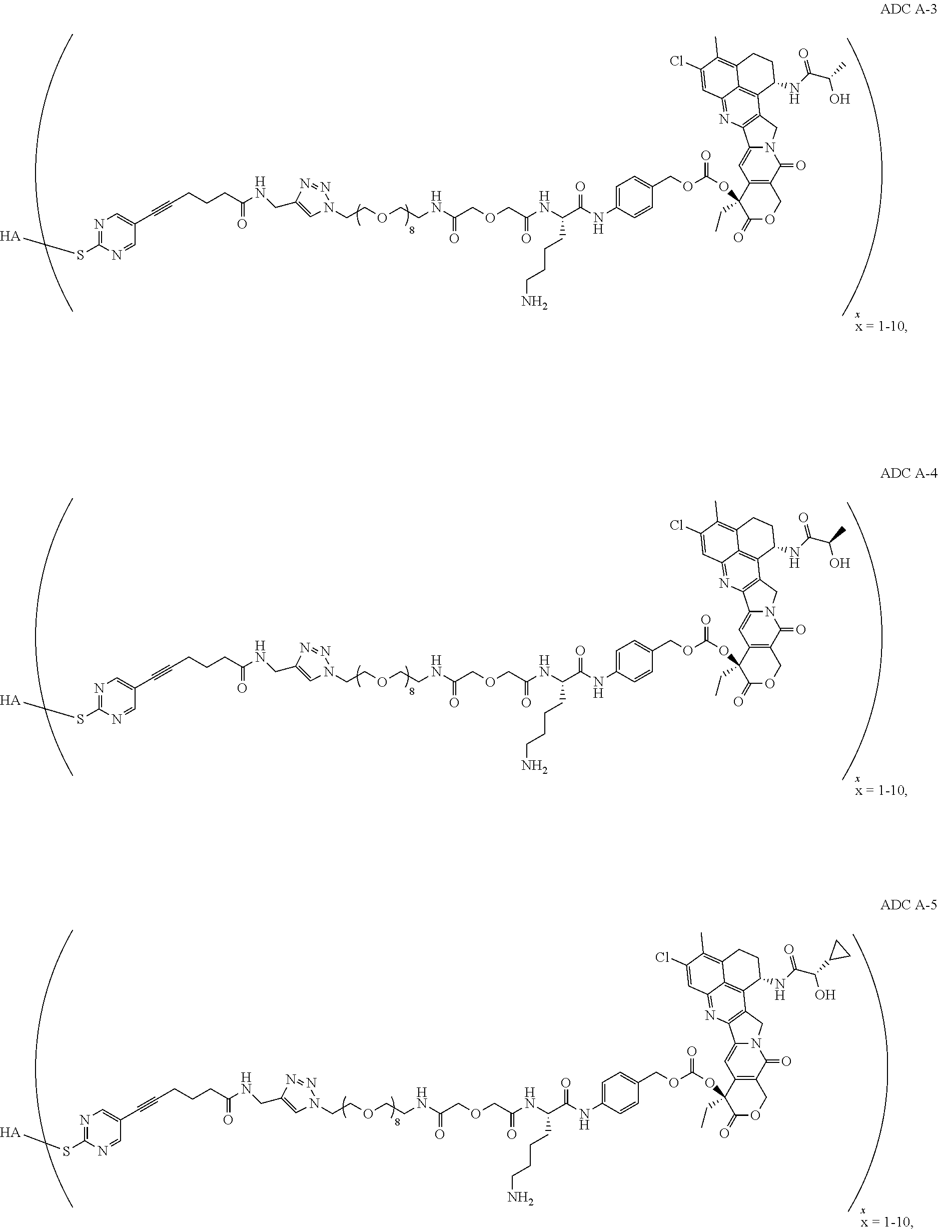 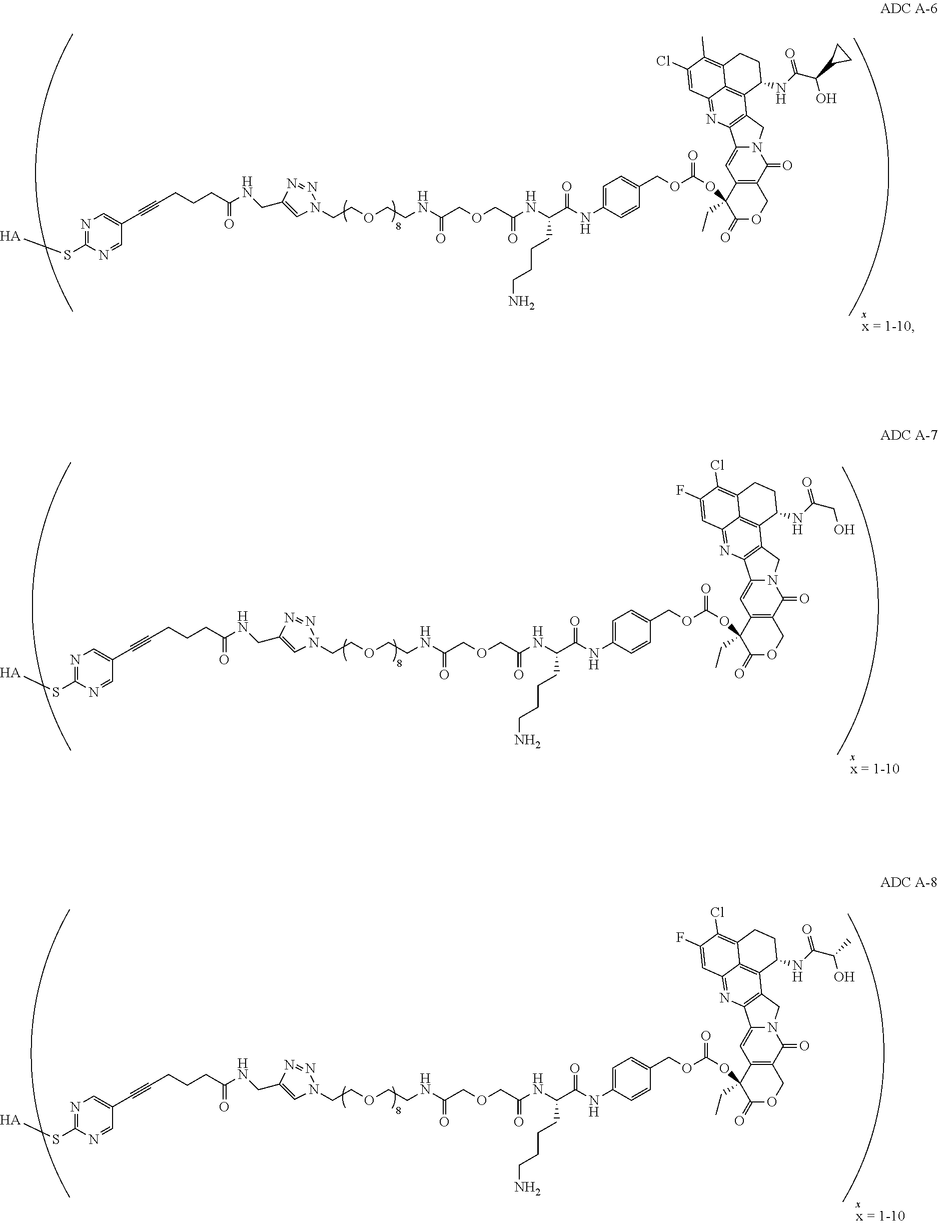 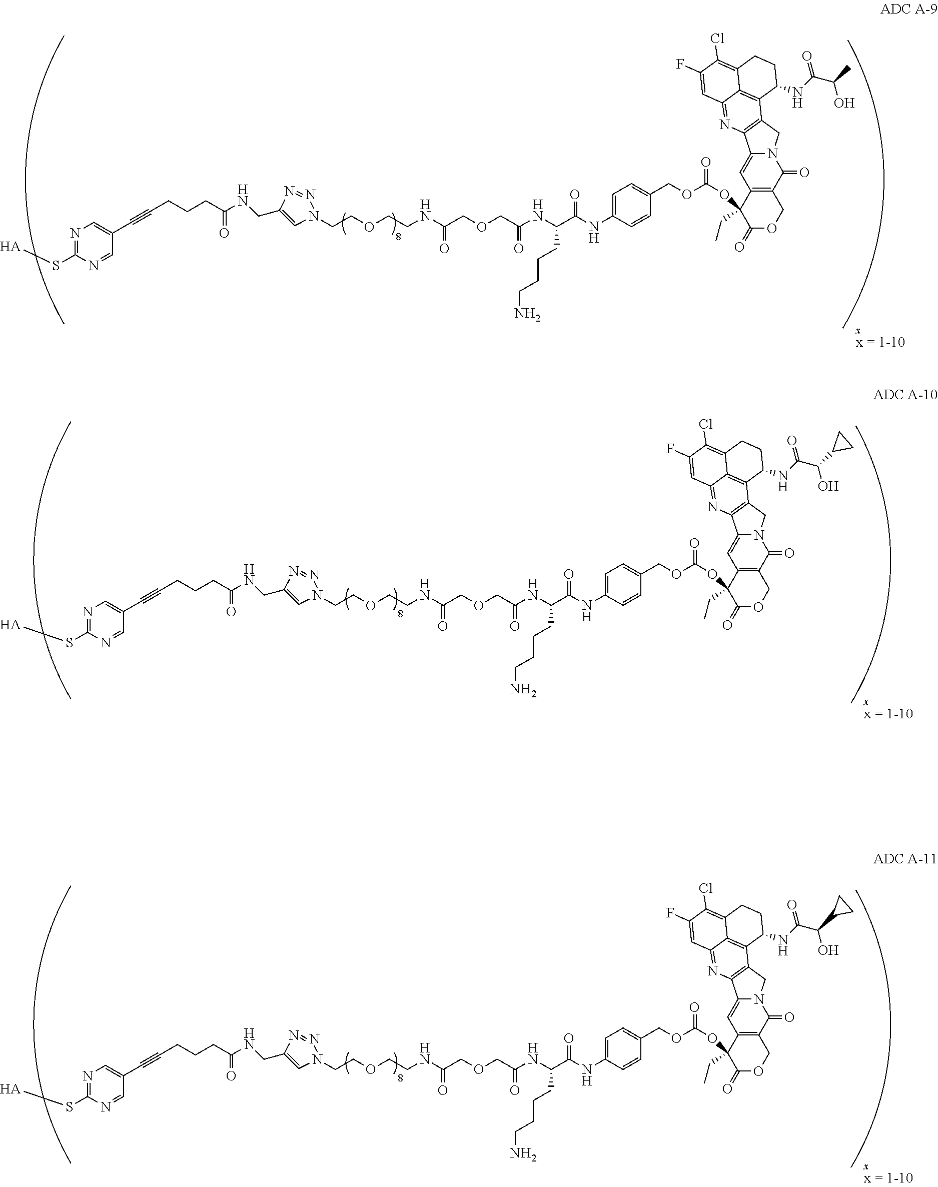 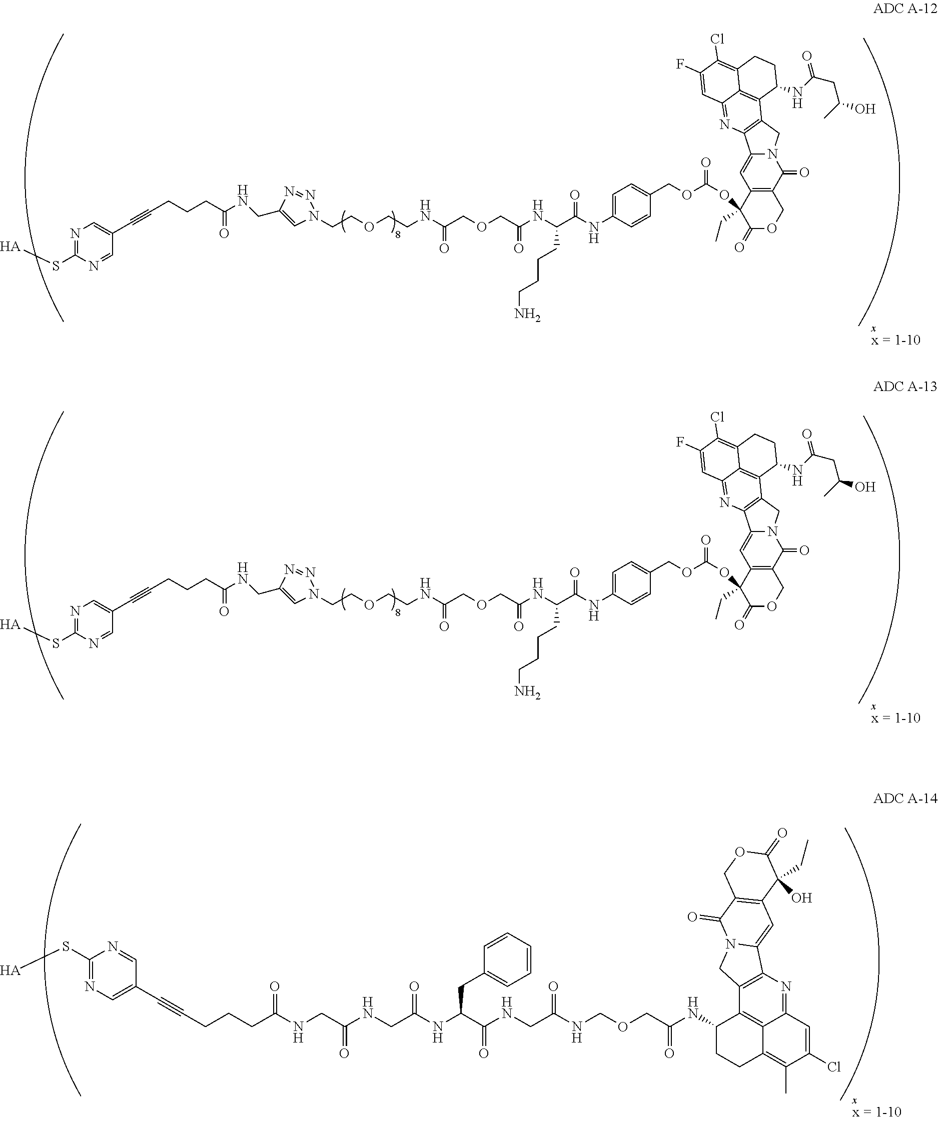 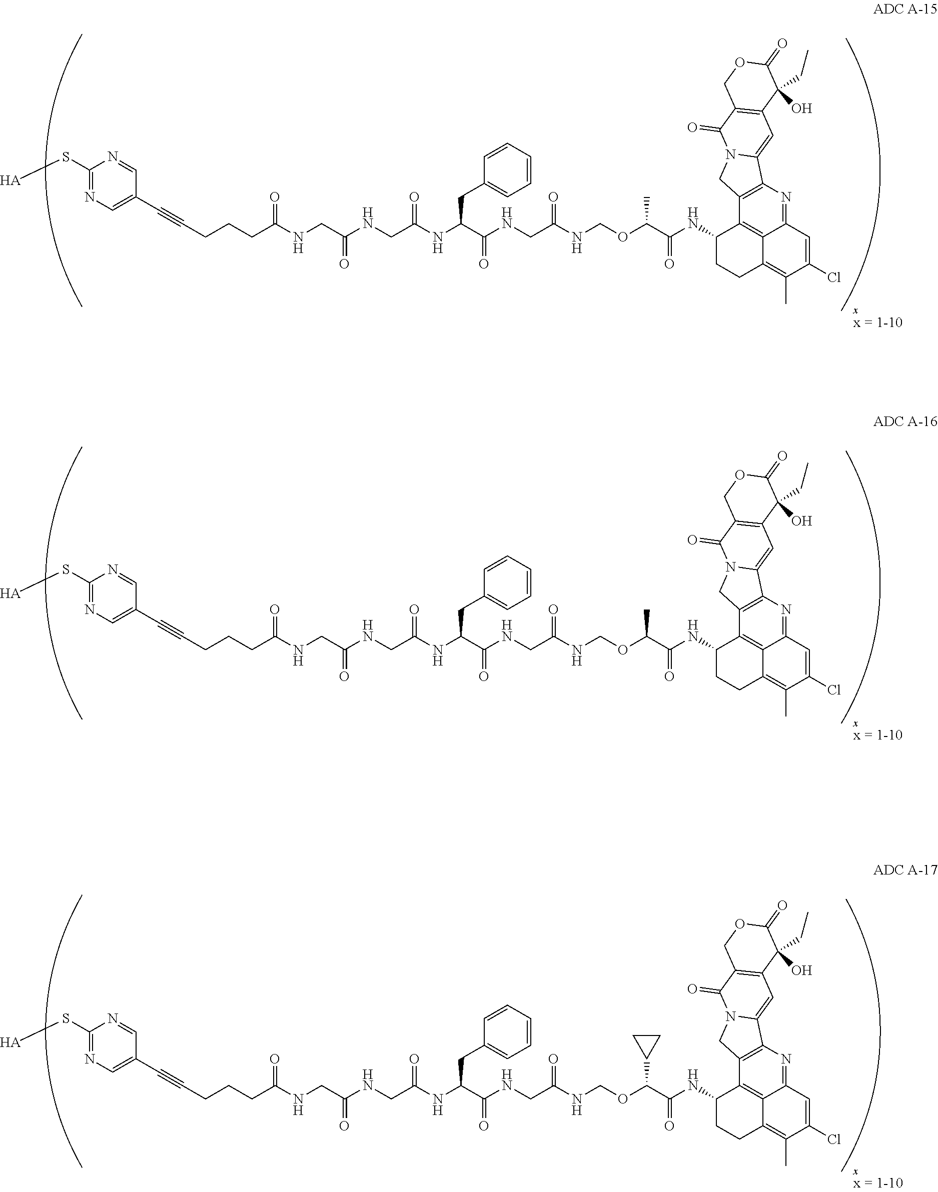 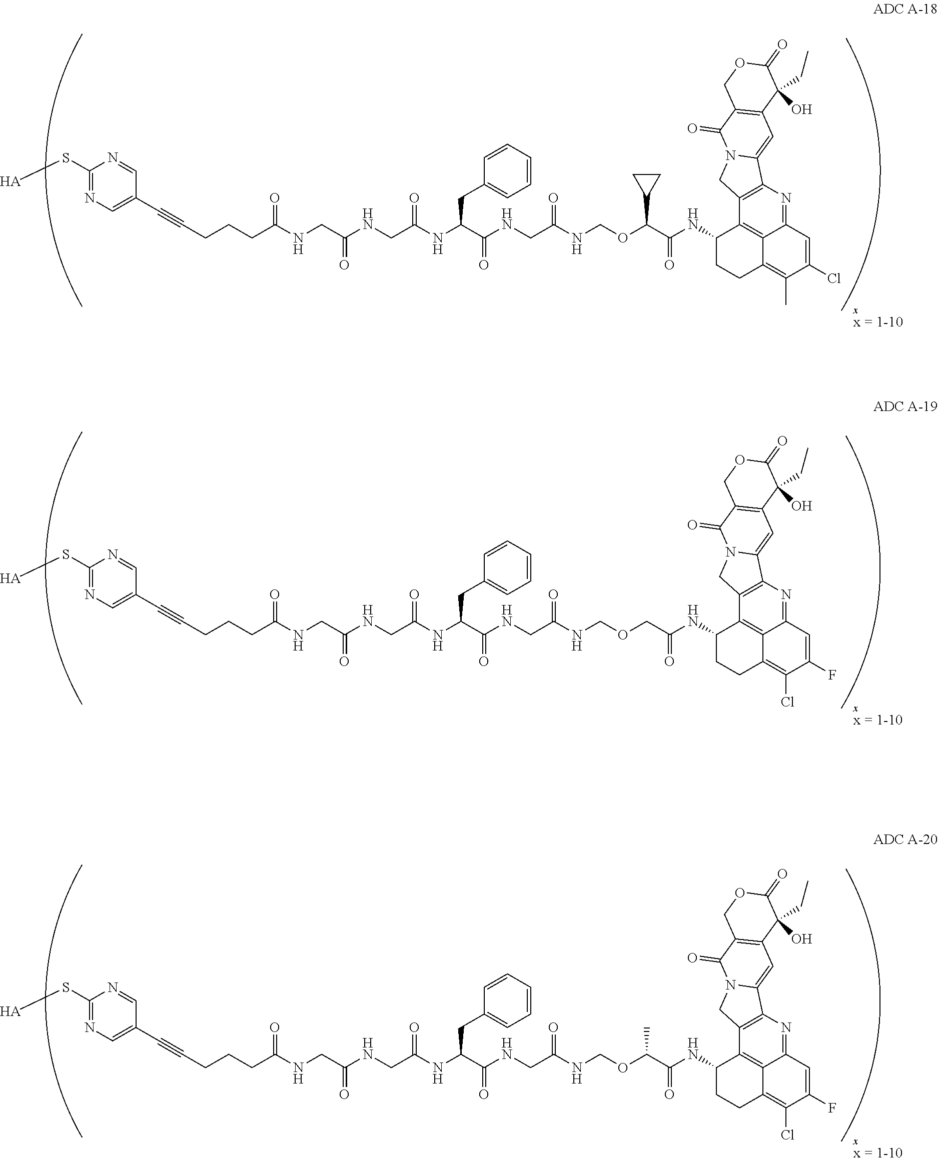 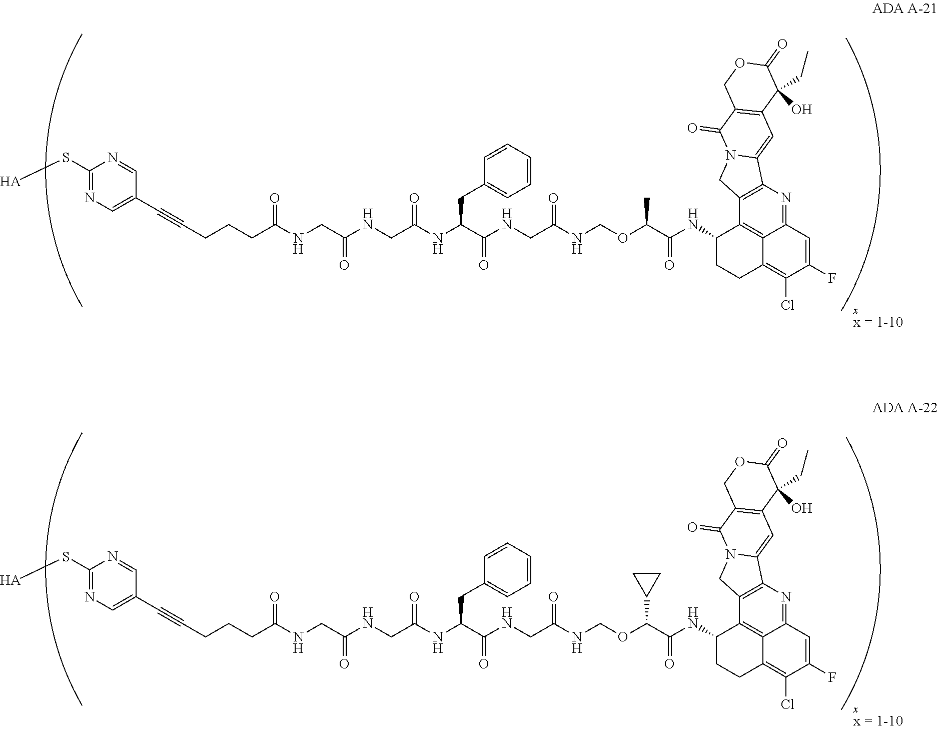 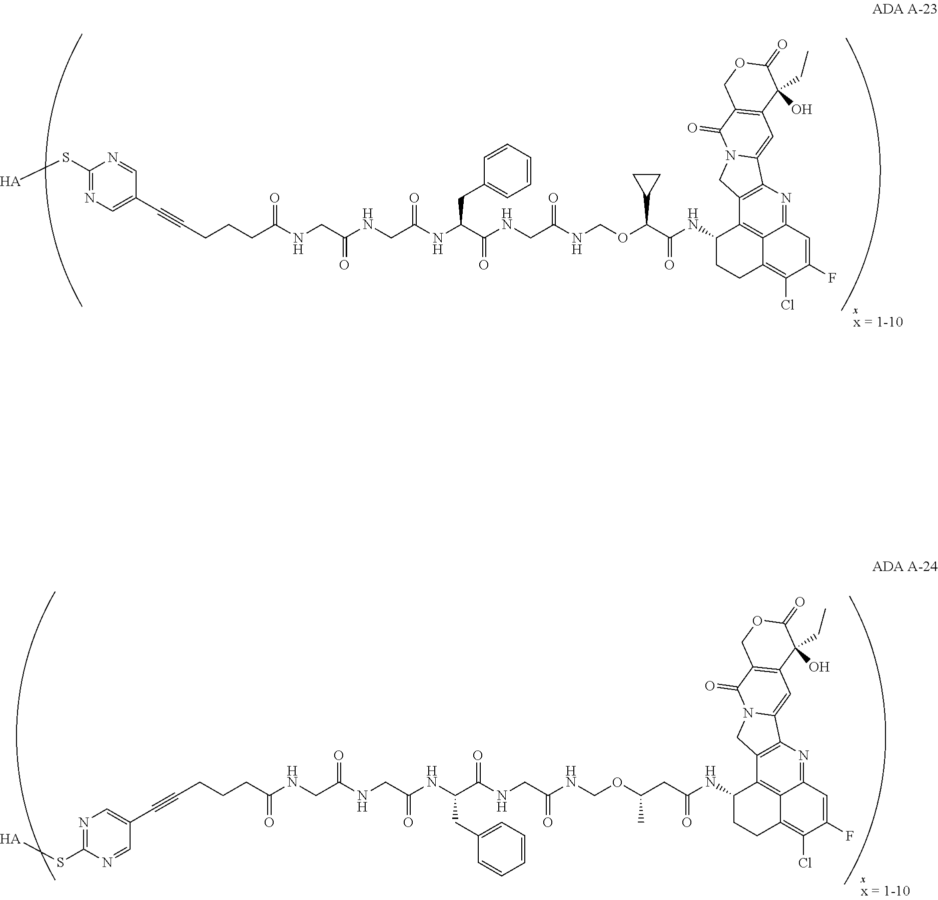 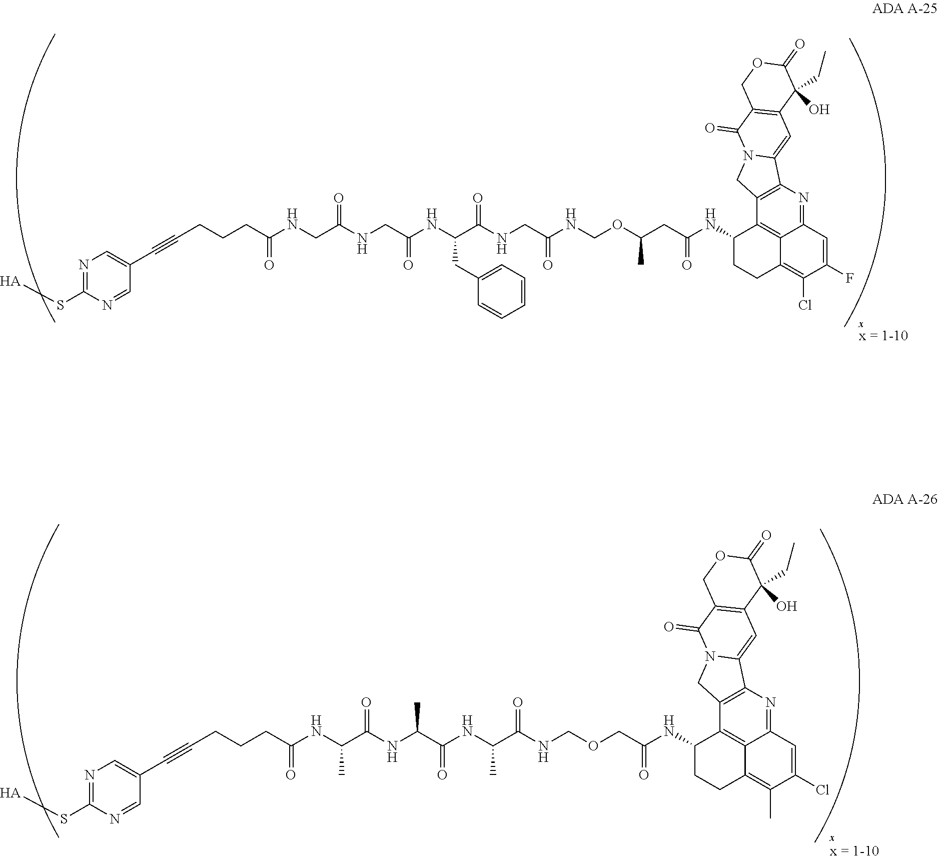 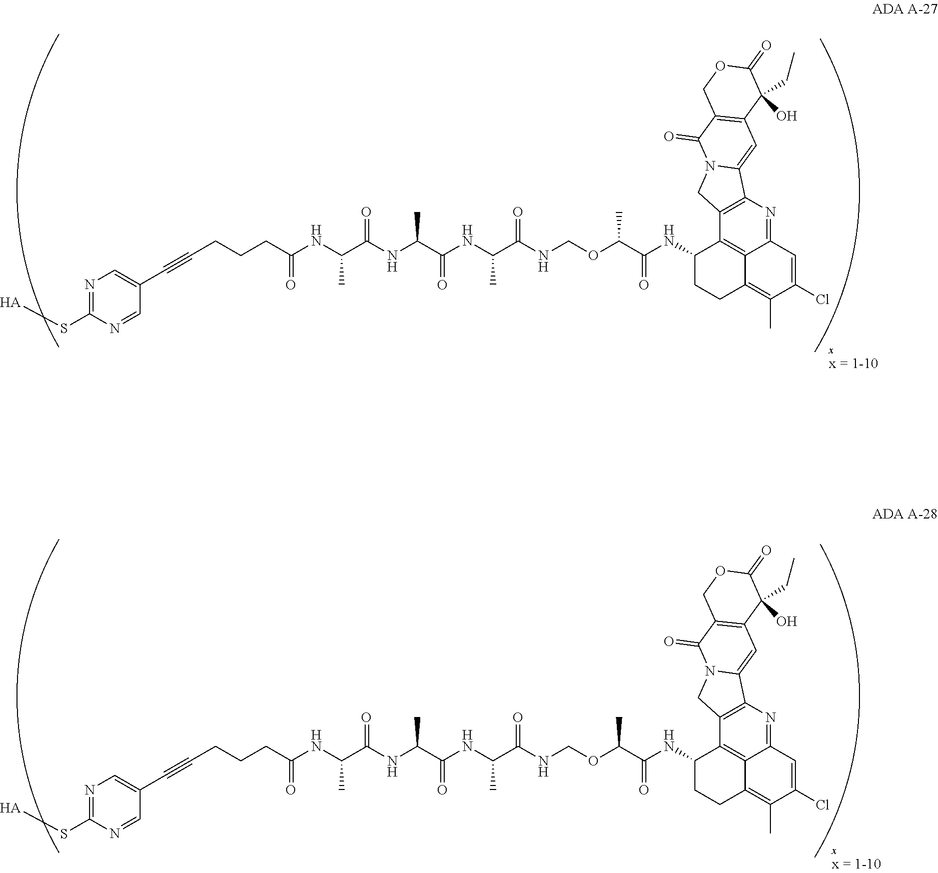 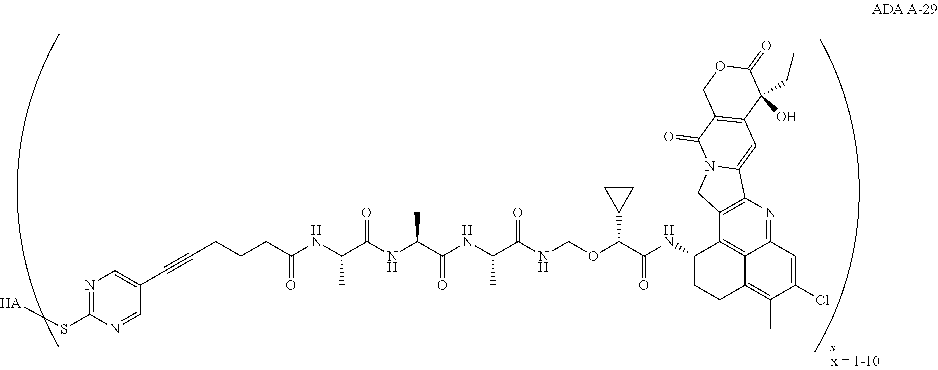 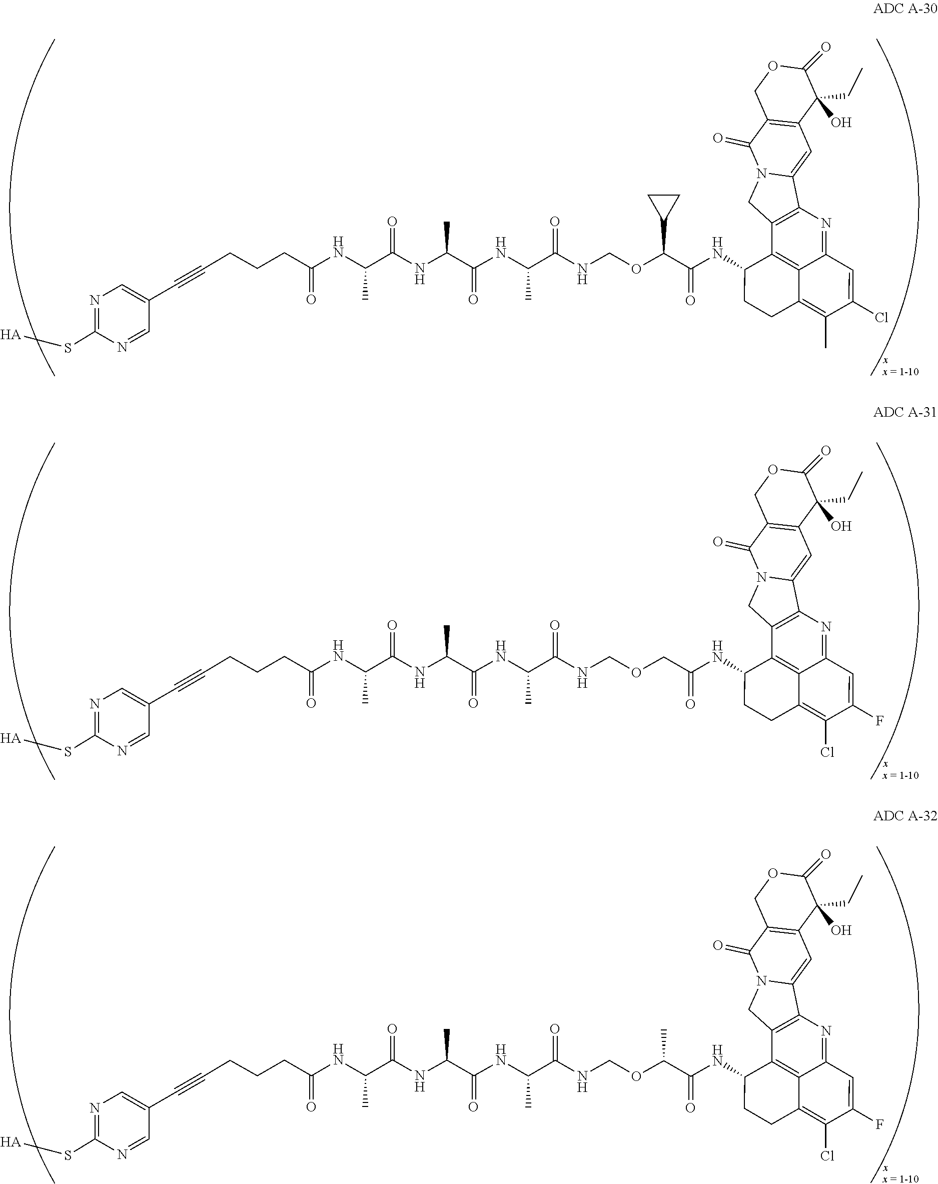 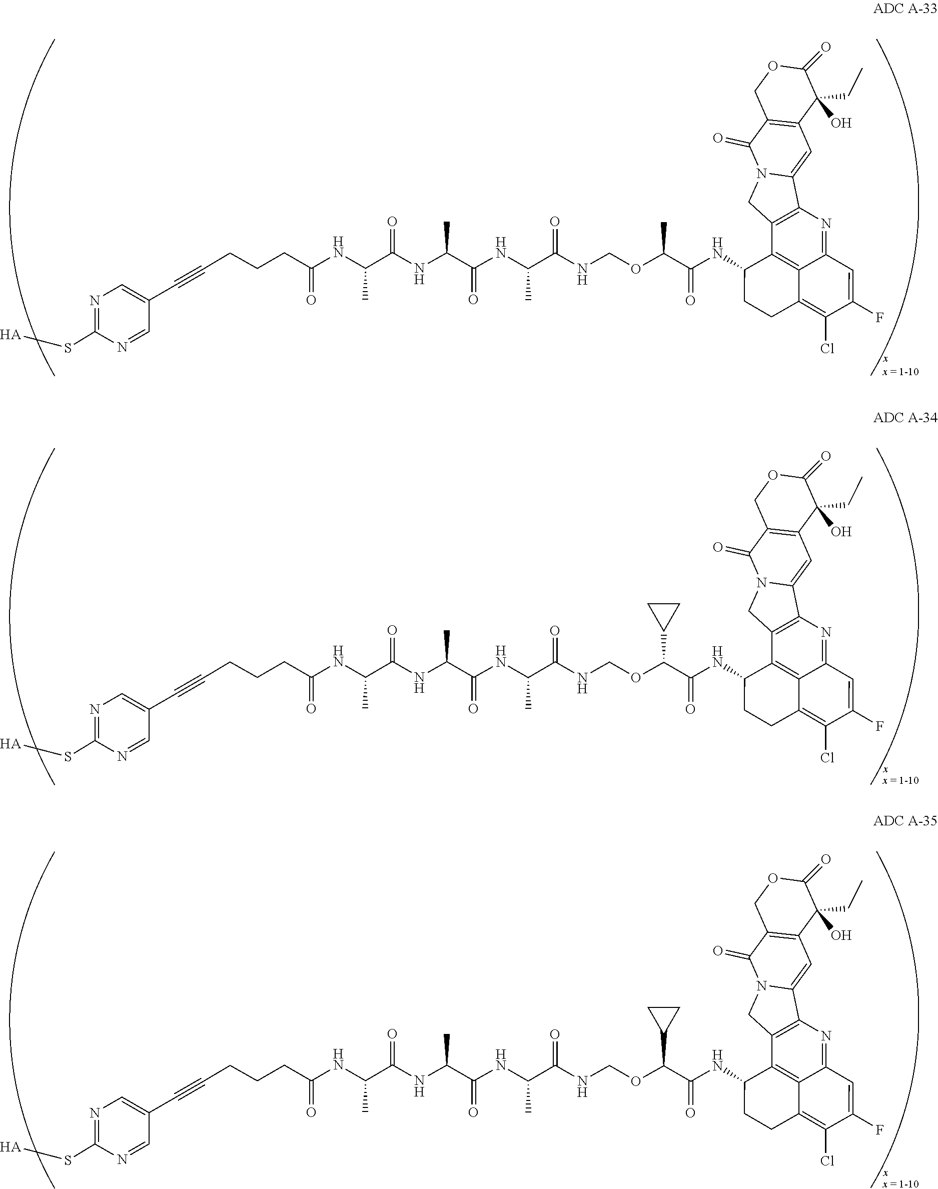 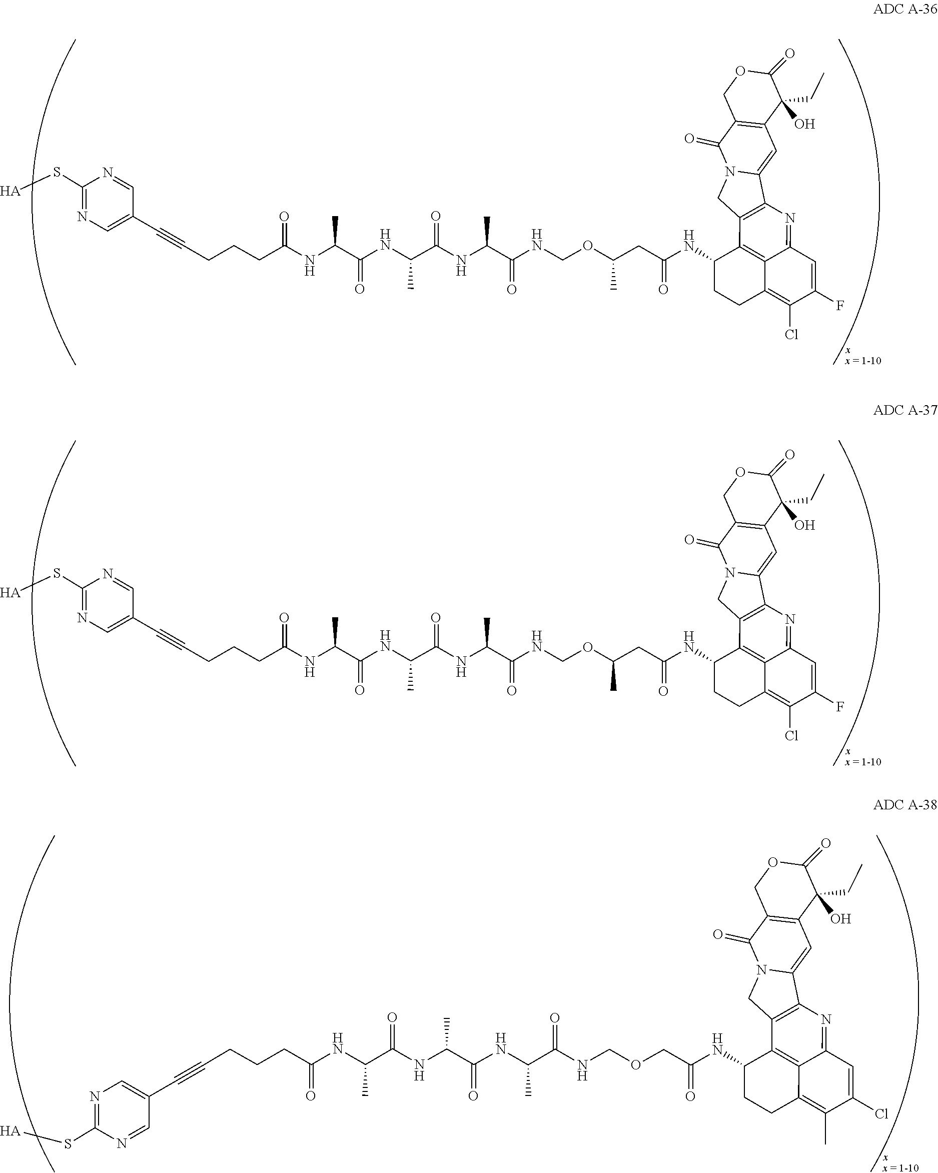 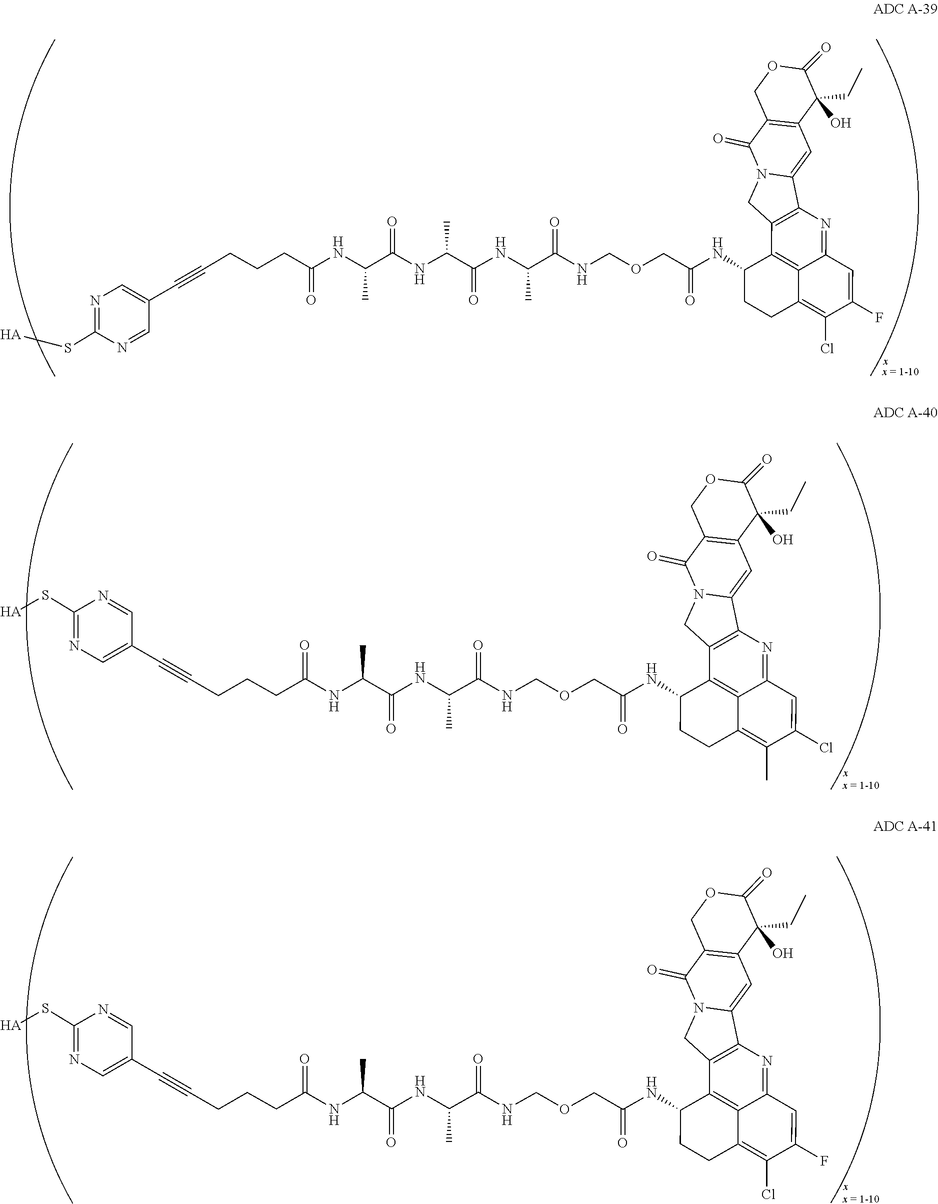 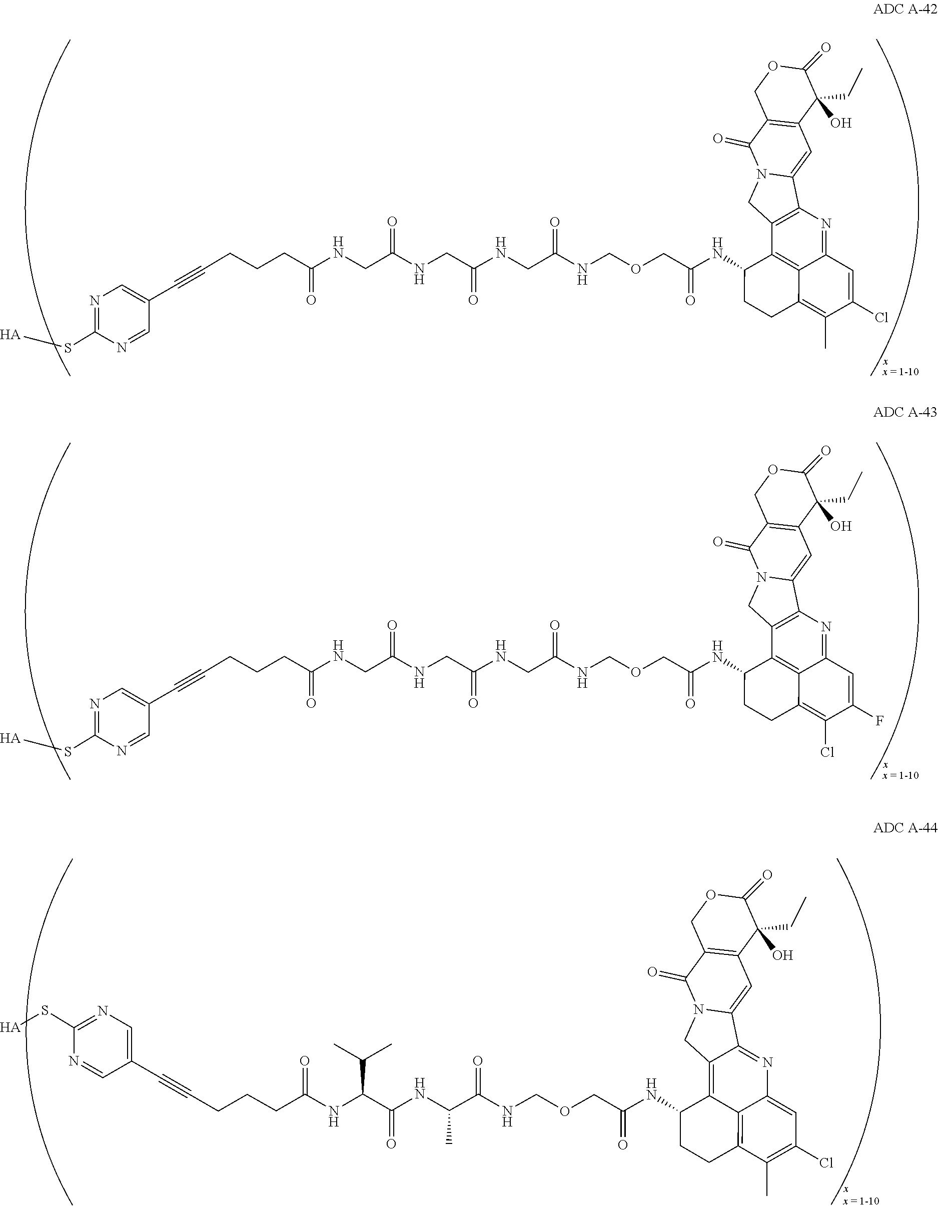 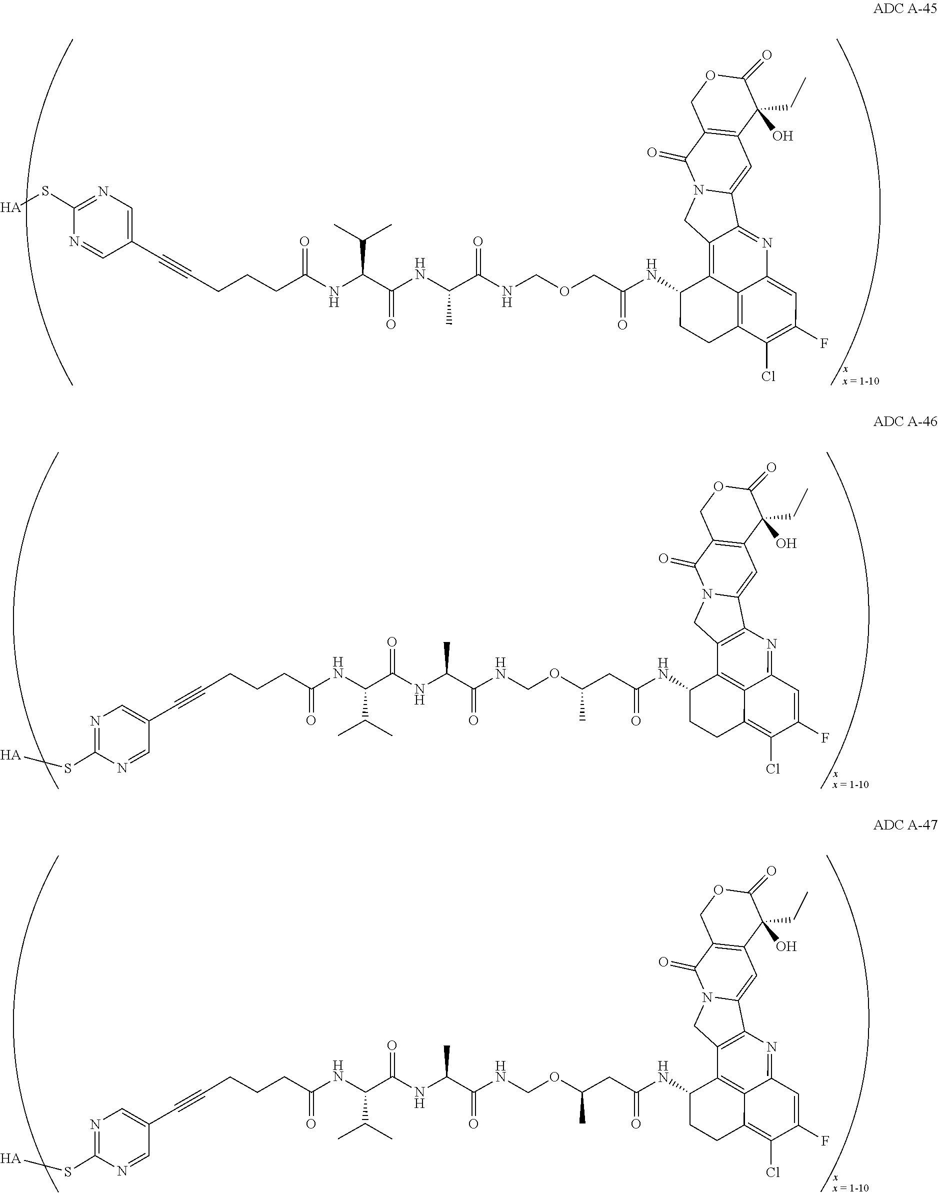 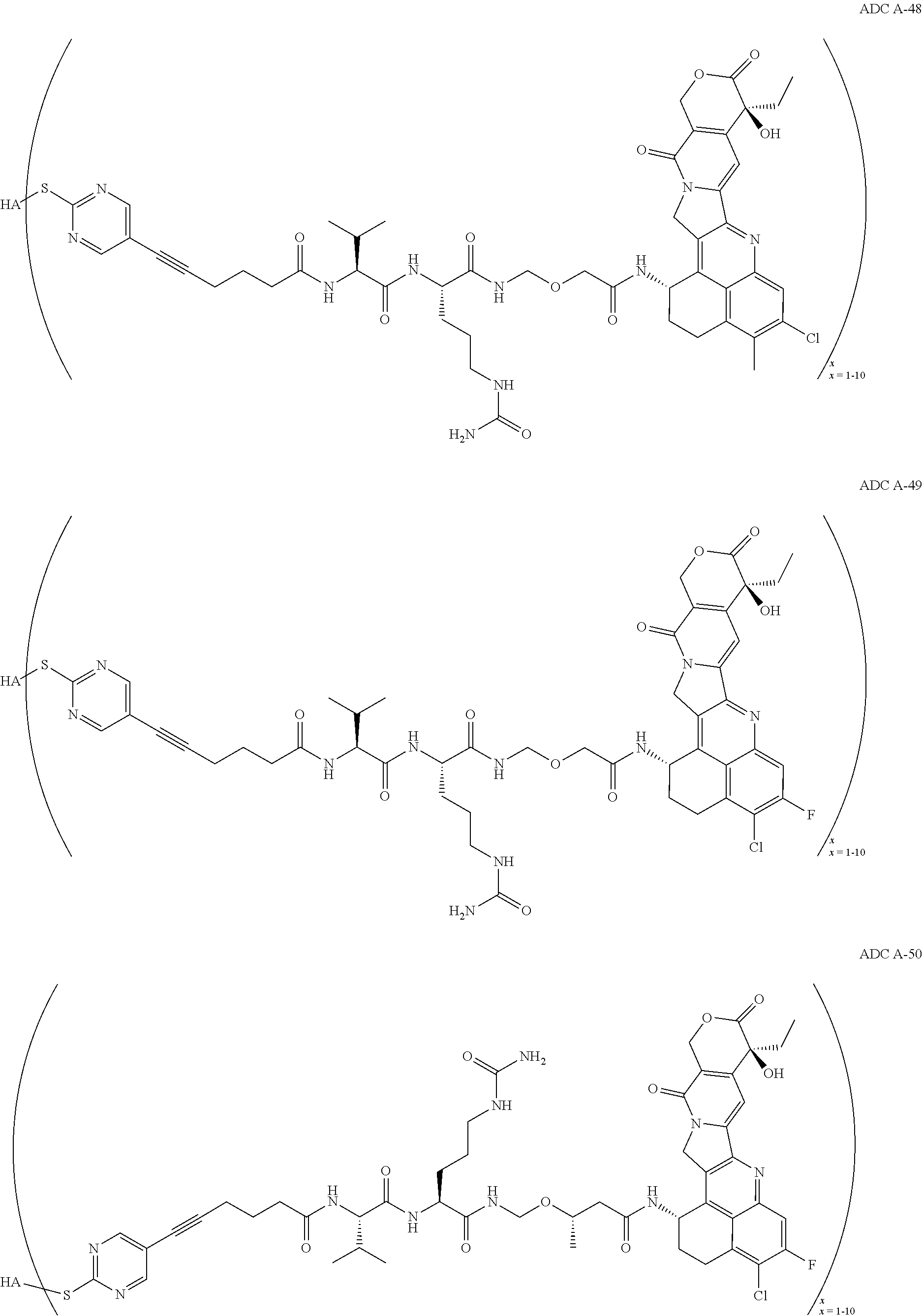 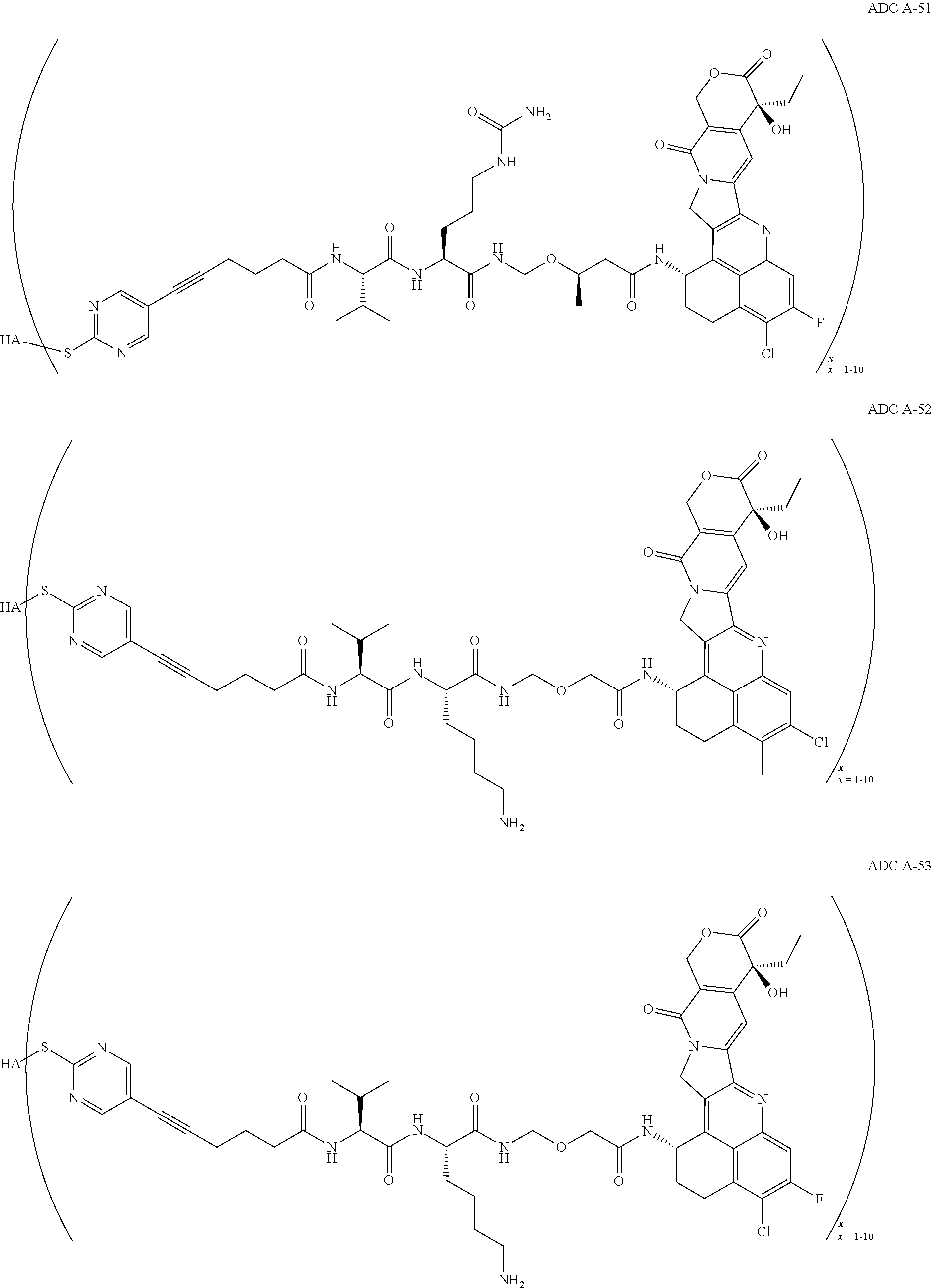 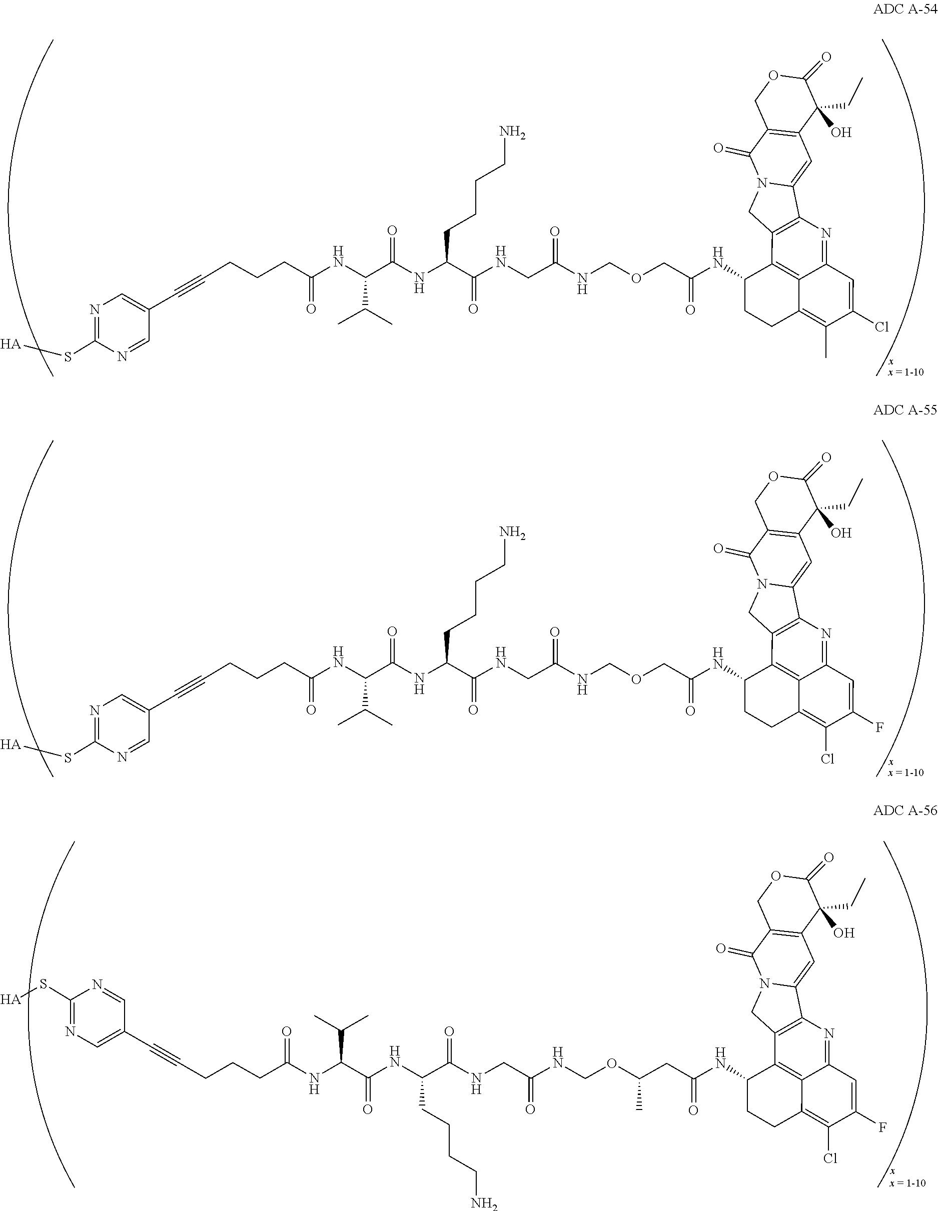 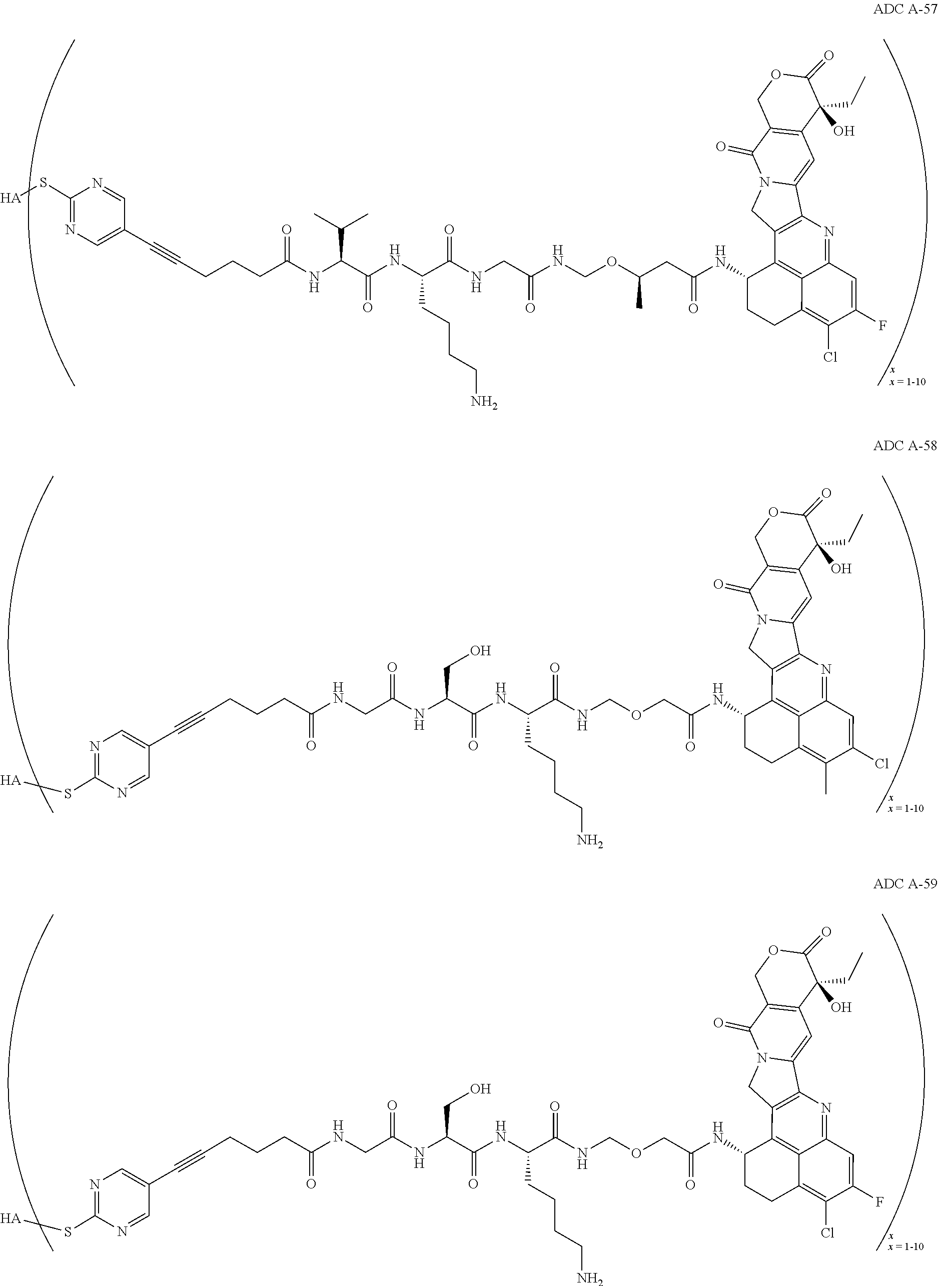 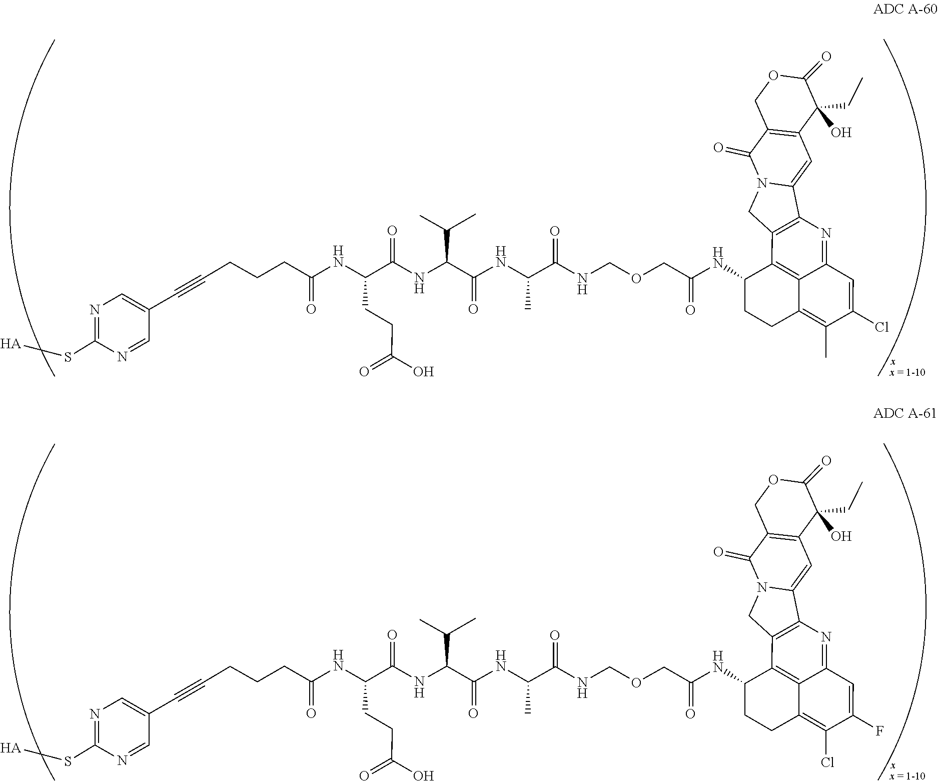 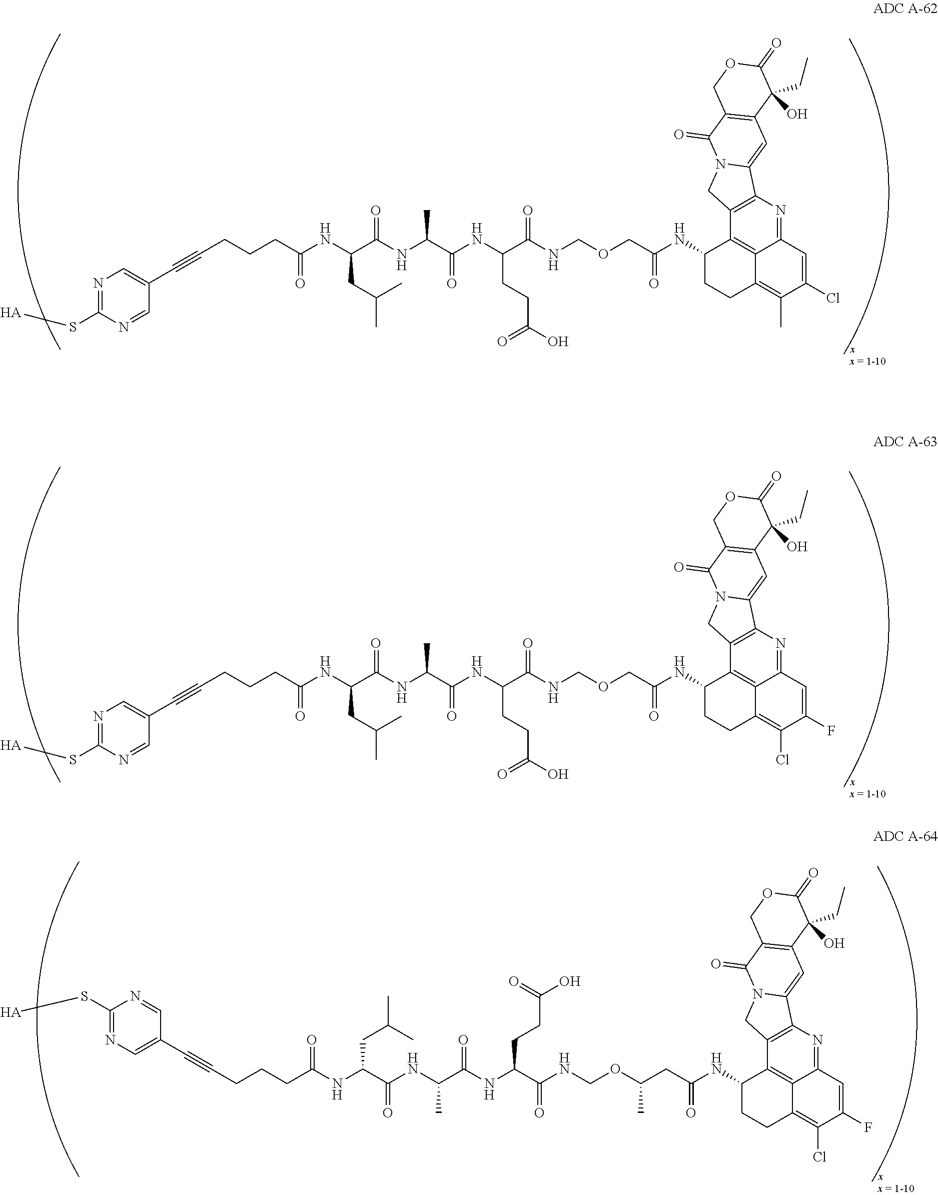 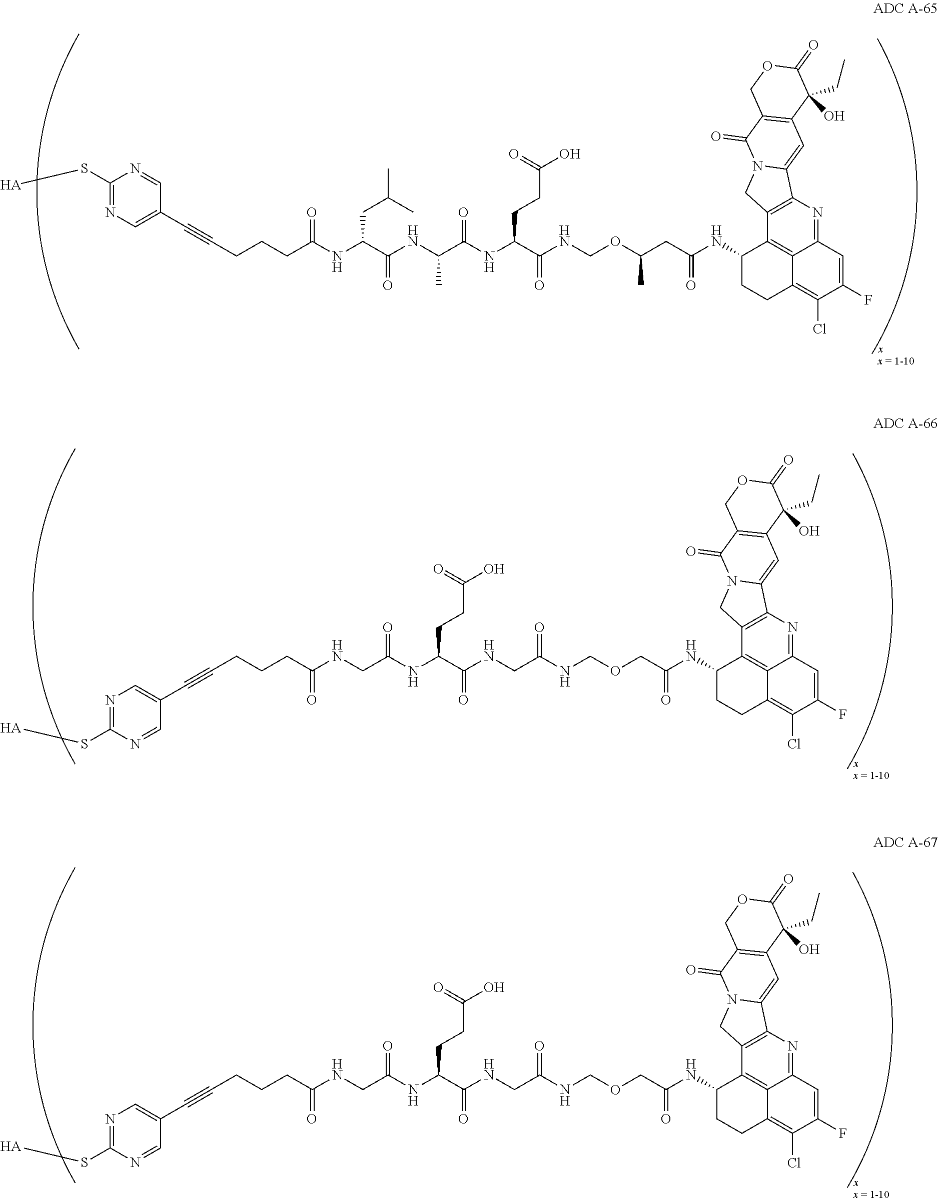 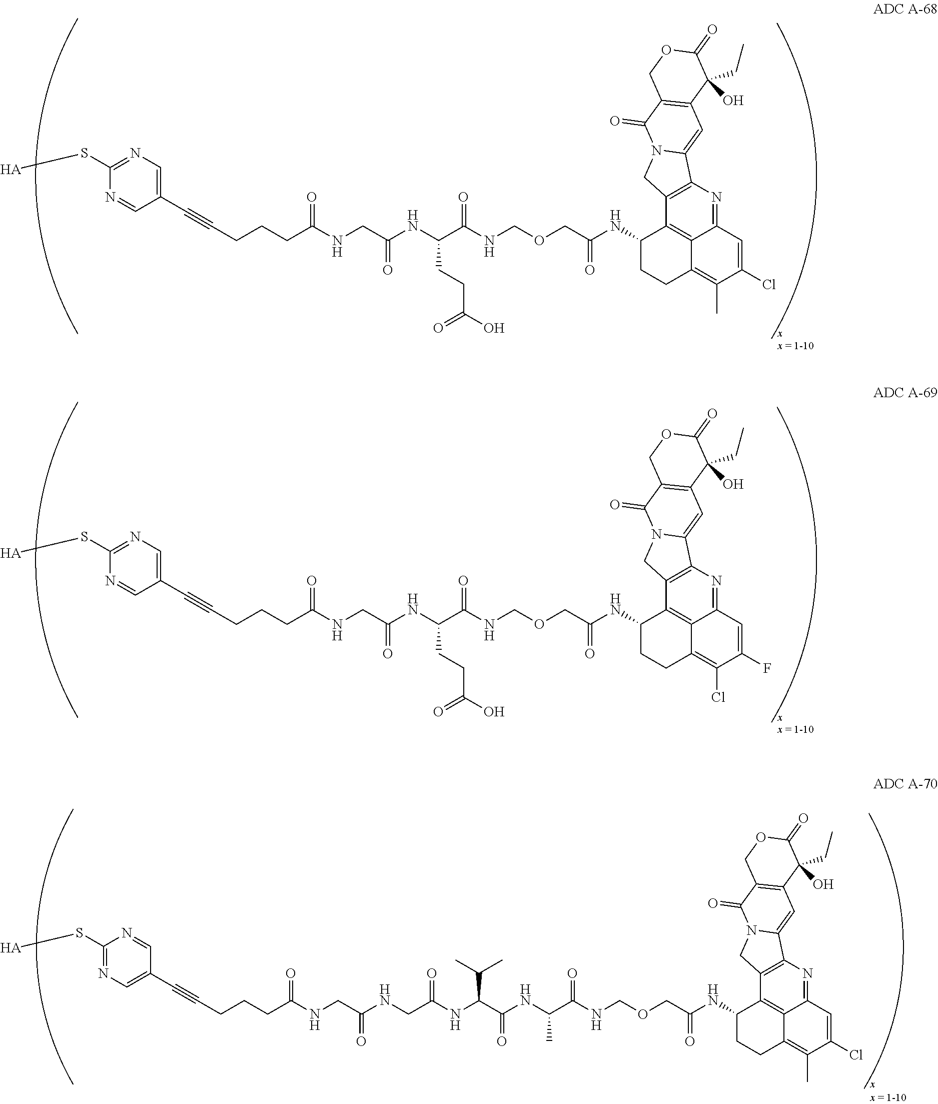 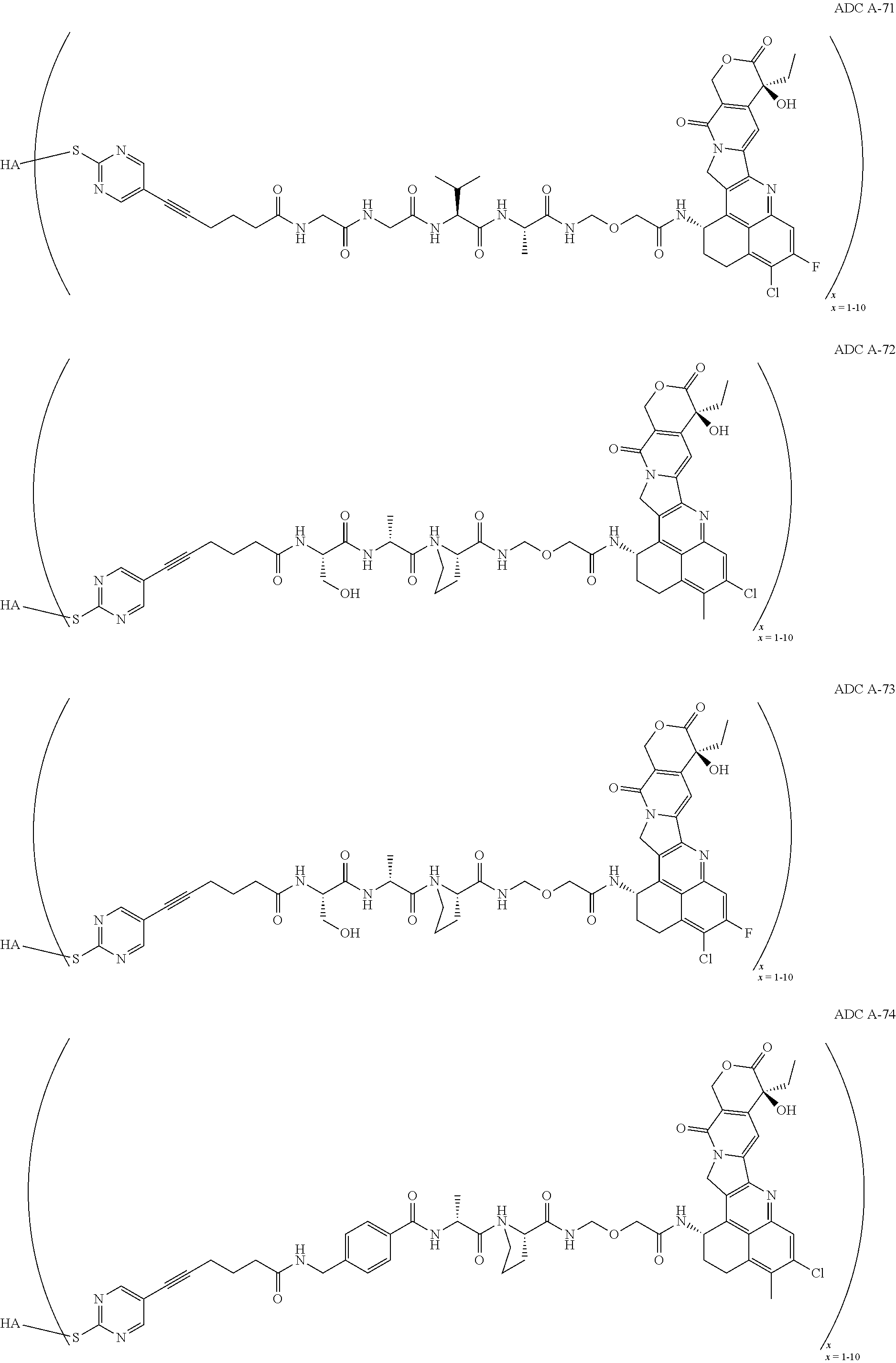 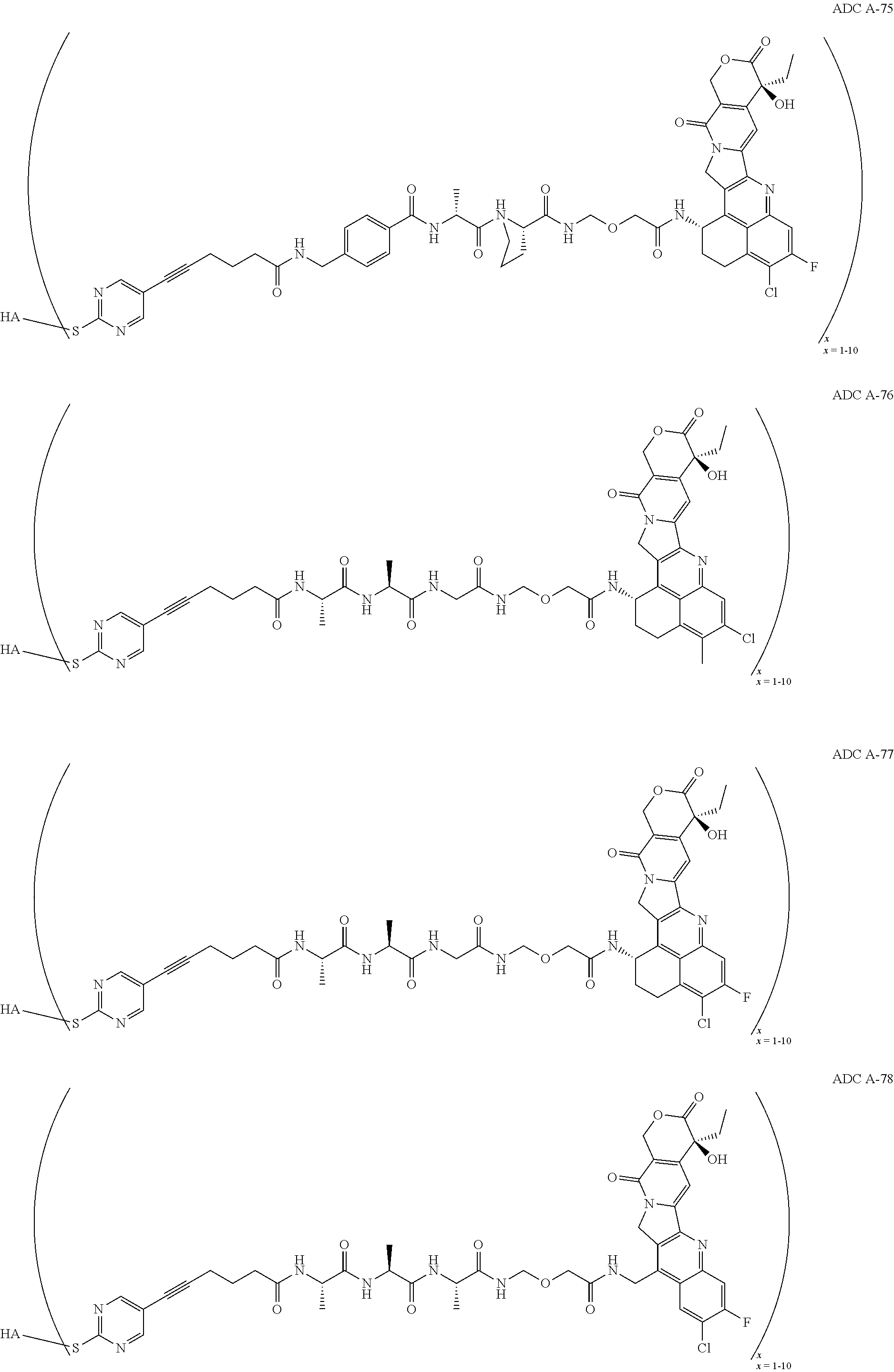 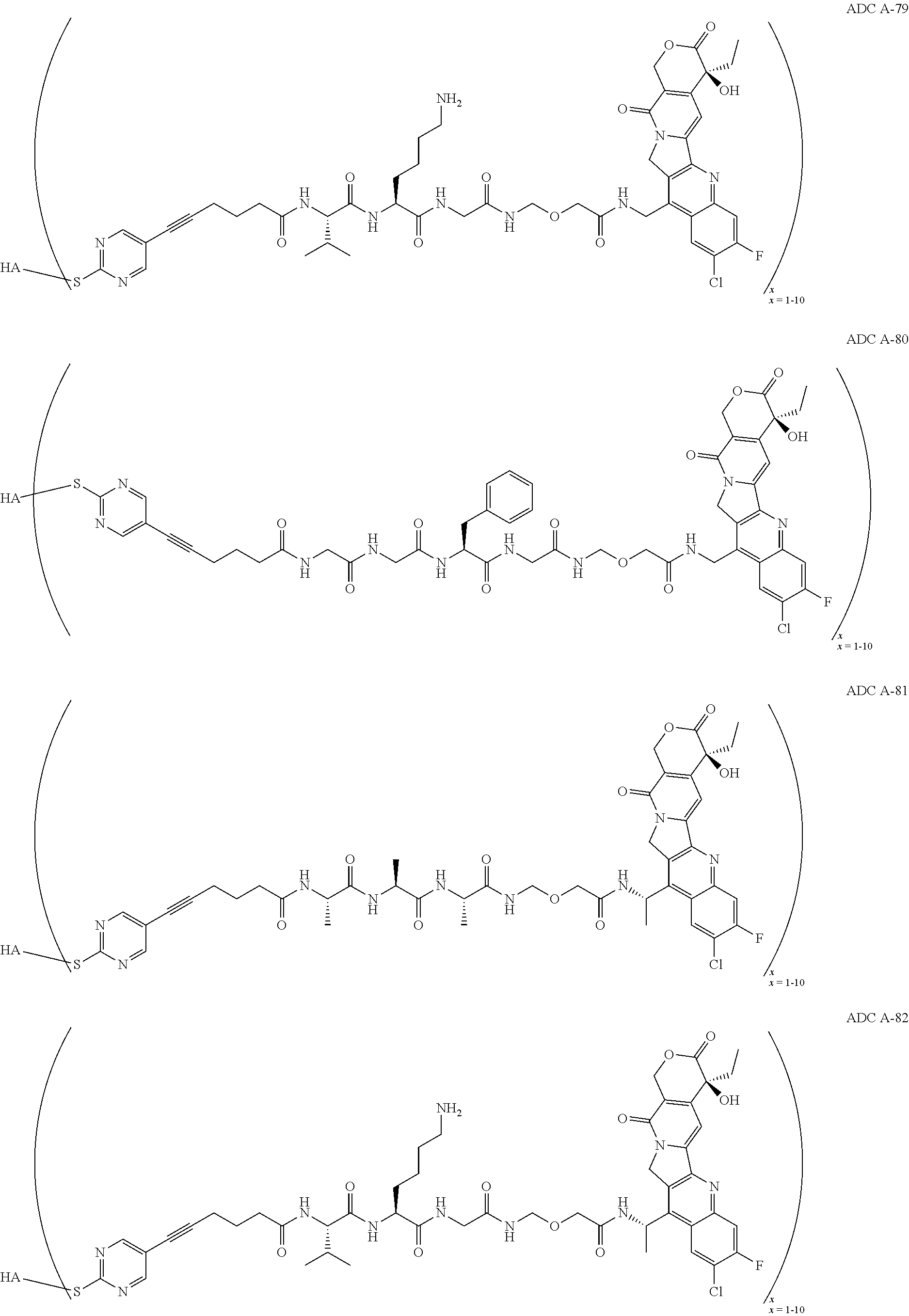 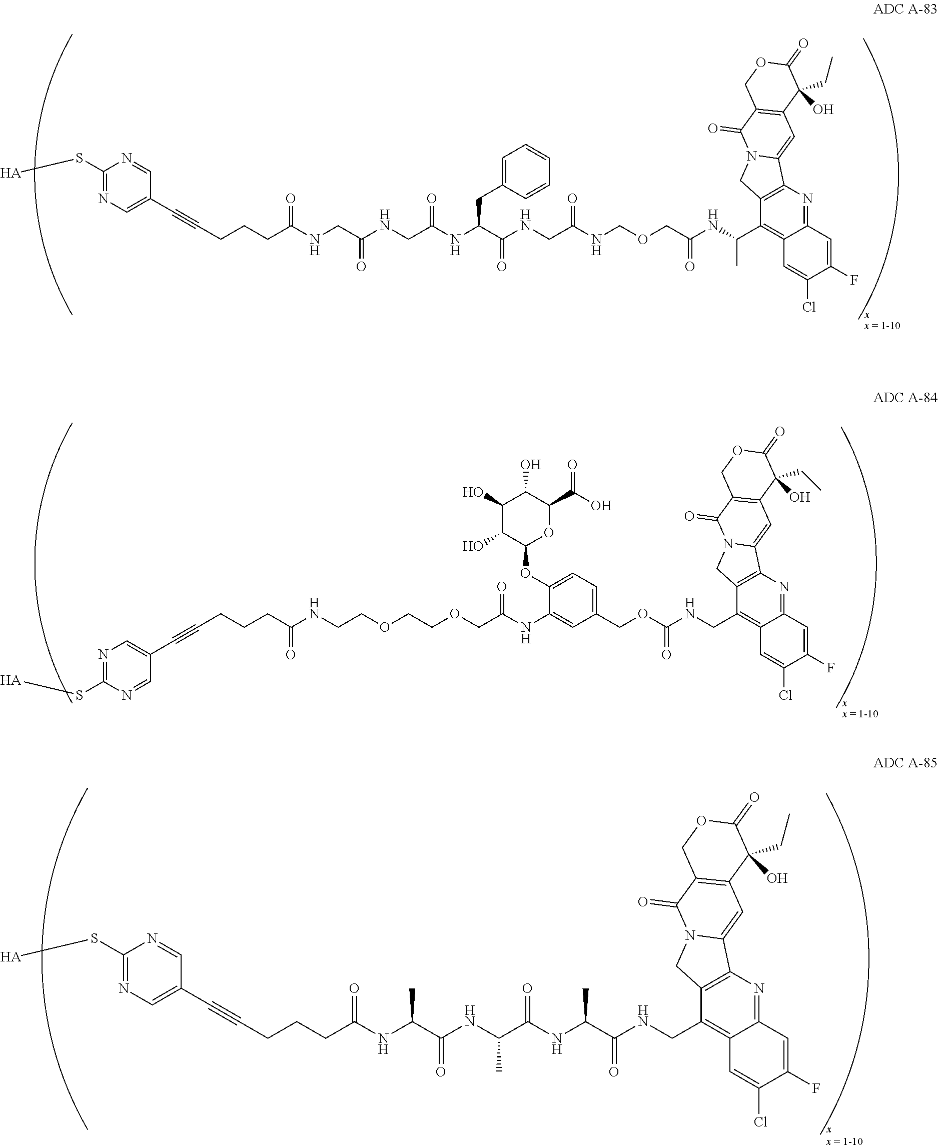 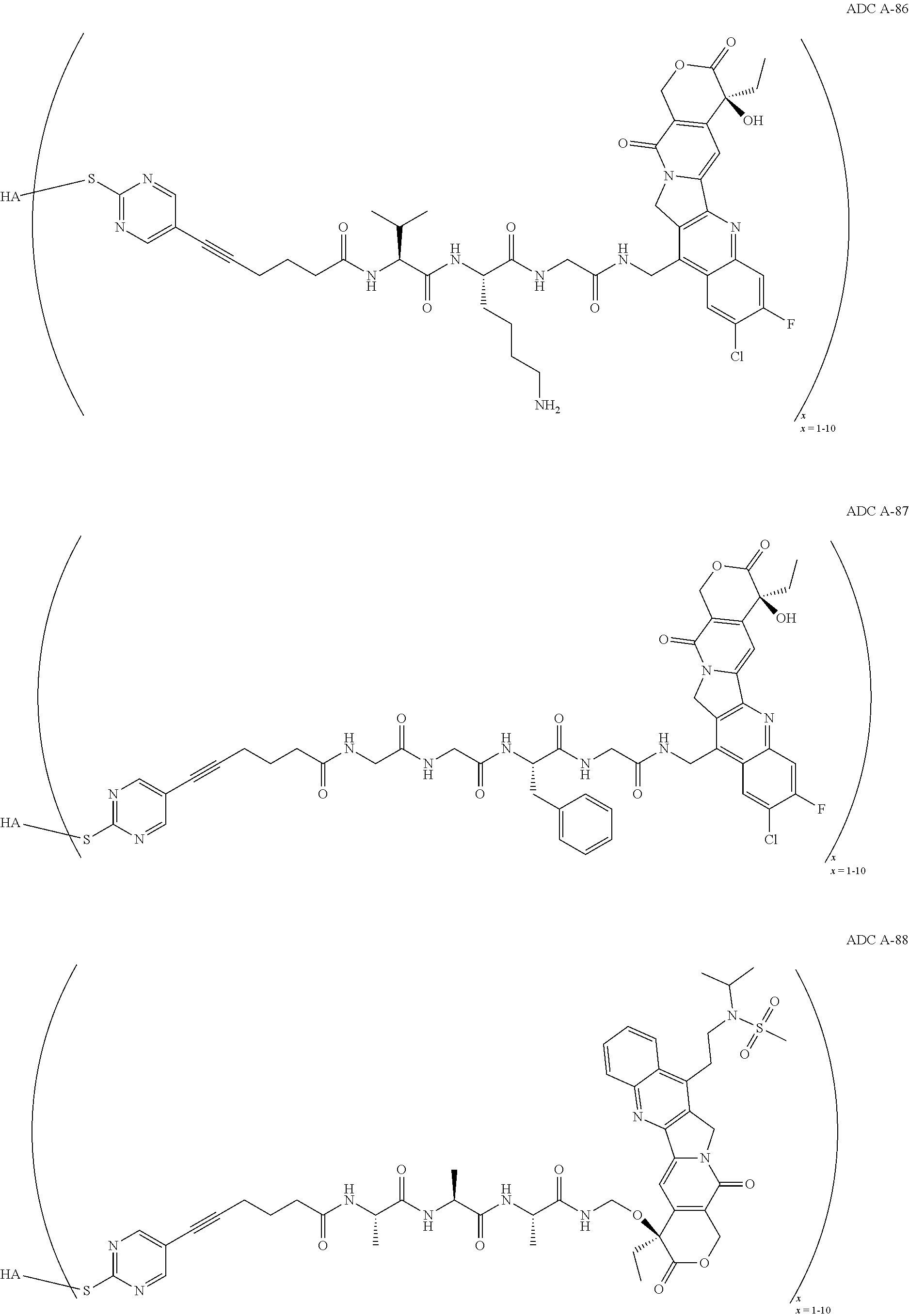 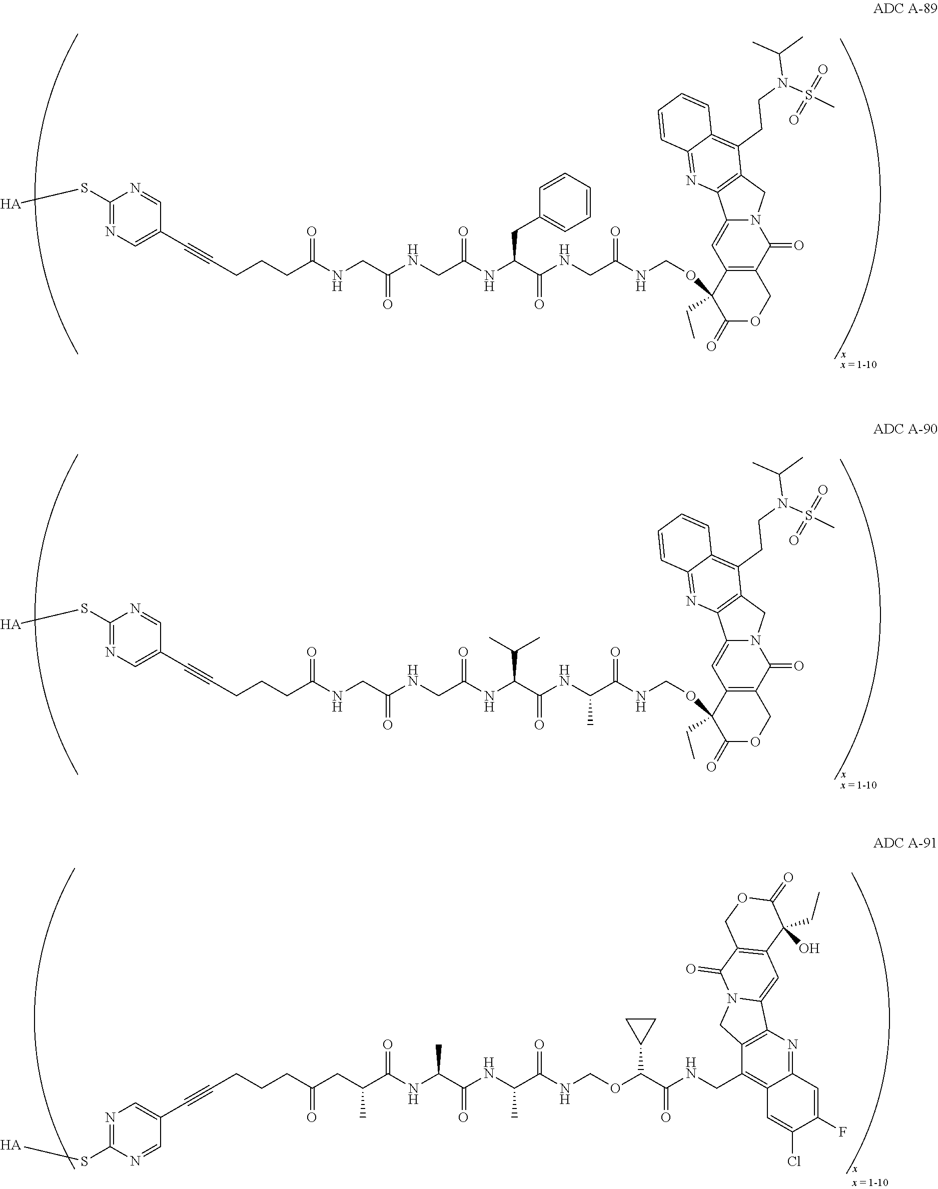 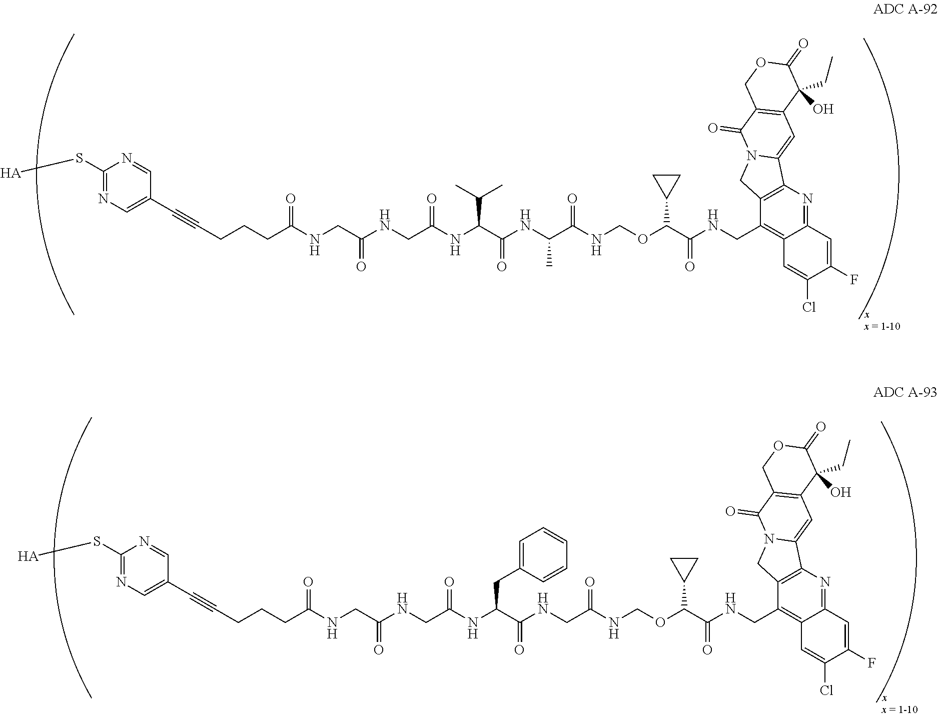 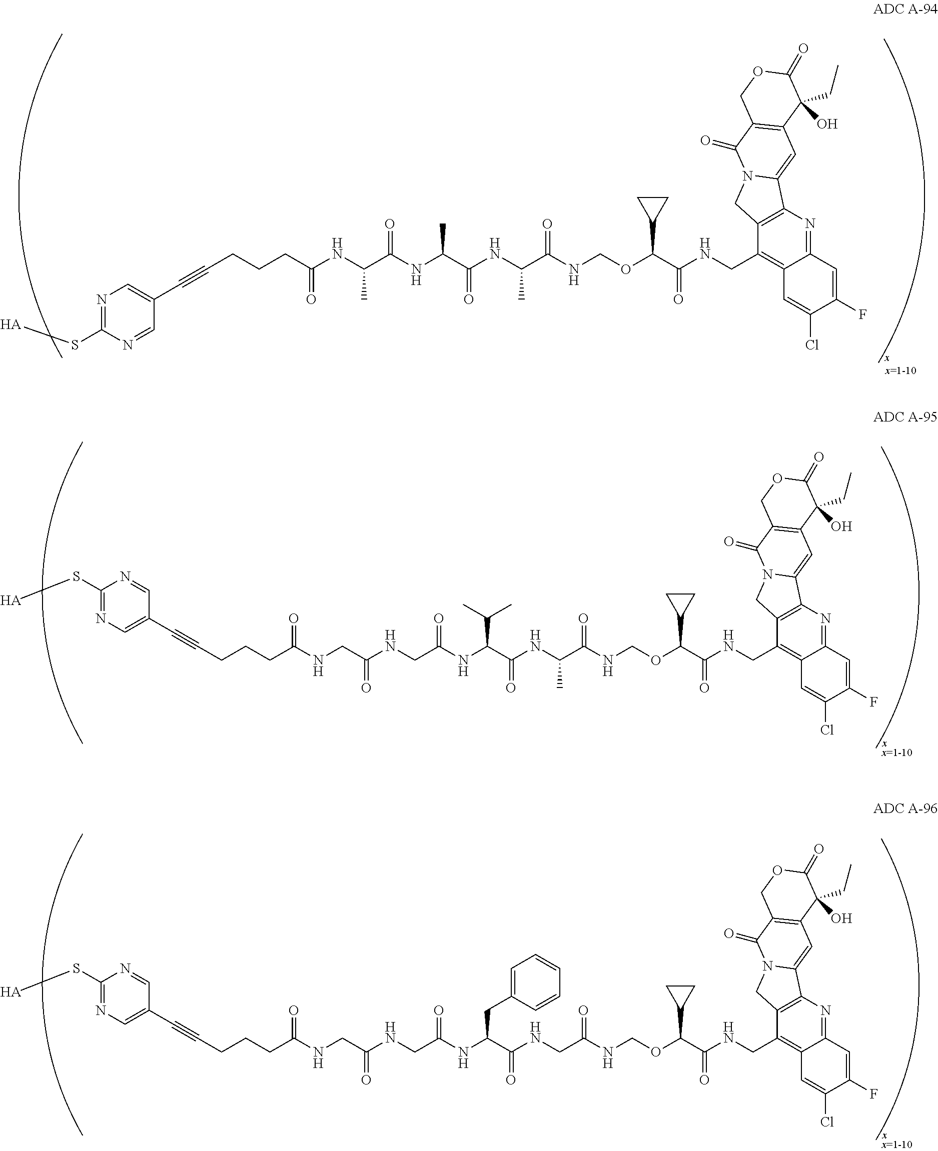 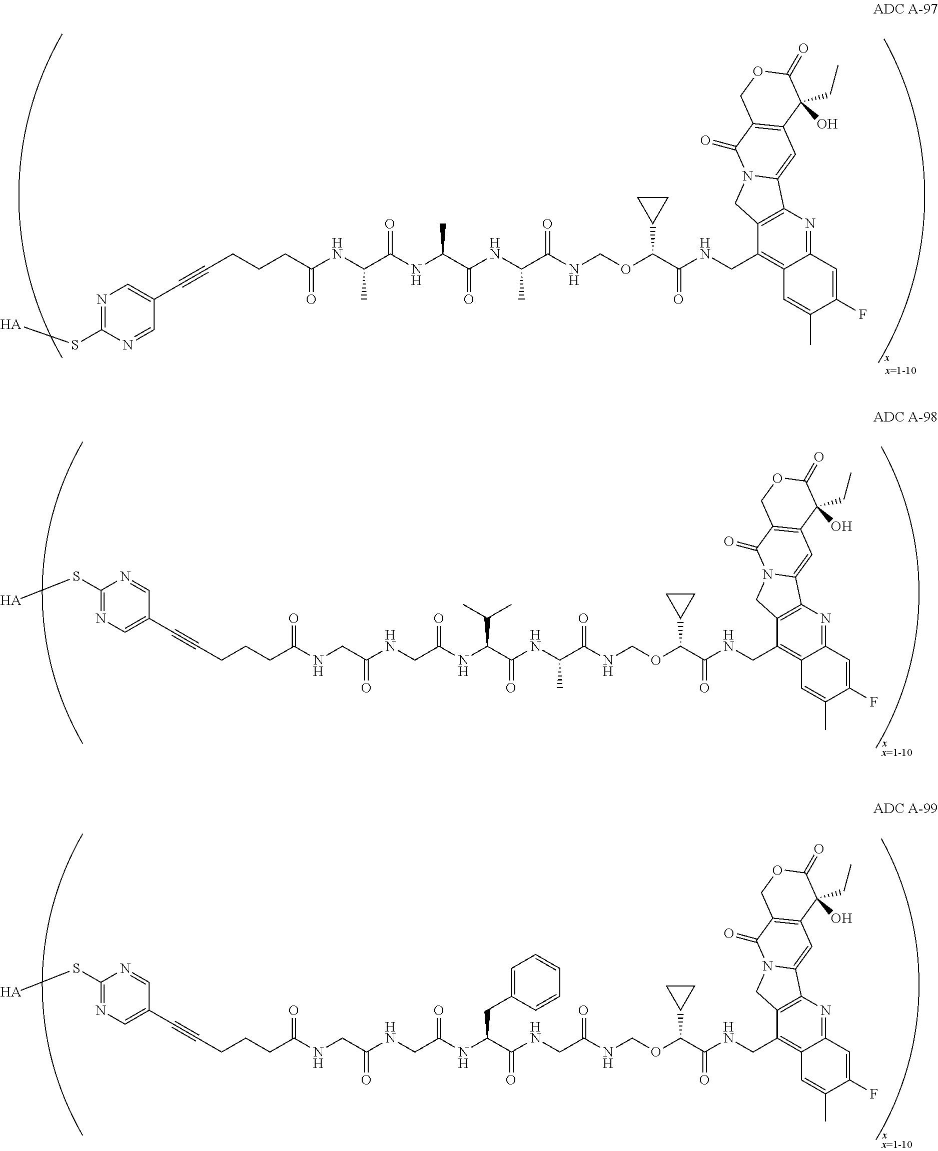 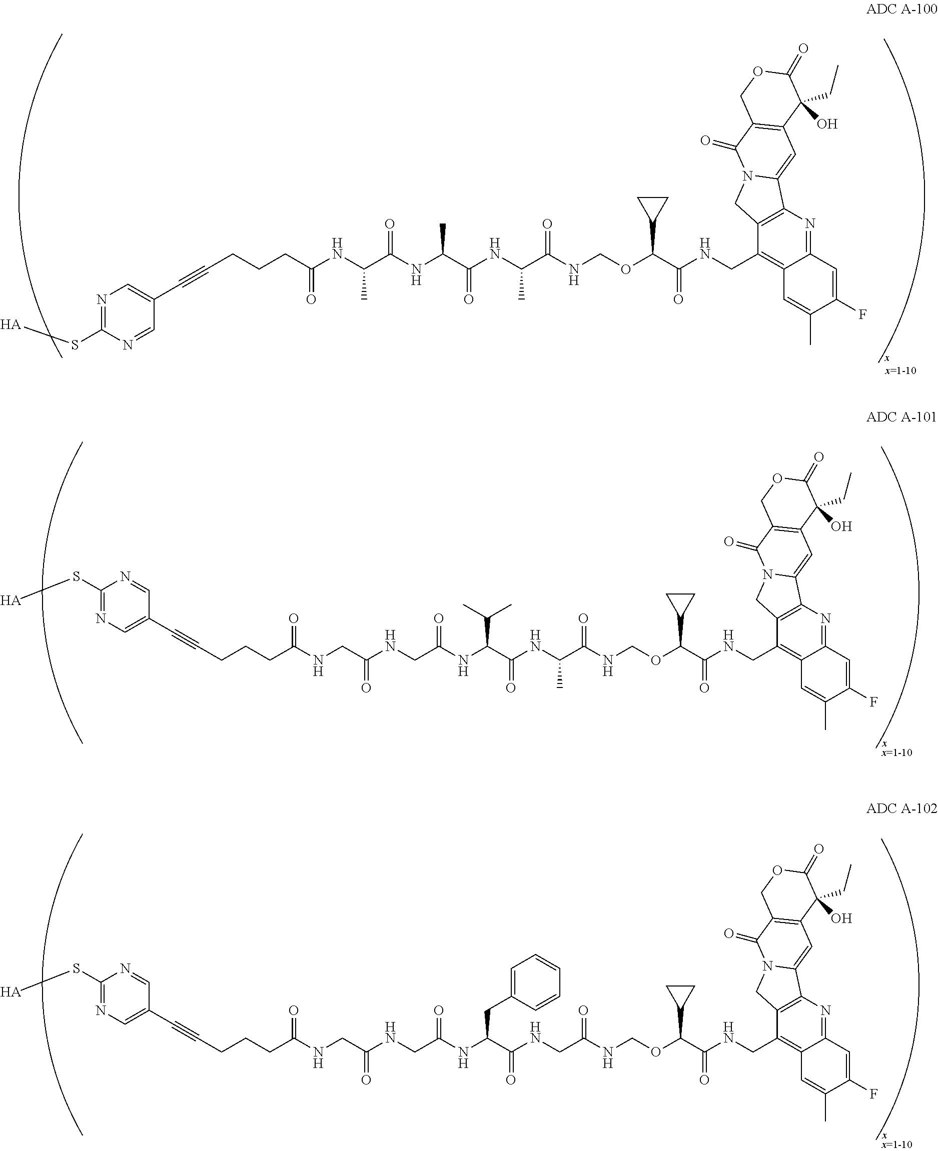 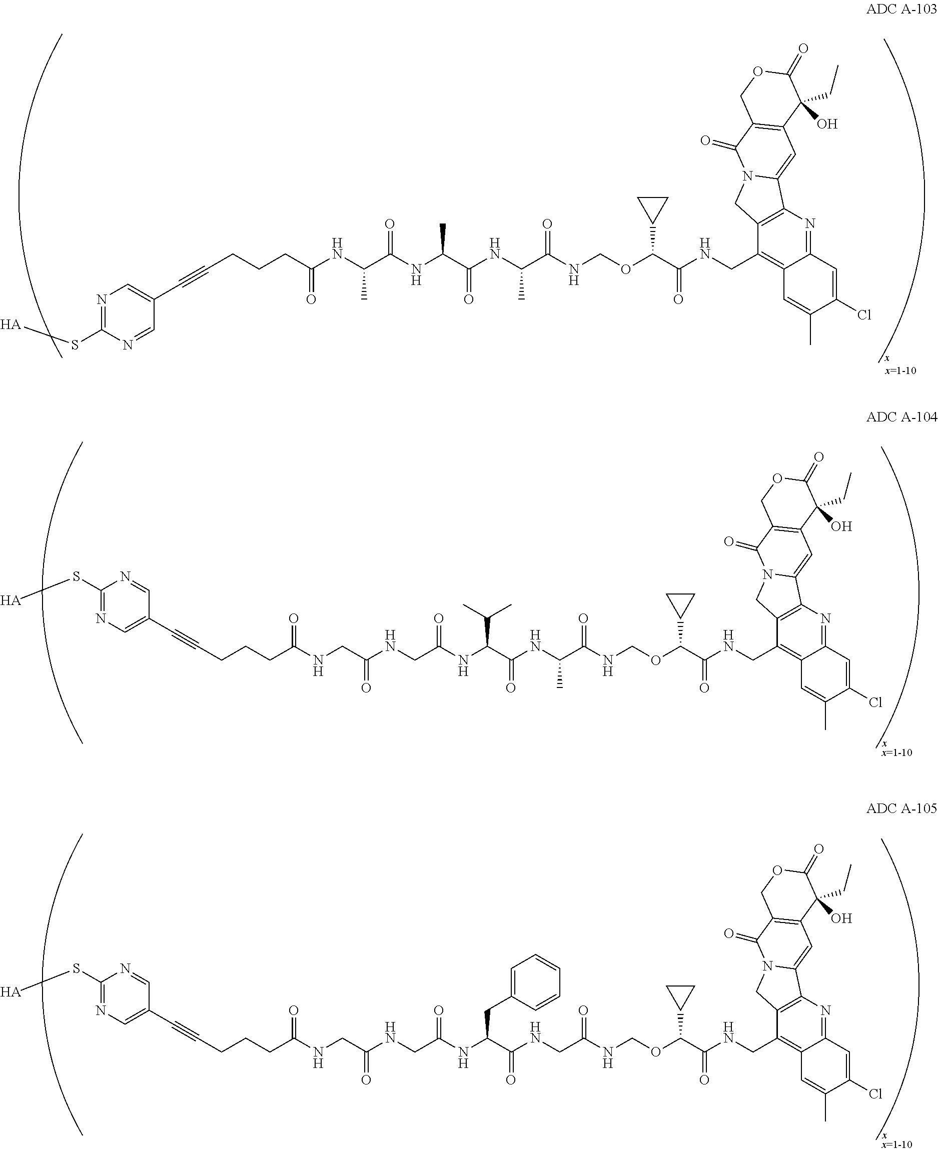 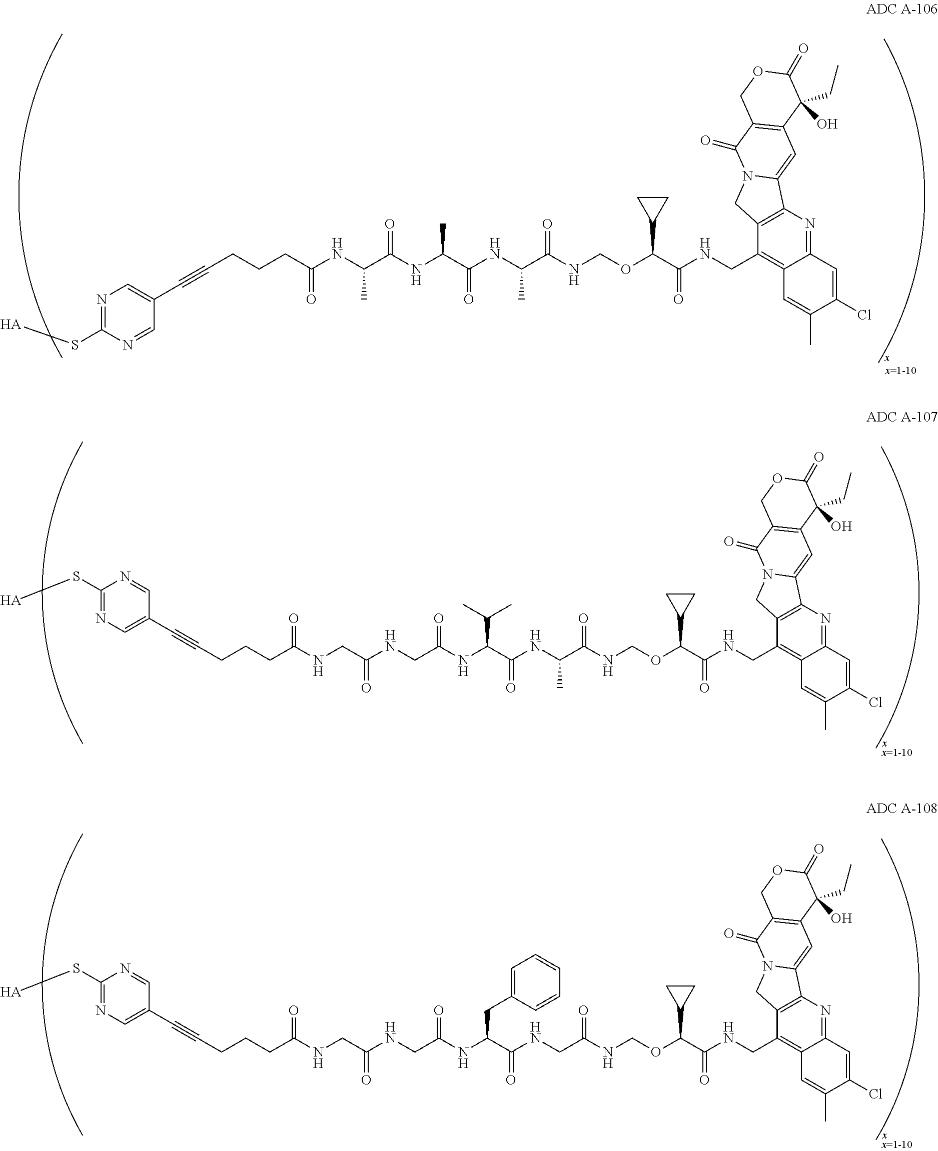 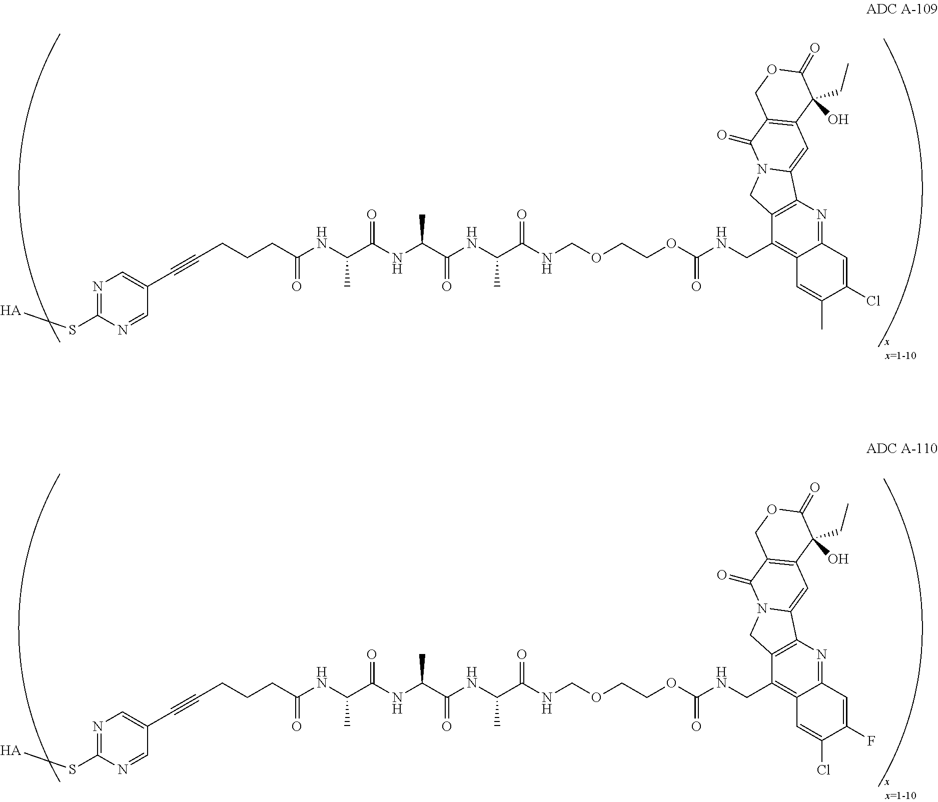 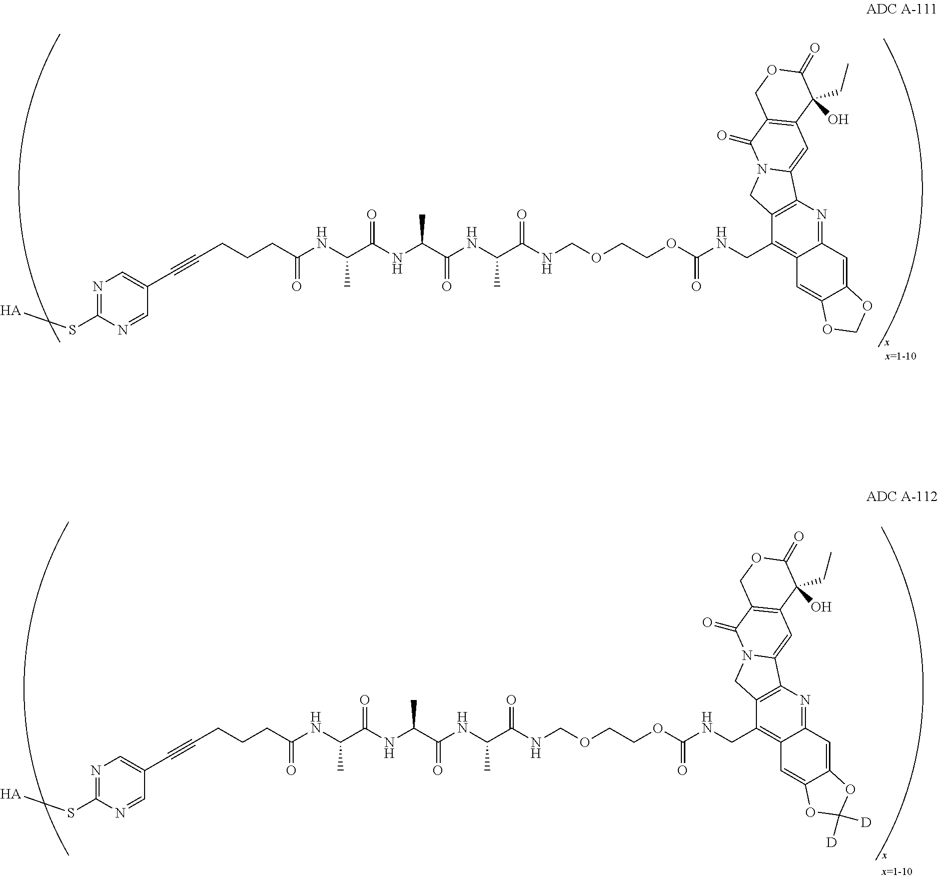 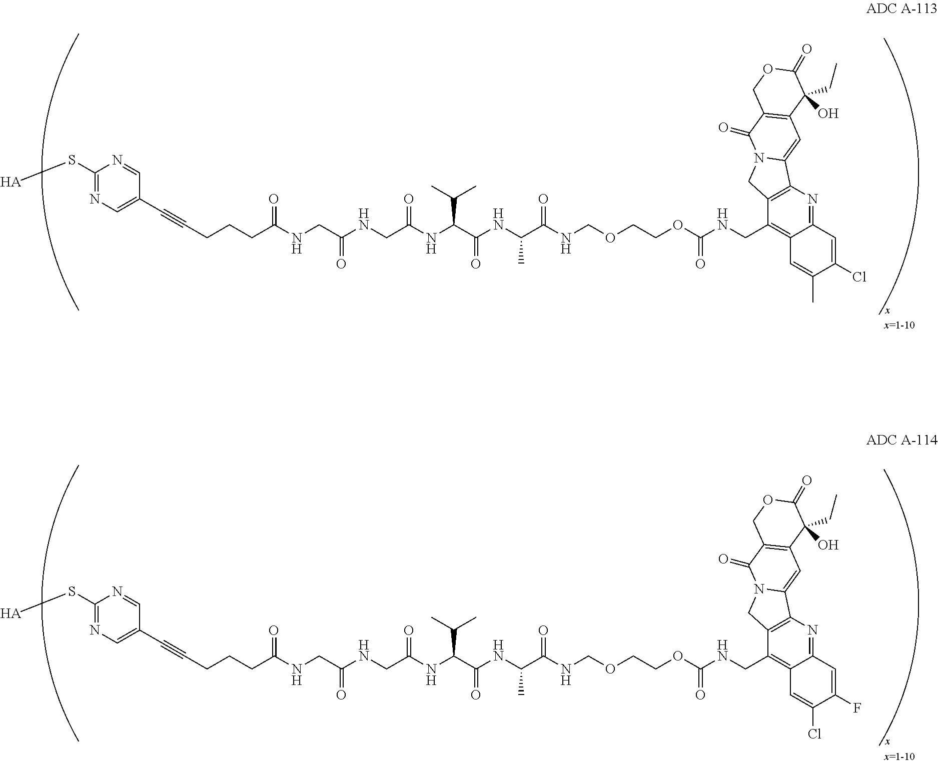 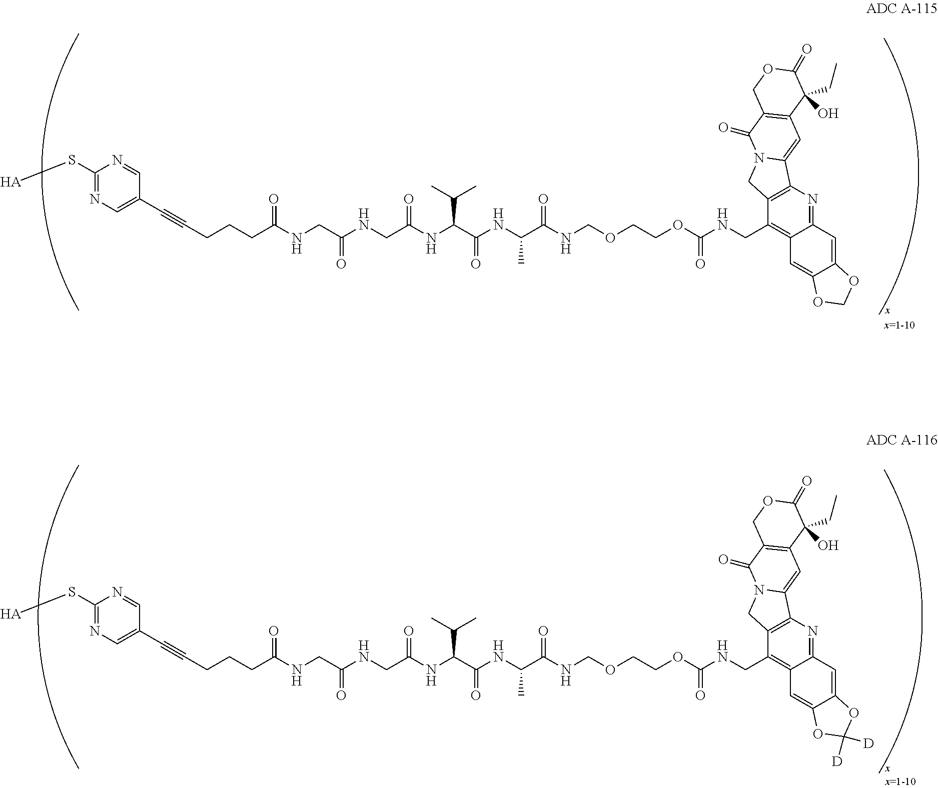 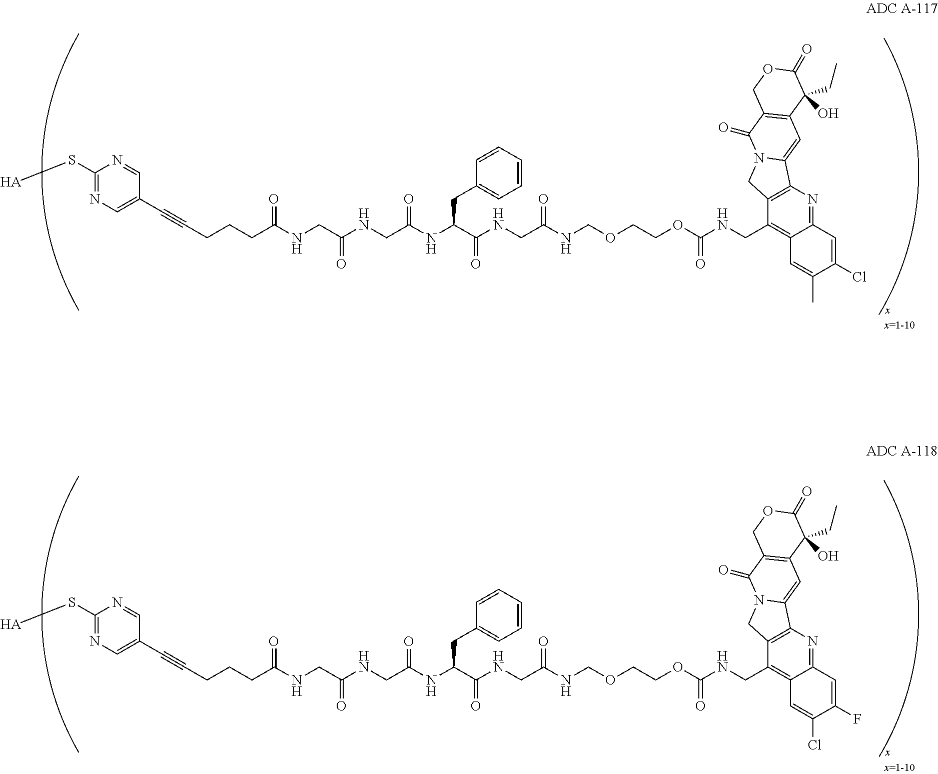 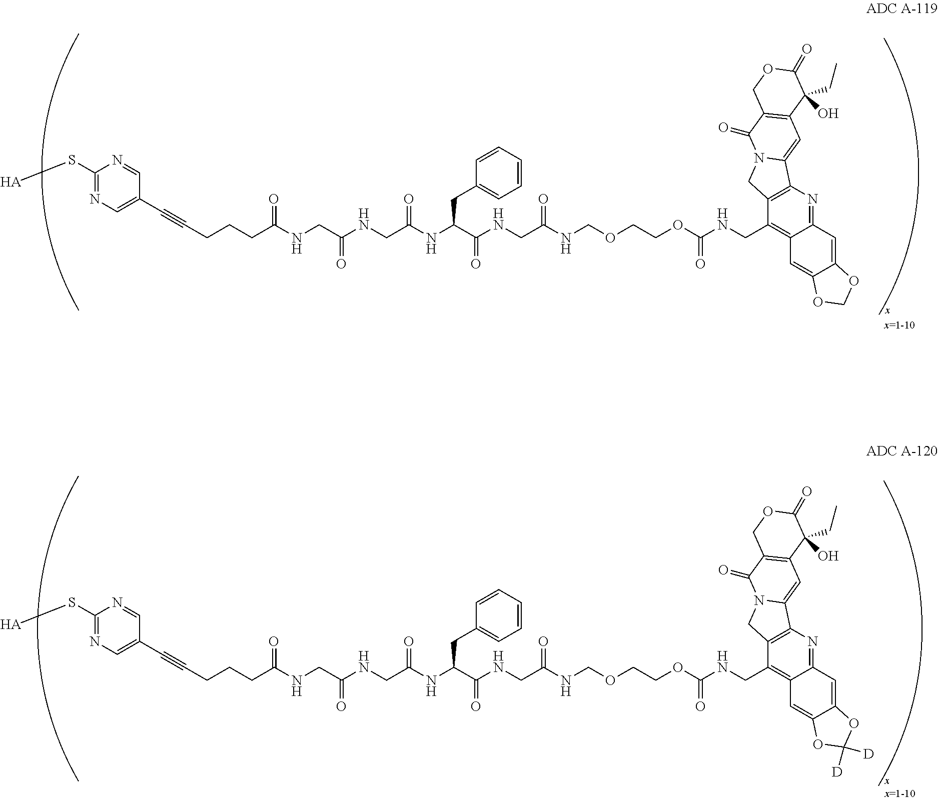 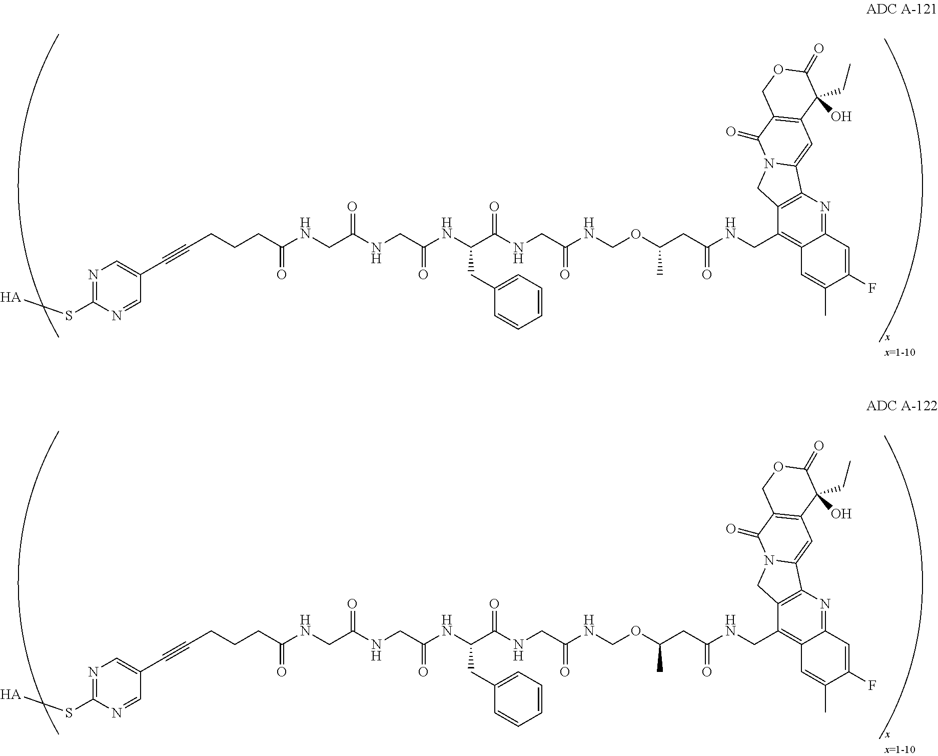 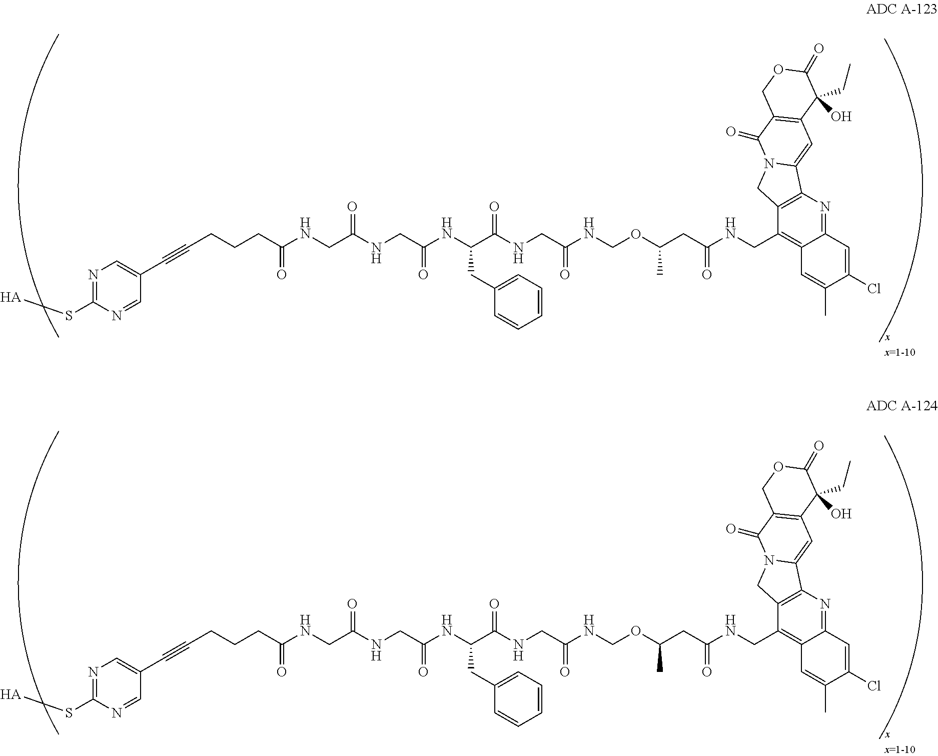 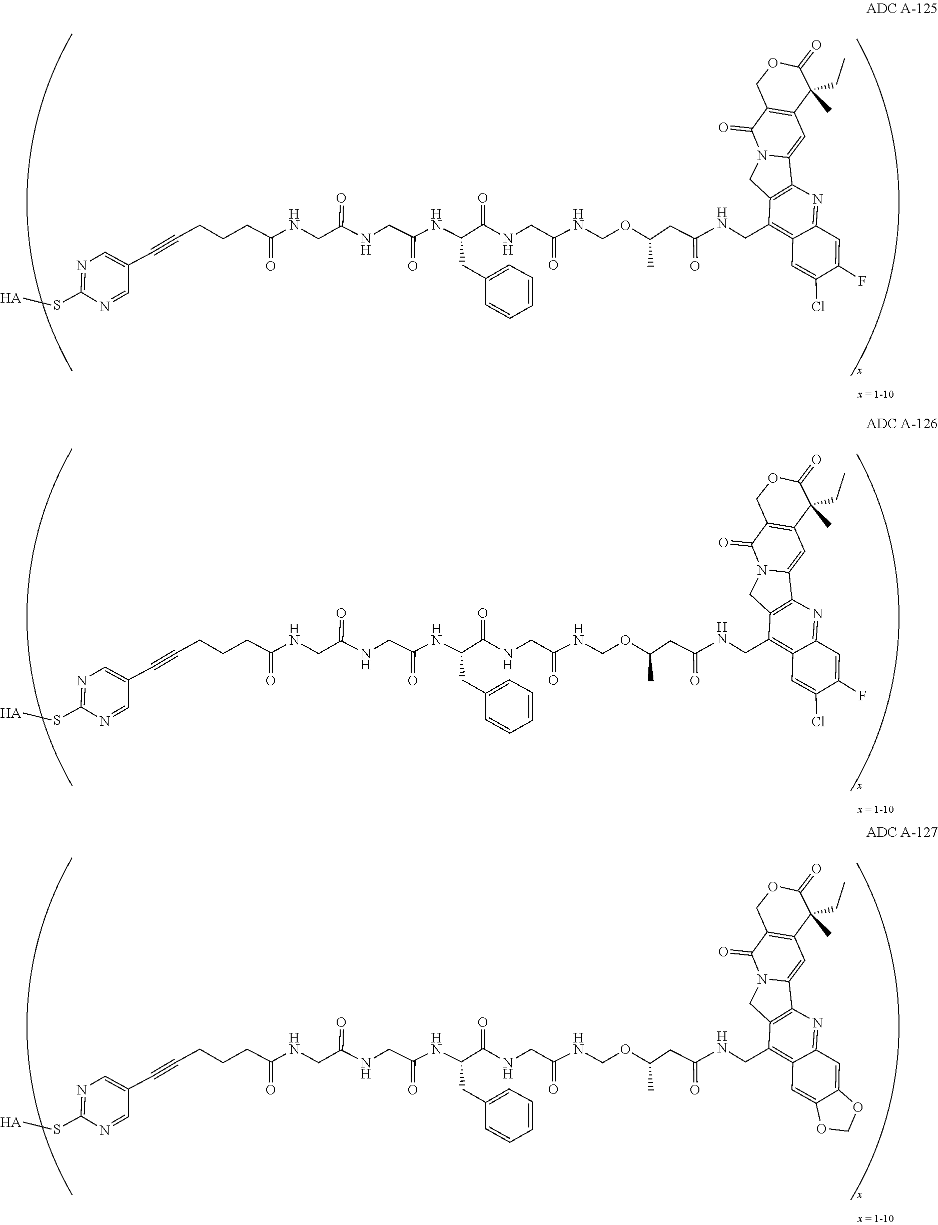 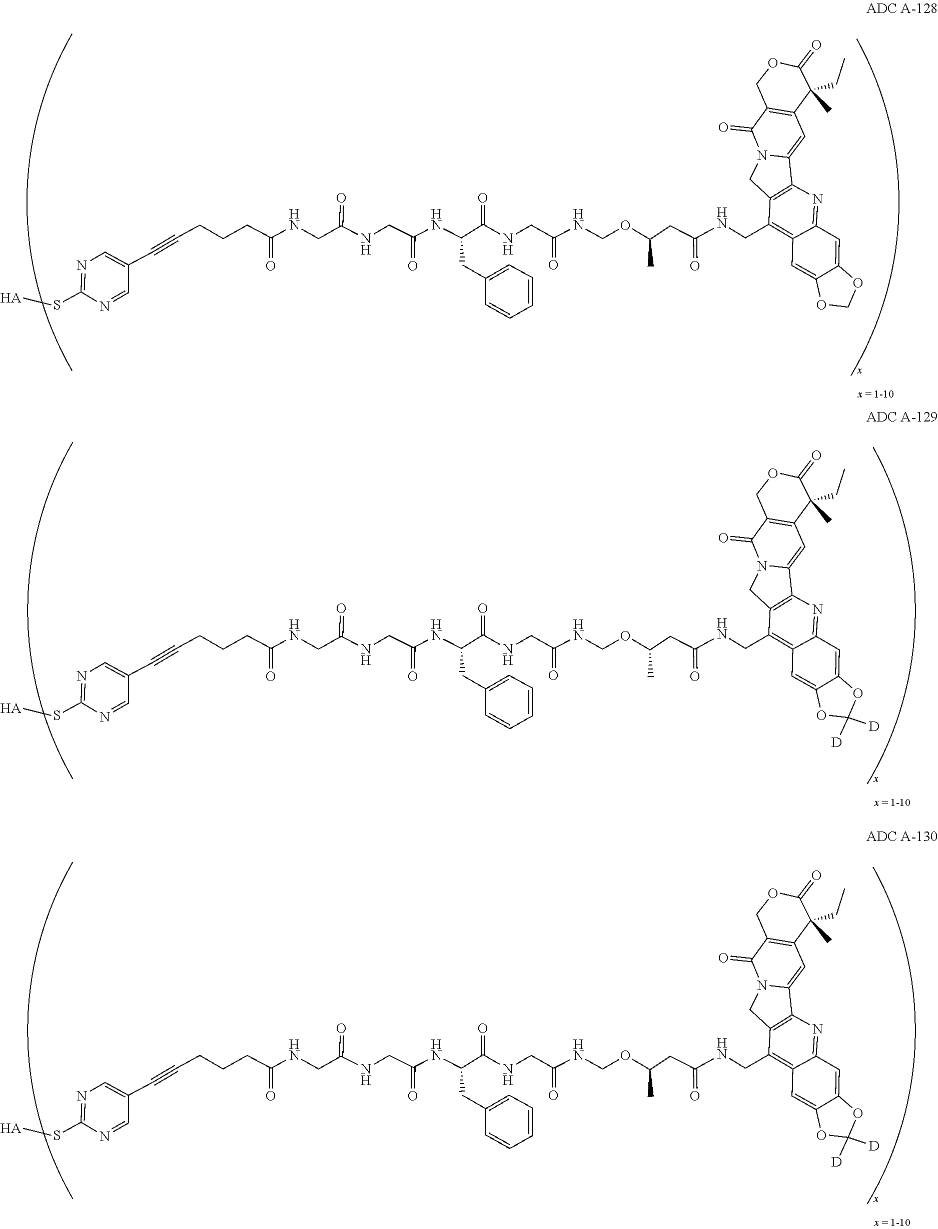 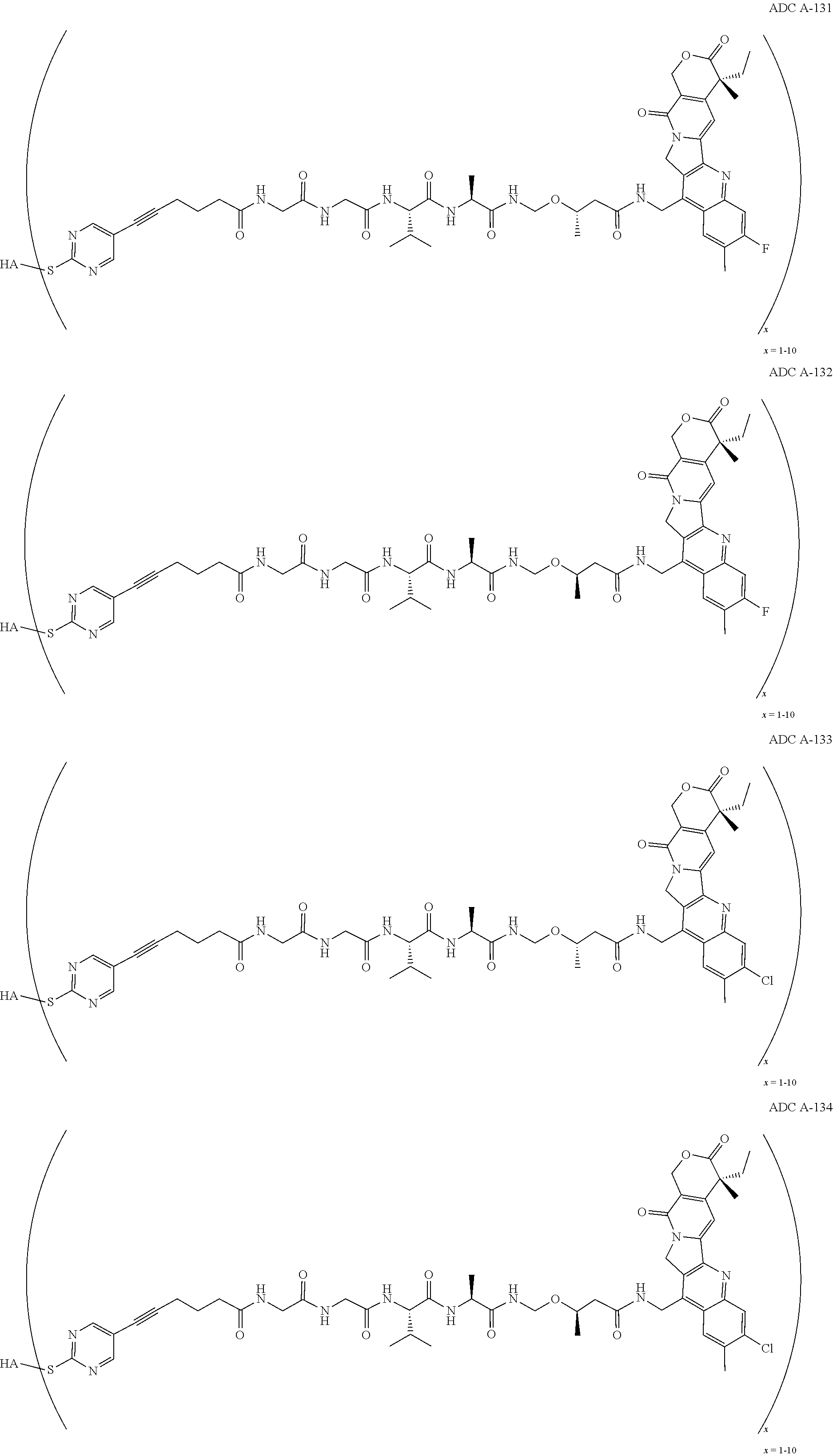 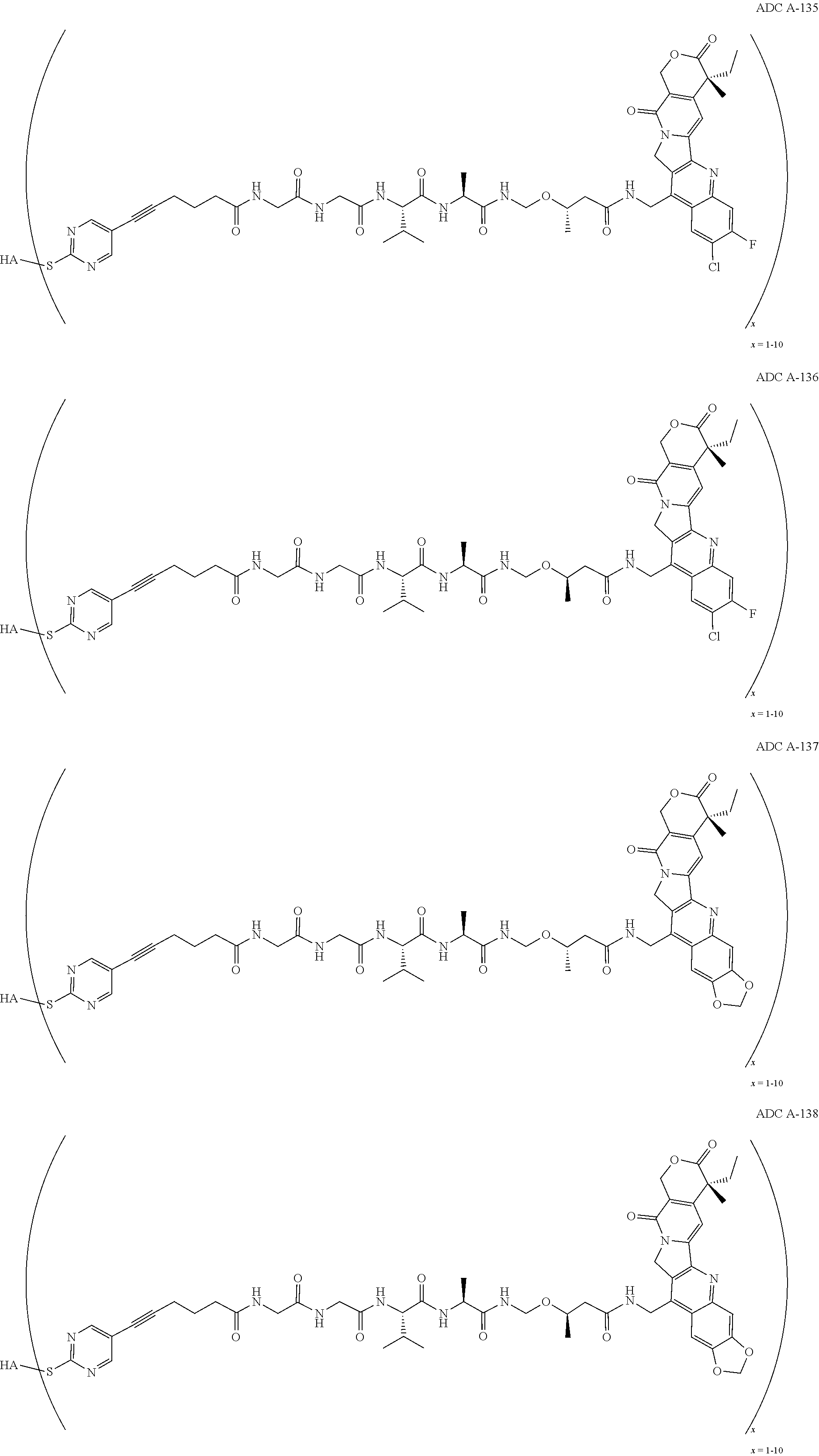 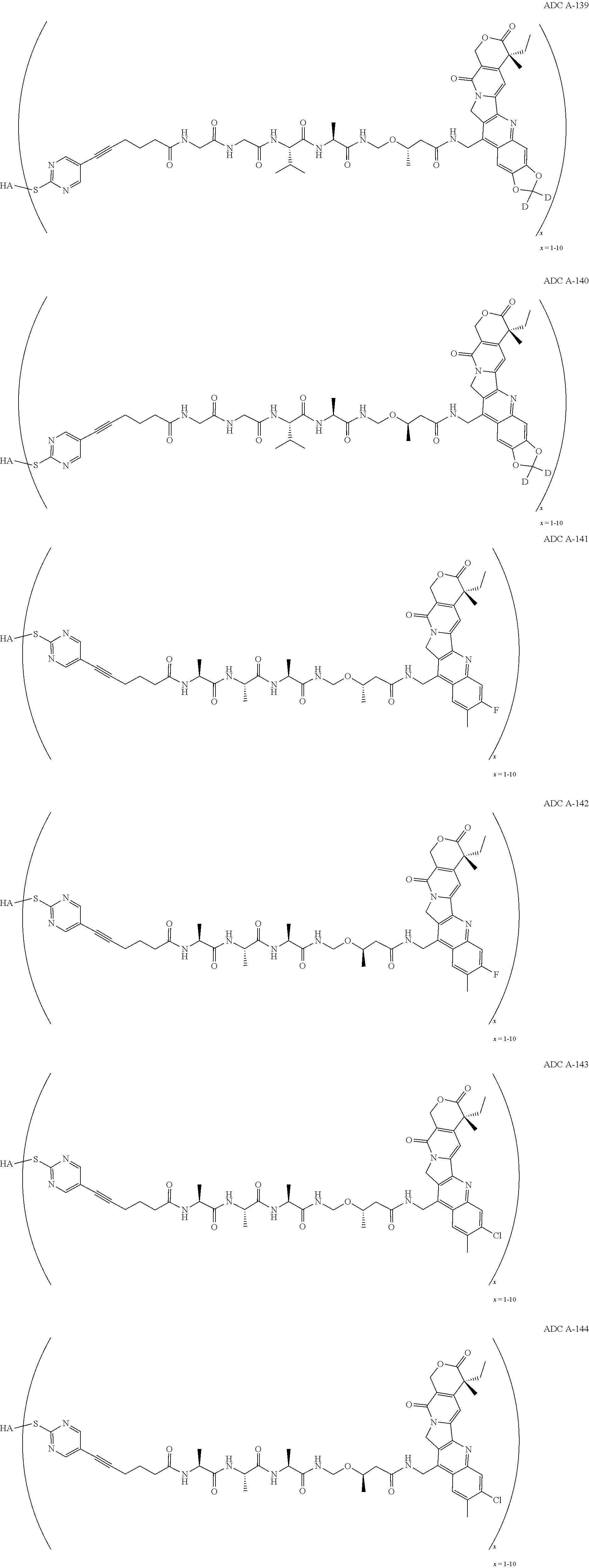 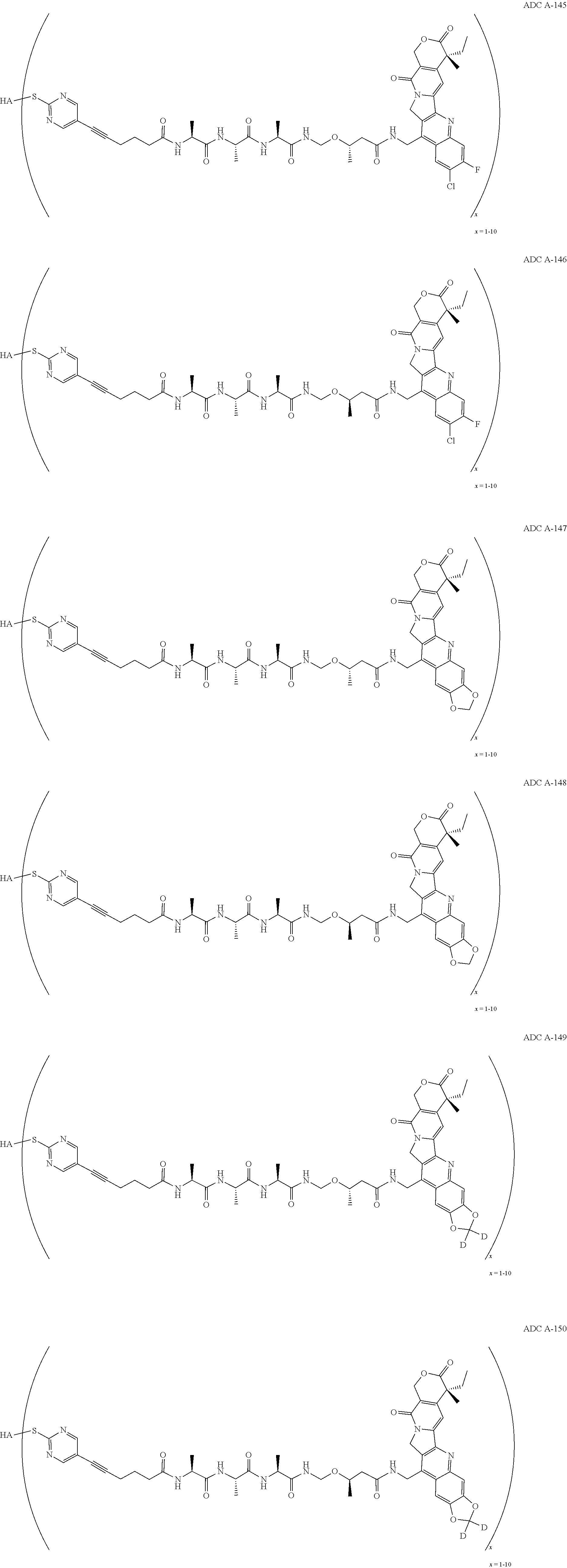 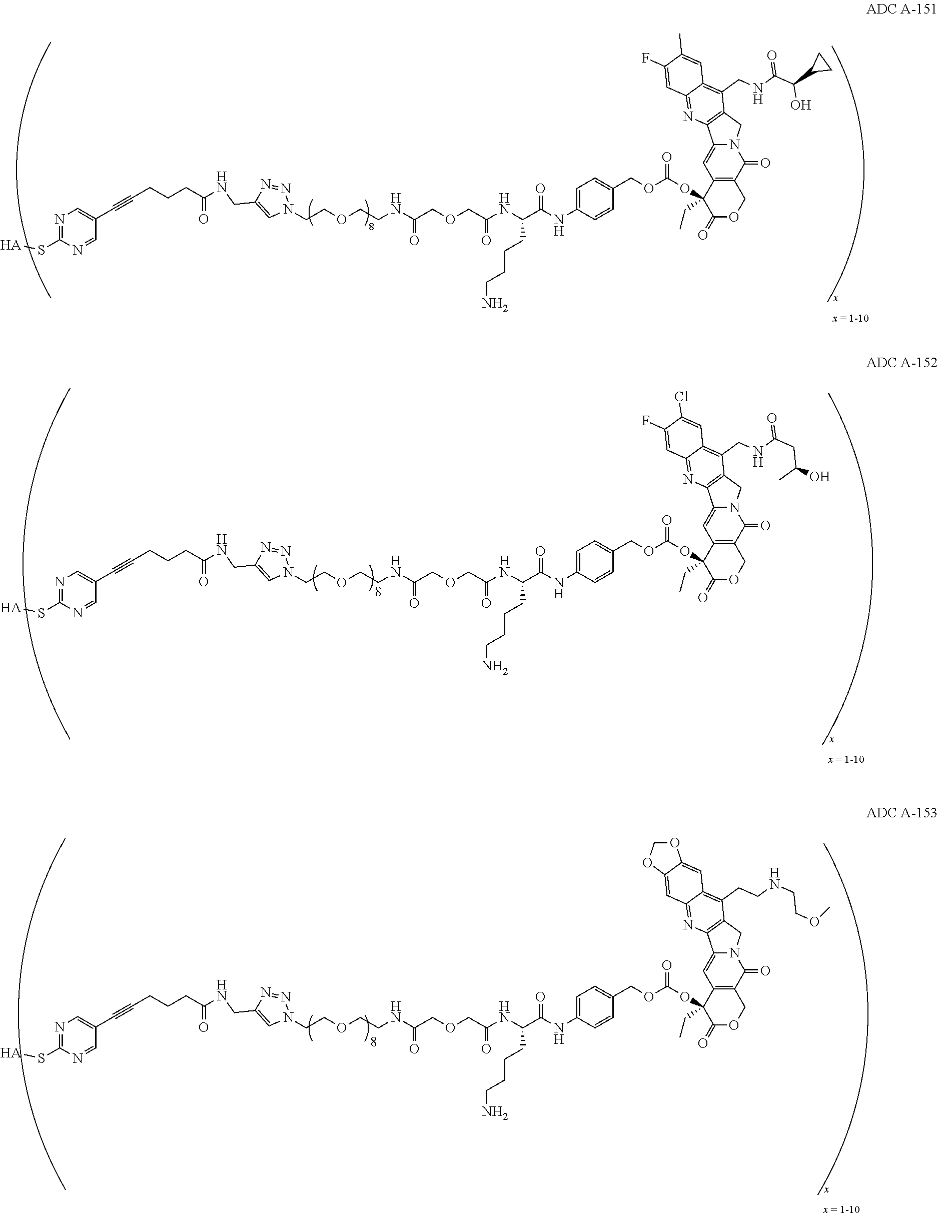 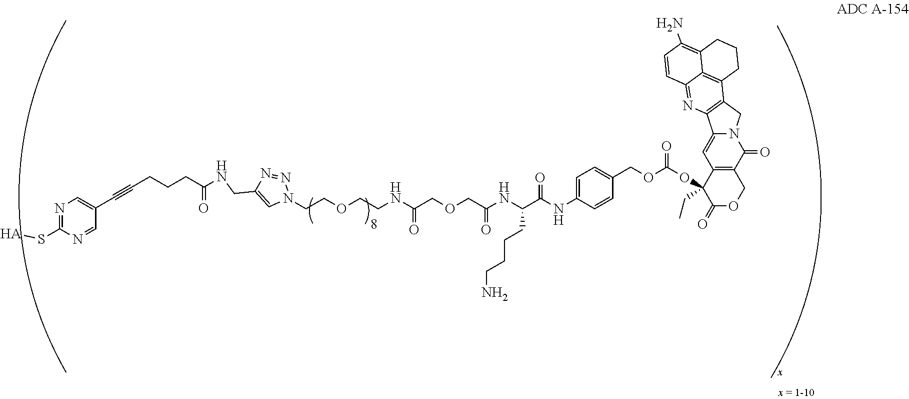 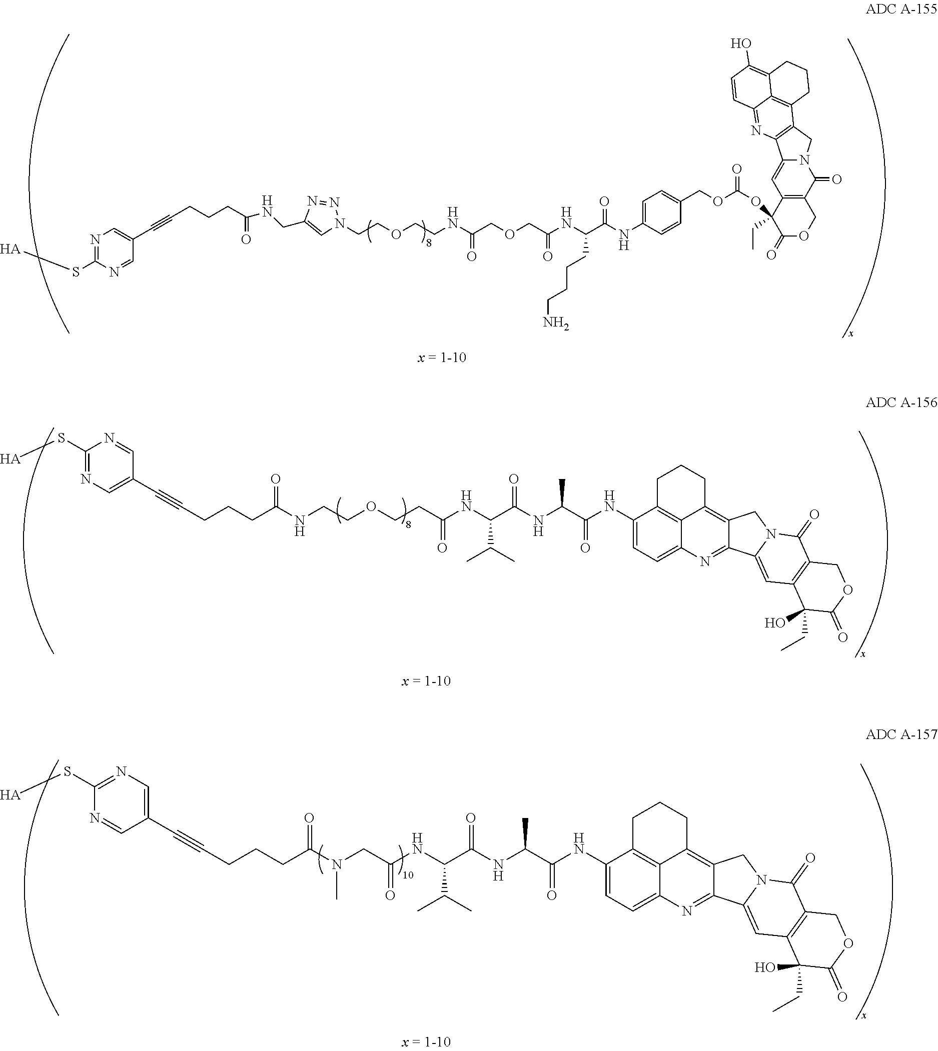 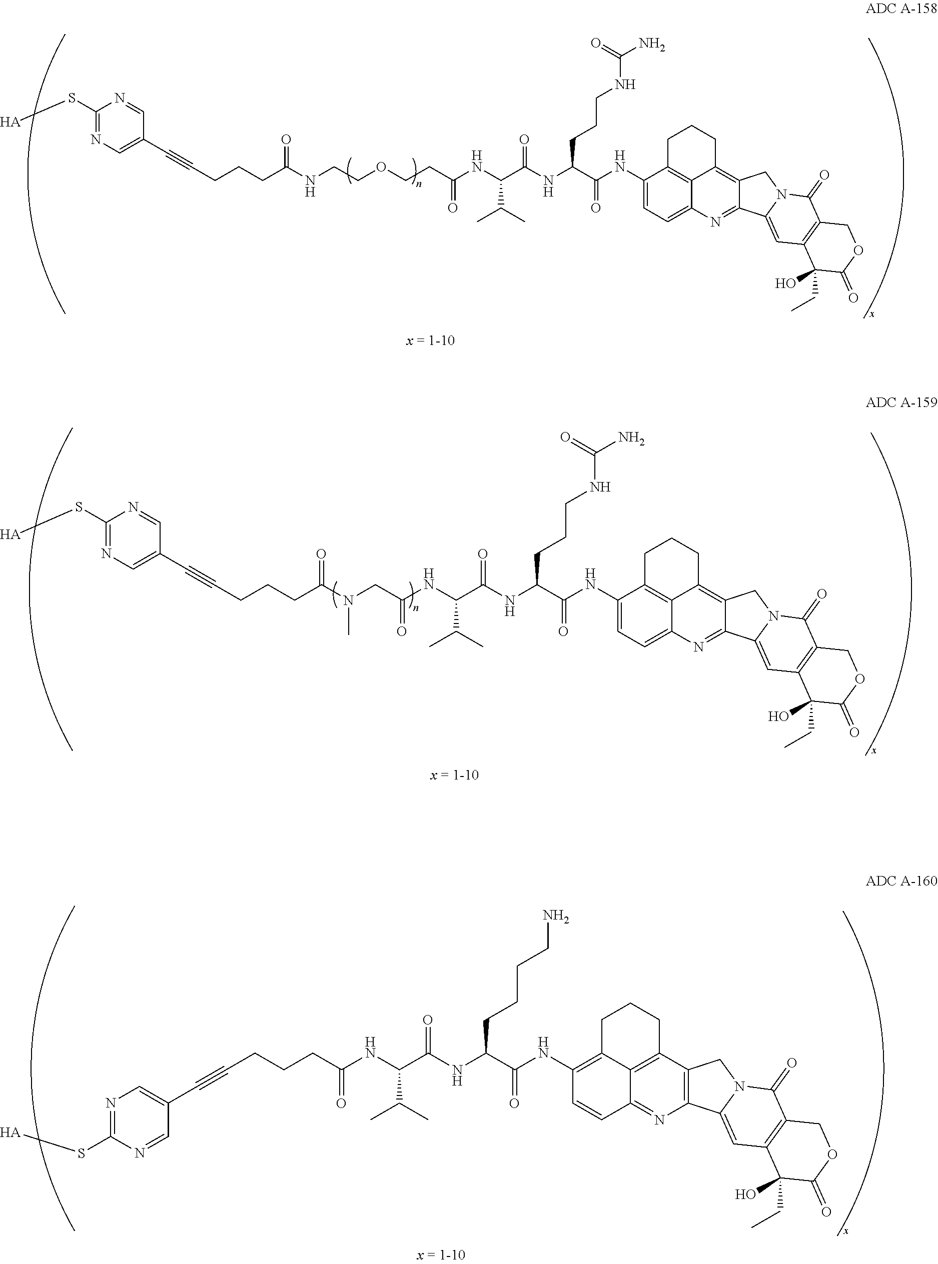 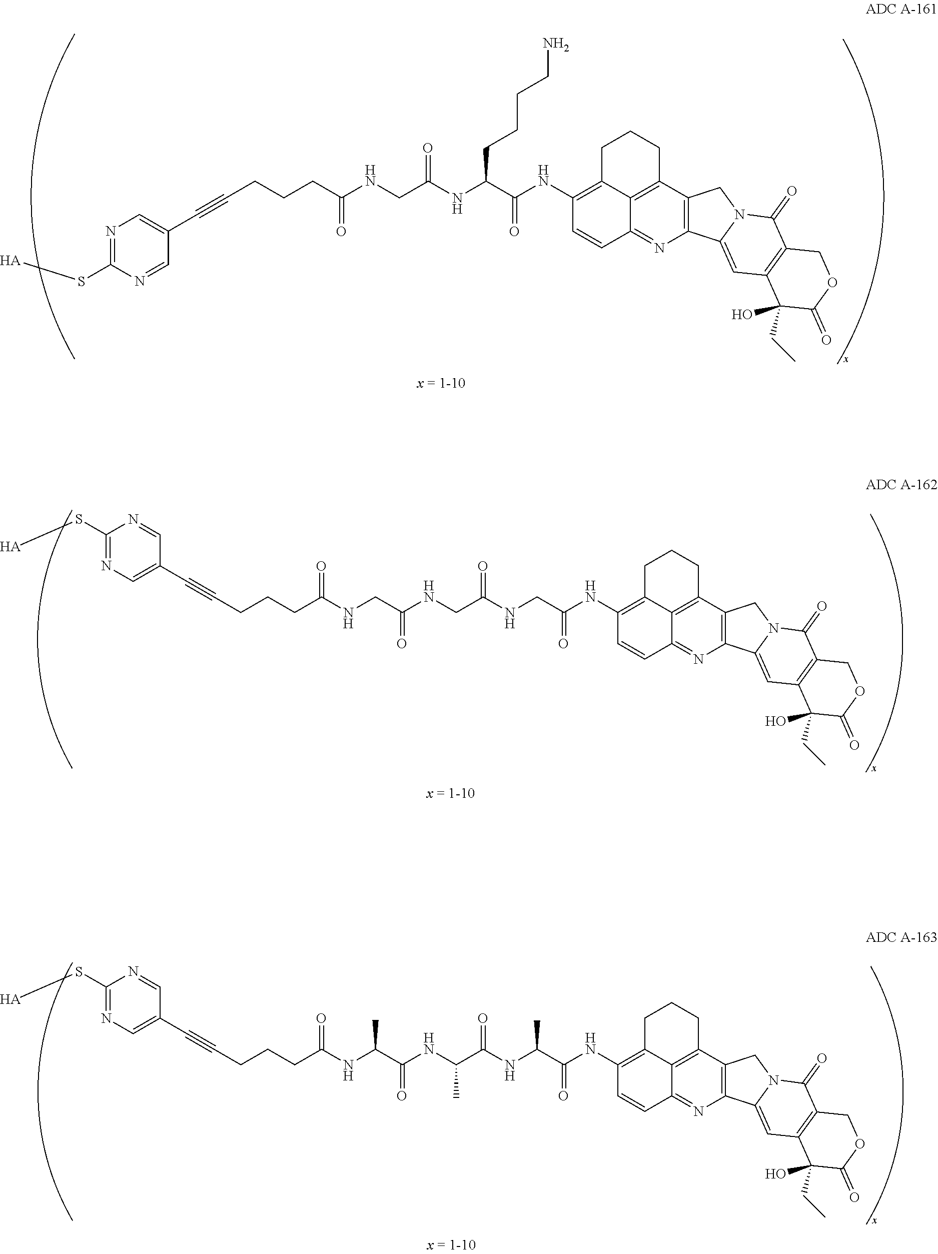 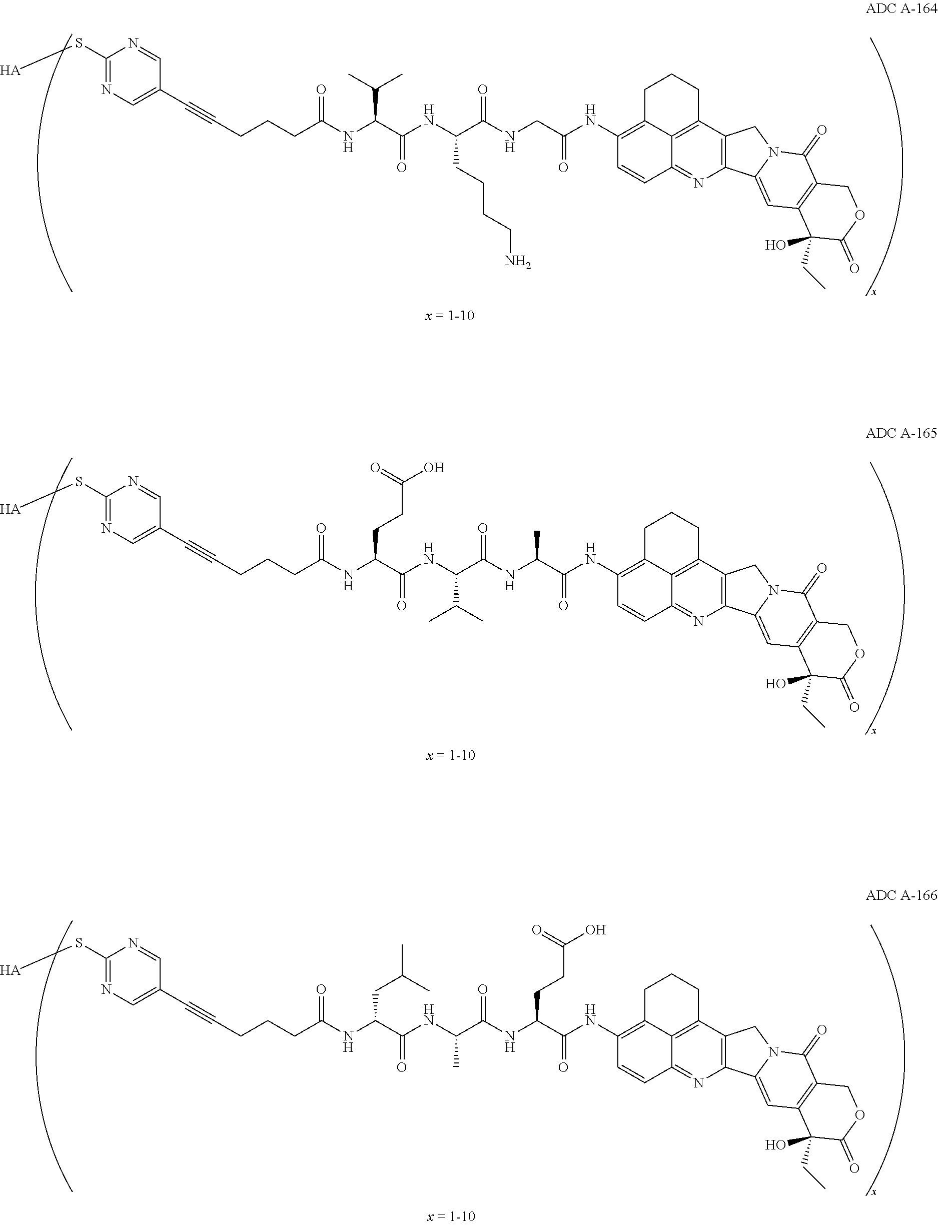 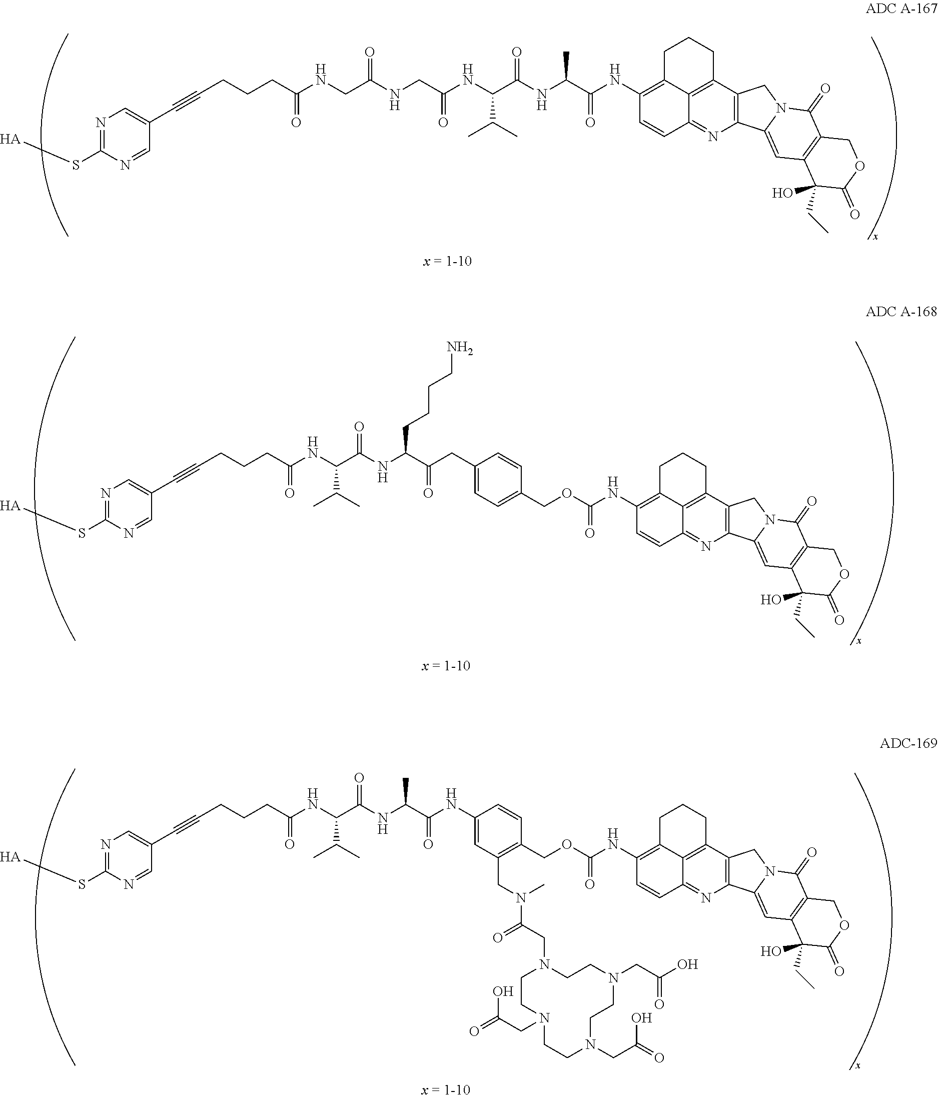 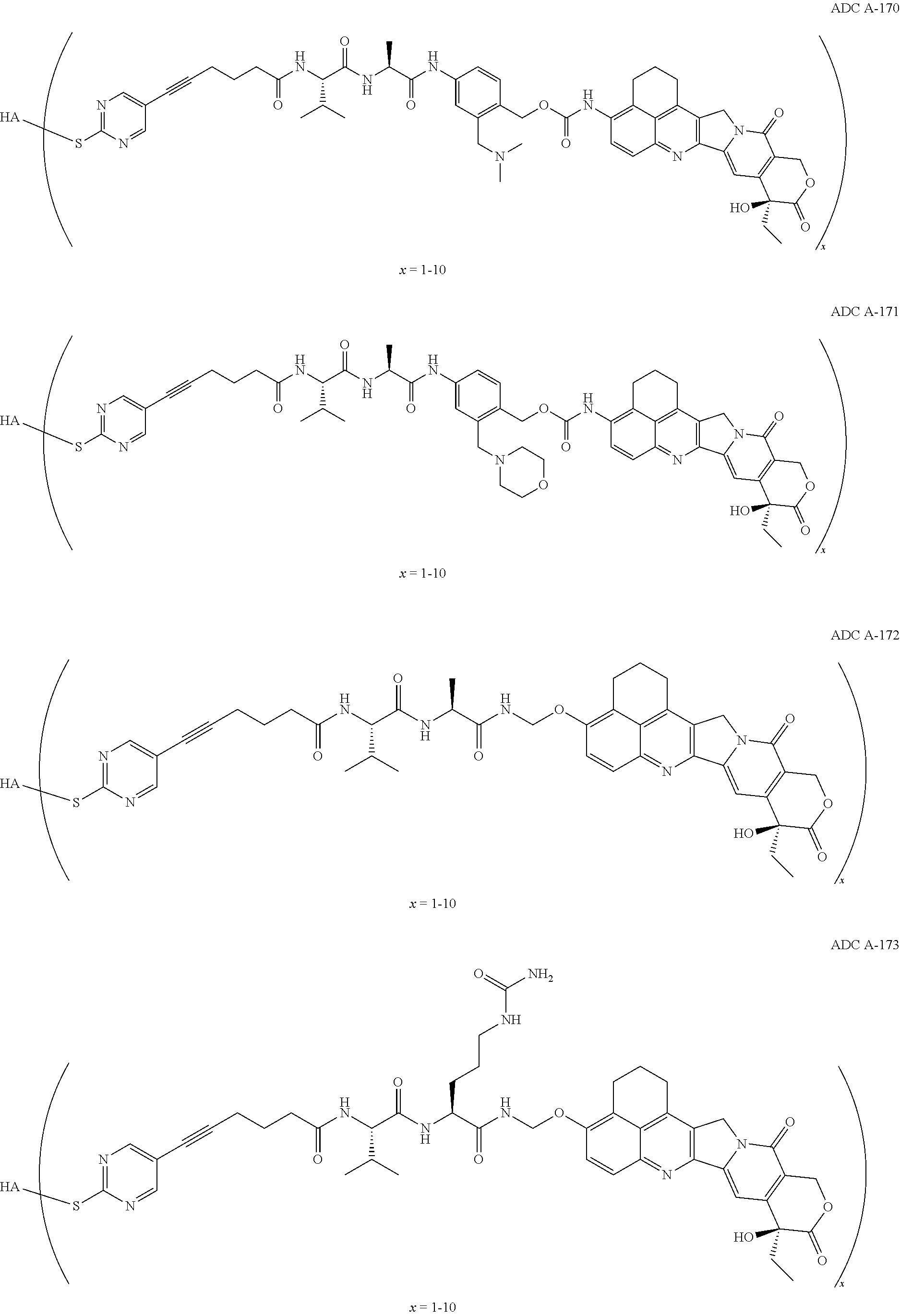 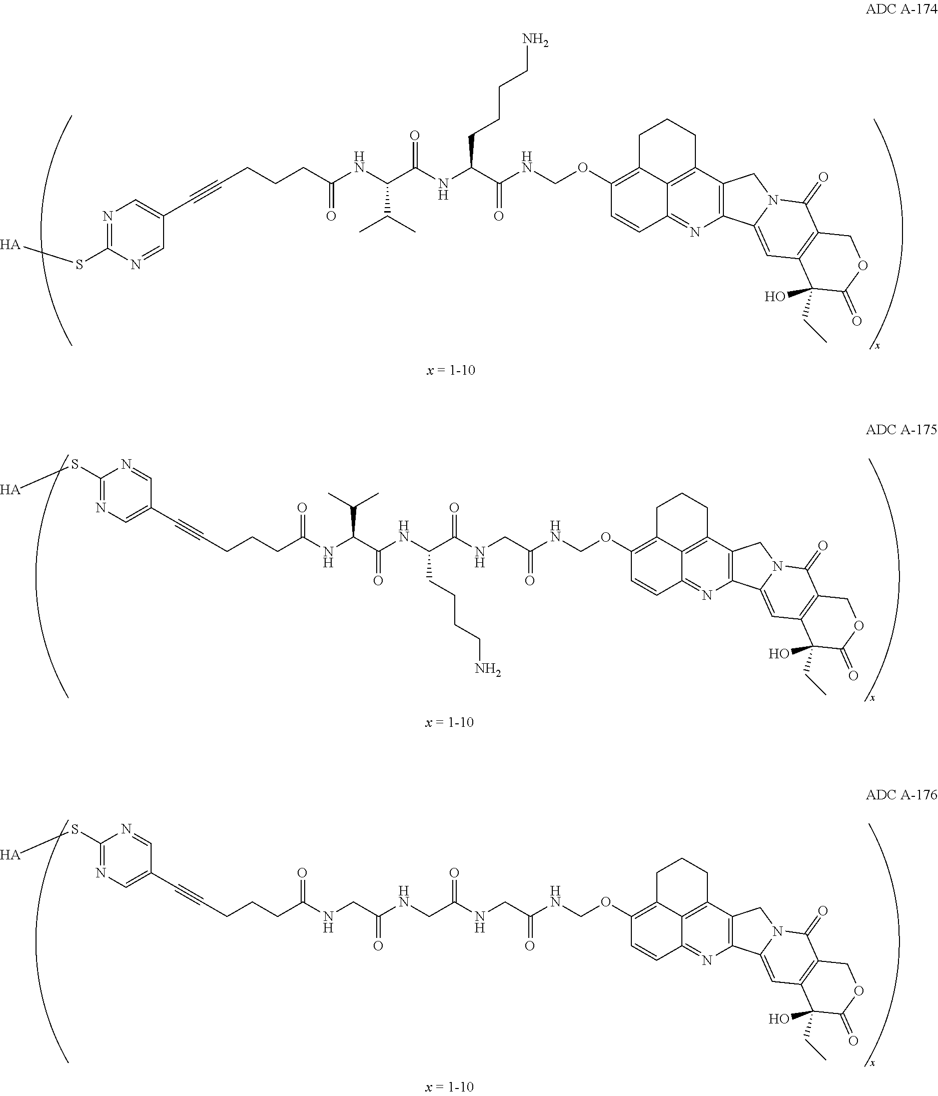 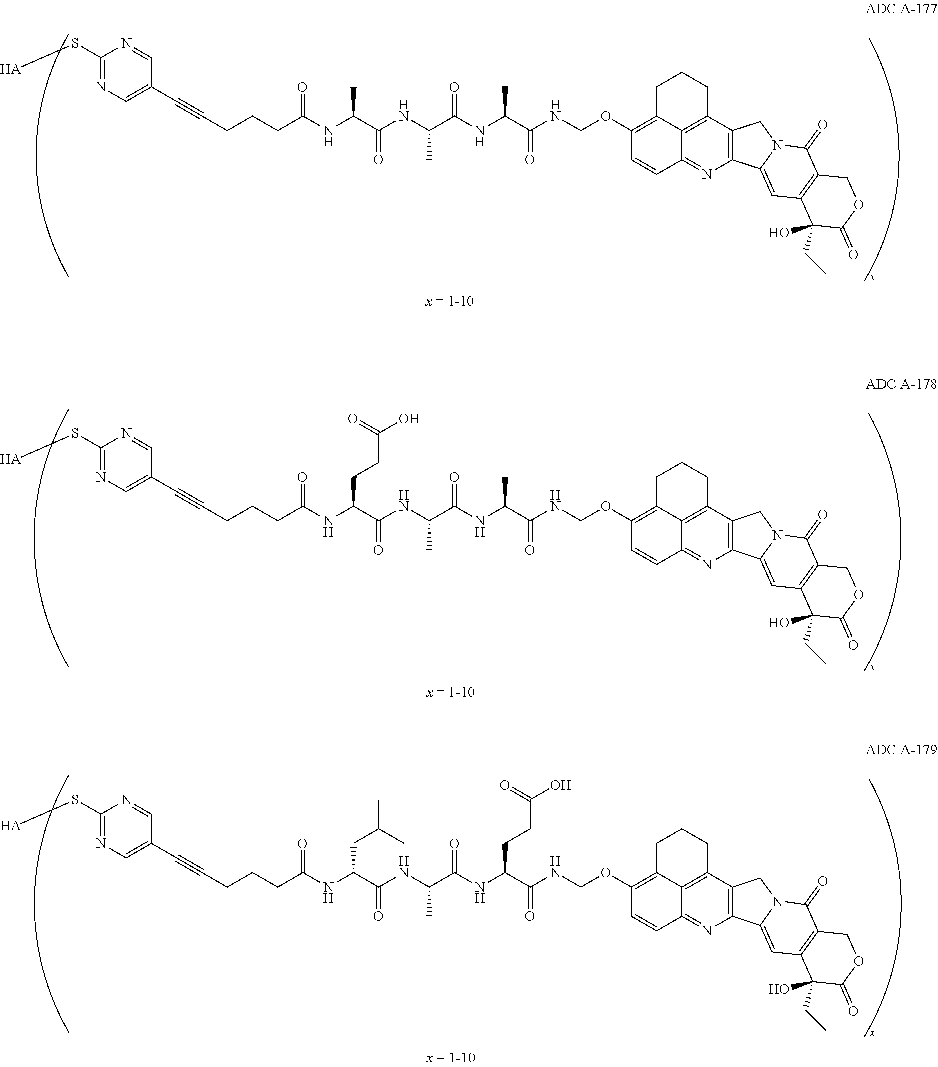 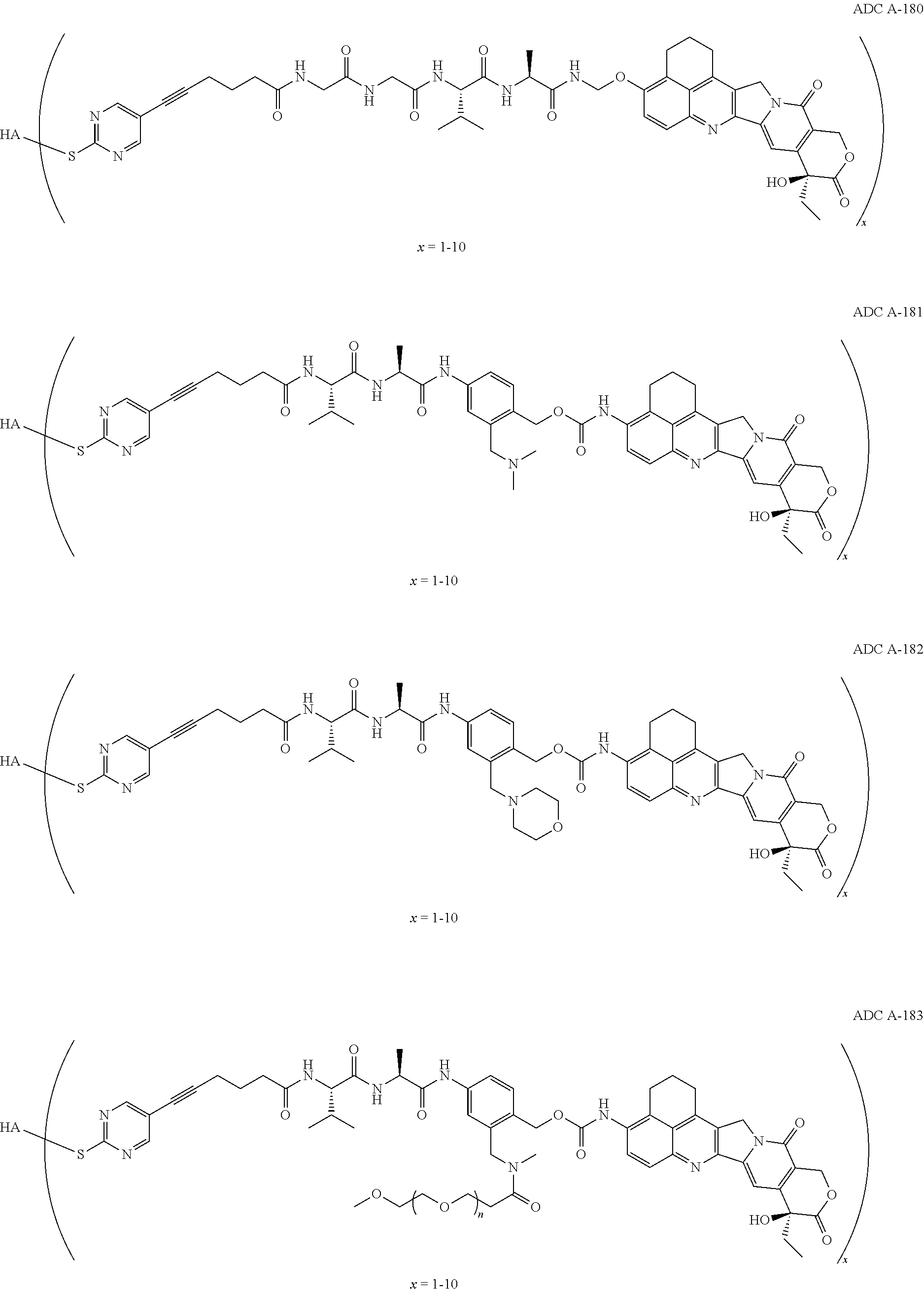 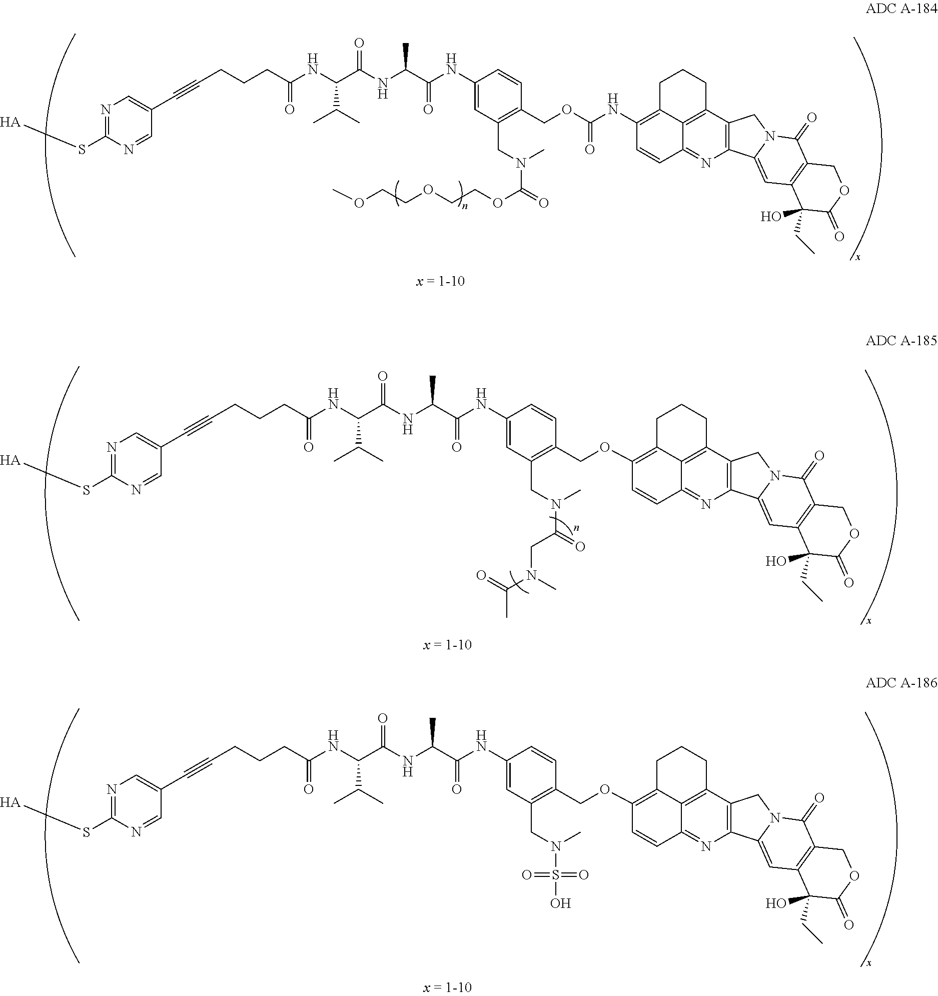 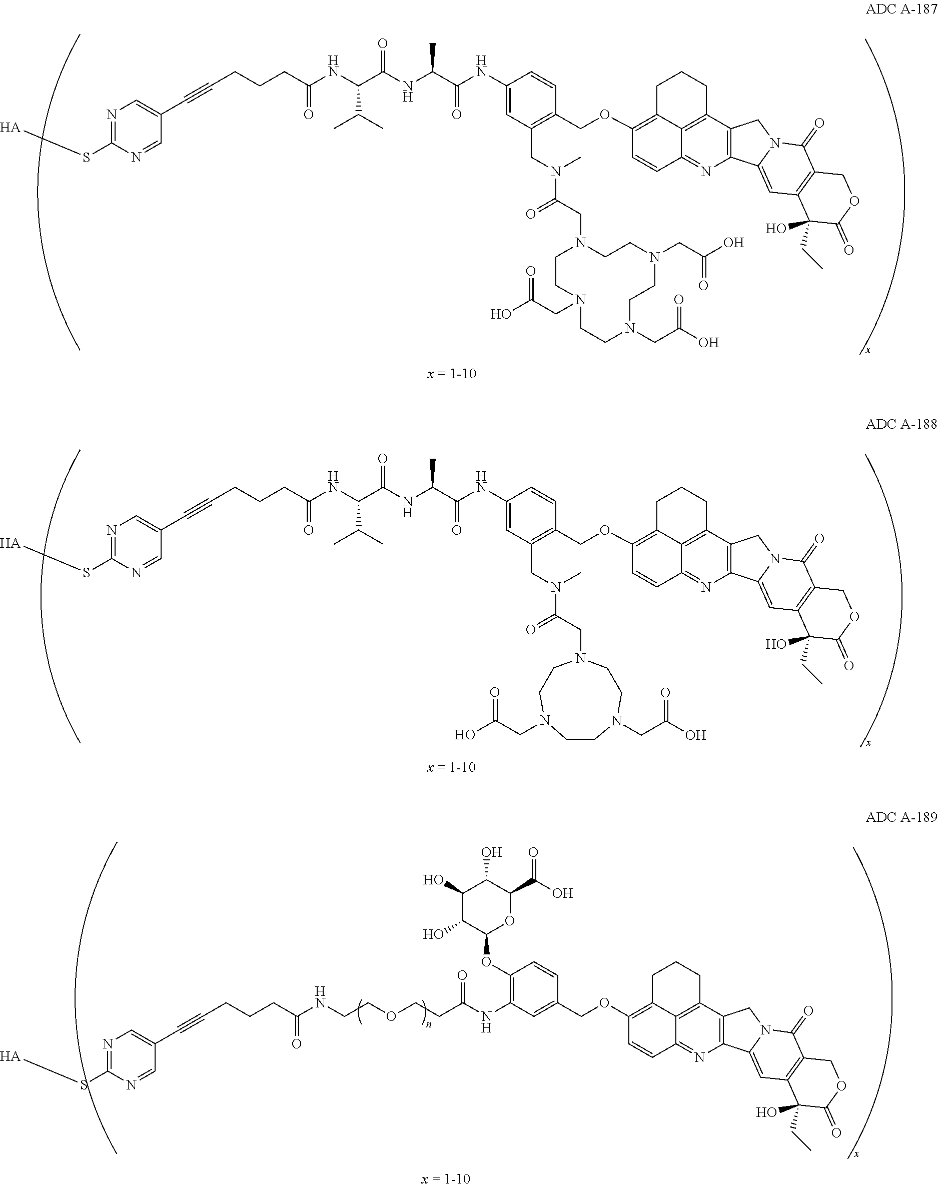 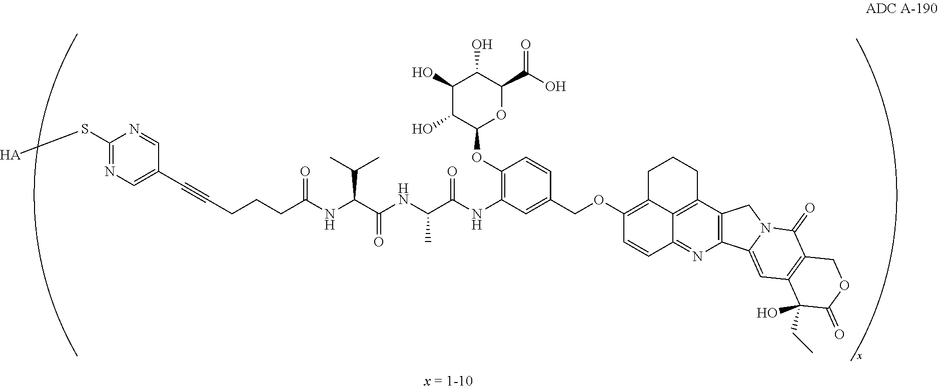 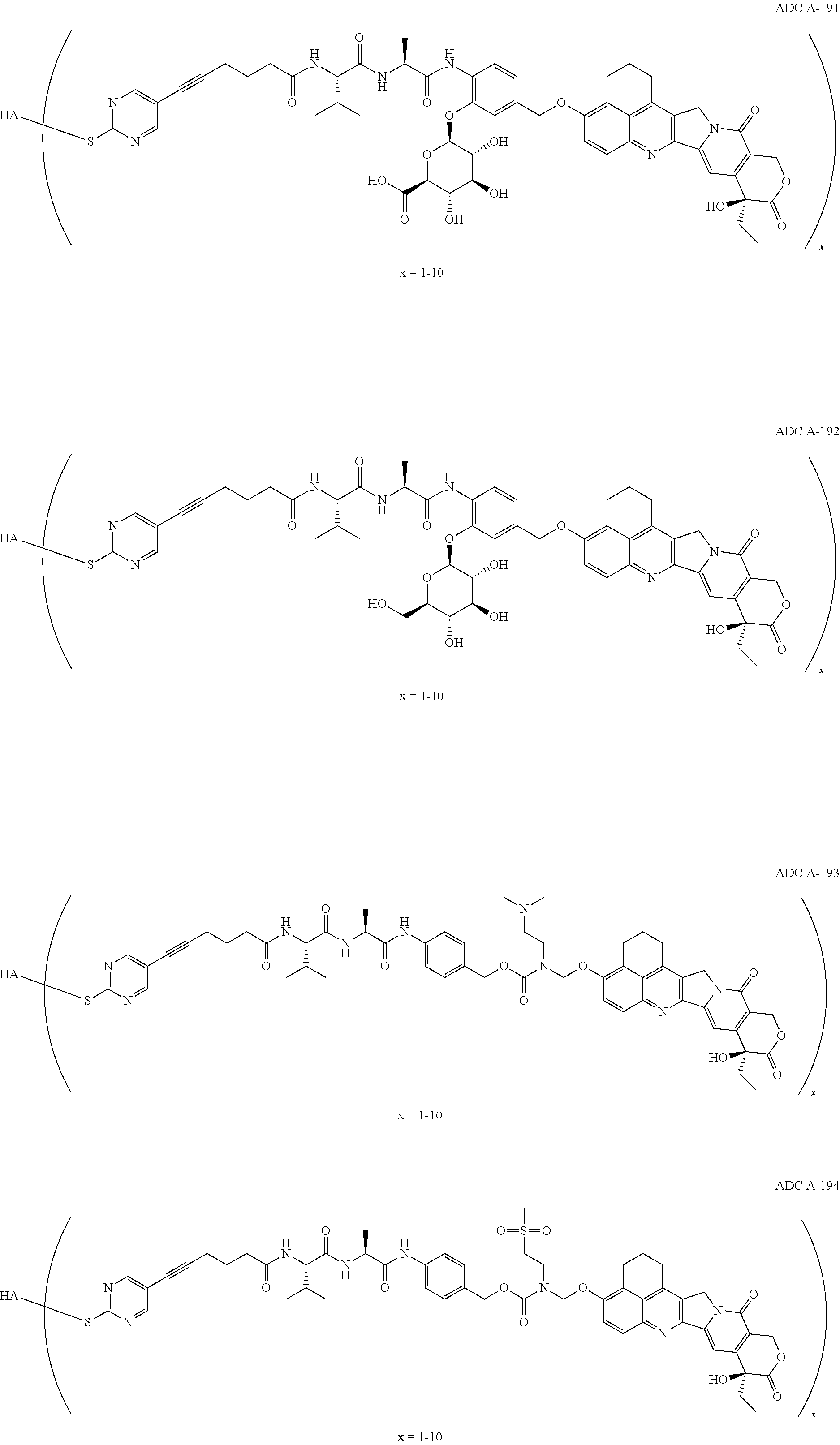 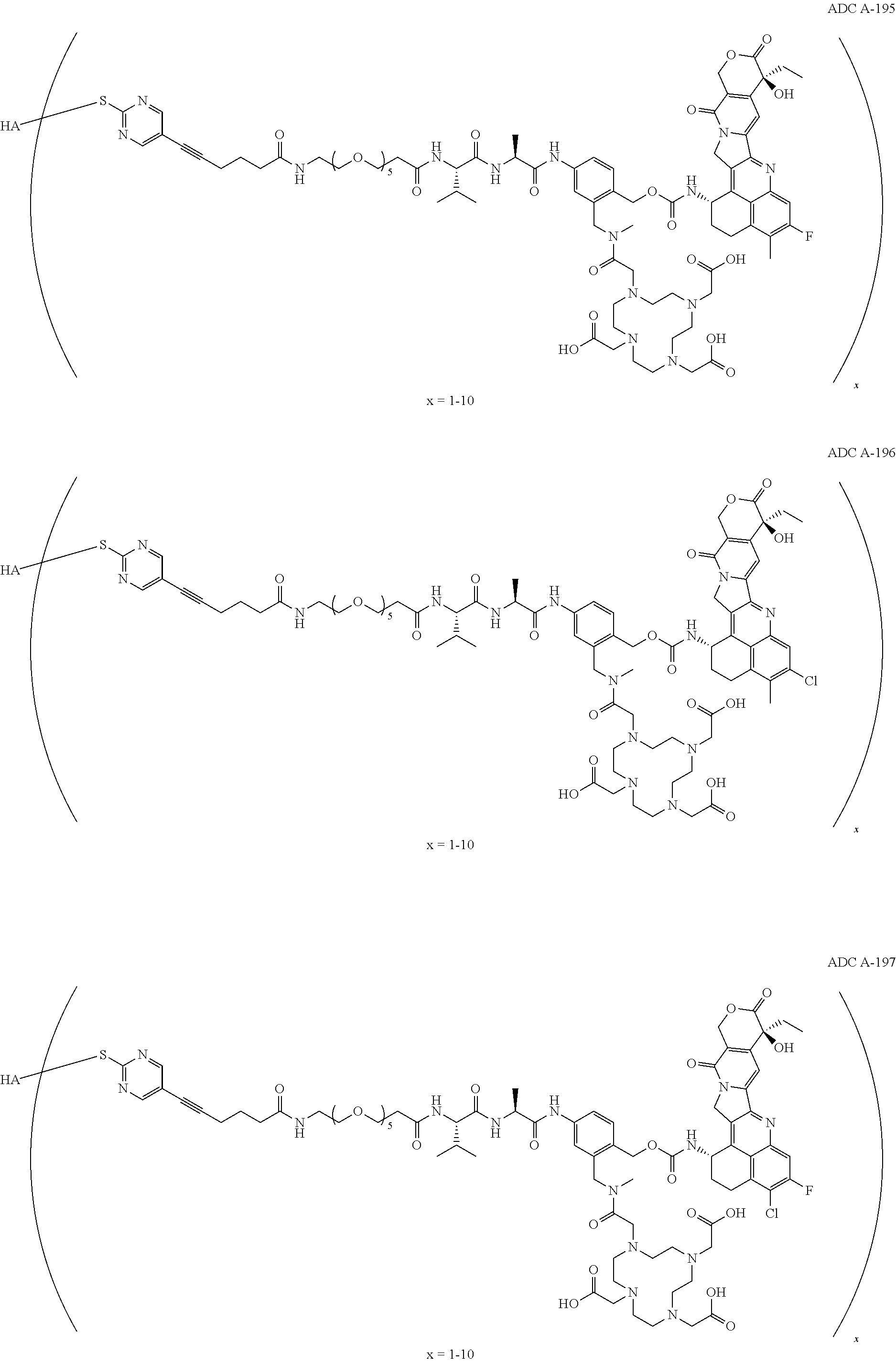 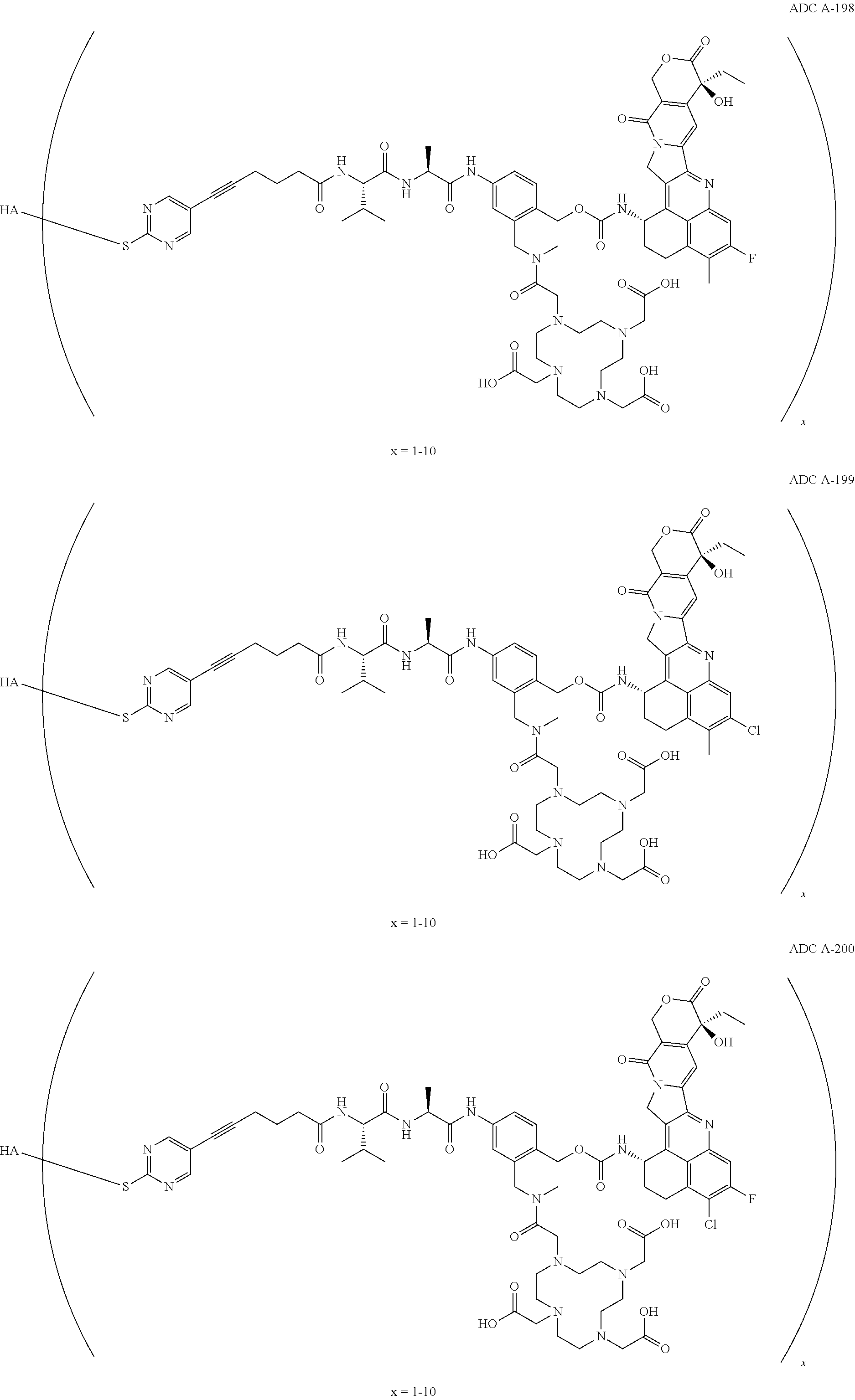 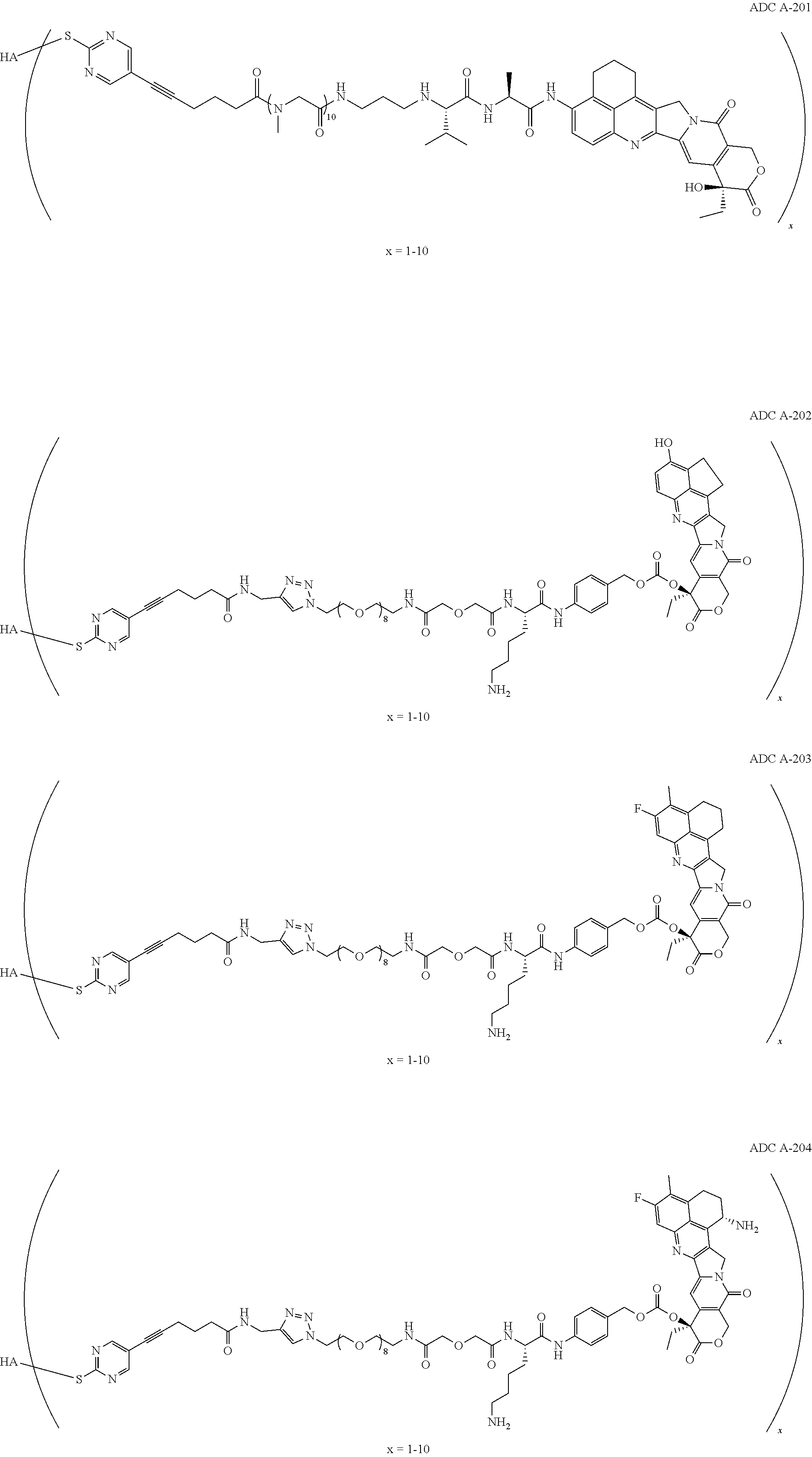 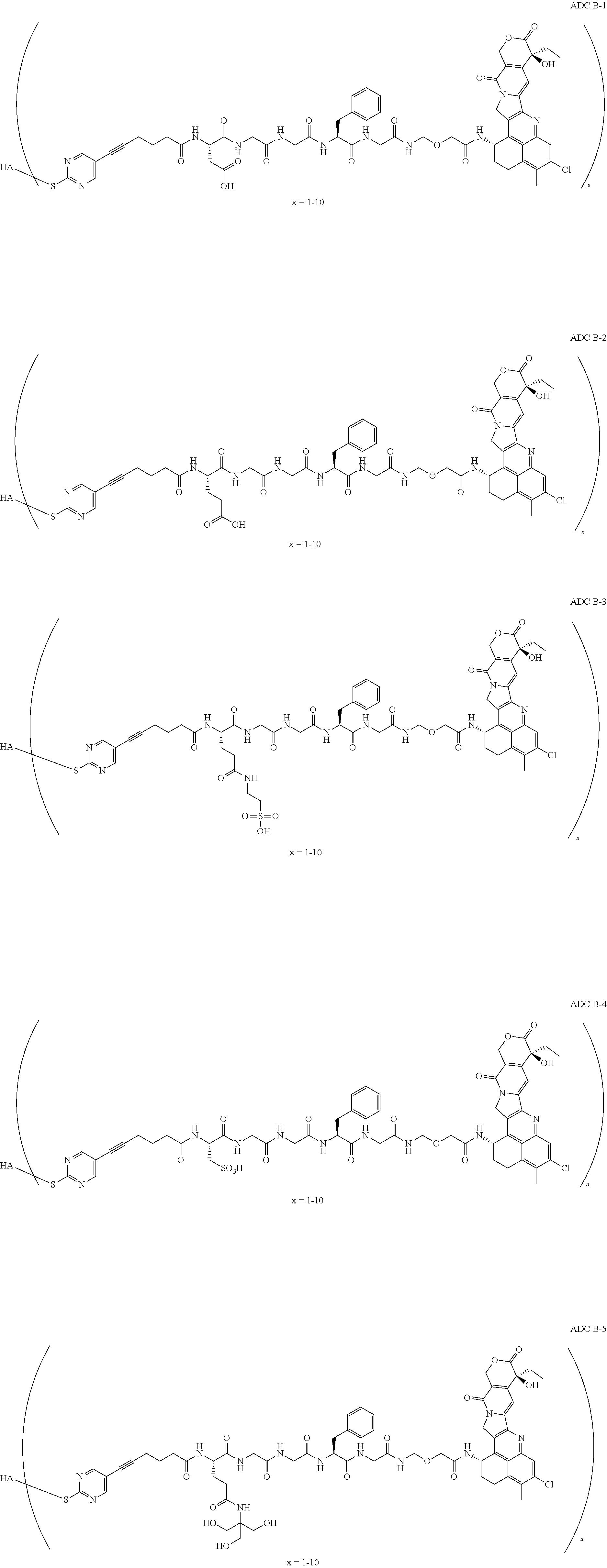 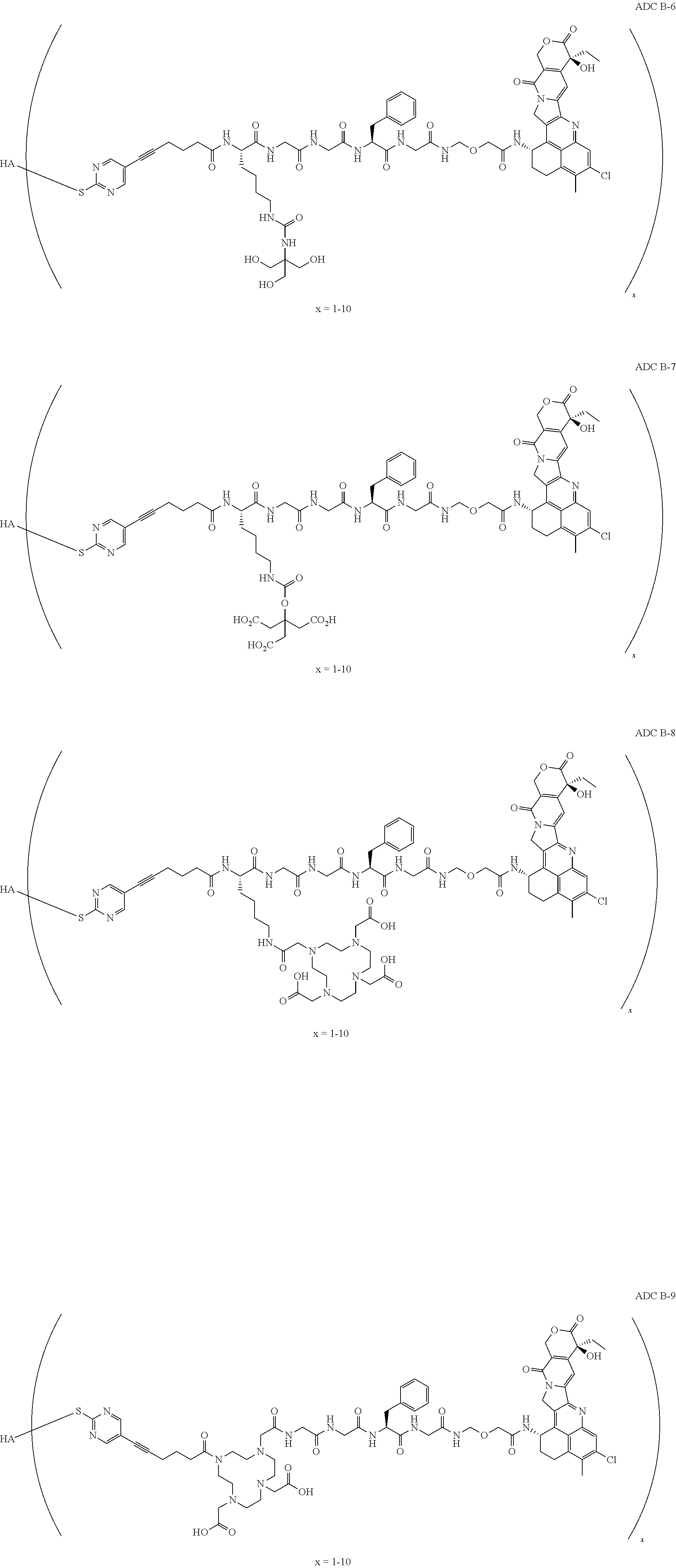 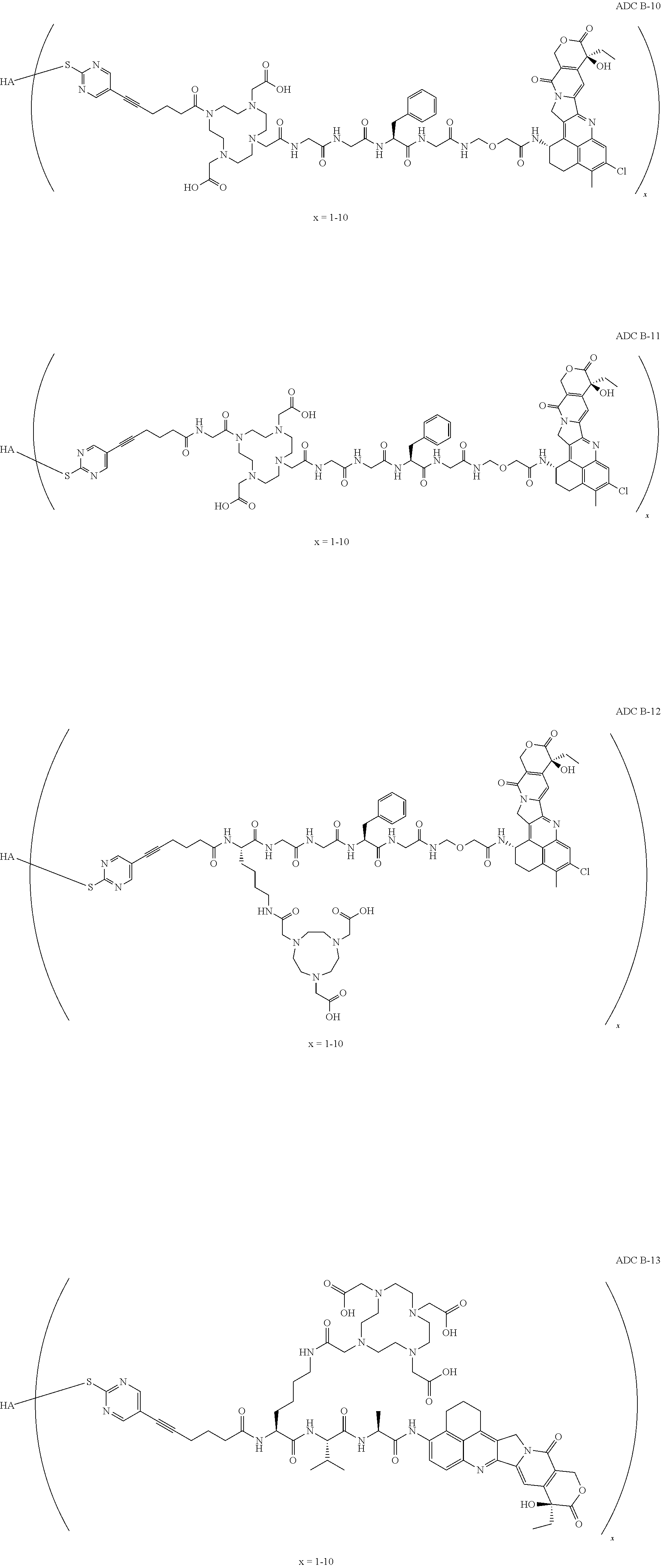 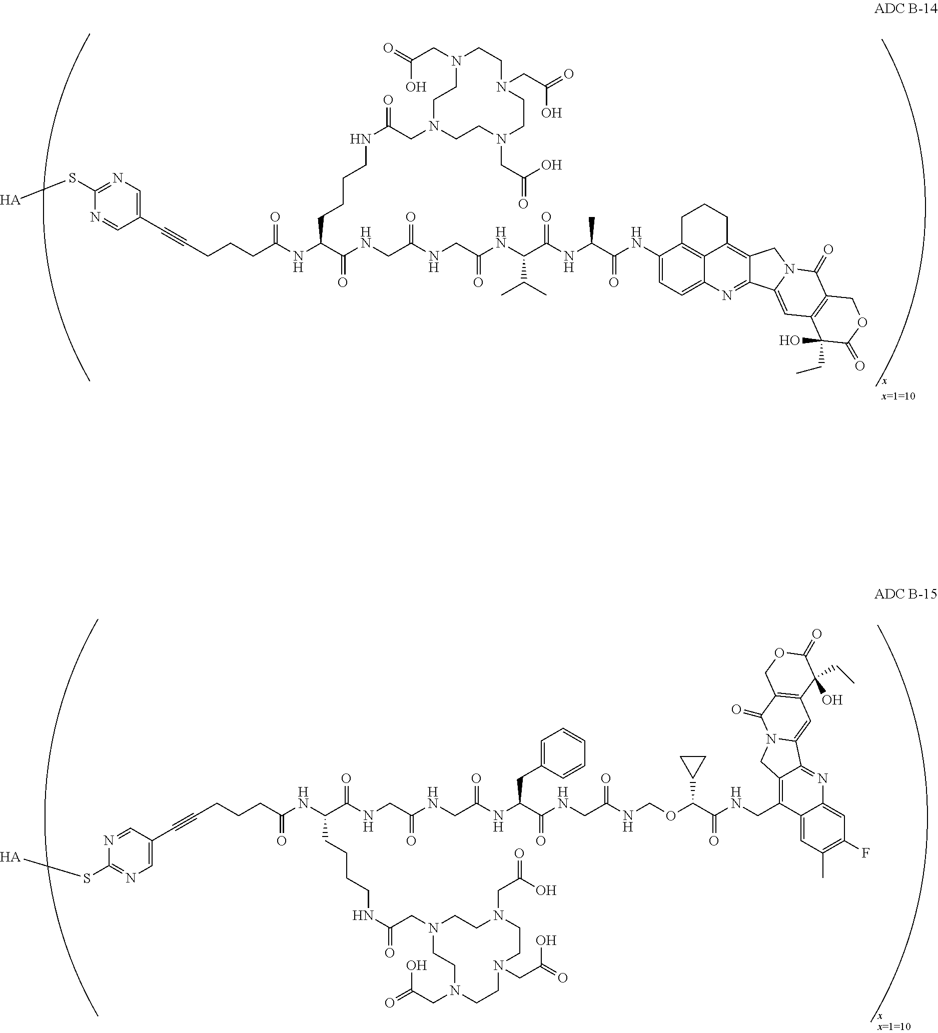 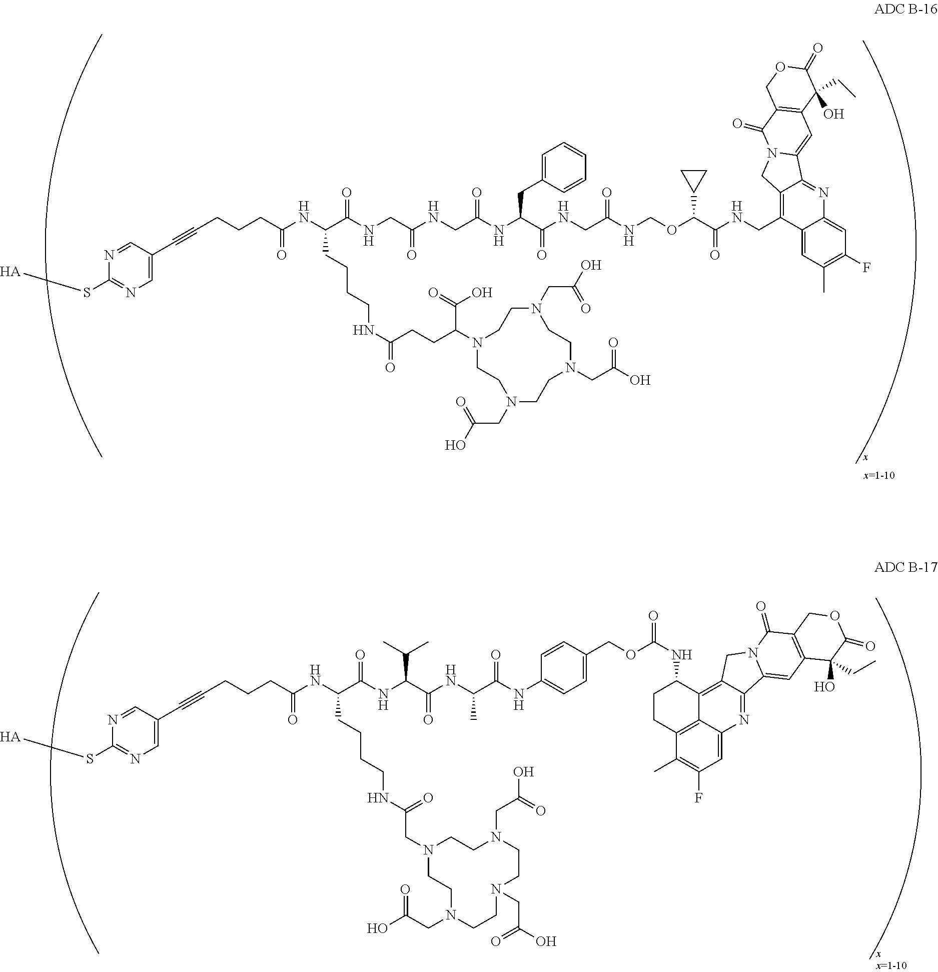 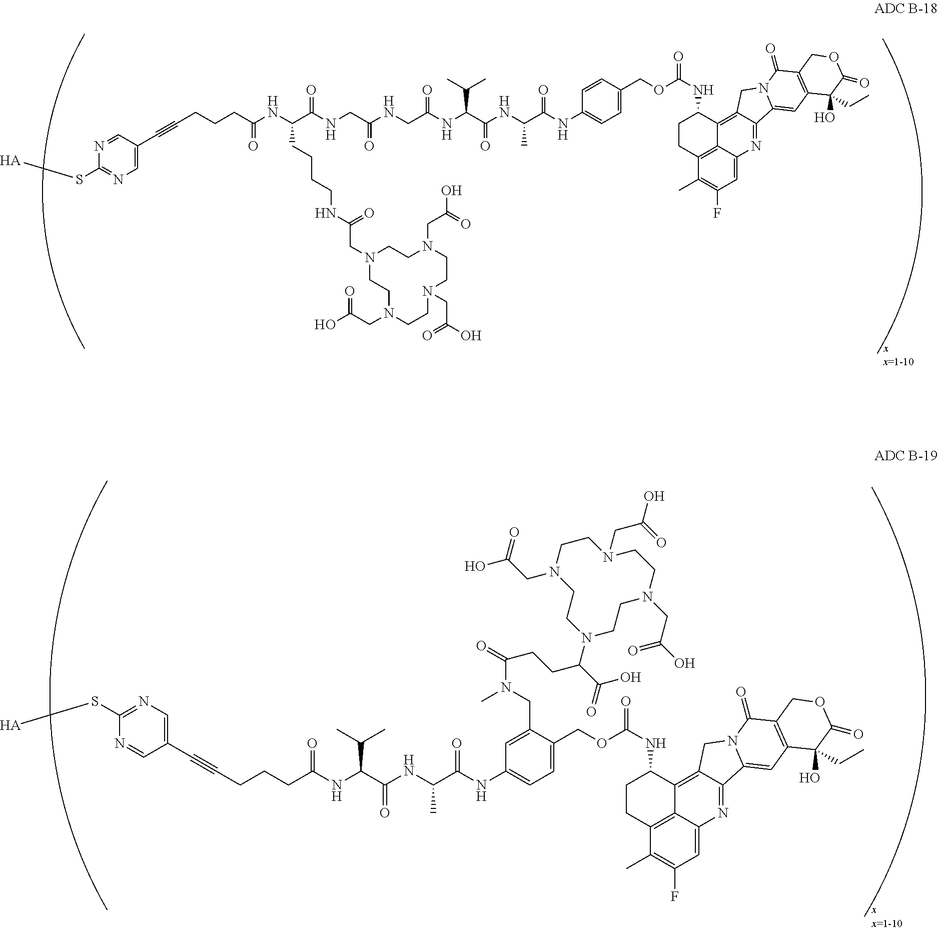 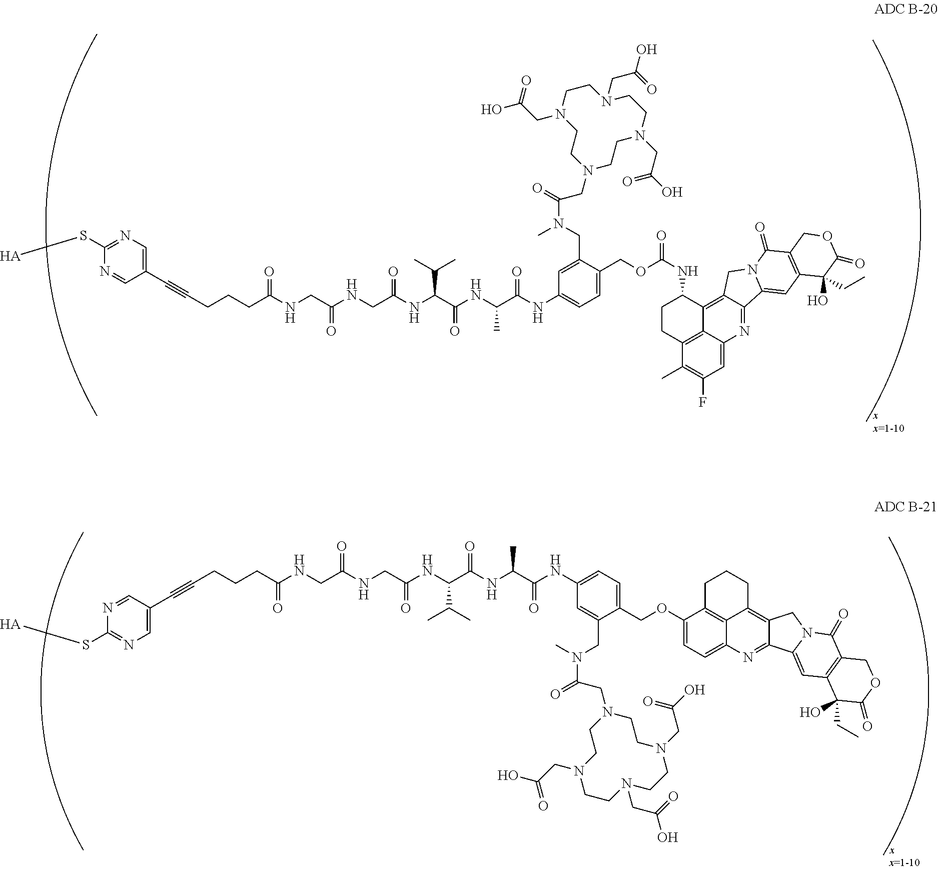 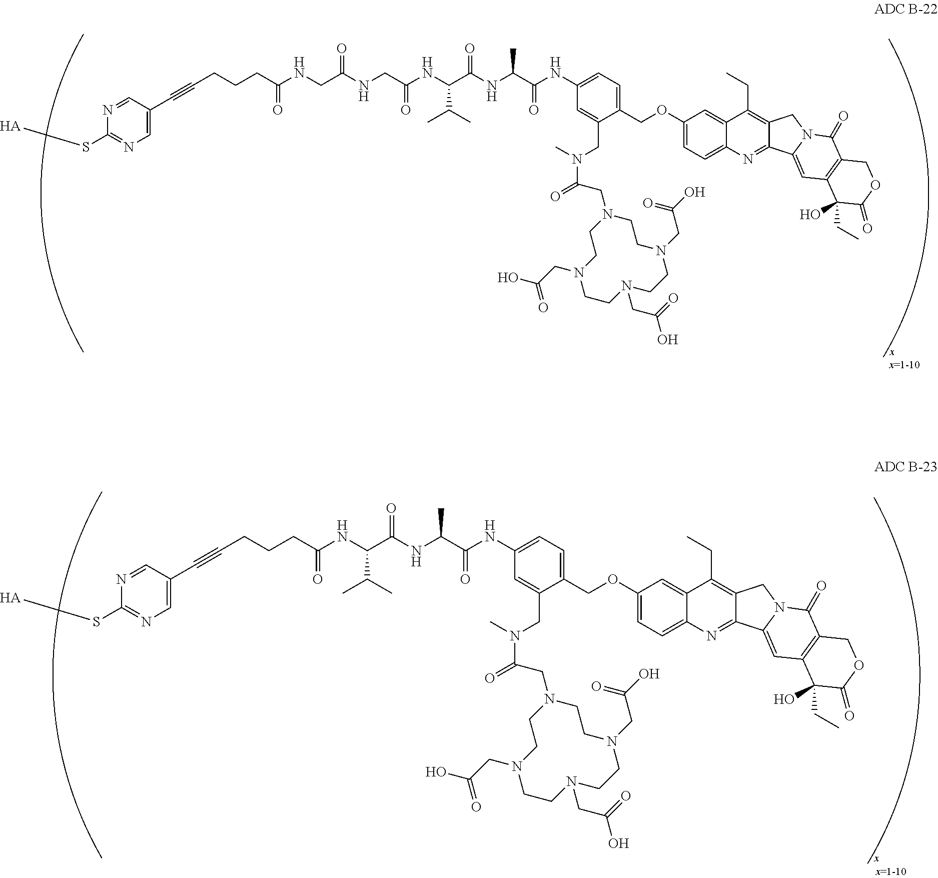 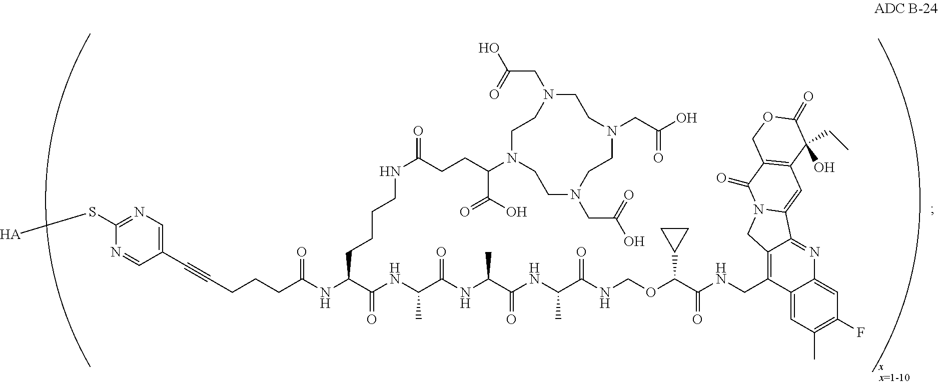
wherein, HA in each antibody-drug conjugate is an antibody or antigen-binding fragment thereof; n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14, 15, 16, 17, 18, 19, or 20;
wherein, 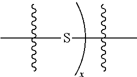 [SMILES: *S*]
represents a specific linkage between a sulfhydryl group in the antibody or antigen-binding fragment thereof and the linker.

11. A composition, the composition comprising one or more conjugates according to claim 8, wherein the DAR value (drug-to-antibody conjugate ratio) of the composition is 1 to 10, for example: 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10, preferably 3 to 8, for example, 3.0 to 3.5, 3.0 to 4.0, 3.0 to 4.5, 3.0 to 5.0, 6.0 to 6.5, 6.0 to 7.0, 6.0 to 7.5, 6.0 to 8.0, 6.0 to 8.5, 6.5 to 7.0, 6.5 to 7.5, 6.5 to 8.0, 6.5 to 8.5, 7.0 to 7.5, 7.0 to 8.0 or 7.5 to 8.0; alternatively, the composition has a DAR of about 6.0 to 9.0, preferably about 6.0 to 8.0, for example, about 6.0, about 6.01, about 6.02, about 6.03, about 6.04, about 6.05, about 6.06, about 6.07, about 6.08, about 6.09, about 6.1, about 6.11, about 6.12, about 6.13, about 6.14, about 6.15, about 6.16, about 6.17, about 6.18, about 6.19, about 6.2, about 6.21, about 6.22, about 6.23, about 6.24, about 6.25, about 6.26, about 6.27, about 6.28, about 6.29, about 6.3, about 6.31, about 6.32, about 6.33, about 6.34, about 6.35, about 6.36, about 6.37, about 6.38, about 6.39, about 6.4, about 6.41, about 6.42, about 6.43, about 6.44, about 6.45, about 6.46, about 6.47, about 6.48, about 6.49, about 6.5, about 6.51, about 6.52, about 6.53, about 6.54, about 6.55, about 6.56, about 6.57, about 6.58, about 6.59, about 6.6, about 6.61, about 6.62, about 6.63, about 6.64, about 6.65, about 6.66, about 6.67, about 6.68, about 6.69, about 6.7, about 6.71, about 6.72, about 6.73, about 6.74, about 6.75, about 6.76, about 6.77, about 6.78, about 6.79, about 6.8, about 6.81, about 6.82, about 6.83, about 6.84, about 6.85, about 6.86, about 6.87, about 6.88, about 6.89, about 6.9, about 6.91, about 6.92, about 6.93, about 6.94, about 6.95, about 6.96, about 6.97, about 6.98, about 6.99, about 7.0, about 7.01, about 7.02, about 7.03, about 7.04, about 7.05, about 7.06, about 7.07, about 7.08, about 7.09, about 7.1, about 7.11, about 7.12, about 7.13, about 7.14, about 7.15, about 7.16, about 7.17, about 7.18, about 7.19, about 7.2, about 7.21, about 7.22, about 7.23, about 7.24, about 7.25, about 7.26, about 7.27, about 7.28, about 7.29, about 7.3, about 7.31, about 7.32, about 7.33, about 7.34, about 7.35, about 7.36, about 7.37, about 7.38, about 7.39, about 7.4, about 7.41, about 7.42, about 7.43, about 7.44, about 7.45, about 7.46, about 7.47, about 7.48, about 7.49, about 7.5, about 7.51, about 7.52, about 7.53, about 7.54, about 7.55, about 7.56, about 7.57, about 7.58, about 7.59, about 7.6, about 7.61, about 7.62, about 7.63, about 7.64, about 7.65, about 7.66, about 7.67, about 7.68, about 7.69, about 7.7, about 7.71, about 7.72, about 7.73, about 7.74, about 7.75, about 7.76, about 7.77, about 7.78, about 7.79, about 7.8, about 7.81, about 7.82, about 7.83, about 7.84, about 7.85, about 7.86, about 7.87, about 7.88, about 7.89, about 7.9, about 7.91, about 7.92, about 7.93, about 7.94, about 7.95, about 7.96, about 7.97, about 7.98, about 7.99, about 8.0, about 8.01, about 8.02, about 8.03, about 8.04, about 8.05, about 8.06, about 8.07, about 8.08, about 8.09, about 8.1, about 8.11, about 8.12, about 8.13, about 8.14, about 8.15, about 8.16, about 8.17, about 8.18, about 8.19, about 8.2, about 8.21, about 8.22, about 8.23, about 8.24, about 8.25, about 8.26, about 8.27, about 8.28, about 8.29, about 8.3, about 8.31, about 8.32, about 8.33, about 8.34, about 8.35, about 8.36, about 8.37, about 8.38, about 8.39, about 8.4, about 8.41, about 8.42, about 8.43, about 8.44, about 8.45, about 8.46, about 8.47, about 8.48, about 8.49, about 8.5, about 8.51, about 8.52, about 8.53, about 8.54, about 8.55, about 8.56, about 8.57, about 8.58, about 8.59, about 8.6, about 8.61, about 8.62, about 8.63, about 8.64, about 8.65, about 8.66, about 8.67, about 8.68, about 8.69, about 8.7, about 8.71, about 8.72, about 8.73, about 8.74, about 8.75, about 8.76, about 8.77, about 8.78, about 8.79, about 8.8, about 8.81, about 8.82, about 8.83, about 8.84, about 8.85, about 8.86, about 8.87, about 8.88, about 8.89, about 8.9, about 8.91, about 8.92, about 8.93, about 8.94, about 8.95, about 8.96, about 8.97, about 8.98, about 8.99, about 9.0.

12. A pharmaceutical composition, which comprises the compound or pharmaceutically acceptable salt thereof according to claim 1, and one or more pharmaceutical excipients.

13-16. (canceled)

17. A method for treating cancer, comprising administering to an individual in need thereof a therapeutically effective amount of the compound or pharmaceutically acceptable salt thereof according to claim 1; preferably, wherein the cancer is selected from the group consisting of solid tumors or hematological malignancies, for example, gastric cancer, breast cancer, lung cancer (for example, non-small cell lung cancer, specifically lung adenocarcinoma) or urothelial carcinoma.

18. (canceled)

19. A pharmaceutical composition, which comprises the conjugate according to claim 8, and one or more pharmaceutical excipients.

20. A pharmaceutical composition, which comprises the composition according to claim 11, and one or more pharmaceutical excipients.

21. A method for treating cancer, comprising administering to an individual in need thereof a therapeutically effective amount of the conjugate according to claim 8;
preferably, wherein the cancer is selected from the group consisting of solid tumors or hematological malignancies, for example, gastric cancer, breast cancer, lung cancer (for example, non-small cell lung cancer, specifically lung adenocarcinoma) or urothelial carcinoma.

22. A method for treating cancer, comprising administering to an individual in need thereof a therapeutically effective amount of the composition according to claim 11;
preferably, wherein the cancer is selected from the group consisting of solid tumors or hematological malignancies, for example, gastric cancer, breast cancer, lung cancer (for example, non-small cell lung cancer, specifically lung adenocarcinoma) or urothelial carcinoma.

23. A method for treating cancer, comprising administering to an individual in need thereof a therapeutically effective amount of the pharmaceutical composition according to claim 12;
preferably, wherein the cancer is selected from the group consisting of solid tumors or hematological malignancies, for example, gastric cancer, breast cancer, lung cancer (for example, non-small cell lung cancer, specifically lung adenocarcinoma) or urothelial carcinoma.

24. A method for treating cancer, comprising administering to an individual in need thereof a therapeutically effective amount of the pharmaceutical composition according to claim 19;
preferably, wherein the cancer is selected from the group consisting of solid tumors or hematological malignancies, for example, gastric cancer, breast cancer, lung cancer (for example, non-small cell lung cancer, specifically lung adenocarcinoma) or urothelial carcinoma.

25. A method for treating cancer, comprising administering to an individual in need thereof a therapeutically effective amount of the pharmaceutical composition according to claim 20;
preferably, wherein the cancer is selected from the group consisting of solid tumors or hematological malignancies, for example, gastric cancer, breast cancer, lung cancer (for example, non-small cell lung cancer, specifically lung adenocarcinoma) or urothelial carcinoma.
